(19) United States
(12) Patent Application Publication
(10) Pub. No.: US 2025/0136606 A1
(43) Pub. Date: May 1, 2025

(54) MODULATORS OF TRPML, THEIR COMPOSITIONS AND METHODS OF USE

(71) Applicant: Caraway Therapeutics, Inc., Cambridge, MA (US)

(72) Inventors: Rajesh R. IYENGAR, West Newton, MA (US); Thomas Wai-Ho Lee, Lexington, MA (US); Casey Cameron McComas, Phoenixville, PA (US); Darby R. Schmidt, Arlington, MA (US); John J. Graziotto, Somerville, MA (US)

(73) Assignee: Caraway Therapeutics, Inc., Cambridge, MA (US)

(21) Appl. No.: 18/695,871

(22) PCT Filed: Sep. 29, 2022
(86) PCT No.: PCT/US2022/045210

§ 371 Date: Mar. 27, 2024

Related U.S. Application Data
(60) Provisional application No. 63/339,791, filed on May 9, 2022.
(60) Provisional application No. 63/250,818, filed on Sep. 30, 2021.

(51) Int. Cl. C07D 487/04; A61K 31/496; A61K 31/506; A61K 31/519; A61K 31/5377; C07D 471/04; C07D 519/00

(52) U.S. Cl. CPC C07D 487/04; A61K 31/496; A61K 31/506; A61K 31/519; A61K 31/5377; C07D 471/04; C07D 519/00

(57) ABSTRACT

The present disclosure relates to pharmaceutical compounds of the Formula (I), (Ia), (Ib), or (Ic) or a pharmaceutically acceptable salt or composition thereof. Also provided are methods of use of TRPML modulators for treating disorders, the modulators including compounds of the Formula (I), (Ia), (Ib), or (Ic). Such methods of use include treatment of ciliopathies.

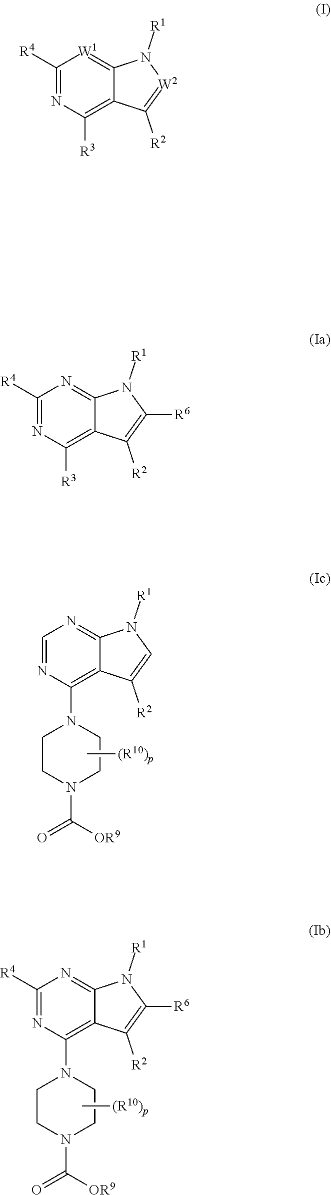

### CROSS-REFERENCE TO RELATED APPLICATIONS

The application claims the benefit of, and priority to, U.S. Ser. No. 63/250,818, filed Sep. 30, 2021; and U.S. Ser. No. 63/339,791, filed May 9, 2022; the contents of each of which are incorporated herein by reference in their entirety.

### TECHNICAL FIELD

The present disclosure relates to compounds and compositions which are modulators of TRPML and are useful for treatment of a variety of disorders.

### BACKGROUND

The lysosome is a key organelle that serves as the cell's recycling center. In a highly regulated manner, it breaks down various biomaterials (proteins, lipids, and membranes) into smaller molecules or chemical building blocks, that the cell then employs for energy or as starting materials for new proteins or membranes [see e.g., de Duve, C., The lysosome turns fifty. Nat Cell Biol, 2005. 7 (9): p. 847-9. Parkinson-Lawrence, E. J., et al., Lysosomal storage disease: revealing lysosomal function and physiology. Physiology (Bethesda), 2010. 25 (2): p. 102-15]. Lysosomal dysfunction due to mutations in the hydrolytic enzyme of lysosomal transport occur in the more than 50 genetically defined Lysosomal Storage Diseases. Interestingly, defects in lysosomal processing can have substantial effects on the function of the organelle beyond the actual enzyme that is mutated—in effect, the system can be gummed up—altering lysosomal degradation and membrane transport/trafficking, creating a positive feedback loop. Because lysosome storage is also seen in common neurodegenerative diseases such as Alzheimer's and Parkinson's, understanding the mechanisms underlying the positive feedback loop may provide therapeutic approaches not only for LSDs, but also for common sporadic neurodegenerative diseases. A lysosome-localized cation channel, TRPML1, has been recently identified as a key regulator of lysosomal function and membrane trafficking processes in the lysosome. Human mutations of TRPML1 cause an inherited lysosomal storage disease, Mucolipidosis IV. This disease is typified by neurodegenerative effects likely driven by the accumulation of lipids and other biomaterials in the cell. The related channels TRPML2 and TRPML3 also regulate lysosomal function.

Many reports suggest that TRPML channel activation is involved in multiple, key lysosomal functions. It can drive the translocation of the Transcription factor (TF)EB to the nucleus. TFEB regulates autophagy and lysosome biogenesis. Overexpression of TFEB has been reported to induce cellular clearance in several lysosome storage diseases, including Pompe Disease, Cystinosis, multiple sulfatase deficiency, as well as common neurodegenerative diseases, including Parkinson's disease and Huntington's disease (Settembre, C., et al., Signals from the lysosome: a control center for cellular clearance and energy metabolism. Nat Rev Mol Cell Biol, 2013. 14 (5): p. 283-96). Therefore, activation of TRPML channels by TRPML agonists may also lead to cellular clearance in all the aforementioned diseases, providing therapeutic targets for these devastating diseases.

Recently, a potent synthetic agonist for TRPML1 has been reported [Shen, D., et al., Lipid storage disorders block lysosomal trafficking by inhibiting a TRP channel and lysosomal calcium release. Nat Commun, 2012. 3: p. 731]. This SF-51-related compound (Mucolipin Synthetic Agonist 1 or ML-SA1) that could induce significant [Ca2+] increases in HEK293 cells stably or transiently expressing TRPML1 protein that has been forced to the plasma membrane via deletion of its lysosomal targeting sequence. High concentrations of ML-SA1 (˜10 μM) are needed to effectively activate TRPMLs. Since that concentration is usually difficult to achieve in vivo, ML-SA1 cannot be used to treat the above TRPML related diseases. Liang et al. recently reported a new class of compounds as more potent TRPML activators [WO 2018/005713A1]. These compounds were thought to be useful in treating disorders related to TRPML activities such as lysosome storage diseases, muscular dystrophy, age-related common neurodegenerative diseases, ROS or oxidative stress related diseases, and aging. TRPML activators may also be useful in other disorders.

### SUMMARY OF THE DISCLOSURE

The present disclosure provides for a compound of Formula (I) and subformulas thereof and pharmaceutically acceptable salts, solvates, hydrates, tautomers, and stereoisomers thereof.

In an aspect, provided is a compound of Formula (I)

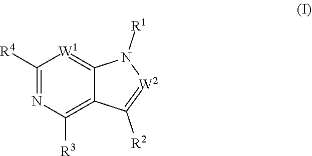

or a pharmaceutically acceptable salt thereof, wherein
W¹ is N or CR⁵;
W² is N or CR⁶;
R¹ is aryl, heteroaryl, cycloalkyl, or heterocycloalkyl, each R¹ optionally substituted by 1-5 independently selected R⁷;
R² is aryl, heteroaryl, cycloalkyl, heterocycloalkyl, —O-cycloalkyl, —O-heterocycloalkyl, C₁₋₆ alkyl, C₁₋₆ alkoxy, —(CH₂)₁₋₂-cycloalkyl, —(CH₂)₁₋₂-heterocycloalkyl, or NR^{a}R^{b}, each R² optionally substituted by 1-5 independently selected R⁸;

### R³ is

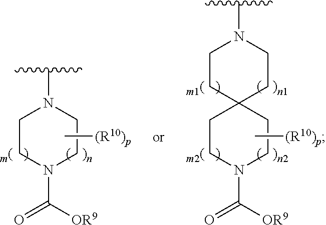

each of R⁴, R⁵, and R⁶ is independently selected from the group consisting of H, hydroxy, halogen, cyano, C₁₋₆ alkyl, C₁₋₆ haloalkyl, C₁₋₆ alkoxy, C₁₋₆ haloalkoxy, C₁₋₆ alkylthio, and NR^{a}R^{b};
each of R⁷ and R⁸ are independently selected at each occurrence from the group consisting of deuterium, hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl; or
when R¹ or R² is cycloalkyl or heterocycloalkyl, two R⁷ or two R⁸ on the same carbon can be taken together to form oxo, or any two R⁷ or two R⁸ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
R⁹ is C₁₋₆ alkyl or —(CH₂)₀₋₂—C₃₋₇ cycloalkyl, optionally substituted with substituents independently selected from the group consisting of deuterium, halogen, hydroxyl, C₁₋₃ alkyl, and C₁₋₆ alkoxy, wherein C₁₋₃ alkyl and C₁₋₆ alkoxy are optionally substituted with 1-5 substituents independently selected from the group consisting of deuterium, hydroxyl, C₁₋₃ haloalkyl, C₁₋₃ alkoxy and C₁₋₃ alkyl;
each R¹⁰ is selected independently from the group consisting of C₁₋₆ alkyl and C₁₋₆ haloalkyl, optionally substituted with substituents independently selected from the group consisting of deuterium, hydroxyl, and C₁₋₆ alkoxy; or
   two R¹⁰ on the same carbon can be taken together to form oxo; or
   any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of deuterium, halogen, hydroxyl, C₁₋₆ haloalkyl, and C₁₋₆ alkyl;
   each R^{a} and R^{b} is independently selected from H, C₁₋₆ alkyl, C(O)—O—C₁₋₆ alkyl, C(O)—O—C₂₋₆ alkenyl, C(O)—C₁₋₆ alkyl, C(O)—C₂₋₆ alkenyl, —(CH₂)₀₋₂—C₃₋₇ cycloalkyl, and 3-7 membered heterocycloalkyl, wherein each alkyl, cycloalkyl or heterocycloalkyl is optionally substituted by 1-3 substituents selected from halogen and C₁₋₆ alkoxy; or
   R^{a} and R^{b} can be taken together with the nitrogen to which they are attached to form a 4-7 membered ring;
   m is 1 or 2;
   n is 1, 2, or 3;
   m1 is 0, 1 or 2;
   n1 is 0, 1, 2, or 3;
   m2 is 0, 1 or 2;
   n2 is 0, 1, 2, or 3;
   p is 0, 1, 2, 3, 4, 5, 6, 7, or 8;
   wherein m+n is 2, 3, or 4;
   m1+n1 is 0, 1, 2, 3, or 4;
   m2+n2 is 0, 1, 2, 3, or 4; and
   R¹ and R² are not both aryl.

In another aspect, provided is a compound of formula (Ib)

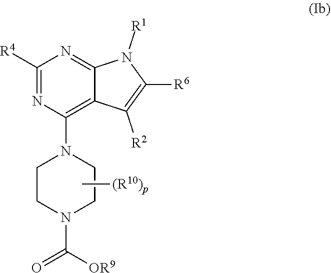

wherein the variable definitions are as described in the specification and claims.

In another aspect, provided is a compound of formula (Ic)

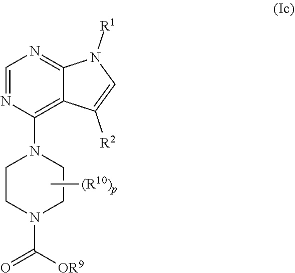

wherein the variable definitions are as described in the specification and claims.

In another aspect, provided is a compound of Formula (I), or any subformula thereof, selected from the compounds disclosed in the specification or claims, or a pharmaceutically acceptable salt thereof.

In another aspect, the disclosure provides a method of treating a disease or disorder that can be treated by modulation of TRPML, the method comprising administering to a patient in need thereof a compound described herein, or a composition described herein.

Still other objects and advantages of the disclosure will become apparent to those of skill in the art from the disclosure herein, which is simply illustrative and not restrictive. Thus, other embodiments will be recognized by the skilled artisan without departing from the spirit and scope of the disclosure.

### DETAILED DESCRIPTION OF THE DISCLOSURE

As generally described herein, the present disclosure provides compounds (e.g., compounds of Formula (I), and its subformulas (Ia), (Ib), and (Ic), or compounds of Table 1, or pharmaceutically acceptable salts thereof) that are useful for disorders (e.g., polycystic kidney disease) associated with modulation of TRPML. “TRPML”, “TRPML ion channel” and “TRPML channel” are used interchangeably throughout.

### Compounds

In an aspect, provided is a compound of Formula (I):

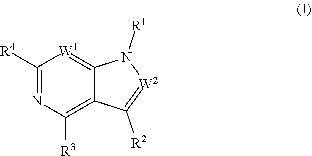

or a pharmaceutically acceptable salt thereof, wherein
W¹ is N or CR⁵;
W² is N or CR⁶;
R¹ is aryl, heteroaryl, cycloalkyl, or heterocycloalkyl, each R¹ optionally substituted by 1-5 independently selected R⁷;
R² is aryl, heteroaryl, cycloalkyl, heterocycloalkyl, —O-cycloalkyl, —O-heterocycloalkyl, C₁₋₆ alkyl, C₁₋₆ alkoxy, —(CH₂)₁₋₂-cycloalkyl, —(CH₂)₁₋₂-heterocycloalkyl, or NR^{a}R^{b}, each R² optionally substituted by 1-5 independently selected R⁸;

### R³ is

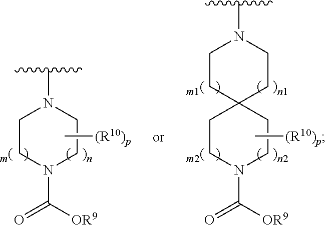

each of R⁴, R⁵, and R⁶ is independently selected from the group consisting of H, hydroxy, halogen, cyano, C₁₋₆ alkyl, C₁₋₆ haloalkyl, C₁₋₆ alkoxy, C₁₋₆ haloalkoxy, C₁₋₆ alkylthio, and NR^{a}R^{b};
each of R⁷ and R⁸ are independently selected at each occurrence from the group consisting of deuterium, hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl; or
when R¹ or R² is cycloalkyl or heterocycloalkyl, two R⁷ or two R⁸ on the same carbon can be taken together to form oxo, or any two R⁷ or two R⁸ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
R⁹ is C₁₋₆ alkyl or —(CH₂)₀₋₂—C₃₋₇ cycloalkyl, optionally substituted with substituents independently selected from the group consisting of deuterium, halogen, hydroxyl, C₁₋₃ alkyl, and C₁₋₆ alkoxy, wherein C₁₋₃ alkyl and C₁₋₆ alkoxy are optionally substituted with 1-5 substituents independently selected from the group consisting of deuterium, hydroxyl, C₁₋₃ haloalkyl, C₁₋₃ alkoxy and C₁₋₃ alkyl;
each R¹⁰ is selected independently from the group consisting of C₁₋₆ alkyl and C₁₋₆ haloalkyl, optionally substituted with substituents independently selected from the group consisting of deuterium, hydroxyl, and C₁₋₆ alkoxy; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of deuterium, halogen, hydroxyl, C₁₋₆ haloalkyl, and C₁₋₆ alkyl;
each R^{a} and R^{b} is independently selected from H, C₁₋₆ alkyl, C(O)—O—C₁₋₆ alkyl, C(O)—O—C₂₋₆ alkenyl, C(O)—C₁₋₆ alkyl, C(O)—C₂₋₆ alkenyl, —(CH₂)₀₋₂—C₃₋₇ cycloalkyl, and 3-7 membered heterocycloalkyl, wherein each alkyl, cycloalkyl or heterocycloalkyl is optionally substituted by 1-3 substituents selected from halogen and C₁₋₆ alkoxy; or
R^{a} and R^{b} can be taken together with the nitrogen to which they are attached to form a 4-7 membered ring;
m is 1 or 2;
n is 1, 2, or 3;
m1 is 0, 1 or 2;
n1 is 0, 1, 2, or 3;
m2 is 0, 1 or 2;
n2 is 0, 1, 2, or 3;
p is 0, 1, 2, 3, 4, 5, 6, 7, or 8;
wherein m+n is 2, 3, or 4;
m1+n1 is 0, 1, 2, 3, or 4;
m2+n2 is 0, 1, 2, 3, or 4; and
R¹ and R² are not both aryl.

In some embodiments, W¹ is N. In some embodiments, W² is CR⁶. In some embodiments, W¹ is N and W² is CR⁶. In some embodiments, W¹ is CR⁵ and W² is N. In some embodiments, W¹ is N and W² is N. In some embodiments, W¹ is CR⁵ and W² is CR⁶.

In some embodiments, the compound is of formula (Ia)

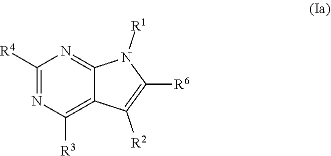

or a pharmaceutically acceptable salt thereof, wherein
R¹ is aryl, heteroaryl, cycloalkyl, or heterocycloalkyl, each R¹ optionally substituted by 1-5 independently selected R⁷;
R² is aryl, heteroaryl, cycloalkyl, heterocycloalkyl, —O-cycloalkyl, C₁₋₆ alkyl, C₁₋₆ alkoxy, or NR^{a}R^{b}, each R² optionally substituted by 1-5 independently selected R⁸;

### R³ is

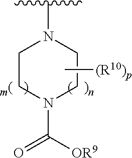

each of R⁴ and R⁶ is independently selected from the group consisting of H, hydroxy, halogen, cyano, C₁₋₆ alkyl, C₁₋₆ haloalkyl, C₁₋₆ alkoxy, C₁₋₆ haloalkoxy, C₁₋₆ alkylthio, and NR^{a}R^{b};
each of R⁷ and R⁸ are independently selected at each occurrence from the group consisting of deuterium, hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl; or
when R¹ or R² is cycloalkyl or heterocycloalkyl, two R⁷ or two R⁸ on the same carbon can be taken together to form oxo, or any two R⁷ or two R⁸ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
R⁹ is C₁₋₆ alkyl, optionally substituted with substituents independently selected from the group consisting of deuterium, halogen, hydroxyl, and C₁₋₆ alkoxy;
each R¹⁰ is selected independently from the group consisting of C₁₋₆ alkyl and C₁₋₆ haloalkyl, optionally substituted with substituents independently selected from the group consisting of deuterium, hydroxyl, and C₁₋₆ alkoxy; or
two R¹⁰ on the same carbon can be taken together to form oxo; or any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
each R^{a} and R^{b} is independently selected from H, C₁₋₆ alkyl, C(O)—O—C₁₋₆ alkyl, C(O)—O—C₂₋₆ alkenyl, —(CH₂)₀₋₂—C₃₋₇ cycloalkyl, and 3-7 membered heterocycloalkyl, wherein each alkyl, cycloalkyl or heterocycloalkyl is optionally substituted by 1-3 substituents selected from halogen and C₁₋₆ alkoxy; or
R^{a} and R^{b} can be taken together with the nitrogen to which they are attached to form a 4-7 membered ring;
m is 1 or 2;
n is 1, 2, or 3;
p is 0, 1, 2, 3, 4, 5, 6, 7, or 8;
wherein m+n is 2, 3, or 4; and
R¹ and R² are not both aryl.

In some embodiments, the compound is a compound of formula (Ia)

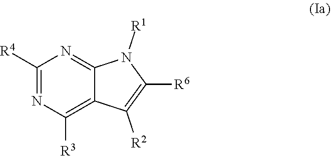

or a pharmaceutically acceptable salt thereof, wherein
R¹ is aryl, heteroaryl, cycloalkyl, or heterocycloalkyl, each R¹ optionally substituted by 1-5 independently selected R⁷;
R² is C₁₋₆ alkyl or C₃₋₇ cycloalkyl, optionally substituted by 1-5 independently selected R⁸;

### R³ is

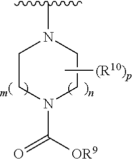

each of R⁴ and R⁶ is independently selected from the group consisting of H, hydroxy, halogen, cyano, C₁₋₆ alkyl, C₁₋₆ haloalkyl, C₁₋₆ alkoxy, C₁₋₆ haloalkoxy, C₁₋₆ alkylthio, and NR^{a}R^{b};
each of R⁷ and R⁸ are independently selected at each occurrence from the group consisting of deuterium, hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl; or
when R¹ or R² is cycloalkyl or heterocycloalkyl, two R⁷ or two R⁸ on the same carbon can be taken together to form oxo, or any two R⁷ or two R⁸ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
R⁹ is C₁₋₆ alkyl, optionally substituted with substituents independently selected from the group consisting of deuterium, halogen, hydroxyl, and C₁₋₆ alkoxy;
each R¹⁰ is selected independently from the group consisting of C₁₋₆ alkyl and C₁₋₆ haloalkyl, optionally substituted with substituents independently selected from the group consisting of deuterium, hydroxyl, and C₁₋₆ alkoxy; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
each R^{a} and R^{b} is independently selected from H, C₁₋₆ alkyl, C(O)—O—C₁₋₆ alkyl, C(O)—O—C₂₋₆ alkenyl, —(CH₂)₀₋₂—C₃₋₇ cycloalkyl, and 3-7 membered heterocycloalkyl, wherein each alkyl, cycloalkyl or heterocycloalkyl is optionally substituted by 1-3 substituents selected from halogen and C₁₋₆ alkoxy; or
R^{a} and R^{b} can be taken together with the nitrogen to which they are attached to form a 4-7 membered ring;
m is 1 or 2;
n is 1, 2, or 3;
p is 0, 1, 2, 3, 4, 5, 6, 7, or 8; and
wherein m+n is 2, 3, or 4.

In some embodiments, the compound is a compound of formula (Ia)

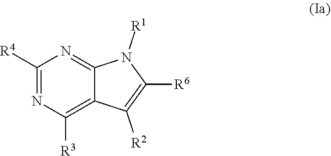

or a pharmaceutically acceptable salt thereof, wherein
R¹ is aryl, heteroaryl, cycloalkyl, or heterocycloalkyl, each R¹ optionally substituted by 1-5 independently selected R⁷;
R² is C₁₋₆ alkyl, optionally substituted by 1-5 independently selected R⁸;

### R³ is

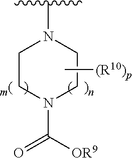

each of R⁴ and R⁶ is independently selected from the group consisting of H, hydroxy, halogen, cyano, C₁₋₆ alkyl, C₁₋₆ haloalkyl, C₁₋₆ alkoxy, C₁₋₆ haloalkoxy, C₁₋₆ alkylthio, and NR^{a}R^{b};
each of R⁷ and R⁸ are independently selected at each occurrence from the group consisting of deuterium, hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl; or
when R¹ or R² is cycloalkyl or heterocycloalkyl, two R⁷ or two R⁸ on the same carbon can be taken together to form oxo, or any two R⁷ or two R⁸ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
R⁹ is C₁₋₆ alkyl, optionally substituted with substituents independently selected from the group consisting of deuterium, halogen, hydroxyl, and C₁₋₆ alkoxy;
each R¹⁰ is selected independently from the group consisting of C₁₋₆ alkyl and C₁₋₆ haloalkyl, optionally substituted with substituents independently selected from the group consisting of deuterium, hydroxyl, and C₁₋₆ alkoxy; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
each R^{a} and R^{b} is independently selected from H, C₁₋₆ alkyl, C(O)—O—C₁₋₆ alkyl, C(O)—O—C₂₋₆ alkenyl, —(CH₂)₀₋₂—C₃₋₇ cycloalkyl, and 3-7 membered heterocycloalkyl, wherein each alkyl, cycloalkyl or heterocycloalkyl is optionally substituted by 1-3 substituents selected from halogen and C₁₋₆ alkoxy; or
R^{a} and R^{b} can be taken together with the nitrogen to which they are attached to form a 4-7 membered ring;
m is 1 or 2;
n is 1, 2, or 3;
p is 0, 1, 2, 3, 4, 5, 6, 7, or 8; and
wherein m+n is 2, 3, or 4.

In some embodiments, the compound is a compound of formula (Ia)

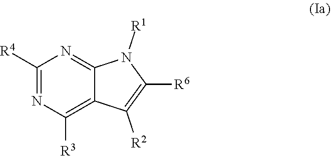

or a pharmaceutically acceptable salt thereof, wherein
R¹ is heteroaryl optionally substituted by 1-5 independently selected R⁷;
R² is C₁₋₆ alkyl optionally substituted by 1-5 independently selected R⁸;

### R³ is

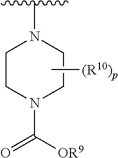

each of R⁴ and R⁶ is H;
each of R⁷ and R⁸ are independently selected at each occurrence from the group consisting of deuterium, hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl;
R⁹ is C₁₋₆ alkyl, optionally substituted with substituents independently selected from the group consisting of deuterium, halogen, hydroxyl, and C₁₋₆ alkoxy;
each R¹⁰ is selected independently from the group consisting of C₁₋₆ alkyl and C₁₋₆ haloalkyl, optionally substituted with substituents independently selected from the group consisting of deuterium, hydroxyl, and C₁₋₆ alkoxy; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
each R^{a} and R^{b} is independently selected from H, C₁₋₆ alkyl, C(O)—O—C₁₋₆ alkyl, C(O)—O—C₂₋₆ alkenyl, —(CH₂)₀₋₂—C₃₋₇ cycloalkyl, and 3-7 membered heterocycloalkyl, wherein each alkyl, cycloalkyl or heterocycloalkyl is optionally substituted by 1-3 substituents selected from halogen and C₁₋₆ alkoxy; or
R^{a} and R^{b} can be taken together with the nitrogen to which they are attached to form a 4-7 membered ring; and
p is 0, 1, 2, 3, 4, 5, 6, 7, or 8.

In some embodiments, the compound is a compound of formula (Ia)

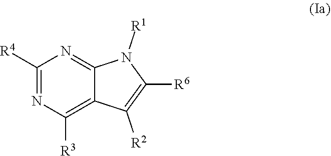

or a pharmaceutically acceptable salt thereof, wherein
R¹ is pyridyl optionally substituted by 1-5 independently selected R⁷;
R² is C₁₋₆ alkyl optionally substituted by 1-5 independently selected R⁸;

### R³ is

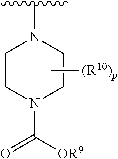

each of R⁴ and R⁶ is H;
each of R⁷ and R⁸ are independently selected at each occurrence from the group consisting of deuterium, hydroxy, halogen, cyano, C₁₋₆ alkyl, and C₁₋₆ alkoxy;
R⁹ is C₁₋₆ alkyl, optionally substituted with substituents independently selected from the group consisting of deuterium, halogen, hydroxyl, and C₁₋₆ alkoxy;
each R¹⁰ is selected independently from the group consisting of C₁₋₆ alkyl, and C₁₋₆ haloalkyl, each optionally substituted with 1-5 deuterium; and
p is 0, 1, 2, 3, 4, 5, 6, 7, or 8.

In some embodiments, R¹ is aryl optionally substituted by 1-5 independently selected R⁷. In some embodiments, R¹ is phenyl optionally substituted with 1-3 independently selected R⁷.

In some embodiments, each R⁷ is independently selected from H, halogen, cyano, C₁₋₆ alkyl, C₁₋₆ alkoxy, C₁₋₆ haloalkyl, or C₁₋₆ haloalkoxy.

In some embodiments, R¹ is heteroaryl, cycloalkyl, or heterocycloalkyl, each R¹ optionally substituted by 1-5 independently selected R⁷.

In some embodiments, R¹ is heteroaryl or heterocycloalkyl, each R¹ optionally substituted by 1-5 independently selected R⁷. In some embodiments, R¹ is heteroaryl optionally substituted by 1-5 independently selected R⁷. In some embodiments, R¹ is monocyclic heteroaryl optionally substituted by 1-5 independently selected R⁷. In some embodiments, R¹ is monocyclic heteroaryl of 5-6 ring atoms with 1, 2 or 3 ring atoms selected independently from N, O, and S, wherein R¹ is optionally substituted by 1-4 independently selected R⁷. In some embodiments, R¹ is monocyclic nitrogen-containing heteroaryl of 5-6 ring atoms with 1, 2 or 3 ring atoms selected independently from N, O, and S, wherein R¹ is optionally substituted by 1-4 independently selected R⁷. In some embodiments, R¹ is monocyclic nitrogen-containing heteroaryl of 5-6 ring atoms with 1, 2 or 3 ring heteroatoms selected from N only, wherein R¹ is optionally substituted by 1-4 independently selected R⁷. In some embodiments, R¹ is pyridine, pyrimidine, pyrazine, pyridazine, thiazole, oxazole, pyrrole, imidazole, or pyrazole, optionally substituted by 1-4 independently selected R⁷. In some embodiments, R¹ is pyridine, thiazole, or pyrazole, optionally substituted by 1-4 independently selected R⁷. In some embodiments, R¹ is pyridine, optionally substituted by 1-4 independently selected R⁷. In some embodiments, R¹ is 2-pyridyl, optionally substituted by 1-4 independently selected R⁷.

In some embodiments, R¹ is

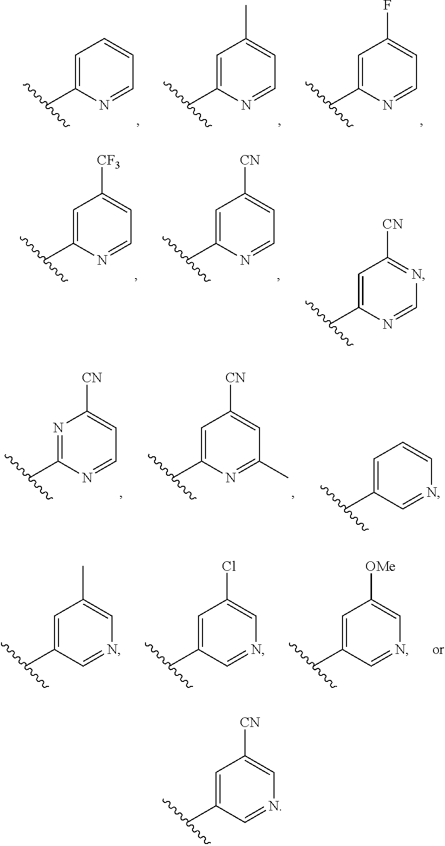

In some embodiments, R¹ is

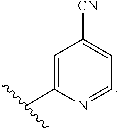 [SMILES: *c1cc(C#N)ccn1]

In some embodiments, R¹ is

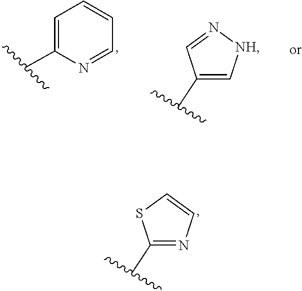 [SMILES: *c1ccccn1 ; *c1cn[nH]c1 ; *c1nccs1]

optionally substituted by 1-4 independently selected R⁷.

In some embodiments, R¹ is heterocycloalkyl of 4-8 ring atoms, wherein 1-3 ring atoms are selected from N, O, and S, and R¹ is optionally substituted by 1-4 independently selected R⁷. In some embodiments, R¹ is monocyclic heterocycloalkyl of 4-7 ring atoms, wherein 1-3 ring atoms are selected from N, O, and S, and R¹ is optionally substituted by 1-4 independently selected R⁷. In some embodiments, R¹ is tetrahydropyran, azetidine, pyrrolidine, morpholine, or piperidine, and R¹ is optionally substituted by 1-4 independently selected R⁷.

In some embodiments, R¹ is C₃₋₇ cycloalkyl, optionally substituted by 1-4 independently selected R⁷. In some embodiments, R¹ is a cyclohexyl with an optional one or two carbon bridged ring, and R¹ is optionally substituted by 1-4 independently selected R⁷.

In some embodiments, R² is aryl, heteroaryl, cycloalkyl, heterocycloalkyl, —O— cycloalkyl, —O-heterocycloalkyl, C₁₋₆ alkoxy, C₁₋₆ alkyl, or NR^{a}R^{b}, each R² optionally substituted by 1-5 independently selected R⁸, and wherein R^{a} and R^{b} of the R² group are not both H. In some embodiments, R² is aryl, heteroaryl, cycloalkyl, or heterocycloalkyl, each R² optionally substituted by 1-5 independently selected R⁸. In some embodiments, R² is aryl optionally substituted by 1-5 independently selected R⁸. In some embodiments, R² is phenyl optionally substituted with 1-3 independently selected R⁸.

In some embodiments, each R⁸ is independently selected from H, halogen, cyano, C₁₋₆ alkyl, C₁₋₆ alkoxy, C₁₋₆ haloalkyl, or C₁₋₆ haloalkoxy.

In some embodiments, R² is

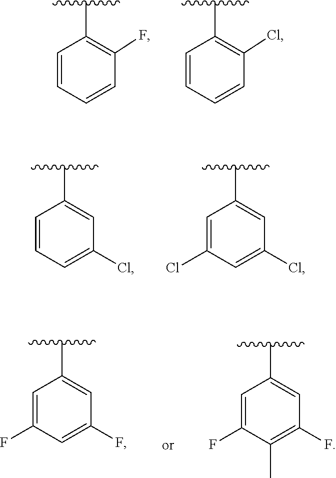

In some embodiments, R² is heteroaryl, cycloalkyl, or heterocycloalkyl, each R² optionally substituted by 1-5 independently selected R⁸. In some embodiments, R² is heteroaryl or heterocycloalkyl, each R² optionally substituted by 1-5 independently selected R⁸. In some embodiments, R² is heteroaryl optionally substituted by 1-5 independently selected R⁸. In some embodiments, R² is monocyclic heteroaryl optionally substituted by 1-5 independently selected R⁸. In some embodiments, R² is monocyclic heteroaryl of 5-6 ring atoms with 1, 2 or 3 ring atoms selected independently from N, O, and S, wherein R² is optionally substituted by 1-4 independently selected R⁸. In some embodiments, R² is pyridine, pyrimidine, pyrazine, pyridazine, thiazole, oxazole, pyrrole, imidazole, or pyrazole, optionally substituted by 1-4 independently selected R⁸. In some embodiments, R² is pyridine, pyrimidine, pyrazine, or pyrazole, optionally substituted by 1-4 independently selected R⁸. In some embodiments, R² is pyridine, optionally substituted by 1-4 independently selected R⁸.

In some embodiments, R² is

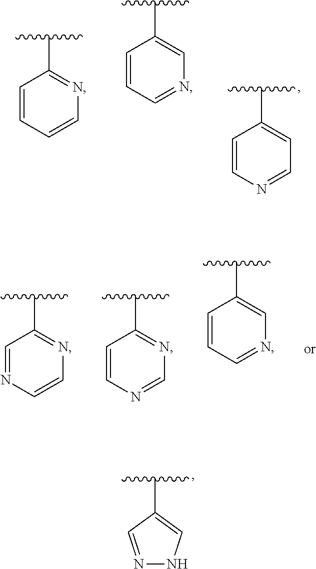

optionally substituted by 1-4 independently selected R⁸.

In some embodiments, R² is

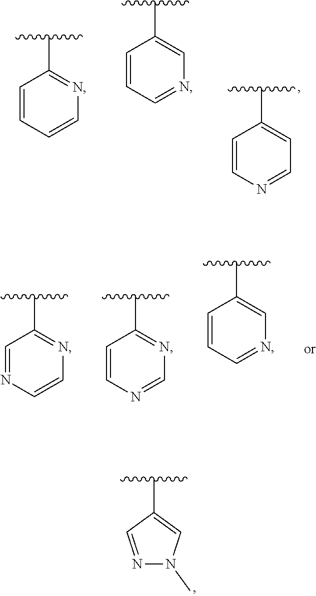

and wherein R² is not further substituted.

In some embodiments, R² is

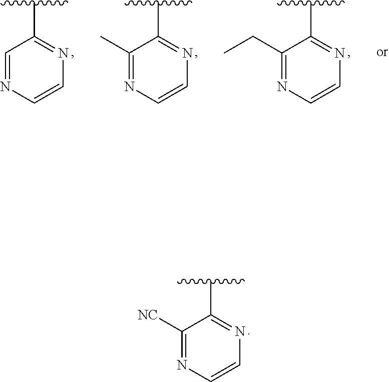

In some embodiments, R² is cycloalkyl optionally substituted by 1-5 independently selected R⁸. In some embodiments, R² is C₃₋₈ cycloalkyl optionally substituted with 1-5 independently selected R⁸. In some embodiments, R² is cyclopropyl, cyclobutyl, cyclopentyl, or cyclohexyl, each optionally substituted with 1-3 independently selected R⁸. In some embodiments, R² is not substituted. In some embodiments, R² is cyclopropyl.

In some embodiments, R² is heterocycloalkyl optionally substituted by 1-5 independently selected R⁸. In some embodiments, R² is monocyclic heterocycloalkyl optionally substituted by 1-5 independently selected R⁸. In some embodiments, R² is monocyclic heterocycloalkyl of 4-6 ring atoms with 1, 2 or 3 ring atoms selected independently from N, O, and S, wherein R² is optionally substituted by 1-4 independently selected R⁸. In some embodiments, R² is azetidine, oxetane, pyrrolidine, tetrahydrofuran, piperidine, piperazine, tetrahydropyran, or morpholine, optionally substituted by 1-4 independently selected R⁸. In some embodiments R² is azetidine, pyrrolidine, piperazine, or morpholine, optionally substituted by 1-4 independently selected R⁸. In some embodiments, R² contains a ring nitrogen atom and is bound to formula (I) at the ring nitrogen atom.

In some embodiments, R² is

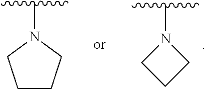 [SMILES: *N1CCCC1 ; *N1CCC1]

In some embodiments, R² is NR^{a}R^{b}, each R² optionally substituted by 1-5 independently selected R⁸, and wherein R^{a} and R^{b} of the R² group are not both H. In some embodiments, R^{a} is C₁₋₆ alkyl, and R^{b} is C₁₋₆ alkyl, C₃₋₇ cycloalkyl, or 3-7 membered heterocycloalkyl. In some embodiments, R^{a} and R^{b} are each independently selected C₁₋₆ alkyl.

In some embodiments, each R⁸ is independently selected from deuterium, hydroxy, halogen, cyano, or C₁₋₆ alkoxy.

In some embodiments, R² is C₁₋₆ alkyl optionally substituted by 1-5 independently selected R⁸. In some embodiments, R² is C₁₋₆ alkyl optionally substituted by 1-5 independently selected halogens. In some embodiments, R² is Me, Et, CHF₂, or CF₃. In some embodiments, R² is CHF₂. In some embodiments, R² is CF₃.

In some embodiments, R² is not substituted.

In some embodiments, R² is O-cycloalkyl, —O-heterocycloalkyl, or C₁₋₆ alkoxy, each R² optionally substituted by 1-5 independently selected R⁸. In some embodiments, R² is —O—C₃₋₇ cycloalkyl or C₁₋₆ alkoxy, each R² optionally substituted by 1-5 groups independently selected from alkyl and halogen. In some embodiments, R² is —O—C₃₋₇ cycloalkyl or C₁₋₆ alkoxy, each R² optionally substituted by 1-5 independently selected halogens. In some embodiments, R² is —O-cyclobutyl, —O-propyl, —O-methyl, —OCHF₂, or —O—CF₃.

In some embodiments, m is 1 and n is 1.

In some embodiments, p is 0, 1, 2, 3, 4, 5, or 6.

In some embodiments, each R¹⁰ is independently selected from the group consisting of C₁₋₆ alkyl and C₁₋₆ haloalkyl, each optionally substituted with 1-5 deuteriums. In some embodiments, each R¹⁰ is methyl.

In some embodiments, p is 1, 2, 3, 4, 5, or 6.

In some embodiments, R³ is substituted with an edge fused or spiro fused cyclopropane; or R³ includes a one or two carbon bridge or a single bond bridge; and R³ is optionally additionally substituted by 1-4 R¹⁰.

In some embodiments, R³ is

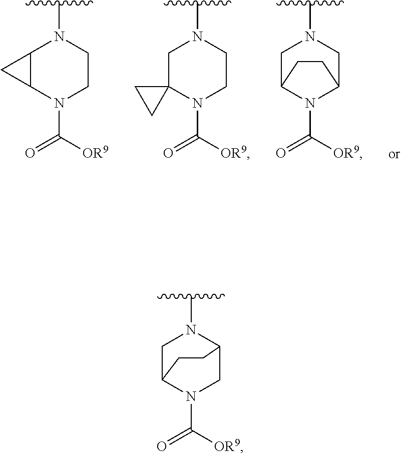

and R³ is optionally additionally substituted by 1-4 R¹⁰.

In some embodiments, R³ is

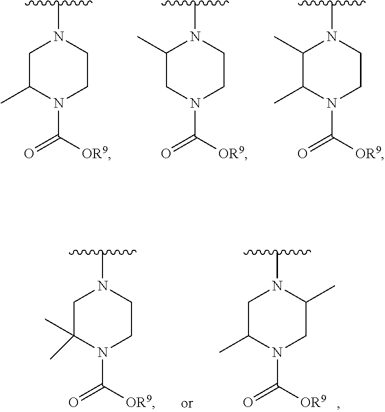

and R³ is optionally additionally substituted by 1-4 R¹⁰.

In some embodiments, R³ is

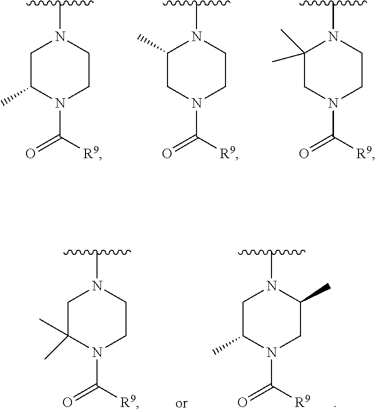

In some embodiments, R³ is not substituted by any additional R¹⁰.

In some embodiments, R⁴ is H. In some embodiments, R⁵ is H. In some embodiments, R⁶ is H. In some embodiments, R⁴ and R⁶ are H. In some embodiments, R⁴, R⁵, and R⁶ are H.

In some embodiments, each of R⁷ and R⁸ are independently selected at each occurrence from the group consisting of hydroxy, halogen, cyano, C₁₋₆ alkyl, C₁₋₆ alkoxy, and C₃₋₇ cycloalkyl, wherein each C₁₋₆ alkyl and C₁₋₆ alkoxy is optionally substituted with 1-3 halogens. In some embodiments, each R⁷ is independently selected at each occurrence from the group consisting of halogen, cyano, C₁₋₆ alkyl, C₁₋₆ alkoxy, and CF₃.

In some embodiments, R⁹ is C₁₋₆ alkyl optionally substituted with 1-5 halogens or 1-9 deuteriums. In some embodiments, R⁹ is ethyl, isopropyl, or t-butyl; each optionally substituted with 1-5 halogens or 1-9 deuteriums. In some embodiments, R⁹ is ethyl, isopropyl, or t-butyl. In some embodiments, R⁹ is t-butyl. In some embodiments, R⁹ is —C(CD₃)₃, —CH(CD₃)₂, or —CD₂CD₃.

In some embodiments, R⁹ is Me, Et, t-butyl, —C(CD₃)₃, —CH(CD₃)₂, —C(CD₃)₃, isopropyl, F₃C—CH₂—, F₃C—CH(CH₃)—, F₃C—C(CH₃)₂—, or FCH₂—C(CH₃)₂—,

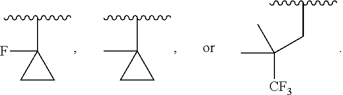

In some embodiments, R⁹ is Me, Et, t-butyl, —C(CD₃)₃, —CH(CD₃)₂, —C(CD₃)₃, isopropyl, F₃C—CH₂—, F₃C—CH(CH₃)—, F₃C—C(CH₃)₂—, or FCH₂—C(CH₃)₂—, or

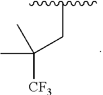 [SMILES: *CC(C)(C)C(F)(F)F]

In another aspect, provided is a compound of formula (Ib)

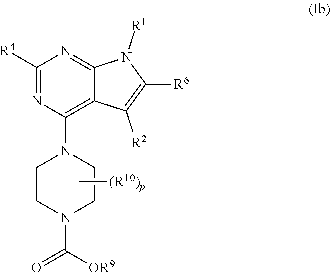

wherein the variable definitions are as described in the specification and claims.

In another aspect, provided is a compound of formula (Ic)

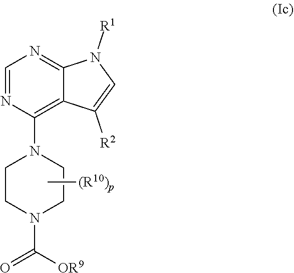

wherein the variable definitions are as described in the specification and claims.

In another aspect, provided is a compound of Formula (I), or any subformula thereof, selected from the compounds disclosed in the specification or claims, or a pharmaceutically acceptable salt thereof.

In some embodiments, the compound achieves at least 50% of the maximal current obtained with 30 μM ML-SA1 in a patch clamp assay for a TRPML and has an EC₅₀ less than 1 μM. In some embodiments, the compound achieves at least 50% of the maximal current obtained with 30 μM ML-SA1 in a patch clamp assay for TRPML1 and has an EC₅₀ less than 1 μM. In some embodiments, the compound achieves a maximal current obtained with 30 μM ML-SA1 in a patch clamp assay for TRPML1 which is at least 10 fold the maximal current achieved for any other TRPML.

In another aspect pharmaceutical composition comprising a pharmaceutically acceptable excipient and a compound selected from the compounds disclosed in the specification or claims, or a pharmaceutically acceptable salt thereof. In some embodiments, the compound is a compound identified in Table 1 below or a pharmaceutically acceptable salt thereof.

TABLE 1
Exemplary compounds

| Cmpd | |
|---|---|
| No. | Structure |
| 101 | 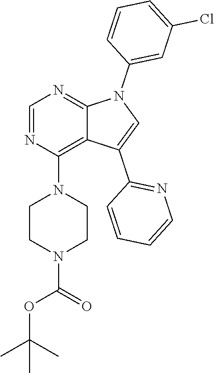 [SMILES: CC(C)(C)OC(=O)N1CCN(c2ncnc3c2c(-c2ccccn2)cn3-c2cccc(Cl)c2)CC1] |
| 102 | 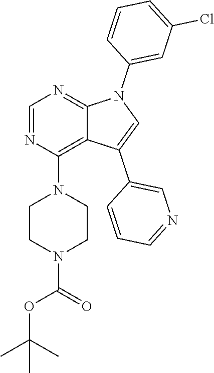 [SMILES: CC(C)(C)OC(=O)N1CCN(c2ncnc3c2c(-c2cccnc2)cn3-c2cccc(Cl)c2)CC1] |
| 103 | 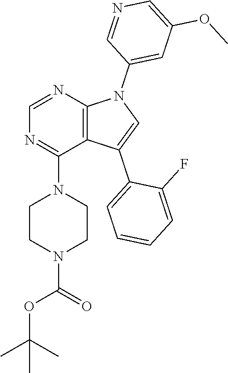 [SMILES: COc1cncc(-n2cc(-c3ccccc3F)c3c(N4CCN(C(=O)OC(C)(C)C)CC4)ncnc32)c1] |
| 104 | 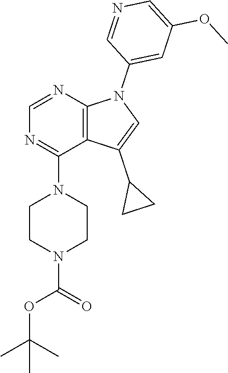 [SMILES: COc1cncc(-n2cc(C3CC3)c3c(N4CCN(C(=O)OC(C)(C)C)CC4)ncnc32)c1] |
| 105 | 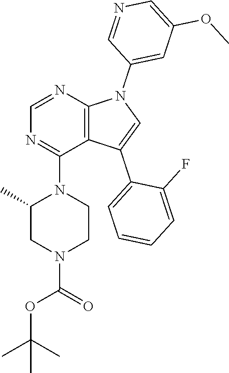 [SMILES: COc1cncc(-n2cc(-c3ccccc3F)c3c(N4CCN(C(=O)OC(C)(C)C)C[C@@H]4C)ncnc32)c1] |
| 106 | 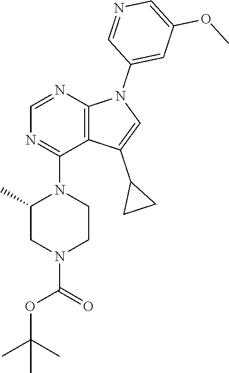 [SMILES: COc1cncc(-n2cc(C3CC3)c3c(N4CCN(C(=O)OC(C)(C)C)C[C@@H]4C)ncnc32)c1] |
| 107 | 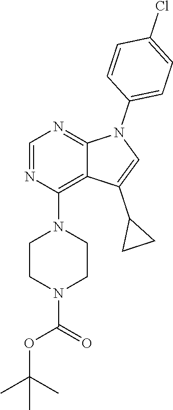 [SMILES: CC(C)(C)OC(=O)N1CCN(c2ncnc3c2c(C2CC2)cn3-c2ccc(Cl)cc2)CC1] |
| 108 | 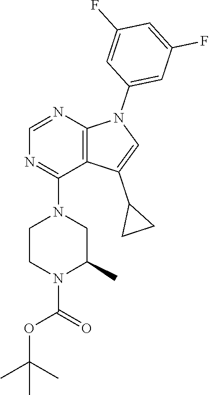 [SMILES: C[C@@H]1CN(c2ncnc3c2c(C2CC2)cn3-c2cc(F)cc(F)c2)CCN1C(=O)OC(C)(C)C] |
| 109 | 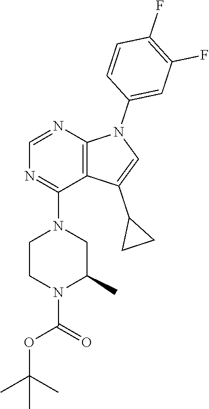 [SMILES: C[C@@H]1CN(c2ncnc3c2c(C2CC2)cn3-c2ccc(F)c(F)c2)CCN1C(=O)OC(C)(C)C] |
| 110 | 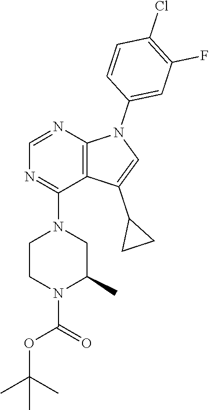 [SMILES: C[C@@H]1CN(c2ncnc3c2c(C2CC2)cn3-c2ccc(Cl)c(F)c2)CCN1C(=O)OC(C)(C)C] |
| 111 | 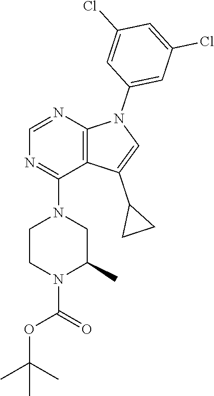 [SMILES: C[C@@H]1CN(c2ncnc3c2c(C2CC2)cn3-c2cc(Cl)cc(Cl)c2)CCN1C(=O)OC(C)(C)C] |
| 112 | 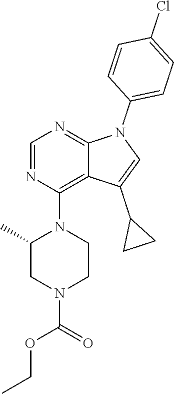 [SMILES: CCOC(=O)N1CCN(c2ncnc3c2c(C2CC2)cn3-c2ccc(Cl)cc2)[C@@H](C)C1] |
| 113 | 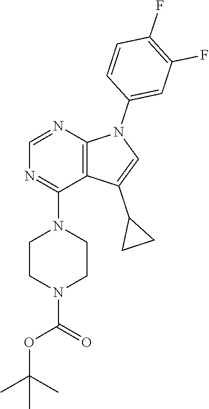 [SMILES: CC(C)(C)OC(=O)N1CCN(c2ncnc3c2c(C2CC2)cn3-c2ccc(F)c(F)c2)CC1] |
| 114 | 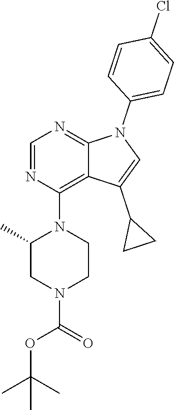 [SMILES: C[C@H]1CN(C(=O)OC(C)(C)C)CCN1c1ncnc2c1c(C1CC1)cn2-c1ccc(Cl)cc1] |
| 115 | 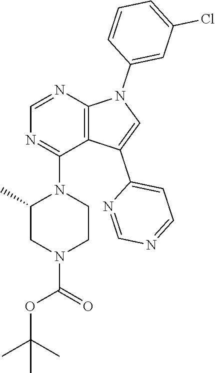 [SMILES: C[C@H]1CN(C(=O)OC(C)(C)C)CCN1c1ncnc2c1c(-c1ccncn1)cn2-c1cccc(Cl)c1] |
| 116 | 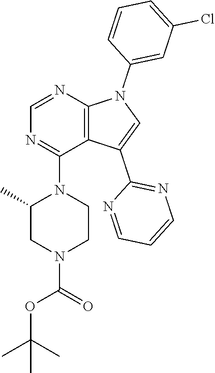 [SMILES: C[C@H]1CN(C(=O)OC(C)(C)C)CCN1c1ncnc2c1c(-c1ncccn1)cn2-c1cccc(Cl)c1] |
| 117 | 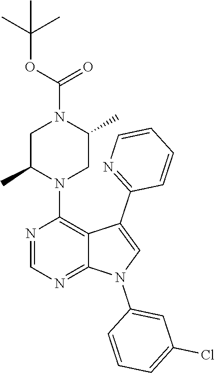 [SMILES: C[C@@H]1CN(c2ncnc3c2c(-c2ccccn2)cn3-c2cccc(Cl)c2)[C@@H](C)CN1C(=O)OC(C)(C)C] |
| 118 | 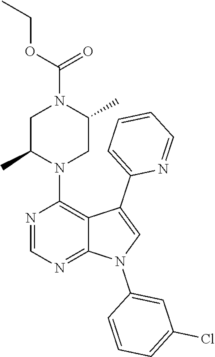 [SMILES: CCOC(=O)N1C[C@H](C)N(c2ncnc3c2c(-c2ccccn2)cn3-c2cccc(Cl)c2)C[C@H]1C] |
| 119 | 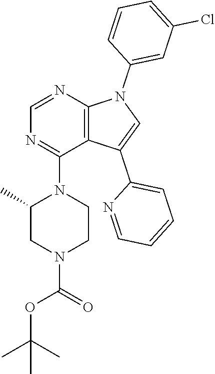 [SMILES: C[C@H]1CN(C(=O)OC(C)(C)C)CCN1c1ncnc2c1c(-c1ccccn1)cn2-c1cccc(Cl)c1] |
| 120 | 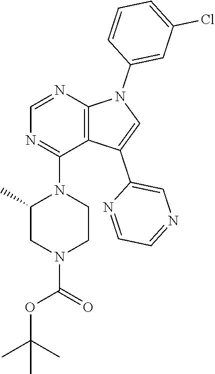 [SMILES: C[C@H]1CN(C(=O)OC(C)(C)C)CCN1c1ncnc2c1c(-c1cnccn1)cn2-c1cccc(Cl)c1] |
| 121 | 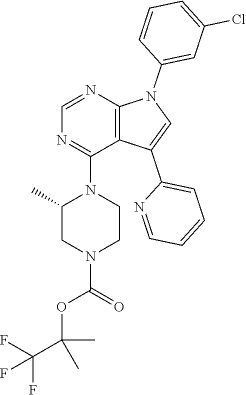 [SMILES: C[C@H]1CN(C(=O)OC(C)(C)C(F)(F)F)CCN1c1ncnc2c1c(-c1ccccn1)cn2-c1cccc(Cl)c1] |
| 122 | 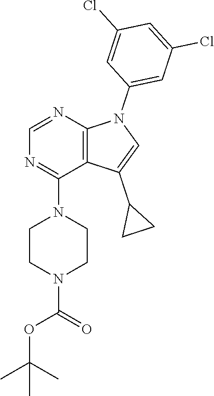 [SMILES: CC(C)(C)OC(=O)N1CCN(c2ncnc3c2c(C2CC2)cn3-c2cc(Cl)cc(Cl)c2)CC1] |
| 123 | 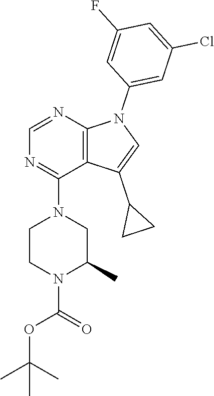 [SMILES: C[C@@H]1CN(c2ncnc3c2c(C2CC2)cn3-c2cc(F)cc(Cl)c2)CCN1C(=O)OC(C)(C)C] |
| 124 | 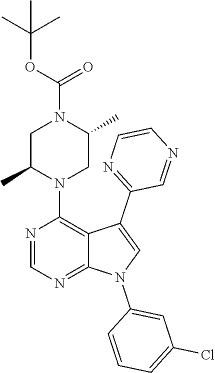 [SMILES: C[C@@H]1CN(c2ncnc3c2c(-c2cnccn2)cn3-c2cccc(Cl)c2)[C@@H](C)CN1C(=O)OC(C)(C)C] |
| 125 | 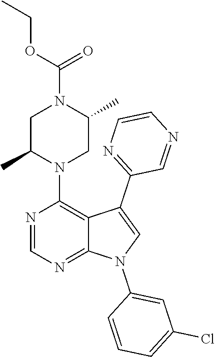 [SMILES: CCOC(=O)N1C[C@H](C)N(c2ncnc3c2c(-c2cnccn2)cn3-c2cccc(Cl)c2)C[C@H]1C] |
| 126 | 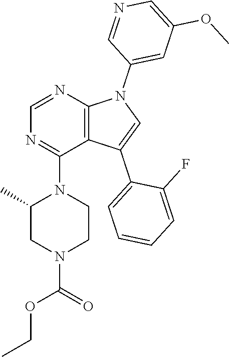 [SMILES: CCOC(=O)N1CCN(c2ncnc3c2c(-c2ccccc2F)cn3-c2cncc(OC)c2)[C@@H](C)C1] |
| 127 | 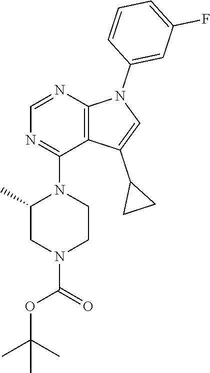 [SMILES: C[C@H]1CN(C(=O)OC(C)(C)C)CCN1c1ncnc2c1c(C1CC1)cn2-c1cccc(F)c1] |
| 128 | 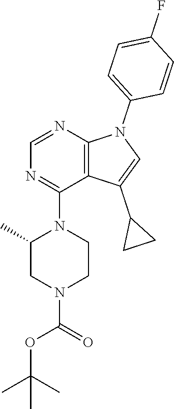 [SMILES: C[C@H]1CN(C(=O)OC(C)(C)C)CCN1c1ncnc2c1c(C1CC1)cn2-c1ccc(F)cc1] |
| 129 | 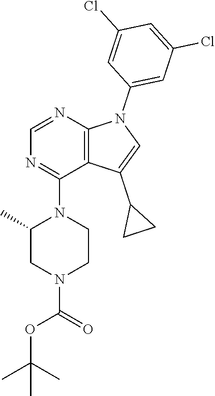 [SMILES: C[C@H]1CN(C(=O)OC(C)(C)C)CCN1c1ncnc2c1c(C1CC1)cn2-c1cc(Cl)cc(Cl)c1] |
| 130 | 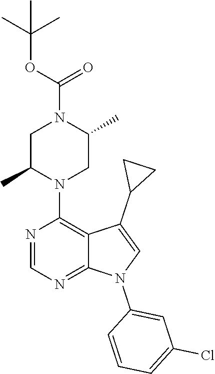 [SMILES: C[C@@H]1CN(c2ncnc3c2c(C2CC2)cn3-c2cccc(Cl)c2)[C@@H](C)CN1C(=O)OC(C)(C)C] |
| 131 | 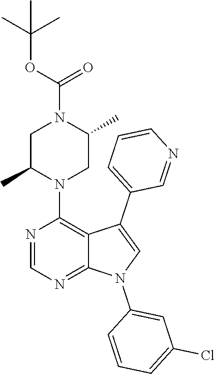 [SMILES: C[C@@H]1CN(c2ncnc3c2c(-c2cccnc2)cn3-c2cccc(Cl)c2)[C@@H](C)CN1C(=O)OC(C)(C)C] |
| 132 | 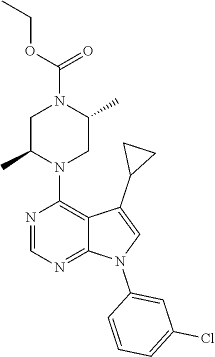 [SMILES: CCOC(=O)N1C[C@H](C)N(c2ncnc3c2c(C2CC2)cn3-c2cccc(Cl)c2)C[C@H]1C] |
| 133 | 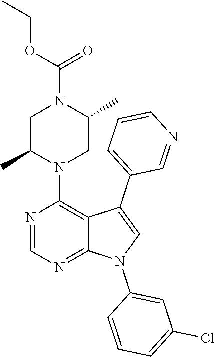 [SMILES: CCOC(=O)N1C[C@H](C)N(c2ncnc3c2c(-c2cccnc2)cn3-c2cccc(Cl)c2)C[C@H]1C] |
| 134 | 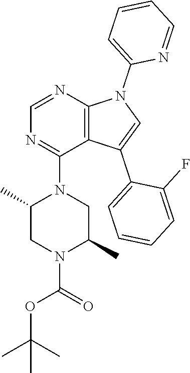 [SMILES: C[C@@H]1CN(c2ncnc3c2c(-c2ccccc2F)cn3-c2ccccn2)[C@@H](C)CN1C(=O)OC(C)(C)C] |
| 135 | 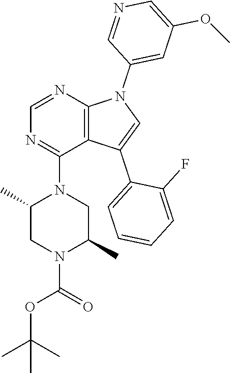 [SMILES: COc1cncc(-n2cc(-c3ccccc3F)c3c(N4C[C@@H](C)N(C(=O)OC(C)(C)C)C[C@@H]4C)ncnc32)c1] |
| 136 | 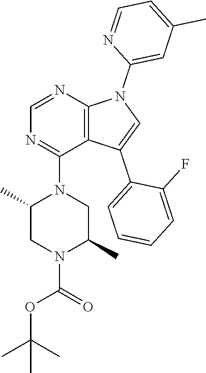 [SMILES: Cc1ccnc(-n2cc(-c3ccccc3F)c3c(N4C[C@@H](C)N(C(=O)OC(C)(C)C)C[C@@H]4C)ncnc32)c1] |
| 137 | 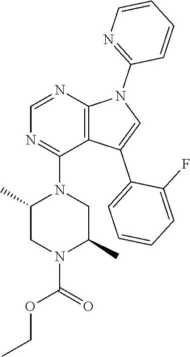 [SMILES: CCOC(=O)N1C[C@H](C)N(c2ncnc3c2c(-c2ccccc2F)cn3-c2ccccn2)C[C@H]1C] |
| 138 | 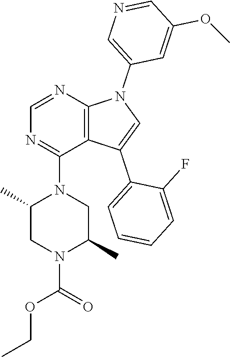 [SMILES: CCOC(=O)N1C[C@H](C)N(c2ncnc3c2c(-c2ccccc2F)cn3-c2cncc(OC)c2)C[C@H]1C] |
| 139 | 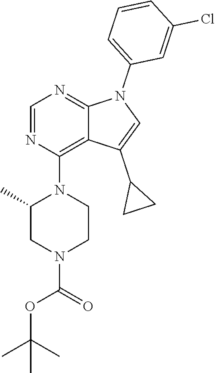 [SMILES: C[C@H]1CN(C(=O)OC(C)(C)C)CCN1c1ncnc2c1c(C1CC1)cn2-c1cccc(Cl)c1] |
| 140 | 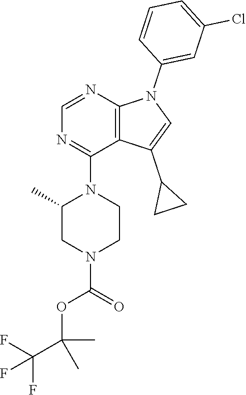 [SMILES: C[C@H]1CN(C(=O)OC(C)(C)C(F)(F)F)CCN1c1ncnc2c1c(C1CC1)cn2-c1cccc(Cl)c1] |
| 141 | 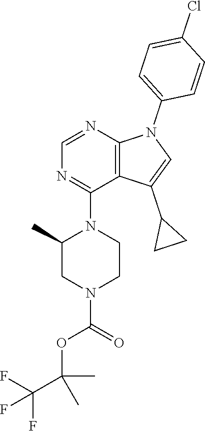 [SMILES: C[C@@H]1CN(C(=O)OC(C)(C)C(F)(F)F)CCN1c1ncnc2c1c(C1CC1)cn2-c1ccc(Cl)cc1] |
| 142 | 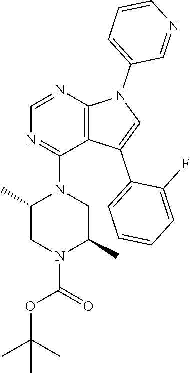 [SMILES: C[C@@H]1CN(c2ncnc3c2c(-c2ccccc2F)cn3-c2cccnc2)[C@@H](C)CN1C(=O)OC(C)(C)C] |
| 143 | 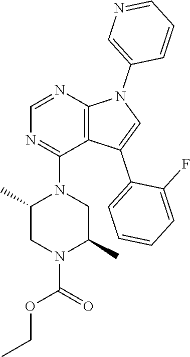 [SMILES: CCOC(=O)N1C[C@H](C)N(c2ncnc3c2c(-c2ccccc2F)cn3-c2cccnc2)C[C@H]1C] |
| 144 | 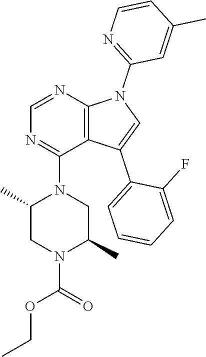 [SMILES: CCOC(=O)N1C[C@H](C)N(c2ncnc3c2c(-c2ccccc2F)cn3-c2cc(C)ccn2)C[C@H]1C] |
| 145 | 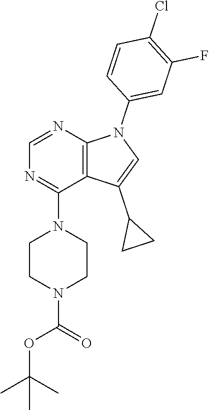 [SMILES: CC(C)(C)OC(=O)N1CCN(c2ncnc3c2c(C2CC2)cn3-c2ccc(Cl)c(F)c2)CC1] |
| 146 | 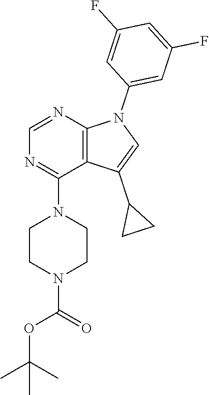 [SMILES: CC(C)(C)OC(=O)N1CCN(c2ncnc3c2c(C2CC2)cn3-c2cc(F)cc(F)c2)CC1] |
| 147 | 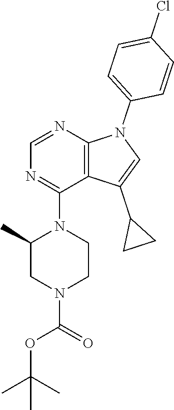 [SMILES: C[C@@H]1CN(C(=O)OC(C)(C)C)CCN1c1ncnc2c1c(C1CC1)cn2-c1ccc(Cl)cc1] |
| 148 | 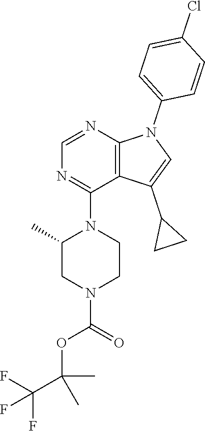 [SMILES: C[C@H]1CN(C(=O)OC(C)(C)C(F)(F)F)CCN1c1ncnc2c1c(C1CC1)cn2-c1ccc(Cl)cc1] |
| 149 | 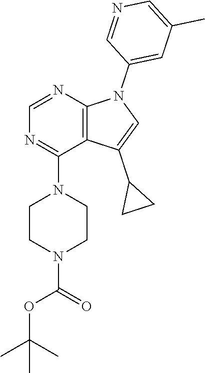 [SMILES: Cc1cncc(-n2cc(C3CC3)c3c(N4CCN(C(=O)OC(C)(C)C)CC4)ncnc32)c1] |
| 150 | 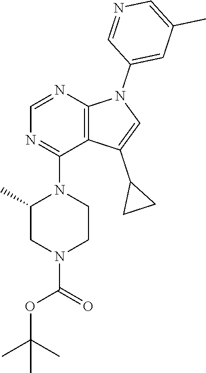 [SMILES: Cc1cncc(-n2cc(C3CC3)c3c(N4CCN(C(=O)OC(C)(C)C)C[C@@H]4C)ncnc32)c1] |
| 151 | 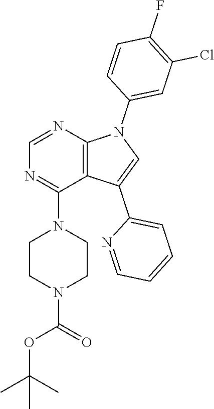 [SMILES: CC(C)(C)OC(=O)N1CCN(c2ncnc3c2c(-c2ccccn2)cn3-c2ccc(F)c(Cl)c2)CC1] |
| 152 | 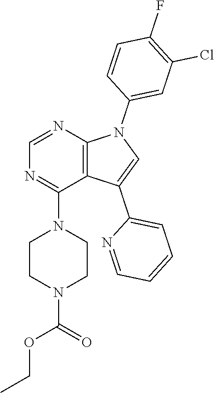 [SMILES: CCOC(=O)N1CCN(c2ncnc3c2c(-c2ccccn2)cn3-c2ccc(F)c(Cl)c2)CC1] |
| 153 | 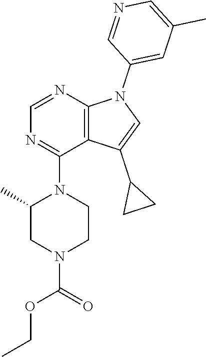 [SMILES: CCOC(=O)N1CCN(c2ncnc3c2c(C2CC2)cn3-c2cncc(C)c2)[C@@H](C)C1] |
| 154 | 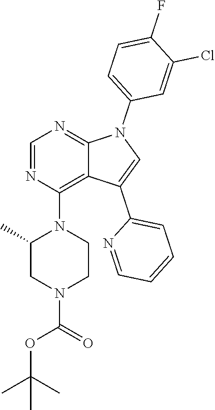 [SMILES: C[C@H]1CN(C(=O)OC(C)(C)C)CCN1c1ncnc2c1c(-c1ccccn1)cn2-c1ccc(F)c(Cl)c1] |
| 155 | 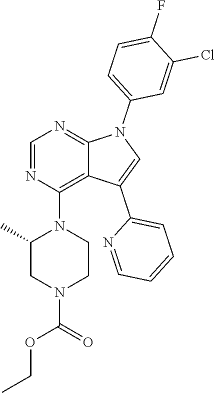 [SMILES: CCOC(=O)N1CCN(c2ncnc3c2c(-c2ccccn2)cn3-c2ccc(F)c(Cl)c2)[C@@H](C)C1] |
| 156 | 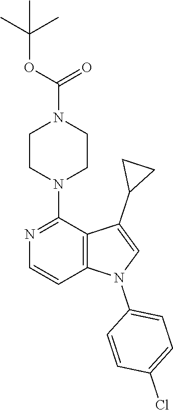 [SMILES: CC(C)(C)OC(=O)N1CCN(c2nccc3c2c(C2CC2)cn3-c2ccc(Cl)cc2)CC1] |
| 157 | 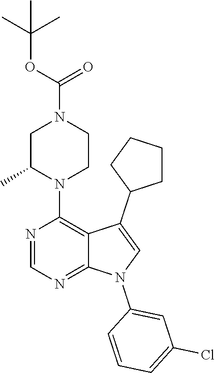 [SMILES: C[C@@H]1CN(C(=O)OC(C)(C)C)CCN1c1ncnc2c1c(C1CCCC1)cn2-c1cccc(Cl)c1] |
| 158 | 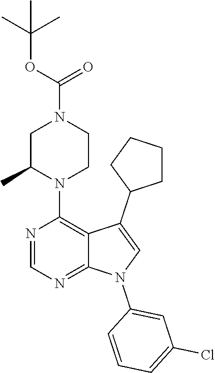 [SMILES: C[C@H]1CN(C(=O)OC(C)(C)C)CCN1c1ncnc2c1c(C1CCCC1)cn2-c1cccc(Cl)c1] |
| 159 | 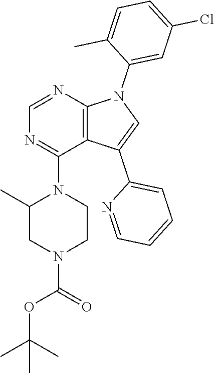 [SMILES: Cc1ccc(Cl)cc1-n1cc(-c2ccccn2)c2c(N3CCN(C(=O)OC(C)(C)C)CC3C)ncnc21] |
| 160 | 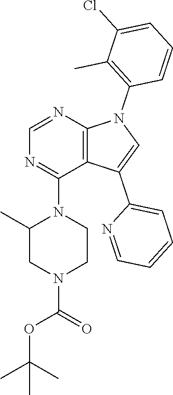 [SMILES: Cc1c(Cl)cccc1-n1cc(-c2ccccn2)c2c(N3CCN(C(=O)OC(C)(C)C)CC3C)ncnc21] |
| 161 | 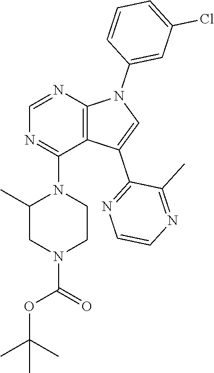 [SMILES: Cc1nccnc1-c1cn(-c2cccc(Cl)c2)c2ncnc(N3CCN(C(=O)OC(C)(C)C)CC3C)c12] |
| 162 | 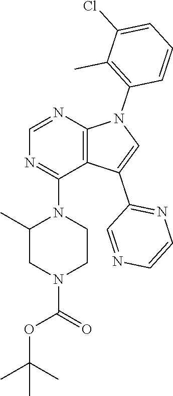 [SMILES: Cc1c(Cl)cccc1-n1cc(-c2cnccn2)c2c(N3CCN(C(=O)OC(C)(C)C)CC3C)ncnc21] |
| 163 | 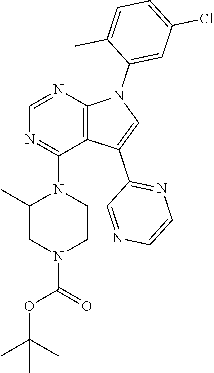 [SMILES: Cc1ccc(Cl)cc1-n1cc(-c2cnccn2)c2c(N3CCN(C(=O)OC(C)(C)C)CC3C)ncnc21] |
| 164 | 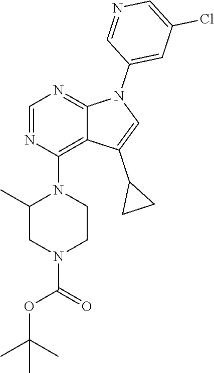 [SMILES: CC1CN(C(=O)OC(C)(C)C)CCN1c1ncnc2c1c(C1CC1)cn2-c1cncc(Cl)c1] |
| 165 | 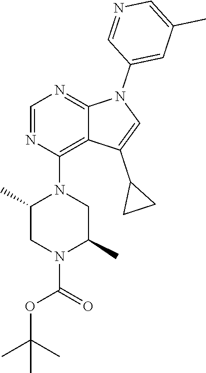 [SMILES: Cc1cncc(-n2cc(C3CC3)c3c(N4C[C@@H](C)N(C(=O)OC(C)(C)C)C[C@@H]4C)ncnc32)c1] |
| 166 | 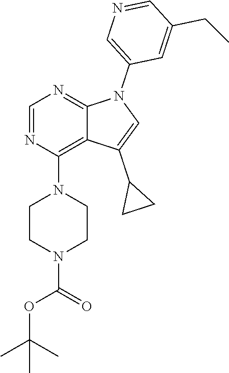 [SMILES: CCc1cncc(-n2cc(C3CC3)c3c(N4CCN(C(=O)OC(C)(C)C)CC4)ncnc32)c1] |
| 167 | 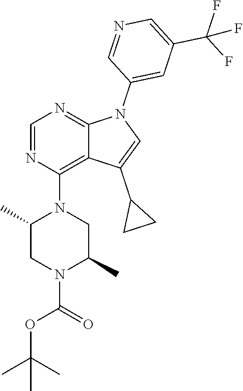 [SMILES: C[C@@H]1CN(c2ncnc3c2c(C2CC2)cn3-c2cncc(C(F)(F)F)c2)[C@@H](C)CN1C(=O)OC(C)(C)C] |
| 168 | 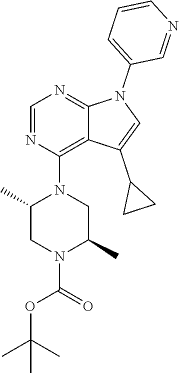 [SMILES: C[C@@H]1CN(c2ncnc3c2c(C2CC2)cn3-c2cccnc2)[C@@H](C)CN1C(=O)OC(C)(C)C] |
| 169 | 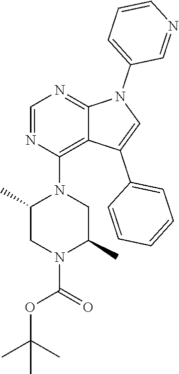 [SMILES: C[C@@H]1CN(c2ncnc3c2c(-c2ccccc2)cn3-c2cccnc2)[C@@H](C)CN1C(=O)OC(C)(C)C] |
| 170 | 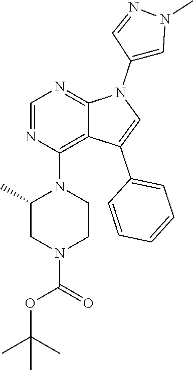 [SMILES: C[C@H]1CN(C(=O)OC(C)(C)C)CCN1c1ncnc2c1c(-c1ccccc1)cn2-c1cnn(C)c1] |
| 171 | 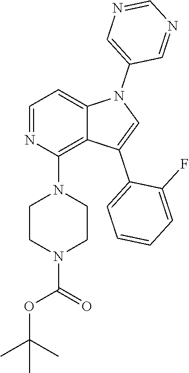 [SMILES: CC(C)(C)OC(=O)N1CCN(c2nccc3c2c(-c2ccccc2F)cn3-c2cncnc2)CC1] |
| 172 | 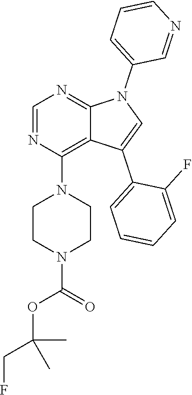 [SMILES: CC(C)(CF)OC(=O)N1CCN(c2ncnc3c2c(-c2ccccc2F)cn3-c2cccnc2)CC1] |
| 173 | 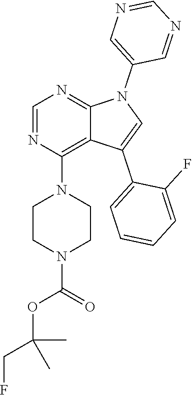 [SMILES: CC(C)(CF)OC(=O)N1CCN(c2ncnc3c2c(-c2ccccc2F)cn3-c2cncnc2)CC1] |
| 174 | 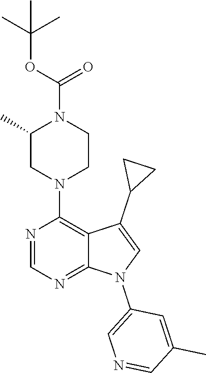 [SMILES: Cc1cncc(-n2cc(C3CC3)c3c(N4CCN(C(=O)OC(C)(C)C)[C@@H](C)C4)ncnc32)c1] |
| 175 | 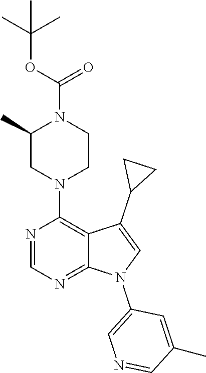 [SMILES: Cc1cncc(-n2cc(C3CC3)c3c(N4CCN(C(=O)OC(C)(C)C)[C@H](C)C4)ncnc32)c1] |
| 176 | 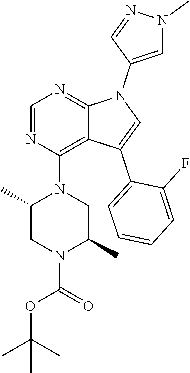 [SMILES: C[C@@H]1CN(c2ncnc3c2c(-c2ccccc2F)cn3-c2cnn(C)c2)[C@@H](C)CN1C(=O)OC(C)(C)C] |
| 177 | 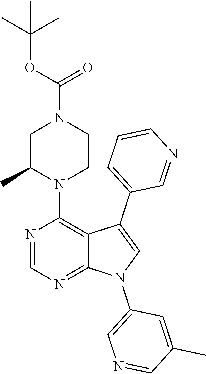 [SMILES: Cc1cncc(-n2cc(-c3cccnc3)c3c(N4CCN(C(=O)OC(C)(C)C)C[C@@H]4C)ncnc32)c1] |
| 178 | 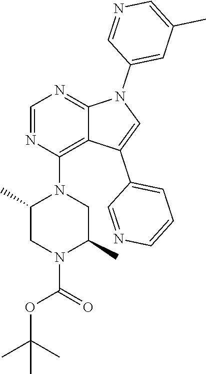 [SMILES: Cc1cncc(-n2cc(-c3cccnc3)c3c(N4C[C@@H](C)N(C(=O)OC(C)(C)C)C[C@@H]4C)ncnc32)c1] |
| 179 | 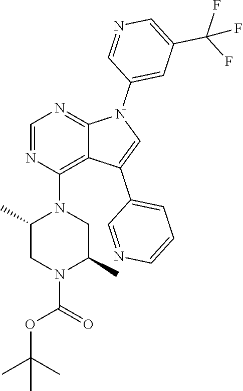 [SMILES: C[C@@H]1CN(c2ncnc3c2c(-c2cccnc2)cn3-c2cncc(C(F)(F)F)c2)[C@@H](C)CN1C(=O)OC(C)(C)C] |
| 180 | 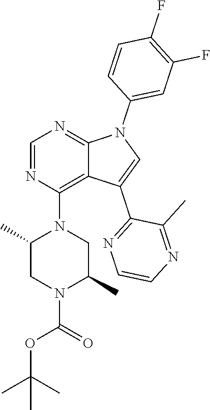 [SMILES: Cc1nccnc1-c1cn(-c2ccc(F)c(F)c2)c2ncnc(N3C[C@@H](C)N(C(=O)OC(C)(C)C)C[C@@H]3C)c12] |
| 181 | 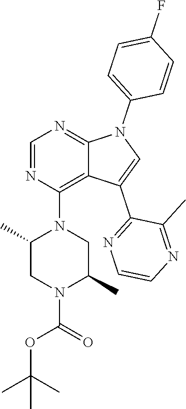 [SMILES: Cc1nccnc1-c1cn(-c2ccc(F)cc2)c2ncnc(N3C[C@@H](C)N(C(=O)OC(C)(C)C)C[C@@H]3C)c12] |
| 182 | 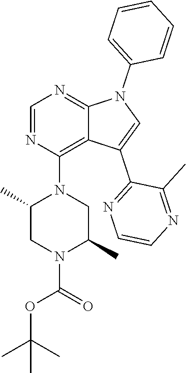 [SMILES: Cc1nccnc1-c1cn(-c2ccccc2)c2ncnc(N3C[C@@H](C)N(C(=O)OC(C)(C)C)C[C@@H]3C)c12] |
| 183 | 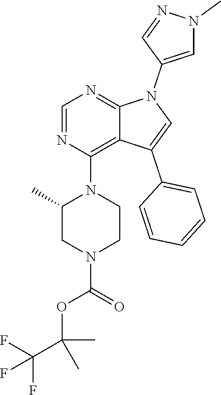 [SMILES: C[C@H]1CN(C(=O)OC(C)(C)C(F)(F)F)CCN1c1ncnc2c1c(-c1ccccc1)cn2-c1cnn(C)c1] |
| 184 | 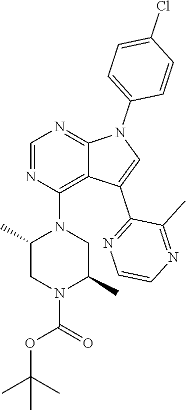 [SMILES: Cc1nccnc1-c1cn(-c2ccc(Cl)cc2)c2ncnc(N3C[C@@H](C)N(C(=O)OC(C)(C)C)C[C@@H]3C)c12] |
| 185 | 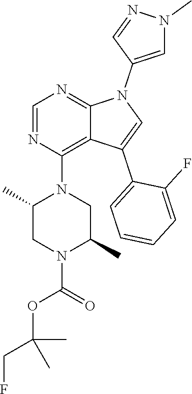 [SMILES: C[C@@H]1CN(c2ncnc3c2c(-c2ccccc2F)cn3-c2cnn(C)c2)[C@@H](C)CN1C(=O)OC(C)(C)CF] |
| 186 | 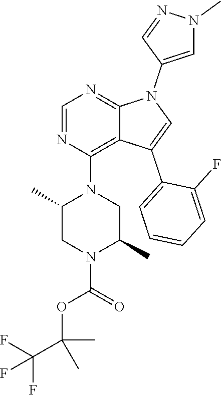 [SMILES: C[C@@H]1CN(c2ncnc3c2c(-c2ccccc2F)cn3-c2cnn(C)c2)[C@@H](C)CN1C(=O)OC(C)(C)C(F)(F)F] |
| 187 | 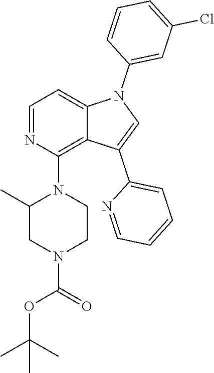 [SMILES: CC1CN(C(=O)OC(C)(C)C)CCN1c1nccc2c1c(-c1ccccn1)cn2-c1cccc(Cl)c1] |
| 188 | 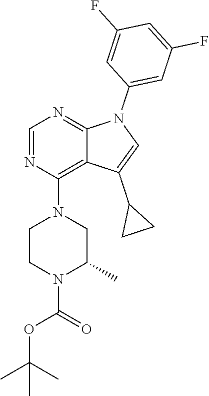 [SMILES: C[C@H]1CN(c2ncnc3c2c(C2CC2)cn3-c2cc(F)cc(F)c2)CCN1C(=O)OC(C)(C)C] |
| 189 | 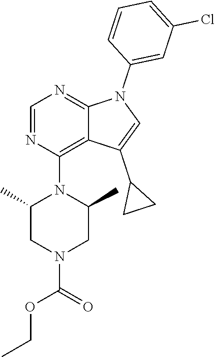 [SMILES: CCOC(=O)N1C[C@H](C)N(c2ncnc3c2c(C2CC2)cn3-c2cccc(Cl)c2)[C@@H](C)C1] |
| 190 | 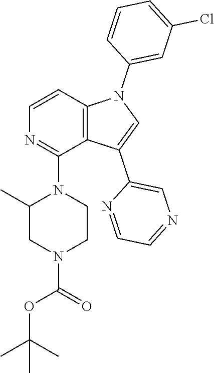 [SMILES: CC1CN(C(=O)OC(C)(C)C)CCN1c1nccc2c1c(-c1cnccn1)cn2-c1cccc(Cl)c1] |
| 191 | 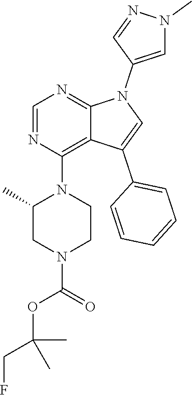 [SMILES: C[C@H]1CN(C(=O)OC(C)(C)CF)CCN1c1ncnc2c1c(-c1ccccc1)cn2-c1cnn(C)c1] |
| 192 | 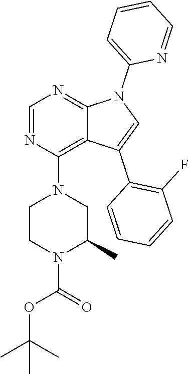 [SMILES: C[C@@H]1CN(c2ncnc3c2c(-c2ccccc2F)cn3-c2ccccn2)CCN1C(=O)OC(C)(C)C] |
| 193 | 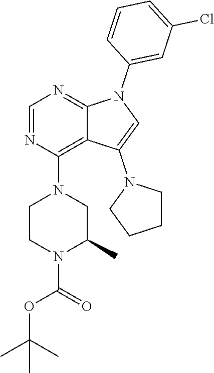 [SMILES: C[C@@H]1CN(c2ncnc3c2c(N2CCCC2)cn3-c2cccc(Cl)c2)CCN1C(=O)OC(C)(C)C] |
| 194 | 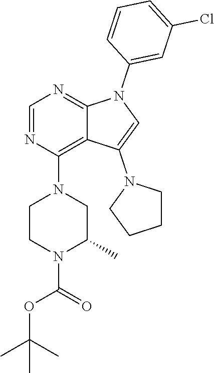 [SMILES: C[C@H]1CN(c2ncnc3c2c(N2CCCC2)cn3-c2cccc(Cl)c2)CCN1C(=O)OC(C)(C)C] |
| 195 | 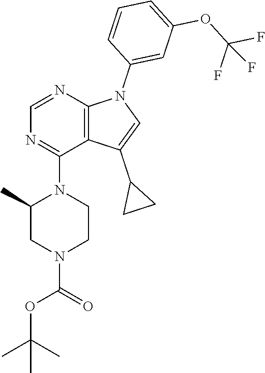 [SMILES: C[C@@H]1CN(C(=O)OC(C)(C)C)CCN1c1ncnc2c1c(C1CC1)cn2-c1cccc(OC(F)(F)F)c1] |
| 196 | 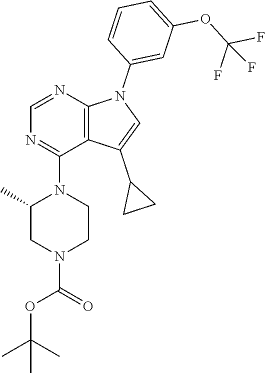 [SMILES: C[C@H]1CN(C(=O)OC(C)(C)C)CCN1c1ncnc2c1c(C1CC1)cn2-c1cccc(OC(F)(F)F)c1] |
| 197 | 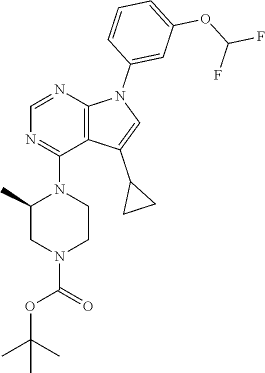 [SMILES: C[C@@H]1CN(C(=O)OC(C)(C)C)CCN1c1ncnc2c1c(C1CC1)cn2-c1cccc(OC(F)F)c1] |
| 198 | 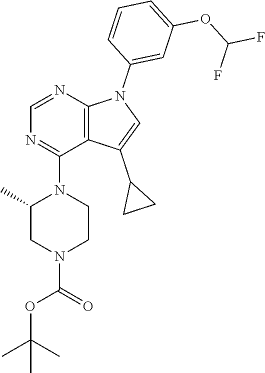 [SMILES: C[C@H]1CN(C(=O)OC(C)(C)C)CCN1c1ncnc2c1c(C1CC1)cn2-c1cccc(OC(F)F)c1] |
| 199 | 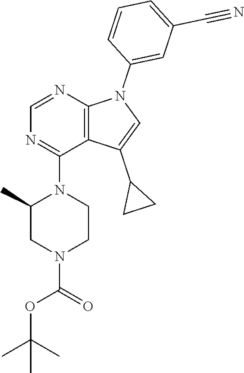 [SMILES: C[C@@H]1CN(C(=O)OC(C)(C)C)CCN1c1ncnc2c1c(C1CC1)cn2-c1cccc(C#N)c1] |
| 200 | 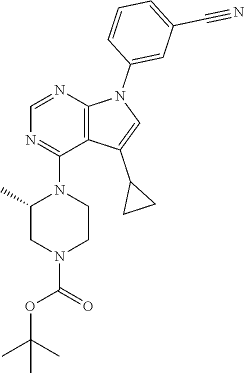 [SMILES: C[C@H]1CN(C(=O)OC(C)(C)C)CCN1c1ncnc2c1c(C1CC1)cn2-c1cccc(C#N)c1] |
| 201 | 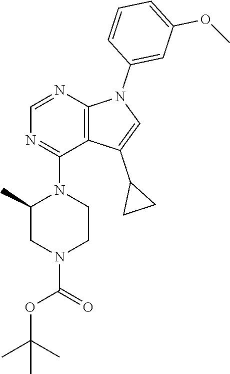 [SMILES: COc1cccc(-n2cc(C3CC3)c3c(N4CCN(C(=O)OC(C)(C)C)C[C@H]4C)ncnc32)c1] |
| 202 | 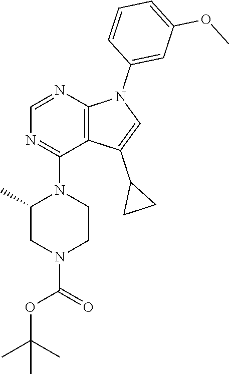 [SMILES: COc1cccc(-n2cc(C3CC3)c3c(N4CCN(C(=O)OC(C)(C)C)C[C@@H]4C)ncnc32)c1] |
| 203 | 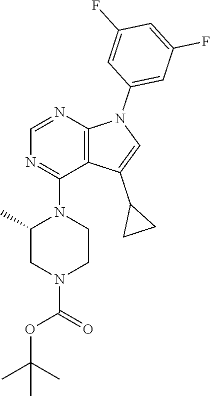 [SMILES: C[C@H]1CN(C(=O)OC(C)(C)C)CCN1c1ncnc2c1c(C1CC1)cn2-c1cc(F)cc(F)c1] |
| 204 | 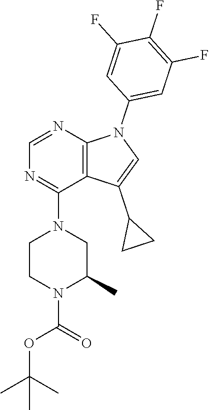 [SMILES: C[C@@H]1CN(c2ncnc3c2c(C2CC2)cn3-c2cc(F)c(F)c(F)c2)CCN1C(=O)OC(C)(C)C] |
| 205 | 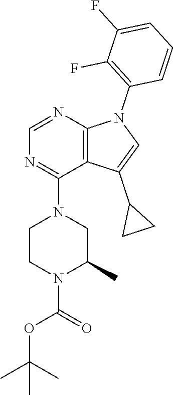 [SMILES: C[C@@H]1CN(c2ncnc3c2c(C2CC2)cn3-c2cccc(F)c2F)CCN1C(=O)OC(C)(C)C] |
| 206 | 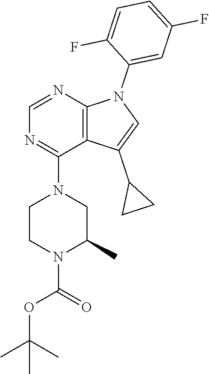 [SMILES: C[C@@H]1CN(c2ncnc3c2c(C2CC2)cn3-c2cc(F)ccc2F)CCN1C(=O)OC(C)(C)C] |
| 207 | 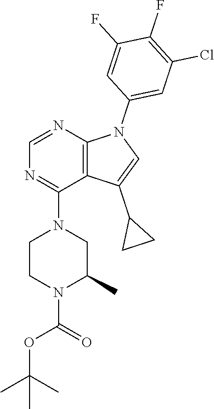 [SMILES: C[C@@H]1CN(c2ncnc3c2c(C2CC2)cn3-c2cc(F)c(F)c(Cl)c2)CCN1C(=O)OC(C)(C)C] |
| 208 | 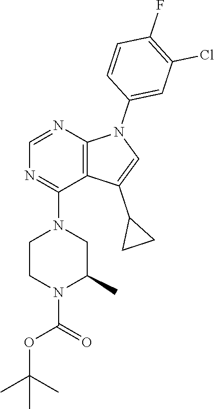 [SMILES: C[C@@H]1CN(c2ncnc3c2c(C2CC2)cn3-c2ccc(F)c(Cl)c2)CCN1C(=O)OC(C)(C)C] |
| 209 | 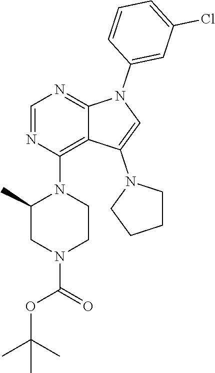 [SMILES: C[C@@H]1CN(C(=O)OC(C)(C)C)CCN1c1ncnc2c1c(N1CCCC1)cn2-c1cccc(Cl)c1] |
| 210 | 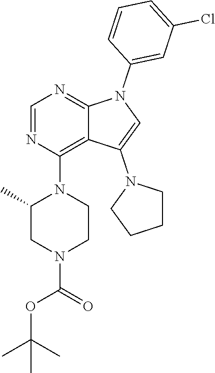 [SMILES: C[C@H]1CN(C(=O)OC(C)(C)C)CCN1c1ncnc2c1c(N1CCCC1)cn2-c1cccc(Cl)c1] |
| 211 | 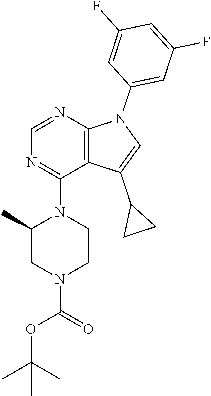 [SMILES: C[C@@H]1CN(C(=O)OC(C)(C)C)CCN1c1ncnc2c1c(C1CC1)cn2-c1cc(F)cc(F)c1] |
| 212 | 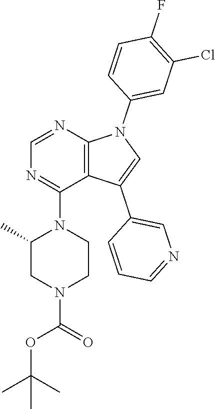 [SMILES: C[C@H]1CN(C(=O)OC(C)(C)C)CCN1c1ncnc2c1c(-c1cccnc1)cn2-c1ccc(F)c(Cl)c1] |
| 213 | 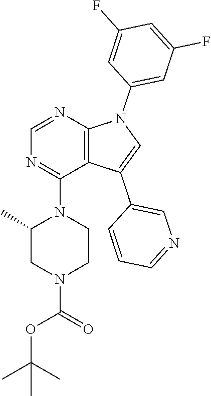 [SMILES: C[C@H]1CN(C(=O)OC(C)(C)C)CCN1c1ncnc2c1c(-c1cccnc1)cn2-c1cc(F)cc(F)c1] |
| 214 | 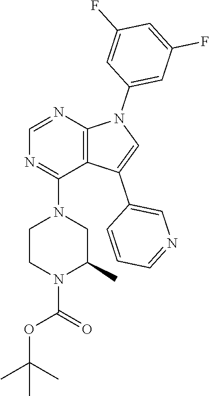 [SMILES: C[C@@H]1CN(c2ncnc3c2c(-c2cccnc2)cn3-c2cc(F)cc(F)c2)CCN1C(=O)OC(C)(C)C] |
| 215 | 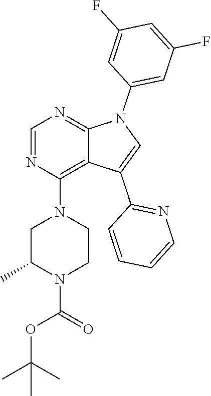 [SMILES: C[C@@H]1CN(c2ncnc3c2c(-c2ccccn2)cn3-c2cc(F)cc(F)c2)CCN1C(=O)OC(C)(C)C] |
| 216 | 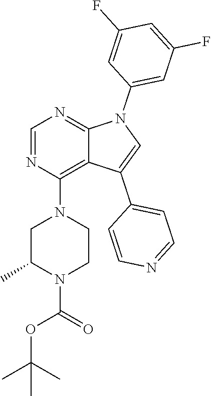 [SMILES: C[C@@H]1CN(c2ncnc3c2c(-c2ccncc2)cn3-c2cc(F)cc(F)c2)CCN1C(=O)OC(C)(C)C] |
| 217 | 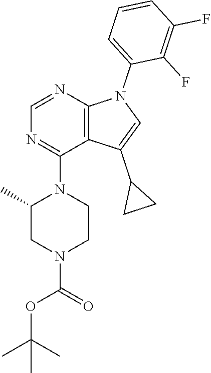 [SMILES: C[C@H]1CN(C(=O)OC(C)(C)C)CCN1c1ncnc2c1c(C1CC1)cn2-c1cccc(F)c1F] |
| 218 | 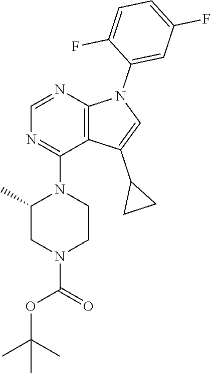 [SMILES: C[C@H]1CN(C(=O)OC(C)(C)C)CCN1c1ncnc2c1c(C1CC1)cn2-c1cc(F)ccc1F] |
| 219 | 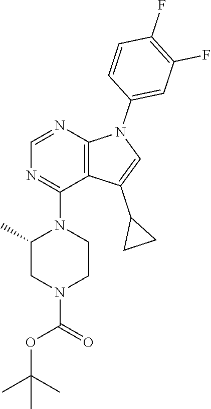 [SMILES: C[C@H]1CN(C(=O)OC(C)(C)C)CCN1c1ncnc2c1c(C1CC1)cn2-c1ccc(F)c(F)c1] |
| 220 | 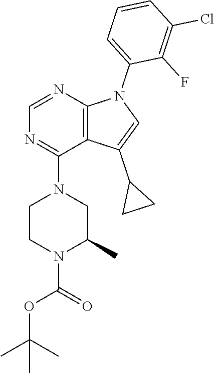 [SMILES: C[C@@H]1CN(c2ncnc3c2c(C2CC2)cn3-c2cccc(Cl)c2F)CCN1C(=O)OC(C)(C)C] |
| 221 | 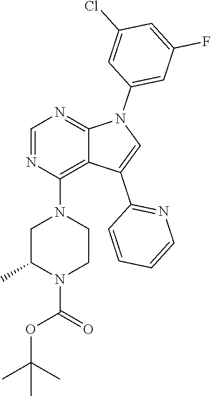 [SMILES: C[C@@H]1CN(c2ncnc3c2c(-c2ccccn2)cn3-c2cc(F)cc(Cl)c2)CCN1C(=O)OC(C)(C)C] |
| 222 | 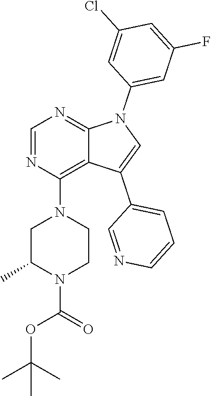 [SMILES: C[C@@H]1CN(c2ncnc3c2c(-c2cccnc2)cn3-c2cc(F)cc(Cl)c2)CCN1C(=O)OC(C)(C)C] |
| 223 | 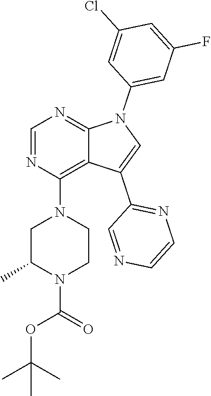 [SMILES: C[C@@H]1CN(c2ncnc3c2c(-c2cnccn2)cn3-c2cc(F)cc(Cl)c2)CCN1C(=O)OC(C)(C)C] |
| 224 | 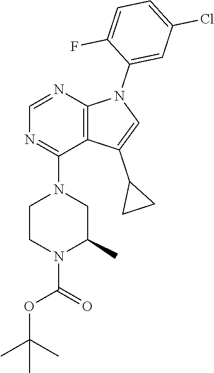 [SMILES: C[C@@H]1CN(c2ncnc3c2c(C2CC2)cn3-c2cc(Cl)ccc2F)CCN1C(=O)OC(C)(C)C] |
| 225 | 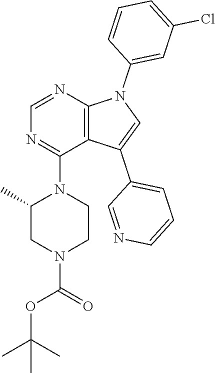 [SMILES: C[C@H]1CN(C(=O)OC(C)(C)C)CCN1c1ncnc2c1c(-c1cccnc1)cn2-c1cccc(Cl)c1] |
| 226 | 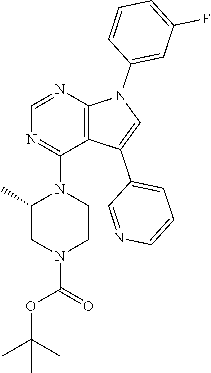 [SMILES: C[C@H]1CN(C(=O)OC(C)(C)C)CCN1c1ncnc2c1c(-c1cccnc1)cn2-c1cccc(F)c1] |
| 227 | 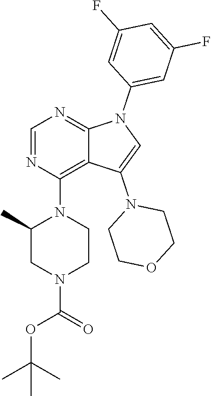 [SMILES: C[C@@H]1CN(C(=O)OC(C)(C)C)CCN1c1ncnc2c1c(N1CCOCC1)cn2-c1cc(F)cc(F)c1] |
| 228 | 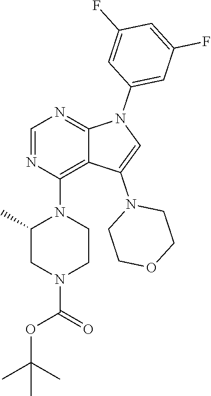 [SMILES: C[C@H]1CN(C(=O)OC(C)(C)C)CCN1c1ncnc2c1c(N1CCOCC1)cn2-c1cc(F)cc(F)c1] |
| 229 | 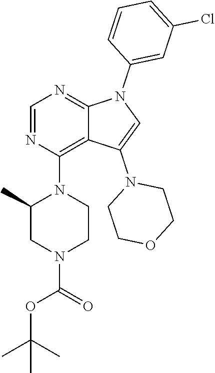 [SMILES: C[C@@H]1CN(C(=O)OC(C)(C)C)CCN1c1ncnc2c1c(N1CCOCC1)cn2-c1cccc(Cl)c1] |
| 230 | 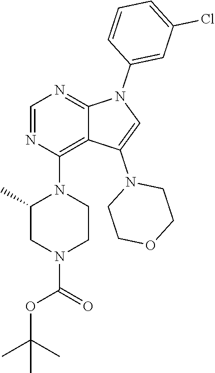 [SMILES: C[C@H]1CN(C(=O)OC(C)(C)C)CCN1c1ncnc2c1c(N1CCOCC1)cn2-c1cccc(Cl)c1] |
| 231 | 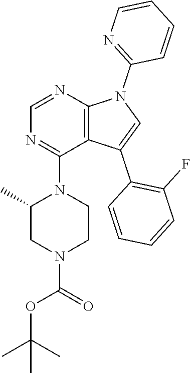 [SMILES: C[C@H]1CN(C(=O)OC(C)(C)C)CCN1c1ncnc2c1c(-c1ccccc1F)cn2-c1ccccn1] |
| 232 | 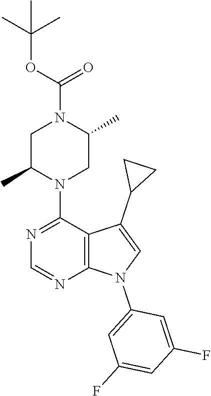 [SMILES: C[C@@H]1CN(c2ncnc3c2c(C2CC2)cn3-c2cc(F)cc(F)c2)[C@@H](C)CN1C(=O)OC(C)(C)C] |
| 233 | 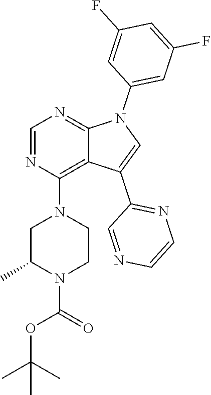 [SMILES: C[C@@H]1CN(c2ncnc3c2c(-c2cnccn2)cn3-c2cc(F)cc(F)c2)CCN1C(=O)OC(C)(C)C] |
| 234 | 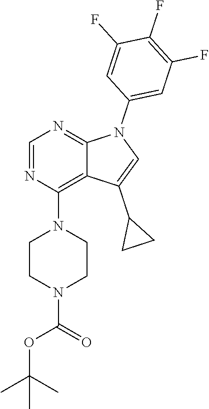 [SMILES: CC(C)(C)OC(=O)N1CCN(c2ncnc3c2c(C2CC2)cn3-c2cc(F)c(F)c(F)c2)CC1] |
| 235 | 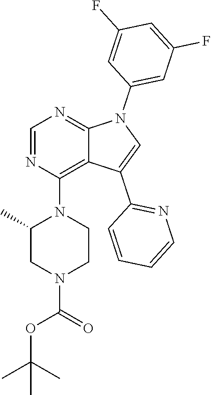 [SMILES: C[C@H]1CN(C(=O)OC(C)(C)C)CCN1c1ncnc2c1c(-c1ccccn1)cn2-c1cc(F)cc(F)c1] |
| 236 | 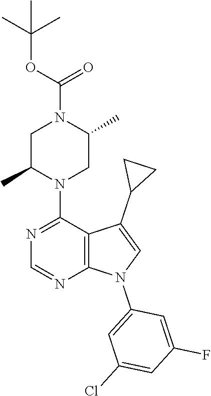 [SMILES: C[C@@H]1CN(c2ncnc3c2c(C2CC2)cn3-c2cc(F)cc(Cl)c2)[C@@H](C)CN1C(=O)OC(C)(C)C] |
| 237 | 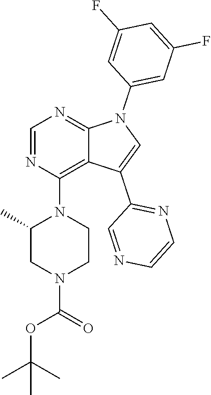 [SMILES: C[C@H]1CN(C(=O)OC(C)(C)C)CCN1c1ncnc2c1c(-c1cnccn1)cn2-c1cc(F)cc(F)c1] |
| 238 | 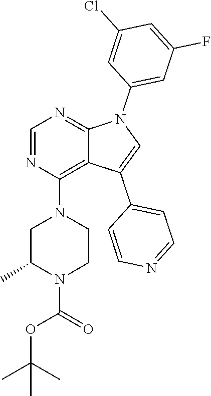 [SMILES: C[C@@H]1CN(c2ncnc3c2c(-c2ccncc2)cn3-c2cc(F)cc(Cl)c2)CCN1C(=O)OC(C)(C)C] |
| 239 | 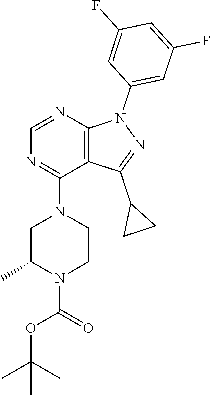 [SMILES: C[C@@H]1CN(c2ncnc3c2c(C2CC2)nn3-c2cc(F)cc(F)c2)CCN1C(=O)OC(C)(C)C] |
| 240 | 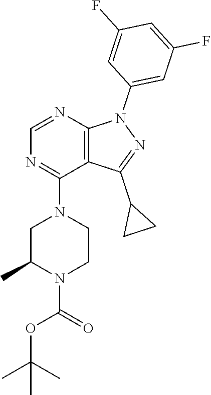 [SMILES: C[C@H]1CN(c2ncnc3c2c(C2CC2)nn3-c2cc(F)cc(F)c2)CCN1C(=O)OC(C)(C)C] |
| 241 | 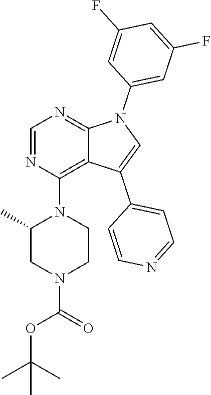 [SMILES: C[C@H]1CN(C(=O)OC(C)(C)C)CCN1c1ncnc2c1c(-c1ccncc1)cn2-c1cc(F)cc(F)c1] |
| 242 | 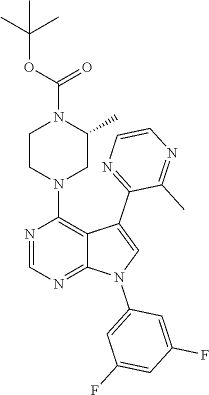 [SMILES: Cc1nccnc1-c1cn(-c2cc(F)cc(F)c2)c2ncnc(N3CCN(C(=O)OC(C)(C)C)[C@H](C)C3)c12] |
| 243 | 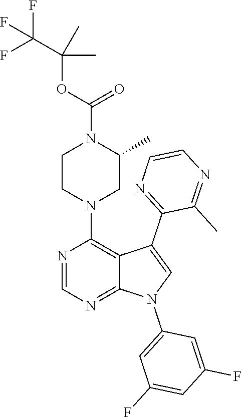 [SMILES: Cc1nccnc1-c1cn(-c2cc(F)cc(F)c2)c2ncnc(N3CCN(C(=O)OC(C)(C)C(F)(F)F)[C@H](C)C3)c12] |
| 244 | 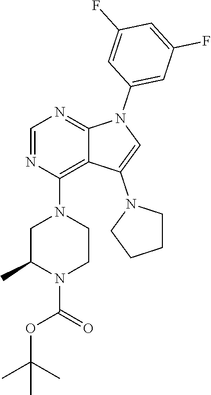 [SMILES: C[C@H]1CN(c2ncnc3c2c(N2CCCC2)cn3-c2cc(F)cc(F)c2)CCN1C(=O)OC(C)(C)C] |
| 245 | 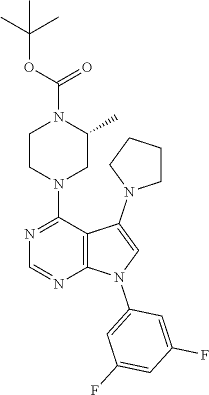 [SMILES: C[C@@H]1CN(c2ncnc3c2c(N2CCCC2)cn3-c2cc(F)cc(F)c2)CCN1C(=O)OC(C)(C)C] |
| 246 | 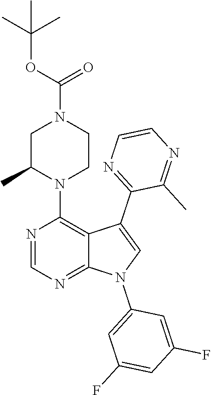 [SMILES: Cc1nccnc1-c1cn(-c2cc(F)cc(F)c2)c2ncnc(N3CCN(C(=O)OC(C)(C)C)C[C@@H]3C)c12] |
| 247 | 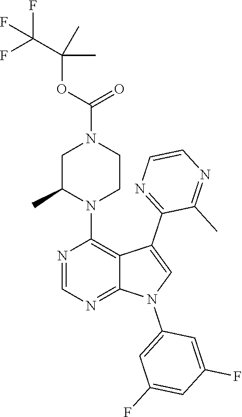 [SMILES: Cc1nccnc1-c1cn(-c2cc(F)cc(F)c2)c2ncnc(N3CCN(C(=O)OC(C)(C)C(F)(F)F)C[C@@H]3C)c12] |
| 248 | 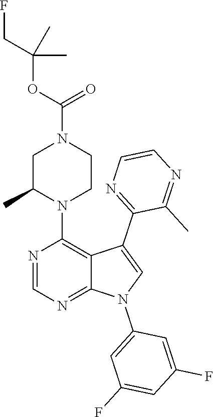 [SMILES: Cc1nccnc1-c1cn(-c2cc(F)cc(F)c2)c2ncnc(N3CCN(C(=O)OC(C)(C)CF)C[C@@H]3C)c12] |
| 249 | 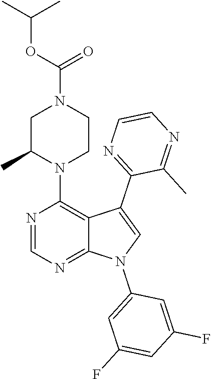 [SMILES: Cc1nccnc1-c1cn(-c2cc(F)cc(F)c2)c2ncnc(N3CCN(C(=O)OC(C)C)C[C@@H]3C)c12] |
| 250 | 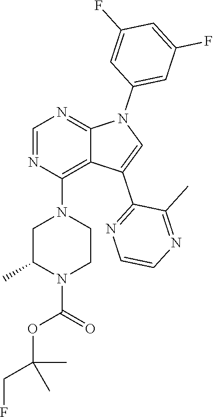 [SMILES: Cc1nccnc1-c1cn(-c2cc(F)cc(F)c2)c2ncnc(N3CCN(C(=O)OC(C)(C)CF)[C@H](C)C3)c12] |
| 251 | 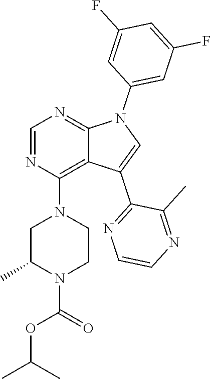 [SMILES: Cc1nccnc1-c1cn(-c2cc(F)cc(F)c2)c2ncnc(N3CCN(C(=O)OC(C)C)[C@H](C)C3)c12] |
| 252 | 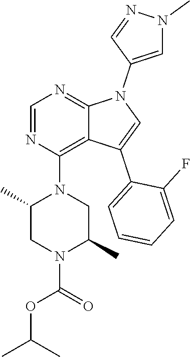 [SMILES: CC(C)OC(=O)N1C[C@H](C)N(c2ncnc3c2c(-c2ccccc2F)cn3-c2cnn(C)c2)C[C@H]1C] |
| 253 | 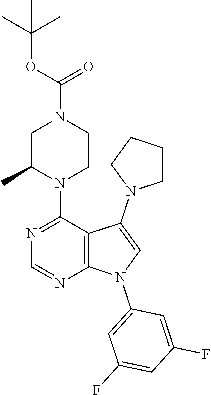 [SMILES: C[C@H]1CN(C(=O)OC(C)(C)C)CCN1c1ncnc2c1c(N1CCCC1)cn2-c1cc(F)cc(F)c1] |
| 254 | 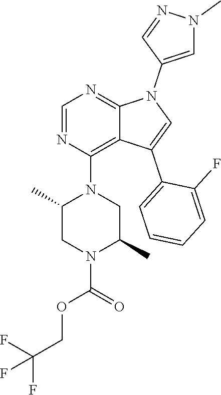 [SMILES: C[C@@H]1CN(c2ncnc3c2c(-c2ccccc2F)cn3-c2cnn(C)c2)[C@@H](C)CN1C(=O)OCC(F)(F)F] |
| 255 | 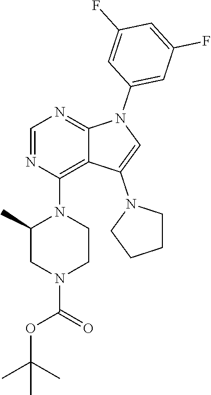 [SMILES: C[C@@H]1CN(C(=O)OC(C)(C)C)CCN1c1ncnc2c1c(N1CCCC1)cn2-c1cc(F)cc(F)c1] |
| 256 | 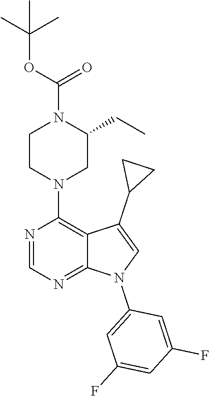 [SMILES: CC[C@@H]1CN(c2ncnc3c2c(C2CC2)cn3-c2cc(F)cc(F)c2)CCN1C(=O)OC(C)(C)C] |
| 257 | 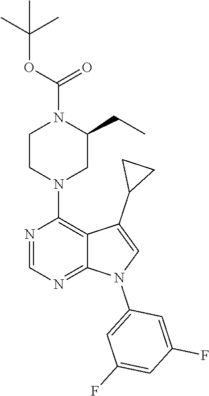 [SMILES: CC[C@H]1CN(c2ncnc3c2c(C2CC2)cn3-c2cc(F)cc(F)c2)CCN1C(=O)OC(C)(C)C] |
| 258 | 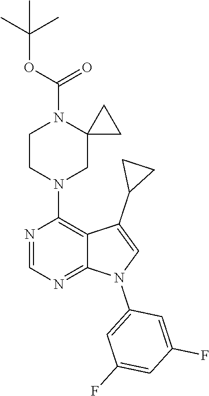 [SMILES: CC(C)(C)OC(=O)N1CCN(c2ncnc3c2c(C2CC2)cn3-c2cc(F)cc(F)c2)CC12CC2] |
| 259 | 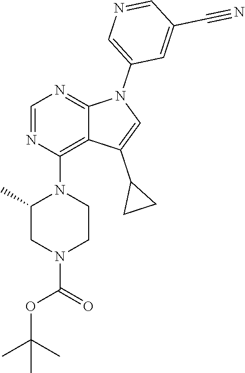 [SMILES: C[C@H]1CN(C(=O)OC(C)(C)C)CCN1c1ncnc2c1c(C1CC1)cn2-c1cncc(C#N)c1] |
| 260 | 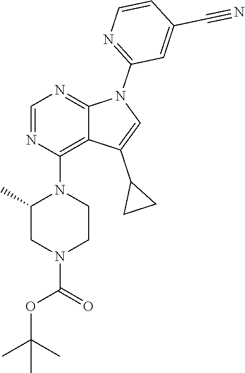 [SMILES: C[C@H]1CN(C(=O)OC(C)(C)C)CCN1c1ncnc2c1c(C1CC1)cn2-c1cc(C#N)ccn1] |
| 261 | 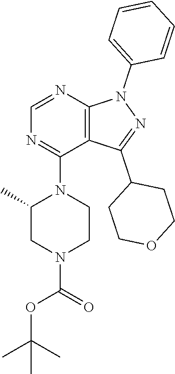 [SMILES: C[C@H]1CN(C(=O)OC(C)(C)C)CCN1c1ncnc2c1c(C1CCOCC1)nn2-c1ccccc1] |
| 262 | 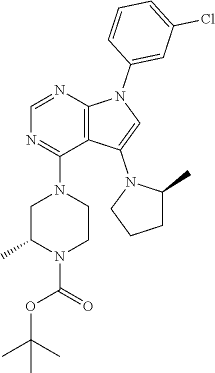 [SMILES: C[C@@H]1CN(c2ncnc3c2c(N2CCC[C@@H]2C)cn3-c2cccc(Cl)c2)CCN1C(=O)OC(C)(C)C] |
| 263 | 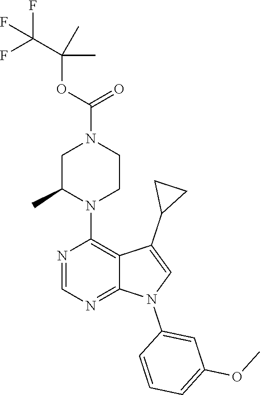 [SMILES: COc1cccc(-n2cc(C3CC3)c3c(N4CCN(C(=O)OC(C)(C)C(F)(F)F)C[C@@H]4C)ncnc32)c1] |
| 264 | 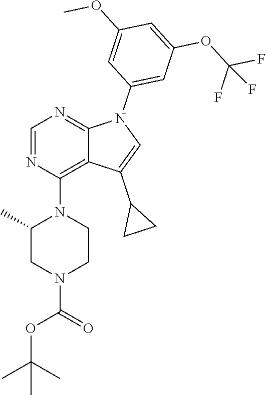 [SMILES: COc1cc(OC(F)(F)F)cc(-n2cc(C3CC3)c3c(N4CCN(C(=O)OC(C)(C)C)C[C@@H]4C)ncnc32)c1] |
| 265 | 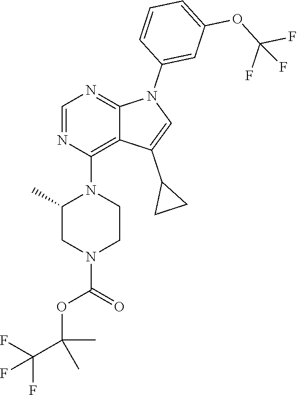 [SMILES: C[C@H]1CN(C(=O)OC(C)(C)C(F)(F)F)CCN1c1ncnc2c1c(C1CC1)cn2-c1cccc(OC(F)(F)F)c1] |
| 266 | 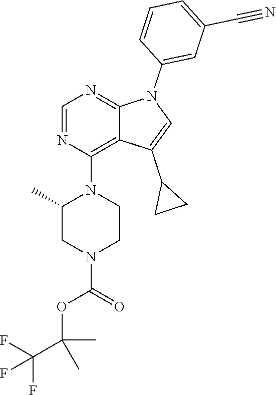 [SMILES: C[C@H]1CN(C(=O)OC(C)(C)C(F)(F)F)CCN1c1ncnc2c1c(C1CC1)cn2-c1cccc(C#N)c1] |
| 267 | 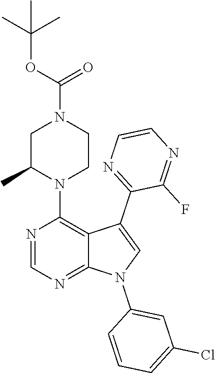 [SMILES: C[C@H]1CN(C(=O)OC(C)(C)C)CCN1c1ncnc2c1c(-c1nccnc1F)cn2-c1cccc(Cl)c1] |
| 268 | 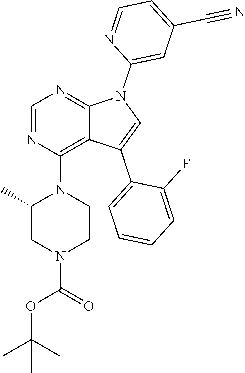 [SMILES: C[C@H]1CN(C(=O)OC(C)(C)C)CCN1c1ncnc2c1c(-c1ccccc1F)cn2-c1cc(C#N)ccn1] |
| 269 | 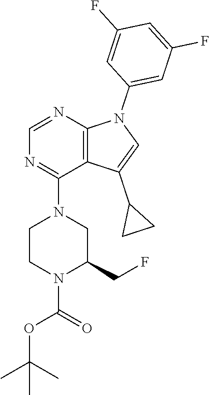 [SMILES: CC(C)(C)OC(=O)N1CCN(c2ncnc3c2c(C2CC2)cn3-c2cc(F)cc(F)c2)C[C@H]1CF] |
| 270 | 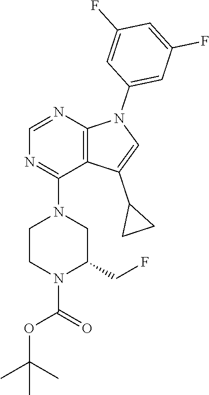 [SMILES: CC(C)(C)OC(=O)N1CCN(c2ncnc3c2c(C2CC2)cn3-c2cc(F)cc(F)c2)C[C@@H]1CF] |
| 271 | 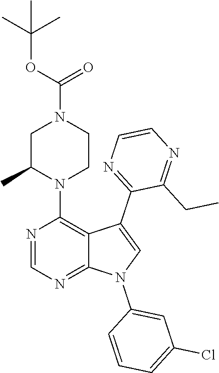 [SMILES: CCc1nccnc1-c1cn(-c2cccc(Cl)c2)c2ncnc(N3CCN(C(=O)OC(C)(C)C)C[C@@H]3C)c12] |
| 272 | 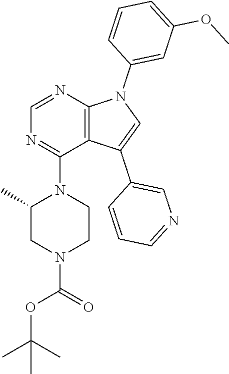 [SMILES: COc1cccc(-n2cc(-c3cccnc3)c3c(N4CCN(C(=O)OC(C)(C)C)C[C@@H]4C)ncnc32)c1] |
| 273 | 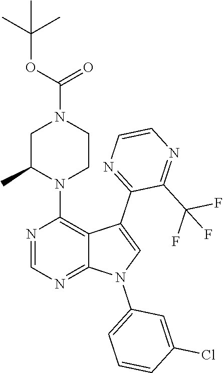 [SMILES: C[C@H]1CN(C(=O)OC(C)(C)C)CCN1c1ncnc2c1c(-c1nccnc1C(F)(F)F)cn2-c1cccc(Cl)c1] |
| 274 | 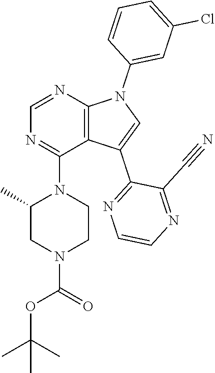 [SMILES: C[C@H]1CN(C(=O)OC(C)(C)C)CCN1c1ncnc2c1c(-c1nccnc1C#N)cn2-c1cccc(Cl)c1] |
| 275 | 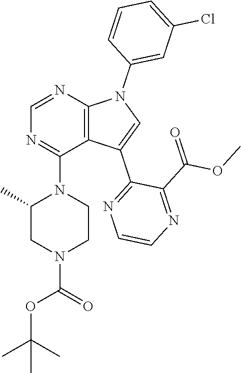 [SMILES: COC(=O)c1nccnc1-c1cn(-c2cccc(Cl)c2)c2ncnc(N3CCN(C(=O)OC(C)(C)C)C[C@@H]3C)c12] |
| 276 | 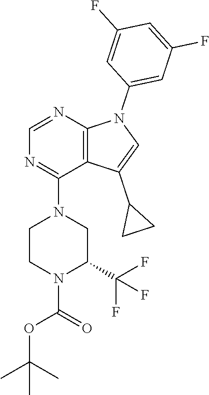 [SMILES: CC(C)(C)OC(=O)N1CCN(c2ncnc3c2c(C2CC2)cn3-c2cc(F)cc(F)c2)C[C@@H]1C(F)(F)F] |
| 277 | 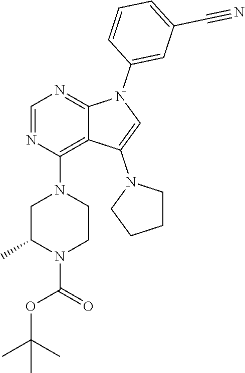 [SMILES: C[C@@H]1CN(c2ncnc3c2c(N2CCCC2)cn3-c2cccc(C#N)c2)CCN1C(=O)OC(C)(C)C] |
| 278 | 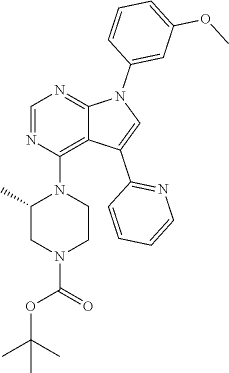 [SMILES: COc1cccc(-n2cc(-c3ccccn3)c3c(N4CCN(C(=O)OC(C)(C)C)C[C@@H]4C)ncnc32)c1] |
| 279 | 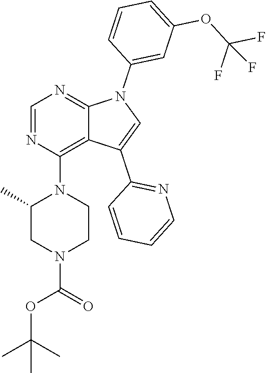 [SMILES: C[C@H]1CN(C(=O)OC(C)(C)C)CCN1c1ncnc2c1c(-c1ccccn1)cn2-c1cccc(OC(F)(F)F)c1] |
| 280 | 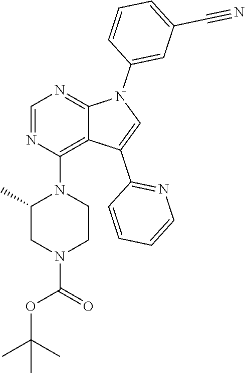 [SMILES: C[C@H]1CN(C(=O)OC(C)(C)C)CCN1c1ncnc2c1c(-c1ccccn1)cn2-c1cccc(C#N)c1] |
| 281 | 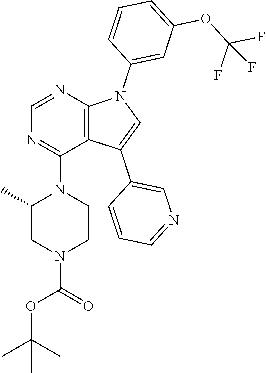 [SMILES: C[C@H]1CN(C(=O)OC(C)(C)C)CCN1c1ncnc2c1c(-c1cccnc1)cn2-c1cccc(OC(F)(F)F)c1] |
| 282 | 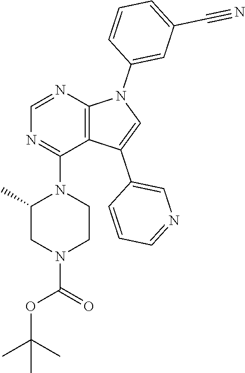 [SMILES: C[C@H]1CN(C(=O)OC(C)(C)C)CCN1c1ncnc2c1c(-c1cccnc1)cn2-c1cccc(C#N)c1] |
| 283 | 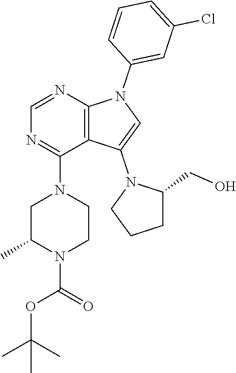 [SMILES: C[C@@H]1CN(c2ncnc3c2c(N2CCC[C@H]2CO)cn3-c2cccc(Cl)c2)CCN1C(=O)OC(C)(C)C] |
| 284 | 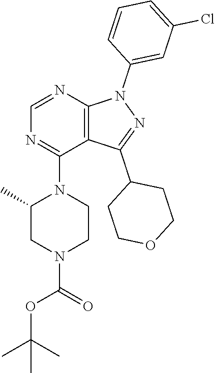 [SMILES: C[C@H]1CN(C(=O)OC(C)(C)C)CCN1c1ncnc2c1c(C1CCOCC1)nn2-c1cccc(Cl)c1] |
| 285 | 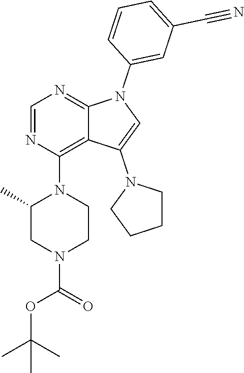 [SMILES: C[C@H]1CN(C(=O)OC(C)(C)C)CCN1c1ncnc2c1c(N1CCCC1)cn2-c1cccc(C#N)c1] |
| 286 | 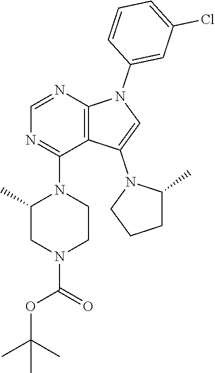 [SMILES: C[C@@H]1CCCN1c1cn(-c2cccc(Cl)c2)c2ncnc(N3CCN(C(=O)OC(C)(C)C)C[C@@H]3C)c12] |
| 287 | 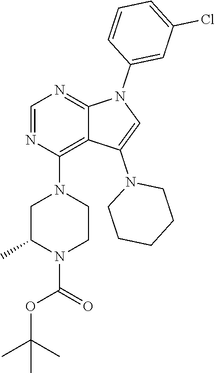 [SMILES: C[C@@H]1CN(c2ncnc3c2c(N2CCCCC2)cn3-c2cccc(Cl)c2)CCN1C(=O)OC(C)(C)C] |
| 288 | 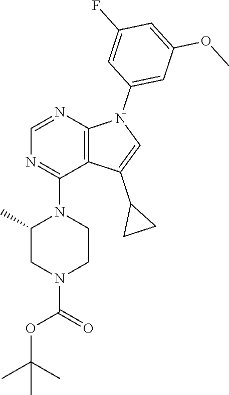 [SMILES: COc1cc(F)cc(-n2cc(C3CC3)c3c(N4CCN(C(=O)OC(C)(C)C)C[C@@H]4C)ncnc32)c1] |
| 289 | 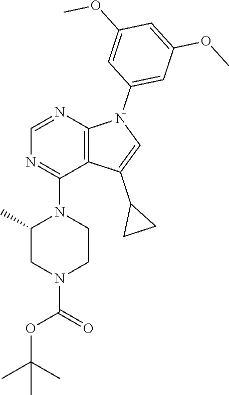 [SMILES: COc1cc(OC)cc(-n2cc(C3CC3)c3c(N4CCN(C(=O)OC(C)(C)C)C[C@@H]4C)ncnc32)c1] |
| 290 | 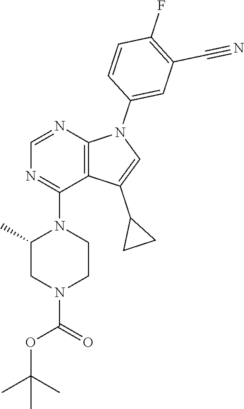 [SMILES: C[C@H]1CN(C(=O)OC(C)(C)C)CCN1c1ncnc2c1c(C1CC1)cn2-c1ccc(F)c(C#N)c1] |
| 291 | 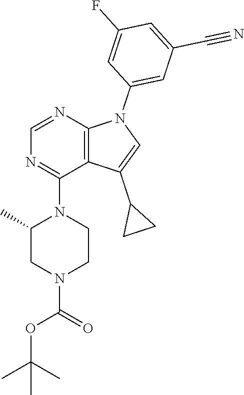 [SMILES: C[C@H]1CN(C(=O)OC(C)(C)C)CCN1c1ncnc2c1c(C1CC1)cn2-c1cc(F)cc(C#N)c1] |
| 292 | 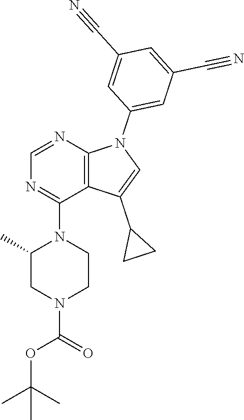 [SMILES: C[C@H]1CN(C(=O)OC(C)(C)C)CCN1c1ncnc2c1c(C1CC1)cn2-c1cc(C#N)cc(C#N)c1] |
| 293 | 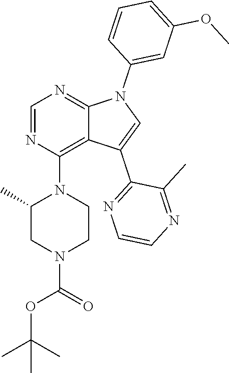 [SMILES: COc1cccc(-n2cc(-c3nccnc3C)c3c(N4CCN(C(=O)OC(C)(C)C)C[C@@H]4C)ncnc32)c1] |
| 294 | 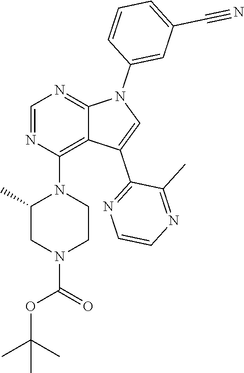 [SMILES: Cc1nccnc1-c1cn(-c2cccc(C#N)c2)c2ncnc(N3CCN(C(=O)OC(C)(C)C)C[C@@H]3C)c12] |
| 295 | 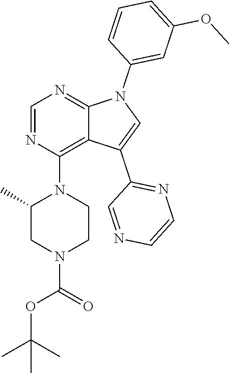 [SMILES: COc1cccc(-n2cc(-c3cnccn3)c3c(N4CCN(C(=O)OC(C)(C)C)C[C@@H]4C)ncnc32)c1] |
| 296 | 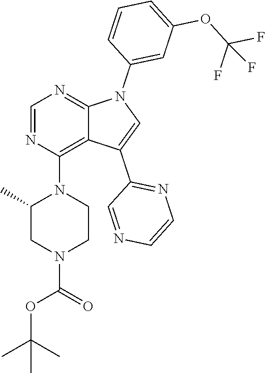 [SMILES: C[C@H]1CN(C(=O)OC(C)(C)C)CCN1c1ncnc2c1c(-c1cnccn1)cn2-c1cccc(OC(F)(F)F)c1] |
| 297 | 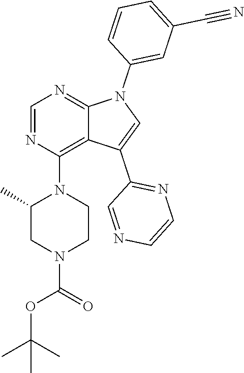 [SMILES: C[C@H]1CN(C(=O)OC(C)(C)C)CCN1c1ncnc2c1c(-c1cnccn1)cn2-c1cccc(C#N)c1] |
| 298 | 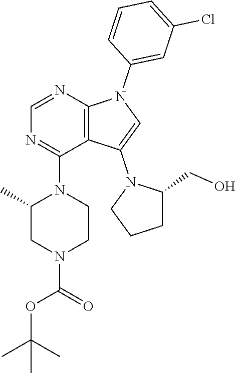 [SMILES: C[C@H]1CN(C(=O)OC(C)(C)C)CCN1c1ncnc2c1c(N1CCC[C@H]1CO)cn2-c1cccc(Cl)c1] |
| 299 | 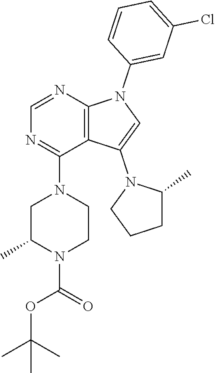 [SMILES: C[C@@H]1CN(c2ncnc3c2c(N2CCC[C@H]2C)cn3-c2cccc(Cl)c2)CCN1C(=O)OC(C)(C)C] |
| 300 | 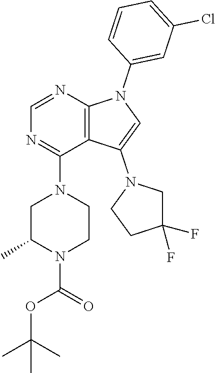 [SMILES: C[C@@H]1CN(c2ncnc3c2c(N2CCC(F)(F)C2)cn3-c2cccc(Cl)c2)CCN1C(=O)OC(C)(C)C] |
| 301 | 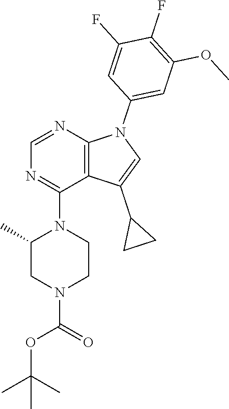 [SMILES: COc1cc(-n2cc(C3CC3)c3c(N4CCN(C(=O)OC(C)(C)C)C[C@@H]4C)ncnc32)cc(F)c1F] |
| 302 | 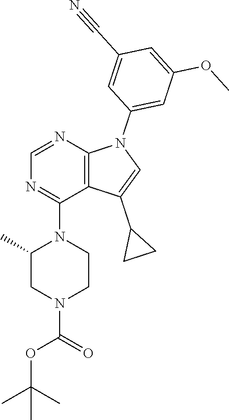 [SMILES: COc1cc(C#N)cc(-n2cc(C3CC3)c3c(N4CCN(C(=O)OC(C)(C)C)C[C@@H]4C)ncnc32)c1] |
| 303 | 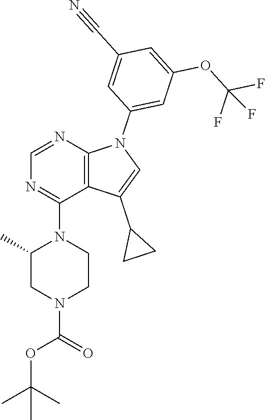 [SMILES: C[C@H]1CN(C(=O)OC(C)(C)C)CCN1c1ncnc2c1c(C1CC1)cn2-c1cc(C#N)cc(OC(F)(F)F)c1] |
| 304 | 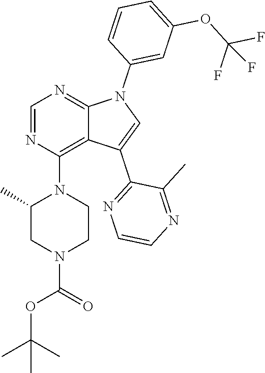 [SMILES: Cc1nccnc1-c1cn(-c2cccc(OC(F)(F)F)c2)c2ncnc(N3CCN(C(=O)OC(C)(C)C)C[C@@H]3C)c12] |
| 305 | 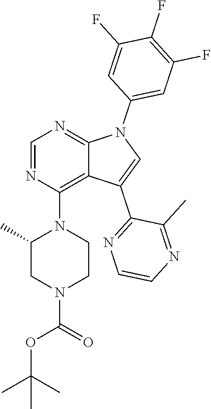 [SMILES: Cc1nccnc1-c1cn(-c2cc(F)c(F)c(F)c2)c2ncnc(N3CCN(C(=O)OC(C)(C)C)C[C@@H]3C)c12] |
| 306 | 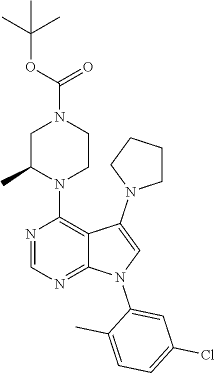 [SMILES: Cc1ccc(Cl)cc1-n1cc(N2CCCC2)c2c(N3CCN(C(=O)OC(C)(C)C)C[C@@H]3C)ncnc21] |
| 307 | 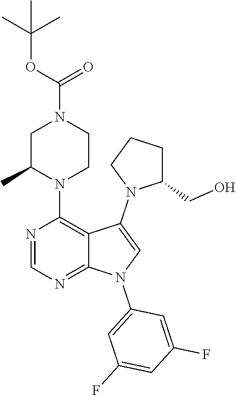 [SMILES: C[C@H]1CN(C(=O)OC(C)(C)C)CCN1c1ncnc2c1c(N1CCC[C@@H]1CO)cn2-c1cc(F)cc(F)c1] |
| 308 | 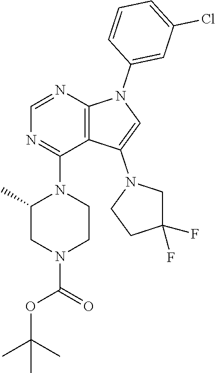 [SMILES: C[C@H]1CN(C(=O)OC(C)(C)C)CCN1c1ncnc2c1c(N1CCC(F)(F)C1)cn2-c1cccc(Cl)c1] |
| 309 | 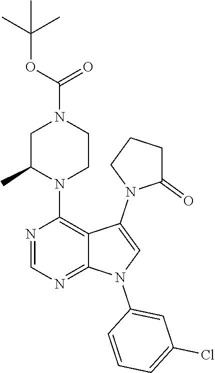 [SMILES: C[C@H]1CN(C(=O)OC(C)(C)C)CCN1c1ncnc2c1c(N1CCCC1=O)cn2-c1cccc(Cl)c1] |
| 310 | 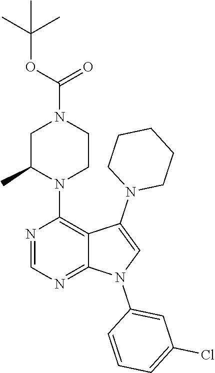 [SMILES: C[C@H]1CN(C(=O)OC(C)(C)C)CCN1c1ncnc2c1c(N1CCCCC1)cn2-c1cccc(Cl)c1] |
| 311 | 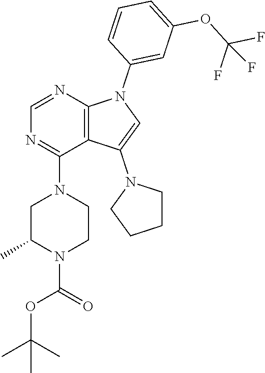 [SMILES: C[C@@H]1CN(c2ncnc3c2c(N2CCCC2)cn3-c2cccc(OC(F)(F)F)c2)CCN1C(=O)OC(C)(C)C] |
| 312 | 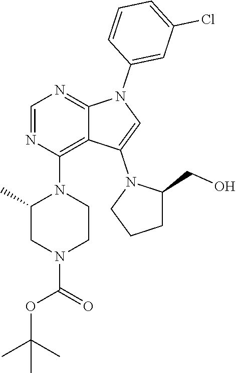 [SMILES: C[C@H]1CN(C(=O)OC(C)(C)C)CCN1c1ncnc2c1c(N1CCC[C@@H]1CO)cn2-c1cccc(Cl)c1] |
| 313 | 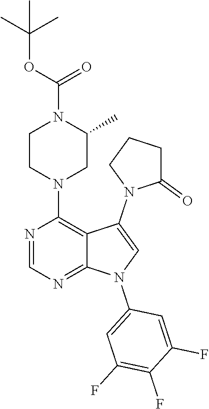 [SMILES: C[C@@H]1CN(c2ncnc3c2c(N2CCCC2=O)cn3-c2cc(F)c(F)c(F)c2)CCN1C(=O)OC(C)(C)C] |
| 314 | 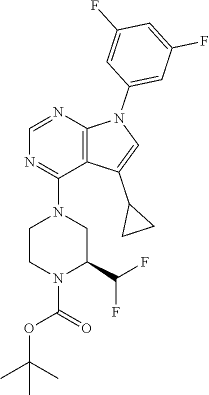 [SMILES: CC(C)(C)OC(=O)N1CCN(c2ncnc3c2c(C2CC2)cn3-c2cc(F)cc(F)c2)C[C@H]1C(F)F] |
| 315 | 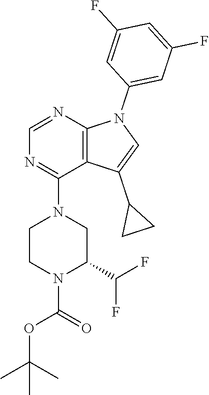 [SMILES: CC(C)(C)OC(=O)N1CCN(c2ncnc3c2c(C2CC2)cn3-c2cc(F)cc(F)c2)C[C@@H]1C(F)F] |
| 316 | 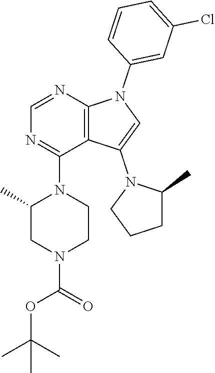 [SMILES: C[C@H]1CCCN1c1cn(-c2cccc(Cl)c2)c2ncnc(N3CCN(C(=O)OC(C)(C)C)C[C@@H]3C)c12] |
| 317 | 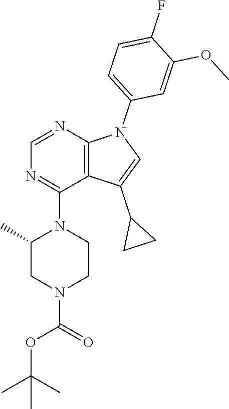 [SMILES: COc1cc(-n2cc(C3CC3)c3c(N4CCN(C(=O)OC(C)(C)C)C[C@@H]4C)ncnc32)ccc1F] |
| 318 | 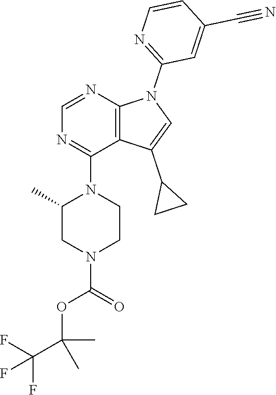 [SMILES: C[C@H]1CN(C(=O)OC(C)(C)C(F)(F)F)CCN1c1ncnc2c1c(C1CC1)cn2-c1cc(C#N)ccn1] |
| 319 | 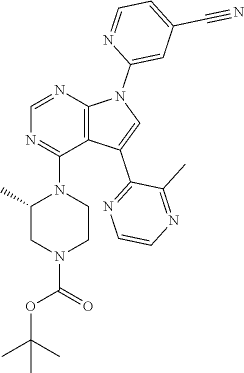 [SMILES: Cc1nccnc1-c1cn(-c2cc(C#N)ccn2)c2ncnc(N3CCN(C(=O)OC(C)(C)C)C[C@@H]3C)c12] |
| 320 | 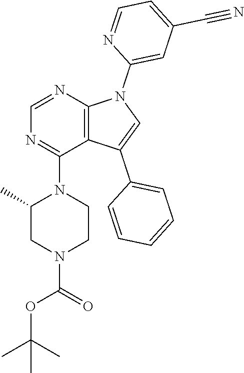 [SMILES: C[C@H]1CN(C(=O)OC(C)(C)C)CCN1c1ncnc2c1c(-c1ccccc1)cn2-c1cc(C#N)ccn1] |
| 321 | 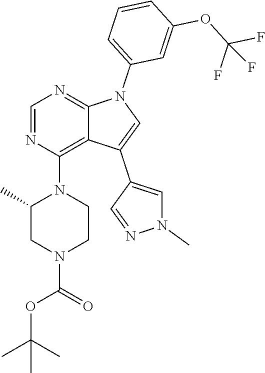 [SMILES: C[C@H]1CN(C(=O)OC(C)(C)C)CCN1c1ncnc2c1c(-c1cnn(C)c1)cn2-c1cccc(OC(F)(F)F)c1] |
| 322 | 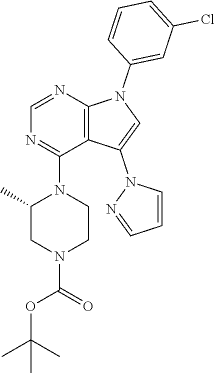 [SMILES: C[C@H]1CN(C(=O)OC(C)(C)C)CCN1c1ncnc2c1c(-n1cccn1)cn2-c1cccc(Cl)c1] |
| 323 | 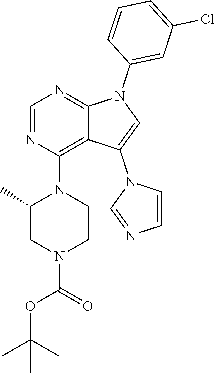 [SMILES: C[C@H]1CN(C(=O)OC(C)(C)C)CCN1c1ncnc2c1c(-n1ccnc1)cn2-c1cccc(Cl)c1] |
| 324 | 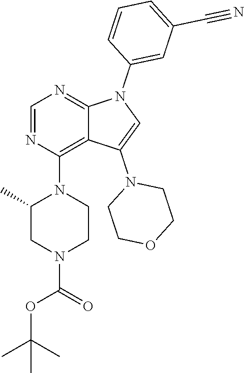 [SMILES: C[C@H]1CN(C(=O)OC(C)(C)C)CCN1c1ncnc2c1c(N1CCOCC1)cn2-c1cccc(C#N)c1] |
| 325 | 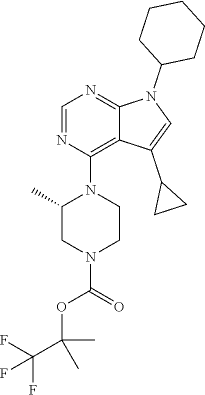 [SMILES: C[C@H]1CN(C(=O)OC(C)(C)C(F)(F)F)CCN1c1ncnc2c1c(C1CC1)cn2C1CCCCC1] |
| 326 | 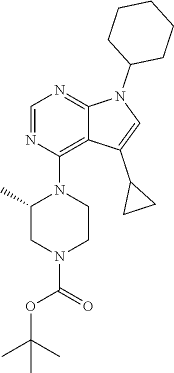 [SMILES: C[C@H]1CN(C(=O)OC(C)(C)C)CCN1c1ncnc2c1c(C1CC1)cn2C1CCCCC1] |
| 327 | 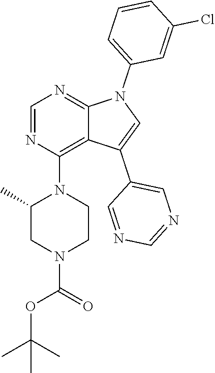 [SMILES: C[C@H]1CN(C(=O)OC(C)(C)C)CCN1c1ncnc2c1c(-c1cncnc1)cn2-c1cccc(Cl)c1] |
| 328 | 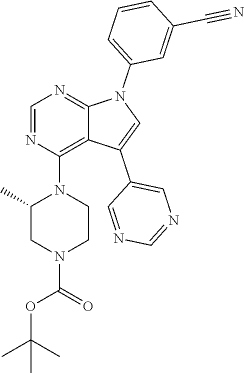 [SMILES: C[C@H]1CN(C(=O)OC(C)(C)C)CCN1c1ncnc2c1c(-c1cncnc1)cn2-c1cccc(C#N)c1] |
| 329 | 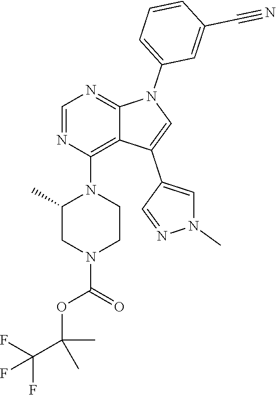 [SMILES: C[C@H]1CN(C(=O)OC(C)(C)C(F)(F)F)CCN1c1ncnc2c1c(-c1cnn(C)c1)cn2-c1cccc(C#N)c1] |
| 330 | 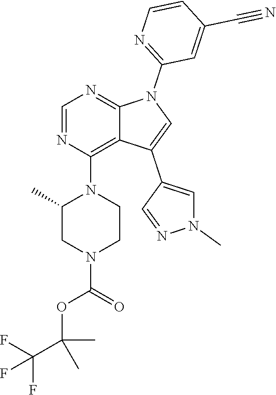 [SMILES: C[C@H]1CN(C(=O)OC(C)(C)C(F)(F)F)CCN1c1ncnc2c1c(-c1cnn(C)c1)cn2-c1cc(C#N)ccn1] |
| 331 | 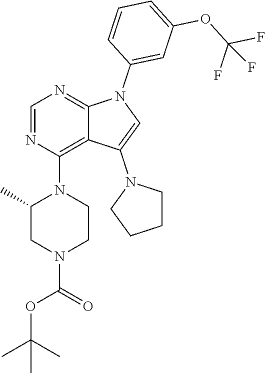 [SMILES: C[C@H]1CN(C(=O)OC(C)(C)C)CCN1c1ncnc2c1c(N1CCCC1)cn2-c1cccc(OC(F)(F)F)c1] |
| 332 | 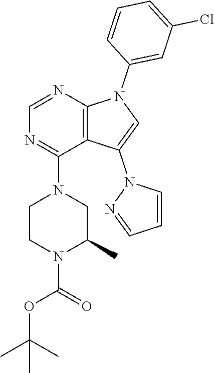 [SMILES: C[C@@H]1CN(c2ncnc3c2c(-n2cccn2)cn3-c2cccc(Cl)c2)CCN1C(=O)OC(C)(C)C] |
| 333 | 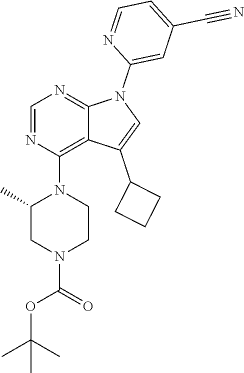 [SMILES: C[C@H]1CN(C(=O)OC(C)(C)C)CCN1c1ncnc2c1c(C1CCC1)cn2-c1cc(C#N)ccn1] |
| 334 | 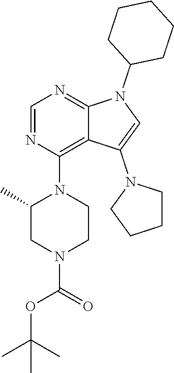 [SMILES: C[C@H]1CN(C(=O)OC(C)(C)C)CCN1c1ncnc2c1c(N1CCCC1)cn2C1CCCCC1] |
| 335 | 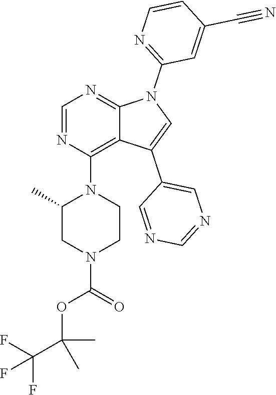 [SMILES: C[C@H]1CN(C(=O)OC(C)(C)C(F)(F)F)CCN1c1ncnc2c1c(-c1cncnc1)cn2-c1cc(C#N)ccn1] |
| 336 | 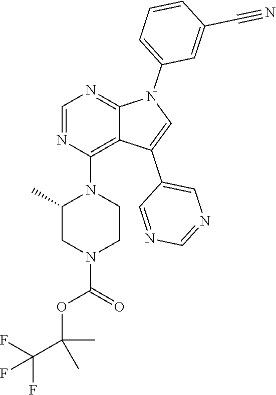 [SMILES: C[C@H]1CN(C(=O)OC(C)(C)C(F)(F)F)CCN1c1ncnc2c1c(-c1cncnc1)cn2-c1cccc(C#N)c1] |
| 337 | 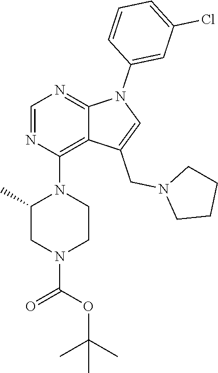 [SMILES: C[C@H]1CN(C(=O)OC(C)(C)C)CCN1c1ncnc2c1c(CN1CCCC1)cn2-c1cccc(Cl)c1] |
| 338 | 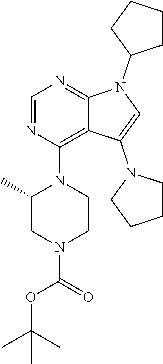 [SMILES: C[C@H]1CN(C(=O)OC(C)(C)C)CCN1c1ncnc2c1c(N1CCCC1)cn2C1CCCC1] |
| 339 | 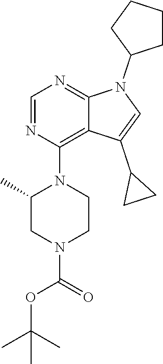 [SMILES: C[C@H]1CN(C(=O)OC(C)(C)C)CCN1c1ncnc2c1c(C1CC1)cn2C1CCCC1] |
| 340 | 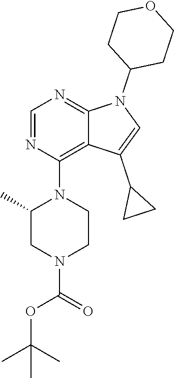 [SMILES: C[C@H]1CN(C(=O)OC(C)(C)C)CCN1c1ncnc2c1c(C1CC1)cn2C1CCOCC1] |
| 341 | 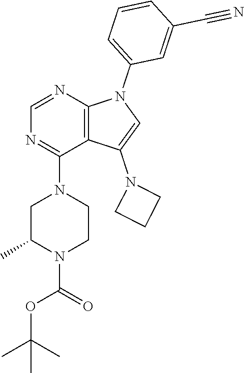 [SMILES: C[C@@H]1CN(c2ncnc3c2c(N2CCC2)cn3-c2cccc(C#N)c2)CCN1C(=O)OC(C)(C)C] |
| 342 | 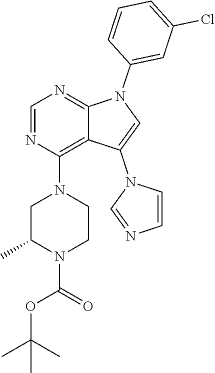 [SMILES: C[C@@H]1CN(c2ncnc3c2c(-n2ccnc2)cn3-c2cccc(Cl)c2)CCN1C(=O)OC(C)(C)C] |
| 343 | 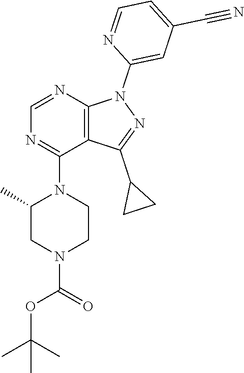 [SMILES: C[C@H]1CN(C(=O)OC(C)(C)C)CCN1c1ncnc2c1c(C1CC1)nn2-c1cc(C#N)ccn1] |
| 344 | 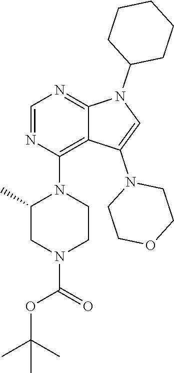 [SMILES: C[C@H]1CN(C(=O)OC(C)(C)C)CCN1c1ncnc2c1c(N1CCOCC1)cn2C1CCCCC1] |
| 345 | 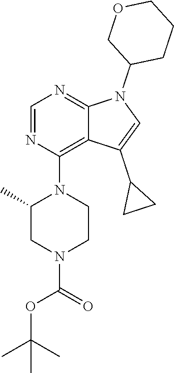 [SMILES: C[C@H]1CN(C(=O)OC(C)(C)C)CCN1c1ncnc2c1c(C1CC1)cn2C1CCCOC1] |
| 346 | 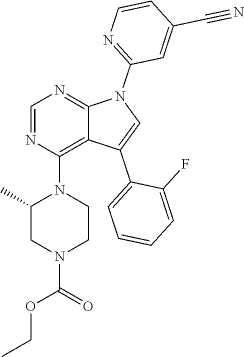 [SMILES: CCOC(=O)N1CCN(c2ncnc3c2c(-c2ccccc2F)cn3-c2cc(C#N)ccn2)[C@@H](C)C1] |
| 347 | 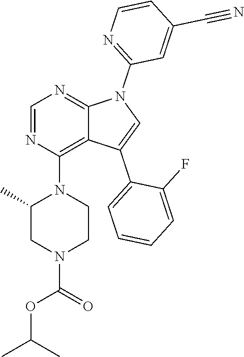 [SMILES: CC(C)OC(=O)N1CCN(c2ncnc3c2c(-c2ccccc2F)cn3-c2cc(C#N)ccn2)[C@@H](C)C1] |
| 348 | 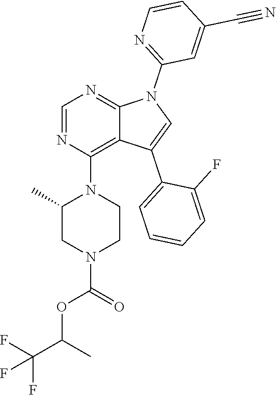 [SMILES: CC(OC(=O)N1CCN(c2ncnc3c2c(-c2ccccc2F)cn3-c2cc(C#N)ccn2)[C@@H](C)C1)C(F)(F)F] |
| 349 | 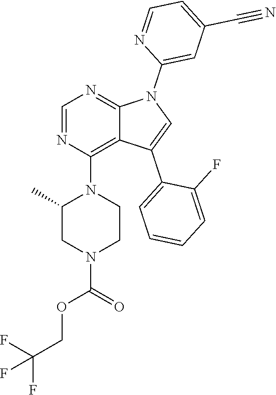 [SMILES: C[C@H]1CN(C(=O)OCC(F)(F)F)CCN1c1ncnc2c1c(-c1ccccc1F)cn2-c1cc(C#N)ccn1] |
| 350 | 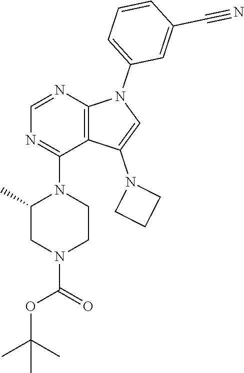 [SMILES: C[C@H]1CN(C(=O)OC(C)(C)C)CCN1c1ncnc2c1c(N1CCC1)cn2-c1cccc(C#N)c1] |
| 351 | 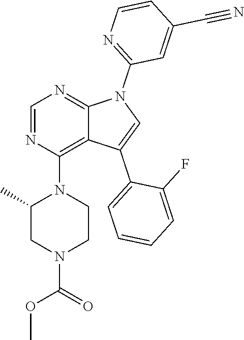 [SMILES: COC(=O)N1CCN(c2ncnc3c2c(-c2ccccc2F)cn3-c2cc(C#N)ccn2)[C@@H](C)C1] |
| 352 | 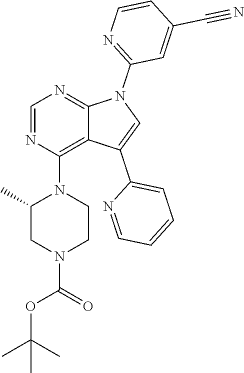 [SMILES: C[C@H]1CN(C(=O)OC(C)(C)C)CCN1c1ncnc2c1c(-c1ccccn1)cn2-c1cc(C#N)ccn1] |
| 353 | 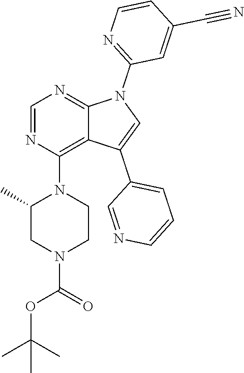 [SMILES: C[C@H]1CN(C(=O)OC(C)(C)C)CCN1c1ncnc2c1c(-c1cccnc1)cn2-c1cc(C#N)ccn1] |
| 354 | 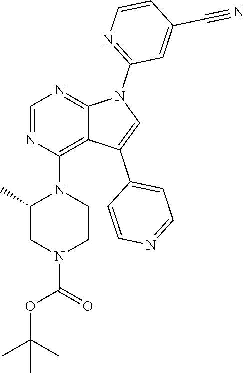 [SMILES: C[C@H]1CN(C(=O)OC(C)(C)C)CCN1c1ncnc2c1c(-c1ccncc1)cn2-c1cc(C#N)ccn1] |
| 355 | 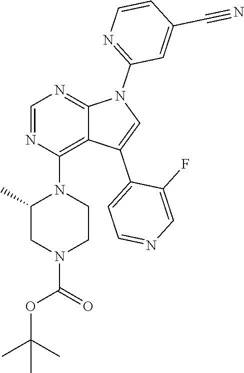 [SMILES: C[C@H]1CN(C(=O)OC(C)(C)C)CCN1c1ncnc2c1c(-c1ccncc1F)cn2-c1cc(C#N)ccn1] |
| 356 | 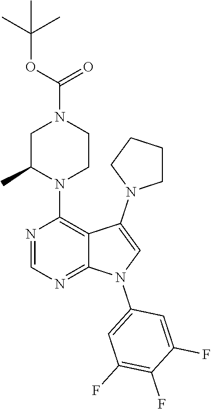 [SMILES: C[C@H]1CN(C(=O)OC(C)(C)C)CCN1c1ncnc2c1c(N1CCCC1)cn2-c1cc(F)c(F)c(F)c1] |
| 357 | 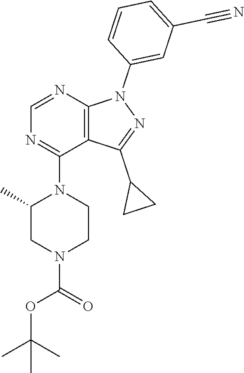 [SMILES: C[C@H]1CN(C(=O)OC(C)(C)C)CCN1c1ncnc2c1c(C1CC1)nn2-c1cccc(C#N)c1] |
| 358 | 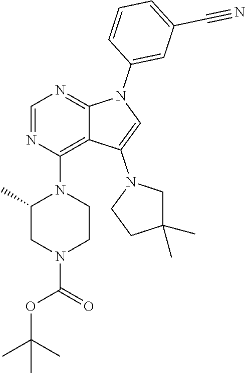 [SMILES: C[C@H]1CN(C(=O)OC(C)(C)C)CCN1c1ncnc2c1c(N1CCC(C)(C)C1)cn2-c1cccc(C#N)c1] |
| 359 | 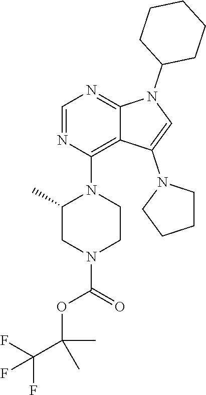 [SMILES: C[C@H]1CN(C(=O)OC(C)(C)C(F)(F)F)CCN1c1ncnc2c1c(N1CCCC1)cn2C1CCCCC1] |
| 360 | 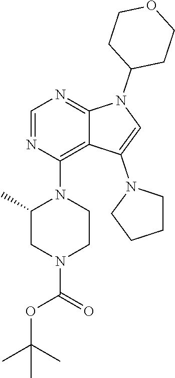 [SMILES: C[C@H]1CN(C(=O)OC(C)(C)C)CCN1c1ncnc2c1c(N1CCCC1)cn2C1CCOCC1] |
| 361 | 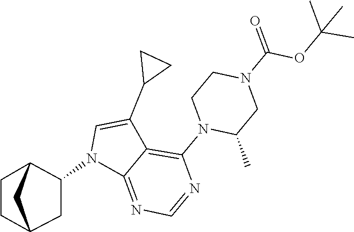 [SMILES: C[C@H]1CN(C(=O)OC(C)(C)C)CCN1c1ncnc2c1c(C1CC1)cn2[C@@H]1C[C@@H]2CC[C@H]1C2] |
| 362 | 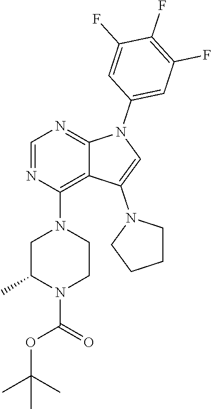 [SMILES: C[C@@H]1CN(c2ncnc3c2c(N2CCCC2)cn3-c2cc(F)c(F)c(F)c2)CCN1C(=O)OC(C)(C)C] |
| 363 | 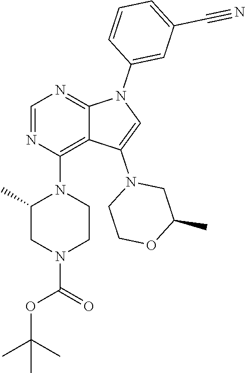 [SMILES: C[C@@H]1CN(c2cn(-c3cccc(C#N)c3)c3ncnc(N4CCN(C(=O)OC(C)(C)C)C[C@@H]4C)c23)CCO1] |
| 364 | 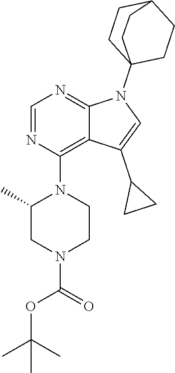 [SMILES: C[C@H]1CN(C(=O)OC(C)(C)C)CCN1c1ncnc2c1c(C1CC1)cn2C12CCC(CC1)CC2] |
| 365 | 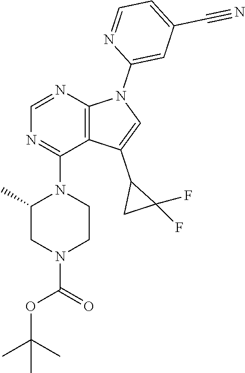 [SMILES: C[C@H]1CN(C(=O)OC(C)(C)C)CCN1c1ncnc2c1c(C1CC1(F)F)cn2-c1cc(C#N)ccn1] |
| 366 | 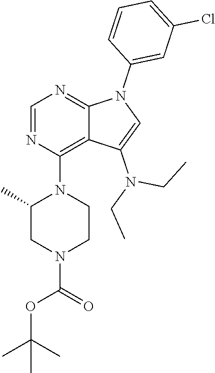 [SMILES: CCN(CC)c1cn(-c2cccc(Cl)c2)c2ncnc(N3CCN(C(=O)OC(C)(C)C)C[C@@H]3C)c12] |
| 367 | 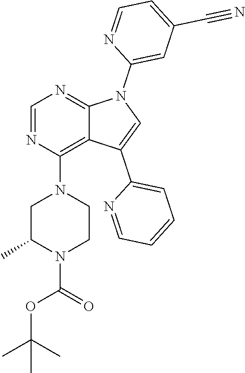 [SMILES: C[C@@H]1CN(c2ncnc3c2c(-c2ccccn2)cn3-c2cc(C#N)ccn2)CCN1C(=O)OC(C)(C)C] |
| 368 | 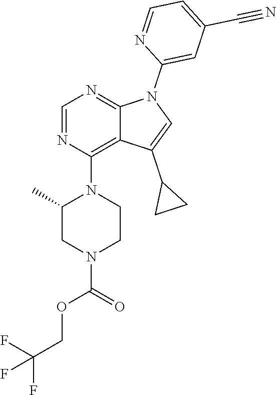 [SMILES: C[C@H]1CN(C(=O)OCC(F)(F)F)CCN1c1ncnc2c1c(C1CC1)cn2-c1cc(C#N)ccn1] |
| 369 | 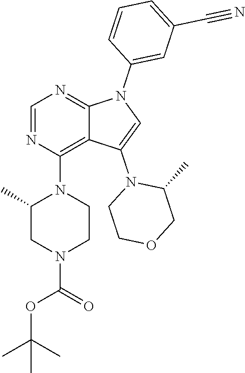 [SMILES: C[C@@H]1COCCN1c1cn(-c2cccc(C#N)c2)c2ncnc(N3CCN(C(=O)OC(C)(C)C)C[C@@H]3C)c12] |
| 370 | 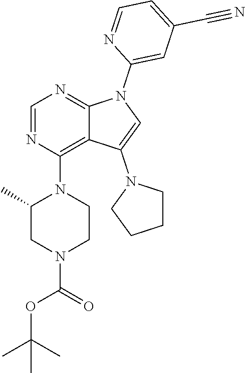 [SMILES: C[C@H]1CN(C(=O)OC(C)(C)C)CCN1c1ncnc2c1c(N1CCCC1)cn2-c1cc(C#N)ccn1] |
| 371 | 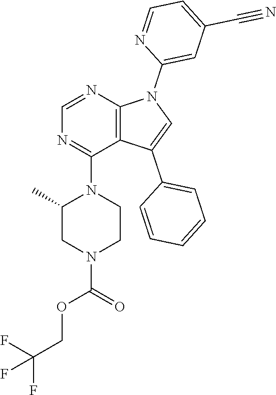 [SMILES: C[C@H]1CN(C(=O)OCC(F)(F)F)CCN1c1ncnc2c1c(-c1ccccc1)cn2-c1cc(C#N)ccn1] |
| 372 | 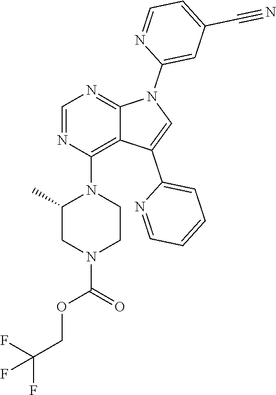 [SMILES: C[C@H]1CN(C(=O)OCC(F)(F)F)CCN1c1ncnc2c1c(-c1ccccn1)cn2-c1cc(C#N)ccn1] |
| 373 | 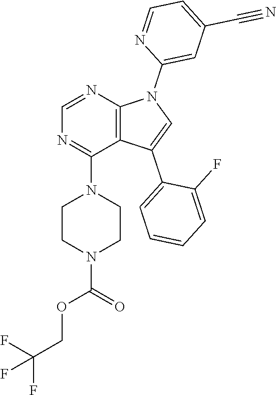 [SMILES: N#Cc1ccnc(-n2cc(-c3ccccc3F)c3c(N4CCN(C(=O)OCC(F)(F)F)CC4)ncnc32)c1] |
| 374 | 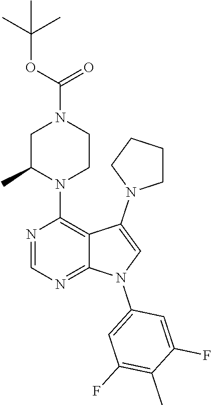 [SMILES: Cc1c(F)cc(-n2cc(N3CCCC3)c3c(N4CCN(C(=O)OC(C)(C)C)C[C@@H]4C)ncnc32)cc1F] |
| 375 | 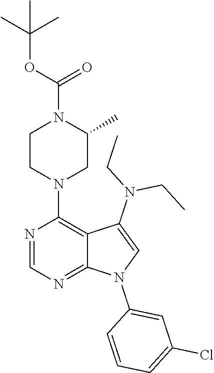 [SMILES: CCN(CC)c1cn(-c2cccc(Cl)c2)c2ncnc(N3CCN(C(=O)OC(C)(C)C)[C@H](C)C3)c12] |
| 376 | 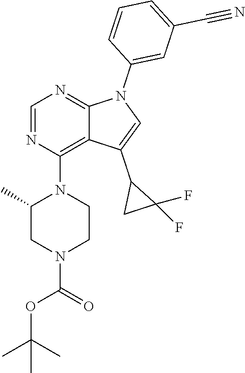 [SMILES: C[C@H]1CN(C(=O)OC(C)(C)C)CCN1c1ncnc2c1c(C1CC1(F)F)cn2-c1cccc(C#N)c1] |
| 377 | 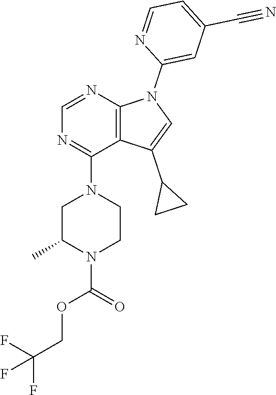 [SMILES: C[C@@H]1CN(c2ncnc3c2c(C2CC2)cn3-c2cc(C#N)ccn2)CCN1C(=O)OCC(F)(F)F] |
| 378 | 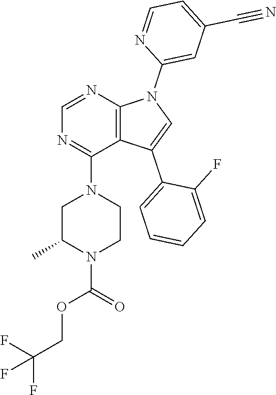 [SMILES: C[C@@H]1CN(c2ncnc3c2c(-c2ccccc2F)cn3-c2cc(C#N)ccn2)CCN1C(=O)OCC(F)(F)F] |
| 379 | 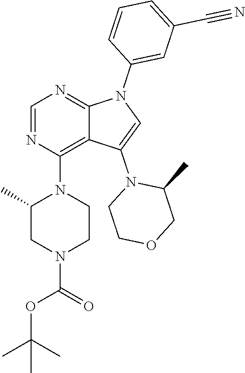 [SMILES: C[C@H]1COCCN1c1cn(-c2cccc(C#N)c2)c2ncnc(N3CCN(C(=O)OC(C)(C)C)C[C@@H]3C)c12] |
| 380 | 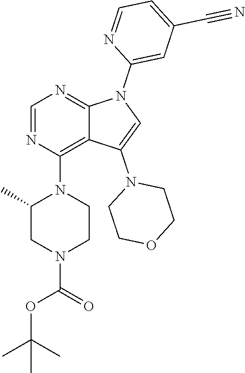 [SMILES: C[C@H]1CN(C(=O)OC(C)(C)C)CCN1c1ncnc2c1c(N1CCOCC1)cn2-c1cc(C#N)ccn1] |
| 381 | 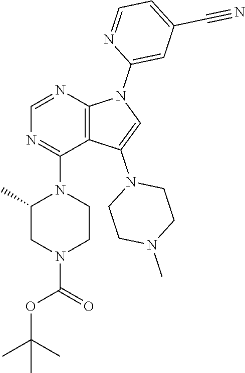 [SMILES: C[C@H]1CN(C(=O)OC(C)(C)C)CCN1c1ncnc2c1c(N1CCN(C)CC1)cn2-c1cc(C#N)ccn1] |
| 382 | 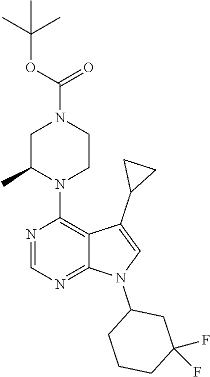 [SMILES: C[C@H]1CN(C(=O)OC(C)(C)C)CCN1c1ncnc2c1c(C1CC1)cn2C1CCCC(F)(F)C1] |
| 383 | 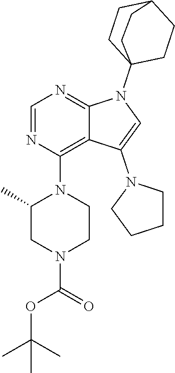 [SMILES: C[C@H]1CN(C(=O)OC(C)(C)C)CCN1c1ncnc2c1c(N1CCCC1)cn2C12CCC(CC1)CC2] |
| 384 | 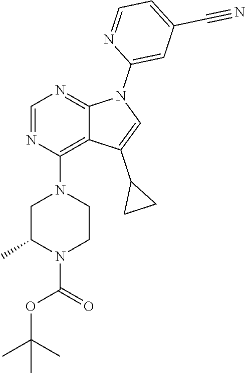 [SMILES: C[C@@H]1CN(c2ncnc3c2c(C2CC2)cn3-c2cc(C#N)ccn2)CCN1C(=O)OC(C)(C)C] |
| 385 | 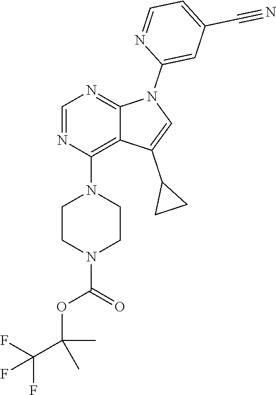 [SMILES: CC(C)(OC(=O)N1CCN(c2ncnc3c2c(C2CC2)cn3-c2cc(C#N)ccn2)CC1)C(F)(F)F] |
| 386 | 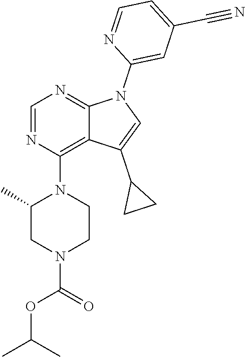 [SMILES: CC(C)OC(=O)N1CCN(c2ncnc3c2c(C2CC2)cn3-c2cc(C#N)ccn2)[C@@H](C)C1] |
| 387 | 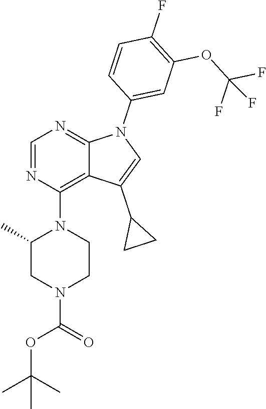 [SMILES: C[C@H]1CN(C(=O)OC(C)(C)C)CCN1c1ncnc2c1c(C1CC1)cn2-c1ccc(F)c(OC(F)(F)F)c1] |
| 388 | 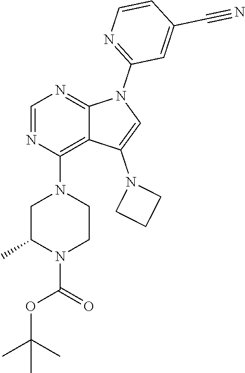 [SMILES: C[C@@H]1CN(c2ncnc3c2c(N2CCC2)cn3-c2cc(C#N)ccn2)CCN1C(=O)OC(C)(C)C] |
| 389 | 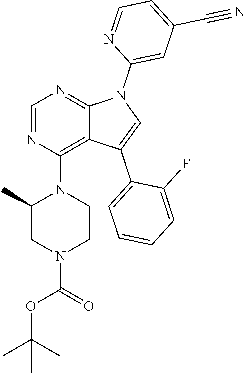 [SMILES: C[C@@H]1CN(C(=O)OC(C)(C)C)CCN1c1ncnc2c1c(-c1ccccc1F)cn2-c1cc(C#N)ccn1] |
| 390 | 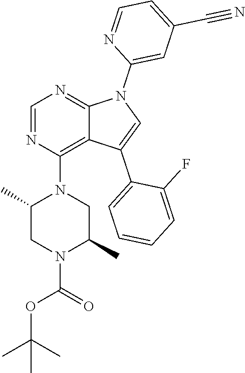 [SMILES: C[C@@H]1CN(c2ncnc3c2c(-c2ccccc2F)cn3-c2cc(C#N)ccn2)[C@@H](C)CN1C(=O)OC(C)(C)C] |
| 391 | 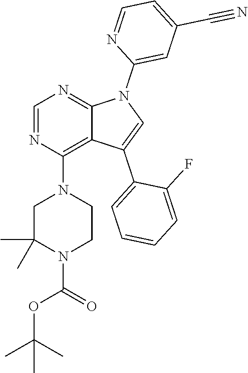 [SMILES: CC(C)(C)OC(=O)N1CCN(c2ncnc3c2c(-c2ccccc2F)cn3-c2cc(C#N)ccn2)CC1(C)C] |
| 392 | 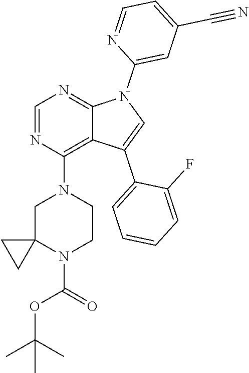 [SMILES: CC(C)(C)OC(=O)N1CCN(c2ncnc3c2c(-c2ccccc2F)cn3-c2cc(C#N)ccn2)CC12CC2] |
| 393 | 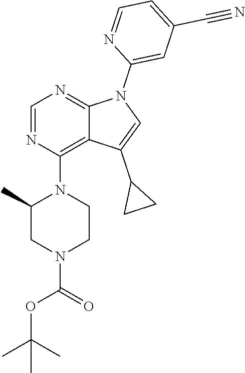 [SMILES: C[C@@H]1CN(C(=O)OC(C)(C)C)CCN1c1ncnc2c1c(C1CC1)cn2-c1cc(C#N)ccn1] |
| 394 | 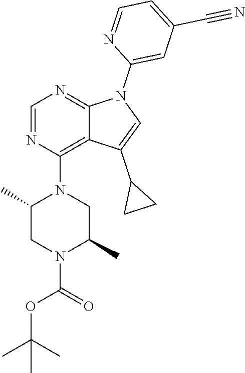 [SMILES: C[C@@H]1CN(c2ncnc3c2c(C2CC2)cn3-c2cc(C#N)ccn2)[C@@H](C)CN1C(=O)OC(C)(C)C] |
| 395 | 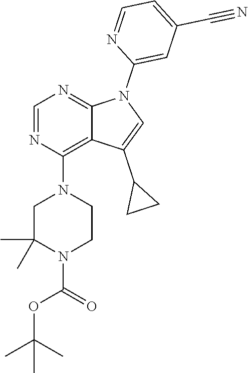 [SMILES: CC(C)(C)OC(=O)N1CCN(c2ncnc3c2c(C2CC2)cn3-c2cc(C#N)ccn2)CC1(C)C] |
| 396 | 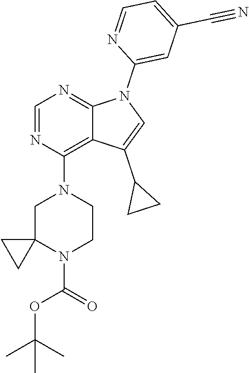 [SMILES: CC(C)(C)OC(=O)N1CCN(c2ncnc3c2c(C2CC2)cn3-c2cc(C#N)ccn2)CC12CC2] |
| 397 | 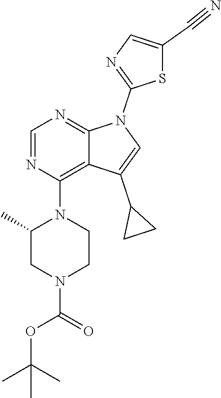 [SMILES: C[C@H]1CN(C(=O)OC(C)(C)C)CCN1c1ncnc2c1c(C1CC1)cn2-c1ncc(C#N)s1] |
| 398 | 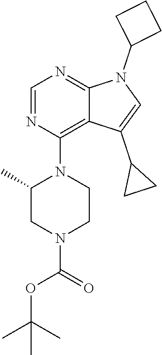 [SMILES: C[C@H]1CN(C(=O)OC(C)(C)C)CCN1c1ncnc2c1c(C1CC1)cn2C1CCC1] |
| 399 | 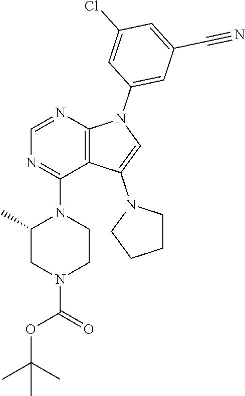 [SMILES: C[C@H]1CN(C(=O)OC(C)(C)C)CCN1c1ncnc2c1c(N1CCCC1)cn2-c1cc(Cl)cc(C#N)c1] |
| 400 | 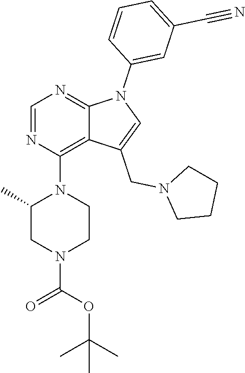 [SMILES: C[C@H]1CN(C(=O)OC(C)(C)C)CCN1c1ncnc2c1c(CN1CCCC1)cn2-c1cccc(C#N)c1] |
| 401 | 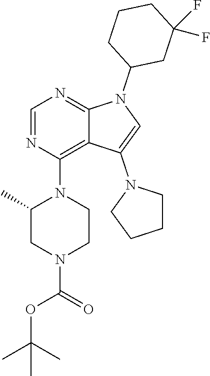 [SMILES: C[C@H]1CN(C(=O)OC(C)(C)C)CCN1c1ncnc2c1c(N1CCCC1)cn2C1CCCC(F)(F)C1] |
| 402 | 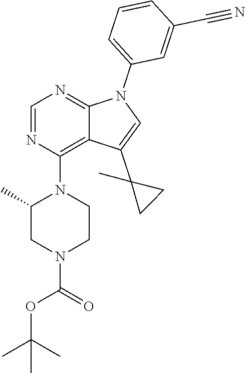 [SMILES: C[C@H]1CN(C(=O)OC(C)(C)C)CCN1c1ncnc2c1c(C1(C)CC1)cn2-c1cccc(C#N)c1] |
| 403 | 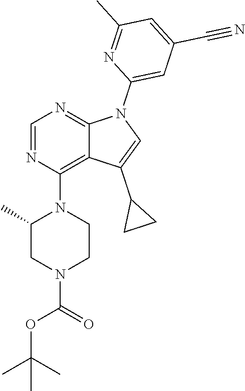 [SMILES: Cc1cc(C#N)cc(-n2cc(C3CC3)c3c(N4CCN(C(=O)OC(C)(C)C)C[C@@H]4C)ncnc32)n1] |
| 404 | 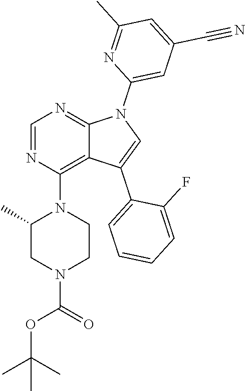 [SMILES: Cc1cc(C#N)cc(-n2cc(-c3ccccc3F)c3c(N4CCN(C(=O)OC(C)(C)C)C[C@@H]4C)ncnc32)n1] |
| 405 | 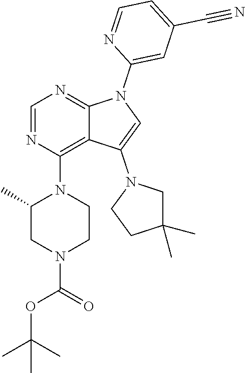 [SMILES: C[C@H]1CN(C(=O)OC(C)(C)C)CCN1c1ncnc2c1c(N1CCC(C)(C)C1)cn2-c1cc(C#N)ccn1] |
| 406 | 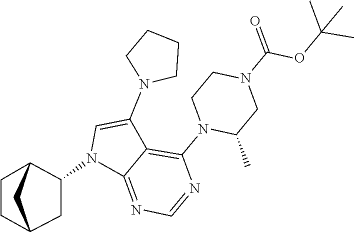 [SMILES: C[C@H]1CN(C(=O)OC(C)(C)C)CCN1c1ncnc2c1c(N1CCCC1)cn2[C@@H]1C[C@@H]2CC[C@H]1C2] |
| 407 | 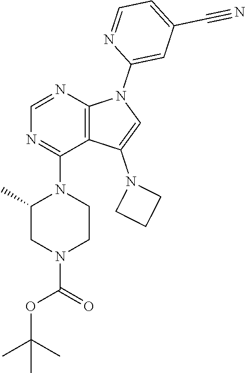 [SMILES: C[C@H]1CN(C(=O)OC(C)(C)C)CCN1c1ncnc2c1c(N1CCC1)cn2-c1cc(C#N)ccn1] |
| 408 | 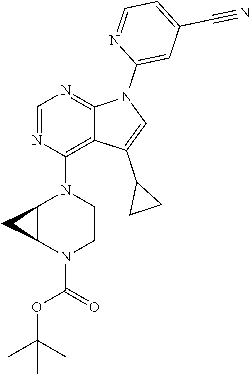 [SMILES: CC(C)(C)OC(=O)N1CCN(c2ncnc3c2c(C2CC2)cn3-c2cc(C#N)ccn2)[C@@H]2C[C@@H]21] |
| 409 | 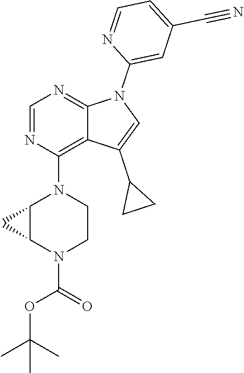 [SMILES: CC(C)(C)OC(=O)N1CCN(c2ncnc3c2c(C2CC2)cn3-c2cc(C#N)ccn2)[C@H]2C[C@H]21] |
| 410 | 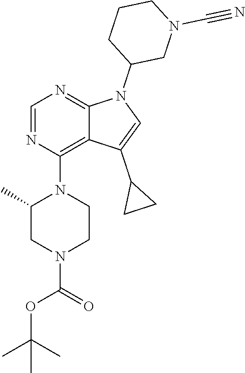 [SMILES: C[C@H]1CN(C(=O)OC(C)(C)C)CCN1c1ncnc2c1c(C1CC1)cn2C1CCCN(C#N)C1] |
| 411 | 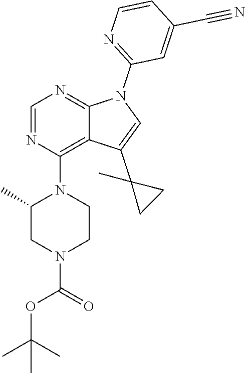 [SMILES: C[C@H]1CN(C(=O)OC(C)(C)C)CCN1c1ncnc2c1c(C1(C)CC1)cn2-c1cc(C#N)ccn1] |
| 412 | 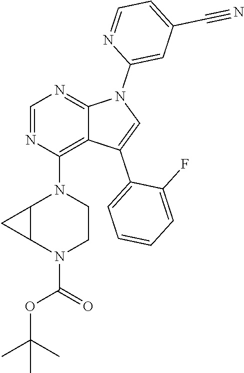 [SMILES: CC(C)(C)OC(=O)N1CCN(c2ncnc3c2c(-c2ccccc2F)cn3-c2cc(C#N)ccn2)C2CC21] |
| 413 | 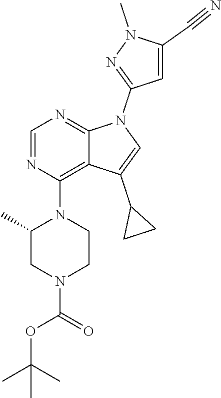 [SMILES: C[C@H]1CN(C(=O)OC(C)(C)C)CCN1c1ncnc2c1c(C1CC1)cn2-c1cc(C#N)n(C)n1] |
| 414 | 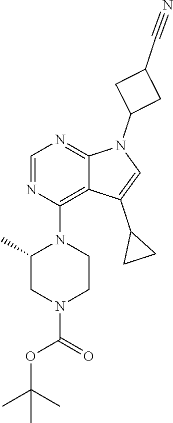 [SMILES: C[C@H]1CN(C(=O)OC(C)(C)C)CCN1c1ncnc2c1c(C1CC1)cn2C1CC(C#N)C1] |
| 415 | 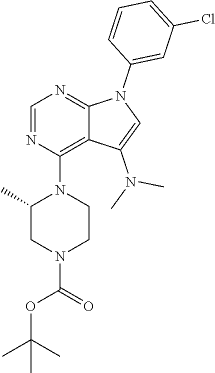 [SMILES: C[C@H]1CN(C(=O)OC(C)(C)C)CCN1c1ncnc2c1c(N(C)C)cn2-c1cccc(Cl)c1] |
| 416 | 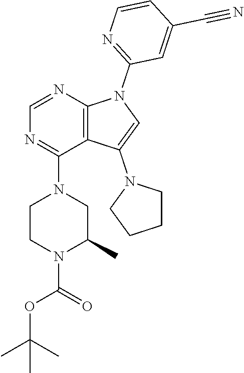 [SMILES: C[C@@H]1CN(c2ncnc3c2c(N2CCCC2)cn3-c2cc(C#N)ccn2)CCN1C(=O)OC(C)(C)C] |
| 417 | 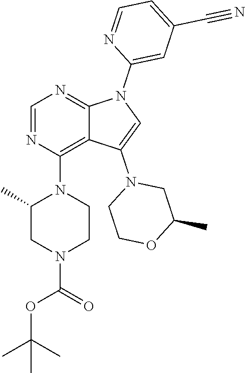 [SMILES: C[C@@H]1CN(c2cn(-c3cc(C#N)ccn3)c3ncnc(N4CCN(C(=O)OC(C)(C)C)C[C@@H]4C)c23)CCO1] |
| 418 | 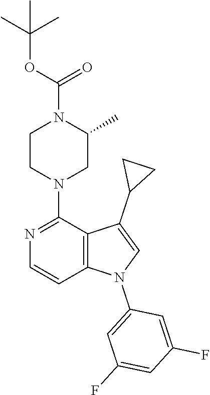 [SMILES: C[C@@H]1CN(c2nccc3c2c(C2CC2)cn3-c2cc(F)cc(F)c2)CCN1C(=O)OC(C)(C)C] |
| 419 | 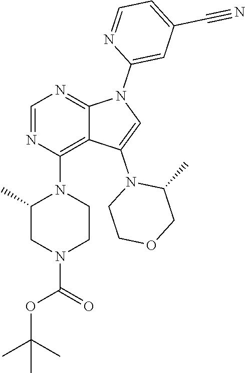 [SMILES: C[C@@H]1COCCN1c1cn(-c2cc(C#N)ccn2)c2ncnc(N3CCN(C(=O)OC(C)(C)C)C[C@@H]3C)c12] |
| 420 | 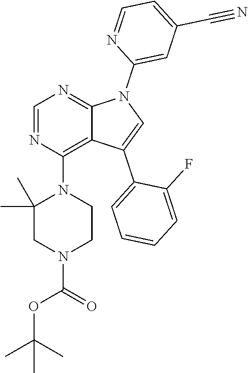 [SMILES: CC(C)(C)OC(=O)N1CCN(c2ncnc3c2c(-c2ccccc2F)cn3-c2cc(C#N)ccn2)C(C)(C)C1] |
| 421 | 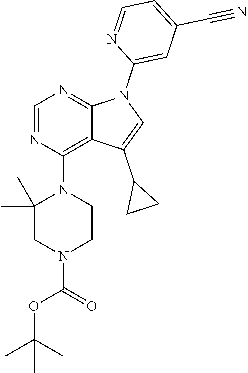 [SMILES: CC(C)(C)OC(=O)N1CCN(c2ncnc3c2c(C2CC2)cn3-c2cc(C#N)ccn2)C(C)(C)C1] |
| 422 | 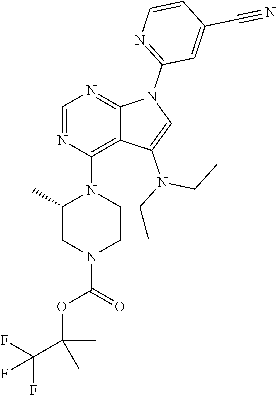 [SMILES: CCN(CC)c1cn(-c2cc(C#N)ccn2)c2ncnc(N3CCN(C(=O)OC(C)(C)C(F)(F)F)C[C@@H]3C)c12] |
| 423 | 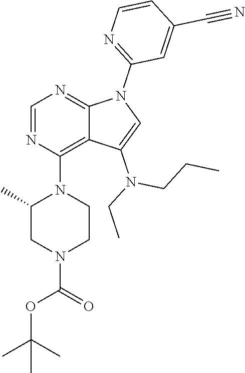 [SMILES: CCCN(CC)c1cn(-c2cc(C#N)ccn2)c2ncnc(N3CCN(C(=O)OC(C)(C)C)C[C@@H]3C)c12] |
| 424 | 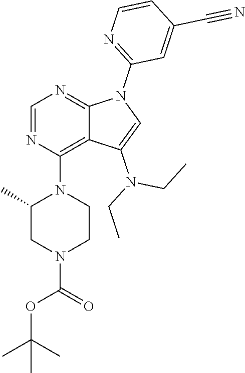 [SMILES: CCN(CC)c1cn(-c2cc(C#N)ccn2)c2ncnc(N3CCN(C(=O)OC(C)(C)C)C[C@@H]3C)c12] |
| 425 | 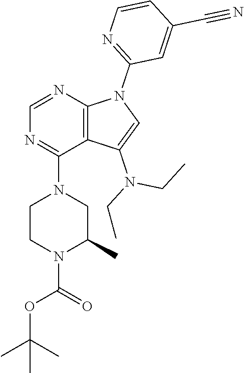 [SMILES: CCN(CC)c1cn(-c2cc(C#N)ccn2)c2ncnc(N3CCN(C(=O)OC(C)(C)C)[C@H](C)C3)c12] |
| 426 | 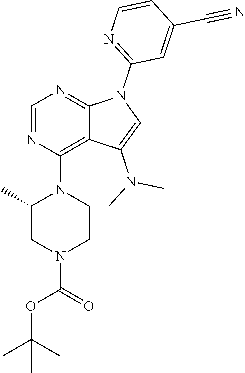 [SMILES: C[C@H]1CN(C(=O)OC(C)(C)C)CCN1c1ncnc2c1c(N(C)C)cn2-c1cc(C#N)ccn1] |
| 427 | 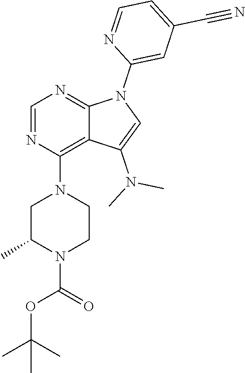 [SMILES: C[C@@H]1CN(c2ncnc3c2c(N(C)C)cn3-c2cc(C#N)ccn2)CCN1C(=O)OC(C)(C)C] |
| 428 | 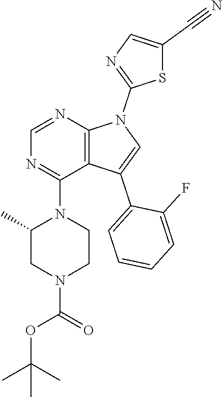 [SMILES: C[C@H]1CN(C(=O)OC(C)(C)C)CCN1c1ncnc2c1c(-c1ccccc1F)cn2-c1ncc(C#N)s1] |
| 429 | 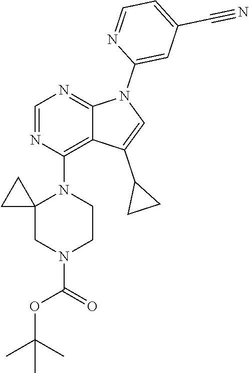 [SMILES: CC(C)(C)OC(=O)N1CCN(c2ncnc3c2c(C2CC2)cn3-c2cc(C#N)ccn2)C2(CC2)C1] |
| 430 | 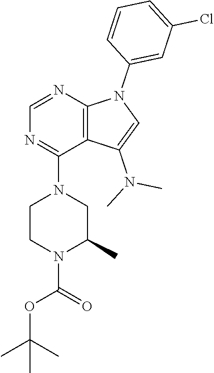 [SMILES: C[C@@H]1CN(c2ncnc3c2c(N(C)C)cn3-c2cccc(Cl)c2)CCN1C(=O)OC(C)(C)C] |
| 431 | 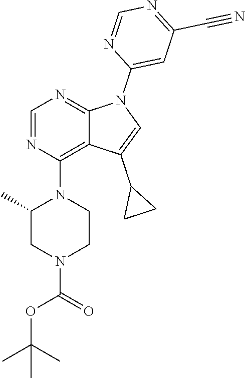 [SMILES: C[C@H]1CN(C(=O)OC(C)(C)C)CCN1c1ncnc2c1c(C1CC1)cn2-c1cc(C#N)ncn1] |
| 432 | 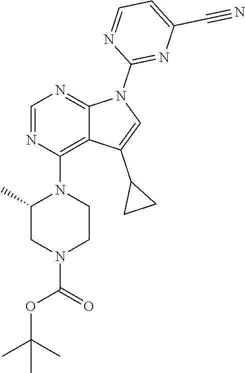 [SMILES: C[C@H]1CN(C(=O)OC(C)(C)C)CCN1c1ncnc2c1c(C1CC1)cn2-c1nccc(C#N)n1] |
| 433 | 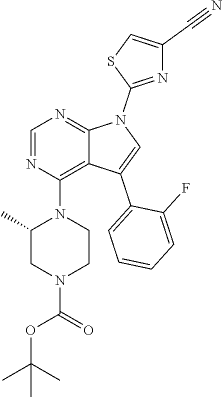 [SMILES: C[C@H]1CN(C(=O)OC(C)(C)C)CCN1c1ncnc2c1c(-c1ccccc1F)cn2-c1nc(C#N)cs1] |
| 434 | 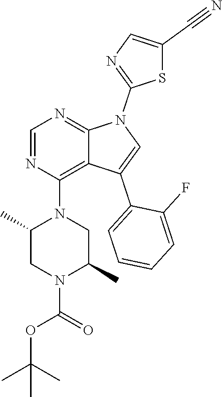 [SMILES: C[C@@H]1CN(c2ncnc3c2c(-c2ccccc2F)cn3-c2ncc(C#N)s2)[C@@H](C)CN1C(=O)OC(C)(C)C] |
| 435 | 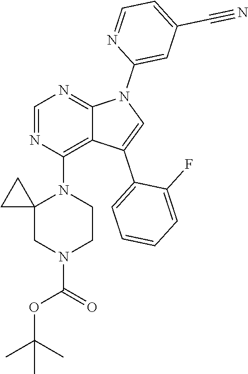 [SMILES: CC(C)(C)OC(=O)N1CCN(c2ncnc3c2c(-c2ccccc2F)cn3-c2cc(C#N)ccn2)C2(CC2)C1] |
| 436 | 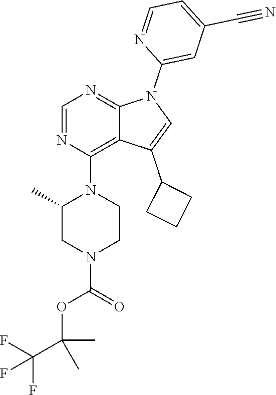 [SMILES: C[C@H]1CN(C(=O)OC(C)(C)C(F)(F)F)CCN1c1ncnc2c1c(C1CCC1)cn2-c1cc(C#N)ccn1] |
| 437 | 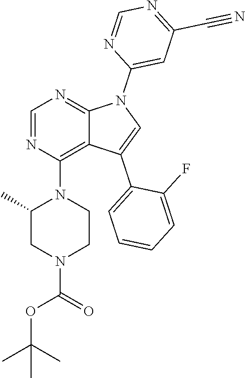 [SMILES: C[C@H]1CN(C(=O)OC(C)(C)C)CCN1c1ncnc2c1c(-c1ccccc1F)cn2-c1cc(C#N)ncn1] |
| 438 | 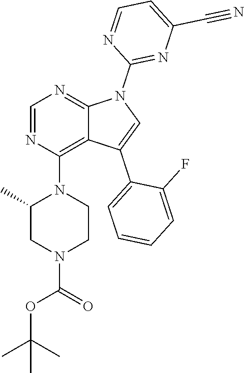 [SMILES: C[C@H]1CN(C(=O)OC(C)(C)C)CCN1c1ncnc2c1c(-c1ccccc1F)cn2-c1nccc(C#N)n1] |
| 439 | 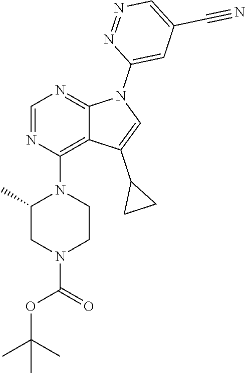 [SMILES: C[C@H]1CN(C(=O)OC(C)(C)C)CCN1c1ncnc2c1c(C1CC1)cn2-c1cc(C#N)cnn1] |
| 440 | 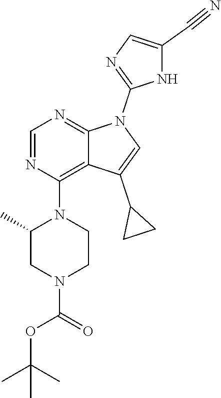 [SMILES: C[C@H]1CN(C(=O)OC(C)(C)C)CCN1c1ncnc2c1c(C1CC1)cn2-c1ncc(C#N)[nH]1] |
| 441 | 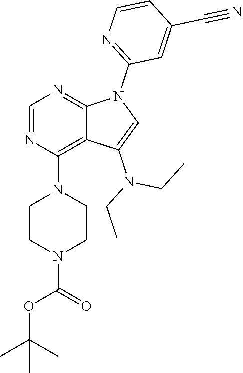 [SMILES: CCN(CC)c1cn(-c2cc(C#N)ccn2)c2ncnc(N3CCN(C(=O)OC(C)(C)C)CC3)c12] |
| 442 | 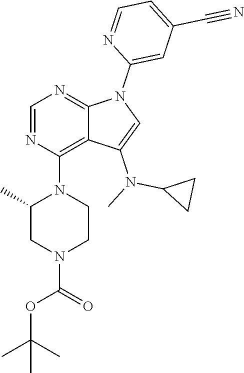 [SMILES: C[C@H]1CN(C(=O)OC(C)(C)C)CCN1c1ncnc2c1c(N(C)C1CC1)cn2-c1cc(C#N)ccn1] |
| 443 | 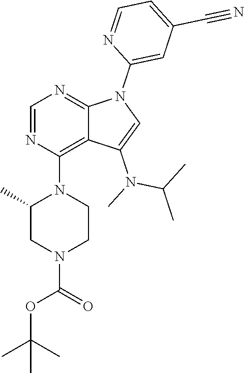 [SMILES: CC(C)N(C)c1cn(-c2cc(C#N)ccn2)c2ncnc(N3CCN(C(=O)OC(C)(C)C)C[C@@H]3C)c12] |
| 444 | 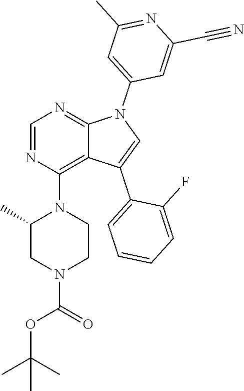 [SMILES: Cc1cc(-n2cc(-c3ccccc3F)c3c(N4CCN(C(=O)OC(C)(C)C)C[C@@H]4C)ncnc32)cc(C#N)n1] |
| 445 | 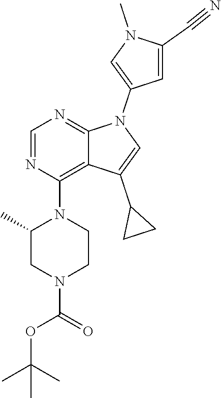 [SMILES: C[C@H]1CN(C(=O)OC(C)(C)C)CCN1c1ncnc2c1c(C1CC1)cn2-c1cc(C#N)n(C)c1] |
| 446 | 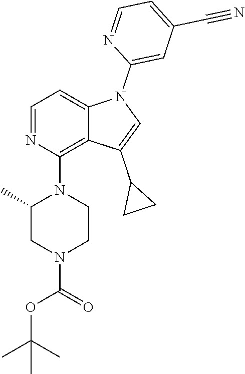 [SMILES: C[C@H]1CN(C(=O)OC(C)(C)C)CCN1c1nccc2c1c(C1CC1)cn2-c1cc(C#N)ccn1] |
| 447 | 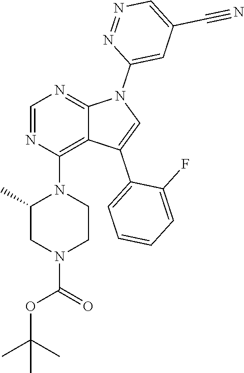 [SMILES: C[C@H]1CN(C(=O)OC(C)(C)C)CCN1c1ncnc2c1c(-c1ccccc1F)cn2-c1cc(C#N)cnn1] |
| 448 | 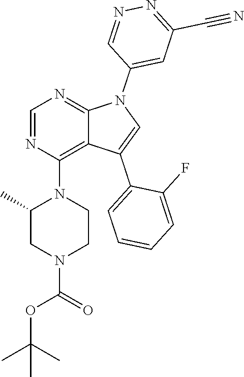 [SMILES: C[C@H]1CN(C(=O)OC(C)(C)C)CCN1c1ncnc2c1c(-c1ccccc1F)cn2-c1cnnc(C#N)c1] |
| 449 | 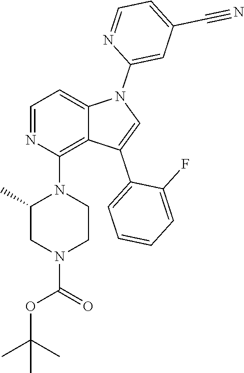 [SMILES: C[C@H]1CN(C(=O)OC(C)(C)C)CCN1c1nccc2c1c(-c1ccccc1F)cn2-c1cc(C#N)ccn1] |
| 450 | 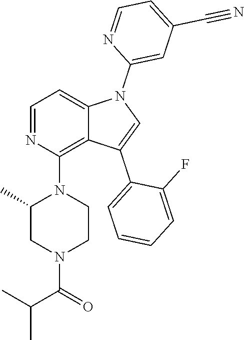 [SMILES: CC(C)C(=O)N1CCN(c2nccc3c2c(-c2ccccc2F)cn3-c2cc(C#N)ccn2)[C@@H](C)C1] |
| 451 | 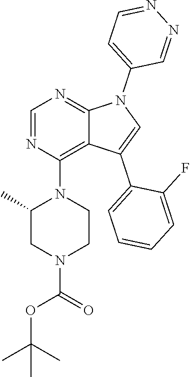 [SMILES: C[C@H]1CN(C(=O)OC(C)(C)C)CCN1c1ncnc2c1c(-c1ccccc1F)cn2-c1ccnnc1] |
| 452 | 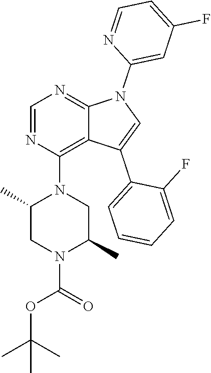 [SMILES: C[C@@H]1CN(c2ncnc3c2c(-c2ccccc2F)cn3-c2cc(F)ccn2)[C@@H](C)CN1C(=O)OC(C)(C)C] |
| 453 | 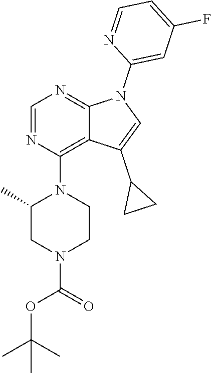 [SMILES: C[C@H]1CN(C(=O)OC(C)(C)C)CCN1c1ncnc2c1c(C1CC1)cn2-c1cc(F)ccn1] |
| 454 | 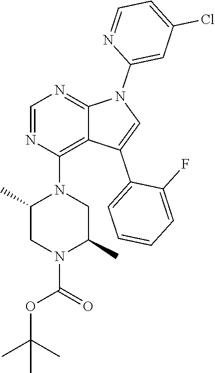 [SMILES: C[C@@H]1CN(c2ncnc3c2c(-c2ccccc2F)cn3-c2cc(Cl)ccn2)[C@@H](C)CN1C(=O)OC(C)(C)C] |
| 455 | 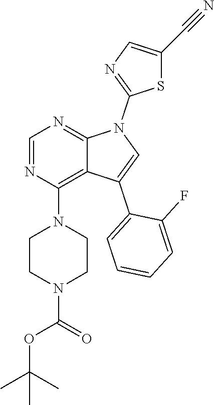 [SMILES: CC(C)(C)OC(=O)N1CCN(c2ncnc3c2c(-c2ccccc2F)cn3-c2ncc(C#N)s2)CC1] |
| 456 | 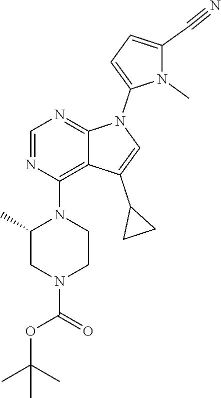 [SMILES: C[C@H]1CN(C(=O)OC(C)(C)C)CCN1c1ncnc2c1c(C1CC1)cn2-c1ccc(C#N)n1C] |
| 457 | 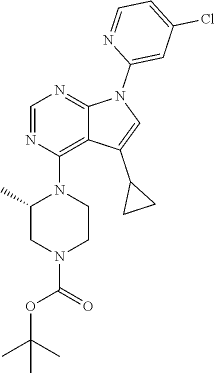 [SMILES: C[C@H]1CN(C(=O)OC(C)(C)C)CCN1c1ncnc2c1c(C1CC1)cn2-c1cc(Cl)ccn1] |
| 458 | 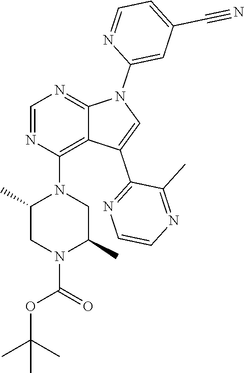 [SMILES: Cc1nccnc1-c1cn(-c2cc(C#N)ccn2)c2ncnc(N3C[C@@H](C)N(C(=O)OC(C)(C)C)C[C@@H]3C)c12] |
| 459 | 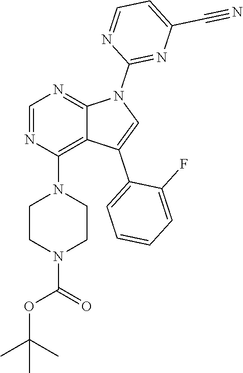 [SMILES: CC(C)(C)OC(=O)N1CCN(c2ncnc3c2c(-c2ccccc2F)cn3-c2nccc(C#N)n2)CC1] |
| 460 | 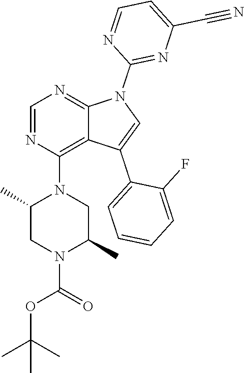 [SMILES: C[C@@H]1CN(c2ncnc3c2c(-c2ccccc2F)cn3-c2nccc(C#N)n2)[C@@H](C)CN1C(=O)OC(C)(C)C] |
| 461 | 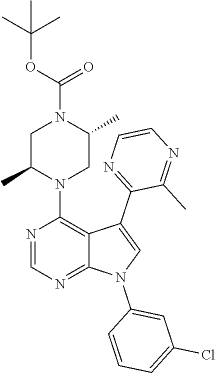 [SMILES: Cc1nccnc1-c1cn(-c2cccc(Cl)c2)c2ncnc(N3C[C@@H](C)N(C(=O)OC(C)(C)C)C[C@@H]3C)c12] |
| 462 | 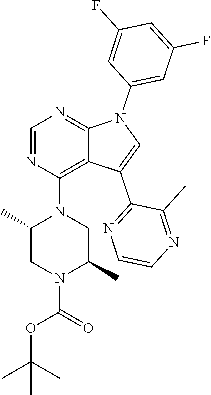 [SMILES: Cc1nccnc1-c1cn(-c2cc(F)cc(F)c2)c2ncnc(N3C[C@@H](C)N(C(=O)OC(C)(C)C)C[C@@H]3C)c12] |
| 463 | 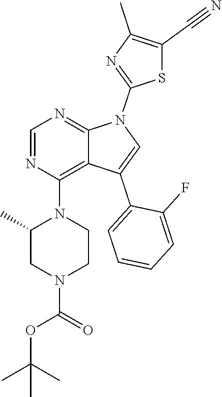 [SMILES: Cc1nc(-n2cc(-c3ccccc3F)c3c(N4CCN(C(=O)OC(C)(C)C)C[C@@H]4C)ncnc32)sc1C#N] |
| 464 | 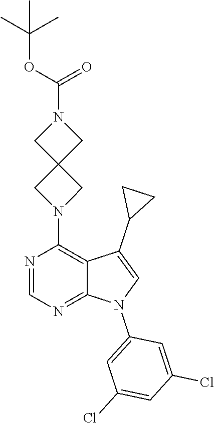 [SMILES: CC(C)(C)OC(=O)N1CC2(C1)CN(c1ncnc3c1c(C1CC1)cn3-c1cc(Cl)cc(Cl)c1)C2] |
| 465 | 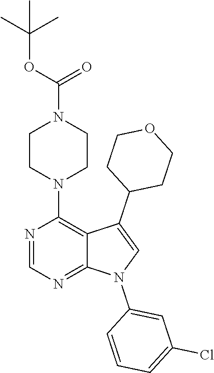 [SMILES: CC(C)(C)OC(=O)N1CCN(c2ncnc3c2c(C2CCOCC2)cn3-c2cccc(Cl)c2)CC1] |
| 466 | 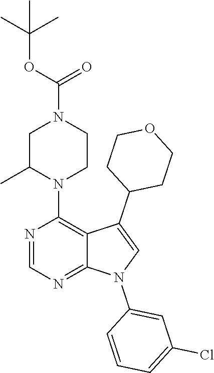 [SMILES: CC1CN(C(=O)OC(C)(C)C)CCN1c1ncnc2c1c(C1CCOCC1)cn2-c1cccc(Cl)c1] |
| 467 | 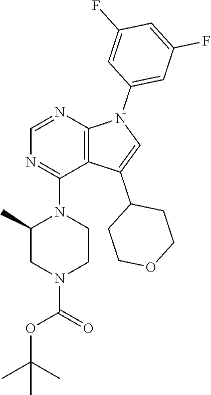 [SMILES: C[C@@H]1CN(C(=O)OC(C)(C)C)CCN1c1ncnc2c1c(C1CCOCC1)cn2-c1cc(F)cc(F)c1] |
| 468 | 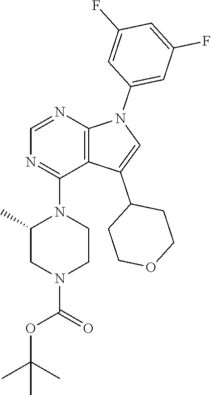 [SMILES: C[C@H]1CN(C(=O)OC(C)(C)C)CCN1c1ncnc2c1c(C1CCOCC1)cn2-c1cc(F)cc(F)c1] |
| 469 | 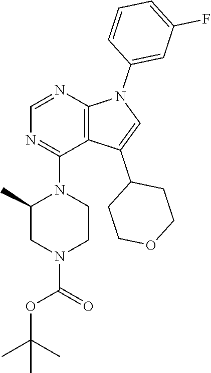 [SMILES: C[C@@H]1CN(C(=O)OC(C)(C)C)CCN1c1ncnc2c1c(C1CCOCC1)cn2-c1cccc(F)c1] |
| 470 | 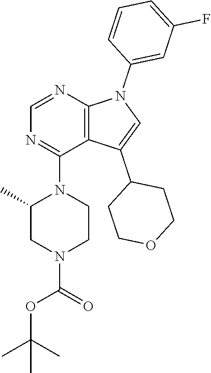 [SMILES: C[C@H]1CN(C(=O)OC(C)(C)C)CCN1c1ncnc2c1c(C1CCOCC1)cn2-c1cccc(F)c1] |
| 471 | 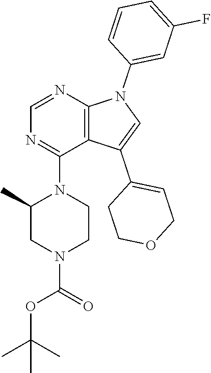 [SMILES: C[C@@H]1CN(C(=O)OC(C)(C)C)CCN1c1ncnc2c1c(C1=CCOCC1)cn2-c1cccc(F)c1] |
| 472 | 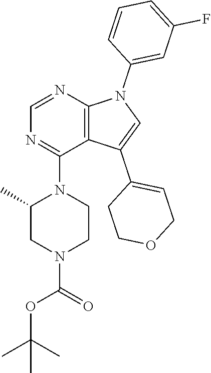 [SMILES: C[C@H]1CN(C(=O)OC(C)(C)C)CCN1c1ncnc2c1c(C1=CCOCC1)cn2-c1cccc(F)c1] |
| 473 | 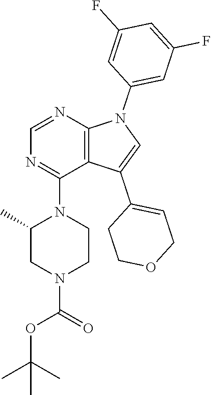 [SMILES: C[C@H]1CN(C(=O)OC(C)(C)C)CCN1c1ncnc2c1c(C1=CCOCC1)cn2-c1cc(F)cc(F)c1] |
| 474 | 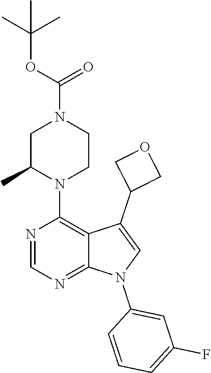 [SMILES: C[C@H]1CN(C(=O)OC(C)(C)C)CCN1c1ncnc2c1c(C1COC1)cn2-c1cccc(F)c1] |
| 475 | 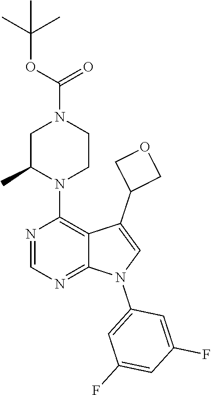 [SMILES: C[C@H]1CN(C(=O)OC(C)(C)C)CCN1c1ncnc2c1c(C1COC1)cn2-c1cc(F)cc(F)c1] |
| 476 | 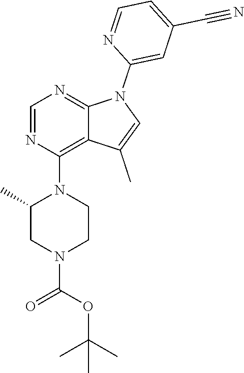 [SMILES: Cc1cn(-c2cc(C#N)ccn2)c2ncnc(N3CCN(C(=O)OC(C)(C)C)C[C@@H]3C)c12] |
| 477 | 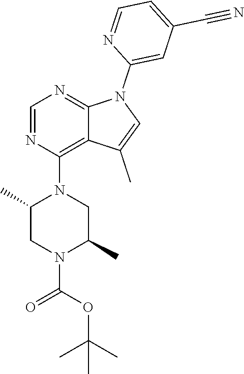 [SMILES: Cc1cn(-c2cc(C#N)ccn2)c2ncnc(N3C[C@@H](C)N(C(=O)OC(C)(C)C)C[C@@H]3C)c12] |
| 478 | 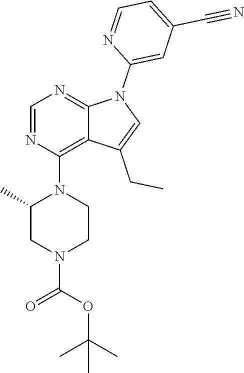 [SMILES: CCc1cn(-c2cc(C#N)ccn2)c2ncnc(N3CCN(C(=O)OC(C)(C)C)C[C@@H]3C)c12] |
| 479 | 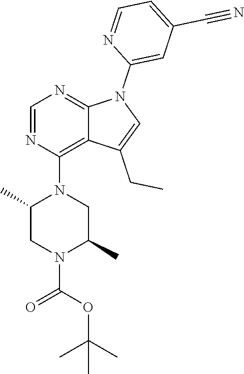 [SMILES: CCc1cn(-c2cc(C#N)ccn2)c2ncnc(N3C[C@@H](C)N(C(=O)OC(C)(C)C)C[C@@H]3C)c12] |
| 480 | 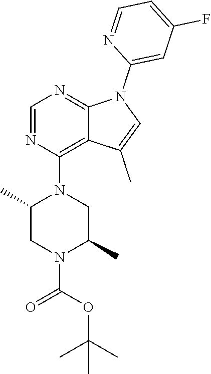 [SMILES: Cc1cn(-c2cc(F)ccn2)c2ncnc(N3C[C@@H](C)N(C(=O)OC(C)(C)C)C[C@@H]3C)c12] |
| 481 | 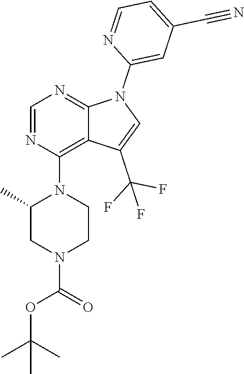 [SMILES: C[C@H]1CN(C(=O)OC(C)(C)C)CCN1c1ncnc2c1c(C(F)(F)F)cn2-c1cc(C#N)ccn1] |
| 482 | 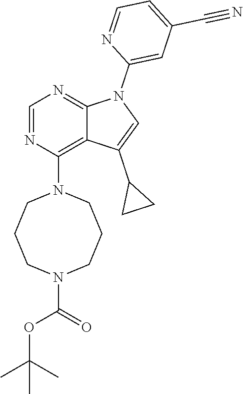 [SMILES: CC(C)(C)OC(=O)N1CCCN(c2ncnc3c2c(C2CC2)cn3-c2cc(C#N)ccn2)CCC1] |
| 483 | 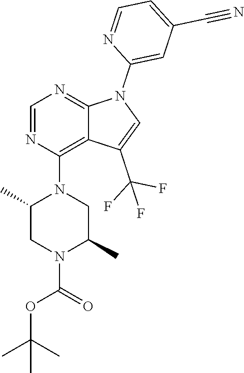 [SMILES: C[C@@H]1CN(c2ncnc3c2c(C(F)(F)F)cn3-c2cc(C#N)ccn2)[C@@H](C)CN1C(=O)OC(C)(C)C] |
| 484 | 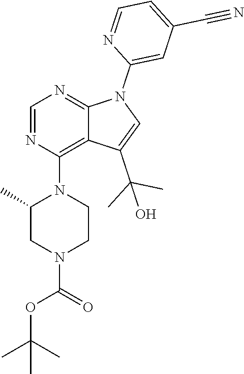 [SMILES: C[C@H]1CN(C(=O)OC(C)(C)C)CCN1c1ncnc2c1c(C(C)(C)O)cn2-c1cc(C#N)ccn1] |
| 485 | 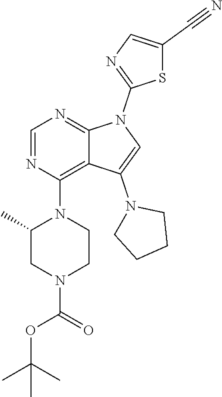 [SMILES: C[C@H]1CN(C(=O)OC(C)(C)C)CCN1c1ncnc2c1c(N1CCCC1)cn2-c1ncc(C#N)s1] |
| 486 | 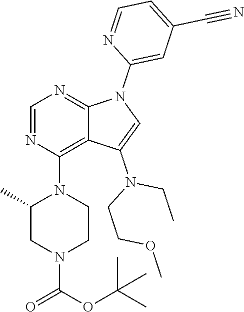 [SMILES: CCN(CCOC)c1cn(-c2cc(C#N)ccn2)c2ncnc(N3CCN(C(=O)OC(C)(C)C)C[C@@H]3C)c12] |
| 487 | 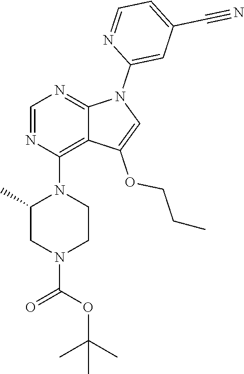 [SMILES: CCCOc1cn(-c2cc(C#N)ccn2)c2ncnc(N3CCN(C(=O)OC(C)(C)C)C[C@@H]3C)c12] |
| 488 | 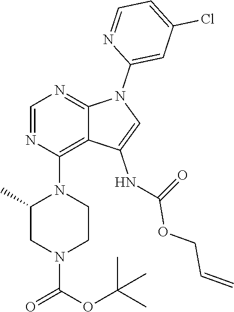 [SMILES: C=CCOC(=O)Nc1cn(-c2cc(Cl)ccn2)c2ncnc(N3CCN(C(=O)OC(C)(C)C)C[C@@H]3C)c12] |
| 489 | 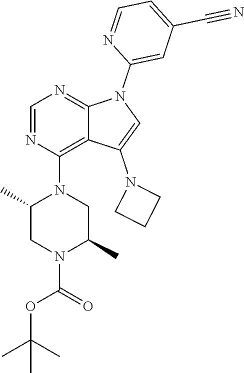 [SMILES: C[C@@H]1CN(c2ncnc3c2c(N2CCC2)cn3-c2cc(C#N)ccn2)[C@@H](C)CN1C(=O)OC(C)(C)C] |
| 490 | 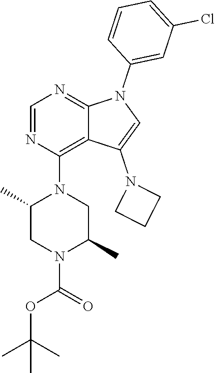 [SMILES: C[C@@H]1CN(c2ncnc3c2c(N2CCC2)cn3-c2cccc(Cl)c2)[C@@H](C)CN1C(=O)OC(C)(C)C] |
| 491 | 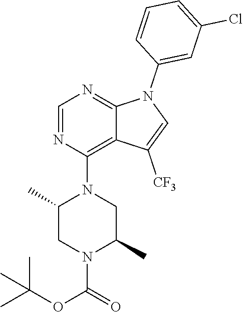 [SMILES: C[C@@H]1CN(c2ncnc3c2c(C(F)(F)F)cn3-c2cccc(Cl)c2)[C@@H](C)CN1C(=O)OC(C)(C)C] |
| 492 | 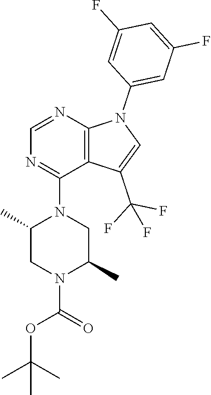 [SMILES: C[C@@H]1CN(c2ncnc3c2c(C(F)(F)F)cn3-c2cc(F)cc(F)c2)[C@@H](C)CN1C(=O)OC(C)(C)C] |
| 493 | 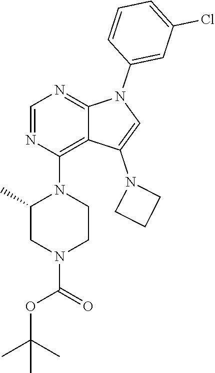 [SMILES: C[C@H]1CN(C(=O)OC(C)(C)C)CCN1c1ncnc2c1c(N1CCC1)cn2-c1cccc(Cl)c1] |
| 494 | 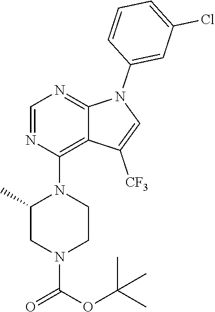 [SMILES: C[C@H]1CN(C(=O)OC(C)(C)C)CCN1c1ncnc2c1c(C(F)(F)F)cn2-c1cccc(Cl)c1] |
| 495 | 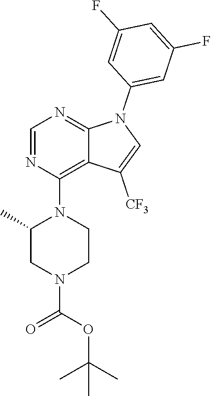 [SMILES: C[C@H]1CN(C(=O)OC(C)(C)C)CCN1c1ncnc2c1c(C(F)(F)F)cn2-c1cc(F)cc(F)c1] |
| 496 | 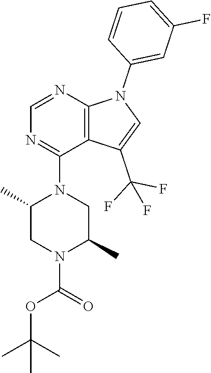 [SMILES: C[C@@H]1CN(c2ncnc3c2c(C(F)(F)F)cn3-c2cccc(F)c2)[C@@H](C)CN1C(=O)OC(C)(C)C] |
| 497 | 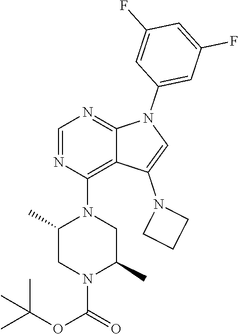 [SMILES: C[C@@H]1CN(c2ncnc3c2c(N2CCC2)cn3-c2cc(F)cc(F)c2)[C@@H](C)CN1C(=O)OC(C)(C)C] |
| 498 | 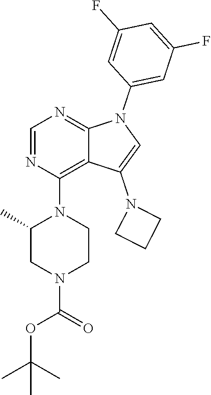 [SMILES: C[C@H]1CN(C(=O)OC(C)(C)C)CCN1c1ncnc2c1c(N1CCC1)cn2-c1cc(F)cc(F)c1] |
| 499 | 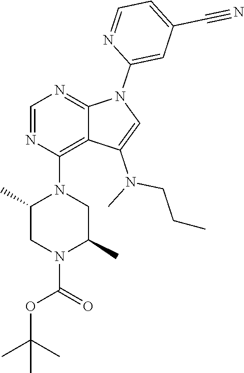 [SMILES: CCCN(C)c1cn(-c2cc(C#N)ccn2)c2ncnc(N3C[C@@H](C)N(C(=O)OC(C)(C)C)C[C@@H]3C)c12] |
| 500 | 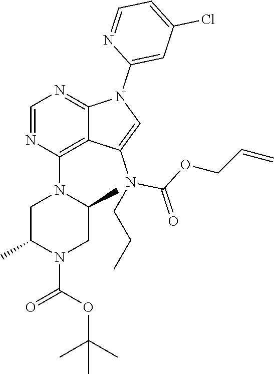 [SMILES: C=CCOC(=O)N(CCC)c1cn(-c2cc(Cl)ccn2)c2ncnc(N3C[C@@H](C)N(C(=O)OC(C)(C)C)C[C@@H]3C)c12] |
| 501 | 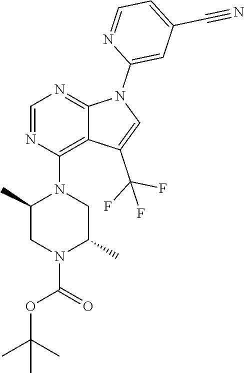 [SMILES: C[C@@H]1CN(C(=O)OC(C)(C)C)[C@@H](C)CN1c1ncnc2c1c(C(F)(F)F)cn2-c1cc(C#N)ccn1] |
| 502 | 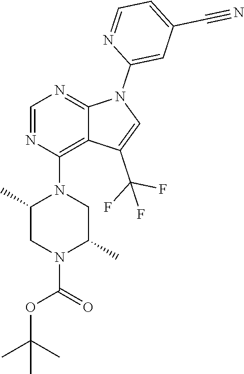 [SMILES: C[C@H]1CN(c2ncnc3c2c(C(F)(F)F)cn3-c2cc(C#N)ccn2)[C@@H](C)CN1C(=O)OC(C)(C)C] |
| 503 | 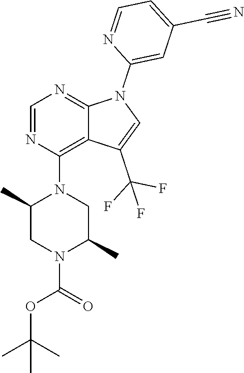 [SMILES: C[C@@H]1CN(c2ncnc3c2c(C(F)(F)F)cn3-c2cc(C#N)ccn2)[C@H](C)CN1C(=O)OC(C)(C)C] |
| 504 | 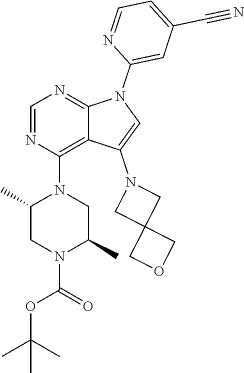 [SMILES: C[C@@H]1CN(c2ncnc3c2c(N2CC4(COC4)C2)cn3-c2cc(C#N)ccn2)[C@@H](C)CN1C(=O)OC(C)(C)C] |
| 505 | 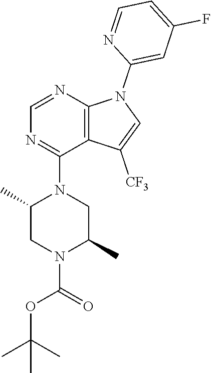 [SMILES: C[C@@H]1CN(c2ncnc3c2c(C(F)(F)F)cn3-c2cc(F)ccn2)[C@@H](C)CN1C(=O)OC(C)(C)C] |
| 506 | 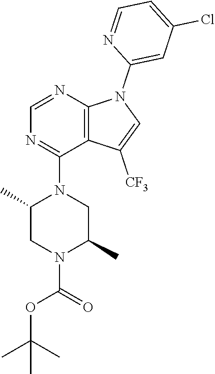 [SMILES: C[C@@H]1CN(c2ncnc3c2c(C(F)(F)F)cn3-c2cc(Cl)ccn2)[C@@H](C)CN1C(=O)OC(C)(C)C] |
| 507 | 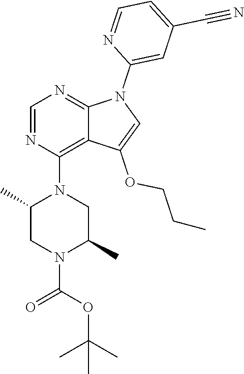 [SMILES: CCCOc1cn(-c2cc(C#N)ccn2)c2ncnc(N3C[C@@H](C)N(C(=O)OC(C)(C)C)C[C@@H]3C)c12] |
| 508 | 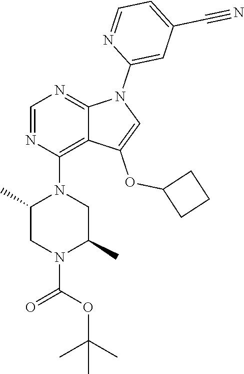 [SMILES: C[C@@H]1CN(c2ncnc3c2c(OC2CCC2)cn3-c2cc(C#N)ccn2)[C@@H](C)CN1C(=O)OC(C)(C)C] |
| 509 | 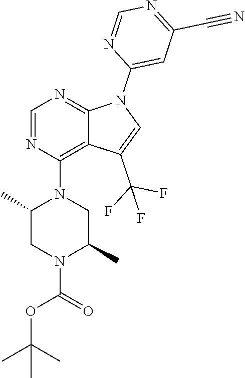 [SMILES: C[C@@H]1CN(c2ncnc3c2c(C(F)(F)F)cn3-c2cc(C#N)ncn2)[C@@H](C)CN1C(=O)OC(C)(C)C] |
| 510 | 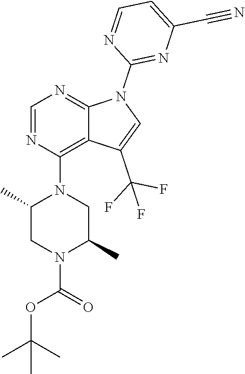 [SMILES: C[C@@H]1CN(c2ncnc3c2c(C(F)(F)F)cn3-c2nccc(C#N)n2)[C@@H](C)CN1C(=O)OC(C)(C)C] |
| 511 | 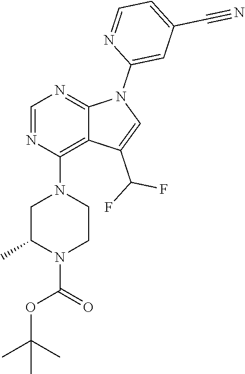 [SMILES: C[C@@H]1CN(c2ncnc3c2c(C(F)F)cn3-c2cc(C#N)ccn2)CCN1C(=O)OC(C)(C)C] |
| 512 | 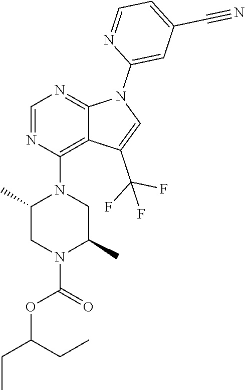 [SMILES: CCC(CC)OC(=O)N1C[C@H](C)N(c2ncnc3c2c(C(F)(F)F)cn3-c2cc(C#N)ccn2)C[C@H]1C] |
| 513 | 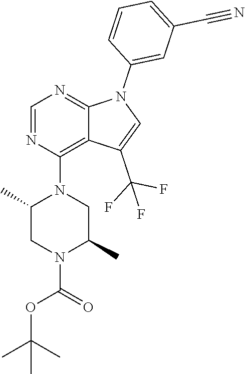 [SMILES: C[C@@H]1CN(c2ncnc3c2c(C(F)(F)F)cn3-c2cccc(C#N)c2)[C@@H](C)CN1C(=O)OC(C)(C)C] |
| 514 | 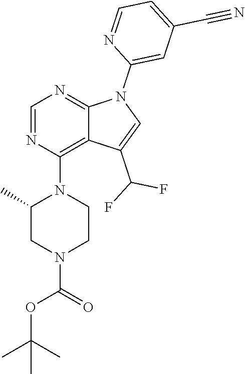 [SMILES: C[C@H]1CN(C(=O)OC(C)(C)C)CCN1c1ncnc2c1c(C(F)F)cn2-c1cc(C#N)ccn1] |
| 515 | 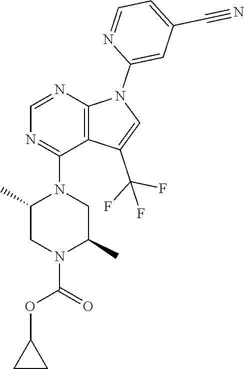 [SMILES: C[C@@H]1CN(c2ncnc3c2c(C(F)(F)F)cn3-c2cc(C#N)ccn2)[C@@H](C)CN1C(=O)OC1CC1] |
| 516 | 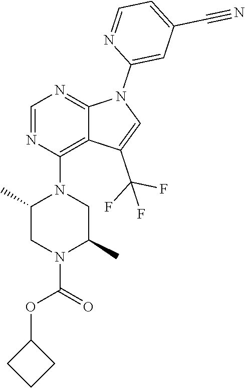 [SMILES: C[C@@H]1CN(c2ncnc3c2c(C(F)(F)F)cn3-c2cc(C#N)ccn2)[C@@H](C)CN1C(=O)OC1CCC1] |
| 517 | 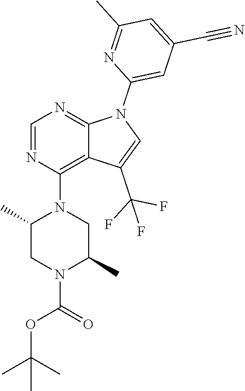 [SMILES: Cc1cc(C#N)cc(-n2cc(C(F)(F)F)c3c(N4C[C@@H](C)N(C(=O)OC(C)(C)C)C[C@@H]4C)ncnc32)n1] |
| 518 | 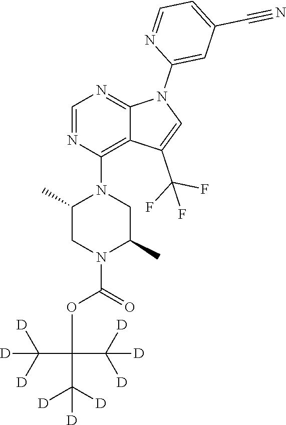 [SMILES: [2H]C([2H])([2H])C(OC(=O)N1C[C@H](C)N(c2ncnc3c2c(C(F)(F)F)cn3-c2cc(C#N)ccn2)C[C@H]1C)(C([2H])([2H])[2H])C([2H])([2H])[2H]] |
| 519 | 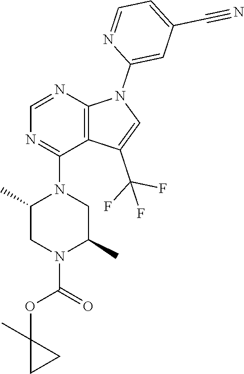 [SMILES: C[C@@H]1CN(c2ncnc3c2c(C(F)(F)F)cn3-c2cc(C#N)ccn2)[C@@H](C)CN1C(=O)OC1(C)CC1] |
| 520 | 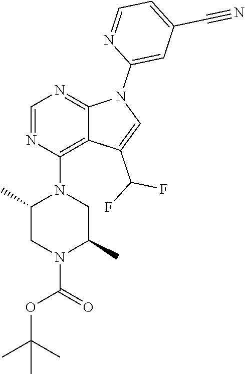 [SMILES: C[C@@H]1CN(c2ncnc3c2c(C(F)F)cn3-c2cc(C#N)ccn2)[C@@H](C)CN1C(=O)OC(C)(C)C] |
| 521 | 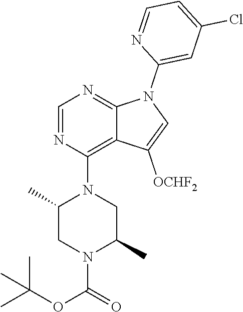 [SMILES: C[C@@H]1CN(c2ncnc3c2c(OC(F)F)cn3-c2cc(Cl)ccn2)[C@@H](C)CN1C(=O)OC(C)(C)C] |
| 522 | 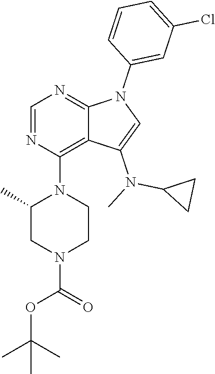 [SMILES: C[C@H]1CN(C(=O)OC(C)(C)C)CCN1c1ncnc2c1c(N(C)C1CC1)cn2-c1cccc(Cl)c1] |
| 523 | 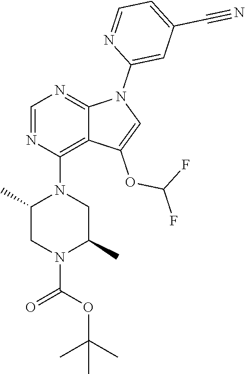 [SMILES: C[C@@H]1CN(c2ncnc3c2c(OC(F)F)cn3-c2cc(C#N)ccn2)[C@@H](C)CN1C(=O)OC(C)(C)C] |
| 524 | 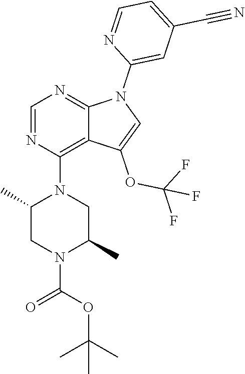 [SMILES: C[C@@H]1CN(c2ncnc3c2c(OC(F)(F)F)cn3-c2cc(C#N)ccn2)[C@@H](C)CN1C(=O)OC(C)(C)C] |
| 525 | 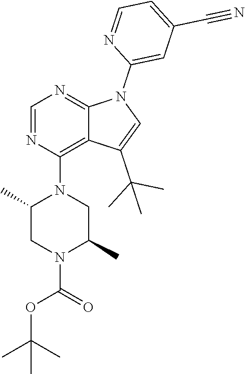 [SMILES: C[C@@H]1CN(c2ncnc3c2c(C(C)(C)C)cn3-c2cc(C#N)ccn2)[C@@H](C)CN1C(=O)OC(C)(C)C] |
| 526 | 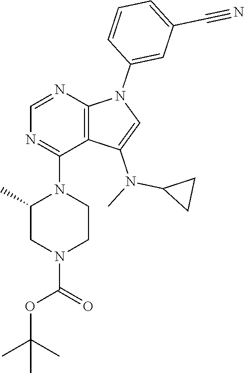 [SMILES: C[C@H]1CN(C(=O)OC(C)(C)C)CCN1c1ncnc2c1c(N(C)C1CC1)cn2-c1cccc(C#N)c1] |
| 527 | 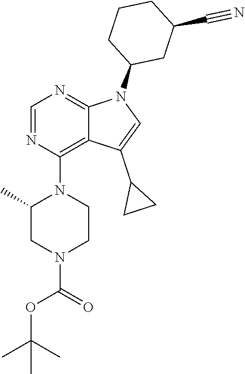 [SMILES: C[C@H]1CN(C(=O)OC(C)(C)C)CCN1c1ncnc2c1c(C1CC1)cn2[C@H]1CCC[C@@H](C#N)C1] |
| 528 | 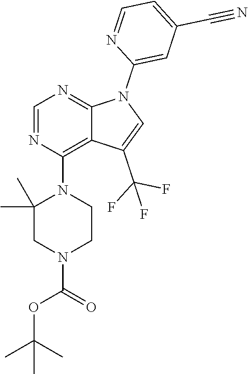 [SMILES: CC(C)(C)OC(=O)N1CCN(c2ncnc3c2c(C(F)(F)F)cn3-c2cc(C#N)ccn2)C(C)(C)C1] |
| 529 | 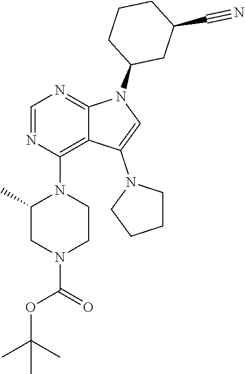 [SMILES: C[C@H]1CN(C(=O)OC(C)(C)C)CCN1c1ncnc2c1c(N1CCCC1)cn2[C@H]1CCC[C@@H](C#N)C1] |
| 530 | 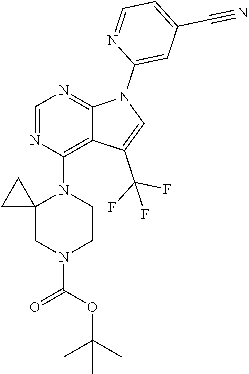 [SMILES: CC(C)(C)OC(=O)N1CCN(c2ncnc3c2c(C(F)(F)F)cn3-c2cc(C#N)ccn2)C2(CC2)C1] |
| 531 | 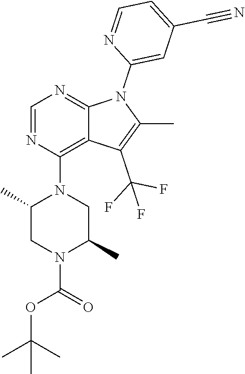 [SMILES: Cc1c(C(F)(F)F)c2c(N3C[C@@H](C)N(C(=O)OC(C)(C)C)C[C@@H]3C)ncnc2n1-c1cc(C#N)ccn1] |
| 532 | 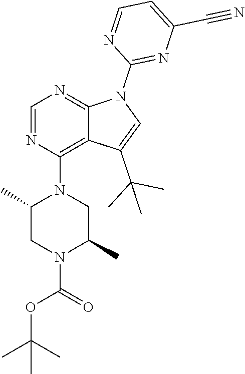 [SMILES: C[C@@H]1CN(c2ncnc3c2c(C(C)(C)C)cn3-c2nccc(C#N)n2)[C@@H](C)CN1C(=O)OC(C)(C)C] |
| 533 | 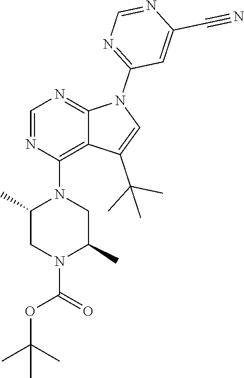 [SMILES: C[C@@H]1CN(c2ncnc3c2c(C(C)(C)C)cn3-c2cc(C#N)ncn2)[C@@H](C)CN1C(=O)OC(C)(C)C] |
| 534 | 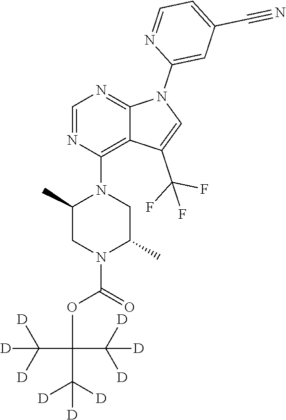 [SMILES: [2H]C([2H])([2H])C(OC(=O)N1C[C@@H](C)N(c2ncnc3c2c(C(F)(F)F)cn3-c2cc(C#N)ccn2)C[C@@H]1C)(C([2H])([2H])[2H])C([2H])([2H])[2H]] |
| 535 | 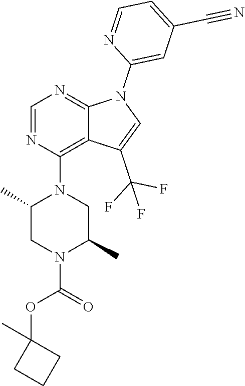 [SMILES: C[C@@H]1CN(c2ncnc3c2c(C(F)(F)F)cn3-c2cc(C#N)ccn2)[C@@H](C)CN1C(=O)OC1(C)CCC1] |
| 536 | 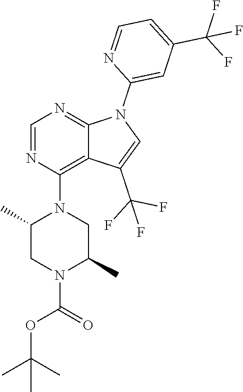 [SMILES: C[C@@H]1CN(c2ncnc3c2c(C(F)(F)F)cn3-c2cc(C(F)(F)F)ccn2)[C@@H](C)CN1C(=O)OC(C)(C)C] |
| 537 | 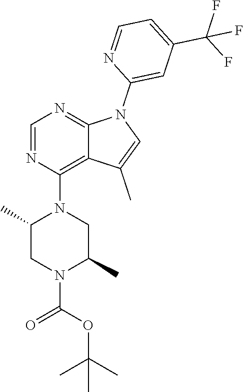 [SMILES: Cc1cn(-c2cc(C(F)(F)F)ccn2)c2ncnc(N3C[C@@H](C)N(C(=O)OC(C)(C)C)C[C@@H]3C)c12] |
| 538 | 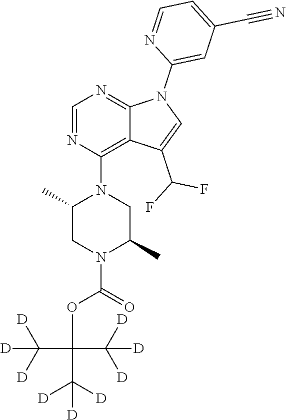 |
| 601 | 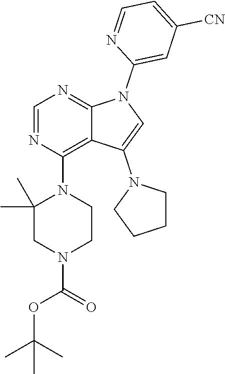 [SMILES: CC(C)(C)OC(=O)N1CCN(c2ncnc3c2c(N2CCCC2)cn3-c2cc(C#N)ccn2)C(C)(C)C1] |
| 602 | 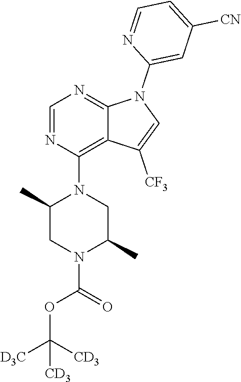 [SMILES: [2H]C([2H])([2H])C(OC(=O)N1C[C@@H](C)N(c2ncnc3c2c(C(F)(F)F)cn3-c2cc(C#N)ccn2)C[C@H]1C)(C([2H])([2H])[2H])C([2H])([2H])[2H]] |
| 603 | 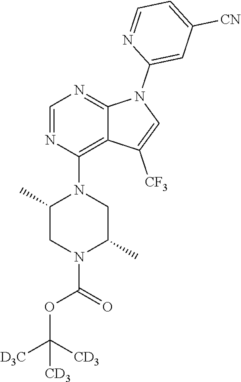 [SMILES: [2H]C([2H])([2H])C(OC(=O)N1C[C@H](C)N(c2ncnc3c2c(C(F)(F)F)cn3-c2cc(C#N)ccn2)C[C@@H]1C)(C([2H])([2H])[2H])C([2H])([2H])[2H]] |
| 604 | 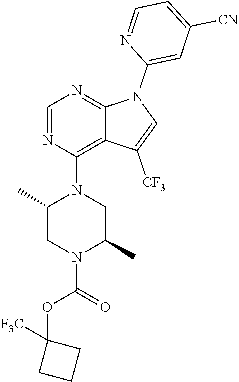 [SMILES: C[C@@H]1CN(c2ncnc3c2c(C(F)(F)F)cn3-c2cc(C#N)ccn2)[C@@H](C)CN1C(=O)OC1(C(F)(F)F)CCC1] |
| 605 | 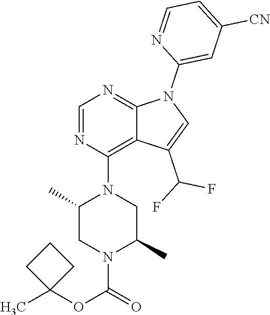 [SMILES: C[C@@H]1CN(c2ncnc3c2c(C(F)F)cn3-c2cc(C#N)ccn2)[C@@H](C)CN1C(=O)OC1(C)CCC1] |
| 606 | 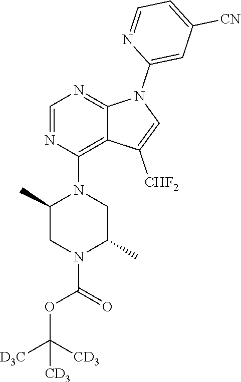 [SMILES: [2H]C([2H])([2H])C(OC(=O)N1C[C@@H](C)N(c2ncnc3c2c(C(F)F)cn3-c2cc(C#N)ccn2)C[C@@H]1C)(C([2H])([2H])[2H])C([2H])([2H])[2H]] |
| 607 | 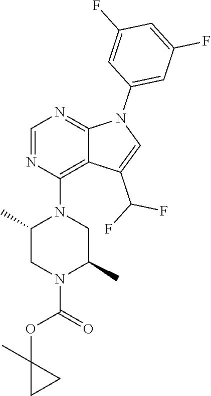 [SMILES: C[C@@H]1CN(c2ncnc3c2c(C(F)F)cn3-c2cc(F)cc(F)c2)[C@@H](C)CN1C(=O)OC1(C)CC1] |
| 608 | 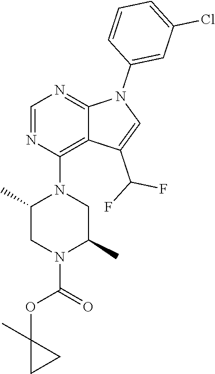 [SMILES: C[C@@H]1CN(c2ncnc3c2c(C(F)F)cn3-c2cccc(Cl)c2)[C@@H](C)CN1C(=O)OC1(C)CC1] |
| 609 | 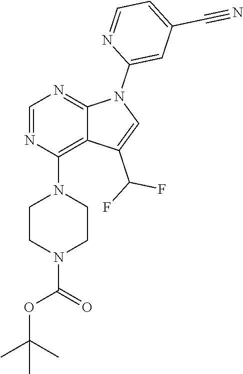 [SMILES: CC(C)(C)OC(=O)N1CCN(c2ncnc3c2c(C(F)F)cn3-c2cc(C#N)ccn2)CC1] |
| 610 | 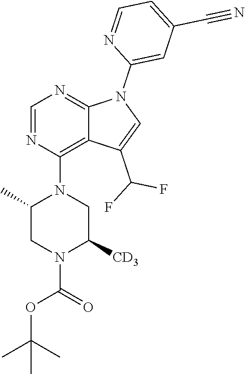 [SMILES: [2H]C([2H])([2H])[C@@H]1CN(c2ncnc3c2c(C(F)F)cn3-c2cc(C#N)ccn2)[C@@H](C)CN1C(=O)OC(C)(C)C] |
| 611 | 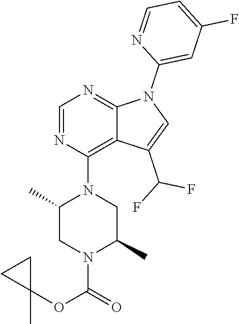 [SMILES: C[C@@H]1CN(c2ncnc3c2c(C(F)F)cn3-c2cc(F)ccn2)[C@@H](C)CN1C(=O)OC1(C)CC1] |
| 612 | 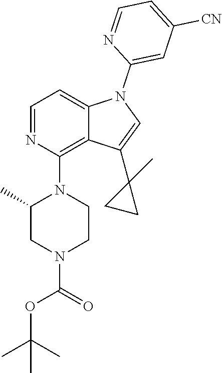 [SMILES: C[C@H]1CN(C(=O)OC(C)(C)C)CCN1c1nccc2c1c(C1(C)CC1)cn2-c1cc(C#N)ccn1] |
| 613 | 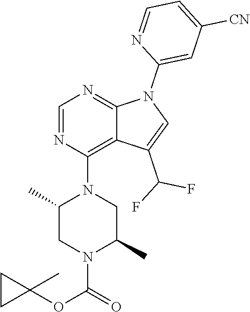 [SMILES: C[C@@H]1CN(c2ncnc3c2c(C(F)F)cn3-c2cc(C#N)ccn2)[C@@H](C)CN1C(=O)OC1(C)CC1] |
| 614 | 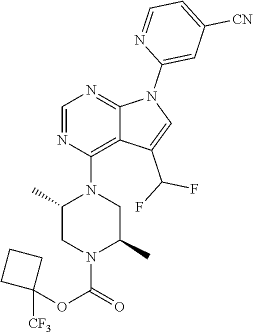 [SMILES: C[C@@H]1CN(c2ncnc3c2c(C(F)F)cn3-c2cc(C#N)ccn2)[C@@H](C)CN1C(=O)OC1(C(F)(F)F)CCC1] |
| 615 | 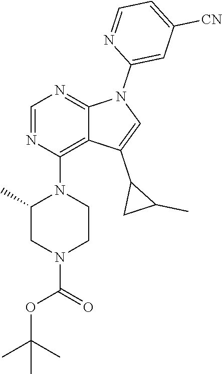 [SMILES: CC1CC1c1cn(-c2cc(C#N)ccn2)c2ncnc(N3CCN(C(=O)OC(C)(C)C)C[C@@H]3C)c12] |
| 616 | 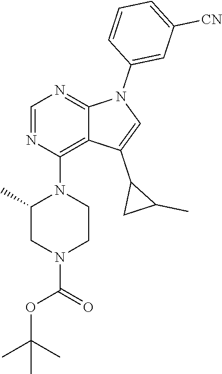 [SMILES: CC1CC1c1cn(-c2cccc(C#N)c2)c2ncnc(N3CCN(C(=O)OC(C)(C)C)C[C@@H]3C)c12] |
| 617 | 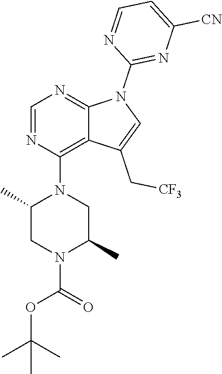 [SMILES: C[C@@H]1CN(c2ncnc3c2c(CC(F)(F)F)cn3-c2nccc(C#N)n2)[C@@H](C)CN1C(=O)OC(C)(C)C] |
| 618 | 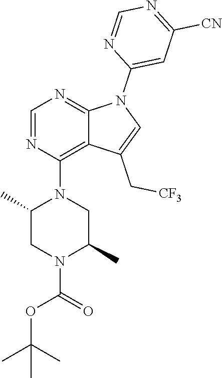 [SMILES: C[C@@H]1CN(c2ncnc3c2c(CC(F)(F)F)cn3-c2cc(C#N)ncn2)[C@@H](C)CN1C(=O)OC(C)(C)C] |
| 619 | 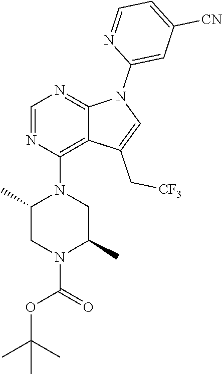 [SMILES: C[C@@H]1CN(c2ncnc3c2c(CC(F)(F)F)cn3-c2cc(C#N)ccn2)[C@@H](C)CN1C(=O)OC(C)(C)C] |
| 620 | 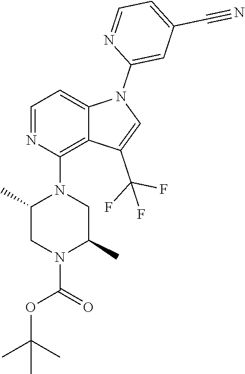 [SMILES: C[C@@H]1CN(c2nccc3c2c(C(F)(F)F)cn3-c2cc(C#N)ccn2)[C@@H](C)CN1C(=O)OC(C)(C)C] |
| 623 | 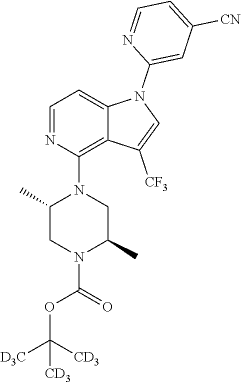 [SMILES: [2H]C([2H])([2H])C(OC(=O)N1C[C@H](C)N(c2nccc3c2c(C(F)(F)F)cn3-c2cc(C#N)ccn2)C[C@H]1C)(C([2H])([2H])[2H])C([2H])([2H])[2H]] |
| 624 | 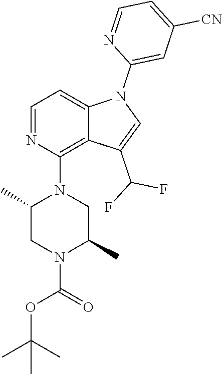 [SMILES: C[C@@H]1CN(c2nccc3c2c(C(F)F)cn3-c2cc(C#N)ccn2)[C@@H](C)CN1C(=O)OC(C)(C)C] |
| 625 | 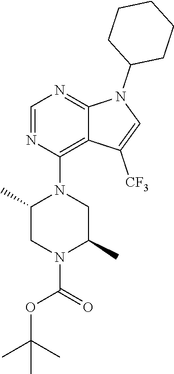 [SMILES: C[C@@H]1CN(c2ncnc3c2c(C(F)(F)F)cn3C2CCCCC2)[C@@H](C)CN1C(=O)OC(C)(C)C] |
| 626 | 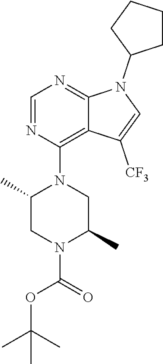 [SMILES: C[C@@H]1CN(c2ncnc3c2c(C(F)(F)F)cn3C2CCCC2)[C@@H](C)CN1C(=O)OC(C)(C)C] |
| 627 | 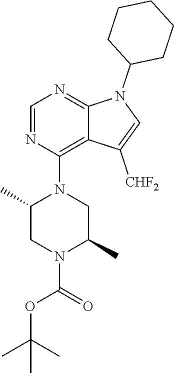 [SMILES: C[C@@H]1CN(c2ncnc3c2c(C(F)F)cn3C2CCCCC2)[C@@H](C)CN1C(=O)OC(C)(C)C] |
| 628 | 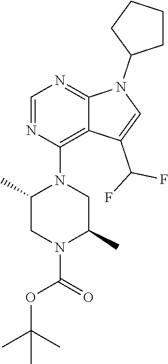 [SMILES: C[C@@H]1CN(c2ncnc3c2c(C(F)F)cn3C2CCCC2)[C@@H](C)CN1C(=O)OC(C)(C)C] |
| 629 | 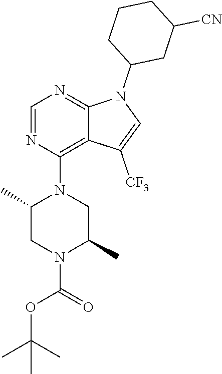 [SMILES: C[C@@H]1CN(c2ncnc3c2c(C(F)(F)F)cn3C2CCCC(C#N)C2)[C@@H](C)CN1C(=O)OC(C)(C)C] |
| 630 | 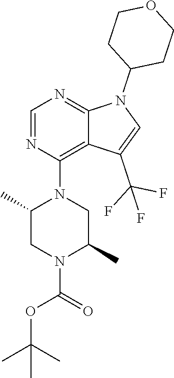 [SMILES: C[C@@H]1CN(c2ncnc3c2c(C(F)(F)F)cn3C2CCOCC2)[C@@H](C)CN1C(=O)OC(C)(C)C] |
| 631 | 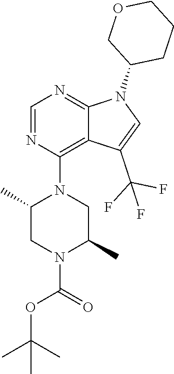 [SMILES: C[C@@H]1CN(c2ncnc3c2c(C(F)(F)F)cn3[C@H]2CCCOC2)[C@@H](C)CN1C(=O)OC(C)(C)C] |
| 632 | 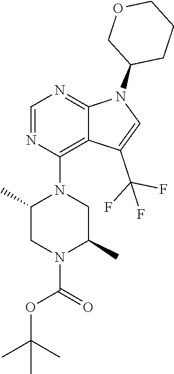 [SMILES: C[C@@H]1CN(c2ncnc3c2c(C(F)(F)F)cn3[C@@H]2CCCOC2)[C@@H](C)CN1C(=O)OC(C)(C)C] |
| 633 | 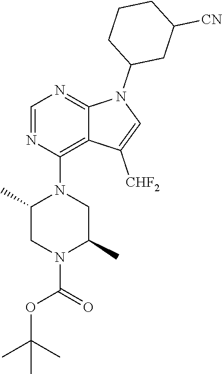 [SMILES: C[C@@H]1CN(c2ncnc3c2c(C(F)F)cn3C2CCCC(C#N)C2)[C@@H](C)CN1C(=O)OC(C)(C)C] |
| 634 | 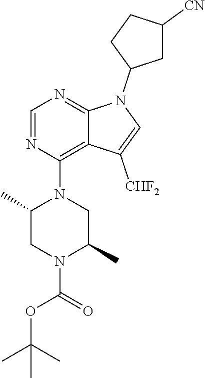 [SMILES: C[C@@H]1CN(c2ncnc3c2c(C(F)F)cn3C2CCC(C#N)C2)[C@@H](C)CN1C(=O)OC(C)(C)C] |
| 635 | 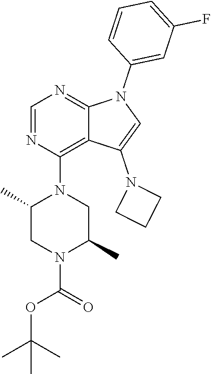 [SMILES: C[C@@H]1CN(c2ncnc3c2c(N2CCC2)cn3-c2cccc(F)c2)[C@@H](C)CN1C(=O)OC(C)(C)C] |
| 636 | 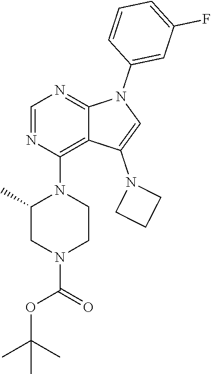 [SMILES: C[C@H]1CN(C(=O)OC(C)(C)C)CCN1c1ncnc2c1c(N1CCC1)cn2-c1cccc(F)c1] |
| 637 | 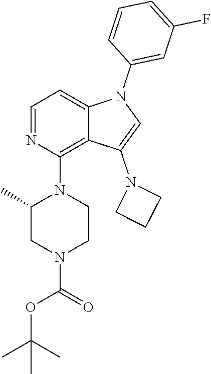 [SMILES: C[C@H]1CN(C(=O)OC(C)(C)C)CCN1c1nccc2c1c(N1CCC1)cn2-c1cccc(F)c1] |
| 638 | 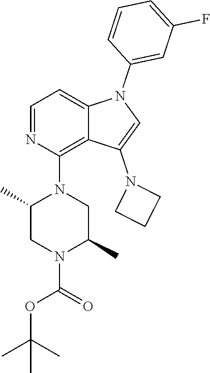 [SMILES: C[C@@H]1CN(c2nccc3c2c(N2CCC2)cn3-c2cccc(F)c2)[C@@H](C)CN1C(=O)OC(C)(C)C] |
| 641 | 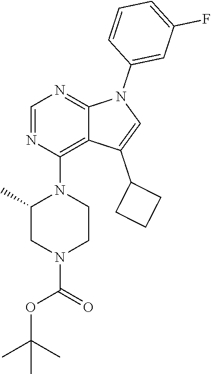 [SMILES: C[C@H]1CN(C(=O)OC(C)(C)C)CCN1c1ncnc2c1c(C1CCC1)cn2-c1cccc(F)c1] |
| 642 | 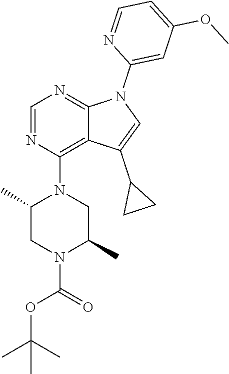 |
| 643 | 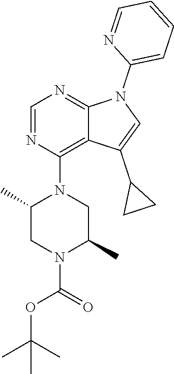 [SMILES: C[C@@H]1CN(c2ncnc3c2c(C2CC2)cn3-c2ccccn2)[C@@H](C)CN1C(=O)OC(C)(C)C] |
| 644 | 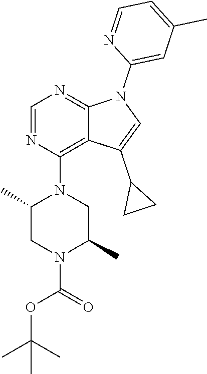 |
| 645 | 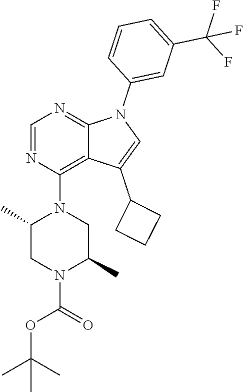 [SMILES: C[C@@H]1CN(c2ncnc3c2c(C2CCC2)cn3-c2cccc(C(F)(F)F)c2)[C@@H](C)CN1C(=O)OC(C)(C)C] |
| 646 | 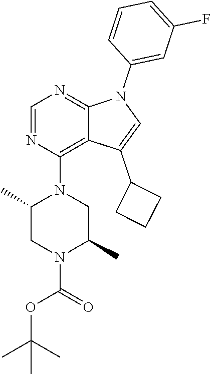 [SMILES: C[C@@H]1CN(c2ncnc3c2c(C2CCC2)cn3-c2cccc(F)c2)[C@@H](C)CN1C(=O)OC(C)(C)C] |
| 647 | 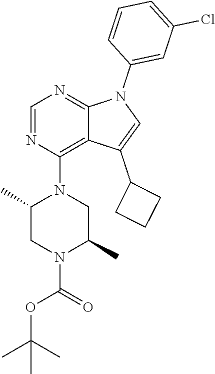 [SMILES: C[C@@H]1CN(c2ncnc3c2c(C2CCC2)cn3-c2cccc(Cl)c2)[C@@H](C)CN1C(=O)OC(C)(C)C] |
| 648 | 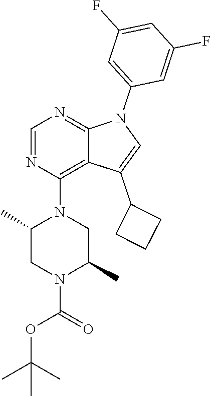 [SMILES: C[C@@H]1CN(c2ncnc3c2c(C2CCC2)cn3-c2cc(F)cc(F)c2)[C@@H](C)CN1C(=O)OC(C)(C)C] |
| 649 | 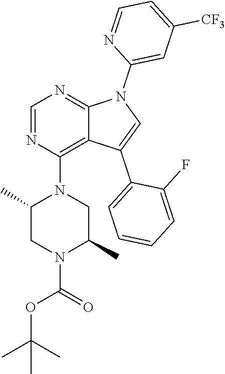 [SMILES: C[C@@H]1CN(c2ncnc3c2c(-c2ccccc2F)cn3-c2cc(C(F)(F)F)ccn2)[C@@H](C)CN1C(=O)OC(C)(C)C] |
| 650 | 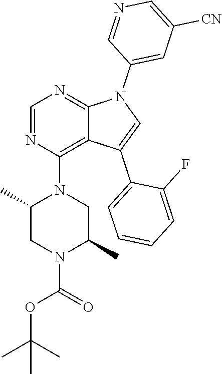 [SMILES: C[C@@H]1CN(c2ncnc3c2c(-c2ccccc2F)cn3-c2cncc(C#N)c2)[C@@H](C)CN1C(=O)OC(C)(C)C] |
| 651 | 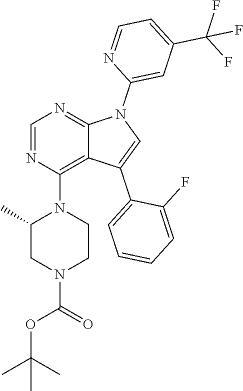 [SMILES: C[C@H]1CN(C(=O)OC(C)(C)C)CCN1c1ncnc2c1c(-c1ccccc1F)cn2-c1cc(C(F)(F)F)ccn1] |
| 652 | 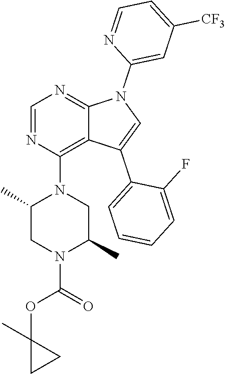 [SMILES: C[C@@H]1CN(c2ncnc3c2c(-c2ccccc2F)cn3-c2cc(C(F)(F)F)ccn2)[C@@H](C)CN1C(=O)OC1(C)CC1] |
| 653 | 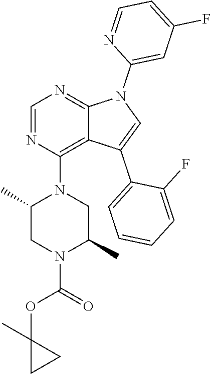 [SMILES: C[C@@H]1CN(c2ncnc3c2c(-c2ccccc2F)cn3-c2cc(F)ccn2)[C@@H](C)CN1C(=O)OC1(C)CC1] |
| 654 | 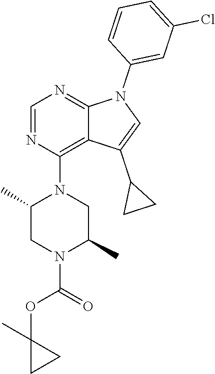 [SMILES: C[C@@H]1CN(c2ncnc3c2c(C2CC2)cn3-c2cccc(Cl)c2)[C@@H](C)CN1C(=O)OC1(C)CC1] |
| 655 | 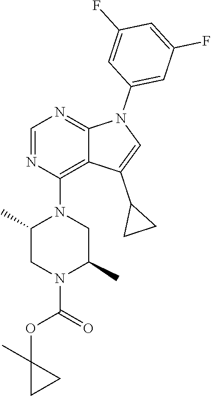 [SMILES: C[C@@H]1CN(c2ncnc3c2c(C2CC2)cn3-c2cc(F)cc(F)c2)[C@@H](C)CN1C(=O)OC1(C)CC1] |
| 656 | 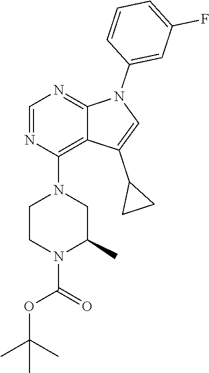 [SMILES: C[C@@H]1CN(c2ncnc3c2c(C2CC2)cn3-c2cccc(F)c2)CCN1C(=O)OC(C)(C)C] |
| 657 | 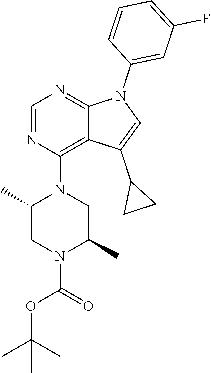 [SMILES: C[C@@H]1CN(c2ncnc3c2c(C2CC2)cn3-c2cccc(F)c2)[C@@H](C)CN1C(=O)OC(C)(C)C] |
| 658 | 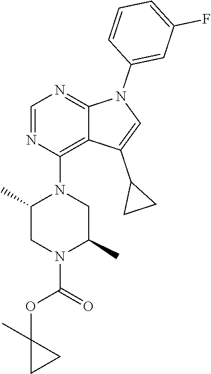 [SMILES: C[C@@H]1CN(c2ncnc3c2c(C2CC2)cn3-c2cccc(F)c2)[C@@H](C)CN1C(=O)OC1(C)CC1] |
| 659 | 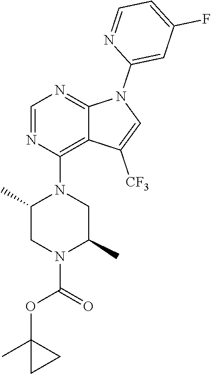 [SMILES: C[C@@H]1CN(c2ncnc3c2c(C(F)(F)F)cn3-c2cc(F)ccn2)[C@@H](C)CN1C(=O)OC1(C)CC1] |
| 660 | 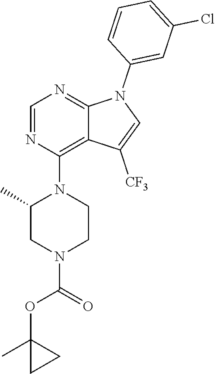 [SMILES: C[C@H]1CN(C(=O)OC2(C)CC2)CCN1c1ncnc2c1c(C(F)(F)F)cn2-c1cccc(Cl)c1] |
| 661 | 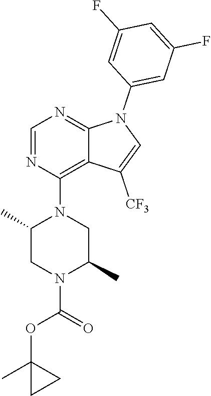 [SMILES: C[C@@H]1CN(c2ncnc3c2c(C(F)(F)F)cn3-c2cc(F)cc(F)c2)[C@@H](C)CN1C(=O)OC1(C)CC1] |
| 662 | 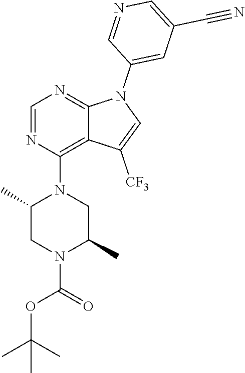 [SMILES: C[C@@H]1CN(c2ncnc3c2c(C(F)(F)F)cn3-c2cncc(C#N)c2)[C@@H](C)CN1C(=O)OC(C)(C)C] |
| 663 | 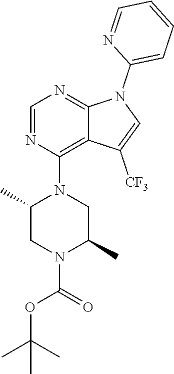 [SMILES: C[C@@H]1CN(c2ncnc3c2c(C(F)(F)F)cn3-c2ccccn2)[C@@H](C)CN1C(=O)OC(C)(C)C] |
| 664 | 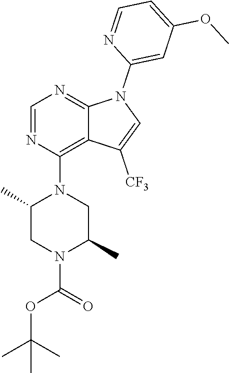 [SMILES: COc1ccnc(-n2cc(C(F)(F)F)c3c(N4C[C@@H](C)N(C(=O)OC(C)(C)C)C[C@@H]4C)ncnc32)c1] |
| 665 | 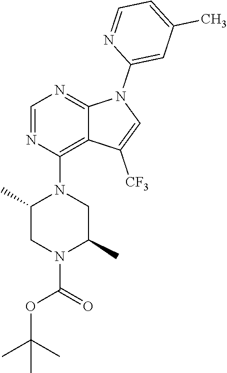 [SMILES: Cc1ccnc(-n2cc(C(F)(F)F)c3c(N4C[C@@H](C)N(C(=O)OC(C)(C)C)C[C@@H]4C)ncnc32)c1] |
| 666 | 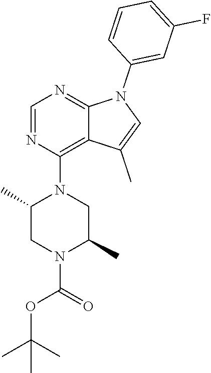 [SMILES: Cc1cn(-c2cccc(F)c2)c2ncnc(N3C[C@@H](C)N(C(=O)OC(C)(C)C)C[C@@H]3C)c12] |
| 667 | 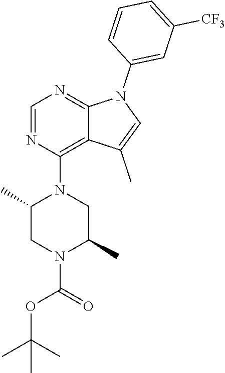 [SMILES: Cc1cn(-c2cccc(C(F)(F)F)c2)c2ncnc(N3C[C@@H](C)N(C(=O)OC(C)(C)C)C[C@@H]3C)c12] |
| 668 | 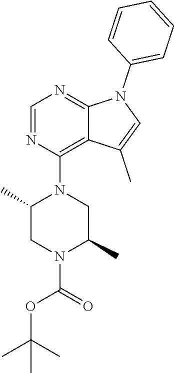 [SMILES: Cc1cn(-c2ccccc2)c2ncnc(N3C[C@@H](C)N(C(=O)OC(C)(C)C)C[C@@H]3C)c12] |
| 669 | 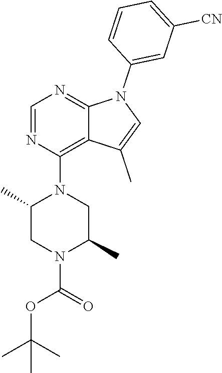 [SMILES: Cc1cn(-c2cccc(C#N)c2)c2ncnc(N3C[C@@H](C)N(C(=O)OC(C)(C)C)C[C@@H]3C)c12] |
| 670 | 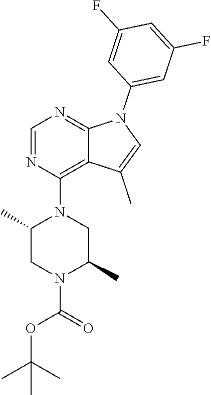 [SMILES: Cc1cn(-c2cc(F)cc(F)c2)c2ncnc(N3C[C@@H](C)N(C(=O)OC(C)(C)C)C[C@@H]3C)c12] |
| 671 | 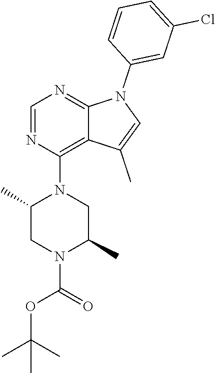 [SMILES: Cc1cn(-c2cccc(Cl)c2)c2ncnc(N3C[C@@H](C)N(C(=O)OC(C)(C)C)C[C@@H]3C)c12] |
| 672 | 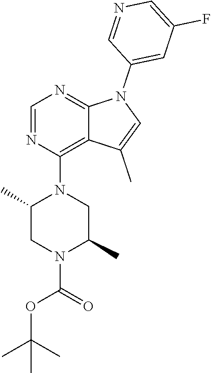 [SMILES: Cc1cn(-c2cncc(F)c2)c2ncnc(N3C[C@@H](C)N(C(=O)OC(C)(C)C)C[C@@H]3C)c12] |
| 673 | 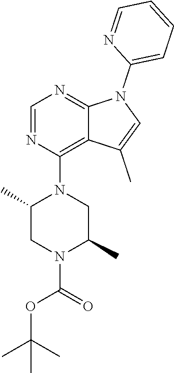 [SMILES: Cc1cn(-c2ccccn2)c2ncnc(N3C[C@@H](C)N(C(=O)OC(C)(C)C)C[C@@H]3C)c12] |
| 674 | 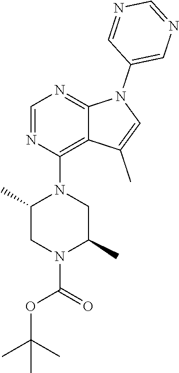 [SMILES: Cc1cn(-c2cncnc2)c2ncnc(N3C[C@@H](C)N(C(=O)OC(C)(C)C)C[C@@H]3C)c12] |
| 675 | 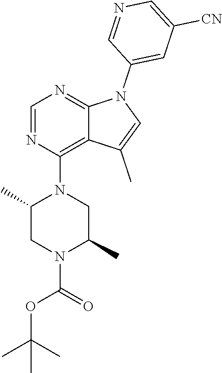 [SMILES: Cc1cn(-c2cncc(C#N)c2)c2ncnc(N3C[C@@H](C)N(C(=O)OC(C)(C)C)C[C@@H]3C)c12] |
| 676 | 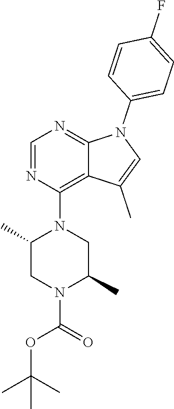 [SMILES: Cc1cn(-c2ccc(F)cc2)c2ncnc(N3C[C@@H](C)N(C(=O)OC(C)(C)C)C[C@@H]3C)c12] |
| 677 | 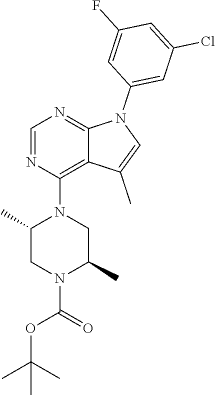 [SMILES: Cc1cn(-c2cc(F)cc(Cl)c2)c2ncnc(N3C[C@@H](C)N(C(=O)OC(C)(C)C)C[C@@H]3C)c12] |
| 678 | 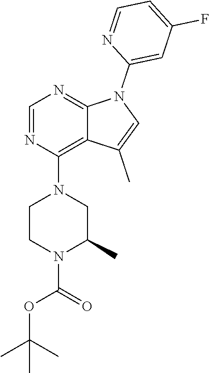 [SMILES: Cc1cn(-c2cc(F)ccn2)c2ncnc(N3CCN(C(=O)OC(C)(C)C)[C@H](C)C3)c12] |
| 679 | 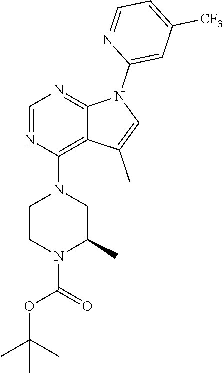 [SMILES: Cc1cn(-c2cc(C(F)(F)F)ccn2)c2ncnc(N3CCN(C(=O)OC(C)(C)C)[C@H](C)C3)c12] |
| 680 | 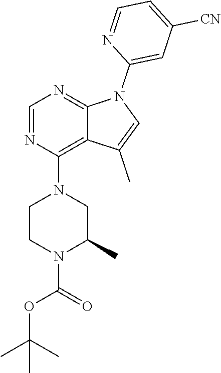 [SMILES: Cc1cn(-c2cc(C#N)ccn2)c2ncnc(N3CCN(C(=O)OC(C)(C)C)[C@H](C)C3)c12] |
| 681 | 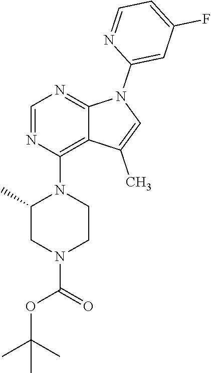 [SMILES: Cc1cn(-c2cc(F)ccn2)c2ncnc(N3CCN(C(=O)OC(C)(C)C)C[C@@H]3C)c12] |
| 682 | 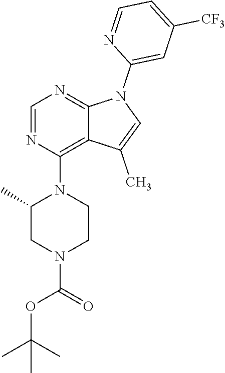 [SMILES: Cc1cn(-c2cc(C(F)(F)F)ccn2)c2ncnc(N3CCN(C(=O)OC(C)(C)C)C[C@@H]3C)c12] |
| 683 | 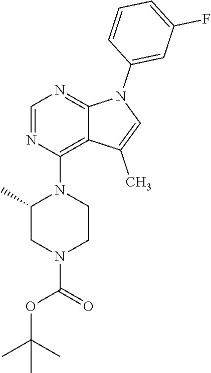 [SMILES: Cc1cn(-c2cccc(F)c2)c2ncnc(N3CCN(C(=O)OC(C)(C)C)C[C@@H]3C)c12] |
| 684 | 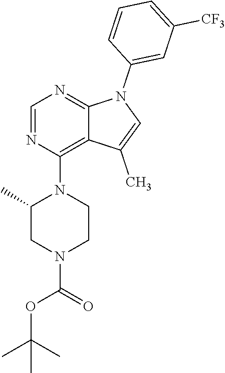 [SMILES: Cc1cn(-c2cccc(C(F)(F)F)c2)c2ncnc(N3CCN(C(=O)OC(C)(C)C)C[C@@H]3C)c12] |
| 685 | 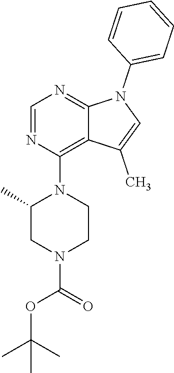 [SMILES: Cc1cn(-c2ccccc2)c2ncnc(N3CCN(C(=O)OC(C)(C)C)C[C@@H]3C)c12] |
| 686 | 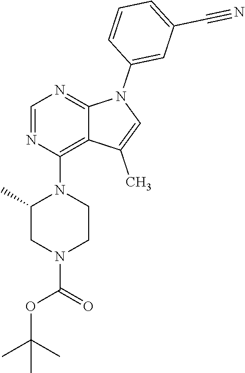 [SMILES: Cc1cn(-c2cccc(C#N)c2)c2ncnc(N3CCN(C(=O)OC(C)(C)C)C[C@@H]3C)c12] |
| 687 | 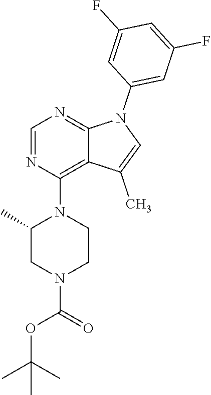 [SMILES: Cc1cn(-c2cc(F)cc(F)c2)c2ncnc(N3CCN(C(=O)OC(C)(C)C)C[C@@H]3C)c12] |
| 688 | 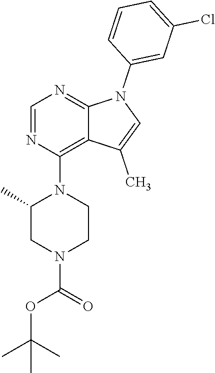 [SMILES: Cc1cn(-c2cccc(Cl)c2)c2ncnc(N3CCN(C(=O)OC(C)(C)C)C[C@@H]3C)c12] |
| 689 | 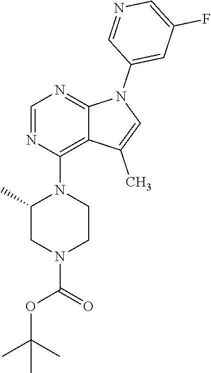 [SMILES: Cc1cn(-c2cncc(F)c2)c2ncnc(N3CCN(C(=O)OC(C)(C)C)C[C@@H]3C)c12] |
| 690 | 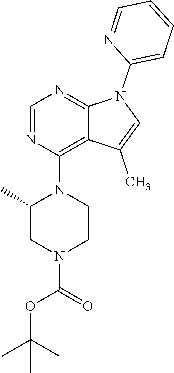 [SMILES: Cc1cn(-c2ccccn2)c2ncnc(N3CCN(C(=O)OC(C)(C)C)C[C@@H]3C)c12] |
| 691 | 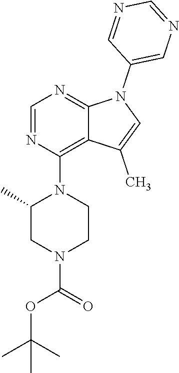 [SMILES: Cc1cn(-c2cncnc2)c2ncnc(N3CCN(C(=O)OC(C)(C)C)C[C@@H]3C)c12] |
| 692 | 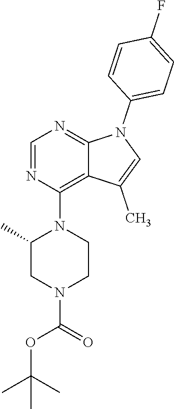 [SMILES: Cc1cn(-c2ccc(F)cc2)c2ncnc(N3CCN(C(=O)OC(C)(C)C)C[C@@H]3C)c12] |
| 693 | 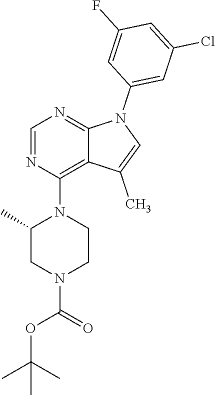 [SMILES: Cc1cn(-c2cc(F)cc(Cl)c2)c2ncnc(N3CCN(C(=O)OC(C)(C)C)C[C@@H]3C)c12] |
| 694 | 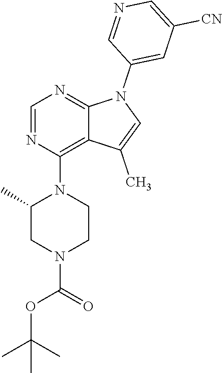 [SMILES: Cc1cn(-c2cncc(C#N)c2)c2ncnc(N3CCN(C(=O)OC(C)(C)C)C[C@@H]3C)c12] |
| 695 | 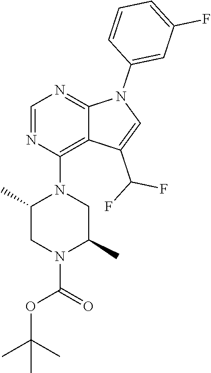 [SMILES: C[C@@H]1CN(c2ncnc3c2c(C(F)F)cn3-c2cccc(F)c2)[C@@H](C)CN1C(=O)OC(C)(C)C] |
| 696 | 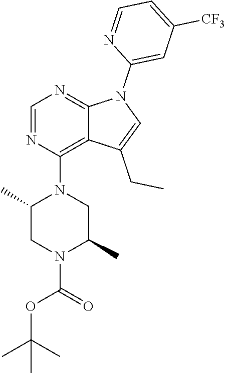 [SMILES: CCc1cn(-c2cc(C(F)(F)F)ccn2)c2ncnc(N3C[C@@H](C)N(C(=O)OC(C)(C)C)C[C@@H]3C)c12] |
| 697 | 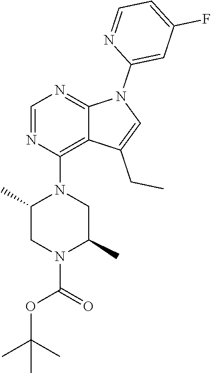 [SMILES: CCc1cn(-c2cc(F)ccn2)c2ncnc(N3C[C@@H](C)N(C(=O)OC(C)(C)C)C[C@@H]3C)c12] |
| 698 | 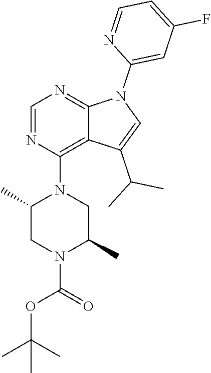 [SMILES: CC(C)c1cn(-c2cc(F)ccn2)c2ncnc(N3C[C@@H](C)N(C(=O)OC(C)(C)C)C[C@@H]3C)c12] |
| 699 | 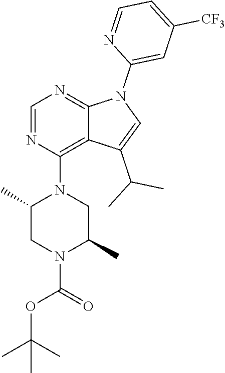 [SMILES: CC(C)c1cn(-c2cc(C(F)(F)F)ccn2)c2ncnc(N3C[C@@H](C)N(C(=O)OC(C)(C)C)C[C@@H]3C)c12] |
| 700 | 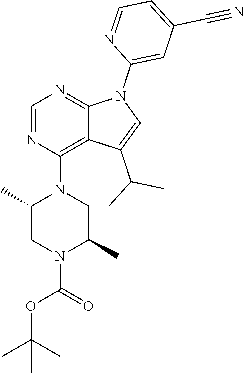 [SMILES: CC(C)c1cn(-c2cc(C#N)ccn2)c2ncnc(N3C[C@@H](C)N(C(=O)OC(C)(C)C)C[C@@H]3C)c12] |
| 701 | 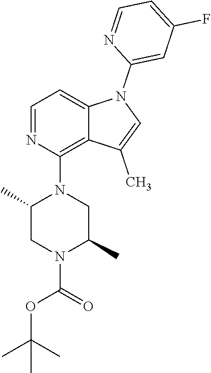 [SMILES: Cc1cn(-c2cc(F)ccn2)c2ccnc(N3C[C@@H](C)N(C(=O)OC(C)(C)C)C[C@@H]3C)c12] |
| 702 | 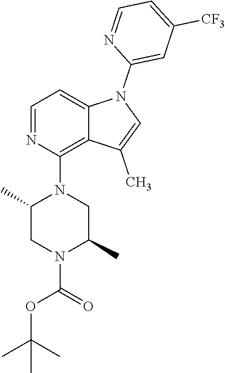 [SMILES: Cc1cn(-c2cc(C(F)(F)F)ccn2)c2ccnc(N3C[C@@H](C)N(C(=O)OC(C)(C)C)C[C@@H]3C)c12] |
| 703 | 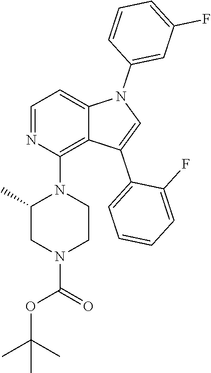 [SMILES: C[C@H]1CN(C(=O)OC(C)(C)C)CCN1c1nccc2c1c(-c1ccccc1F)cn2-c1cccc(F)c1] |
| 704 | 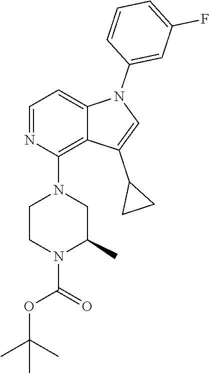 [SMILES: C[C@@H]1CN(c2nccc3c2c(C2CC2)cn3-c2cccc(F)c2)CCN1C(=O)OC(C)(C)C] |
| 705 | 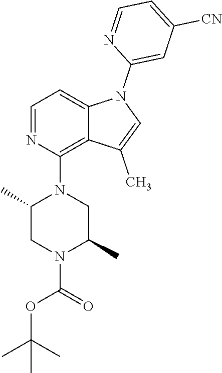 [SMILES: Cc1cn(-c2cc(C#N)ccn2)c2ccnc(N3C[C@@H](C)N(C(=O)OC(C)(C)C)C[C@@H]3C)c12] |
| 706 | 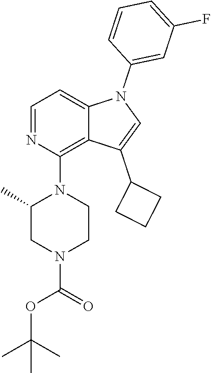 [SMILES: C[C@H]1CN(C(=O)OC(C)(C)C)CCN1c1nccc2c1c(C1CCC1)cn2-c1cccc(F)c1] |
| 707 | 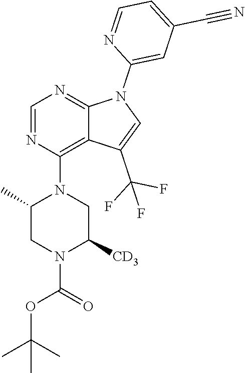 [SMILES: [2H]C([2H])([2H])[C@@H]1CN(c2ncnc3c2c(C(F)(F)F)cn3-c2cc(C#N)ccn2)[C@@H](C)CN1C(=O)OC(C)(C)C] |
| 708 | 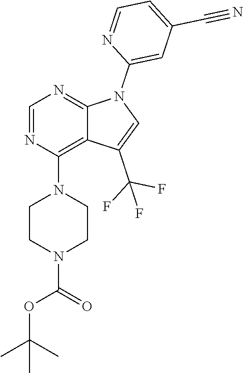 [SMILES: CC(C)(C)OC(=O)N1CCN(c2ncnc3c2c(C(F)(F)F)cn3-c2cc(C#N)ccn2)CC1] |
| 709 | 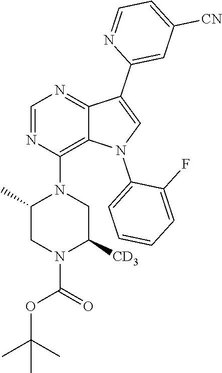 [SMILES: [2H]C([2H])([2H])[C@@H]1CN(c2ncnc3c(-c4cc(C#N)ccn4)cn(-c4ccccc4F)c23)[C@@H](C)CN1C(=O)OC(C)(C)C] |
| 710 | 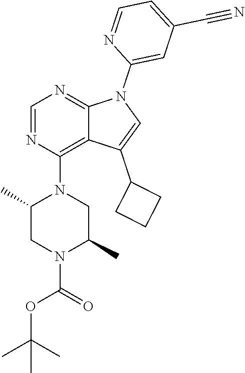 [SMILES: C[C@@H]1CN(c2ncnc3c2c(C2CCC2)cn3-c2cc(C#N)ccn2)[C@@H](C)CN1C(=O)OC(C)(C)C] |
| 711 | 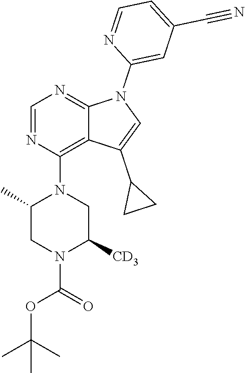 [SMILES: [2H]C([2H])([2H])[C@@H]1CN(c2ncnc3c2c(C2CC2)cn3-c2cc(C#N)ccn2)[C@@H](C)CN1C(=O)OC(C)(C)C] |
| 712 | 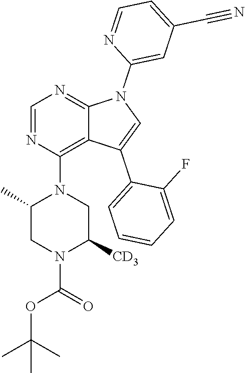 [SMILES: [2H]C([2H])([2H])[C@@H]1CN(c2ncnc3c2c(-c2ccccc2F)cn3-c2cc(C#N)ccn2)[C@@H](C)CN1C(=O)OC(C)(C)C] |
| 713 | 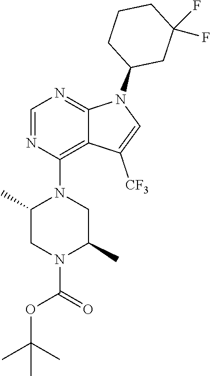 [SMILES: C[C@@H]1CN(c2ncnc3c2c(C(F)(F)F)cn3[C@H]2CCCC(F)(F)C2)[C@@H](C)CN1C(=O)OC(C)(C)C] |
| 714 | 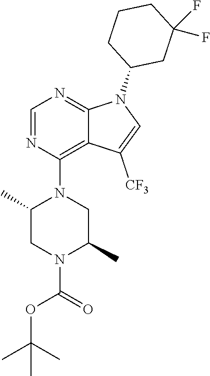 [SMILES: C[C@@H]1CN(c2ncnc3c2c(C(F)(F)F)cn3[C@@H]2CCCC(F)(F)C2)[C@@H](C)CN1C(=O)OC(C)(C)C] |
| 715 | 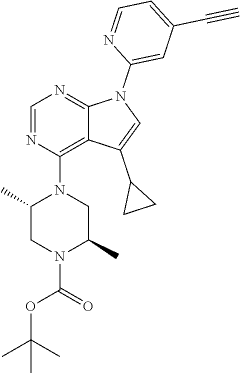 [SMILES: C#Cc1ccnc(-n2cc(C3CC3)c3c(N4C[C@@H](C)N(C(=O)OC(C)(C)C)C[C@@H]4C)ncnc32)c1] |
| 716 | 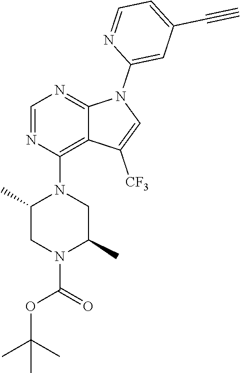 [SMILES: C#Cc1ccnc(-n2cc(C(F)(F)F)c3c(N4C[C@@H](C)N(C(=O)OC(C)(C)C)C[C@@H]4C)ncnc32)c1] |
| 717 | 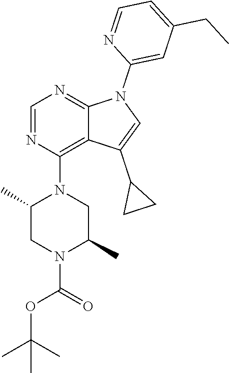 [SMILES: CCc1ccnc(-n2cc(C3CC3)c3c(N4C[C@@H](C)N(C(=O)OC(C)(C)C)C[C@@H]4C)ncnc32)c1] |
| 718 | 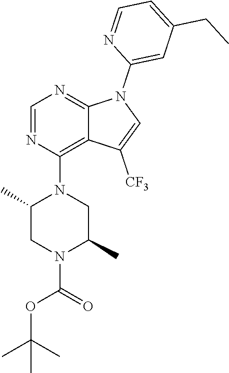 [SMILES: CCc1ccnc(-n2cc(C(F)(F)F)c3c(N4C[C@@H](C)N(C(=O)OC(C)(C)C)C[C@@H]4C)ncnc32)c1] |
| 1001 | 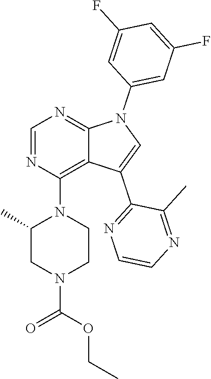 [SMILES: CCOC(=O)N1CCN(c2ncnc3c2c(-c2nccnc2C)cn3-c2cc(F)cc(F)c2)[C@@H](C)C1] |
| 1002 | 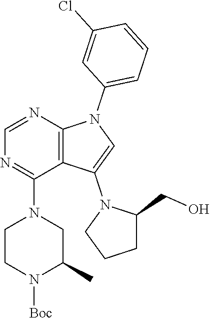 [SMILES: C[C@@H]1CN(c2ncnc3c2c(N2CCC[C@@H]2CO)cn3-c2cccc(Cl)c2)CCN1C(=O)OC(C)(C)C] |
| 1003 | 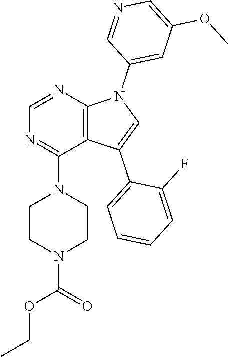 [SMILES: CCOC(=O)N1CCN(c2ncnc3c2c(-c2ccccc2F)cn3-c2cncc(OC)c2)CC1] |
| 1004 | 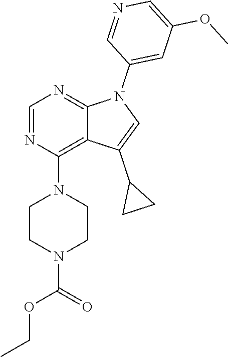 [SMILES: CCOC(=O)N1CCN(c2ncnc3c2c(C2CC2)cn3-c2cncc(OC)c2)CC1] |
| 1005 | 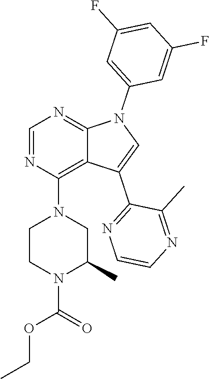 [SMILES: CCOC(=O)N1CCN(c2ncnc3c2c(-c2nccnc2C)cn3-c2cc(F)cc(F)c2)C[C@H]1C] |
| 1006 | 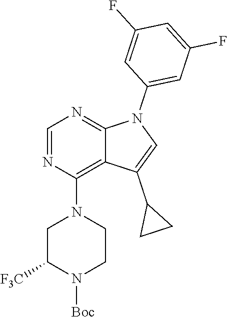 [SMILES: CC(C)(C)OC(=O)N1CCN(c2ncnc3c2c(C2CC2)cn3-c2cc(F)cc(F)c2)C[C@H]1C(F)(F)F] |

### Deuterated Compounds

In some embodiments, compounds described herein herein (e.g., a compound of Formula I, Ia, Ib, or Ic) are deuterium enriched.

Deuterium (D or ²H) is a stable, non-radioactive isotope of hydrogen and has an atomic weight of 2.0144. Hydrogen naturally occurs as a mixture of the isotopes ¹H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015% should be considered unnatural and, as a result, novel over their non-enriched counterparts.

The effects of deuterium modification on a compound's metabolic properties are not predictable, even when deuterium atoms are incorporated at known sites of metabolism. Only by actually preparing and testing a deuterated compound can one determine if and how the rate of metabolism will differ from that of its non-deuterated counterpart. See, for example, Fukuto et al. (J. Med. Chem. 1991, 34, 2871-76). Many compounds have multiple sites where metabolism is possible. The site(s) where deuterium substitution is required and the extent of deuteration necessary to see an effect on metabolism, if any, will be different for each compound.

Unless otherwise stated, when a position is designated specifically as “H” or “hydrogen,” the position is understood to have hydrogen at its natural abundance isotopic composition. Also, unless otherwise stated, when a position is designated specifically as “D” or “deuterium,” the position is understood to have deuterium at an abundance that is at least 3000 times greater than the natural abundance of deuterium, which is 0.015% (i.e., the term “D” or “deuterium” indicates at least 45% incorporation of deuterium).

The term “isotopic enrichment factor” as used herein means the ratio between the isotopic abundance of D at the specified position in a compound of this invention and the naturally occurring abundance of that isotope.

Increasing the amount of deuterium present in a compound herein (e.g., a compound of Formula I, Ia, Ib, or Ic) is called “deuterium-enrichment,” and such compounds are referred to as “deuterium-enriched” compounds. If not specifically noted, the percentage of enrichment refers to the percentage of deuterium present in the compound.

In other embodiments, a compound of this invention has an isotopic enrichment factor for each deuterium present at a site designated at a potential site of deuteration on the compound of at least 3500 (52.5.% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6466.7 (97% deuterium incorporation), at least 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 52.5% while the other could be deuterated at 75%. The resulting compound would be considered to be a compound wherein the isotopic enrichment factor is at least 3500 (52.5%).

Because the natural abundance of deuterium is about 0.015%, a small percentage of naturally occurring compounds of herein (e.g., a compound of Formula I, Ia, Ib, or Ic) would be expected to have one naturally occurring compound with one deuterium present.

In some embodiments, the compounds herein (e.g., a compound of Formula I, Ia, Ib, or Ic) comprise an amount of deuterium-enrichment that is more than the amount of deuterium-enrichment present in naturally occurring compounds herein (e.g., a compound of Formula I, Ia, Ib, or Ic)

All percentages given for the amount of deuterium present are mole percentages.

It can be difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen may still be present. Deuterium-enriched can be achieved by either exchanging protons with deuterium or by synthesizing the molecule with enriched starting materials.

### Methods of Treatment

Provided herein, in certain embodiments, is a method of modulating TRPML ion channels, the method comprising administering to a patient in need thereof a compound described herein (e.g., a compound of Formula I, Ia, Ib, or Ic) or pharmaceutically acceptable salts, solvates, hydrates, tautomers, stereoisomers, isotopically labeled derivatives thereof, or a composition described herein.

Provided herein, in certain embodiments, is a method of treating a disease or disorder that can be treated by modulation of TRPML ion channels, the method comprising administering to a patient in need thereof a compound described herein (e.g., a compound of Formula I, Ia, Ib, or Ic) or pharmaceutically acceptable salts, solvates, hydrates, tautomers, stereoisomers, isotopically labeled derivatives thereof, or a composition described herein.

Provided herein, in certain embodiments, is a method of treating a disease or disorder that can be treated by activation of TRPML ion channels, the method comprising administering to a patient in need thereof a compound described herein (e.g., a compound of Formula I, Ia, Ib, or Ic) or pharmaceutically acceptable salts, solvates, hydrates, tautomers, stereoisomers, isotopically labeled derivatives thereof, or a composition described herein.

Provided herein, in certain embodiments, is a method of treating a disease or disorder that can be treated by activation of TRPML1, the method comprising administering to a patient in need thereof a compound described herein (e.g., a compound of Formula I, Ia, Ib, or Ic) or pharmaceutically acceptable salts, solvates, hydrates, tautomers, stereoisomers, isotopically labeled derivatives thereof, or a composition described herein.

In addition to compounds of Formula I, Ia, Ib, or Ic, modulators of the TRPML channels have been reported in several publications, including WO2018005713 and WO2018208630, which are incorporated herein in their entirety.

In some embodiments, the TRPML ion channel is TRPML1. In some embodiments, the TRPML ion channel is TRPML2. In some embodiments, the TRPML ion channel is TRPML3.

In some embodiments, the compound is a modulator of TRPML1. In some embodiments, the compound is a modulator of TRPML2. In some embodiments, the compound is a modulator of TRPML3.

In some embodiments, modulation of the TRPML ion channel comprises activation of the ion channel.

In some embodiments, the disease or disorder is a ciliopathy (e.g., polycystic kidney disease). Exemplary ciliopathies include, but not limited to, polycystic kidney disease, pancreatic cysts in polycystic kidney disease, Bardet-Biedl syndrome, nephronophthisis, Joubert Syndrome, Mecke-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy, Ellis van Creveld syndrome, Sensenbrenner syndrome, and primary ciliary dyskinesia.

In an aspect, provided is a method of treating a disorder which can be treated by modulation of lysosomes, the method comprising administering to a patient in need thereof a therapeutically effective amount of a pharmaceutical composition of the disclosure or a compound of the disclosure.

In an aspect, provided is a method of treating a disorder selected from the group consisting of a ciliopathy, neurodegenerative disease, lysosomal storage disorder, lysosomal transport disorder, glycogen storage disorder, cholesteryl ester storage disease, a muscular disease (e.g., muscular dystrophy), a disease related to aging (e.g., photo aging of the skin), macular degeneration (e.g., Stargardt's or age-related), and cancer (e.g., cancers of the blood, brain, bone, lung, liver, kidney, bladder, stomach, breast, prostate, ovary, testes, colon, pancreas, or skin), the method comprising administering to a patient in need thereof a therapeutically effective amount of a pharmaceutical composition of the disclosure or a compound of the disclosure.

In some embodiments, the disorder is a ciliopathy.

In some embodiments, the ciliopathy is selected from the group consisting of polycystic kidney disease, pancreatic cysts in polycystic kidney disease, Bardet-Biedl syndrome, nephronophthisis, Joubert Syndrome, Mecke-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy, Ellis van Creveld syndrome, Sensenbrenner syndrome, and primary ciliary dyskinesia.

In some embodiments, the disorder is polycystic kidney disease. In some embodiments, the disorder is autosomal dominant polycystic kidney disease, autosomal recessive polycystic kidney disease, or pancreatic cysts associated with autosomal dominant polycystic kidney disease. In some embodiments, the disorder is autosomal dominant polycystic kidney disease. In some embodiments, the disorder is a neurodegenerative disorder.

In some embodiments, the neurodegenerative disorder is selected from the group consisting of Parkinson's disease, GBA-Parkinson's disease, LRRK2 Parkinson's disease, Huntington's disease, amyotrophic lateral sclerosis (ALS), Alzheimer's disease, progressive supranuclear palsy, frontotemporal dementia, FTDP-17, corticobasal degeneration, Lewy body dementia, Pick's disease, and multi system atrophy.

In some embodiments, the disorder is a lysosomal storage disorder.

In some embodiments, the lysosomal storage disorder is selected from the group consisting of Niemann-Pick disease, Gaucher's disease, neuronopathic Gaucher's disease, sphingolipidoses, Farber disease, Krabbe disease, Galactosialidosis, gangliosidoses, Gaucher Disease, Lysosomal acid lipase deficiency, sulfatidoses, mucopolysaccharidoses, mucolipidoses, lipidoses, and oligosaccharidoses.

In some embodiments, the lysosomal storage disorder is selected from the group consisting of sphingolipidoses, Farber disease, Krabbe disease, Galactosialidosis, Fabry disease, Schindler disease, beta-galactosidase disorder, GM1 gangliosidosis, GM2 gangliosidosis AB variant, GM2 gangliosidosis activator deficiency, Sandhoff disease, Tay-Sachs disease, Gaucher disease, lysosomal acid lipase deficiency, Niemann-Pick disease, metachromatic leukodystrophy, Saposin B deficiency, multiple sulfatase deficiency, Hurler syndrome, Scheie sundrome, Hurler-Scheie syndrome, Hunter syndrome, Sanfilippo syndrome, Morquio syndrome, Maroteaux-Lamy syndrome, Sly syndrome, hyaluronidase deficiency, sialidosis, I-cell disease, pseudo-Hurler polydystrophy, phosphotransferease deficiency, mucolipidin 1 deficiency, Santavuori-Haltia disease, Jansky-Bielchowsky disease, Batten-Spielmeyer-Vogt disease, Kufs disease, Finnish variant neuronal ceroid lipfuscinosis, late infantile variant neuronal ceroid lipfuscinosis, type 7 neuronal ceroid lipfuscinosis, northern epilepsy neuronal ceroid lipfuscinosis, Turkish late infantile neuronal ceroid lipfuscinosis, German/Serbian late infantile neuronal ceroid lipfuscinosis, congential cathepsin D deficiency, Wolman disease, alpha-mannosidosis, beta-mannosidosis, aspartylgluosaminuria, and fucosidosis.

In some embodiments, the lysosomal storage disorder is selected from the group consisting of Niemann-Pick disease, Gaucher's disease, and neuronopathic Gaucher's disease.

In some embodiments, the disorder is a lysosomal transport disease selected from the group consisting of cystinosis, pycnodysostosis, Salla disease, sialic acid storage disease, and infantile free sialic acid storage disease.

In some embodiments, the disorder is a glycogen storage disease selected from the group consisting of Pompe disease and Danon disease.

In an aspect, provided is a method of treating a ciliopathy disorder, the method comprising administering to a patient in need thereof a therapeutically effective amount of a compound capable of modulating TRPML, or a therapeutically effective amount of a pharmaceutical composition comprising the compound and a pharmaceutically acceptable excipient.

In some embodiments, the compound is selected from the compounds disclosed in the specification.

In some embodiments, the ciliopathy is selected from the group consisting of polycystic kidney disease, pancreatic cysts in polycystic kidney disease, Bardet-Biedl syndrome, nephronophthisis, Joubert Syndrome, Meckel-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy, Ellis van Creveld syndrome, Sensenbrenner syndrome, and primary ciliary dyskinesia.

In some embodiments, the disorder is polycystic kidney disease

In some embodiments, the disorder is autosomal dominant polycystic kidney disease, autosomal recessive polycystic kidney disease, or pancreatic cysts associated with autosomal dominant polycystic kidney disease.

In some embodiments, the disorder is autosomal dominant polycystic kidney disease.

In some embodiments, the method further comprises the use of a second therapeutic agent.

In some embodiments, the method is to treat a ciliopathy.

In some embodiments, the second therapeutic agent is selected from the group consisting of an mTOR inhibitor, V2 receptor antagonist, tyrosine kinase inhibitor, somatostatin analog, glucosylceramide synthase inhibitor, microRNA-17 inhibitor, siRNA against p53, KEAP1-Nrf2 activator, xanthine oxidase inhibitor, PPARγ agonist, metformin, and beta hydroxybutyrate.

In some embodiments, the second therapeutic agent is selected from the group consisting of tolvaptan, lixivaptan, mozavaptan, satavaptan, sirolimus, tacrolimus, everolimus, bosutinib, tesavatinib, imatinib, gefitinib, erlotinib, dasatinib, octreotide, pasireotide, venglustat, eliglustat, miglustat, microRNA-17 inhibitor, bardoxolone methyl, allopurinol, oxypurinol, pioglitazone, rosiglitazone, lobeglitazone, metformin, and beta hydroxybutyrate. In some embodiments, the second agent is tolvaptan.

In some embodiments, the second therapeutic agent is selected from the group consisting of an immunomodulator, a calcineurin inhibitor, a renin angiotensin aldosterone system inhibitor, an antiproliferative agent, an alkylating agent, a corticosteroid, an angiotensin converting enzyme inhibitor, an adrenocorticotropic hormone stimulant, an angiotensin receptor blocker, a sodium-glucose transport protein 2 inhibitor, a dual sodium-glucose transport protein 1/2 inhibitor, a nuclear Factor-1 (erythroid-derived 2)-like 2 agonist, a chemokine receptor 2 inhibitor, a chemokine receptor 5 inhibitor, an endothelin 1 receptor antagonist, a beta blocker, a mineralocorticoid receptor antagonist, a loop or thiazide diuretic, a calcium channel blocker, a statin, a short-intermediate or long-acting insulin, a dipeptidyl peptidase 4 inhibitor, a glucagon-like peptide 1 receptor agonist, a sulfonylurea, an apoptosis signal-regulating kinase-1, a chymase inhibitor, a selective gly cation inhibitor, a renin inhibitor, an interleukin-33 inhibitor, a farnesoid X receptor agonist, a soluble guanylate cyclase stimulator, a thromboxane receptor antagonist, a xanthine oxidase inhibitor, an erythropoietin receptor agonist, a cannabinoid receptor type 1 inverse agonist, a NADPH oxidase inhibitor, an anti-vascular endothelial growth factor B, an anti-fibrotic agent, a neprilysin inhibitor, a dual CD80/CD86 inhibitor, a CD40 antagonist, a cellular cholesterol and lipid blocker, a PDGFR antagonist, a Slit guidance ligand 2, an APOL1 inhibitor, an Nrl2 activator/NF-kB inhibitor, a somatostatin receptor agonist, a PPAR gamma agonist, a AMP activated protein kinase stimulator a tyrosine kinase inhibitor, a glucosylceramide synthase inhibitor, an arginine vasopressin receptor 2 antagonist, a xanthine oxidase inhibitor, a vasopressin receptor 2 antagonist, anti-amyloid beta antibodies, anti-Tau antibodies, anti-synuclein antibodies, dopamine precursors (e.g. L-DOPA), dopamine agonists (e. g. bromocriptine, cabergoline, pergolide, pramipexole and apomorphine), MAO-B inhibitors (e.g rasagiline and selegiline), anticholinergics (e g. orphenadrine, procyclidine and trihexyphenidyl), enhancers of b-glucocerebrosidase activity (e.g. ambroxol and afegostat), amantadine, and agents capable of treating Alzheimer's (e.g., acetylcholinesterase inhibitors such as tacrine, rivastigmine, galantamine, donepezil, and NMDA receptor antagonists such as memantine).

In some embodiments, the second therapeutic agent is selected from the group consisting of COX inhibitors including arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolimetin, zomepirac, clopinac, indomethacin and sulindac) and enolic acids (phenylbutazone, oxyphenbutazone, azapropazone, feprazone, piroxicam, and isoxicam; treatments for pulmonary hypertension including prostanoids (epoprostenol, iloprost, and treprostinil), endothelin receptor antagonists (bosentan, ambrisentan, and macitentan), phosphodiesterase-5 inhibitors (sildenafil and tadalafil), and sGC stimulators (riociguat); rho-kinase inhibitors, such as Y-27632, fasudil, and H-1152P; epoprostenol derivatives, such as prostacyclin, treprostinil, beraprost, and iloprost; serotonin blockers, such as sarpogrelate; endothelin receptor antagonists, such as besentan, sitaxsentan, ambrisentan, and TBC3711; PDE inhibitors, such as sildenafil, tadalafil, udenafil, and vardenafil; soluble gunaylate cyclase inhibitors such as riociguat and vericiguat; calcium channel blockers, such as amlodipine, bepridil, clentiazem, diltiazem, fendiline, gallopamil, mibefradil, prenylamine, semotiadil, terodiline, verapamil, aranidipine, bamidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, and perhexiline, tyrosine kinase inhibitors, such as imatinib; inhaled nitric oxide and nitric oxide-donating agents, such as inhaled nitrite: IκB inhibitors, such as IMD 1041; prostacyclin receptor agonists, such as selexipag; stimulators of hematopoiesis, such as TXA 127 (angiotensin (1-7)), darbepoetin alfa, erythropoetin, and epoetin alfa; anticoagulants and platelet-inhibiting agents; and diuretics, dietary and nutritional supplements such as acetyl-L-carnitine, octacosanol, evening primrose oil, vitamin B6, tyrosine, phenylalanine, vitamin C, L-dopa; immunosuppressants (for transplants and autoimmune-related RKD); anti-hypertensive drugs (for high blood pressure-related RKD, e.g., angiotensin-converting enzyme inhibitors and angiotensin receptor blockers); insulin (for diabetic RKD); lipid/cholesterol-lowering agents (e.g, HMG-CoA reductase inhibitors such as atorvastatin or simvastatin); and treatments for hyperphosphatemia or hyperparathyroidism associated with CKD (e.g, sevelamer acetate, cinacalcet).

The present disclosure further provides pharmaceutical compositions comprising a compound provided herein, or a pharmaceutically acceptable salt thereof, and at least one pharmaceutically acceptable carrier. The present disclosure further provides methods of modulating TRPML in a subject, the method comprising administering to the subject a therapeutically effective amount of a compound provided herein, or a pharmaceutically acceptable salt thereof.

The present disclosure further provides a method of treating a disease or disorder in a subject, the method comprising:
(a) detecting a disease or disorder associated with TRPML; and
(b) administering to the subject a therapeutically effective amount of a compound provided herein, or a pharmaceutically acceptable salt thereof.

In certain embodiments, exemplary compounds of Formula (I) or (II) include the compounds described in Table 1 and in the Examples, as well as pharmaceutically acceptable salts, solvates, hydrates, tautomers, and stereoisomers thereof.

Accordingly, the present disclosure provides compounds useful for treating ciliopathies and related diseases.

Compounds that modulate TRPML channels may be useful in the prophylaxis and treatment of any of the foregoing injuries, diseases, disorders, or conditions. In addition to in vitro assays of the activity of these compounds, their efficacy can be readily tested in one or more animal models.

This disclosure is not limited in its application to the details of the methods and compositions described herein. Also, the phraseology and terminology used herein is for the purpose of description and should not be regarded as limiting.

### Pharmaceutical Compositions and Routes of Administration

The present disclosure provides pharmaceutical compositions comprising a compound provided herein, or a pharmaceutically acceptable salt thereof, and at least one pharmaceutically acceptable carrier. Also provided herein are methods of modulating TRPML channels in a subject, the method comprising administering to the subject a therapeutically effective amount of a compound provided herein, or a pharmaceutically acceptable salt thereof.

In certain embodiments, pharmaceutical compositions containing compounds described herein such as a compound of Formula I, Ia, Ib, or Ic, or pharmaceutically acceptable salt thereof can be used to treat or ameliorate a disorder described herein, for example, a ciliopathy.

The amount and concentration of compounds of Formula I, Ia, Ib, or Ic in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

While it is possible for a compound disclosed herein to be administered alone, it is preferable to administer the compound as a pharmaceutical formulation, where the compound is combined with one or more pharmaceutically acceptable diluents, excipients or carriers. The compounds according to the disclosure may be formulated for administration in any convenient way for use in human or veterinary medicine. In certain embodiments, the compound included in the pharmaceutical preparation may be active itself, or may be a prodrug, e.g., capable of being converted to an active compound in a physiological setting. Regardless of the route of administration selected, the compounds of the present disclosure, which may be used in a suitable hydrated form, and/or the pharmaceutical compositions of the present disclosure, are formulated into pharmaceutically acceptable dosage forms such as described below or by other conventional methods known to those of skill in the art.

Thus, another aspect of the present disclosure provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present disclosure may be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; (9) nasally; or (10) intrathecally. Additionally, compounds can be implanted into a patient or injected using a drug delivery system. See, for example, Urquhart, et al., (1994) Ann Rev Pharmacol Toxicol 24:199-236; Lewis, ed. “Controlled Release of Pesticides and Pharmaceuticals” (Plenum Press, New York, 1981); U.S. Pat. No. 3,773,919; and U.S. Pat. No. 353,270,960.

The phrase “therapeutically effective amount” as used herein means that amount of a compound, material, or composition comprising a compound of the present disclosure, which is effective for producing some desired therapeutic effect, e.g., by modulating EHMT1 or EHMT2, in at least a sub-population of cells in an animal and thereby blocking the biological consequences of that function in the treated cells, at a reasonable benefit/risk ratio applicable to any medical treatment.

The phrases “systemic administration,” “administered systemically,” “peripheral administration” and “administered peripherally” as used herein mean the administration of a compound, drug, or other material other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) cyclodextrins such as Captisol®; and (22) other non-toxic compatible substances employed in pharmaceutical formulations.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for the present disclosure.

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal, vaginal, or urethral administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively, or additionally, compositions can be formulated for delivery via a catheter, stent, wire, or other intraluminal device. Delivery via such devices may be especially useful for delivery to the heart, lung, bladder, urethra, ureter, rectum, or intestine. Furthermore, compositions can be formulated for delivery via a dialysis port.

Ophthalmic formulations, eye ointments, powders, solutions and the like, are also contemplated as being within the scope of this disclosure.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the compositions are administered by intravenous infusion or injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions of this disclosure suitable for parenteral administration comprise one or more compounds of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present disclosure are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the disclosure to animal feed is preferably accomplished by preparing an appropriate feed premix containing the active compound in an effective amount and incorporating the premix into the complete ration. Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Ore., U.S.A., 1977).

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with neurodegenerative disease or disorder, cancer, or viral infections.

In addition, the methods described herein can be used to treat domesticated animals and/or pets. A subject can be male or female. A subject can be one who has been previously diagnosed with or identified as suffering from or having a neurodegenerative disease or disorder, a disease or disorder associated with cancer, a disease or disorder associated with viral infection, or one or more complications related to such diseases or disorders but need not have already undergone treatment.

### Dosages

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present disclosure employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

The compound and the pharmaceutically active agent can be administrated to the subject in the same pharmaceutical composition or in different pharmaceutical compositions (at the same time or at different times). When administrated at different times, the compound and the pharmaceutically active agent can be administered within 5 minutes, 10 minutes, 20 minutes, 60 minutes, 2 hours, 3 hours, 4, hours, 8 hours, 12 hours, 24 hours of administration of the other agent. When the compound and the pharmaceutically active agent are administered in different pharmaceutical compositions, routes of administration can be different.

The amount of compound that can be combined with a carrier material to produce a single dosage form will generally be that amount of the compound that produces a therapeutic effect. Generally, out of one hundred percent, this amount will range from about 0.1% to 99% of compound, preferably from about 5% to about 70%, most preferably from 10% to about 30%.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the EC₅₀ (i.e., the concentration of the therapeutic which achieves a half-maximal effect) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the drugs. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

The present disclosure contemplates formulation of the subject compounds in any of the aforementioned pharmaceutical compositions and preparations. Furthermore, the present disclosure contemplates administration via any of the foregoing routes of administration. One of skill in the art can select the appropriate formulation and route of administration based on the condition being treated and the overall health, age, and size of the patient being treated.

### Selected Chemical Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, propyl, butyl, pentyl, and hexyl

For compounds of the disclosure in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

In case a compound of the present disclosure is depicted in form of a chemical name and as a formula in case of any discrepancy the formula shall prevail.

An asterisk or wavy line may be used in sub-formulas to indicate the bond which is connected to the core molecule as defined.

The term “substituted,” as used herein, means that any one or more hydrogens on the designated atom, usually a carbon, oxygen, or nitrogen atom, is replaced with a selection from the indicated group, provided that the designated atom's normal valency is not exceeded, and that the substitution results in a stable compound. When a substituent is keto or oxo (i.e., ═O), then 2 hydrogens on the atom are replaced Ring double bonds, as used herein, are double bonds that are formed between two adjacent ring atoms (e.g., C═C, C═N, N═N, etc.).

As used herein, “alkyl” is intended to include both branched and straight-chain saturated aliphatic hydrocarbon groups having the specified number of carbon atoms. For example, C₁₋₄ alkyl is intended to include C₁, C₂, C₃, and C₄. C₁₋₆ alkyl is intended to include C₁ C₂, C₃, C₄, C₅, and C₆ alkyl groups and C₁₋₈ alkyl is intended to include C₁, C₂, C₃, C₄, C₅, C₆, C₇, and C₈. Some examples of alkyl include, but are not limited to, methyl, ethyl, n-propyl, iso-propyl, n-butyl, s-butyl, t-butyl, n-pentyl, s-pentyl, n-hexyl, n-heptyl, and n-octyl.

As used herein, “alkenyl” is intended to include hydrocarbon chains of either straight or branched configuration and one or more unsaturated carbon-carbon bond that can occur in any stable point along the chain, such as ethenyl and propenyl. For example, C₂₋₆ alkenyl is intended to include C₂, C₃, C₄, C₅, and C₆ alkenyl groups and C₂₋₈ alkenyl is intended to include C₂, C₃, C₄, C₅, C₆, C₇, and C₈ alkenyl groups.

As used herein, “alkylene” is intended to include moieties which are diradicals, i.e., having two points of attachment. A non-limiting example of such an alkylene moiety that is a diradical is —CH₂CH₂—, i.e., a C₂ alkyl group that is covalently bonded via each terminal carbon atom to the remainder of the molecule. The alkylene diradicals are also known as “alkylenyl” radicals. Alkylene groups can be saturated or unsaturated (e.g., containing —CH═CH— or —C≡C— subunits), at one or several positions. In some embodiments, alkylene groups include 1 to 9 carbon atoms (for example, 1 to 6 carbon atoms, 1 to 4 carbon atoms, or 1 to 2 carbon atoms). Some examples of alkylene groups include, but are not limited to, methylene, ethylene, n-propylene, iso-propylene, n-butylene, iso-butylene, sec-butylene, tert-butylene, n-pentylene, iso-pentylene, sec-pentylene and neo-pentylene.

As used herein, “cycloalkyl” is intended to include saturated or unsaturated nonaromatic ring groups, such as cyclopropyl, cyclobutyl, cyclopentyl, or cyclohexyl. For example, the term “C₃₋₈ cycloalkyl” is intended to include C₃, C₄, C₅, C₆, C₇, and C₈ cycloalkyl groups. Cycloalkyls may include multiple spiro- or fused or bridged rings. For example, cycloalkyl can include, but is not limited to, spiro butyl, pentyl, hexyl, heptyl, octyl, nonyl, or decyl groups, bicyclo butyl, pentyl, hexyl, heptyl, octyl, nonyl, or decyl groups, adamantyl groups, and norbornyl groups.

As used herein, the term “heterocycloalkyl” refers to a saturated or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, or Se), unless specified otherwise. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, tetrahyrofuranyl, oxiranyl, azetidinyl, oxetanyl, thietanyl, 1,2,3,6-tetrahydropyridinyl, tetrahydropyranyl, dihydropyranyl, pyranyl, morpholinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl and the like.

As used herein, “amine” or “amino” refers to unsubstituted —H₂ unless otherwise specified. As used herein, “halo” or “halogen” refers to fluoro, chloro, bromo, and iodo substituents.

As used herein, “haloalkyl” is intended to include both branched and straight-chain saturated aliphatic hydrocarbon groups having the specified number of carbon atoms, substituted with one or more halogen (for example —CᵥF_{w}H_{2v·w+1} wherein v=1 to 3 and w=1 to (2v+1)). Examples of haloalkyl include, but are not limited to, trifluoromethyl, trichloromethyl, pentafluoroethyl, and pentachloroethyl.

The term “haloalkoxy” as used herein refers to an alkoxy group, as defined herein, which is substituted one or more halogen Examples of haloalkoxy groups include, but are not limited to, trifluoromethoxy, difluoromethoxy, pentafluoroethoxy, trichloromethoxy, etc.

As used herein, “alkoxyl” or “alkoxy” refers to an alkyl group as defined above with the indicated number of carbon atoms attached through an oxygen bridge. C₁₋₆ alkoxy, is intended to include C₁, C₂, C₃, C₄, C₅, and C₆ alkoxy groups. C₁₋₈ alkoxy, is intended to include C₁, C₂, C₃, C₄, C₅, C₆, C₇, and C₈ alkoxy groups Examples of alkoxy include, but are not limited to, methoxy, ethoxy, n-propoxy, i-propoxy, n-butoxy, s-butoxy, t-butoxy, n-pentoxy, s-pentoxy, n-heptoxy, and n-octoxy.

As used herein, “aryl” includes groups with aromaticity, including “conjugated,” or multicyclic systems with at least one aromatic ring and do not contain any heteroatom in the ring structure. Aryl may be monocyclic or polycyclic (e.g., having 2, 3 or 4 fused rings). The term “Cₙ₋ₘ aryl” refers to an aryl group having from n to m ring carbon atoms. In some embodiments, aryl groups have from 6 to 10 carbon atoms. In some embodiments, the aryl group is phenyl or naphthyl.

As used herein, the terms “aromatic heterocycle,” “aromatic heterocyclic” or “heteroaryl” ring are intended to mean a stable 5, 6, 7, 8, 9, 10, 11, or 12-membered monocyclic or bicyclic aromatic ring which consists of carbon atoms and one or more heteroatoms, e.g., 1 or 1-2 or 1-3 or 1-4 or 1-5 or 1-6 heteroatoms, independently selected from nitrogen, oxygen, and sulfur. In the case of bicyclic aromatic heterocyclic or heterocycle or heteroaryl rings, only one of the two rings needs to be aromatic (e.g., 2,3-dihydroindole), though both can be (e.g., quinoline). The second ring can also be fused or bridged as defined above for heterocycles. The nitrogen atom can be substituted or unsubstituted (i.e., N or R wherein R is H or another substituent, as defined). The nitrogen and sulfur heteroatoms can optionally be oxidized (i.e., N→O and S(O)_{P}, wherein p=1 or 2). In certain compounds, the total number of S and O atoms in the aromatic heterocycle is not more than 1. Examples of aromatic heterocycles, aromatic heterocyclics or heteroaryls include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, benzooxadiazoly, carbazolyl, 4aH-carbazolyl, carbolinyl, cinnolinyl, furazanyl, imidazolyl, imidazolonyl, 1H-indazolyl, indolizinyl, indolyl, 3H-indolyl, isobenzofuranyl, isochromanyl, isoindazolyl, isoindolyl, isoquinolinyl, isothiazolyl, isoxazolyl, methylbenztriazolyl, methylfuranyl, methylimidazolyl, methylthiazolyl, naphthyridinyl, oxadiazolyl, 1,2,3-oxadiazolyl, 1,2,4-oxadiazolyl, 1,2,5-oxadiazolyl, 1,3,4-oxadiazolyl, oxazolyl, phenanthridinyl, phenanthrolinyl, phenazinyl, phenothiazinyl, phenoxathinyl, phenoxazinyl, phthalazinyl, pteridinyl, purinyl, pyrazinyl, pyrazolyl, pyridazinyl, pyridooxazolyl, pyridoimidazolyl, pyridothiazolyl, pyridinyl, pyridinonyl, pyridyl, pyrimidinyl, 2H-pyrrolyl, pyrrolyl, quinazolinyl, quinolinyl, 4H-quinolizinyl, quinoxalinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, triazolopyrimidinyl, 1,2,3-triazolyl, 1,2,4-triazolyl, 1,2,5-triazolyl, and 1,3,4-triazolyl.

The term “hydroxyalkyl” means an alkyl group as defined above, where the alkyl group is substituted with one or more OH groups. Examples of hydroxyalkyl groups include HO—CH₂—, HO—CH₂—CH₂— and CH₃—CH(OH)—.

The term “cyano” as used herein means a substituent having a carbon atom joined to a nitrogen atom by a triple bond, i.e., C≡N.

As used herein, “oxo” is means a “C═O” group.

As used herein, the phrase “pharmaceutically acceptable” refers to those compounds or tautomers thereof, or salts thereof, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein, “pharmaceutically acceptable salts” refer to derivatives of the disclosed compounds or tautomers thereof, wherein the parent compound or a tautomer thereof, is modified by making of the acid or base salts thereof of the parent compound or a tautomer thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound, or a tautomer thereof, formed, for example, from non-toxic inorganic or organic acids. For example, such conventional non-toxic salts include, but are not limited to, those derived from inorganic and organic acids selected from 2-acetoxybenzoic, 2-hydroxy ethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, and toluene sulfonic.

The pharmaceutically acceptable salts of the present disclosure can be synthesized from the parent compound or a tautomer thereof that contains a basic or acidic moiety by conventional chemical methods Generally, such pharmaceutically acceptable salts can be prepared by reacting the free acid or base forms of these compounds or tautomers thereof with a stoichiometric amount of the appropriate base or acid in water or in an organic solvent, or in a mixture of the two: generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, PA, USA, p. 1445 (1990).

As used herein, “stable compound” and “stable structure” are meant to indicate a compound that is sufficiently robust to survive isolation to a useful degree of purity from a reaction mixture, and formulation into an efficacious therapeutic agent.

As used herein, the term “treating” refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing, or preventing the further development of a disorder, and/or reducing the severity of symptoms that will or are expected to develop.

As used herein, “unsaturated” refers to compounds having at least one degree of unsaturation (e.g., at least one multiple bond) and includes partially and fully unsaturated compounds.

As used herein, the term “effective amount” refers to an amount of a compound or a pharmaceutically acceptable salt of the compound or tautomer (including combinations of compounds and/or tautomers thereof, and/or pharmaceutically acceptable salts of said compound or tautomer) of the present disclosure that is effective when administered alone or in combination as an antimicrobial agent. For example, an effective amount refers to an amount of the compound or tautomer thereof, or a pharmaceutically acceptable salt said compound or tautomer that is present in a composition, a formulation given to a recipient patient or subject sufficient to elicit biological activity.

In the specification, the singular forms also include the plural, unless the context clearly dictates otherwise. Unless defined otherwise, all technical and scientific terms used herein have the same meaning as commonly understood by one of ordinary skill in the art to which this disclosure belongs. In the case of conflict, the present specification will control. As used herein, “mammal” refers to human and non-human patients.

As used herein, the term “formulae of the disclosure” or “formulae disclosed herein” includes one or more of the Formulas I, its subformulas Ia, Ib, or Ic, and further subformulas thereof.

As used herein, the term “compound of the disclosure” or “compound disclosed herein” includes one or more compounds of the formulae of the disclosure or a compound explicitly disclosed herein.

All percentages and ratios used herein, unless otherwise indicated, are by weight.

Throughout the description, where compositions are described as having, including, or comprising specific components, or where processes are described as having, including, or comprising specific process steps, it is contemplated that compositions of the present disclosure also consist essentially of or consist of the recited components, and that the processes of the present disclosure also consist essentially of, or consist of, the recited processing steps. Further, it should be understood that the order of steps or order for performing certain actions are immaterial so long as the disclosure remains operable. Moreover, two or more steps or actions can be conducted simultaneously.

Contemplated equivalents of the compounds described above include compounds which otherwise correspond thereto, and which have the same general properties thereof (e.g., the ability to modulate TRPML), wherein one or more simple variations of substituents are made which do not adversely affect the efficacy of the compound. In general, the compounds of the present disclosure may be prepared by the methods illustrated in the general reaction schemes as, for example, described below, or by modifications thereof, using readily available starting materials, reagents and conventional synthesis procedures. In these reactions, it is also possible to make use of variants which are in themselves known but are not mentioned here.

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The term, “treat” or “treatment,” as used herein, refers to the application or administration of a compound, alone or in combination with, an additional agent to a subject, e.g., a subject who has a disorder (e.g., a disorder as described herein), a symptom of a disorder, or a predisposition toward a disorder, with the purpose to cure, heal, alleviate, relieve, alter, remedy, ameliorate, improve or affect the disorder.

As used herein, the term “subject” is intended to include human and non-human animals. Exemplary human subjects include a human subject having a disorder, e.g., a disorder described herein. The term “non-human animals” of the disclosure includes all vertebrates, e.g., non-mammals (such as chickens, amphibians, reptiles) and mammals, such as non-human primates, domesticated and/or agriculturally useful animals, e.g., sheep, dog, cat, cow, pig, etc.

The terms “antagonist” and “inhibitor” are used interchangeably to refer to an agent that decreases or suppresses a biological activity.

The terms “activator” and “agonist” are used interchangeably to refer to an agent that increases or initiates a biological activity.

The term “hydrate” as used herein, refers to a compound formed by the union of water with the parent compound.

The term “preventing,” when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “solvate” as used herein, refers to a compound formed by solvation (e.g., a compound formed by the combination of solvent molecules with molecules or ions of the solute). Another aspect of the disclosure features a pharmaceutical preparation suitable for use in a human patient, or for veterinary use, comprising an effective amount of a compound of the formulae of the disclosure (or a salt thereof, or a solvate, hydrate, oxidative metabolite or prodrug of the compound or its salt), and one or more pharmaceutically acceptable excipients. The disclosure further contemplates the use of compounds of the formulae of the disclosure in the manufacture of a medicament or pharmaceutical preparation to treat or reduce the symptoms of any of the diseases or conditions provided in the specification. The compounds of the formulae of the disclosure for use in treating a particular disease or condition can be formulated for administration via a route appropriate for the particular disease or condition.

Compounds of the formulae of the disclosure can be administered alone or in combination with another therapeutic agent. For instance, the compounds of the formulae of the disclosure can be administered conjointly with one or more of an agent for treating polycystic kidney disease, etc.

Compounds of the formulae of the disclosure can be administered topically, orally, transdermally, rectally, vaginally, parentally, intranasally, intrapulmonary, intraocularly, intravenously, intramuscularly, intraarterially, intrathecally, intracapsularly, intraorbitally, intracardiacly, intradermally, intraperitoneally, transtracheally, subcutaneously, subcuticularly, intraarticularly, subcapsularly, subarachnoidly, intraspinally, intrasternally, sublingually, or by inhalation.

In some embodiments, compounds of Formula I, Ia, Ib, or Ic can be administered topically.

In some embodiments, compounds of Formula I, Ia, Ib, or Ic can be administered orally.

In some embodiments, compounds of Formula I, Ia, Ib, or Ic can be administered parentally.

Compounds of Formula I, Ia, Ib, or Ic include molecules having an aqueous solubility suitable for oral or parenteral (e.g., intravenous) administration leading to or resulting in the treatment of a disorder described herein, for example the treatment of pain. In some embodiments, the compound is formulated into a composition suitable for oral administration.

In some embodiments, a compound of Formula I, Ia, Ib, or Ic can be administered as part of an oral or parenteral (e.g., intravenous) pharmaceutical composition to treat a disorder described herein in a therapeutically effective manner.

Certain compounds disclosed herein may exist in particular geometric or stereoisomeric forms. The present disclosure contemplates all such compounds, including cis- and trans-isomers, R- and S-enantiomers, diastereomers, (d)-isomers, (1)-isomers, the racemic mixtures thereof, and other mixtures thereof, as falling within the scope of the disclosure. For example, if one chiral center is present in a molecule, the disclosure includes racemic mixtures, enantiomerically enriched mixtures, and substantially enantiomerically or diastereomerically pure compounds. The composition can contain, e.g., more than 50%, more than 60%, more than 70%, more than 80%, more than 90%, more than 95%, or more than 99% of a single enantiomer or diastereomer. Additional asymmetric carbon atoms may be present in a substituent such as an alkyl group. All such isomers, as well as mixtures thereof, are intended to be included in this disclosure.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer.

ee=(90−10)/100=80%.

Thus, a composition containing 90% of one enantiomer and 10% of the other enantiomer is said to have an enantiomeric excess of 80%.

The “diastereomeric excess” or “% diastereomeric excess” of a composition can be calculated using the equation shown below. In the example shown below a composition contains 90% of one diastereomer, and 10% of another enantiomer.

de=(90−10)/100=80%.

Thus, a composition containing 90% of one diastereomer and 10% of the other diastereomer is said to have a diastereomeric excess of 80%.

Certain compounds disclosed herein can exist in unsolvated forms as well as solvated forms, including hydrated forms. In general, the solvated forms are equivalent to unsolvated forms and are encompassed within the scope of the present disclosure. Certain compounds disclosed herein may exist in multiple crystalline or amorphous forms. In general, all physical forms are equivalent for the uses contemplated by the present disclosure and are intended to be within the scope of the present disclosure.

### EXAMPLES

Examples are provided below to facilitate a more complete understanding of the invention. The following examples illustrate exemplary modes of making and practicing the invention. However, the scope of the invention is not limited to specific embodiments disclosed in these Examples, which are for purposes of illustration only, since alternative methods can be utilized to obtain similar results.

General. All oxygen and/or moisture sensitive reactions were carried out under N₂ atmosphere in glassware that was flame-dried under vacuum (0.5 mmHg) and purged with N₂ prior to use. All reagents and solvents were purchased from commercial vendors and used as received or synthesized according to the footnoted references. NMR spectra were recorded on a Bruker 400 (400 MHz ¹H, 75 MHz ¹³C) or Varian (400 MHz ¹H, 75 MHz ¹³C) spectrometer. Proton and carbon chemical shifts are reported in ppm (δ) referenced to the NMR solvent. Data are reported as follows: chemical shifts, multiplicity (br=broad, s=singlet, t=triplet, q=quartet, m=multiplet; coupling constant(s) in Hz). Unless otherwise indicated NMR data were collected at 25° C. Flash chromatography was performed using 100-200 mesh Silica Gel. Liquid Chromatography/Mass Spectrometry (LCMS) was performed on Agilent 1200HPLC and 6110MS. Analytical thin layer chromatography (TLC) was performed on 0.2 mm silica gel plates. Visualization was accomplished with UV light and aqueous potassium permanganate (KMnO₄) stain followed by heating.

### LCMS Typical Conditions:

LC-MS conditions Instrument: LCMS2020 (E-LCMS 008) Column: Shim-pack GIST C18, 50*4.6 mm 5 um Mobile Phase: A: H2O (0.1% FA) B: CH₃CN Temperature: 35 ¿æ Flow rate: 2.5 mL/min Run time: 0.1 min@20% B, 1.7 min gradient (20-95% B), then 0.7 min@95% B, then 0.4 min@20% B Injection volume: 5 uL Detector: UV 220/254 nm Mass range: 100-1000 Scan: Positive/Negative.

### HPLC Typical Conditions:

Instrument: LC-20AD(E-LC 006) Column: YMC Triart C18, 50×4.6 mm, 5 um Mobile phase: Solvent A: H₂O/CH₃CN/TFA=90/10/0.1 Solvent B: H₂O/CH₃CN/TFA=10/90/0.1 Flow rate: 2.5 mL/min Run time 0.4 min@60 B, 3.4 m gradient (60-100% B), then 0.8 min@100% B. Temperature: 35° C. Detector: UV.

### Or

Instrument: LC-2010AT(E-LC 001) Column: Gemini, C18, 50×4.6 mm, 5 um Mobile phase: Solvent A: H₂O/CH₃CN/TFA=90/10/0.1 Solvent B: H₂O/CH₃CN/TFA=10/90/0.1 Flow rate: 2.5 mL/min Run time: 0.4 min@20% B, 3.4 min gradient (20-95% B), then 0.8 min@95% B. Temperature: 40° C. Detector: UV.

TABLE 2
Abbreviations

| | | | |
|---|---|---|---|
| AIBN | azobisisobutyronitrile | dtbbpy | 4,4′-di-tert-butyl-2,2′- |
| | | | dipyridyl |
| BAST | bis(2- | EA or | ethyl acetate |
| | methoxyethyl)aminosulfur | EtOAc | |
| | trifluoride | | |
| Bn | benzyl | EDCI | 1-ethyl-3-(3- |
| | | | dimethylaminopropyl)carbodiimide |
| BcOH | benzyl alcohol | ESI | electrospray ionization |
| Boc | t-butoxycarbonyl | EGTA | ethylene glycol-bis(β- |
| | | | aminoethyl ether)- |
| | | | N,N,N′,N′-tetraacetic acid |
| (Boc)₂O | di-tert-butyl dicarbonate | EtI | ethyl iodide |
| t-BuXphos | 2-di-t-butylphosphino- | EtOAc | ethyl acetate |
| | 2′,4′,6′-triisopropylbiphenyl | | |
| t-BuOK | potassium tert-butoxide | EtOH | ethanol |
| CDI | carbonyldiimidazole | Et₂Zn | diethylzinc |
| CMBP | (cyanomethylene)tributylphosphorane | Fe(acac)₃ | tris(acetylacetonato)iron(III) |
| COSY | correlation spectroscopy | GTP | guanosine-5′-triphospphate |
| Cu(OAc)₂ | copper(II) acetate | HATU | O-(7-azabenzotriazol-1-yl)- |
| | | | N,N,N′,N′- |
| | | | tetramethyluronium |
| | | | hexafluorophosphate |
| DAST | diethylaminosulfur trifluoride | HEPES | 4-(2-hydroxyethyl)-1- |
| | | | piperazineethanesulfonic acid |
| DCM | dichloromethane | ¹H NMR | proton nuclear magnetic |
| | | | resonance |
| DIAD | diisopropyl azodicarboxylate | HOAc | acetic acid |
| DIBALH | diisobutylaluminum hydride | HOBT | hydroxybenzotriazole |
| DIC | N,N′- | HPLC | high performance liquid |
| | methanediylidenedipropan-2- | | chromatography |
| | amine | | |
| DIEA | N,N-diisopropylethylamine | LCMS | liquid chromatography mass |
| | | | spectrometry |
| DIPEA | N,N-diisopropylethylamine | mCPBA | meta-chloroperbenzoic acid |
| DMA | N,N-dimethylacetamide | Me | methyl |
| DMAP | N,N-4- | MeOH | methanol |
| | dimethylaminopyridine | | |
| DME | 1,2-dimethoxyethane | MeI | methyl iodide |
| DMF | N,N-dimethylformamide | MsCl | methanesulfonyl chloride |
| DMSO | dimethylsulfoxide | Ms | methanesulfonyl |
| DPPA | diphenylphosphoryl azide | NBS | N-bromosuccinimide |
| dppf | 1,1′- | | |
| | bis(diphenylphosphino)ferrocene | | |
| dtbpf | 1,1′-bis(di-t- | | |
| | butylphosphino)ferrocene | | |
| NOE | nuclear Overhauser effect | SEM | 2- |
| | | | (trimethylsilyl)ethoxymethyl |
| PCC | Pyridinium chlorochromate | SFC | supercritical fluid |
| | | | chromatography |
| Pd₂(dba)₃ | tris(dibenzylideneacetone)dipalladium | Py—SO₃ | sulfur trioxide-pyridine |
| Pd(dppf)Cl₂ | 1,1- | TBAF | tetrabutylammonium fluoride |
| | bis(diphenylphosphino)ferrocene-palladium(II)dichloride | | |
| Pd(PPh₃)₄ | tetrakis(triphenylphosphine)palladium(0) | TBAI | tetrabutylammonium iodide |
| Pd-118 or | 1,1-bis(di-t- | TBDPS | tert-butyl-diphenylsilyl |
| Pd(dtbpf)Cl₂ | butylphosphino)ferrocene- | | |
| | palladium(II)dichloride | | |
| PE | petroleum ether | TBS | tert-butyl-dimethylsilyl |
| PhSiH₃ | phenylsilane | TEA | triethylamine |
| Py or pyr | pyridine | TFA | trifluoroacetic acid |
| POCl₃ | phosphoryl chloride | TFAA | trifluoroacetic anhydride |
| PPh₃ | triphenylphosphine | THF | tetrahydrofuran |
| RhH(CO)(PPh₃)₃ | tris(triphenylphosphine)rhodium | TLC | thin layer chromatography |
| | carbonyl hydride | | |
| rt | room temperature | Tol | toluene |
| | | Ts | p-tolueneslfonyl |
| | | TsCl | 4-toluenesulfonyl chloride |
| | | TsOH | tosylic acid |
| | | x-Phos | dicyclohexyl[2′,4′,6′- |
| | | | tris(propan-2-yl)[1,1′- |
| | | | biphenyl]-2-yl]phosphane |

### Example 1. Synthesis of tert-butyl 4-(5-cyclopropyl-7-(3,5-difluorophenyl)-7H-pyrrolo[2,3-d]pyrimidin-4-yl)piperazine-1-carboxylate (Compound 146)

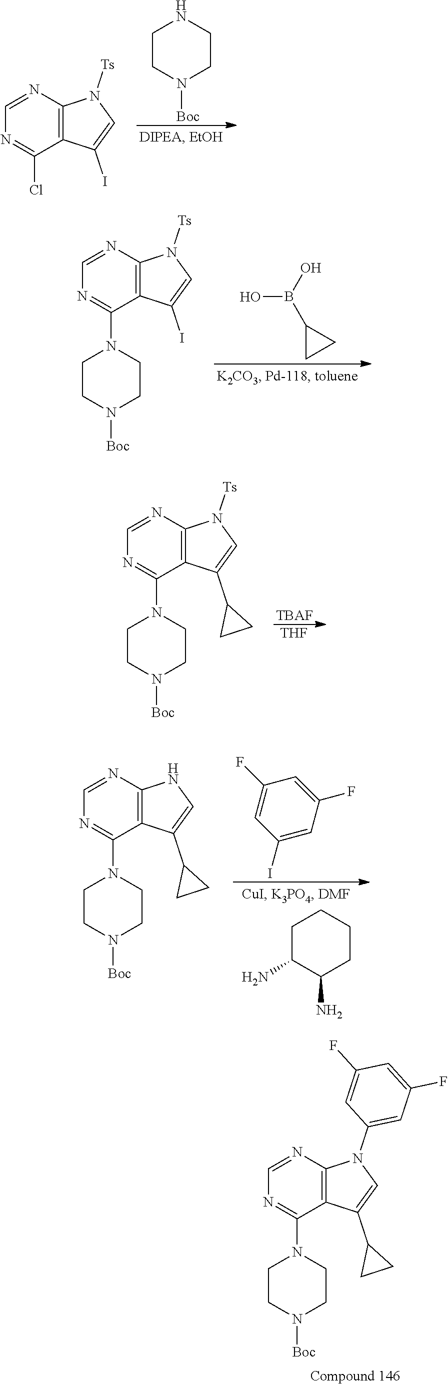

### Step 1. tert-Butyl 4-(5-iodo-7-tosyl-7H-pyrrolo[2,3-d]pyrimidin-4-yl) piperazine-1-carboxylate

To a solution of 4-chloro-5-iodo-7-tosyl-7H-pyrrolo[2,3-d]pyrimidine (4.0 g, 9.2 mmol, prepared following the procedure outlined in compound 134 in EtOH (50 mL) was added tert-butyl piperazine-1-carboxylate (1.7 g, 9.2 mmol) and DIPEA (5.0 mL, 28 mmol). The resulting mixture was heated to 100° C. overnight. After being cooled down to room temperature, solvent was removed and the residue was purified by flash column chromatography (silica gel, 0˜30%, ethyl acetate in petroleum ether) to afford tert-butyl 4-(5-iodo-7-tosyl-7H-pyrrolo[2,3-d]pyrimidin-4-yl) piperazine-1-carboxylate (4.5 g, 83%) as a white solid. LC/MS ESI (m/z): 584 (M+H)⁺.

### Step 2. tert-Butyl 4-(5-cyclopropyl-7-tosyl-7H-pyrrolo[2,3-d]pyrimidin-4-yl) piperazine-1-carboxylate

To a solution of tert-butyl 4-(5-iodo-7-tosyl-7H-pyrrolo[2,3-d]pyrimidin-4-yl) piperazine-1-carboxylate (2.0 g, 3.4 mmol) in toluene (50 mL) were added cyclopropylboronic acid (0.35 g, 4.1 mmol), K₂CO₃ (9.5 g, 69 mmol) and Pd-118 (0.22 g, 0.34 mmol). The resulting mixture was heated to 80° C. overnight. After being cooled down to room temperature, the solvent was filtered. The filtrate was concentrated and purified by flash column chromatography (silica gel, 0-30%, ethyl acetate in petroleum ether) to afford tert-butyl 4-(5-cyclopropyl-7-tosyl-7H-pyrrolo[2,3-d]pyrimidin-4-yl)piperazine-1-carboxylate (1.3 g, 76%) as a white solid. LC/MS ESI (m/z): 498 (M+H)⁺.

### Step 3. tert-Butyl 4-(5-cyclopropyl-7H-pyrrolo[2,3-d]pyrimidin-4-yl) piperazine-1-carboxylate

To a solution of tert-butyl 4-(5-cyclopropyl-7-tosyl-7H-pyrrolo[2,3-d]pyrimidin-4-yl) piperazine-1-carboxylate (1.3 g, 2.6 mmol) in THE (10 mL) was added TBAF (16 mL, 16 mmol). The resulting mixture was stirred at room temperature overnight. The reaction was quenched with water and extracted with EtOAc twice. The combined organic layers were washed with brine, dried over Na₂SO₄, filtered, and concentrated. The residue was purified by flash column chromatography (silica gel, 0-60%, ethyl acetate in petroleum ether) to afford tert-butyl 4-(5-cyclopropyl-7H-pyrrolo[2,3-d]pyrimidin-4-yl)piperazine-1-carboxylate (810 mg, 90%) as a white solid. LC/MS ESI (m/z): 344 (M+H)⁺.

### Step 4. tert-Butyl 4-(5-cyclopropyl-7-(3,5-difluorophenyl)-7H-pyrrolo[2,3-d]pyrimidin-4-yl) piperazine-1-carboxylate

To a solution of tert-butyl 4-(5-cyclopropyl-7H-pyrrolo[2,3-d]pyrimidin-4-yl)piperazine-1-carboxylate (100 mg, 0.29 mmol) in DMF (5 mL) were added 1,3-difluoro-5-iodobenzene (84 mg, 0.35 mmol), trans-cyclohexane-1,2-diamine (9.9 mg, 0.087 mmol), CuI (17 mg, 0.087 mmol) and K₃PO₄ (190 mg, 0.87 mmol). The resulting mixture was heated to 120° C. overnight. After being cooled down to room temperature. The reaction was quenched with water, extracted with EtOAc twice, the combined organic layers were washed with brine, dried over Na₂SO₄, filtered and concentrated. The residue was purified by flash column chromatography (silica gel, 0-30%, ethyl acetate in petroleum ether) to give crude product which was further purified by prep-HPLC to afford tert-butyl 4-(5-cyclopropyl-7-(3,5-difluorophenyl)-7H-pyrrolo[2,3-d]pyrimidin-4-yl)piperazine-1-carboxylate (82 mg, 62) as a white solid. LC/MS ESI (m/z): 456 (M+H)⁺. ¹H NMR (400 MHz, CDCl₃) δ 8.46 (s, 1H), 7.38-7.33 (m, 2H), 6.91 (d, J=0.8 Hz, 1H), 6.79-6.72 (m, 1H), 3.69 (d, J=2.7 Hz, 4H), 3.65-3.59 (m, 4H), 2.08-2.01 (m, 1H), 1.50 (s, 9H), 1.07-1.00 (m, 2H), 0.80-0.73 (m, 2H).

The following compounds were prepared using a procedure analogous to the synthesis of compound 146 from the corresponding aryl halides, boronic esters or acids and amines. For analogs 474 and 475, trifluoro(oxetan-3-yl)-λ⁴-borane (potassium salt) was used in the coupling step. In case of analogs 465, 466, 467, 468, 469, and 470, the final product was obtained by hydrogenation, similar to Example 69, in the last step.

| Cmpd | | |
|---|---|---|
| No. | Chemical Name | LCMS and ¹H NMR |
| 145 | tert-butyl 4-(7-(4-chloro-3- | LC/MS ESI (m/z): 472 (M + H)⁺. ¹H NMR (400 MHz, |
| | fluorophenyl)-5-cyclopropyl- | CDCl₃) δ 8.45 (s, 1H), 7.63 (dd, J = 10.1, 2.3 Hz, 1H), 7.52- |
| | 7H-pyrrolo[2,3-d]pyrimidin-4- | 7.43 (m, 2H), 6.90 (d, J = 0.8 Hz, 1H), 3.70 (d, J = 2.1 |
| | yl)piperazine-1-carboxylate | Hz, 4H), 3.64-3.60 (m, 4H), 2.08-2.02 (m, 1H), 1.50 (s, |
| | | 9H), 1.05-1.01 (m, 2H), 0.78-0.74 (m, 2H). |
| 149 | tert-butyl 4-(5-cyclopropyl-7-(5- | LC/MS ESI (m/z): 435 (M + H)⁺. ¹H NMR (400 MHz, |
| | methylpyridin-3-yl)-7H- | CDCl₃) δ 8.66 (s, 1H), 8.45 (s, 1H), 8.41 (s, 1H), 7.93 (s, |
| | pyrrolo[2,3-d]pyrimidin-4- | 1H), 6.93 (d, J = 0.6 Hz, 1H), 3.74-3.68 (m, 4H), 3.64- |
| | yl)piperazine-1-carboxylate | 3.61 (m, 4H), 2.45 (s, 3H), 2.09-2.03 (m, 1H), 1.50 (s, |
| | | 9H), 1.07-0.95 (m, 2H), 0.80-0.69 (m, 2H). |
| 464 | tert-butyl 6-(5-cyclopropyl-7- | LC/MS ESI (m/z): 500 (M + H)⁺. ¹H NMR (400 MHz, |
| | (3,5-dichlorophenyl)-7H- | CDCl₃) δ 8.38 (s, 1H), 7.66 (d, J = 1.8 Hz, 2H), 7.30 (t, J = |
| | pyrrolo[2,3-d]pyrimidin-4-yl)- | 1.8 Hz, 1H), 6.92 (d, J = 1.0 Hz, 1H), 4.56 (s, 4H), 4.16 (s, |
| | 2,6-diazaspiro[3.3]heptane-2- | 4H), 1.88-1.83 (m, 1H), 1.46 (s, 9H), 0.99-0.95 (m, 2H), |
| | carboxylate | 0.76-0.72 (m, 2H). |
| 482 | tert-butyl 5-(7-(4-cyanopyridin- | LC/MS ESI (m/z): 474 (M + H)⁺. ¹H NMR(400 MHz, |
| | 2-yl)-5-cyclopropyl-7H- | CDCl₃) δ 9.30 (s, 1H), 8.58 (d, J = 5.0 Hz, 1H), 8.37 (s, |
| | pyrrolo[2,3-d]pyrimidin-4-yl)- | 1H), 7.82 (s, 1H), 7.33 (d, J = 4.8 Hz, 1H), 4.16-4.10 (m, |
| | 1,5-diazocane-1-carboxylate | 2H), 4.05-3.99 (m, 2H), 3.45-3.40 (m, 2H), 3.36-3.31 |
| | | (m, 2H), 2.14-2.07 (m, 4H), 1.93-1.87 (m, 1H), 1.37 (s, |
| | | 9H), 1.01-0.96 (m, 2H), 0.84-0.80 (m, 2H). |
| 465 | tert-butyl 4-(7-(3-chlorophenyl)- | LC/MS ESI (m/z): 498 (M + H)⁺. ¹H NMR(400 MHz, |
| | 5-(tetrahydro-2H-pyran-4-yl)- | CDCl₃) δ 8.52 (s, 1H), 7.74-7.66 (m, 1H), 7.65-7.59 (m, |
| | 7H-pyrrolo[2,3-d]pyrimidin-4- | 1H), 7.49-7.42 (m, 1H), 7.38-7.31 (m, 1H), 7.13 (s, 1H), |
| | yl)piperazine-1-carboxylate | 4.14-4.08 (m, 2H), 3.66-3.55 (m, 6H), 3.50-3.43 (m, |
| | | 4H), 3.10-3.00 (m, 1H), 2.14-2.08 (m, 2H), 1.78-1.67 |
| | | (m, 2H), 1.51 (s, 9H). |
| 466 | tert-butyl 4-(7-(3-chlorophenyl)- | LC/MS ESI (m/z): 512 (M + H)⁺. ¹H NMR(400 MHz, |
| | 5-(tetrahydro-2H-pyran-4-yl)- | CDCl₃) δ 8.53 (s, 1H), 7.73 (t, J = 2.0 Hz, 1H), 7.66-7.62 |
| | 7H-pyrrolo[2,3-d]pyrimidin-4- | (m, 1H), 7.45 (t, J = 8.1 Hz, 1H), 7.35-7.31 (m, 1H), 7.12 |
| | yl)-3-methylpiperazine-1- | (s, 1H), 4.17-4.12 (m, 1H), 4.11-4.05 (m, 2H), 3.87 (s, |
| | carboxylate | 1H), 3.64-3.53 (m, 4H), 3.50-3.33 (m, 3H), 3.08 (t, J = |
| | | 11.5 Hz, 1H), 2.21 (d, J = 12.5 Hz, 1H), 2.05-2.00 (m, |
| | | 1H), 1.93-1.82 (m, 1H), 1.59-1.53 (m, 1H), 1.50 (s, 9H), |
| | | 1.17 (d, J = 6.1 Hz, 3H). |
| 467 | tert-butyl (R)-4-(7-(3,5- | LC/MS ESI (m/z): 514 (M + H)⁺. ¹H NMR(400 MHz, |
| | difluorophenyl)-5-(tetrahydro- | MeOD) δ 8.42 (s, 1H), 7.65-7.57 (m, 2H), 7.54 (s, 1H), |
| | 2H-pyran-4-yl)-7H-pyrrolo[2,3- | 7.01-6.91 (m, 1H), 4.15-4.07 (m, 2H), 4.06-4.00 (m, |
| | d]pyrimidin-4-yl)-3- | 1H), 3.89 (d, J = 13.0 Hz, 1H), 3.69-3.35 (m, 7H), 3.17 (t, |
| | methylpiperazine-1-carboxylate | J = 11.9 Hz, 1H), 2.20 (d, J = 12.1 Hz, 1H), 2.05 (d, J = |
| | | 13.1 Hz, 1H), 1.97-1.87 (m, 1H), 1.65-1.56 (m, 1H), |
| | | 1.49 (s, 9H), 1.13 (t, J = 9.4 Hz, 3H). |
| 468 | tert-butyl (S)-4-(7-(3,5- | LC/MS ESI (m/z): 514 (M + H)⁺. ¹H NMR(400 MHz, |
| | difluorophenyl)-5-(tetrahydro- | MeOD) δ 8.46 (s, 1H), 7.64-7.58 (m, 2H), 7.54 (s, 1H), |
| | 2H-pyran-4-yl)-7H-pyrrolo[2,3- | 7.00-6.92 (m, 1H), 4.14-4.07 (m, 2H), 4.06-4.01 (m, |
| | d]pyrimidin-4-yl)-3- | 1H), 3.89 (d, J = 13.0 Hz, 1H), 3.69-3.36 (m, 7H), 3.17 (t, |
| | methylpiperazine-1-carboxylate | J = 11.8 Hz, 1H), 2.20 (d, J = 13.5 Hz, 1H), 2.05 (d, J = |
| | | 11.9 Hz, 1H), 1.98-1.89 (m, 1H), 1.64-1.55 (m, 1H), |
| | | 1.50 (s, 9H), 1.14 (d, J = 6.5 Hz, 3H). |
| 469 | tert-butyl (R)-4-(7-(3- | LC/MS ESI (m/z): 496 (M + H)⁺. ¹H NMR (400 MHz, |
| | fluorophenyl)-5-(tetrahydro-2H- | MeOD) δ 8.42 (s, 1H), 7.69-7.63 (m, 1H), 7.60-7.51 (m, |
| | pyran-4-yl)-7H-pyrrolo[2,3- | 2H), 7.48 (s, 1H), 7.18-7.07 (m, 1H), 4.14-4.07 (m, 2H), |
| | d]pyrimidin-4-yl)-3- | 4.06-4.01 (m, 1H), 3.89 (d, J = 12.9 Hz, 1H), 3.68-3.49 |
| | methylpiperazine-1-carboxylate | (m, 5H), 3.42-3.34 (m, 2H), 3.18 (t, J = 11.7 Hz, 1H), |
| | | 2.21 (d, J = 13.0 Hz, 1H), 2.11-2.03 (m, 1H), 1.96-1.86 |
| | | (m, 1H), 1.64-1.57 (m, 1H), 1.50 (s, 9H), 1.14 (d, J = 6.5 |
| | | Hz, 3H). |
| 470 | tert-butyl (S)-4-(7-(3- | LC/MS ESI (m/z): 496 (M + H)⁺. ¹H NMR (400 MHz, |
| | fluorophenyl)-5-(tetrahydro-2H- | MeOD) δ 8.42 (s, 1H), 7.68-7.63 (m, 1H), 7.60-7.51 (m, |
| | pyran-4-yl)-7H-pyrrolo[2,3- | 2H), 7.48 (s, 1H), 7.17-7.08 (m, 1H), 4.14-4.07 (m, 2H), |
| | d]pyrimidin-4-yl)-3- | 4.06-4.01 (m, 1H), 3.89 (d, J = 12.9 Hz, 1H), 3.68-3.49 |
| | methylpiperazine-1-carboxylate | (m, 5H), 3.39 (d, J = 13.3 Hz, 2H), 3.22-3.13 (m, 1H), |
| | | 2.21 (d, J = 13.2 Hz, 1H), 2.08-2.03 (m, 1H), 1.97-1.86 |
| | | (m, 1H), 1.64-1.57 (m, 1H), 1.50 (s, 9H), 1.14 (d, J = 6.5 |
| | | Hz, 3H). |
| 471 | tert-butyl (R)-4-(5-(3,6-dihydro- | LC/MS ESI (m/z): 494 (M + H)⁺. ¹H NMR(400 MHz, |
| | 2H-pyran-4-yl)-7-(3- | CDCl₃) δ 8.47 (s, 1H), 7.50 (dd, J = 16.1, 10.0 Hz, 3H), |
| | fluorophenyl)-7H-pyrrolo[2,3- | 7.23 (s, 1H), 7.07 (t, J = 8.1 Hz, 1H), 5.99 (s, 1H), 4.54 (s, |
| | d]pyrimidin-4-yl)-3- | 1H), 4.35 (s, 2H), 4.08-3.69 (m, 5H), 3.43 (t, J = 12.4 Hz, |
| | methylpiperazine-1-carboxylate | 1H), 3.30 (s, 1H), 2.95 (s, 1H), 2.63 (d, J = 16.3 Hz, 1H), |
| | | 2.45 (s, 1H), 1.49 (s, 9H), 1.14 (d, J = 6.5 Hz, 3H). |
| 472 | tert-butyl (S)-4-(5-(3,6-dihydro- | LC/MS ESI (m/z): 494 (M + H)⁺. ¹H NMR(400 MHz, |
| | 2H-pyran-4-yl)-7-(3- | CDCl₃) δ 8.47 (s, 1H), 7.54-7.46 (m, 3H), 7.23 (s, 1H), |
| | fluorophenyl)-7H-pyrrolo[2,3- | 7.10-7.02 (m, 1H), 5.99 (s, 1H), 4.54 (s, 1H), 4.35 (d, J = |
| | d]pyrimidin-4-yl)-3- | 2.5 Hz, 2H), 4.04-3.70 (m, 5H), 3.43 (t, J = 11.3 Hz, 1H), |
| | methylpiperazine-1-carboxylate | 3.30 (s, 1H), 2.93 (s, 1H), 2.62 (d, J = 17.0 Hz, 1H), 2.45 (s, |
| | | 1H), 1.49 (s, 9H), 1.14 (d, J = 6.6 Hz, 3H). |
| 473 | tert-butyl (S)-4-(7-(3,5- | LC/MS ESI (m/z): 512 (M + H)⁺. ¹H NMR (400 MHz, |
| | difluorophenyl)-5-(3,6-dihydro- | CDCl₃) δ 8.47 (s, 1H), 7.40 (d, J = 7.7 Hz, 2H), 7.21 (s, |
| | 2H-pyran-4-yl)-7H-pyrrolo[2,3- | 1H), 6.80 (t, J = 8.8 Hz, 1H), 5.99 (s, 1H), 4.55 (s, 1H), |
| | d]pyrimidin-4-yl)-3- | 4.35 (s, 2H), 4.13-3.70 (m, 5H), 3.42 (t, J = 12.3 Hz, 1H), |
| | methylpiperazine-1-carboxylate | 3.30 (s, 1H), 2.93 (s, 1H), 2.61 (d, J = 16.9 Hz, 1H), 2.42 |
| | | (d, J = 15.3 Hz, 1H), 1.49 (s, 9H), 1.14 (d, J = 6.5 Hz, 3H). |
| 474 | tert-butyl (S)-4-(7-(3- | LC/MS ESI (m/z): 468 (M + H)⁺. ¹H NMR(400 MHz, |
| | fluorophenyl)-5-(oxetan-3-yl)- | CDCl₃) δ 8.41 (s, 1H), 7.47-7.37 (m, 3H), 7.27 (s, 1H), |
| | 7H-pyrrolo[2,3-d]pyrimidin-4- | 7.03-6.97 (m, 1H), 5.42 (d, J = 1.4 Hz, 1H), 5.31 (br s, |
| | yl)-3-methylpiperazine-1- | 1H), 4.48-4.35 (m, 3H), 4.01 (br s, 1H), 3.72 (br s, 1H), |
| | carboxylate | 3.63 (d, J = 13.2 Hz, 1H), 3.42-3.30 (m, 1H), 3.23 (br s, |
| | | 1H), 2.94 (br s, 1H), 2.20 (br s, 1H), 1.41 (s, 9H), 1.06 (d, |
| | | J = 6.6 Hz, 3H). |
| 475 | tert-butyl (S)-4-(7-(3,5- | LC/MS ESI (m/z): 486 (M + H)⁺. ¹H NMR (400 MHz, |
| | difluorophenyl)-5-(oxetan-3-yl)- | CDCl₃) δ 8.48 (s, 1H), 7.42-7.36 (m, 2H), 7.33 (s, 1H), |
| | 7H-pyrrolo[2,3-d]pyrimidin-4- | 6.84-6.76 (m, 1H), 5.50 (br s, 1H), 5.38 (br s, 1H), 4.54- |
| | yl)-3-methylpiperazine-1- | 4.42 (m, 3H), 4.13-3.93 (m, 1H), 3.80 (br s, 1H), 3.70 (d, |
| | carboxylate | J = 13.4 Hz, 1H), 3.48-3.37 (m, 1H), 3.28 (br s, 1H), 3.01 |
| | | (br s, 1H), 2.46 (br s, 1H), 1.48 (s, 9H), 1.13 (d, J = 6.6 Hz, |
| | | 3H). |

### Example 2. Synthesis of tert-butyl (2R,5S)-4-(5-(2-fluorophenyl)-7-(pyridin-2-yl)-7H-pyrrolo[2,3-d]pyrimidin-4-yl)-2,5-dimethylpiperazine-1-carboxylate (Compound 134)

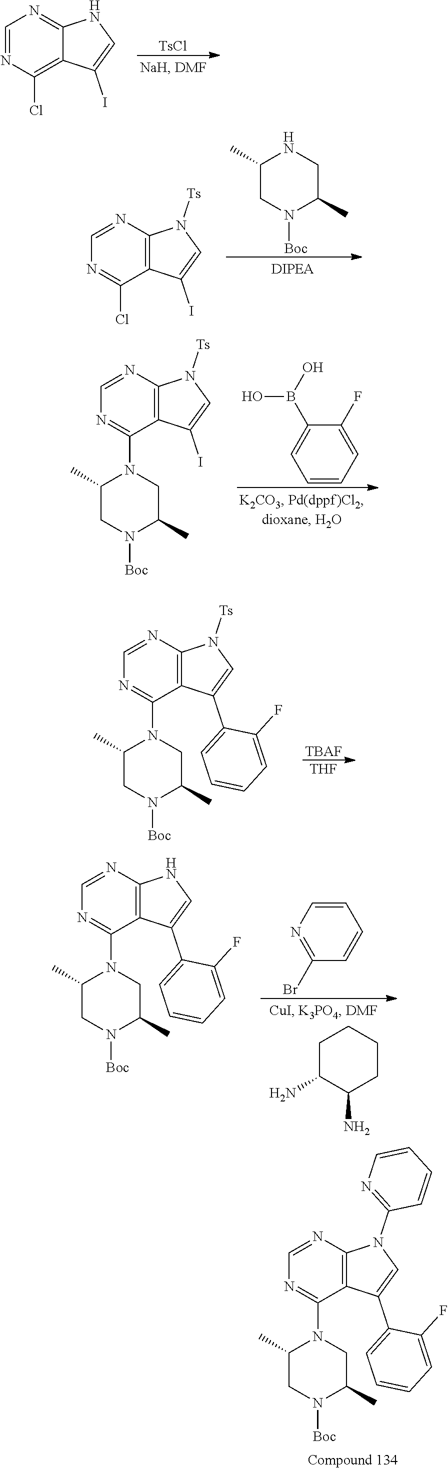

### Step 1. 4-Chloro-5-iodo-7-tosyl-7H-pyrrolo[2,3-d]pyrimidine

To a suspension of NaH (1.0 g, 27 mmol, 60% wt %) in anhydrous DMF (60 mL) at 0° C. was added 4-chloro-5-iodo-7H-pyrrolo[2,3-d]pyrimidine (5.0 g, 18 mmol) in portions. The resulting mixture was stirred at the same temperature for 30 minutes before TsCl (3.4 g, 18 mmol) was added in portions. After addition, the reaction was stirred at room temperature overnight. The reaction was poured into ice water, filtered, the solid was collected and further dried under vacuum to give 4-chloro-5-iodo-7-tosyl-7H-pyrrolo[2,3-d]pyrimidine (6.0 g, 77%) as a white solid. LCMS ESI (m/z): 434 (M+H)⁺.

### Step 2. tert-Butyl (2R,5S)-4-(5-iodo-7-tosyl-7H-pyrrolo[2,3-d]pyrimidin-4-yl)-2,5-dimethylpiperazine-1-carboxylate

To a solution of 4-chloro-5-iodo-7-tosyl-7H-pyrrolo[2,3-d]pyrimidine (4.0 g, 9.2 mmol) in DIPEA (5.0 mL, 28 mmol) was added tert-butyl (2R,5S)-2,5-dimethylpiperazine-1-carboxylate (2.0 g, 9.2 mmol). The resulting mixture was heated to 150° C. for 3 h under N₂. After being cooled down to room temperature, solvent was removed and the residue was purified by flash column chromatography (silica gel, 0-30%, ethyl acetate in petroleum ether) to afford tert-butyl (2R,5S)-4-(5-iodo-7-tosyl-7H-pyrrolo[2,3-d]pyrimidin-4-yl)-2,5-dimethylpiperazine-1-carboxylate (2.5 g, 43%) as a yellow solid. LC/MS ESI (m/z): 612 (M+H)⁺.

### Step 3. tert-Butyl (2R,5S)-4-(5-(2-fluorophenyl)-7-tosyl-7H-pyrrolo[2,3-d]pyrimidin-4-yl)-2,5-dimethylpiperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (2.5 g, 4.0 mmol) in dioxane (30 mL) and H₂O (6 mL) were added (2-fluorophenyl)boronic acid (0.68 g, 4.8 mmol), K₂CO₃ (1.7 g, 12 mmol) and Pd(dppf)Cl₂ (0.29 g, 0.40 mmol). The resulting mixture was stirred at 80° C. overnight under N₂. After being cooled down to room temperature, solvent was removed and the residue was purified by flash column chromatography (silica gel, 0-20%, ethyl acetate in petroleum ether) to afford tert-butyl (2R,5S)-4-(5-(2-fluorophenyl)-7-tosyl-7H-pyrrolo[2,3-d]pyrimidin-4-yl)-2,5-dimethylpiperazine-1-carboxylate (1.9 g, 82%) as a yellow solid. LC/MS ESI (m/z): 580 (M+H)⁺.

### Step 4. tert-Butyl (2R,5S)-4-(5-(2-fluorophenyl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7-tosyl-7H-pyrrolo[2,3-d]pyrimidin-4-yl)-2,5-dimethylpiperazine-1-carboxylate (1.9 g, 3.3 mmol) in THE (20 mL) was added TBAF (20 mL, 20 mmol). The resulting mixture was stirred at room temperature overnight. The reaction was quenched with water and extracted with EtOAc twice. The combined organic layers were washed with brine, dried over Na₂SO₄, filtered, and concentrated. The residue was purified by flash column chromatography (silica gel, 0-60%, ethyl acetate in petroleum ether) to afford tert-butyl (2R,5S)-4-(5-(2-fluorophenyl)-7H-pyrrolo[2,3-d]pyrimidin-4-yl)-2,5-dimethylpiperazine-1-carboxylate (1.2 g, 85%) as a white solid. LC/MS ESI (m/z): 426 (M+H)⁺.

### Step 5. tert-Butyl (2R,5S)-4-(5-(2-fluorophenyl)-7-(pyridin-2-yl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7H-pyrrolo[2,3-d]pyrimidin-4-yl)-2,5-dimethylpiperazine-1-carboxylate (200 mg, 0.47 mmol) in DMF (10 mL) were added 2-bromopyridine (0.05 mL, 0.56 mmol), trans-cyclohexane-1,2-diamine (16 mg, 0.14 mmol), CuI (27 mg, 0.14 mmol) and K₃PO₄ (300 mg, 1.4 mmol). The resulting mixture was heated to 100° C. overnight. After being cooled down to room temperature, the reaction was partitioned between EtOAc and water, the organic layer was separated, and the aqueous layer was extracted with EtOAc twice. The combined organic layers were washed with brine, dried over Na₂SO₄, filtered, and concentrated. The residue was purified by flash column chromatography (silica gel, 0-30%, ethyl acetate in petroleum ether) to afford tert-butyl (2R,5S)-4-(5-(2-fluorophenyl)-7-(pyridin-2-yl)-7H-pyrrolo[2,3-d]pyrimidin-4-yl)-2,5-dimethylpiperazine-1-carboxylate (110 mg, 470%) as a white solid, 30 mg of which was further purified by prep-HPLC to afford 13 mg of white solid. LC/MS ESI (m/z): 503 (M+H)⁺. ¹H NMR (400 Hz, CDCl₃) δ 8.79 (d, J=8.3 Hz, 1H), 8.56 (s, 1H), 8.52-8.48 (m, 1H), 8.26 (s, 1H), 7.93-7.87 (m, 2H), 7.50 (td, J=7.5, 1.6 Hz, 1H), 7.39-7.33 (i, 1H), 7.25-7.17 (m, 3H), 4.30-4.04 (m, 2H), 3.39-3.22 (m, 3H), 2.92-2.70 (m, 1H), 1.43 (s, 9H), 1.08 (d, J=6.8 Hz, 3H), 0.95 (d, J=6.7 Hz, 3H).

The following compounds were prepared by the procedure analogous to the synthesis of compound 134 from the corresponding aryl halides.

| Cmpd | | |
|---|---|---|
| No. | Chemical Name | LCMS and ¹H NMR |
| 135 | tert-butyl (2R,5S)-4-(5-(2- | LC/MS ESI (m/z): 533 (M + H)⁺. ¹H NMR (400 MHz, |
| | fluorophenyl)-7-(5- | CDCl₃) δ 8.56 (d, J = 1.8 Hz, 1H), 8.51 (s, 1H), 8.34 (d, J = |
| | methoxypyridin-3-yl)-7H- | 2.5 Hz, 1H), 7.84 (t, J = 2.3 Hz, 1H), 7.49 (td, J = 7.6, 1.7 |
| | pyrrolo[2,3-d]pyrimidin-4-yl)- | Hz, 1H), 7.45 (s, 1H), 7.41-7.35 (m, 1H), 7.29-7.18 (m, |
| | 2,5-dimethylpiperazine-1- | 2H), 4.36-4.07 (m, 2H), 3.96 (s, 3H), 3.47-3.19 (m, 3H), |
| | carboxylate | 3.01-2.76 (m, 1H), 1.43 (s, 9H), 1.09 (d, J = 6.8 Hz, 3H), |
| | | 0.97 (d, J = 6.6 Hz, 3H). |
| 136 | tert-butyl (2R,5S)-4-(5-(2- | LC/MS ESI (m/z): 517 (M + H)⁺. ¹H NMR (400 MHz, |
| | fluorophenyl)-7-(4- | CDCl₃) δ 8.58 (s, 1H), 8.56 (s, 1H), 8.35 (d, J = 5.0 Hz, |
| | methylpyridin-2-yl)-7H- | 1H), 8.22 (s, 1H), 7.50 (td, J = 7.5, 1.6 Hz, 1H), 7.38-7.31 |
| | pyrrolo[2,3-d]pyrimidin-4-yl)- | (m, 1H), 7.25-7.16 (m, 2H), 7.05 (d, J = 5.0 Hz, 1H), 4.34- |
| | 2,5-dimethylpiperazine-1- | 4.07 (m, 2H), 3.39-3.22 (m, 3H), 2.91-2.70 (m, 1H), |
| | carboxylate | 2.51 (s, 3H), 1.43 (s, 9H), 1.08 (d, J = 6.8 Hz, 3H), 0.94 (d, |
| | | J = 6.7 Hz, 3H). |
| 142 | tert-butyl (2R,5S)-4-(5-(2- | LC/MS ESI (m/z): 503 (M + H)⁺. ¹H NMR (400 MHz, |
| | fluorophenyl)-7-(pyridin-3-yl)- | CDCl₃) δ 8.98 (d, J = 2.4 Hz, 1H), 8.63 (dd, J = 4.8, 1.4 Hz, |
| | 7H-pyrrolo[2,3-d]pyrimidin-4- | 1H), 8.51 (s, 1H), 8.24 (ddd, J = 8.2, 2.6, 1.5 Hz, 1H), 7.52- |
| | yl)-2,5-dimethylpiperazine-1- | 7.47 (m, 2H), 7.46 (d, J = 0.5 Hz, 1H), 7.41-7.34 (m, |
| | carboxylate | 1H), 7.29-7.18 (m, 2H), 4.30-4.00 (m, 2H), 3.44-3.20 |
| | | (m, 3H), 3.01-2.78 (m, 1H), 1.43 (s, 9H), 1.09 (d, J = 6.8 |
| | | Hz, 3H), 0.98 (d, J = 6.7 Hz, 3H). |
| 176 | tert-butyl (2R,5S)-4-(5-(2- | LC/MS ESI (m/z): 506 (M + H)⁺. ¹H NMR (400 MHz, |
| | fluorophenyl)-7-(1-methyl-1H- | CDCl3) δ 8.51 (s, 1H), 8.12 (s, 1H), 7.79 (s, 1H), 7.47 (td, |
| | pyrazol-4-yl)-7H-pyrrolo[2,3- | J = 7.6, 1.7 Hz, 1H), 7.38-7.31 (m, 2H), 7.26-7.17 (m, |
| | d]pyrimidin-4-yl)-2,5- | 2H), 4.36-4.05 (m, 2H), 4.00 (s, 3H), 3.44-3.17 (m, 3H), |
| | dimethylpiperazine-1- | 2.89 (s, 1H), 1.43 (s, 9H), 1.08 (d, J = 6.8 Hz, 3H), 0.95 (d, |
| | carboxylate | J = 6.7 Hz, 3H). |

### Example 3. Synthesis of ethyl (2R,5S)-4-(5-(2-fluorophenyl)-7-(pyridin-2-yl)-7H-pyrrolo[2,3-d]pyrimidin-4-yl)-2,5-dimethylpiperazine-1-carboxylate (Compound 137)

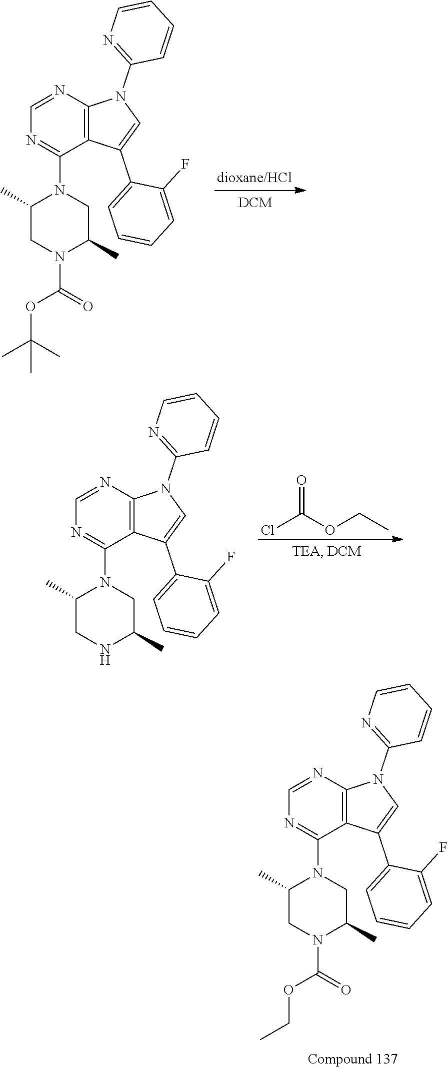

### Step 1. 4-((2S,5R)-2,5-Dimethylpiperazin-1-yl)-5-(2-fluorophenyl)-7-(pyridin-2-yl)-7H-pyrrolo[2,3-d]pyrimidine

To a solution of tert-butyl (2R,5S)-4-(5-(2-fluorophenyl)-7-(pyridin-2-yl)-7H-pyrrolo[2,3-d]pyrimidin-4-yl)-2,5-dimethylpiperazine-1-carboxylate (80 mg, 0.16 mmol) in DCM (3 mL) was added HCl (4.0 mL, 4.0 M in dioxane). The resulting mixture was stirred at room temperature for 4 h. After removal of solvent, the residue was diluted with DCM, washed with NaHCO₃ (aq.), and the organic layer was extracted with DCM twice. The combined organic layers were washed with brine, dried over Na₂SO₄, filtered, and concentrated to provide 4-((2S,5R)-2,5-dimethylpiperazin-1-yl)-5-(2-fluorophenyl)-7-(pyridin-2-yl)-7H-pyrrolo[2,3-d]pyrimidine which was carried directly to the next step. LC/MS ESI (m/z): 403 (M+H)⁺.

### Step 2. Ethyl (2R,5S)-4-(5-(2-fluorophenyl)-7-(pyridin-2-yl)-7H-pyrrolo[2,3-d]pyrimidin-4-yl)-2,5-dimethylpiperazine-1-carboxylate

To a 0° C. solution of 4-((2S,5R)-2,5-dimethylpiperazin-1-yl)-5-(2-fluorophenyl)-7-(pyridin-2-yl)-7H-pyrrolo[2,3-d]pyrimidine (60 mg, 0.15 mmol) in DCM (3 mL) was added TEA (0.062 mL, 0.45 mmol), followed by ethyl chloroformate (0.03 mL, 0.29 mmol) dropwise. The resulting mixture was stirred at room temperature overnight. The reaction was quenched with water, extracted with DCM twice and the combined organic layers were washed with NaHCO₃ (aq.), dried over Na₂SO₄, filtered, and concentrated. The residue was purified by flash column chromatography (silica gel, 0-0, ethyl acetate in petroleum ether) to give the product which was further purified by prep-HPLC to afford ethyl (2R,5s)-4-(5-(2-fluorophenyl)-7-(pyridin-2-yl)-7H-pyrrolo[2,3-d]pyrimidin-4-yl)-2,5-dimethylpiperazine-1-carboxylate (39 mg, 54%) as a white solid. LC/MS ESI (m/z): 475 (M+H)⁺. ¹H NMR (400 Hz, CDC D₃) δ 8.79 (d, J=8.3 Hz, 1H), 8.56 (s, 1H), 8.52-8.48 (m, 1H), 8.26 (s, 1H), 7.93-7.87 (m, 1H), 7.50 (td, J=7.5, 1.6 Hz, 1H), 7.40-7.31 (i, 1H), 7.25-7.15 (m, 3H), 4.37-4.04 (m, 4H), 3.43-3.22 (m, 3H), 2.95-2.71 (m, 1H), 1.23 (t, J=6.5 Hz, 3H), 1.12 (d, J=6.8 Hz, 3H), 0.95 (d, J=6.7 Hz, 3H).

The following compounds were prepared using a procedure analogous to the synthesis of compound 137 from the corresponding aryl halides.

| Cmpd | | |
|---|---|---|
| No. | Chemical Name | LCMS and ¹H NMR |
| 138 | ethyl (2R,5S)-4-(5-(2- | LC/MS ESI (m/z): 505 (M + H)⁺. ¹H NMR (400 MHz, |
| | fluorophenyl)-7-(5- | CDCl₃) δ 8.56 (d, J = 1.6 Hz, 1H), 8.51 (s, 1H), 8.34 (d, J = |
| | methoxypyridin-3-yl)-7H- | 2.4 Hz, 1H), 7.84 (t, J = 2.3 Hz, 1H), 7.49 (td, J = 7.6, 1.6 |
| | pyrrolo[2,3-d]pyrimidin-4-yl)- | Hz, 1H), 7.45 (s, 1H), 7.42-7.34 (m, 1H), 7.29-7.18 (m, |
| | 2,5-dimethylpiperazine-1- | 2H), 4.35-4.07 (m, 4H), 3.96 (s, 3H), 3.48-3.16 (m, 3H), |
| | carboxylate | 3.04-2.70 (m, 1H), 1.23 (t, J = 7.0 Hz, 3H), 1.12 (d, J = |
| | | 6.8 Hz, 3H), 0.97 (d, J = 6.7 Hz, 3H). |
| 143 | ethyl (2R,5S)-4-(5-(2- | LC/MS ESI (m/z): 475 (M + H)⁺. ¹H NMR (400 MHz, |
| | fluorophenyl)-7-(pyridin-3-yl)- | CDCl₃) δ 8.98 (d, J = 2.1 Hz, 1H), 8.64 (d, J = 3.9 Hz, 1H), |
| | 7H-pyrrolo[2,3-d]pyrimidin-4- | 8.52 (s, 1H), 8.24 (ddd, J = 8.2, 2.5, 1.5 Hz, 1H), 7.53- |
| | yl)-2,5-dimethylpiperazine-1- | 7.47 (m, 2H), 7.46 (s, 1H), 7.41-7.35 (m, 1H), 7.29-7.19 |
| | carboxylate | (m, 2H), 4.34-4.02 (m, 4H), 3.47-3.23 (m, 3H), 3.01- |
| | | 2.77 (m, 1H), 1.23 (t, J = 6.7 Hz, 3H), 1.12 (d, J = 6.8 Hz, |
| | | 3H), 0.97 (d, J = 6.7 Hz, 3H). |
| 144 | ethyl (2R,5S)-4-(5-(2- | LC/MS ESI (m/z): 489 (M + H)⁺. ¹H NMR (400 MHz, |
| | fluorophenyl)-7-(4- | CDCl₃) δ 8.58 (s, 1H), 8.56 (s, 1H), 8.35 (d, J = 5.0 Hz, |
| | methylpyridin-2-yl)-7H- | 1H), 8.22 (s, 1H), 7.50 (td, J = 7.6, 1.7 Hz, 1H), 7.38-7.32 |
| | pyrrolo[2,3-d]pyrimidin-4-yl)- | (m, 1H), 7.25-7.16 (m, 2H), 7.05 (dd, J = 5.0, 0.6 Hz, |
| | 2,5-dimethylpiperazine-1- | 1H), 4.32-4.07 (m, 4H), 3.42-3.25 (m, 3H), 2.93-2.77 |
| | carboxylate | (m, 1H), 2.51 (s, 3H), 1.23 (t, J = 6.4 Hz, 3H), 1.12 (d, J = |
| | | 6.8 Hz, 3H), 0.94 (d, J = 6.7 Hz, 3H). |

### Example 4. Synthesis of tert-butyl 4-(7-(5-chloro-2-methylphenyl)-5-(pyridin-2-yl)-7H-pyrrolo[2,3-d]pyrimidin-4-yl)-3-methylpiperazine-1-carboxylate (Compound 159)

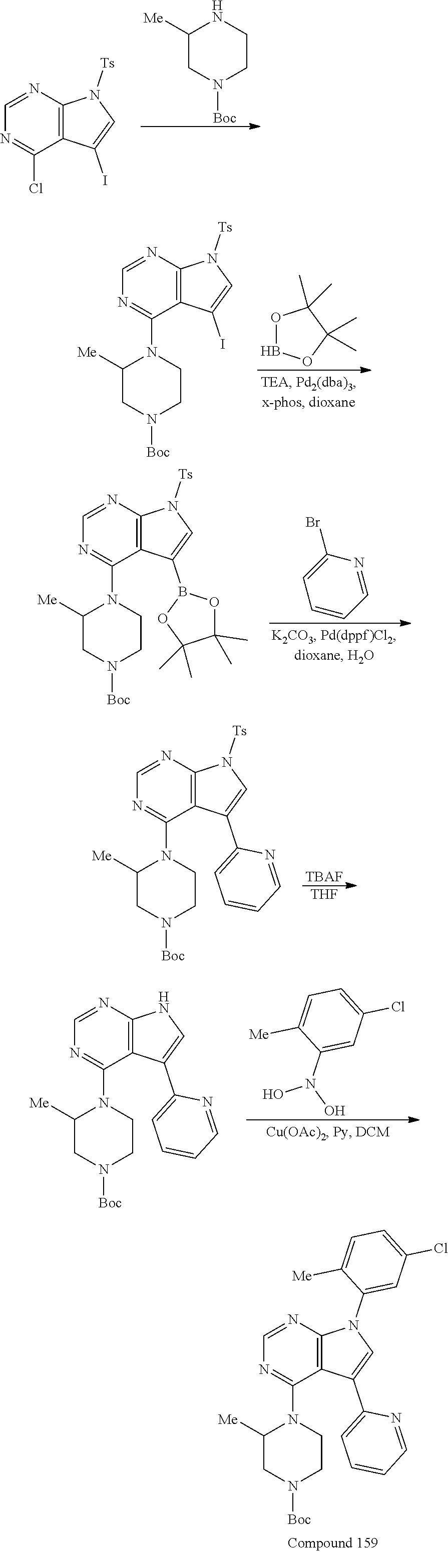

### Step 1. tert-Butyl 4-(5-iodo-7-tosyl-7H-pyrrolo[2,3-d]pyrimidin-4-yl)-3-methylpiperazine-1-carboxylate

The mixture of 4-chloro-5-iodo-7-tosyl-7H-pyrrolo[2,3-d]pyrimidine (3.0 g, 6.9 mmol, prepared following the procedure outlined in compound 134, step 1) and tert-butyl 3-methylpiperazine-1-carboxylate (5.6 g, 28 mmol) was heated to 150° C. for 3 h. After being cooled down to room temperature, the reaction was purified by flash column chromatography (silica gel, 0˜30%, ethyl acetate in petroleum ether) to afford tert-butyl 4-(5-iodo-7-tosyl-7H-pyrrolo[2,3-d]pyrimidin-4-yl)-3-methylpiperazine-1-carboxylate (2.0 g, 48%) as a white solid. LC/MS ESI (m/z): 598 (M+H)⁺.

### Step 2. tert-Butyl 3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate

To a solution of tert-butyl 4-(5-iodo-7-tosyl-7H-pyrrolo[2,3-d]pyrimidin-4-yl)-3-methylpiperazine-1-carboxylate (2.0 g, 3.3 mmol) in dioxane (30 mL) were added 4,4,5,5-tetramethyl-1,3,2-dioxaborolane (1.9 mL, 13 mmol), TEA (2.3 mL, 17 mmol), X-Phos (0.16 g, 0.33 mmol) and Pd₂(dba)₃ (0.31 g, 0.33 mmol). The resulting mixture was stirred at 95° C. overnight. After being cooled down to room temperature, the reaction was quenched with water, extracted with DCM twice, the combined organic layers were washed with brine, dried over Na₂SO₄, filtered and concentrated to afford tert-butyl 3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate as a yellow solid which was used in the next step directly. LC/MS ESI (m/z): 598 (M+H)⁺.

### Step 3. tert-Butyl 3-methyl-4-(5-(pyridin-2-yl)-7-tosyl-7H-pyrrolo[2,3-d]pyrimidin-4-yl)piperazine-1-carboxylate

To a solution of tert-butyl 3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate (700 mg, 1.1 mmol) in dioxane (10 mL) and H₂O (2 mL) were added 2-bromopyridine (0.22 mL, 2.3 mmol), K₂CO₃ (810 mg, 5.8 mmol) and Pd(dppf)Cl₂ (86 mg, 0.11 mmol). The resulting mixture was heated to 90° C. overnight. After being cooled down to room temperature, solvent was removed and the residue was purified by flash column chromatography (silica gel, 0-60%, ethyl acetate in petroleum ether) to afford tert-butyl 3-methyl-4-(5-(pyridin-2-yl)-7-tosyl-7H-pyrrolo[2,3-d]pyrimidin-4-yl)piperazine-1-carboxylate (490 mg, 76%) as a yellow solid. LC/MS ESI (m/z): 549 (M+H)⁺.

### Step 4. tert-Butyl 3-methyl-4-(5-(pyridin-2-yl)-7H-pyrrolo[2,3-d]pyrimidin-4-yl)piperazine-1-carboxylate

To a solution of tert-butyl 3-methyl-4-(5-(pyridin-2-yl)-7-tosyl-7H-pyrrolo[2,3-d]pyrimidin-4-yl)piperazine-1-carboxylate (490 mg, 0.89 mmol) in THE (5 mL) was added TBAF (5.4 mL, 5.3 mmol). The resulting mixture was stirred at room temperature overnight. The reaction was quenched with water and extracted with EtOAc twice. The combined organic layers were washed with brine, dried over Na₂SO₄, filtered, and concentrated. The residue was purified by flash column chromatography (silica gel, 0˜10%, methanol in dichloromethane) to afford tert-butyl 3-methyl-4-(5-(pyridin-2-yl)-7H-pyrrolo[2,3-d]pyrimidin-4-yl)piperazine-1-carboxylate (200 mg, 56%) as a white solid. LC/MS ESI (m/z): 395 (M+H)⁺.

### Step 5. tert-Butyl 4-(7-(5-chloro-2-methylphenyl)-5-(pyridin-2-yl)-7H-pyrrolo[2,3-d]pyrimidin-4-yl)-3-methylpiperazine-1-carboxylate

To a solution of tert-butyl 3-methyl-4-(5-(pyridin-2-yl)-7H-pyrrolo[2,3-d]pyrimidin-4-yl)piperazine-1-carboxylate (100 mg, 0.25 mmol) in DCM (10 mL) were added (5-chloro-2-methylphenyl)boronic acid (130 mg, 0.76 mmol), Cu(OAc)₂ (180 mg, 1.0 mmol), pyridine (0.12 mL, 1.5 mmol) and 4 A molecular sieves (400 mg). The resulting mixture was heated to 40° C. for 3 days under an O₂ atmosphere. After being cooled down to room temperature, the reaction was quenched with NH₄OH, diluted with DCM, and then filtered. The filtrate was extracted with DCM twice and the combined organic layers were washed with brine, dried over Na₂SO₄, filtered and concentrated. The residue was purified by flash column chromatography (silica gel, 0-0, ethyl acetate in petroleum ether) to give the product which was further purified by prep-HPLC to afford tert-butyl 4-(7-(5-chloro-2-methylphenyl)-5-(pyridin-2-yl)-7H-pyrrolo[2,3-d]pyrimidin-4-yl)-3-methylpiperazine-1l-carboxylate (30 mg, 22%) as a yellow solid. LC/MS ESI (m/z): 519 (M+H)⁺. ¹H NMR (400 MHz, CDCl₃) δ 8.70-8.65 (m, 1H), 8.47 (s, 1H), 7.78 (td, J=7.7, 1.7 Hz, 1H), 7.61 (d, J=7.7 Hz, 1H), 7.46 (s, 1H), 7.39-7.31 (m, 3H), 7.26-7.23 (m, 1H), 4.48-4.19 (m, 1H), 3.96-3.78 (m, 1H), 3.68-3.48 (m, 2H), 3.23-2.72 (m, 3H), 2.11 (s, 3H), 1.44 (s, 9H), 1.07 (s, 3H).

The following compounds were prepared using a procedure analogous to the synthesis of compound 159 from the corresponding aryl halides.

| Cmpd | | |
|---|---|---|
| No. | Chemical Name | LCMS and ¹H NMR |
| 160 | tert-butyl 4-(7-(3-chloro-2- | LC/MS ESI (m/z): 519 (M + H)⁺. ¹H NMR (400 MHz, |
| | methylphenyl)-5-(pyridin-2-yl)- | CDCl₃) δ 8.72-8.66 (m, 1H), 8.47 (s, 1H), 7.79 (td, J = |
| | 7H-pyrrolo[2,3-d]pyrimidin-4- | 7.7, 1.8 Hz, 1H), 7.62 (d, J = 7.4 Hz, 1H), 7.51 (dd, J = 7.1, |
| | yl)-3-methylpiperazine-1- | 2.2 Hz, 1H), 7.46 (s, 1H), 7.29 (d, J = 7.9 Hz, 2H), 7.26- |
| | carboxylate | 7.21 (m, 1H), 4.45-4.16 (m, 1H), 3.95-3.77 (m, 1H), |
| | | 3.68-3.49 (m, 2H), 3.23-2.70 (m, 3H), 2.13 (s, 3H), 1.44 |
| | | (s, 9H), 1.07 (s, 3H). |
| 161 | tert-butyl 4-(7-(3-chlorophenyl)- | LC/MS ESI (m/z): 520 (M + H)⁺. ¹H NMR (400 MHz, |
| | 5-(3-methylpyrazin-2-yl)-7H- | CDCl₃) δ 8.53 (d, J = 5.0 Hz, 3H), 7.80 (t, J = 1.9 Hz, 1H), |
| | pyrrolo[2,3-d]pyrimidin-4-yl)-3- | 7.69 (dd, J = 8.0, 0.9 Hz, 1H), 7.48 (dd, J = 10.1, 6.0 Hz, |
| | methylpiperazine-1-carboxylate | 2H), 7.41-7.36 (m, 1H), 4.23-3.92 (m, 1H), 3.82-3.64 |
| | | (m, 1H), 3.58-3.38 (m, 2H), 3.14-2.94 (m, 1H), 2.89- |
| | | 2.68 (m, 1H), 2.65-2.47 (m, 4H), 1.43 (s, 9H), 0.98 (s, |
| | | 3H). |
| 162 | tert-butyl 4-(7-(3-chloro-2- | LC/MS ESI (m/z): 520 (M + H)⁺. ¹H NMR (400 MHz, |
| | methylphenyl)-5-(pyrazin-2-yl)- | CDCl₃) δ 8.95 (s, 1H), 8.62 (s, 1H), 8.52 (d, J = 4.9 Hz, |
| | 7H-pyrrolo[2,3-d]pyrimidin-4- | 2H), 7.60-7.49 (m, 2H), 7.35-7.26 (m, 2H), 4.43-4.13 |
| | yl)-3-methylpiperazine-1- | (m, 1H), 3.96-3.77 (m, 1H), 3.66-3.45 (m, 2H), 3.31- |
| | carboxylate | 2.75 (m, 3H), 2.13 (s, 3H), 1.44 (s, 9H), 1.12 (s, 3H). |
| 163 | tert-butyl 4-(7-(5-chloro-2- | LC/MS ESI (m/z): 520 (M + H)⁺. ¹H NMR (400 MHz, |
| | methylphenyl)-5-(pyrazin-2-yl)- | CDCl₃) δ 8.94 (s, 1H), 8.66-8.59 (m, 1H), 8.52 (d, J = 3.1 |
| | 7H-pyrrolo[2,3-d]pyrimidin-4- | Hz, 2H), 7.58 (s, 1H), 7.40 (dd, J = 8.2, 2.2 Hz, 1H), 7.37- |
| | yl)-3-methylpiperazine-1- | 7.32 (m, 2H), 4.26 (d, J = 52.2 Hz, 1H), 4.00-3.74 (m, |
| | carboxylate | 1H), 3.70-3.39 (m, 2H), 3.34-2.68 (m, 3H), 2.12 (s, 3H), |
| | | 1.44 (s, 9H), 1.09 (d, J = 18.6 Hz, 3H). |

### Example 5. Synthesis of tert-butyl (S)-4-(7-(3-cyanophenyl)-5-(pyridin-2-yl)-7H-pyrrolo[2,3-d]pyrimidin-4-yl)-3-methylpiperazine-1-carboxylate (Compound 280)

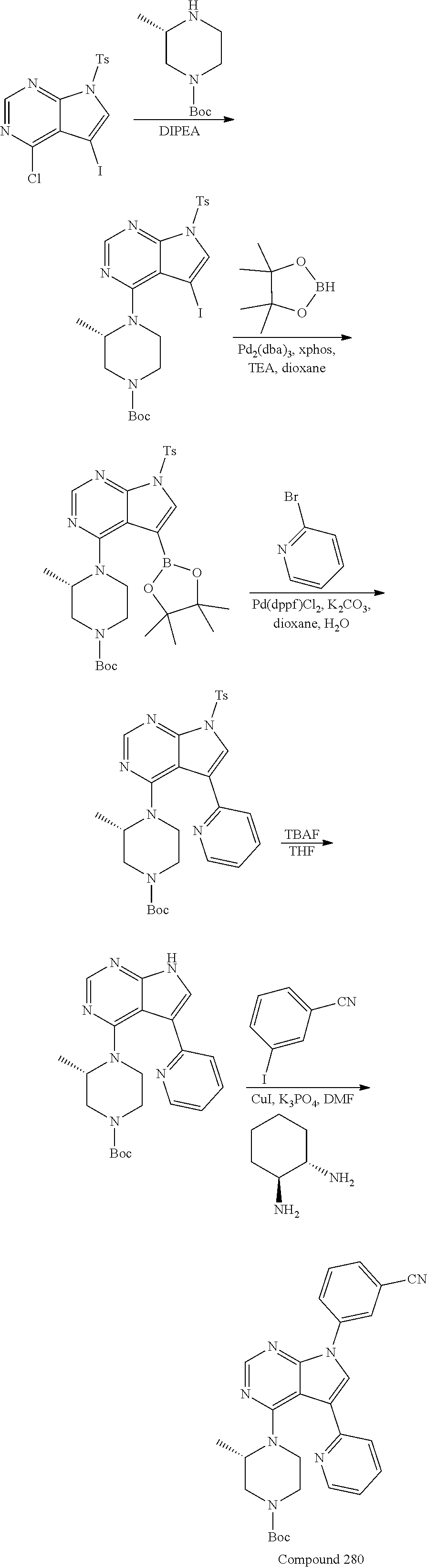

### Step 1. tert-Butyl (S)-4-(5-iodo-7-tosyl-7H-pyrrolo[2,3-d]pyrimidin-4-yl)-3-methylpiperazine-1-carboxylate

To a solution of 4-chloro-5-iodo-7-tosyl-7H-pyrrolo[2,3-d]pyrimidine (5.0 g, 12 mmol) in DIPEA (15 mL) was added tert-butyl (S)-3-methylpiperazine-1-carboxylate (5.8 g, 29 mmol). The resulting mixture was heated to 140° C. for 1.5 h. After being cooled down to room temperature, solvent was removed and the residue was purified by flash column chromatography (silica gel, 0˜20%, ethyl acetate in petroleum ether) to afford tert-butyl (S)-4-(5-iodo-7-tosyl-7H-pyrrolo[2,3-d]pyrimidin-4-yl)-3-methylpiperazine-1-carboxylate (5.8 g, 84%) as a yellow solid. LC/MS ESI (m/z): 598 (M+H)⁺.

### Step 2. tert-Butyl (S)-3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (1.0 g, 1.6 mmol) in dioxane (15 mL) were added 4,4,5,5-tetramethyl-1,3,2-dioxaborolane (0.97 mL, 6.6 mmol), TEA (1.2 mL, 8.3 mmol), X-Phos (0.08 g, 0.16 mmol) and Pd₂(dba)₃ (0.15 g, 0.16 mmol). The resulting mixture was stirred at 95° C. overnight. After being cooled down to room temperature. The reaction was quenched with water, extracted with DCM twice, the combined organic layers were washed with brine, dried over Na₂SO₄, filtered and concentrated to afford crude tert-butyl (S)-3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate (1.0 g, 99%) as a yellow oil. LC/MS ESI (m/z): 598 (M+H)⁺.

### Step 3. tert-Butyl (S)-3-methyl-4-(5-(pyridin-2-yl)-7-tosyl-7H-pyrrolo[2,3-d]pyrimidin-4-yl)piperazine-1-carboxylate

To a solution of tert-butyl (S)-3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate (1.0 g, 1.6 mmol) in dioxane (15 mL) and H₂O (3 mL) were added 2-bromopyridine (0.32 mL, 3.3 mmol), K₂CO₃ (1.2 g, 8.3 mmol) and Pd(dppf)Cl₂ (0.12 g, 0.16 mmol). The resulting mixture was heated to 90° C. overnight. After being cooled down to room temperature, the solvent was removed and the residue was purified by flash column chromatography (silica gel, 0˜50%, ethyl acetate in petroleum ether) to afford tert-butyl (S)-3-methyl-4-(5-(pyridin-2-yl)-7-tosyl-7H-pyrrolo[2,3-d]pyrimidin-4-yl)piperazine-1-carboxylate (0.69 g, 75%) as a yellow solid. LC/MS ESI (m/z): 549 (M+H)⁺.

### Step 4. tert-Butyl (S)-3-methyl-4-(5-(pyridin-2-yl)-7H-pyrrolo[2,3-d]pyrimidin-4-yl)piperazine-1-carboxylate

To a solution of tert-butyl (S)-3-methyl-4-(5-(pyridin-2-yl)-7-tosyl-7H-pyrrolo[2,3-d]pyrimidin-4-yl)piperazine-1-carboxylate (690 mg, 1.3 mmol) in THE (5 mL) was added TBAF (5.0 mL, 5.0 mmol, 1.0M in THF). The resulting mixture was stirred at room temperature overnight. The reaction was quenched with water and extracted with EtOAc twice. The combined organic layers were washed with brine, dried over Na₂SO₄, filtered and concentrated. The residue was purified by flash column chromatography (silica gel, 0˜10%, methanol in dichloromethane) to afford tert-butyl (S)-3-methyl-4-(5-(pyridin-2-yl)-7H-pyrrolo[2,3-d]pyrimidin-4-yl)piperazine-1-carboxylate (450 mg, 90%) as a white solid. LC/MS ESI (m/z): 395 (M+H)⁺.

### Step 5. tert-Butyl (S)-4-(7-(3-cyanophenyl)-5-(pyridin-2-yl)-7H-pyrrolo[2,3-d]pyrimidin-4-yl)-3-methylpiperazine-1-carboxylate

To a solution of tert-butyl (S)-3-methyl-4-(5-(pyridin-2-yl)-7H-pyrrolo[2,3-d]pyrimidin-4-yl)piperazine-1-carboxylate (100 mg, 0.25 mmol) in DMF (5 mL) were added 3-iodobenzonitrile (87 mg, 0.38 mmol), (1S,2S)-cyclohexane-1,2-diamine (8.6 mg, 0.076 mmol), CuI (48 mg, 0.25 mmol) and K₃PO₄ (160 mg, 0.76 mmol). The resulting mixture was heated to 120° C. overnight. After being cooled down to room temperature. The reaction was quenched with water, extracted with EtOAc twice, the combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford tert-butyl (S)-4-(7-(3-cyanophenyl)-5-(pyridin-2-yl)-7H-pyrrolo[2,3-d]pyrimidin-4-yl)-3-methylpiperazine-1-carboxylate (52 mg, 410) as a yellow solid. LC/MS ESI (m/z): 496 (M+H). ¹H NMR (400 MHz, DMSO-d6) δ 8.69 (dd, J=4.8, 0.8 Hz, 1H), 8.45 (s, 1H), 8.45-8.43 (s, 1H), 8.34-8.27 (m, 1H), 8.17 (s, 1H), 7.95 (td, J=7.7, 1.8 Hz, 1H), 7.86 (dt, J=7.7, 1.2 Hz, 1H), 7.77 (t, J=8.0 Hz, 1H), 7.69 (d, J=7.9 Hz, 1H), 7.41-7.35 (m, 1H), 4.21 (d, J=6.4 Hz, 1H), 3.77-3.60 (m, 2H), 3.12-22.55 (m, 4H), 1.36 (s, 9H), 0.92 (d, J=6.6 Hz, 3H).

The following compounds were prepared using a procedure analogous to the synthesis of compound 280 from the corresponding aryl halides.

| Cmpd | | |
|---|---|---|
| No. | Chemical Name | LCMS and ¹H NMR |
| 293 | tert-butyl (S)-4-(7-(3- | LC/MS ESI (m/z): 516 (M + H)⁺. ¹H NMR (400 MHz, |
| | methoxyphenyl)-5-(3- | MeOD) δ 8.58 (dd, J = 7.6, 2.4 Hz, 2H), 8.42 (s, 1H), 7.79 |
| | methylpyrazin-2-yl)-7H- | (s, 1H), 7.47 (t, J = 8.2 Hz, 1H), 7.37 (t, J = 2.2 Hz, 1H), |
| | pyrrolo[2,3-d]pyrimidin-4-yl)-3- | 7.33-7.27 (m, 1H), 7.02 (dd, J = 8.0, 2.1 Hz, 1H), 4.03 (s, |
| | methylpiperazine-1-carboxylate | 1H), 3.88 (s, 3H), 3.74 (d, J = 12.5 Hz, 1H), 3.58-3.45 (m, |
| | | 2H), 3.07 (td, J = 13.0, 2.9 Hz, 1H), 2.85-2.57 (m, 2H), |
| | | 2.56 (s, 3H), 1.42 (s, 9H), 0.93 (d, J = 6.4 Hz, 3H). |
| 294 | tert-butyl (S)-4-(7-(3- | LC/MS ESI (m/z): 511 (M + H)⁺. ¹H NMR (400 MHz, |
| | cyanophenyl)-5-(3- | MeOD) δ 8.60 (d, J = 2.5 Hz, 1H), 8.58 (d, J = 2.6 Hz, 1H), |
| | methylpyrazin-2-yl)-7H- | 8.47 (s, 1H), 8.34-8.31 (m, 1H), 8.18-8.14 (m, 1H), 7.90 |
| | pyrrolo[2,3-d]pyrimidin-4-yl)-3- | (s, 1H), 7.79 (dt, J = 7.7, 1.4 Hz, 1H), 7.75 (t, J = 7.8 Hz, |
| | methylpiperazine-1-carboxylate | 1H), 4.05 (s, 1H), 3.74 (d, J = 13.2 Hz, 1H), 3.51 (t, J = |
| | | 12.8 Hz, 2H), 3.06 (td, J = 13.0, 3.0 Hz, 1H), 2.83-2.58 |
| | | (m, 2H), 2.56 (s, 3H), 1.42 (s, 9H), 0.94 (d, J = 6.4 Hz, 3H). |
| 295 | tert-butyl (S)-4-(7-(3- | LC/MS ESI (m/z): 502 (M + H)⁺. ¹H NMR (400 MHz, |
| | methoxyphenyl)-5-(pyrazin-2- | MeOD) δ 8.97 (d, J = 1.4 Hz, 1H), 8.70 (dd, J = 2.5, 1.5 |
| | yl)-7H-pyrrolo[2,3-d]pyrimidin- | Hz, 1H), 8.55 (d, J = 2.6 Hz, 1H), 8.43 (s, 1H), 8.02 (s, 1H), |
| | 4-yl)-3-methylpiperazine-1- | 7.47 (t, J = 8.2 Hz, 1H), 7.36 (t, J = 2.2 Hz, 1H), 7.32- |
| | carboxylate | 7.27 (m, 1H), 7.06-6.99 (m, 1H), 4.26-4.14 (m, 1H), |
| | | 3.92-3.80 (m, 4H), 3.61 (d, J = 13.2 Hz, 1H), 3.54 (d, J = |
| | | 13.3 Hz, 1H), 3.25-3.14 (m, 1H), 3.08-2.76 (m, 2H), |
| | | 1.43 (s, 9H), 1.03 (d, J = 6.5 Hz, 3H). |
| 296 | tert-butyl (S)-3-methyl-4-(5- | LC/MS ESI (m/z): 556 (M + H)⁺. ¹H NMR (400 MHz, |
| | (pyrazin-2-yl)-7-(3- | MeOD) δ 8.99 (s, 1H), 8.72 (dd, J = 2.4, 1.6 Hz, 1H), 8.57 |
| | (trifluoromethoxy)phenyl)-7H- | (d, J = 2.5 Hz, 1H), 8.47 (s, 1H), 8.10 (s, 1H), 7.91 (s, 1H), |
| | pyrrolo[2,3-d]pyrimidin-4- | 7.83 (dd, J = 8.1, 1.4 Hz, 1H), 7.68 (t, J = 8.2 Hz, 1H), 7.41- |
| | yl)piperazine-1-carboxylate | 7.35 (m, 1H), 4.26-4.13 (m, 1H), 3.85 (d, J = 13.8 Hz, |
| | | 1H), 3.62 (d, J = 13.1 Hz, 1H), 3.53 (d, J = 13.4 Hz, 1H), |
| | | 3.19 (td, J = 12.9, 3.4 Hz, 1H), 3.07-2.80 (m, 2H), 1.43 (s, |
| | | 9H), 1.03 (d, J = 6.5 Hz, 3H). |
| 297 | tert-butyl (S)-4-(7-(3- | LC/MS ESI (m/z): 497 (M + H)⁺. ¹H NMR (400 MHz, |
| | cyanophenyl)-5-(pyrazin-2-yl)- | MeOD) δ 8.98 (s, 1H), 8.72 (dd, J = 2.4, 1.6 Hz, 1H), 8.58 |
| | 7H-pyrrolo[2,3-d]pyrimidin-4- | (d, J = 2.5 Hz, 1H), 8.48 (s, 1H), 8.34-8.31 (m, 1H), 8.18- |
| | yl)-3-methylpiperazine-1- | 8.15 (m, 1H), 8.14 (s, 1H), 7.80 (dt, J = 7.7, 1.4 Hz, 1H), |
| | carboxylate | 7.76 (t, J = 7.8 Hz, 1H), 4.26-4.17 (m, 1H), 3.85 (d, J = |
| | | 12.9 Hz, 1H), 3.62 (d, J = 13.2 Hz, 1H), 3.57-3.50 (m, |
| | | 1H), 3.19 (td, J = 12.9, 3.4 Hz, 1H), 3.07-2.79 (m, 2H), |
| | | 1.43 (s, 9H), 1.04 (d, J = 6.5 Hz, 3H). |
| 304 | tert-butyl (S)-3-methyl-4-(5-(3- | LC/MS ESI (m/z): 570 (M + H)⁺. ¹H NMR (400 MHz, |
| | methylpyrazin-2-yl)-7-(3- | MeOD) δ 8.60 (d, J = 2.6 Hz, 1H), 8.58 (d, J = 2.6 Hz, 1H), |
| | (trifluoromethoxy)phenyl)-7H- | 8.46 (s, 1H), 7.91 (s, 1H), 7.87 (s, 1H), 7.85-7.79 (m, 1H), |
| | pyrrolo[2,3-d]pyrimidin-4- | 7.67 (t, J = 8.2 Hz, 1H), 7.41-7.33 (m, 1H), 4.04 (s, 1H), |
| | yl)piperazine-1-carboxylate | 3.74 (d, J = 13.1 Hz, 1H), 3.54-3.46 (m, 2H), 3.11-3.01 |
| | | (m, 1H), 2.84-2.58 (m, 2H), 2.56 (s, 3H), 1.42 (s, 9H), |
| | | 0.93 (d, J = 6.4 Hz, 3H). |
| 305 | tert-butyl (S)-3-methyl-4-(5-(3- | LC/MS ESI (m/z): 540 (M + H)⁺. ¹H NMR (400 MHz, |
| | methylpyrazin-2-yl)-7-(3,4,5- | MeOD) δ 8.60 (d, J = 2.6 Hz, 1H), 8.58 (d, J = 2.6 Hz, 1H), |
| | trifluorophenyl)-7H-pyrrolo[2,3- | 8.47 (s, 1H), 7.86 (s, 1H), 7.82 (dd, J = 9.0, 6.2 Hz, 2H), |
| | d]pyrimidin-4-yl)piperazine-1- | 4.04 (s, 1H), 3.73 (d, J = 13.2 Hz, 1H), 3.50 (t, J = 14.5 Hz, |
| | carboxylate | 2H), 3.11-3.00 (m, 1H), 2.81-2.60 (m, 2H), 2.54 (s, 3H), |
| | | 1.42 (s, 9H), 0.93 (d, J = 6.3 Hz, 3H). |

### Example 6. Synthesis of tert-butyl (R)-4-(5-(2-fluorophenyl)-7-(pyridin-2-yl)-7H-pyrrolo[2,3-d]pyrimidin-4-yl)-2-methylpiperazine-1-carboxylate (Compound 192)

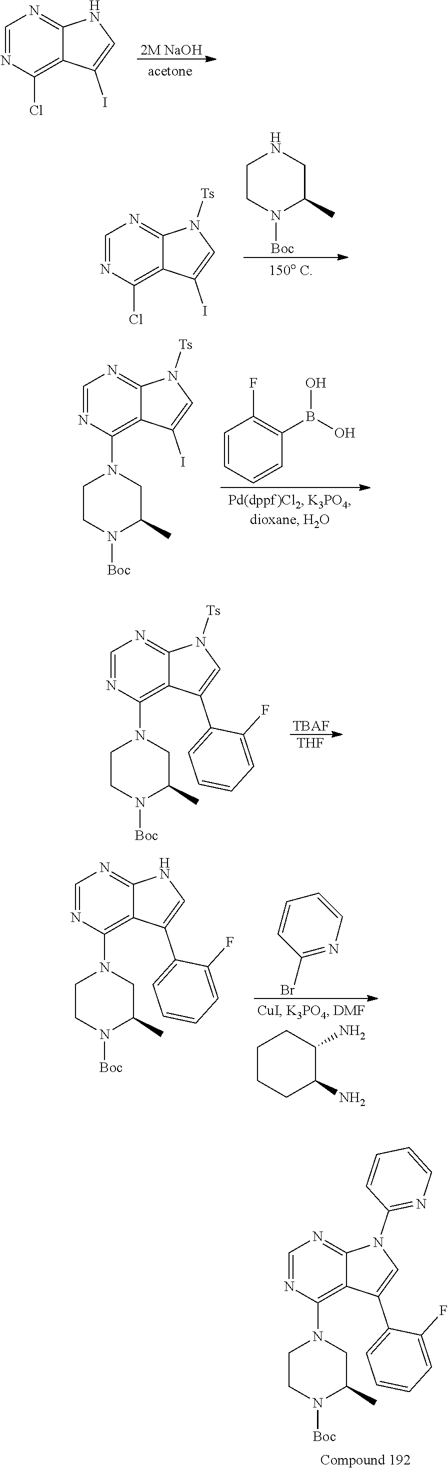

### Step 1. 4-Chloro-5-iodo-7-tosyl-7H-pyrrolo[2,3-d]pyrimidine

To a 0° C. solution of 4-chloro-5-iodo-7H-pyrrolo[2,3-d]pyrimidine (200 g, 0.71 mol) and 4-methylbenzene-1-sulfonyl chloride (180 g, 0.93 mol) in acetone (2 L) was added 2.0M NaOH (0.53 L) dropwise. After addition, the reaction was allowed to warm up to room temperature and stirred for another 3 hours. The precipitate was collected by filtration, and washed with water twice and dried under vacuum to give 4-chloro-5-iodo-7-tosyl-7H-pyrrolo[2,3-d]pyrimidine (300 g, 95%) as an off white solid. LC/MS ESI (m/z): 434 (M+H)⁺.

### Step 2. tert-Butyl (R)-4-(5-iodo-7-tosyl-7H-pyrrolo[2,3-d]pyrimidin-4-yl)-2-methylpiperazine-1-carboxylate

To a solution of 4-chloro-5-iodo-7-tosyl-7H-pyrrolo[2,3-d]pyrimidine (50 g, 120 mmol) in EtOH (500 mL) was added tert-butyl (R)-2-methylpiperazine-1-carboxylate (28 g, 140 mmol). The resulting mixture was stirred at 90° C. under N₂ atmosphere for 16 hours. After cooling to room temperature, the solvent was removed, and the residue was quenched with H₂O and EtOAc and the organic layer was separated. The aqueous phase was extracted with EtOAc twice, the combined organic layers were dried over Na₂SO₄, filtered and concentrated. The residue was triturated with petroleum ether/EtOAc (10:1) and filtered to afford tert-butyl (R)-4-(5-iodo-7-tosyl-7H-pyrrolo[2,3-d]pyrimidin-4-yl)-2-methylpiperazine-1-carboxylate (48 g, 70%) as a light yellow solid. LC/MS ESI (m/z): 598 (M+H)⁺.

### Step 3. tert-Butyl (R)-4-(5-(2-fluorophenyl)-7-tosyl-7H-pyrrolo[2,3-d]pyrimidin-4-yl)-2-methylpiperazine-1-carboxylate

To a solution of tert-butyl (R)-4-(5-iodo-7-tosyl-7H-pyrrolo[2,3-d]pyrimidin-4-yl)-2-methylpiperazine-1-carboxylate (300 mg, 0.50 mmol) in dioxane (10 mL) and water (1 mL) were added (2-fluorophenyl)boronic acid (77 mg, 0.55 mmol), K₃PO₄ (210 mg, 1.0 mmol), and Pd(dppf)Cl₂ (37 mg, 0.050 mmol). The resulting mixture was heated to 90° C. overnight. After being cooled down to room temperature, the solvent was removed and the residue was purified by flash column chromatography (silica gel, 0-60% EtOAc in petroleum ether) to afford tert-butyl (R)-4-(5-(2-fluorophenyl)-7-tosyl-7H-pyrrolo[2,3-d]pyrimidin-4-yl)-2-methylpiperazine-1-carboxylate (280 mg, 97%) as a white solid. LC/MS ESI (m/z): 566 (M+H)⁺.

### Step 4. tert-Butyl (R)-4-(5-(2-fluorophenyl)-7H-pyrrolo[2,3-d]pyrimidin-4-yl)-2-methylpiperazine-1-carboxylate

To a solution of tert-butyl (R)-4-(5-(2-fluorophenyl)-7-tosyl-7H-pyrrolo[2,3-d]pyrimidin-4-yl)-2-methylpiperazine-1-carboxylate (280 mg, 0.49 mmol) in THE (6 mL) were added TBAF (3.0 mL, 1.0M in THF). The reaction mixture was stirred at room temperature overnight. The solvent was removed, and the residue was purified by flash column chromatography (silica gel, 0˜64% EtOAc in petroleum ether) to afford tert-butyl (R)-4-(5-(2-fluorophenyl)-7H-pyrrolo[2,3-d]pyrimidin-4-yl)-2-methylpiperazine-1-carboxylate (170 mg, 87%) as a colorless oil. LC/MS ESI (m/z): 412 (M+H)⁺.

### Step 5. tert-Butyl (R)-4-(5-(2-fluorophenyl)-7-(pyridin-2-yl)-7H-pyrrolo[2,3-d]pyrimidin-4-yl)-2-methylpiperazine-1-carboxylate

To a solution of tert-butyl (R)-4-(5-(2-fluorophenyl)-7H-pyrrolo[2,3-d]pyrimidin-4-yl)-2-methylpiperazine-1-carboxylate (170 mg, 0.42 mmol) and 2-bromopyridine (79 mg, 0.50 mmol) in DMF (10 mL) were added trans-1,2-diaminocyclohexane (97 mg, 0.85 mmol), CuI (190 mg, 0.42 mmol) and K₃PO₄ (180 mg, 0.84 mmol). The resulting mixture was stirred at 120° C. for 18 hrs. After being cooled down to room temperature, the reaction partitioned between EtOAc, and water and the organic layer was separated. The aqueous layer was extracted with EtOAc twice, the combined organic layers were washed with brine, dried over Na₂SO₄, filtered and concentrated. The residue was purified by flash column chromatography (silica gel, 0˜30%, ethyl acetate in petroleum ether) to give crude product which was further purified by prep-HPLC (Gilson, C18, MeCN in water) to afford tert-butyl (R)-4-(5-(2-fluorophenyl)-7-(pyridin-2-yl)-7H-pyrrolo[2,3-d]pyrimidin-4-yl)-2-methylpiperazine-1-carboxylate (110 mg, 53%) as a solid. LC/MS ESI (m/z): 489 (M+H)⁺. ¹H NMR (400 MHz, CDCl₃) δ 8.79 (d, J=8.3 Hz, 1H), 8.56 (s, 1H), 8.52-8.48 (m, 1H), 8.25 (s, 1H), 7.90 (ddd, J=8.4, 7.4, 1.9 Hz, 1H), 7.50 (td, J=7.6, 1.7 Hz, 1H), 7.37 (tdd, J=7.2, 5.1, 1.8 Hz, 1H), 7.26-7.17 (m, 3H), 4.24 (s, 1H), 3.83 (d, J=13.0 Hz, 1H), 3.67-3.61 (m, 1H), 3.43 (d, J=13.2 Hz, 1H), 3.01 (dd, J=13.0, 3.9 Hz, 1H), 2.70 (td, J=12.3, 3.2 Hz, 1H), 2.55 (td, J=13.0, 3.0 Hz, 1H), 1.43 (s, 9H), 1.13 (d, J=6.8 Hz, 3H).

### Example 7. Synthesis of tert-butyl (S)-4-(7-(3-fluorophenyl)-5-(pyridin-3-yl)-7H-pyrrolo[2,3-d]pyrimidin-4-yl)-3-methylpiperazine-1-carboxylate (Compound 226)

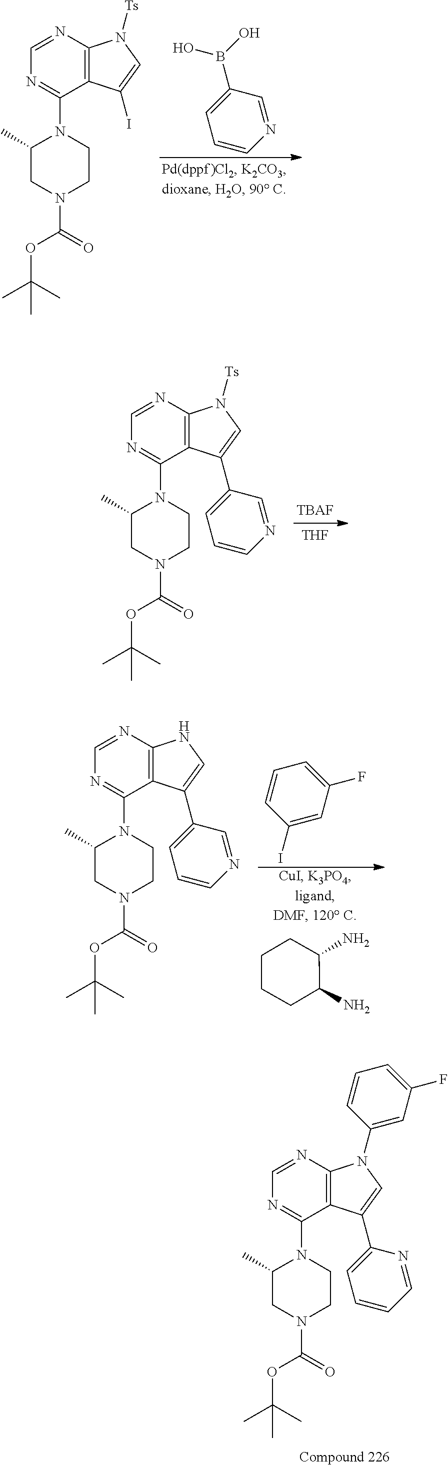

### Step 1. tert-Butyl (S)-3-methyl-4-(5-(pyridin-3-yl)-7-tosyl-7H-pyrrolo[2,3-d]pyrimidin-4-yl)piperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (700 mg, 1.2 mmol, prepared following the procedure compound 259, step 1) in dioxane (10 mL) and water (1 mL) were added pyridin-3-ylboronic acid (160 mg, 1.3 mmol), Pd(dppf)Cl₂ (86 mg, 0.12 mmol), and K₂CO₃ (320 mg, 2.3 mmol). The resulting mixture was heated to 90° C. overnight. After being cooled down to room temperature, the solvent was removed and the residue was purified by flash column chromatography (silica gel, 0˜50% EtOAc in petroleum ether) to afford tert-butyl (S)-3-methyl-4-(5-(pyridin-3-yl)-7-tosyl-7H-pyrrolo[2,3-d]pyrimidin-4-yl)piperazine-1-carboxylate (220 mg, 34%) as a light a yellow solid. LC/MS ESI (m/z): 549 (M+H)⁺.

### Step 2. tert-Butyl (S)-3-methyl-4-(5-(pyridin-3-yl)-7H-pyrrolo[2,3-d]pyrimidin-4-yl)piperazine-1-carboxylate

To a solution of tert-butyl (S)-3-methyl-4-(5-(pyridin-3-yl)-7-tosyl-7H-pyrrolo[2,3-d]pyrimidin-4-yl)piperazine-1-carboxylate (220 mg, 0.40 mmol) in THE (3 mL) was added TBAF (3.0 mL, 1.0M in THF). The resulting mixture was stirred at room temperature overnight. Solvent was removed and the residue was purified by flash column chromatography (silica gel, 0˜70% EtOAc in petroleum ether) to afford tert-butyl (S)-3-methyl-4-(5-(pyridin-3-yl)-7H-pyrrolo[2,3-d]pyrimidin-4-yl)piperazine-1-carboxylate (120 mg, 78%) as a light yellow solid. LC/MS ESI (m/z): 395 (M+H)⁺.

### Step 3. tert-Butyl (S)-4-(7-(3-fluorophenyl)-5-(pyridin-3-yl)-7H-pyrrolo[2,3-d]pyrimidin-4-yl)-3-methylpiperazine-1-carboxylate

To a solution of tert-butyl (S)-3-methyl-4-(5-(pyridin-3-yl)-7H-pyrrolo[2,3-d]pyrimidin-4-yl)piperazine-1-carboxylate (40 mg, 0.10 mmol) and 1-fluoro-3-iodobenzene (27 mg mL, 0.12 mmol) in DMF (6 mL) were added CuI (19 mg, 0.10 mmol), K₃PO₄ (43 mg, 0.20 mmol) and trans-1,2-diaminocyclohexane (23 mg, 0.20 mmol). The resulting mixture was stirred at 120° C. for 18 hrs. After being cooled down to room temperature, the reaction was partitioned between EtOAc and water, organic layer was separated, aqueous layer was extracted with EtOAc twice, the combined organic layers were washed with brine, filtered, and concentrated. The residue was purified by flash column chromatography (silica gel, 0˜50%, ethyl acetate in petroleum ether) to give crude product which was further purified by prep-HPLC to afford tert-butyl (S)-4-(7-(3-fluorophenyl)-5-(pyridin-3-yl)-7H-pyrrolo[2,3-d]pyrimidin-4-yl)-3-methylpiperazine-1-carboxylate (20 mg, 41%) as a light yellow solid. LC/MS ESI (m/z): 489 (M+H)⁺. ¹H NMR (400 MHz, CD₃OD) δ 8.81 (s, 1H), 8.57 (s, 1H), 8.46 (s, 1H), 8.10 (d, J=8.0 Hz, 1H), 7.87 (s, 1H), 7.72 (dt, J=10.2, 2.2 Hz, 1H), 7.66-7.63 (m, 1H), 7.61-7.55 (m, 2H), 7.18 (tdd, J=8.4, 2.4, 0.8 Hz, 1H), 4.07 (dt, J=9.8, 3.1 Hz, 1H), 3.80 (d, J=12.4 Hz, 1H), 3.50 (t, J=12.2 Hz, 2H), 3.20-3.13 (m, 1H), 2.95-2.78 (m, 2H), 1.42 (s, 9H), 0.98 (d, J=6.5 Hz, 3H).

The following compound was prepared by the procedure analogous to the synthesis of compound 226 from the corresponding aryl halide.

| Cmpd | | |
|---|---|---|
| No. | Chemical Name | LCMS and ¹H NMR |
| 225 | tert-butyl (S)-4-(7-(3- | LC/MS ESI (m/z): 505 (M + H)⁺. 1 H NMR (400 MHz, |
| | chlorophenyl)-5-(pyridin-3-yl)- | MeOD) δ 8.80 (d, J = 1.6 Hz, 1H), 8.56 (dd, J = 4.9, 1.5 Hz, |
| | 7H-pyrrolo[2,3-d]pyrimidin-4- | 1H), 8.46 (s, 1H), 8.12-8.08 (m, 1H), 7.95 (t, J = 2.0 Hz, |
| | yl)-3-methylpiperazine-1- | 1H), 7.86 (s, 1H), 7.75 (ddd, J = 8.1, 2.0, 0.9 Hz, 1H), 7.60- |
| | carboxylate | 7.53 (m, 2H), 7.45 (ddd, J = 8.1, 2.0, 0.9 Hz, 1H), 4.08 |
| | | (dt, J = 9.7, 3.2 Hz, 1H), 3.80 (d, J = 12.9 Hz, 1H), 3.54- |
| | | 3.46 (m, 2H), 3.20-3.13 (m, 1H), 2.98-2.76 (m, 2H), |
| | | 1.43 (s, 9H), 0.98 (d, J = 6.5 Hz, 3H). |

### Example 8. Synthesis of tert-butyl (S)-4-(5-cyclopropyl-7-(5-isocyanopyridin-3-yl)-7H-pyrrolo[2,3-d]pyrimidin-4-yl)-3-methylpiperazine-1-carboxylate (Compound 259)

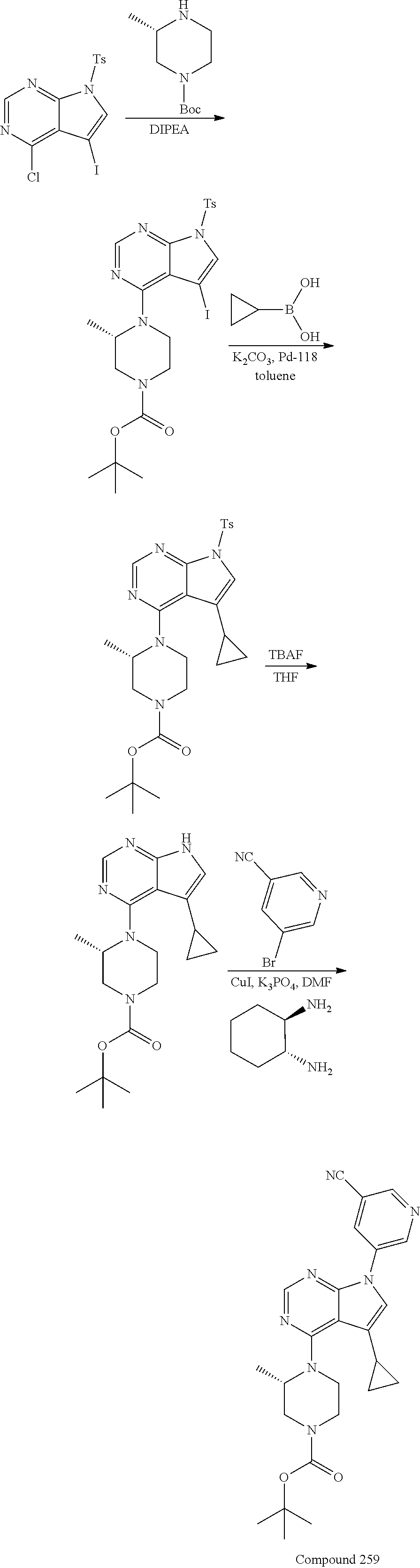

### Step 1. tert-Butyl (S)-4-(5-iodo-7-tosyl-7H-pyrrolo[2,3-d]pyrimidin-4-yl)-3-methylpiperazine-1-carboxylate

To the solution of 4-chloro-5-iodo-7-tosyl-7H-pyrrolo[2,3-d]pyrimidine (5.0 g, 12 mmol) in DIPEA (15 mL) was added tert-butyl (S)-3-methylpiperazine-1-carboxylate (5.8 g, 29 mmol). The resulting reaction mixture was stirred at 140° C. under N₂ for 2 h. After being cooled down to room temperature, the reaction mixture was concentrated under reduced pressure. The crude product was purified by flash column chromatography (silica gel, 0˜40%, ethyl acetate in petroleum ether) to afford tert-butyl (S)-4-(5-iodo-7-tosyl-7H-pyrrolo[2,3-d]pyrimidin-4-yl)-3-methylpiperazine-1-carboxylate (5.8 g, 54%) as a white solid. LC/MS ESI (m/z): 598 (M+H)⁺.

### Step 2. tert-Butyl (S)-4-(5-cyclopropyl-7-tosyl-7H-pyrrolo[2,3-d]pyrimidin-4-yl)-3-methylpiperazine-1-carboxylate

To the solution of tert-butyl (S)-4-(5-iodo-7-tosyl-7H-pyrrolo[2,3-d]pyrimidin-4-yl)-3-methylpiperazine-1-carboxylate (1.0 g, 1.7 mmol) in toluene (20 mL) were added Pd-(dtbp)Cl₂ (220 mg, 0.34 mmol), K₂CO₃ (3.0 g, 22 mmol) and cyclopropylboronic acid (220 mg, 2.5 mmol) respectively. The resulting reaction mixture was stirred at 80° C. for 4 h. After being cooled down to room temperature, the reaction mixture was filtered, and the filtrate was concentrated under reduced pressure. The residue was purified by flash column chromatography (silica gel, 0˜50%, ethyl acetate in petroleum ether) to afford tert-butyl (S)-4-(5-cyclopropyl-7-tosyl-7H-pyrrolo[2,3-d]pyrimidin-4-yl)-3-methylpiperazine-1-carboxylate (430 mg, 50%) as a white solid. LC/MS ESI (m/z): 512 (M+H)⁺.

### Step 3. tert-Butyl (S)-4-(5-cyclopropyl-7H-pyrrolo[2,3-d]pyrimidin-4-yl)-3-methylpiperazine-1-carboxylate

To the solution of tert-butyl (S)-4-(5-cyclopropyl-7-tosyl-7H-pyrrolo[2,3-d]pyrimidin-4-yl)-3-methylpiperazine-1-carboxylate (430 mg, 0.84 mmol) in THE (10 mL) was added TBAF (4.0 mL, 1.0M in THF). The resulting reaction mixture was stirred at r.t under N₂ overnight. The reaction mixture was quenched with ice water, then it was extracted with EtOAc twice, the combined organic layers were washed with water and brine, dried over Na₂SO₄, filtered, and concentrated under reduced pressure. The residue was purified by flash column chromatography (silica gel, 0˜80%, ethyl acetate in petroleum ether) to afford tert-butyl (S)-4-(5-cyclopropyl-7H-pyrrolo[2,3-d]pyrimidin-4-yl)-3-methylpiperazine-1-carboxylate (250 mg, 83%) as a white solid. LC/MS ESI (m/z): 358 (M+H)⁺.

### Step 4. tert-Butyl (S)-4-(5-cyclopropyl-7-(5-isocyanopyridin-3-yl)-7H-pyrrolo[2,3-d]pyrimidin-4-yl)-3-methylpiperazine-1-carboxylate

To the solution of tert-butyl (S)-4-(5-cyclopropyl-7H-pyrrolo[2,3-d]pyrimidin-4-yl)-3-methylpiperazine-1-carboxylate (70 mg, 0.20 mmol) in DMF (2 mL) were added CuI (37 mg, 0.20 mmol), K₃PO₄ (83 mg, 0.39 mmol), trans-cyclohexane-1,2-diamine (45 mL, 0.39 mmol) and 5-bromopyridine-3-carbonitrile (43 mg, 0.24 mmol) respectively. The resulting reaction mixture was stirred at 120° C. under N₂ overnight. After being cooled down to rt, the reaction mixture was concentrated under reduced pressure. The residue was purified by flash column chromatography (silica gel, 0˜40%, ethyl acetate in petroleum ether) and prep-HPLC (C-18, MeCN in H₂O) to afford tert-butyl (S)-4-(5-cyclopropyl-7-(5-isocyanopyridin-3-yl)-7H-pyrrolo[2,3-d]pyrimidin-4-yl)-3-methylpiperazine-1-carboxylate (28 mg, 31%) as white solid. LC/MS ESI (m/z): 460 (M+H)⁺. ¹H NMR (400 MHz, CD₃OD) δ 9.33 (d, J=2.5 Hz, 1H), 8.83 (d, J=1.7 Hz, 1H), 8.75-8.72 (m, 1H), 8.36 (s, 1H), 7.42 (d, J=0.8 Hz, 1H), 4.81-4.76 (m, 1H), 4.08 (d, J=14.4 Hz, 1H), 3.97-3.86 (m, 2H), 3.59-3.52 (m, 1H), 3.49-3.34 (m, 1H), 3.22-3.10 (m, 1H), 2.11-2.06 (m, 1H), 1.50 (s, 9H), 1.21 (d, J=6.6 Hz, 3H), 1.09-1.04 (m, 2H), 0.93-0.88 (m, 1H), 0.82-0.76 (m, 1H).

The following compounds were prepared by the procedures analogous to the synthesis of compound 259 from the corresponding aryl halides.

| Cmpd | | |
|---|---|---|
| No. | Chemical Name | LCMS and ¹H NMR |
| 260 | tert-butyl (S)-4-(5-cyclopropyl- | LC/MS ESI (m/z): 460 (M + H)⁺. ¹H NMR (400 MHz, |
| | 7-(4-isocyanopyridin-2-yl)-7H- | CD3OD) δ 9.20-9.16 (m, 1H), 8.65-8.62 (m, 1H), 8.44 |
| | pyrrolo[2,3-d]pyrimidin-4-yl)-3- | (s, 1H), 7.84 (d, J = 0.8 Hz, 1H), 7.55-7.49 (m, 1H), 4.76- |
| | methylpiperazine-1-carboxylate | 4.68 (m, 1H), 4.09-3.84 (m, 3H), 3.58-3.51 (m, 1H), |
| | | 3.48-3.34 (m, 1H), 3.23-3.08 (m, 1H), 2.11-2.04 (m, |
| | | 1H), 1.50 (s, 9H), 1.20 (d, J = 6.6 Hz, 3H), 1.09-1.04 (m, |
| | | 2H), 0.89-0.84 (m, 1H), 0.77-0.72 (m, 1H). |
| 264 | tert-butyl (S)-4-(5-cyclopropyl- | LC/MS ESI (m/z): 548 (M + H)⁺. ¹H NMR (400 MHz, |
| | 7-(3-methoxy-5- | CDCl₃) δ 8.44 (s, 1H), 7.28 (t, J = 2.0 Hz, 1H), 7.14-7.12 |
| | (trifluoromethoxy)phenyl)-7H- | (m, 1H), 6.91 (s, 1H), 6.73-6.72 (m, 1H), 4.74 (d, J = 1.6 |
| | pyrrolo[2,3-d]pyrimidin-4-yl)-3- | Hz, 1H), 4.20-3.95 (m, 2H), 3.87 (s, 3H), 3.63-3.04 (m, |
| | methylpiperazine-1-carboxylate | 4H), 2.07-2.02 (m, 1H), 1.50 (s, 9H), 1.24 (d, J = 6.4 Hz, |
| | | 3H), 1.04-1.01 (m, 2H), 0.83-0.78 (m, 1H), 0.74-0.69 |
| | | (m, 1H). |
| 198 | tert-butyl (S)-4-(5-cyclopropyl- | LC/MS ESI (m/z): 500 (M + H)⁺. ¹H NMR (400 MHz, |
| | 7-(3-(difluoromethoxy)phenyl)- | CDCl₃) δ 8.44 (s, 1H), 7.55 (ddd, J = 8.0, 2.0, 1.0 Hz, 1H), |
| | 7H-pyrrolo[2,3-d]pyrimidin-4- | 7.48 (dd, J = 9.3, 6.8 Hz, 2H), 7.09 (d, J = 8.2 Hz, 1H), |
| | yl)-3-methylpiperazine-1- | 6.92 (s, 1H), 6.59 (t, J = 73.6 Hz, 1H), 4.74 (s, 1H), 4.14- |
| | carboxylate | 3.82 (m, 3H), 3.55(t, J = 12.9 Hz, 1H), 3.38-3.10 (m, 2H), |
| | | 2.07-2.01 (m, 1H), 1.50 (s, 9H), 1.24 (d, J = 6.6 Hz, 3H), |
| | | 1.02 (dd, J = 8.2, 1.9 Hz, 2H), 0.82-0.70 (m, 2H). |
| 202 | tert-butyl (S)-4-(5-cyclopropyl- | LC/MS ESI (m/z): 464 (M + H)⁺. ¹H NMR (400 MHz, |
| | 7-(3-methoxyphenyl)-7H- | CDCl₃) δ 8.44 (s, 1H), 7.41-7.37 (m, 1H), 7.20 (dd, J = |
| | pyrrolo[2,3-d]pyrimidin-4-yl)-3- | 7.2, 1.1 Hz, 2H), 6.93 (s, 1H), 6.90-6.86 (m, 1H), 4.73 (s, |
| | methylpiperazine-1-carboxylate | 1H), 4.18-3.89 (m, 2H), 3.86 (s, 3H), 3.85-3.75 (m, 1H), |
| | | 3.55 (t, J = 13.7 Hz, 1H), 3.43-3.13 (m, 2H), 2.05 (t, J = |
| | | 7.5 Hz, 1H), 1.50 (s, 9H), 1.24 (d, J = 6.5 Hz, 3H), 1.01 |
| | | (dd, J = 8.2, 1.8 Hz, 2H), 0.81-0.68 (m, 2H) |
| 196 | tert-butyl (S)-4-(5-cyclopropyl- | LC/MS ESI (m/z): 518 (M + H)⁺. ¹H NMR (400 MHz, |
| | 7-(3-(trifluoromethoxy)phenyl)- | CDCl₃) δ 8.44 (s, 1H), 7.67 (ddd, J = 8.1, 2.0, 0.8 Hz, 1H), |
| | 7H-pyrrolo[2,3-d]pyrimidin-4- | 7.58 (s, 1H), 7.51 (t, J = 8.2 Hz, 1H), 7.21-7.17 (m, 1H), |
| | yl)-3-methylpiperazine-1- | 6.92 (d, J = 0.8 Hz, 1H), 4.74 (s, 1H), 4.14-3.80 (m, 3H), |
| | carboxylate | 3.55 (t, J = 13.1 Hz, 1H), 3.39-3.27 (m, 1H), 3.21-3.07 |
| | | (m, 1H), 2.09-2.02 (m, 1H), 1.50 (s, 9H), 1.24 (d, J = 6.6 |
| | | Hz, 3H), 1.03 (dd, J = 8.2, 1.8 Hz, 2H), 0.82-0.69 (m, |
| | | 2H). |
| 200 | tert-butyl (S)-4-(7-(3- | LC/MS ESI (m/z): 459 (M + H)⁺. ¹H NMR (400 MHz, |
| | cyanophenyl)-5-cyclopropyl-7H- | CDCl₃) δ 8.43 (s, 1H), 8.04 (d, J = 1.1 Hz, 1H), 8.01-7.97 |
| | pyrrolo[2,3-d]pyrimidin-4-yl)-3- | (m, 1H), 7.62-7.59 (m, 2H), 6.93 (d, J = 0.6 Hz, 1H), 4.75 |
| | methylpiperazine-1-carboxylate | (s, 1H), 4.15-3.84 (m, 3H), 3.53 (d, J = 21.9 Hz, 1H), 3.24 |
| | | (ddd, J = 46.7, 22.8, 10.1 Hz, 2H), 2.09-2.02 (m, 1H), 1.50 |
| | | (s, 9H), 1.25 (d, J = 6.5 Hz, 3H), 1.04 (dd, J = 8.2, 1.8 Hz, |
| | | 2H), 0.77 (dd, J = 29.9, 5.0 Hz, 2H |
| 203 | tert-butyl (S)-4-(5-cyclopropyl- | LC/MS ESI (m/z): 470 (M + H)⁺. ¹H NMR (400 MHz, |
| | 7-(3,5-difluorophenyl)-7H- | CDCl₃) δ 8.45 (s, 1H), 7.35 (dd, J = 8.2, 2.1 Hz, 2H), 6.90 |
| | pyrrolo[2,3-d]pyrimidin-4-yl)-3- | (s, 1H), 6.76 (tt, J = 8.8, 2.3 Hz, 1H), 4.73 (s, 1H), 4.17- |
| | methylpiperazine-1-carboxylate | 3.83 (m, 3H), 3.55 (t, J = 12.8 Hz, 1H), 3.23 (ddd, J = 41.3, |
| | | 26.3, 13.9 Hz, 2H), 2.04 (dd, J = 11.9, 5.9 Hz, 1H), 1.50 (s, |
| | | 9H), 1.24 (d, J = 6.6 Hz, 3H), 1.03 (dd, J = 8.2, 1.8 Hz, |
| | | 2H), 0.82-0.70 (m, 2H). |
| 219 | tert-butyl (S)-4-(5-cyclopropyl- | LC/MS ESI (m/z): 470 (M + H)⁺. ¹H NMR (400 MHz, |
| | 7-(3,4-difluorophenyl)-7H- | CDCl₃) δ 8.43 (s, 1H), 7.59 (ddd, J = 11.1, 7.0, 2.5 Hz, |
| | pyrrolo[2,3-d]pyrimidin-4-yl)-3- | 1H), 7.41-7.35 (m, 1H), 7.30-7.24 (m, 1H), 6.87 (s, 1H), |
| | methylpiperazine-1-carboxylate | 4.74 (s, 1H), 3.99 (ddd, J = 47.1, 32.0, 13.0 Hz, 3H), 3.55 |
| | | (dd, J = 14.0, 11.4 Hz, 1H), 3.35 (dd, J = 24.2, 10.8 Hz, |
| | | 1H), 3.21-3.04 (m, 1H), 2.06-1.99 (m, 1H), 1.50 (s, 9H), |
| | | 1.24 (d, J = 6.6 Hz, 3H), 1.02 (dd, J = 8.2, 1.9 Hz, 2H), |
| | | 0.81-0.68 (m, 2H). |
| 217 | tert-butyl (S)-4-(5-cyclopropyl- | LC/MS ESI (m/z): 470 (M + H)⁺. ¹H NMR (400 MHz, |
| | 7-(2,3-difluorophenyl)-7H- | CDCl₃) δ 8.42 (s, 1H), 7.39 (ddd, J = 6.0, 4.4, 2.2 Hz, 1H), |
| | pyrrolo[2,3-d]pyrimidin-4-yl)-3- | 7.24-7.20 (m, 2H), 6.85 (d, J = 1.3 Hz, 1H), 4.78 (s, 1H), |
| | methylpiperazine-1-carboxylate | 4.00 (t, J = 54.8 Hz, 3H), 3.56 (t, J = 11.1 Hz, 1H), 3.23 (d, |
| | | J = 79.8 Hz, 2H), 2.03 (d, J = 5.2 Hz, 1H), 1.50 (s, 9H), 1.26 |
| | | (d, J = 6.6 Hz, 3H), 1.01 (dd, J = 8.2, 1.8 Hz, 2H), 0.73 |
| | | (ddd, J = 15.2, 9.3, 3.7 Hz, 2H). |
| 218 | tert-butyl (S)-4-(5-cyclopropyl- | LC/MS ESI (m/z): 470 (M + H)⁺. ¹H NMR (400 MHz, |
| | 7-(2,5-difluorophenyl)-7H- | CDCl₃) δ 8.42 (s, 1H), 7.40 (ddd, J = 8.8, 5.9, 3.1 Hz, 1H), |
| | pyrrolo[2,3-d]pyrimidin-4-yl)-3- | 7.25-7.19 (m, 1H), 7.09-7.03 (m, 1H), 6.87-6.85 (m, |
| | methylpiperazine-1-carboxylate | 1H), 4.77 (s, 1H), 4.15-3.80 (m, 3H), 3.56 (t, J = 9.4 Hz, |
| | | 1H), 3.38-3.08 (m, 2H), 2.06-2.00 (m, 1H), 1.50 (s, 9H), |
| | | 1.25 (d, J = 6.5 Hz, 3H), 1.01 (dd, J = 8.2, 1.8 Hz, 2H), |
| | | 0.80-0.69 (m, 2H) |
| 290 | tert-butyl (S)-4-(7-(3-cyano-4- | LC/MS ESI (m/z): 477.6 (M + H)⁺. ¹H NMR (400 MHz, |
| | fluorophenyl)-5-cyclopropyl- | CDCl₃) δ 8.42 (s, 1H), 8.00-7.94 (m, 2H), 7.38-7.32 (m, |
| | 7H-pyrrolo[2,3-d]pyrimidin-4- | 1H), 6.88 (d, J = 0.7 Hz, 1H), 4.85-4.66 (m, 1H), 4.22- |
| | yl)-3-methylpiperazine-1- | 3.79 (m, 3H), 3.64-3.46 (m, 1H), 3.39-3.05 (m, 2H), |
| | carboxylate | 2.10-1.93 (m, 1H), 1.50 (s, 9H), 1.25 (d, J = 6.5 Hz, 3H), |
| | | 1.07-1.01 (m, 2H), 0.84-0.69 (m, 2H). |
| 288 | tert-butyl (S)-4-(5-cyclopropyl- | LC/MS ESI (m/z): 482.6 (M + H)⁺. ¹H NMR (400 MHz, |
| | 7-(3-fluoro-5-methoxyphenyl)- | CDCl₃) δ 8.44 (s, 1H), 7.09-7.01 (m, 2H), 6.90 (d, J = 0.7 |
| | 7H-pyrrolo[2,3-d]pyrimidin-4- | Hz, 1H), 6.62-6.55 (m, 1H), 4.78-4.66 (m, 1H), 4.17- |
| | yl)-3-methylpiperazine-1- | 3.79 (m, 6H), 3.60-3.48 (m, 1H), 3.41-3.26 (m, 1H), |
| | carboxylate | 3.24-3.04 (m, 1H), 2.09-1.97 (m, 1H), 1.50 (s, 9H), 1.23 |
| | | (d, J = 6.5 Hz, 3H), 1.04-0.97 (m, 2H), 0.83-0.75 (m, |
| | | 1H), 0.73-0.64 (m, 1H). |
| 289 | tert-butyl (S)-4-(5-cyclopropyl- | LC/MS ESI (m/z): 494.6 (M + H)⁺. ¹H NMR (400 MHz, |
| | 7-(3,5-dimethoxyphenyl)-7H- | CDCl₃) δ 8.44 (s, 1H), 6.91 (s, 1H), 6.81 (d, J = 2.2 Hz, |
| | pyrrolo[2,3-d]pyrimidin-4-yl)-3- | 2H), 6.45-6.42 (m, 1H), 4.79-4.66 (m, 1H), 4.15-3.81 |
| | methylpiperazine-1-carboxylate | (m, 9H), 3.62-3.48 (m, 1H), 3.42-3.27 (m, 1H), 3.23- |
| | | 3.04 (m, 1H), 2.08-2.00 (m, 1H), 1.50 (s, 9H), 1.23 (d, J = |
| | | 6.5 Hz, 3H), 1.04-0.98 (m, 2H), 0.83-0.75 (m, 1H), 0.73- |
| | | 0.66 (m, 1H). |
| 292 | tert-butyl (S)-4-(5-cyclopropyl- | LC/MS ESI (m/z): 484.5 (M + H)⁺. ¹H NMR (400 MHz, |
| | 7-(3,5-dicyanophenyl)-7H- | CDCl₃) δ 8.48-8.38 (m, 3H), 7.81 (t, J = 1.3 Hz, 1H), 6.94 |
| | pyrrolo[2,3-d]pyrimidin-4-yl)-3- | (s, 1H), 4.82-4.70 (m, 1H), 4.18-3.79 (m, 3H), 3.64- |
| | methylpiperazine-1-carboxylate | 3.47 (m, 1H), 3.39-3.01 (m, 2H), 2.06-1.98 (m, 1H), |
| | | 1.50 (s, 9H), 1.26 (d, J = 6.5 Hz, 3H), 1.10-1.04 (m, 2H), |
| | | 0.84-0.72 (m, 2H). |
| 291 | tert-butyl (S)-4-(7-(3-cyano-5- | LC/MS ESI (m/z): 477.6 (M + H)⁺. ¹H NMR (400 MHz, |
| | fluorophenyl)-5-cyclopropyl- | CDCl₃) δ 8.44 (s, 1H), 7.90 (t, J = 5.1 Hz, 2H), 7.32-7.27 |
| | 7H-pyrrolo[2,3-d]pyrimidin-4- | (m, 1H), 6.92 (s, 1H), 4.86-4.60 (m, 1H), 4.24-3.74 (m, |
| | yl)-3-methylpiperazine-1- | 3H), 3.65-3.45 (m, 1H), 3.39-2.96 (m, 2H), 2.09-1.96 |
| | carboxylate | (m, 1H), 1.50 (s, 9H), 1.25 (d, J = 6.5 Hz, 3H), 1.09-1.02 |
| | | (m, 2H), 0.85-0.70 (m, 2H). |
| 301 | tert-butyl (S)-4-(5-cyclopropyl- | LC/MS ESI (m/z): 500.6 (M + H)⁺. ¹H NMR (400 MHz, |
| | 7-(3,4-difluoro-5- | CDCl₃) δ 8.43 (s, 1H), 7.16 (d, J = 6.4 Hz, 1H), 7.09-7.02 |
| | methoxyphenyl)-7H-pyrrolo[2,3- | (m, 1H), 6.87 (s, 1H), 4.85-4.70 (m, 1H), 4.14-3.90 (m, |
| | d]pyrimidin-4-yl)-3- | 6H), 3.64-3.50 (m, 1H), 3.37-3.07 (m, 2H), 2.06-1.98 |
| | methylpiperazine-1-carboxylate | (m, 1H), 1.50 (s, 9H), 1.25 (d, J = 5.9 Hz, 3H), 1.05-1.00 |
| | | (m, 2H), 0.82-0.69 (m, 2H). |
| 317 | tert-butyl (S)-4-(5-cyclopropyl- | LC/MS ESI (m/z): 482.6 (M + H)⁺. ¹H NMR (400 MHz, |
| | 7-(4-fluoro-3-methoxyphenyl)- | CDCl₃) δ 8.43 (s, 1H), 7.36-7.27 (m, 1H), 7.17 (dd, J = |
| | 7H-pyrrolo[2,3-d]pyrimidin-4- | 10.8, 8.7 Hz, 1H), 7.10-7.03 (m, 1H), 6.88 (s, 1H), 4.86- |
| | yl)-3-methylpiperazine-1- | 4.64 (m, 1H), 4.15-3.78 (m, 6H), 3.64-3.49 (m, 1H), |
| | carboxylate | 3.42-3.04 (m, 2H), 2.11-1.98 (m, 1H), 1.50 (s, 9H), 1.24 |
| | | (d, J = 6.4 Hz, 3H), 1.05-0.97 (m, 2H), 0.84-0.66 (m, |
| | | 2H). |
| 302 | tert-butyl (S)-4-(7-(3-cyano-5- | LC/MS ESI (m/z): 489.5 (M + H)⁺. ¹H NMR (400 MHz, |
| | methoxyphenyl)-5-cyclopropyl- | CDCl₃) δ 8.44 (s, 1H), 7.62-7.55 (m, 2H), 7.09 (s, 1H), |
| | 7H-pyrrolo[2,3-d]pyrimidin-4- | 6.92 (s, 1H), 4.85-4.66 (m, 1H), 4.18-3.84 (m, 6H), 3.65- |
| | yl)-3-methylpiperazine-1- | 3.47 (m, 1H), 3.44-3.03 (m, 2H), 2.07-1.98 (m, 1H), |
| | carboxylate | 1.50 (s, 9H), 1.25 (d, J = 6.0 Hz, 3H), 1.07-1.01 (m, 2H), |
| | | 0.84-0.69 (m, 2H). |
| 303 | tert-butyl (S)-4-(7-(3-cyano-5- | LC/MS ESI (m/z): 543.6 (M + H)⁺. ¹H NMR (400 MHz, |
| | (trifluoromethoxy)phenyl)-5- | CDCl₃) δ 8.45 (s, 1H), 8.09 (s, 1H), 7.98 (s, 1H), 7.42 (s, |
| | cyclopropyl-7H-pyrrolo[2,3- | 1H), 6.92 (s, 1H), 4.85-4.65 (m, 1H), 4.20-3.80 (m, 3H), |
| | d]pyrimidin-4-yl)-3- | 3.65-3.47 (m, 1H), 3.41-2.98 (m, 2H), 2.07-1.98 (m, |
| | methylpiperazine-1-carboxylate | 1H), 1.50 (s, 9H), 1.25 (d, J = 6.4 Hz, 3H), 1.10-1.01 (m, |
| | | 2H), 0.84-0.70 (m, 2H). |

### Example 9. Synthesis of 1,1,1-trifluoro-2-methylpropan-2-yl (S)-4-(7-(3-cyanophenyl)-5-cyclopropyl-7H-pyrrolo[2,3-d]pyrimidin-4-yl)-3-methylpiperazine-1-carboxylate (Compound 266)

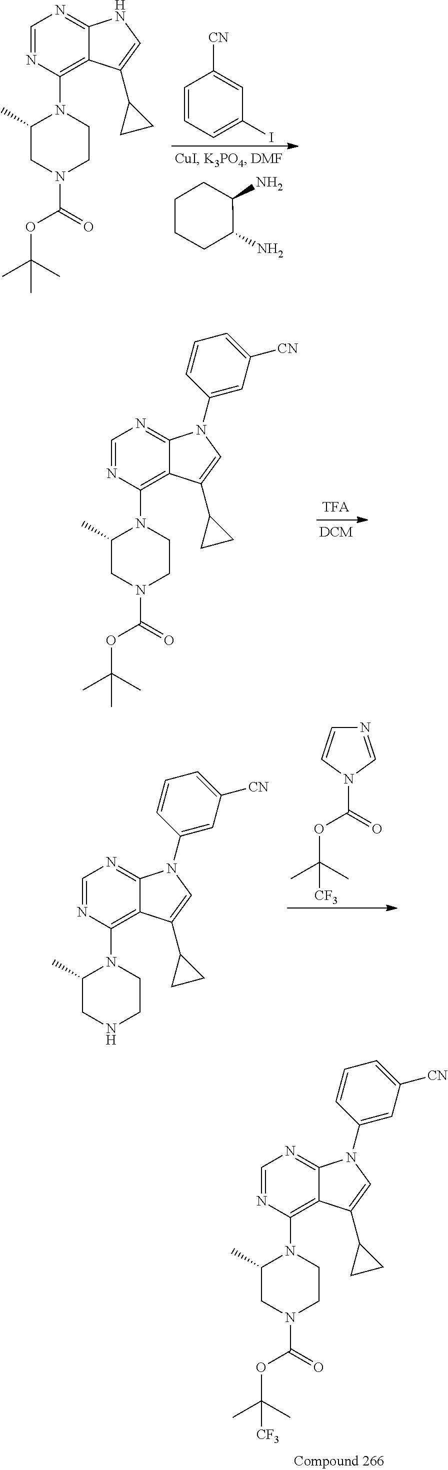

### Step 1. tert-Butyl (S)-4-(5-cyclopropyl-7-(5-isocyanopyridin-3-yl)-7H-pyrrolo[2,3-d]pyrimidin-4-yl)-3-methylpiperazine-1-carboxylate

To the solution of tert-butyl (S)-4-(5-cyclopropyl-7H-pyrrolo[2,3-d]pyrimidin-4-yl)-3-methylpiperazine-1-carboxylate (110 mg, 0.31 mmol) in DMF (3 mL) were added CuI (29 mg, 0.15 mmol), K₃PO₄ (200 mg, 0.92 mmol), trans-cyclohexane-1,2-diamine (22 mg, 0.18 mmol) and 2-bromopyridine-4-carbonitrile (140 mg, 0.62 mmol) respectively. The resulting reaction mixture was stirred at 120° C. under N₂ overnight. After being cooled down to room temperature, the reaction mixture was partitioned between EtOAc and water, organic layer was separated, aqueous layer was extracted with EtOAc twice, the combined organic layers were washed with brine, dried over Na₂SO₄, filtered and concentrated. The residue was purified by flash column chromatography (silica gel, 0˜50%, ethyl acetate in petroleum ether) to afford tert-butyl (S)-4-(7-(3-cyanophenyl)-5-cyclopropyl-7H-pyrrolo[2,3-d]pyrimidin-4-yl)-3-methylpiperazine-1-carboxylate (140 mg, 98%) as white solid. LC/MS ESI (m/z): 459 (M+H)⁺.

### Step 2. (S)-3-(5-Cyclopropyl-4-(2-methylpiperazin-1-yl)-7H-pyrrolo[2,3-d]pyrimidin-7-yl)benzonitrile

To the solution of tert-butyl (S)-4-(7-(3-cyanophenyl)-5-cyclopropyl-7H-pyrrolo[2,3-d]pyrimidin-4-yl)-3-methylpiperazine-1-carboxylate (140 mg, 0.30 mmol) in DCM (1 mL) was added TFA (0.50 mL, 6.7 mmol). The resulting reaction mixture was stirred at room temp under N₂ overnight. Solvent was removed and the residue was diluted with DCM, washed with sat. NaHCO₃, organic layer was dried over Na₂SO₄, filtered, and concentrated under reduced pressure to obtain the crude product which was used in the next step without further purification. LC/MS ESI (m/z): 359 (M+H)⁺.

### Step 3. 1,1,1-Trifluoro-2-methylpropan-2-yl (S)-4-(7-(3-cyanophenyl)-5-cyclopropyl-7H-pyrrolo[2,3-d]pyrimidin-4-yl)-3-methylpiperazine-1-carboxylate

To the solution of (S)-3-(5-cyclopropyl-4-(2-methylpiperazin-1-yl)-7H-pyrrolo[2,3-d]pyrimidin-7-yl)benzonitrile (75 mg, 0.21 mmol) in DMF (3 mL) were added DIPEA (0.20 mL, 1.0 mmol) and 1,1,1-trifluoro-2-methylpropan-2-yl-1H-imidazole-1-carboxylate (120 mg, 0.52 mmol, prepared from 1,1,1-trifluoro-2-methylpropan-2-ol and CDI) respectively. The resulting reaction mixture was stirred at 80° C. under N₂ overnight. After being cooled down to room temperature, the reaction mixture was partitioned between EtOAc and water. the organic layer was separated, and the aqueous layer was extracted EtOAc twice. The combined organic layers were washed with brine, dried over Na₂SO₄, filtered and concentrated. The residue was purified by prep-HPLC to afford 1,1,1-trifluoro-2-methylpropan-2-yl (S)-4-(7-(3-cyanophenyl)-5-cyclopropyl-7H-pyrrolo[2,3-d]pyrimidin-4-yl)-3-methylpiperazine-1-carboxylate (37 mg, 34%) as a white solid. LC/MS ESI (m/z): 513 (M+H)⁺. ¹H NMR (400 MHz, CD₃OD) δ 8.33 (s, 1H), 8.22-8.20 (m, 1H), 8.07-8.04 (m, 1H), 7.72-7.68 (m, 2H), 7.32 (d, J=0.6 Hz, 1H), 4.80 (s, 1H), 4.14-3.92 (m, 2H), 3.89-3.82 (m, 1H), 3.61-3.54 (m, 1H), 3.50-3.36 (m, 1H), 2.12-2.06 (m, 1H), 2.03 (s, 1H), 1.72 (d, J=6.3 Hz, 6H), 1.22 (d, J=6.5 Hz, 3H), 1.07-1.03 (m, 2H), 0.91-0.75 (m, 2H).

The following compounds were prepared by the procedures analogous to the synthesis of compound 266 from the corresponding aryl halides.

| Cmpd | | |
|---|---|---|
| No. | Chemical Name | LCMS and ¹H NMR |
| 265 | 1,1,1-trifluoro-2-methylpropan- | LC/MS ESI (m/z): 572 (M + H)⁺. ¹H NMR (400 MHz, |
| | 2-yl (S)-4-(5-cyclopropyl-7-(3- | CDCl₃) δ 8.46 (s, 1H), 7.68-7.64 (m, 1H), 7.57 (s, 1H), |
| | (trifluoromethoxy)phenyl)-7H- | 7.52 (t, J = 8.2 Hz, 1H), 7.20 (d, J = 8.3 Hz, 1H), 6.95 (s, |
| | pyrrolo[2,3-d]pyrimidin-4-yl)-3- | 1H), 4.77 (s, 1H), 4.16-3.94 (m, 2H), 3.90-3.80 (m, 1H), |
| | methylpiperazine-1-carboxylate | 3.59 (s, 1H), 3.45-3.33 (m, 1H), 3.29-3.10 (m, 1H), 2.06- |
| | | 2.00 (m, 1H), 1.73 (d, J = 7.2 Hz, 6H), 1.27-1.24 (m, |
| | | 3H), 1.05-1.01 (m, 2H), 0.83-0.70 (m, 2H). |
| 263 | 1,1,1-trifluoro-2-methylpropan- | LC/MS ESI (m/z): 518 (M + H)⁺. ¹H NMR (400 MHz, |
| | 2-yl (S)-4-(5-cyclopropyl-7-(3- | CDCl₃) δ 8.46 (s, 1H), 7.42-7.37 (m, 1H), 7.22-7.17 (m, |
| | methoxyphenyl)-7H-pyrrolo[2,3- | 2H), 6.96 (s, 1H), 6.92-6.87 (m, 1H), 4.78 (s, 1H), 4.23- |
| | d]pyrimidin-4-yl)-3- | 3.89 (m, 3H), 3.86 (s, 3H), 3.60 (s, 1H), 3.48-3.34 (m, |
| | methylpiperazine-1-carboxylate | 1H), 3.30-3.11 (m, 1H), 2.06-2.00 (m, 1H), 1.73 (d, J = |
| | | 7.2 Hz, 6H), 1.25 (d, J = 6.2 Hz, 3H), 1.01 (d, J = 8.2 Hz, |
| | | 2H), 0.83-0.69 (m, 2H). |

### Example 10. Synthesis of tert-butyl (S)-4-(7-(3-chlorophenyl)-5-(3-cyanopyrazin-2-yl)-7H-pyrrolo[2,3-d]pyrimidin-4-yl)-3-methylpiperazine-1-carboxylate (Compound 274)

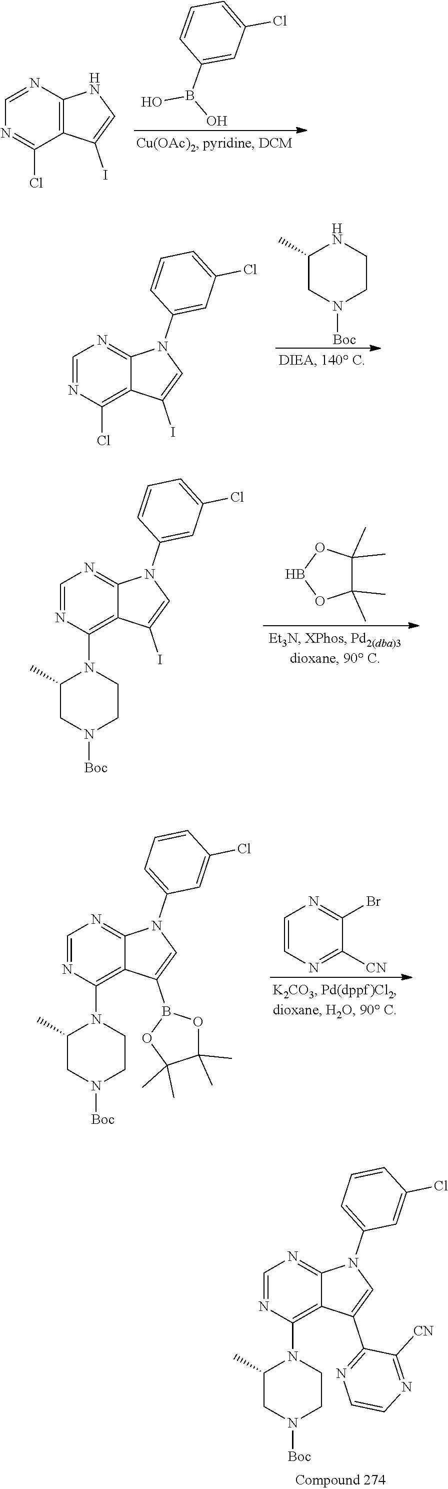

### Step 1. 4-Chloro-7-(3-chlorophenyl)-5-iodo-7H-pyrrolo[2,3-d]pyrimidine

To a solution of 4-chloro-5-iodo-7H-pyrrolo[2,3-d]pyrimidine (10 g, 36 mmol) in DCM (400 mL) were added (3-chlorophenyl)boronic acid (8.7 g, 72 mmol), 4 Å molecular sieves (5 g), Cu(OAc)₂ (16 g, 89 mmol) and pyridine (17 mL, 210 mmol). The resulting mixture was stirred at room temperature under O₂ atmosphere for 48 hours. The reaction was quenched with NH₄OH (30 mL) and ice water and filtered. The filtrate was extracted with DCM twice, the combined organic layers were dried over Na₂SO₄, filtered, and concentrated. The residue was purified by flash column chromatography (silica gel, 0˜30% EtOAc in petroleum ether) to afford 4-chloro-7-(3-chlorophenyl)-5-iodo-7H-pyrrolo[2,3-d]pyrimidine (7.5 g, 54%) as a white solid. LC/MS ESI (m/z): 390 (M+H)⁺.

### Step 2. tert-Butyl (S)-4-(7-(3-chlorophenyl)-5-iodo-7H-pyrrolo[2,3-d]pyrimidin-4-yl)-3-methylpiperazine-1-carboxylate

To the solution of 4-chloro-7-(3-chlorophenyl)-5-iodo-7H-pyrrolo[2,3-d]pyrimidine (2.5 g, 6.4 mmol) in DIEA (8 mL) was added tert-butyl (S)-3-methylpiperazine-1-carboxylate (3.2 g, 16 mmol). The resulting reaction mixture was stirred at 140° C. under N₂ for 2 h. After being cooled down to room temperature, the reaction mixture was concentrated under reduced pressure. The crude product was purified by flash column chromatography (silica gel, 0˜40%, ethyl acetate in petroleum ether) to afford tert-butyl (S)-4-(7-(3-chlorophenyl)-5-iodo-7H-pyrrolo[2,3-d]pyrimidin-4-yl)-3-methylpiperazine-1-carboxylate (3.0 g, 84%) as a white solid. LC/MS ESI (m/z): 554 (M+H)⁺.

### Step 3. tert-Butyl (S)-4-(7-(3-chlorophenyl)-5-iodo-7H-pyrrolo[2,3-d]pyrimidin-4-yl)-3-methylpiperazine-1-carboxylate

To the solution of tert-butyl (S)-4-(7-(3-chlorophenyl)-5-iodo-7H-pyrrolo[2,3-d]pyrimidin-4-yl)-3-methylpiperazine-1-carboxylate (2.5 g, 4.6 mmol) in dioxane (15 mL) were added X-Phos (220 mg, 0.46 mmol), Pd(dba)₃ (0.40 g, 0.46 mmol) and 4,4,5,5-tetramethyl-1,3,2-dioxaborolane (2.7 mL, 18 mmol) respectively. The resulting reaction mixture was stirred at 95° C. under N₂ overnight. After being cooled down to room temperature, the reaction mixture was quenched with ice water, extracted with DCM twice. The combined organic layers were dried over Na₂SO₄, filtered, and concentrated under reduced pressure to afford the crude product tert-butyl (S)-4-(7-(3-chlorophenyl)-5-(4,4,5,5-tetramethyl-1,3,2-dioxaborolan-2-yl)-7H-pyrrolo[2,3-d]pyrimidin-4-yl)-3-methylpiperazine-1-carboxylate (3.5 g) as a yellow oil that was used directly in the next step. LC/MS ESI (m/z): 554 (M+H)⁺.

### Step 4. tert-Butyl (S)-4-(7-(3-chlorophenyl)-5-(3-cyanopyrazin-2-yl)-7H-pyrrolo[2,3-d]pyrimidin-4-yl)-3-methylpiperazine-1-carboxylate

To the solution of tert-butyl (S)-4-(7-(3-chlorophenyl)-5-(4,4,5,5-tetramethyl-1,3,2-dioxaborolan-2-yl)-7H-pyrrolo[2,3-d]pyrimidin-4-yl)-3-methylpiperazine-1-carboxylate (70 mg, 0.13 mmol) in dioxane (2 mL) and H₂O (0.4 mL) were added K₂CO₃ (87 mg, 0.63 mmol), Pd(dppf)Cl₂ (10 mg, 0.01 mmol) and 3-bromopyrazine-2-carbonitrile (47 mg, 0.25 mmol) respectively. The resulting reaction mixture was stirred at 90° C. under N₂ overnight. After being cooled down to room temperature the solvent was removed. The residue was purified by flash column chromatography (silica gel, 0-20%, ethyl acetate in petroleum ether) and prep-HPLC (Gilson, C-18, MeCN in H₂O) to afford tert-butyl (S)-4-(7-(3-chlorophenyl)-5-(3-cyanopyrazin-2-yl)-7H-pyrrolo[2,3-d]pyrimidin-4-yl)-3-methylpiperazine-1-carboxylate (32 mg, 48%) as a white solid. LC/MS ESI (m/z): 531 (M+H)⁺. ¹H NMR (400 MHz, CDCl₃) δ 8.85 (d, J=2.3 Hz, 1H), 8.69 (d, J=2.3 Hz, 1H), 8.60 (s, 1H), 7.79 (s, 2H), 7.70-7.66 (m, 1H), 7.51 (t, J=8.0 Hz, 1H), 7.45-7.41 (m, 1H), 4.38-3.95 (m, 1H), 3.85-3.78 (m, 1H), 3.65-3.58 (m, 1H), 3.53-3.25 (m, 1H), 3.20-2.87 (m, 2H), 2.66 (s, 1H), 1.44 (s, 9H), 1.18-0.96 (m, 3H).

The following compounds were prepared by procedures analogous to the synthesis of compound 274 from the corresponding aryl halides.

| Cmpd | | |
|---|---|---|
| No. | Chemical Name | LCMS and ¹H NMR |
| 273 | tert-butyl (S)-4-(7-(3- | LC/MS ESI (m/z): 574 (M + H)⁺. ¹H NMR (400 MHz, |
| | chlorophenyl)-5-(3- | CDCl₃) δ 8.93-8.88 (m, 1H), 8.72 (d, J = 2.1 Hz, 1H), |
| | (trifluoromethyl)pyrazin-2-yl)- | 8.53 (s, 1H), 7.75 (s, 1H), 7.65-7.62 (m, 1H), 7.51-7.45 |
| | 7H-pyrrolo[2,3-d]pyrimidin-4- | (m, 2H), 7.42-7.38 (m, 1H), 4.34-4.14 (m, 1H), 3.74 (s, |
| | yl)-3-methylpiperazine-1- | 1H), 3.59-3.45 (m, 2H), 2.95 (s, 1H), 2.84-2.52 (m, 2H), |
| | carboxylate | 1.43 (s, 9H), 0.99 (s, 3H). |
| 275 | methyl (S)-3-(4-(4-(tert- | LC/MS ESI (m/z): 564 (M + H)⁺. ¹H NMR (400 MHz, |
| | butoxycarbonyl)-2- | CDCl₃) δ 8.82 (d, J = 2.3 Hz, 1H), 8.70 (d, J = 2.3 Hz, 1H), |
| | methylpiperazin-1-yl)-7-(3- | 8.58 (s, 1H), 7.82 (s, 1H), 7.72-7.69 (m, 1H), 7.66 (s, 1H), |
| | chlorophenyl)-7H-pyrrolo[2,3- | 7.49 (t, J = 8.1 Hz, 1H), 7.42-7.37 (m, 1H), 3.97-3.77 |
| | d]pyrimidin-5-yl)pyrazine-2- | (m, 2H), 3.75 (s, 3H), 3.52-3.35 (m, 2H), 3.23-3.07 (m, |
| | carboxylate | 1H), 2.98-2.55 (m, 2H), 1.42 (s, 9H), 0.98-0.84 (m, 3H). |

### Example 11. Synthesis of tert-butyl (R)-4-(5-(azetidin-1-yl)-7-(4-cyanopyridin-2-yl)-7H-pyrrolo[2,3-d]pyrimidin-4-yl)-2-methylpiperazine-1-carboxylate (Compound 388)

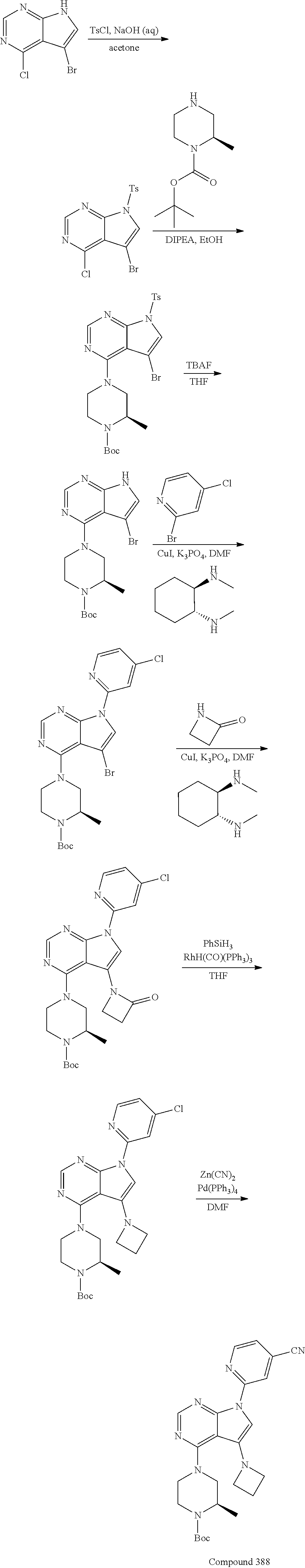

### Step 1. 5-Bromo-4-chloro-7-tosyl-7H-pyrrolo[2,3-d]pyrimidine

To 5-bromo-4-chloro-7H-pyrrolo[2,3-d]pyrimidine (5.0 g, 21 mmol) in acetone (70 mL) was added TsCl (4.1 g, 21 mmol). The mixture was cooled to 0° C. Then, 2M NaOH solution (13 mL) was added. After the addition, the mixture was stirred at rt for 3 h. The mixture was diluted with water and was filtered. The filter cake was washed with water and dried under vacuum to provide 5-bromo-4-chloro-7-tosyl-7H-pyrrolo[2,3-d]pyrimidine (7.4 g, 88%) as a white solid. LC/MS ESI (m/z): 386 (M+H)⁺.

### Step 2. tert-Butyl (R)-4-(5-bromo-7-tosyl-7H-pyrrolo[2,3-d]pyrimidin-4-yl)-2-methylpiperazine-1-carboxylate

A mixture of 5-bromo-4-chloro-7-tosyl-7H-pyrrolo[2,3-d]pyrimidine (3.0 g, 7.8 mmol), tert-butyl (R)-2-methylpiperazine-1-carboxylate (1.9 g, 9.3 mmol) and DIPEA (3.9 mL, 24 mmol) in EtOH (20 mL) was stirred at 100° C. overnight. The mixture was cooled to rt and filtered. The filter cake was washed with EtOH and dried under vacuum to provide tert-butyl (R)-4-(5-bromo-7-tosyl-7H-pyrrolo[2,3-d]pyrimidin-4-yl)-2-methylpiperazine-1-carboxylate (3.7 g, 86%) as a white solid. LC/MS ESI (m/z): 550 (M+H)⁺.

### Step 3. tert-Butyl (R)-4-(5-bromo-7H-pyrrolo[2,3-d]pyrimidin-4-yl)-2-methylpiperazine-1-carboxylate

tert-Butyl (R)-4-(5-bromo-7-tosyl-7H-pyrrolo[2,3-d]pyrimidin-4-yl)-2-methylpiperazine-1-carboxylate (3.7 g, 6.7 mmol) was treated with TBAF (16 mL, 1.0 M in THF) at rt for 2 h. The mixture was diluted with EtOAc, washed with brine, dried over Na₂SO₄ and concentrated. The residue was purified by flash column chromatography (silica gel, 0˜50% EtOAc in petroleum ether) to provide tert-butyl (R)-4-(5-bromo-7H-pyrrolo[2,3-d]pyrimidin-4-yl)-2-methylpiperazine-1-carboxylate (2.1 g, 77%) as a light yellow solid. LC/MS ESI (m/z): 396 (M+H)⁺.

### Step 4. tert-Butyl (R)-4-(5-bromo-7-(4-chloropyridin-2-yl)-7H-pyrrolo[2,3-d]pyrimidin-4-yl)-2-methylpiperazine-1-carboxylate

tert-Butyl (R)-4-(5-bromo-7H-pyrrolo[2,3-d]pyrimidin-4-yl)-2-methylpiperazine-1-carboxylate (2.1 g, 5.2 mmol), K₃PO₄ (2.2 g, 10 mmol), CuI (0.99 g, 5.2 mmol) and 2-bromo-4-chloropyridine (1.5 g, 7.8 mmol) were mixed in dry DMF (50 mL). trans-dimethylcyclohexane-1,2-diamine (0.74 g, 5.2 mmol) was then added, and the mixture was stirred at 90° C. under N₂ for 2.5 h. Then it was diluted with EtOAc, washed with LiCl (5% aq. solution) and brine, dried over Na₂SO₄ and concentrated. The residue was purified by flash column chromatography (silica gel, 0˜15% EtOAc in petroleum ether) to provide tert-butyl (R)-4-(5-bromo-7-(4-chloropyridin-2-yl)-7H-pyrrolo[2,3-d]pyrimidin-4-yl)-2-methylpiperazine-1-carboxylate (2.1 g, 79%). LC/MS ESI (m/z): 507 (M+H)⁺.

### Step 5. tert-Butyl (R)-4-(7-(4-chloropyridin-2-yl)-5-(2-oxoazetidin-1-yl)-7H-pyrrolo[2,3-d]pyrimidin-4-yl)-2-methylpiperazine-1-carboxylate

To tert-butyl (R)-4-(5-bromo-7-(4-chloropyridin-2-yl)-7H-pyrrolo[2,3-d]pyrimidin-4-yl)-2-methylpiperazine-1-carboxylate (300 mg, 0.59 mmol) and azetidin-2-one (130 mg, 1.8 mmol) were added K₃PO₄ (250 mg, 1.2 mmol), CuI (110 mg, 0.59 mmol), dry DMF (5 mL) and trans-dimethylcyclohexane-1,2-diamine (170 mg, 1.2 mmol). The mixture was stirred at 90° C. under N₂ overnight. It was then diluted with EtOAc, washed with 5% LiCl (aq) and brine, dried over Na₂SO₄ and concentrated. The residue was purified by flash column chromatography (0˜60% EtOAc in petroleum ether, then 0˜7% MeOH in DCM) to provide tert-butyl (R)-4-(7-(4-chloropyridin-2-yl)-5-(2-oxoazetidin-1-yl)-7H-pyrrolo[2,3-d]pyrimidin-4-yl)-2-methylpiperazine-1-carboxylate (170 mg, 58%) as a pale yellow solid. LC/MS ESI (m/z): 498 (M+H)⁺.

### Step 6. tert-Butyl (R)-4-(5-(azetidin-1-yl)-7-(4-chloropyridin-2-yl)-7H-pyrrolo[2,3-d]pyrimidin-4-yl)-2-methylpiperazine-1-carboxylate

To tert-butyl (R)-4-(7-(4-chloropyridin-2-yl)-5-(2-oxoazetidin-1-yl)-7H-pyrrolo[2,3-d]pyrimidin-4-yl)-2-methylpiperazine-1-carboxylate (65 mg, 0.13 mmol) in dry THE (4 mL) was added RhH(CO)(PPh₃)₃ (32 mg, 0.035 mmol). The mixture was purged with N₂. Then PhSiH₃ (70 mg, 0.65 mmol) was added. The mixture was stirred at 60° C. under N₂ for 2 h and was then concentrated and purified by prep-TLC (petroleum ether/EtOAc=5:1, v/v) to provide tert-butyl (2R)-4-[5-(azetidin-1-yl)-7-(4-chloropyridin-2-yl)-7H-pyrrolo[2,3-d]pyrimidin-4-yl]-2-methylpiperazine-1-carboxylate (50 mg, 79%) as a light yellow oil. LC/MS ESI (m/z): 484 (M+H)⁺.

### Step 7. tert-Butyl (R)-4-(5-(azetidin-1-yl)-7-(4-cyanopyridin-2-yl)-7H-pyrrolo[2,3-d]pyrimidin-4-yl)-2-methylpiperazine-1-carboxylate

To Zn(CN)₂ (75 mg, 0.64 mmol) and Pd(PPh₃)₄ (75 mg, 0.065 mmol) was added tert-butyl (2R)-4-[5-(azetidin-1-yl)-7-(4-chloropyridin-2-yl)-7H-pyrrolo[2,3-d]pyrimidin-4-yl]-2-methylpiperazine-1-carboxylate (62 mg, 0.13 mmol) in dry DMF (3 mL). The mixture was stirred at 120° C. under N₂ overnight. Then it was diluted with EtOAc, washed with 5% LiCl (aq) and brine, dried over Na₂SO₄ and concentrated. The residue was purified by prep-TLC (petroleum ether/EtOAc=3:1, v/v) to provide tert-butyl (R)-4-(5-(azetidin-1-yl)-7-(4-cyanopyridin-2-yl)-7H-pyrrolo[2,3-d]pyrimidin-4-yl)-2-methylpiperazine-1-carboxylate (16 mg, 26%) as a yellow solid. LC/MS ESI (m/z): 475 (M+H)⁺. ¹H NMR (400 MHz, CDCl₃) δ 9.35 (s, 1H), 8.57 (d, J=5.0 Hz, 1H), 8.41 (s, 1H), 7.46 (s, 1H), 7.29 (dd, J=5.0, 1.1 Hz, 1H), 4.76 (d, J=12.6 Hz, 1H), 4.46 (d, J=13.4 Hz, 1H), 4.40-4.29 (m, 1H), 3.95 (d, J=13.6 Hz, 1H), 3.83 (q, J=6.9 Hz, 2H), 3.64 (q, J=6.9 Hz, 2H), 3.46-3.33 (m, 2H), 3.02 (td, J=12.4, 3.4 Hz, 1H), 2.29 (p, J=7.1 Hz, 2H), 1.49 (s, 9H), 1.02 (d, J=6.8 Hz, 3H).

### Example 12. Synthesis of tert-butyl 7-(7-(4-cyanopyridin-2-yl)-5-(2-fluorophenyl)-7H-pyrrolo[2,3-d]pyrimidin-4-yl)-4,7-diazaspiro[2.5]octane-4-carboxylate (Compound 392)

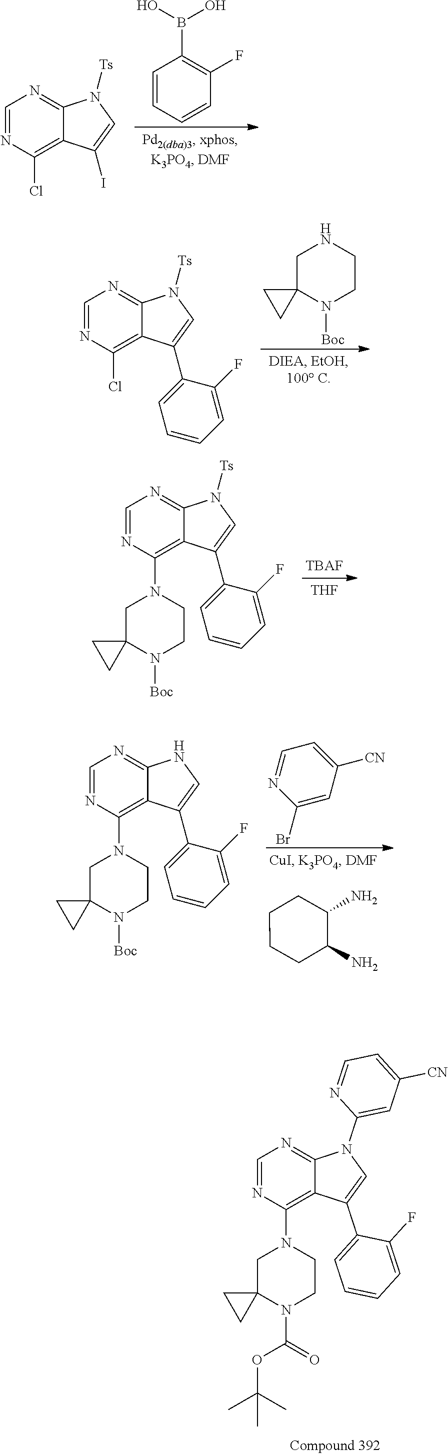

### Step 1. 4-Chloro-5-(2-fluorophenyl)-7-tosyl-7H-pyrrolo[2,3-d]pyrimidine

To a solution of 4-chloro-5-iodo-7-tosyl-7H-pyrrolo[2,3-d]pyrimidine (1.1 g, 2.5 mmol) in DMF (15 mL) and water (0.5 mL) were added (2-fluorophenyl)boronic acid (420 mg, 0.60 mmol), X-Phos (180 mg, 0.37 mmol), K₃PO₄ (1.6 g, 7.5 mmol) and Pd₂(dba)₃ (230 mg, 0.25 mmol), and the resulting mixture was heated to 60° C. overnight. After being cooled down to room temperature, the reaction mixture was filtered. The filtrate was partitioned between EtOAc and water and the organic layer was separated. The aqueous layer was extracted with EtOAc twice, the combined organic layers were washed with brine, dried over Na₂SO₄, filtered and concentrated. The residue was purified by flash column chromatography (silica gel, 0˜50% EtOAc in petroleum ether) to afford 4-chloro-5-(2-fluorophenyl)-7-tosyl-7H-pyrrolo[2,3-d]pyrimidine (600 mg, 60%). LC/MS ESI (m/z): 402 (M+H)⁺.

### Step 2. tert-Butyl 7-(5-(2-fluorophenyl)-7-tosyl-7H-pyrrolo[2,3-d]pyrimidin-4-yl)-4,7-diazaspiro[2.5]octane-4-carboxylate

To a solution of 4-chloro-5-(2-fluorophenyl)-7-tosyl-7H-pyrrolo[2,3-d]pyrimidine (550 mg, 2.2 mmol) in EtOH (6 mL) were added DIEA (0.66 mL, 4.0 mmol) and tert-butyl 4,7-diazaspiro[2.5]octane-4-carboxylate (440 mg, 2.1 mmol) respectively. The resulting reaction mixture was stirred at 100° C. under N₂ overnight. After being cooled down to room temperature, the reaction mixture was concentrated under reduced pressure. The residue was purified by flash column chromatography (silica gel, 0-30%, ethyl acetate in petroleum ether) to afford tert-butyl 7-(5-(2-fluorophenyl)-7-tosyl-7H-pyrrolo[2,3-d]pyrimidin-4-yl)-4,7-diazaspiro[2.5]octane-4-carboxylate (660 mg, 70%) as a white solid. LC/MS ESI (m/z): 578 (M+H)⁺.

### Step 3. tert-Butyl 7-(5-(2-fluorophenyl)-7H-pyrrolo[2,3-d]pyrimidin-4-yl)-4,7-diazaspiro[2.5]octane-4-carboxylate

To a solution of tert-butyl 7-(5-(2-fluorophenyl)-7-tosyl-7H-pyrrolo[2,3-d]pyrimidin-4-yl)-4,7-diazaspiro[2.5]octane-4-carboxylate (660 mg, 1.6 mmol) in THE (10 mL) was added TBAF (6.5 mL, 1.0 M in THF). The resulting reaction mixture was stirred at room temp under N₂ overnight. The reaction mixture was quenched with ice water and extracted with EtOAc twice. The combined organic layers were washed with brine, dried over Na₂SO₄, filtered, and concentrated under reduced pressure. The residue was purified by flash column chromatography (silica gel, 0˜60%, ethyl acetate in petroleum ether) to afford tert-butyl 7-(5-(2-fluorophenyl)-7H-pyrrolo[2,3-d]pyrimidin-4-yl)-4,7-diazaspiro[2.5]octane-4-carboxylate (410 mg, 62%) as a white solid. LC/MS ESI (m/z): 424 (M+H)⁺.

### Step 4. tert-Butyl 7-(7-(4-cyanopyridin-2-yl)-5-(2-fluorophenyl)-7H-pyrrolo[2,3-d]pyrimidin-4-yl)-4,7-diazaspiro[2.5]octane-4-carboxylate

To the solution of tert-butyl 7-(5-(2-fluorophenyl)-7H-pyrrolo[2,3-d]pyrimidin-4-yl)-4,7-diazaspiro[2.5]octane-4-carboxylate (400 mg, 0.95 mmol) in DMF (10 mL) were added CuI (90 mg, 0.47 mmol), K₃PO₄ (600 mg, 2.9 mmol), trans-cyclohexane-1,2-diamine (0.030 mL, 0.28 mmol) and 2-bromoisonicotinonitrile (350 mg, 1.9 mmol), respectively. The resulting reaction mixture was stirred at 80° C. under N₂ overnight. After being cooled down to room temperature, the reaction mixture was partitioned between EtOAc and water and the organic layer was separated. The aqueous layer was extracted with EtOAc twice, the combined organic layers were washed with brine, dried over Na₂SO₄, filtered and concentrated. The residue was purified by flash column chromatography (silica gel, 0˜30%, ethyl acetate in petroleum ether) to afford tert-butyl 7-(7-(4-cyanopyridin-2-yl)-5-(2-fluorophenyl)-7H-pyrrolo[2,3-d]pyrimidin-4-yl)-4,7-diazaspiro[2.5]octane-4-carboxylate (450 mg, 91%) as white solid. LC/MS ESI (m/z): 526 (M+H)⁺. ¹H NMR (400 MHz, CDCl₃) δ 9.40 (s, 1H), 8.63-8.60 (m, 1H), 8.54 (s, 1H), 8.28 (s, 1H), 7.50-7.46 (m, 1H), 7.41-7.35 (m, 2H), 7.28-7.18 (m, 2H), 3.28 (s, 2H), 3.21 (s, 2H), 3.12 (s, 2H), 1.42 (s, 9H), 0.93-0.88 (m, 2H), 0.74 (s, 2H).

### Example 13. Synthesis of tert-butyl (S)-4-(7-(3-chlorophenyl)-5-((R)-2-(hydroxymethyl)pyrrolidin-1-yl)-7H-pyrrolo[2,3-d]pyrimidin-4-yl)-3-methylpiperazine-1-carboxylate (Compound 312)

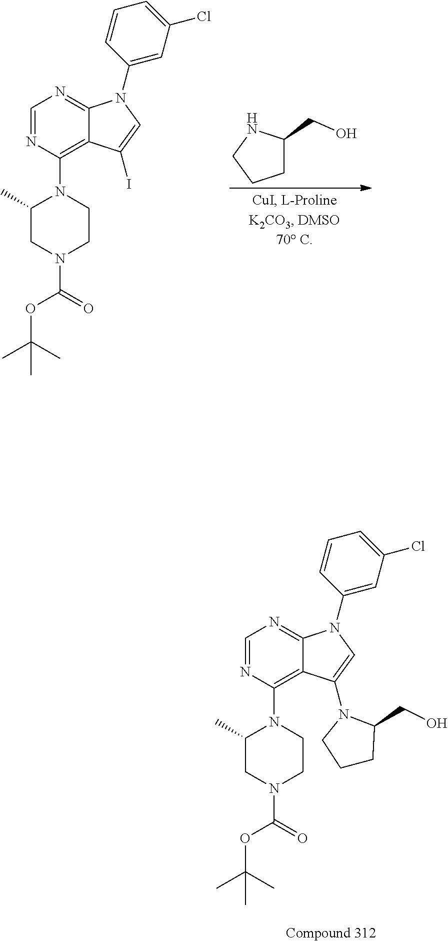 [SMILES: C[C@H]1CN(C(=O)OC(C)(C)C)CCN1c1ncnc2c1c(I)cn2-c1cccc(Cl)c1 ; OC[C@H]1CCCN1 ; C[C@H]1CN(C(=O)OC(C)(C)C)CCN1c1ncnc2c1c(N1CCC[C@@H]1CO)cn2-c1cccc(Cl)c1]

To a solution of tert-butyl (S)-4-(7-(3-chlorophenyl)-5-iodo-7H-pyrrolo[2,3-d]pyrimidin-4-yl)-3-methylpiperazine-1-carboxylate (460 mg, 0.83 mmol) in DMSO (5 mL) were added CuI (32 mg, 0.17 mmol), L-Proline (38 mg, 0.30 mmol), K₂CO₃ (340 mg, 2.5 mmol) and (R)-pyrrolidin-2-ylmethanol (0.33 mL, 3.3 mmol) respectively. The resulting reaction mixture was stirred at 70° C. under N₂ overnight. After being cooled down to room temperature, the reaction mixture was partitioned between EtOAc and water. The organic layer was separated and the aqueous was extracted with EtOAc twice. The combined organic layers were washed with brine, dried over Na₂SO₄, filtered, and concentrated under reduced pressure. The residue was purified by flash column chromatography (silica gel, 0-60%, ethyl acetate in petroleum ether) followed by prep-HPLC (Gilson, C-18, MeCN in water) to afford tert-butyl (S)-4-(7-(3-chlorophenyl)-5-((R)-2-(hydroxymethyl)pyrrolidin-1-yl)-7H-pyrrolo[2,3-d]pyrimidin-4-yl)-3-methylpiperazine-1-carboxylate (100 mg, 23%) as a solid. LC/MS ESI (m/z): 527 (M+H)⁺. ¹H NMR (400 MHz, CDCl₃) δ 8.41 (s, 1H), 7.74-7.68 (m, 1H), 7.67-7.62 (m, 1H), 7.43 (t, J=8.1 Hz, 1H), 7.33-7.28 (m, 1H), 6.90 (s, 1H), 5.19-5.01 (m, 1H), 4.23-3.92 (m, 3H), 3.91-3.82 (m, 1H), 3.79-3.69 (m, 2H), 3.65-3.61 (m, 1H), 3.55-3.43 (m, 2H), 3.41-3.29 (m, 1H), 3.04-2.89 (m, 1H), 2.82-2.74 (m, 1H), 2.19-2.12 (m, 1H), 2.00-1.88 (m, 3H), 1.49 (s, 9H), 1.18-1.04 (m, 3H).

### Example 14. Synthesis of tert-butyl 7-(7-(4-cyanopyridin-2-yl)-5-cyclopropyl-7H-pyrrolo[2,3-d]pyrimidin-4-yl)-4,7-diazaspiro[2.5]octane-4-carboxylate (Compound 396)

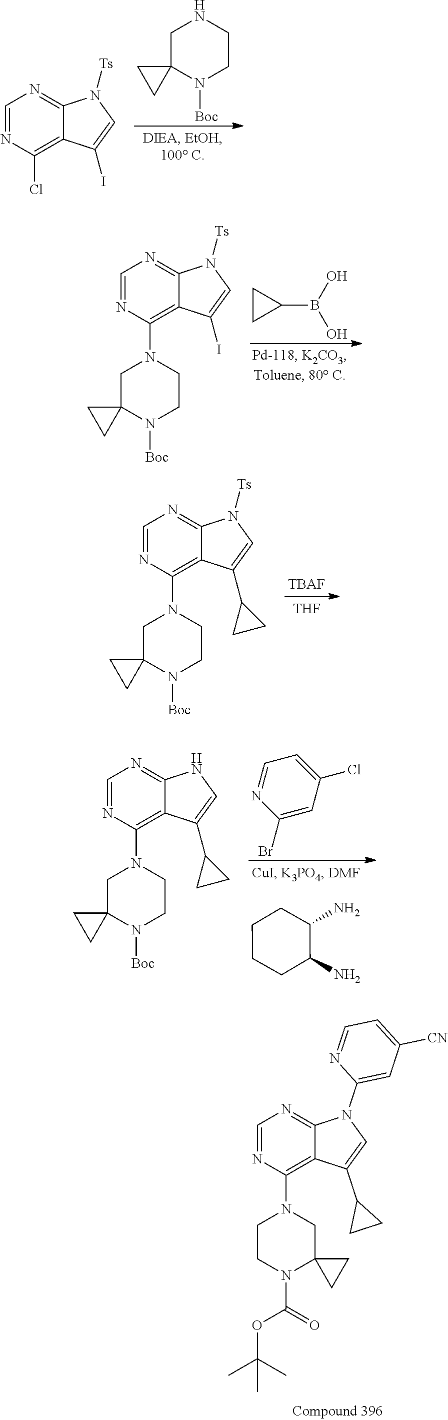

### Step 1. tert-Butyl 7-(5-iodo-7-tosyl-7H-pyrrolo[2,3-d]pyrimidin-4-yl)-4,7-diazaspiro[2.5]octane-4-carboxylate

To a solution of 4-chloro-5-iodo-7-tosyl-7H-pyrrolo[2,3-d]pyrimidine (750 mg, 2.7 mmol, prepared following the procedure of compound 192, step 1) in EtOH (5 mL) were added DIEA (0.90 mL, 5.5 mmol) and tert-butyl 4,7-diazaspiro[2.5]octane-4-carboxylate (550 mg, 2.6 mmol), respectively. The resulting reaction mixture was stirred at 100° C. under N₂ overnight. After being cooled down to room temperature, the reaction mixture was concentrated under reduced pressure. The residue was purified by flash column chromatography (silica gel, 0-30%, ethyl acetate in petroleum ether) to afford tert-butyl 7-(5-iodo-7-tosyl-7H-pyrrolo[2,3-d]pyrimidin-4-yl)-4,7-diazaspiro[2.5]octane-4-carboxylate (920 mg, 75%) as white solid. LC/MS ESI (m/z): 610 (M+H)⁺.

### Step 2. tert-Butyl 7-(5-cyclopropyl-7-tosyl-7H-pyrrolo[2,3-d]pyrimidin-4-yl)-4,7-diazaspiro[2.5]octane-4-carboxylate

To the solution of tert-butyl 7-(5-iodo-7-tosyl-7H-pyrrolo[2,3-d]pyrimidin-4-yl)-4,7-diazaspiro[2.5]octane-4-carboxylate (900 mg, 2.0 mmol) in toluene (12 mL) were added Pd-118 (97 mg, 0.15 mmol), K₂CO₃ (2.6 g, 19 mmol) and cyclopropylboronic acid (190 mg, 2.2 mmol) respectively. The resulting reaction mixture was stirred at 80° C. under N₂ overnight. After being cooled down to room temperature, the reaction mixture was concentrated under reduced pressure. The residue was purified by flash column chromatography (silica gel, 0˜40%, ethyl acetate in petroleum ether) to afford tert-butyl 7-(5-cyclopropyl-7-tosyl-7H-pyrrolo[2,3-d]pyrimidin-4-yl)-4,7-diazaspiro[2.5]octane-4-carboxylate (240 mg, 33%) as white solid. LC/MS ESI (m/z): 524 (M+H)⁺.

### Step 3. tert-Butyl (S)-4-(5-cyclopropyl-7H-pyrrolo[2,3-d]pyrimidin-4-yl)-3-methylpiperazine-1-carboxylate

To the solution of tert-butyl 7-(5-cyclopropyl-7-tosyl-7H-pyrrolo[2,3-d]pyrimidin-4-yl)-4,7-diazaspiro[2.5]octane-4-carboxylate (240 mg, 0.65 mmol) in THE (4 mL) was added TBAF (2.4 mL, 1.0 M in THF). The resulting reaction mixture was stirred at rt under N₂ overnight. The reaction mixture was quenched with ice water, and extracted with EtOAc twice, the combined organic layers were washed with brine, dried over Na₂SO₄, filtered, and concentrated under reduced pressure. The residue was purified by flash column chromatography (silica gel, 0˜50%, ethyl acetate in petroleum ether) to obtain tert-butyl 7-(5-cyclopropyl-7H-pyrrolo[2,3-d]pyrimidin-4-yl)-4,7-diazaspiro[2.5]octane-4-carboxylate (150 mg, 62%) as a white solid. LC/MS ESI (m/z): 370 (M+H)⁺.

### Step 4. tert-Butyl 7-(7-(4-cyanopyridin-2-yl)-5-cyclopropyl-7H-pyrrolo[2,3-d]pyrimidin-4-yl)-4,7-diazaspiro[2.5]octane-4-carboxylate

To a solution of tert-butyl 7-(5-cyclopropyl-7H-pyrrolo[2,3-d]pyrimidin-4-yl)-4,7-diazaspiro[2.5]octane-4-carboxylate (150 mg, 0.41 mmol) in DMF (5 mL) were added CuI (39 mg, 0.21 mmol), K₃PO₄ (260 mg, 1.2 mmol), trans-cyclohexane-1,2-diamine (0.020 mL, 0.12 mmol) and 2-bromoisonicotinonitrile (150 mg, 0.81 mmol) respectively. The resulting reaction mixture was stirred at 100° C. under N₂ overnight. After being cooled down to room temperature, the reaction mixture was partitioned between EtOAc and water, organic layer was separated, the aqueous layer was extracted with EtOAc twice, the combined organic layers were washed with brine, dried over Na₂SO₄, filtered and concentrated. The residue was purified by flash column chromatography (silica gel, 0˜30%, ethyl acetate in petroleum ether) and prep-HPLC to afford tert-butyl 7-(7-(4-cyanopyridin-2-yl)-5-cyclopropyl-7H-pyrrolo[2,3-d]pyrimidin-4-yl)-4,7-diazaspiro[2.5]octane-4-carboxylate (100 mg, 54%) as white solid. LC/MS ESI (m/z): 472 (M+H)⁺. ¹H NMR (400 MHz, CDCl₃) δ 9.32 (s, 1H), 8.59-8.56 (m, 1H), 8.47 (s, 1H), 7.77 (d, J=0.7 Hz, 1H), 7.35-7.32 (m, 1H), 3.87-3.81 (m, 2H), 3.77-3.73 (m, 2H), 3.61 (s, 2H), 2.02-1.95 (m, 1H), 1.50 (s, 9H), 1.06-0.98 (m, 4H), 0.83-0.78 (m, 4H).

### Example 15. Synthesis of tert-butyl (S)-4-(7-(4-cyano-6-methylpyridin-2-yl)-5-(2-fluorophenyl)-7H-pyrrolo[2,3-d]pyrimidin-4-yl)-3-methylpiperazine-1-carboxylate (Compound 404)

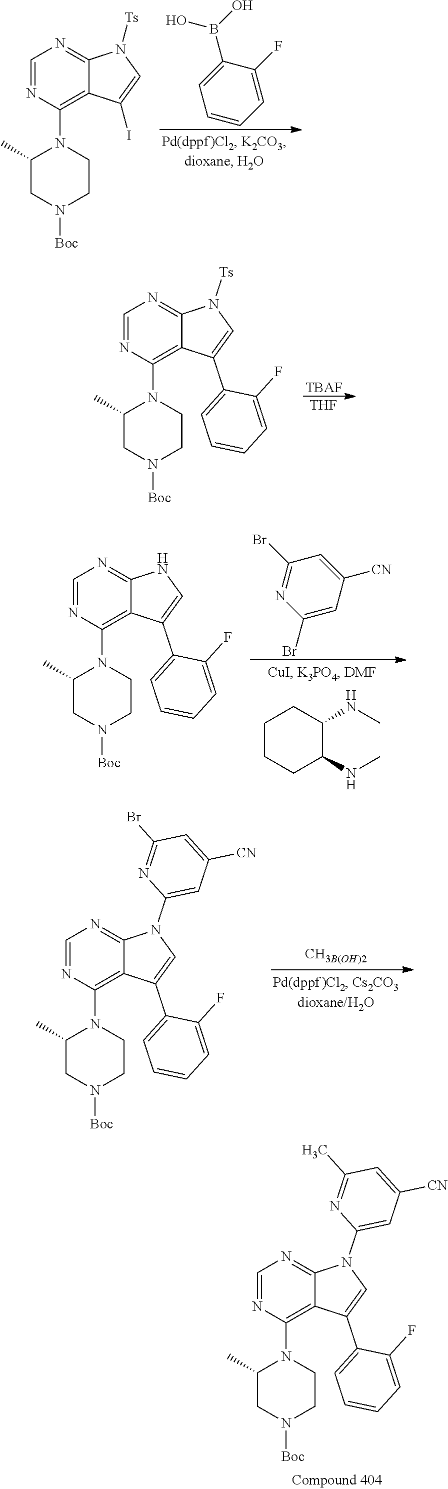

### Step 1. tert-Butyl (S)-4-(7-(6-bromo-4-cyanopyridin-2-yl)-5-(2-fluorophenyl)-7H-pyrrolo[2,3-d]pyrimidin-4-yl)-3-methylpiperazine-1-carboxylate

To the solution of tert-butyl (S)-4-(5-iodo-7-tosyl-7H-pyrrolo[2,3-d]pyrimidin-4-yl)-3-methylpiperazine-1-carboxylate (1.0 g, 1.7 mmol) in dioxane (20 mL) and water (0.5 mL) were added Pd(dppf)Cl₂ (140 mg, 0.17 mmol), K₂CO₃ (930 mg, 6.7 mmol) and (2-fluorophenyl)boronic acid (280 mg, 2.0 mmol). The resulting reaction mixture was stirred at 90° C. overnight. After being cooled down to room temperature, solvent was removed, the residue was purified by flash column chromatography (silica gel, 0-40%, ethyl acetate in petroleum ether) to obtain tert-butyl (S)-4-(5-(2-fluorophenyl)-7-tosyl-7H-pyrrolo[2,3-d]pyrimidin-4-yl)-3-methylpiperazine-1-carboxylate (810 mg, 85%) as a white solid. LC/MS ESI (m/z): 566 (M+H)⁺.

### Step 2. tert-Butyl (S)-4-(5-(2-fluorophenyl)-7H-pyrrolo[2,3-d]pyrimidin-4-yl)-3-methylpiperazine-1-carboxylate

To the solution of tert-butyl (S)-4-(5-(2-fluorophenyl)-7-tosyl-7H-pyrrolo[2,3-d]pyrimidin-4-yl)-3-methylpiperazine-1-carboxylate (570 mg, 1.0 mmol) in THE (4 mL) was added TBAF (4.0 mL, 1.0M in THF). The resulting reaction mixture was stirred at room temperature under N₂ overnight. The reaction mixture was quenched with ice water, then it was extracted with EtOAc twice, the combined organic layers were washed with water and brine, dried over Na₂SO₄, filtered, and concentrated under reduced pressure. The residue was purified by flash column chromatography (silica gel, 0˜60%, ethyl acetate in petroleum ether) to obtain tert-butyl (S)-4-(5-(2-fluorophenyl)-7H-pyrrolo[2,3-d]pyrimidin-4-yl)-3-methylpiperazine-1-carboxylate (330 mg, 80%) as a white solid. LC/MS ESI (m/z): 412 (M+H)⁺.

### Step 3. tert-Butyl (S)-4-(7-(6-bromo-4-cyanopyridin-2-yl)-5-(2-fluorophenyl)-7H-pyrrolo[2,3-d]pyrimidin-4-yl)-3-methylpiperazine-1-carboxylate

In a sealed tube, tert-butyl (S)-4-(5-(2-fluorophenyl)-7H-pyrrolo[2,3-d]pyrimidin-4-yl)-3-methylpiperazine-1-carboxylate (150 mg, 0.37 mmol), 2,6-dibromoisonicotinonitrile (190 mg, 0.73 mmol), K₃PO₄ (160 mg, 0.73 mmol) and CuI (69 mg, 0.36 mmol) were mixed in dry DMF. Then (±)-trans-1,2-cyclohexanediamine (100 mg, 0.73 mmol) was added. The mixture was stirred at 90° C. under N₂ for 3.5 h and was cooled to rt. Then it was diluted with EtOAc, washed with 5% LiCl (aq.) and brine, dried over Na₂SO₄ and concentrated. The residue was purified by flash column chromatography (silica gel, 0-11% EtOAc in petroleum ether) to provide tert-butyl (S)-4-(7-(6-bromo-4-cyanopyridin-2-yl)-5-(2-fluorophenyl)-7H-pyrrolo[2,3-d]pyrimidin-4-yl)-3-methylpiperazine-1-carboxylate (120 mg, 55%) as a yellow foam. LC/MS ESI (m/z): 592 (M+H)⁺.

### Step 4. tert-Butyl (S)-4-(7-(4-cyano-6-methylpyridin-2-yl)-5-(2-fluorophenyl)-7H-pyrrolo[2,3-d]pyrimidin-4-yl)-3-methylpiperazine-1-carboxylate

A mixture of tert-butyl (S)-4-(7-(6-bromo-4-cyanopyridin-2-yl)-5-(2-fluorophenyl)-7H-pyrrolo[2,3-d]pyrimidin-4-yl)-3-methylpiperazine-1-carboxylate (120 mg, 0.20 mmol), methylboronic acid (98 mg, 1.6 mmol), Cs₂CO₃ (200 mg, 0.61 mmol) and Pd(dppf)Cl₂ (20 mg, 0.027 mmol) in dioxane (3 mL) and H₂O (0.6 mL) was stirred at 100° C. under N₂ overnight. Then it was diluted with EtOAc and DCM and was filtered. The filtrate was purified by flash column chromatography (silica gel, 0˜20% EtOAc in petroleum ether followed by prep-HPLC to provide tert-butyl (S)-4-(7-(4-cyano-6-methylpyridin-2-yl)-5-(2-fluorophenyl)-7H-pyrrolo[2,3-d]pyrimidin-4-yl)-3-methylpiperazine-1-carboxylate (23 mg, 21%) as a light yellow oil. LC/MS ESI (m/z): 528 (M+H)⁺. ¹H NMR (400 MHz, CDCl₃) δ 9.16 (s, 1H), 8.56 (s, 1H), 8.29 (s, 1H), 7.46 (td, J=7.6, 1.5 Hz, 1H), 7.37 (tdd, J=7.2, 5.1, 1.8 Hz, 1H), 7.26-7.17 (m, 3H), 4.32-4.06 (m, 1H), 3.86-3.61 (m, 1H), 3.56-3.42 (m, 2H), 3.08 (td, J=12.3, 2.5 Hz, 1H), 2.87-2.59 (m, 5H), 1.43 (s, 9H), 1.01 (d, J=5.7 Hz, 3H).

The following compound was prepared by a synthetic procedure similar to that described for compound 404, except using tert-butyl (S)-4-(5-cyclopropyl-7H-pyrrolo[2,3-d]pyrimidin-4-yl)-3-methylpiperazine-1-carboxylate.

| Cmpd | | |
|---|---|---|
| No. | Chemical Name | LCMS and ¹H NMR |
| 403 | tert-butyl (S)-4-(7-(4-cyano-6- | LC/MS ESI (m/z): 474 (M + H)⁺. ¹H NMR (400 MHz, |
| | methylpyridin-2-yl)-5- | CDCl₃) δ 9.07 (s, 1H), 8.50 (s, 1H), 7.80 (s, 1H), 7.20 (s, |
| | cyclopropyl-7H-pyrrolo[2,3- | 1H), 4.71 (dt, J = 10.5, 4.8 Hz, 1H), 4.20-3.98 (m, 1H), |
| | d]pyrimidin-4-yl)-3- | 3.97-3.78 (m, 2H), 3.56 (t, J = 11.3 Hz, 1H), 3.33 (dd, J = |
| | methylpiperazine-1-carboxylate | 14.8, 6.5 Hz, 1H), 3.24-3.03 (m, 1H), 2.62 (s, 3H), 2.07- |
| | | 2.00 (m, 1H), 1.50 (s, 9H), 1.23 (d, J = 6.5 Hz, 3H), 1.04 |
| | | (dd, J = 8.2, 2.0 Hz, 2H), 0.91-0.86 (m, 1H), 0.81-0.74 |
| | | (m, 1H). |

### Example 16. Synthesis of tert-butyl (1S,6R)-5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate (Compound 408) and tert-butyl (1R,6S)-5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate (Compound 409)

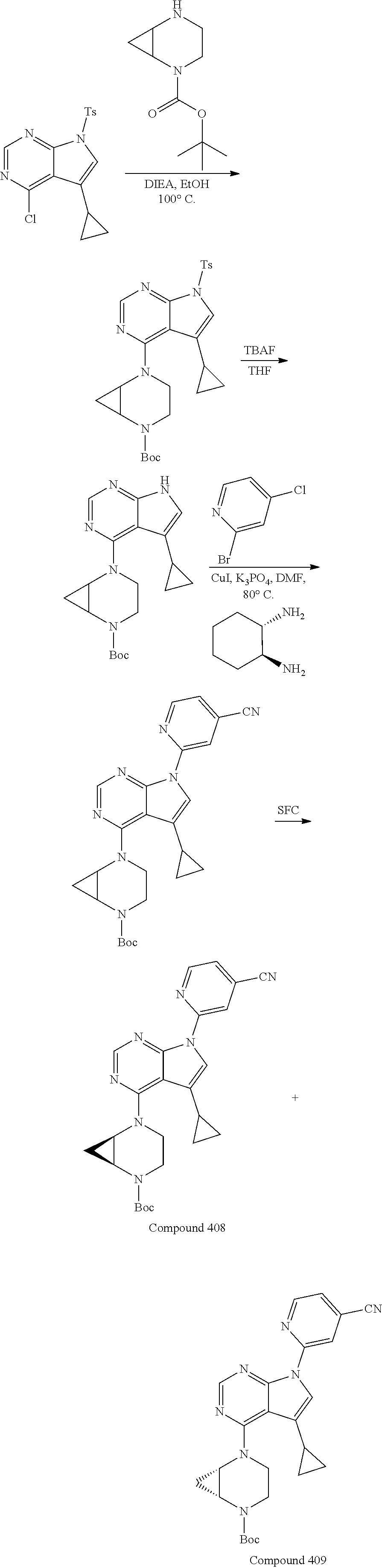

### Step 1. tert-Butyl 5-(5-cyclopropyl-7-tosyl-7H-pyrrolo[2,3-d]pyrimidin-4-yl)-2,5-diazabicyclo[4.1.0]heptane-2-carboxylate

To the solution of 4-chloro-5-cyclopropyl-7-tosyl-7H-pyrrolo[2,3-d]pyrimidine (170 mg, 0.87 mmol, prepared following the procedure of compound 192, step 1) in EtOH (5 mL) were added DIEA (0.14 mL, 0.87 mmol) and tert-butyl 2,5-diazabicyclo[4.1.0]heptane-2-carboxylate (140 mg, 0.72 mmol) respectively. The resulting reaction mixture was stirred at 100° C. under N₂ overnight. After removal of solvent, the residue was purified by flash column chromatography (silica gel, 0˜30%, ethyl acetate in petroleum ether) to afford tert-butyl 5-(5-cyclopropyl-7-tosyl-7H-pyrrolo[2,3-d]pyrimidin-4-yl)-2,5-diazabicyclo[4.1.0]heptane-2-carboxylate (140 mg, 45%) as white solid. LC/MS ESI (m/z): 510 (M+H)⁺.

### Step 2. tert-Butyl 5-(5-cyclopropyl-7H-pyrrolo[2,3-d]pyrimidin-4-yl)-2,5-diazabicyclo[4.1.0]heptane-2-carboxylate

To the solution of tert-butyl 5-(5-cyclopropyl-7-tosyl-7H-pyrrolo[2,3-d]pyrimidin-4-yl)-2,5-diazabicyclo[4.1.0]heptane-2-carboxylate (140 mg, 0.40 mmol) in THE (2 mL) were added TBAF (1.5 mL, 1.0M in THF). The resulting reaction mixture was stirred at room temperature under N₂ overnight. The reaction mixture was quenched with ice water and extracted with EtOAc twice, the combined organic layers were washed with brine, dried over Na₂SO₄, filtered, and concentrated under reduced pressure. The residue was purified by flash column chromatography (silica gel, 0˜50%, ethyl acetate in petroleum ether) to afford tert-butyl 5-(5-cyclopropyl-7H-pyrrolo[2,3-d]pyrimidin-4-yl)-2,5-diazabicyclo[4.1.0]heptane-2-carboxylate (90 mg, 64%) as a white solid. LC/MS ESI (m/z): 356 (M+H)⁺.

### Step 3. tert-Butyl 5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate

To the solution of tert-butyl 5-(5-cyclopropyl-7H-pyrrolo[2,3-d]pyrimidin-4-yl)-2,5-diazabicyclo[4.1.0]heptane-2-carboxylate (90 mg, 0.25 mmol) in DMF (6 mL) were added CuI (24 mg, 0.13 mmol), K₃PO₄ (160 mg, 0.75 mmol), trans-cyclohexane-1,2-diamine (9.0 mg, 0.080 mmol) and 2-bromoisonicotinonitrile (93 mg, 0.51 mmol) respectively. The resulting reaction mixture was stirred at 80° C. under N₂ overnight. After being cooled down to room temperature, the reaction mixture was partitioned between EtOAc and water, organic layer was separated, the aqueous layer was extracted with EtOAc twice, the combined organic layers were washed with brine, dried over Na₂SO₄, filtered and concentrated. The residue was purified by flash column chromatography (silica gel, 0-40%, ethyl acetate in petroleum ether), prep-HPLC (Gilson, C18, MeCN in water) to afford tert-butyl 5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate (60 mg, 53%) as white solid. LC/MS ESI (m/z): 458 (M+H)⁺.

### Step 4. tert-Butyl (1S,6R)-5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate and tert-butyl (1R,6S)-5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate

Preparative separation method: Instrument: Waters Thar 80 preparative SFC; Column: ChiralCel OJ, 250×21.2 mm I.D., 5 μm; Mobile phase: A for CO2 and B for MEOH+0.1% NH3H2O; Gradient: B 40%; Flow rate: 50 mL/min; Back pressure: 100 bar; Column temperature: 35° C.; Wavelength: 254 nm; Cycle-time: 7 min; Eluting time: 1.5H tert-Butyl 5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate (60 mg) was separated by SFC to give two isomers:

Peak 1: shorter retention time, 22 mg as white solid. LC/MS ESI (m/z): 458 (M+H)⁺. ¹H NMR (400 MHz, CDCl₃) δ 9.33 (d, J=3.2 Hz, 1H), 8.60-8.57 (m, 1H), 8.46-8.39 (m, 1H), 7.85-7.81 (m, 1H), 7.36-7.32 (m, 1H), 4.55-4.46 (m, 1H), 3.84-3.74 (m, 1H), 3.67-3.39 (m, 3H), 3.10-2.98 (m, 1H), 2.07-1.99 (m, 1H), 1.48 (s, 9H), 1.22-1.12 (m, 1H), 1.00-0.74 (m, 4H), 0.50-0.44 (m, 1H).

Peak 2: longer retention time, 24 mg as white solid. LC/MS ESI (m/z): 458 (M+H)⁺. ¹H NMR (400 MHz, CDCl₃) δ 9.37-9.29 (m, 1H), 8.59 (d, J=5.0 Hz, 1H), 8.45-8.40 (m, 1H), 7.85-7.81 (m, 1H), 7.34 (dd, J=5.0, 1.3 Hz, 1H), 4.55-4.46 (m, 1H), 3.84-3.75 (m, 1H), 3.67-3.60 (m, 0.5H), 3.56-3.46 (m, 1H), 3.45-3.36 (m, 1.5H), 3.10-2.98 (m, 1H), 2.07-2.00 (m, 1H), 1.48 (s, 9H), 1.21-1.13 (m, 1H), 0.99-0.90 (m, 2H), 0.82-0.68 (m, 2H), 0.50-0.44 (m, 1H).

### Example 17. Synthesis of ethyl 4-(5-(2-fluorophenyl)-7-(5-methoxypyridin-3-yl)-7H-pyrrolo[2,3-d]pyrimidin-4-yl)piperazine-1-carboxylate (Compound 1003)

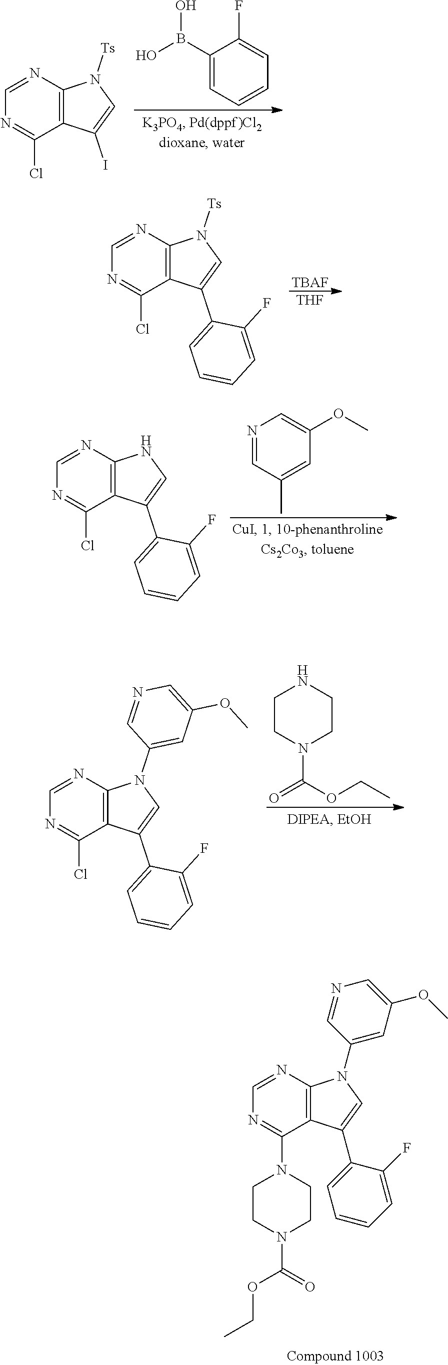

### Step 1. 4-Chloro-5-(2-fluorophenyl)-7-tosyl-7H-pyrrolo[2,3-d]pyrimidine

To a solution of 4-chloro-5-iodo-7-tosyl-7H-pyrrolo[2,3-d]pyrimidine (10 g, 23 mmol, prepared following the procedure of compound 192, step 1) in dioxane-H₂O (100 mL, v/v=5/1) were added (2-fluorophenyl)boronic acid (3.3 g, 23 mmol), K₃PO₄ (9.8 g, 46 mmol) and Pd(dppf)Cl₂ (1.7 g, 2.3 mmol). The resulting mixture was heated to 60° C. overnight. After being cooled down to room temperature, the reaction was filtered, filtrate was partitioned between EtOAc and water, organic layer was separated, the aqueous phase was extracted with EtOAc twice, the combined organic layers were washed with brine, dried over Na₂SO₄, filtered and concentrated, the residue was purified by flash chromatography (silica gel, 0˜50% EtOAc in petroleum ether) to give 4-chloro-5-(2-fluorophenyl)-7-tosyl-7H-pyrrolo[2,3-d]pyrimidine (7.0 g, 75% yield) as a solid. LC/MS ESI (m/z): 402 (M+H)⁺.

### Step 2. 4-Chloro-5-(2-fluorophenyl)-7H-pyrrolo[2,3-d]pyrimidine

To a 0° C. solution of 4-chloro-5-(2-fluorophenyl)-7-tosyl-7H-pyrrolo[2,3-d]pyrimidine (1.0 g, 2.50 mmol) in THE (5 mL) was added TBAF (7.5 mL, 1.0M in THF). The resulting mixture was stirred at the same temperature for 5 h. The reaction was quenched with ice water, extracted with EtOAc twice, the combined organic layers were washed with brine, dried over Na₂SO₄, filtered and concentrated. The residue was purified by flash column chromatography (silica gel, 0˜60% ethyl acetate in petroleum ether) to afford 4-chloro-5-(2-fluorophenyl)-7H-pyrrolo[2,3-d]pyrimidine (530 mg, 85%) LC/MS ESI (m/z): 248 (M+H)⁺.

### Step 3. 4-Chloro-5-(2-fluorophenyl)-7-(5-methoxypyridin-3-yl)-7H-pyrrolo[2,3-d]pyrimidine

To a solution of 4-chloro-5-(2-fluorophenyl)-7H-pyrrolo[2,3-d]pyrimidine (530 mg, 2.1 mmol) in toluene (20 mL) were added 3-iodo-5-methoxypyridine (600 mg, 2.6 mmol), CuI (81 mg, 0.40 mmol), 1,10-phenanthroline (77 mg, 0.40 mmol) and Cs₂CO₃ (2.1 g, 6.4 mmol). The resulting mixture was heated to 110° C. under N₂ overnight. After being cooled down to room temperature, the reaction was partitioned between EtOAc and water, organic layer was separated, aqueous layer was extracted with EtOAc twice, the combined organic layers were washed with brine, dried over Na₂SO₄, filtered and concentrated, the residue was purified by flash column chromatography (silica gel, 0˜30%, ethyl acetate in petroleum ether) to afford 4-chloro-5-(2-fluorophenyl)-7-(5-methoxypyridin-3-yl)-7H-pyrrolo[2,3-d]pyrimidine (400 mg, 53%) as a yellow solid. LC/MS ESI (m/z): 355 (M+H)⁺.

### Step 4. Ethyl 4-(5-(2-fluorophenyl)-7-(5-methoxypyridin-3-yl)-7H-pyrrolo[2,3-d]pyrimidin-4-yl)piperazine-1-carboxylate

To a solution of 4-chloro-5-(2-fluorophenyl)-7-(5-methoxypyridin-3-yl)-7H-pyrrolo[2,3-d]pyrimidine (100 mg, 0.30 mmol) in EtOH (10 mL) were added ethyl piperazine-1-carboxylate (63 mg, 0.30 mmol) and DIPEA (110 mg, 0.90 mmol). The resulting mixture was heated to 100° C. under N₂ overnight. After being cooled down to room temperature, solvent was removed and the residue was purified by flash column chromatography (silica gel, 0˜30%, ethyl acetate in petroleum ether) to afford ethyl 4-(5-(2-fluorophenyl)-7-(5-methoxypyridin-3-yl)-7H-pyrrolo[2,3-d]pyrimidin-4-yl)piperazine-1-carboxylate (50 mg, 37%) as a white solid, which was further purified by prep-HPLC to afford 22.8 mg of white solid. LC/MS ESI (m/z): 477 (M+H)⁺. ¹H NMR (400 MHz, DMSO-d6) δ 8.77 (d, J=2.0 Hz, 1H), 8.47 (s, 1H), 8.34 (d, J=2.6 Hz, 1H), 8.08 (s, 1H), 7.94 (t, J=2.3 Hz, 1H), 7.60-7.54 (m, 1H), 7.52-7.46 (m, 1H), 7.41-7.36 (m, 2H), 4.00 (q, J=7.1 Hz, 2H), 3.92 (s, 3H), 3.24-3.20 (m, 4H), 3.16-3.09 (m, 4H), 1.15 (t, J=7.1 Hz, 3H).

The following compound was prepared by the procedure analogous to the synthesis of compound 1003 from the corresponding amine.

| Cmpd | | |
|---|---|---|
| No. | Chemical Name | LCMS and ¹H NMR |
| 103 | tert-butyl 4-(5-(2-fluorophenyl)- | LC/MS ESI (m/z): 505 (M + H)⁺. ¹H NMR (400 MHz, |
| | 7-(5-methoxypyridin-3-yl)-7H- | DMSO-d6) δ 8.77 (d, J = 2.0 Hz, 1H), 8.47 (s, 1H), 8.34 (d, |
| | pyrrolo[2,3-d]pyrimidin-4- | J = 2.6 Hz, 1H), 8.08 (s, 1H), 7.94 (t, J = 2.3 Hz, 1H), 7.60- |
| | yl)piperazine-1-carboxylate | 7.54 (m, 1H), 7.52-7.46 (m, 1H), 7.42-7.36 (m, 2H), |
| | | 3.92 (s, 3H), 3.22-3.18 (m, 4H), 3.12-3.04 (m, 4H), 1.37 |
| | | (s, 9H). |

### Example 18. Synthesis of tert-butyl 4-(7-(3-chloro-4-fluorophenyl)-5-(pyridin-2-yl)-7H-pyrrolo[2,3-d]pyrimidin-4-yl)piperazine-1-carboxylate (Compound 151)

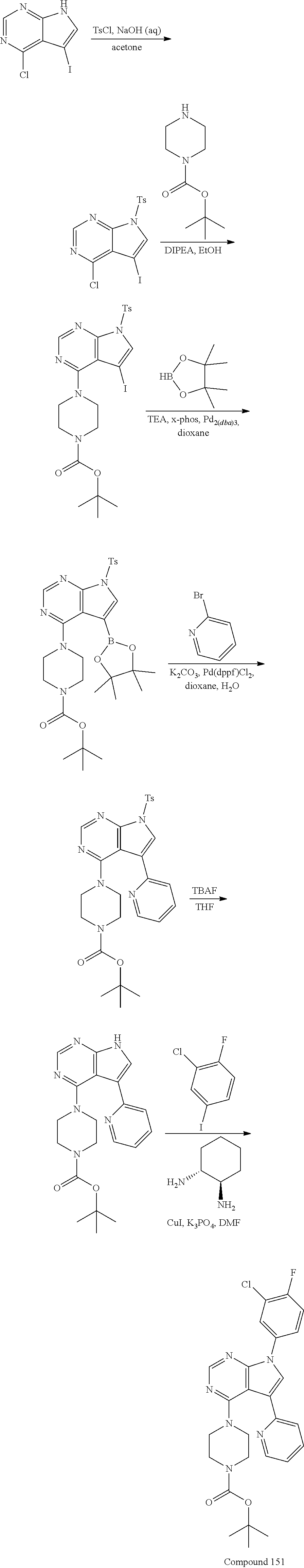

### Step 1. 4-Chloro-5-iodo-7-tosyl-7H-pyrrolo[2,3-d]pyrimidine

To a 0° C. solution of 4-chloro-5-iodo-7H-pyrrolo[2,3-d]pyrimidine (200 g, 0.71 mol) and 4-methylbenzene-1-sulfonyl chloride (180 g, 0.93 mol) in acetone (2 L) was added dropwise 2.0M NaOH (0.53 L). After addition, the reaction was allowed to warm up to room temperature and stirred for another 3 hours. The precipitate was collected by filtration and washed with water twice and dried in vacuo to afford 4-chloro-5-iodo-7-tosyl-7H-pyrrolo[2,3-d]pyrimidine (300 g, 95%) as an off white solid. LC/MS ESI (m/z): 434 (M+H)⁺.

### Step 2. tert-Butyl 4-(5-iodo-7-tosyl-7H-pyrrolo[2,3-d]pyrimidin-4-yl)piperazine-1-carboxylate

A mixture of 4-chloro-5-iodo-7-tosyl-7H-pyrrolo[2,3-d]pyrimidine (4.6 g, 11 mmol), tert-butyl piperazine-1-carboxylate (2.2 g, 12 mmol) and DIPEA (2.8 mL, 16 mmol) in EtOH (20 mL) was stirred at 100° C. overnight. After being cooled down to r.t, solvent was removed and the residue was purified by flash column chromatography to provide tert-butyl 4-(5-iodo-7-tosyl-7H-pyrrolo[2,3-d]pyrimidin-4-yl)piperazine-1-carboxylate (5.5 g, 89%) as a white solid. LC/MS ESI (m/z): 584 (M+H)⁺.

### Step 3. tert-Butyl 4-(5-(4,4,5,5-tetramethyl-1,3,2-dioxaborolan-2-yl)-7-tosyl-7H-pyrrolo[2,3-d]pyrimidin-4-yl)piperazine-1-carboxylate

To a solution of tert-butyl 4-(5-iodo-7-tosyl-7H-pyrrolo[2,3-d]pyrimidin-4-yl)piperazine-1-carboxylate (300 mg, 0.51 mmol) in dioxane (5 mL) were added 4,4,5,5-tetramethyl-1,3,2-dioxaborolane (0.30 mL, 2.0 mmol), TEA (0.35 mL, 2.5 mmol), X-Phos (25 mg, 0.052 mmol) and Pd₂(dba)₃ (47 mg, 0.052 mmol). The resulting mixture was stirred at 95° C. overnight. After being cooled down to room temperature. The reaction was quenched with water, extracted with DCM twice, the combined organic layers were washed with brine, dried over Na₂SO₄, filtered and concentrated to afford crude tert-butyl 4-(5-(4,4,5,5-tetramethyl-1,3,2-dioxaborolan-2-yl)-7-tosyl-7H-pyrrolo[2,3-d]pyrimidin-4-yl)piperazine-1-carboxylate (300 mg, 99%) as a yellow oil. LC/MS ESI (m/z): 584 (M+H)⁺.

### Step 4. tert-Butyl 4-(5-(pyridin-2-yl)-7-tosyl-7H-pyrrolo[2,3-d]pyrimidin-4-yl)piperazine-1-carboxylate

To a solution of tert-butyl 4-(5-(4,4,5,5-tetramethyl-1,3,2-dioxaborolan-2-yl)-7-tosyl-7H-pyrrolo[2,3-d]pyrimidin-4-yl)piperazine-1-carboxylate (300 mg, 0.51 mmol) in dioxane (5 mL) and H₂O (1 mL) were added 2-bromopyridine (0.96 mL, 1.0 mmol), K₂CO₃ (360 mg, 2.5 mmol) and Pd(dppf)Cl₂ (38 mg, 0.052 mmol). The resulting mixture was heated to 90° C. overnight. After being cooled down to room temperature, solvent was removed and the residue was purified by flash column chromatography (silica gel, 0˜60%, ethyl acetate in petroleum ether) to afford tert-butyl 4-(5-(pyridin-2-yl)-7-tosyl-7H-pyrrolo[2,3-d]pyrimidin-4-yl)piperazine-1-carboxylate (180 mg, 65%) as a white solid. LC/MS ESI (m/z): 535 (M+H)⁺.

### Step 5. tert-Butyl 4-(5-(pyridin-2-yl)-7H-pyrrolo[2,3-d]pyrimidin-4-yl)piperazine-1-carboxylate

To a solution of tert-butyl 4-(5-(pyridin-2-yl)-7-tosyl-7H-pyrrolo[2,3-d]pyrimidin-4-yl)piperazine-1-carboxylate (180 mg, 0.33 mmol) in THE (2 mL) was added TBAF (2.0 mL, 1.0M in THF). The resulting mixture was stirred at room temperature overnight. The reaction was quenched with water, extracted with EtOAc twice, the combined organic layers were washed with brine, dried over Na₂SO₄, filtered and concentrated. The residue was purified by flash column chromatography (silica gel, 0-10%, methanol in dichloromethane) to afford tert-butyl 4-(5-(pyridin-2-yl)-7H-pyrrolo[2,3-d]pyrimidin-4-yl)piperazine-1-carboxylate (120 mg, 93%) as a white solid. LC/MS ESI (m/z): 381 (M+H)⁺.

### Step 6. tert-Butyl 4-(7-(3-chloro-4-fluorophenyl)-5-(pyridin-2-yl)-7H-pyrrolo[2,3-d]pyrimidin-4-yl)piperazine-1-carboxylate

To a solution of tert-butyl 4-(5-(pyridin-2-yl)-7H-pyrrolo[2,3-d]pyrimidin-4-yl)piperazine-1-carboxylate (120 mg, 0.31 mmol) in DMF (10 mL) was added 2-chloro-1-fluoro-4-iodobenzene (0.048 mL, 0.37 mmol), trans-cyclohexane-1,2-diamine (11 mg, 0.095 mmol), CuI (18 mg, 0.095 mmol) and K₃PO₄ (200 mg, 0.94 mmol). The resulting mixture was heated to 120° C. overnight. After being cooled down to room temperature. The reaction was quenched with water, extracted with EtOAc twice, the combined organic layers were washed with brine, dried over Na₂SO₄, filtered and concentrated. The residue was purified by flash column chromatography (silica gel, 0˜50%, ethyl acetate in petroleum ether) to afford tert-butyl 4-(7-(3-chloro-4-fluorophenyl)-5-(pyridin-2-yl)-7H-pyrrolo[2,3-d]pyrimidin-4-yl)piperazine-1-carboxylate (130 mg, 80%) as a white solid. LC/MS ESI (m/z): 509 (M+H)⁺. ¹H NMR (400 MHz, CDCl₃) δ 8.69 (d, J=4.3 Hz, 1H), 8.52 (s, 1H), 7.85 (dd, J=6.4, 2.6 Hz, 1H), 7.79 (td, J=7.7, 1.8 Hz, 1H), 7.67 (s, 1H), 7.67-7.60 (m, 2H), 7.34-7.23 (m, 2H), 3.35 (d, J=17.5 Hz, 8H), 1.44 (s, 9H).

The following compound was prepared by analogous procedures to the synthesis of compound 151 from the corresponding amines and aryl halides.

| Cmpd | | |
|---|---|---|
| No. | Chemical Name | LCMS and ¹H NMR |
| 154 | tert-butyl (S)-4-(7-(3-chloro-4- | LC/MS ESI (m/z): 523 (M + H)⁺. ¹H NMR (400 MHz, |
| | fluorophenyl)-5-(pyridin-2-yl)- | DMSO-d6) δ 8.69 (d, J = 4.1 Hz, 1H), 8.51 (s, 1H), 7.85 |
| | 7H-pyrrolo[2,3-d]pyrimidin-4- | (dd, J = 6.4, 2.6 Hz, 1H), 7.79 (m, 1H), 7.67-7.63 (m, |
| | yl)-3-methylpiperazine-1- | 2H), 7.60 (m, 1H), 7.34-7.24 (m, 2H), 4.39-4.20 (m, |
| | carboxylate | 1H), 3.95-3.79 (m, 1H), 3.64-3.52 (m, 2H), 3.17 (m, |
| | | 1H), 3.08-2.71 (m, 2H), 1.44 (s, 9H), 1.08-0.99 (m, 3H). |

### Example 19. Synthesis of ethyl 4-(7-(3-chloro-4-fluorophenyl)-5-(pyridin-2-yl)-7H-pyrrolo[2,3-d]pyrimidin-4-yl)piperazine-1-carboxylate (Compound 152)

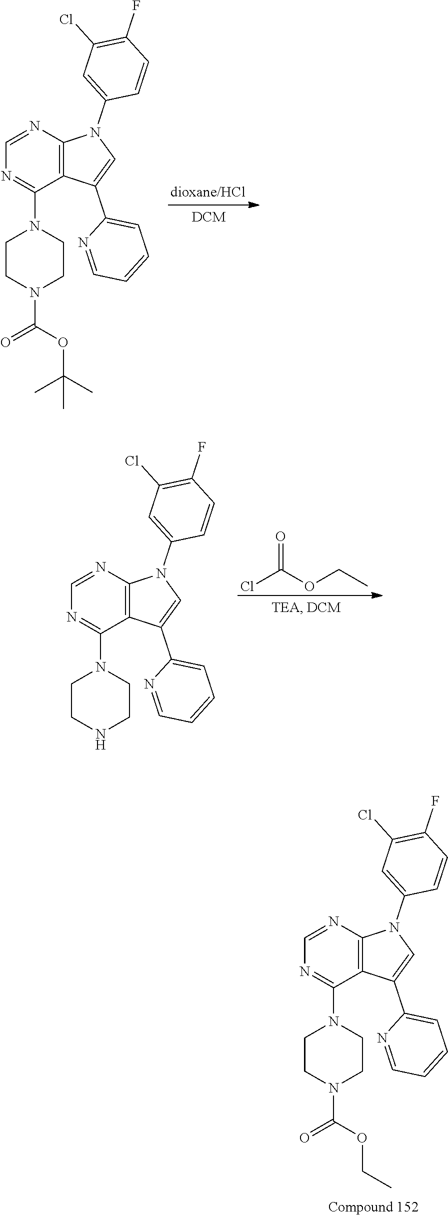

### Step 1. 7-(3-Chloro-4-fluorophenyl)-4-(piperazin-1-yl)-5-(pyridin-2-yl)-7H-pyrrolo[2,3-d]pyrimidine

To a solution of tert-butyl 4-(7-(3-chloro-4-fluorophenyl)-5-(pyridin-2-yl)-7H-pyrrolo[2,3-d]pyrimidin-4-yl)piperazine-1-carboxylate (100 mg, 0.19 mmol, prepared following the procedure of compound 151 in DCM (3 mL) was added HCl (3.0 mL, 4.0M in dioxane). The resulting mixture was stirred at room temperature for 3 h. After removal of solvent, the residue was diluted with DCM, washed with NaHCO₃ (aq.), the organic layer was extracted with DCM twice, the combined organic layers were washed with brine, dried over Na₂SO₄, filtered and concentrated. The residue was used in the next step directly, 7-(3-chloro-4-fluorophenyl)-4-(piperazin-1-yl)-5-(pyridin-2-yl)-7H-pyrrolo[2,3-d]pyrimidine. LC/MS ESI (m/z): 409 (M+H)⁺.

### Step 2. Ethyl 4-(7-(3-chloro-4-fluorophenyl)-5-(pyridin-2-yl)-7H-pyrrolo[2,3-d]pyrimidin-4-yl)piperazine-1-carboxylate

To a 0° C. solution of 7-(3-chloro-4-fluorophenyl)-4-(piperazin-1-yl)-5-(pyridin-2-yl)-7H-pyrrolo[2,3-d]pyrimidine (80 mg, 0.19 mmol) in DCM (3 mL) were added dropwise ethyl carbonochloridate (0.040 mL, 0.39 mmol) and TEA (0.080 mL, 0.58 mmol). The resulting mixture was stirred at room temperature overnight. The reaction was quenched with water, extracted with DCM twice, the combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford ethyl 4-(7-(3-chloro-4-fluorophenyl)-5-(pyridin-2-yl)-7H-pyrrolo[2,3-d]pyrimidin-4-yl)piperazine-1-carboxylate (28 mg, 30%) as a white solid. LC/MS ESI (m/z): 481 (M+H)⁺. ¹H NMR (400 MHz, CDCl₃) δ 8.72-8.66 (m, 1H), 8.53 (s, 1H), 7.85 (dd, J=6.4, 2.6 Hz, 1H), 7.79 (td, J=7.7, 1.8 Hz, 1H), 7.67-7.59 (m, 3H), 7.34-7.24 (m, 2H), 4.12 (q, J=7.1 Hz, 2H), 3.38 (s, 8H), 1.24 (t, J=7.1 Hz, 3H).

### Example 20. Synthesis of tert-butyl 4-(7-(5-chloropyridin-3-yl)-5-cyclopropyl-7H-pyrrolo[2,3-d]pyrimidin-4-yl)-3-methylpiperazine-1-carboxylate (Compound 164)

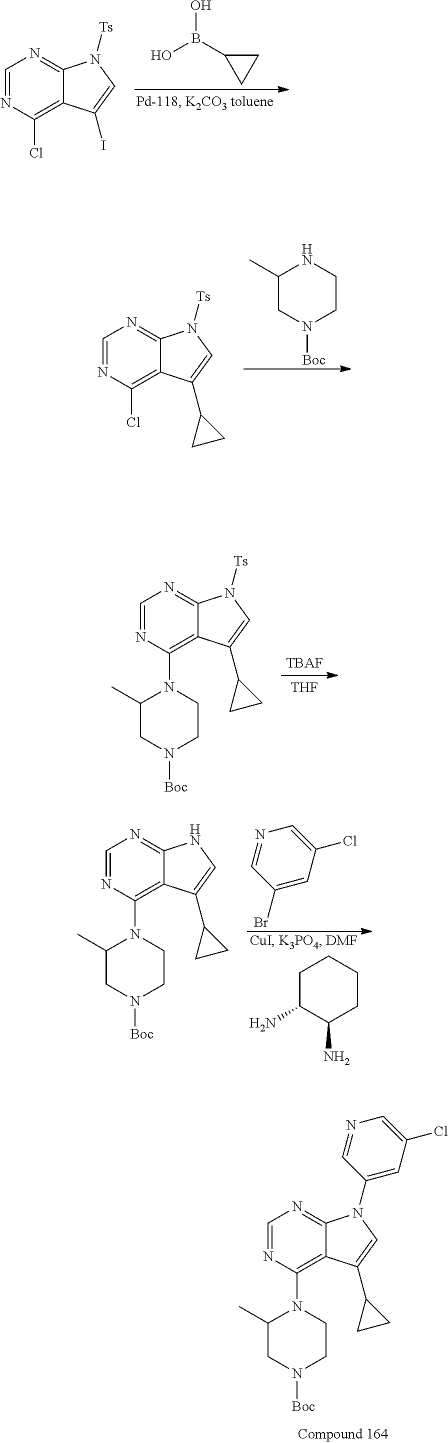

### Step 1. 4-Chloro-5-cyclopropyl-7-tosyl-7H-pyrrolo[2,3-d]pyrimidine

To a solution of 4-chloro-5-iodo-7-tosyl-7H-pyrrolo[2,3-d]pyrimidine (5.8 g, 13 mmol, prepared following the procedure of compound 151, step 1) toluene (50 mL) were added cyclopropylboronic acid (1.1 g, 13 mmol), K₂CO₃ (24 g, 170 mmol) and Pd-118 (880 mg, 1.3 mmol). The resulting mixture was heated to 80° C. overnight. After being cooled down to room temperature, solvent was filtered. The filtrate was concentrated and purified by flash column chromatography (silica gel, 0-30%, ethyl acetate in petroleum ether) to give 4-chloro-5-cyclopropyl-7-tosyl-7H-pyrrolo[2,3-d]pyrimidine (3.6 g, 77% yield) as a solid. LC/MS ESI (m/z): 348 (M+H)⁺.

### Step 2. tert-Butyl 4-(5-cyclopropyl-7-tosyl-7H-pyrrolo[2,3-d]pyrimidin-4-yl)-3-methylpiperazine-1-carboxylate

The mixture of 4-chloro-5-cyclopropyl-7-tosyl-7H-pyrrolo[2,3-d]pyrimidine (500 mg, 1.4 mmol) and tert-butyl 3-methylpiperazine-1-carboxylate (1200 mg, 5.7 mmol) was heated to 150° C. for 3 h. After being cooled down to room temperature, the reaction mixture was purified by flash column chromatography (silica gel, 0-30%, ethyl acetate in petroleum ether) to afford tert-butyl 4-(5-cyclopropyl-7-tosyl-7H-pyrrolo[2,3-d]pyrimidin-4-yl)-3-methylpiperazine-1-carboxylate (450 mg, 61%) as a white solid. LC/MS ESI (m/z): 512 (M+H)⁺.

### Step 3. tert-Butyl 4-(5-cyclopropyl-7H-pyrrolo[2,3-d]pyrimidin-4-yl)-3-methylpiperazine-1-carboxylate

To a solution of tert-butyl 4-(5-cyclopropyl-7-tosyl-7H-pyrrolo[2,3-d]pyrimidin-4-yl)-3-methylpiperazine-1-carboxylate (450 mg, 0.88 mmol) in THE (5 mL) was added TBAF (5.3 mL, 1.0M in THF). The resulting mixture was stirred at room temperature overnight. The reaction was quenched with water, extracted with EtOAc twice, the combined organic layers were washed with brine, dried over Na₂SO₄, filtered and concentrated. The residue was purified by flash column chromatography (silica gel, 0˜60%, ethyl acetate in petroleum ether) to afford tert-butyl 4-(5-cyclopropyl-7H-pyrrolo[2,3-d]pyrimidin-4-yl)-3-methylpiperazine-1-carboxylate (240 mg, 76%) as a yellow solid. LC/MS ESI (m/z): 358 (M+H)⁺.

### Step 4. tert-Butyl 4-(7-(5-chloropyridin-3-yl)-5-cyclopropyl-7H-pyrrolo[2,3-d]pyrimidin-4-yl)-3-methylpiperazine-1-carboxylate

To a solution of tert-butyl 4-(5-cyclopropyl-7H-pyrrolo[2,3-d]pyrimidin-4-yl)-3-methylpiperazine-1-carboxylate (120 mg, 0.33 mmol) in DMF (5 mL) were added 3-bromo-5-chloropyridine (78 mg, 0.40 mmol), trans-cyclohexane-1,2-diamine (12 mg, 0.10 mmol), CuI (19 mg, 0.10 mmol) and K₃PO₄ (210 mg, 1.0 mmol). The resulting mixture was heated to 120° C. overnight. After being cooled down to room temperature, the reaction was diluted with water, extracted with EtOAc twice, the combined organic layers were washed with brine, dried over Na₂SO₄, filtered, and concentrated. The residue was purified by flash column chromatography (silica gel, 0˜40%, ethyl acetate in petroleum ether) to give crude product which was further purified by prep-HPLC to afford tert-butyl 4-(7-(5-chloropyridin-3-yl)-5-cyclopropyl-7H-pyrrolo[2,3-d]pyrimidin-4-yl)-3-methylpiperazine-1-carboxylate (53 mg, 33%) as a white solid. LC/MS ESI (m/z): 469 (M+H)⁺. ¹H NMR (400 MHz, CDCl₃) δ 8.81 (d, J=2.1 Hz, 1H), 8.52 (d, J=2.0 Hz, 1H), 8.44 (s, 1H), 8.26 (t, J=2.1 Hz, 1H), 6.94 (s, 1H), 4.76 (s, 1H), 4.19-3.79 (m, 3H), 3.56 (t, J=12.0 Hz, 1H), 3.37-3.03 (m, 2H), 2.06-1.99 (m, 1H), 1.50 (s, 9H), 1.25 (d, J=6.5 Hz, 3H), 1.04 (dd, J=8.1, 1.7 Hz, 2H), 0.85-0.77 (m, 1H), 0.76-0.67 (m, 1H).

The following compounds were prepared by analogous procedures to the synthesis of compound 164 from the corresponding amines and aryl halides.

| Cmpd | | |
|---|---|---|
| No. | Chemical Name | LCMS and ¹H NMR |
| 127 | tert-butyl (S)-4-(5-cyclopropyl- | LC/MS ESI (m/z): 452 (M + H)⁺. ¹H NMR (400 MHz, |
| | 7-(3-fluorophenyl)-7H- | DMSO-d6) δ 8.37 (s, 1H), 7.83 (dt, J = 11.0, 2.2 Hz, 1H), |
| | pyrrolo[2,3-d]pyrimidin-4-yl)-3- | 7.76 (dd, J = 8.2, 1.3 Hz, 1H), 7.58-7.52 (m, 2H), 7.18 (td, |
| | methylpiperazine-1-carboxylate | J = 8.4, 1.9 Hz, 1H), 4.71-4.62 (m, 1H), 4.04-3.84 (m, |
| | | 2H), 3.78 (d, J = 13.0 Hz, 1H), 3.45-3.37 (m, 1H), 3.33- |
| | | 3.14 (m, 2H), 2.07-1.99 (m, 1H), 1.44 (s, 9H), 1.13 (d, J = |
| | | 6.5 Hz, 3H), 1.01-0.95 (m, 2H), 0.92-0.88 (m, 1H), 0.83- |
| | | 0.77 (m, 1H). |
| 128 | tert-butyl (S)-4-(5-cyclopropyl- | LC/MS ESI (m/z): 452 (M + H)⁺. ¹H NMR (400 MHz, |
| | 7-(4-fluorophenyl)-7H- | DMSO) δ 8.32 (s, 1H), 7.84-7.79 (m, 2H), 7.45 (s, 1H), |
| | pyrrolo[2,3-d]pyrimidin-4-yl)-3- | 7.39-7.34 (m, 2H), 4.72-4.63 (m, 1H), 4.03-3.86 (m, |
| | methylpiperazine-1-carboxylate | 2H), 3.77 (d, J = 13.6 Hz, 1H), 3.47-3.38 (m, 1H), 3.32- |
| | | 3.24 (m, 2H), 2.06-1.99 (m, 1H), 1.43 (s, 9H), 1.13 (d, J = |
| | | 6.5 Hz, 3H), 1.00-0.94 (m, 2H), 0.90-0.85 (m, 1H), 0.81- |
| | | 0.75 (m, 1H). |
| 129 | tert-butyl (S)-4-(5-cyclopropyl- | LC/MS ESI (m/z): 502 (M + H)⁺. ¹H NMR (400 MHz, |
| | 7-(3,5-dichlorophenyl)-7H- | DMSO-d6) δ 8.41 (s, 1H), 8.13 (d, J = 1.8 Hz, 2H), 7.64 (s, |
| | pyrrolo[2,3-d]pyrimidin-4-yl)-3- | 1H), 7.56 (t, J = 1.8 Hz, 1H), 4.70-4.60 (m, 1H), 4.05- |
| | methylpiperazine-1-carboxylate | 3.86 (m, 2H), 3.78 (d, J = 13.2 Hz, 1H), 3.47-3.37 (m, |
| | | 1H), 3.33-3.20 (m, 2H), 2.05-1.98 (m, 1H), 1.43 (s, 9H), |
| | | 1.13 (d, J = 6.5 Hz, 3H), 1.01-0.95 (m, 2H), 0.94-0.89 |
| | | (m, 1H), 0.84-0.79 (m, 1H). |
| 106 | tert-butyl (S)-4-(5-cyclopropyl- | LC/MS ESI (m/z): 465 (M + H)⁺. ¹H NMR (400 MHz, |
| | 7-(5-methoxypyridin-3-yl)-7H- | CDCl₃) δ 8.44 (d, J = 6.3 Hz, 2H), 8.29 (s, 1H), 7.76 (s, |
| | pyrrolo[2,3-d]pyrimidin-4-yl)-3- | 1H), 6.95 (s, 1H), 4.76 (s, 1H), 4.19-3.78 (m, 6H), 3.64- |
| | methylpiperazine-1-carboxylate | 3.48 (m, 1H), 3.41-3.03 (m, 2H), 2.10-1.98 (m, 1H), |
| | | 1.50 (s, 9H), 1.24 (d, J = 6.7 Hz, 3H), 1.03 (d, J = 8.1 Hz, |
| | | 2H), 0.87-0.77 (m, 1H), 0.76-0.68 (m, 1H). |
| 150 | tert-butyl (S)-4-(5-cyclopropyl- | LC/MS ESI (m/z): 449 (M + H)⁺. ¹H NMR (400 MHz, |
| | 7-(5-methylpyridin-3-yl)-7H- | DMSO) δ 8.87 (d, J = 2.4 Hz, 1H), 8.38 (d, J = 1.1 Hz, 1H), |
| | pyrrolo[2,3-d]pyrimidin-4-yl)-3- | 8.35 (s, 1H), 8.09 (s, 1H), 7.54 (s, 1H), 4.72-4.65 (m, 1H), |
| | methylpiperazine-1-carboxylate | 4.05-3.88 (m, 2H), 3.78 (d, J = 12.9 Hz, 1H), 3.48-3.36 |
| | | (m, 2H), 3.15-3.00 (m, 1H), 2.39 (s, 3H), 2.07-2.00 (m, |
| | | 1H), 1.44 (s, 9H), 1.14 (d, J = 6.5 Hz, 3H), 1.01-0.96 (m, |
| | | 2H), 0.90-0.85 (m, 1H), 0.82-0.76 (m, 1H). |
| 165 | tert-butyl (2R,5S)-4-(5- | LC/MS ESI (m/z): 463 (M + H)⁺. ¹H NMR (400 MHz, |
| | cyclopropyl-7-(5-methylpyridin- | CD₃OD) δ 8.77 (s, 1H), 8.38 (s, 1H), 8.29 (s, 1H), 8.05 (s, |
| | 3-yl)-7H-pyrrolo[2,3- | 1H), 7.31 (d, J = 0.9 Hz, 1H), 5.00-4.91 (m, 1H), 4.44- |
| | d]pyrimidin-4-yl)-2,5- | 4.36 (m, 1H), 3.82-3.77 (m, 3H), 3.68-3.59 (m, 1H), |
| | dimethylpiperazine-1- | 2.46 (s, 3H), 2.11-2.03 (m, 1H), 1.50 (s, 9H), 1.17 (dd, J = |
| | carboxylate | 8.1, 6.8 Hz, 6H), 1.09-1.03 (m, 2H), 0.98-0.92 (m, 1H), |
| | | 0.72-0.67 (m, 1H). |
| 167 | tert-butyl (2R,5S)-4-(5- | LC/MS ESI (m/z): 517 (M + H)⁺. ¹H NMR (400 MHz, |
| | cyclopropyl-7-(5- | CD₃OD) δ 9.30 (d, J = 2.3 Hz, 1H), 8.83 (s, 1H), 8.70 (s, |
| | (trifluoromethyl)pyridin-3-yl)- | 1H), 8.35 (s, 1H), 7.47 (d, J = 0.9 Hz, 1H), 4.99-4.91 (m, |
| | 7H-pyrrolo[2,3-d]pyrimidin-4- | 1H), 4.44-4.35 (m, 1H), 3.83-3.77 (m, 3H), 3.69-3.59 |
| | yl)-2,5-dimethylpiperazine-1- | (m, 1H), 2.11-2.03 (m, 1H), 1.50 (s, 9H), 1.17 (dd, J = |
| | carboxylate | 6.7, 4.2 Hz, 6H), 1.10-1.04 (m, 2H), 1.01-0.95 (m, 1H), |
| | | 0.75-0.69 (m, 1H). |
| 168 | tert-butyl (2R,5S)-4-(5- | LC/MS ESI (m/z): 449 (M + H)⁺. ¹H NMR (400 MHz, |
| | cyclopropyl-7-(pyridin-3-yl)- | CD₃OD) δ 8.98 (s, 1H), 8.53 (s, 1H), 8.30 (s, 1H), 8.23 |
| | 7H-pyrrolo[2,3-d]pyrimidin-4- | (ddd, J = 8.3, 2.4, 1.3 Hz, 1H), 7.60 (dd, J = 8.2, 4.8 Hz, |
| | yl)-2,5-dimethylpiperazine-1- | 1H), 7.34 (d, J = 0.9 Hz, 1H), 4.99-4.92 (m, 1H), 4.44- |
| | carboxylate | 4.36 (m, 1H), 3.82-3.77 (m, 3H), 3.69-3.59 (m, 1H), |
| | | 2.10-2.04 (m, 1H), 1.50 (s, 9H), 1.17 (dd, J = 8.0, 6.9 Hz, |
| | | 6H), 1.08-1.03 (m, 2H), 0.98-0.92 (m, 1H), 0.72-0.67 |
| | | (m, 1H). |

### Example 21. Synthesis of tert-butyl (S)-4-(7-(3-chlorophenyl)-5-(3-fluoropyrazin-2-yl)-7H-pyrrolo[2,3-d]pyrimidin-4-yl)-3-methylpiperazine-1-carboxylate (Compound 267)

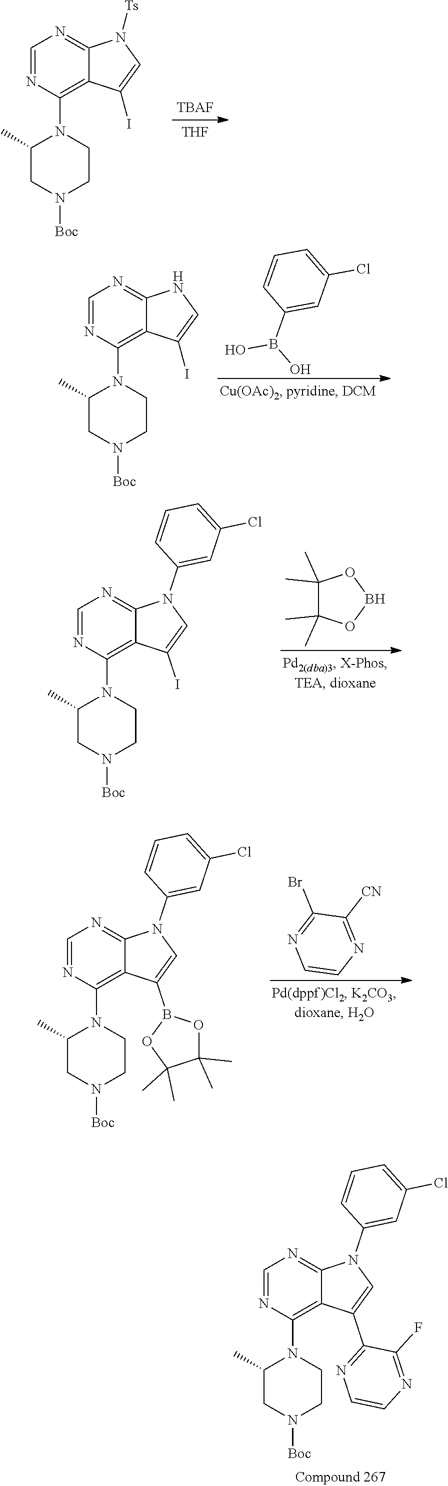

### Step 1. tert-Butyl (S)-4-(5-iodo-7H-pyrrolo[2,3-d]pyrimidin-4-yl)-3-methylpiperazine-1-carboxylate

A solution of tert-butyl (S)-4-(5-iodo-7-tosyl-7H-pyrrolo[2,3-d]pyrimidin-4-yl)-3-methylpiperazine-1-carboxylate (1.0 g, 1.6 mmol, prepared following the procedures of step 1 of compound 259 synthesis) in THE (5 mL) at 0° C. was treated with TBAF (6.7 mL, 1.0M in THF). The resulting mixture was stirred at 0° C. for 3 h. The reaction was quenched with water, extracted twice with EtOAc. The combined organic layers were washed with brine, dried over Na₂SO₄, filtered and concentrated. The residue was purified by flash column chromatography (silica gel, 0˜70%, ethyl acetate in petroleum ether) to afford tert-butyl (S)-4-(5-iodo-7H-pyrrolo[2,3-d]pyrimidin-4-yl)-3-methylpiperazine-1-carboxylate (0.55 g, 74%) as a yellow solid. LC/MS ESI (m/z): 444 (M+H)⁺.

### Step 2. tert-Butyl (S)-4-(7-(3-chlorophenyl)-5-iodo-7H-pyrrolo[2,3-d]pyrimidin-4-yl)-3-methylpiperazine-1-carboxylate

To a solution of tert-butyl (S)-4-(5-iodo-7H-pyrrolo[2,3-d]pyrimidin-4-yl)-3-methylpiperazine-1-carboxylate (550 mg, 1.2 mmol) in DCM (15 mL) were added (3-chlorophenyl)boronic acid (390 mg, 2.4 mmol), Cu(OAc)₂ (670 mg, 3.7 mmol), pyridine (0.60 mL, 7.4 mmol) and 4 A molecular sieves (400 mg). The resulting mixture was stirred at 40° C. under an O₂ atmosphere overnight. After cooling in an ice-water bath, the reaction was quenched with aq. NH₄OH (2 mL) and filtered. The filtrate was extracted twice with DCM. The combined organic layers were dried over Na₂SO₄, filtered and concentrated. The residue was purified by flash column chromatography (silica gel, 0˜30%, ethyl acetate in petroleum ether) to afford tert-butyl (S)-4-(7-(3-chlorophenyl)-5-iodo-7H-pyrrolo[2,3-d]pyrimidin-4-yl)-3-methylpiperazine-1-carboxylate (240 mg, 35%) as a yellow solid. LC/MS ESI (m/z): 554 (M+H)⁺.

### Step 3. tert-Butyl (S)-4-(7-(3-chlorophenyl)-5-(4,4,5,5-tetramethyl-1,3,2-dioxaborolan-2-yl)-7H-pyrrolo[2,3-d]pyrimidin-4-yl)-3-methylpiperazine-1-carboxylate

To a solution of tert-butyl (S)-4-(7-(3-chlorophenyl)-5-iodo-7H-pyrrolo[2,3-d]pyrimidin-4-yl)-3-methylpiperazine-1-carboxylate (240 mg, 0.43 mmol) in dioxane (10 mL) were added 4,4,5,5-tetramethyl-1,3,2-dioxaborolane (0.25 mL, 1.7 mmol), TEA (0.30 mL, 2.1 mmol), X-Phos (21 mg, 0.043 mmol) and Pd₂(dba)₃ (40 mg, 0.043 mmol). The resulting mixture was stirred at 95° C. overnight. The reaction was quenched with water and extracted twice with DCM. The combined organic layers were washed with brine, dried over Na₂SO₄, filtered and concentrated to afford crude tert-butyl (S)-4-(7-(3-chlorophenyl)-5-(4,4,5,5-tetramethyl-1,3,2-dioxaborolan-2-yl)-7H-pyrrolo[2,3-d]pyrimidin-4-yl)-3-methylpiperazine-1-carboxylate as a yellow oil, which was used in the next step directly. LC/MS ESI (m/z): 554 (M+H)⁺.

### Step 4. tert-Butyl (S)-4-(7-(3-chlorophenyl)-5-(3-fluoropyrazin-2-yl)-7H-pyrrolo[2,3-d]pyrimidin-4-yl)-3-methylpiperazine-1-carboxylate

To a solution of tert-butyl (S)-4-(7-(3-chlorophenyl)-5-(4,4,5,5-tetramethyl-1,3,2-dioxaborolan-2-yl)-7H-pyrrolo[2,3-d]pyrimidin-4-yl)-3-methylpiperazine-1-carboxylate (120 mg, 0.21 mmol) in dioxane (5 mL) and H₂O (1 mL) were added 2-bromo-3-fluoropyrazine (77 mg, 0.43 mmol), K₂CO₃ (150 mg, 1.0 mmol) and Pd(dppf)Cl₂ (16 mg, 0.022 mmol). The resulting mixture was heated at 90° C. overnight. After cooling to room temperature, solvent was removed and the residue was purified by flash column chromatography (silica gel, 0˜50%, ethyl acetate in petroleum ether) to give crude product which was further purified by prep-HPLC to afford tert-butyl (S)-4-(7-(3-chlorophenyl)-5-(3-fluoropyrazin-2-yl)-7H-pyrrolo[2,3-d]pyrimidin-4-yl)-3-methylpiperazine-1-carboxylate (59 mg, 51%) as a white solid. LC/MS ESI (m/z): 524 (M+H)⁺. ¹H NMR (400 MHz, CDCl₃) δ 8.59 (dd, J=3.9, 2.6 Hz, 1H), 8.52 (s, 1H), 8.21-8.14 (m, 1H), 7.77 (t, J=1.9 Hz, 1H), 7.74 (s, 1H), 7.66 (d, J=8.0 Hz, 1H), 7.49 (t, J=8.0 Hz, 1H), 7.43-7.38 (m, 1H), 4.62-4.24 (m, 1H), 3.88-3.72 (m, 1H), 3.67 (d, J=13.2 Hz, 1H), 3.60-3.39 (m, 1H), 3.18-2.64 (m, 3H), 1.44 (s, 9H), 1.13 (d, J=13.8 Hz, 3H).

The following compound was prepared by a procedure analogous to the synthesis of compound 267 using the corresponding aryl halide.

| Cmpd | | |
|---|---|---|
| No. | Chemical Name | LCMS and ¹H NMR |
| 271 | tert-butyl (S)-4-(7-(3- | LC/MS ESI (m/z): 534 (M + H)⁺. ¹H NMR (400 MHz, |
| | chlorophenyl)-5-(3-ethylpyrazin- | CDCl₃) δ 8.58 (d, J = 2.5 Hz, 1H), 8.54 (s, 1H), 8.52 (d, J = |
| | 2-yl)-7H-pyrrolo[2,3- | 2.4 Hz, 1H), 7.79 (t, J = 1.8 Hz, 1H), 7.68 (dd, J = 8.0, 1.0 |
| | d]pyrimidin-4-yl)-3- | Hz, 1H), 7.47 (dd, J = 13.9, 5.8 Hz, 2H), 7.41-7.33 (m, |
| | methylpiperazine-1-carboxylate | 1H), 4.09 (d, J = 75.1 Hz, 1H), 3.73 (s, 1H), 3.59-3.35 (m, |
| | | 2H), 3.01 (s, 1H), 2.92-2.67 (m, 3H), 2.54 (s, 1H), 1.43 (s, |
| | | 9H), 1.18 (t, J = 7.5 Hz, 3H), 0.98 (s, 3H). |

### Example 22. Synthesis of tert-butyl 4-(3-(2-fluorophenyl)-1-(pyrimidin-5-yl)-1H-pyrrolo[3,2-c]pyridin-4-yl)piperazine-1-carboxylate (Compound 171)

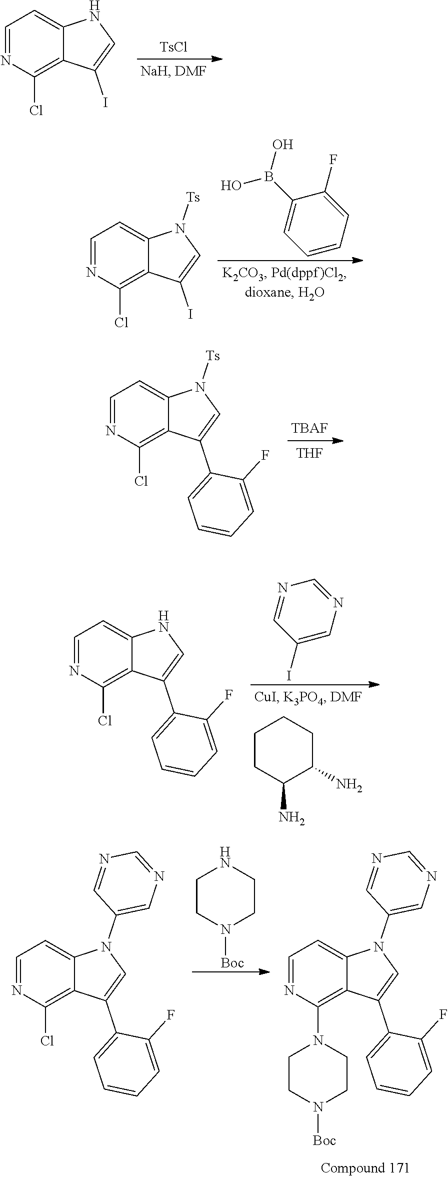

### Step 1. 4-Chloro-3-iodo-1-tosyl-1H-pyrrolo[3,2-c]pyridine

To a 0° C. suspension of NaH (400 mg, 10 mmol, 60 wt % in mineral oil) in anhydrous DMF (30 mL) was added 4-chloro-3-iodo-1H-pyrrolo[3,2-c]pyridine (1.4 g, 5.0 mmol, prepared following the procedure for compound 418, step 1) in portions, followed by 4-methylbenzenesulfonyl chloride (1.1 g, 6.0 mmol) in portions. The resulting mixture was stirred at room temperature overnight. The reaction was poured into ice water, extracted with EtOAc twice, the combined organic layers washed with brine, dried over Na₂SO₄, filtered and concentrated. The residue was purified by flash column chromatography (silica gel, 0˜30%, ethyl acetate in petroleum ether) to afford 4-chloro-3-iodo-1-tosyl-1H-pyrrolo[3,2-c]pyridine (1.7 g, 80%) as a light yellow solid. LC/MS ESI (m/z): 433 (M+H)⁺.

### Step 2. 4-Chloro-3-(2-fluorophenyl)-1-tosyl-1H-pyrrolo[3,2-c]pyridine

To a solution of 4-chloro-3-iodo-1-tosyl-1H-pyrrolo[3,2-c]pyridine (920 mg, 2.1 mmol) in dioxane (10 mL) and H₂O (2 mL) was added (2-fluorophenyl)boronic acid (300 mg, 2.1 mmol), K₂CO₃ (880 mg, 6.3 mmol) and Pd(dppf)Cl₂ (160 mg, 0.21 mmol). The resulting mixture was heated at 90° C. overnight. After being cooled to room temperature, the solvent was removed in vacuo and the residue was purified by flash column chromatography (silica gel, 0˜30%, ethyl acetate in petroleum ether) to afford 4-chloro-3-(2-fluorophenyl)-1-tosyl-1H-pyrrolo[3,2-c]pyridine (680 mg, 79%) as a white solid. LC/MS ESI (m/z): 401 (M+H)⁺.

### Step 3. 4-Chloro-3-(2-fluorophenyl)-1H-pyrrolo[3,2-c]pyridine

To a solution of 4-chloro-3-(2-fluorophenyl)-1-tosyl-1H-pyrrolo[3,2-c]pyridine (680 mg, 1.7 mmol) in THE (5 mL) was added TBAF (10 mL, 1.0M in THF). The resulting mixture was stirred at room temperature overnight. The reaction was quenched with water, extracted with EtOAc twice, the combined organic layers washed with brine, dried over Na₂SO₄, filtered and concentrated. The residue was purified by flash column chromatography (silica gel, 0˜50%, ethyl acetate in petroleum ether) to afford 4-chloro-3-(2-fluorophenyl)-1H-pyrrolo[3,2-c]pyridine (410 mg, 97%) as a white solid. LC/MS ESI (m/z): 247 (M+H)⁺.

### Step 4. 4-Chloro-3-(2-fluorophenyl)-1-(pyrimidin-5-yl)-1H-pyrrolo[3,2-c]pyridine

To a solution of 4-chloro-3-(2-fluorophenyl)-1H-pyrrolo[3,2-c]pyridine (410 mg, 1.6 mmol) in DMF (15 mL) were added 5-iodopyrimidine (690 mg, 3.3 mmol), trans-cyclohexane-1,2-diamine (57 mg, 0.49 mmol), CuI (320 mg, 1.6 mmol) and K₃PO₄ (1.1 mg, 4.9 mmol). The resulting mixture was heated at 120° C. overnight. After cooling to room temperature, the reaction was quenched with water, extracted with EtOAc twice, the combined organic layers washed with brine, dried over Na₂SO₄, filtered and concentrated. The residue was purified by flash column chromatography (silica gel, 0˜50%, ethyl acetate in petroleum ether) to afford 4-chloro-3-(2-fluorophenyl)-1-(pyrimidin-5-yl)-1H-pyrrolo[3,2-c]pyridine (70 mg, 13%) as a yellow oil. LC/MS ESI (m/z): 325 (M+H)⁺.

### Step 5. tert-Butyl 4-(3-(2-fluorophenyl)-1-(pyrimidin-5-yl)-1H-pyrrolo[3,2-c]pyridin-4-yl)piperazine-1-carboxylate

4-chloro-3-(2-fluorophenyl)-1-(pyrimidin-5-yl)-1H-pyrrolo[3,2-c]pyridine (70 mg, 0.21 mmol) and tert-butyl piperazine-1-carboxylate (200 mg, 1.0 mmol). was heated at 150° C. for 3 h. After cooling to room temperature, the reaction mixture was purified by flash column chromatography (silica gel, 0˜60%, ethyl acetate in petroleum ether) to give crude product which was further purified by prep-HPLC to afford tert-butyl 4-(3-(2-fluorophenyl)-1-(pyrimidin-5-yl)-1H-pyrrolo[3,2-c]pyridin-4-yl)piperazine-1-carboxylate (2.5 mg, 2.0%) as a white solid. LC/MS ESI (m/z): 475 (M+H)⁺. ¹H NMR (400 MHz, CDCl₃) δ 9.29 (s, 1H), 9.00 (s, 2H), 8.12 (d, J=5.9 Hz, 1H), 7.54 (td, J=7.7, 1.8 Hz, 1H), 7.41-7.36 (m, 1H), 7.35 (d, J=0.7 Hz, 1H), 7.25-7.17 (m, 2H), 7.08 (d, J=5.9 Hz, 1H), 3.05 (s, 8H), 1.43 (s, 9H).

### Example 23. Synthesis of 1-fluoro-2-methylpropan-2-yl 4-(5-(2-fluorophenyl)-7-(pyridin-3-yl)-7H-pyrrolo[2,3-d]pyrimidin-4-yl)piperazine-1-carboxylate (Compound 172)

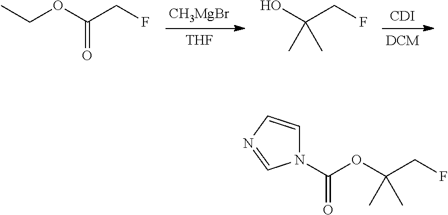

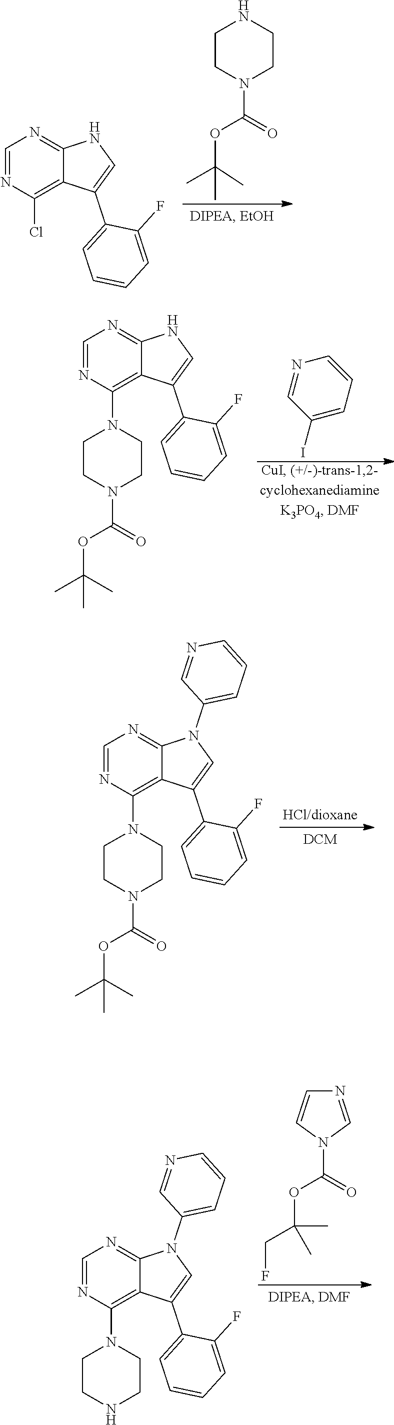

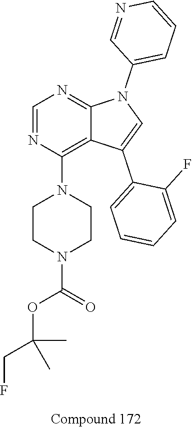 [SMILES: CC(C)(CF)OC(=O)N1CCN(c2ncnc3c2c(-c2ccccc2F)cn3-c2cccnc2)CC1]

### Step 1. I-Fluoro-2-methylpropan-2-ol

To a −60° C. solution of ethyl 2-fluoroacetate (10 g, 94 mmol) in dry THE (30 mL) under N2 was added CH₃MgBr (210 mL, 1.0 M in THF, 210 mmol) dropwise. The mixture was kept at the same temperature for 1 h and then at 0° C. for 4 h. It was quenched by addition of 50 mL of ice water, and conc. HCl (18 mL) and solid NaCl (10 g) in sequence. The mixture was extracted with DCM. The organic layer was dried over Na₂SO₄ overnight, and concentrated by rotary evaporation, keeping the bath temperature below 25° C. The residue was purified by distillation under normal pressure to afford 1-fluoro-2-methylpropan-2-ol (3.5 g, 40%) as a colorless liquid (bp˜92° C.). ¹H NMR (400 MHz, CDCl₃) δ 4.20 (d, J=47.7 Hz, 2H), 1.25 (t, J=2.6 Hz, 6H), hydroxyl group proton exchanged.

### Step 2. I-Fluoro-2-methylpropan-2-yl 1H-imidazole-1-carboxylate

To CDI (530 mg, 3.3 mmol) in dry DCM (8 mL) under N₂ was added 1-fluoro-2-methylpropan-2-ol (300 mg, 3.3 mmol) dropwise. The mixture was stirred at rt overnight. It was then diluted with DCM, washed with water and brine, dried over Na₂SO₄ and concentrated to provide 1-fluoro-2-methylpropan-2-yl 1H-imidazole-1-carboxylate (290 mg, 47%) as a colorless oil. LC/MS ESI (m/z): 187 (M+H)⁺.

### Step 3. tert-Butyl 4-(5-(2-fluorophenyl)-7H-pyrrolo[2,3-d]pyrimidin-4-yl)piperazine-1-carboxylate

A mixture of 4-chloro-5-(2-fluorophenyl)-7H-pyrrolo[2,3-d]pyrimidine (580 mg, 2.3 mmol), tert-butyl piperazine-1-carboxylate (520 mg, 2.8 mmol, prepared following the procedure of compound 1003, first two steps) and DIPEA (1.6 mL, 9.7 mmol) in EtOH (10 mL) was stirred at 100° C. overnight. The solvents were removed by rotary evaporation. The residue was purified by flash column chromatography (silica gel, 0˜50% EtOAc in petroleum ether, then 100% DCM) to provide tert-butyl 4-(5-(2-fluorophenyl)-7H-pyrrolo[2,3-d]pyrimidin-4-yl)piperazine-1-carboxylate (560 mg, 60%) as a light yellow solid. LC/MS ESI (m/z): 398 (M+H)⁺.

### Step 4. tert-Butyl 4-(5-(2-fluorophenyl)-7-(pyridin-3-yl)-7H-pyrrolo[2,3-d]pyrimidin-4-yl)piperazine-1-carboxylate

To tert-butyl 4-(5-(2-fluorophenyl)-7H-pyrrolo[2,3-d]pyrimidin-4-yl)piperazine-1-carboxylate (200 mg, 0.50 mmol) and 3-iodopyridine (410 mg, 2.0 mmol) in DMF (8 mL) was added (±)-trans-1,2-cyclohexanediamine (57 mg, 0.50 mmol), and K₃PO₄ (320 mg, 1.5 mmol) and CuI (96 mg, 0.50 mmol) and the mixture was stirred at 100° C. under N₂ overnight. It was then diluted with EtOAc and filtered. The filtrate was washed with LiCl (5% aq.) and brine, dried over Na₂SO₄ and concentrated. The residue was purified by flash column chromatography (silica gel, 0˜50% EtOAc in petroleum ether, then 100% DCM) to provide tert-butyl 4-(5-(2-fluorophenyl)-7-(pyridin-3-yl)-7H-pyrrolo[2,3-d]pyrimidin-4-yl)piperazine-1-carboxylate (170 mg, ˜70% purity, 48%) as a light yellow solid. LC/MS ESI (m/z): 475 (M+H)⁺.

### Step 5. 5-(2-Fluorophenyl)-4-(piperazin-1-yl)-7-(pyridin-3-yl)-7H-pyrrolo[2,3-d]pyrimidine

To tert-butyl 4-(5-(2-fluorophenyl)-7-(pyridin-3-yl)-7H-pyrrolo[2,3-d]pyrimidin-4-yl)piperazine-1-carboxylate (170 mg, 0.24 mmol) in DCM (2 mL) was added HCl/dioxane (3.0 mL, 4.0 M). The mixture was stirred at rt overnight. The solvent was evaporated, the residue was diluted with DCM, washed with NaHCO₃ (aq.), brine, dried over Na₂SO₄ and concentrated to provide 5-(2-fluorophenyl)-4-(piperazin-1-yl)-7-(pyridin-3-yl)-7H-pyrrolo[2,3-d]pyrimidine (130 mg, 99%) as a solid. LC/MS ESI (m/z): 375 (M+H)⁺.

### Step 6. I-Fluoro-2-methylpropan-2-yl 4-(5-(2-fluorophenyl)-7-(pyridin-3-yl)-7H-pyrrolo[2,3-d]pyrimidin-4-yl)piperazine-1-carboxylate

A mixture of 5-(2-fluorophenyl)-4-(piperazin-1-yl)-7-(pyridin-3-yl)-7H-pyrrolo[2,3-d]pyrimidine (120 mg, 0.32 mmol), 1-fluoro-2-methylpropan-2-yl 1H-imidazole-1-carboxylate (180 mg, 0.97 mmol) and DIPEA (0.26 mL, 1.6 mmol) in DMF (5 mL) was stirred at 80° C. under N₂ for 40 h. The mixture was diluted with EtOAc, washed with LiCl (5% aq.), brine and was then concentrated. The residue was purified by flash column chromatography (silica gel, 0˜50% EtOAc in petroleum ether) to give 80 mg of the crude product, which was further purified by prep-HPLC to afford 1-fluoro-2-methylpropan-2-yl 4-(5-(2-fluorophenyl)-7-(pyridin-3-yl)-7H-pyrrolo[2,3-d]pyrimidin-4-yl)piperazine-1-carboxylate (38 mg, 24%) as a white solid. LC/MS ESI (m/z): 493 (M+H)⁺. ¹H NMR (400 MHz, CDCl₃) δ 8.97 (d, J=1.9 Hz, 1H), 8.64 (d, J=4.0 Hz, 1H), 8.52 (s, 1H), 8.22 (ddd, J=8.2, 2.6, 1.5 Hz, 1H), 7.54-7.45 (m, 3H), 7.37 (tdd, J=7.1, 5.1, 1.8 Hz, 1H), 7.28-7.19 (m, 2H), 4.45 (d, J=47.5 Hz, 2H), 3.27 (d, J=29.6 Hz, 8H), 1.45 (d, J=2.2 Hz, 6H).

The following compound was prepared by a synthetic procedure analogous to that described for compound 172, except using 5-iodopyrimidine in step 4.

| Cmpd | | |
|---|---|---|
| No. | Chemical Name | LCMS and ¹H NMR |
| 173 | 1-fluoro-2-methylpropan-2-yl 4- | LC/MS ESI (m/z): 494 (M + H)⁺. ¹H NMR (400 MHz, |
| | (5-(2-fluorophenyl)-7- | CDCl₃) δ 9.27 (s, 2H), 9.22 (s, 1H), 8.52 (s, 1H), 7.51- |
| | (pyrimidin-5-yl)-7H-pyrrolo[2,3- | 7.44 (m, 2H), 7.39 (tdd, J = 7.2, 5.1, 1.8 Hz, 1H), 7.30- |
| | d]pyrimidin-4-yl)piperazine-1- | 7.18 (m, 2H), 4.45 (d, J = 47.5 Hz, 2H), 3.28 (d, J = 31.3 |
| | carboxylate | Hz, 8H), 1.45 (d, J = 2.2 Hz, 6H). |

### Example 24. Synthesis of 1,1,1-trifluoro-2-methylpropan-2-yl (2R,5S)-4-(5-(2-fluorophenyl)-7-(1-methyl-1H-pyrazol-4-yl)-7H-pyrrolo[2,3-d]pyrimidin-4-yl)-2,5-dimethylpiperazine-1-carboxylate (Compound 186)

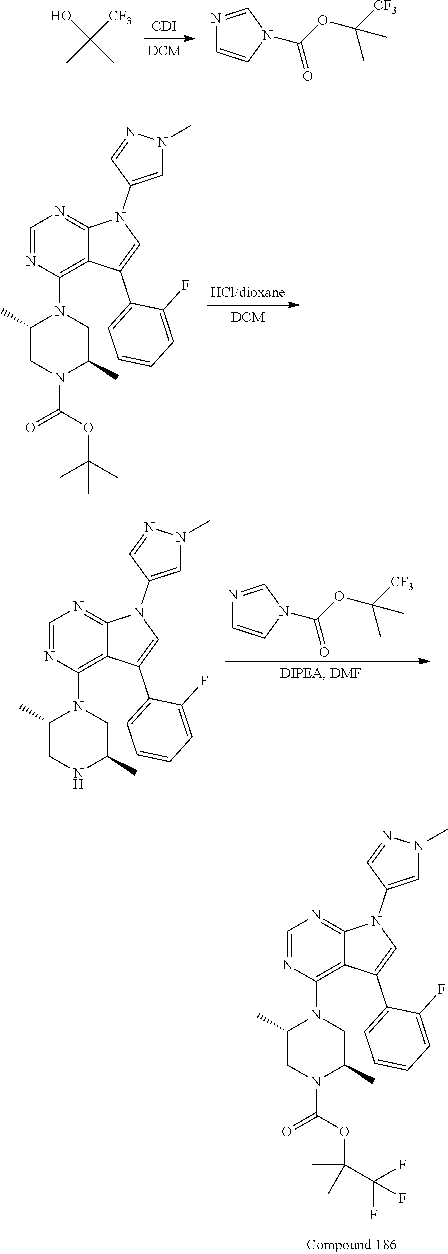

### Step 1. 1,1,1-Trifluoro-2-methylpropan-2-yl 1H-imidazole-1-carboxylate

To a solution of 1,1,1-trifluoro-2-methylpropan-2-ol (500 mg, 3.9 mmol) in DCM (10 mL) was added di(1H-imidazol-1-yl)methanone (630 mg, 3.9 mmol) and the resulting mixture was stirred at room temperature overnight. The reaction was quenched with water, extracted with DCM twice, the combined organic layers washed with brine, dried over Na₂SO₄, filtered and concentrated to afford 1,1,1-trifluoro-2-methylpropan-2-yl 1H-imidazole-1-carboxylate (640 mg, 73%) as a white solid. LC/MS ESI (m/z): 223 (M+H)⁺.

### Step 2. 4-((2S,5R)-2,5-Dimethylpiperazin-1-yl)-5-(2-fluorophenyl)-7-(I-methyl-1H-pyrazol-4-yl)-7H-pyrrolo[2,3-d]pyrimidine

To a solution of tert-butyl (2R,5S)-4-(5-(2-fluorophenyl)-7-(1-methyl-1H-pyrazol-4-yl)-7H-pyrrolo[2,3-d]pyrimidin-4-yl)-2,5-dimethylpiperazine-1-carboxylate (110 mg, 0.21 mmol, prepared following a similar procedure of compound 134 in DCM (2 mL) was added HCl/dioxane (0.50 mL, 4.0M). The resulting mixture was stirred at room temperature for 3 h. After removal of solvent, the residue was dissolved in DCM, washed with NaHCO₃ (aq), the organic layer was separated, aqueous layer was extracted with DCM twice, the combined organic layers were washed with brine, dried over Na₂SO₄, filtered and concentrated. The residue was used in the next step directly. 4-((2S,5R)-2,5-dimethylpiperazin-1-yl)-5-(2-fluorophenyl)-7-(1-methyl-1H-pyrazol-4-yl)-7H-pyrrolo[2,3-d]pyrimidine. LC/MS ESI (m/z): 406 (M+H)⁺.

### Step 3. 1,1,1-Trifluoro-2-methylpropan-2-yl (2R,5S)-4-(5-(2-fluorophenyl)-7-(1-methyl-1H-pyrazol-4-yl)-7H-pyrrolo[2,3-d]pyrimidin-4-yl)-2,5-dimethylpiperazine-1-carboxylate

To a solution of 4-((2S,5R)-2,5-dimethylpiperazin-1-yl)-5-(2-fluorophenyl)-7-(1-methyl-1H-pyrazol-4-yl)-7H-pyrrolo[2,3-d]pyrimidine (30 mg, 0.074 mmol) in DMF (3 mL) was added 1,1,1-trifluoro-2-methylpropan-2-yl 1H-imidazole-1-carboxylate (20 mg, 0.089 mmol) and DIPEA (0.040 mL, 0.22 mmol). The resulting mixture was stirred at 80° C. for 2 d under N₂. The reaction was quenched with water, extracted with EtOAc twice, the combined organic layers washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford 1,1,1-trifluoro-2-methylpropan-2-yl (2R,5S)-4-(5-(2-fluorophenyl)-7-(1-methyl-1H-pyrazol-4-yl)-7H-pyrrolo[2,3-d]pyrimidin-4-yl)-2,5-dimethylpiperazine-1-carboxylate (14 mg, 33%) as a white solid. LC/MS ESI (m/z): 560 (M+H)⁺. ¹H NMR (400 MHz, CDCl₃) δ 8.51 (s, 1H), 8.12 (s, 1H), 7.79 (d, J=0.5 Hz, 1H), 7.47 (td, J=7.5, 1.6 Hz, 1H), 7.39-7.31 (m, 2H), 7.26-7.16 (m, 2H), 4.27-4.03 (m, 2H), 4.00 (s, 3H), 3.39 (d, J=13.1 Hz, 1H), 3.32-3.16 (m, 2H), 3.03-2.82 (m, 1H), 1.71-1.59 (m, 6H), 1.14-1.06 (m, 3H), 1.01-0.91 (m, 3H).

The following compounds were prepared by the procedures analogous to the synthesis of compound 186 from the corresponding boronic acids and alcohols.

| Cmpd | | |
|---|---|---|
| No. | Chemical Name | LCMS and ¹H NMR |
| 185 | 1-fluoro-2-methylpropan-2-yl | LC/MS ESI (m/z): 524 (M + H)⁺. ¹H NMR (400 MHz, |
| | (2R,5S)-4-(5-(2-fluorophenyl)-7- | CDCl₃) δ 8.51 (s, 1H), 8.12 (s, 1H), 7.79 (s, 1H), 7.47 (td, |
| | (1-methyl-1H-pyrazol-4-yl)-7H- | J = 7.5, 1.7 Hz, 1H), 7.38-7.33 (m, 2H), 7.25-7.17 (m, |
| | pyrrolo[2,3-d]pyrimidin-4-yl)- | 2H), 4.44 (d, J = 47.3 Hz, 2H), 4.29-4.08 (m, 2H), 4.00 (s, |
| | 2,5-dimethylpiperazine-1- | 3H), 3.45-3.17 (m, 3H), 3.04-2.82 (m, 1H), 1.45 (s, 6H), |
| | carboxylate | 1.09 (d, J = 6.8 Hz, 3H), 0.95 (d, J = 6.6 Hz, 3H). |
| 191 | 1-fluoro-2-methylpropan-2-yl | LC/MS ESI (m/z): 492 (M + H)⁺. ¹H NMR (400 MHz, |
| | (S)-3-methyl-4-(7-(1-methyl-1H- | CDCl₃) δ 8.51 (s, 1H), 8.11 (s, 1H), 7.78 (s, 1H), 7.50 (d, |
| | pyrazol-4-yl)-5-phenyl-7H- | J = 7.2 Hz, 2H), 7.48-7.41 (m, 2H), 7.38-7.32 (m, 1H), |
| | pyrrolo[2,3-d]pyrimidin-4- | 7.25 (s, 1H), 4.43 (d, J = 47.6 Hz, 2H), 4.22-4.12 (m, 1H), |
| | yl)piperazine-1-carboxylate | 4.00 (s, 3H), 3.91-3.67 (m, 1H), 3.60-3.37 (m, 2H), 3.12 |
| | | (td, J = 13.0, 3.3 Hz, 1H), 2.98-2.60 (m, 2H), 1.44 (d, J = |
| | | 1.8 Hz, 6H), 0.98 (d, J = 6.6 Hz, 3H). |

### Example 25. Synthesis of tert-butyl (S)-4-(7-(3-methoxyphenyl)-5-(pyridin-2-yl)-7H-pyrrolo[2,3-d]pyrimidin-4-yl)-3-methylpiperazine-1-carboxylate (Compound 278)

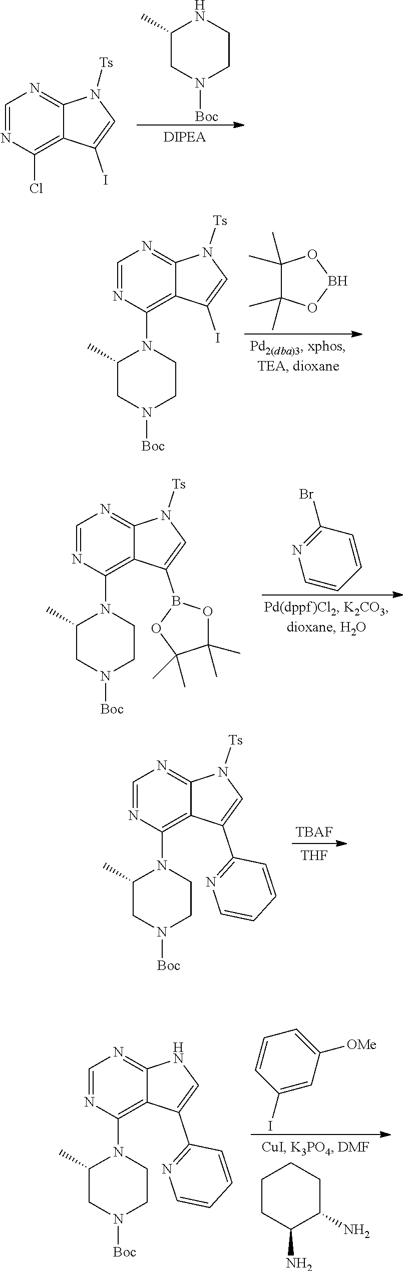

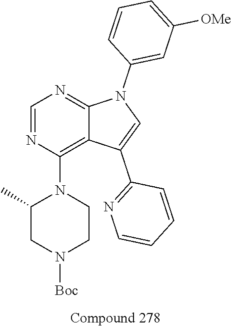 [SMILES: COc1cccc(-n2cc(-c3ccccn3)c3c(N4CCN(C(=O)OC(C)(C)C)C[C@@H]4C)ncnc32)c1]

### Step 1. tert-Butyl (S)-4-(5-iodo-7-tosyl-7H-pyrrolo[2,3-d]pyrimidin-4-yl)-3-methylpiperazine-1-carboxylate

To a solution of 4-chloro-5-iodo-7-tosyl-7H-pyrrolo[2,3-d]pyrimidine (5.0 g, 12 mmol) in DIPEA (15 mL) was added tert-butyl (S)-3-methylpiperazine-1-carboxylate (5.8 g, 29 mmol). The resulting mixture was heated to 140° C. for 1.5 h. After being cooled down to room temperature, solvent was removed and the residue was purified by flash column chromatography (silica gel, 0˜30%, ethyl acetate in petroleum ether) to afford tert-butyl (S)-4-(5-iodo-7-tosyl-7H-pyrrolo[2,3-d]pyrimidin-4-yl)-3-methyl piperazine-1-carboxylate (5.8 g, 84%) as a yellow solid. LC/MS ESI (m/z): 598 (M+H)⁺.

### Step 2. tert-Butyl (S)-3-methyl-4-(5-(4,4,5,5-tetramethyl-1,3,2-dioxaborolane-2-yl)-7-tosyl-7H-pyrrolo[2,3-d]pyrimidin-4-yl)piperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (1.0 g, 1.6 mmol) in dioxane (15 mL) were added 4,4,5,5-tetramethyl-1,3,2-dioxaborolane (0.97 mL, 6.6 mmol), TEA (1.2 mL, 8.3 mmol), X-Phos (0.080 g, 0.16 mmol) and Pd₂(dba)₃ (0.15 g, 0.16 mmol). The resulting mixture was stirred at 95° C. overnight. After being cooled down to room temperature. The reaction was quenched with water, extracted with DCM twice, the combined organic layers were washed with brine, dried over Na₂SO₄, filtered and concentrated to afford crude tert-butyl (S)-3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate as a yellow oil, which was used in the next step directly. LC/MS ESI (m/z): 598 (M+H)⁺.

### Step 3. tert-Butyl (S)-3-methyl-4-(5-(pyridin-2-yl)-7-tosyl-7H-pyrrolo[2,3-d]pyrimidin-4-yl)piperazine-1-carboxylate

To a solution of tert-butyl (S)-3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate (1.0 g, 1.6 mmol) in dioxane (15 mL) and H₂O (3 mL) were added 2-bromopyridine (0.32 mL, 3.3 mmol), K₂CO₃ (1.2 g, 8.3 mmol) and Pd(dppf)Cl₂ (0.12 g, 0.16 mmol). The resulting mixture was heated to 90° C. overnight. After being cooled down to room temperature, solvent was removed and the residue was purified by flash column chromatography (silica gel, 0˜50%, ethyl acetate in petroleum ether) to afford tert-butyl (S)-3-methyl-4-(5-(pyridin-2-yl)-7-tosyl-7H-pyrrolo[2,3-d]pyrimidin-4-yl)piperazine-1-carboxylate (0.69 g, 75%) as a yellow solid. LC/MS ESI (m/z): 549 (M+H)⁺.

### Step 4. tert-Butyl (S)-3-methyl-4-(5-(pyridin-2-yl)-7H-pyrrolo[2,3-d]pyrimidin-4-yl)piperazine-1-carboxylate

To a solution of tert-butyl (S)-3-methyl-4-(5-(pyridin-2-yl)-7-tosyl-7H-pyrrolo[2,3-d]pyrimidin-4-yl)piperazine-1-carboxylate (690 mg, 1.3 mmol) in THE (5 mL) was added TBAF (5.0 mL, 1.0M in THF). The resulting mixture was stirred at room temperature overnight. The reaction was quenched with water, extracted with EtOAc twice, the combined organic layers were washed with brine, dried over Na₂SO₄, filtered and concentrated. The residue was purified by flash column chromatography (silica gel, 0˜10%, methanol in dichloromethane) to afford tert-butyl (S)-3-methyl-4-(5-(pyridin-2-yl)-7H-pyrrolo[2,3-d]pyrimidin-4-yl)piperazine-1-carboxylate (450 mg, 90%) as a white solid. LC/MS ESI (m/z): 395 (M+H)⁺.

### Step 5. tert-Butyl (S)-4-(7-(3-methoxyphenyl)-5-(pyridin-2-yl)-7H-pyrrolo[2,3-d]pyrimidin-4-yl)-3-methylpiperazine-1-carboxylate

To a solution of tert-butyl (S)-3-methyl-4-(5-(pyridin-2-yl)-7H-pyrrolo[2,3-d]pyrimidin-4-yl)piperazine-1-carboxylate (120 mg, 0.30 mmol) in DMF (10 mL) were added 1-iodo-3-methoxybenzene (0.054 mL, 0.45 mmol), trans-cyclohexane-1,2-diamine (10 mg, 0.091 mmol), CuI (58 mg, 0.30 mmol) and K₃PO₄ (190 mg, 0.91 mmol). The resulting mixture was heated to 120° C. overnight. After being cooled down to room temperature the reaction was quenched with water, extracted with EtOAc twice, the combined organic layers were washed with brine, dried over Na₂SO₄, filtered and concentrated. The residue was purified by flash column chromatography (silica gel, 0-50%, ethyl acetate in petroleum ether) to give crude product which was further purified by prep-HPLC to afford tert-butyl (S)-4-(7-(3-methoxyphenyl)-5-(pyridin-2-yl)-7H-pyrrolo[2,3-d]pyrimidin-4-yl)-3-methylpiperazine-1-carboxylate (13 mg, 8.0%) as a white solid. LC/MS ESI (m/z): 501 (M+H)⁺. ¹H NMR (400 MHz, DMSO-d₆) δ 8.67 (d, J=4.2 Hz, 1H), 8.42 (s, 1H), 8.04 (s, 1H), 7.94 (td, J=7.7, 1.5 Hz, 1H), 7.68 (d, J=7.8 Hz, 1H), 7.51-7.42 (m, 3H), 7.35 (dd, J=7.0, 5.1 Hz, 1H), 7.05-6.92 (m, 1H), 4.18 (d, J=5.9 Hz, 1H), 3.83 (s, 3H), 3.77-3.64 (m, 2H), 3.07-2.60 (m, 4H), 1.36 (s, 9H), 0.91 (d, J=6.5 Hz, 3H).

The following compound was prepared by an analogous procedure to the synthesis of compound 278 from the corresponding aryl halide.

| Cmpd | | |
|---|---|---|
| No. | Chemical Name | LCMS and ¹H NMR |
| 279 | tert-butyl (S)-3-methyl-4-(5- | LC/MS ESI (m/z): 555 (M + H)⁺. ¹H NMR (400 MHz, |
| | (pyridin-2-yl)-7-(3- | DMSO-d₆) δ 8.71-8.66 (m, 1H), 8.46 (s, 1H, 8.16 (s, 1H), |
| | (trifluoromethoxy)phenyl)-7H- | 8.08 (s, 1H), 8.01 (dd, J = 8.1, 1.5 Hz, 1H), 7.95 (td, J = |
| | pyrrolo[2,3-d]pyrimidin-4- | 7.7, 1.8 Hz, 1H), 7.73-7.66 (m, 2H), 7.45-7.40 (m, 1H), |
| | yl)piperazine-1-carboxylate | 7.39-7.33 (m, 1H), 4.22 (d, J = 6.2 Hz, 1H), 3.71 (s, 1H), |
| | | 3.56-3.44 (m, 2H), 3.08-2.71 (m, 3H), 1.37 (s, 9H), 0.93 |
| | | (d, J = 6.6 Hz, 3H). |

### Example 26. Synthesis of tert-butyl (S)-4-(7-(3-chlorophenyl)-5-(1H-pyrazol-1-yl)-7H-pyrrolo[2,3-d]pyrimidin-4-yl)-3-methylpiperazine-1-carboxylate (Compound 322)

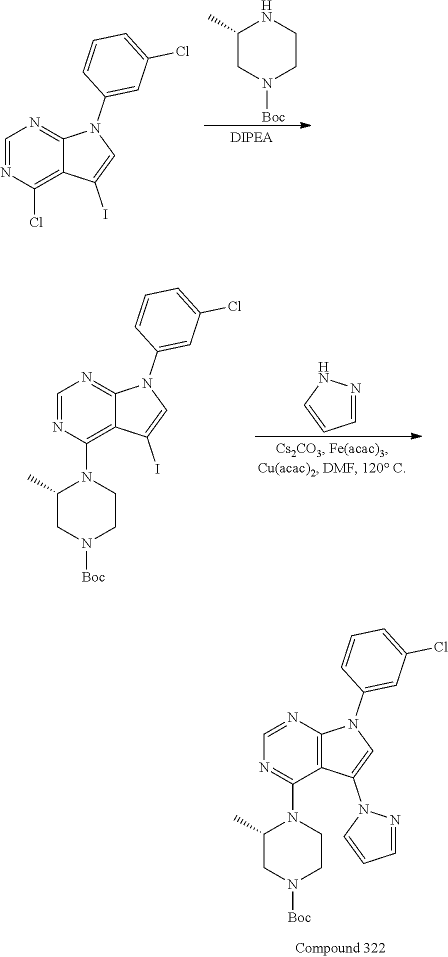

### Step 1. tert-Butyl (S)-4-(7-(3-chlorophenyl)-5-iodo-7H-pyrrolo[2,3-d]pyrimidin-4-yl)-3-methylpiperazine-1-carboxylate

To a solution of 4-chloro-7-(3-chlorophenyl)-5-iodo-7H-pyrrolo[2,3-d]pyrimidine (5.0 g, 13 mmol) in DIPEA (15 mL) was added tert-butyl (S)-3-methylpiperazine-1-carboxylate (6.4 g, 32 mmol). The resulting mixture was heated to 140° C. for 2 h. After being cooled down to room temperature, solvent was removed and the residue was purified by flash column chromatography (silica gel, 0˜30%, ethyl acetate in petroleum ether) to afford tert-butyl (S)-4-(7-(3-chlorophenyl)-5-iodo-7H-pyrrolo[2,3-d]pyrimidin-4-yl)-3-methylpiperazine-1-carboxylate (5.9 g, 83%) as a yellow solid. LC/MS ESI (m/z): 554 (M+H)⁺.

### Step 2. tert-Butyl (S)-4-(7-(3-chlorophenyl)-5-(1H-pyrazol-1-yl)-7H-pyrrolo[2,3-d]pyrimidin-4-yl)-3-methylpiperazine-1-carboxylate

To a solution of tert-butyl (S)-4-(7-(3-chlorophenyl)-5-iodo-7H-pyrrolo[2,3-d]pyrimidin-4-yl)-3-methylpiperazine-1-carboxylate (200 mg, 0.36 mmol) in DMF (6 mL) were added 1H-pyrazole (49 mg, 0.72 mmol), Cs₂CO₃ (470 mg, 1.4 mmol), Fe(acac)₃ (38 mg, 0.10 mmol) and Cu(acac)₂ (9.4 mg, 0.036 mmol). The resulting mixture was heated to 120° C. overnight. After being cooled down to room temperature, the reaction was quenched with water and extracted with EtOAc twice. The combined organic layers were washed with brine, dried over Na₂SO₄, filtered, and concentrated. The residue was purified by flash column chromatography (silica gel, 0˜40%, ethyl acetate in petroleum ether) to give crude product which was further purified by prep-HPLC to afford tert-butyl (S)-4-(7-(3-chlorophenyl)-5-(1H-pyrazol-1-yl)-7H-pyrrolo[2,3-d]pyrimidin-4-yl)-3-methylpiperazine-1-carboxylate (9.6 mg, 5.0%) as a white solid. LC/MS ESI (m/z): 494 (M+H)⁺. ¹H NMR (400 MHz, CDCl₃) δ 8.50 (s, 1H), 7.77 (d, J=1.7 Hz, 1H), 7.74 (t, J=1.8 Hz, 1H), 7.72 (d, J=2.0 Hz, 1H), 7.63-7.60 (m, 1H), 7.51 (s, 1H), 7.47 (t, J=8.0 Hz, 1H), 7.41-7.37 (m, 1H), 6.52 (t, J=2.1 Hz, 1H), 4.24-3.79 (m, 2H), 3.67 (d, J=12.8 Hz, 1H), 3.32 (m, 1H), 3.14-2.70 (m, 3H), 1.45 (s, 9H), 1.09 (s, 3H).

The following compounds were prepared by analogous procedures to the synthesis of compound 322 from the corresponding amines.

| Cmpd | | |
|---|---|---|
| No. | Chemical Name | LCMS and ¹H NMR |
| 323 | tert-butyl (S)-4-(7-(3- | LC/MS ESI (m/z): 494 (M + H)⁺. ¹H NMR (400 MHz, |
| | chlorophenyl)-5-(1H-imidazol- | CD₃OD) δ 8.42 (s, 1H), 8.00 (s, 1H), 7.94 (s, 1H), 7.92 (t, |
| | 1-yl)-7H-pyrrolo[2,3- | J = 2.0 Hz, 1H), 7.73-7.71 (m, 1H), 7.55 (t, J = 8.1 Hz, 1H), |
| | d]pyrimidin-4-yl)-3- | 7.47-7.44 (m, 2H), 7.23 (s, 1H), 4.01 (s, 1H), 3.82 (s, 1H), |
| | methylpiperazine-1-carboxylate | 3.64 (d, J = 13.2 Hz, 1H), 3.37-3.32 (m, 1H), 3.12 (td, J = |
| | | 13.0, 3.4 Hz, 1H), 3.03-2.76 (m, 2H), 1.44 (s, 9H), 1.05 |
| | | (d, J = 6.7 Hz, 3H). |
| 332 | tert-butyl (R)-4-(7-(3- | LC/MS ESI (m/z): 494 (M + H)⁺. ¹H NMR (400 MHz, |
| | chlorophenyl)-5-(1H-pyrazol-1- | CDCl₃) δ 8.48 (s, 1H), 7.78 (d, J = 1.8 Hz, 1H), 7.75-7.72 |
| | yl)-7H-pyrrolo[2,3-d]pyrimidin- | (m, 2H), 7.61 (d, J = 7.8 Hz, 1H), 7.50 (s, 1H), 7.48 (t, J = |
| | 4-yl)-2-methylpiperazine-1- | 8.0 Hz, 1H), 7.39 (dd, J = 8.1, 1.1 Hz, 1H), 6.53 (s, 1H), |
| | carboxylate | 4.19 (s, 1H), 3.67-3.53 (m, 3H), 3.13-2.89 (m, 3H), 1.45 |
| | | (s, 9H), 1.08 (d, J = 6.7 Hz, 3H). |
| 342 | tert-butyl (R)-4-(7-(3- | LC/MS ESI (m/z): 494 (M + H)⁺. ¹H NMR (400 MHz, |
| | chlorophenyl)-5-(1H-imidazol- | CDCl₃) δ 8.68 (s, 1H), 8.54 (s, 1H), 7.75 (s, 1H), 7.66- |
| | 1-yl)-7H-pyrrolo[2,3- | 7.31 (m, 6H), 4.23 (br, 1H), 3.70 (d, J = 12.3 Hz, 1H), 3.55 |
| | d]pyrimidin-4-yl)-2- | (d, J = 11.1 Hz, 1H), 3.46 (d, J = 12.7 Hz, 1H), 3.19 (d, J = |
| | methylpiperazine-1-carboxylate | 11.2 Hz, 1H), 3.09-2.88 (m, 2H), 1.44 (s, 9H), 1.04 (d, J = |
| | | 6.6 Hz, 3H). |

### Example 27. Synthesis of tert-butyl (R)-4-(7-(4-cyanopyridin-2-yl)-5-(pyridin-2-yl)-7H-pyrrolo[2,3-d]pyrimidin-4-yl)-2-methylpiperazine-1-carboxylate (Compound 367)

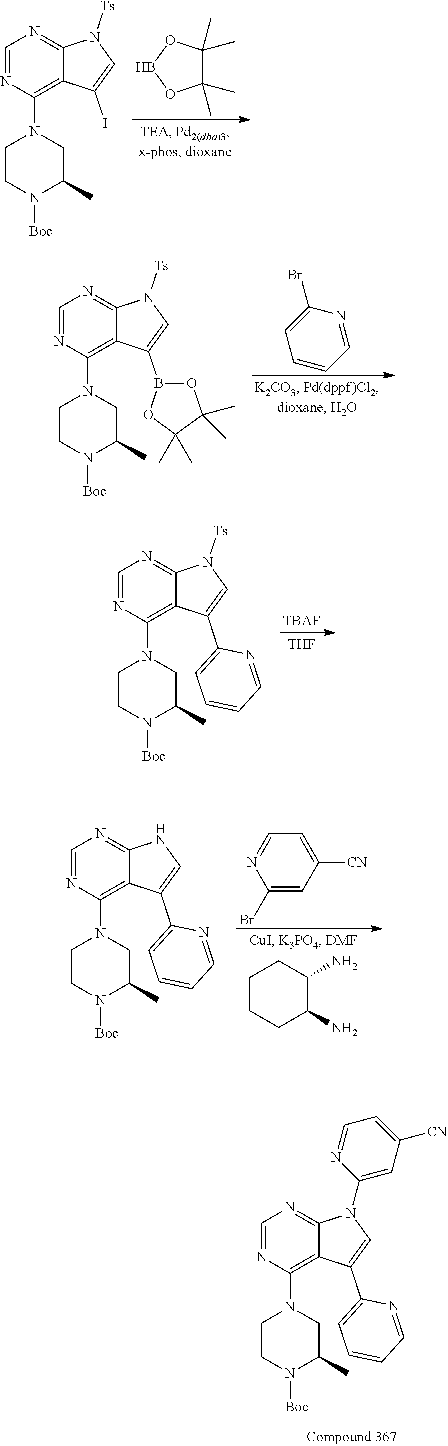

### Step 1. tert-Butyl (R)-2-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate

To a solution of tert-butyl (R)-4-(5-iodo-7-tosyl-7H-pyrrolo[2,3-d]pyrimidin-4-yl)-2-methylpiperazine-1-carboxylate (1.0 g, 1.6 mmol) in dioxane (15 mL) were added 4,4,5,5-tetramethyl-1,3,2-dioxaborolane (0.97 mL, 6.6 mmol), X-Phos (0.080 g, 0.16 mmol), TEA (1.2 mL, 8.3 mmol) and Pd₂(dba)₃ (0.15 g, 0.16 mmol). The resulting mixture was stirred at 95° C. overnight. After being cooled down to room temperature, the reaction was quenched with water and extracted with DCM twice. The combined organic layers were washed with brine, dried over Na₂SO₄, filtered, and concentrated to afford crude tert-butyl (R)-2-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate as a yellow oil, which was used in the next step directly. LC/MS ESI (m/z): 598 (M+H)⁺.

### Step 2. tert-Butyl (R)-2-methyl-4-(5-(pyridin-2-yl)-7-tosyl-7H-pyrrolo[2,3-d]pyrimidin-4-yl)piperazine-1-carboxylate

To a solution of tert-butyl (R)-2-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate (1.0 g, 1.6 mmol) in dioxane (15 mL) and H₂O (3 mL) were added 2-bromopyridine (0.32 mL, 3.3 mmol), K₂CO₃ (1.2 g, 8.3 mmol) and Pd(dppf)Cl₂ (0.12 g, 0.16 mmol). The resulting mixture was heated to 90° C. overnight. After being cooled down to room temperature, solvent was removed and the residue was purified by flash column chromatography (silica gel, 0˜50%, ethyl acetate in petroleum ether) to afford tert-butyl (R)-2-methyl-4-(5-(pyridin-2-yl)-7-tosyl-7H-pyrrolo[2,3-d]pyrimidin-4-yl)piperazine-1-carboxylate (0.72 g, 78%) as a yellow solid. LC/MS ESI (m/z): 549 (M+H)⁺.

### Step 3. tert-Butyl (R)-2-methyl-4-(5-(pyridin-2-yl)-7H-pyrrolo[2,3-d]pyrimidin-4-yl)piperazine-1-carboxylate

To a solution of tert-butyl (R)-2-methyl-4-(5-(pyridin-2-yl)-7-tosyl-7H-pyrrolo[2,3-d]pyrimidin-4-yl)piperazine-1-carboxylate (720 mg, 1.3 mmol) in THE (5 mL) was added TBAF (5.2 mL, 1.0M in THF). The resulting mixture was stirred at room temperature overnight. The reaction was quenched with water and extracted with EtOAc twice. The combined organic layers were washed with brine, dried over Na₂SO₄, filtered and concentrated. The residue was purified by flash column chromatography (silica gel, 0˜10%, methanol in dichloromethane) to afford tert-butyl (R)-2-methyl-4-(5-(pyridin-2-yl)-7H-pyrrolo[2,3-d]pyrimidin-4-yl)piperazine-1-carboxylate (510 mg, 98%) as a yellow solid. LC/MS ESI (m/z): 395 (M+H)⁺.

### Step 4. tert-Butyl (R)-4-(7-(4-cyanopyridin-2-yl)-5-(pyridin-2-yl)-7H-pyrrolo[2,3-d]pyrimidin-4-yl)-2-methylpiperazine-1-carboxylate

To a solution of tert-butyl (R)-2-methyl-4-(5-(pyridin-2-yl)-7H-pyrrolo[2,3-d]pyrimidin-4-yl)piperazine-1-carboxylate (510 mg, 1.2 mmol) in DMF (10 mL) were added 2-bromoisonicotinonitrile (470 mg, 2.5 mmol), CuI (250 mg, 1.2 mmol), trans-cyclohexane-1,2-diamine (150 mg, 1.2 mmol) and K₃PO₄ (820 mg, 3.8 mmol). The resulting mixture was heated to 120° C. overnight. After being cooled down to room temperature, the reaction was quenched with water and extracted with EtOAc twice. The combined organic layers were washed with brine, dried over Na₂SO₄, filtered, and concentrated. The residue was purified by flash column chromatography (silica gel, 0˜50%, ethyl acetate in petroleum ether) to afford tert-butyl (R)-4-(7-(4-cyanopyridin-2-yl)-5-(pyridin-2-yl)-7H-pyrrolo[2,3-d]pyrimidin-4-yl)-2-methylpiperazine-1-carboxylate (600 mg, 93%) as a yellow solid. 100 mg of the product was further purified by prep-HPLC to obtain 14.8 mg of the tert-butyl (R)-4-(7-(4-cyanopyridin-2-yl)-5-(pyridin-2-yl)-7H-pyrrolo[2,3-d]pyrimidin-4-yl)-2-methylpiperazine-1-carboxylate as a white solid. LC/MS ESI (m/z): 497 (M+H)⁺. ¹H NMR (400 MHz, CDCl₃) δ 9.40 (s, 1H), 8.73 (d, J=4.3 Hz, 1H), 8.63 (dd, J=5.0, 0.5 Hz, 1H), 8.59-8.53 (m, 2H), 7.85-7.79 (m, 1H), 7.64 (d, J=7.8 Hz, 1H), 7.41 (dd, J=5.0, 1.3 Hz, 1H), 7.34-7.29 (m, 1H), 4.24-4.19 (m, 1H), 3.84 (d, J=12.9 Hz, 2H), 3.58-3.53 (m, 1H), 3.08 (dd, J=13.2, 3.9 Hz, 1H), 2.89-2.83 (m, 2H), 1.43 (s, 9H), 1.09 (d, J=6.8 Hz, 3H).

### Example 28. Synthesis of tert-butyl (S)-4-(7-(3-cyanophenyl)-5-(pyrrolidin-1-ylmethyl)-7H-pyrrolo[2,3-d]pyrimidin-4-yl)-3-methylpiperazine-1-carboxylate (Compound 400)

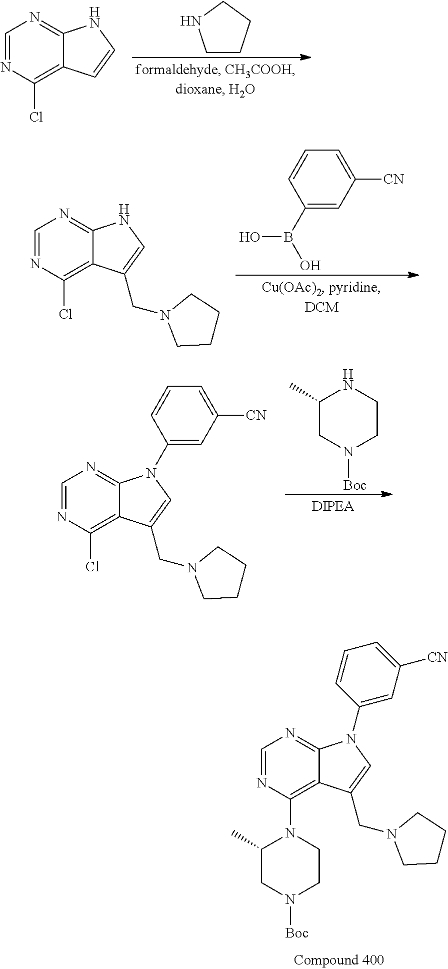

### Step 1. 4-chloro-5-(pyrrolidin-1-ylmethyl)-7H-pyrrolo[2,3-d]pyrimidine

To a 0° C. solution of formaldehyde (0.58 g, 7.1 mmol) in H₂O (2 mL) and CH₃COOH (10 mL, 86 mmol) and dioxane (10 mL). was added pyrrolidine (0.58 mL, 7.1 mmol), followed by a solution of 4-chloro-7H-pyrrolo[2,3-d]pyrimidine (1.0 g, 6.5 mmol) in dioxane dropwise. After being stirred at 50° C. overnight under N₂, the reaction was added to anhydrous sodium sulfate to dry and then filtered. The filtrate was concentrated at room temperature and purified by flash column chromatography (silica gel, 0˜50%, methanol in dichloromethane) to afford 4-chloro-5-(pyrrolidin-1-ylmethyl)-7H-pyrrolo[2,3-d]pyrimidine (0.63 g, 40%) as a yellow oil. LC/MS ESI (m/z): 237 (M+H)⁺.

### Step 2. 3-(4-chloro-5-(pyrrolidin-1-ylmethyl)-7H-pyrrolo[2,3-d]pyrimidin-7-yl) benzonitrile

To a solution of 4-chloro-5-(pyrrolidin-1-ylmethyl)-7H-pyrrolo[2,3-d]pyrimidine (0.53 g, 2.2 mmol) in DCM (25 mL) were added (3-cyanophenyl)boronic acid (0.66 g, 4.4 mmol), Cu(OAc)₂ (1.2 g, 6.7 mmol), pyridine (1.1 mL, 13 mmol) and 4 A molecular sieves (800 mg). The resulting mixture was stirred at room temperature under O₂ atmosphere overnight. The reaction was quenched with NH₄OH (2 mL) at 0° C. and filtered. The filtrate was extracted with DCM twice and the combined organic layers were dried over Na₂SO₄, filtered and concentrated. The residue was purified by flash column chromatography (silica gel, 0˜20%, methanol in dichloromethane) to afford 3-(4-chloro-5-(pyrrolidin-1-ylmethyl)-7H-pyrrolo[2,3-d]pyrimidin-7-yl)benzonitrile (0.26 g, 34%) as a yellow solid. LC/MS ESI (m/z): 338 (M+H)⁺.

### Step 3. tert-butyl (S)-4-(7-(3-cyanophenyl)-5-(pyrrolidin-1-ylmethyl)-7H-pyrrolo[2,3-d]pyrimidin-4-yl)-3-methylpiperazine-1-carboxylate

To a solution of 3-(4-chloro-5-(pyrrolidin-1-ylmethyl)-7H-pyrrolo[2,3-d]pyrimidin-7-yl)benzonitrile (100 mg, 0.29 mmol) in DIPEA (1 mL) was added tert-butyl (S)-3-methylpiperazine-1-carboxylate (240 mg, 1.1 mmol). The resulting mixture was heated to 140° C. for 3 h. After being cooled down to room temperature, the solvent was removed and the residue was purified by flash column chromatography (silica gel, 0˜20%, methanol in dichloromethane). The product was further purified by prep-HPLC to afford tert-butyl (S)-4-(7-(3-cyanophenyl)-5-(pyrrolidin-1-ylmethyl)-7H-pyrrolo[2,3-d]pyrimidin-4-yl)-3-methylpiperazine-1-carboxylate (75 mg, 50%) as a white solid. LC/MS ESI (m/z): 502 (M+H)⁺. ¹H NMR (400 MHz, MeOD) δ 8.38 (s, 1H), 8.24 (s, 1H), 8.13-8.03 (m, 1H), 7.77-7.69 (m, 2H), 7.65 (s, 1H), 4.52 (s, 1H), 4.06-3.91 (m, 2H), 3.90-3.65 (m, 3H), 3.58-3.40 (m, 2H), 3.27-3.10 (m, 1H), 2.71-2.58 (m, 4H), 1.86-1.74 (m, 4H), 1.50 (s, 9H), 1.17 (d, J=6.4 Hz, 3H).

### Example 29. Synthesis of tert-butyl (S)-4-(7-(3-cyanophenyl)-5-(1-methylcyclopropyl)-7H-pyrrolo[2,3-d]pyrimidin-4-yl)-3-methylpiperazine-1-carboxylate (Compound 402)

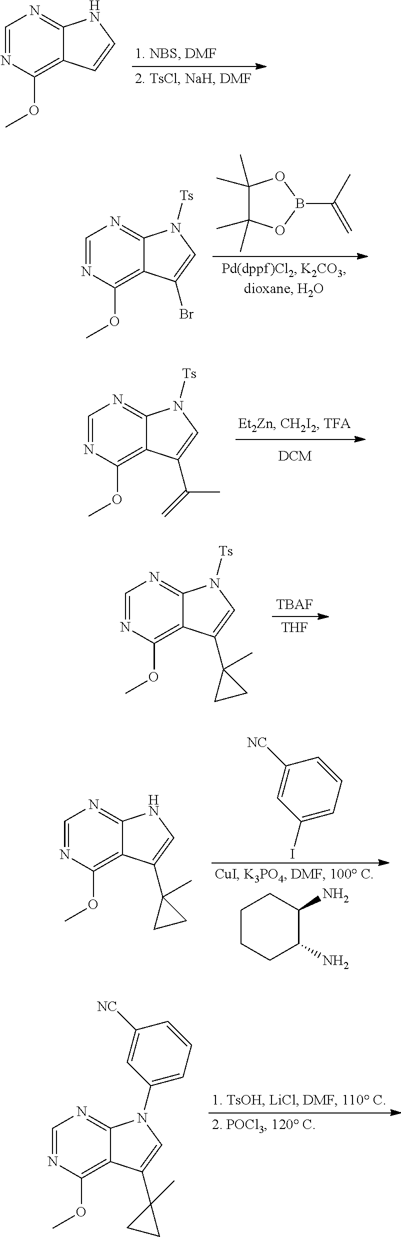

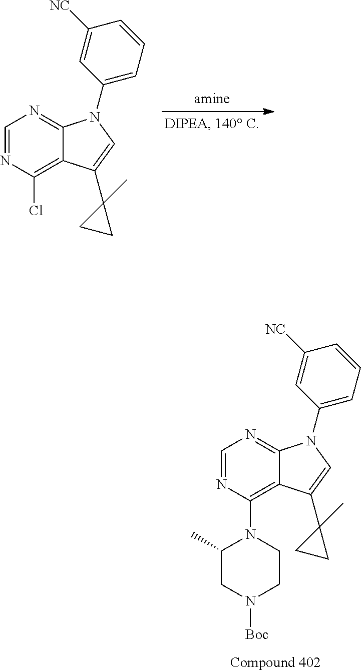 [SMILES: CC1(c2cn(-c3cccc(C#N)c3)c3ncnc(Cl)c23)CC1 ; C[C@H]1CN(C(=O)OC(C)(C)C)CCN1c1ncnc2c1c(C1(C)CC1)cn2-c1cccc(C#N)c1]

### Step 1. 5-Bromo-4-methoxy-7H-pyrrolo[2,3-d]pyrimidine

At 0° C., to a solution of 4-methoxy-7H-pyrrolo[2,3-d]pyrimidine (1.0 g, 6.7 mmol) in DMF (15 mL) was added NBS (0.95 g, 5.3 mmol) in portions. After being stirred at room temperature for 3 h under N₂, the reaction was quenched with water. The reaction mixture was filtered to afford 5-bromo-4-methoxy-7H-pyrrolo[2,3-d]pyrimidine (0.45 g, 29%) as a yellow solid. LC/MS ESI (m/z): 228, 230 (M+H)⁺.

### Step 2. 5-Bromo-4-methoxy-7-tosyl-7H-pyrrolo[2,3-d]pyrimidine

At 0° C., to a solution of 5-bromo-4-methoxy-7H-pyrrolo[2,3-d]pyrimidine (450 mg, 1.9 mmol) in DMF (10 mg) was added NaH (95 mg, 2.3 mmol) in portions. The resulting mixture was stirred at 0° C. for 20 min. Then to above mixture was added 4-methylbenzenesulfonyl chloride (430 mg, 2.2 mmol) and the resulting mixture was stirred at 0° C. for 20 min and then was allowed to warm up to room temperature and stirred overnight under N₂. The reaction was quenched with ice water. Then the reaction mixture was filtered to afford 5-bromo-4-methoxy-7-tosyl-7H-pyrrolo[2,3-d]pyrimidine (690 mg, 91%) as a gray solid. LC/MS ESI (m/z): 382,384 (M+H)⁺.

### Step 3. 4-Methoxy-5-(prop-1-en-2-yl)-7-tosyl-7H-pyrrolo[2,3-d]pyrimidine

To a solution of 5-bromo-4-methoxy-7-tosyl-7H-pyrrolo[2,3-d]pyrimidine (690 mg, 1.8 mmol) in dioxane (10 mL) and H₂O (2 mL) were added 4,4,5,5-tetramethyl-2-(prop-1-en-2-yl)-1,3,2-dioxaborolane (0.51 mL, 2.7 mmol), K₂CO₃ (1000 mg, 7.2 mmol) and Pd(dppf)Cl₂ (130 mg, 0.18 mmol). The resulting mixture was heated to 90° C. overnight. After being cooled down to room temperature, solvent was removed and the residue was purified by flash column chromatography (silica gel, 0˜30%, ethyl acetate in petroleum ether) to afford 4-methoxy-5-(prop-1-en-2-yl)-7-tosyl-7H-pyrrolo[2,3-d]pyrimidine (460 mg, 74%) as a white solid. LC/MS ESI (m/z): 344 (M+H)⁺.

### Step 4. 4-Methoxy-5-(I-methylcyclopropyl)-7-tosyl-7H-pyrrolo[2,3-d]pyrimidine

To a solution of Et₂Zn (13 mL) in DCM (10 mL) at 0° C. was added dropwise a solution of TFA (0.50 mL, 6.6 mmol) in DCM (2 mL) and the mixture was stirred at 0° C. for 30 min. Then to the above mixture was added dropwise a solution of CH₂I₂ (0.56 mL, 6.6 mmol) in DCM (2 mL). After being stirred for 20 min, a solution of 4-methoxy-5-(prop-1-en-2-yl)-7-tosyl-7H-pyrrolo[2,3-d]pyrimidine (460 mg, 1.3 mmol) in DCM (5 mL) was added dropwise. The resulting mixture was stirred at room temperature overnight. The reaction was quenched with NH₄Cl (aq.), extracted with DCM twice, the combined organic layers were washed with brine, dried over Na₂SO₄, filtered and concentrated. The residue was purified by flash column chromatography (silica gel, 0˜10%, ethyl acetate in petroleum ether) to afford 4-methoxy-5-(1-methylcyclopropyl)-7-tosyl-7H-pyrrolo[2,3-d]pyrimidine (350 mg, 73%) as a yellow oil. LC/MS ESI (m/z): 358 (M+H)⁺.

### Step 5. 4-Methoxy-5-(I-methylcyclopropyl)-7H-pyrrolo[2,3-d]pyrimidine

To a solution of 4-methoxy-5-(1-methylcyclopropyl)-7-tosyl-7H-pyrrolo[2,3-d]pyrimidine (350 mg, 0.98 mmol) in THE (3 mL) was added TBAF (3.9 mL, 1.0M in THF). The resulting mixture was stirred at 30° C. for 4 h. The reaction was quenched with water, extracted with EtOAc twice, the combined organic layers were washed with brine, dried over Na₂SO₄, filtered and concentrated. The residue was purified by flash column chromatography (silica gel, 0˜60%, ethyl acetate in petroleum ether) to give crude product which was further purified by prep-HPLC to afford 4-methoxy-5-(1-methylcyclopropyl)-7H-pyrrolo[2,3-d]pyrimidine (40 mg, 20%) as a white solid. LC/MS ESI (m/z): 204 (M+H)⁺.

### Step 6. 3-(4-Methoxy-5-(I-methylcyclopropyl)-7H-pyrrolo[2,3-d]pyrimidin-7-yl) benzonitrile

To a solution of 4-methoxy-5-(1-methylcyclopropyl)-7H-pyrrolo[2,3-d]pyrimidine (40 mg, 0.19 mmol) in DMF (5 mL) were added 3-iodobenzonitrile (180 mg, 0.78 mmol), CuI (37 mg, 0.19 mmol), trans-cyclohexane-1,2-diamine (45 mg, 0.39 mmol) and K₃PO₄ (130 mg, 0.59 mmol). The resulting mixture was heated to 100° C. overnight. After being cooled down to room temperature, the reaction was quenched with water, extracted with EtOAc twice, the combined organic layers were washed with brine, dried over Na₂SO₄, filtered, and concentrated. The residue was purified by flash column chromatography (silica gel, 0˜30%, ethyl acetate in petroleum ether) to afford 3-(4-methoxy-5-(1-methylcyclopropyl)-7H-pyrrolo[2,3-d]pyrimidin-7-yl) benzonitrile (55 mg, 91%) as a yellow solid. LC/MS ESI (m/z): 305 (M+H)⁺.

### Step 7. 3-(4-Hydroxy-5-(I-methylcyclopropyl)-7H-pyrrolo[2,3-d]pyrimidin-7-yl) benzonitrile

To a solution of 3-(4-methoxy-5-(1-methylcyclopropyl)-7H-pyrrolo[2,3-d]pyrimidin-7-yl)benzonitrile (55 mg, 0.18 mmol) in DMF (2 mL) were added p-toluenesulfonic acid (310 mg, 1.8 mmol) and LiCl (77 mg, 1.8 mmol). The resulting mixture was heated to 110° C. for 2 h. After being cooled down to room temperature, the reaction was quenched with water and extracted with EtOAc twice. The combined organic layers were washed with brine, dried over Na₂SO₄, filtered, and concentrated to afford 3-(4-hydroxy-5-(1-methylcyclopropyl)-7H-pyrrolo[2,3-d]pyrimidin-7-yl) benzonitrile (50 mg, 95%) as a yellow solid. LC/MS ESI (m/z): 291 (M+H)⁺.

### Step 8. 3-(4-Chloro-5-(1-methylcyclopropyl)-7H-pyrrolo[2,3-d]pyrimidin-7-yl) benzonitrile

The mixture of 3-(4-hydroxy-5-(1-methylcyclopropyl)-7H-pyrrolo[2,3-d]pyrimidin-7-yl)benzonitrile (50 mg, 0.17 mmol) and POCl₃ (5 mL) was heated to 120° C. overnight. After being cooled down to room temperature, the reaction was concentrated. The residue was dissolved in DCM, washed with NaHCO₃ (aq.), and the aqueous layer was extracted with DCM twice. The combined organic layers were washed with brine, dried over Na₂SO₄, filtered, and concentrated to afford 3-(4-chloro-5-(1-methylcyclopropyl)-7H-pyrrolo[2,3-d]pyrimidin-7-yl)benzonitrile (50 mg, 94%) as a yellow solid. LC/MS ESI (m/z): 309 (M+H)⁺.

### Step 9. tert-Butyl (S)-4-(7-(3-cyanophenyl)-5-(I-methylcyclopropyl)-7H-pyrrolo[2,3-d]pyrimidin-4-yl)-3-methylpiperazine-1-carboxylate

To a solution of 3-(4-chloro-5-(1-methylcyclopropyl)-7H-pyrrolo[2,3-d]pyrimidin-7-yl)benzonitrile (50 mg, 0.16 mmol) in DIPEA (1 mL) was added tert-butyl (S)-3-methylpiperazine-1-carboxylate (320 mg, 1.6 mmol). The resulting mixture was heated to 140° C. for 6 h. After being cooled down to room temperature, the solvent was removed and the residue was purified by flash column chromatography (silica gel, 0˜20%, ethyl acetate in petroleum ether) to give the product. Further purification by prep-HPLC provided tert-butyl (S)-4-(7-(3-cyanophenyl)-5-(1-methylcyclopropyl)-7H-pyrrolo[2,3-d]pyrimidin-4-yl)-3-methylpiperazine-1-carboxylate (4.3 mg, 5.0%) as a white solid. LC/MS ESI (m/z): 473 (M+H)⁺. ¹H NMR (400 MHz, CD₃OD) δ 8.38-8.30 (m, 1H), 8.27-8.18 (m, 1H), 8.08-8.01 (m, 1H), 7.73-7.66 (m, 2H), 7.47-7.42 (m, 1H), 4.97-4.86 (m, 1H), 4.07 (d, J=12.9 Hz, 1H), 4.02-3.89 (m, 1H), 3.86 (d, J=12.6 Hz, 1H), 3.61-3.36 (m, 2H), 3.25-3.05 (m, 1H), 1.54 (s, 3H), 1.50 (d, J=5.9 Hz, 9H), 1.34-1.28 (m, 2H), 1.16 (d, J=6.5 Hz, 3H), 0.95-0.88 (m, 1H), 0.83-0.71 (m, 1H).

The following compound was prepared by the procedure analogous to the synthesis of compound 402 from the corresponding aryl halide.

| Cmpd | | |
|---|---|---|
| No. | Chemical Name | LCMS and ¹H NMR |
| 411 | tert-butyl (S)-4-(7-(4- | LC/MS ESI (m/z): 474 (M + H)⁺. ¹H NMR (400 MHz, |
| | cyanopyridin-2-yl)-5-(1- | CDCl₃) δ 9.32 (s, 1H), 8.60 (d, J = 5.0 Hz, 1H), 8.50 (s, |
| | methylcyclopropyl)-7H- | 1H), 7.94 (s, 1H), 7.35 (dd, J = 5.0, 1.1 Hz, 1H), 4.86 (s, |
| | pyrrolo[2,3-d]pyrimidin-4-yl)-3- | 1H), 4.17-3.80 (m, 3H), 3.59-3.40 (m, 2H), 3.21-2.99 |
| | methylpiperazine-1-carboxylate | (m, 1H), 1.51 (s, 12H), 1.28-1.22 (m, 1H), 1.19 (d, J = 5.1 |
| | | Hz, 3H), 0.92-0.83 (m, 2H), 0.81-0.74 (m, 1H). |

### Example 30. Synthesis of ethyl 4-(5-cyclopropyl-7-(5-methoxypyridin-3-yl)-7H-pyrrolo[2,3-d]pyrimidin-4-yl)piperazine-1-carboxylate (Compound 1004)

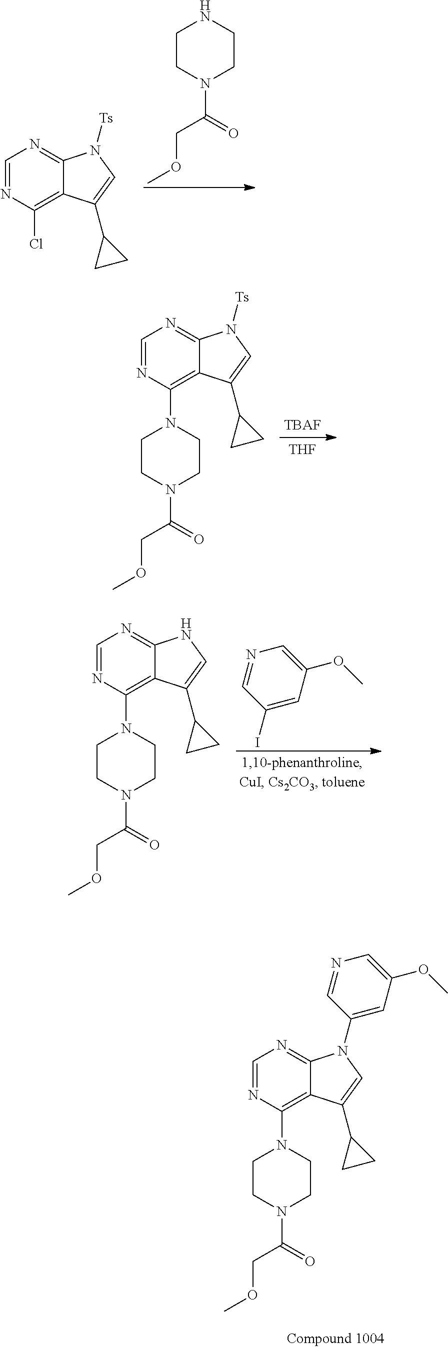

### Step 1. Ethyl 4-(5-cyclopropyl-7-tosyl-7H-pyrrolo[2,3-d]pyrimidin-4-yl)piperazine-1-carboxylate

A mixture of 4-chloro-5-cyclopropyl-7-tosyl-7H-pyrrolo[2,3-d]pyrimidine (400 mg, 1.2 mmol) and ethyl piperazine-1-carboxylate (220 mg, 1.4 mmol) was heated to 100° C. overnight. After cooling down to room temperature, the reaction mixture was purified by flash column chromatography (silica gel, 0˜30%, ethyl acetate in petroleum ether) to afford ethyl 4-(5-cyclopropyl-7-tosyl-7H-pyrrolo[2,3-d]pyrimidin-4-yl)piperazine-1-carboxylate (360 mg, 67%) as a white solid. LC/MS ESI (m/z): 470 (M+H)⁺.

### Step 2. Ethyl 4-(5-cyclopropyl-7H-pyrrolo[2,3-d]pyrimidin-4-yl)piperazine-1-carboxylate

To a solution of ethyl 4-(5-cyclopropyl-7-tosyl-7H-pyrrolo[2,3-d]pyrimidin-4-yl)piperazine-1-carboxylate (360 mg, 0.77 mmol) in THE (5 mL) was added TBAF (5.3 mL, 1.0M in THF). The resulting mixture was stirred at room temperature overnight. The reaction was quenched with water and extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to afford ethyl 4-(5-cyclopropyl-7H-pyrrolo[2,3-d]pyrimidin-4-yl)piperazine-1-carboxylate (200 mg, 83%) as a yellow solid. LC/MS ESI (m/z): 316 (M+H)⁺.

### Step 3. Ethyl 4-(5-cyclopropyl-7-(5-methoxypyridin-3-yl)-7H-pyrrolo[2,3-d]pyrimidin-4-yl)piperazine-1-carboxylate

To a solution of ethyl 4-(5-cyclopropyl-7H-pyrrolo[2,3-d]pyrimidin-4-yl)piperazine-1-carboxylate (100 mg, 0.32 mmol) in toluene (15 mL) were added 3-iodo-5-methoxypyridine (82 mg, 0.35 mmol), 1,10-phenanthroline (57 mg, 0.32 mmol), CuI (60 mg, 0.32 mmol) and Cs₂CO₃ (310 mg, 0.95 mmol). The resulting mixture was heated to 110° C. overnight. After cooling down to room temperature, the reaction was quenched with water, and extracted twice with EtOAc. The combined organic layers were washed with brine, dried over Na₂SO₄, filtered and concentrated. The residue was purified by flash column chromatography (silica gel, 0˜20%, ethyl acetate in petroleum ether) to give crude product which was further purified by prep-HPLC to afford ethyl 4-(5-cyclopropyl-7-(5-methoxypyridin-3-yl)-7H-pyrrolo[2,3-d]pyrimidin-4-yl)piperazine-1-carboxylate (31 mg, 23%) as a white solid. LC/MS ESI (m/z): 423 (M+H)⁺. ¹H NMR (400 MHz, DMSO-d6) δ 8.71 (d, J=2.1 Hz, 1H), 8.38 (s, 1H), 8.28 (d, J=2.6 Hz, 1H), 7.88 (t, J=2.4 Hz, 1H), 7.59 (s, 1H), 4.09 (q, J=7.1 Hz, 2H), 3.91 (s, 3H), 3.66-3.62 (m, 4H), 3.62-3.57 (m, 4H), 2.09-2.01 (m, 1H), 1.22 (t, J=7.1 Hz, 3H), 1.03-0.97 (m, 2H), 0.88-0.83 (m, 2H).

The following compounds were prepared by procedures analogous to the synthesis of compound 1004 using the corresponding amine.

| Cmpd | | |
|---|---|---|
| No. | Chemical Name | LCMS and ¹H NMR |
| 104 | tert-butyl 4-(5-cyclopropyl-7-(5- | LC/MS ESI (m/z): 451 (M + H)⁺. ¹H NMR (400 MHz, |
| | methoxypyridin-3-yl)-7H- | DMSO-d6) δ 8.71 (d, J = 2.1 Hz, 1H), 8.38 (s, 1H), 8.28 (d, |
| | pyrrolo[2,3-d]pyrimidin-4- | J = 2.6 Hz, 1H), 7.88 (t, J = 2.4 Hz, 1H), 7.59 (s, 1H), 3.91 |
| | yl)piperazine-1-carboxylate | (s, 3H), 3.63-3.59 (m, 4H), 3.56-3.52 (m, 4H), 2.09- |
| | | 2.01 (m, 1H), 1.44 (s, 9H), 1.02-0.97 (m, 2H), 0.87-0.83 |
| | | (m, 2H). |

### Example 31. Synthesis of ethyl (S)-4-(7-(4-chlorophenyl)-5-cyclopropyl-7H-pyrrolo[2,3-d]pyrimidin-4-yl)-3-methylpiperazine-1-carboxylate (Compound 112)

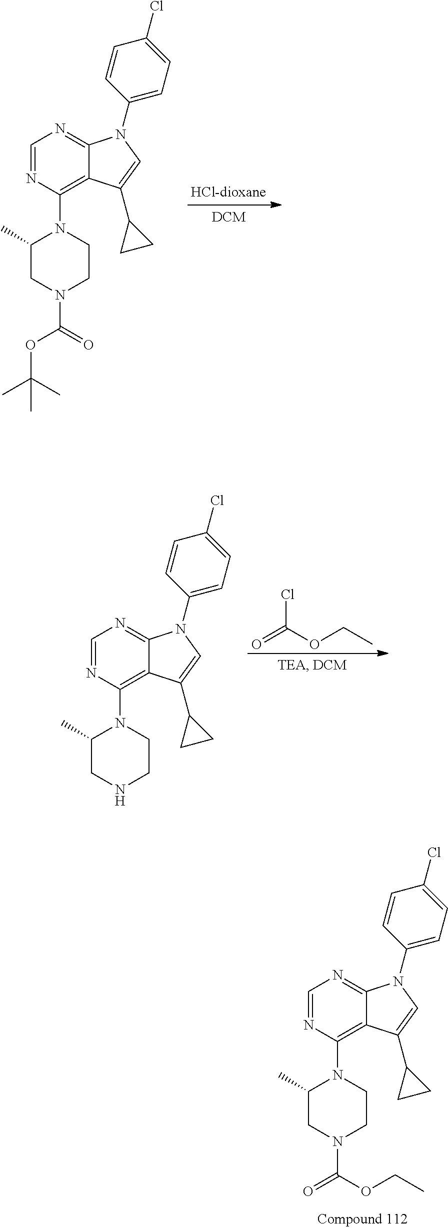

### Step 1 (S)-7-(4-Chlorophenyl)-5-cyclopropyl-4-(2-methylpiperazin-1-yl)-7H-pyrrolo[2,3-d]pyrimidine

To a solution of tert-butyl (S)-4-(7-(4-chlorophenyl)-5-cyclopropyl-7H-pyrrolo[2,3-d]pyrimidin-4-yl)-3-methylpiperazine-1-carboxylate (500 mg, 1.1 mmol, prepared following the procedures described for the synthesis of compound 114 in DCM (10 mL) was added HCl (3.0 mL, 4.0M in dioxane). The resulting mixture was stirred at room temperature for 2 h. After removal of solvent, the residue was diluted with DCM and washed with NaHCO₃ (aq.). The aqueous layer was extracted twice with DCM. The combined organic layers were washed with brine, dried over Na₂SO₄, filtered and concentrated. The residue was used in the next step directly. (S)-7-(4-chlorophenyl)-5-cyclopropyl-4-(2-methylpiperazin-1-yl)-7H-pyrrolo[2,3-d]pyrimidine (350 mg, 89% yield). LC/MS ESI (m/z): 368 (M+H)⁺.

### Step 2 Ethyl (S)-4-(7-(4-chlorophenyl)-5-cyclopropyl-7H-pyrrolo[2,3-d]pyrimidin-4-yl)-3-methylpiperazine-1-carboxylate

To a solution of (S)-7-(4-chlorophenyl)-5-cyclopropyl-4-(2-methylpiperazin-1-yl)-7H-pyrrolo[2,3-d]pyrimidine (90 mg, 0.25 mmol) in DCM (5 mL) at 0° C. was added TEA (76 mg, 0.75 mmol), followed by ethyl carbonochloridate (54 mg, 0.50 mmol) dropwise. The resulting mixture was stirred at room temperature for 2 h. The reaction was quenched with water and extracted twice with DCM. The combined organic layers were washed with NaHCO₃ (aq.), dried over Na₂SO₄, filtered and concentrated. The residue was purified by flash column chromatography (silica gel, 0-30%, ethyl acetate in petroleum ether) to give crude product which was further purified by prep-HPLC to afford ethyl (S)-4-(7-(4-chlorophenyl)-5-cyclopropyl-7H-pyrrolo[2,3-d]pyrimidin-4-yl)-3-methylpiperazine-1-carboxylate (34 mg, 31% yield) as a white solid. LC/MS ESI (m/z): 440 (M+H)⁺. ¹H NMR (400 MHz, CDCl₃) δ 8.43 (s, 1H), 7.62-7.58 (m, 2H), 7.48-7.44 (m, 2H), 6.90 (d, J=0.7 Hz, 1H), 4.81-4.70 (m, 1H), 4.24-4.17 (m, 2H), 4.16-4.01 (m, 1H), 4.00-3.81 (m, 2H), 3.65-3.51 (m, 1H), 3.44-3.30 (m, 1H), 3.28-3.13 (m, 1H), 2.04 (td, J=8.0, 4.1 Hz, 1H), 1.30 (t, J=7.1 Hz, 3H), 1.25 (d, J=6.6 Hz, 3H), 1.04-0.99 (m, 2H), 0.83-0.77 (m, 1H), 0.74-0.68 (m, 1H).

### Example 32. Synthesis of tert-butyl (S)-4-(7-(4-chlorophenyl)-5-cyclopropyl-7H-pyrrolo[2,3-d]pyrimidin-4-yl)-3-methylpiperazine-1-carboxylate (Compound 114)

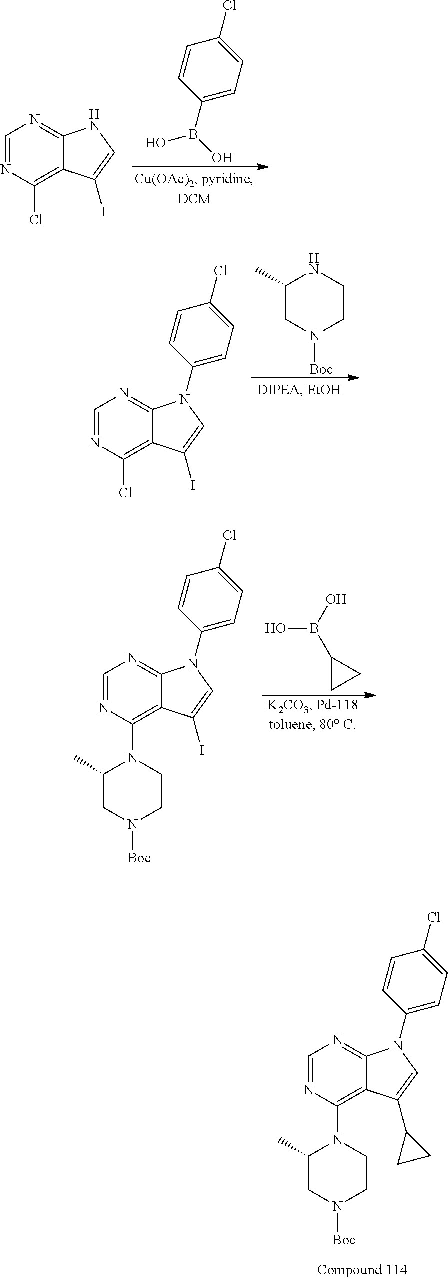

### Step 1. 4-Chloro-7-(4-chlorophenyl)-5-iodo-7H-pyrrolo[2,3-d]pyrimidine

To a solution of 4-chloro-5-iodo-7H-pyrrolo[2,3-d]pyrimidine (1.3 g, 4.7 mmol) in DCM (50 mL) were added (4-chlorophenyl)boronic acid (1.5 g, 9.3 mmol), Cu(OAc)₂ (2.1 g, 12 mmol) and pyridine (2.2 mL, 28 mmol). The resulting mixture was stirred at room temperature overnight. NH₃—H₂O (30 mL) was added and the reaction was filtered. The filtrate was partitioned between DCM and water. The aqueous phase was extracted twice with DCM. The combined organic layers were washed with brine, dried over Na₂SO₄, filtered and concentrated. The residue was purified by flash chromatography (silica gel, 0-30% EtOAc in petroleum ether) to afford 4-chloro-7-(4-chlorophenyl)-5-iodo-7H-pyrrolo[2,3-d]pyrimidine (810 mg, 45%) as a solid. LC/MS ESI (m/z): 390 (M+H)⁺.

### Step 2. tert-Butyl (S)-4-(7-(4-chlorophenyl)-5-iodo-7H-pyrrolo[2,3-d]pyrimidin-4-yl)-3-methylpiperazine-1-carboxylate

To a solution of 4-chloro-7-(4-chlorophenyl)-5-iodo-7H-pyrrolo[2,3-d]pyrimidine (800 mg, 2.1 mmol) in DIPEA (5 mL) was added tert-butyl (S)-3-methylpiperazine-1-carboxylate (820 mg, 4.1 mmol). The resulting mixture was heated to 140° C. for 3 h. After cooling to room temperature, solvent was removed and the residue was purified by flash column chromatography (silica gel, 0˜30%, ethyl acetate in petroleum ether) to afford tert-butyl (S)-4-(7-(4-chlorophenyl)-5-iodo-7H-pyrrolo[2,3-d]pyrimidin-4-yl)-3-methylpiperazine-1-carboxylate (900 mg, 79%) as a white solid. LC/MS ESI (m/z): 554 (M+H)⁺.

### Step 3. tert-Butyl (S)-4-(7-(4-chlorophenyl)-5-cyclopropyl-7H-pyrrolo[2,3-d]pyrimidin-4-yl)-3-methylpiperazine-1-carboxylate

To a solution of tert-butyl (S)-4-(7-(4-chlorophenyl)-5-iodo-7H-pyrrolo[2,3-d]pyrimidin-4-yl)-3-methylpiperazine-1-carboxylate (900 mg, 1.6 mmol) in toluene (15 mL) were added cyclopropylboronic acid (280 mg, 0.16 mmol), K₂CO₃ (2.9 g, 21 mmol) and Pd-118 (100 mg, 1.3 mmol). The resulting mixture was heated to 80° C. overnight. After cooling to room temperature, the reaction was filtered. The filtrate was concentrated and purified by flash column chromatography (silica gel, 0˜30%, ethyl acetate in petroleum ether) to give tert-butyl (S)-4-(7-(4-chlorophenyl)-5-cyclopropyl-7H-pyrrolo[2,3-d]pyrimidin-4-yl)-3-methylpiperazine-1-carboxylate (580 mg, 76% yield) as a solid. LC/MS ESI (m/z): 468 (M+H)⁺. ¹H NMR (400 MHz, CDCl₃) δ 8.43 (s, 1H), 7.62-7.58 (m, 2H), 7.48-7.44 (m, 2H), 6.89 (s, 1H), 4.78-4.69 (m, 1H), 4.15-3.80 (m, 3H), 3.60-3.50 (m, 1H), 3.40-3.27 (m, 1H), 3.22-3.05 (m, 1H), 2.07-2.00 (m, 1H), 1.50 (s, 9H), 1.24 (d, J=6.6 Hz, 3H), 1.04-0.99 (m, 2H), 0.82-0.76 (m, 1H), 0.73-0.67 (m, 1H).

The following compounds were prepared by procedures analogous to the synthesis of compound 114 using the corresponding boronic acids and amine.

| Cmpd | | |
|---|---|---|
| No. | Chemical Name | LCMS and ¹H NMR |
| 139 | tert-butyl (S)-4-(7-(3- | LC/MS ESI (m/z): 468 (M + H)⁺. ¹H NMR (400 MHz, |
| | chlorophenyl)-5-cyclopropyl- | CDCl₃) δ8.44 (s, 1H), 7.68 (t, J = 2.0 Hz, 1H), 7.62-7.59 |
| | 7H-pyrrolo[2,3-d]pyrimidin-4- | (m, 1H), 7.42 (t, J = 8.1 Hz, 1H), 7.32-7.28 (m, 1H), 6.91 |
| | yl)-3-methylpiperazine-1- | (s, 1H), 4.78-4.69 (m, 1H), 4.15-3.82 (m, 3H), 3.60- |
| | carboxylate | 3.50 (m, 1H), 3.40-3.28 (m, 1H), 3.21-3.04 (m, 1H), |
| | | 2.07-2.01 (m, 1H), 1.50 (s, 9H), 1.24 (d, J = 6.6 Hz, 3H), |
| | | 1.04-1.00 (m, 2H), 0.82-0.76 (m, 1H), 0.74-0.67 (m, |
| | | 1H). |
| 147 | tert-butyl (R)-4-(7-(4- | LC/MS ESI (m/z): 468 (M + H)⁺. ¹H NMR (400 MHz, |
| | chlorophenyl)-5-cyclopropyl- | CDCl₃) δ8.43 (s, 1H), 7.62-7.58 (m, 2H), 7.48-7.44 (m, |
| | 7H-pyrrolo[2,3-d]pyrimidin-4- | 2H), 6.89 (d, J = 0.7 Hz, 1H), 4.81-4.68 (m, 1H), 4.15- |
| | yl)-3-methylpiperazine-1- | 3.79 (m, 3H), 3.62-3.50 (m, 1H), 3.40-3.27 (m, 1H), |
| | carboxylate | 3.22-3.04 (m, 1H), 2.08-2.01 (m, 1H), 1.50 (s, 9H), 1.24 |
| | | (d, J = 6.5 Hz, 3H), 1.02 (dd, J = 8.2, 1.9 Hz, 2H), 0.83- |
| | | 0.76 (m, 1H), 0.73-0.67 (m, 1H). |

### Example 33. Synthesis of tert-butyl (S)-4-(7-(3-chlorophenyl)-5-(pyridin-2-yl)-7H-pyrrolo[2,3-d]pyrimidin-4-yl)-3-methylpiperazine-1-carboxylate (Compound 119)

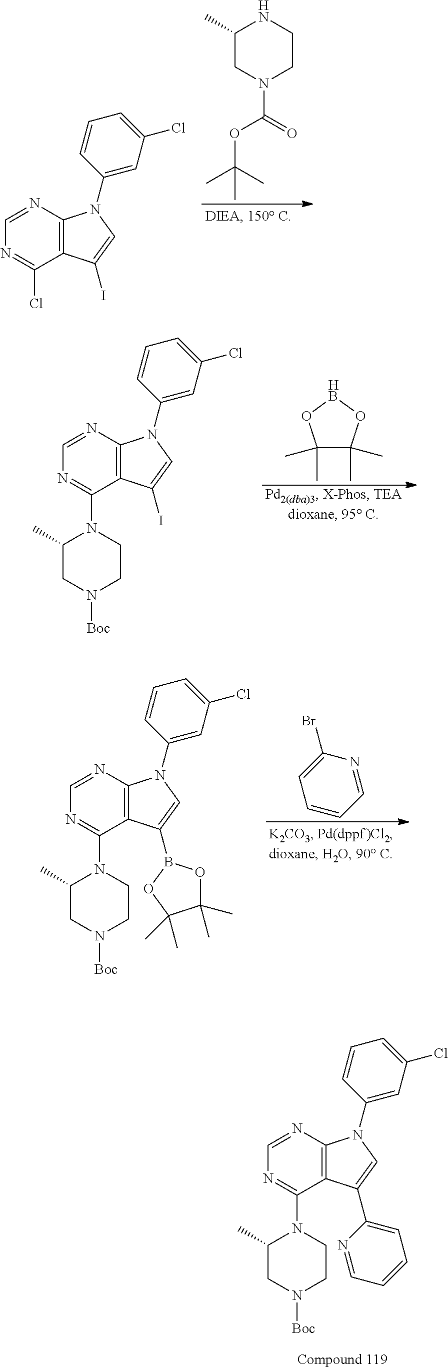

### Step 1. tert-Butyl (S)-4-(7-(3-chlorophenyl)-5-iodo-7H-pyrrolo[2,3-d]pyrimidin-4-yl)-3-methylpiperazine-1-carboxylate

A mixture of 4-chloro-7-(3-chlorophenyl)-5-iodo-7H-pyrrolo[2,3-d]pyrimidine (3.0 g, 7.7 mmol, prepared following step 1 of the procedures described for compound 274), tert-butyl (S)-3-methylpiperazine-1-carboxylate (3.1 g, 15 mmol) in DIEA (20 mL) was heated at 150° C. for 6 hours. The reaction mixture was concentrated. The residue was purified by column chromatography on silica gel (0˜30% ethyl acetate in petroleum ether) to give tert-butyl (S)-4-(7-(3-chlorophenyl)-5-iodo-7H-pyrrolo[2,3-d]pyrimidin-4-yl)-3-methylpiperazine-1-carboxylate (3.5 g, 6.2 mmol, 81%) as a yellow solid. LC/MS ESI (m/z): 554 (M+H)⁺.

### Step 2. tert-Butyl (S)-4-(7-(3-chlorophenyl)-5-(4,4,5,5-tetramethyl-1,3,2-dioxaborolan-2-yl)-7H-pyrrolo[2,3-d]pyrimidin-4-yl)-3-methylpiperazine-1-carboxylate

A mixture of tert-butyl (S)-4-(7-(3-chlorophenyl)-5-iodo-7H-pyrrolo[2,3-d]pyrimidin-4-yl)-3-methylpiperazine-1-carboxylate (3.5 g, 6.2 mmol), 4,4,5,5-tetramethyl-1,3,2-dioxaborolane (3.6 mL, 25 mmol), Pd₂(dba)₃ (0.60 g, 0.62 mmol), X-Phos (0.60 g, 1.3 mmol) and TEA (4.3 mL, 31 mmol) in dioxane (60 mL) was heated at 95° C. for 12 hours. The reaction mixture was filtered. The filtrate was extracted with EtOAc (100 mL×2). The combined organic layers were washed with brine, dried over Na₂SO₄ and concentrated. The residue was used in the next step directly. LC/MS ESI (m/z): 554 (M+H)⁺.

### Step 3. tert-Butyl (S)-4-(7-(3-chlorophenyl)-5-(pyridin-2-yl)-7H-pyrrolo[2,3-d]pyrimidin-4-yl)-3-methylpiperazine-1-carboxylate

A mixture of tert-butyl (S)-4-(7-(3-chlorophenyl)-5-(4,4,5,5-tetramethyl-1,3,2-dioxaborolan-2-yl)-7H-pyrrolo[2,3-d]pyrimidin-4-yl)-3-methylpiperazine-1-carboxylate (1.0 g, 1.8 mmol), 2-bromopyridine (0.35 mL, 3.6 mmol), Pd(dppf)Cl₂ (130 mg, 0.18 mmol) and K₂CO₃ (1.3 g, 9.0 mmol) in dioxane (20 mL) and H₂O (2 mL) was stirred at 90° C. for 12 hours. The reaction mixture was concentrated. The residue was purified by column chromatography on silica gel (0˜50% ethyl acetate in petroleum ether) to give the crude product. The crude product was purified by prep-HPLC to give the tert-butyl (S)-4-(7-(3-chlorophenyl)-5-(pyridin-2-yl)-7H-pyrrolo[2,3-d]pyrimidin-4-yl)-3-methylpiperazine-1-carboxylate (300 mg, 33%) as a white solid. LC/MS ESI (m/z): 505 (M+H)⁺. ¹H NMR (400 MHz, CDCl3) δ 8.70 (d, J=4.2 Hz, 1H), 8.53 (s, 1H), 7.82-7.76 (m, 2H), 7.69 (d, J=7.7 Hz, 2H), 7.61 (d, J=7.9 Hz, 1H), 7.47 (t, J=8.1 Hz, 1H), 7.36 (d, J=8.1 Hz, 1H), 7.29-7.24 (m, 1H), 4.40-4.16 (m, 1H), 4.03-3.76 (m, 1H), 3.58 (t, J=15.4 Hz, 2H), 3.17 (t, J=11.8 Hz, 1H), 3.06-2.71 (m, 2H), 1.44 (s, 9H), 1.04 (br. s, 3H).

The following compounds were prepared by procedures analogous to the synthesis of compound 119 using the corresponding aryl halides.

| Cmpd | | |
|---|---|---|
| No. | Chemical Name | LCMS and ¹H NMR |
| 120 | tert-butyl (S)-4-(7-(3- | LC/MS ESI (m/z): 506 (M + H)⁺. ¹H NMR (400 MHz, |
| | chlorophenyl)-5-(pyrazin-2-yl)- | CDCl₃) δ 8.94 (s, 1H), 8.63 (dd, J = 2.4, 1.6 Hz, 1H), 8.57 |
| | 7H-pyrrolo[2,3-d]pyrimidin-4- | (s, 1H), 8.53 (d, J = 2.5 Hz, 1H), 7.82-7.79 (m, 2H), 7.71- |
| | yl)-3-methylpiperazine-1- | 7.67 (m, 1H), 7.49 (t, J = 8.1 Hz, 1H), 7.39 (ddd, J = 8.1, |
| | carboxylate | 1.9, 1.0 Hz, 1H), 4.21 (d, J = 43.8 Hz, 1H), 3.98-3.78 (m, |
| | | 1H), 3.67-3.47 (m, 2H), 3.23 (t, J = 11.7 Hz, 1H), 2.94 |
| | | (dd, J = 72.0, 43.1 Hz, 2H), 1.44 (s, 9H), 1.08 (br. s, 3H). |
| 115 | tert-butyl (S)-4-(7-(3- | LC/MS ESI (m/z): 506 (M + H)⁺. ¹H NMR (400 MHz, |
| | chlorophenyl)-5-(pyrimidin-4- | CDCl₃) δ 9.23 (d, J = 1.2 Hz, 1H), 8.78 (d, J = 5.3 Hz, 1H), |
| | yl)-7H-pyrrolo[2,3-d]pyrimidin- | 8.56 (s, 1H), 7.98 (s, 1H), 7.80 (t, J = 2.0 Hz, 1H), 7.68 (d, |
| | 4-yl)-3-methylpiperazine-1- | J = 6.0 Hz, 2H), 7.49 (t, J = 8.0 Hz, 1H), 7.43-7.37 (m, |
| | carboxylate | 1H), 4.32 (br. s, 1H), 4.11-3.81 (m, 1H), 3.78-3.50 (m, |
| | | 2H), 3.28 (t, J = 11.2 Hz, 1H), 3.20-2.74 (m, 2H), 1.45 (s, |
| | | 9H), 1.10 (d, J = 6.6 Hz, 3H). |
| 116 | tert-butyl (S)-4-(7-(3- | LC/MS ESI (m/z): 506 (M + H)⁺. ¹H NMR (400 MHz, |
| | chlorophenyl)-5-(pyrimidin-2- | CD₃OD) δ 8.92 (d, J = 4.9 Hz, 2H), 8.39 (s, 1H), 8.12 (s, |
| | yl)-7H-pyrrolo[2,3-d]pyrimidin- | 1H), 7.93 (t, J = 2.0 Hz, 1H), 7.73 (dd, J = 8.0, 1.0 Hz, 1H), |
| | 4-yl)-3-methylpiperazine-1- | 7.58 (t, J = 8.1 Hz, 1H), 7.48 (dd, J = 8.1, 0.9 Hz, 1H), 7.42 |
| | carboxylate | (t, J = 4.9 Hz, 1H), 4.47 (br. s, 1H), 3.90 (d, J = 13.2 Hz, |
| | | 1H), 3.71 (t, J = 14.5 Hz, 2H), 3.20 (td, J = 13.0, 3.3 Hz, |
| | | 1H), 3.14-2.76 (m, 2H), 1.46 (s, 9H), 1.10 (d, J = 6.0 Hz, |
| | | 3H). |
| 327 | tert-butyl (S)-4-(7-(3- | LC/MS ESI (m/z): 506.5 (M + H)⁺. ¹H NMR (400 MHz, |
| | chlorophenyl)-5-(pyrimidin-5- | CD₃OD) δ 9.16 (s, 1H), 9.08 (d, J = 2.1 Hz, 2H), 8.53- |
| | yl)-7H-pyrrolo[2,3-d]pyrimidin- | 8.48 (m, 1H), 8.01 (d, J = 2.5 Hz, 1H), 7.96 (d, J = 1.9 Hz, |
| | 4-yl)-3-methylpiperazine-1- | 1H), 7.77 (d, J = 8.1 Hz, 1H), 7.56 (td, J = 8.1, 2.5 Hz, 1H), |
| | carboxylate | 7.46 (d, J = 8.1 Hz, 1H), 4.05 (br. s, 1H), 3.80 (d, J = 12.3 |
| | | Hz, 1H), 3.56 (d, J = 13.1 Hz, 1H), 3.45 (d, J = 12.8 Hz, |
| | | 1H), 3.20 (t, J = 12.1 Hz, 1H), 2.92 (m, 2H), 1.43 (s, 9H), |
| | | 1.01 (d, J = 6.6 Hz, 3H). |
| 117 | tert-butyl (2R,5S)-4-(7-(3- | LC/MS ESI (m/z): 519 (M + H)⁺. ¹H NMR(400 MHz, |
| | chlorophenyl)-5-(pyridin-2-yl)- | CDCl3) δ 8.69 (d, J = 4.9 Hz, 1H), 8.53 (s, 1H), 7.84-7.81 |
| | 7H-pyrrolo[2,3-d]pyrimidin-4- | (m, 1H), 7.80-7.76 (m, 1H), 7.72-7.69 (m, 2H), 7.61 (d, |
| | yl)-2,5-dimethylpiperazine-1- | J = 7.8 Hz, 1H), 7.49-7.45 (m, 1H), 7.38-7.35 (m, 1H), |
| | carboxylate | 7.28-7.25 (m, 1H), 4.35-4.13 (m, 2H), 3.45-3.30 (m, |
| | | 3H), 3.21-3.03 (m, 1H), 1.44 (s, 9H), 1.10 (d, J = 6.7 Hz, |
| | | 3H), 1.01 (d, J = 6.6 Hz, 3H). |
| 124 | tert-butyl (2R,5S)-4-(7-(3- | LC/MS ESI (m/z): 520 (M + H)⁺. ¹H NMR(400 MHz, |
| | chlorophenyl)-5-(pyrazin-2-yl)- | CDCl3) δ 8.86 (d, J = 1.3 Hz, 1H), 8.57-8.55 (m, 1H), |
| | 7H-pyrrolo[2,3-d]pyrimidin-4- | 8.50 (s, 1H), 8.46 (d, J = 2.5 Hz, 1H), 7.76-7.74 (m, 1H), |
| | yl)-2,5-dimethylpiperazine-1- | 7.73 (s, 1H), 7.64-7.61 (m, 1H), 7.44-7.40 (m, 1H), 7.34- |
| | carboxylate | 7.31 (m, 1H), 4.31-4.09 (m, 2H), 3.39-3.29 (m, 3H), |
| | | 3.20-2.96 (m, 1H), 1.37 (s, 9H), 1.06 (d, J = 6.7 Hz, 3H), |
| | | 0.98 (d, J = 6.3 Hz, 3H). |

### Example 34. Synthesis of 1,1,1-trifluoro-2-methylpropan-2-yl (S)-4-(7-(3-chlorophenyl)-5-(pyridin-2-yl)-7H-pyrrolo[2,3-d]pyrimidin-4-yl)-3-methylpiperazine-1-carboxylate (Compound 121)

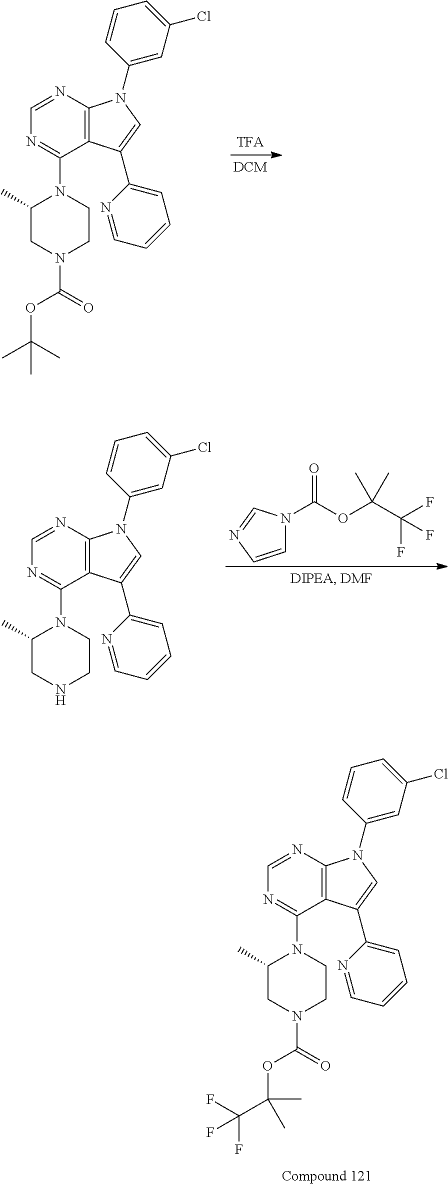 [SMILES: C[C@H]1CN(C(=O)OC(C)(C)C)CCN1c1ncnc2c1c(-c1ccccn1)cn2-c1cccc(Cl)c1 ; C[C@H]1CNCCN1c1ncnc2c1c(-c1ccccn1)cn2-c1cccc(Cl)c1 ; CC(C)(OC(=O)n1ccnc1)C(F)(F)F ; C[C@H]1CN(C(=O)OC(C)(C)C(F)(F)F)CCN1c1ncnc2c1c(-c1ccccn1)cn2-c1cccc(Cl)c1]

### Step 1. (S)-7-(3-Chlorophenyl)-4-(2-methylpiperazin-1-yl)-5-(pyridin-2-yl)-7H-pyrrolo[2,3-d]pyrimidine

To a solution of tert-butyl (S)-4-(7-(3-chlorophenyl)-5-(pyridin-2-yl)-7H-pyrrolo[2,3-d]pyrimidin-4-yl)-3-methylpiperazine-1-carboxylate (200 mg, 0.40 mmol, prepared following the procedures described for compound 119 in DCM (10 mL) was added TFA (5.0 mL, 67 mmol). The resulting mixture was stirred at room temperature for 2 hours. The reaction mixture was basified with NaHCO₃ (aq.) to pH 8 and then extracted with DCM (100 mL×2). The combined organic layers were washed with brine, dried over Na₂SO₄ and concentrated to afford (S)-7-(3-chlorophenyl)-4-(2-methylpiperazin-1-yl)-5-(pyridin-2-yl)-7H-pyrrolo[2,3-d]pyrimidine (170 mg, 94%) as a yellow solid which was used in the next step directly without further purification. LC/MS ESI (m/z): 405 (M+H)⁺.

### Step 2. 1,1,1-Trifluoro-2-methylpropan-2-yl (S)-4-(7-(3-chlorophenyl)-5-(pyridin-2-yl)-7H-pyrrolo[2,3-d]pyrimidin-4-yl)-3-methylpiperazine-1-carboxylate

A mixture of (S)-7-(3-chlorophenyl)-4-(2-methylpiperazin-1-yl)-5-(pyridin-2-yl)-7H-pyrrolo[2,3-d]pyrimidine (170 mg, 0.41 mmol), 1,1,1-trifluoro-2-methylpropan-2-yl 1H-imidazole-1-carboxylate (110 mg, 0.50 mmol) and DIEA (0.20 mL, 1.2 mmol) in DMF (10 mL) was stirred at 80° C. for 24 hours. After cooling to room temperature, the reaction mixture was concentrated. The residue was purified by column chromatography on silica gel (0˜50% ethyl acetate in petroleum ether) to give the crude product. The crude product was purified by prep-HPLC to give the 1,1,1-trifluoro-2-methylpropan-2-yl (S)-4-(7-(3-chlorophenyl)-5-(pyridin-2-yl)-7H-pyrrolo[2,3-d]pyrimidin-4-yl)-3-methylpiperazine-1-carboxylate (54 mg, 24%) as a white solid. LC/MS ESI (m/z): 559 (M+H)⁺. ¹H NMR (400 MHz, CD₃OD) δ 8.70-8.68 (m, 1H), 8.45 (s, 1H), 8.01 (td, J=7.8, 1.7 Hz, 1H), 7.96 (t, J=1.9 Hz, 1H), 7.90 (s, 1H), 7.78-7.73 (m, 2H), 7.58 (t, J=8.1 Hz, 1H), 7.49-7.43 (m, 2H), 4.26 (d, J=6.4 Hz, 1H), 3.82 (dd, J=44.3, 12.4 Hz, 1H), 3.58 (dd, J=32.0, 12.9 Hz, 2H), 3.17 (td, J=13.1, 3.2 Hz, 1H), 3.07-2.81 (m, 2H), 1.67 (s, 6H), 1.02 (d, J=6.6 Hz, 3H).

### Example 35. Synthesis of tert-butyl (R)-4-(7-(3-chloro-5-fluorophenyl)-5-cyclopropyl-7H-pyrrolo[2,3-d]pyrimidin-4-yl)-2-methylpiperazine-1-carboxylate (Compound 123)

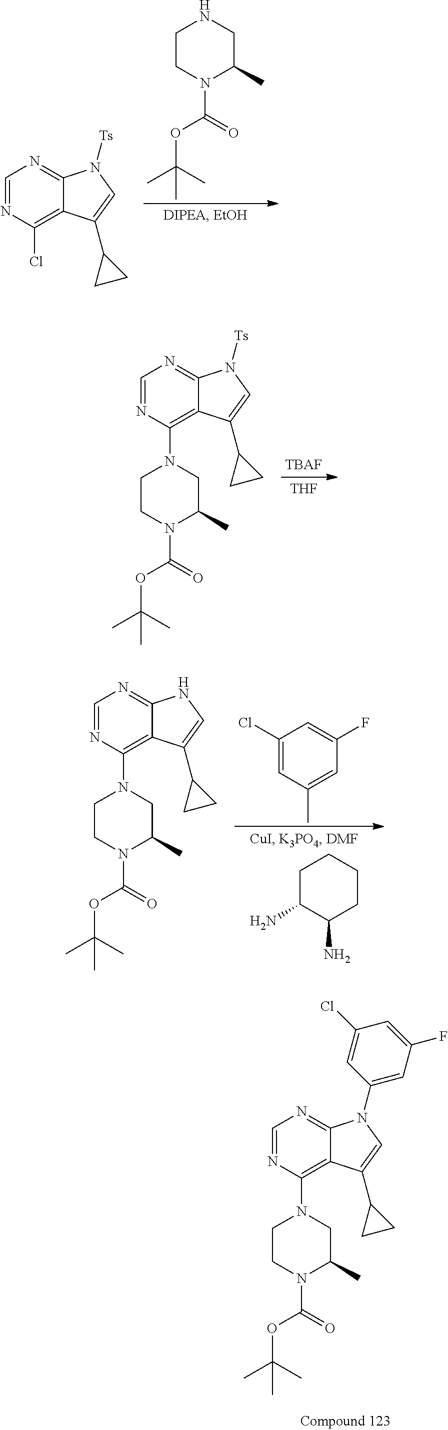

### Step 1. tert-Butyl (R)-4-(5-cyclopropyl-7-tosyl-7H-pyrrolo[2,3-d]pyrimidin-4-yl)-2-methylpiperazine-1-carboxylate

To a solution of 4-chloro-5-cyclopropyl-7-tosyl-7H-pyrrolo[2,3-d]pyrimidine (300 mg, 0.87 mmol, prepared following step 2 of the procedures described for compound 164 in EtOH (5 mL) were added tert-butyl (R)-2-methylpiperazine-1-carboxylate (350 mg, 1.7 mmol) and DIPEA (670 mg, 5.2 mmol). The resulting mixture was heated to 100° C. overnight. After cooling to room temperature, the solvent was removed and the residue was purified by flash column chromatography (silica gel, 0˜30%, ethyl acetate in petroleum ether) to afford tert-butyl (R)-4-(5-cyclopropyl-7-tosyl-7H-pyrrolo[2,3-d]pyrimidin-4-yl)-2-methylpiperazine-1-carboxylate (300 mg, 73%) as a white solid. LC/MS ESI (m/z): 512 (M+H)⁺.

### Step 2. tert-Butyl (R)-4-(5-cyclopropyl-7H-pyrrolo[2,3-d]pyrimidin-4-yl)-2-methylpiperazine-1-carboxylate

To a solution of tert-butyl (R)-4-(5-cyclopropyl-7-tosyl-7H-pyrrolo[2,3-d]pyrimidin-4-yl)-2-methylpiperazine-1-carboxylate (300 mg, 0.60 mmol) in THE (5 mL) was added TBAF (3.6 mL, 1.0M in THF). The resulting mixture was stirred at room temperature overnight. The reaction was quenched with water and extracted twice with EtOAc. The combined organic layers were washed with brine, dried over Na₂SO₄, filtered and concentrated. The residue was purified by flash column chromatography (silica gel, 0˜60%, ethyl acetate in petroleum ether) to afford tert-butyl (R)-4-(5-cyclopropyl-7H-pyrrolo[2,3-d]pyrimidin-4-yl)-2-methylpiperazine-1-carboxylate (190 mg, 88%) as a white solid. LC/MS ESI (m/z): 358 (M+H)⁺.

### Step 3. tert-Butyl (R)-4-(7-(3-chloro-5-fluorophenyl)-5-cyclopropyl-7H-pyrrolo[2,3-d]pyrimidin-4-yl)-2-methylpiperazine-1-carboxylate

To a solution of tert-butyl (3S)-1-(5-cyclopropyl-7H-pyrrolo[2,3-d]pyrimidin-4-yl)-3-methylpiperidine-4-carboxylate (80 mg, 0.23 mmol) in DMF (5 mL) were added 1-chloro-3-fluoro-5-iodobenzene (120 mg, 0.46 mmol), trans-cyclohexane-1,2-diamine (7.9 mg, 0.070 mmol), CuI (13 mg, 0.070 mmol) and K₃PO₄ (150 mg, 0.69 mmol). The resulting mixture was heated to 100° C. overnight. After cooling to room temperature, the reaction was quenched with water, extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to give crude product which was further purified by prep-HPLC to afford tert-butyl (R)-4-(7-(3-chloro-5-fluorophenyl)-5-cyclopropyl-7H-pyrrolo[2,3-d]pyrimidin-4-yl)-2-methylpiperazine-1-carboxylate (61 mg, 56%) as a solid. LC/MS ESI (m/z): 486 (M+H)⁺. ¹H NMR (400 MHz, CD₃OD) δ 8.34 (s, 1H), 7.77 (s, 1H), 7.69-7.62 (m, 1H), 7.33 (s, 1H), 7.23-7.18 (m, 1H), 4.51 (d, J=12.7 Hz, 1H), 4.43 (s, 1H), 4.13 (d, J=13.1 Hz, 1H), 3.96 (d, J=13.3 Hz, 1H), 3.49-3.36 (m, 2H), 3.15-3.06 (m, 1H), 2.13-2.02 (m, 1H), 1.51 (s, 9H), 1.23 (d, J=6.8 Hz, 3H), 1.10-1.04 (m, 2H), 1.02-0.96 (m, 1H), 0.76-0.69 (m, 1H).

The following compound was prepared by procedures analogous to the synthesis of compound 123 using the corresponding amines and aryl halides.

| Cmpd | | |
|---|---|---|
| No. | Chemical Name | LCMS and ¹H NMR |
| 166 | tert-butyl 4-(5-cyclopropyl-7-(5- | LC/MS ESI (m/z): 449 (M + H)⁺. ¹H NMR (400 MHz, |
| | ethylpyridin-3-yl)-7H- | CDCl3) δ 8.68 (d, J = 2.4 Hz, 1H), 8.45-8.42 (m, 2H), |
| | pyrrolo[2,3-d]pyrimidin-4- | 7.92 (t, J = 2.1 Hz, 1H), 6.94 (d, J = 0.7 Hz, 1H), 3.71 (s, |
| | yl)piperazine-1-carboxylate | 4H), 3.65-3.61 (m, 4H), 2.77 (q, J = 7.6 Hz, 2H), 2.10- |
| | | 2.03 (m, 1H), 1.50 (s, 9H), 1.34-1.30 (m, 3H), 1.06-1.01 |
| | | (m, 2H), 0.80-0.76 (m, 2H) |
| 384 | tert-butyl (R)-4-(7-(4- | LC/MS ESI (m/z): 460 (M + H)⁺. ¹H NMR (400 MHz, |
| | cyanopyridin-2-yl)-5- | CDCl₃) δ 9.33 (s, 1H), 8.59-8.57 (m, 1H), 8.50 (s, 1H), |
| | cyclopropyl-7H-pyrrolo[2,3- | 7.80 (d, J = 0.6 Hz, 1H), 7.35-7.33 (m, 1H), 4.48-4.39 |
| | d]pyrimidin-4-yl)-2- | (m, 2H), 4.09 (d, J = 12.9 Hz, 1H), 3.97 (d, J = 13.2 Hz, |
| | methylpiperazine-1-carboxylate | 1H), 3.42-3.31 (m, 2H), 3.12-3.04 (m, 1H), 2.05-1.99 |
| | | (m, 1H), 1.50 (s, 9H), 1.22 (d, J = 6.8 Hz, 3H), 1.07-1.02 |
| | | (m, 2H), 0.99-0.93 (m, 1H), 0.74-0.68 (m, 1H). |
| 108 | tert-butyl (R)-4-(5-cyclopropyl- | LC/MS ESI (m/z): 470 (M + H)⁺. ¹H NMR (400 MHz, |
| | 7-(3,5-difluorophenyl)-7H- | CDCl₃) δ8.45 (s, 1H), 7.41-7.31 (m, 2H), 6.93 (d, J = |
| | pyrrolo[2,3-d]pyrimidin-4-yl)- | 0.7 Hz, 1H), 6.81-6.71 (m, 1H), 4.53-4.35 (m, 2H), 4.09 |
| | 2-methylpiperazine-1- | (d, J = 13.0 Hz, 1H), 3.97 (d, J = 13.2 Hz, 1H), 3.46-3.26 |
| | carboxylate | (m, 2H), 3.09 (td, J = 12.4, 3.4 Hz, 1H), 2.10-1.97 (m, |
| | | 1H), 1.50 (s, 9H), 1.22 (d, J = 6.8 Hz, 3H), 1.04 (dt, J = 8.2, |
| | | 4.0 Hz, 2H), 0.95-0.81 (m, 1H), 0.74-0.61 (m, 1H). |
| 110 | tert-butyl (R)-4-(7-(4-chloro-3- | LC/MS ESI (m/z): 486 (M + H)⁺. ¹H NMR (400 MHz, |
| | fluorophenyl)-5-cyclopropyl- | CDCl₃) δ8.43 (s, 1H), 7.62 (dd, J = 10.1, 2.3 Hz, 1H), 7.54- |
| | 7H-pyrrolo[2,3-d]pyrimidin-4- | 7.41 (m, 2H), 6.92 (d, J = 0.8 Hz, 1H), 4.53-4.33 (m, 2H), |
| | yl)-2-methylpiperazine-1- | 4.10 (d, J = 12.9 Hz, 1H), 3.96 (d, J = 13.2 Hz, 1H), 3.43- |
| | carboxylate | 3.28 (m, 2H), 3.08 (td, J = 12.4, 3.3 Hz, 1H), 2.08-1.98 (m, |
| | | 1H), 1.50 (s, 9H), 1.22 (d, J = 6.8 Hz, 3H), 1.08-0.99 (m, |
| | | 2H), 0.91-0.82 (m, 1H), 0.71-0.62 (m, 1H). |
| 111 | tert-butyl (R)-4-(5-cyclopropyl- | LC/MS ESI (m/z): 502 (M + H)⁺. ¹H NMR (400 MHz, |
| | 7-(3,5-dichlorophenyl)-7H- | CDCl₃) δ8.44 (s, 1H), 7.67 (d, J = 1.7 Hz, 2H), 7.30 (t, J = |
| | pyrrolo[2,3-d]pyrimidin-4-yl)- | 1.7 Hz, 1H), 6.91 (s, 1H), 4.53-4.36 (m, 2H), 4.10 (d, J = |
| | 2-methylpiperazine-1- | 12.9 Hz, 1H), 3.96 (d, J = 13.4 Hz, 1H), 3.45-3.29 (m, 2H), |
| | carboxylate | 3.08 (td, J = 12.4, 3.3 Hz, 1H), 2.09-1.97 (m, 1H), 1.50 (s, |
| | | 9H), 1.22 (d, J = 6.7 Hz, 3H), 1.07-0.99 (m, 2H), 0.87 (dd, |
| | | J = 9.3, 4.6 Hz, 1H), 0.71-0.60 (m, 1H). |
| 109 | tert-butyl (R)-4-(5-cyclopropyl- | LC/MS ESI (m/z): 470 (M + H)⁺. ¹H NMR (400 MHz, |
| | 7-(3,4-difluorophenyl)-7H- | CDCl₃) δ8.43 (s, 1H), 7.64-7.53 (m, 1H), 7.38 (d, J = |
| | pyrrolo[2,3-d]pyrimidin-4-yl)- | 9.0 Hz, 1H), 7.32-7.21 (m, 1H), 6.89 (s, 1H), 4.54-4.37 |
| | 2-methylpiperazine-1- | (m, 2H), 4.10 (d, J = 12.9 Hz, 1H), 3.97 (d, J = 13.4 Hz, 1H), |
| | carboxylate | 3.48-3.30 (m, 2H), 3.09 (td, J = 12.3, 3.3 Hz, 1H), 2.10- |
| | | 1.97 (m, 1H), 1.50 (s, 9H), 1.22 (d, J = 6.8 Hz, 3H), 1.10- |
| | | 0.98 (m, 2H), 0.91-0.75 (m, 1H), 0.80-0.57 (m, 1H). |
| 107 | tert-butyl 4-(7-(4-chlorophenyl)- | LC/MS ESI (m/z): 454 (M + H)⁺. ¹H NMR (400 MHz, |
| | 5-cyclopropyl-7H-pyrrolo[2,3- | CDCl₃) δ 8.44 (s, 1H), 7.60 (d, J = 8.6 Hz, 2H), 7.46 (d, J = |
| | d]pyrimidin-4-yl) piperazine-1- | 8.6 Hz, 2H), 6.90 (s, 1H), 3.73-3.67 (m, 4H), 3.64-3.60 |
| | carboxylate | (m, 4H), 2.09-2.02 (m, 1H), 1.50 (s, 9H), 1.05-1.00 (m, |
| | | 2H), 0.78-0.74 (m, 2H). |
| 113 | tert-butyl 4-(5-cyclopropyl-7- | LC/MS ESI (m/z): 456 (M + H)⁺. ¹H NMR (400 MHz, |
| | (3,4-difluorophenyl)-7H- | CDCl₃) δ 8.44 (s, 1H), 7.63-7.55 (m, 1H), 7.41-7.35 (m, |
| | pyrrolo[2,3-d]pyrimidin-4-yl) | 1H), 7.30 (d, J = 8.7 Hz, 1H), 6.88 (d, J = 0.6 Hz, 1H), 3.72- |
| | piperazine-1-carboxylate | 3.60 (m, 8H), 2.09-2.02 (m, 1H), 1.50 (s, 9H), 1.05- |
| | | 1.00 (m, 2H), 0.78-0.74 (m, 2H). |
| 122 | tert-butyl 4-(5-cyclopropyl-7- | LC/MS ESI (m/z): 488 (M + H)⁺. ¹H NMR (400 MHz, |
| | (3,5-dichlorophenyl)-7H- | CDCl₃) δ 8.46 (s, 1H), 7.67 (d, J = 1.8 Hz, 2H), 7.31 (t, J = |
| | pyrrolo[2,3-d]pyrimidin-4-yl) | 1.8 Hz, 1H), 6.90 (s, 1H), 3.69 (s, 4H), 3.63-3.59 (m, 4H), |
| | piperazine-1-carboxylate | 2.09-2.01 (m, 1H), 1.50 (s, 9H), 1.06-1.01 (m, 2H), 0.79- |
| | | 0.74 (m, 2H). |
| 205 | tert-butyl (R)-4-(5-cyclopropyl- | LC/MS ESI (m/z): 470 (M + H)⁺. ¹H NMR (400 MHz, |
| | 7-(2,3-difluorophenyl)-7H- | CDCl3) δ 8.42 (s, 1H), 7.41-7.36 (m, 1H), 7.24-7.19 (m, |
| | pyrrolo[2,3-d]pyrimidin-4-yl)-2- | 2H), 6.89-6.84 (m, 1H), 4.51 (d, J = 10.3 Hz, 1H), 4.46- |
| | methylpiperazine-1-carboxylate | 4.38 (m, 1H), 4.11 (d, J = 12.9 Hz, 1H), 3.97 (d, J = 13.1 |
| | | Hz, 1H), 3.44-3.31 (m, 2H), 3.14-3.06 (m, 1H), 2.07- |
| | | 2.01 (m, 1H), 1.50 (s, 9H), 1.24 (d, J = 6.8 Hz, 3H), 1.06- |
| | | 0.99 (m, 2H), 0.90-0.84 (m, 1H), 0.70-0.64 (m, 1H). |
| 206 | tert-butyl (R)-4-(5-cyclopropyl- | LC/MS ESI (m/z): 470 (M + H)⁺. ¹H NMR (400 MHz, |
| | 7-(2,5-difluorophenyl)-7H- | CDCl3) δ 8.42 (s, 1H), 7.44-7.37 (m, 1H), 7.24-7.19 (m, |
| | pyrrolo[2,3-d]pyrimidin-4-yl)-2- | 1H), 7.11-7.04 (m, 1H), 6.88 (d, J = 1.7 Hz, 1H), 4.55- |
| | methylpiperazine-1-carboxylate | 4.39 (m, 2H), 4.11 (d, J = 12.9 Hz, 1H), 3.97 (d, J = 13.2 |
| | | Hz, 1H), 3.44-3.31 (m, 2H), 3.14-3.06 (m, 1H), 2.06- |
| | | 2.00 (m, 1H), 1.50 (s, 9H), 1.23 (d, J = 6.8 Hz, 3H), 1.05- |
| | | 0.99 (m, 2H), 0.89-0.84 (m, 1H), 0.69-0.64 (m, 1H). |
| 207 | tert-butyl (R)-4-(7-(3-chloro-4,5- | LC/MS ESI (m/z): 504 (M + H)⁺. ¹H NMR (400 MHz, |
| | difluorophenyl)-5-cyclopropyl- | CDCl3) δ 8.43 (s, 1H), 7.63-7.57 (m, 1H), 7.56-7.51 (m, |
| | 7H-pyrrolo[2,3-d]pyrimidin-4- | 1H), 6.88 (s, 1H), 4.47 (d, J = 12.6 Hz, 1H), 4.44-4.37 (m, |
| | yl)-2-methylpiperazine-1- | 1H), 4.11 (d, J = 12.9 Hz, 1H), 3.97 (d, J = 13.2 Hz, 1H), |
| | carboxylate | 3.43-3.31 (m, 2H), 3.13-3.05 (m, 1H), 2.05-1.98 (m, |
| | | 1H), 1.50 (s, 9H), 1.22 (d, J = 6.7 Hz, 3H), 1.08-1.01 (m, |
| | | 2H), 0.91-0.84 (m, 1H), 0.70-0.63 (m, 1H). |
| 208 | tert-butyl (R)-4-(7-(3-chloro-4- | LC/MS ESI (m/z): 486 (M + H)⁺. ¹H NMR (400 MHz, |
| | fluorophenyl)-5-cyclopropyl- | CDCl3) δ 8.42 (s, 1H), 7.74-7.70 (m, 1H), 7.58-7.53 (m, |
| | 7H-pyrrolo[2,3-d]pyrimidin-4- | 1H), 7.28-7.24 (s, 1H), 6.89 (d, J = 0.8 Hz, 1H), 4.51- |
| | yl)-2-methylpiperazine-1- | 4.39 (m, 2H), 4.13-4.08 (m, 1H), 4.00-3.94 (m, 1H), |
| | carboxylate | 3.43-3.31 (m, 2H), 3.12-3.05 (m, 1H), 2.06-2.00 (m, |
| | | 1H), 1.50 (s, 9H), 1.22 (d, J = 6.8 Hz, 3H), 1.06-1.00 (m, |
| | | 2H), 0.89-0.84 (m, 1H), 0.68-0.63 (m, 1H). |
| 220 | tert-butyl (R)-4-(7-(3-chloro-2- | LC/MS ESI (m/z): 486 (M + H)⁺. ¹H NMR (400 MHz, |
| | fluorophenyl)-5-cyclopropyl- | CDCl3) δ 8.41 (s, 1H), 7.55-7.50 (m, 1H), 7.47-7.41 (m, |
| | 7H-pyrrolo[2,3-d]pyrimidin-4- | 1H), 7.25-7.20 (m, 1H), 6.87 (d, J = 1.6 Hz, 1H), 4.51 (d, |
| | yl)-2-methylpiperazine-1- | J = 12.6 Hz, 1H), 4.46-4.37 (m, 1H), 4.12 (d, J = 12.9 Hz, |
| | carboxylate | 1H), 3.97 (d, J = 13.2 Hz, 1H), 3.45-3.30 (m, 2H), 3.15- |
| | | 3.06 (m, 1H), 2.10-2.01 (m, 1H), 1.50 (s, 9H), 1.24 (d, J = |
| | | 6.7 Hz, 3H), 1.06-0.98 (m, 2H), 0.89-0.84 (m, 1H), 0.71- |
| | | 0.64 (m, 1H). |
| 224 | tert-butyl (R)-4-(7-(5-chloro-2- | LC/MS ESI (m/z): 486 (M + H)⁺. ¹H NMR (400 MHz, |
| | fluorophenyl)-5-cyclopropyl- | CDCl3) δ 8.42 (s, 1H), 7.62 (dd, J = 6.5, 2.6 Hz, 1H), 7.36- |
| | 7H-pyrrolo[2,3-d]pyrimidin-4- | 7.31 (m, 1H), 7.23-7.18 (m, 1H), 6.85 (d, J = 1.2 Hz, |
| | yl)-2-methylpiperazine-1- | 1H), 4.50 (d, J = 12.6 Hz, 1H), 4.45-4.38 (m, 1H), 4.12 (d, |
| | carboxylate | J = 12.9 Hz, 1H), 3.97 (d, J = 13.1 Hz, 1H), 3.43-3.31 (m, |
| | | 2H), 3.14-3.06 (m, 1H), 2.06-2.01 (m, 1H), 1.50 (s, 9H), |
| | | 1.23 (d, J = 6.7 Hz, 3H), 1.04-0.99 (m, 2H), 0.88-0.83 |
| | | (m, 1H), 0.69-0.64 (m, 1H). |
| 234 | tert-butyl 4-(5-cyclopropyl-7- | LC/MS ESI (m/z): 474 (M + H)⁺. ¹H NMR (400 MHz, |
| | (3,4,5-trifluorophenyl)-7H- | CDCl3) δ 8.45 (d, J = 8.2 Hz, 1H), 7.48-7.39 (m, 2H), |
| | pyrrolo[2,3-d]pyrimidin-4- | 6.87 (d, J = 7.8 Hz, 1H), 3.66 (d, J = 33.0 Hz, 8H), 2.08- |
| | yl)piperazine-1-carboxylate | 2.00 (m, 1H), 1.51 (d, J = 8.4 Hz, 9H), 1.07-1.01 (m, 2H), |
| | | 0.80-0.74 (m, 2H). |
| 204 | tert-butyl (R)-4-(5-cyclopropyl- | LC/MS ESI (m/z): 488 (M + H)⁺. ¹H NMR (400 MHz, |
| | 7-(3,4,5-trifluorophenyl)-7H- | CDCl3) δ 8.42 (s, 1H), 7.44 (dd, J = 8.7, 6.1 Hz, 2H), 6.87 |
| | pyrrolo[2,3-d]pyrimidin-4-yl)-2- | (d, J = 0.8 Hz, 1H), 4.50-4.38 (m, 2H), 4.10 (d, J = 13.0 |
| | methylpiperazine-1-carboxylate | Hz, 1H), 3.96 (d, J = 13.1 Hz, 1H), 3.42-3.31 (m, 2H), |
| | | 3.13-3.05 (m, 1H), 2.05-1.99 (m, 1H), 1.50 (s, 9H), 1.21 |
| | | (d, J = 6.8 Hz, 3H), 1.07-1.00 (m, 2H), 0.90-0.84 (m, |
| | | 1H), 0.69-0.63 (m, 1H). |
| 102 | tert-butyl 4-(7-(3-chlorophenyl)- | LC/MS ESI (m/z): 491 (M + H)⁺. ¹H NMR (400 MHz, |
| | 5-(pyridin-3-yl)-7H-pyrrolo [2,3- | CDCl3) δ 8.86-8.82 (m, 1H), 8.62 (dd, J = 4.8, 1.6 Hz, |
| | d]pyrimidin-4-yl)piperazine-1- | 1H), 8.56 (s, 1H), 7.91-7.86 (m, 1H), 7.82-7.76 (m, 1H), |
| | carboxylate | 7.70-7.65 (m, 1H), 7.51-7.46 (m, 1H), 7.43-7.37 (m, |
| | | 3H), 3.28 (s, 8H), 1.43 (s, 9H). |

### Example 36. Synthesis of ethyl (S)-4-(5-(2-fluorophenyl)-7-(5-methoxypyridin-3-yl)-7H-pyrrolo[2,3-d]pyrimidin-4-yl)-3-methylpiperazine-1-carboxylate (Compound 126)

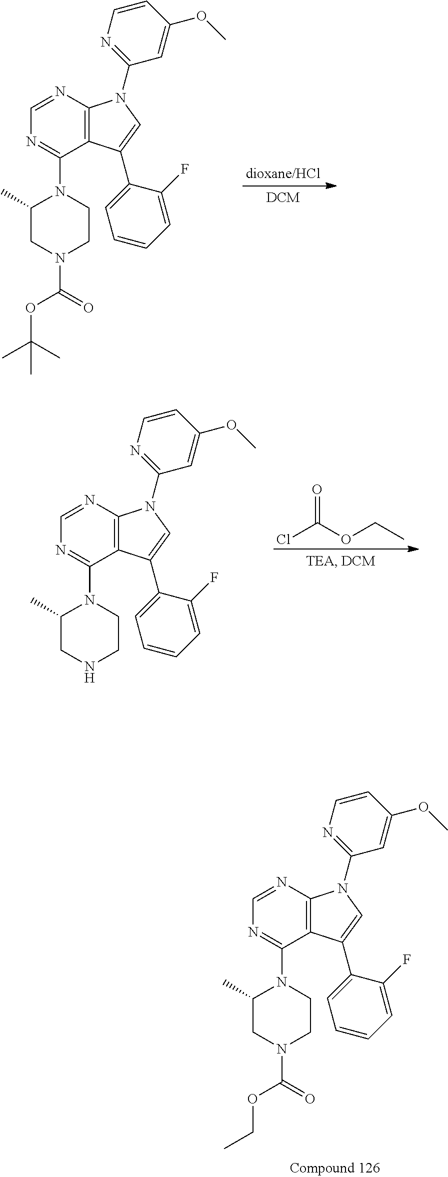

### Step 1. (S)-5-(2-Fluorophenyl)-7-(5-methoxypyridin-3-yl)-4-(2-methylpiperazin-1-yl)-7H-pyrrolo[2,3-d]pyrimidine

To a solution of tert-butyl (S)-4-(5-(2-fluorophenyl)-7-(5-methoxypyridin-3-yl)-7H-pyrrolo[2,3-d]pyrimidin-4-yl)-3-methylpiperazine-1-carboxylate (80 mg, 0.16 mmol, prepared following an analogous procedure described for compound 1004 in DCM (3 mL) was added HCl (4.0 mL, 4.0 M in dioxane). The resulting mixture was stirred at room temperature for 0.5 h. After removal of solvent, the residue was diluted with DCM and washed with NaHCO₃ (aq.). The organic layer was extracted twice with DCM. The combined organic layers were washed with brine, dried over Na₂SO₄, filtered and concentrated. The residue was used in the next step directly. (S)-5-(2-fluorophenyl)-7-(5-methoxypyridin-3-yl)-4-(2-methylpiperazin-1-yl)-7H-pyrrolo[2,3-d]pyrimidine (40 mg, 63%). LC/MS ESI (m/z): 419 (M+H)⁺.

### Step 2. Ethyl (S)-4-(5-(2-fluorophenyl)-7-(5-methoxypyridin-3-yl)-7H-pyrrolo[2,3-d]pyrimidin-4-yl)-3-methylpiperazine-1-carboxylate

To a solution of (S)-5-(2-fluorophenyl)-7-(5-methoxypyridin-3-yl)-4-(2-methylpiperazin-1-yl)-7H-pyrrolo[2,3-d]pyrimidine (40 mg, 0.10 mmol) in DCM (3 mL) at 0° C. was added TEA (0.30 mL, 0.30 mmol), followed by ethyl carbonochloridate (0.020 mL, 0.20 mmol) dropwise. The resulting mixture was stirred at room temperature overnight. The reaction was quenched with water and extracted twice with DCM. The combined organic layers were washed with NaHCO₃ (aq.), dried over Na₂SO₄, filtered and concentrated. The residue was purified by flash column chromatography (silica gel, 0˜30%, ethyl acetate in petroleum ether) to give crude product which was further purified by prep-HPLC to afford ethyl (S)-4-(5-(2-fluorophenyl)-7-(5-methoxypyridin-3-yl)-7H-pyrrolo[2,3-d]pyrimidin-4-yl)-3-methylpiperazine-1-carboxylate (31 mg, 63%) as a white solid. LC/MS ESI (m/z): 491 (M+H)⁺. ¹H NMR (400 MHz, DMSO-d6) δ 8.77 (d, J=2.0 Hz, 1H), 8.45 (s, 1H), 8.33 (d, J=2.6 Hz, 1H), 8.05 (s, 1H), 7.94 (t, J=2.3 Hz, 1H), 7.56 (td, J=7.5, 1.5 Hz, 1H), 7.50-7.45 (m, 1H), 7.40-7.35 (m, 2H), 4.19-4.14 (m, 1H), 4.04-3.98 (m, 2H), 3.92 (s, 3H), 3.66-3.60 (m, 1H), 3.50-3.41 (m, 3H), 3.03-2.96 (m, 1H), 2.74-2.68 (m, 1H), 1.14 (t, J=7.0 Hz, 3H), 0.91 (d, J=6.6 Hz, 3H).

### Example 37. Synthesis of 1,1,1-trifluoro-2-methylpropan-2-yl (S)-4-(7-(3-chlorophenyl)-5-cyclopropyl-7H-pyrrolo[2,3-d]pyrimidin-4-yl)-3-methylpiperazine-1-carboxylate (Compound 140)

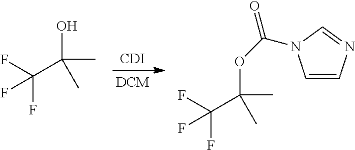

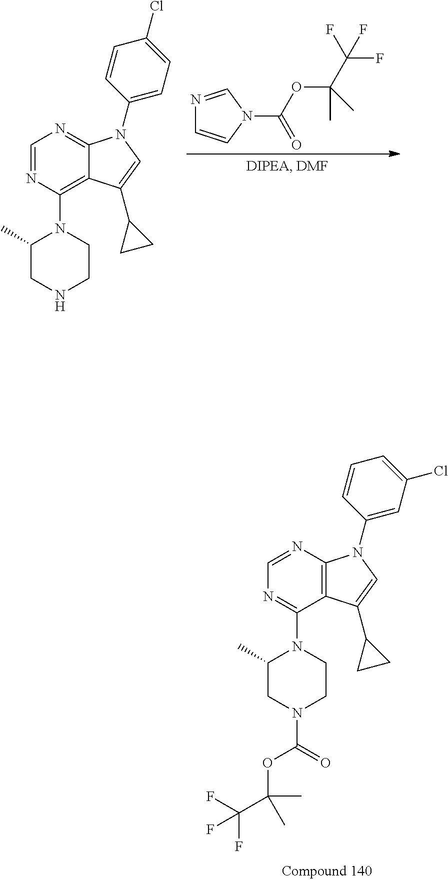

### Step 1. 2,2,2-Trifluoroethyl 1H-imidazole-1-carboxylate

To a solution of 1,1,1-trifluoro-2-methylpropan-2-ol (1.0 g, 0.78 mmol) in DCM (10 mL) was added CDI (1.4 g, 0.86 mmol). The resulting mixture was stirred at room temperature overnight. The reaction was quenched with water and extracted twice with DCM. The combined organic layers were washed with brine, dried over Na₂SO₄, filtered and concentrated to afford 1,1,1-trifluoro-2-methylpropan-2-yl 1H-imidazole-1-carboxylate (1.1 g, 64%) as a white solid. LC/MS ESI (m/z): 223 (M+H)⁺.

### Step 2. 1,1,1-Trifluoro-2-methylpropan-2-yl (S)-4-(7-(3-chlorophenyl)-5-cyclopropyl-7H-pyrrolo[2,3-d]pyrimidin-4-yl)-3-methylpiperazine-1-carboxylate

To a solution of (S)-7-(4-chlorophenyl)-5-cyclopropyl-4-(2-methylpiperazin-1-yl)-7H-pyrrolo[2,3-d]pyrimidine (70 mg, 0.19 mmol, prepared following the procedures described for compound 461) in DMF (5 mL) were added 1,1,1-trifluoro-2-methylpropan-2-yl 1H-imidazole-1-carboxylate (84 mg, 0.38 mmol) and DIPEA (74 mg, 0.57 mmol). The resulting mixture was stirred at 80° C. overnight under a N₂ atmosphere. The reaction was quenched with water and extracted twice with EtOAc. The combined organic layers were washed with brine, dried over Na₂SO₄, filtered and concentrated. The residue was purified by flash column chromatography (silica gel, 0-50%, ethyl acetate in petroleum ether) to give crude product which was further purified by prep-HPLC to afford 1,1,1-trifluoro-2-methylpropan-2-yl (S)-4-(7-(3-chlorophenyl)-5-cyclopropyl-7H-pyrrolo[2,3-d]pyrimidin-4-yl)-3-methylpiperazine-1-carboxylate (29 mg, 29% yield) as a white solid. LC/MS ESI (m/z): 522 (M+H)⁺. ¹H NMR (400 MHz, CDCl₃) δ 8.45 (s, 1H), 7.68 (t, J=2.0 Hz, 1H), 7.62-7.57 (m, 1H), 7.42 (t, J=8.1 Hz, 1H), 7.33-7.29 (m, 1H), 6.93 (s, 1H), 4.79-4.71 (m, 1H), 4.15-3.79 (m, 3H), 3.57 (t, J=12.6 Hz, 1H), 3.43-3.32 (m, 1H), 3.30-3.11 (m, 1H), 2.08-2.00 (m, 1H), 1.73 (d, J=7.2 Hz, 6H), 1.24 (d, J=6.6 Hz, 3H), 1.05-0.99 (m, 2H), 0.84-0.77 (m, 1H), 0.75-0.68 (m, 1H).

The following compounds were prepared by a procedure analogous to the synthesis of compound 140 from the corresponding amines.

| Cmpd | | |
|---|---|---|
| No. | Chemical Name | LCMS and ¹H NMR |
| 141 | 1,1,1-trifluoro-2- | LC/MS ESI (m/z): 522 (M + H)⁺. ¹H NMR (400 MHz, |
| | methylpropan-2-yl (R)-4-(7-(4- | CDCl₃) δ8.43 (s, 1H), 7.62-7.58 (m, 2H), 7.48-7.44 (m, |
| | chlorophenyl)-5-cyclopropyl- | 2H), 6.91 (s, 1H), 4.79-4.71 (m, 1H), 4.15-3.78 (m, 3H), |
| | 7H-pyrrolo[2,3-d]pyrimidin-4- | 3.57 (t, J = 12.4 Hz, 1H), 3.43-3.10 (m, 2H), 2.08-2.00 |
| | yl)-3-methylpiperazine-1- | (m, 1H), 1.73 (d, J = 6.9 Hz, 6H), 1.24 (d, J = 6.6 Hz, 3H), |
| | carboxylate | 1.04-0.99 (m, 2H), 0.82-0.77 (m, 1H), 0.74-0.66 (m, |
| | | 1H). |
| 148 | 1,1,1-trifluoro-2- | LC/MS ESI (m/z): 522 (M + H)⁺. ¹H NMR (400 MHz, |
| | methylpropan-2-yl (S)-4-(7-(4- | CDCl₃) δ8.43 (s, 1H), 7.60 (d, J = 8.8 Hz, 2H), 7.46 (d, J = |
| | chlorophenyl)-5-cyclopropyl- | 8.8 Hz, 2H), 6.91 (s, 1H), 4.79-4.71 (m, 1H), 4.15-3.79 |
| | 7H-pyrrolo[2,3-d]pyrimidin-4- | (m, 3H), 3.61-3.52 (m, 1H), 3.43-3.11 (m, 2H), 2.07- |
| | yl)-3-methylpiperazine-1- | 1.99 (m, 1H), 1.73 (d, J = 6.8 Hz, 6H), 1.24 (d, J = 6.6 Hz, |
| | carboxylate | 3H), 1.03-0.99 (m, 2H), 0.82-0.76 (m, 1H), 0.74-0.67 |
| | | (m, 1H). |

### Example 38. Synthesis of ethyl (S)-4-(5-cyclopropyl-7-(5-methylpyridin-3-yl)-7H-pyrrolo[2,3-d]pyrimidin-4-yl)-3-methylpiperazine-1-carboxylate (Compound 153)

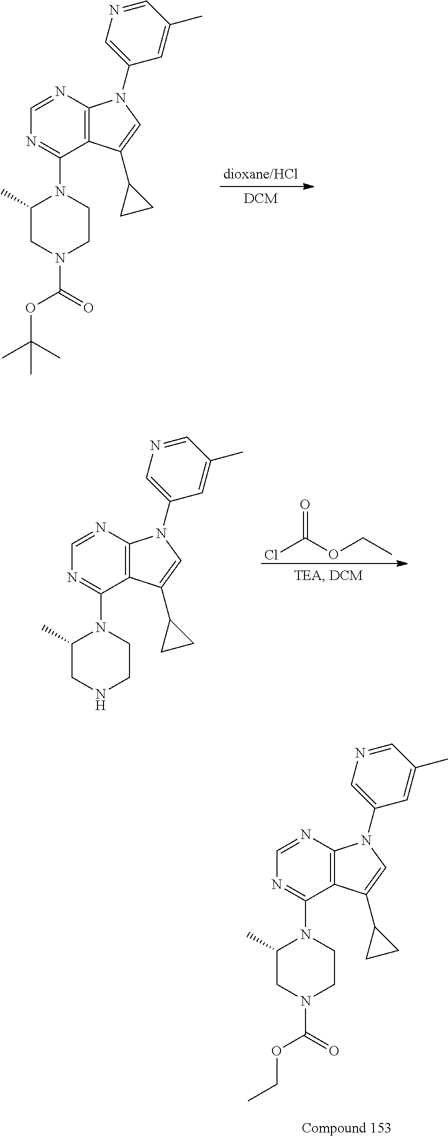

### Step 1. (S)-5-Cyclopropyl-4-(2-methylpiperazin-1-yl)-7-(5-methylpyridin-3-yl)-7H-pyrrolo[2,3-d]pyrimidine

To a solution of tert-butyl (S)-4-(5-cyclopropyl-7-(5-methylpyridin-3-yl)-7H-pyrrolo[2,3-d]pyrimidin-4-yl)-3-methylpiperazine-1-carboxylate (80 mg, 0.16 mmol, prepared following an analogous procedure described for compound 259) in DCM (3 mL) was added HCl (4.0 mL, 4.0 M in dioxane). The resulting mixture was stirred at room temperature for 0.5 h. After removal of solvent, the residue was diluted with DCM and washed with NaHCO₃ (aq.). The aqueous layer was extracted twice with DCM. The combined organic layers were washed with brine, dried over Na₂SO₄, filtered and concentrated. The residue was used in the next step directly. (S)-5-cyclopropyl-4-(2-methylpiperazin-1-yl)-7-(5-methylpyridin-3-yl)-7H-pyrrolo[2,3-d]pyrimidine (40 mg, 63%). LC/MS ESI (m/z): 349 (M+H)⁺.

### Step 2. Ethyl (S)-4-(5-cyclopropyl-7-(5-methylpyridin-3-yl)-7H-pyrrolo[2,3-d]pyrimidin-4-yl)-3-methylpiperazine-1-carboxylate

To a solution of (S)-5-cyclopropyl-4-(2-methylpiperazin-1-yl)-7-(5-methylpyridin-3-yl)-7H-pyrrolo[2,3-d]pyrimidine (40 mg, 0.10 mmol) in DCM (3 mL) at 0° C. was added TEA (0.30 mL, 0.30 mmol), followed by ethyl carbonochloridate (0.020 mL, 0.20 mmol) dropwise. The resulting mixture was stirred at room temperature overnight. The reaction was quenched with water and extracted twice with DCM. The combined organic layers were washed with NaHCO₃ (aq.), dried over Na₂SO₄, filtered and concentrated. The residue was purified by flash column chromatography (silica gel, 0˜30%, ethyl acetate in petroleum ether) to give crude product which was further purified by prep-HPLC to afford ethyl (S)-4-(5-cyclopropyl-7-(5-methylpyridin-3-yl)-7H-pyrrolo[2,3-d]pyrimidin-4-yl)-3-methylpiperazine-1-carboxylate (31 mg, 63%) as a white solid. LC/MS ESI (m/z): 421 (M+H)⁺. ¹H NMR (400 MHz, DMSO-d6) δ 8.86 (d, J=2.4 Hz, 1H), 8.39 (d, J=1.1 Hz, 1H), 8.36 (s, 1H), 8.08 (s, 1H), 7.54 (s, 1H), 4.74-4.67 (m, 1H), 4.14-4.07 (m, 2H), 4.03-3.91 (m, 2H), 3.81 (d, J=12.9 Hz, 1H), 3.50-3.39 (m, 2H), 3.18-3.11 (m, 1H), 2.39 (s, 3H), 2.06-2.00 (m, 1H), 1.22 (t, J=7.2 Hz, 3H), 1.14 (d, J=6.6 Hz, 3H), 1.01-0.96 (m, 2H), 0.90-0.86 (m, 1H), 0.81-0.77 (m, 1H).

### Example 39. Synthesis of ethyl (S)-4-(7-(3-chloro-4-fluorophenyl)-5-(pyridin-2-yl)-7H-pyrrolo[2,3-d]pyrimidin-4-yl)-3-methylpiperazine-1-carboxylate (Compound 155)

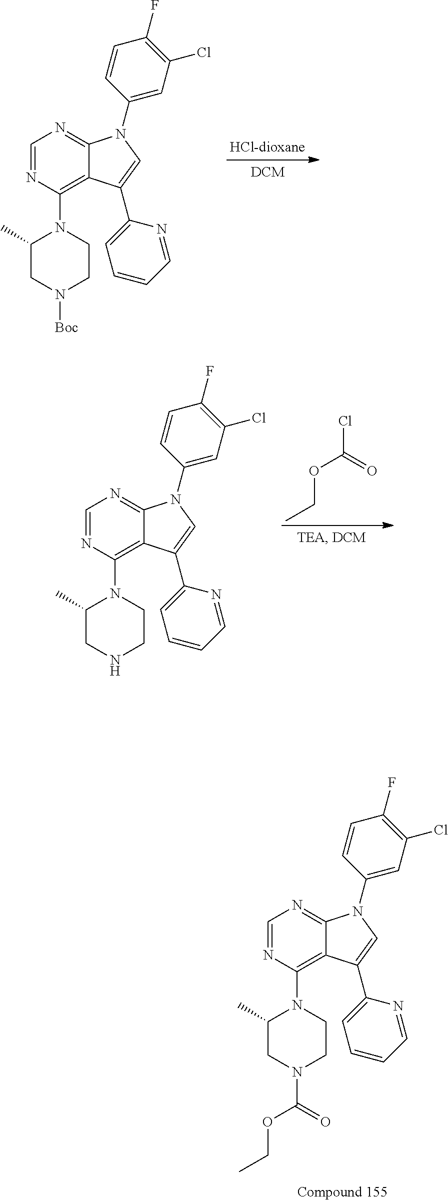

### Step 1. (S)-7-(3-Chloro-4-fluorophenyl)-4-(2-methylpiperazin-1-yl)-5-(pyridin-2-yl)-7H-pyrrolo[2,3-d]pyrimidine

To a solution of tert-butyl (S)-4-(7-(3-chloro-4-fluorophenyl)-5-(pyridin-2-yl)-7H-pyrrolo[2,3-d]pyrimidin-4-yl)-3-methylpiperazine-1-carboxylate (400 mg, 0.77 mmol, prepared following the procedures described for compound 154 in DCM (5 mL) was added HCl (3.0 mL, 4.0 M in dioxane). The resulting mixture was stirred at room temperature for 2 h. After removal of solvent, the residue was diluted with DCM and washed with NaHCO₃ (aq.). The organic layer was extracted twice with DCM. The combined organic layers were washed with brine, dried over Na₂SO₄, filtered and concentrated. The residue was used in the next step directly. (S)-7-(3-chloro-4-fluorophenyl)-4-(2-methylpiperazin-1-yl)-5-(pyridin-2-yl)-7H-pyrrolo[2,3-d]pyrimidine (280 mg). LC/MS ESI (m/z): 423 (M+H)⁺.

### Step 2. Ethyl (S)-4-(7-(3-chloro-4-fluorophenyl)-5-(pyridin-2-yl)-7H-pyrrolo[2,3-d]pyrimidin-4-yl)-3-methylpiperazine-1-carboxylate

To a solution of (S)-7-(3-chloro-4-fluorophenyl)-4-(2-methylpiperazin-1-yl)-5-(pyridin-2-yl)-7H-pyrrolo[2,3-d]pyrimidine (97 mg, 0.23 mmol) in DCM (5 mL) at 0° C. was added TEA (70 mg, 0.69 mmol), followed by ethyl carbonochloridate (75 mg, 0.69 mmol) dropwise. The resulting mixture was stirred at room temperature overnight. The reaction was quenched with water and extracted twice with DCM. The combined organic layers were washed with NaHCO₃ (aq), dried over Na₂SO₄, filtered and concentrated. The residue was purified by flash column chromatography (silica gel, 0˜30%, ethyl acetate in petroleum ether) to give crude product which was further purified by prep-HPLC to afford ethyl (S)-4-(7-(3-chloro-4-fluorophenyl)-5-(pyridin-2-yl)-7H-pyrrolo[2,3-d]pyrimidin-4-yl)-3-methylpiperazine-1-carboxylate (41 mg, 36%) as a white solid. LC/MS ESI (m/z): 495 (M+H)⁺. ¹H NMR (400 MHz, CDCl3) δ 8.69 (d, J=4.1 Hz, 1H), 8.52 (s, 1H), 7.85 (dd, J=6.4, 2.6 Hz, 1H), 7.79 (td, J=7.7, 1.8 Hz, 1H), 7.67-7.63 (m, 2H), 7.59 (d, J=7.8 Hz, 1H), 7.31 (t, J=8.7 Hz, 1H), 7.28-7.24 (m, 1H), 4.39-4.23 (m, 1H), 4.16-4.08 (m, 2H), 3.98-3.83 (m, 1H), 3.62 (dd, J=26.8, 12.6 Hz, 2H), 3.22-3.13 (m, 1H), 3.09-2.79 (m, 2H), 1.24 (t, J=7.1 Hz, 3H), 1.05 (d, J=6.3 Hz, 3H).

The following compounds were prepared by procedures analogous to the synthesis of compound 155 from the corresponding Boc-protected amines.

| Cmpd | | |
|---|---|---|
| No. | Chemical Name | LCMS and ¹H NMR |
| 118 | Ethyl (2R,5S)-4-(7-(3- | LC/MS ESI (m/z): 491 (M + H)^{+ 1}H NMR(400 MHz, |
| | chlorophenyl)-5-(pyridin-2-yl)- | CDCl3) δ 8.69 (d, J = 4.2 Hz, 1H), 8.54 (s, 1H), 7.85-7.76 |
| | 7H-pyrrolo[2,3-d]pyrimidin-4- | (m, 2H), 7.72-7.68 (m, 2H), 7.63-7.59 (m, 1H), 7.50- |
| | yl)-2,5-dimethylpiperazine-1- | 7.44 (m, 1H), 7.39-7.34 (m, 1H), 7.30-7.24 (m, 1H), |
| | carboxylate | 4.32-4.06 (m, 4H), 3.51-3.40 (m, 2H), 3.36-3.29 (m, |
| | | 1H), 3.21-3.06 (m, 1H), 1.24 (t, 3H), 1.13 (d, J = 6.6 Hz, |
| | | 3H), 1.01 (d, J = 6.6 Hz, 3H). |
| 125 | Ethyl (2R,5S)-4-(7-(3- | LC/MS ESI (m/z): 492 (M + H)⁺. ¹H NMR (400 MHz, |
| | chlorophenyl)-5-(pyrazin-2-yl)- | CDCl3) δ 8.93 (d, J = 1.3 Hz, 1H), 8.65-8.61 (m, 1H), |
| | 7H-pyrrolo[2,3-d]pyrimidin-4- | 8.57 (s, 1H), 8.53 (d, J = 2.5 Hz, 1H), 7.87-7.76 (m, 2H), |
| | yl)-2,5-dimethylpiperazine-1- | 7.72-7.68 (m, 1H), 7.51-7.47 (m, 1H), 7.41-7.37 (m, |
| | carboxylate | 1H), 4.35-4.08 (m, 4H), 3.53-3.37 (m, 3H), 3.31-3.12 |
| | | (m, 1H), 1.24 (t, 3H), 1.16 (d, J = 6.7 Hz, 3H), 1.05 (d, J = |
| | | 6.6 Hz, 3H). |

### Example 40. Synthesis of tert-butyl (R)-4-(7-(3-chlorophenyl)-5-cyclopentyl-7H-pyrrolo[2,3-d]pyrimidin-4-yl)-3-methylpiperazine-1-carboxylate (Compound 157)

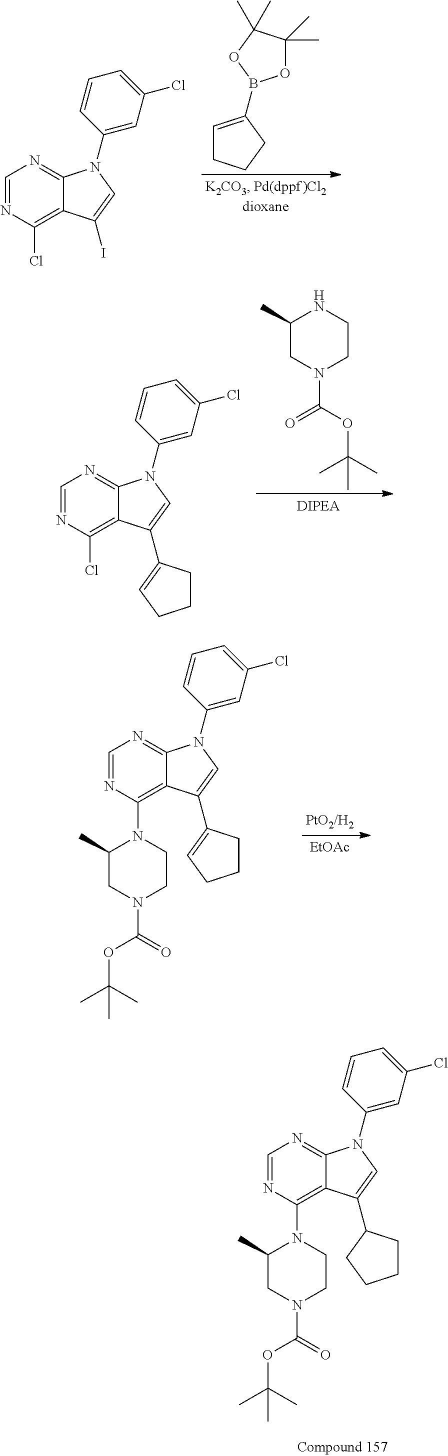

### Step 1. 4-Chloro-7-(3-chlorophenyl)-5-(cyclopent-1-en-1-yl)-7H-pyrrolo[2,3-d]pyrimidine

To a solution of 4-chloro-7-(3-chlorophenyl)-5-iodo-7H-pyrrolo[2,3-d]pyrimidine (1.0 g, 2.6 mmol, prepared following step 1 of the procedures described for compound 274) in dioxane-H₂O (15 mL, 5:1 v/v) were added 2-(cyclopent-1-en-1-yl)-4,4,5,5-tetramethyl-1,3,2-dioxaborolane (450 mg, 2.3 mmol), K₂CO₃ (1.1 g, 7.8 mmol) and Pd(dppf)Cl₂ (190 mg, 0.26 mmol). The resulting mixture was heated to 90° C. overnight. After cooling to room temperature, the reaction was filtered and the filtrate was partitioned between EtOAc and water. The aqueous phase was extracted twice with EtOAc. The combined organic layers were washed with brine, dried over Na₂SO₄, filtered and concentrated. The residue was purified by flash chromatography (silica gel, 0˜40% EtOAc in petroleum ether) to give 300 mg of 4-chloro-7-(3-chlorophenyl)-5-(cyclopent-1-en-1-yl)-7H-pyrrolo[2,3-d]pyrimidine (36% yield) as a solid. LC/MS ESI (m/z): 330 (M+H)⁺.

### Step 2. tert-Butyl (R)-4-(7-(3-chlorophenyl)-5-(cyclopent-1-en-1-yl)-7H-pyrrolo[2,3-d]pyrimidin-4-yl)-3-methylpiperazine-1-carboxylate

A mixture of 4-chloro-7-(3-chlorophenyl)-5-(cyclopent-1-en-1-yl)-7H-pyrrolo[2,3-d]pyrimidine (100 mg, 0.30 mmol) and tert-butyl (R)-3-methylpiperazine-1-carboxylate (120 mg, 0.60 mmol) in DIPEA (0.5 mL) was stirred at 140° C. for 3 h. After cooling to room temperature, the DIPEA was removed by vacuum. The residue was purified by flash chromatography (silica gel, 0˜40% EtOAc in petroleum ether) to afford tert-butyl (R)-4-(7-(3-chlorophenyl)-5-(cyclopent-1-en-1-yl)-7H-pyrrolo[2,3-d]pyrimidin-4-yl)-3-methylpiperazine-1-carboxylate (110 mg, 73%) as a white solid. LC/MS ESI (m/z): 494 (M+H)⁺.

### Step 3. tert-Butyl (R)-4-(7-(3-chlorophenyl)-5-cyclopentyl-7H-pyrrolo[2,3-d]pyrimidin-4-yl)-3-methylpiperazine-1-carboxylate

A mixture of tert-butyl (R)-4-(7-(3-chlorophenyl)-5-(cyclopent-1-en-1-yl)-7H-pyrrolo[2,3-d]pyrimidin-4-yl)-3-methylpiperazine-1-carboxylate (110 mg, 0.22 mmol), PtO₂ (51 mg) in EtOAc (8 mL) was stirred at room temperature for overnight under H₂ atmosphere (˜1 atm). The reaction was filtered, and the filtrate was concentrated. The residue was purified by flash chromatography (silica gel, 0˜50% EtOAc in petroleum ether) and prep-HPLC to give tert-butyl (R)-4-(7-(3-chlorophenyl)-5-cyclopentyl-7H-pyrrolo[2,3-d]pyrimidin-4-yl)-3-methylpiperazine-1-carboxylate (38 mg, 35% yield) as a white solid. LCMS ESI (m/z): 496. (M+H)⁺. ¹H NMR (400 MHz, CDCl₃) δ 8.51 (s, 1H), 7.72 (t, J=1.9 Hz, 1H), 7.68-7.63 (m, 1H), 7.44 (t, J=8.1 Hz, 1H), 7.31 (d, J=9.0 Hz, 1H), 7.13 (s, 1H), 4.18 (s, 1H), 3.91 (s, 1H), 3.65 (s, 1H), 3.48 (s, 3H), 3.33-3.19 (m, 2H), 2.28 (s, 1H), 2.15 (s, 1H), 1.90-1.63 (m, 6H), 1.50 (s, 9H), 1.18 (d, J=6.1 Hz, 3H).

The following compound was prepared by procedures analogous to the synthesis of compound 157 from the corresponding amine.

| Cmpd | | |
|---|---|---|
| No. | Chemical Name | LCMS and ¹H NMR |
| 158 | tert-Butyl (S)-4-(7-(3- | LC/MS ESI (m/z): 496 (M + H)⁺. ¹H NMR (400 MHz, |
| | chlorophenyl)-5-cyclopentyl- | CDCl3) δ 8.51 (s, 1H), 7.72 (t, J = 2.0 Hz, 1H), 7.66-7.63 |
| | 7H-pyrrolo[2,3-d]pyrimidin-4- | (m, 1H), 7.44 (t, J = 8.1 Hz, 1H), 7.33-7.29 (m, 1H), 7.13 |
| | yl)-3-methylpiperazine-1- | (s, 1H), 4.22-4.15 (m, 1H), 3.91 (s, 1H), 3.65 (s, 1H), 3.48 |
| | carboxylate | (s, 3H), 3.33-3.19 (m, 2H), 2.33-2.26 (m, 1H), 2.20- |
| | | 2.12 (m, 1H), 1.89-1.65 (m, 6H), 1.50 (s, 9H), 1.18 (d, J = |
| | | 6.1 Hz, 3H). |

### Example 41. Synthesis of tert-butyl (2R,5S)-2,5-dimethyl-4-(5-phenyl-7-(pyridin-3-yl)-7H-pyrrolo[2,3-d]pyrimidin-4-yl)piperazine-1-carboxylate (Compound 169)

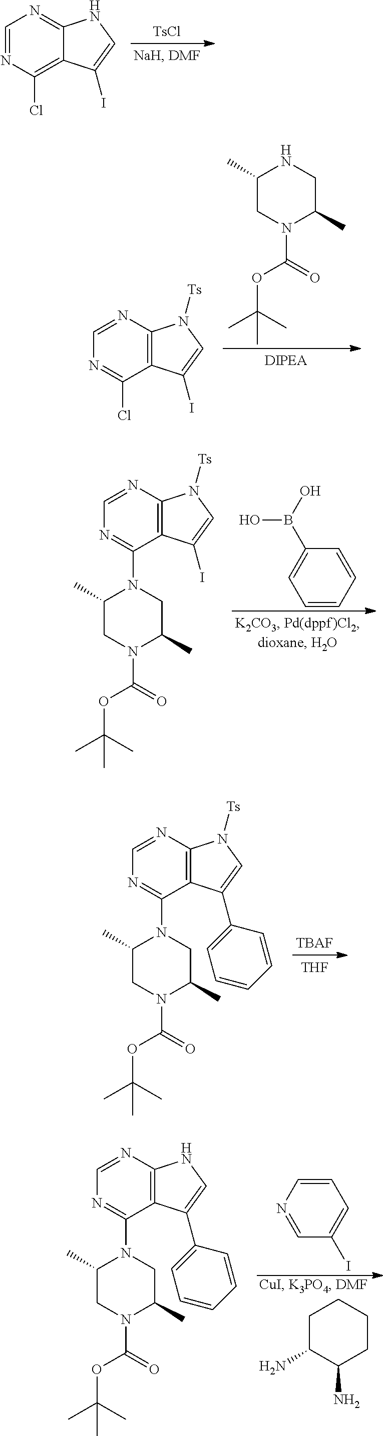

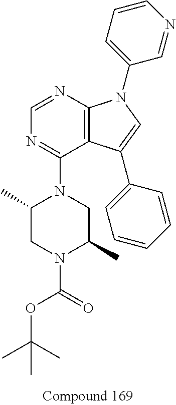 [SMILES: C[C@@H]1CN(c2ncnc3c2c(-c2ccccc2)cn3-c2cccnc2)[C@@H](C)CN1C(=O)OC(C)(C)C]

### Step 1. 4-Chloro-5-iodo-7-tosyl-7H-pyrrolo[2,3-d]pyrimidine

To a suspension of NaH (1.0 g, 27 mmol, 60% wt %) in anhydrous DMF (60 mL) at 0° C. was added 4-chloro-5-iodo-7H-pyrrolo[2,3-d]pyrimidine (5.0 g, 18 mmol) in portions. The resulting mixture was stirred at 0° C. for 30 minutes before TosCl (3.4 g, 18 mmol) was added in portions. After the addition, the reaction was stirred at room temperature overnight. The reaction was poured into ice water, filtered. The solid was collected and further dried in vacuo to give 4-chloro-5-iodo-7-tosyl-7H-pyrrolo[2,3-d]pyrimidine (6.0 g, 77%) as a white solid. LCMS ESI (m/z): 434 (M+H)⁺.

### Step 2. tert-Butyl (2R,5S)-4-(5-iodo-7-tosyl-7H-pyrrolo[2,3-d]pyrimidin-4-yl)-2,5-dimethylpiperazine-1-carboxylate

To a solution of 4-chloro-5-iodo-7-tosyl-7H-pyrrolo[2,3-d]pyrimidine (4.0 g, 9.2 mmol) in DIPEA (5.0 mL, 28 mmol) was added tert-butyl (2R,5S)-2,5-dimethylpiperazine-1-carboxylate (2.0 g, 9.2 mmol). The resulting mixture was heated to 150° C. for 3 h under a N₂ atmosphere. After cooling to room temperature, solvent was removed and the residue was purified by flash column chromatography (silica gel, 0˜30%, ethyl acetate in petroleum ether) to afford tert-butyl (2R,5S)-4-(5-iodo-7-tosyl-7H-pyrrolo[2,3-d]pyrimidin-4-yl)-2,5-dimethylpiperazine-1-carboxylate (2.5 g, 43%) as a yellow solid. LC/MS ESI (m/z): 612 (M+H)⁺.

### Step 3. tert-Butyl (2R,5S)-2,5-dimethyl-4-(5-phenyl-7-tosyl-7H-pyrrolo[2,3-d]pyrimidin-4-yl)piperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (150 mg, 0.30 mmol) in dioxane (10 mL) and H₂O (1 mL) were added phenylboronic acid (60 mg, 0.60 mmol), K₂CO₃ (100 mg, 0.75 mmol) and Pd(dppf)Cl₂ (18 mg, 0.010 mmol). The resulting mixture was stirred at 90° C. overnight under a N₂ atmosphere. After cooling to room temperature, solvent was removed and the residue was purified by flash column chromatography (silica gel, 0˜20%, ethyl acetate in petroleum ether) to afford tert-butyl (2R,5S)-2,5-dimethyl-4-(5-phenyl-7-tosyl-7H-pyrrolo[2,3-d]pyrimidin-4-yl)piperazine-1-carboxylate (100 mg, 72%) as a yellow solid. LC/MS ESI (m/z): 562 (M+H)⁺.

### Step 4. tert-Butyl (2R,5S)-2,5-dimethyl-4-(5-phenyl-7H-pyrrolo[2,3-d]pyrimidin-4-yl)piperazine-1-carboxylate

To a solution of tert-butyl (2R,5S)-2,5-dimethyl-4-(5-phenyl-7-tosyl-7H-pyrrolo[2,3-d]pyrimidin-4-yl)piperazine-1-carboxylate (100 mg, 0.18 mmol) in THE (2 mL) was added TBAF (5 mL). The resulting mixture was stirred at room temperature overnight. The reaction was quenched with water and extracted twice with EtOAc. The combined organic layers were washed with brine, dried over Na₂SO₄, filtered and concentrated. The residue was purified by flash column chromatography (silica gel, 0˜60%, ethyl acetate in petroleum ether) to afford tert-butyl (2R,5S)-2,5-dimethyl-4-(5-phenyl-7H-pyrrolo[2,3-d]pyrimidin-4-yl)piperazine-1-carboxylate (50 mg, 69%) as a white solid. LC/MS ESI (m/z): 408 (M+H)⁺.

### Step 5. tert-Butyl (2R,5S)-2,5-dimethyl-4-(5-phenyl-7-(pyridin-3-yl)-7H-pyrrolo[2,3-d]pyrimidin-4-yl)piperazine-1-carboxylate

To a solution of tert-butyl (2R,5S)-2,5-dimethyl-4-(5-phenyl-7H-pyrrolo[2,3-d]pyrimidin-4-yl)piperazine-1-carboxylate (50 mg, 0.12 mmol) in DMF (10 mL) were added 3-iodopyridine (50 mg, 0.24 mmol), trans-cyclohexane-1,2-diamine (15 mg, 0.12 mmol), CuI (25 mg, 0.12 mmol) and K₃PO₄ (280 mg, 1.2 mmol). The resulting mixture was heated to 100° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to afford tert-butyl (2R,5S)-2,5-dimethyl-4-(5-phenyl-7-(pyridin-3-yl)-7H-pyrrolo[2,3-d]pyrimidin-4-yl)piperazine-1-carboxylate (50 mg, 85%) as a white solid, which was further purified by prep-HPLC to afford 29 mg of white solid. LC/MS ESI (m/z): 485 (M+H)⁺. ¹H NMR (400 MHz, MeOD) δ 9.09 (s, 1H), 8.58 (d, J=4.3 Hz, 1H), 8.41 (s, 1H), 8.33 (ddd, J=8.3, 2.5, 1.4 Hz, 1H), 7.73 (s, 1H), 7.64 (dd, J=8.3, 4.8 Hz, 1H), 7.61-7.58 (m, 2H), 7.51 (t, J=7.6 Hz, 2H), 7.41 (t, J=7.4 Hz, 1H), 4.24-4.10 (m, 2H), 3.47-3.42 (m, 1H), 3.29-3.21 (m, 2H), 2.97-2.80 (m, 1H), 1.43 (s, 9H), 1.14 (d, J=6.8 Hz, 3H), 0.91 (d, J=6.7 Hz, 3H).

### Example 42. Synthesis of tert-butyl (S)-4-(5-cyclopropyl-7-(5-methylpyridin-3-yl)-7H-pyrrolo[2,3-d]pyrimidin-4-yl)-2-methylpiperazine-1-carboxylate (Compound 174) and tert-butyl (R)-4-(5-cyclopropyl-7-(5-methylpyridin-3-yl)-7H-pyrrolo[2,3-d]pyrimidin-4-yl)-2-methylpiperazine-1-carboxylate (Compound 175)

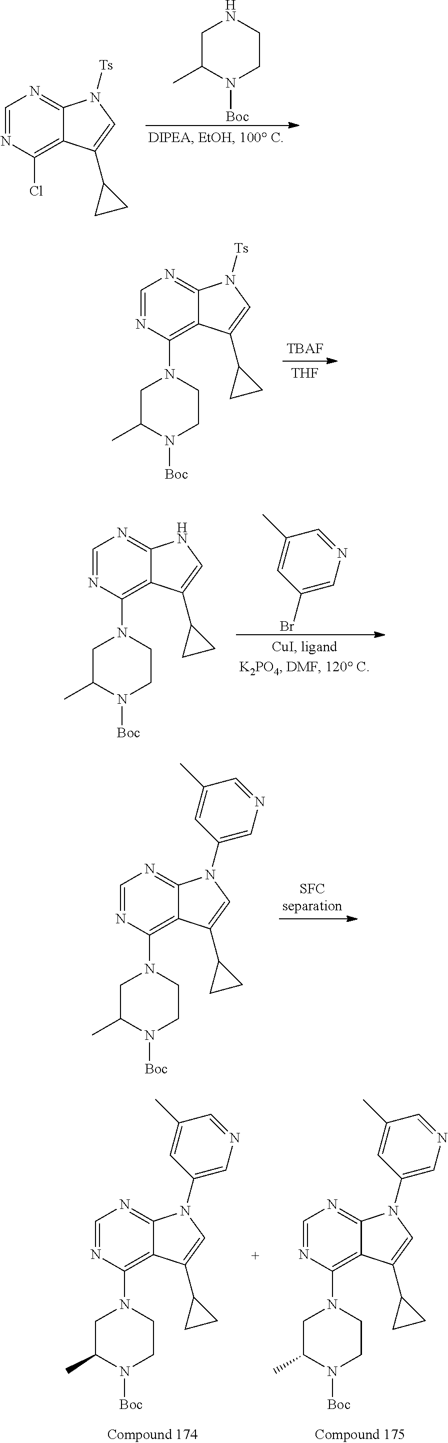

### Step 1. tert-Butyl 4-(5-cyclopropyl-7-tosyl-7H-pyrrolo[2,3-d]pyrimidin-4-yl)-2-methylpiperazine-1-carboxylate

A mixture of 4-chloro-5-cyclopropyl-7-tosyl-7H-pyrrolo[2,3-d]pyrimidine (350 mg, 1.0 mmol, prepared following the procedures described for compound 164, tert-butyl 2-methylpiperazine-1-carboxylate (240 mg, 1.2 mmol) and DIEA (0.50 mL, 3.0 mmol) in EtOH (10 mL) was stirred at 100° C. for 12 hours. The reaction mixture was concentrated. The residue was purified by column chromatography on silica gel (0-40% EtOAc in petroleum ether) to give the tert-butyl 4-(5-cyclopropyl-7-tosyl-7H-pyrrolo[2,3-d]pyrimidin-4-yl)-2-methylpiperazine-1-carboxylate (390 mg, 0.76 mmol, 76%) as a yellow solid. LC/MS ESI (m/z): 512 (M+H)⁺.

### Step 2. tert-Butyl 4-(5-cyclopropyl-7H-pyrrolo[2,3-d]pyrimidin-4-yl)-2-methylpiperazine-1-carboxylate

To a solution of tert-butyl 4-(5-cyclopropyl-7-tosyl-7H-pyrrolo[2,3-d]pyrimidin-4-yl)-2-methylpiperazine-1-carboxylate (390 mg, 0.76 mmol) in THE (20 mL) was added TBAF (4.6 mL, 1.0M in THF) dropwise. The resulting mixture was stirred at room temperature for 12 hours. The reaction mixture was extracted with EtOAc (100 mL×2). The combined organic layers were washed with brine, dried over Na₂SO₄ and concentrated. The residue was purified by column chromatography on silica gel (0˜80% EtOAc in petroleum ether) to give the tert-butyl 4-{5-cyclopropyl-7H-pyrrolo[2,3-d]pyrimidin-4-yl}-2-methylpiperazine-1-carboxylate (220 mg, 0.62 mmol, 81%) as a white solid. LC/MS ESI (m/z): 358 (M+H)⁺.

### Step 3. tert-Butyl (S)-4-(5-cyclopropyl-7-(5-methylpyridin-3-yl)-7H-pyrrolo[2,3-d]pyrimidin-4-yl)-2-methylpiperazine-1-carboxylate and tert-butyl (R)-4-(5-cyclopropyl-7-(5-methylpyridin-3-yl)-7H-pyrrolo[2,3-d]pyrimidin-4-yl)-2-methylpiperazine-1-carboxylate

A mixture of tert-butyl 4-{5-cyclopropyl-7H-pyrrolo[2,3-d]pyrimidin-4-yl}-2-methylpiperazine-1-carboxylate (220 mg, 0.62 mmol), 3-bromo-5-methylpyridine (210 mg, 1.2 mmol), copper (I) iodide (35 mg, 0.18 mmol), trans-1,2-diaminocyclohexane (21 mg, 0.18 mmol) and K₃PO₄ (391 mg, 1.8 mmol) in DMF (20 mL) was stirred at 120° C. for 16 hours. The reaction mixture was concentrated. The residue was purified by column chromatography on silica gel (0-30% EtOAc in petroleum ether) to give the tert-butyl 4-[5-cyclopropyl-7-(5-methylpyridin-3-yl)-7H-pyrrolo[2,3-d]pyrimidin-4-yl]-2-methylpiperazine-1-carboxylate (120 mg, 0.27 mmol, 44%), which was separated by SFC to afford the two isomers: Peak 1 (shorter retention time): Assigned as tert-butyl (S)-4-(5-cyclopropyl-7-(5-methylpyridin-3-yl)-7H-pyrrolo[2,3-d]pyrimidin-4-yl)-2-methylpiperazine-1-carboxylate (39 mg) as a light yellow solid. LC/MS ESI (m/z): 449 (M+H)⁺. ¹H NMR (400 MHz, CDCl₃) δ 8.65 (d, J=2.3 Hz, 1H), 8.44-8.40 (m, 2H), 7.93 (s, 1H), 6.95 (d, J=0.8 Hz, 1H), 4.53-4.47 (m, 1H), 4.41 (s, 1H), 4.11 (d, J=12.9 Hz, 1H), 3.97 (d, J=13.1 Hz, 1H), 3.44-3.32 (m, 2H), 3.09 (td, J=12.4, 3.4 Hz, 1H), 2.45 (s, 3H), 2.08-2.01 (m, 1H), 1.50 (s, 9H), 1.23 (d, J=6.8 Hz, 3H), 1.03 (dd, J=8.3, 3.9 Hz, 2H), 0.91-0.86 (m, 1H), 0.70-0.65 (m, 1H). Peak 2 (longer retention time): Assigned as tert-butyl (R)-4-(5-cyclopropyl-7-(5-methylpyridin-3-yl)-7H-pyrrolo[2,3-d]pyrimidin-4-yl)-2-methylpiperazine-1-carboxylate (44 mg) as a light yellow solid. LC/MS ESI (m/z): 449 (M+H)⁺. ¹H NMR (400 MHz, CDCl₃) δ 8.65 (d, J=2.2 Hz, 1H), 8.42 (d, J=5.8 Hz, 2H), 7.93 (s, 1H), 6.95 (d, J=0.8 Hz, 1H), 4.55-4.46 (m, 1H), 4.41 (s, 1H), 4.11 (d, J=13.0 Hz, 1H), 3.97 (d, J=13.2 Hz, 1H), 3.44-3.31 (m, 2H), 3.09 (td, J=12.4, 3.4 Hz, 1H), 2.45 (s, 3H), 2.09-1.97 (m, 1H), 1.50 (s, 9H), 1.23 (d, J=6.8 Hz, 3H), 1.07-1.02 (m, 2H), 0.91-0.86 (m, 1H), 0.70-0.65 (m, 1H).

SFC Preparative separation method: Instrument: Waters Thar 80 preparative SFC; Column: ChiralPak IC, 250×21.2 mm ID, 5 μm; Mobile phase: A for CO₂ and B for MeOH+0.1% NH₃·H₂O; Gradient: B 40%; Flow rate: 50 mL/min; Back pressure: 100 bar; Column temperature: 35° C.; Wavelength: 220 nm; Cycle-time: 5.0 min; Elution time: 4 h.

### Example 43. Synthesis of 1,1,1-trifluoro-2-methylpropan-2-yl(S)-3-methyl-4-(7-(1-methyl-1H-pyrazol-4-yl)-5-phenyl-7H-pyrrolo[2,3-d]pyrimidin-4-yl)piperazine-1-carboxylate (Compound 183)

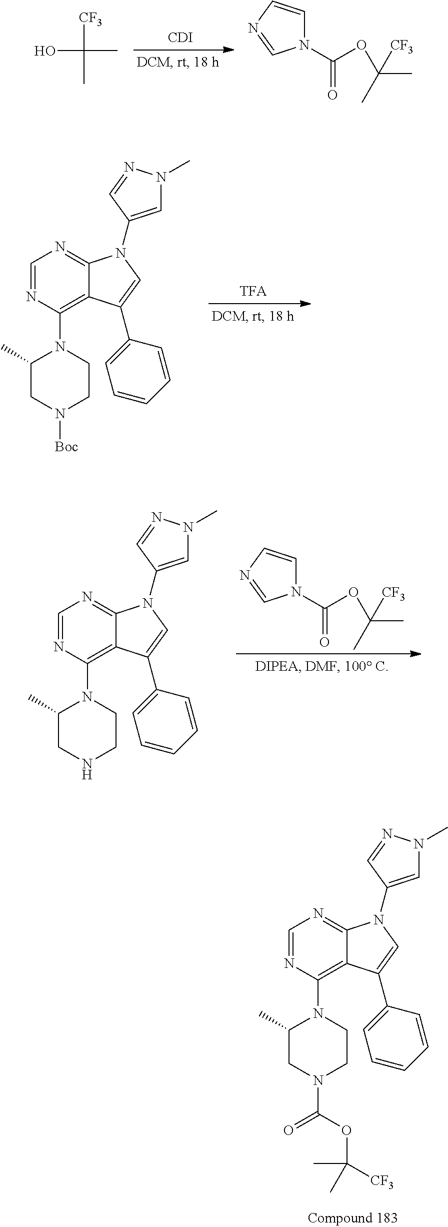

### Step 1. 1,1,1-Trifluoro-2-methylpropan-2-yl 1H-imidazole-1-carboxylate

To a solution of 1,1,1-trifluoro-2-methylpropan-2-ol (200 mg, 1.6 mmol) in DCM (5.0 mL) was added 1-(1H-imidazole-1-carbonyl)-1H-imidazole (250 mg, 1.6 mmol) at room temperature. The resulting mixture was stirred for 18 h. The reaction was quenched with water. The aqueous layer was extracted twice with DCM. The combined organic phases were washed with brine, dried over anhydrous Na₂SO₄, filtered and concentrated. The residue was used in the next step directly. LC/MS ESI (m/z): 223 (M+H)⁺

### Step 2. (S)-7-(1-Methyl-1H-pyrazol-4-yl)-4-(2-methylpiperazin-1-yl)-5-phenyl-7H-pyrrolo[2,3-d]pyrimidine

To a solution of tert-butyl(3S)-3-methyl-4-[7-(1-methyl-1H-pyrazol-4-yl)-5-phenyl-7H-pyrrolo[2,3-d]pyrimidin-4-yl]piperazine-1-carboxylate (120 mg, 0.25 mmol, prepared following an analogous procedure described for compound 268) in DCM (5 mL) was added TFA (3 mL). The reaction was stirred at room temperature for 18 h. The mixture was concentrated. The residue was neutralized by addition of saturated Na₂CO₃ solution and extracted twice with EtOAc. The combined organic phases were washed with brine, dried over anhydrous Na₂SO₄, filtered and concentrated. The residue was used directly without further purification. LC/MS ESI (m/z): 374 (M+H)⁺

### Step 3. 1,1,1-Trifluoro-2-methylpropan-2-yl(S)-3-methyl-4-(7-(I-methyl-1H-pyrazol-4-yl)-5-phenyl-7H-pyrrolo[2,3-d]pyrimidin-4-yl)piperazine-1-carboxylate

A solution of (2S)-2-methyl-1-[7-(1-methyl-1H-pyrazol-4-yl)-5-phenyl-7H-pyrrolo[2,3-d]pyrimidin-4-yl]piperazine (50 mg, 0.13 mmol), 1,1,1-trifluoro-2-methylpropan-2-yl-1H-imidazole-1-carboxylate (49 mg, 0.22 mmol) and DIEA (0.050 mL, 0.33 mmol) in DMF (3 mL) was stirred at 80° C. for 2 days, then the mixture was cooled to room temperature and concentrated. The residue was partitioned between water and EtOAc. The aqueous layer was extracted twice by EtOAc. The combined organic phases were washed with brine, dried over anhydrous Na₂SO₄, filtered and concentrated. The residue was purified by column chromatography on silica gel (EtOAc in petroleum ether, 1:100 to 1:5 v/v) and prep-HPLC to give 1,1,1-trifluoro-2-methylpropan-2-yl (S)-3-methyl-4-(7-(1-methyl-1H-pyrazol-4-yl)-5-phenyl-7H-pyrrolo[2,3-d]pyrimidin-4-yl)piperazine-1-carboxylate (5.3 mg, 7.5%) as a white solid. LC/MS ESI (m/z): 528 (M+H)^{+ 1}H NMR (400 MHz, CD₃OD) δ 8.40 (s, 1H), 8.25 (s, 1H), 7.96 (s, 1H), 7.58-7.52 (m, 3H), 7.48 (dd, J=14.1, 6.2 Hz, 2H), 7.38 (t, J=7.3 Hz, 1H), 4.14 (dd, J=6.5, 4.0 Hz, 1H), 3.98 (s, 3H), 3.86-3.68 (m, 1H), 3.56 (d, J=13.2 Hz, 1H), 3.41 (dd, J=23.2, 14.2 Hz, 1H), 3.18-3.10 (m, 1H), 2.85 (dd, J=48.9, 12.1 Hz, 2H), 1.64 (s, 6H), 0.91 (d, J=6.7 Hz, 3H).

### Example 44. Synthesis of tert-butyl 4-(1-(3-chlorophenyl)-3-(pyridin-2-yl)-1H-pyrrolo[3,2-c]pyridin-4-yl)-3-methylpiperazine-1-carboxylate (Compound 187)

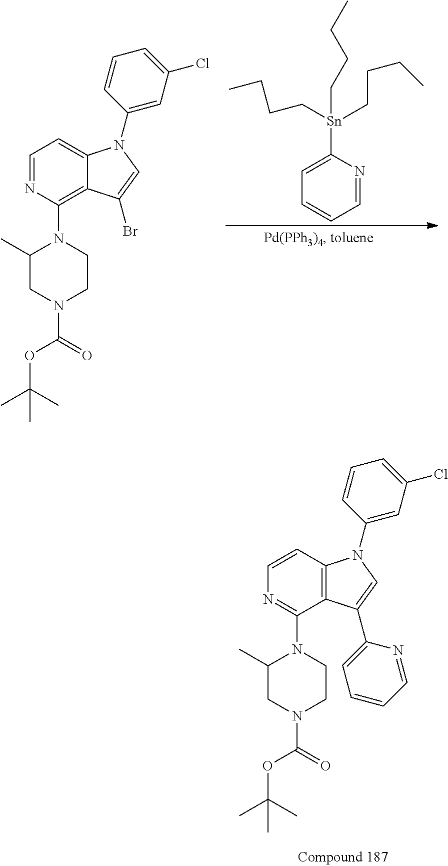

### Step 1. tert-Butyl 4-(1-(3-chlorophenyl)-3-(pyridin-2-yl)-1H-pyrrolo[3,2-c]pyridin-4-yl)-3-methylpiperazine-1-carboxylate

To a solution of tert-butyl 4-(3-bromo-1-(3-chlorophenyl)-1H-pyrrolo[3,2-c]pyridin-4-yl)-3-methylpiperazine-1-carboxylate (60 mg, 0.12 mmol, prepared following the procedures of the first two steps of compound 418 synthesis) in toluene (5 mL) were added 2-(tributylstannyl)pyridine (0.050 mL, 0.14 mmol) and Pd(PPh₃)₄ (14 mg, 0.012 mmol). The resulting mixture was stirred at 120° C. overnight. After cooling to room temperature, the reaction was quenched with KF (aq.) and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford tert-butyl 4-(1-(3-chlorophenyl)-3-(pyridin-2-yl)-1H-pyrrolo[3,2-c]pyridin-4-yl)-3-methylpiperazine-1-carboxylate (8.4 mg, 14%) as a white solid. LC/MS ESI (m/z): 504 (M+H)⁺. ¹H NMR (400 MHz, CD₃OD) δ 8.63 (d, J=4.7 Hz, 1H), 8.05 (d, J=6.0 Hz, 1H), 7.97 (td, J=7.7, 1.7 Hz, 1H), 7.85 (d, J=7.9 Hz, 1H), 7.78-7.75 (m, 1H), 7.69-7.67 (m, 1H), 7.64-7.55 (m, 2H), 7.51 (dt, J=7.5, 1.8 Hz, 1H), 7.47-7.42 (m, 1H), 7.26 (d, J=6.0 Hz, 1H), 3.56-3.42 (m, 2H), 3.24-3.07 (m, 4H), 2.88-2.73 (m, 1H), 1.43 (s, 9H), 0.84 (d, J=6.5 Hz, 3H).

The following compound was prepared by a procedure analogous to the synthesis of compound 187 using the corresponding tin reagent.

| Cmpd | | |
|---|---|---|
| No. | Chemical Name | LCMS and ¹H NMR |
| 190 | tert-Butyl 4-(1-(3-chlorophenyl)- | LC/MS ESI (m/z): 505 (M + H)⁺. ¹H NMR (400 MHz, |
| | 3-(pyrazin-2-yl)-1H-pyrrolo[3,2- | CD₃OD) δ 9.15 (s, 1H), 8.69 (d, J = 1.2 Hz, 1H), 8.57 (d, |
| | c]pyridin-4-yl)-3- | J = 2.3 Hz, 1H), 8.15-8.04 (m, 1H), 7.99-7.89 (m, 1H), |
| | methylpiperazine-1-carboxylate | 7.75-7.65 (m, 1H), 7.64-7.44 (m, 3H), 7.35-7.25 (m, |
| | | 1H), 3.53-3.42 (m, 1H), 3.29-2.89 (m, 6H), 1.44 (s, 9H), |
| | | 0.88 (d, J = 6.3 Hz, 3H). |

### Example 45. Synthesis of ethyl (3S,5S)-4-(7-(3-chlorophenyl)-5-cyclopropyl-7H-pyrrolo[2,3-d]pyrimidin-4-yl)-3,5-dimethylpiperazine-1-carboxylate (Compound 189)

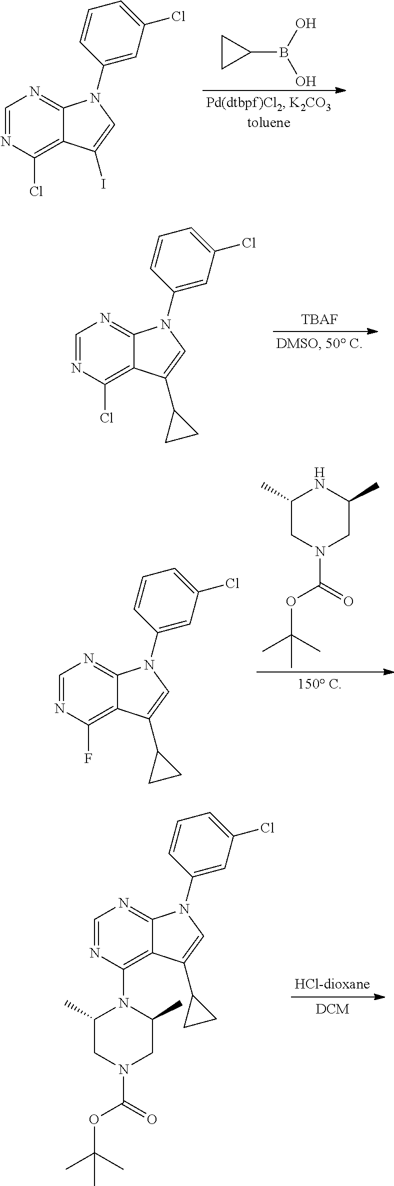

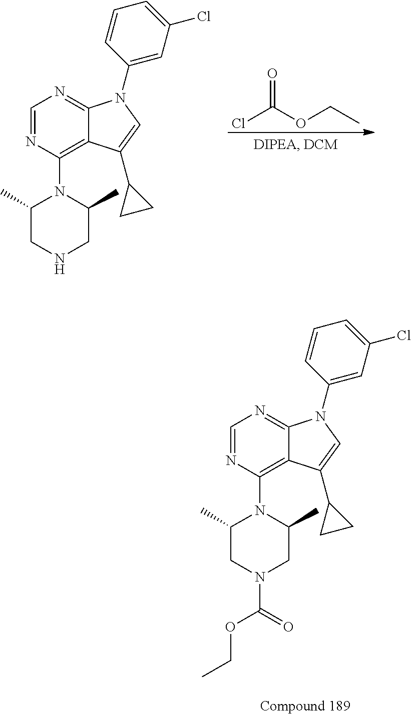

### Step 1. 4-Chloro-7-(3-chlorophenyl)-5-cyclopropyl-7H-pyrrolo[2,3-d]pyrimidine

A mixture of 4-chloro-7-(3-chlorophenyl)-5-iodo-7H-pyrrolo[2,3-d]pyrimidine (500 mg, 1.3 mmol, prepared following the procedures described for compound 274), cyclopropylboronic acid (110 mg, 1.3 mmol), Pd(dtbpf)Cl₂ (170 mg, 0.26 mmol) and K₂CO₃ (3.6 g, 26 mmol) in toluene (20 m1) was heated at 80° C. for 12 hours. The reaction mixture was concentrated. The residue was purified by column chromatography on silica gel (0˜40% EtOAc in petroleum ether) to give the 4-chloro-7-(3-chlorophenyl)-5-cyclopropyl-7H-pyrrolo[2,3-d]pyrimidine (300 mg, 77%) as a yellow solid. LC/MS ESI (m/z): 304 (M+H)⁺.

### Step 2. 7-(3-Chlorophenyl)-5-cyclopropyl-4-fluoro-7H-pyrrolo[2,3-d]pyrimidine

A mixture of 4-chloro-7-(3-chlorophenyl)-5-cyclopropyl-7H-pyrrolo[2,3-d]pyrimidine (300 mg, 1.0 mmol) and TBAF (2.0 ml, 1.0M in THF) in DMSO (20 mL) was stirred at 50° C. for 3 hours. The resulting mixture was quenched with ice water and extracted with EtOAc (50 mL×2). The combined organic layers were washed with brine, dried over Na₂SO₄ and concentrated. The residue was purified by column chromatography on silica gel (0˜30% EtOAc in petroleum ether) to give 7-(3-chlorophenyl)-5-cyclopropyl-4-fluoro-7H-pyrrolo[2,3-d]pyrimidine (120 mg, 42%) as a white solid. LC/MS ESI (m/z): 288 (M+H)⁺.

### Step 3. tert-Butyl (3S,5S)-4-(7-(3-chlorophenyl)-5-cyclopropyl-7H-pyrrolo[2,3-d]pyrimidin-4-yl)-3,5-dimethylpiperazine-1-carboxylate

A mixture of 7-(3-chlorophenyl)-5-cyclopropyl-4-fluoro-7H-pyrrolo[2,3-d]pyrimidine (100 mg, 0.35 mmol), tert-butyl (3S,5S)-3,5-dimethylpiperazine-1-carboxylate (150 mg, 0.70 mmol) and DIEA (0.17 mL, 1.1 mmol) in DMSO (10 mL) was stirred at 150° C. for 12 hours. After cooling to room temperature, the reaction mixture was partitioned, between EtOAc and water. The aqueous layer was extracted twice with EtOAc. The combined organic layers were washed with brine, dried over Na₂SO₄, filtered and concentrated. The residue was purified by flash column chromatography (silica gel, 0˜30% ethyl acetate in petroleum ether) to afford the tert-butyl (3S,5S)-4-(7-(3-chlorophenyl)-5-cyclopropyl-7H-pyrrolo[2,3-d]pyrimidin-4-yl)-3,5-dimethylpiperazine-1-carboxylate (60 mg, 35%). LC/MS ESI (m/z): 482 (M+H)⁺.

### Step 4. 7-(3-Chlorophenyl)-5-cyclopropyl-4-((2S,6S)-2,6-dimethylpiperazin-1-yl)-7H-pyrrolo[2,3-d]pyrimidine

To a solution of tert-butyl (3S,5S)-4-(7-(3-chlorophenyl)-5-cyclopropyl-7H-pyrrolo[2,3-d]pyrimidin-4-yl)-3,5-dimethylpiperazine-1-carboxylate (60 mg, 0.13 mmol) in DCM (10 mL) was added HCl (3.0 mL, 4.0M in dioxane). The resulting mixture was stirred at room temperature for 12 hours. The reaction mixture was concentrated to afford 7-(3-chlorophenyl)-5-cyclopropyl-4-((2S,6S)-2,6-dimethylpiperazin-1-yl)-7H-pyrrolo[2,3-d]pyrimidine (48 mg, 100%) as a yellow solid which was used in the next step directly without further purification. LC/MS ESI (m/z): 382 (M+H)⁺.

### Step 5. Ethyl (3S,5S)-4-(7-(3-chlorophenyl)-5-cyclopropyl-7H-pyrrolo[2,3-d]pyrimidin-4-yl)-3,5-dimethylpiperazine-1-carboxylate

To a mixture of 7-(3-chlorophenyl)-5-cyclopropyl-4-((2S,6S)-2,6-dimethylpiperazin-1-yl)-7H-pyrrolo[2,3-d]pyrimidine (40 mg, 0.11 mmol) and DIEA (0.050 mL, 0.31 mmol) in DCM (10 mL) at 0° C. was added ethyl carbonochloridate (14 mg, 0.13 mmol) dropwise. The resulting mixture was stirred at room temperature for 2 hours. The reaction mixture was quenched with NaHCO₃ (aq.), extracted with DCM (50 m1×2). The organic layers were washed with brine, dried over Na₂SO₄ and concentrated. The residue was purified by column chromatography on silica gel (0-50% EtOAc in petroleum ether) to give the crude product. The crude product was purified by HPLC to afford the ethyl (3S,5S)-4-(7-(3-chlorophenyl)-5-cyclopropyl-7H-pyrrolo[2,3-d]pyrimidin-4-yl)-3,5-dimethylpiperazine-1-carboxylate (15 mg, 0.033 mmol, 32%) 1006 as a white solid. LC/MS ESI (m/z): 454 (M+H)⁺. ¹H NMR (400 MHz, CD₃OD) δ 8.49 (s, 1H), 7.88 (t, J=2.0 Hz, 1H), 7.69-7.65 (m, 1H), 7.50 (t, J=8.1 Hz, 1H), 7.39-7.35 (m, 1H), 7.30 (s, 1H), 4.23-4.11 (m, 4H), 3.82 (s, 2H), 3.45 (s, 2H), 2.37-2.29 (m, 1H), 1.30 (t, J=5.4 Hz, 6H), 1.06 (d, J=6.3 Hz, 5H), 0.96-0.85 (m, 1H), 0.64-0.56 (m, 1H).

### Example 46. Synthesis of tert-butyl (R)-4-(7-(3-chlorophenyl)-5-(pyrrolidin-1-yl)-7H-pyrrolo[2,3-d]pyrimidin-4-yl)-2-methylpiperazine-1-carboxylate (Compound 193)

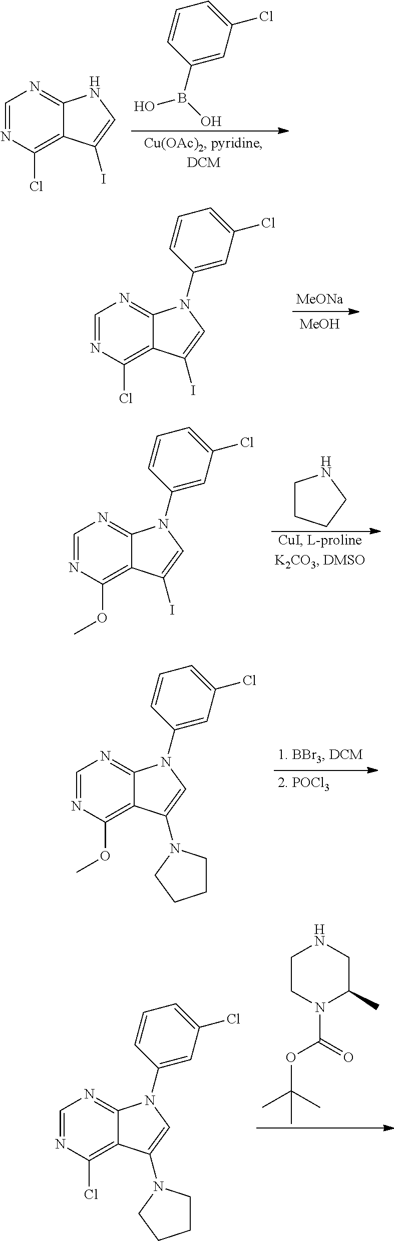

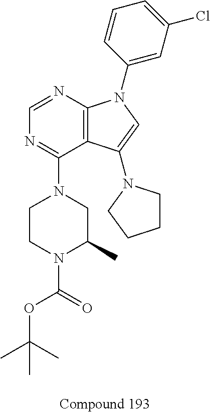 [SMILES: C[C@@H]1CN(c2ncnc3c2c(N2CCCC2)cn3-c2cccc(Cl)c2)CCN1C(=O)OC(C)(C)C]

### Step 1. 4-Chloro-7-(3-chlorophenyl)-5-iodo-7H-pyrrolo[2,3-d]pyrimidine

To a solution of 4-chloro-5-iodo-7H-pyrrolo[2,3-d]pyrimidine (10 g, 36 mmol) in DCM (600 mL) were added (3-chlorophenyl)boronic acid (11 g, 72 mmol), Cu(OAc)₂ (16 g, 110 mmol), pyridine (18 mL, 110 mmol) and 4 A molecular sieves (10 g). The resulting mixture was stirred at room temperature under an O₂ atmosphere for 3 days. After cooling with an ice-water bath, the reaction was quenched with NH₄OH (aq. solution, 50 mL) and filtered. The filtrate was extracted twice with DCM. The combined organic layers were dried over Na₂SO₄, filtered and concentrated. The residue was purified by flash chromatography (silica gel, 0˜30%, ethyl acetate in petroleum ether) to afford 4-chloro-7-(3-chlorophenyl)-5-iodo-7H-pyrrolo[2,3-d]pyrimidine (11 g, 79%) as a white solid. LC/MS ESI (m/z): 390 (M+H)⁺.

### Step 2. 7-(3-Chlorophenyl)-5-iodo-4-methoxy-7H-pyrrolo[2,3-d]pyrimidine

To a solution of 4-chloro-7-(3-chlorophenyl)-5-iodo-7H-pyrrolo[2,3-d]pyrimidine (4.0 g, 10 mmol) in MeOH (40 mL) was added MeONa (8.0 mL, 4.5M in MeOH). The resulting mixture was stirred at 50° C. overnight. The reaction was quenched with ice water, extracted twice with EtOAc. The combined organic layers were washed with brine, dried over Na₂SO₄, filtered and concentrated. The residue was purified by flash chromatography (silica gel, 0˜30%, ethyl acetate in petroleum ether) to afford 7-(3-chlorophenyl)-5-iodo-4-methoxy-7H-pyrrolo[2,3-d]pyrimidine (3.0 g, 79%) as a white solid. LC/MS ESI (m/z): 386 (M+H)⁺.

### Step 3. 7-(3-Chlorophenyl)-4-methoxy-5-(pyrrolidin-1-yl)-7H-pyrrolo[2,3-d]pyrimidine

To a solution of 7-(3-chlorophenyl)-5-iodo-4-methoxy-7H-pyrrolo[2,3-d]pyrimidine (2.3 g, 6 mmol) in DMSO (20 mL) were added CuI (230 mg, 1.2 mmol), K₂CO₃ (2.5 g, 18 mmol), L-proline (280 mg, 2.4 mmol) and pyrrolidine (850 mg, 12 mmol). The resulting mixture was heated at 90° C. overnight. After cooling to room temperature, the reaction mixture was partitioned between EtOAc and water. The aqueous layer was extracted twice with EtOAc. The combined organic layers were washed with brine, dried over Na₂SO₄, filtered and concentrated. The residue was purified by flash chromatography (silica gel, 0-40%, ethyl acetate in petroleum ether) to afford 7-(3-chlorophenyl)-4-methoxy-5-(pyrrolidin-1-yl)-7H-pyrrolo[2,3-d]pyrimidine (1.2 g, 61%) as a yellow solid. LC/MS ESI (m/z): 329 (M+H)⁺.

### Step 4. 7-(3-Chlorophenyl)-5-(pyrrolidin-1-yl)-7H-pyrrolo[2,3-d]pyrimidin-4-ol

A solution of 7-(3-chlorophenyl)-4-methoxy-5-(pyrrolidin-1-yl)-7H-pyrrolo[2,3-d]pyrimidine (0.50 g, 1.5 mmol) in DCM (10 mL) was treated with BBr₃ (3.0 mL) dropwise at 0° C. The resulting mixture was stirred at 0° C. for 2 h. The reaction was quenched with ice water and extracted twice with DCM. The combined organic layers were washed with brine, dried over Na₂SO₄, filtered and concentrated. The residue was purified by flash column chromatography (silica gel, 0˜50%, ethyl acetate in petroleum ether) to afford 7-(3-chlorophenyl)-5-(pyrrolidin-1-yl)-7H-pyrrolo[2,3-d]pyrimidin-4-ol (0.40 g, 84%) as a yellow solid. LC/MS ESI (m/z): 315 (M+H)⁺.

### Step 5. 4-Chloro-7-(3-chlorophenyl)-5-(pyrrolidin-1-yl)-7H-pyrrolo[2,3-d]pyrimidine

A solution of 7-(3-chlorophenyl)-5-(pyrrolidin-1-yl)-7H-pyrrolo[2,3-d]pyrimidin-4-ol (0.40 g, 1.2 mmol) in POCl₃ (4 mL) was heated at 120° C. overnight. After cooling to room temperature, the reaction was concentrated. The residue was re-dissolved in DCM and washed with NaHCO₃ (aq.). The aqueous layer was extracted twice with DCM. The combined organic layers were washed with brine, dried over Na₂SO₄, filtered and concentrated. The residue was purified by flash column chromatography (silica gel, 0-40%, ethyl acetate in petroleum ether) to afford 4-chloro-7-(3-chlorophenyl)-5-(pyrrolidin-1-yl)-7H-pyrrolo[2,3-d]pyrimidine (0.20 g, 48%) as a yellow solid. LC/MS ESI (m/z): 333 (M+H)⁺.

### Step 6. tert-Butyl (R)-4-(7-(3-chlorophenyl)-5-(pyrrolidin-1-yl)-7H-pyrrolo[2,3-d]pyrimidin-4-yl)-2-methylpiperazine-1-carboxylate

To a solution of 4-chloro-7-(3-chlorophenyl)-5-(pyrrolidin-1-yl)-7H-pyrrolo[2,3-d]pyrimidine (200 mg, 0.60 mmol) in EtOH (5 mL) were added DIPEA (230 mg, 1.8 mmol) and tert-butyl (R)-2-methylpiperazine-1-carboxylate (240 mg, 1.2 mmol). The resulting mixture was heated at 100° C. overnight. After cooling to room temperature, solvent was removed and the residue was purified by flash column chromatography (silica gel, 0˜40%, ethyl acetate in petroleum ether) to give crude product which was further purified by prep-HPLC to afford tert-butyl (R)-4-(7-(3-chlorophenyl)-5-(pyrrolidin-1-yl)-7H-pyrrolo[2,3-d]pyrimidin-4-yl)-2-methylpiperazine-1-carboxylate (36 mg, 12%) as a yellow solid. LC/MS ESI (m/z): 497 (M+H)⁺. ¹H NMR (400 MHz, CD₃OD) δ 8.22 (s, 1H), 7.85 (t, J=2.0 Hz, 1H), 7.67-7.63 (m, 1H), 7.49 (t, J=8.1 Hz, 1H), 7.34 (dd, J=8.0, 2.0 Hz, 1H), 7.03 (s, 1H), 4.84-4.78 (m, 2H), 4.50-4.45 (m, 1H), 4.37-4.31 (m, 1H), 3.92-3.86 (m, 1H), 3.40-3.33 (m, 1H), 3.28-3.25 (m, 2H), 3.00 (td, J=12.5, 3.6 Hz, 1H), 2.89-2.84 (m, 2H), 2.03-1.98 (m, 4H), 1.48 (s, 9H), 1.08 (d, J=6.8 Hz, 3H).

The following compounds were prepared by procedures analogous to the synthesis of compound 193 using the corresponding boronic acids and amines. In cases involving tert-butyl (S)-3-methylpiperazine-1-carboxylate or tert-butyl (R)-3-methylpiperazine-1-carboxylate in the last step, 5 eq. of the amine was used, and the reaction was run at 140° C.

| Cmpd | | |
|---|---|---|
| No. | Chemical Name | LCMS and ¹H NMR |
| 194 | tert-Butyl (S)-4-(7-(3- | LC/MS ESI (m/z): 497 (M + H)⁺. ¹H NMR (400 MHz, |
| | chlorophenyl)-5-(pyrrolidin-1- | CD₃OD) δ 8.22 (s, 1H), 7.85 (t, J = 2.0 Hz, 1H), 7.65 (dd, |
| | yl)-7H-pyrrolo[2,3-d]pyrimidin- | J = 8.1, 2.1 Hz, 1H), 7.49 (t, J = 8.1 Hz, 1H), 7.34 (dd, J = |
| | 4-yl)-2-methylpiperazine-1- | 8.0, 2.0 Hz, 1H), 7.03 (s, 1H), 4.84-4.78 (m, 2H), 4.50- |
| | carboxylate | 4.44 (m, 1H), 4.37-4.31 (m, 1H), 3.92-3.87 (m, 1H), |
| | | 3.40-3.33 (m, 1H), 3.28-3.25 (m, 2H), 3.04-2.97 (m, |
| | | 1H), 2.89-2.83 (m, 2H), 2.03-1.98 (m, 4H), 1.48 (s, 9H), |
| | | 1.08 (d, J = 6.8 Hz, 3H). |
| 209 | tert-Butyl (R)-4-(7-(3- | LC/MS ESI (m/z): 497 (M + H)⁺. ¹H NMR (400 MHz, |
| | chlorophenyl)-5-(pyrrolidin-1- | CD₃OD) δ 8.24 (s, 1H), 7.85 (t, J = 2.0 Hz, 1H), 7.66 (ddd, |
| | yl)-7H-pyrrolo[2,3-d]pyrimidin- | J = 8.1, 2.0, 0.9 Hz, 1H), 7.49 (t, J = 8.1 Hz, 1H), 7.35 |
| | 4-yl)-3-methylpiperazine-1- | (ddd, J = 8.1, 2.0, 0.9 Hz, 1H), 7.04 (s, 1H), 5.14-5.02 (m, |
| | carboxylate | 1H), 4.22-4.07 (m, 2H), 3.95-3.90 (m, 1H), 3.49-3.32 |
| | | (m, 2H), 3.24-3.18 (m, 2H), 3.13-3.02 (m, 1H), 2.97- |
| | | 2.90 (m, 2H), 2.02-1.98 (m, 4H), 1.49 (s, 9H), 1.09 (d, J = |
| | | 6.4 Hz, 3H). |
| 210 | tert-Butyl (S)-4-(7-(3- | LC/MS ESI (m/z): 497 (M + H)⁺. ¹H NMR (400 MHz, |
| | chlorophenyl)-5-(pyrrolidin-1- | CD₃OD) δ 8.24 (s, 1H), 7.85 (t, J = 2.0 Hz, 1H), 7.68-7.64 |
| | yl)-7H-pyrrolo[2,3-d]pyrimidin- | (m, 1H), 7.49 (t, J = 8.1 Hz, 1H), 7.35 (ddd, J = 8.1, 2.0, 0.9 |
| | 4-yl)-3-methylpiperazine-1- | Hz, 1H), 7.04 (s, 1H), 5.15-5.00 (m, 1H), 4.23-4.06 (m, |
| | carboxylate | 2H), 3.95-3.89 (m, 1H), 3.49-3.32 (m, 2H), 3.25-3.19 |
| | | (m, 2H), 3.14-3.02 (m, 1H), 2.96-2.89 (m, 2H), 2.03- |
| | | 1.97 (m, 4H), 1.49 (s, 9H), 1.10 (d, J = 6.5 Hz, 3H). |
| 227 | tert-Butyl (R)-4-(7-(3,5- | LC/MS ESI (m/z): 515 (M + H)⁺. ¹H NMR (400 MHz, |
| | difluorophenyl)-5-morpholino- | CD₃OD) δ 8.30 (s, 1H), 7.61-7.56 (m, 2H), 7.20 (s, 1H), |
| | 7H-pyrrolo[2,3-d]pyrimidin-4- | 6.93 (tt, J = 9.1, 2.3 Hz, 1H), 5.19-5.08 (m, 1H), 4.31- |
| | yl)-3-methylpiperazine-1- | 4.17 (m, 1H), 4.13-4.05 (m, 1H), 3.94-3.84 (m, 5H), |
| | carboxylate | 3.50-3.35 (m, 2H), 3.19-3.11 (m, 2H), 3.07-2.95 (m, |
| | | 1H), 2.93-2.85 (m, 2H), 1.50 (s, 9H), 1.12 (d, J = 6.6 Hz, |
| | | 3H). ¹⁹F NMR (377 MHz, CD₃OD) δ −110.49 (s). |
| 228 | tert-Butyl (S)-4-(7-(3,5- | LC/MS ESI (m/z): 515 (M + H)⁺. ¹H NMR (400 MHz, |
| | difluorophenyl)-5-morpholino- | CD₃OD) δ 8.30 (s, 1H), 7.62-7.56 (m, 2H), 7.20 (s, 1H), |
| | 7H-pyrrolo[2,3-d]pyrimidin-4- | 6.93 (tt, J = 9.0, 2.3 Hz, 1H), 5.18-5.09 (m, 1H), 4.31- |
| | yl)-3-methylpiperazine-1- | 4.18 (m, 1H), 4.13-4.06 (m, 1H), 3.95-3.84 (m, 5H), |
| | carboxylate | 3.50-3.37 (m, 2H), 3.20-3.11 (m, 2H), 3.09-2.94 (m, |
| | | 1H), 2.94-2.84 (m, 2H), 1.50 (s, 9H), 1.12 (d, J = 6.5 Hz, |
| | | 3H). |
| 229 | tert-Butyl (R)-4-(7-(3- | LC/MS ESI (m/z): 513 (M + H)⁺. ¹H NMR (400 MHz, |
| | chlorophenyl)-5-morpholino- | CD₃OD) δ 8.26 (s, 1H), 7.85 (t, J = 2.0 Hz, 1H), 7.66 (ddd, |
| | 7H-pyrrolo[2,3-d]pyrimidin-4- | J = 8.1, 2.1, 0.9 Hz, 1H), 7.50 (t, J = 8.1 Hz, 1H), 7.37 |
| | yl)-3-methylpiperazine-1- | (ddd, J = 8.1, 2.0, 0.9 Hz, 1H), 7.15 (s, 1H), 5.22-5.09 (m, |
| | carboxylate | 1H), 4.34-4.19 (m, 1H), 4.13-4.06 (m, 1H), 3.95-3.85 |
| | | (m, 5H), 3.49-3.33 (m, 2H), 3.20-3.11 (m, 2H), 3.07- |
| | | 2.95 (m, 1H), 2.94-2.85 (m, 2H), 1.50 (s, 9H), 1.12 (d, J = |
| | | 6.6 Hz, 3H). |
| 230 | tert-Butyl (S)-4-(7-(3- | LC/MS ESI (m/z): 513 (M + H)⁺. ¹H NMR (400 MHz, |
| | chlorophenyl)-5-morpholino- | CD₃OD) δ 8.26 (s, 1H), 7.85 (t, J = 2.0 Hz, 1H), 7.66 (ddd, |
| | 7H-pyrrolo[2,3-d]pyrimidin-4- | J = 8.1, 2.0, 0.9 Hz, 1H), 7.50 (t, J = 8.1 Hz, 1H), 7.37 |
| | yl)-3-methylpiperazine-1- | (ddd, J = 8.1, 2.0, 0.9 Hz, 1H), 7.15 (s, 1H), 5.22-5.10 (m, |
| | carboxylate | 1H), 4.34-4.18 (m, 1H), 4.14-4.05 (m, 1H), 3.95-3.84 |
| | | (m, 5H), 3.51-3.34 (m, 2H), 3.20-3.11 (m, 2H), 3.08- |
| | | 2.95 (m, 1H), 2.94-2.84 (m, 2H), 1.50 (s, 9H), 1.12 (d, J = |
| | | 6.5 Hz, 3H). |
| 358 | tert-Butyl (S)-4-(7-(3- | LC/MS ESI (m/z): 516 (M + H)⁺. ¹H NMR (400 MHz, |
| | cyanophenyl)-5-(3,3- | CD₃OD) δ 8.28-8.24 (m, 2H), 8.11-8.07 (m, 1H), 7.70- |
| | dimethylpyrrolidin-1-yl)-7H- | 7.66 (m, 2H), 7.08 (s, 1H), 5.22-5.02 (m, 1H), 4.34-4.02 |
| | pyrrolo[2,3-d]pyrimidin-4-yl)-3- | (m, 3H), 3.92 (d, J = 13.2 Hz, 1H), 3.49-3.39 (m, 2H), |
| | methylpiperazine-1-carboxylate | 3.11-2.99 (m, 1H), 2.98-2.89 (m, 2H), 2.83 (d, J = 8.9 |
| | | Hz, 1H), 2.04-2.01 (m, 1H), 1.64-1.57 (m, 1H), 1.49 (s, |
| | | 9H), 1.30 (br, 3H), 1.23 (d, J = 5.4 Hz, 6H). |

### Example 47. Synthesis of tert-butyl (R)-4-(5-cyclopropyl-7-(3,5-difluorophenyl)-7H-pyrrolo[2,3-d]pyrimidin-4-yl)-3-methylpiperazine-1-carboxylate (Compound 211)

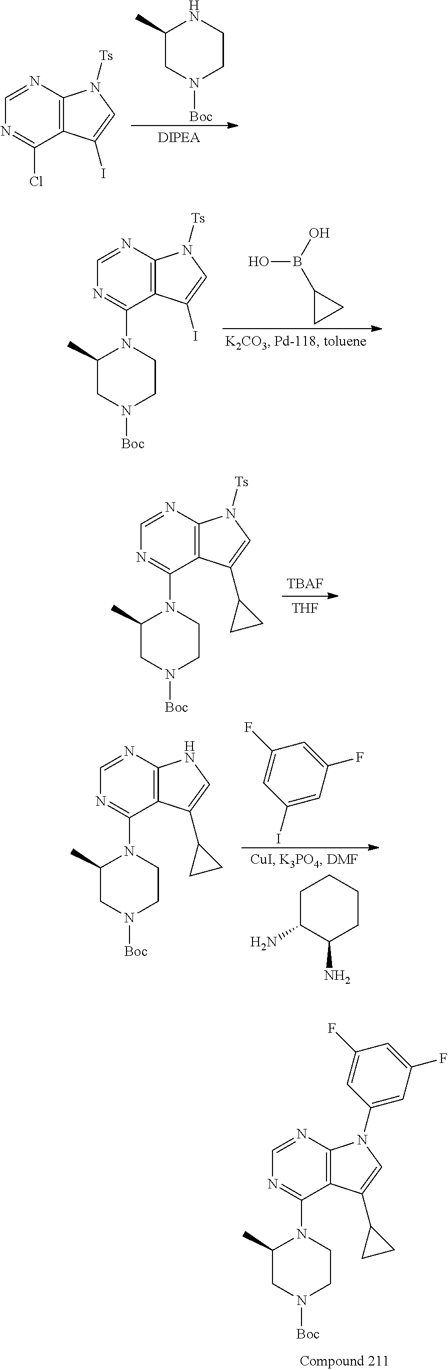

### Step 1. tert-Butyl (R)-4-(5-iodo-7-tosyl-7H-pyrrolo[2,3-d]pyrimidin-4-yl)-3-methylpiperazine-1-carboxylate

To a solution of 4-chloro-5-iodo-7-tosyl-7H-pyrrolo[2,3-d]pyrimidine (12 g, 28 mmol, prepared following step 1 of the procedures described for compound 192 in DIPEA (100 mL) was added tert-butyl (R)-3-methylpiperazine-1-carboxylate (11 g, 55 mmol). The resulting mixture was heated to 150° C. for 3 h. After cooling to room temperature, solvent was removed and the residue was purified by flash column chromatography (silica gel, 0˜30%, ethyl acetate in petroleum ether) to afford tert-butyl (R)-4-(5-iodo-7-tosyl-7H-pyrrolo[2,3-d]pyrimidin-4-yl)-3-methylpiperazine-1-carboxylate (11 g, 66%) as a white solid. LC/MS ESI (m/z): 598 (M+H)⁺.

### Step 2. tert-Butyl (R)-4-(5-cyclopropyl-7-tosyl-7H-pyrrolo[2,3-d]pyrimidin-4-yl)-3-methylpiperazine-1-carboxylate

To a solution of tert-butyl (R)-4-(5-iodo-7-tosyl-7H-pyrrolo[2,3-d]pyrimidin-4-yl)-3-methylpiperazine-1-carboxylate (3.5 g, 5.9 mmol) in toluene (30 mL) were added cyclopropylboronic acid (1.0 g, 12 mmol), K₂CO₃ (11 g, 77 mmol) and Pd-118 (390 mg, 0.59 mmol). The resulting mixture was heated to 80° C. overnight. After cooling to room temperature, the reaction mixture was filtered. The filtrate was concentrated and purified by flash column chromatography (silica gel, 0-30%, ethyl acetate in petroleum ether) to afford tert-butyl (R)-4-(5-cyclopropyl-7-tosyl-7H-pyrrolo[2,3-d]pyrimidin-4-yl)-3-methylpiperazine-1-carboxylate (1.9 g, 63%) as a white solid. LC/MS ESI (m/z): 512 (M+H)⁺.

### Step 3. tert-Butyl (R)-4-(5-cyclopropyl-7H-pyrrolo[2,3-d]pyrimidin-4-yl)-3-methylpiperazine-1-carboxylate

To a solution of tert-butyl (R)-4-(5-cyclopropyl-7-tosyl-7H-pyrrolo[2,3-d]pyrimidin-4-yl)-3-methylpiperazine-1-carboxylate (1.9 g, 3.7 mmol) in THE (30 mL) was added TBAF (22 mL, 1.0M in THF). The resulting mixture was stirred at room temperature overnight. The reaction was quenched with water and extracted twice with EtOAc. The combined organic layers were washed with brine, dried over Na₂SO₄, filtered and concentrated. The residue was purified by flash column chromatography (silica gel, 0-60%, ethyl acetate in petroleum ether) to afford tert-butyl (R)-4-(5-cyclopropyl-7H-pyrrolo[2,3-d]pyrimidin-4-yl)-3-methylpiperazine-1-carboxylate (1.1 g, 83%) as white solid. LC/MS ESI (m/z): 358 (M+H)⁺.

### Step 4. tert-Butyl (R)-4-(5-cyclopropyl-7-(3,5-difluorophenyl)-7H-pyrrolo[2,3-d]pyrimidin-4-yl)-3-methylpiperazine-1-carboxylate

To a solution of tert-butyl (R)-4-(5-cyclopropyl-7H-pyrrolo[2,3-d]pyrimidin-4-yl)-3-methylpiperazine-1-carboxylate (100 mg, 0.28 mmol) in DMF (10 mL) were added 1,3-difluoro-5-iodobenzene (130 mg, 0.56 mmol), trans-cyclohexane-1,2-diamine (9.7 mg, 0.084 mmol), CuI (27 mg, 0.15 mmol) and K₃PO₄ (180 mg, 0.84 mmol). The resulting mixture was heated to 120° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0-30%, ethyl acetate in petroleum ether) to give crude product which was further purified by prep-HPLC to afford tert-butyl (R)-4-(5-cyclopropyl-7-(3,5-difluorophenyl)-7H-pyrrolo[2,3-d]pyrimidin-4-yl)-3-methylpiperazine-1-carboxylate (44 mg, 33%) as a white solid. LC/MS ESI (m/z): 470 (M+H)⁺. ¹H NMR (400 MHz, CD₃OD) δ 8.33 (s, 1H), 7.57-7.51 (m, 2H), 7.29 (d, J=0.6 Hz, 1H), 6.97-6.89 (m, 1H), 4.75 (s, 1H), 4.06 (d, J=13.0 Hz, 1H), 3.86 (d, J=12.7 Hz, 2H), 3.59-3.34 (m, 2H), 3.24-3.07 (m, 1H), 2.10-2.02 (m, 1H), 1.50 (s, 9H), 1.20 (d, J=6.6 Hz, 3H), 1.07-1.01 (m, 2H), 0.91-0.86 (m, 1H), 0.80-0.72 (m, 1H).

The following compounds were prepared by procedures analogous to the synthesis of compound 211 from the corresponding aryl halides.

| Cmpd | | |
|---|---|---|
| No. | Chemical Name | LCMS and ¹H NMR |
| 195 | tert-butyl (R)-4-(5-cyclopropyl- | LC/MS ESI (m/z): 518 (M + H)⁺. ¹H NMR (400 MHz, |
| | 7-(3-(trifluoromethoxy)phenyl)- | CD₃OD) δ 8.32 (s, 1H), 7.79 (s, 1H), 7.71 (d, J = 9.4 Hz, |
| | 7H-pyrrolo[2,3-d]pyrimidin-4- | 1H), 7.60 (t, J = 8.2 Hz, 1H), 7.28 (d, J = 8.2 Hz, 2H), 4.80- |
| | yl)-3-methylpiperazine-1- | 4.75 (m, 1H), 4.07 (d, J = 13.9 Hz, 1H), 3.87 (d, J = 12.4 |
| | carboxylate | Hz, 2H), 3.59-3.38 (m, 2H), 3.23-3.12 (m, 1H), 2.09 (m, |
| | | 1H), 1.50 (s, 9H), 1.20 (d, J = 6.5 Hz, 3H), 1.07-1.02 (m, |
| | | 2H), 0.92-0.87 (m, 1H), 0.79-0.73 (m, 1H). |
| 197 | tert-butyl (R)-4-(5-cyclopropyl- | LC/MS ESI (m/z): 500 (M + H)⁺. ¹H NMR (400 MHz, |
| | 7-(3-(difluoromethoxy)phenyl)- | CD₃OD) δ 8.30 (d, J = 1.1 Hz, 1H), 7.59-7.41 (m, 3H), |
| | 7H-pyrrolo[2,3-d]pyrimidin-4- | 7.24 (s, 1H), 7.19-7.13 (m, 1H), 6.93 (t, J = 73.9 Hz, 1H), |
| | yl)-3-methylpiperazine-1- | 4.82-4.71 (m, 1H), 4.14-4.00 (m, 1H), 3.99-3.80 (m, |
| | carboxylate | 2H), 3.62-3.47 (m, 1H), 3.46-3.34 (m, 1H), 3.26-3.02 |
| | | (m, 1H), 2.14-2.04 (m, 1H), 1.50 (s, 9H), 1.20 (d, J = 6.5 |
| | | Hz, 3H), 1.08-1.00 (m, 2H), 0.92-0.84 (m, 1H), 0.79- |
| | | 0.70 (m, 1H). |
| 199 | tert-butyl (R)-4-(7-(3- | LC/MS ESI (m/z): 459 (M + H)⁺. ¹H NMR (400 MHz, |
| | cyanophenyl)-5-cyclopropyl-7H- | CD₃OD) δ 8.33 (s, 1H), 8.22-8.18 (m, 1H), 8.06 (dt, J = |
| | pyrrolo[2,3-d]pyrimidin-4-yl)-3- | 6.5, 2.5 Hz, 1H), 7.71-7.66 (m, 2H), 7.31 (d, J = 0.8 Hz, |
| | methylpiperazine-1-carboxylate | 1H), 4.78 (s, 1H), 4.07 (m, 1H), 3.88 (m, 2H), 3.60-3.34 |
| | | (m, 2H), 3.13 (m, 1H), 2.08 (m, 1H), 1.50 (s, 9H), 1.21 (d, |
| | | J = 6.6 Hz, 3H), 1.08-1.02 (m, 2H), 0.91-0.87 (m, 1H), |
| | | 0.80-0.73 (m, 1H). |
| 201 | tert-butyl (R)-4-(5-cyclopropyl- | LC/MS ESI (m/z): 464 (M + H)⁺. ¹H NMR (400 MHz, |
| | 7-(3-methoxyphenyl)-7H- | CD₃OD) δ 8.27 (s, 1H), 7.43-7.38 (m, 1H), 7.24 (t, J = 2.1 |
| | pyrrolo[2,3-d]pyrimidin-4-yl)-3- | Hz, 1H), 7.17 (d, J = 7.5 Hz, 2H), 6.97-6.92 (m, 1H), 4.78 |
| | methylpiperazine-1-carboxylate | (s, 1H), 4.07-4.04 (m, 1H), 3.87-3.71 (m, 5H), 3.61- |
| | | 3.36 (m, 2H), 3.16 (d, J = 25.2 Hz, 1H), 2.09 (s, 1H), 1.50 |
| | | (s, 9H), 1.20 (d, J = 6.5 Hz, 3H), 1.03 (d, J = 8.1 Hz, 2H), |
| | | 0.90-0.84 (m, 1H), 0.76-0.69 (m, 1H). |
| 393 | tert-butyl (R)-4-(7-(4- | LC/MS ESI (m/z): 460 (M + H)⁺. ¹H NMR (400 MHz, |
| | cyanopyridin-2-yl)-5- | CDCl3) δ 9.25 (s, 1H), 8.51 (d, J = 5.0 Hz, 1H), 8.43 (s, |
| | cyclopropyl-7H-pyrrolo [2,3- | 1H), 7.70 (s, 1H), 7.27 (d, J = 5.0 Hz, 1H), 4.63 (br. s, 1H), |
| | d]pyrimidin-4-yl)-3- | 4.11-3.73 (m, 3H), 3.48 (t, J = 11.2 Hz, 1H), 3.25 (m, |
| | methylpiperazine-1-carboxylate | 1H), 3.05 (m, 1H), 1.96 (m, 1H), 1.43 (s, 9H), 1.16 (d, J = |
| | | 6.5 Hz, 3H), 0.96 (d, J = 8.1 Hz, 2H), 0.79 (m, 1H), 0.70 |
| | | (m, 1H). |
| 394 | tert-butyl (2R,5S)-4-(7-(4- | LC/MS ESI (m/z): 474 (M + H)⁺. ¹H NMR (400 MHz, |
| | cyanopyridin-2-yl)-5- | CDCl3) δ 9.25 (s, 1H), 8.51 (d, J = 5.0 Hz, 1H), 8.42 (s, |
| | cyclopropyl-7H-pyrrolo[2,3- | 1H), 7.72 (s, 1H), 7.27 (dd, J = 5.0, 1.2 Hz, 1H), 4.85- |
| | d]pyrimidin-4-yl)-2,5- | 4.78 (m, 1H), 4.34 (br. s, 1H), 3.73-3.63 (m, 3H), 3.50 (d, |
| | dimethylpiperazine-1- | J = 11.7 Hz, 1H), 1.97-1.89 (m, 1H), 1.43 (s, 9H), 1.09 |
| | carboxylate | (dd, J = 6.6, 4.6 Hz, 6H), 0.97 (dd, J = 8.1, 2.0 Hz, 2H), |
| | | 0.90-0.84 (m, 1H), 0.65-0.59 (m, 1H). |
| 328 | tert-butyl (S)-4-(7-(3- | LC/MS ESI (m/z): 497 (M + H)⁺. ¹H NMR (400 MHz, |
| | cyanophenyl)-5-(pyrimidin-5- | CD₃OD) δ 9.18 (s, 1H), 9.09 (s, 2H), 8.53 (s, 1H), 8.36- |
| | yl)-7H-pyrrolo[2,3-d]pyrimidin- | 8.31 (m, 1H), 8.22-8.17 (m, 1H), 8.08 (s, 1H), 7.82-7.72 |
| | 4-yl)-3-methylpiperazine-1- | (m, 2H), 4.10-4.01 (m, 1H), 3.81 (d, J = 13.2 Hz, 1H), |
| | carboxylate | 3.64-3.41 (m, 2H), 3.26-3.14 (m, 1H), 2.93 (s, 2H), 1.43 |
| | | (s, 9H), 1.02 (d, J = 6.6 Hz, 3H). |
| 198 | tert-butyl (S)-4-(5-cyclopropyl- | LC/MS ESI (m/z): 500 (M + H)⁺. ¹H NMR (400 MHz, CDCl₃) |
| | 7-(3-(difluoromethoxy)phenyl)- | δ 8.44 (s, 1H), 7.55 (ddd, J = 8.0, 2.0, 1.0 Hz, 1H), 7.48 |
| | 7H-pyrrolo[2,3-d]pyrimidin-4- | (dd, J = 9.3, 6.8 Hz, 2H), 7.09 (d, J = 8.2 Hz, 1H), 6.92 (S, |
| | yl)-3-methylpiperazine-1- | 1H), 6.59 (t, J = 73.6 Hz, 1H), 4.74 (s, 1H), 4.14-3.82 (m, |
| | carboxylate | 3H), 3.55(t, J = 12.9 Hz, 1H), 3.38-3.10 (m, 2H), 2.07- |
| | | 2.01 (m, 1H), 1.50 (s, 9H), 1.24 (d, J = 6.6 Hz, 3H), 1.02 |
| | | (dd, J = 8.2, 1.9 Hz, 2H), 0.82-0.70 (m, 2H). |
| 202 | tert-butyl (S)-4-(5-cyclopropyl- | LC/MS ESI (m/z): 464 (M + H)⁺. ¹H NMR (400 MHz, |
| | 7-(3-methoxyphenyl)-7H- | CDCl₃) δ 8.44 (s, 1H), 7.41-7.37 (m, 1H), 7.20 (dd, J = |
| | pyrrolo[2,3-d]pyrimidin-4-yl)-3- | 7.2, 1.1 Hz, 2H), 6.93 (s, 1H), 6.90-6.86 (m, 1H), 4.73 (s, |
| | methylpiperazine-1-carboxylate | 1H), 4.18-3.89 (m, 2H), 3.86 (s, 3H), 3.85-3.75 (m, 1H), |
| | | 3.55 (t, J = 13.7Hz, 1H), 3.43-3.13 (m, 2H), 2.05 (t, J = |
| | | 7.5 Hz, 1H), 1.50 (s, 9H), 1.24 (d, J = 6.5 Hz, 3H), 1.01 |
| | | (dd, J = 8.2, 1.8 Hz, 2H), 0.81-0.68 (m, 2H). |
| 196 | tert-butyl (S)-4-(5-cyclopropyl- | LC/MS ESI (m/z): 518 (M + H)⁺. ¹H NMR (400 MHz, |
| | 7-(3-(trifluoromethoxy)phenyl)- | CDCl₃) δ 8.44 (s, 1H), 7.67 (ddd, J = 8.1, 2.0, 0.8 Hz, 1H), |
| | 7H-pyrrolo[2,3-d]pyrimidin-4- | 7.58 (s, 1H), 7.51 (t, J = 8.2 Hz, 1H), 7.21-7.17 (m, 1H), |
| | yl)-3-methylpiperazine-1- | 6.92 (d, J = 0.8 Hz, 1H), 4.74 (s, 1H), 4.14-3.80 (m, 3H), |
| | carboxylate | 3.55 (t, J = 13.1Hz, 1H), 3.39-3.27 (m, 1H), 3.21-3.07 |
| | | (m, 1H), 2.09-2.02 (m, 1H), 1.50 (s, 9H), 1.24 (d, J = 6.6 |
| | | Hz, 3H), 1.03 (dd, J = 8.2, 1.8 Hz, 2H), 0.82-0.69 (m, 2H). |
| 200 | tert-butyl (S)-4-(7-(3- | LC/MS ESI (m/z): 459 (M + H)⁺. ¹H NMR (400 MHz, |
| | cyanophenyl)-5-cyclopropyl-7H- | CDCl₃) δ 8.43 (s, 1H), 8.04 (d, J = 1.1 Hz, 1H), 8.01-7.97 |
| | pyrrolo[2,3-d]pyrimidin-4-yl)-3- | (m, 1H), 7.62-7.59 (m, 2H), 6.93 (d, J = 0.6 Hz, 1H), 4.75 |
| | methylpiperazine-1-carboxylate | (s, 1H), 4.15-3.84 (m, 3H), 3.53 (m, 1H), 3.24 (m, 2H), |
| | | 2.09-2.02 (m, 1H), 1.50 (s, 9H), 1.25 (d, J = 6.5 Hz, 3H), |
| | | 1.04 (dd, J = 8.2, 1.8 Hz, 2H), 0.77 (m, 2H). |
| 203 | tert-butyl (S)-4-(5-cyclopropyl- | LC/MS ESI (m/z): 470 (M + H)⁺. ¹H NMR (400 MHz, |
| | 7-(3,5-difluorophenyl)-7H- | CDCl₃) δ 8.45 (s, 1H), 7.35 (dd, J = 8.2, 2.1 Hz, 2H), 6.90 |
| | pyrrolo[2,3-d]pyrimidin-4-yl)-3- | (s, 1H), 6.76 (tt, J = 8.8, 2.3 Hz, 1H), 4.73 (s, 1H), 4.17- |
| | methylpiperazine-1-carboxylate | 3.83 (m, 3H), 3.55 (t, J = 12.8 Hz, 1H), 3.23 (m, 2H), 2.04 |
| | | (m, 1H), 1.50 (s, 9H), 1.24 (d, J = 6.6 Hz, 3H), 1.03 (dd, J = |
| | | 8.2, 1.8 Hz, 2H), 0.82-0.70 (m, 2H). |
| 219 | tert-butyl (S)-4-(5-cyclopropyl- | LC/MS ESI (m/z): 470 (M + H)⁺. ¹H NMR (400 MHz, |
| | 7-(3,4-difluorophenyl)-7H- | CDCl₃) δ 8.43 (s, 1H), 7.59 (ddd, J = 11.1, 7.0, 2.5 Hz, 1H), |
| | pyrrolo[2,3-d]pyrimidin-4-yl)-3- | 7.41-7.35 (m, 1H), 7.30-7.24 (m, 1H), 6.87 (s, 1H), 4.74 |
| | methylpiperazine-1-carboxylate | (s, 1H), 4.20-3.75 (m, 3H), 3.55 (dd, J = 14.0, 11.4 Hz, |
| | | 1H), 3.35 (m, 1H), 3.21-3.04 (m, 1H), 2.06-1.99 (m, |
| | | 1H), 1.50 (s, 9H), 1.24 (d, J = 6.6 Hz, 3H), 1.02 (dd, J = |
| | | 8.2, 1.9 Hz, 2H), 0.81-0.68 (m, 2H). |
| 217 | tert-butyl (S)-4-(5-cyclopropyl- | LC/MS ESI (m/z): 470 (M + H)⁺. ¹H NMR (400 MHz, |
| | 7-(2,3-difluorophenyl)-7H- | CDCl₃) δ 8.42 (s, 1H), 7.39 (ddd, J = 6.0, 4.4, 2.2 Hz, 1H), |
| | pyrrolo[2,3-d]pyrimidin-4-yl)-3- | 7.24-7.20 (m, 2H), 6.85 (d, J = 1.3 Hz, 1H), 4.78 (s, 1H), |
| | methylpiperazine-1-carboxylate | 4.20-3.55 (m, 3H), 3.56 (t, J = 11.1 Hz, 1H), 3.33 (m, |
| | | 1H), 3.13 (m, 1H), 2.03 (m, 1H), 1.50 (s, 9H), 1.26 (d, J = |
| | | 6.6 Hz, 3H), 1.01 (dd, J = 8.2, 1.8 Hz, 2H), 0.73 (m, 2H). |
| 218 | tert-butyl (S)-4-(5-cyclopropyl- | LC/MS ESI (m/z): 470 (M + H)⁺. ¹H NMR (400 MHz, |
| | 7-(2,5-difluorophenyl)-7H- | CDCl₃) δ 8.42 (s, 1H), 7.40 (ddd, J = 8.8, 5.9, 3.1 Hz, 1H), |
| | pyrrolo[2,3-d]pyrimidin-4-yl)-3- | 7.25-7.19 (m, 1H), 7.09-7.03 (m, 1H), 6.87-6.85 (m, |
| | methylpiperazine-1-carboxylate | 1H), 4.77 (s, 1H), 4.15-3.80 (m, 3H), 3.56 (t, J = 9.4 Hz, |
| | | 1H), 3.38-3.08 (m, 2H), 2.06-2.00 (m, 1H), 1.50 (s, 9H), |
| | | 1.25 (d, J = 6.5 Hz, 3H), 1.01 (dd, J = 8.2, 1.8 Hz, 2H), |
| | | 0.80-0.69 (m, 2H). |

### Example 48. Synthesis of tert-butyl (R)-4-(7-(3,5-difluorophenyl)-5-(pyridin-2-yl)-7H-pyrrolo[2,3-d]pyrimidin-4-yl)-2-methylpiperazine-1-carboxylate (Compound 215)

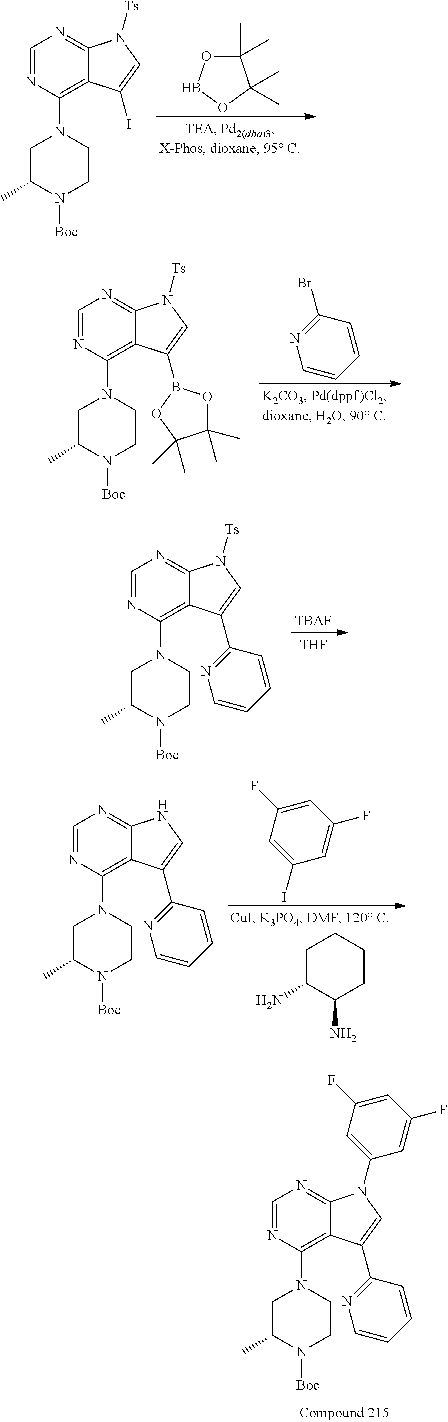

### Step 1. tert-Butyl (R)-2-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate

To a solution of tert-butyl (R)-4-(5-iodo-7-tosyl-7H-pyrrolo[2,3-d]pyrimidin-4-yl)-2-methylpiperazine-1-carboxylate (500 mg, 0.83 mmol, prepared following the procedures of the first two steps of compound 192 synthesis) in dioxane (10 mL) were added 4,4,5,5-tetramethyl-1,3,2-dioxaborolane (0.48 mL, 3.3 mmol), TEA (0.58 mL, 4.1 mmol), X-Phos (40 mg, 0.084 mmol) and Pd₂(dba)₃ (77 mg, 0.084 mmol). The resulting mixture was stirred at 95° C. overnight. After cooling to room temperature, the reaction was quenched with water and extracted twice with DCM. The combined organic layers were washed with brine, dried over Na₂SO₄, filtered and concentrated to afford crude tert-butyl (R)-2-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate as a brown oil, which was used in the next step directly. LC/MS ESI (m/z): 598 (M+H)⁺.

### Step 2. tert-Butyl (R)-2-methyl-4-(5-(pyridin-2-yl)-7-tosyl-7H-pyrrolo[2,3-d]pyrimidin-4-yl)piperazine-1-carboxylate

To a solution of tert-butyl (R)-2-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate (500 mg, 0.83 mmol) in dioxane (10 mL) and H₂O (2 mL) were added 2-bromopyridine (0.16 mL, 1.6 mmol), K₂CO₃ (580 mg, 4.1 mmol) and Pd(dppf)Cl₂ (61 mg, 0.084 mmol). The resulting mixture was heated at 90° C. overnight. After cooling to room temperature, solvent was removed and the residue was purified by flash column chromatography (silica gel, 0˜50%, ethyl acetate in petroleum ether) to afford tert-butyl (R)-2-methyl-4-(5-(pyridin-2-yl)-7-tosyl-7H-pyrrolo[2,3-d]pyrimidin-4-yl)piperazine-1-carboxylate (410 mg, 89%) as a yellow solid. LC/MS ESI (m/z): 549 (M+H)⁺.

### Step 3. tert-Butyl (R)-2-methyl-4-(5-(pyridin-2-yl)-7H-pyrrolo[2,3-d]pyrimidin-4-yl)piperazine-1-carboxylate

To a solution of tert-butyl (R)-2-methyl-4-(5-(pyridin-2-yl)-7-tosyl-7H-pyrrolo[2,3-d]pyrimidin-4-yl)piperazine-1-carboxylate (410 mg, 0.74 mmol) in THE (5 mL) was added TBAF (4.5 mL, 1.0M in THF). The resulting mixture was stirred at room temperature overnight. The reaction was quenched with water and extracted twice with EtOAc. The combined organic layers were washed with brine, dried over Na₂SO₄, filtered and concentrated. The residue was purified by flash column chromatography (silica gel, 0˜10%, methanol in dichloromethane) to afford tert-butyl (R)-2-methyl-4-(5-(pyridin-2-yl)-7H-pyrrolo[2,3-d]pyrimidin-4-yl)piperazine-1-carboxylate (240 mg, 81%) as a yellow solid. LC/MS ESI (m/z): 395 (M+H)⁺.

### Step 4. tert-Butyl (R)-4-(7-(3,5-difluorophenyl)-5-(pyridin-2-yl)-7H-pyrrolo[2,3-d]pyrimidin-4-yl)-2-methylpiperazine-1-carboxylate

To a solution of tert-butyl (R)-2-methyl-4-(5-(pyridin-2-yl)-7H-pyrrolo[2,3-d]pyrimidin-4-yl)piperazine-1-carboxylate (140 mg, 0.35 mmol) in DMF (5 mL) were added 1,3-difluoro-5-iodobenzene (100 mg, 0.42 mmol), trans-cyclohexane-1,2-diamine (12 mg, 0.11 mmol), CuI (20 mg, 0.11 mmol) and K₃PO₄ (230 mg, 1.1 mmol). The resulting mixture was heated at 120° C. overnight. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to give crude product which was further purified by prep-HPLC to afford tert-butyl (R)-4-(7-(3,5-difluorophenyl)-5-(pyridin-2-yl)-7H-pyrrolo[2,3-d]pyrimidin-4-yl)-2-methylpiperazine-1-carboxylate (110 mg, 61%) as a white solid. LC/MS ESI (m/z): 507 (M+H)⁺. ¹H NMR (400 MHz, CDCl₃) δ 8.72-8.67 (m, 1H), 8.52 (s, 1H), 7.79 (td, J=7.7, 1.8 Hz, 1H), 7.67 (s, 1H), 7.59 (d, J=7.8 Hz, 1H), 7.49-7.43 (m, 2H), 7.31-7.26 (m, 1H), 6.82 (tt, J=8.8, 2.3 Hz, 1H), 4.30-4.15 (m, 1H), 3.90-3.74 (m, 2H), 3.62-3.46 (m, 1H), 3.08 (dd, J=13.3, 3.9 Hz, 1H), 2.94-2.72 (m, 2H), 1.44 (s, 9H), 1.10 (d, J=6.8 Hz, 3H).

The following compounds were prepared by procedures analogous to the synthesis of compound 215 using the corresponding aryl halides in steps 2 and 4.

| Cmpd | | |
|---|---|---|
| No. | Chemical Name | LCMS and ¹H NMR |
| 221 | tert-butyl (R)-4-(7-(3-chloro-5- | LC/MS ESI (m/z): 523 (M + H)⁺. ¹H NMR (400 MHz, |
| | fluorophenyl)-5-(pyridin-2-yl)- | CDCl₃) δ 8.70 (d, J = 4.3 Hz, 1H), 8.52 (s, 1H), 7.79 (td, |
| | 7H-pyrrolo[2,3-d]pyrimidin-4- | J = 7.7, 1.7 Hz, 1H), 7.68-7.63 (m, 2H), 7.62-7.56 (m, |
| | yl)-2-methylpiperazine-1- | 2H), 7.31-7.26 (m, 1H), 7.10 (dt, J = 8.2, 2.0 Hz, 1H), |
| | carboxylate | 4.29-4.18 (m, 1H), 3.89-3.78 (m, 2H), 3.58-3.49 (m, |
| | | 1H), 3.08 (dd, J = 13.3, 3.9 Hz, 1H), 2.93-2.71 (m, 2H), |
| | | 1.44 (s, 9H), 1.10 (d, J = 6.8 Hz, 3H). |
| 223 | tert-butyl (R)-4-(7-(3-chloro-5- | LC/MS ESI (m/z): 524 (M + H)⁺. ¹H NMR (400 MHz, |
| | fluorophenyl)-5-(pyrazin-2-yl)- | CDCl₃) δ 8.91 (d, J = 0.9 Hz, 1H), 8.66-8.62 (m, 1H), |
| | 7H-pyrrolo[2,3-d]pyrimidin-4- | 8.56 (s, 1H), 8.55 (d, J = 2.4 Hz, 1H), 7.77 (s, 1H), 7.67- |
| | yl)-2-methylpiperazine-1- | 7.63 (m, 1H), 7.59 (dt, J = 9.4, 2.1 Hz, 1H), 7.14 (dt, J = |
| | carboxylate | 8.2, 2.1 Hz, 1H), 4.33-4.12 (m, 1H), 3.80 (d, J = 12.2 Hz, |
| | | 2H), 3.60 (d, J = 12.8 Hz, 1H), 3.15 (dd, J = 13.2, 3.9 Hz, |
| | | 1H), 3.00-2.79 (m, 2H), 1.44 (s, 9H), 1.12 (d, J = 6.8 Hz, 3H). |
| 233 | tert-butyl (R)-4-(7-(3,5- | LC/MS ESI (m/z): 508 (M + H)⁺. ¹H NMR (400 MHz, |
| | difluorophenyl)-5-(pyrazin-2- | CDCl₃) δ 8.91 (d, J = 1.5 Hz, 1H), 8.64 (dd, J = 2.5, 1.6 Hz, |
| | yl)-7H-pyrrolo[2,3-d]pyrimidin- | 1H), 8.56 (s, 1H), 8.55 (d, J = 2.5 Hz, 1H), 7.77 (s, 1H), |
| | 4-yl)-2-methylpiperazine-1- | 7.46 (dd, J = 7.9, 2.2 Hz, 2H), 6.86 (tt, J = 8.7, 2.3 Hz, 1H), |
| | carboxylate | 4.30-4.18 (m, 1H), 3.85-3.75 (m, 2H), 3.59 (d, J = 12.7 |
| | | Hz, 1H), 3.15 (dd, J = 13.2, 3.9 Hz, 1H), 3.01-2.80 (m, |
| | | 2H), 1.44 (s, 9H), 1.12 (d, J = 6.8 Hz, 3H). |
| 222 | tert-butyl (R)-4-(7-(3-chloro-5- | LC/MS ESI (m/z): 523 (M + H)⁺. ¹H NMR (400 MHz, |
| | fluorophenyl)-5-(pyridin-3-yl)- | CDCl₃) δ 8.83 (d, J = 1.8 Hz, 1H), 8.65 (dd, J = 4.8, 1.5 Hz, |
| | 7H-pyrrolo[2,3-d]pyrimidin-4- | 1H), 8.55 (s, 1H), 7.86 (dt, J = 7.8, 1.9 Hz, 1H), 7.64-7.61 |
| | yl)-2-methylpiperazine-1- | (m, 1H), 7.58 (dt, J = 9.5, 2.1 Hz, 1H), 7.42 (dd, J = 7.5, 4.9 |
| | carboxylate | Hz, 1H), 7.38 (s, 1H), 7.13 (dt, J = 8.2, 2.0 Hz, 1H), 4.32- |
| | | 4.20 (m, 1H), 3.81-3.72 (m, 1H), 3.69-3.60 (m, 1H), |
| | | 3.52-3.43 (m, 1H), 3.05 (dd, J = 13.1, 4.0 Hz, 1H), 2.79- |
| | | 2.66 (m, 2H), 1.43 (s, 9H), 1.13 (d, J = 6.8 Hz, 3H). |
| 216 | tert-butyl (R)-4-(7-(3,5- | LC/MS ESI (m/z): 507 (M + H)⁺. ¹H NMR (400 MHz, |
| | difluorophenyl)-5-(pyridin-4-yl)- | CDCl₃) δ 8.70 (dd, J = 4.5, 1.6 Hz, 2H), 8.55 (s, 1H), 7.48 |
| | 7H-pyrrolo[2,3-d]pyrimidin-4- | (dd, J = 4.5, 1.6 Hz, 2H), 7.45-7.40 (m, 3H), 6.85 (tt, J = |
| | yl)-2-methylpiperazine-1- | 8.7, 2.3 Hz, 1H), 4.33-4.18 (m, 1H), 3.75 (dd, J = 15.2, |
| | carboxylate | 13.1 Hz, 2H), 3.54 (d, J = 10.7 Hz, 1H), 3.09 (dd, J = 13.2, |
| | | 3.9 Hz, 1H), 2.90-2.72 (m, 2H), 1.43 (s, 9H), 1.10 (d, J = |
| | | 6.8 Hz, 3H). |
| 238 | tert-butyl (R)-4-(7-(3-chloro-5- | LC/MS ESI (m/z): 523 (M + H)⁺. ¹H NMR (400 MHz, |
| | fluorophenyl)-5-(pyridin-4-yl)- | CDCl₃) δ 8.70 (dd, J = 4.6, 1.4 Hz, 2H), 8.55 (s, 1H), 7.63- |
| | 7H-pyrrolo[2,3-d]pyrimidin-4- | 7.60 (m, 1H), 7.57 (dt, J = 9.4, 2.1 Hz, 1H), 7.47 (dd, J = |
| | yl)-2-methylpiperazine-1- | 4.5, 1.6 Hz, 2H), 7.43 (s, 1H), 7.13 (dt, J = 8.2, 2.1 Hz, 1H), |
| | carboxylate | 4.29-4.18 (m, 1H), 3.81-3.69 (m, 2H), 3.54 (d, J = 11.1 |
| | | Hz, 1H), 3.09 (dd, J = 13.2, 3.9 Hz, 1H), 2.88-2.75 (m, |
| | | 2H), 1.43 (s, 9H), 1.10 (d, J = 6.8 Hz, 3H). |
| 214 | tert-butyl (R)-4-(7-(3,5- | LC/MS ESI (m/z): 507 (M + H)⁺. ¹H NMR (400 MHz, |
| | difluorophenyl)-5-(pyridin-3-yl)- | CDCl₃) δ 8.86-8.81 (m, 1H), 8.65 (dd, J = 4.8, 1.6 Hz, |
| | 7H-pyrrolo[2,3-d]pyrimidin-4- | 1H), 8.56 (s, 1H), 7.89-7.83 (m, 1H), 7.46-7.40 (m, 3H), |
| | yl)-2-methylpiperazine-1- | 7.38 (s, 1H), 6.84 (tt, J = 8.8, 2.3 Hz, 1H), 4.32-4.17 (m, |
| | carboxylate | 1H), 3.80-3.72 (m, 1H), 3.67-3.60 (m, 1H), 3.51-3.43 |
| | | (m, 1H), 3.05 (dd, J = 13.1, 3.9 Hz, 1H), 2.80-2.65 (m, |
| | | 2H), 1.43 (s, 9H), 1.13 (d, J = 6.8 Hz, 3H). |

### Example 49. Synthesis of tert-butyl (2R,5S)-4-(5-cyclopropyl-7-(3,5-difluorophenyl)-7H-pyrrolo[2,3-d]pyrimidin-4-yl)-2,5-dimethylpiperazine-1-carboxylate (Compound 232)

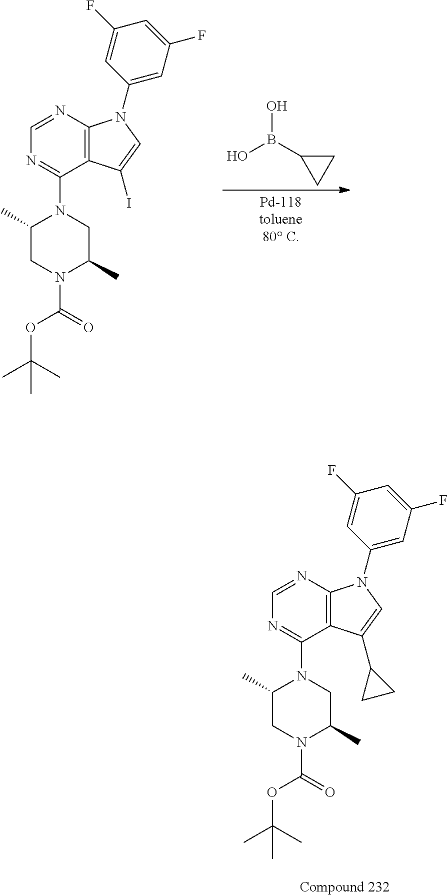 [SMILES: C[C@@H]1CN(c2ncnc3c2c(I)cn3-c2cc(F)cc(F)c2)[C@@H](C)CN1C(=O)OC(C)(C)C ; OB(O)C1CC1 ; C[C@@H]1CN(c2ncnc3c2c(C2CC2)cn3-c2cc(F)cc(F)c2)[C@@H](C)CN1C(=O)OC(C)(C)C]

To a solution of tert-butyl (2R,5S)-4-[7-(3,5-difluorophenyl)-5-iodo-7H-pyrrolo[2,3-d]pyrimidin-4-yl]-2,5-dimethylpiperazine-1-carboxylate (150 mg, 0.26 mmol, prepared following an analogous procedure described for compound 242) in toluene (5 mL) were added cyclopropylboronic acid (45 mg, 0.52 mmol), 1,1′-bis(di-t-butylphosphino)ferrocene palladium dichloride (17 mg, 0.030 mmol) and K₂CO₃ (730 mg, 5.3 mmol). The resulting mixture was heated to 80° C. overnight. After cooling to room temperature, solvent was removed and the residue was purified by flash column chromatography to afford tert-butyl (2R,5S)-4-(5-cyclopropyl-7-(3,5-difluorophenyl)-7H-pyrrolo[2,3-d]pyrimidin-4-yl)-2,5-dimethylpiperazine-1-carboxylate (90 mg, 71%) as a solid. LC/MS ESI (m/z): 484 (M+H)⁺. ¹H NMR (400 MHz, CDCl₃) δ 8.44 (s, 1H), 7.36 (dd, J=8.3, 2.2 Hz, 2H), 6.93 (d, J=0.8 Hz, 1H), 6.76 (tt, J=8.8, 2.3 Hz, 1H), 4.95-4.86 (m, 1H), 4.41 (s, 1H), 3.77 (m, 3H), 3.58 (d, J=11.0 Hz, 1H), 2.05-1.97 (m, 1H), 1.50 (s, 9H), 1.17 (t, J=6.8 Hz, 6H), 1.06-1.01 (m, 2H), 0.89-0.82 (m, 1H), 0.66 (dd, J=8.7, 4.3 Hz, 1H).

### Example 50. Synthesis of tert-butyl-(S)-4-(7-(3,5-difluorophenyl)-5-(pyridin-2-yl)-7H-pyrrolo[2,3-d]pyrimidin-4-yl)-3-methylpiperazine-1-carboxylate (Compound 235)

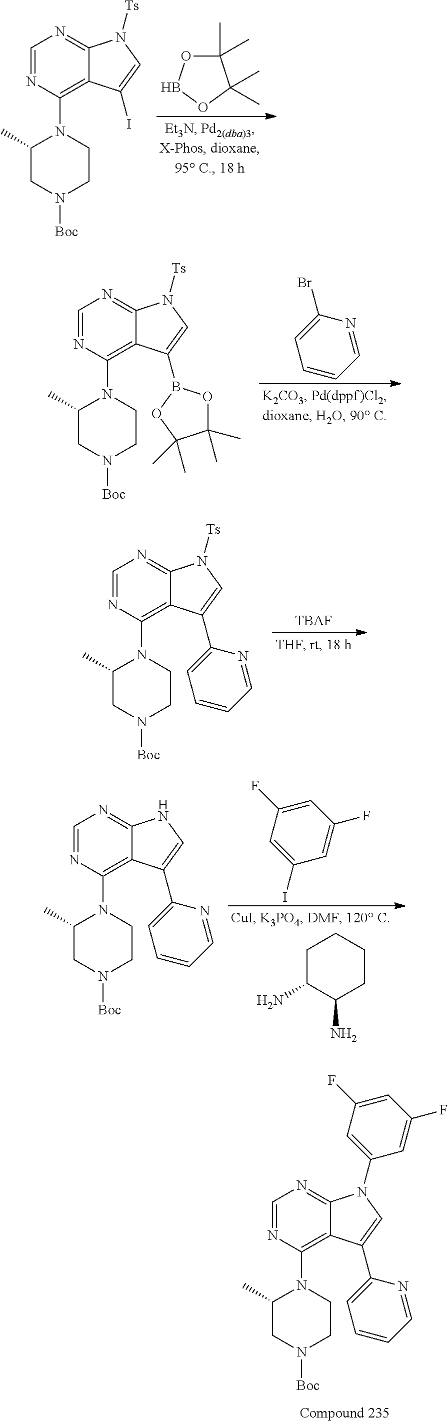

### Step 1. tert-Butyl-(S)-3-methyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate

A suspension of tert-butyl-(3S)-4-[5-iodo-7-(4-methylbenzenesulfonyl)-7H-pyrrolo[2,3-d]pyrimidin-4-yl]-3-methylpiperazine-1-carboxylate (1.8 g, 3.0 mmol, prepared following the procedure described for compound 268), 4,4,5,5-tetramethyl-1,3,2-dioxaborolane (1.3 mL, 9.0 mmol), Et₃N (1.6 mL, 12 mmol), X-Phos (0.43 g, 0.90 mmol) and Pd₂(dba)₃ (0.83 g, 0.90 mmol) in dioxane (30 mL) was stirred at 95° C. under a N₂ atmosphere for 18 h. The mixture was cooled to room temperature and concentrated. The residue was partitioned between water and EtOAc. The aqueous layer was extracted twice with EtOAc. The combined organic phases were washed with brine, dried over anhydrous Na₂SO₄, filtered and concentrated. The residue was used in the next step directly. LC/MS (ESI) (m/z): 598 (M+H)⁺

### Step 2. tert-Butyl-(S)-3-methyl-4-(5-(pyridin-2-yl)-7-tosyl-7H-pyrrolo[2,3-d]pyrimidin-4-yl)piperazine-1-carboxylate

A suspension of tert-butyl-(3S)-3-methyl-4-[7-(4-methylbenzenesulfonyl)-5-(tetramethyl-1,3,2-dioxaborolan-2-yl)-7H-pyrrolo[2,3-d]pyrimidin-4-yl]piperazine-1-carboxylate (500 mg, 0.84 mmol), 2-bromopyridine (130 mg, 0.84 mmol), K₂CO₃ (290 mg, 2.1 mmol) and Pd(dppf)Cl₂ (61 mg, 0.080 mmol) in dioxane-water (12 mL, 5:1 v/v) was stirred at 90° C. under a N₂ atmosphere for 18 h. After cooling to room temperature, solvent was removed, the residue was purified by column chromatography on silica gel (0˜10% MeOH in DCM) to give tert-butyl-(3S)-3-methyl-4-[7-(4-methylbenzenesulfonyl)-5-(pyridin-2-yl)-7H-pyrrolo[2,3-d]pyrimidin-4-yl]piperazine-1-carboxylate (200 mg, 44%) as a white solid. LC/MS (ESI) (m/z): 549 (M+H)⁺

### Step 3. tert-Butyl-(S)-3-methyl-4-(5-(pyridin-2-yl)-7H-pyrrolo[2,3-d]pyrimidin-4-yl)piperazine-1-carboxylate

To a solution of tert-butyl-(3S)-3-methyl-4-[7-(4-methylbenzenesulfonyl)-5-(pyridin-2-yl)-7H-pyrrolo[2,3-d]pyrimidin-4-yl]piperazine-1-carboxylate (200 mg, 0.36 mmol) in THE (5 mL) was added 1.0M TBAF (1.1 mL, 1.1 mmol). The resulting mixture was stirred at room temperature for 18 h. The reaction was partitioned between EtOAc and water. The aqueous layer was extracted twice with EtOAc. The combined organic phases were dried over anhydrous Na₂SO₄, filtered and concentrated. The residue was purified by column chromatography on silica gel (0˜20% MeOH in DCM) to give tert-butyl-(S)-3-methyl-4-(5-(pyridin-2-yl)-7H-pyrrolo[2,3-d]pyrimidin-4-yl)piperazine-1-carboxylate (120 mg, 83%) as a white solid. LC/MS (ESI) (m/z): 395 (M+H)⁺

### Step 4. tert-Butyl-(S)-4-(7-(3,5-difluorophenyl)-5-(pyridin-2-yl)-7H-pyrrolo[2,3-d]pyrimidin-4-yl)-3-methylpiperazine-1-carboxylate

A suspension of tert-butyl-(S)-3-methyl-4-(5-(pyridin-2-yl)-7H-pyrrolo[2,3-d]pyrimidin-4-yl)piperazine-1-carboxylate (80 mg, 0.20 mmol), 1,3-difluoro-5-iodobenzene (49 mg, 0.20 mmol), K₃PO₄ (110 mg, 0.51 mmol), CuI (12 mg, 0.060 mmol) and trans-cyclohexane-1,2-diamine (7.0 mg, 0.060 mmol) in DMF (5.0 mL) was stirred at 120° C. under a N₂ atmosphere for 18 h. The mixture was cooled to room temperature and concentrated. The residue was purified by column chromatography on silica gel (0˜10% MeOH in DCM) and prep-HPLC to give tert-butyl-(S)-4-(7-(3,5-difluorophenyl)-5-(pyridin-2-yl)-7H-pyrrolo[2,3-d]pyrimidin-4-yl)-3-methylpiperazine-1-carboxylate (55 mg, 54%) as a white solid. LC/MS(ESI) m/z: 507 (M+H)^{+ 1}H NMR (400 MHz, CDCl₃) δ 8.69 (dd, J=4.8, 0.7 Hz, 1H), 8.53 (s, 1H), 7.79 (td, J=7.7, 1.7 Hz, 1H), 7.68 (s, 1H), 7.59 (d, J=7.7 Hz, 1H), 7.50-7.41 (in 2H), 7.29-7.25 (m, 1H), 6.82 (tt, J=8.7, 2.3 Hz, 1H), 4.41-4.18 (m, 1H), 3.96-3.77 (m, 1H), 3.57 (m, 2H), 3.15 (t, J=11.7 Hz, 1H), 3.06-2.61 (m, 2H), 1.43 (s, 9H), 1.04 (s, 3H).

The following compounds were prepared by procedures analogous to the synthesis of compound 235 using the corresponding aryl halides.

| Cmpd | | |
|---|---|---|
| No. | Chemical Name | LCMS and ¹H NMR |
| 237 | tert-butyl-(S)-4-(7-(3,5- | LC/MS ESI (m/z): 508 (M + H)⁺. ¹H NMR (400 MHz, |
| | difluorophenyl)-5-(pyrazin-2- | CDCl₃) δ 8.92 (s, 1H), 8.58 (m, 3H), 7.79 (s, 1H), 7.50- |
| | yl)-7H-pyrrolo[2,3-d]pyrimidin- | 7.39 (m, 2H), 6.87 (tt, J = 8.7, 2.2 Hz, 1H), 4.24 (m, 1H), |
| | 4-yl)-3-methylpiperazine-1- | 3.90 (m, 1H), 3.69-3.42 (m, 2H), 3.29-2.72 (m, 3H), |
| | carboxylate | 1.44 (s, 9H), 1.09 (br. s, 3H). |
| 241 | tert-butyl-(S)-4-(7-(3,5- | LC/MS ESI (m/z): 507 (M + H)⁺. ¹H NMR (400 MHz, |
| | difluorophenyl)-5-(pyridin-4-yl)- | CDCl₃) δ 8.70 (s, 2H), 8.56 (s, 1H), 7.44 (m, 5H), 6.86 (tt, |
| | 7H-pyrrolo[2,3-d]pyrimidin-4- | J = 8.7, 2.2 Hz, 1H), 4.22 (m, 1H), 4.01-3.39 (m, 3H), 3.16 |
| | yl)-3-methylpiperazine-1- | (t, J = 11.8 Hz, 1H), 3.03-2.70 (m, 2H), 1.43 (s, 9H), 1.05 |
| | carboxylate | (d, J = 5.6 Hz, 3H). |
| 458 | tert-butyl (2R,5S)-4-(7-(4- | LC/MS ESI (m/z): 526 (M + H)⁺. ¹H NMR (400 MHz, |
| | cyanopyridin-2-yl)-5-(3- | CDCl₃) δ 9.43 (s, 1H), 8. 64-8.57 (m, 2H), 8.53 (s, 2H), |
| | methylpyrazin-2-yl)-7H- | 8.37 (s, 1H), 7.43 (d, J = 4.9 Hz, 1H), 4.21-3.75 (m, 2H), |
| | pyrrolo[2,3-d]pyrimidin-4-yl)- | 3.38-3.23 (m, 2H) 3.08 (s, 1H), 2.79 (m, 1H), 2.55 (s, |
| | 2,5-dimethylpiperazine-1- | 3H), 1.43 (s, 9H), 0.98 (m, 6H). |
| | carboxylate | |

### Example 51. Synthesis of tert-butyl (2R,5S)-4-(7-(3-chloro-5-fluorophenyl)-5-cyclopropyl-7H-pyrrolo[2,3-d]pyrimidin-4-yl)-2,5-dimethylpiperazine-1-carboxylate (Compound 236)

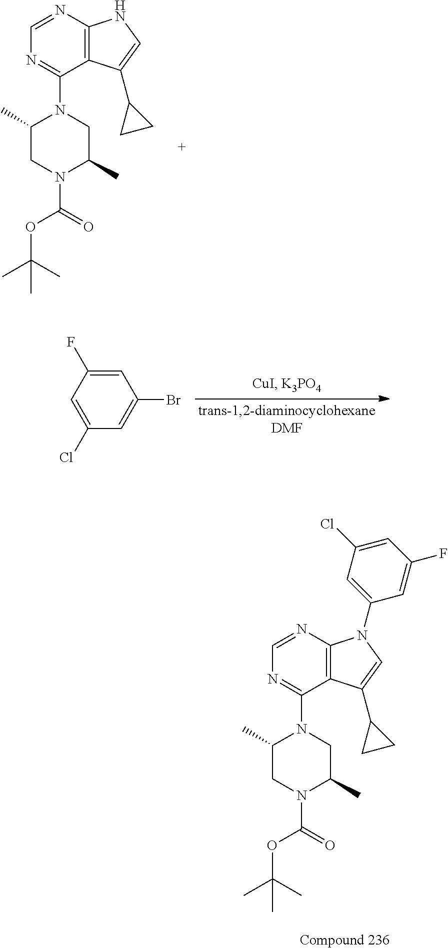 [SMILES: C[C@@H]1CN(c2ncnc3[nH]cc(C4CC4)c23)[C@@H](C)CN1C(=O)OC(C)(C)C ; Fc1cc(Cl)cc(Br)c1 ; C[C@@H]1CN(c2ncnc3c2c(C2CC2)cn3-c2cc(F)cc(Cl)c2)[C@@H](C)CN1C(=O)OC(C)(C)C]

To a solution of tert-butyl (2R,5S)-4-{5-cyclopropyl-7H-pyrrolo[2,3-d]pyrimidin-4-yl}-2,5-dimethylpiperazine-1-carboxylate (67 mg, 0.18 mmol, prepared following an analogous procedure described for compound 259) in DMF (5 mL) was added 1-bromo-3-chloro-5-fluorobenzene (76 mg, 0.36 mmol), CuI (34 mg, 0.18 mmol), K₃PO₄ (38 mg, 0.18 mmol) and trans-1,2-diaminocyclohexane (21 mg, 0.18 mmol). The resulting mixture was heated to 120° C. overnight. After cooling to room temperature, solvent was removed and the residue was purified by flash column chromatography to afford tert-butyl (2R,5S)-4-(7-(3-chloro-5-fluorophenyl)-5-cyclopropyl-7H-pyrrolo[2,3-d]pyrimidin-4-yl)-2,5-dimethylpiperazine-1-carboxylate (57 mg, 63%) as a solid. LC/MS ESI (m/z): 500 (M+H)⁺. ¹H NMR (400 MHz, CDCl₃) δ 8.41 (s, 1H), 7.56-7.40 (m, 2H), 7.00 (d, J=8.1 Hz, 1H), 6.91 (s, 1H), 4.89 (s, 1H), 4.40 (s, 1H), 3.76 (m, 3H), 3.56 (d, J=11.9 Hz, 1H), 1.99 (m, 1H), 1.48 (s, 9H), 1.15 (app. t, J=6.4 Hz, 6H), 1.01 (d, J=7.9 Hz, 2H), 0.84 (m, 1H), 0.69-0.56 (m, 1H).

### Example 52. Synthesis of tert-butyl (R)-4-(3-cyclopropyl-1-(3,5-difluorophenyl)-1H-pyrazolo[3,4-d]pyrimidin-4-yl)-2-methylpiperazine-1-carboxylate (Compound 239)

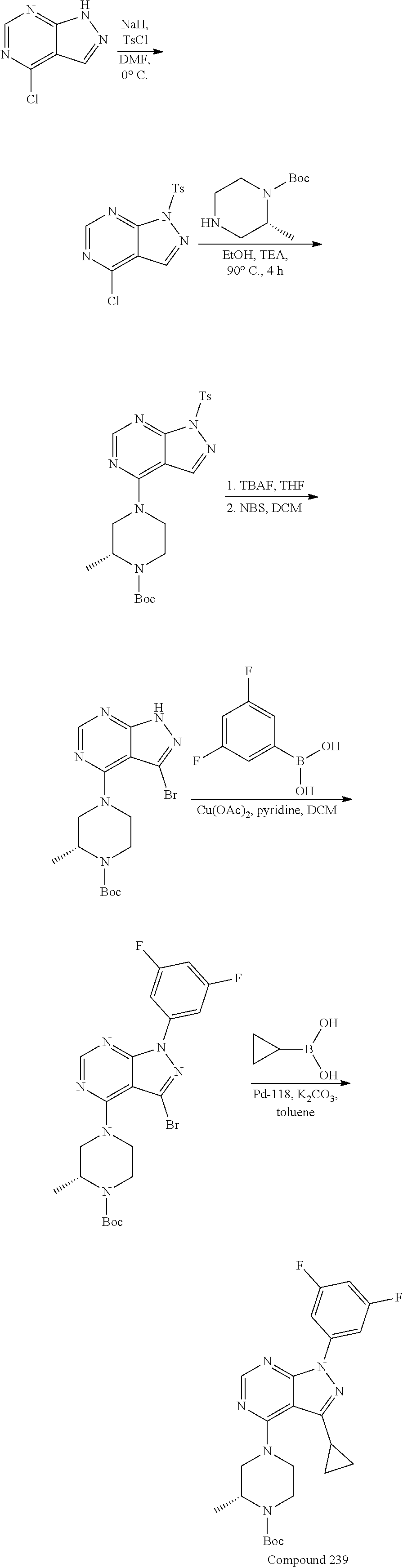

### Step 1. 4-Chloro-1-tosyl-1H-pyrazolo[3,4-d]pyrimidine

To a suspension of NaH (780 mg, 19 mmol, 60% wt.) in anhydrous DMF (10 mL) at 0° C. was added 4-chloro-1H-pyrazolo[3,4-d]pyrimidine (2.0 g, 13 mmol) in portions, followed by 4-methylbenzenesulfonyl chloride (3.7 g, 19 mmol) in portions. The resulting mixture was stirred at room temperature for 10 min. The reaction was poured into ice water and extracted twice with EtOAc. The combined organic layers were washed with brine, dried over Na₂SO₄, filtered and concentration. The residue was purified by flash column chromatography (silica gel, 0-30%, ethyl acetate in petroleum ether) to afford 4-chloro-1-tosyl-1H-pyrazolo[3,4-d]pyrimidine (1.4 g, 35%) as a light yellow solid. LC/MS ESI (m/z): 309 (M+H)⁺.

### Step 2. tert-Butyl (R)-2-methyl-4-(1-tosyl-1H-pyrazolo[3,4-d]pyrimidin-4-yl)piperazine-1-carboxylate

To a solution of 4-chloro-1-tosyl-1H-pyrazolo[3,4-d]pyrimidine (540 mg, 1.8 mmol) in EtOH (5 mL) were added tert-butyl (R)-2-methylpiperazine-1-carboxylate (420 mg, 2.1 mmol) and TEA (530 mg, 5.3 mmol). The resulting mixture was stirred at 90° C. under a N₂ atmosphere for 4 hours. After cooling to room temperature, solvent was removed and the residue was partitioned between H₂O and EtOAc. The aqueous phase was extracted twice with EtOAc. The combined organic layers were dried over Na₂SO₄, filtered and concentrated. The residue was purified by flash column chromatography (silica gel, 0-50%, ethyl acetate in petroleum ether) to afford tert-butyl (R)-2-methyl-4-(1-tosyl-1H-pyrazolo[3,4-d]pyrimidin-4-yl)piperazine-1-carboxylate (350 mg, 42%) as a yellow solid. LC/MS ESI (m/z): 473 (M+H)⁺.

### Step 3. tert-Butyl (R)-2-methyl-4-(1H-pyrazolo[3,4-d]pyrimidin-4-yl)piperazine-1-carboxylate

To a solution of tert-butyl (R)-2-methyl-4-(1-tosyl-1H-pyrazolo[3,4-d]pyrimidin-4-yl)piperazine-1-carboxylate (350 mg, 0.74 mmol) in THE (5 mL) was added TBAF (2.0 mL, 1.0M in THF). The resulting mixture was stirred at room temperature overnight. The reaction was quenched with water and extracted twice with EtOAc. The combined organic layers were washed with brine, dried over Na₂SO₄, filtered and concentrated. The residue was purified by flash column chromatography to afford tert-butyl (R)-2-methyl-4-(1H-pyrazolo[3,4-d]pyrimidin-4-yl)piperazine-1-carboxylate (220 mg, 99%) as a white solid. LC/MS ESI (m/z): 319 (M+H)⁺.

### Step 4. tert-Butyl (R)-4-(3-bromo-1H-pyrazolo[3,4-d]pyrimidin-4-yl)-2-methylpiperazine-1-carboxylate

To a solution of tert-butyl (R)-2-methyl-4-(1H-pyrazolo[3,4-d]pyrimidin-4-yl)piperazine-1-carboxylate (220 mg, 0.69 mmol) in DCM (5 mL) at 0° C. was added 1-bromopyrrolidine-2,5-dione (140 mg, 0.76 mmol) in portions. The resulting mixture was stirred at room temperature for 3 h. The reaction was quenched with water and extracted twice with DCM. The combined organic layers were washed with brine, dried over Na₂SO₄, filtered and concentrated. The residue was purified by flash column chromatography to afford tert-butyl (R)-4-(3-bromo-1H-pyrazolo[3,4-d]pyrimidin-4-yl)-2-methylpiperazine-1-carboxylate (200 mg, 73%) as a yellow solid. LC/MS ESI (m/z): 397,399 (M+H)⁺.

### Step 5. tert-Butyl (R)-4-(3-bromo-1-(3,5-difluorophenyl)-1H-pyrazolo[3,4-d]pyrimidin-4-yl)-2-methylpiperazine-1-carboxylate

To a solution of tert-butyl (R)-4-(3-bromo-1H-pyrazolo[3,4-d]pyrimidin-4-yl)-2-methylpiperazine-1-carboxylate (200 mg, 0.50 mmol) in DCM (15 mL) were added (3,5-difluorophenyl)boronic acid (160 mg, 1.0 mmol), 4 A molecular sieves (200 mg), Cu(OAc)₂ (370 mg, 2.0 mmol) and pyridine (240 mg, 3.0 mmol). The resulting mixture was stirred at room temperature under an O₂ atmosphere overnight. The reaction was quenched with NH₄₀H (10 mL) at 0° C. (ice water bath) and filtered. The filtrate was extracted twice with DCM. The combined organic layers were dried over Na₂SO₄, filtered and concentrated. The residue was purified by flash chromatography (silica gel, 0˜40% EtOAc in petroleum ether) to afford tert-butyl (R)-4-(3-bromo-1-(3,5-difluorophenyl)-1H-pyrazolo[3,4-d]pyrimidin-4-yl)-2-methylpiperazine-1-carboxylate (180 mg, 70%) as a light yellow solid. LC/MS ESI (m/z): 509, 511 (M+H)⁺.

### Step 6. tert-Butyl (R)-4-(3-cyclopropyl-1-(3,5-difluorophenyl)-1H-pyrazolo[3,4-d]pyrimidin-4-yl)-2-methylpiperazine-1-carboxylate

To a solution of tert-butyl (R)-4-(3-bromo-1-(3,5-difluorophenyl)-1H-pyrazolo[3,4-d]pyrimidin-4-yl)-2-methylpiperazine-1-carboxylate (180 mg, 0.34 mmol) in toluene (15 mL) were added cyclopropylboronic acid (91 mg, 1.1 mmol), K₂CO₃ (980 mg, 7.1 mmol) and Pd-118 (23 mg, 0.040 mmol). The resulting mixture was heated to 100° C. overnight. After cooling to room temperature, solvent was removed and the residue was purified by flash column chromatography (silica gel, 0˜50%, ethyl acetate in petroleum ether) to give crude product (80 mg), which was further purified by prep-HPLC to afford tert-butyl (R)-4-(3-cyclopropyl-1-(3,5-difluorophenyl)-1H-pyrazolo[3,4-d]pyrimidin-4-yl)-2-methylpiperazine-1-carboxylate (37 mg, 22%) as a white solid. LC/MS ESI (m/z): 471 (M+H)⁺. ¹H NMR (400 MHz, CD₃OD) δ 8.42 (s, 1H), 8.03-7.94 (m, 2H), 6.86 (tt, J=9.0, 2.3 Hz, 1H), 4.64 (d, J=9.6 Hz, 1H), 4.48-4.37 (m, 2H), 3.97 (m, 1H), 3.60 (dd, J=13.2, 3.8 Hz, 1H), 3.46-3.35 (m, 2H), 2.24-2.14 (m, 1H), 1.48 (s, 9H), 1.45-1.39 (m, 1H), 1.19 (m, 2H), 1.15 (d, J=6.7 Hz, 3H), 1.08 (ddd, J=12.2, 5.1, 2.8 Hz, 1H).

The following compound was prepared by procedures analogous to the synthesis of compound 239 using the corresponding amine and boronic acid.

| Cmpd | | |
|---|---|---|
| No. | Chemical Name | LCMS and ¹H NMR |
| 240 | tert-Butyl (S)-4-(3-cyclopropyl- | LC/MS ESI (m/z): 471 (M + H)+. ¹H NMR(400 MHz, |
| | 1-(3,5-difluorophenyl)-1H- | CD₃OD) δ 8.42 (s, 1H), 7.98 (dd, J = 9.1, 2.2 Hz, 2H), 6.86 |
| | pyrazolo[3,4-d]pyrimidin-4-yl)- | (ddd, J = 9.0, 5.7, 2.3 Hz, 1H), 4.68-4.59 (m, 1H), 4.48- |
| | 2-methylpiperazine-1- | 4.36 (m, 2H), 3.97 (m, 1H), 3.60 (dd, J = 13.2, 3.9 Hz, 1H), |
| | carboxylate | 3.38 (m, 2H), 2.24-2.13 (m, 1H), 1.48 (s, 9H), 1.45-1.40 |
| | | (m, 1H), 1.19 (m, 2H), 1.15 (d, J = 6.7 Hz, 3H), 1.08 (ddd, |
| | | J = 12.1, 5.1, 2.8 Hz, 1H). |

### Example 53. Synthesis of tert-butyl (R)-4-(7-(3,5-difluorophenyl)-5-(3-methylpyrazin-2-yl)-7H-pyrrolo[2,3-d]pyrimidin-4-yl)-2-methylpiperazine-1-carboxylate (Compound 242) and ethyl (R)-4-(7-(3,5-difluorophenyl)-5-(3-methylpyrazin-2-yl)-7H-pyrrolo[2,3-d]pyrimidin-4-yl)-2-methylpiperazine-1-carboxylate (Compound 1005)

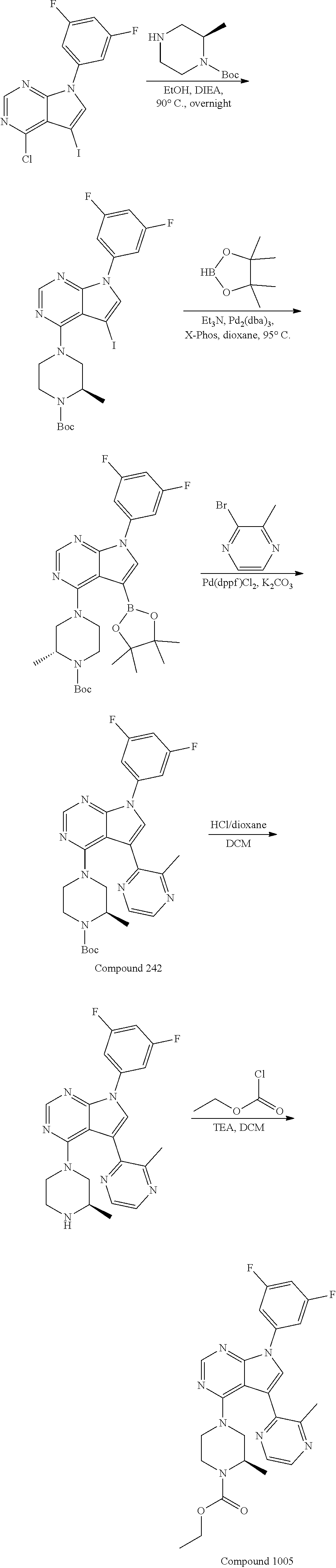

### Step 1. tert-Butyl (R)-4-(7-(3,5-difluorophenyl)-5-iodo-7H-pyrrolo[2,3-d]pyrimidin-4-yl)-2-methylpiperazine-1-carboxylate

To a solution of 4-chloro-7-(3,5-difluorophenyl)-5-iodo-7H-pyrrolo[2,3-d]pyrimidine (1.8 g, 4.6 mmol, prepared following step 1 of the procedures described for compound 193 in EtOH (5 mL) were added tert-butyl (R)-2-methylpiperazine-1-carboxylate (1.1 g, 5.5 mmol) and DIEA (1.7 g, 14 mmol). The resulting mixture was stirred at 90° C. under a N₂ atmosphere overnight. After cooling to room temperature, solvent was removed and the residue partitioned between H₂O and EtOAc. The aqueous phase was extracted twice with EtOAc. The combined organic layers were dried over Na₂SO₄, filtered and concentrated. The residue was purified by flash column chromatography (silica gel, 0-30% EtOAc in petroleum ether) to afford tert-butyl (R)-4-(7-(3,5-difluorophenyl)-5-iodo-7H-pyrrolo[2,3-d]pyrimidin-4-yl)-2-methylpiperazine-1-carboxylate (1.6 g, 62%) as a yellow solid. LC/MS ESI (m/z): 556 (M+H)⁺.

### Step 2. tert-Butyl (R)-4-(7-(3,5-difluorophenyl)-5-(4,4,5,5-tetramethyl-1,3,2-dioxaborolan-2-yl)-7H-pyrrolo[2,3-d]pyrimidin-4-yl)-2-methylpiperazine-1-carboxylate

To a solution of tert-butyl (R)-4-(7-(3,5-difluorophenyl)-5-iodo-7H-pyrrolo[2,3-d]pyrimidin-4-yl)-2-methylpiperazine-1-carboxylate (1.6 g, 2.9 mmol) in 1.4-dioxane (15 mL) were added 4,4,5,5-tetramethyl-1,3,2-dioxaborolane (1.5 g, 12 mmol), X-Phos (140 mg, 0.29 mmol), Pd₂(dba)₃ (260 mg, 0.29 mmol) and TEA (1.5 g, 14 mmol). The resulting mixture was stirred at 95° C. overnight. The reaction was quenched with H₂O, extracted twice with EtOAc. The combined organic layers were dried over Na₂SO₄, filtered and concentrated. The solvent was removed in vacuo to give tert-butyl (R)-4-(7-(3,5-difluorophenyl)-5-(4,4,5,5-tetramethyl-1,3,2-dioxaborolan-2-yl)-7H-pyrrolo[2,3-d]pyrimidin-4-yl)-2-methylpiperazine-1-carboxylate which was used in the next step without further purification. LC/MS ESI (m/z): 556 (M+H)⁺.

### Step 3. tert-Butyl (R)-4-(7-(3,5-difluorophenyl)-5-(3-methylpyrazin-2-yl)-7H-pyrrolo[2,3-d]pyrimidin-4-yl)-2-methylpiperazine-1-carboxylate

To a solution of tert-butyl (R)-4-(7-(3,5-difluorophenyl)-5-(4,4,5,5-tetramethyl-1,3,2-dioxaborolan-2-yl)-7H-pyrrolo[2,3-d]pyrimidin-4-yl)-2-methylpiperazine-1-carboxylate (1.5 g, 2.7 mmol) in dioxane (10 mL) and H₂O (2 mL) were added 2-bromo-3-methylpyrazine (1000 mg, 5.8 mmol), K₂CO₃ (2.0 g, 15 mmol) and Pd(dppf)Cl₂ (210 mg, 0.29 mmol). The resulting mixture was heated to 90° C. overnight. After cooling to room temperature, solvent was removed and the residue was purified by flash column chromatography (silica gel, 0˜50%, ethyl acetate in petroleum ether) to give crude product tert-butyl (R)-4-(7-(3,5-difluorophenyl)-5-(3-methylpyrazin-2-yl)-7H-pyrrolo[2,3-d]pyrimidin-4-yl)-2-methylpiperazine-1-carboxylate (1.1 g), 100 mg of which was further purified by prep-HPLC to afford 50 mg white solid. LC/MS ESI (m/z): 522 (M+H)⁺. ¹H NMR (400 MHz, CD₃OD) δ 8.59 (dd, J=7.3, 2.6 Hz, 2H), 8.47 (s, 1H), 7.91 (s, 1H), 7.65 (dd, J=8.4, 2.2 Hz, 2H), 7.09-6.99 (m, 1H), 4.16 (s, 1H), 3.69 (d, J=13.0 Hz, 1H), 3.57 (d, J=12.0 Hz, 1H), 3.51-3.42 (m, 1H), 2.98 (dd, J=13.3, 4.2 Hz, 1H), 2.80-2.70 (m, 1H), 2.56 (s, 3H), 2.50 (s, 1H), 1.42 (s, 9H), 1.02 (d, J=6.8 Hz, 3H).

### Step 4. (R)-7-(3,5-Difluorophenyl)-4-(3-methylpiperazin-1-yl)-5-(3-methylpyrazin-2-yl)-7H-pyrrolo[2,3-d]pyrimidine

To a solution of tert-butyl (R)-4-(7-(3,5-difluorophenyl)-5-(3-methylpyrazin-2-yl)-7H-pyrrolo[2,3-d]pyrimidin-4-yl)-2-methylpiperazine-1-carboxylate (1.0 g, 1.9 mmol) in DCM (5 m1) was added HCl (5.0 mL, 4.0M in dioxane). The resulting mixture was stirred at room temperature for 3 h. After removal of solvent, the residue was dissolved in DCM and washed with NaHCO₃ (aq.). The aqueous layer was extracted twice with DCM. The combined organic layers were washed with brine, dried over Na₂SO₄, filtered and concentrated. The residue was used in the next step directly. (R)-7-(3,5-difluorophenyl)-4-(3-methylpiperazin-1-yl)-5-(3-methylpyrazin-2-yl)-7H-pyrrolo[2,3-d]pyrimidine. LC/MS ESI (m/z): 422 (M+H)⁺.

### Step 5. Ethyl (R)-4-(7-(3,5-difluorophenyl)-5-(3-methylpyrazin-2-yl)-7H-pyrrolo[2,3-d]pyrimidin-4-yl)-2-methylpiperazine-1-carboxylate

To a solution of (R)-7-(3,5-difluorophenyl)-4-(3-methylpiperazin-1-yl)-5-(3-methylpyrazin-2-yl)-7H-pyrrolo[2,3-d]pyrimidine (100 mg, 0.24 mmol) in DCM (5 mL) at 0° C. were added TEA (72 mg, 0.71 mmol) and ethyl carbonochloridate (39 mg, 0.36 mmol) dropwise. The resulting mixture was stirred at room temperature for 2 h. The reaction was quenched with NaHCO₃ (aq.) and extracted twice with DCM. The combined organic layers were washed with brine, dried over Na₂SO₄, filtered and concentrated to give crude product which was further purified by prep-HPLC to afford ethyl (R)-4-(7-(3,5-difluorophenyl)-5-(3-methylpyrazin-2-yl)-7H-pyrrolo[2,3-d]pyrimidin-4-yl)-2-methylpiperazine-1-carboxylate (30 mg, 26%) as a white solid. LC/MS ESI (m/z): 494 (M+H)⁺. ¹H NMR (400 MHz, CD₃OD) δ 8.58 (dd, J=8.3, 2.6 Hz, 2H), 8.47 (s, 1H), 7.91 (s, 1H), 7.65 (dd, J=8.5, 2.3 Hz, 2H), 7.04 (m, 1H), 4.22 (m, 1H), 4.07 (m, 2H), 3.71 (d, J=13.3 Hz, 1H), 3.53 (m, 2H), 3.00 (dd, J=13.2, 4.1 Hz, 1H), 2.76 (td, J=12.2, 3.3 Hz, 1H), 2.56 (s, 3H), 2.51 (s, 1H), 1.22 (t, J=7.1 Hz, 3H), 1.04 (d, J=6.8 Hz, 3H).

The following compounds were prepared by procedures analogous to the synthesis of compound 1005 from the corresponding chloroformate and/or aryl halides

| Cmpd | | |
|---|---|---|
| No. | Chemical Name | LCMS and ¹H NMR |
| 251 | isopropyl (R)-4-(7-(3,5- | LC/MS ESI (m/z): 508 (M + H)⁺ ¹H NMR (400 MHz, |
| | difluorophenyl)-5-(3- | CD₃OD) δ 8.61-8.56 (m, 2H), 8.47 (s, 1H), 7.91 (s, 1H), |
| | methylpyrazin-2-yl)-7H- | 7.69-7.61 (m, 2H), 7.04 (m, J = 9.1, 2.3 Hz, 1H), 4.83- |
| | pyrrolo[2,3-d]pyrimidin-4-yl)-2- | 4.78 (m, 1H), 4.25-4.17 (m, 1H), 3.71 (dt, J = 13.1, 2.0 |
| | methylpiperazine-1-carboxylate | Hz, 1H), 3.60-3.49 (m, 2H), 2.99 (dd, J = 13.2, 4.0 Hz, |
| | | 1H), 2.76 (td, J = 12.4, 3.5 Hz, 1H), 2.56 (s, 3H), 2.51 (m, |
| | | 1H), 1.21 (dd, J = 6.2, 1.6 Hz, 6H), 1.04 (d, J = 6.8 Hz, 3H). |
| 101 | tert-butyl 4-(7-(3-chlorophenyl)- | LC/MS ESI (m/z): 491 (M + H)+. ¹H NMR (400 MHz, |
| | 5-(pyridin-2-yl)-7H-pyrrolo[2,3- | CDCl3) δ 8.71-8.67 (m, 1H), 8.53 (s, 1H), 7.84-7.76 (m, |
| | d]pyrimidin-4-yl)piperazine-1- | 2H), 7.73-7.67 (m, 2H), 7.62 (d, J = 7.9 Hz, 1H), 7.49- |
| | carboxylate | 7.44 (m, 1H), 7.39-7.34 (m, 1H), 7.28-7.24 (m, 1H), |
| | | 3.42-3.29 (m, 8H), 1.44 (s, 9H). |

### Example 54. Synthesis of 1,1,1-trifluoro-2-methylpropan-2-yl (R)-4-(7-(3,5-difluorophenyl)-5-(3-methylpyrazin-2-yl)-7H-pyrrolo[2,3-d]pyrimidin-4-yl)-2-methylpiperazine-1-carboxylate (Compound 243)

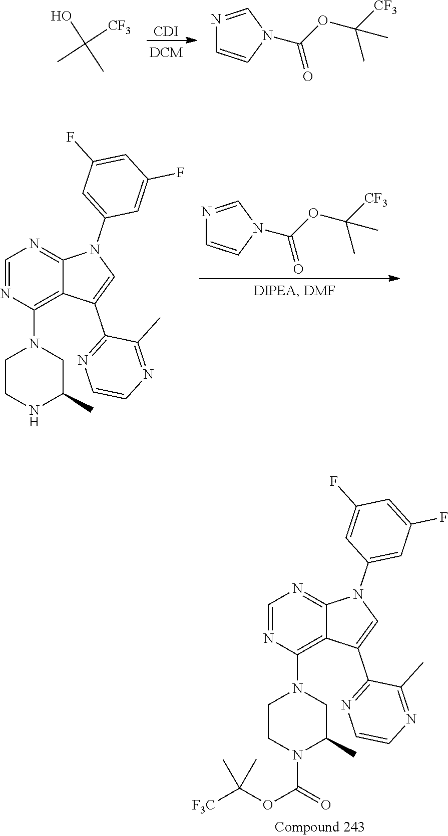

### Step 1. 1,1,1-Trifluoro-2-methylpropan-2-yl 1H-imidazole-1-carboxylate

To a solution of 1,1,1-trifluoro-2-methylpropan-2-ol (500 mg, 3.9 mmol) in DCM (10 mL) was added di(1H-imidazol-1-yl)methanone (630 mg, 3.9 mmol). The resulting mixture was stirred at room temperature overnight. The reaction was quenched with water and extracted twice with DCM. The combined organic layers were washed with brine, dried over Na₂SO₄, filtered and concentrated. The residue was used in the next step directly. LC/MS ESI (m/z): 223 (M+H)⁺.

### Step 2. 1,1,1-Trifluoro-2-methylpropan-2-yl (R)-4-(7-(3,5-difluorophenyl)-5-(3-methylpyrazin-2-yl)-7H-pyrrolo[2,3-d]pyrimidin-4-yl)-2-methylpiperazine-1-carboxylate

To a solution of (R)-7-(3,5-difluorophenyl)-4-(3-methylpiperazin-1-yl)-5-(3-methylpyrazin-2-yl)-7H-pyrrolo[2,3-d]pyrimidine (50 mg, 0.12 mmol, prepared following the procedure described for compound 1005 in DMF (3 mL) were added 1,1,1-trifluoro-2-methylpropan-2-yl 1H-imidazole-1-carboxylate (40 mg, 0.18 mmol) and DIPEA (0.04 mL, 0.22 mmol). The resulting mixture was stirred at 80° C. for 2 days under a N₂ atmosphere. The reaction was quenched with water and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-PLC to afford 1,1,1-trifluoro-2-methylpropan-2-yl (R)-4-(7-(3,5-difluorophenyl)-5-(3-methylpyrazin-2-yl)-7H-pyrrolo[2,3-d]pyrimidin-4-yl)-2-methylpiperazine-1-carboxylate (15 mg, 330%) as a white solid. LC/MS ESI (m/z): 576 (M+H)⁺. ¹H NMR (400 MHz, CD₃OD) δ 8.59 (dd, J=9.6, 2.4 Hz, 2H), 8.47 (s, 1H), 7.91 (s, 1H), 7.65 (dd, J=8.4, 2.2 Hz, 2H), 7.08-7.01 (m, 1H), 4.14 (s, 1H), 3.70 (d, J=13.3 Hz, 1H), 3.57 (d, J=11.2 Hz, 1H), 3.51-3.42 (m, 1H), 3.03 (dd, J=13.2, 4.0 Hz, 1H), 2.79 (td, J=12.3, 3.6 Hz, 1H), 2.56 (s, 3H), 2.56-2.53 (d, 1H), 1.63 (s, 6H), 1.05 (d, J=6.8 Hz, 3H).

The following compounds were prepared by procedures analogous to the synthesis of compound 243 using the corresponding alcohols.

| Cmpd | | |
|---|---|---|
| No. | Chemical Name | LCMS and ¹H NMR |
| 250 | 1-fluoro-2-methylpropan-2-yl | LC/MS ESI (m/z): 540 (M + H)⁺. ¹H NMR(400 MHz, |
| | (R)-4-(7-(3,5-difluorophenyl)-5- | CD₃OD) δ 8.61-8.56 (m, 2H), 8.47 (s, 1H), 7.91 (s, 1H), |
| | (3-methylpyrazin-2-yl)-7H- | 7.65 (dd, J = 8.4, 2.2 Hz, 2H), 7.08-7.00 (m, 1H), 4.50 (m, |
| | pyrrolo[2,3-d]pyrimidin-4-yl)-2- | 1H), 4.38 (m, 1H), 4.21-4.13 (m, 1H), 3.69 (d, J = 13.2 |
| | methylpiperazine-1-carboxylate | Hz, 1H), 3.57 (d, J = 14.0 Hz, 1H), 3.48 (dd, J = 10.2, 3.1 |
| | | Hz, 1H), 3.00 (dd, J = 13.2, 4.0 Hz, 1H), 2.77 (td, J = 12.3, |
| | | 3.5 Hz, 1H), 2.56 (s, 3H), 2.52 (m, 1H), 1.42 (d, J = 2.0 Hz, |
| | | 6H), 1.03 (d, J = 6.8 Hz, 3H). |
| 355 | 1,1,1-trifluoro-2-methylpropan- | LC/MS ESI (m/z): 552 (M + H)⁺. ¹H NMR (400 MHz, |
| | 2-yl (S)-4-(7-(4-cyanopyridin-2- | CDCl₃) δ 9.42 (s, 1H), 9.25 (s, 1H), 9.01 (s, 2H), 8.70- |
| | yl)-5-(pyrimidin-5-yl)-7H- | 8.63 (m, 2H), 8.41 (s, 1H), 7.47 (dd, J = 5.0, 1.1 Hz, 1H), |
| | pyrrolo[2,3-d]pyrimidin-4-yl)-3- | 4.07 (s, 1H), 3.93-3.67 (m, 1H), 3.61-3.49 (m, 1H), 3.47- |
| | methylpiperazine-1-carboxylate | 3.37 (m, 1H), 3.26-3.16 (m, 1H), 3.08-2.81 (m, 2H), |
| | | 1.66 (s, 6H), 1.05 (d, J = 6.6 Hz, 3H). |
| 336 | 1,1,1-trifluoro-2-methylpropan- | LC/MS ESI (m/z): 551 (M + H)⁺. ¹H NMR (400 MHz, |
| | 2-yl (S)-4-(7-(3-cyanophenyl)-5- | CD₃OD) δ 9.18 (s, 1H), 9.10 (s, 2H), 8.54 (s, 1H), 8.35- |
| | (pyrimidin-5-yl)-7H-pyrrolo[2,3- | 8.31 (m, 1H), 8.22-8.18 (m, 1H), 8.08 (s, 1H), 7.82-7.74 |
| | d]pyrimidin-4-yl)-3- | (m, 2H), 4.15-4.04 (m, 1H), 3.89-3.68 (m, 1H), 3.58- |
| | methylpiperazine-1-carboxylate | 3.44 (m, 2H), 3.27-3.15 (m, 1H), 3.12-2.87 (m, 2H), |
| | | 1.66 (s, 6H), 1.02 (d, J = 6.6 Hz, 3H). |
| 372 | 2,2,2-trifluoroethyl (S)-4-(7-(4- | LC/MS ESI (m/z): 523 (M + H)⁺. ¹H NMR (400 MHz, |
| | cyanopyridin-2-yl)-5-(pyridin-2- | CDCl₃) δ 9.39 (s, 1H), 8.74 (d, J = 4.6 Hz, 1H), 8.67-8.54 |
| | yl)-7H-pyrrolo[2,3-d]pyrimidin- | (m, 3H), 7.90-7.80 (m, 1H), 7.68-7.61 (m, 1H), 7.42 (dd, |
| | 4-yl)-3-methylpiperazine-1- | J = 5.0, 1.2 Hz, 1H), 7.37-7.30 (m, 1H), 4.58-4.32 (m, |
| | carboxylate | 3H), 3.95-3.85 (m, 1H), 3.67 (d, J = 13.3 Hz, 1H), 3.59 (d, |
| | | J = 15.3 Hz, 1H), 3.22-2.79 (m, 3H), 1.14-0.94 (m, 3H). |
| 371 | 2,2,2-trifluoroethyl (S)-4-(7-(4- | ¹H NMR (400 MHz, CD₃OD) δ 9.30-9.27 (m, 1H), 8.70 |
| | cyanopyridin-2-yl)-5-phenyl- | (dd, J = 5.0, 0.8 Hz, 1H), 8.55 (s, 1H), 8.25 (s, 1H), 7.61- |
| | 7H-pyrrolo[2,3-d]pyrimidin-4- | 7.55 (m, 3H), 7.53-7.49 (m, 2H), 7.44-7.39 (m, 1H), |
| | yl)-3-methylpiperazine-1- | 4.66-4.47 (m, 2H), 4.22-4.16 (m, 1H), 3.86-3.78 (m, |
| | carboxylate | 1H), 3.60-3.50 (m, 2H), 3.19-3.11 (m, 1H), 2.92 (m, |
| | | 2H), 0.96 (d, J = 6.7 Hz, 3H). |

### Example 55. Synthesis of tert-butyl (S)-4-(7-(3,5-difluorophenyl)-5-(3-methylpyrazin-2-yl)-7H-pyrrolo[2,3-d]pyrimidin-4-yl)-3-methylpiperazine-1-carboxylate (Compound 246)

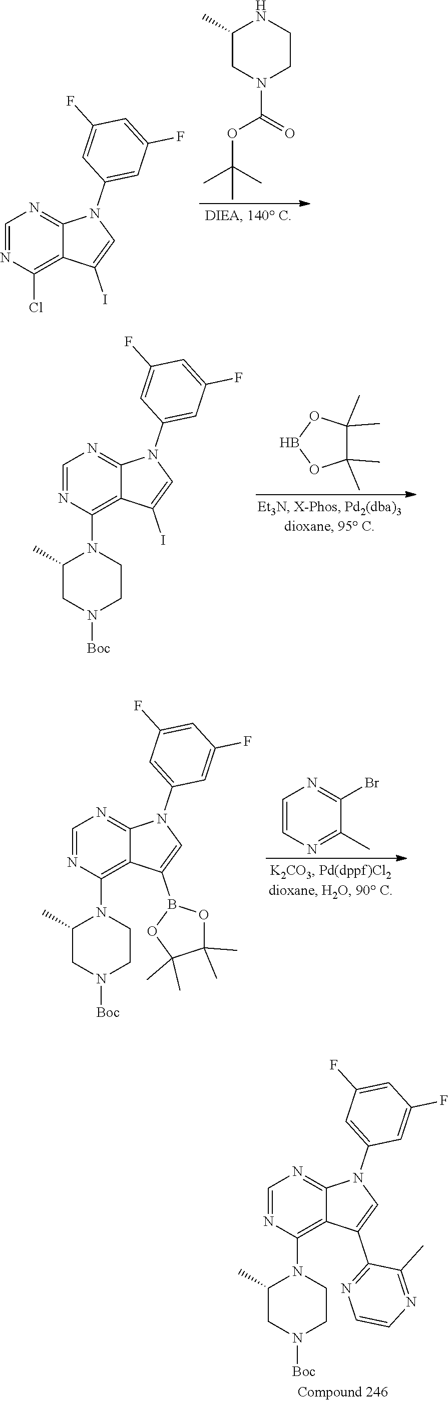

### Step 1. tert-Butyl (S)-4-(7-(3,5-difluorophenyl)-5-iodo-7H-pyrrolo[2,3-d]pyrimidin-4-yl)-3-methylpiperazine-1-carboxylate

To a solution of 4-chloro-7-(3,5-difluorophenyl)-5-iodo-7H-pyrrolo[2,3-d]pyrimidine (2.0 g, 5.0 mmol) in DIEA (50 mL) was added tert-butyl (S)-3-methylpiperazine-1-carboxylate (1.5 g, 7.5 mmol). The resulting reaction mixture was stirred at 140° C. under a N₂ atmosphere for 13 h. After cooling to room temperature, the reaction mixture was concentrated under reduced pressure. The residue was extracted twice with EtOAc. The combined organic layers were dried over Na₂SO₄, filtered, and concentrated under reduced pressure. The crude product was purified by flash column chromatography (silica gel, 0˜40%, ethyl acetate in petroleum ether) to afford tert-butyl (S)-4-(7-(3,5-difluorophenyl)-5-iodo-7H-pyrrolo[2,3-d]pyrimidin-4-yl)-3-methylpiperazine-1-carboxylate (1.9 g, 66% yield) as white solid. LC/MS ESI (m/z): 556 (M+H)⁺.

### Step 2. tert-Butyl (S)-4-(7-(3,5-difluorophenyl)-5-(4,4,5,5-tetramethyl-1,3,2-dioxaborolan-2-yl)-7H-pyrrolo[2,3-d]pyrimidin-4-yl)-3-methylpiperazine-1-carboxylate

To a solution of tert-butyl (S)-4-(7-(3,5-difluorophenyl)-5-iodo-7H-pyrrolo[2,3-d]pyrimidin-4-yl)-3-methylpiperazine-1-carboxylate (1.9 g, 3.4 mmol) in dioxane (25 mL) were added XPhos (160 mg, 0.33 mmol), Pd₂(dba)₃ (300 mg, 0.33 mmol), TEA (1.7 g, 2.4 mL, 17 mmol) and 4,4,5,5-tetramethyl-1,3,2-dioxaborolane (1.7 g, 2.0 mL, 14 mmol). The resulting reaction mixture was stirred at 95° C. under a N₂ atmosphere overnight. After cooling to room temperature, the reaction mixture was quenched with ice water and extracted twice with DCM. The combined organic layers were dried over Na₂SO₄, filtered, and concentrated under reduced pressure to afford the crude product tert-butyl (S)-4-(7-(3,5-difluorophenyl)-5-(4,4,5,5-tetramethyl-1,3,2-dioxaborolan-2-yl)-7H-pyrrolo[2,3-d]pyrimidin-4-yl)-3-methylpiperazine-1-carboxylate (2.0 g) as yellow oil. It was used directly in the next step. LC/MS ESI (m/z): 556 (M+H)⁺.

### Step 3. tert-Butyl (S)-4-(7-(3,5-difluorophenyl)-5-(3-methylpyrazin-2-yl)-7H-pyrrolo[2,3-d]pyrimidin-4-yl)-3-methylpiperazine-1-carboxylate

To a solution of tert-butyl (S)-4-(7-(3,5-difluorophenyl)-5-(4,4,5,5-tetramethyl-1,3,2-dioxaborolan-2-yl)-7H-pyrrolo[2,3-d]pyrimidin-4-yl)-3-methylpiperazine-1-carboxylate (280 mg, 0.50 mmol) in dioxane (1 mL) and H₂O (90 mL) were added K₂CO₃ (690 mg, 5.0 mmol), Pd(dppf)Cl₂ (36 mg, 0.050 mmol) and 2-bromo-3-methylpyrazine (170 mg, 1.0 mmol). The resulting reaction mixture was stirred at 90° C. under a N₂ atmosphere overnight. After cooling to room temperature, solvent was removed under reduced pressure. The residue was extracted twice with EtOAc. The combined organic layers were dried over Na₂SO₄, filtered, and concentrated under reduced pressure. The residue was purified by flash column chromatography (silica gel, 0˜10%, MeOH in DCM) to afford crude product (220 mg, 84%). 60 mg of crude product was purified by prep-HPLC (Gilson, C18, MeCN in H₂O) to afford tert-butyl (S)-4-(7-(3,5-difluorophenyl)-5-(3-methylpyrazin-2-yl)-7H-pyrrolo[2,3-d]pyrimidin-4-yl)-3-methylpiperazine-1-carboxylate (32 mg) as a white solid. LC/MS ESI (m/z): 522 (M+H)⁺. ¹H NMR (400 MHz, CD₃OD) δ 8.60 (d, J=2.4 Hz, 1H), 8.58 (d, J=2.6 Hz, 1H), 8.48 (s, 1H), 7.90 (s, 1H), 7.71-7.59 (m, 2H), 7.04 (tt, J=9.0, 2.3 Hz, 1H), 4.03 (s, 1H), 3.73 (d, J=12.9 Hz, 1H), 3.50 (t, J=14.6 Hz, 2H), 3.05 (t, J=11.2 Hz, 1H), 2.69 (m, 2H), 2.55 (s, 3H), 1.42 (s, 9H), 0.92 (d, J=7.7 Hz, 3H).

### Example 56. Synthesis of 1,1,1-trifluoro-2-methylpropan-2-yl (S)-4-(7-(3,5-difluorophenyl)-5-(3-methylpyrazin-2-yl)-7H-pyrrolo[2,3-d]pyrimidin-4-yl)-3-methylpiperazine-1-carboxylate (Compound 247)

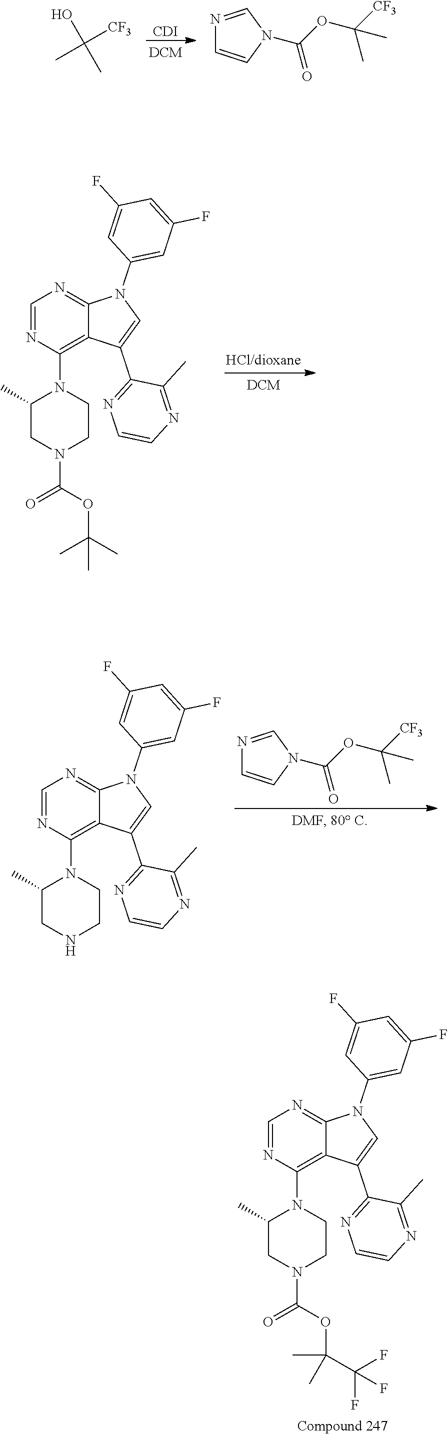

### Step 1. 1,1,1-Trifluoro-2-methylpropan-2-yl 1H-imidazole-1-carboxylate

To a solution of 1,1,1-trifluoro-2-methylpropan-2-ol (500 mg, 3.9 mmol) in DCM (10 mL) was added di(1H-imidazol-1-yl)methanone (630 mg, 3.9 mmol). The resulting mixture was stirred at room temperature overnight. The reaction was quenched with water and extracted twice with DCM. The combined organic layers were washed with brine, dried over Na₂SO₄, filtered and concentrated to afford 1,1,1-trifluoro-2-methylpropan-2-yl 1H-imidazole-1-carboxylate (640 mg, 73% yield) as a white solid. LC/MS ESI (m/z): 223 (M+H)⁺.

### Step 2. (S)-7-(3,5-Difluorophenyl)-4-(2-methylpiperazin-1-yl)-5-(3-methylpyrazin-2-yl)-7H-pyrrolo[2,3-d]pyrimidine

To a solution of tert-butyl (S)-4-(7-(3,5-difluorophenyl)-5-(3-methylpyrazin-2-yl)-7H-pyrrolo[2,3-d]pyrimidin-4-yl)-3-methylpiperazine-1-carboxylate (250 mg, 0.48 mmol, prepared following the procedure of compound 246) in DCM (5 mL) was added HCl/dioxane (5.0 mL, 4.0 M). The resulting mixture was stirred at room temperature for 3 h. After removal of solvent, the residue was dissolved in DCM and washed with aqueous NaHCO₃. The aqueous layer was extracted twice with DCM. The combined organic layers were washed with brine, dried over Na₂SO₄, filtered and concentrated. The residue was used in the next step directly. (S)-7-(3,5-difluorophenyl)-4-(2-methylpiperazin-1-yl)-5-(3-methylpyrazin-2-yl)-7H-pyrrolo[2,3-d]pyrimidine (180 mg, 89%). LC/MS ESI (m/z): 422 (M+H)⁺.

### Step 3. 1,1,1-Trifluoro-2-methylpropan-2-yl (S)-4-(7-(3,5-difluorophenyl)-5-(3-methylpyrazin-2-yl)-7H-pyrrolo[2,3-d]pyrimidin-4-yl)-3-methylpiperazine-1-carboxylate

To a solution of (S)-7-(3,5-difluorophenyl)-4-(2-methylpiperazin-1-yl)-5-(3-methylpyrazin-2-yl)-7H-pyrrolo[2,3-d]pyrimidine (60 mg, 0.14 mmol) in DMF (5 mL) was added 1,1,1-trifluoro-2-methylpropan-2-yl 1H-imidazole-1-carboxylate (64 mg, 0.28 mmol). The resulting mixture was stirred at 80° C. overnight under a N₂ atmosphere. The reaction was quenched with water and extracted twice with EtOAc. The combined organic layers were washed with brine, dried over Na₂SO₄, filtered and concentrated. The residue was purified by prep-HPLC to afford 1,1,1-trifluoro-2-methylpropan-2-yl (S)-4-(7-(3,5-difluorophenyl)-5-(3-methylpyrazin-2-yl)-7H-pyrrolo[2,3-d]pyrimidin-4-yl)-3-methylpiperazine-1-carboxylate (23 mg, 29%) as a white solid. LC/MS ESI (m/z): 576 (M+H)⁺. ¹H NMR (400 MHz, MeOD) δ 8.60 (d, J=2.4 Hz, 1H), 8.58 (d, J=2.6 Hz, 1H), 8.48 (s, 1H), 7.91 (s, 1H), 7.71-7.60 (m, 2H), 7.04 (tt, J=9.0, 2.3 Hz, 1H), 4.06 (s, 1H), 3.71 (dd, J=44.1, 12.8 Hz, 1H), 3.49 (m, 2H), 3.07 (t, J=12.6 Hz, 1H), 2.91-2.59 (m, 2H), 2.55 (s, 3H), 1.64 (s, 6H), 0.92 (d, J=8.2 Hz, 3H).

The following compound was prepared by the procedures analogous to the synthesis of compound 247 from the corresponding alcohol.

| Cmpd | | |
|---|---|---|
| No. | Chemical Name | LCMS and ¹H NMR |
| 248 | 1-fluoro-2-methylpropan-2-yl | LC/MS ESI (m/z): 540 (M + H)⁺. ¹H NMR (400 MHz, |
| | (S)-4-(7-(3,5-difluorophenyl)-5- | MeOD) δ 8.59 (dd, J = 7.4, 2.4 Hz, 2H), 8.48 (s, 1H), 7.90 |
| | (3-methylpyrazin-2-yl)-7H- | (s, 1H), 7.72-7.59 (m, 2H), 7.04 (tt, J = 9.0, 2.3 Hz, 1H), |
| | pyrrolo[2,3-d]pyrimidin-4-yl)-3- | 4.44 (d, J = 47.6 Hz, 2H), 4.08-3.99 (m, 1H), 3.75 (d, J = |
| | methylpiperazine-1-carboxylate | 13.1 Hz, 1H), 3.51 (t, J = 15.0 Hz, 2H), 3.05 (dd, J = 17.8, |
| | | 7.7 Hz, 1H), 2.70 (d, J = 9.0 Hz, 2H), 2.55 (s, 3H), 1.42 (d, |
| | | J = 1.9 Hz, 6H), 0.92 (t, J = 7.8 Hz, 3H). |

### Example 57. Synthesis of ethyl (S)-4-(7-(3,5-difluorophenyl)-5-(3-methylpyrazin-2-yl)-7H-pyrrolo[2,3-d]pyrimidin-4-yl)-3-methylpiperazine-1-carboxylate (Compound 1001)

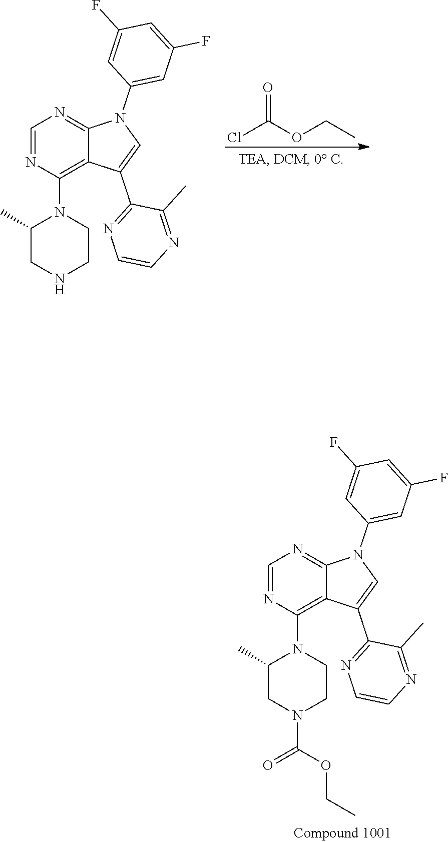

### Step 1. Ethyl (S)-4-(7-(3,5-difluorophenyl)-5-(3-methylpyrazin-2-yl)-7H-pyrrolo[2,3-d]pyrimidin-4-yl)-3-methylpiperazine-1-carboxylate

To a solution of (S)-7-(3,5-difluorophenyl)-4-(2-methylpiperazin-1-yl)-5-(3-methylpyrazin-2-yl)-7H-pyrrolo[2,3-d]pyrimidine (27 mg, 0.064 mmol, prepared following step 2 of the procedures described for compound 247) in DCM (5 mL) at 0° C. was added TEA (0.50 mL), followed by ethyl carbonochloridate (0.50 mL) dropwise. The resulting mixture was stirred at 0° C. for 30 min. The solvent was removed under reduced pressure. The residue was purified by prep-HPLC to afford ethyl (S)-4-(7-(3,5-difluorophenyl)-5-(3-methylpyrazin-2-yl)-7H-pyrrolo[2,3-d]pyrimidin-4-yl)-3-methylpiperazine-1-carboxylate (18 mg, 55% yield) as a white solid. LC/MS ESI (m/z): 494 (M+H)⁺. ¹H NMR (400 MHz, MeOD) δ 8.6 (d, J=2.6 Hz, 1H), 8.6 (d, J=2.6 Hz, 1H), 8.5 (s, 1H), 7.9 (s, 1H), 7.7-7.6 (m, 2H), 7.0 (tt, J=9.0, 2.3 Hz, 1H), 4.2-4.0 (m, 3H), 3.8 (d, J=13.3 Hz, 1H), 3.5 (dd, J=23.1, 13.3 Hz, 2H), 3.1 (m, 1H), 2.9-2.6 (m, 2H), 2.5 (s, 3H), 1.2 (t, J=7.0 Hz, 3H), 0.9 (d, J=6.6 Hz, 3H).

The following compound was prepared by procedures analogous to the synthesis of compound 1001 using the corresponding chloroformate.

| Cmpd | | |
|---|---|---|
| No. | Chemical Name | LCMS and ¹H NMR |
| 249 | Isopropyl (S)-4-(7-(3,5- | LC/MS ESI (m/z): 508 (M + H)⁺. ¹H NMR (400 MHz, |
| | difluorophenyl)-5-(3- | MeOD) δ 8.6 (d, J = 2.6 Hz, 1H), 8.6 (d, J = 2.6 Hz, 1H), |
| | methylpyrazin-2-yl)-7H- | 8.5 (s, 1H), 7.9 (s, 1H), 7.7-7.6 (m, 2H), 7.0 (tt, J = 9.0, |
| | pyrrolo[2,3-d]pyrimidin-4-yl)-3- | 2.3 Hz, 1H), 4.8 (m, 1H), 4.1-4.0 (m, 1H), 3.8 (d, J = 12.7 |
| | methylpiperazine-1-carboxylate | Hz, 1H), 3.5 (dd, J = 24.2, 13.3 Hz, 2H), 3.1 (m, 1H), 2.8- |
| | | 2.6 (m, 2H), 2.6 (s, 3H), 1.2-1.2 (m, 6H), 0.9 (d, J = 6.7 |
| | | Hz, 3H). |

### Example 58. Synthesis of tert-butyl (R)-4-(7-(3-chlorophenyl)-5-(2-oxopyrrolidin-1-yl)-7H-pyrrolo[2,3-d]pyrimidin-4-yl)-2-methylpiperazine-1-carboxylate (Compound 252)

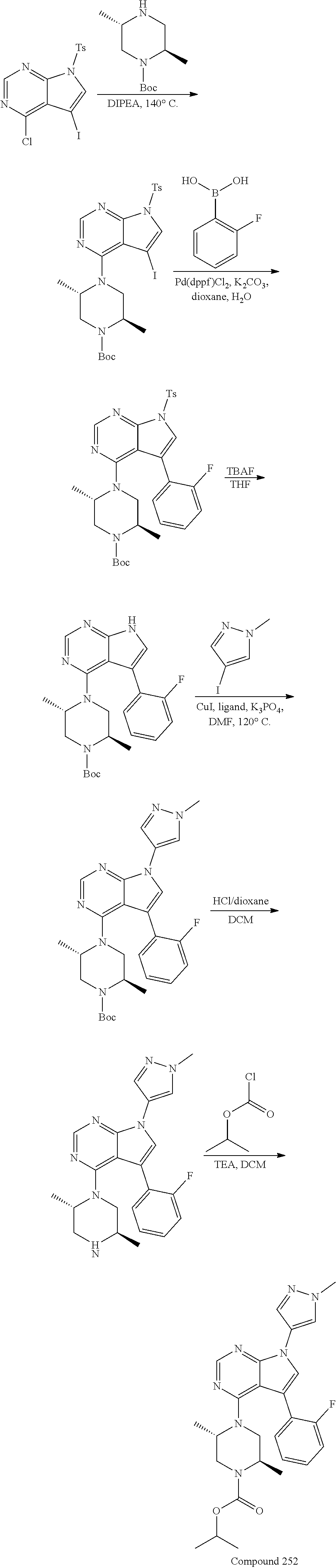

### Step 1. tert-Butyl (2R,5S)-4-(5-iodo-7-tosyl-7H-pyrrolo[2,3-d]pyrimidin-4-yl)-2,5-dimethylpiperazine-1-carboxylate

To a solution of 4-chloro-5-iodo-7-tosyl-7H-pyrrolo[2,3-d]pyrimidine (2.5 g, 5.8 mmol, prepared following step 1 of the procedures for compound 192 in DIPEA (20 mL) was added tert-butyl (2R,5S)-2,5-dimethylpiperazine-1-carboxylate (2.5 g, 12 mmol). The resulting mixture was heated to 140° C. for 3 h. After cooling to room temperature, solvent was removed and the residue was purified by flash column chromatography (silica gel, 0˜30%, ethyl acetate in petroleum ether) to afford tert-butyl (2R,5S)-4-(5-iodo-7-tosyl-7H-pyrrolo[2,3-d]pyrimidin-4-yl)-2,5-dimethylpiperazine-1-carboxylate (3.1 g, 87%) as a white solid. LC/MS ESI (m/z): 612 (M+H)⁺.

### Step 2. tert-Butyl (2R,5S)-4-(5-(2-fluorophenyl)-7-tosyl-7H-pyrrolo[2,3-d]pyrimidin-4-yl)-2,5-dimethylpiperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (3.1 g, 5.1 mmol) in dioxane-water (30 mL, 5:1 v/v) were added (2-fluorophenyl)boronic acid (1.4 g, 10 mmol), K₂CO₃ (2.1 g, 15 mmol) and Pd(dppf)Cl₂ (370 mg, 0.51 mmol). The resulting mixture was heated to 90° C. overnight. After cooling to room temperature, the reaction was filtered. The filtrate was partitioned between EtOAc and water. The aqueous phase was extracted twice with EtOAc. The combined organic layers were washed with brine, dried over Na₂SO₄, filtered and concentrated, the residue was purified by flash chromatography (silica gel, 0˜50% EtOAc in petroleum ether) to afford tert-butyl (2R,5S)-4-(5-(2-fluorophenyl)-7-tosyl-7H-pyrrolo[2,3-d]pyrimidin-4-yl)-2,5-dimethylpiperazine-1-carboxylate (2.5 g, 85%) as a white solid. LC/MS ESI (m/z): 580 (M+H)⁺.

### Step 3. tert-Butyl (2R,5S)-4-(5-(2-fluorophenyl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7-tosyl-7H-pyrrolo[2,3-d]pyrimidin-4-yl)-2,5-dimethylpiperazine-1-carboxylate (2.5 g, 4.3 mmol) in THE (10 mL) were added TBAF (20 mL, 1.0M in THF). The resulting mixture was stirred at room temperature overnight. Solvent was removed and the residue was dissolved in DCM, washed with NH₄Cl (aq.) and brine and dried over Na₂SO₄. After filtration, the filtrate was concentrated, the residue was purified by flash column chromatography (silica gel, 0˜80% ethyl acetate in petroleum ether) to afford tert-butyl (2R,5S)-4-(5-(2-fluorophenyl)-7H-pyrrolo[2,3-d]pyrimidin-4-yl)-2,5-dimethylpiperazine-1-carboxylate (1.2 g, 65%). LC/MS ESI (m/z): 426 (M+H)⁺.

### Step 3. tert-Butyl (2R,5S)-4-(5-(2-fluorophenyl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7H-pyrrolo[2,3-d]pyrimidin-4-yl)-2,5-dimethylpiperazine-1-carboxylate (1.2 g, 2.8 mmol) in DMF (30 mL) were added 4-iodo-1-methyl-1H-pyrazole (1.8 g, 8.5 mmol), trans-cyclohexane-1,2-diamine (97 mg, 0.84 mmol), CuI (270 mg, 1.4 mmol) and K₃PO₄ (1.8 g, 8.4 mmol). The resulting mixture was heated to 120° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to give tert-butyl (2R,5S)-4-(5-(2-fluorophenyl)-7-(1-methyl-1H-pyrazol-4-yl)-7H-pyrrolo[2,3-d]pyrimidin-4-yl)-2,5-dimethylpiperazine-1-carboxylate (400 mg, 28%) as a white solid. LC/MS ESI (m/z): 506 (M+H)⁺.

### Step 5. 4-((2S,5R)-2,5-Dimethylpiperazin-1-yl)-5-(2-fluorophenyl)-7-(I-methyl-1H-pyrazol-4-yl)-7H-pyrrolo[2,3-d]pyrimidine

To a solution of tert-butyl (2R,5S)-4-(5-(2-fluorophenyl)-7-(1-methyl-1H-pyrazol-4-yl)-7H-pyrrolo[2,3-d]pyrimidin-4-yl)-2,5-dimethylpiperazine-1-carboxylate (400 mg, 0.79 mmol) in DCM (5 mL) was added HCl (3.0 mL, 4.0 M in dioxane). The resulting mixture was stirred at room temperature for 2 h. After removal of solvent, the residue was diluted with DCM, washed with NaHCO₃ (aq.). The organic layer was extracted twice with DCM. The combined organic layers were washed with brine, dried over Na₂SO₄, filtered and concentrated. The residue was used in the next step directly. 4-((2S,5R)-2,5-dimethylpiperazin-1-yl)-5-(2-fluorophenyl)-7-(1-methyl-1H-pyrazol-4-yl)-7H-pyrrolo[2,3-d]pyrimidine (400 mg). LC/MS ESI (m/z): 406 (M+H)⁺.

### Step 6. Isopropyl (2R,5S)-4-(5-(2-fluorophenyl)-7-(I-methyl-1H-pyrazol-4-yl)-7H-pyrrolo[2,3-d]pyrimidin-4-yl)-2,5-dimethylpiperazine-1-carboxylate

To a solution of 4-((2S,5R)-2,5-dimethylpiperazin-1-yl)-5-(2-fluorophenyl)-7-(1-methyl-1H-pyrazol-4-yl)-7H-pyrrolo[2,3-d]pyrimidine (90 mg, 0.22 mmol) in DCM (3 mL) at 0° C. were added TEA (66 mg, 0.66 mmol), followed by isopropyl carbonochloridate (81 mg, 0.66 mmol) dropwise. The resulting mixture was stirred at room temperature overnight. The reaction was quenched with water and extracted twice with DCM. The combined organic layers were washed with NaHCO₃ (aq.), dried over Na₂SO₄, filtered and concentrated. The residue was purified by flash column chromatography (silica gel, 0˜30%, ethyl acetate in petroleum ether) to give crude product which was further purified by prep-HPLC to afford isopropyl (2R,5S)-4-(5-(2-fluorophenyl)-7-(1-methyl-1H-pyrazol-4-yl)-7H-pyrrolo[2,3-d]pyrimidin-4-yl)-2,5-dimethylpiperazine-1-carboxylate (25 mg, 22%) as a white solid. LC/MS ESI (m/z): 492 (M+H)⁺. ¹H NMR (400 MHz, CD₃OD) δ 8.39 (s, 1H), 8.25 (s, 1H), 7.97 (s, 1H), 7.60 (s, 1H), 7.53 (t, J=7.6 Hz, 1H), 7.47-7.42 (m, 1H), 7.32 (t, J=7.5 Hz, 1H), 7.26 (t, J=9.1 Hz, 1H), 4.82 (d, J=5.7 Hz, 1H), 4.17 (s, 2H), 3.98 (s, 3H), 3.42 (d, J=13.5 Hz, 1H), 3.32 (d, J=4.2 Hz, 1H), 3.25 (d, J=4.0 Hz, 1H), 2.89-2.77 (m, 1H), 1.21 (s, 6H), 1.10 (d, J=6.8 Hz, 3H), 0.90 (d, J=6.7 Hz, 3H).

The following compounds were prepared by procedures analogous to the synthesis of compound 252 using the corresponding chloroformates and coupling partners (aryl halides or boronic acids).

| Cmpd | | |
|---|---|---|
| No. | Chemical Name | LCMS and ¹H NMR |
| 132 | Ethyl (2R,5S)-4-(7-(3- | LC/MS ESI (m/z): 454 (M + H)⁺. ¹H NMR (400 MHz, |
| | chlorophenyl)-5-cyclopropyl- | CDCl3) δ 8.43 (s, 1H), 7.70-7.64 (m, 1H), 7.60 (d, J = 8.1 |
| | 7H-pyrrolo[2,3-d]pyrimidin-4- | Hz, 1H), 7.45-7.39 (m, 1H), 7.30 (d, J = 8.3 Hz, 1H), 6.93 |
| | yl)-2,5-dimethylpiperazine-1- | (s, 1H), 4.97-4.87 (m, 1H), 4.56-4.38 (m, 1H), 4.24- |
| | carboxylate | 4.14 (m, 2H), 3.87-3.72 (m, 3H), 3.66-3.58 (m, 1H), |
| | | 2.04-1.99 (m, 1H), 1.29 (t, J = 7.1 Hz, 3H), 1.21 (d, J = |
| | | 6.8 Hz, 3H), 1.16 (d, J = 6.7 Hz, 3H), 1.05-1.00 (m, 2H), |
| | | 0.89-0.85 (m, 1H), 0.68-0.62 (m, 1H). |
| 133 | Ethyl (2R,5S)-4-(7-(3- | LC/MS ESI (m/z): 491 (M + H)+. ¹H NMR (400 MHz, |
| | chlorophenyl)-5-(pyridin-3-yl)- | CDCl3) δ 8.85 (d, J = 1.6 Hz, 1H), 8.63 (d, J = 3.4 Hz, 1H), |
| | 7H-pyrrolo[2,3-d]pyrimidin-4- | 8.56 (s, 1H), 7.91-7.85 (m, 1H), 7.82-7.74 (m, 1H), 7.71- |
| | yl)-2,5-dimethylpiperazine-1- | 7.64 (m, 1H), 7.53-7.44 (m, 1H), 7.43-7.35 (m, 3H), |
| | carboxylate | 4.43-4.19 (m, 1H), 4.16-4.04 (m, 3H), 3.42-3.28 (m, |
| | | 3H), 3.08-2.87 (m, 1H), 1.24 (t, J = 7.9 Hz, 3H), 1.15 (d, |
| | | J = 6.8 Hz, 3H), 0.97 (d, J = 6.6 Hz, 3H). |

### Example 59. Synthesis of 2,2,2-trifluoroethyl (2R,5S)-4-(5-(2-fluorophenyl)-7-(1-methyl-1H-pyrazol-4-yl)-7H-pyrrolo[2,3-d]pyrimidin-4-yl)-2,5-dimethylpiperazine-1-carboxylate (Compound 254)

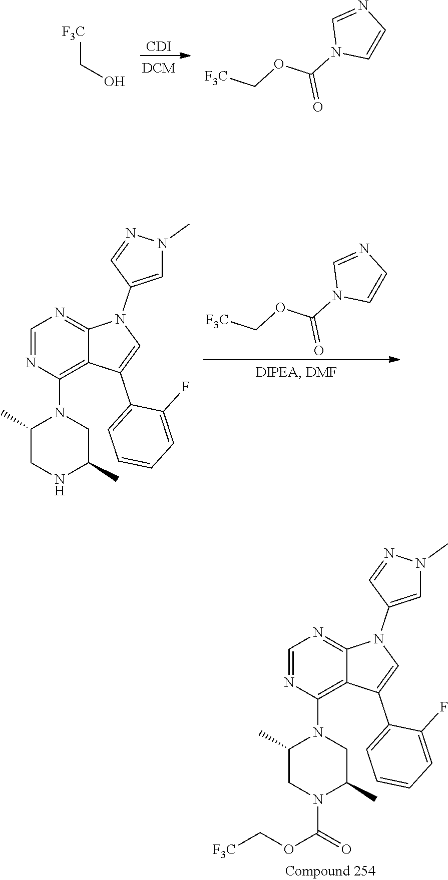

### Step 1. 2,2,2-Trifluoroethyl 1H-imidazole-1-carboxylate

To a solution of 2,2,2-trifluoroethan-1-ol (1.0 g, 10 mmol) in DCM (10 mL) was added CDI (1. 8 g, 11 mmol). The resulting mixture was stirred at room temperature overnight. The reaction was quenched with water and extracted twice with DCM. The combined organic layers were washed with brine, dried over Na₂SO₄, filtered and concentrated to afford 1,1,1-trifluoro-2-methylpropan-2-yl 1H-imidazole-1-carboxylate which was used in the next step directly. LC/MS ESI (m/z): 195 (M+H)⁺.

### Step 2. 2,2,2-Trifluoroethyl (2R,5S)-4-(5-(2-fluorophenyl)-7-(I-methyl-1H-pyrazol-4-yl)-7H-pyrrolo[2,3-d]pyrimidin-4-yl)-2,5-dimethylpiperazine-1-carboxylate

To a solution of 4-((2S,5R)-2,5-dimethylpiperazin-1-yl)-5-(2-fluorophenyl)-7-(1-methyl-1H-pyrazol-4-yl)-7H-pyrrolo[2,3-d]pyrimidine (70 mg, 0.18 mmol, prepared following the first 5 steps of the procedures described for compound 252) in DMF (5 mL) were added 2,2,2-trifluoroethyl 1H-imidazole-1-carboxylate (70 mg, 0.36 mmol) and DIPEA (70 mg, 0.54 mmol). The resulting mixture was stirred at 80° C. for overnight under a N₂ atmosphere. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford 2,2,2-trifluoroethyl (2R,5S)-4-(5-(2-fluorophenyl)-7-(1-methyl-1H-pyrazol-4-yl)-7H-pyrrolo[2,3-d]pyrimidin-4-yl)-2,5-dimethylpiperazine-1-carboxylate (30 mg, 33%) as a white solid. LC/MS ESI (m/z): 532 (M+H)⁺. ¹H NMR (400 MHz, CD₃OD) δ 8.41 (s, 1H), 8.26 (s, 1H), 7.97 (d, J=0.6 Hz, 1H), 7.61 (s, 1H), 7.56-7.51 (m, 1H), 7.48-7.42 (m, 1H), 7.34-7.30 (m, 1H), 7.29-7.24 (m, 1H), 4.66-4.46 (m, 2H), 4.19 (s, 2H), 3.98 (s, 3H), 3.47-3.42 (m, 1H), 3.35 (d, J=2.0 Hz, 1H), 3.30-3.26 (m, 1H), 2.97-2.83 (m, 1H), 1.13 (d, J=6.8 Hz, 3H), 0.92 (d, J=6.7 Hz, 3H).

### Example 60. Synthesis of tert-butyl (R)-4-(5-cyclopropyl-7-(3,5-difluorophenyl)-7H-pyrrolo[2,3-d]pyrimidin-4-yl)-2-ethylpiperazine-1-carboxylate (Compound 256)

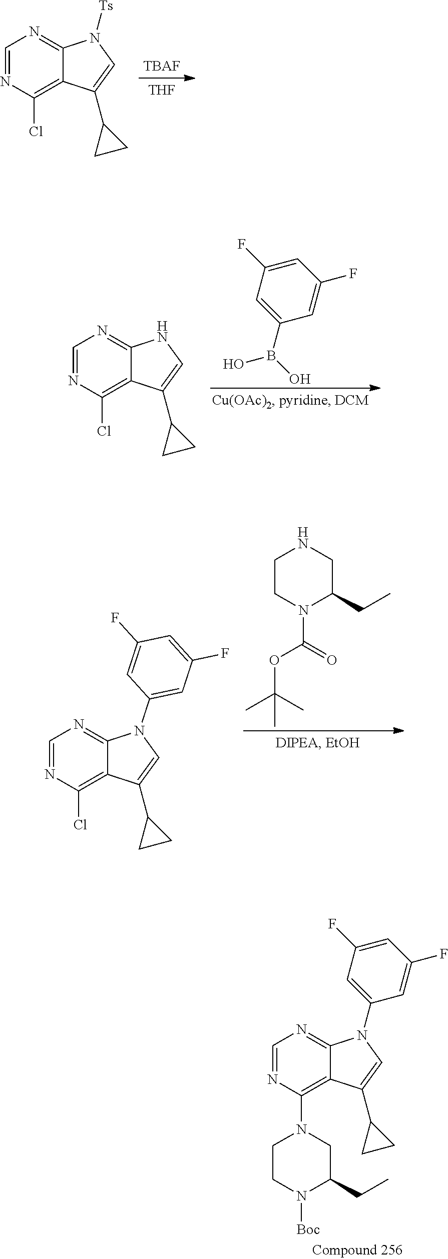

### Step 1. 4-Chloro-5-cyclopropyl-7H-pyrrolo[2,3-d]pyrimidine

To a solution of 4-chloro-5-cyclopropyl-7-tosyl-7H-pyrrolo[2,3-d]pyrimidine (2.4 g, 7.0 mmol, prepared following the procedure described for compound 164 in THE (15 mL) at 0° C. was added TBAF (42 mL, 1.0M in THF). The resulting mixture was stirred at 0° C. for 5 h. The reaction was quenched with water and extracted twice with EtOAc. The combined organic layers were washed with brine, dried over Na₂SO₄, filtered and concentrated. The residue was purified by flash column chromatography (silica gel, 0-10%, methanol in dichloromethane) to afford 4-chloro-5-cyclopropyl-7H-pyrrolo[2,3-d]pyrimidine (1.2 g, 90%) as a white solid. LC/MS ESI (m/z): 194 (M+H)⁺.

### Step 2. 4-Chloro-5-cyclopropyl-7-(3,5-difluorophenyl)-7H-pyrrolo[2,3-d]pyrimidine

To a solution of 4-chloro-5-cyclopropyl-7H-pyrrolo[2,3-d]pyrimidine (1.2 g, 6.2 mmol) in DCM (100 mL) were added (3,5-difluorophenyl)boronic acid (2.0 g, 12 mmol), pyridine (2.9 g, 37 mmol) and Cu(OAc)₂ (2.8 g, 16 mmol). The resulting mixture was stirred at room temperature under an O₂ atmosphere for 48 h. The reaction was diluted with DCM and filtered. The filtrate was washed with water and brine, dried over Na₂SO₄, filtered and concentrated. The residue was purified by flash column chromatography (silica gel, 0˜30%, ethyl acetate in petroleum ether) to afford 4-chloro-5-cyclopropyl-7-(3,5-difluorophenyl)-7H-pyrrolo[2,3-d]pyrimidine (1.5 g, 63%) as a solid. LC/MS ESI (m/z): 306 (M+H)⁺.

### Step 3. tert-Butyl (R)-4-(5-cyclopropyl-7-(3,5-difluorophenyl)-7H-pyrrolo[2,3-d]pyrimidin-4-yl)-2-ethylpiperazine-1-carboxylate

To a solution of 4-chloro-5-cyclopropyl-7-(3,5-difluorophenyl)-7H-pyrrolo[2,3-d]pyrimidine (70 mg, 0.23 mmol) in EtOH (5 mL) were added tert-butyl (R)-2-ethylpiperazine-1-carboxylate (98 mg, 0.46 mmol) and DIPEA (180 mg, 1.4 mmol). The resulting mixture was heated to 100° C. overnight. After cooling to room temperature, solvent was removed and the residue was purified by flash column chromatography (silica gel, 0˜30%, ethyl acetate in petroleum ether) to afford tert-butyl (R)-4-(5-cyclopropyl-7-(3,5-difluorophenyl)-7H-pyrrolo[2,3-d]pyrimidin-4-yl)-2-ethylpiperazine-1-carboxylate (45 mg, 41%) as a white solid. LC/MS ESI (m/z): 484 (M+H)⁺. ¹H NMR (400 MHz, CDCl3) δ 8.44 (s, 1H), 7.39-7.32 (m, 2H), 6.92 (s, 1H), 6.80-6.72 (m, 1H), 4.50 (d, J=13.0 Hz, 1H), 4.27-4.12 (m, 2H), 4.03 (d, J=11.6 Hz, 1H), 3.29 (d, J=10.0 Hz, 2H), 3.13-3.03 (m, 1H), 2.04-1.97 (m, 1H), 1.75-1.66 (m, 1H), 1.56-1.52 (m, 1H), 1.50 (s, 9H), 1.08-1.01 (m, 2H), 0.90-0.84 (m, 4H), 0.68-0.61 (m, 1H).

The following compounds were prepared by procedures analogous to the synthesis of compound 256 using the corresponding amines.

| Cmpd | | |
|---|---|---|
| No. | Chemical Name | LCMS and ¹H NMR |
| 257 | tert-Butyl (S)-4-(5-cyclopropyl- | LC/MS ESI (m/z): 484 (M + H)⁺. ¹H NMR (400 MHz, |
| | 7-(3,5-difluorophenyl)-7H- | CDCl3) δ 8.37 (s, 1H), 7.31-7.25 (m, 2H), 6.85 (s, 1H), |
| | pyrrolo[2,3-d]pyrimidin-4-yl)- | 6.73-6.66 (m, 1H), 4.42 (d, J = 12.4 Hz, 1H), 4.19-4.06 |
| | 2-ethylpiperazine-1-carboxylate | (m, 2H), 3.96 (d, J = 11.5 Hz, 1H), 3.27-3.17 (m, 2H), |
| | | 3.05-2.97 (m, 1H), 1.96-1.90 (m, 1H), 1.65-1.59 (m, |
| | | 1H), 1.48 (d, J = 6.8 Hz, 1H), 1.43 (s, 9H), 0.99-0.94 (m, |
| | | 2H), 0.83-0.77 (m, 4H), 0.61-0.55 (m, 1H). |
| 258 | tert-Butyl 7-(5-cyclopropyl-7- | LC/MS ESI (m/z): 482 (M + H)⁺. ¹H NMR (400 MHz, |
| | (3,5-difluorophenyl)-7H- | CDCl3) δ 8.41 (s, 1H), 7.37-7.32 (m, 2H), 6.90 (d, J = 0.7 |
| | pyrrolo[2,3-d]pyrimidin-4-yl)- | Hz, 1H), 6.79-6.72 (m, 1H), 3.85 (d, J = 4.2 Hz, 2H), 3.78- |
| | 4,7-diazaspiro[2.5]octane-4- | 3.73 (m, 2H), 3.61 (s, 2H), 2.02-1.97 (m, 1H), 1.50 (s, |
| | carboxylate | 9H), 1.06-0.99 (m, 4H), 0.86-0.80 (m, 2H), 0.77-0.73 |
| | | (m, 2H). |

### Example 61. Synthesis of tert-butyl (S)-4-(7-(4-cyanopyridin-2-yl)-5-cyclopropyl-7H-pyrrolo[2,3-d]pyrimidin-4-yl)-3-methylpiperazine-1-carboxylate (Compound 260)

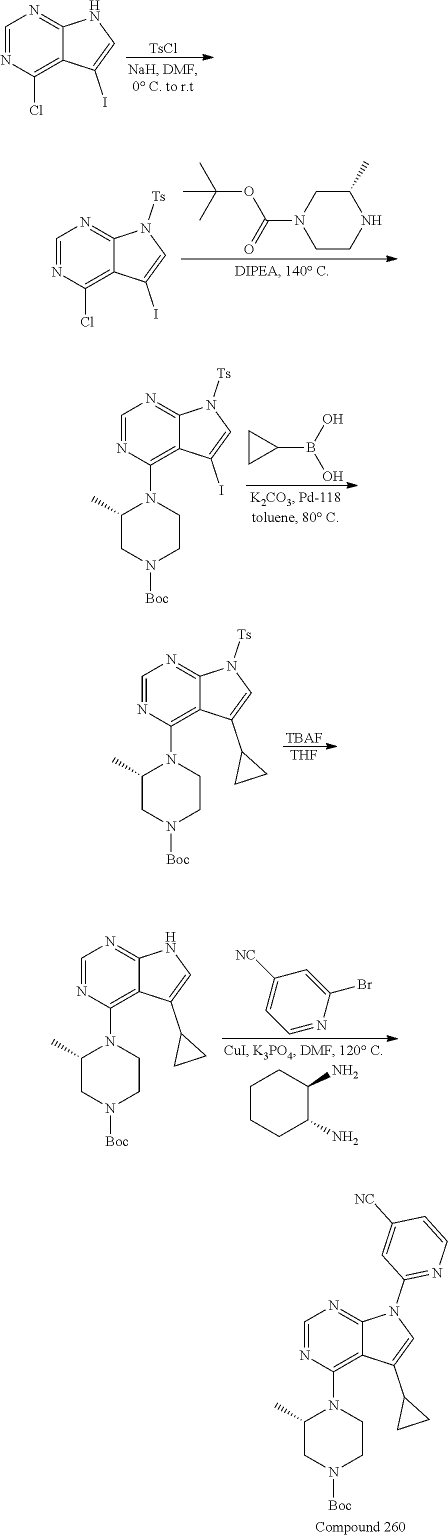

### Step 1. 4-Chloro-5-iodo-7-tosyl-7H-pyrrolo[2,3-d]pyrimidine

To a suspension of NaH (11 g, 270 mmol) in DMF (500 mL) at 0° C. was added a solution of 4-chloro-5-iodo-7H-pyrrolo[2,3-d]pyrimidine (50 g, 180 mmol) in DMF (150 m1) dropwise. After stirring at 0° C. for 10 minutes, a solution of TsCl (50 g, 260 mmol) in DMF (150 m1) was added dropwise. The resulting mixture was stirred at room temperature overnight. The reaction was poured into the ice water (4 L), filtered to give a crude product which was further dried in vacuo to afford 4-chloro-5-iodo-7-tosyl-7H-pyrrolo[2,3-d]pyrimidine (73 g, 94%) as a light yellow solid. LC/MS ESI (m/z): 434.2 (M+H)⁺.

### Step 2. tert-Butyl (S)-4-(5-iodo-7-tosyl-7H-pyrrolo[2,3-d]pyrimidin-4-yl)-3-methylpiperazine-1-carboxylate

A mixture of 4-chloro-5-iodo-7-tosyl-7H-pyrrolo[2,3-d]pyrimidine (50 g, 120 mmol) and tert-butyl (S)-3-methylpiperazine-1-carboxylate (46 g, 230 mmol) in DIPEA (350 m1) was heated to 140° C. for 1.5 h. DIPEA was removed and the residue was purified by flash chromatography (silica gel, 0˜30% ethyl acetate in petroleum ether) to afford tert-butyl(S)-4-(5-iodo-7-tosyl-7H-pyrrolo[2,3-d]pyrimidin-4-yl)-3-methylpiperazine-1-carboxylate (52 g, 75%) as a white solid. LC/MS ESI (m/z): 598.4 (M+H)⁺.

### Step 3. tert-Butyl (S)-4-(5-cyclopropyl-7-tosyl-7H-pyrrolo[2,3-d]pyrimidin-4-yl)-3-methylpiperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (39 g, 65 mmol) in toluene (400 mL) were added cyclopropylboronic acid (8.4 g, 98 mmol), K₂CO₃ (120 g, 850 mmol) and 1,1′-bis(di-tert-butylphosphino)ferrocene palladium dichloride (2.0 g, 3.2 mmol). The resulting mixture was heated to 80° C. overnight. After cooling to room temperature, the reaction mixture was filtered. The filtrate was partitioned between DCM and water. The aqueous layer was extracted twice with DCM. The combined organic layers were dried over Na₂SO₄, filtered and concentrated. The residue was purified by flash chromatography (0˜50% ethyl acetate in petroleum ether) to afford tert-butyl (S)-4-(5-cyclopropyl-7-tosyl-7H-pyrrolo[2,3-d]pyrimidin-4-yl)-3-methylpiperazine-1-carboxylate (22 g, 67% yield, >98% purity and 10 g, 80% purity) as a white solid. LC/MS ESI (m/z): 512.5 (M+H)⁺.

### Step 4. tert-Butyl (S)-4-(5-cyclopropyl-7H-pyrrolo[2,3-d]pyrimidin-4-yl)-3-methylpiperazine-1-carboxylate

To a solution of tert-butyl (S)-4-(5-cyclopropyl-7-tosyl-7H-pyrrolo[2,3-d]pyrimidin-4-yl)-3-methylpiperazine-1-carboxylate (79 g, 150 mmol) in THE (500 m1) was added TBAF (400 ml, 1.0M in THF). The resulting mixture was stirred at room temperature overnight. After removal of solvent, the residue was diluted with H₂O and extracted twice with DCM. The combined organic layers were dried over Na₂SO₄, filtered and concentrated. The residue was purified by flash chromatography (0-50% ethyl acetate in petroleum ether) to afford tert-butyl (S)-4-(5-cyclopropyl-7H-pyrrolo[2,3-d]pyrimidin-4-yl)-3-methylpiperazine-1-carboxylate (49 g, 88%) as light yellow solid. LC/MS ESI (m/z): 358.5 (M+H)⁺.

### Step 5. tert-Butyl (S)-4-(7-(4-cyanopyridin-2-yl)-5-cyclopropyl-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (18 g, 50 mmol) in DMF (180 mL) were added 2-bromoisonicotinonitrile (18 g, 100 mmol), CuI (4.8 g, 25 mmol), (+/−)-trans-1,2-diaminocyclohexane (1.7 g, 15 mmol) and K₃PO₄ (32 g, 150 mmol). The resulting mixture was heated to 120° C. overnight. After cooling to room temperature, the reaction was filtered, and solvent was removed. The residue was purified by flash chromatography (0˜30% EtOAc (with 50% DCM) in petroleum ether) to afford tert-butyl (S)-4-(7-(4-cyanopyridin-2-yl)-5-cyclopropyl-7H-pyrrolo[2,3-d]pyrimidin-4-yl)-3-methylpiperazine-1-carboxylate (12 g, 51% yield, >98% purity and 4.6 g, 90% purity) as a light yellow solid. LC/MS ESI (m/z): 460.6 (M+H)⁺. ¹H NMR (400 MHz, CDCl₃) δ 9.32 (s, 1H), 8.58 (d, J=5.0 Hz, 1H), 8.50 (s, 1H), 7.77 (s, 1H), 7.33 (dd, J=5.0, 1.2 Hz, 1H), 4.74-4.66 (m, 1H), 4.18-3.83 (m, 3H), 3.55 (t, J=11.6 Hz, 1H), 3.39-3.09 (m, 2H), 2.06-1.99 (m, 1H), 1.50 (s, 9H), 1.24 (d, J=6.5 Hz, 3H), 1.06-1.02 (m, 2H), 0.90-0.84 (m, 1H), 0.80-0.74 (m, 1H).

The following compounds were prepared by the procedure analogous to the synthesis of compound 260 from the corresponding amines, boronic acids and aryl halides. In the case of preparation of compounds 481 and 483, commercially available intermediate 4-chloro-5-(trifluoromethyl)-7H-pyrrolo[2,3-d]pyrimidine, was elaborated using the appropriate synthetic steps as outlined in the synthesis of compound 260.

| Cmpd | | |
|---|---|---|
| No. | Chemical Name | LCMS and ¹H NMR |
| 188 | tert-Butyl (S)-4-(5-cyclopropyl- | LC/MS ESI (m/z): 470 (M + H)⁺. 1H NMR (400 MHz, |
| | 7-(3,5-difluorophenyl)-7H- | CDCl3) δ 8.45 (s, 1H), 7.39-7.32 (m, 2H), 6.93 (d, J = 0.8 |
| | pyrrolo[2,3-d]pyrimidin-4-yl)-2- | Hz, 1H), 6.76 (tt, J = 8.8, 2.3 Hz, 1H), 4.52-4.28 (m, 2H), |
| | methylpiperazine-1-carboxylate | 4.10 (d, J = 13.0 Hz, 1H), 3.97 (d, J = 13.2 Hz, 1H), 3.48- |
| | | 3.25 (m, 2H), 3.09 (td, J = 12.4, 3.4 Hz, 1H), 2.13-1.97 |
| | | (m, 1H), 1.50 (s, 9H), 1.22 (d, J = 6.8 Hz, 3H), 1.09-0.99 |
| | | (m, 2H), 0.91-0.81 (m, 1H), 0.72-0.62 (m, 1H). |
| 476 | tert-butyl (S)-4-(7-(4- | LC/MS ESI (m/z): 434 (M + H)^{+.} ¹H NMR(400 MHz, |
| | cyanopyridin-2-yl)-5-methyl- | CDCl₃) δ 9.24 (s, 1H), 8.52 (d, J = 5.1 Hz, 1H), 8.45 (s, |
| | 7H-pyrrolo [2,3-d]pyrimidin-4- | 1H), 7.89 (s, 1H), 7.30-7.26 (m, 1H), 4.29-4.20 (m, 1H), |
| | yl)-3-methylpiperazine-1- | 4.08-3.87 (m, 1H), 3.82-3.67 (m, 1H), 3.63-3.55 (m, |
| | carboxylate | 1H), 3.49-3.38 (m, 1H), 3.30 (s, 1H), 3.13 (s, 1H), 2.38 (s, |
| | | 3H), 1.43 (s, 9H), 1.17 (d, J = 6.6 Hz, 3H). |
| 477 | tert-butyl (2R,5S)-4-(7-(4- | LC/MS ESI (m/z): 448 (M + H)^{+.} ¹H NMR(400 MHz, |
| | cyanopyridin-2-yl)-5-methyl- | MeOD) δ 9.21 (s, 1H), 8.66 (d, J = 4.8 Hz, 1H), 8.45 (s, |
| | 7H-pyrrolo[2,3-d]pyrimidin-4- | 1H), 8.04 (s, 1H), 7.54 (d, J = 3.9 Hz, 1H), 4.56-4.50 (m, |
| | yl)-2,5-dimethylpiperazine-1- | 1H), 4.42-4.35 (m, 1H), 3.82-3.76 (m, 2H), 3.68-3.61 |
| | carboxylate | (m, 1H), 3.57-3.52 (m, 1H), 2.49 (s, 3H), 1.50 (s, 9H), |
| | | 1.18-1.14 (m, 6H). |
| 478 | tert-butyl (S)-4-(7-(4- | LC/MS ESI (m/z): 448 (M + H)^{+.} ¹H NMR (400 MHz, |
| | cyanopyridin-2-yl)-5-ethyl-7H- | CDCl₃) δ 9.26 (s, 1H), 8.53 (d, J = 4.9 Hz, 1H), 8.47 (s, |
| | pyrrolo[2,3-d]pyrimidin-4-yl)-3- | 1H), 7.91 (s, 1H), 7.28 (d, J = 4.6 Hz, 1H), 4.17 (br s, 1H), |
| | methylpiperazine-1-carboxylate | 3.94 (br s, 1H), 3.67 (br s, 1H), 3.47 (br s, 2H), 3.36 (br s, |
| | | 1H), 3.16 (br s, 1H), 2.81-2.73 (m, 2H), 1.43 (s, 9H), 1.29 |
| | | (t, J = 7.4 Hz, 3H), 1.14 (d, J = 6.4 Hz, 3H). |
| 479 | tert-butyl (2R,5S)-4-(7-(4- | LC/MS ESI (m/z): 462 (M + H)^{+.} ¹H NMR(400 MHz, |
| | cyanopyridin-2-yl)-5-ethyl-7H- | CDCl₃) δ 9.33 (s, 1H), 8.60 (d, J = 4.6 Hz, 1H), 8.52 (s, |
| | pyrrolo[2,3-d]pyrimidin-4-yl)- | 1H), 7.98 (s, 1H), 7.35 (d, J = 5.0 Hz, 1H), 4.52-4.35 (m, |
| | 2,5-dimethylpiperazine-1- | 2H), 3.81-3.74 (m, 2H), 3.67-3.60 (m, 1H), 3.49-3.43 |
| | carboxylate | (m, 1H), 2.85 (q, J = 7.3 Hz, 2H), 1.50 (s, 9H), 1.36 (t, J = |
| | | 7.4 Hz, 3H), 1.17-1.13 (m, 6H). |
| 480 | tert-butyl (2R,5S)-4-(7-(4- | LC/MS ESI (m/z): 441 (M + H)^{+.} ¹H NMR (400 MHz, |
| | fluoropyridin-2-yl)-5-methyl- | CDCl₃) δ 8.64 (dd, J = 11.1 Hz, 1H), 8.42 (s, 1H), 8.34 (t, |
| | 7H-pyrrolo[2,3-d]pyrimidin-4- | J = 8.6, 5.7 Hz, 1H), 7.90 (s, 1H), 6.87-6.81 (m, 1H), 4.45- |
| | yl)-2,5-dimethylpiperazine-1- | 4.27 (m, 2H), 3.74-3.64 (m, 2H), 3.58-3.50 (m, 1H), |
| | carboxylate | 3.42 (d, J = 13.1 Hz, 1H), 2.38 (s, 3H), 1.43 (s, 9H), 1.11- |
| | | 1.05 (m, 6H). |
| 481 | tert-butyl (S)-4-(7-(4- | LC/MS ESI (m/z): 488 (M + H)^{+.} ¹H NMR (400 MHz, |
| | cyanopyridin-2-yl)-5- | CDCl₃) δ 9.34 (s, 1H), 8.69-8.64 (m, 2H), 8.62 (s, 1H), |
| | (trifluoromethyl)-7H- | 7.49 (d, J = 4.9 Hz, 1H), 4.41-4.35 (m, 1H), 4.07-3.90 |
| | pyrrolo[2,3-d]pyrimidin-4-yl)-3- | (m, 1H), 3.73 (d, J = 12.2 Hz, 1H), 3.59-3.40 (m, 3H), |
| | methylpiperazine-1-carboxylate | 3.28-3.12 (m, 1H), 1.50 (s, 9H), 1.21 (d, J = 6.5 Hz, 3H). |
| 483 | tert-butyl (2R,5S)-4-(7-(4- | LC/MS ESI (m/z): 502 (M + H)^{+.} ¹H NMR (400 MHz, |
| | cyanopyridin-2-yl)-5- | CDCl₃) δ 9.27 (s, 1H), 8.60 (d, J = 4.9 Hz, 1H), 8.57 (s, |
| | (trifluoromethyl)-7H- | 1H), 8.50 (s, 1H), 7.41 (d, J = 4.9 Hz, 1H), 4.52 (s, 1H), |
| | pyrrolo[2,3-d]pyrimidin-4-yl)- | 4.31 (s, 1H), 3.79-3.63 (m, 2H), 3.61-3.52 (m, 1H), 3.41 |
| | 2,5-dimethylpiperazine-1- | (d, J = 13.3 Hz, 1H), 1.42 (s, 9H), 1.09 (d, J = 6.6 Hz, 3H), |
| | carboxylate | 1.03 (d, J = 6.7 Hz, 3H). |

### Example 62. Synthesis of tert-butyl (S)-3-methyl-4-(1-phenyl-3-(tetrahydro-2H-pyran-4-yl)-1H-pyrazolo[3,4-d]pyrimidin-4-yl)piperazine-1-carboxylate (Compound 261)

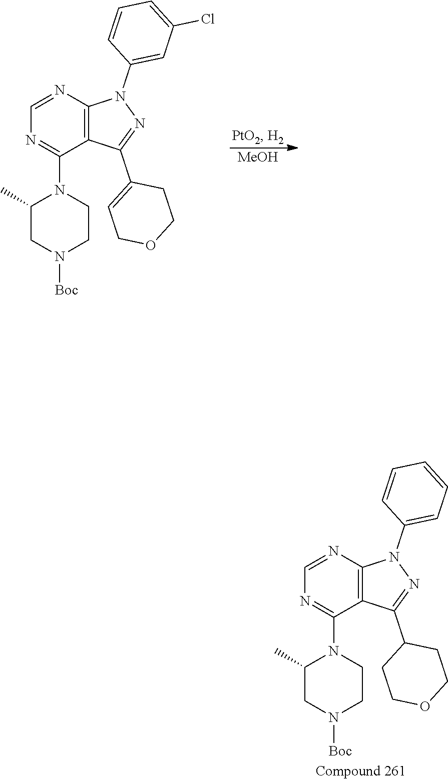

A mixture of tert-butyl (S)-4-(1-(3-chlorophenyl)-3-(3,6-dihydro-2H-pyran-4-yl)-1H-pyrazolo[3,4-d]pyrimidin-4-yl)-3-methylpiperazine-1-carboxylate (170 mg, 0.33 mmol, prepared following the procedures described for compound 284), PtO₂ (100 mg) in MeOH (8 mL) was stirred at room temperature for overnight under a H₂ atmosphere. The reaction was filtered, the MeOH was removed by vacuum, the residue was purified by flash column chromatography (silica gel, 0˜50% EtOAc in petroleum ether) and prep-HPLC to give tert-butyl (S)-3-methyl-4-(1-phenyl-3-(tetrahydro-2H-pyran-4-yl)-1H-pyrazolo[3,4-d]pyrimidin-4-yl)piperazine-1-carboxylate (50 mg, 32% yield) as a white solid LCMS ESI (m/z): 479. (M+H)⁺. ¹H NMR (400 MHz, CDCl₃) δ 8.51 (s, 1H), 8.18-8.11 (m, 2H), 7.52 (dd, J=10.8, 5.2 Hz, 2H), 7.35-7.28 (m, 1H), 4.56-4.41 (m, 1H), 4.19-3.80 (m, 5H), 3.63-3.50 (m, 3H), 3.33-3.03 (m, 3H), 2.25-2.13 (m, 1H), 2.09-1.98 (m, 3H), 1.50 (s, 9H), 1.31 (d, J=6.7 Hz, 3H).

### Example 63. Synthesis of tert-butyl (S)-4-(7-(4-cyanopyridin-2-yl)-5-(2-fluorophenyl)-7H-pyrrolo[2,3-d]pyrimidin-4-yl)-3-methylpiperazine-1-carboxylate (Compound 268)

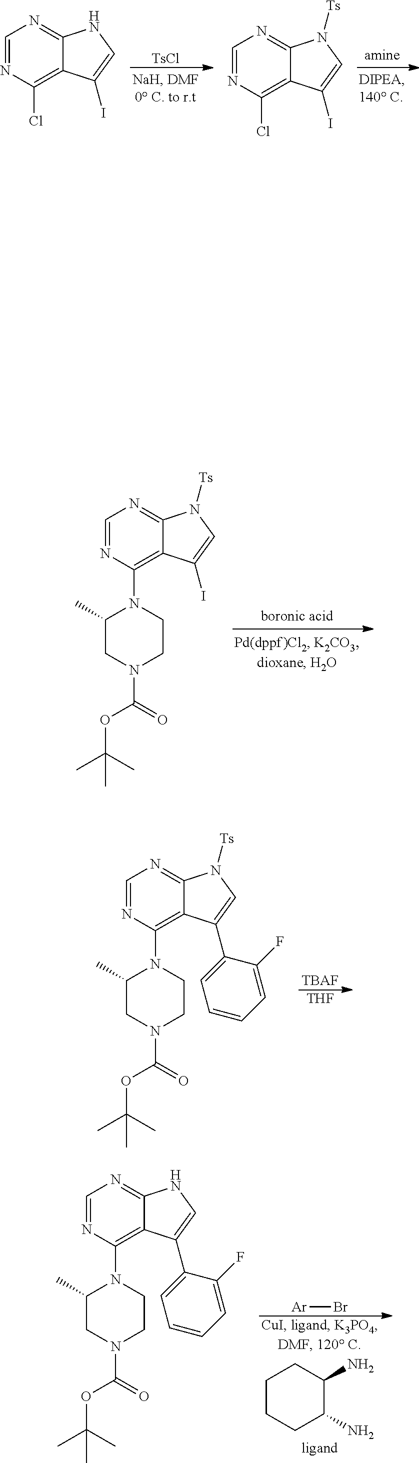

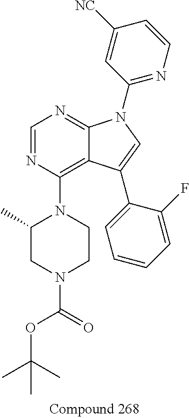 [SMILES: C[C@H]1CN(C(=O)OC(C)(C)C)CCN1c1ncnc2c1c(-c1ccccc1F)cn2-c1cc(C#N)ccn1]

### Step 1. 4-Chloro-5-iodo-7-tosyl-7H-pyrrolo[2,3-d]pyrimidine

At 0° C., to a suspension of NaH (11 g, 270 mmol) in DMF (500 mL) was added solution of 4-chloro-5-iodo-7H-pyrrolo[2,3-d]pyrimidine (50 g, 180 mmol) in DMF (150 m1). After being stirred at 0° C. for 10 minutes, a solution of TsCl (50 g, 260 mmol) in DMF (150 m1) was added dropwise into the reaction mixture. The resulting mixture was stirred at room temperature overnight. The reaction was poured into ice water (4 L), filtered and concentrated. 4-chloro-5-iodo-7-tosyl-7H-pyrrolo[2,3-d]pyrimidine (67 g, 86%). as a light yellow solid. LC/MS ESI (m/z): 434.2 (M+H)⁺.

### Step 2. tert-Butyl (S)-4-(5-iodo-7-tosyl-7H-pyrrolo[2,3-d]pyrimidin-4-yl)-3-methylpiperazine-1-carboxylate

The mixture of 4-chloro-5-iodo-7-tosyl-7H-pyrrolo[2,3-d]pyrimidine (50 g, 120 mmol) and tert-butyl (S)-3-methylpiperazine-1-carboxylate (46 g, 230 mmol) in DIEA (350 m1) was heated to 140° C. for 1.5 h. the reaction mixture was purified by flash chromatography (silica gel, 0˜30% ethyl acetate in petroleum ether) to afford tert-butyl (S)-4-(5-iodo-7-tosyl-7H-pyrrolo[2,3-d]pyrimidin-4-yl)-3-methylpiperazine-1-carboxylate (52 g, 75%) as a white solid. LC/MS ESI (m/z): 598.4 (M+H)⁺.

### Step 3. tert-Butyl (S)-4-(5-(2-fluorophenyl)-7-tosyl-7H-pyrrolo[2,3-d]pyrimidin-4-yl)-3-methylpiperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (52 g, 87 mmol) in dioxane-water (500 mL; v:v=5:1) was added (2-fluorophenyl)boronic acid (24 g, 170 mmol), K₂CO₃ (36 g, 260 mmol) and Pd(dppf)Cl₂ (3.5 g, 4.3 mmol). The resulting mixture was heated to 80° C. overnight. After being cooled down to room temperature, the reaction mixture was filtered. The filtrate was partitioned between DCM and water, organic layer was separated, aqueous layer was extracted with DCM twice, the combined organic layers were dried over Na₂SO₄, filtered and concentrated. The residue was purified by flash chromatography (0-50% ethyl acetate in petroleum ether) to afford tert-butyl (S)-4-(5-(2-fluorophenyl)-7-tosyl-7H-pyrrolo[2,3-d]pyrimidin-4-yl)-3-methylpiperazine-1-carboxylate (34 g, 70%) as a white solid. LC/MS ESI (m/z): 566.6 (M+H)⁺.

### Step 4. tert-Butyl (S)-4-(5-(2-fluorophenyl)-7H-pyrrolo[2,3-d]pyrimidin-4-yl)-3-methylpiperazine-1-carboxylate

To a solution of tert-butyl (3S)-4-[5-(2-fluorophenyl)-7-(4-methylbenzenesulfonyl)-7H-pyrrolo[2,3-d]pyrimidin-4-yl]-3-methylpiperazine-1-carboxylate (37 g, 65 mmol) in THE (300 m1) was added TBAF (220 ml, 1.0M in THF). The resulting mixture was stirred at room temperature overnight. After removed 2/3 solvent, the reaction mixture was poured into ice water, the precipitate was filtered and dried in vacuo to give tert-butyl (S)-4-(5-(2-fluorophenyl)-7H-pyrrolo[2,3-d]pyrimidin-4-yl)-3-methylpiperazine-1-carboxylate (25 g, 93%) as a gray solid. LC/MS ESI (m/z): 412.5 (M+H)⁺.

### Step 5. tert-Butyl (S)-4-(7-(4-cyanopyridin-2-yl)-5-(2-fluorophenyl)-7H-pyrrolo[2,3-d]pyrimidin-4-yl)-3-methylpiperazine-1-carboxylate

To a solution of tert-butyl (S)-4-(5-(2-fluorophenyl)-7H-pyrrolo[2,3-d]pyrimidin-4-yl)-3-methylpiperazine-1-carboxylate (10 g, 24 mmol) in DMF (80 mL) were added 2-bromopyridine-4-carbonitrile (8.9 g, 49 mmol), CuI (1.4 g, 7.3 mmol), trans-cyclohexane-1,2-diamine (0.83 g, 7.3 mmol) and K₃PO₄ (16 g, 73 mmol). The resulting mixture was heated to 120° C. overnight under N₂. After being cooled down to room temperature, the reaction was filtered, and solvent was removed. The residue was purified by flash chromatography (ethyl acetate: petroleum ether:dichloromethane=5:1:1) to afford tert-butyl (S)-4-(7-(4-cyanopyridin-2-yl)-5-(2-fluorophenyl)-7H-pyrrolo[2,3-d]pyrimidin-4-yl)-3-methylpiperazine-1-carboxylate (9.3 g, 75%) as an off-white solid. LC/MS ESI (m/z): 514.6 (M+H)⁺. ¹H NMR (400 MHz, CDCl3) δ 9.40 (s, 1H), 8.61 (d, J=4.9 Hz, 1H), 8.56 (s, 1H), 8.27 (s, 1H), 7.46 (t, J=7.2 Hz, 1H), 7.41-7.35 (m, 2H), 7.27-7.18 (m, 2H), 4.24 (s, 1H), 3.77 (s, 1H), 3.56-3.44 (m, 2H), 3.08 (t, J=11.5 Hz, 1H), 2.89-2.62 (m, 2H), 1.43 (s, 9H), 1.02 (d, 3H).

The following compounds were prepared by the procedures similar to the synthesis of compound 268 from the amines and aryl halides.

| Cmpd | | |
|---|---|---|
| No. | Chemical Name | LCMS and ¹H NMR |
| 452 | tert-butyl (2R,5S)-4-(5-(2- | LC/MS (ESI) (m/z): 521 (M + H)⁺. ¹H NMR (400 MHz, |
| | fluorophenyl)-7-(4- | CDCl₃) δ 8.81 (dd, J = 10.9, 1.8 Hz, 1H), 8.55 (s, 1H), 8.42 |
| | fluoropyridin-2-yl)-7H- | (dd, J = 8.5, 5.7 Hz, 1H), 8.29 (s, 1H), 7.48 (t, J = 7.5 Hz, |
| | pyrrolo[2,3-d]pyrimidin-4-yl)- | 1H), 7.36 (dd, J = 12.8, 6.5 Hz, 1H), 7.20 (dd, J = 17.2, 8.2 |
| | 2,5-dimethylpiperazine-1- | Hz, 2H), 6.94 (dd, J = 9.3, 3.8 Hz, 1H), 4.29-4.03 (m, |
| | carboxylate | 2H), 3.40-3.18 (m, 3H), 2.99-2.72 (m, 1H), 1.42 (s, 9H), |
| | | 1.06 (d, J = 6.8 Hz, 3H), 0.95 (d, J = 6.6 Hz, 3H). |
| 212 | tert-butyl (S)-4-(7-(3-chloro-4- | LC/MS(ESI)m/z: 523(M + H)⁺ ¹H NMR (400 MHz, CDCl₃) |
| | fluorophenyl)-5-(pyridin-3-yl)- | 8 8.82 (s, 1H), 8.62 (s, 1H), 8.53 (s, 1H), 7.88-7.78 (m, |
| | 7H-pyrrolo[2,3-d]pyrimidin-4- | 2H), 7.64 (ddd, J = 8.8, 4.1, 2.7 Hz, 1H), 7.41 (dd, J = 7.5, |
| | yl)-3-methylpiperazine-1- | 5.0 Hz, 1H), 7.32 (dd, J = 11.3, 6.1 Hz, 2H), 4.12 (d, J = |
| | carboxylate | 6.3 Hz, 1H), 3.92-3.69 (m, 1H), 3.49 (dd, J = 30.3, 11.0 |
| | | Hz, 2H), 3.14 (td, J = 12.9, 3.2 Hz, 1H), 2.99-2.67 (m, |
| | | 2H), 1.42 (s, 9H), 1.01 (d, J = 5.7 Hz, 3H). |
| 213 | tert-butyl-(S)-4-(7-(3,5- | LC/MS ESI (m/z): 507 (M + H)⁺. ¹H NMR (400 MHz, |
| | difluorophenyl)-5-(pyridin-3-yl)- | CDCl₃) δ 8.83 (s, 1H), 8.63 (d, J = 3.3 Hz, 1H), 8.56 (s, |
| | 7H-pyrrolo[2,3-d]pyrimidin-4- | 1H), 7.87 (d, J = 7.7 Hz, 1H), 7.49-7.35 (m, 4H), 6.84 (tt, |
| | yl)-3-methylpiperazine-1- | J = 8.7, 2.3 Hz, 1H), 4.12 (d, J = 5.5 Hz, 1H), 3.80 (dd, J = |
| | carboxylate | 40.7, 14.9 Hz, 1H), 3.49 (dd, J = 36.2, 12.3 Hz, 2H), 3.21- |
| | | 3.06 (m, 1H), 3.01-2.65 (m, 2H), 1.42 (s, 9H), 1.02 (d, J = |
| | | 5.4 Hz, 3H). |
| 272 | tert-butyl-(S)-4-(7-(3- | LC/MS ESI (m/z): 501 (M + H)⁺. ¹H NMR (400 MHz, |
| | methoxyphenyl)-5-(pyridin-3- | CDCl₃) δ 8.85 (s, 1H), 8.62 (s, 1H), 8.56 (s, 1H), 7.90 (d, |
| | yl)-7H-pyrrolo[2,3-d]pyrimidin- | J = 7.7 Hz, 1H), 7.44 (dd, J = 16.6, 8.4 Hz, 3H), 7.32-7.27 |
| | 4-yl)-3-methylpiperazine-1- | (m, 2H), 7.01-6.91 (m, 1H), 4.09 (dd, J = 13.0, 9.6 Hz, |
| | carboxylate | 1H), 3.88 (s, 3H), 3.84-3.67 (m, 1H), 3.60-3.36 (m, 2H), |
| | | 3.22-3.09 (m, 1H), 3.03-2.70 (m, 2H), 1.43 (s, 9H), 1.02 |
| | | (d, J = 6.0 Hz, 3H). |
| 281 | tert-butyl-(S)-3-methyl-4-(5- | LC/MS ESI (m/z): 555 (M + H)⁺. ¹H NMR (400 MHz, |
| | (pyridin-3-yl)-7-(3- | CDCl₃) δ 8.85 (s, 1H), 8.64 (s, 1H), 8.56 (s, 1H), 7.89 (d, |
| | (trifluoromethoxy)phenyl)-7H- | J = 7.5 Hz, 1H), 7.74 (d, J = 8.1 Hz, 1H), 7.68 (s, 1H), 7.58 |
| | pyrrolo [2,3-d]pyrimidin-4- | (t, J = 8.2 Hz, 1H), 7.42 (d, J = 15.5 Hz, 2H), 7.29-7.26 |
| | yl)piperazine-1-carboxylate | (m, 1H), 4.12 (s, 1H), 3.81 (dd, J = 55.7, 17.6 Hz, 1H), 3.49 |
| | | (dd, J = 31.8, 11.1 Hz, 2H), 3.23-3.07 (m, 1H), 2.99- |
| | | 2.71 (m, 2H), 1.43 (s, 9H), 1.02 (d, J = 5.6 Hz, 3H). |
| 282 | tert-butyl-(S)-4-(7-(3- | LC/MS ESI (m/z): 496 (M + H)⁺. ¹H NMR (400 MHz, |
| | cyanophenyl)-5-(pyridin-3-yl)- | CDCl₃) δ 8.84 (s, 1H), 8.64 (s, 1H), 8.54 (s, 1H), 8.15 (s, |
| | 7H-pyrrolo[2,3-d]pyrimidin-4- | 1H), 8.05 (dd, J = 5.9, 4.5 Hz, 1H), 7.88 (d, J = 7.6 Hz, |
| | yl)-3-methylpiperazine-1- | 1H), 7.67 (d, J = 5.0 Hz, 2H), 7.48-7.36 (m, 2H), 4.15 (s, |
| | carboxylate | 1H), 3.81 (dd, J = 54.1, 18.7 Hz, 1H), 3.60-3.41 (m, 2H), |
| | | 3.20-3.08 (m, 1H), 3.01-2.69 (m, 2H), 1.42 (s, 9H), 1.02 |
| | | (d, J = 4.9 Hz, 3H). |
| 170 | tert-butyl (S)-3-methyl-4-(7-(1- | LC/MS ESI (m/z): 474 (M + H)⁺. ¹H NMR (400 MHz, |
| | methyl-1H-pyrazol-4-yl)-5- | CDCl₃) δ 8.50 (s, 1H), 8.10 (s, 1H), 7.77 (s, 1H), 7.50 (d, |
| | phenyl-7H-pyrrolo[2,3- | J = 7.2 Hz, 2H), 7.44 (dd, J = 10.4, 4.8 Hz, 2H), 7.37-7.31 |
| | d]pyrimidin-4-yl)piperazine-1- | (m, 1H), 7.24 (s, 1H), 4.16 (t, J = 10.3 Hz, 1H), 3.99 (s, |
| | carboxylate | 3H), 3.90-3.71 (m, 1H), 3.51 (d, J = 13.1 Hz, 2H), 3.11 |
| | | (td, J = 13.0, 3.3 Hz, 1H), 2.92-2.62 (m, 2H), 1.42 (s, 9H), |
| | | 0.97 (d, J = 6.3 Hz, 3H). |
| 177 | tert-butyl (S)-3-methyl-4-(7-(5- | LC/MS(ESI)m/z: 486 (M + H)⁺ |
| | methylpyridin-3-yl)-5-(pyridin- | ¹H NMR (400 MHz, CDCl₃) δ 8.79 (d, J = 30.4 Hz, 2H), |
| | 3-yl)-7H-pyrrolo[2,3- | 8.63 (s, 1H), 8.54 (s, 1H), 8.48 (s, 1H), 8.01 (s, 1H), 7.87 |
| | d]pyrimidin-4-yl)piperazine-1- | (d, J = 7.8 Hz, 1H), 7.47-7.35 (m, 2H), 4.13 (s, 1H), 3.92- |
| | carboxylate | 3.69 (m, 1H), 3.50 (dd, J = 27.8, 11.6 Hz, 2H), 3.20- |
| | | 3.09 (m, 1H), 2.99-2.69 (m, 2H), 2.48 (s, 3H), 1.42 (s, |
| | | 9H), 1.02 (d, J = 6.1 Hz, 3H). |
| 320 | tert-butyl (S)-4-(7-(4- | LC/MS ESI (m/z): 496 (M + H)⁺. ¹H NMR (400 MHz, |
| | cyanopyridin-2-yl)-5-phenyl- | CDCl₃) δ 9.40 (s, 1H), 8.62 (d, J = 5.0 Hz, 1H), 8.59 (s, |
| | 7H-pyrrolo[2,3-d]pyrimidin-4- | 1H), 8.21 (s, 1H), 7.53 (d, J = 7.3 Hz, 2H), 7.46 (t, J = 7.5 |
| | yl)-3-methylpiperazine-1- | Hz, 2H), 7.41-7.35 (m, 2H), 4.24 (s, 1H), 3.88-3.73 (m, |
| | carboxylate | 1H), 3.56-3.43 (m, 2H), 3.16-3.04 (m, 1H), 2.95-2.64 |
| | | (m, 2H), 1.42 (s, 9H), 1.01 (s, 3H). |
| 105 | tert-butyl (S)-4-(5-(2- | LC/MS ESI (m/z): 519 (M + H)⁺. ¹H NMR (400 MHz, |
| | fluorophenyl)-7-(5- | CDCl3) 88.54 (s, 1H) , 8.50 (s, 1H) , 8.34 (d, J = 2.4 Hz, |
| | methoxypyridin-3-yl)-7H- | 1H) , 7.82 (t, J = 2.3 Hz, 1H) , 7.53-7.46 (m, 1H) , 7.44 (s, |
| | pyrrolo[2, 3-d]pyrimidin-4-yl)- | 1H) , 7.43-7.33 (m, 1H) , 7.29 (d, -7.17 (m, 2H) , 4.25 |
| | 3-methylpiperazine-1- | (s, 1H) , 3.96 (s, 3H) , 3.85 (d, J = 13.0Hz, 1H) , 3.66 (d, J = |
| | carboxylate | 12.0Hz, 1H) , 3.45 (d, J = 13.1Hz, 1H) , 3.02 (dd, J = 13.1, |
| | | 3.9Hz, 1H) , 2.78-2.68 (m, 1H) , 2.60 (t, J = 11.1 Hz, 1H), |
| | | 1.43 (s, 9H) , 1.13 (d, J = 6.8Hz, 3H). |
| 444 | tert-butyl (S)-4-(7-(2-cyano-6- | LC/MS ESI (m/z): 528 (M + H)+. ¹H NMR (400 MHz, |
| | methylpyridin-4-yl)-5-(2- | MeOD) δ 8.64 (d, J = 1.6 Hz, 1H), 8.50 (s, 1H), 8.27 (d, J = |
| | fluorophenyl)-7H-pyrrolo[2,3- | 1.6 Hz, 1H), 7.95 (s, 1H), 7.58-7.52 (m, 1H), 7.51-7.45 |
| | d]pyrimidin-4-yl)-3- | (m, 1H), 7.37-7.26 (m, 2H), 4.17 (s, 1H), 3.71 (d, J = 12.8 |
| | methylpiperazine-1-carboxylate | Hz, 1H), 3.50 (d, J = 13.0 Hz, 2H), 3.13-3.05 (m, 1H), |
| | | 2.67 (s, 2H), 2.66 (s, 3H), 1.42 (s, 9H), 0.97 (d, J = 6.4 Hz, 3H) |
| 434 | tert-butyl (2R,5S)-4-(7-(5- | LC/MS ESI (m/z): 534 (M + H)+. ¹H NMR (400 MHz, |
| | cyanothiazol-2-yl)-5-(2- | CDCl₃) δ 8.60 (s, 1H), 8.08 (s, 2H), 7.49-7.40 (m, 2H), |
| | fluorophenyl)-7H-pyrrolo[2,3- | 7.29 (s, 1H), 7.25-7.18 (m, 1H), 4.38-4.06 (m, 2H), 3.44- |
| | d]pyrimidin-4-yl)-2,5- | 3.31 (m, 2H), 3.26-3.17 (m, 1H), 3.10-2.87 (m, 1H), |
| | dimethylpiperazine-1- | 1.43 (s, 9H), 1.02 (d, J = 6.4 Hz, 6H). 1⁹F NMR (377 MHz, |
| | carboxylate | CDCl₃) δ −114.30 (s). |
| 455 | tert-butyl 4-(7-(5-cyanothiazol- | LC/MS ESI (m/z): 506 (M + H)+. ¹H NMR (400 MHz, |
| | 2-yl)-5-(2-fluorophenyl)-7H- | CDCl₃) δ 8.53 (s, 1H), 8.02 (d, J = 7.8 Hz, 2H), 7.41-7.32 |
| | pyrrolo[2,3-d]pyrimidin-4- | (m, 2H), 7.24-7.19 (m, 1H), 7.17-7.12 (m, 1H), 3.30- |
| | yl)piperazine-1-carboxylate | 3.22 (m, 4H), 3.19-3.12 (m, 4H), 1.36 (s, 9H). ¹⁹F NMR |
| | | (377 MHz, CDCl₃) δ −114.61 (s). |
| 461 | tert-butyl (2R,5S)-4-(7-(3- | LC/MS ESI (m/z): 534 (M + H)+. ¹H NMR (400 MHz, |
| | chlorophenyl)-5-(3- | CDCl₃) δ 8.53 (d, J = 2.8 Hz, 3H), 7.81 (s, 1H), 7.69 (d, J = |
| | methylpyrazin-2-yl)-7H- | 8.0 Hz, 1H), 7.51-7.46 (m, 2H), 7.38 (d, J = 8.1 Hz, 1H), |
| | pyrrolo[2,3-d]pyrimidin-4-yl)- | 4.27-4.01 (m, 2H), 3.38-3.27 (m, 2H), 3.18-3.05 (m, |
| | 2,5-dimethylpiperazine-1- | 1H), 2.94-2.69 (m, 1H), 2.54 (s, 3H), 1.43 (s, 9H), 1.00 (d, |
| | carboxylate | J = 6.4 Hz, 6H). |
| 131 | tert-butyl (2R,5S)-4-(7-(3- | LC/MS ESI (m/z): 519 (M + H)+. ¹H NMR (400 MHz, |
| | chlorophenyl)-5-(pyridin-3-yl)- | CDCl3) δ 8.85 (d, J = 1.6 Hz, 1H), 8.64 (d, J = 3.6 Hz, 1H), |
| | 7H-pyrrolo[2,3-d]pyrimidin-4- | 8.56 (s, 1H), 7.91-7.86 (m, 1H), 7.83-7.75 (m, 1H), 7.71- |
| | yl)-2,5-dimethylpiperazine-1- | 7.65 (m, 1H), 7.54-7.46 (m, 1H), 7.44-7.37 (m, 3H), |
| | carboxylate | 4.37-4.04 (m, 2H), 3.38-3.26 (m, 3H), 3.07-2.84 (m, |
| | | 1H), 1.43 (s, 9H), 1.13 (d, J = 6.8 Hz, 3H), 0.98 (d, J = 6.7 |
| | | Hz, 3H). |
| 130 | tert-butyl (2R,5S)-4-(7-(3- | LC/MS ESI (m/z): 482 (M + H)+. ¹H NMR (400 MHz, |
| | chlorophenyl)-5-cyclopropyl- | CDCl3) δ 8.43 (s, 1H), 7.69-7.66 (m, 1H), 7.61 (dd, J = |
| | 7H-pyrrolo[2,3-d]pyrimidin-4- | 8.1, 1.0 Hz, 1H), 7.45-7.40 (m, 1H), 7.31 (dd, J = 8.1, 0.9 |
| | yl)-2,5-dimethylpiperazine-1- | Hz, 1H), 6.93 (s, 1H), 4.96-4.87 (m, 1H), 4.55-4.29 (m, |
| | carboxylate | 1H), 3.81-3.70 (m, 3H), 3.63-3.53 (m, 1H), 2.06-1.99 |
| | | (m, 1H), 1.50 (s, 9H), 1.22-1.18 (m, 3H), 1.17-1.13 (m, |
| | | 3H), 1.05-1.00 (m, 2H), 0.89-0.85 (m, 1H), 0.68-0.62 |
| | | (m, 1H). |
| 231 | tert-butyl (S)-4-(5-(2- | LC/MS ESI (m/z): 489.4 (M + H)⁺. ¹H NMR (400 MHz, |
| | fluorophenyl)-7-(pyridin-2-yl)- | CDCl₃) δ 8.78 (d, J = 8.3 Hz, 1H), 8.56 (s, 1H), 8.50 (dd, |
| | 7H-pyrrolo[2,3-d]pyrimidin-4- | J = 4.8, 1.1 Hz, 1H), 8.25 (s, 1H), 7.90 (td, J = 8.4, 1.9 Hz, |
| | yl)-3-methylpiperazine-1- | 1H), 7.48 (td, J = 7.6, 1.6 Hz, 1H), 7.39-7.31 (m, 1H), |
| | carboxylate | 7.25-7.15 (m, 3H), 4.29-4.05 (m, 1H), 3.93-3.60 (m, |
| | | 1H), 3.52-3.45 (m, 2H), 3.15-3.03 (m, 1H), 2.90-2.59 |
| | | (m, 2H), 1.43 (s, 9H), 1.00 (d, J = 6.4 Hz, 3H). |
| 428 | tert-butyl (S)-4-(7-(5- | LC/MS ESI (m/z): 520.5 (M + H)⁺. ¹H NMR (400 MHz, |
| | cyanothiazol-2-yl)-5-(2- | CDCl₃) δ 8.58 (s, 1H), 8.11-8.03 (m, 2H), 7.49-7.37 (m, |
| | fluorophenyl)-7H-pyrrolo[2,3- | 2H), 7.30-7.27 (m, 1H), 7.24-7.19 (m, 1H), 4.46-4.21 |
| | d]pyrimidin-4-yl)-3- | (m, 1H), 3.88-3.69 (m, 1H), 3.63-3.47 (m, 2H), 3.13- |
| | methylpiperazine-1-carboxylate | 3.02 (m, 1H), 2.94-2.55 (m, 2H), 1.43 (s, 9H), 1.08-1.00 |
| | | (m, 3H). |
| 433 | tert-butyl (S)-4-(7-(4- | LC/MS ESI (m/z): 520 (M + H)+. ¹H NMR (400 MHz, |
| | cyanothiazol-2-yl)-5-(2- | CDCl₃) δ 8.50 (s, 1H), 8.01 (s, 1H), 7.80 (s, 1H), 7.41- |
| | fluorophenyl)-7H-pyrrolo[2,3- | 7.31 (m, 2H), 7.23-7.10 (m, 2H), 4.34-4.10 (m, 1H), |
| | d]pyrimidin-4-yl)-3- | 3.83-3.59 (m, 1H), 3.48 (dd, J = 27.9, 13.0 Hz, 2H), 3.09- |
| | methylpiperazine-1-carboxylate | 2.90 (m, 1H), 2.88-2.45 (m, 2H), 1.36 (s, 9H), 0.96 (d, |
| | | J = 5.6 Hz, 3H). |
| 178 | tert-butyl (2R,5S)-2,5-dimethyl- | LC/MS ESI (m/z): 500 (M + H)+. ¹H NMR (400 MHz, |
| | 4-(7-(5-methylpyridin-3-yl)-5- | CD₃OD) δ 8.88 (d, J = 2.3 Hz, 1H), 8.81 (d, J = 1.6 Hz, |
| | (pyridin-3-yl)-7H-pyrrolo[2,3- | 1H), 8.58 (dd, J = 4.9, 1.6 Hz, 1H), 8.47-8.44 (m, 2H), |
| | d]pyrimidin-4-yl)piperazine-1- | 8.18 (s, 1H), 8.12-8.08 (m, 1H), 7.91 (s, 1H), 7.59 (dd, J = |
| | carboxylate | 7.6, 5.3 Hz, 1H), 4.22-4.14 (m, 2H), 3.44-3.32 (m, 3H), |
| | | 3.05-2.88 (m, 1H), 2.50 (s, 3H), 1.43 (s, 9H), 1.12 (d, J = |
| | | 6.8 Hz, 3H), 0.98 (d, J = 6.7 Hz, 3H). |
| 179 | tert-butyl (2R,5S)-2,5-dimethyl- | LC/MS ESI (m/z): 554 (M + H)+. ¹H NMR (400 MHz, |
| | 4-(5-(pyridin-3-yl)-7-(5- | CD₃OD) δ 9.42 (d, J = 1.8 Hz, 1H), 9.15 (s, 1H), 8.98 (s, |
| | (trifluoromethyl)pyridin-3-yl)- | 1H), 8.88-8.80 (m, 3H), 8.59 (s, 1H), 8.32 (s, 1H), 8.23 |
| | 7H-pyrrolo[2,3-d]pyrimidin-4- | (dd, J = 8.1, 5.8 Hz, 1H), 4.56-4.43 (m, 1H), 4.16-4.09 |
| | yl)piperazine-1-carboxylate | (m, 1H), 3.63-3.51 (m, 1H), 3.29-3.23 (m, 3H), 1.43 (s, |
| | | 9H), 1.19 (d, J = 6.4 Hz, 3H), 0.97 (d, J = 5.9 Hz, 3H). |

### Example 64. Synthesis of tert-butyl (S)-4-(5-cyclopropyl-7-(3,5-difluorophenyl)-7H-pyrrolo[2,3-d]pyrimidin-4-yl)-2-(fluoromethyl)piperazine-1-carboxylate (Compound 269) and tert-butyl (R)-4-(5-cyclopropyl-7-(3,5-difluorophenyl)-7H-pyrrolo[2,3-d]pyrimidin-4-yl)-2-(fluoromethyl)piperazine-1-carboxylate (Compound 270)

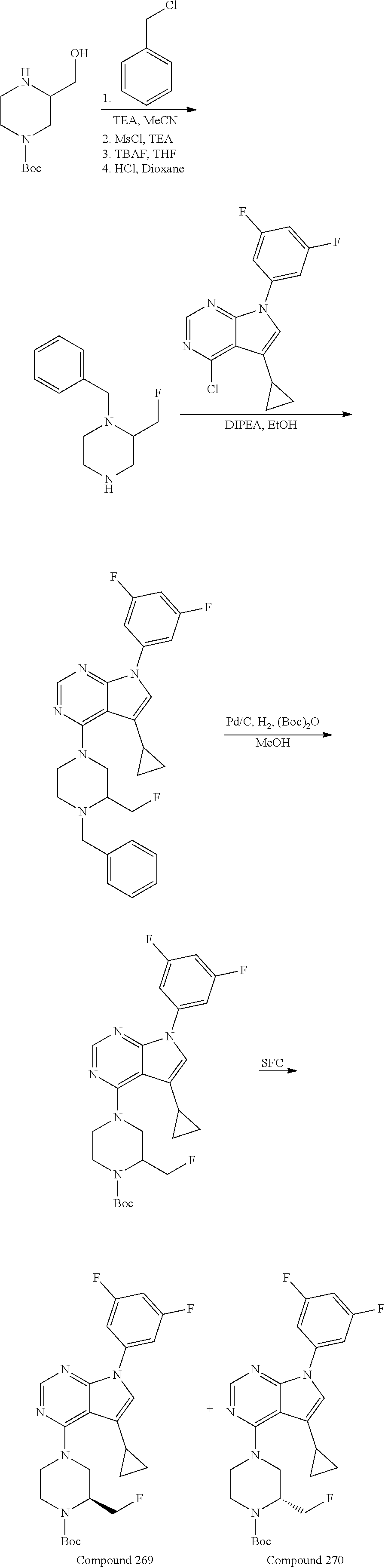

### Step 1. tert-Butyl 4-benzyl-3-(hydroxymethyl)piperazine-1-carboxylate

At 0° C., to a solution of tert-butyl 3-(hydroxymethyl)piperazine-1-carboxylate (4.5 g, 21 mmol) and TEA (4.2 mL, 42 mmol) in MeCN (50 mL) was added (chloromethyl)benzene (3.2 g, 25 mmol) dropwise. The reaction mixture was stirred at 80° C. overnight, LCMS showed the reaction was completed and the MeCN was removed by vacuum, the residue was purified by flash column chromatography (silica gel, 0˜70% EtOAc in petroleum ether) to afford tert-butyl 4-benzyl-3-(hydroxymethyl)piperazine-1-carboxylate (5.0 g, 71% yield) as a white solid. LCMS ESI (m/z): 307. (M+H)⁺.

### Step 2. tert-Butyl 4-benzyl-3-(((methylsulfonyl)oxy)methyl)piperazine-1-carboxylate

At 0° C., to a solution of tert-butyl 4-benzyl-3-(hydroxymethyl)piperazine-1-carboxylate (1.5 g, 5.0 mmol) in DCM (15 mL) was added TEA (0.75 mL, 7.5 mmol), followed by MsCl (0.68 g, 5.9 mmol) dropwise. The resulting mixture was stirred at room temperature for 2 h. The reaction was quenched with ice water, extracted with DCM twice. The combined organic layers were dried over Na₂SO₄, filtered and concentrated to give tert-butyl 4-benzyl-3-(((methylsulfonyl)oxy)methyl)piperazine-1-carboxylate (1.8 g, 94%) as an oil which was used in the next step without further purification.

### Step 3. tert-Butyl 4-benzyl-3-(fluoromethyl)piperazine-1-carboxylate

At 0° C., to a solution of tert-butyl 4-benzyl-3-(((methylsulfonyl)oxy)methyl)piperazine-1-carboxylate (1.8 g, 4.7 mmol) in THE (20 mL) was added TBAF (9.4 mL, 1.0M in THF) dropwise. The resulting mixture was stirred at room temperature overnight. The reaction was quenched with ice water, extracted with EtOAc twice. The combined organic layers were dried over Na₂SO₄, filtered and concentrated. The residue was purified by flash column chromatography (silica gel, 0-50%, ethyl acetate in petroleum ether) to give tert-butyl 4-benzyl-3-(fluoromethyl)piperazine-1-carboxylate (1.1 g, 76% yield) as an oil. LCMS ESI (m/z): 309 (M+H)⁺.

### Step 4. I-Benzyl-2-(fluoromethyl)piperazine

To a solution of tert-butyl 4-benzyl-3-(fluoromethyl)piperazine-1-carboxylate (300 mg, 0.97 mmol) in DCM (5 mL) was added HCl (4.0 mL, 4.0 M in dioxane). The resulting mixture was stirred at room temperature for 4 h. After removal of solvent, the residue was diluted with DCM, washed with NaHCO₃ (aq.), the organic layer was extracted with DCM twice, the combined organic layers were washed with brine, dried over Na₂SO₄, filtered and concentrated to give 1-benzyl-2-(fluoromethyl)piperazine which was used in the next step without further purification. LCMS ESI (m/z): 209 (M+H)⁺.

### Step 5. 4-(4-Benzylpiperazin-1-yl)-5-cyclopropyl-7-(3,5-difluorophenyl)-7H-pyrrolo[2,3-d]pyrimidine

A mixture of 4-chloro-5-cyclopropyl-7-(3,5-difluorophenyl)-7H-pyrrolo[2,3-d]pyrimidine (200 mg, 0.66 mmol, prepared following the procedure outlined in compound 256), 1-benzyl-2-(fluoromethyl)piperazine (200 mg, 0.98 mmol) and DIPEA (500 mg, 4.0 mmol) in EtOH (5 mL) was stirred at 100° C. overnight. After being cooled down to room temperature, solvent was removed. The residue was purified by flash column chromatography (silica gel, 0˜50% EtOAc in petroleum ether) to afford 4-(4-benzylpiperazin-1-yl)-5-cyclopropyl-7-(3,5-difluorophenyl)-7H-pyrrolo[2,3-d]pyrimidine (200 mg, 68% yield) as a white solid. LCMS ESI (m/z): 478 (M+H)⁺.

### Step 6. tert-Butyl 4-(5-cyclopropyl-7-(3,5-difluorophenyl)-7H-pyrrolo[2,3-d]pyrimidin-4-yl)-2-(fluoromethyl)piperazine-1-carboxylate

A mixture of 4-(4-benzylpiperazin-1-yl)-5-cyclopropyl-7-(3,5-difluorophenyl)-7H-pyrrolo[2,3-d]pyrimidine (200 mg, 0.45 mmol), (Boc)₂O (200 mg, 0.90 mmol) and Pd/C (50 mg, wetted with ca. 55% water) in MeOH (8 mL) was stirred at 40° C. overnight under H₂ atmosphere (˜1 atm). After being cooled down to room temperature, the reaction was filtered, filtrate was partitioned between EtOAc and water, organic layer was separated, the aqueous phase was extracted with EtOAc twice, dried over Na₂SO₄, filtered and concentrated, the residue was purified by flash chromatography (silica gel, 0˜50% EtOAc in petroleum ether) to give tert-butyl 4-(5-cyclopropyl-7-(3,5-difluorophenyl)-7H-pyrrolo[2,3-d]pyrimidin-4-yl)-2-(fluoromethyl)piperazine-1-carboxylate (96 mg, 44% yield) as a white solid. LCMS ESI (m/z): 488. (M+H)⁺.

### Step 7. tert-Butyl (S)-4-(5-cyclopropyl-7-(3,5-difluorophenyl)-7H-pyrrolo[2,3-d]pyrimidin-4-yl)-2-(fluoromethyl)piperazine-1-carboxylate and tert-butyl (r)-4-(5-cyclopropyl-7-(3,5-difluorophenyl)-7H-pyrrolo[2,3-d]pyrimidin-4-yl)-2-(fluoromethyl)piperazine-1-carboxylate

96 mg of tert-butyl 4-(5-cyclopropyl-7-(3,5-difluorophenyl)-7H-pyrrolo[2,3-d]pyrimidin-4-yl)-2-(fluoromethyl)piperazine-1-carboxylate was separated by SFC to give two isomers: Peak 1: shorter retention time, labeled as compound 269 (tert-butyl (S)-4-(5-cyclopropyl-7-(3,5-difluorophenyl)-7H-pyrrolo[2,3-d]pyrimidin-4-yl)-2-(fluoromethyl)piperazine-1-carboxylate (23 mg)): LC/MS ESI (m/z): 488. (M+H)⁺. ¹H NMR (400 MHz, CD₃OD) δ 8.35 (s, 1H), 7.57-7.52 (m, 2H), 7.33 (d, J=0.7 Hz, 1H), 6.97-6.91 (m, 1H), 4.63-4.47 (m, 4H), 4.30 (d, J=13.6 Hz, 1H), 4.02 (d, J=13.4 Hz, 1H), 3.45-3.35 (m, 2H), 3.17-3.10 (m, 1H), 2.08-2.02 (m, 1H), 1.49 (s, 9H), 1.07-1.03 (m, 2H), 0.99-0.93 (m, 1H), 0.72-0.67 (m, 1H).

Peak 2: longer retention time, labeled as compound 270 ((R)-4-(5-cyclopropyl-7-(3,5-difluorophenyl)-7H-pyrrolo[2,3-d]pyrimidin-4-yl)-2-(fluoromethyl)piperazine-1-carboxylate (22 mg)): LC/MS ESI (m/z): 488. (M+H)⁺, ¹H NMR (400 MHz, CD₃OD) δ 8.35 (s, 1H), 7.58-7.52 (m, 2H), 7.33 (s, 1H), 6.97-6.91 (m, 1H), 4.63-4.47 (m, 4H), 4.30 (d, J=13.6 Hz, 1H), 4.02 (d, J=13.4 Hz, 1H), 3.43-3.35 (m, 2H), 3.18-3.11 (m, 1H), 2.08-2.02 (m, 1H), 1.49 (s, 9H), 1.07-1.02 (m, 2H), 0.99-0.94 (m, 1H), 0.72-0.67 (m, 1H).

Preparative separation method: Instrument: Waters Thar 80 preparative SFC; Column: ChiralPak AD, 250×21.2 mm I.D., 5 μm; Mobile phase: A for CO₂ and B for MEOH+0.1% NH₃H₂O; Gradient: B 30%; Flow rate: 50 mL/min; Back pressure: 100 bar; Column temperature: 35° C.; Wavelength: 254 nm; Cycle-time: 3.0 min; Eluted time: 1.8 h.

### Example 65. Synthesis of tert-butyl (S)-4-(5-cyclopropyl-7-(3,5-difluorophenyl)-7H-pyrrolo[2,3-d]pyrimidin-4-yl)-2-(trifluoromethyl)piperazine-1-carboxylate (Compound 1006)

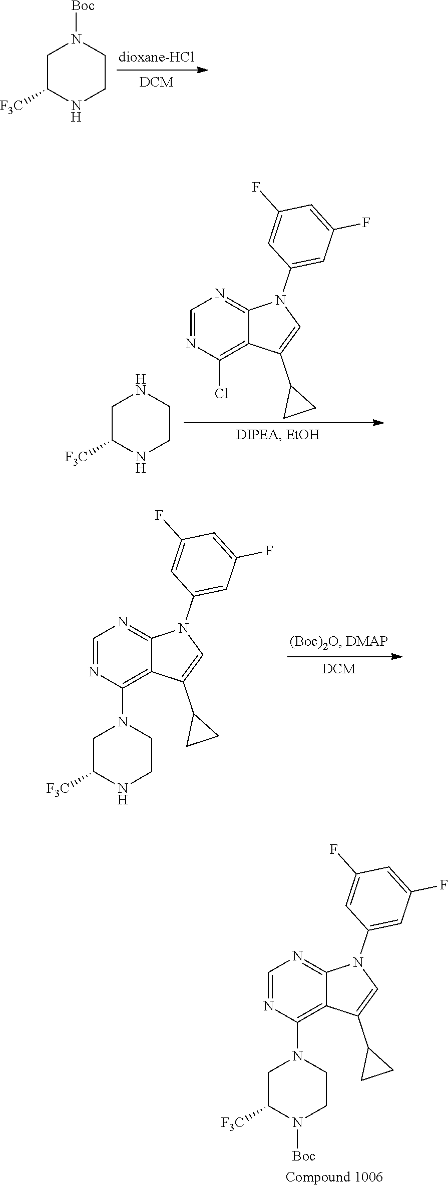 [SMILES: CC(C)(C)OC(=O)N1CCN[C@H](C(F)(F)F)C1 ; FC(F)(F)[C@@H]1CNCCN1 ; Fc1cc(F)cc(-n2cc(C3CC3)c3c(Cl)ncnc32)c1 ; Fc1cc(F)cc(-n2cc(C3CC3)c3c(N4CCN[C@H](C(F)(F)F)C4)ncnc32)c1 ; CC(C)(C)OC(=O)N1CCN(c2ncnc3c2c(C2CC2)cn3-c2cc(F)cc(F)c2)C[C@H]1C(F)(F)F]

### Step 1. (S)-2-(Trifluoromethyl)piperazine

To a solution of tert-butyl (S)-3-(trifluoromethyl)piperazine-1-carboxylate (60 mg, 0.27 mmol) in DCM (3 mL) was added HCl (2.0 mL, 4.0 M in dioxane). The resulting mixture was stirred at room temperature for 4 h. After removal of solvent, the residue was used in the next step directly. (S)-2-(trifluoromethyl)piperazine. LC/MS ESI (m/z): 155 (M+H)⁺.

### Step 2. (S)-5-Cyclopropyl-7-(3,5-difluorophenyl)-4-(3-(trifluoromethyl)piperazin-1-yl)-7H-pyrrolo[2,3-d]pyrimidine

To a solution of 4-chloro-5-cyclopropyl-7-(3,5-difluorophenyl)-7H-pyrrolo[2,3-d]pyrimidine (50 mg, 0.16 mmol, prepared following the procedure outlined in compound 256) in EtOH (5 mL) were added (S)-2-(trifluoromethyl)piperazine (38 mg, 0.24 mmol) and DIPEA (120 mg, 0.96 mmol). The resulting mixture was heated to 100° C. overnight. After being cooled down to room temperature, solvent was removed and the residue was purified by flash column chromatography (silica gel, 0˜50%, ethyl acetate in petroleum ether) to afford (S)-5-cyclopropyl-7-(3,5-difluorophenyl)-4-(3-(trifluoromethyl)piperazin-1-yl)-7H-pyrrolo[2,3-d]pyrimidine (60 mg, 86%) as a white solid. LC/MS ESI (m/z): 424 (M+H)⁺.

### Step 3. tert-Butyl (S)-4-(5-cyclopropyl-7-(3,5-difluorophenyl)-7H-pyrrolo[2,3-d]pyrimidin-4-yl)-2-(trifluoromethyl)piperazine-1-carboxylate

A mixture of (S)-5-cyclopropyl-7-(3,5-difluorophenyl)-4-(3-(trifluoromethyl)piperazin-1-yl)-7H-pyrrolo[2,3-d]pyrimidine (60 mg, 0.14 mmol), (Boc)₂O (61 mg, 0.28 mmol) DMAP (5.1 mg, 0.040 mmol) in THE (8 mL) was stirred at 40° C. overnight, under N₂. After being cooled down to room temperature, the reaction mixture was partitioned between EtOAc and water, organic layer was separated, the aqueous phase was extracted with EtOAc twice, dried over Na₂SO₄, filtered and concentrated, the residue was purified by flash chromatography (silica gel, 0-50% EtOAc in petroleum ether) to give tert-butyl (S)-4-(5-cyclopropyl-7-(3,5-difluorophenyl)-7H-pyrrolo[2,3-d]pyrimidin-4-yl)-2-(trifluoromethyl)piperazine-1-carboxylate (19 mg, 24% yield) as a white solid. LCMS ESI (m/z): 524 (M+H)⁺. ¹H NMR (400 MHz, CD₃OD) δ 8.37 (s, 1H), 7.58-7.52 (m, 2H), 7.34 (s, 1H), 6.99-6.87 (m, 1H), 4.95 (s, 1H), 4.58-4.45 (m, 2H), 4.14 (d, J=13.3 Hz, 1H), 3.60-3.44 (m, 2H), 3.19-3.12 (m, 1H), 2.06-1.99 (m, 1H), 1.48 (s, 9H), 1.06-0.98 (m, 3H), 0.70-0.63 (m, 1H)

The following compound was prepared by the procedure similar to the synthesis of compound 1006 from the corresponding amine.

| Cmpd | | |
|---|---|---|
| No. | Chemical Name | LCMS and ¹H NMR |
| 276 | tert-butyl (R)-4-(5-cyclopropyl- | LC/MS ESI (m/z): 524 (M + H)+. ¹H NMR (400 MHz, |
| | 7-(3,5-difluorophenyl)-7H- | CD₃OD) δ 8.37 (s, 1H), 7.59-7.52 (m, 2H), 7.34 (d, J = |
| | pyrrolo[2,3-d]pyrimidin-4-yl)-2- | 0.9 Hz, 1H), 6.98-6.91 (m, 1H), 4.94 (s, 1H), 4.57-4.43 |
| | (trifluoromethyl)piperazine-1- | (m, 2H), 4.14 (d, J = 13.2 Hz, 1H), 3.60-3.45 (m, 2H), |
| | carboxylate | 3.19-3.12 (m, 1H), 2.02 (d, J = 5.1 Hz, 1H), 1.48 (s, 9H), |
| | | 1.07-0.99 (m, 3H), 0.70-0.63 (m, 1H). |

### Example 66. Synthesis of tert-butyl (R)-4-(7-(3-cyanophenyl)-5-(pyrrolidin-1-yl)-7H-pyrrolo[2,3-d]pyrimidin-4-yl)-2-methylpiperazine-1-carboxylate (Compound 277)

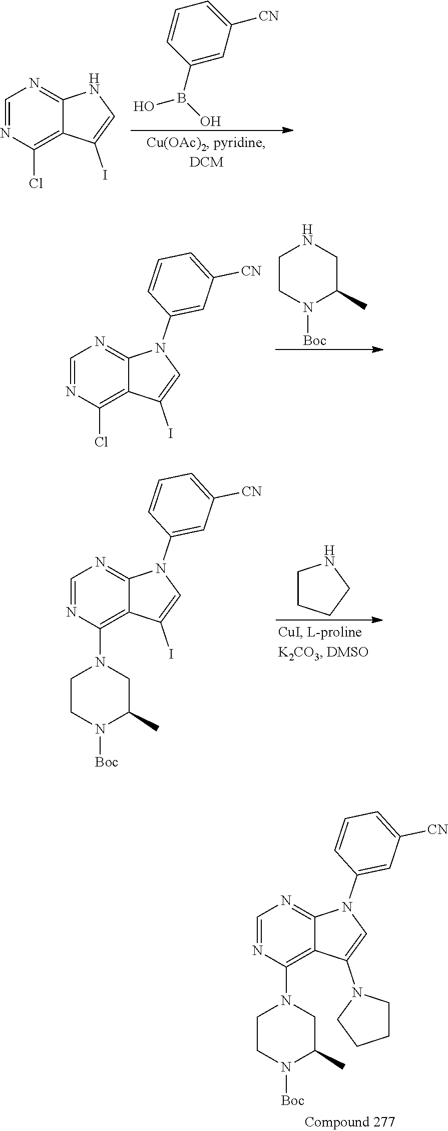

### Step 1. 3-(4-Chloro-5-iodo-7H-pyrrolo[2,3-d]pyrimidin-7-yl)benzonitrile

To a solution of 4-chloro-5-iodo-7H-pyrrolo[2,3-d]pyrimidine (4.7 g, 17 mmol) in DCM (500 mL) were added (3-cyanophenyl)boronic acid (5.0 g, 34 mmol), Cu(OAc)₂ (9.4 g, 51 mmol), pyridine (9.0 mL, 54 mmol). The resulting mixture was stirred at room temperature under O₂ atmosphere over weekend. The reaction was quenched with NH₄OH (25 mL) at ice water bath, filtered, the filtrate was extract with DCM twice, the combined organic layers were dried over Na₂SO₄, filtered and concentrated, the residue was purified by flash chromatography (silica gel, 0˜50%, ethyl acetate in petroleum ether) to afford 3-(4-chloro-5-iodo-7H-pyrrolo[2,3-d]pyrimidin-7-yl)benzonitrile (5.0 g, 78%) as a white solid. LC/MS ESI (m/z): 381 (M+H)⁺.

### Step 2. tert-Butyl (R)-4-(7-(3-cyanophenyl)-5-iodo-7H-pyrrolo[2,3-d]pyrimidin-4-yl)-2-methylpiperazine-1-carboxylate

To a solution of 3-(4-chloro-5-iodo-7H-pyrrolo[2,3-d]pyrimidin-7-yl)benzonitrile (100 mg, 0.30 mmol) in EtOH (5 mL) were added DIEA (230 mg, 1.8 mmol) and tert-butyl (R)-2-methylpiperazine-1-carboxylate (240 mg, 1.2 mmol). The resulting mixture was heated to 100° C. overnight. After being cooled down to room temperature, solvent was removed and the residue was purified by flash column chromatography (silica gel, 0˜20%, ethyl acetate in petroleum ether) to afford tert-butyl (R)-4-(7-(3-cyanophenyl)-5-iodo-7H-pyrrolo[2,3-d]pyrimidin-4-yl)-2-methylpiperazine-1-carboxylate (100 mg, 70%) as a white solid. LC/MS ESI (m/z): 545 (M+H)⁺.

### Step 3. tert-Butyl (R)-4-(7-(3-cyanophenyl)-5-(pyrrolidin-1-yl)-7H-pyrrolo[2,3-d]pyrimidin-4-yl)-2-methylpiperazine-1-carboxylate

To a solution of tert-butyl (R)-4-(7-(3-cyanophenyl)-5-iodo-7H-pyrrolo[2,3-d]pyrimidin-4-yl)-2-methylpiperazine-1-carboxylate (30 mg, 0.10 mmol) in DMSO (5 mL) were added CuI (2.0 mg, 0.010 mmol), K₂CO₃ (23 mg, 0.17 mmol), L-proline (3.0 mg, 0.020 mmol) and pyrrolidine (71 mg, 1.0 mmol). The resulting mixture was heated to 90° C. overnight. After being cooled down to room temperature, the reaction was diluted with water, extracted with EtOAc twice, the combined organic layers were washed with brine, dried over Na₂SO₄, filtered and concentrated, the residue was purified by flash chromatography (silica gel, 0˜20%, ethyl acetate in petroleum ether) to give crude product which was further purified by prep-HPLC to afford tert-butyl (R)-4-(7-(3-cyanophenyl)-5-(pyrrolidin-1-yl)-7H-pyrrolo[2,3-d]pyrimidin-4-yl)-2-methylpiperazine-1-carboxylate (8.1 mg, 31%) as a white solid. LC/MS ESI (m/z): 488 (M+H)⁺. ¹H NMR (400 MHz, CD₃OD) δ 8.26 (s, 2H), 8.13-8.07 (m, 1H), 7.71-7.66 (m, 2H), 7.11 (s, 1H), 4.82-4.78 (m, 1H), 4.48 (d, J=13.5 Hz, 1H), 4.37-4.31 (m, 1H), 3.89 (d, J=13.7 Hz, 1H), 3.39-3.32 (m, 2H), 3.29-3.25 (m, 2H), 3.02 (dt, J=16.1, 8.0 Hz, 1H), 2.91-2.84 (m, 2H), 2.04-1.98 (m, 4H), 1.48 (s, 9H), 1.08 (d, J=6.8 Hz, 3H).

The following compound was prepared by the procedure similar to the synthesis of compound 277 from corresponding amines.

| Cmpd | | |
|---|---|---|
| No. | Chemical Name | LCMS and ¹H NMR |
| 285 | tert-butyl (S)-4-(7-(3- | LC/MS ESI (m/z): 488 (M + H)+. ¹H NMR (400 MHz, |
| | cyanophenyl)-5-(pyrrolidin-1- | CD₃OD) δ 8.30-8.22 (m, 2H), 8.13-8.08 (m, 1H), 7.71- |
| | yl)-7H-pyrrolo[2,3-d]pyrimidin- | 7.66 (m, 2H), 7.12 (s, 1H), 5.13-5.03 (m, 1H), 4.24-4.09 |
| | 4-yl)-3-methylpiperazine-1- | (m, 2H), 3.96-3.91 (m, 1H), 3.49-3.35 (m, 2H), 3.26- |
| | carboxylate | 3.20 (m, 2H), 3.16-3.05 (m, 1H), 2.98-2.91 (m, 2H), |
| | | 2.05-1.99 (m, 4H), 1.49 (s, 9H), 1.10 (d, J = 6.5 Hz, 3H). |
| 307 | tert-butyl (S)-4-(7-(3,5- | LC/MS ESI (m/z): 529 (M + H)+. ¹H NMR (400 MHz, |
| | difluorophenyl)-5-((R)-2- | CD₃OD) δ 8.29 (s, 1H), 7.62-7.56 (m, 2H), 7.23 (s, 1H), |
| | (hydroxymethyl)pyrrolidin-1- | 6.91 (tt, J = 9.1, 2.3 Hz, 1H), 5.28-5.17 (m, 1H), 4.16- |
| | yl)-7H-pyrrolo[2,3-d]pyrimidin- | 4.06 (m, 1H), 3.99-3.87 (m, 2H), 3.82-3.74 (m, 1H), |
| | 4-yl)-3-methylpiperazine-1- | 3.67-3.61 (m, 1H), 3.58-3.39 (m, 4H), 3.10-2.88 (m, |
| | carboxylate | 1H), 2.77-2.67 (m, 1H), 2.22-2.14 (m, 1H), 2.00-1.88 |
| | | (m, 3H), 1.49 (s, 9H), 1.01 (d, J = 6.1 Hz, 3H). |
| 324 | tert-butyl (S)-4-(7-(3- | LC/MS ESI (m/z): 504 (M + H)+. ¹H NMR (400 MHz, |
| | cyanophenyl)-5-morpholino-7H- | CD₃OD) δ 8.29 (s, 1H), 8.26-8.24 (m, 1H), 8.13-8.09 |
| | pyrrolo[2,3-d]pyrimidin-4-yl)-3- | (m, 1H), 7.70 (d, J = 5.0 Hz, 2H), 7.22 (s, 1H), 5.20-5.12 |
| | methylpiperazine-1-carboxylate | (m, 1H), 4.33-4.22 (m, 1H), 4.13-4.07 (m, 1H), 3.96- |
| | | 3.87 (m, 5H), 3.51-3.41 (m, 2H), 3.20-3.12 (m, 2H), |
| | | 3.06-2.86 (m, 3H), 1.50 (s, 9H), 1.12 (d, J = 6.4 Hz, 3H). |
| 244 | tert-butyl (S)-4-(7-(3- | LC/MS ESI (m/z): 517 (M + H)+. ¹H NMR (400 MHz, |
| | cyanophenyl)-5-(4- | CD₃OD) δ 8.31 (s, 1H), 8.25-8.23 (m, 1H), 8.13-8.08 |
| | methylpiperazin-1-yl)-7H- | (m, 1H), 7.72-7.69 (m, 2H), 7.27 (s, 1H), 5.15-5.01 (m, |
| | pyrrolo[2,3-d]pyrimidin-4-yl)-3- | 1H), 4.29-4.15 (m, 1H), 4.12-4.06 (m, 1H), 3.96-3.91 |
| | methylpiperazine-1-carboxylate | (m, 1H), 3.56-3.33 (m, 4H), 3.17-2.86 (m, 7H), 2.64 (s, |
| | | 3H), 1.50 (s, 9H), 1.15 (d, J = 5.7 Hz, 3H). |
| 363 | tert-butyl (S)-4-(7-(3- | LC/MS ESI (m/z): 518 (M + H)+. ¹H NMR (400 MHz, |
| | cyanophenyl)-5-((R)-2- | CD₃OD) δ 8.29 (s, 1H), 8.26-8.23 (m, 1H), 8.13-8.08 |
| | methylmorpholino)-7H- | (m, 1H), 7.69 (d, J = 5.1 Hz, 2H), 7.20 (s, 1H), 5.19-5.08 |
| | pyrrolo[2,3-d]pyrimidin-4-yl)-3- | (m, 1H), 4.33-4.20 (m, 1H), 4.09 (d, J = 12.6 Hz, 1H), |
| | methylpiperazine-1-carboxylate | 4.01 (dd, J = 11.5, 2.0 Hz, 1H), 3.96-3.86 (m, 3H), 3.54- |
| | | 3.43 (m, 2H), 3.41-3.32 (m, 1H), 3.18 (d, J = 11.2 Hz, |
| | | 1H), 3.07-2.86 (m, 1H), 2.78 (td, J = 11.5, 3.3 Hz, 1H), |
| | | 2.21 (t, J = 1.2 Hz, 1H), 1.50 (s, 9H), 1.19 (d, J = 6.3 Hz, |
| | | 3H), 1.14 (d, J = 6.5 Hz, 3H). |
| 369 | tert-butyl (S)-4-(7-(3- | LC/MS ESI (m/z): 518 (M + H)+. ¹H NMR (400 MHz, |
| | cyanophenyl)-5-((R)-3- | CD₃OD) δ 8.28 (s, 1H), 8.22-8.20 (m, 1H), 8.08-8.05 |
| | methylmorpholino)-7H- | (m, 1H), 7.77-7.70 (m, 2H), 7.64 (s, 1H), 5.52-5.08 (m, |
| | pyrrolo[2,3-d]pyrimidin-4-yl)-3- | 1H), 4.82-4.65 (m, 1H), 4.23-4.14 (m, 1H), 4.01-3.96 |
| | methylpiperazine-1-carboxylate | (m, 1H), 3.93-3.88 (m, 2H), 3.82-3.74 (m, 1H), 3.49- |
| | | 3.41 (m, 2H), 3.28-3.06 (m, 4H), 3.05-2.98 (m, 1H), 1.51 |
| | | (s, 9H), 1.27 (d, J = 6.6 Hz, 3H), 0.96 (d, J = 6.3 Hz, 3H). |
| 379 | tert-butyl (S)-4-(7-(3- | LC/MS ESI (m/z): 518 (M + H)+. ¹H NMR (400 MHz, |
| | cyanophenyl)-5-((S)-3- | CD₃OD) δ 8.30 (s, 1H), 8.27-8.22 (m, 1H), 8.12-8.07 |
| | methylmorpholino)-7H- | (m, 1H), 7.75-7.70 (m, 2H), 7.51-7.37 (m, 1H), 5.51- |
| | pyrrolo[2,3-d]pyrimidin-4-yl)-3- | 5.31 (m, 1H), 4.32-4.19 (m, 1H), 4.16-4.10 (m, 1H), |
| | methylpiperazine-1-carboxylate | 3.98-3.93 (m, 2H), 3.89-3.84 (m, 2H), 3.54-3.41 (m, |
| | | 3H), 3.28-3.21 (m, 2H), 3.09-2.95 (m, 1H), 2.84-2.77 |
| | | (m, 1H), 1.50 (s, 9H), 1.12 (d, J = 6.3 Hz, 3H), 1.01 (d, J = |
| | | 6.3 Hz, 3H). |

### Example 67. Synthesis of tert-butyl (S)-4-(1-(3-chlorophenyl)-3-(tetrahydro-2H-pyran-4-yl)-1H-pyrazolo[3,4-d]pyrimidin-4-yl)-3-methylpiperazine-1-carboxylate (Compound 284)

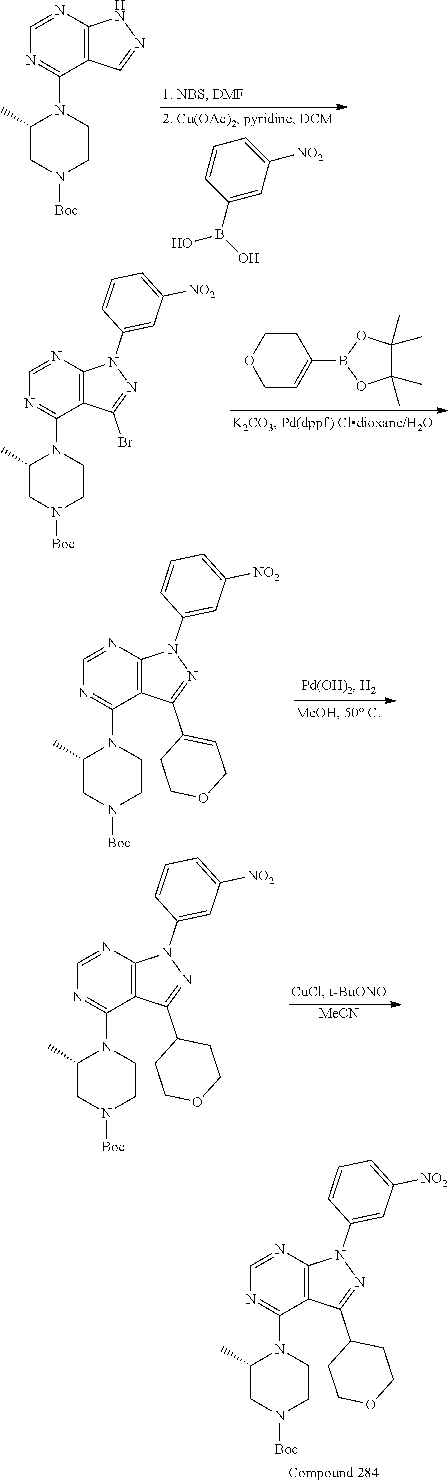

### Step 1. tert-Butyl (S)-4-(3-bromo-1H-pyrazolo[3,4-d]pyrimidin-4-yl)-3-methylpiperazine-1-carboxylate

To a suspension of tert-butyl (S)-3-methyl-4-(1H-pyrazolo[3,4-d]pyrimidin-4-yl)piperazine-1-carboxylate (1.3 g, 4.1 mmol) in anhydrous DMF (13 mL) was added NBS (0.87 g, 4.9 mmol). The resulting mixture was stirred at 70° C. for 2 hr. The reaction was diluted with water, extracted with EtOAc twice. The combined organic layers were washed with brine, dried over Na₂SO₄, filtered and concentrated. The residue was purified by flash column chromatography (silica gel, 0-40%, ethyl acetate in petroleum ether) to afford tert-butyl (S)-4-(3-bromo-1H-pyrazolo[3,4-d]pyrimidin-4-yl)-3-methylpiperazine-1-carboxylate (1.0 g, 61%) as a light yellow oil. LC/MS ESI (m/z): 397,399 (M+H)⁺.

### Step 2. tert-Butyl (S)-4-(3-bromo-1-(3-nitrophenyl)-1H-pyrazolo[3,4-d]pyrimidin-4-yl)-3-methylpiperazine-1-carboxylate

To a solution of tert-butyl (S)-4-(3-bromo-1H-pyrazolo[3,4-d]pyrimidin-4-yl)-3-methylpiperazine-1-carboxylate (500 mg, 1.3 mmol) in DCM (20 mL) were added (3-nitrophenyl)boronic acid (400 mg, 2.4 mmol), Cu(OAc)₂ (540 mg, 3.1 mmol), pyridine (0.60 mL, 7.2 mmol) and 4 A molecular sieves (500 mg). The resulting mixture was heated to 40° C. over the weekend under O₂ atmosphere. After being cooled down to room temperature, the reaction was quenched with NH₄OH, diluted with DCM, and then filtered. The filtrate was extracted with DCM twice, the combined organic layers were washed with brine, dried over Na₂SO₄, filtered and concentrated. The residue was purified by flash column chromatography (silica gel, 0˜80%, ethyl acetate in petroleum ether) to afford tert-butyl (S)-4-(3-bromo-1-(3-nitrophenyl)-1H-pyrazolo[3,4-d]pyrimidin-4-yl)-3-methylpiperazine-1-carboxylate (430 mg, 66%) as a light yellow solid. LC/MS ESI (m/z): 518,520 (M+H)⁺

### Step 3. tert-Butyl (S)-4-(3-(3,6-dihydro-2H-pyran-4-yl)-1-(3-nitrophenyl)-1H-pyrazolo[3,4-d]pyrimidin-4-yl)-3-methylpiperazine-1-carboxylate

To a solution of tert-butyl (S)-4-(3-bromo-1-(3-nitrophenyl)-1H-pyrazolo[3,4-d]pyrimidin-4-yl)-3-methylpiperazine-1-carboxylate (430 mg, 0.83 mmol) in dioxane (15 mL) and H₂O (3 mL) were added 2-(3,6-dihydro-2H-pyran-4-yl)-4,4,5,5-tetramethyl-1,3,2-dioxaborolane (210 mg, 0.99 mmol), K₂CO₃ (340 mg, 2.5 mmol) and Pd(dppf)Cl₂ (58 mg, 0.080 mmol). The resulting mixture was stirred at 90° C. overnight under N₂. After being cooled down to room temperature, solvent was removed and the residue was purified by flash column chromatography (silica gel, 0-40%, ethyl acetate in petroleum ether) to afford tert-butyl (S)-4-(3-(3,6-dihydro-2H-pyran-4-yl)-1-(3-nitrophenyl)-1H-pyrazolo[3,4-d]pyrimidin-4-yl)-3-methylpiperazine-1-carboxylate (410 mg, 96%) as a yellow solid. LC/MS ESI (m/z): 522 (M+H)⁺.

### Step 4. tert-Butyl (S)-4-(1-(3-aminophenyl)-3-(tetrahydro-2H-pyran-4-yl)-1H-pyrazolo[3,4-d]pyrimidin-4-yl)-3-methylpiperazine-1-carboxylate

To a solution of tert-butyl (S)-4-(3-(3,6-dihydro-2H-pyran-4-yl)-1-(3-nitrophenyl)-1H-pyrazolo[3,4-d]pyrimidin-4-yl)-3-methylpiperazine-1-carboxylate (410 mg, 0.78 mmol) in MeOH (5 mL) was added Pd(OH)₂ (200 mg, 1.4 mmol). The resulting mixture was heated to 50° C. overnight under H₂ atmosphere. After being cooled down to room temperature, the reaction mixture was filtered and concentrated. The residue was used in the next step directly. tert-butyl (S)-4-(1-(3-aminophenyl)-3-(tetrahydro-2H-pyran-4-yl)-1H-pyrazolo[3,4-d]pyrimidin-4-yl)-3-methylpiperazine-1-carboxylate (350 mg, 91%) as a light yellow solid. LC/MS ESI (m/z): 494 (M+H)⁺.

### Step 5. tert-Butyl (S)-4-(1-(3-chlorophenyl)-3-(tetrahydro-2H-pyran-4-yl)-1H-pyrazolo[3,4-d]pyrimidin-4-yl)-3-methylpiperazine-1-carboxylate

At 0° C., to a solution of tert-butyl (S)-4-(1-(3-aminophenyl)-3-(tetrahydro-2H-pyran-4-yl)-1H-pyrazolo[3,4-d]pyrimidin-4-yl)-3-methylpiperazine-1-carboxylate (330 mg, 0.66 mmol) in MeCN (5 mL) was added CuCl (76 mg, 0.79 mmol), after stirred at 0° C. for 5 min, t-BuONO (0.1 mL, 0.9 mmol) was added dropwise. The resulting mixture was heated to 60° C. for 1 hr. After being cooled down to room temperature, the reaction was partitioned between EtOAc and water, organic layer was separated, aqueous layer was extracted with EtOAc twice, the combined organic layers were washed with brine, dried over Na₂SO₄, filtered and concentrated, the residue was purified by flash column chromatography (silica gel, ethyl acetate in petroleum ether) to give crude product which was further purified by prep-HPLC to afford tert-butyl (S)-4-(1-(3-chlorophenyl)-3-(tetrahydro-2H-pyran-4-yl)-1H-pyrazolo[3,4-d]pyrimidin-4-yl)-3-methylpiperazine-1-carboxylate (37.2 mg, 11%) as a white solid. LC/MS ESI (m/z): 513 (M+H)⁺. H NMR (400 MHz, CD₃OD) δ 8.46 (s, 1H), 8.31 (t, J=2.0 Hz, 1H), 8.18 (ddd, J=8.3, 2.0, 0.9 Hz, 1H), 7.49 (t, J=8.1 Hz, 1H), 7.32 (ddd, J=8.0, 2.0, 0.9 Hz, 1H), 4.59 (qd, J=6.3, 2.8 Hz, 1H), 4.14-4.04 (m, 3H), 3.92 (t, J=13.7 Hz, 2H), 3.69-3.57 (m, 3H), 3.38-3.32 (m, 2H), 3.19-3.05 (m, 1H), 2.13-2.06 (m, 3H), 1.94-1.84 (m, 1H), 1.50 (s, 9H), 1.32 (d, J=6.7 Hz, 3H).

### Example 68. Synthesis of tert-butyl (R)-2-methyl-4-(5-(pyrrolidin-1-yl)-7-(3-(trifluoromethoxy)phenyl)-7H-pyrrolo[2,3-d]pyrimidin-4-yl)piperazine-1-carboxylate (Compound 311)

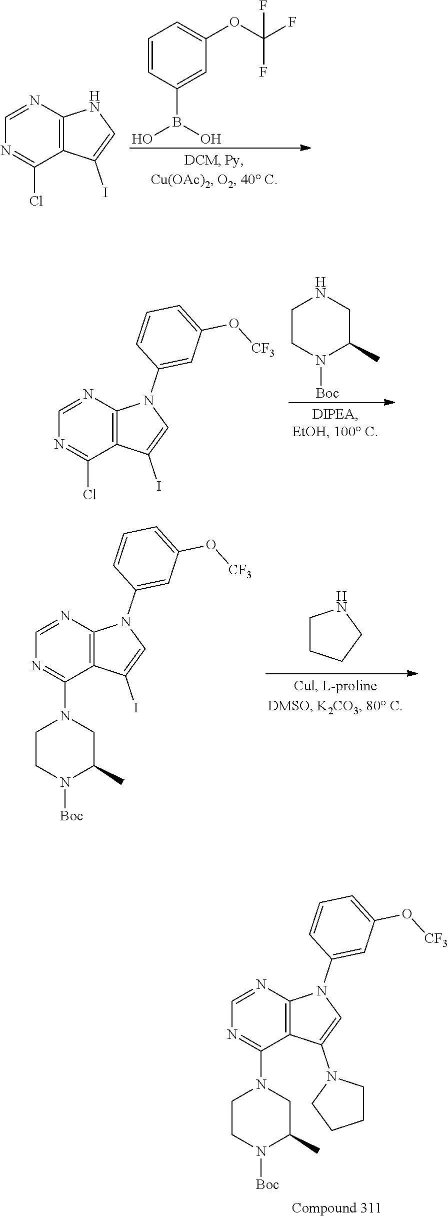

### Step 1. 4-Chloro-5-iodo-7-(3-(trifluoromethoxy)phenyl)-7H-pyrrolo[2,3-d]pyrimidine

To a solution of 4-chloro-5-iodo-7H-pyrrolo[2,3-d]pyrimidine (900 mg, 3.2 mmol) in DCM (25 mL) were added (3-(trifluoromethoxy)phenyl)boronic acid (1.3 g, 6.4 mmol), 4 A molecular sieves (800 mg), Cu(OAc)₂ (2.6 g, 12.8 mmol) and pyridine (1.5 g, 19.2 mmol). The resulting mixture was stirred at 40° C. under O₂ atmosphere for 2 days. The reaction was quenched with NH₄OH (3 mL) at ice water bath and then was filtered, the filtrate was extract with DCM twice, the combined organic layers were dried over Na₂SO₄, filtered and concentrated, the residue was purified by flash column chromatography (silica gel, 0˜30% EtOAc in petroleum ether) to afford 4-chloro-5-iodo-7-(3-(trifluoromethoxy)phenyl)-7H-pyrrolo[2,3-d]pyrimidine (540 mg, 38%) as a yellow solid. LC/MS ESI (m/z): 440 (M+H)⁺.

### Step 2. tert-Butyl (R)-4-(5-iodo-7-(3-(trifluoromethoxy)phenyl)-7H-pyrrolo[2,3-d]pyrimidin-4-yl)-2-methylpiperazine-1-carboxylate

To the solution of 4-chloro-5-iodo-7-(3-(trifluoromethoxy)phenyl)-7H-pyrrolo[2,3-d]pyrimidine (260 mg, 0.59 mmol) in EtOH (5 mL) were added tert-butyl (R)-2-methylpiperazine-1-carboxylate (236 mg, 1.18 mmol), DIPEA (229 mg, 1.77 mmol). The resulting reaction mixture was stirred at 100° C. under N₂ for overnight. After removal of solvent, the crude product was purified by flash column chromatography (silica gel, 0˜30%, ethyl acetate in petroleum ether) to afford tert-butyl (R)-4-(5-iodo-7-(3-(trifluoromethoxy)phenyl)-7H-pyrrolo[2,3-d]pyrimidin-4-yl)-2-methylpiperazine-1-carboxylate (220 mg, 61%) as a solid. LC/MS ESI (m/z): 604 (M+H)⁺.

### Step 3. tert-Butyl (R)-2-methyl-4-(5-(pyrrolidin-1-yl)-7-(3-(trifluoromethoxy)phenyl)-7H-pyrrolo[2,3-d]pyrimidin-4-yl)piperazine-1-carboxylate

To the solution of tert-butyl (R)-4-(5-iodo-7-(3-(trifluoromethoxy)phenyl)-7H-pyrrolo[2,3-d]pyrimidin-4-yl)-2-methylpiperazine-1-carboxylate (220 mg, 0.36 mmol) in DMSO (3 mL) were added CuI (14 mg, 0.07 mmol), L-proline (17 mg, 0.14 mmol), K₂CO₃ (115 mg, 1.08 mmol) and pyrrolidine (103 mg, 1.44 mmol). The resulting reaction mixture was stirred at 80° C. under N₂ overnight. After which the reaction was extracted with EtOAc and water. The combined organic layers were dried over Na₂SO₄, filtered, and concentrated under reduced pressure. The crude product was purified by flash column chromatography (silica gel, 0˜30%, EtOAc in petroleum ether) to afford tert-butyl (R)-2-methyl-4-(5-(pyrrolidin-1-yl)-7-(3-(trifluoromethoxy)phenyl)-7H-pyrrolo[2,3-d]pyrimidin-4-yl)piperazine-1-carboxylate (23 mg, 12%) as a solid. LC/MS ESI (m/z): 547 (M+H)⁺. ¹H NMR (400 MHz, CD₃OD) δ 8.24 (s, 1H), 7.82 (s, 1H), 7.74 (d, J=8.9 Hz, 1H), 7.59 (t, J=8.2 Hz, 1H), 7.25 (d, J=8.2 Hz, 1H), 7.06 (s, 1H), 4.79 (d, 1H), 4.47 (d, J=13.3 Hz, 1H), 4.37-4.32 (m, 1H), 3.89 (d, J=13.4 Hz, 1H), 3.39-3.32 (m, 1H), 3.29-3.24 (m, 3H), 3.04-2.96 (m, 1H), 2.90-2.84 (m, 2H), 2.03-1.97 (m, 4H), 1.48 (s, 9H), 1.08 (d, J=6.8 Hz, 3H).

The following compounds were prepared by the procedures analogous to the synthesis of compound 311 from the corresponding amines.

| Cmpd | | |
|---|---|---|
| No. | Chemical Name | LCMS and ¹H NMR |
| 300 | tert-butyl (R)-4-(7-(3- | LC/MS ESI (m/z): 533 (M + H)⁺. ¹H NMR (400 MHz, |
| | chlorophenyl)-5-(3,3- | CDCl₃) δ 8.39 (s, 1H), 7.72 (t, J = 2.0 Hz, 1H), 7.67-7.62 |
| | difluoropyrrolidin-1-yl)-7H- | (m, 1H), 7.43 (t, J = 8.1 Hz, 1H), 7.32-7.29 (m, 1H), 6.75 |
| | pyrrolo[2,3-d]pyrimidin-4-yl)-2- | (s, 1H), 4.71-4.65 (m, 1H), 4.43-4.35 (m, 1H), 4.35-4.30 |
| | methylpiperazine-1-carboxylate | (m, 1H), 3.93 (d, J = 13.2 Hz, 1H), 3.69-3.60 (m, |
| | | 1H), 3.49-3.43 (m, 1H), 3.42-3.35 (m, 1H), 3.34-3.30 |
| | | (m, 1H), 3.29-3.20 (m, 1H), 3.17-3.10 (m, 1H), 3.05-2.97 |
| | | (m, 1H), 2.56-2.45 (m, 2H), 1.49 (s, 9H), 1.07 (d, J = |
| | | 6.8 Hz, 3H). |
| 341 | tert-butyl (R)-4-(5-(azetidin-1- | LC/MS ESI (m/z): 474 (M + H)⁺. ¹H NMR (400 MHz, |
| | yl)-7-(3-cyanophenyl)-7H- | CD₃OD) δ 8.26-8.24 (m, 1H), 8.23 (s, 1H), 8.11-8.07 |
| | pyrrolo[2,3-d]pyrimidin-4-yl)-2- | (m, 1H), 7.70-7.65 (m, 2H), 7.02 (s, 1H), 4.81-4.75 (m, |
| | methylpiperazine-1-carboxylate | 1H), 4.58-4.52 (m, 1H), 4.35-4.29 (m, 1H), 3.94-3.84 |
| | | (m, 3H), 3.62 (q, J = 6.9 Hz, 2H), 3.46-3.38 (m, 2H), 3.06 |
| | | (td, J = 12.6, 3.7 Hz, 1H), 2.34-2.27 (m, 2H), 1.48 (s, 9H), |
| | | 1.01 (d, J = 6.8 Hz, 3H). |
| 306 | tert-butyl (S)-4-(7-(5-chloro-2- | LC/MS ESI (m/z): 511 (M + H)⁺. ¹H NMR (400 MHz, |
| | methylphenyl)-5-(pyrrolidin-1- | CD₃OD) δ 8.15-8.11 (m, 1H), 7.42-7.37 (m, 2H), 7.33 |
| | yl)-7H-pyrrolo[2,3-d]pyrimidin- | (d, J = 1.7 Hz, 1H), 6.79 (s, 1H), 5.13 (s, 1H), 4.27 (s, 1H), |
| | 4-yl)-3-methylpiperazine-1- | 4.13 (d, J = 12.7 Hz, 1H), 3.96-3.91 (m, 1H), 3.45-3.32 |
| | carboxylate | (m, 2H), 3.19-3.00 (m, 3H), 2.96-2.89 (m, 2H), 2.01 (s, |
| | | 3H), 2.01-1.97 (m, 4H), 1.50 (s, 9H), 1.12 (d, J = 6.5 Hz, |
| | | 3H). |
| 308 | tert-butyl (S)-4-(7-(3- | LC/MS ESI (m/z): 533 (M + H)⁺. ¹H NMR (400 MHz, |
| | chlorophenyl)-5-(3,3- | CD₃OD) δ 8.28 (s, 1H), 7.87 (t, J = 2.0 Hz, 1H), 7.70-7.66 |
| | difluoropyrrolidin-1-yl)-7H- | (m, 1H), 7.50 (t, J = 8.1 Hz, 1H), 7.39-7.34 (m, 1H), 7.20 |
| | pyrrolo[2,3-d]pyrimidin-4-yl)-3- | (s, 1H), 5.00 (s, 1H), 4.15-4.06 (m, 2H), 3.91 (d, J = 13.3 |
| | methylpiperazine-1-carboxylate | Hz, 1H), 3.66 (q, J = 12.0 Hz, 1H), 3.53-3.32 (m, 4H), |
| | | 3.24-3.18 (m, 1H), 3.13-2.99 (m, 1H), 2.56-2.45 (m, |
| | | 2H), 1.49 (s, 9H), 1.11 (d, J = 6.6 Hz, 3H). |
| 331 | tert-butyl (S)-3-methyl-4-(5- | LC/MS ESI (m/z): 547 (M + H)⁺. ¹H NMR (400 MHz, |
| | (pyrrolidin-1-yl)-7-(3- | CD₃OD) δ 8.25 (s, 1H), 7.83 (s, 1H), 7.74 (dd, J = 8.2, 1.3 |
| | (trifluoromethoxy)phenyl)-7H- | Hz, 1H), 7.59 (t, J = 8.2 Hz, 1H), 7.27-7.23 (m, 1H), 7.06 |
| | pyrrolo[2,3-d]pyrimidin-4- | (s, 1H), 5.08 (s, 1H), 4.22-4.09 (m, 2H), 3.95-3.90 (m, |
| | yl)piperazine-1-carboxylate | 1H), 3.44-3.32 (m, 2H), 3.25-3.19 (m, 2H), 3.05 (s, 1H), |
| | | 2.96-2.89 (m, 2H), 2.02-1.98 (m, 4H), 1.49 (s, 9H), 1.09 |
| | | (d, J = 6.5 Hz, 3H). |
| 350 | tert-butyl (S)-4-(5-(azetidin-1- | LC/MS ESI (m/z): 474 (M + H)⁺. ¹H NMR (400 MHz, |
| | yl)-7-(3-cyanophenyl)-7H- | CD₃OD) δ 8.27 (s, 2H), 8.13-8.09 (m, 1H), 7.71-7.65 |
| | pyrrolo[2,3-d]pyrimidin-4-yl)-3- | (m, 2H), 7.05 (s, 1H), 5.05 (s, 1H), 4.18-4.06 (m, 2H), |
| | methylpiperazine-1-carboxylate | 3.95-3.84 (m, 3H), 3.65 (q, J = 6.8 Hz, 2H), 3.53-3.43 |
| | | (m, 2H), 3.11-2.98 (m, 1H), 2.34-2.27 (m, 2H), 1.50 (s, |
| | | 9H), 1.12 (d, J = 6.6 Hz, 3H). |

### Example 69. Synthesis of tert-butyl (R)-2-methyl-4-(5-(2-oxopyrrolidin-1-yl)-7-(3,4,5-trifluorophenyl)-7H-pyrrolo[2,3-d]pyrimidin-4-yl)piperazine-1-carboxylate (Compound 313)

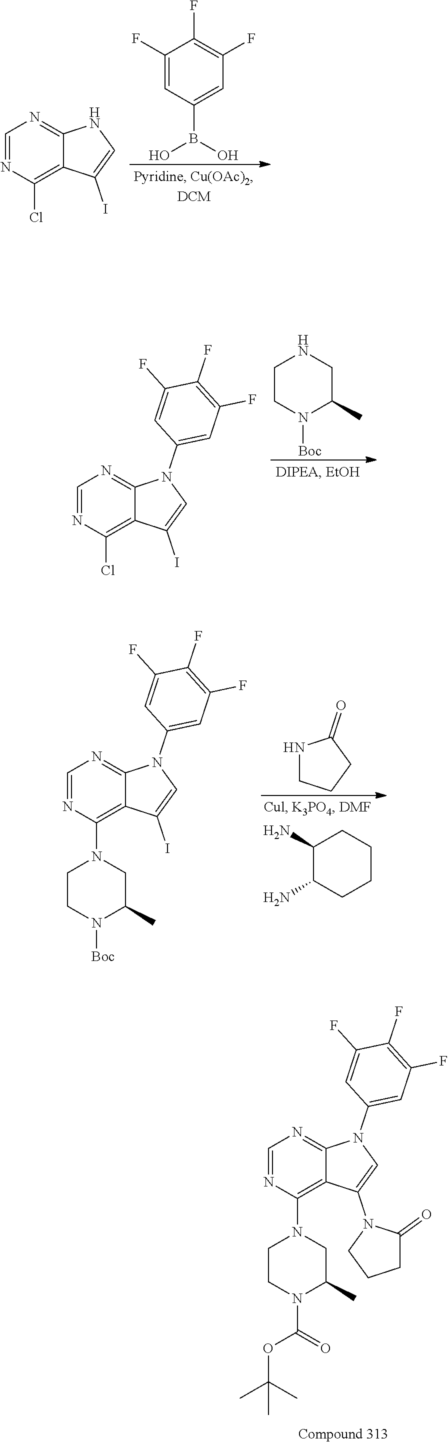

### Step 1. 4-Chloro-5-iodo-7-(3,4,5-trifluorophenyl)-7H-pyrrolo[2,3-d]pyrimidine

To a solution of 4-chloro-5-iodo-7H-pyrrolo[2,3-d]pyrimidine (2.6 g, 9.3 mmol) in DCM (50 mL) were added (3,4,5-trifluorophenyl)boronic acid (4.1 g, 23 mmol), Cu(OAc)₂ (4.2 g, 23 mmol), pyridine (4.4 mL, 56 mmol). The resulting mixture was stirred at room temperature under O₂ atmosphere overnight. NH3-H₂O (80 mL) was added and the reaction was filtered, the filtrate was partitioned between DCM and water, organic layer was separated, the aqueous phase was extracted with DCM twice, the combined organic layers were washed with brine, dried over Na₂SO₄, filtered and concentrated, the residue was purified by flash chromatography (silica gel, 0-30% EtOAc in petroleum ether) to afford 4-chloro-5-iodo-7-(3,4,5-trifluorophenyl)-7H-pyrrolo[2,3-d]pyrimidine (2.5 g, 66%) as a white solid. LC/MS ESI (m/z): 410 (M+H)⁺

### Step 2. tert-Butyl (R)-4-(5-iodo-7-(3,4,5-trifluorophenyl)-7H-pyrrolo[2,3-d]pyrimidin-4-yl)-2-methylpiperazine-1-carboxylate

To a solution of 4-chloro-5-iodo-7-(3,4,5-trifluorophenyl)-7H-pyrrolo[2,3-d]pyrimidine (600 mg, 1.5 mmol) in EtOH (5 mL) were added tert-butyl (R)-2-methylpiperazine-1-carboxylate (590 mg, 3.0 mmol) and DIPEA (1.5 mg, 8.9 mmol). The resulting mixture was heated to 100° C. overnight. After being cooled down to room temperature, solvent was removed and the residue was purified by flash column chromatography (silica gel, 0˜30%, ethyl acetate in petroleum ether) to afford tert-butyl (R)-4-(5-iodo-7-(3,4,5-trifluorophenyl)-7H-pyrrolo[2,3-d]pyrimidin-4-yl)-2-methylpiperazine-1-carboxylate (600 mg, 71%) as a white solid. LC/MS ESI (m/z): 574 (M+H)⁺.

### Step 3 tert-Butyl (R)-2-methyl-4-(5-(2-oxopyrrolidin-1-yl)-7-(3,4,5-trifluorophenyl)-7H-pyrrolo[2,3-d]pyrimidin-4-yl)piperazine-1-carboxylate

To a solution of tert-butyl (R)-4-(5-iodo-7-(3,4,5-trifluorophenyl)-7H-pyrrolo[2,3-d]pyrimidin-4-yl)-2-methylpiperazine-1-carboxylate (100 mg, 0.17 mmol) in DMF (5 mL) were added pyrrolidin-2-one (29 mg, 0.34 mmol), trans-cyclohexane-1,2-diamine (5.9 mg, 0.050 mmol), CuI (9.5 mg, 0.050 mmol) and K₃PO₄ (220 mg, 0.51 mmol). The resulting mixture was heated to 100° C. overnight. After being cooled down to room temperature. The reaction was quenched with water, extracted with EtOAc twice, the combined organic layers were washed with brine, dried over Na₂SO₄, filtered and concentrated. The residue was purified by flash column chromatography (silica gel, 0˜30%, ethyl acetate in petroleum ether) to give crude product which was further purified by prep-HPLC to afford tert-butyl (R)-2-methyl-4-(5-(2-oxopyrrolidin-1-yl)-7-(3,4,5-trifluorophenyl)-7H-pyrrolo[2,3-d]pyrimidin-4-yl)piperazine-1-carboxylate (19 mg, 20%) as a white solid. LC/MS ESI (m/z): 531 (M+H)⁺. ¹H NMR (400 MHz, CD₃OD) δ 8.32 (s, 1H), 7.75-7.69 (m, 2H), 7.67 (s, 1H), 4.34-4.28 (m, 1H), 4.23-4.17 (m, 1H), 4.02-3.96 (m, 2H), 3.91-3.85 (m, 1H), 3.81-3.74 (m, 1H), 3.49-3.44 (m, 1H), 3.35 (d, J=3.2 Hz, 1H), 3.23-3.15 (m, 1H), 2.65-2.60 (m, 2H), 2.29 (dt, J=11.4, 7.0 Hz, 2H), 1.47 (s, 9H), 1.08 (d, J=6.8 Hz, 3H).

The following compounds were prepared by the procedure analogous to the synthesis of compound 313 from the corresponding amide (pyrrolidin-2-one) or amine (piperidine).

| Cmpd | | |
|---|---|---|
| No. | Chemical Name | LCMS and ¹H NMR |
| 287 | tert-butyl (R)-4-(7-(3- | LC/MS ESI (m/z): 511 (M + H)⁺. ¹H NMR (400 MHz, |
| | chlorophenyl)-5-(piperidin-1- | CD₃OD) δ 8.21 (s, 1H), 7.83 (t, J = 2.0 Hz, 1H), 7.65-7.62 |
| | yl)-7H-pyrrolo[2,3-d]pyrimidin- | (m, 1H), 7.49 (t, J = 8.1 Hz, 1H), 7.38 - 7.33 (m, 1H), 7.05 |
| | 4-yl)-2-methylpiperazine-1- | (s, 1H), 4.91 (s, 1H), 4.68 (d, J = 13.6 Hz, 1H), 4.36-4.30 |
| | carboxylate | (m, 1H), 3.89 (d, J = 13.5 Hz, 1H), 3.41 (td, J = 14.1, 3.5 |
| | | Hz, 3H), 3.10-3.03 (m, 2H), 2.98-2.70 (m, 2H), 1.86-1.72 |
| | | (m, 4H), 1.64 (m, 2H), 1.48 (s, 9H), 1.03 (d, J = 6.8 |
| | | Hz, 3H). |
| 309 | tert-butyl (S)-4-(7-(3- | LC/MS ESI (m/z): 511.2 (M + H)⁺. ¹H NMR (400 MHz, |
| | chlorophenyl)-5-(2- | CD₃OD) δ 8.33 (s, 1H), 7.85 (t, J = 2.0 Hz, 1H), 7.68-7.64 |
| | oxopyrrolidin-1-yl)-7H- | (m, 2H), 7.53 (t, J = 8.1 Hz, 1H), 7.45-7.40 (m, 1H), 4.56 |
| | pyrrolo[2,3-d]pyrimidin-4-yl)-3- | (br, 1H), 4.07-3.96 (m, 2H), 3.89 (d, J = 13.2 Hz, 1H), |
| | methylpiperazine-1-carboxylate | 3.84-3.75 (m, 2H), 33.48 (td, J = 13.4, 3.4 Hz, 1H), |
| | | 3.29-3.20 (m, 1H), 3.13-2.99 (m, 1H), 2.67-2.61 (m, 2H), |
| | | 2.33-2.25 (m, 2H), 1.49 (s, 9H), 1.22 (d, J = 6.7 Hz, 3H). |
| 310 | tert-butyl (S)-4-(7-(3- | LC/MS ESI (m/z): 511.2 (M + H)⁺. ¹H NMR (400 MHz, |
| | chlorophenyl)-5-(piperidin-1- | CD₃OD) δ 8.22 (s, 1H), 7.83 (t, J = 1.9 Hz, 1H), 7.63 (dd, |
| | yl)-7H-pyrrolo[2,3-d]pyrimidin- | J = 8.1, 1.1 Hz, 1H), 7.48 (t, J = 8.1 Hz, 1H), 7.37-7.32 |
| | 4-yl)-3-methylpiperazine-1- | (m, 1H), 7.05 (s, 1H), 5.20 (br, 1H), 4.33 (br, 1H), 4.09 (d, |
| | carboxylate | J = 12.8 Hz, 1H), 3.92 (d, J = 13.1 Hz, 1H), 3.50-3.36 (m, |
| | | 2H), 3.13-2.93 (m, 3H), 2.91-2.70 (m, 2H), 1.85-1.72 |
| | | (m, 4H), 1.59 (br, 2H), 1.50 (s, 9H), 1.12 (d, J = 6.5 Hz, |
| | | 3H). |

### Example 70. Synthesis of tert-butyl (S)-4-(5-cyclopropyl-7-(3,5-difluorophenyl)-7H-pyrrolo[2,3-d]pyrimidin-4-yl)-2-(difluoromethyl)piperazine-1-carboxylate (Compound 314) and tert-butyl (R)-4-(5-cyclopropyl-7-(3,5-difluorophenyl)-7H-pyrrolo[2,3-d]pyrimidin-4-yl)-2-(difluoromethyl)piperazine-1-carboxylate (Compound 315)

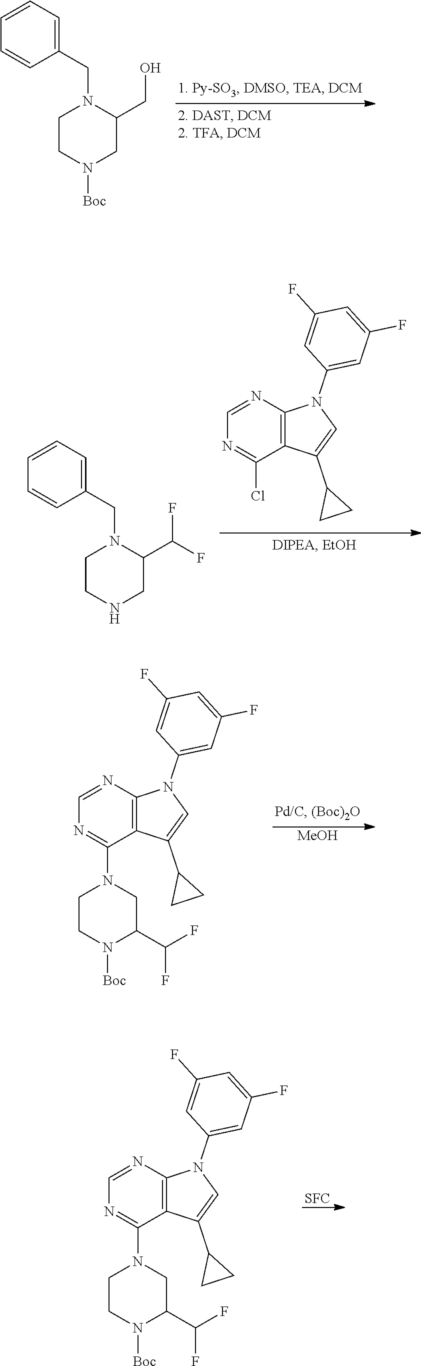

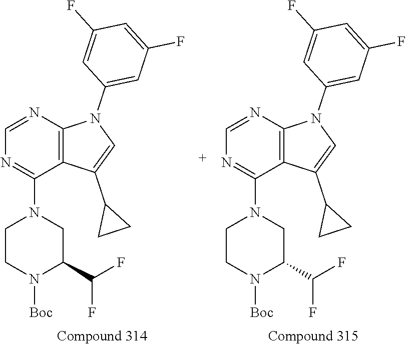 [SMILES: CC(C)(C)OC(=O)N1CCN(c2ncnc3c2c(C2CC2)cn3-c2cc(F)cc(F)c2)C[C@H]1C(F)F ; CC(C)(C)OC(=O)N1CCN(c2ncnc3c2c(C2CC2)cn3-c2cc(F)cc(F)c2)C[C@@H]1C(F)F]

### Step 1. tert-Butyl 4-benzyl-3-formylpiperazine-1-carboxylate

At 0° C., to a solution of tert-butyl 4-benzyl-3-(hydroxymethyl)piperazine-1-carboxylate (1.8 g, 5.9 mmol) in DCM (30 mL) were added DMSO (20 mL) was added TEA (4.1 mL, 29 mmol), followed by pyridine sulfur trioxide (4.7 g, 29 mmol) dropwise. The resulting mixture was stirred at room temperature overnight. The reaction was quenched with water, extracted with DCM twice, the combined organic layers were washed with brine, dried over Na₂SO₄, filtered and concentrated. The residue was purified by flash column chromatography (silica gel, 0˜60%, ethyl acetate in petroleum ether) to give tert-butyl 4-benzyl-3-formylpiperazine-1-carboxylate (900 mg, 50% yield) as an oil. LC/MS ESI (m/z): 305 (M+H)⁺.

### Step 2. tert-Butyl 4-benzyl-3-(difluoromethyl)piperazine-1-carboxylate

At 0° C., to a solution of tert-butyl 4-benzyl-3-formylpiperazine-1-carboxylate (900 mg, 3.0 mmol) in DCM (10 mL) was added DAST (950 mg, 5.9 mmol) dropwise. The resulting mixture was stirred at room temperature overnight. The reaction was quenched with NaHCO₃ (aq.), extracted with DCM twice, the combined organic layers were washed with brine, dried over Na₂SO₄, filtered and concentrated. The residue was purified by flash column chromatography (silica gel, 0˜50%, ethyl acetate in petroleum ether) to give tert-butyl 4-benzyl-3-(difluoromethyl)piperazine-1-carboxylate (330 mg, 34% yield) as an oil. LC/MS ESI (m/z): 327 (M+H)⁺.

### Step 3. I-Benzyl-2-(difluoromethyl)piperazine

To a solution of tert-butyl 4-benzyl-3-(difluoromethyl)piperazine-1-carboxylate (330 mg, 1.0 mmol) in DCM (5 mL) was added TFA (2 mL). The resulting mixture was stirred at room temperature for 2 h. After removal of solvent, the residue was diluted with DCM, washed with NaHCO₃ (aq.), the organic layer was extracted with DCM twice, the combined organic layers were washed with brine, dried over Na₂SO₄, filtered and concentrated. The residue was used in the next step directly. LC/MS ESI (m/z): 227 (M+H)⁺.

### Step 4. 4-(4-Benzyl-3-(difluoromethyl)piperazin-1-yl)-5-cyclopropyl-7-(3,5-difluorophenyl)-7H-pyrrolo[2,3-d]pyrimidine

A mixture of 4-chloro-5-cyclopropyl-7-(3,5-difluorophenyl)-7H-pyrrolo[2,3-d]pyrimidine (100 mg, 0.33 mmol, prepared following the procedure outlined in compound 256), 1-benzyl-2-(difluoromethyl)piperazine (150 mg, 0.66 mmol) and DIPEA (260 mg, 2.0 mmol) in EtOH (5 mL) was stirred at 100° C. overnight. After being cooled down to room temperature, solvent was removed. The residue was purified by flash chromatography (silica gel, 0˜50% EtOAc in petroleum ether) to afford 4-(4-benzyl-3-(difluoromethyl)piperazin-1-yl)-5-cyclopropyl-7-(3,5-difluorophenyl)-7H-pyrrolo[2,3-d]pyrimidine (100 mg, 61% yield) as a white solid. LC/MS ESI (m/z): 496 (M+H)⁺.

### Step 5. tert-Butyl 4-(5-cyclopropyl-7-(3,5-difluorophenyl)-7H-pyrrolo[2,3-d]pyrimidin-4-yl)-2-(difluoromethyl)piperazine-1-carboxylate

A mixture of 4-(4-benzyl-3-(difluoromethyl)piperazin-1-yl)-5-cyclopropyl-7-(3,5-difluorophenyl)-7H-pyrrolo[2,3-d]pyrimidine (100 mg, 0.20 mmol), (Boc)₂O (44 mg, 0.40 mmol) and Pd/C (30 mg, wetted with ca. 55% water) in MeOH (8 mL) was stirred at 40° C. overnight, under H₂ atmosphere (˜1 atm). After being cooled down to room temperature, the reaction was filtered, filtrate was partitioned between EtOAc and water, organic layer was separated, the aqueous phase was extracted with EtOAc twice, dried over Na₂SO₄, filtered and concentrated, the residue was purified by flash column chromatography (silica gel, 0˜50% EtOAc in petroleum ether) to give tert-butyl 4-(5-cyclopropyl-7-(3,5-difluorophenyl)-7H-pyrrolo[2,3-d]pyrimidin-4-yl)-2-(difluoromethyl)piperazine-1-carboxylate (93 mg, 91% yield), as a white solid. LCMS ESI (m/z): 506 (M+H)⁺.

### Step 6 tert-Butyl (S)-4-(5-cyclopropyl-7-(3,5-difluorophenyl)-7H-pyrrolo[2,3-d]pyrimidin-4-yl)-2-(difluoromethyl)piperazine-1-carboxylate and tert-butyl (R)-4-(5-cyclopropyl-7-(3,5-difluorophenyl)-7H-pyrrolo[2,3-d]pyrimidin-4-yl)-2-(difluoromethyl)piperazine-1-carboxylate

tert-butyl 4-(5-cyclopropyl-7-(3,5-difluorophenyl)-7H-pyrrolo[2,3-d]pyrimidin-4-yl)-2-(difluoromethyl)piperazine-1-carboxylate was separated by SFC to give two isomers:

Peak 1: shorter retention time, labeled as compound 314 (tert-butyl (S)-4-(5-cyclopropyl-7-(3,5-difluorophenyl)-7H-pyrrolo[2,3-d]pyrimidin-4-yl)-2-(difluoromethyl)piperazine-1-carboxylate), LCMS ESI (m/z): 506 (M+H)⁺. ¹H NMR (400 MHz, CD₃OD) δ 8.38 (s, 1H), 7.58-7.52 (m, 2H), 7.34 (d, J=0.6 Hz, 1H), 6.98-6.90 (m, 1H), 6.16 (m, J=56.1, 5.8 Hz, 1H), 4.52 (d, J=11.1 Hz, 2H), 4.38 (d, J=14.0 Hz, 1H), 4.10 (d, J=13.3 Hz, 1H), 3.49-3.36 (m, 2H), 3.16-3.08 (m, 1H), 2.09-2.03 (m, 1H), 1.49 (s, 9H), 1.07-0.97 (m, 3H), 0.72-0.65 (m, 1H).

Peak 2: longer retention time, labeled as compound 315 (tert-butyl (R)-4-(5-cyclopropyl-7-(3,5-difluorophenyl)-7H-pyrrolo[2,3-d]pyrimidin-4-yl)-2-(difluoromethyl)piperazine-1-carboxylate), LCMS ESI (m/z): 506 (M+H)⁺. ¹H NMR (400 MHz, CD₃OD) δ 8.38 (s, 1H), 7.58-7.52 (m, 2H), 7.34 (d, J=0.7 Hz, 1H), 6.97-6.91 (m, 1H), 6.31-6.00 (m, 1H), 4.52 (d, J=10.7 Hz, 2H), 4.38 (d, J=13.4 Hz, 1H), 4.10 (d, J=13.2 Hz, 1H), 3.48-3.36 (m, 2H), 3.16-3.09 (m, 1H), 2.09-2.03 (m, 1H), 1.49 (s, 9H), 1.08-0.97 (m, 3H), 0.71-0.65 (m, 1H).

Preparative separation method: Instrument: Waters Thar 80 preparative SFC; Column: ChiralPak AD, 250×21.2 mm I.D., 5 μm; Mobile phase: A for CO2 and B for MEOH+0.1% NH3H2O; Gradient: B 30%; Flow rate: 50 mL/min; Back pressure: 100 bar; Column temperature: 35° C.; Wavelength: 240 nm; Cycle-time: 3 min; Elution time: 2 h.

### Example 71. Synthesis of tert-butyl (S)-4-(7-(3-chlorophenyl)-5-((S)-2-methylpyrrolidin-1-yl)-7H-pyrrolo[2,3-d]pyrimidin-4-yl)-3-methylpiperazine-1-carboxylate (Compound 316)

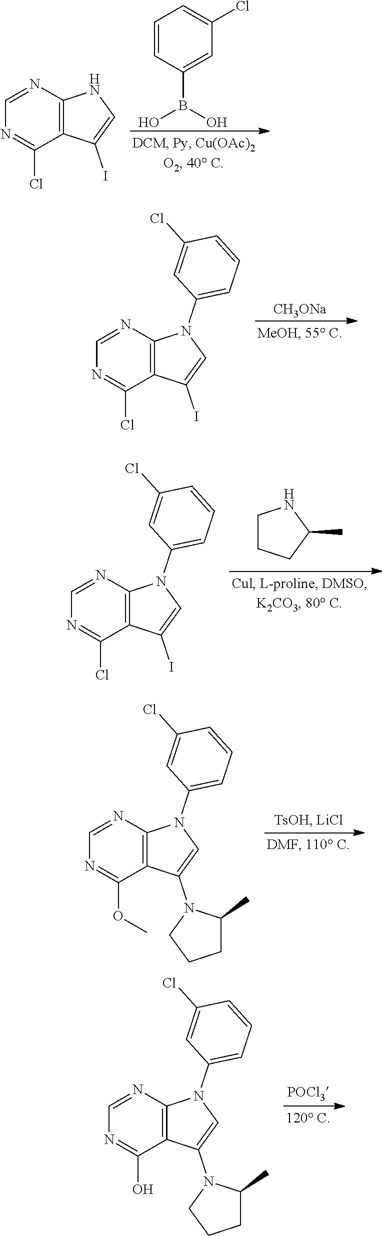

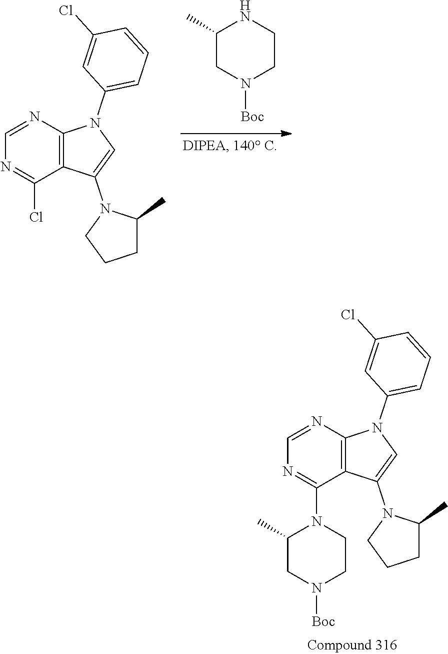

### Step 1. 4-Chloro-7-(3-chlorophenyl)-5-iodo-7H-pyrrolo[2,3-d]pyrimidine

To a solution of 4-chloro-5-iodo-7H-pyrrolo[2,3-d]pyrimidine (10 g, 36 mmol) in DCM (40 mL) were added (3-chlorophenyl)boronic acid (11 g, 72 mmol), 4 A molecular sieves (8 g), Cu(OAc)₂ (26 g, 140 mmol) and pyridine (17 g, 210 mmol). The resulting mixture was stirred at 40° C. under O₂ atmosphere for 2 days. The reaction was quenched with NH₄OH (50 mL) at ice water, filtered, the filtrate was extract with DCM twice, the combined organic layers were dried over Na₂SO₄, filtered and concentrated, the residue was purified by flash column chromatography (silica gel, 0-30% EtOAc in petroleum ether) to afford 4-chloro-7-(3-chlorophenyl)-5-iodo-7H-pyrrolo[2,3-d]pyrimidine (8.8 g, 67%) as a solid. LC/MS ESI (m/z): 390 (M+H)⁺.

### Step 2. 7-(3-Chlorophenyl)-5-iodo-4-methoxy-7H-pyrrolo[2,3-d]pyrimidine

To a solution of 4-chloro-7-(3-chlorophenyl)-5-iodo-7H-pyrrolo[2,3-d]pyrimidine (5.0 g, 13 mmol) in MeOH (20 m1) was added CH₃ONa (77 ml, 5.0M in MeOH) and the mixture was stirred at 55° C. for 4 hours under N₂. The reaction was quenched with ice water and it was extracted with DCM twice, the combined organic layers were dried over Na₂SO₄, filtered and concentrated, the residue was purified by flash column chromatography (silica gel, 0˜30% EtOAc in petroleum ether) to afford 7-(3-chlorophenyl)-5-iodo-4-methoxy-7H-pyrrolo[2,3-d]pyrimidine (3.8 g, 78%) as a solid. LC/MS ESI (m/z): 386 (M+H)⁺

### Step 3. (S)-7-(3-Chlorophenyl)-4-methoxy-5-(2-methylpyrrolidin-1-yl)-7H-pyrrolo[2,3-d]pyrimidine

To the solution of 7-(3-chlorophenyl)-5-iodo-4-methoxy-7H-pyrrolo[2,3-d]pyrimidine (1000 mg, 2.6 mmol) in DMSO (6 mL) were added CuI (98 mg, 0.46 mmol), L-proline (120 mg, 1.0 mmol), K₂CO₃ (1100 mg, 7.8 mmol) and (S)-2-methylpyrrolidine (880 mg, 10 mmol). The resulting reaction mixture was stirred at 80° C. under N₂ overnight. After which the reaction was extracted with EtOAc and water. The combined organic layers were dried over Na₂SO₄, filtered, and concentrated under reduced pressure. The crude product was purified by flash column chromatography (silica gel, 0˜30%, EtOAc in petroleum ether) to afford (S)-7-(3-chlorophenyl)-4-methoxy-5-(2-methylpyrrolidin-1-yl)-7H-pyrrolo[2,3-d]pyrimidine (100 mg, 11%) as a solid. LC/MS ESI (m/z): 343 (M+H)⁺.

### Step 4. (S)-7-(3-Chlorophenyl)-5-(2-methylpyrrolidin-1-yl)-7H-pyrrolo[2,3-d]pyrimidin-4-ol

To a solution of (S)-7-(3-chlorophenyl)-4-methoxy-5-(2-methylpyrrolidin-1-yl)-7H-pyrrolo[2,3-d]pyrimidine (100 mg, 0.29 mmol) in DMF (4 mL) were added p-Toluenesulfonic acid (550 mg, 2.9 mmol) and LiCl (120 mg, 2.9 mmol). The resulting mixture was heated to 110° C. for 2 hours. After being cooled down to room temperature. The reaction was quenched with water, extracted with EtOAc twice, the combined organic layers were washed with brine, dried over Na₂SO₄, filtered and concentrated to afford (S)-7-(3-chlorophenyl)-5-(2-methylpyrrolidin-1-yl)-7H-pyrrolo[2,3-d]pyrimidin-4-ol (80 mg, 84%) as a solid. LC/MS ESI (m/z): 329 (M+H)⁺.

### Step 5. (S)-4-Chloro-7-(3-chlorophenyl)-5-(2-methylpyrrolidin-1-yl)-7H-pyrrolo[2,3-d]pyrimidine

To a solution of (S)-7-(3-chlorophenyl)-5-(2-methylpyrrolidin-1-yl)-7H-pyrrolo[2,3-d]pyrimidin-4-ol (40 mg, 0.12 mmol) in POCl₃ (4 mL). The resulting mixture was heated to 120° C. overnight, the reaction was concentrated. The reaction was quenched with NaHCO₃ (aq.), extracted with DCM twice, the combined organic layers were washed with brine, dried over Na₂SO₄, filtered and concentrated to afford (S)-4-chloro-7-(3-chlorophenyl)-5-(2-methylpyrrolidin-1-yl)-7H-pyrrolo[2,3-d]pyrimidine (34 mg, 81%) as a yellow solid. LC/MS ESI (m/z): 347 (M+H)⁺.

### Step 6. tert-Butyl (S)-4-(7-(3-chlorophenyl)-5-((S)-2-methylpyrrolidin-1-yl)-7H-pyrrolo[2,3-d]pyrimidin-4-yl)-3-methylpiperazine-1-carboxylate

To a solution of (S)-4-chloro-7-(3-chlorophenyl)-5-(2-methylpyrrolidin-1-yl)-7H-pyrrolo[2,3-d]pyrimidine (17 mg, 0.050 mmol) in DIPEA (1 mL) was added tert-butyl (S)-3-methylpiperazine-1-carboxylate (20 mg, 0.10 mmol). The resulting mixture was heated to 140° C. for 3 hours. After being cooled down to room temperature, solvent was removed and the residue was purified by flash column chromatography (silica gel, 0˜30%, ethyl acetate in petroleum ether) to give crude product which was further purified by prep-HPLC to afford tert-butyl (S)-4-(7-(3-chlorophenyl)-5-((S)-2-methylpyrrolidin-1l-yl)-7H-pyrrolo[2,3-d]pyrimidin-4-yl)-3-methylpiperazine-1-carboxylate (16 mg, 600%) as a white solid. LC/MS ESI (m/z): 511. ¹H NMR (400 MHz, CD₃CD) 68.24 (s, 1H), 7.86 (t, J=2.0 Hz, 1H), 7.67-7.64 (m, 1H), 7.49 (t, J=8.1 Hz, 1H), 7.37-7.34 (m, 1H), 7.09 (s, 1H), 5.35 (s, 1H), 4.12 (d, J=11.7 Hz, 1H), 4.02-3.87 (m, 2H), 3.70 (s, 1H), 3.54-3.36 (m, 3H), 3.01 (s, 1H), 2.69 (d, J=7.9 Hz, 1H), 2.26-2.18 (m, 1H), 2.03-1.95 (m, 1H), 1.92-1.83 (m, 1H), 1.70-1.62 (m, 1H), 1.50 (s, 9H), 1.15 (d, J=5.4 Hz, 3H), 1.01 (d, J=6.1 Hz, 3H).

The following compounds were prepared by the procedures analogous to the synthesis of compound 316 from the corresponding amines.

| Cmpd | | |
|---|---|---|
| No. | Chemical Name | LCMS and ¹H NMR |
| 286 | tert-butyl (S)-4-(7-(3- | ¹H NMR (400 MHz, CDCl₃) δ 8.37 (s, 1H), 7.69 (s, 1H), |
| | chlorophenyl)-5-((R)-2- | 7.64 (d, J = 8.0 Hz, 1H), 7.43 (t, J = 8.0 Hz, 1H), 7.32-7.27 |
| | methylpyrrolidin-1-yl)-7H- | (m, 1H), 6.81 (s, 1H), 5.16-4.76 (m, 2H), 4.08 (d, J = |
| | pyrrolo[2,3-d]pyrimidin-4-yl)-3- | 130.9 Hz, 3H), 3.60-3.50 (m, 1H), 3.31-3.21 (m, 2H), |
| | methylpiperazine-1-carboxylate | 3.16-3.09 (m, 1H), 2.67-2.58 (m, 1H), 2.21-2.15 (m, |
| | | 1H), 1.97-1.91 (m, 1H), 1.88-1.82 (m, 1H), 1.67-1.62 |
| | | (m, 1H), 1.50 (s, 9H), 1.34 (d, J = 5.1 Hz, 3H), 1.14 (d, |
| | | J = 6.0 Hz, 3H). |
| 255 | tert-butyl (R)-4-(7-(3,5- | ¹H NMR (400 MHz, CDCl₃) δ 8.39 (s, 1H), 7.42 (dd, J = |
| | difluorophenyl)-5-(pyrrolidin-1- | 8.4, 2.1 Hz, 2H), 6.76-6.70 (m, 1H), 6.69 (s, 1H), 4.97 (s, |
| | yl)-7H-pyrrolo[2,3-d]pyrimidin- | 1H), 4.25-4.07 (m, 2H), 3.98-3.85 (m, 1H), 3.39 (t, J = |
| | 4-yl)-3-methylpiperazine-1- | 11.7 Hz, 1H), 3.25 (s, 1H), 3.16 (s, 2H), 2.98-2.85 (m, |
| | carboxylate | 3H), 1.98 (s, 4H), 1.49 (s, 9H), 1.13 (s, 3H). |
| 253 | tert-butyl (S)-4-(7-(3,5- | ¹H NMR (400 MHz, CDCl₃) δ 8.39 (s, 1H), 7.42 (dd, J = |
| | difluorophenyl)-5-(pyrrolidin-1- | 8.5, 2.2 Hz, 2H), 6.76-6.70 (m, 1H), 6.69 (s, 1H), |
| | yl)-7H-pyrrolo[2,3-d]pyrimidin- | 5.02-4.92 (m, 1H), 4.23-4.04 (m, 2H), 3.98-3.84 (m, 1H), |
| | 4-yl)-3-methylpiperazine-1- | 3.39 (t, J = 11.5 Hz, 1H), 3.33-3.22 (m, 1H), 3.16 (s, 2H), |
| | carboxylate | 2.98-2.83 (m, 3H), 2.00-1.96 (m, 4H), 1.49 (s, 9H), |
| | | 1.16-1.10 (m, 3H). |
| 262 | tert-butyl (R)-4-(7-(3- | ¹H NMR (400 MHz, CDCl₃) δ 8.38 (s, 1H), 7.73 (t, J = 2.0 |
| | chlorophenyl)-5-((S)-2- | Hz, 1H), 7.69-7.65 (m, 1H), 7.42 (t, J = 8.1 Hz, 1H), |
| | methylpyrrolidin-1-yl)-7H- | 7.31-7.27 (m, 1H), 6.76 (s, 1H), 4.90 (d, J = 12.3 Hz, 1H), |
| | pyrrolo[2,3-d]pyrimidin-4-yl)-2- | 4.40 (d, 2H), 3.93 (d, J = 12.9 Hz, 1H), 3.64 (s, 1H), |
| | methylpiperazine-1-carboxylate | 3.44-3.33 (m, 2H), 3.23 (d, J = 10.8 Hz, 1H), 2.94-2.84 |
| | | (m, 1H), 2.72-2.62 (m, 1H), 2.23-2.17 (m, 1H), 2.01-1.93 |
| | | (m, 1H), 1.90-1.82 (m, 1H), 1.68-1.64 (m, 1H), 1.50 (s, 9H), |
| | | 1.15 (d, J = 4.8 Hz, 3H), 1.11 (d, J = 6.7 Hz, 3H). |
| 244 | tert-butyl (S)-4-(7-(3,5- | ¹H NMR (400 MHz, CDCl₃) δ 8.38 (s, 1H), 7.42 (dd, J = |
| | difluorophenyl)-5-(pyrrolidin-1- | 8.4, 2.1 Hz, 2H), 6.76-6.69 (m, 2H), 4.77 (d, J = 12.1 Hz, |
| | yl)-7H-pyrrolo[2,3-d]pyrimidin- | 1H), 4.38 (d, J = 12.9 Hz, 2H), 3.92 (d, J = 13.7 Hz, 1H), |
| | 4-yl)-2-methylpiperazine-1- | 3.36-3.29 (m, 1H), 3.26-3.19 (m, 3H), 3.01-2.93 (m, |
| | carboxylate | 1H), 2.88-2.82 (m, 2H), 1.99 (t, J = 6.0 Hz, 4H), 1.49 (s, |
| | | 9H), 1.10 (d, J = 6.8 Hz, 3H). |
| 245 | tert-butyl (R)-4-(7-(3,5- | ¹H NMR (400 MHz, CDCl₃) δ 8.38 (s, 1H), 7.42 (dd, J = |
| | difluorophenyl)-5-(pyrrolidin-1- | 8.5, 2.2 Hz, 2H), 6.76-6.69 (m, 2H), 4.77 (d, J = 12.3 Hz, |
| | yl)-7H-pyrrolo[2,3-d]pyrimidin- | 1H), 4.39 (d, J = 13.3 Hz, 2H), 3.92 (d, J = 13.4 Hz, 1H), |
| | 4-yl)-2-methylpiperazine-1- | 3.36-3.29 (m, 1H), 3.27-3.20 (m, 3H), 3.00-2.93 (m, |
| | carboxylate | 1H), 2.87-2.82 (m, 2H), 2.01-1.97 (m, 4H), 1.49 (s, 9H), |
| | | 1.10 (d, J = 6.8 Hz, 3H). |
| 299 | tert-butyl (R)-4-(7-(3- | ¹H NMR (400 MHz, CDCl₃) δ 8.31 (s, 1H), 7.69 (s, 1H), |
| | chlorophenyl)-5-((R)-2- | 7.65 (d, J = 8.0 Hz, 1H), 7.42 (t, J = 8.1 Hz, 1H), |
| | methylpyrrolidin-1-yl)-7H- | 7.32-7.26 (m, 1H), 6.83 (s, 1H), 4.97-4.69 (m, 2H), |
| | pyrrolo[2,3-d]pyrimidin-4-yl)-2- | 4.38-4.29 (m, 1H), 3.95-3.87 (m, 1H), 3.46-3.38 (m, 1H), |
| | methylpiperazine-1-carboxylate | 3.33-3.18 (m, 3H), 3.12-3.03 (m, 1H), 2.77-2.69 (m, 1H), |
| | | 2.20-2.12 (m, 1H), 1.95-1.82 (m, 2H), 1.66-1.60 (m, |
| | | 1H), 1.50 (d, J = 3.4 Hz, 9H), 1.23 (d, J = 6.7 Hz, 3H), |
| | | 1.13 (d, J = 6.0 Hz, 3H). |

### Example 72. Synthesis of tert-butyl (S)-3-methyl-4-(5-(1-methyl-1H-pyrazol-4-yl)-7-(3-(trifluoromethoxy)phenyl)-7H-pyrrolo[2,3-d]pyrimidin-4-yl)piperazine-1-carboxylate (Compound 321)

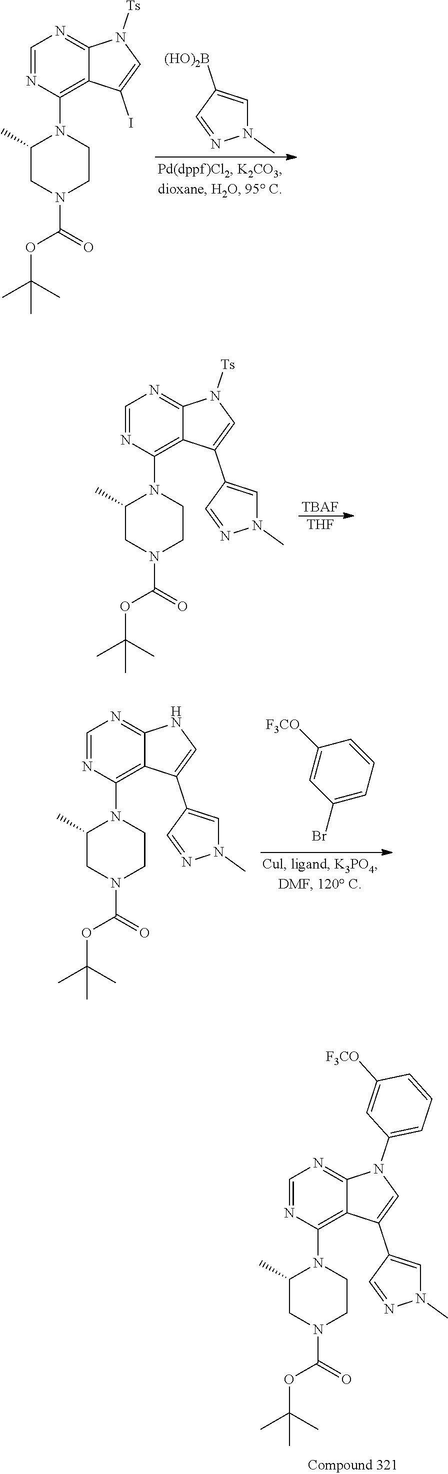

### Step 1. tert-Butyl (S)-3-methyl-4-(5-(I-methyl-1H-pyrazol-4-yl)-7-tosyl-7H-pyrrolo[2,3-d]pyrimidin-4-yl)piperazine-1-carboxylate

A mixture of tert-butyl (S)-4-(5-iodo-7-tosyl-7H-pyrrolo[2,3-d]pyrimidin-4-yl)-3-methylpiperazine-1-carboxylate (1.5 g, 2.5 mmol), (1-methyl-1H-pyrazol-4-yl)boronic acid (630 mg, 5.0 mmol), Pd(dppf)Cl₂ (180 mg, 0.30 mmol) and K₂CO₃ (1000 mg, 7.5 mmol) in dioxane (20 mL) and H₂O (5 mL) was stirred at 95° C. for 12 hrs. The reaction mixture was concentrated. The residue was purified by column chromatography on silica gel (0˜50% EtOAc in petroleum ether) to give the tert-butyl (S)-3-methyl-4-(5-(1-methyl-1H-pyrazol-4-yl)-7-tosyl-7H-pyrrolo[2,3-d]pyrimidin-4-yl)piperazine-1-carboxylate (1.3 g, 91%) as a yellow solid. LC/MS ESI (m/z): 552 (M+H)⁺.

### Step 2. tert-Butyl (S)-3-methyl-4-(5-(I-methyl-1H-pyrazol-4-yl)-7H-pyrrolo[2,3-d]pyrimidin-4-yl)piperazine-1-carboxylate

To a solution of tert-butyl (S)-3-methyl-4-(5-(1-methyl-1H-pyrazol-4-yl)-7-tosyl-7H-pyrrolo[2,3-d]pyrimidin-4-yl)piperazine-1-carboxylate (1.3 g, 2.3 mmol) in THE (10 mL) was added TBAF (9.1 mL, 1.0M in THF). The resulting mixture was stirred at room temperature for 2 hrs. The reaction mixture diluted with water and extracted with EtOAc (100 m1×2). The combined layers were washed with brine, dried over Na₂SO₄ and concentrated. The residue was purified by column chromatography on silica gel (0˜80% EtOAc in petroleum ether) to give the tert-butyl (S)-3-methyl-4-(5-(1-methyl-1H-pyrazol-4-yl)-7H-pyrrolo[2,3-d]pyrimidin-4-yl)piperazine-1-carboxylate (850 mg, 2.1 mmol, 94%) as a yellow solid. LC/MS ESI (m/z): 398 (M+H)⁺.

### Step 3. tert-Butyl (S)-3-methyl-4-(5-(I-methyl-1H-pyrazol-4-yl)-7-(3-(trifluoromethoxy)phenyl)-7H-pyrrolo[2,3-d]pyrimidin-4-yl)piperazine-1-carboxylate

A mixture of tert-butyl (S)-3-methyl-4-(5-(1-methyl-1H-pyrazol-4-yl)-7H-pyrrolo[2,3-d]pyrimidin-4-yl)piperazine-1-carboxylate (150 mg, 0.38 mmol), 1-bromo-3-(trifluoromethoxy)benzene (180 mg, 0.76 mmol), CuI (36 mg, 0.19 mmol), trans-1,2-diaminocyclohexane (13 mg, 0.11 mmol) and K₃PO₄ (240 mg, 1.1 mmol) in DMF (10 mL) was heated at 120° C. for 12 hours. The reaction mixture was concentrated. The residue was purified by column chromatography on silica gel (0-50% EtOAc in petroleum ether, V/V) to give the crude product. The crude product was purified by prep-HPLC to give the tert-butyl (S)-3-methyl-4-(5-(1-methyl-1H-pyrazol-4-yl)-7-(3-(trifluoromethoxy)phenyl)-7H-pyrrolo[2,3-d]pyrimidin-4-yl)piperazine-1-carboxylate (80 mg, 0.14 mmol, 38%) as a white solid. LC/MS ESI (m/z): 558.6 (M+H)⁺. ¹H NMR (400 MHz, CD₃OD) δ 8.40 (s, 1H), 7.86 (d, J=8.9 Hz, 2H), 7.79 (d, J=8.2 Hz, 1H), 7.70 (br, 1H), 7.67-7.62 (m, 2H), 7.32 (d, J=8.3 Hz, 1H), 4.19 (br, 1H), 3.99 (s, 3H), 3.88 (d, J=12.8 Hz, 1H), 3.58 (t, J=15.0 Hz, 2H), 3.28-3.21 (m, 1H), 2.99 (br, 2H), 1.45 (s, 9H), 0.99 (d, J=6.6 Hz, 3H).

### Example 73. Synthesis of tert-butyl (R)-4-(7-(3-chlorophenyl)-5-((R)-2-(hydroxymethyl)pyrrolidin-1-yl)-7H-pyrrolo[2,3-d]pyrimidin-4-yl)-2-methylpiperazine-1-carboxylate (Compound 1002)

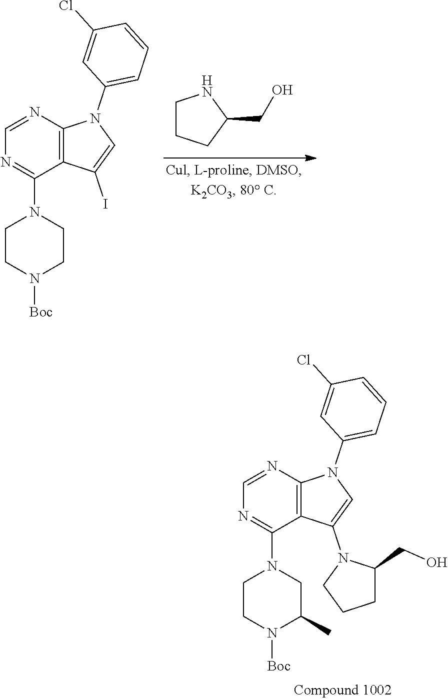

To the solution of tert-butyl (R)-4-(7-(3-chlorophenyl)-5-iodo-7H-pyrrolo[2,3-d]pyrimidin-4-yl)-2-methylpiperazine-1-carboxylate (400 mg, 0.72 mmol, prepared following the procedure outlined in compound 274) in DMSO (5 mL) were added Cu (28 mg, 0.14 mmol), L-proline (33 mg, 0.29 mmol), K₂CH₃ (140 mg, 2.2 mmol) and (R)-pyrrolidin-2-ylmethanol (290 mg, 2.9). The resulting reaction mixture was stirred at 80° C. under N₂ overnight. After it was extracted with EtOAc and water. The combined organic layers were dried over Na₂SO₄, filtered and concentrated under reduced pressure. The crude product was purified by flash column chromatography (silica gel, ˜179, methonal in DCM) to afford tert-butyl (R)-4-(7-(3-chlorophenyl)-5-((R)-2-(hydroxymethyl)pyrrolidin-1-yl)-7H-pyrrolo[2,3-d]pyrimidin-4-yl)-2-methylpiperazine-1-carboxylate (100 mg, 260) as brown solid. LC/MS ESI (m/z): 527 (M+H). ¹H NMR (400 m1 Hz, CD₃OD) δ 8.20 (s, 1H), 7.83 (t, J=2.0 Hz, 1H), 7.69-7.65 (m, 1H), 7.52-7.48 (m, 1H), 7.39-7.35 (m, 1H), 7.21 (s, 1H), 4.65-4.60 (m, 1H), 4.52-4.46 (m, 1H), 4.38-4.33 (m, 1H), 4.26-4.15 (m, 2H), 4.05-3.98 (m, 1H), 3.95-43.90 (m, 1H), 3.47 (dd, J=9.0, 4.5 Hz, 1H), 3.40-3.33 (m, 2H), 3.28-3.21 (m, 2H), 2.31-2.23 (m, 1H), 2.12-2.00 (m, 2H), 1.87-1.79 (m, 1H), 1.49 (s, 9H), 1.14 (d, J=6.7 Hz, 3H).

The following compounds were prepared by the procedures analogous to the synthesis of compound 1002 from the corresponding aryl halides.

| Cmpd | | |
|---|---|---|
| No. | Chemical Name | LCMS and ¹H NMR |
| 298 | tert-butyl (S)-4-(7-(3- | ¹H NMR (400 MHz, CD₃OD) δ 8.22 (s, 1H), 7.83 (t, J = 1.9 |
| | chlorophenyl)-5-((S)-2- | Hz, 1H), 7.67 (d, J = 8.2 Hz, 1H), 7.50 (t, J = 8.1 Hz, 1H), |
| | (hydroxymethyl)pyrrolidin-1- | 7.38-7.35 (m, 1H), 7.19 (s, 1H), 4.96 (s, 1H), 4.58 (s, 1H), |
| | yl)-7H-pyrrolo[2,3-d]pyrimidin- | 4.36-4.29 (m, 1H), 4.17-4.10 (m, 3H), 3.98-3.93 (m, |
| | 4-yl)-3-methylpiperazine-1- | 2H), 3.50-3.43 (m, 1H), 3.26-3.19 (m, 2H), 3.12-3.01 |
| | carboxylate | (m, 1H), 2.27-2.20 (m, 1H), 2.08-2.03 (m, 1H), 2.02-1.95 |
| | | (m, 1H), 1.80-1.73 (m, 1H), 1.50 (s, 9H), 1.27 (d, |
| | | J = 6.5 Hz, 3H). |
| 283 | tert-butyl (R)-4-(7-(3- | ¹H NMR (400 MHz, CDCl₃) δ 8.33 (s, 1H), 7.70 (t, J = 2.0 |
| | chlorophenyl)-5-((S)-2- | Hz, 1H), 7.64-7.60 (m, 1H), 7.41 (t, J = 8.1 Hz, 1H), |
| | (hydroxymethyl)pyrrolidin-1- | 7.29-7.27 (m, 1H), 6.72 (s, 1H), 4.70-4.64 (m, 1H), 4.51 |
| | yl)-7H-pyrrolo[2,3-d]pyrimidin- | (d, J = 13.2 Hz, 1H), 4.36 (s, 1H), 3.97-3.90 (m, 3H), |
| | 4-yl)-2-methylpiperazine-1- | 3.67-3.61 (m, 1H), 3.36-3.29 (m, 2H), 3.14 (td, J = 12.5, |
| | carboxylate | 3.4 Hz, 1H), 3.03 (t, J = 6.7 Hz, 2H), 2.04-1.99 (m, 1H), |
| | | 1.87-1.84 (m, 2H), 1.62-1.56 (m, 1H), 1.50 (s, 9H), 1.18 |
| | | (d, J = 6.7 Hz, 3H). |

### Example 74. Synthesis of 1,1,1-trifluoro-2-methylpropan-2-yl (S)-4-(7-cyclohexyl-5-cyclopropyl-7H-pyrrolo[2,3-d]pyrimidin-4-yl)-3-methylpiperazine-1-carboxylate (Compound 325)

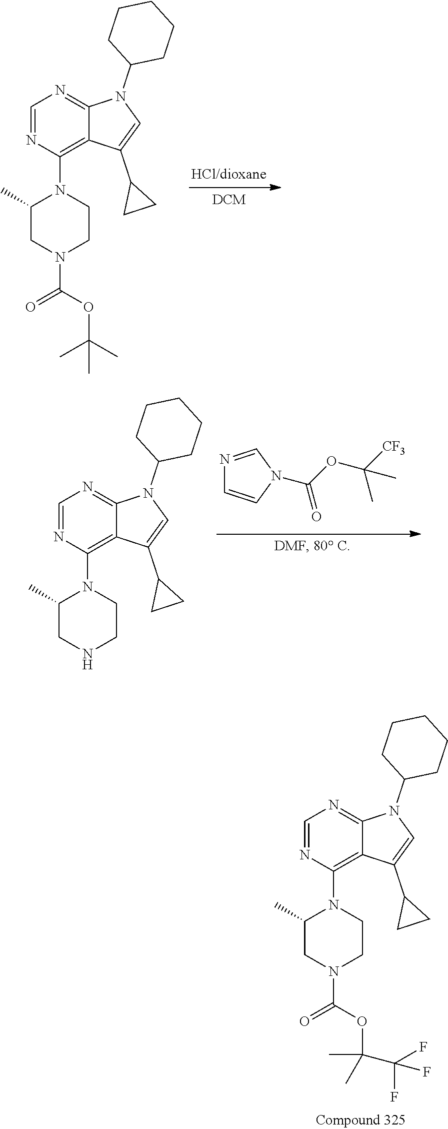

### Step 1. (S)-7-Cyclohexyl-5-cyclopropyl-4-(2-methylpiperazin-1-yl)-7H-pyrrolo[2,3-d]pyrimidine

To a solution of tert-butyl (S)-4-(7-cyclohexyl-5-cyclopropyl-7H-pyrrolo[2,3-d]pyrimidin-4-yl)-3-methylpiperazine-1-carboxylate (130 mg, 0.30 mmol, prepared following the procedure of compound 326, step 3) in DCM (1 mL) was added HCl/dioxane (1.0 mL, 4.0 M). The resulting mixture was stirred at room temperature overnight. After removal of solvent under reduced pressure, the residue was used in the next step directly. (S)-7-cyclohexyl-5-cyclopropyl-4-(2-methylpiperazin-1-yl)-7H-pyrrolo[2,3-d]pyrimidine (95 mg, 93%). LC/MS ESI (m/z): 340 (M+H)⁺.

### Step 2. 1,1,1-Trifluoro-2-methylpropan-2-yl (S)-4-(7-cyclohexyl-5-cyclopropyl-7H-pyrrolo[2,3-d]pyrimidin-4-yl)-3-methylpiperazine-1-carboxylate

To a solution of (S)-7-cyclohexyl-5-cyclopropyl-4-(2-methylpiperazin-1-yl)-7H-pyrrolo[2,3-d]pyrimidine (76 mg, 0.22 mmol) in DMF (0.5 mL) were added 1,1,1-trifluoro-2-methylpropan-2-yl 1H-imidazole-1-carboxylate (88 mg, 0.40 mmol, prepared following the procedure of compound 247, step 1). The reaction mixture was stirred at 80° C. overnight under N₂. The reaction was quenched with water, extracted with EtOAc twice, the combined organic layers were washed with brine, dried over Na₂SO₄, filtered and concentrated. The residue was purified by prep-HPLC to afford 1,1,1-trifluoro-2-methylpropan-2-yl (S)-4-(7-cyclohexyl-5-cyclopropyl-7H-pyrrolo[2,3-d]pyrimidin-4-yl)-3-methylpiperazine-1-carboxylate (20 mg, 20% yield) as a white solid. LC/MS ESI (m/z): 494 (M+H)⁺. ¹H NMR (400 MHz, MeOD) δ 8.31 (s, 1H), 7.29 (s, 1H), 4.95-4.86 (m, 1H), 4.72-4.58 (m, 1H), 4.28-4.01 (m, 2H), 4.01-3.84 (m, 1H), 3.75 (t, J=11.1 Hz, 1H), 3.57-3.37 (m, 1H), 3.26-3.08 (m, 1H), 2.04-1.88 (m, 5H), 1.88-1.75 (m, 3H), 1.72 (d, J=6.0 Hz, 6H), 1.52 (dd, J=24.8, 12.4 Hz, 2H), 1.41-1.32 (m, 1H), 1.30 (d, J=6.7 Hz, 3H), 1.10-0.94 (m, 2H), 0.89-0.79 (m, 1H), 0.79-0.67 (m, 1H). ¹⁹F NMR (377 MHz, MeOD) δ −85.03 (d, J=40.1 Hz).

### Example 75. Synthesis of tert-butyl (S)-4-(7-cyclohexyl-5-cyclopropyl-7H-pyrrolo[2,3-d]pyrimidin-4-yl)-3-methylpiperazine-1-carboxylate (Compound 326)

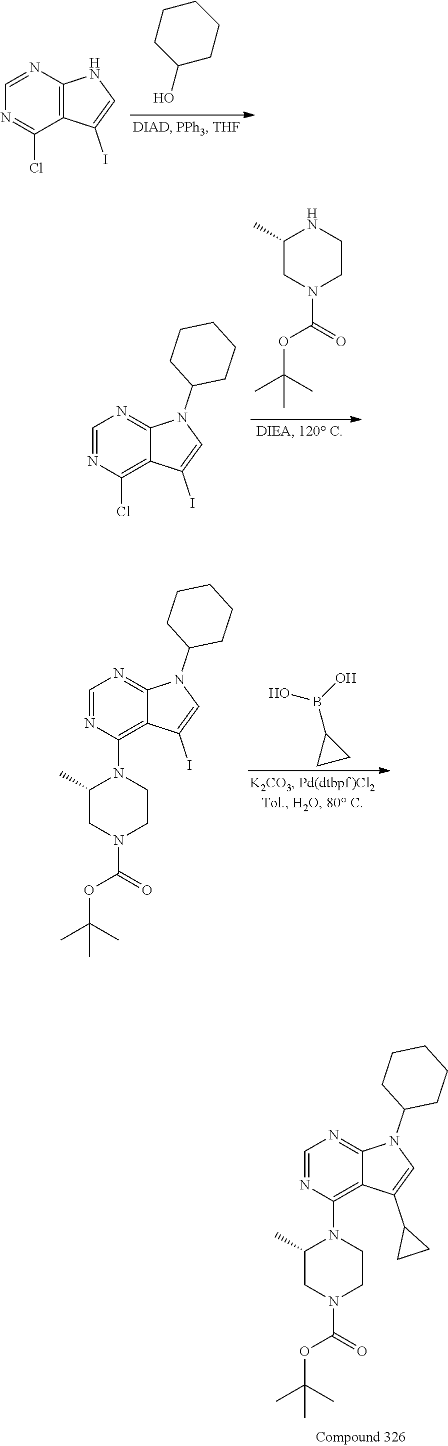

### Step 1. 4-Chloro-7-cyclohexyl-5-iodo-7H-pyrrolo[2,3-d]pyrimidine

To a solution of 4-chloro-5-iodo-7H-pyrrolo[2,3-d]pyrimidine (7.0 g, 25 mmol), cyclohexanol (10 g, 100 mmol) and PPh₃ (20 g, 75 mmol) in THE (40 mL) were added DIAD (15 g, 15 mL, 75 mmol) at 0° C. The resulting mixture was stirred at room temperature under N₂ atmosphere overnight. The solvent was removed under reduced pressure. The residue is dissolved with petroleum ether and filtered to remove solids. The solvent of filtrate was removed under reduced pressure. The residue was extract with EtOAc twice, the combined organic layers were dried over Na₂SO₄, filtered and concentrated. The residue was purified by flash chromatography (silica gel, 0˜30% EtOAc in petroleum ether) to afford 4-chloro-7-cyclohexyl-5-iodo-7H-pyrrolo[2,3-d]pyrimidine (2.2 g, 24% yield) as a white solid. LC/MS ESI (m/z): 362 (M+H)⁺.

### Step 2. tert-Butyl (S)-4-(7-cyclohexyl-5-iodo-7H-pyrrolo[2,3-d]pyrimidin-4-yl)-3-methylpiperazine-1-carboxylate

To the solution of 4-chloro-7-cyclohexyl-5-iodo-7H-pyrrolo[2,3-d]pyrimidine (1.8 g, 5.0 mmol) in DIEA (5 mL) was added tert-butyl (S)-3-methylpiperazine-1-carboxylate (5.0 g, 25 mmol). The resulting reaction mixture was stirred at 120° C. under N₂ overnight. After being cooled down to room temperature, the reaction mixture was concentrated under reduced pressure. The crude product was purified by flash column chromatography (silica gel, 0˜30%, ethyl acetate in petroleum ether) to afford tert-butyl (S)-4-(7-cyclohexyl-5-iodo-7H-pyrrolo[2,3-d]pyrimidin-4-yl)-3-methylpiperazine-1-carboxylate (1.6 g, 59% yield) as white solid. LC/MS ESI (m/z): 526 (M+H)⁺.

### Step 3. tert-Butyl (S)-4-(7-cyclohexyl-5-cyclopropyl-7H-pyrrolo[2,3-d]pyrimidin-4-yl)-3-methylpiperazine-1-carboxylate

To the solution of tert-butyl (S)-4-(7-cyclohexyl-5-iodo-7H-pyrrolo[2,3-d]pyrimidin-4-yl)-3-methylpiperazine-1-carboxylate (390 mg, 0.75 mmol) and cyclopropylboronic acid (260 mg, 3.0 mmol) in toluene (10 mL) were added Pd(dtbpf)Cl₂ (49 mg, 0.075 mmol), K₂CO₃ (2.1 g, 15 mmol) and H₂O (270 mg, 0.27 mL, 15 mmol) respectively. The resulting reaction mixture was stirred at 80° C. under N₂ overnight. After being cooled down to room temperature, the solvent was removed under reduced pressure. The residue was extracted with EtOAc twice. The combined organic layers were dried over Na₂SO₄, filtered, and concentrated under reduced pressure. The residue was purified by flash column chromatography (silica gel, 0˜40%, ethyl acetate in petroleum ether) to afford the crude product tert-butyl (S)-4-(7-cyclohexyl-5-cyclopropyl-7H-pyrrolo[2,3-d]pyrimidin-4-yl)-3-methylpiperazine-1-carboxylate (290 mg, 88% yield) as yellow solid. 50 mg of product was further purified by prep-HPLC (Gilson, C18, MeCN in H₂O) to afford pure product (16.9 mg). LC/MS ESI (m/z): 440 (M+H)⁺. ¹H NMR (400 MHz, MeOD) δ 8.23 (s, 1H), 7.01 (s, 1H), 4.80-4.68 (m, 1H), 4.57 (ddd, J=12.1, 7.9, 3.6 Hz, 1H), 4.05 (d, J=13.1 Hz, 1H), 3.84 (d, J=12.4 Hz, 2H), 3.57-3.45 (m, 1H), 3.45-3.33 (m, 1H), 3.24-3.03 (m, 1H), 2.07-1.98 (m, 1H), 1.98-1.83 (m, 4H), 1.82-1.66 (m, 3H), 1.61-1.50 (m, 2H), 1.49 (s, 9H), 1.40-1.29 (m, 1H), 1.16 (d, J=6.6 Hz, 3H), 1.03-0.91 (m, 2H), 0.85-0.73 (m, 1H), 0.69-0.56 (m, 1H).

The following compounds were prepared by the procedure similar to the synthesis of compound 326 from the corresponding alcohols.

| Cmpd | | |
|---|---|---|
| No. | Chemical Name | LCMS and ¹H NMR |
| 345 | tert-butyl (3S)-4-(5-cyclopropyl- | LC/MS ESI (m/z): 442 (M + H)⁺. ¹H NMR (400 MHz, |
| | 7-(tetrahydro-2H-pyran-3-yl)- | CDCl₃) δ 8.39 (s, 1H), 6.91 (s, 1H), 4.93-4.66 (m, 2H), |
| | 7H-pyrrolo[2,3-d]pyrimidin-4- | 4.26-3.78 (m, 6H), 3.68-3.48 (m, 2H), 3.40-3.21 (m, |
| | yl)-3-methylpiperazine-1- | 1H), 3.21-2.99 (m, 1H), 2.20-1.89 (m, 4H), 1.89-1.81 |
| | carboxylate | (m, 1H), 1.49 (s, 9H), 1.23 (s, 4H), 0.96 (d, J = 8.0 Hz, |
| | | 2H), 0.80-0.59 (m, 2H). |
| 364 | tert-butyl (S)-4-(7- | LC/MS ESI (m/z): 466 (M + H)⁺. ¹H NMR (400 MHz, |
| | (bicyclo[2.2.2]octan-1-yl)-5- | CDCl₃) δ 8.38 (s, 1H), 6.75 (s, 1H), 4.85-4.49 (m, 1H), |
| | cyclopropyl-7H-pyrrolo[2,3- | 4.12-3.90 (m, 1H), 3.88-3.63 (m, 2H), 3.60-3.45 (m, |
| | d]pyrimidin-4-yl)-3- | 1H), 3.44-3.26 (m, 1H), 3.26-3.03 (m, 1H), 2.42-2.19 |
| | methylpiperazine-1-carboxylate | (m, 6H), 2.03-1.95 (m, 1H), 1.89-1.75 (m, 6H), 1.72-1.69 |
| | | (m, 1H), 1.49 (s, 9H), 1.18 (d, J = 6.1 Hz, 3H), 1.00-0.85 |
| | | (m, 2H), 0.78-0.65 (m, 1H), 0.65-0.51 (m, 1H). |
| 361 | tert-butyl (S)-4-(7-((1S,2R,4R)- | LC/MS ESI (m/z): 452 (M + H)⁺. ¹H NMR (400 MHz, |
| | bicyclo[2.2.1]heptan-2-yl)-5- | CDCl₃) δ 8.39 (d, J = 1.9 Hz, 1H), 6.78 (s, 1H), |
| | cyclopropyl-7H-pyrrolo[2,3- | 5.21-5.04 (m, 1H), 4.76-4.68 (m, 1H), 4.12-3.77 (m, 3H), |
| | d]pyrimidin-4-yl)-3- | 3.54-3.06 (m, 3H), 2.79-2.59 (m, 1H), 2.44-2.38 (m, |
| | methylpiperazine-1-carboxylate | 1H), 2.24-2.04 (m, 1H), 2.02-1.95 (m, 1H), 1.77-1.67 |
| | | (m, 2H), 1.58-1.53 (m, 1H), 1.49 (s, 9H), 1.46-1.26 (m, |
| | | 4H), 1.21 (t, J = 6.6 Hz, 3H), 0.98-0.92 (m, 2H), |
| | | 0.76-0.59 (m, 2H). |

### Example 76. Synthesis of 1,1,1-trifluoro-2-methylpropan-2-yl (S)-4-(7-(3-cyanophenyl)-5-(1-methyl-1H-pyrazol-4-yl)-7H-pyrrolo[2,3-d]pyrimidin-4-yl)-3-methylpiperazine-1-carboxylate (Compound 329)

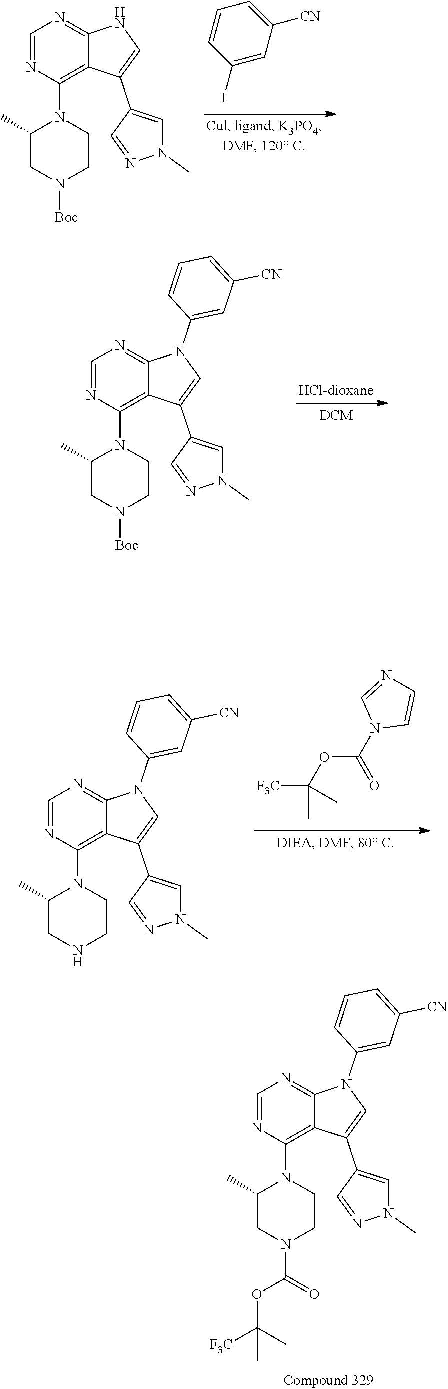

### Step 1. tert-Butyl (S)-4-(7-(3-cyanophenyl)-5-(I-methyl-1H-pyrazol-4-yl)-7H-pyrrolo[2,3-d]pyrimidin-4-yl)-3-methylpiperazine-1-carboxylate

A mixture of tert-butyl (S)-3-methyl-4-(5-(1-methyl-1H-pyrazol-4-yl)-7H-pyrrolo[2,3-d]pyrimidin-4-yl)piperazine-1-carboxylate (300 mg, 0.76 mmol, prepared following the procedure outlined in compound 321), 3-iodobenzonitrile (350 mg, 1.5 mmol), CuI (72 mg, 0.38 mmol), trans-1,2-diaminocyclohexane (26 mg, 0.23 mmol) and K₃PO₄ (480 mg, 2.3 mmol) in DMF (15 mL) was heated at 120° C. for 12 hours. The reaction mixture was concentrated. The residue was purified by column chromatography on silica gel (0˜70% EtOAc in petroleum ether, V/V) to give the tert-butyl (S)-4-(7-(3-cyanophenyl)-5-(1-methyl-1H-pyrazol-4-yl)-7H-pyrrolo[2,3-d]pyrimidin-4-yl)-3-methylpiperazine-1-carboxylate (300 mg, 80%) as a light yellow solid. LC/MS ESI (m/z): 499 (M+H)⁺.

### Step 2. (S)-3-(5-(1-Methyl-1H-pyrazol-4-yl)-4-(2-methylpiperazin-1-yl)-7H-pyrrolo[2,3-d]pyrimidin-7-yl)benzonitrile

To a mixture of tert-butyl (S)-4-(7-(3-cyanophenyl)-5-(1-methyl-1H-pyrazol-4-yl)-7H-pyrrolo[2,3-d]pyrimidin-4-yl)-3-methylpiperazine-1-carboxylate (260 mg, 0.52 mmol) in DCM (10 mL) was added HCl (5.0 mL, 4.0M in dioxane). The resulting mixture was stirred at room temperature for 3 hrs. The reaction mixture was concentrated to afford the (S)-3-(5-(1-methyl-1H-pyrazol-4-yl)-4-(2-methylpiperazin-1-yl)-7H-pyrrolo[2,3-d]pyrimidin-7-yl)benzonitrile (210 mg, 100%) as a yellow solid which was used in the next step directly without further purification. LC/MS ESI (m/z): 399 (M+H)⁺.

### Step 3. 1,1,1-Trifluoro-2-methylpropan-2-yl (S)-4-(7-(3-cyanophenyl)-5-(I-methyl-1H-pyrazol-4-yl)-7H-pyrrolo[2,3-d]pyrimidin-4-yl)-3-methylpiperazine-1-carboxylate

A mixture of (S)-3-(5-(1-methyl-1H-pyrazol-4-yl)-4-(2-methylpiperazin-1-yl)-7H-pyrrolo[2,3-d]pyrimidin-7-yl)benzonitrile (240 mg, 0.60 mmol), 1,1,1-trifluoro-2-methylpropan-2-yl 1H-imidazole-1-carboxylate (150 mg, 0.66 mmol) and DIEA (470 mg, 3.6 mmol) in DMF (10 mL) was heated at 80° C. for 12 hours. The reaction mixture was diluted with water, extracted with EtOAc (100 m1×2). The combined layers were washed with brine, dried over Na₂SO₄ and concentrated. The residue was purified by column chromatography on silica gel (0˜80% EtOAc in petroleum ether, V/V) to give the crude product. The crude product was purified by prep-HPLC to give the 1,1,1-trifluoro-2-methylpropan-2-yl (S)-4-(7-(3-cyanophenyl)-5-(1-methyl-1H-pyrazol-4-yl)-7H-pyrrolo[2,3-d]pyrimidin-4-yl)-3-methylpiperazine-1-carboxylate (27 mg, 8.0%) as a light yellow solid. LC/MS ESI (m/z): 553 (M+H)^{+ 1}H NMR δ 8.42 (d, J=3.2 Hz, 1H), 8.29 (s, 1H), 8.16-8.11 (m, 1H), 7.85 (s, 1H), 7.75-7.68 (m, 4H), 4.22 (br, 1H), 3.99 (s, 3H), 3.94-3.77 (m, 1H), 3.63-3.53 (m, 2H), 3.25 (d, J=12.6 Hz, 1H), 3.13-2.91 (m, 2H), 1.67 (s, 6H), 1.00 (d, J=6.7 Hz, 3H).

The following compound was prepared by the procedure analogous to the synthesis of compound 329 from the corresponding aryl halide.

| ID | Chemical Name | LCMS and ¹H NMR |
|---|---|---|
| 330 | 1,1,1-trifluoro-2-methylpropan- | LC/MS ESI (m/z): 554.2 (M + H)⁺. ¹H NMR (400 MHz, |
| | 2-yl (S)-4-(7-(4-cyanopyridin-2- | CD₃OD) δ 9.26 (d, J = 1.0 Hz, 1H), 8.68 (dd, J = 5.0, 0.8 |
| | yl)-5-(1-methyl-1H-pyrazol-4- | Hz, 1H), 8.54 (s, 1H), 8.22 (s, 1H), 7.87 (s, 1H), 7.71 (s, |
| | yl)-7H-pyrrolo[2,3-d]pyrimidin- | 1H), 7.58 (dd, J = 5.0, 1.3 Hz, 1H), 4.20 (br, 1H), 3.99 (s, |
| | 4-yl)-3-methylpiperazine-1- | 3H), 3.93-3.77 (m, 1H), 3.57 (br, 2H), 3.28-3.22 (m, |
| | carboxylate | 1H), 3.13-2.92 (m, 2H), 1.67 (s, 6H), 1.00 (d, J = 6.7 Hz, |
| | | 3H). |

### Example 77. Synthesis of tert-butyl (S)-4-(7-cyclohexyl-5-(pyrrolidin-1-yl)-7H-pyrrolo[2,3-d]pyrimidin-4-yl)-3-methylpiperazine-1-carboxylate (Compound 334)

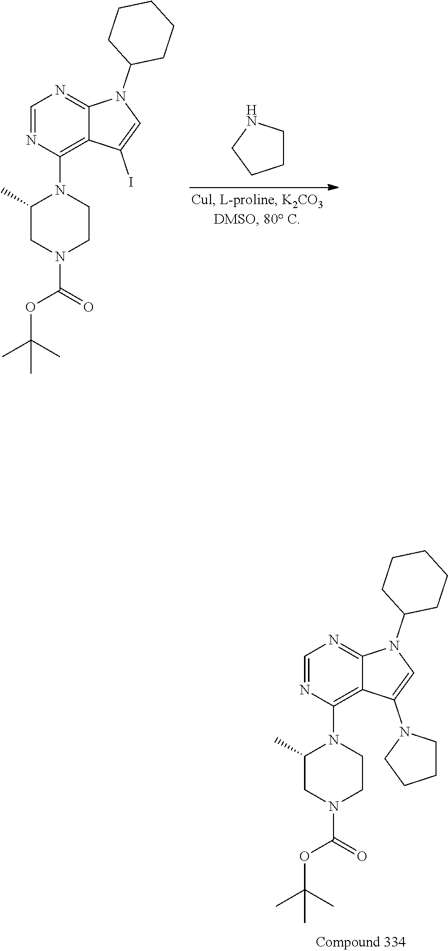 [SMILES: C[C@H]1CN(C(=O)OC(C)(C)C)CCN1c1ncnc2c1c(I)cn2C1CCCCC1 ; C1CCNC1 ; C[C@H]1CN(C(=O)OC(C)(C)C)CCN1c1ncnc2c1c(N1CCCC1)cn2C1CCCCC1]

### Step 1. tert-Butyl (S)-4-(7-cyclohexyl-5-(pyrrolidin-1-yl)-7H-pyrrolo[2,3-d]pyrimidin-4-yl)-3-methylpiperazine-1-carboxylate

To the solution of tert-butyl (S)-4-(7-cyclohexyl-5-iodo-7H-pyrrolo[2,3-d]pyrimidin-4-yl)-3-methylpiperazine-1-carboxylate (530 mg, 1.0 mmol, prepared following the procedure of compound 326, step 3) in DMSO (2 mL) were added CuI (38 mg, 0.20 mmol), L-proline (46 mg, 0.40 mmol), K₂CO₃ (410 mg, 3.0 mmol) and pyrrolidine (710 mg, 10 mmol) respectively. The reaction mixture was stirred at 80° C. under N₂ overnight. After being cooled down to room temperature, the mixture was extracted with EtOAc twice. The combined organic layers were dried over Na₂SO₄, filtered, and concentrated under reduced pressure. The residue was purified by flash column chromatography (silica gel, 0-40%, ethyl acetate in petroleum ether) to afford the crude product (110 mg) as yellow solid. 50 mg of product was further purified by prep-HPLC (Gilson, C18, MeCN in H₂O) to afford tert-butyl (S)-4-(7-cyclohexyl-5-(pyrrolidin-1-yl)-7H-pyrrolo[2,3-d]pyrimidin-4-yl)-3-methylpiperazine-1-carboxylate (6.3 mg, 1.3% yield). LC/MS ESI (m/z): 469 (M+H)⁺. ¹H NMR (400 MHz, MeOD) δ 8.15 (s, 1H), 6.82 (s, 1H), 5.07 (s, 1H), 4.59 (ddd, J=12.1, 7.9, 3.6 Hz, 1H), 4.25-3.98 (m, 2H), 3.90 (d, J=13.2 Hz, 1H), 3.39-3.32 (m, 1H), 3.30-3.20 (m, 1H), 3.19-3.09 (m, 2H), 3.09-2.90 (m, 1H), 2.90-2.76 (m, 2H), 2.07-1.85 (m, 8H), 1.84-1.69 (m, 3H), 1.60-1.51 (m, 2H), 1.48 (s, 9H), 1.40-1.30 (m, 1H), 1.05 (d, J=6.5 Hz, 3H).

The following compound was prepared by the procedure similar to the synthesis of compound 334 from the corresponding amine.

| ID | Chemical Name | LCMS and ¹H NMR |
|---|---|---|
| 344 | tert-butyl (S)-4-(7-cyclohexyl-5- | LC/MS ESI (m/z): 485 (M + H)⁺. ¹H NMR (400 MHz, |
| | morpholino-7H-pyrrolo[2,3- | CDCl₃) δ 8.33 (s, 1H), 6.60 (s, 1H), 5.17-4.90 (m, 1H), |
| | d]pyrimidin-4-yl)-3- | 4.77-4.62 (m, 1H), 4.39-3.98 (m, 2H), 3.97-3.74 (m, |
| | methylpiperazine-1-carboxylate | 5H), 3.49-3.36 (m, 1H), 3.34-3.19 (m, 1H), 3.16-2.71 |
| | | (m, 5H), 2.04 (d, J = 10.9 Hz, 2H), 1.90 (d, J = 12.7 Hz, |
| | | 2H), 1.77 (d, J = 13.1 Hz, 1H), 1.65-1.50 (m, 4H), 1.49 |
| | | (s, 9H), 1.29-1.20 (m, 1H), 1.18-1.03 (m, 3H). |

### Example 78. Synthesis of tert-butyl-(S)-4-(7-(3-chlorophenyl)-5-(pyrrolidin-1-ylmethyl)-7H-pyrrolo[2,3-d]pyrimidin-4-yl)-3-methylpiperazine-1-carboxylate (Compound 337)

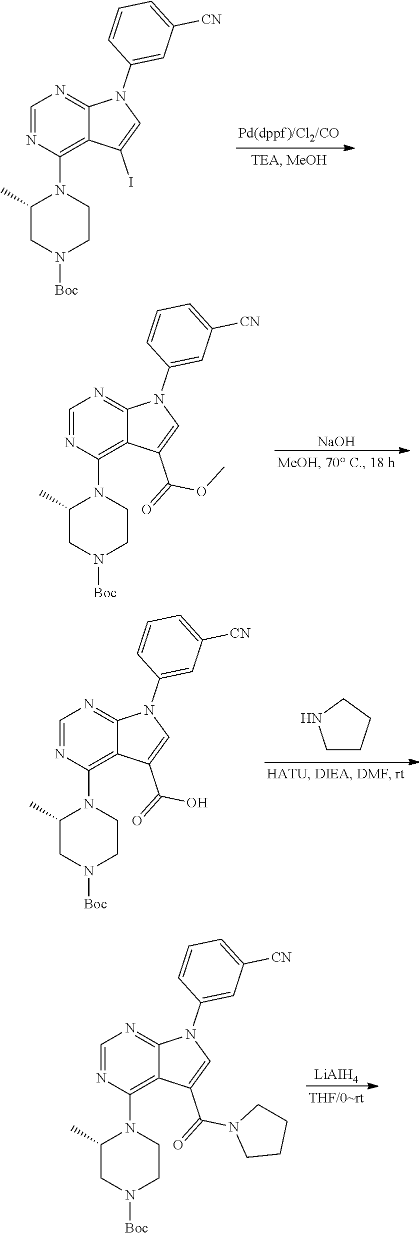

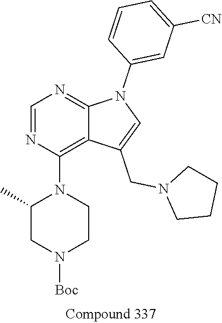 [SMILES: C[C@H]1CN(C(=O)OC(C)(C)C)CCN1c1ncnc2c1c(CN1CCCC1)cn2-c1cccc(C#N)c1]

### Step 1. Methyl-(S)-4-(4-(tert-butoxycarbonyl)-2-methylpiperazin-1-yl)-7-(3-chlorophenyl)-7H-pyrrolo[2,3-d]pyrimidine-5-carboxylate

A suspension of tert-butyl (3S)-4-[7-(3-chlorophenyl)-5-iodo-7H-pyrrolo[2,3-d]pyrimidin-4-yl]-3-methylpiperazine-1-carboxylate (500 mg, 0.90 mmol, prepared following the procedure outlined in compound 274), TEA (0.37 mL, 2.7 mmol) and Pd(dppf)Cl₂ (99 mg, 0.13 mmol) in MeOH (7 mL) was stirred at 70° C. under CO atmosphere for 18 h, After being cooled down to room temperature, solvent was removed. The residue was purified by column chromatography on silica gel (0-40% ethyl acetate in petroleum) to give tert-butyl (3S)-4-[7-(3-chlorophenyl)-5-(methoxycarbonyl)-7H-pyrrolo[2,3-d]pyrimidin-4-yl]-3-methylpiperazine-1-carboxylate (400 mg, 91%) as a white solid. LC/MS (ESI) (m/z): 486 (M+H)⁺

### Step 2. (S)-4-(4-(tert-Butoxycarbonyl)-2-methylpiperazin-1-yl)-7-(3-chlorophenyl)-7H-pyrrolo[2,3-d]pyrimidine-5-carboxylic acid

A solution of tert-butyl-(3S)-4-[7-(3-chlorophenyl)-5-(methoxycarbonyl)-7H-pyrrolo[2,3-d]pyrimidin-4-yl]-3-methylpiperazine-1-carboxylate (400 mg, 0.82 mmol) in MeOH (5 mL) was added 2.0M NaOH (1.0 mL), the resulting mixture was stirred at 80° C. for 18 h. After being cooled down to room temperature, the reaction was acidified with 1N HCl to PH 3, then was extracted by EtOAc twice. The combined organic phases were dried over anhydrous Na₂SO₄, filtered and concentrated. The residue was used in the next step directly. LC/MS(ESI) m/z: 472 (M+H)⁺

### Step 3. tert-Butyl-(S)-4-(7-(3-chlorophenyl)-5-(pyrrolidine-1-carbonyl)-7H-pyrrolo[2,3-d]pyrimidin-4-yl)-3-methylpiperazine-1-carboxylate

A suspension of 4-[(2S)-4-[(tert-butoxy)carbonyl]-2-methylpiperazin-1-yl]-7-(3-chlorophenyl)-7H-pyrrolo[2,3-d]pyrimidine-5-carboxylic acid (150 mg, 0.32 mmol), HATU (140 mg, 0.37 mmol) and DIEA (100 mg, 0.80 mmol) in DMF (5.0 mL) was stirred at room temperature for 0.5 h, then the mixture was added pyrrolidine (0.030 mL, 0.38 mmol) and stirred for another 2.5 h. The reaction mixture was partitioned between EtOAc and water, organic layer was separated, the aqueous layer was extracted with EtOAc twice. The combined organic phases were washed with brine, dried over anhydrous Na₂SO₄, filtered and concentrated. The residue was purified by column chromatography on silica gel (0˜15% MeOH in DCM) to get tert-butyl-(3S)-4-[7-(3-chlorophenyl)-5-(pyrrolidine-1-carbonyl)-7H-pyrrolo[2,3-d]pyrimidin-4-yl]-3-methylpiperazine-1-carboxylate (160 mg, 96%) as a white solid. LC/MS(ESI) m/z: 525 (M+H)⁺

### Step 4. tert-Butyl-(S)-4-(7-(3-chlorophenyl)-5-(pyrrolidin-1-ylmethyl)-7H-pyrrolo[2,3-d]pyrimidin-4-yl)-3-methylpiperazine-1-carboxylate

At 0° C., to a solution of tert-butyl-(3S)-4-[7-(3-chlorophenyl)-5-(pyrrolidine-1-carbonyl)-7H-pyrrolo[2,3-d]pyrimidin-4-yl]-3-methylpiperazine-1-carboxylate (80 mg, 0.15 mmol) in THF (3.0 mL) was added 1.0M LiAlH₄ (0.38 mL, THE solution). The resulting mixture was stirred for 3 h. The mixture was added 0.15 mL 10% NaOH. The resulting suspension was stirred for 1 h, filtered off and then washed with EtOAc twice, dried over anhydrous Na₂SO₄, filtered and concentrated. The residue was purified by prep-HPLC to give tert-butyl-(S)-4-(7-(3-chlorophenyl)-5-(pyrrolidin-1-ylmethyl)-7H-pyrrolo[2,3-d]pyrimidin-4-yl)-3-methylpiperazine-1-carboxylate (20 mg, 26%) as a white solid. LC/MS (ESI) (m/z): 511 (M+H)⁺. ¹H NMR (400 MHz, CDCl₃) δ 8.56 (s, 1H), 7.89 (s, 1H), 7.82 (t, J=1.9 Hz, 1H), 7.67-7.61 (m, 1H), 7.45 (t, J=8.1 Hz, 1H), 7.35 (d, J=9.0 Hz, 1H), 4.42 (d, J=13.2 Hz, 1H), 4.18 (dd, J=11.3, 5.0 Hz, 1H), 4.11 (d, J=13.4 Hz, 1H), 3.95-3.81 (m, 1H), 3.66 (d, J=13.4 Hz, 1H), 3.54-3.43 (m, 3H), 3.37-3.25 (m, 1H), 3.16-3.05 (m, 2H), 3.01-2.91 (m, 2H), 2.05-1.92 (m, 4H), 1.49 (s, 9H), 1.18 (d, J=6.4 Hz, 3H).

### Example 79. Synthesis of tert-butyl (S)-4-(7-cyclopentyl-5-(pyrrolidin-1-yl)-7H-pyrrolo[2,3-d]pyrimidin-4-yl)-3-methylpiperazine-1-carboxylate (Compound 338)

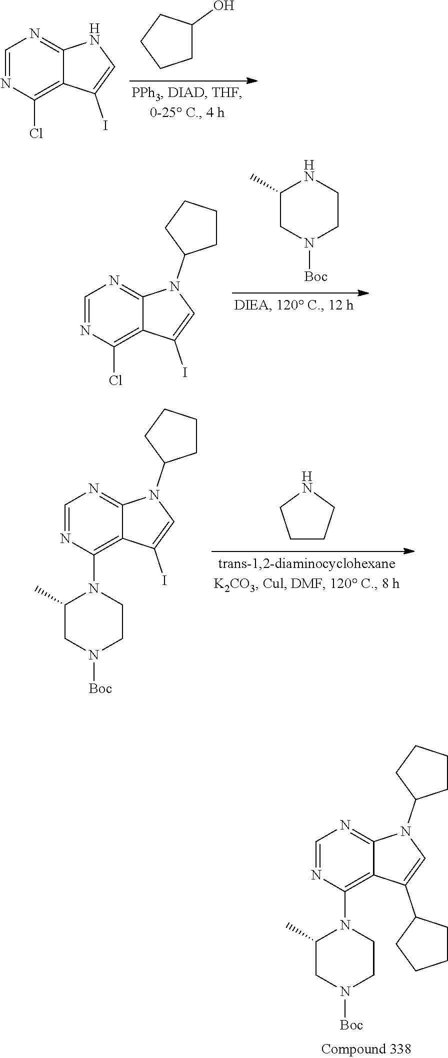

### Step 1. 4-Chloro-7-cyclopentyl-5-iodo-7H-pyrrolo[2,3-d]pyrimidine

At 0° C., to a solution of 4-chloro-5-iodo-7H-pyrrolo[2,3-d]pyrimidine (2.0 g, 7.2 mmol) in THF (10 mL) were added cyclopentanol (2.6 mL, 29 mmol), PPh₃ (5.6 g, 22 mmol) and DIAD (4.3 mL, 22 mmol) dropwise. The reaction mixture was stirred at 0° C. for 1 h and then was allowed to warm up to room temperature, and stirred for 3 h. TLC (petroleum ether:EtOAc=3:1) showed most 4-chloro-5-iodo-7H-pyrrolo[2,3-d]pyrimidine (R_{f}=0.30) was consumed and two spot (R_{f}=0.60, 0.70) were formed. The mixture was poured into H₂O (20 mL) and extracted with EtOAc (20 mL×4). The combined organic phases were washed with brine, dried over Na₂SO₄, filtered and concentrated under vacuo. The residue was purified by flash column chromatography to afford 4-chloro-7-cyclopentyl-5-iodo-7H-pyrrolo[2,3-d]pyrimidine (1.7 g, 4.9 mmol) was as an white solid. LC/MS ESI (m/z): 348 (M+H)⁺.

### Step 2. tert-Butyl (S)-4-(7-cyclopentyl-5-iodo-7H-pyrrolo[2,3-d]pyrimidin-4-yl)-3-methylpiperazine-1-carboxylate

To a solution of 4-chloro-7-cyclopentyl-5-iodo-7H-pyrrolo[2,3-d]pyrimidine (1.7 g, 4.9 mmol) in DIEA (4.9 mL, 29 mmol) was added tert-butyl (S)-3-methylpiperazine-1-carboxylate (2.0 g, 9.8 mmol) at 25° C. The mixture was degassed N₂ 3 times. The mixture was heated to 120° C. for 12 h. After being cooled down to room temperature, the reaction mixture was diluted with water, extracted with EtOAc (50 mL×4). The combined organic phase was washed with brine, dried over Na₂SO₄, filtered and concentrated under vacuo. The crude product was purified by silica gel column (SiO₂, petroleum ether:EtOAc=50:1 to 30:1) to afford tert-butyl (S)-4-(7-cyclopentyl-5-iodo-7H-pyrrolo[2,3-d]pyrimidin-4-yl)-3-methylpiperazine-1-carboxylate (1.8 g, 3.5 mmol). LC/MS ESI (m/z): 512 (M+H)⁺.

### Step 3. tert-Butyl (S)-4-(7-cyclopentyl-5-(pyrrolidin-1-yl)-7H-pyrrolo[2,3-d]pyrimidin-4-yl)-3-methylpiperazine-1-carboxylate

To a solution of tert-butyl (S)-4-(7-cyclopentyl-5-iodo-7H-pyrrolo[2,3-d]pyrimidin-4-yl)-3-methylpiperazine-1-carboxylate (200 mg, 0.39 mmol) in DMF (10 mL) were added pyrrolidine (640 mg, 1.5 mmol), CuI (37 mg, 0.20 mmol), trans-1,2-diaminocyclohexane (180 mg, 1.5 mmol), and K₂CO₃ (110 mg, 0.78 mmol) at 25° C. The mixture was heated to 120° C. for 8 h. The mixture was cooled to 25° C. and pour into H₂O (50 m1), extracted with EtOAc (20 mL×4). The combined organic phases were washed with brine, dried over Na₂SO₄, filtered and concentrated under vacuo. The crude product was purified by Prep-HPLC to afford tert-butyl (S)-4-(7-cyclopentyl-5-(pyrrolidin-1-yl)-7H-pyrrolo[2,3-d]pyrimidin-4-yl)-3-methylpiperazine-1-carboxylate (20 mg, 0.044 mmol) was obtained as a white solid. LC/MS ESI (m/z): 455 (M+H)⁺. ¹H NMR (400 MHz, CDCl₃) δ 8.33 (s, 1H), 6.51 (s, 1H), 5.26-5.15 (m, 1H), 4.98 (s, 1H), 4.14 (s, 2H), 3.91 (s, 1H), 3.37 (s, 1H), 3.25 (s, 1H), 3.09 (s, 3H), 2.84 (s, 2H), 2.23-2.14 (m, 2H), 1.97-1.72 (m, 10H), 1.49 (s, 9H), 1.11 (s, 3H).

### Example 80. Synthesis of tert-butyl (S)-4-(5-cyclopropyl-7-(tetrahydro-2H-pyran-4-yl)-7H-pyrrolo[2,3-d]pyrimidin-4-yl)-3-methylpiperazine-1-carboxylate (Compound 340)

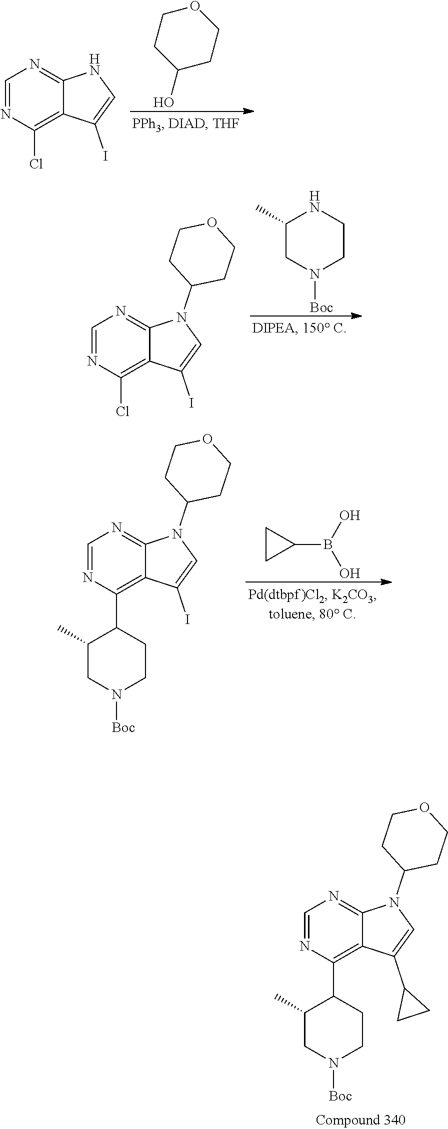

### Step 1. 4-Chloro-5-iodo-7-(tetrahydro-2H-pyran-4-yl)-7H-pyrrolo[2,3-d]pyrimidine

At 0° C., to a mixture of 4-chloro-5-iodo-7H-pyrrolo[2,3-d]pyrimidine (3.0 g, 11 mmol), tetrahydro-2H-pyran-4-ol (1.3 g, 13 mmol) and PPh₃ (5.6 g, 22 mmol) in THE (100 mL) was added DIAD (4.3 mL, 22 mmol) dropwise. The resulting mixture was stirred at room temperature for 12 hours. The reaction mixture was filtered. The filter cake was washed with EtOAc (50 m1) to give the 4-chloro-5-iodo-7-(tetrahydro-2H-pyran-4-yl)-7H-pyrrolo[2,3-d]pyrimidine (1.7 g, 44%) as a yellow solid which was used in the next step directly without further purification. LC/MS ESI (m/z): 364 (M+H)⁺.

### Step 2. tert-Butyl (S)-4-(5-iodo-7-(tetrahydro-2H-pyran-4-yl)-7H-pyrrolo[2,3-d]pyrimidin-4-yl)-3-methylpiperazine-1-carboxylate

A mixture of 4-chloro-5-iodo-7-(tetrahydro-2H-pyran-4-yl)-7H-pyrrolo[2,3-d]pyrimidine (1.7 g, 4.7 mmol) and tert-butyl (S)-3-methylpiperazine-1-carboxylate (1.9 g, 9.4 mmol) in DIEA (20 mL) was heated at 150° C. for 3 hours. The reaction mixture was concentrated. The residue was purified by column chromatography on silica gel (0˜70% EtOAc in petroleum ether, V/V) to give the tert-butyl (S)-4-(5-iodo-7-(tetrahydro-2H-pyran-4-yl)-7H-pyrrolo[2,3-d]pyrimidin-4-yl)-3-methylpiperazine-1-carboxylate (1.5 g, 59%) as a yellow solid. LC/MS ESI (m/z): 528 (M+H)⁺.

### Step 3. tert-Butyl (S)-4-(5-cyclopropyl-7-(tetrahydro-2H-pyran-4-yl)-7H-pyrrolo[2,3-d]pyrimidin-4-yl)-3-methylpiperazine-1-carboxylate

A mixture of tert-butyl (S)-4-(5-iodo-7-(tetrahydro-2H-pyran-4-yl)-7H-pyrrolo[2,3-d]pyrimidin-4-yl)-3-methylpiperazine-1-carboxylate (100 mg, 0.19 mmol), cyclopropylboronic acid (49 mg, 0.57 mmol), Pd(dtbpf)Cl₂ (25 mg, 0.040 mmol) and K₂CO₃ (520 mg, 3.8 mmol) in toluene (5 mL) was heated at 80° C. for 12 hours. The reaction mixture was concentrated. The residue was purified by column chromatography on silica gel (0˜80% EtOAc in petroleum ether, V/V) to give the crude product. The crude product was purified by prep-HPLC to give the tert-butyl (S)-4-(5-cyclopropyl-7-(tetrahydro-2H-pyran-4-yl)-7H-pyrrolo[2,3-d]pyrimidin-4-yl)-3-methylpiperazine-1-carboxylate (26 mg, 31%) as a white solid. LC/MS ESI (m/z): 442.3 (M+H)⁺. ¹H NMR (400 MHz, CD₃OD) δ 8.24 (s, 1H), 7.05 (s, 1H), 4.84-4.73 (m, 2H), 4.11-4.02 (m, 3H), 3.91-3.81 (m, 2H), 3.66-3.57 (m, 2H), 3.55-3.46 (m, 1H), 3.32 (br, 1H), 3.13 (br, 1H), 2.14-1.99 (m, 3H), 1.92-1.84 (m, 2H), 1.49 (s, 9H), 1.16 (d, J=6.6 Hz, 3H), 1.00-0.95 (m, 2H), 0.84-0.78 (m, 1H), 0.67-0.62 (m, 1H).

The following compound ware prepared by the procedure similar to the synthesis of compound 340 from the corresponding alcohol.

| Cmpd | | |
|---|---|---|
| No. | Chemical Name | LCMS and ¹H NMR |
| 339 | tert-butyl (S)-4-(7-cyclopentyl- | LC/MS ESI (m/z): 426.2 (M + H)⁺. ¹H NMR (400 MHz, |
| | 5-cyclopropyl-7H-pyrrolo[2,3- | CDCl3) δ 8.39 (s, 1H), 6.71 (s, 1H), 5.23-5.09 (m, 1H), |
| | d]pyrimidin-4-yl)-3- | 4.73 (s, 1H), 4.13-3.77 (m, 3H), 3.58-3.05 (m, 3H), |
| | methylpiperazine-1-carboxylate | 2.24-2.13 (m, 2H), 1.98 (s, 1H), 1.90-1.72 (m, 6H), 1.49 |
| | | (s, 9H), 1.21 (d, J = 6.0 Hz, 3H), 1.00-0.88 (m, 2H), |
| | | 0.75-0.58 (m, 2H). |

### Example 81. Synthesis of tert-butyl (S)-4-(3-cyclopropyl-1-(4-isocyanopyridin-2-yl)-1H-pyrazolo[3,4-d]pyrimidin-4-yl)-3-methylpiperazine-1-carboxylate (Compound 343)

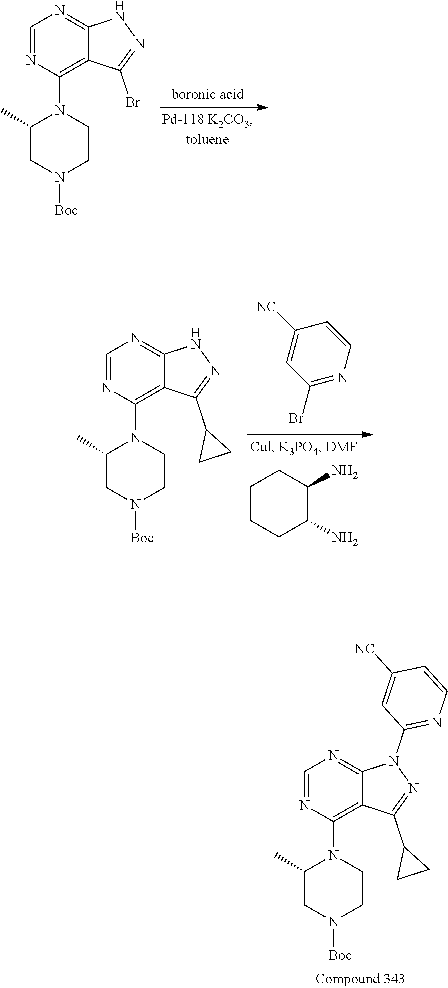

### Step 1. tert-Butyl (S)-4-(3-cyclopropyl-1H-pyrazolo[3,4-d]pyrimidin-4-yl)-3-methylpiperazine-1-carboxylate

The mixture of tert-butyl (S)-4-(3-bromo-1H-pyrazolo[3,4-d]pyrimidin-4-yl)-3-methylpiperazine-1-carboxylate (400 mg, 1.0 mmol), cyclopropylboronic acid (260 mg, 3.0 mmol), K₂CO₃ (4.1 g, 30 mmol), Pd-118 (97 mg, 0.15 mmol) and toluene (20 mL) was heated to 90° C. overnight. After being cooled down to room temperature, solvent was removed, the residue was purified by flash column chromatography (silica gel, 0˜15% MeOH in DCM) to afford tert-butyl (S)-4-(3-cyclopropyl-1H-pyrazolo[3,4-d]pyrimidin-4-yl)-3-methylpiperazine-1-carboxylate (110 mg, 30%). LC/MS ESI (m/z): 359 (M+H)⁺.

### Step 2. tert-Butyl (S)-4-(3-cyclopropyl-1-(4-isocyanopyridin-2-yl)-1H-pyrazolo[3,4-d]pyrimidin-4-yl)-3-methylpiperazine-1-carboxylate

To the solution of tert-butyl (3S)-4-{3-cyclopropyl-1H-pyrazolo[3,4-d]pyrimidin-4-yl}-3-methylpiperazine-1-carboxylate (100 mg, 0.28 mmol) in DMF (2 mL) were added CuI (27 mg, 0.14 mmol), K₃PO₄ (120 mg, 0.56 mmol), trans-cyclohexane-1,2-diamine (41 mg, 0.29 mmol) and 2-bromopyridine-4-carbonitrile (100 mg, 0.56 mmol) respectively. The resulting reaction mixture was stirred at 120° C. under N₂ overnight. After being cooled down to r.t., the reaction mixture was concentrated under reduced pressure. The residue was purified by flash column chromatography and prep-HPLC to afford tert-butyl (S)-4-(3-cyclopropyl-1-(4-isocyanopyridin-2-yl)-1H-pyrazolo[3,4-d]pyrimidin-4-yl)-3-methylpiperazine-1-carboxylate (30 mg, 23%) as solid. LC/MS ESI (m/z): 461.6 (M+H)⁺. ¹H NMR (400 MHz, CDCl₃) δ 8.88-8.80 (m, 1H), 8.60 (s, 1H), 8.53 (s, 1H), 7.46-7.39 (m, 1H), 5.11-4.95 (m, 1H), 4.43-4.34 (m, 1H), 4.30-3.92 (m, 2H), 3.67-3.50 (m, 1H), 3.30-2.94 (m, 2H), 2.10-2.02 (m, 1H), 1.51 (s, 9H), 1.38-1.33 (m, 5H), 1.20-1.13 (m, 2H).

### Example 82. Synthesis of ethyl (S)-4-(7-(4-cyanopyridin-2-yl)-5-(2-fluorophenyl)-7H-pyrrolo[2,3-d]pyrimidin-4-yl)-3-methylpiperazine-1-carboxylate (Compound 346)

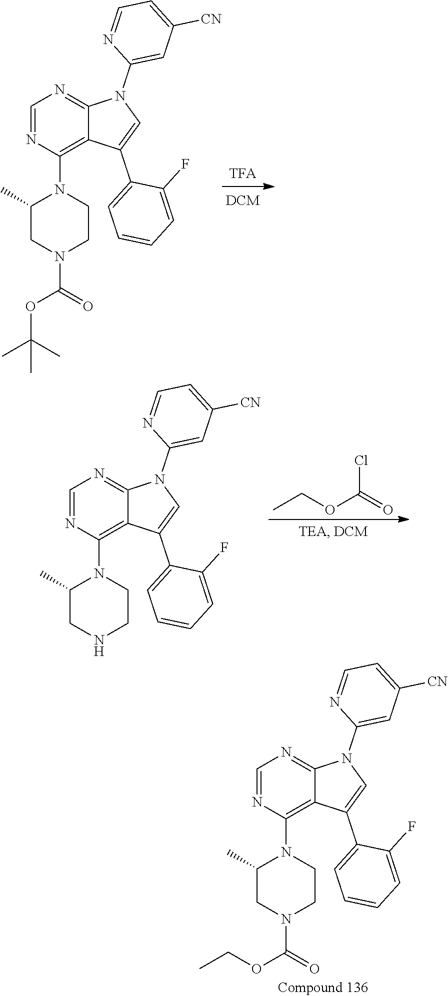

### Step 1. (S)-2-(5-(2-Fluorophenyl)-4-(2-methylpiperazin-1-yl)-7H-pyrrolo[2,3-d]pyrimidin-7-yl)isonicotinonitrile

At 0° C., to a solution of tert-butyl (S)-4-(7-(4-cyanopyridin-2-yl)-5-(2-fluorophenyl)-7H-pyrrolo[2,3-d]pyrimidin-4-yl)-3-methylpiperazine-1-carboxylate (500 mg, 0.16 mmol, prepared following the procedure outlined in compound 268) in DCM (10 mL) was added TFA (3 mL). The resulting mixture was stirred at the same temperature for 2 h. The reaction was quenched with NaHCO₃ (aq.), extracted with DCM twice, the combined organic layers were washed with brine, dried over Na₂SO₄, filtered and concentrated. The residue was used in the next step directly. LC/MS ESI (m/z): 414 (M+H)⁺.

### Step 2. Ethyl (S)-4-(7-(4-cyanopyridin-2-yl)-5-(2-fluorophenyl)-7H-pyrrolo[2,3-d]pyrimidin-4-yl)-3-methylpiperazine-1-carboxylate

At 0° C., to a solution of (S)-2-(5-(2-fluorophenyl)-4-(2-methylpiperazin-1-yl)-7H-pyrrolo[2,3-d]pyrimidin-7-yl)isonicotinonitrile (100 mg, 0.24 mmol) in DCM (10 mL) was added TEA (73 mg, 0.72 mmol), followed by ethyl carbonochloridate (52 mg, 0.48 mmol) dropwise. The resulting mixture was stirred at room temperature for 20 minutes. The reaction was quenched with NaHCO₃ (aq.), extracted with DCM twice, the combined organic layers were dried over Na₂SO₄, filtered and concentrated. The residue was purified by flash column chromatography (silica gel, 0˜30%, ethyl acetate in petroleum ether) to give crude product which was further purified by prep-HPLC to afford ethyl (S)-4-(7-(4-cyanopyridin-2-yl)-5-(2-fluorophenyl)-7H-pyrrolo[2,3-d]pyrimidin-4-yl)-3-methylpiperazine-1-carboxylate (70 mg, 59%) as a white solid. LC/MS ESI (m/z): 486 (M+H)⁺. ¹H NMR (400 MHz, CDCl3) δ 9.39 (s, 1H), 8.63 (d, J=5.0 Hz, 1H), 8.58 (s, 1H), 8.28 (s, 1H), 7.49-7.44 (m, 1H), 7.42-7.35 (m, 2H), 7.26-7.17 (m, 2H), 4.36-4.18 (m, 1H), 4.15-4.08 (m, 2H), 3.85-3.73 (m, 1H), 3.59 (d, J=12.9 Hz, 1H), 3.54-3.44 (m, 1H), 3.08 (d, J=11.8 Hz, 1H), 2.93-2.64 (m, 2H), 1.23 (t, J=7.1 Hz, 3H), 1.03 (s, 3H).

The following compounds were prepared by the procedure similar to the synthesis of compound 346 from the corresponding acid chlorides.

| Cmpd | | |
|---|---|---|
| No. | Chemical Name | LCMS and ¹H NMR |
| 347 | isopropyl (S)-4-(7-(4- | LC/MS ESI (m/z): 500 (M + H)⁺. ¹H NMR (400 MHz, |
| | cyanopyridin-2-yl)-5-(2- | CDCl3) δ 9.40 (s, 1H), 8.63-8.61 (m, 1H), 8.58 (s, 1H), |
| | fluorophenyl)-7H-pyrrolo[2,3- | 8.28 (s, 1H), 7.49-7.44 (m, 1H), 7.41-7.35 (m, 2H), |
| | d]pyrimidin-4-yl)-3- | 7.26-7.17 (m, 2H), 4.93-4.85 (m, 1H), 4.28 (s, 1H), |
| | methylpiperazine-1-carboxylate | 3.87-3.73 (m, 1H), 3.59 (d, J = 13.2 Hz, 1H), 3.49 (s, 1H), |
| | | 3.09 (t, J = 11.6 Hz, 1H), 2.83 (d, J = 44.2 Hz, 2H), |
| | | 1.23-1.19 (m, 6H), 1.02 (s, 3H). |
| 351 | methyl (S)-4-(7-(4-cyanopyridin- | LC/MS ESI (m/z): 472 (M + H)⁺. ¹H NMR (400 MHz, |
| | 2-yl)-5-(2-fluorophenyl)-7H- | CDCl3) δ 9.41 (s, 1H), 8.62 (d, J = 5.0 Hz, 1H), 8.58 (s, |
| | pyrrolo[2,3-d]pyrimidin-4-yl)-3- | 1H), 8.28 (s, 1H), 7.49-7.43 (m, 1H), 7.41-7.34 (m, 2H), |
| | methylpiperazine-1-carboxylate | 7.25-7.17 (m, 2H), 4.31-4.15 (m, 1H), 3.86-3.71 (m, |
| | | 1H), 3.69-3.66 (m, 3H), 3.61-3.44 (m, 2H), 3.09 (t, J = |
| | | 11.2 Hz, 1H), 2.91-2.65 (m, 2H), 1.03 (d, J = 5.8 Hz, 3H). |

### Example 83. Synthesis of 1,1,1-trifluoropropan-2-yl (3S)-4-(7-(4-cyanopyridin-2-yl)-5-(2-fluorophenyl)-7H-pyrrolo[2,3-d]pyrimidin-4-yl)-3-methylpiperazine-1-carboxylate (Compound 348)

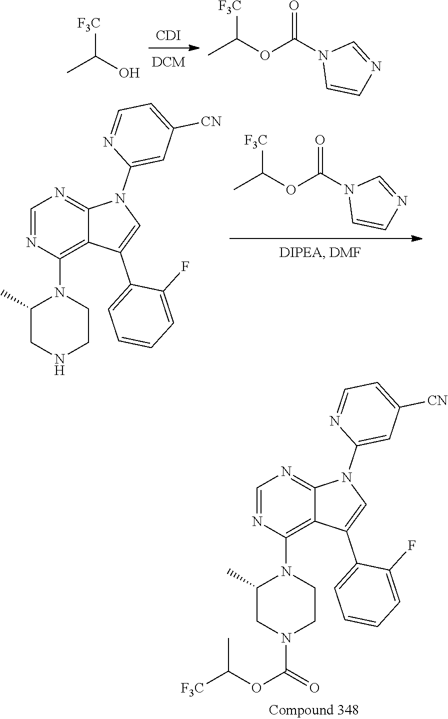

### Step 1. 1,1,1-Trifluoro-2-methylpropan-2-yl 1H-imidazole-1-carboxylate

To a solution of 1,1,1-trifluoropropan-2-ol (1.0 g, 8.8 mmol) in DCM (10 mL) was added di(1H-imidazol-1-yl)methanone (1.7 g, 11 mmol). The resulting mixture was stirred at room temperature overnight. The reaction was quenched with water, extracted with DCM twice, the combined organic layers were washed with brine, dried over Na₂SO₄, filtered and concentrated to afford 1,1,1-trifluoro-2-methylpropan-2-yl 1H-imidazole-1-carboxylate (1.1 g, 60%) as a white solid. LC/MS ESI (m/z): 209 (M+H)⁺.

### Step 2. 1,1,1-Trifluoropropan-2-yl (3S)-4-(7-(4-cyanopyridin-2-yl)-5-(2-fluorophenyl)-7H-pyrrolo[2,3-d]pyrimidin-4-yl)-3-methylpiperazine-1-carboxylate

To a solution of (S)-2-(5-(2-fluorophenyl)-4-(2-methylpiperazin-1-yl)-7H-pyrrolo[2,3-d]pyrimidin-7-yl)isonicotinonitrile (70 mg, 0.17 mmol, prepared following the procedure outlined in compound 346) in DMF (5 mL) were added 1,1,1-trifluoropropan-2-yl 1H-imidazole-1-carboxylate (71 mg, 0.34 mmol) and DIPEA (70 mg, 0.51 mmol). The resulting mixture was stirred at 80° C. for 2 days under N₂. The reaction was quenched with water, extracted with EtOAc twice, the combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford 1,1,1-trifluoropropan-2-yl (3S)-4-(7-(4-cyanopyridin-2-yl)-5-(2-fluorophenyl)-7H-pyrrolo[2,3-d]pyrimidin-4-yl)-3-methylpiperazine-1-carboxylate (29 mg, 30%) as a white solid. LC/MS ESI (m/z): 554 (M+H)⁺. ¹H NMR (400 MHz, CDCl3) δ 9.40 (s, 1H), 8.62 (d, J=5.0 Hz, 1H), 8.58 (s, 1H), 8.29 (s, 1H), 7.46 (t, J=7.5 Hz, 1H), 7.42-7.36 (m, 2H), 7.27-7.19 (m, 2H), 5.28-5.19 (m, 1H), 4.22 (d, J=28.8 Hz, 1H), 3.87-3.72 (m, 1H), 3.60-3.48 (m, 2H), 3.15-3.08 (m, 1H), 2.95-2.72 (m, 2H), 1.37 (d, J=6.5 Hz, 3H), 1.01 (t, J=6.1 Hz, 3H)

The following compound was prepared by the procedures similar to the synthesis of compound 348 from 2,2,2-trifluoroethan-1-ol.

| Cmpd | | |
|---|---|---|
| No. | Chemical Name | LCMS and ¹H NMR |
| 349 | 2,2,2-trifluoroethyl (S)-4-(7-(4- | LC/MS ESI (m/z): 540 (M + H)⁺. ¹H NMR (400 MHz, |
| | cyanopyridin-2-yl)-5-(2- | CDCl3) δ 9.41 (s, 1H), 8.63 (d, J = 5.0 Hz, 1H), 8.59 (s, |
| | fluorophenyl)-7H-pyrrolo[2,3- | 1H), 8.29 (s, 1H), 7.47 (t, J = 7.4 Hz, 1H), 7.42-7.36 (m, |
| | d]pyrimidin-4-yl)-3- | 2H), 7.27-7.18 (m, 2H), 4.59-4.35 (m, 2H), 4.23 (d, J = |
| | methylpiperazine-1-carboxylate | 19.1 Hz, 1H), 3.87-3.73 (m, 1H), 3.61-3.49 (m, 2H), |
| | | 3.12 (t, J = 11.4 Hz, 1H), 2.98-2.74 (m, 2H), 1.03 (t, |
| | | J = 6.3 Hz, 3H). |

### Example 84. Synthesis of tert-butyl (S)-4-(7-(4-cyanopyridin-2-yl)-5-(pyridin-2-yl)-7H-pyrrolo[2,3-d]pyrimidin-4-yl)-3-methylpiperazine-1-carboxylate (Compound 352)

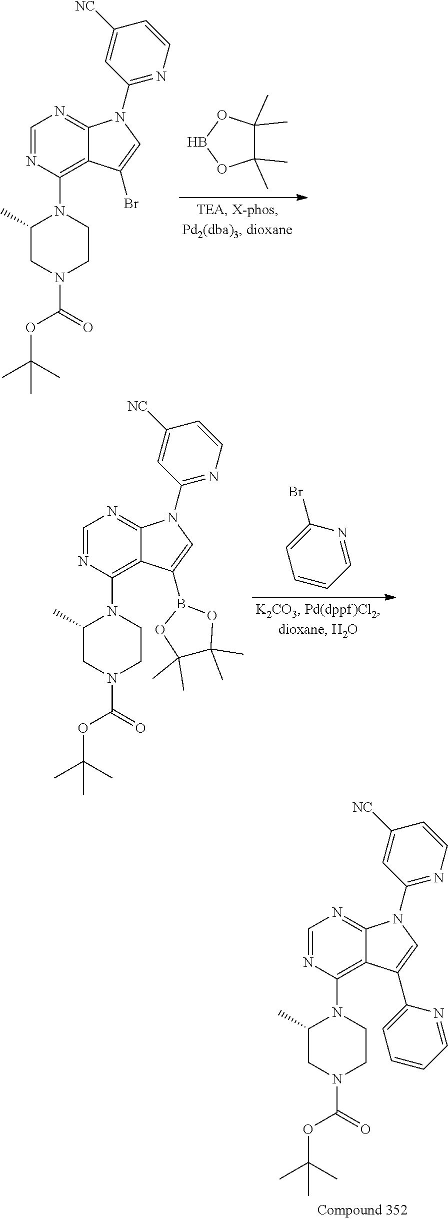

### Step 1. tert-Butyl (S)-4-(7-(4-cyanopyridin-2-yl)-5-(4,4,5,5-tetramethyl-1,3,2-dioxaborolan-2-yl)-7H-pyrrolo[2,3-d]pyrimidin-4-yl)-3-methylpiperazine-1-carboxylate

To a solution of tert-butyl (S)-4-(5-bromo-7-(4-cyanopyridin-2-yl)-7H-pyrrolo[2,3-d]pyrimidin-4-yl)-3-methylpiperazine-1-carboxylate (800 mg, 1.6 mmol, prepared following a similar procedure outlined in compound 388) in dioxane (15 mL) were added 4,4,5,5-tetramethyl-1,3,2-dioxaborolane (0.93 mL, 6.4 mmol), X-Phos (77 mg, 0.16 mmol), TEA (1.1 mL, 8.0 mmol) and Pd₂(dba)₃ (150 mg, 0.16 mmol). The resulting mixture was stirred at 95° C. for 5 h under N₂. After being cooled down to room temperature, the reaction was quenched with water, extracted with DCM twice, the combined organic layers were washed with brine, dried over Na₂SO₄, filtered and concentrated to afford crude tert-butyl (S)-4-(7-(4-cyanopyridin-2-yl)-5-(4,4,5,5-tetramethyl-1,3,2-dioxaborolan-2-yl)-7H-pyrrolo[2,3-d]pyrimidin-4-yl)-3-methylpiperazine-1-carboxylate (800 mg, 91%) as a black oil. LC/MS ESI (m/z): 546 (M+H)⁺.

### Step 2. tert-Butyl (S)-4-(7-(4-cyanopyridin-2-yl)-5-(pyridin-2-yl)-7H-pyrrolo[2,3-d]pyrimidin-4-yl)-3-methylpiperazine-1-carboxylate

To a solution of tert-butyl (S)-4-(7-(4-cyanopyridin-2-yl)-5-(4,4,5,5-tetramethyl-1,3,2-dioxaborolan-2-yl)-7H-pyrrolo[2,3-d]pyrimidin-4-yl)-3-methylpiperazine-1-carboxylate (200 mg, 0.36 mmol) in dioxane (10 mL) and H₂O (2 mL) were added 2-bromopyridine (0.070 mL, 0.73 mmol), K₂CO₃ (250 mg, 1.8 mmol) and Pd(dppf)Cl₂ (27 mg, 0.037 mmol). The resulting mixture was heated to 90° C. overnight. After being cooled down to room temperature, solvent was removed and the residue was purified by flash column chromatography (silica gel, 0˜90%, ethyl acetate in petroleum ether) to give crude product which was further purified by prep-HPLC to afford tert-butyl (S)-4-(7-(4-cyanopyridin-2-yl)-5-(pyridin-2-yl)-7H-pyrrolo[2,3-d]pyrimidin-4-yl)-3-methylpiperazine-1-carboxylate (26 mg, 14%) as a white solid. LC/MS ESI (m/z): 497 (M+H)⁺. ¹H NMR (400 MHz, CDCl₃) δ 9.39 (s, 1H), 8.73 (d, J=4.7 Hz, 1H), 8.66-8.55 (m, 3H), 7.84 (s, 1H), 7.64 (d, J=6.2 Hz, 1H), 7.42 (dd, J=5.0, 1.1 Hz, 1H), 7.32 (s, 1H), 4.30 (d, J=44.9 Hz, 1H), 3.92-3.71 (m, 1H), 3.64-3.43 (m, 2H), 3.14 (t, J=11.3 Hz, 1H), 3.03-2.60 (m, 2H), 1.43 (s, 9H), 1.05 (s, 3H).

The following compounds were prepared by the procedures similar to the synthesis of compound 352 from the corresponding aryl halides.

| Cmpd | | |
|---|---|---|
| No. | Chemical Name | LCMS and ¹H NMR |
| 354 | tert-butyl (S)-4-(7-(4- | LC/MS ESI (m/z): 497 (M + H)⁺. ¹H NMR (400 MHz, |
| | cyanopyridin-2-yl)-5-(pyridin-4- | CDCl₃) δ 9.42 (s, 1H), 8.66 (dd, J = 25.5, 20.6 Hz, 4H), |
| | yl)-7H-pyrrolo[2,3-d]pyrimidin- | 8.37 (s, 1H), 7.54 (s, 2H), 7.44 (d, J = 4.9 Hz, 1H), |
| | 4-yl)-3-methylpiperazine-1- | 4.36-4.09 (m, 1H), 4.00-3.43 (m, 3H), 3.25-3.07 (m, 1H), |
| | carboxylate | 3.04-2.87 (m, 1H), 2.84-2.67 (m, 1H), 1.43 (s, 9H), 1.05 |
| | | (s, 3H). |
| 355 | tert-butyl (S)-4-(7-(4- | LC/MS ESI (m/z): 515 (M + H)⁺. ¹H NMR (400 MHz, |
| | cyanopyridin-2-yl)-5-(3- | CDCl₃) δ 9.40 (s, 1H), 8.66 (dd, J = 5.0, 0.5 Hz, 1H), 8.62 |
| | fluoropyridin-4-yl)-7H- | (d, J = 2.4 Hz, 2H), 8.52 (d, J = 4.9 Hz, 1H), 8.50 (s, 1H), |
| | pyrrolo[2,3-d]pyrimidin-4-yl)-3- | 7.53-7.49 (m, 1H), 7.46 (dd, J = 5.0, 1.2 Hz, 1H), 4.26 (s, |
| | methylpiperazine-1-carboxylate | 1H), 3.85-3.54 (m, 2H), 3.42 (s, 1H), 3.14 (t, J = 11.8 Hz, |
| | | 1H), 2.93 (d, J = 11.2 Hz, 1H), 2.76 (s, 1H), 1.43 (s, 9H), |
| | | 1.08 (d, J = 6.6 Hz, 3H). |
| 180 | tert-butyl (2R,5S)-4-(7-(3,4- | LC/MS ESI (m/z): 536 (M + H)⁺. ¹H NMR (400 MHz, |
| | difluorophenyl)-5-(3- | CD₃OD) δ 8.61-8.56 (m, 2H), 8.44 (s, 1H), 7.90 (ddd, J = |
| | methylpyrazin-2-yl)-7H- | 11.5, 7.1, 2.6 Hz, 1H), 7.83 (s, 1H), 7.66-7.59 (m, 1H), |
| | pyrrolo[2,3-d]pyrimidin-4-yl)- | 7.53-7.43 (m, 1H), 4.21-4.14 (m, 1H), 4.13-3.95 (m, |
| | 2,5-dimethylpiperazine-1- | 1H), 3.39-3.32 (m, 2H), 3.16-3.08 (m, 1H), 2.84-2.66 |
| | carboxylate | (m, 1H), 2.55 (s, 3H), 1.43 (s, 9H), 0.98 (t, J = 6.3 Hz, 6H). |
| 181 | tert-butyl (2R,5S)-4-(7-(4- | LC/MS ESI (m/z): 518 (M + H)⁺. ¹H NMR (400 MHz, |
| | fluorophenyl)-5-(3- | MeOD) δ 8.58 (dd, J = 7.4, 2.6 Hz, 2H), 8.40 (s, 1H), |
| | methylpyrazin-2-yl)-7H- | 7.81-7.76 (m, 3H), 7.35-7.29 (m, 2H), 4.21-4.14 (m, 1H), |
| | pyrrolo[2,3-d]pyrimidin-4-yl)- | 4.11-3.95 (m, 1H), 3.38-3.31 (m, 2H), 3.16-3.09 (m, |
| | 2,5-dimethylpiperazine-1- | 1H), 2.84-2.66 (m, 1H), 2.55 (s, 3H), 1.43 (s, 9H), |
| | carboxylate | 0.99 (t, J = 0.8 Hz, 6H). |
| 182 | tert-butyl (2R,5S)-2,5-dimethyl- | LC/MS ESI (m/z): 500 (M + H)⁺. ¹H NMR (400 MHz, |
| | 4-(5-(3-methylpyrazin-2-yl)-7- | MeOD) δ 8.58 (dd, J = 7.5, 2.6 Hz, 2H), 8.41 (s, 1H), |
| | phenyl-7H-pyrrolo[2,3- | 7.80-7.75 (m, 3H), 7.61-7.56 (m, 2H), 7.46 (t, J = 7.5 Hz, |
| | d]pyrimidin-4-yl)piperazine-1- | 1H), 4.21-4.15 (m, 1H), 4.12-3.94 (m, 1H), 3.38-3.32 |
| | carboxylate | (m, 2H), 3.17-3.10 (m, 1H), 2.81-2.65 (m, 1H), 2.56 (s, |
| | | 3H), 1.43 (s, 9H), 0.99 (dd, J = 9.6, 6.7 Hz, 6H). |
| 184 | tert-butyl (2R,5S)-4-(7-(4- | LC/MS ESI (m/z): 534 (M + H)⁺. ¹H NMR (400 MHz, |
| | chlorophenyl)-5-(3- | MeOD) δ 8.58 (dd, J = 7.5, 2.6 Hz, 2H), 8.42 (s, 1H), |
| | methylpyrazin-2-yl)-7H- | 7.83-7.79 (m, 3H), 7.60-7.56 (m, 2H), 4.21-4.14 (m, 1H), |
| | pyrrolo[2,3-d]pyrimidin-4-yl)- | 4.12-3.93 (m, 1H), 3.39-3.31 (m, 2H), 3.16-3.08 (m, |
| | 2,5-dimethylpiperazine-1- | 1H), 2.84-2.67 (m, 1H), 2.55 (s, 3H), 1.43 (s, 9H), |
| | carboxylate | 0.98 (t, J = 7.0 Hz, 6H). |

### Example 85. Synthesis of tert-butyl (S)-4-(7-(4-cyanopyridin-2-yl)-5-(pyridin-3-yl)-7H-pyrrolo[2,3-d]pyrimidin-4-yl)-3-methylpiperazine-1-carboxylate (Compound 353)

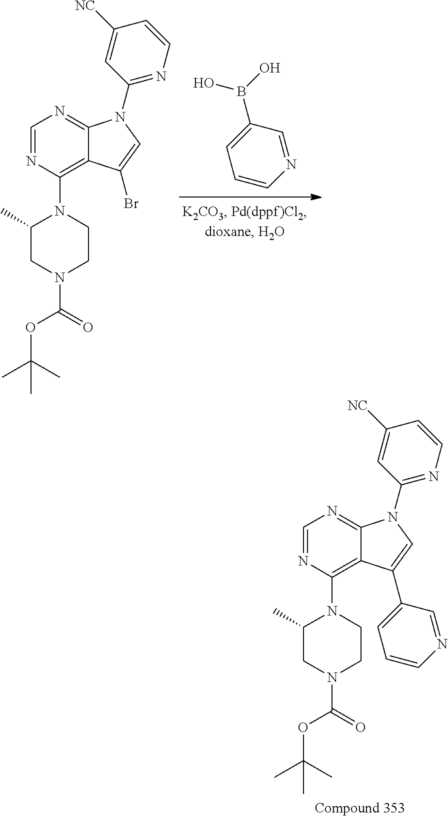 [SMILES: C[C@H]1CN(C(=O)OC(C)(C)C)CCN1c1ncnc2c1c(Br)cn2-c1cc(C#N)ccn1 ; OB(O)c1cccnc1 ; C[C@H]1CN(C(=O)OC(C)(C)C)CCN1c1ncnc2c1c(-c1cccnc1)cn2-c1cc(C#N)ccn1]

### Step 1. tert-Butyl (S)-4-(7-(4-cyanopyridin-2-yl)-5-(pyridin-3-yl)-7H-pyrrolo[2,3-d]pyrimidin-4-yl)-3-methylpiperazine-1-carboxylate

To a solution of tert-butyl (S)-4-(5-bromo-7-(4-cyanopyridin-2-yl)-7H-pyrrolo[2,3-d]pyrimidin-4-yl)-3-methylpiperazine-1-carboxylate (150 mg, 0.30 mmol, prepared following the procedure outlined in compound 388) in dioxane (10 mL) and H₂O (2 mL) was added pyridin-3-ylboronic acid (74 mg, 0.60 mmol), K₂CO₃ (210 mg, 1.5 mmol) and Pd(dppf)Cl₂ (22 mg, 0.030 mmol). The resulting mixture was heated to 90° C. overnight. After being cooled down to room temperature, solvent was removed and the residue was purified by flash column chromatography (silica gel, 0-90%, ethyl acetate in petroleum ether) to give crude product which was further purified by prep-HPLC to afford tert-butyl (S)-4-(7-(4-cyanopyridin-2-yl)-5-(pyridin-3-yl)-7H-pyrrolo[2,3-d]pyrimidin-4-yl)-3-methylpiperazine-1-carboxylate (84 mg, 56%) as a white solid. LC/MS ESI (m/z): 497 (M+H)⁺. ¹H NMR (400 MHz, CDCl₃) δ 9.42 (s, 1H), 8.88 (s, 1H), 8.67-8.60 (m, 3H), 8.33 (s, 1H), 7.98 (d, J=7.3 Hz, 1H), 7.52-7.45 (m, 1H), 7.44 (dd, J=5.0, 1.2 Hz, 1H), 4.12 (s, 1H), 3.91-3.69 (m, 1H), 3.61-3.51 (m, 1H), 3.43 (d, J=13.0 Hz, 1H), 3.14 (td, J=13.4, 2.9 Hz, 1H), 2.99-2.66 (m, 2H), 1.43 (s, 9H), 1.03 (d, J=5.4 Hz, 3H).

### Example 86. Synthesis of tert-butyl (S)-3-methyl-4-(5-(pyrrolidin-1-yl)-7-(3,4,5-trifluorophenyl)-7H-pyrrolo[2,3-d]pyrimidin-4-yl)piperazine-1-carboxylate (Compound 356)

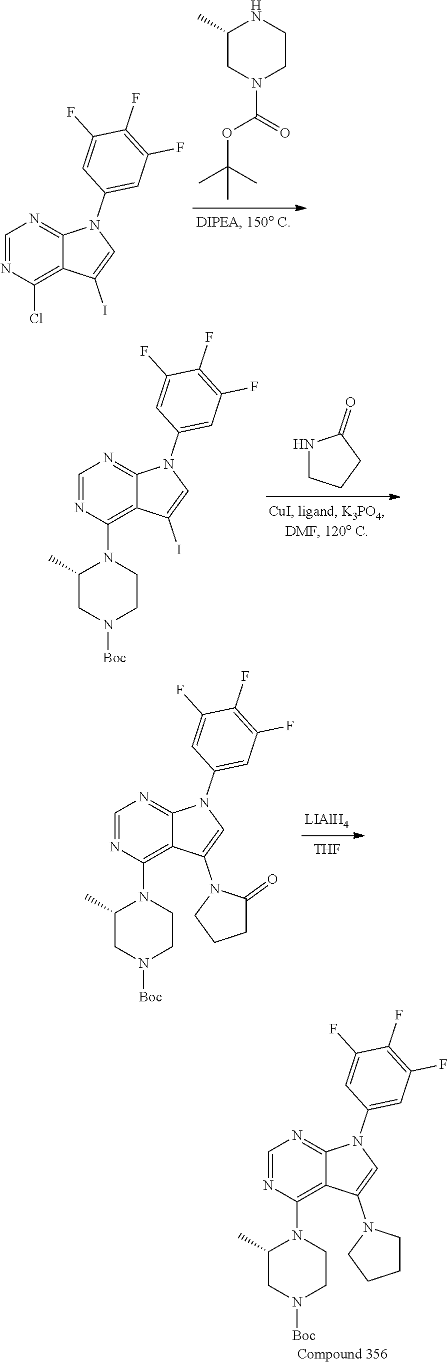

### Step 1. tert-Butyl (S)-4-(5-iodo-7-(3,4,5-trifluorophenyl)-7H-pyrrolo[2,3-d]pyrimidin-4-yl)-3-methylpiperazine-1-carboxylate

A mixture of 4-chloro-5-iodo-7-(3,4,5-trifluorophenyl)-7H-pyrrolo[2,3-d]pyrimidine (450 mg, 1.1 mmol, prepared following the procedure outline in compound 313) and tert-butyl (S)-3-methylpiperazine-1-carboxylate (440 mg, 2.2 mmol) in DIEA (5 mL) was heated at 150° C. for 3 hours. The reaction mixture was concentrated. The residue was purified by column chromatography on silica gel (0˜70% EtOAc in petroleum ether, V/V) to give the tert-butyl (S)-4-(5-iodo-7-(3,4,5-trifluorophenyl)-7H-pyrrolo[2,3-d]pyrimidin-4-yl)-3-methylpiperazine-1-carboxylate (530 mg, 84%) as a yellow solid. LC/MS ESI (m/z): 574 (M+H)⁺.

### Step 2. tert-Butyl (S)-3-methyl-4-(5-(2-oxopyrrolidin-1-yl)-7-(3,4,5-trifluorophenyl)-7H-pyrrolo[2,3-d]pyrimidin-4-yl)piperazine-1-carboxylate

A mixture of tert-butyl (S)-4-(5-iodo-7-(3,4,5-trifluorophenyl)-7H-pyrrolo[2,3-d]pyrimidin-4-yl)-3-methylpiperazine-1-carboxylate (530 mg, 0.92 mmol), pyrrolidin-2-one (240 mg, 2.8 mmol), CuI (88 mg, 0.46 mmol), trans-1,2-diaminocyclohexane (32 mg, 0.28 mmol) and K₃PO₄ (590 mg, 2.8 mmol) in DMF (10 mL) was heated at 120° C. for 12 hours. The reaction mixture was concentrated. The residue was purified by column chromatography on silica gel (0˜100% EtOAc in petroleum ether, V/V) to give the tert-butyl (S)-3-methyl-4-(5-(2-oxopyrrolidin-1-yl)-7-(3,4,5-trifluorophenyl)-7H-pyrrolo[2,3-d]pyrimidin-4-yl)piperazine-1-carboxylate (300 mg, 61%) as a grey oil. LC/MS ESI (m/z): 531 (M+H)⁺.

### Step 3. tert-Butyl (S)-3-methyl-4-(5-(pyrrolidin-1-yl)-7-(3,4,5-trifluorophenyl)-7H-pyrrolo[2,3-d]pyrimidin-4-yl)piperazine-1-carboxylate

At 0° C., to a solution of tert-butyl (S)-3-methyl-4-(5-(2-oxopyrrolidin-1-yl)-7-(3,4,5-trifluorophenyl)-7H-pyrrolo[2,3-d]pyrimidin-4-yl)piperazine-1-carboxylate (100 mg, 0.19 mmol) in THE (10 mL) was added LiAlH₄ (0.57 mL, 1.0 M in THF) dropwise. The resulting mixture was stirred at 0° C. for 30 mins. The reaction mixture was quenched with ice water, the resulting mixture was extracted with EtOAc (50 m1×2). The combined layers were washed with brine, dried over Na₂SO₄ and concentrated. The residue was purified by column chromatography on silica gel (0˜10% MeOH in DCM, V/V) to give the crude product. The crude product was purified by prep-HPLC to give the tert-butyl (S)-3-methyl-4-(5-(pyrrolidin-1-yl)-7-(3,4,5-trifluorophenyl)-7H-pyrrolo[2,3-d]pyrimidin-4-yl)piperazine-1-carboxylate (3.0 mg, 3.1%) as a white solid. LC/MS ESI (m/z): 517 (M+H)⁺. ¹H NMR (400 MHz, CDCl3) δ 8.37 (s, 1H), 7.51 (dd, J=8.8, 6.2 Hz, 2H), 6.63 (s, 1H), 4.97 (br, 1H), 4.23-3.87 (m, 3H), 3.38 (t, J=12.0 Hz, 1H), 3.30-2.98 (m, 4H), 2.91 (br, 2H), 1.98 (br, 4H), 1.49 (s, 9H), 1.13 (s, 3H).

The following compounds were prepared by the procedure similar to the synthesis of compound 356 from the corresponding aryl halides prepared following the procedure outlined in compound 340, step 1.

| Cmpd | | |
|---|---|---|
| No. | Chemical Name | LCMS and ¹H NMR |
| 360 | tert-butyl (S)-3-methyl-4-(5- | LC/MS ESI (m/z): 471.2 (M + H)⁺. ¹H NMR (400 MHz, |
| | (pyrrolidin-1-yl)-7-(tetrahydro- | CD₃OD) δ 8.17 (s, 1H), 6.86 (s, 1H), 5.07 (s, 1H), |
| | 2H-pyran-4-yl)-7H-pyrrolo[2,3- | 4.91-4.81 (m, 1H), 4.18-4.05 (m, 4H), 3.90 (d, J = 13.2 Hz, |
| | d]pyrimidin-4-yl)piperazine-1- | 1H), 3.62 (t, J = 11.9 Hz, 2H), 3.36 (dd, J = 12.8, 3.3 Hz, |
| | carboxylate | 1H), 3.25 (br, 1H), 3.14 (br, 2H), 3.02 (br, 1H), 2.90-2.79 |
| | | (m, 2H), 2.17-2.03 (m, 2H), 1.96 (br, 4H), 1.92-1.86 (m, |
| | | 2H), 1.48 (s, 9H), 1.05 (d, J = 6.6 Hz, 3H) . . . |

### Example 87. Synthesis of tert-butyl (S)-4-(1-(3-cyanophenyl)-3-cyclopropyl-1H-pyrazolo[3,4-d]pyrimidin-4-yl)-3-methylpiperazine-1-carboxylate (Compound 357)

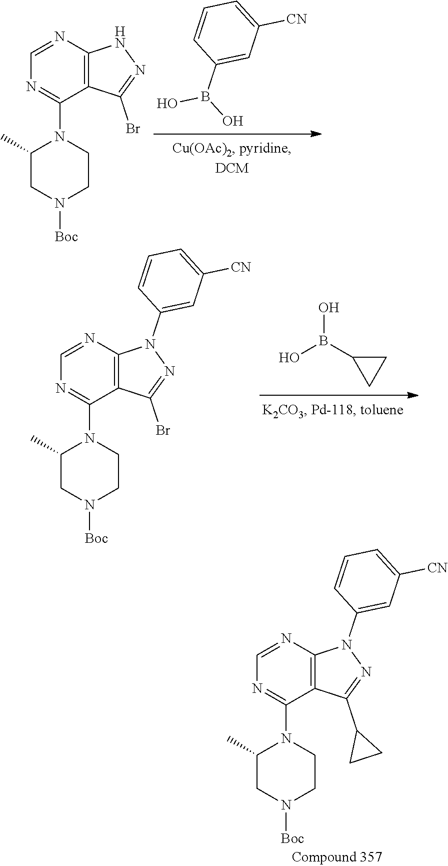 [SMILES: C[C@H]1CN(C(=O)OC(C)(C)C)CCN1c1ncnc2[nH]nc(Br)c12 ; N#Cc1cccc(B(O)O)c1 ; C[C@H]1CN(C(=O)OC(C)(C)C)CCN1c1ncnc2c1c(Br)nn2-c1cccc(C#N)c1 ; OB(O)C1CC1 ; C[C@H]1CN(C(=O)OC(C)(C)C)CCN1c1ncnc2c1c(C1CC1)nn2-c1cccc(C#N)c1]

### Step 1. tert-Butyl (S)-4-(3-bromo-1-(3-cyanophenyl)-1H-pyrazolo[3,4-d]pyrimidin-4-yl)-3-methylpiperazine-1-carboxylate

To a solution of tert-butyl (S)-4-(3-bromo-1H-pyrazolo[3,4-d]pyrimidin-4-yl)-3-methylpiperazine-1-carboxylate (500 mg, 1.3 mmol, prepared following the procedure outlined in compound 284) in DCM (20 mL) were added (3-cyanophenyl)boronic acid (370 mg, 2.5 mmol), Cu(OAc)₂ (560 mg, 3.1 mmol), pyridine (0.60 mL, 7.5 mmol) and 4 A molecular sieves (500 mg). The resulting mixture was stirred at room temperature overnight under O₂ atmosphere. The reaction was quenched with NH₄OH, diluted with DCM, and then filtered. The filtrate was extracted with DCM twice, the combined organic layers were washed with brine, dried over Na₂SO₄, filtered and concentrated. The residue was purified by flash column chromatography (silica gel, ethyl acetate in petroleum ether) to afford tert-butyl (S)-4-(3-bromo-1-(3-cyanophenyl)-1H-pyrazolo[3,4-d]pyrimidin-4-yl)-3-methylpiperazine-1-carboxylate (510 mg, 81%) as a light yellow solid. LC/MS ESI (m/z): 498,500 (M+H)⁺

### Step 2. tert-Butyl (S)-4-(1-(3-cyanophenyl)-3-cyclopropyl-1H-pyrazolo[3,4-d]pyrimidin-4-yl)-3-methylpiperazine-1-carboxylate

To a solution of tert-butyl (S)-4-(3-bromo-1-(3-cyanophenyl)-1H-pyrazolo[3,4-d]pyrimidin-4-yl)-3-methylpiperazine-1-carboxylate (200 mg, 0.40 mmol) in toluene (5 mL) were added cyclopropylboronic acid (52 mg, 0.60 mmol), K₂CO₃ (720 mg, 5.2 mmol) and Pd-118 (13 mg, 0.020 mmol). The resulting mixture was heated to 80° C. overnight. After being cooled down to room temperature, solvent was filtered. The filtrate was concentrated and purified by flash column chromatography (silica gel, 0˜30%, ethyl acetate in petroleum ether) to give crude product which was further purified by prep-HPLC to afford tert-butyl (S)-4-(1-(3-cyanophenyl)-3-cyclopropyl-1H-pyrazolo[3,4-d]pyrimidin-4-yl)-3-methylpiperazine-1-carboxylate (110 mg, 58%) as a white solid. LC/MS ESI (m/z): 460 (M+H)⁺. ¹H NMR (400 MHz, CD₃OD) δ 8.64 (s, 1H), 8.55-8.51 (m, 1H), 8.43 (s, 1H), 7.69-7.62 (m, 2H), 5.02 (s, 1H), 4.38 (d, J=13.1 Hz, 1H), 4.15 (d, J=11.8 Hz, 1H), 4.00-3.95 (m, 1H), 3.63-3.55 (m, 1H), 3.27-3.01 (m, 2H), 2.20-2.14 (m, 1H), 1.50 (s, 9H), 1.33 (d, J=6.6 Hz, 3H), 1.29-1.25 (m, 2H), 1.19-1.15 (m, 2H).

### Example 88. Synthesis of 1,1,1-trifluoro-2-methylpropan-2-yl (S)-4-(7-cyclohexyl-5-(pyrrolidin-1-yl)-7H-pyrrolo[2,3-d]pyrimidin-4-yl)-3-methylpiperazine-1-carboxylate (Compound 359)

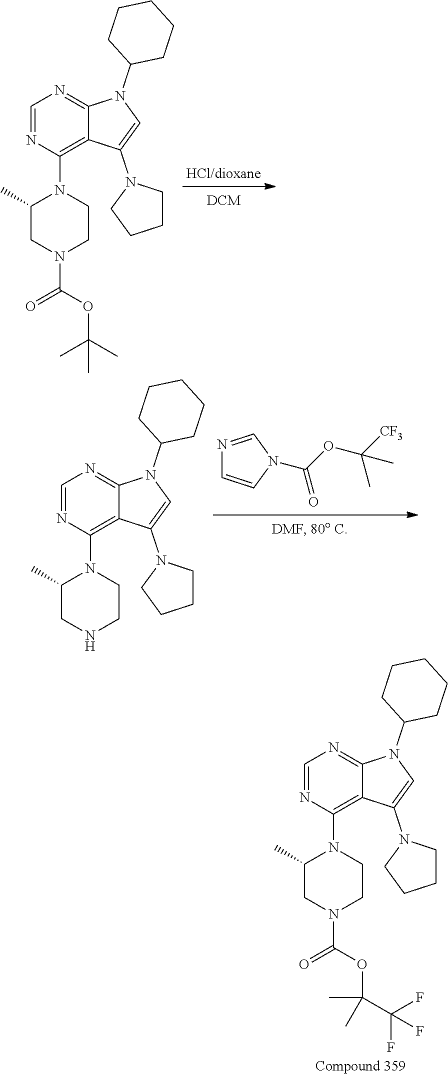

### Step 1. (S)-7-Cyclohexyl-4-(2-methylpiperazin-1-yl)-5-(pyrrolidin-1-yl)-7H-pyrrolo[2,3-d]pyrimidine

To a solution of tert-butyl (S)-4-(7-cyclohexyl-5-(pyrrolidin-1-yl)-7H-pyrrolo[2,3-d]pyrimidin-4-yl)-3-methylpiperazine-1-carboxylate (50 mg, 0.11 mmol, prepared following the procedure of compound 334, step 1) in DCM (1 mL) was added HCl/dioxane (1.0 mL, 4.0 M). The resulting mixture was stirred at room temperature overnight. After removal of solvent under reduced pressure, the residue was used in the next step directly. (S)-7-cyclohexyl-4-(2-methylpiperazin-1-yl)-5-(pyrrolidin-1-yl)-7H-pyrrolo[2,3-d]pyrimidine (30 mg, 74%). LC/MS ESI (m/z): 369 (M+H)⁺.

### Step 2. 1,1,1-Trifluoro-2-methylpropan-2-yl (S)-4-(7-cyclohexyl-5-(pyrrolidin-1-yl)-7H-pyrrolo[2,3-d]pyrimidin-4-yl)-3-methylpiperazine-1-carboxylate

To a solution of (S)-7-cyclohexyl-4-(2-methylpiperazin-1-yl)-5-(pyrrolidin-1-yl)-7H-pyrrolo[2,3-d]pyrimidine (30 mg, 0.080 mmol) in DMF (0.5 mL) were added 1,1,1-trifluoro-2-methylpropan-2-yl 1H-imidazole-1-carboxylate (88 mg, 0.40 mmol, prepared following the procedure of compound 247, step 1). The reaction mixture was stirred at 80° C. overnight under N₂. The reaction was quenched with water, extracted with EtOAc twice, the combined organic layers were washed with brine, dried over Na₂SO₄, filtered and concentrated. The residue was purified by prep-HPLC to afford 1,1,1-trifluoro-2-methylpropan-2-yl (S)-4-(7-cyclohexyl-5-(pyrrolidin-1-yl)-7H-pyrrolo[2,3-d]pyrimidin-4-yl)-3-methylpiperazine-1-carboxylate (2.6 mg, 6.2% yield) as a white solid. LC/MS ESI (m/z): 523 (M+H)⁺. ¹H NMR (400 MHz, CDCl₃) δ 8.32 (s, 1H), 6.54 (s, 1H), 5.10-4.88 (m, 1H), 4.78-4.58 (m, 1H), 4.27-3.94 (m, 2H), 3.94-3.77 (m, 1H), 3.45-3.21 (m, 2H), 3.18-2.93 (m, 3H), 2.91-2.71 (m, 2H), 2.09-1.99 (m, 2H), 1.99-1.82 (m, 6H), 1.80-1.74 (m, 1H), 1.69-1.61 (m, 2H), 1.58 (s, 6H), 1.55-1.46 (m, 2H), 1.27-1.23 (m, 1H), 1.11 (d, J=6.6 Hz, 3H). ¹⁹F NMR (377 MHz, CDCl₃) δ −83.86 (d, J=36.1 Hz).

### Example 89. Synthesis of tert-butyl (R)-2-methyl-4-(5-(pyrrolidin-1-yl)-7-(3,4,5-trifluorophenyl)-7H-pyrrolo[2,3-d]pyrimidin-4-yl)piperazine-1-carboxylate (Compound 362)

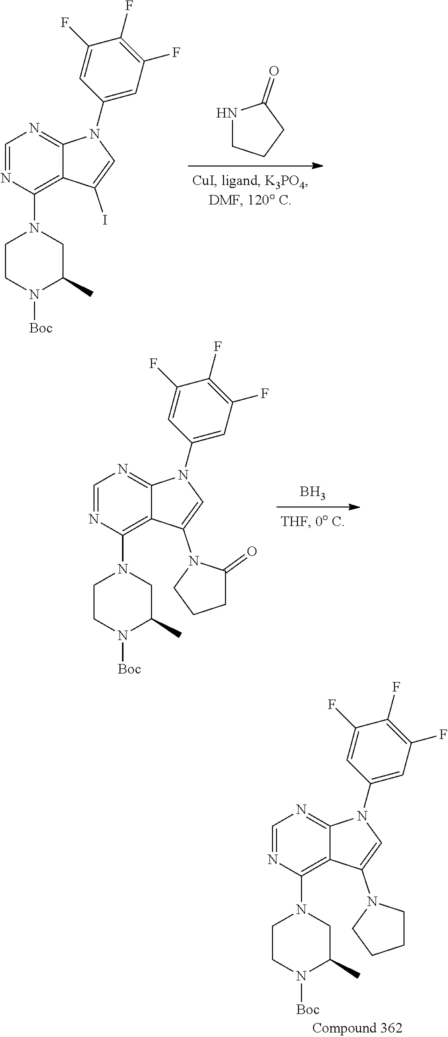

### Step 1. tert-Butyl (R)-2-methyl-4-(5-(2-oxopyrrolidin-1-yl)-7-(3,4,5-trifluorophenyl)-7H-pyrrolo[2,3-d]pyrimidin-4-yl)piperazine-1-carboxylate

To the solution of tert-butyl (R)-4-(5-iodo-7-(3,4,5-trifluorophenyl)-7H-pyrrolo[2,3-d]pyrimidin-4-yl)-2-methylpiperazine-1-carboxylate (300 mg, 0.52 mmol, prepared following a similar procedure outlined in compound 356) in DMF (2 mL) were added CuI (49 mg, 0.26 mmol), K₃PO₄ (330 mg, 1.6 mmol), trans-cyclohexane-1,2-diamine (0.020 mL, 0.16 mmol) and pyrrolidin-2-one (0.080 mL, 1.1 mmol) respectively. The resulting reaction mixture was stirred at 120° C. under N₂ overnight. After being cooled down to room temperature, the reaction mixture was diluted with ice water, then it was extracted with EtOAc twice, the combined organic layers were washed with and brine, dried over Na₂SO₄, filtered, and concentrated under reduced pressure. The crude product was purified by flash column chromatography (silica gel, 0˜50%, ethyl acetate in petroleum ether) to afford tert-butyl (R)-2-methyl-4-(5-(2-oxopyrrolidin-1-yl)-7-(3,4,5-trifluorophenyl)-7H-pyrrolo[2,3-d]pyrimidin-4-yl)piperazine-1-carboxylate (150 mg, 54%) as yellow solid. LC/MS ESI (m/z): 531 (M+H)⁺.

### Step 2. tert-Butyl (R)-2-methyl-4-(5-(pyrrolidin-1-yl)-7-(3,4,5-trifluorophenyl)-7H-pyrrolo[2,3-d]pyrimidin-4-yl)piperazine-1-carboxylate

To the solution of tert-butyl (R)-2-methyl-4-(5-(2-oxopyrrolidin-1-yl)-7-(3,4,5-trifluorophenyl)-7H-pyrrolo[2,3-d]pyrimidin-4-yl)piperazine-1-carboxylate (140 mg, 0.26 mmol) in THE (2 mL) was added BH₃ (5.0 mL, 1.0M in THF) at 0° C. under N₂. The resulting reaction mixture was stirred at 0° C. for 1 h. The reaction mixture was quenched with ice water, extracted with EtOAc twice. The combined organic layers were dried over Na₂SO₄, filtered, and concentrated under reduced pressure. The crude product was purified by prep-HPLC to obtain tert-butyl (R)-2-methyl-4-(5-(pyrrolidin-1-yl)-7-(3,4,5-trifluorophenyl)-7H-pyrrolo[2,3-d]pyrimidin-4-yl)piperazine-1-carboxylate (30 mg, 23%) as yellow solid. LC/MS ESI (m/z): 517 (M+H)⁺. ¹H NMR (400 MHz, CDCl₃) δ 8.36 (s, 1H), 7.54-7.48 (m, 2H), 6.64 (s, 1H), 4.76 (d, J=12.8 Hz, 1H), 4.41-4.34 (m, 2H), 3.92 (d, J=13.3 Hz, 1H), 3.35-3.20 (m, 4H), 3.00-2.93 (m, 1H), 2.88-2.82 (m, 2H), 2.01-1.97 (m, 4H), 1.49 (s, 9H), 1.09 (d, J=6.8 Hz, 3H).

### Example 90. Synthesis of tert-butyl (3S)-4-(7-(4-cyanopyridin-2-yl)-5-(2,2-difluorocyclopropyl)-7H-pyrrolo[2,3-d]pyrimidin-4-yl)-3-methylpiperazine-1-carboxylate (Compound 365)

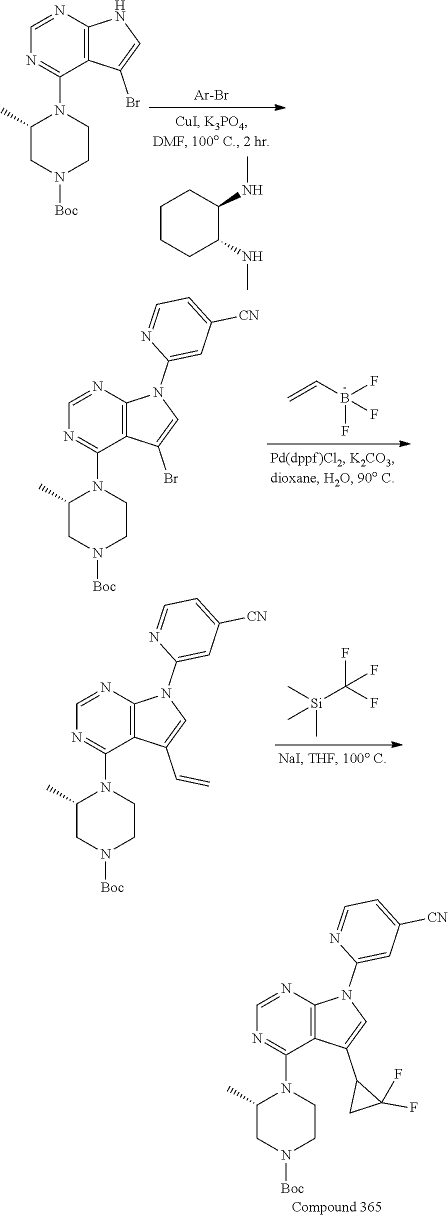

### Step 1. tert-Butyl (S)-4-(5-bromo-7-(4-cyanopyridin-2-yl)-7H-pyrrolo[2,3-d]pyrimidin-4-yl)-3-methylpiperazine-1-carboxylate

To a solution of tert-butyl (S)-4-(5-bromo-7H-pyrrolo[2,3-d]pyrimidin-4-yl)-3-methylpiperazine-1-carboxylate (900 mg, 2.3 mmol, prepared following the analogous procedure of compound 399) in DMF (5 mL) was added 2-bromoisonicotinonitrile (1.7 g, 9.1 mmol), trans-N,N′-dimethylcyclohexane-1,2-diamine (650 mg, 4.6 mmol), CuI (430 mg, 2.3 mmol) and K₃PO₄ (1.5 g, 6.8 mmol). The resulting mixture was heated at 100° C. for 1 h. After cooling down to room temperature, the reaction was partitioned between EtOAc and water, organic layer was separated, aqueous layer was extracted with EtOAc twice, the combined organic layers were washed with brine, dried over Na₂SO₄, filtered and concentrated, the residue was purified by flash column chromatography (silica gel, 0-25%, ethyl acetate in petroleum ether) to afford tert-butyl (S)-4-(5-bromo-7-(4-cyanopyridin-2-yl)-7H-pyrrolo[2,3-d]pyrimidin-4-yl)-3-methylpiperazine-1-carboxylate (800 mg, 70%) as a solid. LC/MS ESI (m/z): 498 (M+H)⁺.

### Step 2. tert-Butyl (S)-4-(7-(4-cyanopyridin-2-yl)-5-vinyl-7H-pyrrolo[2,3-d]pyrimidin-4-yl)-3-methylpiperazine-1-carboxylate

To a solution of tert-butyl (S)-4-(5-bromo-7-(4-cyanopyridin-2-yl)-7H-pyrrolo[2,3-d]pyrimidin-4-yl)-3-methylpiperazine-1-carboxylate (500 mg, 1.0 mmol) in dioxane (5 mL) and H₂O (1 mL) was added potassium trifluoro(vinyl)borate (270 mg, 2.0 mmol), K₂CO₃ (410 mg, 3.0 mmol) and Pd(dppf)Cl₂ (73 mg, 0.10 mmol). The resulting mixture was heated at 90° C. overnight under N₂. After cooling down to room temperature, the solvent was removed and the residue was purified by flash column chromatography (silica gel, 0-20%, ethyl acetate in petroleum ether) to afford tert-butyl (S)-4-(7-(4-cyanopyridin-2-yl)-5-vinyl-7H-pyrrolo[2,3-d]pyrimidin-4-yl)-3-methylpiperazine-1-carboxylate (260 mg, 58%) as a solid. LC/MS ESI (m/z): 446 (M+H)⁺.

### Step 3. tert-Butyl (3S)-4-(7-(4-cyanopyridin-2-yl)-5-(2,2-difluorocyclopropyl)-7H-pyrrolo[2,3-d]pyrimidin-4-yl)-3-methylpiperazine-1-carboxylate

To a solution of tert-butyl (S)-4-(7-(4-cyanopyridin-2-yl)-5-vinyl-7H-pyrrolo[2,3-d]pyrimidin-4-yl)-3-methylpiperazine-1-carboxylate (100 mg, 0.22 mmol) in THE (5 mL) was added trimethyl(trifluoromethyl)silane (380 mg, 2.6 mmol), NaI (130 mg, 0.88 mmol). The resulting mixture was heated at 100° C. for 2 hours in a microwave. After cooling down to room temperature, the solvent was removed and the residue was purified by flash column chromatography (silica gel, 0˜30%, ethyl acetate in petroleum ether) to afford tert-butyl (3S)-4-(7-(4-cyanopyridin-2-yl)-5-(2,2-difluorocyclopropyl)-7H-pyrrolo[2,3-d]pyrimidin-4-yl)-3-methylpiperazine-1-carboxylate (90 mg, 81%) as a solid, 90 mg of which was further purified by prep-HPLC to afford 65 mg of white solid. LC/MS ESI (m/z): 496 (M+H)⁺. ¹H NMR (400 MHz, CD₃OD) δ 9.24-9.18 (m, 1H), 8.70-8.66 (m, 1H), 8.52 (d, J=6.0 Hz, 1H), 8.13 (d, J=11.6 Hz, 1H), 7.60-7.55 (m, 1H), 4.52-4.44 (m, 1H), 4.08-3.97 (m, 1H), 3.85-3.02 (m, 6H), 2.05-1.75 (m, 2H), 1.50 (s, 9H), 1.26-1.14 (m, 3H).

The following compound was prepared by a procedure analogous to the synthesis of compound 365 using the appropriate aryl halide.

| Cmpd | | |
|---|---|---|
| No. | Chemical Name | LCMS and ¹H NMR |
| 376 | tert-butyl (3S)-4-(7-(3- | ¹H NMR(400 MHz, CD₃OD) δ 8.39 (d, J = 8.6 Hz, 1H), |
| | cyanophenyl)-5-(2,2- | 8.24 (m, 1H), 8.11-8.07 (m, 1H), 7.72 (m, J = 7.8 Hz, |
| | difluorocyclopropyl)-7H- | 2H), 7.60 (s, 1H), 4.52 (m, 1H), 4.03 (m, 1H), 3.88-3.39 |
| | pyrrolo[2,3-d]pyrimidin-4-yl)-3- | (m, 5H), 3.14-3.08 (m, 1H), 2.06-1.86 (m, 2H), 1.50 (s, |
| | methylpiperazine-1-carboxylate | 9H), 1.24 (m, 1H), 1.15 (m, J = 6.6 Hz, 2H). |

### Example 91. Synthesis of tert-butyl (S)-4-(7-(3-chlorophenyl)-5-(diethylamino)-7H-pyrrolo[2,3-d]pyrimidin-4-yl)-3-methylpiperazine-1-carboxylate (Compound 366)

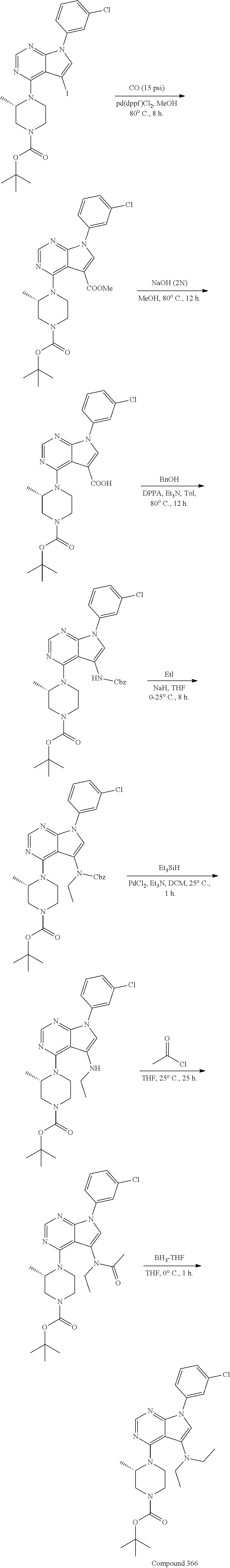

### Step 1. Methyl (S)-4-(4-(tert-butoxycarbonyl)-2-methylpiperazin-1-yl)-7-(3-chlorophenyl)-7H-pyrrolo[2,3-d]pyrimidine-5-carboxylate

To a solution of tert-butyl (S)-4-(7-(3-chlorophenyl)-5-iodo-7H-pyrrolo[2,3-d]pyrimidin-4-yl)-3-methylpiperazine-1-carboxylate (100 mg, 0.18 mmol, prepared following an analogous procedure to compound 114 in MeOH (2.0 mL) was added Pd(dppf)Cl₂ (13 mg, 0.018 mmol) at 25° C. The mixture was degassed with CO three times. The mixture was heated at 80° C. under CO (15 psi) for 8 hrs. The mixture was poured into H₂O (20 mL) and extracted with EtOAc twice. The combined organic phases were washed with brine, dried over Na₂SO₄, filtered and concentrated in vacuo. The methyl (S)-4-(4-(tert-butoxycarbonyl)-2-methylpiperazin-1-yl)-7-(3-chlorophenyl)-7H-pyrrolo[2,3-d]pyrimidine-5-carboxylate (70 mg) was obtained as a brown solid. LC/MS ESI (m/z): 486 (M+H)⁺.

### Step 2. (S)-4-(4-(tert-Butoxycarbonyl)-2-methylpiperazin-1-yl)-7-(3-chlorophenyl)-7H-pyrrolo[2,3-d]pyrimidine-5-carboxylic acid

To a solution of methyl (S)-4-(4-(tert-butoxycarbonyl)-2-methylpiperazin-1-yl)-7-(3-chlorophenyl)-7H-pyrrolo[2,3-d]pyrimidine-5-carboxylate in MeOH (10 mL) was added NaOH (10 mL, 2.0N) at 25° C. The mixture was stirred at 80° C. for 12 hrs. The pH was adjusted to 2 with dilute HCl (1 N). The mixture was extracted with DCM (20 mL×4) and concentrated in vacuo. The crude product was used in next step directly.

### Step 3. tert-Butyl (S)-4-(5-(((benzyloxy)carbonyl)amino)-7-(3-chlorophenyl)-7H-pyrrolo[2,3-d]pyrimidin-4-yl)-3-methylpiperazine-1-carboxylate

To a solution of tert-butyl (S)-4-(7-(3-chlorophenyl)-5-(diethylamino)-7H-pyrrolo[2,3-d]pyrimidin-4-yl)-3-methylpiperazine-1-carboxylate (100 mg, 0.21 mmol) in toluene (10 mL) were added benzyl alcohol (0.026 mL, 0.25 mmol), triethylamine (0.032 mL, 0.23 mmol) and DPPA (0.050 mL, 0.23 mmol) at 25° C. The mixture was degassed with N₂ three times. The mixture was stirred at 80° C. for 12 hrs. The mixture was then cooled to 25° C., poured into H₂O (20 mL) and extracted with EtOAc twice. The combined organic phases were washed with brine, dried over Na₂SO₄, filtered, and concentrated in vacuo. The crude product was purified by silica gel column (SiO₂, eluting with Pet ether:EtOAc=50:1 to 30:1, Rf=0.50) to afford tert-butyl (S)-4-(5-(((benzyloxy)carbonyl)amino)-7-(3-chlorophenyl)-7H-pyrrolo[2,3-d]pyrimidin-4-yl)-3-methylpiperazine-1-carboxylate (100 mg) as a brown solid. LC/MS ESI (m/z): 577 (M+H)⁺.

### Step 4. tert-Butyl (S)-4-(5-(((benzyloxy)carbonyl)(ethyl)amino)-7-(3-chlorophenyl)-7H-pyrrolo[2,3-d]pyrimidin-4-yl)-3-methylpiperazine-1-carboxylate

To a solution of tert-butyl (S)-4-(5-(((benzyloxy)carbonyl)amino)-7-(3-chlorophenyl)-7H-pyrrolo[2,3-d]pyrimidin-4-yl)-3-methylpiperazine-1-carboxylate (50 mg, 0.087 mmol) in THE (2.0 mL) was added NaH (3.6 mg, 0.091 mmol) at 0° C. The mixture was stirred at 0° C.˜25° C. for 1 hr. Then, EtI (0.0070 mL, 0.091 mmol) was added to the mixture at 25° C. The mixture was stirred at 25° C. for 8 h. The mixture was poured into H₂O (20 mL) and extracted with EtOAc twice. The combine organic phases were washed with brine, dried over Na₂SO₄, filtered, and concentrated in vacuo. tert-Butyl (S)-4-(5-(((benzyloxy)carbonyl)(ethyl)amino)-7-(3-chlorophenyl)-7H-pyrrolo[2,3-d]pyrimidin-4-yl)-3-methylpiperazine-1-carboxylate (50 mg) was obtained as a brown solid which was used in next step directly. LC/MS ESI (m/z): 605 (M+H)⁺.

### Step 5. tert-Butyl (S)-4-(7-(3-chlorophenyl)-5-(ethylamino)-7H-pyrrolo[2,3-d]pyrimidin-4-yl)-3-methylpiperazine-1-carboxylate

To a solution of tert-butyl (S)-4-(5-(((benzyloxy)carbonyl)(ethyl)amino)-7-(3-chlorophenyl)-7H-pyrrolo[2,3-d]pyrimidin-4-yl)-3-methylpiperazine-1-carboxylate (150 mg, 0.25 mmol) in DCM (5 mL) were added TEA (0.028 mL, 0.20 mmol), Et₃SiH (0.16 mL, 0.99 mmol), and PdCl₂ (44 mg, 0.25 mmol) at 25° C. The mixture was stirred at 25° C. for 0.5 hrs. After the LCMS showed that tert-butyl (S)-4-(5-(((benzyloxy)carbonyl)(ethyl)amino)-7-(3-chlorophenyl)-7H-pyrrolo[2,3-d]pyrimidin-4-yl)-3-methylpiperazine-1-carboxylate was consumed, the mixture was filtered and the product in DCM was used directly in the next step. LC/MS ESI (m/z): 471 (M+H)⁺.

### Step 6. tert-Butyl (S)-4-(7-(3-chlorophenyl)-5-(N-ethylacetamido)-7H-pyrrolo[2,3-d]pyrimidin-4-yl)-3-methylpiperazine-1-carboxylate

Acetyl chloride (0.088 mL, 1.2 mmol) was added to tert-butyl (S)-4-(7-(3-chlorophenyl)-5-(ethylamino)-7H-pyrrolo[2,3-d]pyrimidin-4-yl)-3-methylpiperazine-1-carboxylate (120 mg, 0.25 mmol) at 25° C. The mixture was stirred at 25° C. for 12 h. The mixture was poured into H₂O (20 mL) and extracted with EtOAc twice. The combined organic phases were washed with brine, dried over Na₂SO₄, filtered and concentrated in vacuo. The crude product was purified by Prep-HPLC and tert-butyl (S)-4-(7-(3-chlorophenyl)-5-(N-ethylacetamido)-7H-pyrrolo[2,3-d]pyrimidin-4-yl)-3-methylpiperazine-1-carboxylate (30 mg) was obtained as a white solid. LC/MS ESI (m/z): 513 (M+H)⁺.

### Step 7. tert-Butyl (S)-4-(7-(3-chlorophenyl)-5-(diethylamino)-7H-pyrrolo[2,3-d]pyrimidin-4-yl)-3-methylpiperazine-1-carboxylate

To a solution of tert-butyl (S)-4-(7-(3-chlorophenyl)-5-(N-ethylacetamido)-7H-pyrrolo[2,3-d]pyrimidin-4-yl)-3-methylpiperazine-1-carboxylate (13 mg, 0.025 mmol) in THE (2.0 mL) was added BH₃-THF (0.050 mL, 1.0M in THF) at 0° C. under N₂. The mixture was stirred at 0° C. for 2 h. MeOH (5.0 m1) was added to the mixture at 0° C. and stirred for 0.5 hr. The mixture was poured into H₂O (100 mL) and extracted with EtOAc twice. The combined organic phases were washed with brine, dried over Na₂SO₄, filtered, and concentrated in vacuo. The crude product was purified by prep-HPLC and tert-butyl (S)-4-(7-(3-chlorophenyl)-5-(diethylamino)-7H-pyrrolo[2,3-d]pyrimidin-4-yl)-3-methylpiperazine-1-carboxylate (2.8 mg, 24%) was obtained as a white solid. LC/MS ESI (m/z): 499 (M+H)^{+ 1}H NMR (400 MHz, CDCl₃) δ 8.39 (s, 1H), 7.71 (s, 1H), 7.66 (d, J=7.8 Hz, 1H), 7.43 (t, J=8.1 Hz, 1H), 7.30 (d, J=7.4 Hz, 1H), 6.80 (s, 1H), 5.14 (s, 1H), 4.33-3.80 (m, 3H), 3.58-3.24 (m, 2H), 3.20-2.97 (m, 5H), 1.56 (s, 9H), 1.18 (s, 3H), 1.02 (t, J=7.0 Hz, 6H).

The following compound was prepared by a procedure analogous to the synthesis of compound 366, using the appropriate piperazine or amine.

| Cmpd | | |
|---|---|---|
| No. | Chemical Name | LCMS and ¹H NMR |
| 375 | tert-butyl (R)-4-(7-(3- | LC/MS ESI (m/z): 499 (M + H)⁺. ¹H NMR (400 MHz, |
| | chlorophenyl)-5-(diethylamino)- | CDCl₃) δ 8.37 (s, 1H), 7.73 (t, J = 2.0 Hz, 1H), 7.67 (ddd, |
| | 7H-pyrrolo[2,3-d]pyrimidin-4- | J = 8.1, 2.0, 0.9 Hz, 1H), 7.42 (t, J = 8.1 Hz, 1H), 7.29-7.27 |
| | yl)-2-methylpiperazine-1- | (m, 1H), 6.78 (s, 1H), 4.93 (d, J = 12.6 Hz, 1H), 4.49 (d, J = |
| | carboxylate | 13.2 Hz, 1H), 4.36 (s, 1H), 3.91 (d, J = 13.2 Hz, 1H), 3.38 |
| | | (td, J = 12.9, 3.4 Hz, 1H), 3.26-3.07 (m, 5H), 2.95 (td, J = |
| | | 12.4, 3.5 Hz, 1H), 1.50 (s, 9H), 1.15 (d, J = 6.8 Hz, 3H), |
| | | 1.03 (t, J = 6.6 Hz, 6H). |
| 423 | tert-butyl (S)-4-(7-(4- | LC/MS ESI (m/z): 505 (M + H)⁺. ¹H NMR (400 MHz, |
| | cyanopyridin-2-yl)-5- | CDCl₃) δ 9.38 (s, 1H), 8.57 (d, J = 5.0 Hz, 1H), 8.43 |
| | (ethyl(propyl)amino)-7H- | (s, 1H), 7.60 (s, 1H), 7.29 (dd, J = 5.0, 1.0 Hz, 1H), |
| | pyrrolo[2,3-d]pyrimidin-4-yl)-3- | 5.60-4.98 (m, 1H), 4.66-3.77 (m, 3H), 3.62-2.56 (m, 7H), |
| | methylpiperazine-1-carboxylate | 1.61-1.55 (m, 2H), 1.50 (s, 9H), 1.15 (s, 3H), 0.95 |
| | | (t, J = 6.9 Hz, 3H), 0.89 (t, J = 7.3 Hz, 3H). |

### Example 92. Synthesis of 2,2,2-trifluoroethyl (S)-4-(7-(4-cyanopyridin-2-yl)-5-cyclopropyl-7H-pyrrolo[2,3-d]pyrimidin-4-yl)-3-methylpiperazine-1-carboxylate (Compound 368)

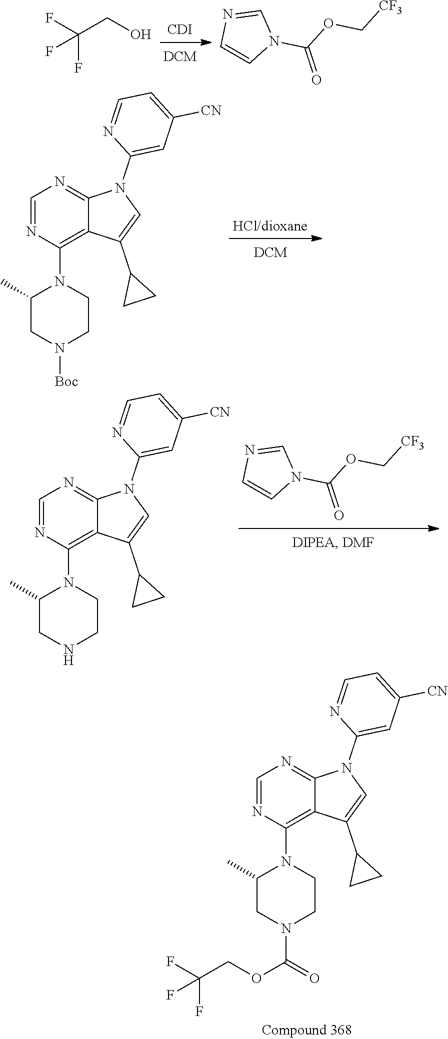

### Step 11. 2,2,2-Trifluoroethyl 1H-imidazole-1-carboxylate

To a solution of 2,2,2-trifluoroethan-1-ol (500 mg, 5.0 mmol) in DCM (5 mL) was added di(1H-imidazol-1-yl)methanone (980 mg, 6.0 mmol). The resulting mixture was stirred at rt overnight. The reaction was then quenched with water, extracted with DCM twice, the combined organic layers washed with brine, dried over Na₂SO₄, filtered and concentrated to afford 2,2,2-trifluoroethyl 1H-imidazole-1-carboxylate (650 mg, 67%) as a white solid. LC/MS ESI (m/z): 195 (M+H)⁺.

### Step 2. (S)-2-(5-Cyclopropyl-4-(2-methylpiperazin-1-yl)-7H-pyrrolo[2,3-d]pyrimidin-7-yl)isonicotinonitrile

To a 0° C. solution of tert-butyl (S)-4-(7-(4-cyanopyridin-2-yl)-5-cyclopropyl-7H-pyrrolo[2,3-d]pyrimidin-4-yl)-3-methylpiperazine-1-carboxylate (100 mg, 0.22 mmol, prepared following a similar procedure outlined in compound 259) in DCM (5 m1) was added HCl (2.0 mL, 4.0M in dioxane). The resulting mixture was stirred at the same temperature for 3 h. The reaction was quenched with NaHCO₃ (aq.), extracted with DCM twice, the combined organic layers washed with brine, dried over Na₂SO₄, filtered and concentrated. The residue was used in the next step directly. (S)-2-(5-cyclopropyl-4-(2-methylpiperazin-1-yl)-7H-pyrrolo[2,3-d]pyrimidin-7-yl)isonicotinonitrile. LC/MS ESI (m/z): 360 (M+H)⁺.

### Step 3. 2,2,2-Trifluoroethyl (S)-4-(7-(4-cyanopyridin-2-yl)-5-cyclopropyl-7H-pyrrolo[2,3-d]pyrimidin-4-yl)-3-methylpiperazine-1-carboxylate

To a solution of (S)-2-(5-cyclopropyl-4-(2-methylpiperazin-1-yl)-7H-pyrrolo[2,3-d]pyrimidin-7-yl)isonicotinonitrile (50 mg, 0.14 mmol) in DMF (3 mL) was added 2,2,2-trifluoroethyl 1H-imidazole-1-carboxylate (32 mg, 0.17 mmol) and DIPEA (0.040 mL, 0.22 mmol). The resulting mixture was stirred at 50° C. overnight. After cooling down to room temperature, the reaction was quenched with water, extracted with EtOAc twice, the combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford 2,2,2-trifluoroethyl (S)-4-(7-(4-cyanopyridin-2-yl)-5-cyclopropyl-7H-pyrrolo[2,3-d]pyrimidin-4-yl)-3-methylpiperazine-1-carboxylate (15 mg, 23%) as a white solid. LC/MS ESI (m/z): 486 (M+H)⁺. ¹H NMR (400 MHz, CD₃OD) δ 9.21-9.17 (s, 1H), 8.65 (d, J=5.0, 0.8 Hz, 1H), 8.47 (s, 1H), 7.86 (d, J=0.7 Hz, 1H), 7.53 (dd, J=5.0, 1.3 Hz, 1H), 4.76 (m, 1H), 4.63 (m, J=33.3 Hz, 2H), 4.12 (m, 1H), 3.93 (m, J=12.8 Hz, 2H), 3.64-3.56 (m, 1H), 3.55-3.44 (m, 1H), 3.36-3.24 (m, 1H), 2.09 (m, 1H), 1.22 (d, J=6.6 Hz, 3H), 1.07 (dt, J=4.7, 3.8 Hz, 2H), 0.90-0.84 (m, 1H), 0.79-0.72 (m, 1H).

The following compound was prepared analogous to the synthesis of compound 368 using the corresponding alcohol.

| Cmpd | | |
|---|---|---|
| No. | Chemical Name | LCMS and ¹H NMR |
| 318 | 1,1,1-trifluoro-2-methylpropan- | LC/MS ESI (m/z): 514.5 (M + H)⁺. ¹H NMR (400 MHz, |
| | 2-yl (S)-4-(7-(4-cyanopyridin-2- | CDCl₃) δ 9.32 (s, 1H), 8.61-8.56 (m, 1H), 8.51 (s, 1H), |
| | yl)-5-cyclopropyl-7H- | 7.79 (s, 1H), 7.37-7.32 (m, 1H), 4.79-4.66 (m, 1H), |
| | pyrrolo[2,3-d]pyrimidin-4-yl)-3- | 4.16-3.78 (m, 3H), 3.66-3.49 (m, 1H), 3.46-3.05 (m, 2H), |
| | methylpiperazine-1-carboxylate | 2.08-1.93 (m, 1H), 1.73 (d, J = 6.9 Hz, 6H), 1.24 (d, |
| | | J = 6.6 Hz, 3H), 1.07-0.98 (m, 2H), 0.90-0.74 (m, 2H). |

### Example 93. Synthesis of 2,2,2-trifluoroethyl 4-(7-(4-cyanopyridin-2-yl)-5-(2-fluorophenyl)-7H-pyrrolo[2,3-d]pyrimidin-4-yl)piperazine-1-carboxylate (Compound 373)

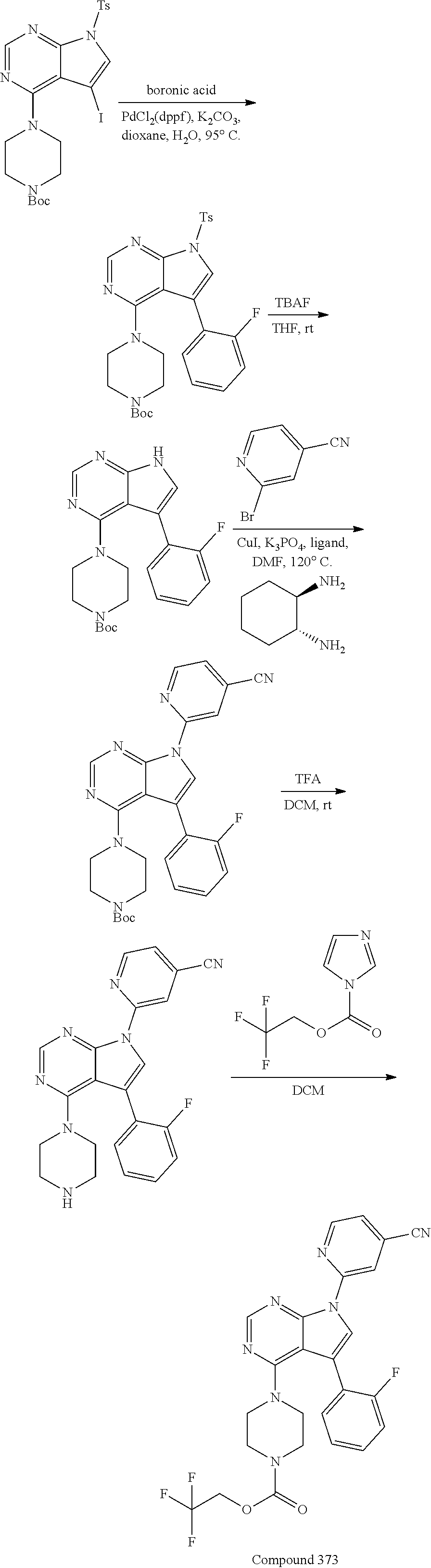

### Step 1. tert-Butyl-4-(5-(2-fluorophenyl)-7-tosyl-7H-pyrrolo[2,3-d]pyrimidin-4-yl)piperazine-1-carboxylate

A suspension of tert-butyl-4-[5-iodo-7-(4-methylbenzenesulfonyl)-7H-pyrrolo[2,3-d]pyrimidin-4-yl]piperazine-1-carboxylate (2.3 g, 4.0 mmol, prepared according to the procedure outlined in compound 146, (2-fluorophenyl)boronic acid (0.61 g, 4.4 mmol), K₂CO₃ (1.1 g, 8.0 mmol) and Pd(dppf)Cl₂ (0.29 g, 0.40 mmol) in dioxane-water (18 mL; 5.1 mixture) was stirred at 95° C. under N2 atmosphere for 18 h, after which the mixture was cooled to room temperature and concentrated. The residue was partitioned between water and EtOAc, organic layer was separated, aqueous layer was extracted by EtOAc twice. The combined organic layers were washed with brine, dried over anhydrous Na₂SO₄, filtered, and concentrated. The residue was used directly in the next step without further purification. LC/MS (ESI) (m/z): 552 (M+H)⁺

### Step 2. tert-Butyl-4-(5-(2-fluorophenyl)-7H-pyrrolo[2,3-d]pyrimidin-4-yl)piperazine-1-carboxylate

To a solution of tert-butyl-4-[5-(2-fluorophenyl)-7-(4-methylbenzenesulfonyl)-7H-pyrrolo[2,3-d]pyrimidin-4-yl]piperazine-1-carboxylate (1.8 g, 3.3 mmol) in THF (15 mL) was added 1.0M TBAF (6.5 mL, 6.5 mmol) and the resulting mixture stirred for 18 h. The reaction mixture was concentrated and partitioned between water and EtOAc. The organic layers were separated, and the aqueous layer was extracted with EtOAc twice. The combined organic phases were dried over anhydrous Na₂SO₄, filtered and concentrated. The residue was purified by column chromatography (silica gel, 0-20% MeOH in DCM) to give tert-butyl-4-[5-(2-fluorophenyl)-7H-pyrrolo[2,3-d]pyrimidin-4-yl]piperazine-1-carboxylate (1.2 g, 92%) as a white solid. LC/MS (ESI) (m/z): 398 (M+H)⁺

### Step 3. tert-Butyl-4-(7-(4-cyanopyridin-2-yl)-5-(2-fluorophenyl)-7H-pyrrolo[2,3-d]pyrimidin-4-yl)piperazine-1-carboxylate

A suspension of tert-butyl-4-[5-(2-fluorophenyl)-7H-pyrrolo[2,3-d]pyrimidin-4-yl]piperazine-1-carboxylate (1.2 g, 3.0 mmol), 2-bromopyridine-4-carbonitrile (0.66 g, 3.6 mmol), K₃PO₄ (1.3 g, 6.0 mmol), CuI (0.17 g, 0.90 mmol) and trans-1,2-diaminocyclohexane (0.10 g, 0.90 mmol) in DMF (30 mL) was stirred at 120° C. under N₂ atmosphere for 18 h. After cooling down to room temperature, the reaction mixture was partitioned between EtOAc and water, organic layer separated, and aqueous layer was extracted with EtOAc twice. The combined organic phases were washed with brine, dried over anhydrous Na₂SO₄, filtered and concentrated. The residue was purified by column chromatography on silica gel (0˜10% MeOH in DCM) to give tert-butyl-4-[7-(4-cyanopyridin-2-yl)-5-(2-fluorophenyl)-7H-pyrrolo[2,3-d]pyrimidin-4-yl]piperazine-1-carboxylate (1.0 g, 66%) as a white solid. LC/MS (ESI) (m/z): 500 (M+H)⁺

### Step 4. 2-(5-(2-Fluorophenyl)-4-(piperazin-1-yl)-7H-pyrrolo[2,3-d]pyrimidin-7-yl)isonicotinonitrile

To a solution of tert-butyl-4-[7-(4-cyanopyridin-2-yl)-5-(2-fluorophenyl)-7H-pyrrolo[2,3-d]pyrimidin-4-yl]piperazine-1-carboxylate (1.0 g, 2.0 mmol) in DCM (5.0 mL) was added TFA (4.5 mL, 60 mmol), the reaction was stirred at rt for 3 h. The mixture was then partitioned between EtOAc and saturated Na₂CO₃ solution. The organic layer was separated and the aqueous layer was extracted with EtOAc twice. The combined organic layers were washed with brine, dried over anhydrous Na₂SO₄, filtered, and concentrated. The residue was used directly in next step without further purification. LC/MS ESI (m/z): 400 (M+H)⁺

### Step 5. 2,2,2-Trifluoroethyl 4-(7-(4-cyanopyridin-2-yl)-5-(2-fluorophenyl)-7H-pyrrolo[2,3-d]pyrimidin-4-yl)piperazine-1-carboxylate

To a solution of 2,2,2-trifluoroethyl-1H-imidazole-1-carboxylate (58 mg, 0.30 mmol prepared following the procedure outlined in compound 254) in DCM (5 mL) was added a solution of 2-[5-(2-fluorophenyl)-4-(piperazin-1-yl)-7H-pyrrolo[2,3-d]pyrimidin-7-yl]pyridine-4-carbonitrile (80 mg, 0.20 mmol, prepared from alcohol and CDI), and the resulting mixture was stirred at room temperature overnight. The residue was suspended in water and DCM. The organic layer was separated and the aqueous layer was extracted by DCM twice. The combined organic phases were washed with brine, dried over anhydrous Na₂SO₄, filtered and concentrated. The residue was purified by prep-HPLC to give 2,2,2-trifluoroethyl 4-(7-(4-cyanopyridin-2-yl)-5-(2-fluorophenyl)-7H-pyrrolo[2,3-d]pyrimidin-4-yl)piperazine-1-carboxylate (18 mg, 17%) as a white solid. LC/MS (ESI) (m/z): 526 (M+H)^{+H NMR (}400 MHz, CDCl₃) δ 9.40 (s, 1H), 8.63 (d, J=5.0 Hz, 1H), 8.60 (s, 1H), 8.32 (s, 1H), 7.48 (td, J=7.5, 1.5 Hz, 1H), 7.39 (ddd, J=9.4, 6.1, 1.4 Hz, 2H), 7.29-7.19 (m, 2H), 4.45 (q, J=8.5 Hz, 2H), 3.29 (d, J=18.5 Hz, 8H).

### Example 94. Synthesis of tert-butyl (S)-4-(7-(3,5-difluoro-4-methylphenyl)-5-(pyrrolidin-1-yl)-7H-pyrrolo[2,3-d]pyrimidin-4-yl)-3-methylpiperazine-1-carboxylate (Compound 374)

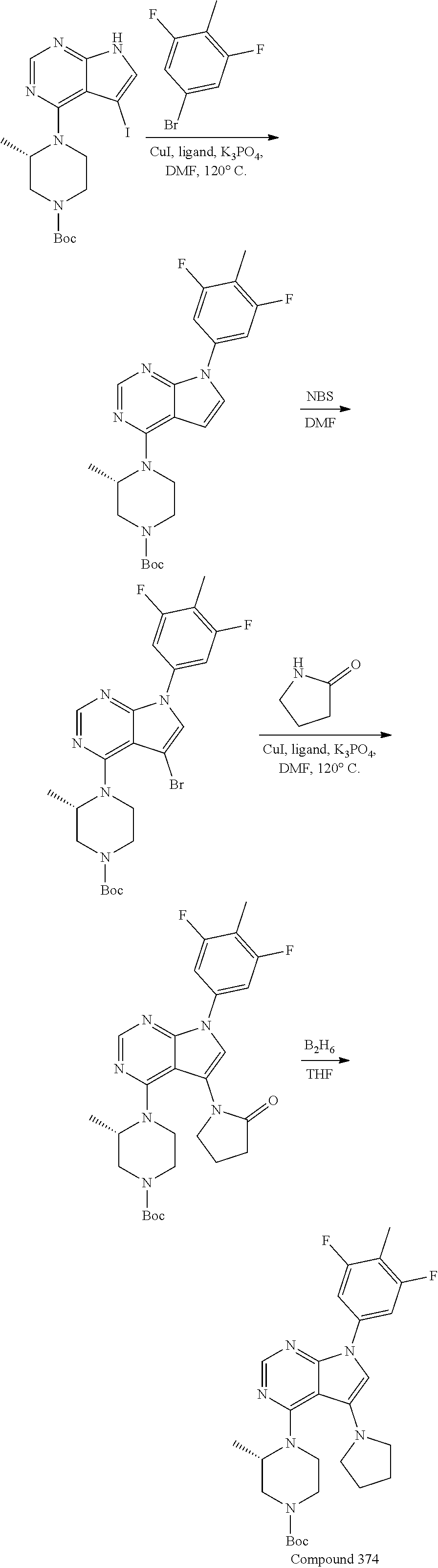

### Step 1. tert-Butyl (S)-4-(7-(3,5-difluoro-4-methylphenyl)-7H-pyrrolo[2,3-d]pyrimidin-4-yl)-3-methylpiperazine-1-carboxylate

A mixture of tert-butyl (S)-4-(5-iodo-7H-pyrrolo[2,3-d]pyrimidin-4-yl)-3-methylpiperazine-1-carboxylate (300 mg, 0.68 mmol), 5-bromo-1,3-difluoro-2-methylbenzene (280 mg, 1.4 mmol), CuI (64 mg, 0.34 mmol), trans-1,2-diaminocyclohexane (23 mg, 0.20 mmol) and K₃PO₄ (430 mg, 2.0 mmol) in DMF (10 mL) was heated at 120° C. for 12 hours. The reaction mixture was concentrated to a residue. The residue was purified by column chromatography (silica gel, 0˜30% EtOAc in Pet Ether) to give tert-butyl (S)-4-(7-(3,5-difluoro-4-methylphenyl)-7H-pyrrolo[2,3-d]pyrimidin-4-yl)-3-methylpiperazine-1-carboxylate (260 mg, 85%) as a yellow solid. LC/MS ESI (m/z): 444 (M+H)⁺.

### Step 2. tert-Butyl (S)-4-(5-bromo-7-(3,5-difluoro-4-methylphenyl)-7H-pyrrolo[2,3-d]pyrimidin-4-yl)-3-methylpiperazine-1-carboxylate

To a 0° C. mixture of tert-butyl (S)-4-(7-(3,5-difluoro-4-methylphenyl)-7H-pyrrolo[2,3-d]pyrimidin-4-yl)-3-methylpiperazine-1-carboxylate (200 mg, 0.45 mmol) in DMF (10 mL) was added NBS (96 mg, 0.54 mmol) in portions. The resulting mixture was stirred at room temperature for 1 hr. The reaction mixture diluted with water, extracted with EtOAc (50 mL×2). The combined layers were washed with brine, dried over Na₂SO₄ and concentrated. The residue was purified by column chromatography (silica gel, 0-20% EtOAc in Pet Ether) to give the tert-butyl (S)-4-(5-bromo-7-(3,5-difluoro-4-methylphenyl)-7H-pyrrolo[2,3-d]pyrimidin-4-yl)-3-methylpiperazine-1-carboxylate (100 mg, 42%) as a yellow solid. LC/MS ESI (m/z): 522, 524 (M+H)⁺.

### Step 3. tert-Butyl (S)-4-(7-(3,5-difluoro-4-methylphenyl)-5-(2-oxopyrrolidin-1-yl)-7H-pyrrolo[2,3-d]pyrimidin-4-yl)-3-methylpiperazine-1-carboxylate

A mixture of tert-butyl (S)-4-(5-bromo-7-(3,5-difluoro-4-methylphenyl)-7H-pyrrolo[2,3-d]pyrimidin-4-yl)-3-methylpiperazine-1-carboxylate (100 mg, 0.19 mmol), pyrrolidin-2-one (33 mg, 0.38 mmol), CuI (18 mg, 0.10 mmol), trans-1,2-diaminocyclohexane (6.6 mg, 0.060 mmol) and K₃PO₄ (120 mg, 0.57 mmol) in DMF (10 mL) was heated at 120° C. for 12 hours. The reaction mixture was concentrated and the residue was purified by column chromatography (silica gel, 0-100% EtOAc in Pet Ether) to give the tert-butyl (S)-4-(7-(3,5-difluoro-4-methylphenyl)-5-(2-oxopyrrolidin-1-yl)-7H-pyrrolo[2,3-d]pyrimidin-4-yl)-3-methylpiperazine-1-carboxylate (50 mg, 50%) as a yellow solid. LC/MS ESI (m/z): 527 (M+H)⁺.

### Step 4. tert-Butyl (S)-4-(7-(3,5-difluoro-4-methylphenyl)-5-(pyrrolidin-1-yl)-7H-pyrrolo[2,3-d]pyrimidin-4-yl)-3-methylpiperazine-1-carboxylate

To a 0° C. solution of tert-butyl (S)-4-(7-(3,5-difluoro-4-methylphenyl)-5-(2-oxopyrrolidin-1-yl)-7H-pyrrolo[2,3-d]pyrimidin-4-yl)-3-methylpiperazine-1-carboxylate (50 mg, 0.10 mmol) in THE (5 mL), B₂H₆ (2.0 mL, 1.0M in THF) was added dropwise. The resulting mixture was stirred at room temperature for 2 hrs. The reaction mixture was quenched with ice water, the resulting mixture was extracted with EtOAc (50 m1×2). The combined layers were washed with brine, dried over Na₂SO₄ and concentrated. The residue was purified by column chromatography on silica gel (0˜50% EtOAc in Pet Ether) to give the crude product, which was further purified by HPLC to give the tert-butyl (S)-4-(7-(3,5-difluoro-4-methylphenyl)-5-(pyrrolidin-1-yl)-7H-pyrrolo[2,3-d]pyrimidin-4-yl)-3-methylpiperazine-1-carboxylate (11 mg, 21%) as yellow solid. LC/MS ESI (m/z): 513 (M+H)⁺. ¹H NMR (400 MHz, CDCl₃) δ 8.37 (s, 1H), 7.34 (d, J=7.9 Hz, 2H), 6.67 (s, 1H), 4.97 (br, 1H), 4.29-3.83 (m, 3H), 3.50-3.09 (m, 4H), 2.91 (broad s, 3H), 2.22 (s, 3H), 1.99 (m, 4H), 1.49 (s, 9H), 1.14 (s, 3H).

### Example 95. Synthesis of 2,2,2-trifluoroethyl (R)-4-(7-(4-cyanopyridin-2-yl)-5-cyclopropyl-7H-pyrrolo[2,3-d]pyrimidin-4-yl)-2-methylpiperazine-1-carboxylate (Compound 377)

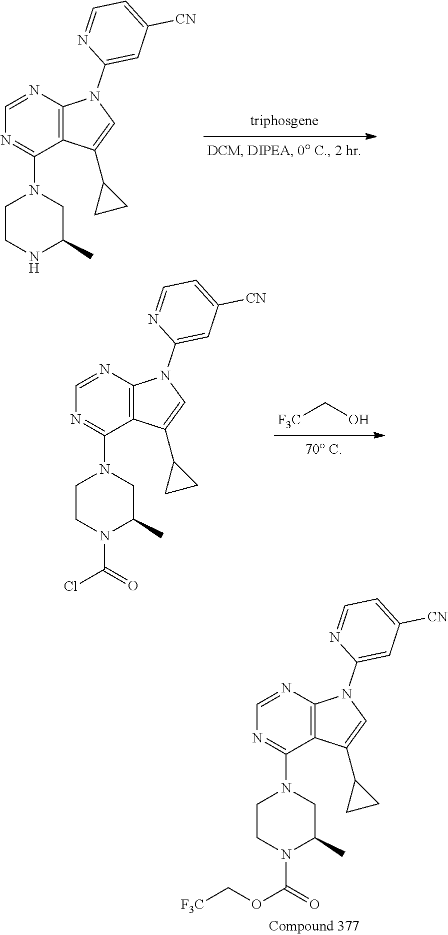 [SMILES: C[C@@H]1CN(c2ncnc3c2c(C2CC2)cn3-c2cc(C#N)ccn2)CCN1 ; C[C@@H]1CN(c2ncnc3c2c(C2CC2)cn3-c2cc(C#N)ccn2)CCN1C(=O)Cl ; OCC(F)(F)F ; C[C@@H]1CN(c2ncnc3c2c(C2CC2)cn3-c2cc(C#N)ccn2)CCN1C(=O)OCC(F)(F)F]

### Step 1. (R)-4-(7-(4-Cyanopyridin-2-yl)-5-cyclopropyl-7H-pyrrolo[2,3-d]pyrimidin-4-yl)-2-methylpiperazine-1-carbonyl chloride

To a 0° C. solution of (R)-2-(5-cyclopropyl-4-(3-methylpiperazin-1-yl)-7H-pyrrolo[2,3-d]pyrimidin-7-yl)isonicotinonitrile (40 mg, 0.11 mmol, prepared following the procedure outlined in compound 368) in DCM (5 mL) was added DIPEA (28 mg, 0.22 mmol), followed by a solution of triphosgene (22 mg, 0.070 mmol) in DCM (1 mL) dropwise. The resulting mixture was stirred at 0° C. for 2 hours under N₂. Solvent was removed and the residue was used in the next step directly. LC/MS ESI (m/z): 422 (M+H)⁺.

### Step 2. 2,2,2-Trifluoroethyl(R)-4-(7-(4-cyanopyridin-2-yl)-5-cyclopropyl-7H-pyrrolo[2,3-d]pyrimidin-4-yl)-2-methylpiperazine-1-carboxylate

A solution of (R)-4-(7-(4-cyanopyridin-2-yl)-5-cyclopropyl-7H-pyrrolo[2,3-d]pyrimidin-4-yl)-2-methylpiperazine-1-carbonyl chloride (0.11 mmol) in 2,2,2-trifluoroethan-1-ol (3 mL) was heated to 70° C. overnight under N₂. After cooling down to room temperature, the solvent was removed in vacuo and the residue purified by flash column chromatography (silica gel, 30% ethyl acetate in petroleum ether) and then by prep-HPLC to afford 2,2,2-trifluoroethyl(R)-4-(7-(4-cyanopyridin-2-yl)-5-cyclopropyl-7H-pyrrolo[2,3-d]pyrimidin-4-yl)-2-methylpiperazine-1-carboxylate (10 mg, 19% over 2 steps) as a white solid. LC/MS ESI (m/z): 486 (M+H)⁺. ¹H NMR (400 MHz, CDCl₃) δ 9.32 (s, 1H), 8.59 (dd, J=5.0, 0.7 Hz, 1H), 8.51 (s, 1H), 7.82 (d, J=0.7 Hz, 1H), 7.35 (dd, J=5.0, 1.3 Hz, 1H), 4.61-4.46 (m, 4H), 4.19-4.12 (m, 1H), 4.04 (d, J=12.8 Hz, 1H), 3.56-3.48 (m, 1H), 3.39 (dd, J=13.1, 3.9 Hz, 1H), 3.12 (td, J=12.5, 3.3 Hz, 1H), 2.04-1.98 (m, 1H), 1.27 (d, J=6.8 Hz, 3H), 1.09-1.03 (m, 2H), 1.01-0.95 (m, 1H), 0.75-0.69 (m, 1H).

The following compound was prepared by using an analogous protocol to compound 377 using the appropriate alcohol.

| Cmpd No. | Chemical Name | LCMS and ¹H NMR |
|---|---|---|
| 378 | 2,2,2-trifluoroethyl (R)-4-(7-(4- | LC/MS ESI (m/z): 540.6 (M + H)⁺. ¹H NMR (400 MHz, |
| | cyanopyridin-2-yl)-5-(2- | CDCl₃) δ 9.41 (s, 1H), 8.65-8.61 (m, 1H), 8.59 (s, 1H), |
| | fluorophenyl)-7H-pyrrolo[2,3- | 8.30 (s, 1H), 7.53-7.45 (m, 1H), 7.44-7.36 (m, 2H), 7.30- |
| | d]pyrimidin-4-yl)-2- | 7.26 (m, 1H), 7.25-7.19 (m, 1H), 4.60-4.23 (m, 3H), |
| | methylpiperazine-1-carboxylate | 3.94-3.82 (m, 1H), 3.72-3.59 (m, 1H), 3.58-3.43 (m, |
| | | 1H), 3.11-2.99 (m, 1H), 2.82-2.56 (m, 2H), 1.18 (d, J = |
| | | 6.8 Hz, 3H). |

### Example 96. Synthesis of tert-butyl (S)-4-(7-(4-cyanopyridin-2-yl)-5-morpholino-7H-pyrrolo[2,3-d]pyrimidin-4-yl)-3-methylpiperazine-1-carboxylate (Compound 380)

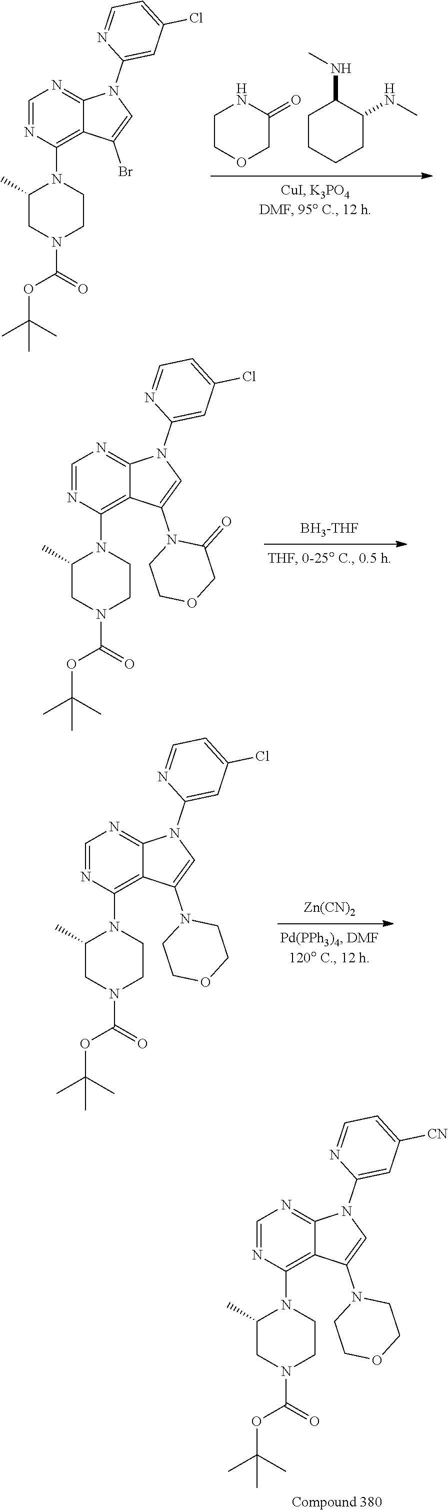

### Step 1. tert-Butyl (S)-4-(7-(4-chloropyridin-2-yl)-5-(3-oxomorpholino)-7H-pyrrolo[2,3-d]pyrimidin-4-yl)-3-methylpiperazine-1-carboxylate

To a solution of tert-butyl (S)-4-(5-bromo-7-(4-chloropyridin-2-yl)-7H-pyrrolo[2,3-d]pyrimidin-4-yl)-3-methylpiperazine-1-carboxylate (300 mg, 0.59 mmol, prepared according to the procedure outlined for compound 388) in DMF (8.0 mL) was added CuI (0.020 mL, 0.59 mmol), K₃PO₄ (250 mg, 1.2 mmol) and trans-N,N′-dimethyl-1,2-cyclohexyldiamine (170 mg, 1.2 mmol) at 25° C. The mixture was degassed under N₂ three times. The mixture was stirred at 95° C. for 16 h. The mixture was then cooled, poured into H₂O (100 mL) and extracted with EtOAc twice. The combined organic phases were washed with brine, dried over Na₂SO₄, filtered, and concentrated in vacuo. The crude product was purified by flash chromatography (silica gel, petroleum ether:EtOAc=20:1 to 10:1) to afford tert-butyl (S)-4-(7-(4-chloropyridin-2-yl)-5-(3-oxomorpholino)-7H-pyrrolo[2,3-d]pyrimidin-4-yl)-3-methylpiperazine-1-carboxylate (30 mg) as a yellow solid. LC/MS ESI (m/z): 528 (M+H)⁺.

### Step 2. tert-Butyl (S)-4-(7-(4-chloropyridin-2-yl)-5-morpholino-7H-pyrrolo[2,3-d]pyrimidin-4-yl)-3-methylpiperazine-1-carboxylate

To a solution of tert-butyl (S)-4-(7-(4-chloropyridin-2-yl)-5-(3-oxomorpholino)-7H-pyrrolo[2,3-d]pyrimidin-4-yl)-3-methylpiperazine-1-carboxylate (30 mg, 0.057 mmol) in THE (2.0 mL) was added BH₃-THF (2.0 mL, 1.0M) at 0° C. The mixture was stirred at 0° C. for 1 h. The reaction was then quenched by addition of MeOH (5.0 m1) at 0° C. and was then stirred at 25° C. for 0.5 hr. The mixture was concentrated in vacuo and the residue purified by flash chromatography (silica gel column, petroleum ether:EtOAc=50:1 to 30:1) to afford tert-butyl (S)-4-(7-(4-chloropyridin-2-yl)-5-morpholino-7H-pyrrolo[2,3-d]pyrimidin-4-yl)-3-methylpiperazine-1-carboxylate (20 mg) as a yellow oil. LC/MS ESI (m/z): 514 (M+H)⁺.

### Step 3. tert-Butyl (S)-4-(7-(4-cyanopyridin-2-yl)-5-morpholino-7H-pyrrolo[2,3-d]pyrimidin-4-yl)-3-methylpiperazine-1-carboxylate

To a solution of tert-butyl (S)-4-(7-(4-chloropyridin-2-yl)-5-morpholino-7H-pyrrolo[2,3-d]pyrimidin-4-yl)-3-methylpiperazine-1-carboxylate (50 mg, 0.097 mmol) in DMF (10.0 mL) was added Zn(CN)₂ (230 mg, 2.0 mmol) and Pd(PPh₃)₄ (110 mg, 0.097 mmol) at 25° C. The mixture was degassed under N₂ three times and stirred at 120° C. for 12 h. The mixture was then cooled to rt and poured into H₂O (10 mL) and extracted with EtOAc twice. The combined organic phases were washed with brine, dried over Na₂SO₄, filtered, and concentrated in vacuo. The crude product was purified by Prep-HPLC to afford tert-butyl (S)-4-(7-(4-cyanopyridin-2-yl)-5-morpholino-7H-pyrrolo[2,3-d]pyrimidin-4-yl)-3-methylpiperazine-1-carboxylate (3.0 mg, 6.0%) as a yellow solid. LC/MS ESI (m/z): 505 (M+H)⁺. ¹H NMR (400 MHz, DMSO-d6) δ 9.14 (s, 1H), 8.73 (d, J=5.0 Hz, 1H), 8.45 (s, 1H), 7.71 (dd, J=5.1, 1.3 Hz, 1H), 7.64 (s, 1H), 5.01 (s, 1H), 4.20 (d, J=14.5 Hz, 1H), 4.06-3.91 (m, 2H), 3.80 (M, 5H), 3.07-2.76 (m, 6H), 1.42 (s, 9H), 1.04 (d, 3H).

The following compounds were prepared by an analogous procedure to compound 380 varying the corresponding amides.

| Cmpd No. | Chemical Name | LCMS and ¹H NMR |
|---|---|---|
| 405 | tert-butyl (S)-4-(7-(4- | LC/MS ESI (m/z): 517(M + H)⁺. ¹H NMR (400 MHz, |
| | cyanopyridin-2-yl)-5-(3,3- | CDCl₃) δ 9.35 (s, 1H), 8.57 (d, J = 5.0 Hz, 1H), 8.43 (s, |
| | dimethylpyrrolidin-1-yl)-7H- | 1H), 7.52 (s, 1H), 7.29 (d, J = 5.0 Hz, 1H), 5.16-4.92 (m, |
| | pyrrolo[2,3-d]pyrimidin-4-yl)-3- | 1H), 4.32-3.86 (m, 3H), 3.53-3.28 (m, 3H), 3.02-2.81 |
| | methylpiperazine-1-carboxylate | (m, 4H), 1.76 (t, J = 7.1 Hz, 2H), 1.57 (s, 3H), 1.49 (s, 9H), |
| | | 1.20 (d, J = 8.9 Hz, 6H) |
| 381 | tert-butyl (S)-4-(7-(4- | LC/MS ESI (m/z): 518(M + H)⁺ ¹H NMR(400 MHz, |
| | cyanopyridin-2-yl)-5-(4- | CD₃OD) δ 9.24 (s, 1H), 8.66 (d, J = 5.0 Hz, 1H), 8.43 (s, |
| | methylpiperazin-1-yl)-7H- | 1H), 7.77 (s, 1H), 7.51 (dd, J = 5.0, 1.3 Hz, 1H), 5.13- |
| | pyrrolo[2,3-d]pyrimidin-4-yl)-3- | 5.04 (m, 1H), 4.25-4.05 (m, 2H), 3.93 (d, J = 13.1 Hz, |
| | methylpiperazine-1-carboxylate | 1H), 3.60-3.34 (m, 3H), 2.97 (m, J = 99.1, 55.5 Hz, 8H), |
| | | 2.44 (s, 3H), 1.50 (s, 9H), 1.14 (d, J = 6.4 Hz, 3H). |
| 370 | tert-butyl (S)-4-(7-(4- | LC/MS ESI (m/z): 489(M + H)⁺ ¹H NMR(400 MHz, MeOD) |
| | cyanopyridin-2-yl)-5- | δ 9.25 (s, 1H), 8.64 (d, J = 4.7 Hz, 1H), 8.40 (s, 1H), 7.66 |
| | (pyrrolidin-1-yl)-7H- | (s, 1H), 7.48 (dd, J = 5.0, 1.3 Hz, 1H), 5.05 (m, 1H), 4.12 |
| | pyrrolo[2,3-d]pyrimidin-4-yl)-3- | (m, J = 14.5 Hz, 2H), 3.93 (d, J = 13.2 Hz, 1H), 3.39 (m, J = |
| | methylpiperazine-1-carboxylate | 12.9, 9.6 Hz, 1H), 3.23 (m, 3H), 2.95 (m, J = 5.5 Hz, 3H), |
| | | 2.01 (m, 4H), 1.49 (s, 9H), 1.10 (d, J = 6.5 Hz, 3H). |
| 417 | tert-butyl (S)-4-(7-(4- | LC/MS ESI (m/z): 519 (M + H)⁺ ¹H NMR (400 MHz, |
| | cyanopyridin-2-yl)-5-((R)-2- | CDCl₃) δ 9.30 (s, 1H), 8.52 (d, J = 4.8 Hz, 1H), 8.39 (s, |
| | methylmorpholino)-7H- | 1H), 7.57 (s, 1H), 7.25 (d, J = 5.0 Hz, 1H), 4.98 (s, 1H), |
| | pyrrolo[2,3-d]pyrimidin-4-yl)-3- | 4.29-4.04 (m, 2H), 3.93-3.69 (m, 4H), 3.43-3.30 (m, |
| | methylpiperazine-1-carboxylate | 2H), 3.28-3.11 (m, 2H), 2.80 (m, 1H), 2.55-2.33 (m, |
| | | 2H), 1.43 (s, 9H), 1.10-1.05 (m, 3H), 0.83-0.76 (m, 3H). |

### Example 97. Synthesis of tert-butyl (3S)-4-(5-cyclopropyl-7-(3,3-difluorocyclohexyl)-7H-pyrrolo[2,3-d]pyrimidin-4-yl)-3-methylpiperazine-1-carboxylate (Compound 382)

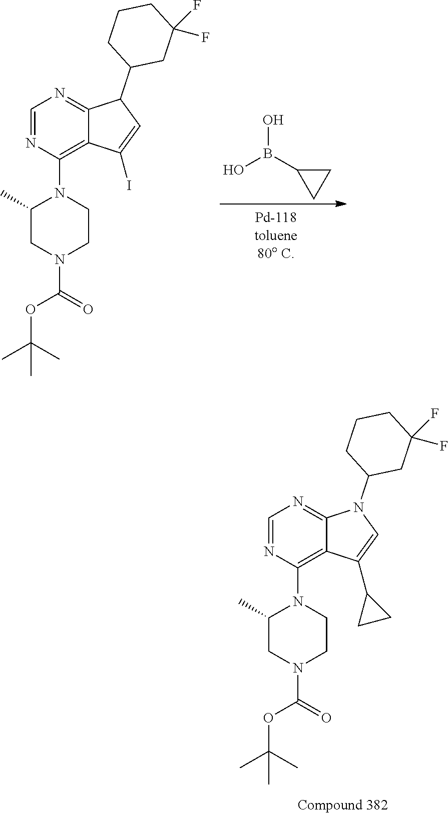 [SMILES: C[C@H]1CN(C(=O)OC(C)(C)C)CCN1c1ncnc2c1C(I)=CC2C1CCCC(F)(F)C1 ; OB(O)C1CC1 ; C[C@H]1CN(C(=O)OC(C)(C)C)CCN1c1ncnc2c1c(C1CC1)cn2C1CCCC(F)(F)C1]

To a solution of tert-butyl (3S)-4-[7-(3,3-difluorocyclohexyl)-5-iodo-7H-pyrrolo[2,3-d]pyrimidin-4-yl]-3-methylpiperazine-1-carboxylate (50 mg, 0.090 mmol, prepared following the procedure outlined in compound 401) in toluene (5 mL) was added cyclopropylboronic acid (7.7 mg, 0.090 mmol), 1,1′-Bis (di-t-butylphosphino)ferrocene palladium dichloride (12 mg, 0.020 mmol) and K₂CO₃ (250 mg, 1.8 mmol). The resulting mixture was heated at 80° C. overnight. After cooling down to room temperature, solvent was removed and the residue was purified by flash column chromatography to afford tert-butyl (3S)-4-(5-cyclopropyl-7-(3,3-difluorocyclohexyl)-7H-pyrrolo[2,3-d]pyrimidin-4-yl)-3-methylpiperazine-1-carboxylate (20 mg, 47%) as a solid. LC/MS ESI (m/z): 476.6 (M+H)⁺. ¹H NMR (400 MHz, CDCl₃) δ 8.38 (s, 1H), 6.65 (s, 1H), 4.91-4.80 (m, 1H), 4.77-4.66 (m, 1H), 4.16-3.76 (m, 3H), 3.60-3.44 (m, 1H), 3.41-2.99 (m, 2H), 2.54-2.42 (m, 1H), 2.35-2.14 (m, 2H), 2.12-1.89 (m, 3H), 1.87-1.69 (m, 3H), 1.49 (s, 9H), 1.25-1.16 (m, 3H), 0.99-0.92 (m, 2H), 0.78-0.58 (m, 2H).

### Example 98. Synthesis of tert-butyl (S)-4-(7-(bicyclo[2.2.2]octan-1-yl)-5-(pyrrolidin-1-yl)-7H-pyrrolo[2,3-d]pyrimidin-4-yl)-3-methylpiperazine-1-carboxylate (Compound 383)

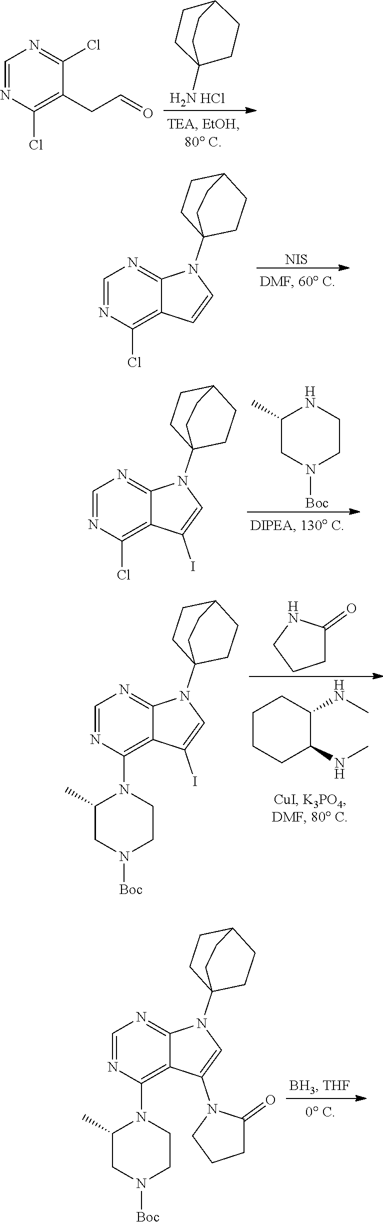

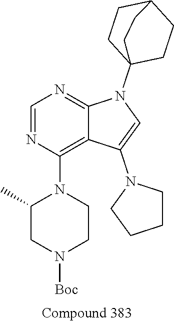 [SMILES: C[C@H]1CN(C(=O)OC(C)(C)C)CCN1c1ncnc2c1c(N1CCCC1)cn2C12CCC(CC1)CC2]

### Step 1. 7-(Bicyclo[2.2.2]octan-1-yl)-4-chloro-7H-pyrrolo[2,3-d]pyrimidine

To a solution of 2-(4,6-dichloropyrimidin-5-yl)acetaldehyde (340 mg, 1.8 mmol) in EtOH (20 mL) was added bicyclo[2.2.2]octan-1-amine hydrochloride (240 mg, 1.5 mmol) and TEA (610 mg, 0.83 mL, 6.0 mmol). The resulting mixture was stirred at 80° C. under N₂ atmosphere for 2 h. The solvent was removed, the residue extracted with EtOAc twice, the combined organic layers were dried over Na₂SO₄, filtered and concentrated. The residue was purified by flash chromatography (silica gel, 0˜10% EtOAc in petroleum ether) to afford 7-(bicyclo[2.2.2]octan-1-yl)-4-chloro-7H-pyrrolo[2,3-d]pyrimidine (330 mg, 84% yield) as a white solid. LC/MS ESI (m/z): 262 (M+H)⁺.

### Step 2. 7-(Bicyclo[2.2.2]octan-1-yl)-4-chloro-5-iodo-7H-pyrrolo[2,3-d]pyrimidine

To a solution of 7-(bicyclo[2.2.2]octan-1-yl)-4-chloro-7H-pyrrolo[2,3-d]pyrimidine (300 mg, 1.1 mmol) in DMF (2 mL) was added NIS (380 mg, 1.7 mmol) and the resulting mixture was heated at 60° C. for 5 h. After cooling down to room temperature, the reaction was partitioned between EtOAc and water, organic layer was separated, aqueous layer was extracted with EtOAc twice, the combined organic layers dried over Na₂SO₄, filtered and concentrated. The residue was purified by flash chromatography (silica gel, 0˜10% EtOAc in petroleum ether) to afford 7-(bicyclo[2.2.2]octan-1-yl)-4-chloro-5-iodo-7H-pyrrolo[2,3-d]pyrimidine (420 mg, 95% yield) as a pink solid. LC/MS ESI (m/z): 388 (M+H)⁺.

### Step 3. tert-Butyl (S)-4-(7-(bicyclo[2.2.2]octan-1-yl)-5-iodo-7H-pyrrolo[2,3-d]pyrimidin-4-yl)-3-methylpiperazine-1-carboxylate

To the solution of 7-(bicyclo[2.2.2]octan-1-yl)-4-chloro-5-iodo-7H-pyrrolo[2,3-d]pyrimidine (360 mg, 1.0 mmol) in DIEA (2 mL) was added tert-butyl (S)-3-methylpiperazine-1-carboxylate (800 mg, 4.0 mmol). The resulting reaction mixture was stirred at 130° C. under N₂ for 5 h. After cooling down to room temperature, the reaction mixture was concentrated under reduced pressure. The residue was extracted with EtOAc twice, the combined organic layers were dried over Na₂SO₄, filtered and concentrated and purified by flash column chromatography (silica gel, 0-30%, ethyl acetate in petroleum ether) to afford tert-butyl (S)-4-(7-(bicyclo[2.2.2]octan-1-yl)-5-iodo-7H-pyrrolo[2,3-d]pyrimidin-4-yl)-3-methylpiperazine-1-carboxylate (390 mg, 71%) as white solid. LC/MS ESI (m/z): 552 (M+H)⁺.

### Step 4. tert-Butyl (S)-4-(7-(bicyclo[2.2.2]octan-1-yl)-5-(2-oxopyrrolidin-1-yl)-7H-pyrrolo[2,3-d]pyrimidin-4-yl)-3-methylpiperazine-1-carboxylate

To a solution of tert-butyl (S)-4-(7-(bicyclo[2.2.2]octan-1-yl)-5-iodo-7H-pyrrolo[2,3-d]pyrimidin-4-yl)-3-methylpiperazine-1-carboxylate (140 mg, 0.25 mmol) in DMF (5 mL) was added CuI (47 mg, 0.25 mmol), trans-N,N′-dimethylcyclohexane-1,2-diamine (28 mg, 0.25 mmol), K₃CO₄ (420 mg, 2.0 mmol) and pyrrolidin-2-one (170 mg, 2.0 mmol) and the reaction was stirred at 80° C. under N₂ overnight. After cooling down to room temperature, the mixture was extracted with EtOAc twice. The combined organic layers were dried over Na₂SO₄, filtered, and concentrated under reduced pressure. The residue was purified by flash column chromatography (silica gel, 30˜100%, ethyl acetate in petroleum ether) to afford tert-butyl (S)-4-(7-(bicyclo[2.2.2]octan-1-yl)-5-(2-oxopyrrolidin-1-yl)-7H-pyrrolo[2,3-d]pyrimidin-4-yl)-3-methylpiperazine-1-carboxylate (74 mg, 58%) as white solid. LC/MS ESI (m/z): 509 (M+H)⁺.

### Step 5. tert-Butyl (S)-4-(7-(bicyclo[2.2.2]octan-1-yl)-5-(pyrrolidin-1-yl)-7H-pyrrolo[2,3-d]pyrimidin-4-yl)-3-methylpiperazine-1-carboxylate

To the solution of tert-butyl (S)-4-(7-(bicyclo[2.2.2]octan-1-yl)-5-(2-oxopyrrolidin-1-yl)-7H-pyrrolo[2,3-d]pyrimidin-4-yl)-3-methylpiperazine-1-carboxylate (74 mg, 0.15 mmol) in THE (2 mL) maintained in an ice bath was added BH₃/THF (2.9 mL, 2.9 mmol, 1.0 M). The reaction mixture was stirred at 0° C. under N₂ for 2 h. The reaction mixture was quenched by MeOH while in the ice bath, and extracted with EtOAc twice. The combined organic layers were dried over Na₂SO₄, filtered, and concentrated under reduced pressure. The residue was purified by Prep-TLC (silica gel, 20%, ethyl acetate in petroleum ether) to afford tert-butyl (S)-4-(7-(bicyclo[2.2.2]octan-1-yl)-5-(pyrrolidin-1-yl)-7H-pyrrolo[2,3-d]pyrimidin-4-yl)-3-methylpiperazine-1-carboxylate (28 mg, 39%) as withe solid. LC/MS ESI (m/z): 495 (M+H)⁺. ¹H NMR (400 MHz, CDCl₃) δ 8.30 (s, 1H), 6.57 (s, 1H), 4.90 (s, 1H), 4.07 (d, J=44.4 Hz, 2H), 3.82 (s, 1H), 3.35 (t, J=11.2 Hz, 1H), 3.30-3.17 (m, 1H), 3.17-3.04 (m, 2H), 3.04-2.87 (m, 1H), 2.87-2.71 (m, 2H), 2.38-2.23 (m, 6H), 1.97-1.86 (m, 4H), 1.86-1.75 (m, 6H), 1.72-1.67 (m, 1H), 1.48 (s, 9H), 1.16-1.02 (m, 3H).

### Example 99. Synthesis of 1,1,1-trifluoro-2-methylpropan-2-yl 4-(7-(4-cyanopyridin-2-yl)-5-cyclopropyl-7H-pyrrolo[2,3-d]pyrimidin-4-yl)piperazine-1-carboxylate (Compound 385)

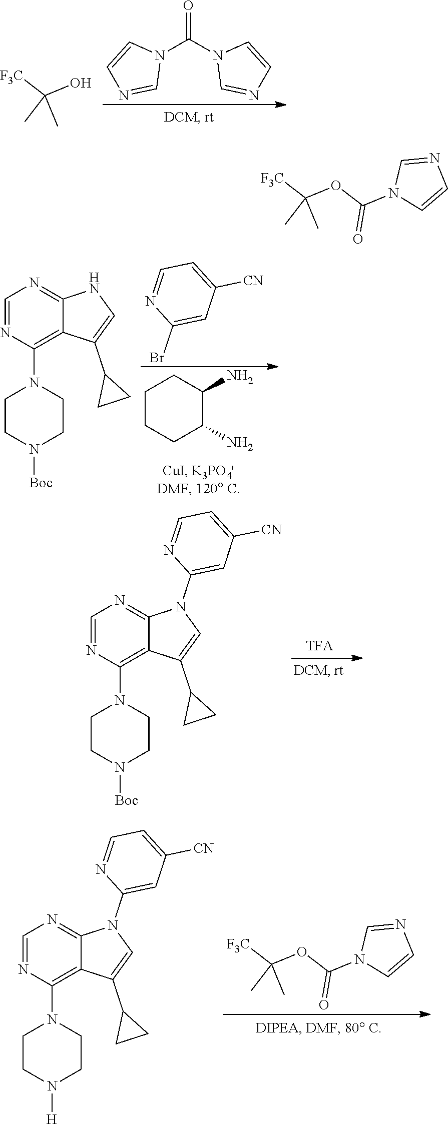

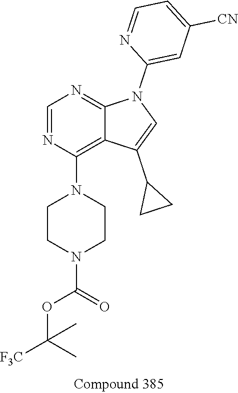 [SMILES: CC(C)(OC(=O)N1CCN(c2ncnc3c2c(C2CC2)cn3-c2cc(C#N)ccn2)CC1)C(F)(F)F]

### Step 1. 1,1,1-Trifluoro-2-methylpropan-2-yl 1H-imidazole-1-carboxylate

To a solution of 1-(1H-imidazole-1-carbonyl)-1H-imidazole (540 mg, 3.4 mmol) in DCM (20 mL) was added 1,1,1-trifluoro-2-methylpropan-2-ol (430 mg, 3.4 mmol) at room temperature, and then the resulting mixture was stirred for 18 h. The mixture was used directly in next step without further purification. LC/MS (ESI) (m/z): 223 (M+H)⁺

### Step 2. tert-Butyl-4-(7-(4-cyanopyridin-2-yl)-5-cyclopropyl-7H-pyrrolo[2,3-d]pyrimidin-4-yl)piperazine-1-carboxylate

A suspension of tert-butyl-4-{5-cyclopropyl-7H-pyrrolo[2,3-d]pyrimidin-4-yl}piperazine-1-carboxylate (240 mg, 0.70 mmol, prepared following the procedure outlined in compound 146, 2-bromopyridine-4-carbonitrile (150 mg, 0.84 mmol), K₃PO₄ (300 mg, 1.4 mmol), CuI (40 mg, 0.21 mmol) and trans-cyclohexane-1,2-diamine (24 mg, 0.21 mmol) in DMF (5 mL) was stirred at 120° C. under N₂ atmosphere for 18 h, and then the mixture was cooled to room temperature and concentrated. The residue was purified by column chromatography on silica gel (0˜10% MeOH in DCM) to give tert-butyl-4-[7-(4-cyanopyridin-2-yl)-5-cyclopropyl-7H-pyrrolo[2,3-d]pyrimidin-4-yl]piperazine-1-carboxylate (280 mg, 90%) as a white solid. LC/MS(ESI) m/z: 446 (M+H)⁺.

### Step 3. 2-(5-Cyclopropyl-4-(piperazin-1-yl)-7H-pyrrolo[2,3-d]pyrimidin-7-yl)isonicotinonitrile

To a 0° C. solution of tert-butyl-4-[7-(4-cyanopyridin-2-yl)-5-cyclopropyl-7H-pyrrolo[2,3-d]pyrimidin-4-yl]piperazine-1-carboxylate (280 mg, 0.63 mmol) in DCM (2 mL) was added TFA (4.5 mL, 60 mmol). The resulting mixture was stirred at 0° C. for 2 h. The mixture was diluted with DCM, washed with NaHCO₃ (aq.), the organic layer was extracted with DCM twice, the combined organic layers were washed with brine, dried over Na₂SO₄, filtered and concentrated. The residue was used directly in the next step. LC/MS ESI (m/z): 346 (M+H)⁺.

### Step 4. 1,1,1-Trifluoro-2-methylpropan-2-yl 4-(7-(4-cyanopyridin-2-yl)-5-cyclopropyl-7H-pyrrolo[2,3-d]pyrimidin-4-yl)piperazine-1-carboxylate

To a solution of 2-[5-cyclopropyl-4-(piperazin-1-yl)-7H-pyrrolo[2,3-d]pyrimidin-7-yl]pyridine-4-carbonitrile (100 mg, 0.29 mmol) and DIEA (0.19 mL, 1.2 mmol) in DMF (5 mL) was added a solution of 1,1,1-trifluoro-2-methylpropan-2-yl-1H-imidazole-1-carboxylate (96 mg, 0.43 mmol), and the resulting mixture was stirred at rt for 18 h. After removal of solvent, the residue was added partitioned between DCM and water, the organic layer separated, and the aqueous layer was extracted with DCM twice. The combined organic phases were washed with brine, dried over anhydrous Na₂SO₄, filtered and concentrated. The residue was purified by column chromatography on silica gel and prep-HPLC to give 1,1,1-trifluoro-2-methylpropan-2-yl-4-[7-(4-cyanopyridin-2-yl)-5-cyclopropyl-7H-pyrrolo[2,3-d]pyrimidin-4-yl]piperazine-1-carboxylate (20 mg, 14%) as a white solid. LC/MS (ESI) (m/z): 500 (M+H)^{+ 1}H NMR (400 MHz, CDCl₃) δ 9.32 (s, 1H), 8.58 (d, J=5.0 Hz, 1H), 8.52 (s, 1H), 7.80 (s, 1H), 7.35 (dd, J=5.0, 1.2 Hz, 1H), 3.71 (s, 4H), 3.64 (s, 4H), 2.04 (ddd, J=12.0, 7.6, 4.8 Hz, 1H), 1.73 (s, 6H), 1.09-1.01 (m, 2H), 0.88-0.79 (m, 2H).

### Example 100. Synthesis of isopropyl (S)-4-(7-(4-cyanopyridin-2-yl)-5-cyclopropyl-7H-pyrrolo[2,3-d]pyrimidin-4-yl)-3-methylpiperazine-1-carboxylate (Compound 386)

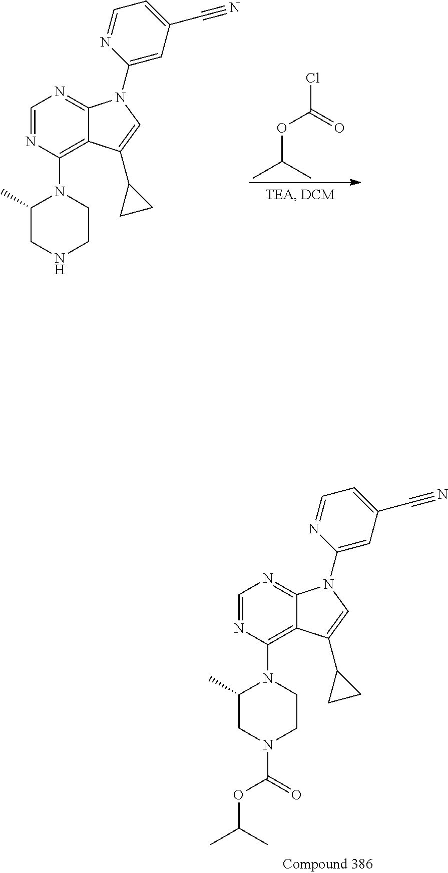

Isopropyl (S)-4-(7-(4-Cyanopyridin-2-yl)-5-cyclopropyl-7H-pyrrolo[2,3-d]pyrimidin-4-yl)-3-methylpiperazine-1-carboxylate

To a 0° C. solution of (S)-2-(5-cyclopropyl-4-(2-methylpiperazin-1-yl)-7H-pyrrolo[2,3-d]pyrimidin-7-yl)isonicotinonitrile (50 mg, 0.14 mmol, prepared following the procedure outlined in compound 368) in DCM (3 mL) was added TEA (0.050 mL, 0.42 mmol) and isopropyl carbonochloridate (26 mg, 0.21 mmol) dropwise. The resulting mixture was stirred at rt for 20 min under N₂. The reaction was quenched with NaHCO₃ (aq.), extracted with DCM twice, the combined organic layers were washed with brine, dried over Na₂SO₄, filtered and concentrated. The residue was purified by flash column chromatography (silica gel, 0-50%, ethyl acetate in petroleum ether) to give crude product which was further purified by prep-HPLC to afford isopropyl (S)-4-(7-(4-cyanopyridin-2-yl)-5-cyclopropyl-7H-pyrrolo[2,3-d]pyrimidin-4-yl)-3-methylpiperazine-1-carboxylate (20 mg, 32%) as a white solid. LC/MS ESI (m/z): 446 (M+H)⁺. ¹H NMR (400 MHz, CD₃OD) δ 9.12 (s, 1H), 8.66 (dd, J=5.0, 0.7 Hz, 1H), 8.47 (s, 1H), 7.87 (d, J=0.9 Hz, 1H), 7.55 (dd, J=5.0, 1.3 Hz, 1H), 4.93 (m, J=12.5, 6.2 Hz, 1H), 4.80 (m, J=7.2, 4.6 Hz, 1H), 4.12 (d, J=13.3 Hz, 1H), 3.93 (m, J=13.2 Hz, 2H), 3.64-3.56 (m, 1H), 3.42 (m, J=10.5, 5.7 Hz, 1H), 3.27-3.15 (m, 1H), 2.11-2.04 (m, 1H), 1.28 (d, J=6.2 Hz, 6H), 1.23 (d, J=6.6 Hz, 3H), 1.08 (m, J=8.1, 4.1 Hz, 2H), 0.91-0.85 (m, 1H), 0.78 (ddd, J=10.7, 6.5, 4.1 Hz, 1H).

### Example 101. Synthesis of tert-butyl (S)-4-(5-cyclopropyl-7-(4-fluoro-3-(trifluoromethoxy)phenyl)-7H-pyrrolo[2,3-d]pyrimidin-4-yl)-3-methylpiperazine-1-carboxylate (Compound 387)

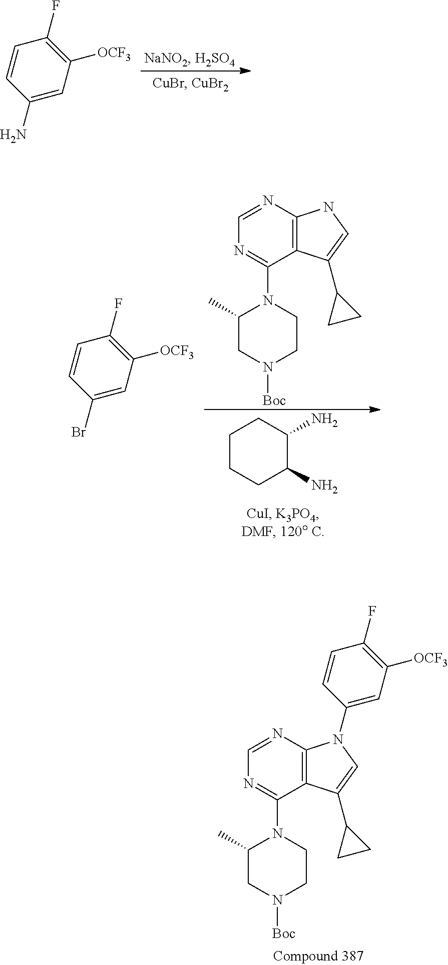

### Step 1. 4-Fluoro-3-(trifluoromethoxy)aniline

A mixture of 4-fluoro-3-(trifluoromethoxy)aniline (200 mg, 1.0 mmol) in H₂SO₄ (5 mL, 30% in H₂O) was cooled to 0° C., and NaNO₂ (140 mg, 2.0 mmol) added carefully. After being stirred at 0° C. for 0.5 h, a solution of CuBr (290 mg, 2.0 mmol) and CuBr₂ (450 g, 2.0 mmol) in HBr (2.5 mL, 48% wt in H₂O) was added dropwise to the reaction mixture maintaining the temperature. The mixture was warmed to room temperature and stirred overnight. The reaction was then diluted with DCM, washed with water and brine, dried over Na₂SO₄ and concentrated in vacuo to give crude 4-bromo-1-fluoro-2-(trifluoromethoxy)benzene (390 mg, DCM seen in NMR), which was used directly in the next step without any further purification. ¹H NMR (400 MHz, CDCl₃) δ 7.47 (d, J=6.8 Hz, 1H), 7.42 (ddd, J=8.8, 4.1, 2.4 Hz, 1H), 7.10 (t, J=9.2 Hz, 1H). ¹⁹F NMR (377 MHz, CDCl₃) δ −58.85 (d, J=5.3 Hz), −130.06 (q, J=4.8 Hz).

### Step 2. tert-Butyl (S)-4-(5-cyclopropyl-7-(4-fluoro-3-(trifluoromethoxy)phenyl)-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (71 mg, 0.20 mmol, prepared similarly as in compound 259, step 4) and 4-bromo-1-fluoro-2-(trifluoromethoxy)benzene (200 mg, 0.30 mmol, contaminated with DCM) in DMF (1 mL) was added CuI (12 mg, 0.060 mmol), K₃PO₄ (130 mg, 0.60 mmol) and trans-1,2-diaminocyclohexane (7.0 mg, 0.060 mmol). The resulting mixture was stirred at 120° C. overnight. After cooling down to room temperature, the reaction was partitioned between EtOAc and water, organic layer was separated, aqueous layer was extracted with EtOAc twice, the combined organic layers were washed with brine, filtered and concentrated. The residue was purified by flash column chromatography (silica gel, 0˜30%, ethyl acetate in petroleum ether) to give crude product which was further purified by prep-HPLC to afford tert-butyl (S)-4-(5-cyclopropyl-7-(4-fluoro-3-(trifluoromethoxy)phenyl)-7H-pyrrolo[2,3-d]pyrimidin-4-yl)-3-methylpiperazine-1-carboxylate (5.1 mg, 4.7% yield) as a white solid. LC/MS ESI (m/z): 536 (M+H)⁺. ¹H NMR (400 MHz, CDCl₃) δ 8.42 (s, 1H), 7.74-7.54 (m, 2H), 7.33 (t, J=9.1 Hz, 1H), 6.87 (s, 1H), 4.88-4.62 (m, 1H), 4.24-3.75 (m, 3H), 3.64-3.47 (m, 1H), 3.44-3.25 (m, 1H), 3.24-3.00 (m, 1H), 2.06-1.99 (m, 1H), 1.50 (s, 9H), 1.24 (d, J=6.6 Hz, 3H), 1.07-0.98 (m, 2H), 0.84-0.75 (m, 1H), 0.75-0.66 (m, 1H).

### Example 102. Synthesis of tert-butyl (R)-4-(7-(4-cyanopyridin-2-yl)-5-(2-fluorophenyl)-7H-pyrrolo[2,3-d]pyrimidin-4-yl)-3-methylpiperazine-1-carboxylate (Compound 389)

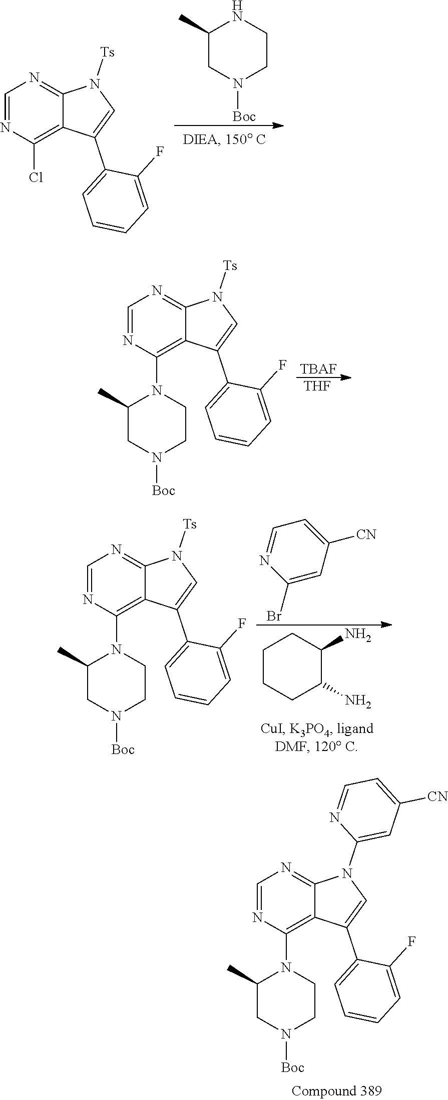

### Step 1. tert-Butyl (R)-4-(5-(2-fluorophenyl)-7-tosyl-7H-pyrrolo[2,3-d]pyrimidin-4-yl)-3-methylpiperazine-1-carboxylate

A mixture of 4-chloro-5-(2-fluorophenyl)-7-tosyl-7H-pyrrolo[2,3-d]pyrimidine (600 mg, 1.50 mmol, prepared following the procedure outlined for compound 392) and tert-butyl (R)-3-methylpiperazine-1-carboxylate (600 mg, 3.0 mmol) in DIEA (20 mL) was heated at 150° C. for 3 hours. The reaction mixture was concentrated and the residue was purified by column chromatography on silica gel (0˜30% EtOAc in petroleum ether, V/V) to give the tert-butyl (R)-4-(5-(2-fluorophenyl)-7-tosyl-7H-pyrrolo[2,3-d]pyrimidin-4-yl)-3-methylpiperazine-1-carboxylate (520 mg, 62%) as a yellow solid. LC/MS ESI (m/z): 566 (M+H)⁺.

### Step 2. tert-Butyl (R)-4-(5-(2-fluorophenyl)-7H-pyrrolo[2,3-d]pyrimidin-4-yl)-3-methylpiperazine-1-carboxylate

To a solution of tert-butyl (R)-4-(5-(2-fluorophenyl)-7-tosyl-7H-pyrrolo[2,3-d]pyrimidin-4-yl)-3-methylpiperazine-1-carboxylate (500 mg, 0.88 mmol) in THE (10 mL) was added TBAF (5.0 mL, 1.0M solution in THF) and the resulting mixture was stirred at room temperature for 12 hrs. The reaction mixture was then extracted with EtOAc (100 m1×2). The combined layers were washed with brine, dried over Na₂SO₄ and concentrated. The residue was purified by column chromatography on silica gel (0˜80% EtOAc in petroleum ether) to give the tert-butyl (R)-4-(5-(2-fluorophenyl)-7H-pyrrolo[2,3-d]pyrimidin-4-yl)-3-methylpiperazine-1-carboxylate (100 mg, 27%) as a yellow solid. LC/MS ESI (m/z): 412 (M+H)⁺.

### Step 3. tert-Butyl (R)-4-(7-(4-cyanopyridin-2-yl)-5-(2-fluorophenyl)-7H-pyrrolo[2,3-d]pyrimidin-4-yl)-3-methylpiperazine-1-carboxylate

A mixture of tert-butyl (R)-4-(5-(2-fluorophenyl)-7H-pyrrolo[2,3-d]pyrimidin-4-yl)-3-methylpiperazine-1-carboxylate (100 mg, 0.24 mmol), 2-bromopyridine-4-carbonitrile (89 mg, 0.49 mmol), CuI (23 mg, 0.12 mmol), trans-1,2-diaminocyclohexane (8.3 mg, 0.070 mmol) and K₃PO₄ (150 mg, 0.73 mmol) in DMF (10 mL) was heated at 120° C. for 12 hours. The reaction mixture was concentrated and the residue was purified by column chromatography (silica gel, 0˜40% EtOAc in petroleum ether) to afford tert-butyl (R)-4-(7-(4-cyanopyridin-2-yl)-5-(2-fluorophenyl)-7H-pyrrolo[2,3-d]pyrimidin-4-yl)-3-methylpiperazine-1-carboxylate (80 mg, 64%) as a white solid. LC/MS ESI (m/z): 514 (M+H)⁺. ¹H NMR (400 MHz, CD₃OD) δ 9.30 (s, 1H), 8.70 (d, J=5.0 Hz, 1H), 8.55 (s, 1H), 8.29 (s, 1H), 7.61 (dd, J=5.0, 1.3 Hz, 1H), 7.54 (td, J=7.5, 1.7 Hz, 1H), 7.50-7.43 (m, 1H), 7.34 (dd, J=7.5, 6.5 Hz, 1H), 7.31-7.24 (m, 1H), 4.19-4.11 (m, 1H), 3.72 (d, J=13.6 Hz, 1H), 3.49 (d, J=11.7 Hz, 2H), 3.13-3.06 (m, 1H), 2.73 (m, 2H), 1.42 (s, 9H), 0.97 (d, J=6.5 Hz, 3H).

The following compound was prepared by a procedure analogous to the synthesis of compound 389 using the corresponding amine.

| Cmpd No. | Chemical Name | LCMS and ¹H NMR |
|---|---|---|
| 390 | tert-butyl (2R,5S)-4-(7-(4- | LC/MS ESI (m/z): 528.2 (M + H)⁺. ¹H NMR (400 MHz, |
| | cyanopyridin-2-yl)-5-(2- | CDCl3) δ 9.37-9.33 (m, 1H), 8.55 (d, J = 4.9 Hz, 1H), |
| | fluorophenyl)-7H-pyrrolo[2,3- | 8.50 (s, 1H), 8.22 (s, 1H), 7.46-7.38 (m, 1H), 7.34-7.29 |
| | d]pyrimidin-4-yl)-2,5- | (m, 2H), 7.15 (dd, J = 17.2, 7.8 Hz, 2H), 4.13 (m, 2H), 3.27 |
| | dimethylpiperazine-1- | (t, J = 13.6 Hz, 2H), 3.20-3.12 (m, 1H), 2.80 (m, 1H), 1.36 |
| | carboxylate | (s, 9H), 0.99 (d, J = 6.8 Hz, 3H), 0.90 (d, J = 6.6 Hz, 3H). |

### Example 103. Synthesis of 2-(5-(2-fluorophenyl)-4-(4-isobutyryl-3,3-dimethylpiperazin-1-yl)-7H-pyrrolo[2,3-d]pyrimidin-7-yl)isonicotinonitrile (Compound 391)

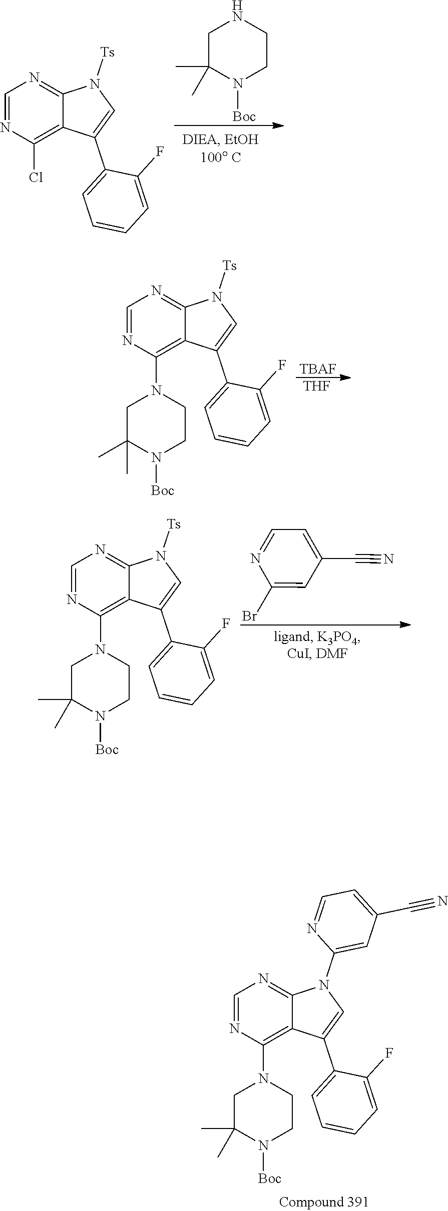

### Step 1. tert-Butyl 4-(5-(2-fluorophenyl)-7-tosyl-7H-pyrrolo[2,3-d]pyrimidin-4-yl)-2,2-dimethylpiperazine-1-carboxylate

To a solution of 4-chloro-5-cyclopropyl-7-tosyl-7H-pyrrolo[2,3-d]pyrimidine (600 mg, 1.7 mmol) in EtOH (10 mL) was added tert-butyl 2,2-dimethylpiperazine-1-carboxylate (410 mg, 1.9 mmol) and DIPEA (450 mg, 3.5 mmol) and the resulting mixture was heated at 100° C. for 8 hours. After cooling down to room temperature, the reaction was quenched with water, extracted with EtOAc twice, the combined organic layers were washed with brine, dried over Na₂SO₄, filtered and concentrated. The residue was purified by flash column chromatography (silica gel, 0-100%, ethyl acetate in petroleum ether) to afford tert-butyl 4-(5-(2-fluorophenyl)-7-tosyl-7H-pyrrolo[2,3-d]pyrimidin-4-yl)-2,2-dimethylpiperazine-1-carboxylate (800 mg, 79%) as a white solid. LC/MS ESI (m/z): 580 (M+H)⁺.

### Step 2. tert-Butyl 4-(5-(2-fluorophenyl)-7H-pyrrolo[2,3-d]pyrimidin-4-yl)-2,2-dimethylpiperazine-1-carboxylate

To a solution of tert-butyl 4-(5-(2-fluorophenyl)-7-tosyl-7H-pyrrolo[2,3-d]pyrimidin-4-yl)-2,2-dimethylpiperazine-1-carboxylate (800 mg, 1.4 mmol) in THE (5 mL) was added TBAF (10 mL) and the resulting mixture stirred at room temperature for 4 h. The reaction was quenched with water, extracted with EtOAc twice, the combined organic layers were washed with brine, dried over Na₂SO₄, filtered and concentrated. The residue was purified by flash column chromatography (silica gel, 0˜100%, ethyl acetate in petroleum ether) to afford tert-butyl 4-(5-(2-fluorophenyl)-7H-pyrrolo[2,3-d]pyrimidin-4-yl)-2,2-dimethylpiperazine-1-carboxylate (400 mg, 68%) as a white solid. LC/MS ESI (m/z): 426 (M+H)⁺.

### Step 3. tert-Butyl (S)-3-methyl-4-(5-(pyridin-2-yl)-7-tosyl-7H-pyrrolo[2,3-d]pyrimidin-4-yl)piperazine-1-carboxylate

To a solution of tert-butyl 4-(5-(2-fluorophenyl)-7H-pyrrolo[2,3-d]pyrimidin-4-yl)-2,2-dimethylpiperazine-1-carboxylate (280 mg, 0.70 mmol) in DMF (10 mL) was added 2-bromoisonicotinonitrile (240 mg, 1.3 mmol), trans-cyclohexane-1,2-diamine (47 mg, 0.30 mmol), CuI (25 mg, 0.10 mmol) and K₃PO₄ (200 mg, 2.0 mmol) and the resulting mixture was heated at 110° C. overnight. After cooling down to room temperature, the reaction was quenched with water, extracted with EtOAc twice, the combined organic layers were washed with brine, dried over Na₂SO₄, filtered and concentrated. The residue was purified by flash column chromatography (silica gel, 0-100%, ethyl acetate in petroleum ether) to give crude product which was further purified by prep-HPLC to afford tert-butyl 4-(7-(4-cyanopyridin-2-yl)-5-(2-fluorophenyl)-7H-pyrrolo[2,3-d]pyrimidin-4-yl)-2,2-ethylpiperazine-1-carboxylate (7.3 mg, 2.0%) as a white solid. LC/MS ESI (m/z): 528 (M+H)⁺. ¹H NMR (400 MHz, MeOD) δ 9.42 (s, 1H), 8.62 (d, J=5.0 Hz, 1H), 8.52 (s, 1H), 8.20 (s, 1H), 7.45 (td, J=7.5, 1.7 Hz, 1H), 7.41-7.36 (m, 2H), 7.25-7.21 (m, 1H), 7.18 (t, J=9.1 Hz, 1H), 3.50-3.42 (m, 4H), 3.26 (s, 2H), 1.44 (s, 9H), 1.22 (s, 6H).

The following compound was prepared by the procedure analogous to the synthesis of compound 391 from the corresponding aryl halide.

| Cmpd | | |
|---|---|---|
| No. | Chemical Name | LCMS and ¹H NMR |
| 395 | tert-butyl 4-(7-(4-cyanopyridin- | LC/MS ESI (m/z): 474 (M + H)+. ¹H NMR (400 MHz, |
| | 2-yl)-5-cyclopropyl-7H- | CDCl₃) δ 9.33 (s, 1H), 8.58 (d, J = 5.0 Hz, 1H), 8.45 (s, |
| | pyrrolo[2,3-d]pyrimidin-4-yl)- | 1H), 7.81 (d, J = 0.8 Hz, 1H), 7.34 (dd, J = 5.0, 1.2 Hz, |
| | 2,2-dimethylpiperazine-1- | 1H), 3.98-3.93 (m, 2H), 3.86 (m, 2H), 3.85-3.82 (m, |
| | carboxylate | 2H), 1.98-1.89 (m, 1H), 1.51 (s, 9H), 1.45 (s, 6H), 1.06- |
| | | 1.01 (m, 2H), 0.84-0.79 (m, 2H). |

### Example 104. Synthesis of tert-butyl (S)-4-(7-(5-cyanothiazol-2-yl)-5-cyclopropyl-7H-pyrrolo[2,3-d]pyrimidin-4-yl)-3-methylpiperazine-1-carboxylate (Compound 397)

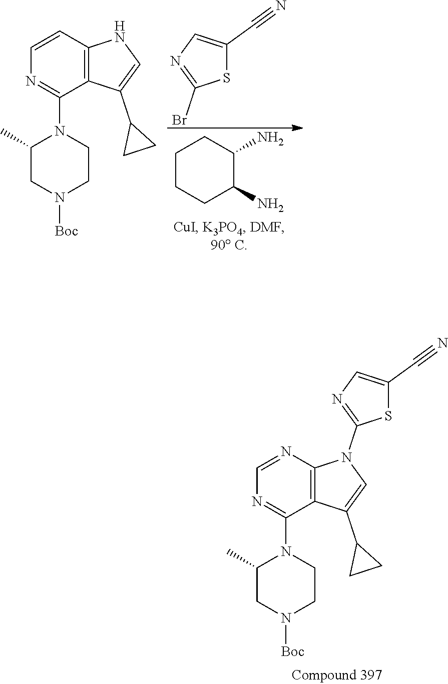

### Step 1. tert-Butyl (S)-4-(7-(5-cyanothiazol-2-yl)-5-cyclopropyl-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (71 mg, 0.20 mmol, prepared following the procedure of compound 260, step 4) and 2-bromothiazole-5-carbonitrile (76 mg, 0.40 mmol) in DMF (1 mL) was added CuI (12 mg, 0.060 mmol), K₃PO₄ (130 mg, 0.60 mmol) and trans-1,2-diaminocyclohexane (7.0 mg, 0.060 mmol) and the resulting mixture stirred at 90° C. overnight. After cooling down to room temperature, the reaction was partitioned between EtOAc and brine, the organic layer separated, aqueous layer extracted with EtOAc twice, the combined organic layers washed with brine, dried over Na₂SO₄, filtered and concentrated. The residue was purified by flash column chromatography (silica gel, 0˜30%, ethyl acetate in petroleum ether) to give crude product which was further purified by prep-HPLC to afford tert-butyl (S)-4-(7-(5-cyanothiazol-2-yl)-5-cyclopropyl-7H-pyrrolo[2,3-d]pyrimidin-4-yl)-3-methylpiperazine-1-carboxylate (14 mg, 15% yield) as a white solid. LC/MS ESI (m/z): 466 (M+H)⁺. ¹H NMR (400 MHz, CDCl₃) δ 8.52 (s, 1H), 8.03 (s, 1H), 7.60 (s, 1H), 5.00-4.60 (m, 1H), 4.33-3.99 (m, 2H), 3.99-3.77 (m, 1H), 3.54 (t, J=11.7 Hz, 1H), 3.38-3.17 (m, 1H), 3.17-2.92 (m, 1H), 2.00-1.86 (m, 1H), 1.50 (s, 9H), 1.27 (d, J=6.6 Hz, 3H), 1.11-1.00 (m, 2H), 0.89-0.77 (m, 2H).

### Example 105. Synthesis of tert-butyl (S)-4-(7-cyclobutyl-5-cyclopropyl-7H-pyrrolo[2,3-d]pyrimidin-4-yl)-3-methylpiperazine-1-carboxylate (Compound 398)

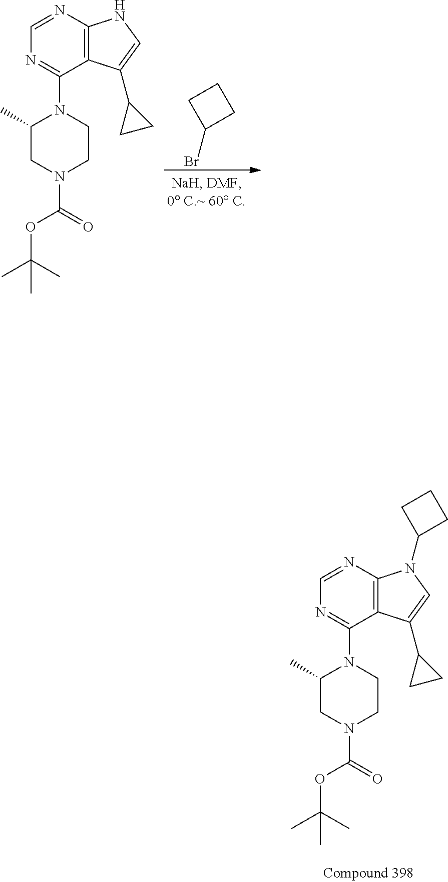

### Step 1. tert-Butyl (S)-4-(7-cyclobutyl-5-cyclopropyl-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (180 mg, 0.50 mmol, prepared following the procedure of compound 260, step 4) in DMF (1 mL) was added NaH (40 mg, 1.0 mmol, 60% wt. in mineral oil) in an ice bath. The resulting mixture was stirred at 0° C. under N₂ atmosphere for 20 min. A solution of bromocyclobutane (140 mg, 1.0 mmol) in DMF (1 mL) was then added to the reaction mixture. The reaction mixture was stirred at 60° C. under N₂ atmosphere overnight. After cooling down to room temperature, the reaction was quenched with NH₄Cl aqueous solution, extracted with EtOAc, the organic layer separated, aqueous layer was extracted with EtOAc twice, the combined organic layers washed with brine, dried over Na₂SO₄, filtered and concentrated. The residue was purified by flash column chromatography (silica gel, 0˜40%, ethyl acetate in petroleum ether) to afford tert-butyl (S)-4-(7-cyclobutyl-5-cyclopropyl-7H-pyrrolo[2,3-d]pyrimidin-4-yl)-3-methylpiperazine-1-carboxylate (66 mg, 32% yield) as a white solid. LC/MS ESI (m/z): 412 (M+H)⁺. ¹H NMR (400 MHz, CDCl₃) δ 8.37 (s, 1H), 6.85 (s, 1H), 5.33-5.17 (m, 1H), 4.88-4.56 (m, 1H), 4.17-3.72 (m, 3H), 3.50 (t, J=10.9 Hz, 1H), 3.40-3.22 (m, 1H), 3.21-3.00 (m, 1H), 2.55-2.44 (m, 2H), 2.44-2.30 (m, 2H), 2.04-1.94 (m, 1H), 1.94-1.80 (m, 2H), 1.49 (s, 9H), 1.19 (d, J=6.5 Hz, 3H), 1.01-0.91 (m, 2H), 0.81-0.71 (m, 1H), 0.70-0.61 (m, 1H).

### Example 106. Synthesis of tert-butyl (S)-4-(7-(3-chloro-5-cyanophenyl)-5-(pyrrolidin-1-yl)-7H-pyrrolo[2,3-d]pyrimidin-4-yl)-3-methylpiperazine-1-carboxylate (Compound 399)

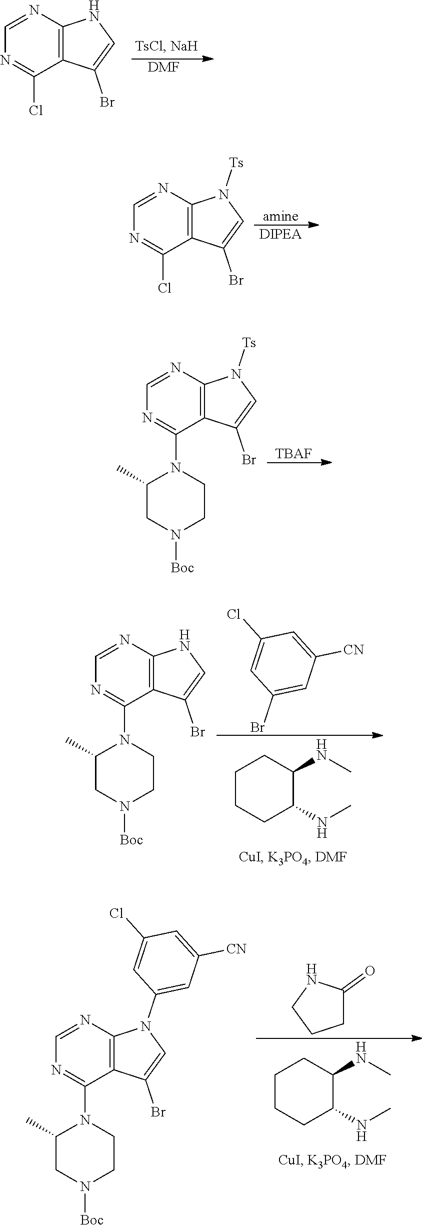

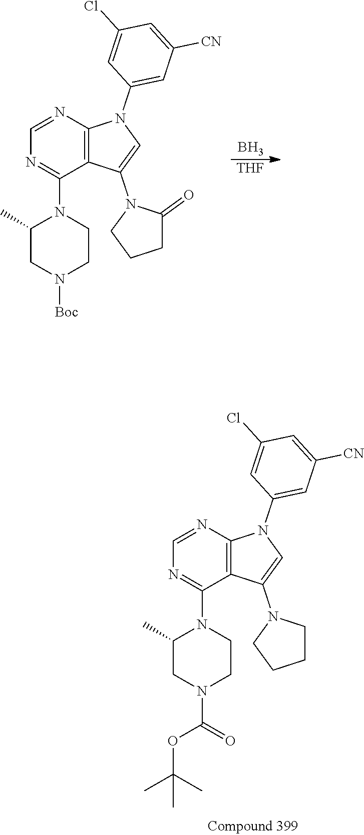

### Step 1. 5-Bromo-4-chloro-7-tosyl-7H-pyrrolo[2,3-d]pyrimidine

To a 0° C. suspension of NaH (1.0 g, 26 mmol, 60% wt. in mineral oil) in anhydrous DMF (25 mL) was added 5-bromo-4-chloro-7H-pyrrolo[2,3-d]pyrimidine (5.0 g, 22 mmol) in portions, the resulting mixture stirred at 0° C. for additional 20 minutes after which, 4-methylbenzenesulfonyl chloride (4.9 g, 26 mmol) was added in portions. The reaction was stirred at room temperature overnight. The reaction was poured into ice water, and the precipitate was collected by filtration. 5-bromo-4-chloro-7-tosyl-7H-pyrrolo[2,3-d]pyrimidine (5.4 g, 65%) as a white solid. LC/MS ESI (m/z): 386, 388 (M+H)⁺.

### Step 2. tert-Butyl (S)-4-(5-bromo-7-tosyl-7H-pyrrolo[2,3-d]pyrimidin-4-yl)-3-methylpiperazine-1-carboxylate

To a solution of 5-bromo-4-chloro-7-(4-methylbenzenesulfonyl)-7H-pyrrolo[2,3-d]pyrimidine (3.0 g, 7.8 mmol) in DIPEA (9.5 mL, 55 mmol) was added tert-butyl (S)-3-methylpiperazine-1-carboxylate (6.2 mg, 31 mmol). The resulting mixture was heated at 150° C. for 3 h under N₂. After cooling down to room temperature, the solvent was removed and the residue was purified by flash column chromatography to afford tert-butyl (S)-4-(5-bromo-7-tosyl-7H-pyrrolo[2,3-d]pyrimidin-4-yl)-3-methylpiperazine-1-carboxylate (3.5 g, 83%) as a solid. LC/MS ESI (m/z): 550, 552 (M+H)⁺.

### Step 2. tert-Butyl (S)-4-(5-bromo-7H-pyrrolo[2,3-d]pyrimidin-4-yl)-3-methylpiperazine-1-carboxylate

To a solution of tert-butyl (S)-4-(5-bromo-7-tosyl-7H-pyrrolo[2,3-d]pyrimidin-4-yl)-3-methylpiperazine-1-carboxylate (3.0 g, 5.4 mmol) in THE (20 mL) was added TBAF (20 mL, 1.0M in THF). The resulting mixture was stirred at room temperature overnight. The reaction was quenched with water, extracted with EtOAc twice, the combined organic layers washed with brine, dried over Na₂SO₄, filtered and concentrated. The residue was purified by flash column chromatography to afford tert-butyl (S)-4-(5-bromo-7H-pyrrolo[2,3-d]pyrimidin-4-yl)-3-methylpiperazine-1-carboxylate (500 mg, 23%) as a solid. LC/MS ESI (m/z): 396, 398 (M+H)⁺.

### Step 3. tert-Butyl (S)-4-(5-bromo-7-(3-chloro-5-cyanophenyl)-7H-pyrrolo[2,3-d]pyrimidin-4-yl)-3-methylpiperazine-1-carboxylate

To a solution of tert-butyl (S)-4-(5-bromo-7H-pyrrolo[2,3-d]pyrimidin-4-yl)-3-methylpiperazine-1-carboxylate (300 mg, 0.78 mmol) in DMF (10 mL) was added 3-bromo-5-chlorobenzonitrile (160 mg, 0.78 mmol), trans-N,N-dimethyl-cyclohexane-1,2-diamine (210 mg, 1.5 mmol), CuI (270 mg, 1.4 mmol) and K₃PO₄ (320 mg, 1.5 mmol) and the resulting mixture was heated at 90° C. overnight. After cooling down to rt, the reaction was partitioned between EtOAc and water, organic layer was separated, aqueous layer extracted with EtOAc twice, the combined organic layers washed with brine, dried over Na₂SO₄, filtered and concentrated. The residue was purified by flash column chromatography to afford tert-butyl (S)-4-(5-bromo-7-(3-chloro-5-cyanophenyl)-7H-pyrrolo[2,3-d]pyrimidin-4-yl)-3-methylpiperazine-1-carboxylate (350 mg, 87%) as a solid. LC/MS ESI (m/z): 531, 533 (M+H)⁺.

### Step 4. tert-Butyl (S)-4-(7-(3-chloro-5-cyanophenyl)-5-(2-oxopyrrolidin-1-yl)-7H-pyrrolo[2,3-d]pyrimidin-4-yl)-3-methylpiperazine-1-carboxylate

To a solution of tert-butyl (S)-4-(5-bromo-7-(3-chloro-5-cyanophenyl)-7H-pyrrolo[2,3-d]pyrimidin-4-yl)-3-methylpiperazine-1-carboxylate (300 mg, 0.56 mmol) in DMF (10 mL) was added pyrrolidin-2-one (0.17 mL, 2.3 mmol), trans-N,N-dimethyl-cyclohexane-1,2-diamine (0.18 mL, 1.1 mmol), CuI (130 mg, 0.70 mmol) and K₃PO₄ (240 mg, 1.1 mmol) and the resulting mixture was heated to 90° C. overnight. After cooling down to room temperature, the reaction was partitioned between EtOAc and water, organic layer separated, aqueous layer extracted with EtOAc twice, the combined organic layers washed with brine, dried over Na₂SO₄, filtered and concentrated. The residue was purified by flash column chromatography to afford tert-butyl (S)-4-(7-(3-chloro-5-cyanophenyl)-5-(2-oxopyrrolidin-1-yl)-7H-pyrrolo[2,3-d]pyrimidin-4-yl)-3-methylpiperazine-1-carboxylate (130 mg, 43%) as a solid. LC/MS ESI (m/z): 536 (M+H)⁺.

### Step 5. tert-Butyl (S)-4-(7-(3-chloro-5-cyanophenyl)-5-(pyrrolidin-1-yl)-7H-pyrrolo[2,3-d]pyrimidin-4-yl)-3-methylpiperazine-1-carboxylate

To a 0° C. solution of tert-butyl (S)-4-(7-(3-chloro-5-cyanophenyl)-5-(2-oxopyrrolidin-1-yl)-7H-pyrrolo[2,3-d]pyrimidin-4-yl)-3-methylpiperazine-1-carboxylate (60 mg, 0.11 mmol) in THE (4 mL) was added borane (2.0 mL, 1.0M in THF) dropwise. The resulting mixture was warmed to 25° C. over 3 h. The reaction was quenched with MeOH after cooling in an ice water bath, and then diluted with ethyl acetate and water. The organic layers were separated, the aqueous layer extracted with EtOAc twice, the combined organic layers washed with brine, dried over anhydrous sodium sulfate and concentrated. The residue was purified by flash column chromatography to afford tert-butyl (S)-4-(7-(3-chloro-5-cyanophenyl)-5-(pyrrolidin-1-yl)-7H-pyrrolo[2,3-d]pyrimidin-4-yl)-3-methylpiperazine-1-carboxylate (10 mg, 17%) as a yellow solid. LC/MS ESI (m/z): 522 (M+H)⁺. ¹H NMR (400 MHz, CDCl₃) δ 8.38 (s, 1H), 8.14-8.09 (m, 2H), 7.53-7.48 (m, 1H), 6.68 (s, 1H), 5.05-4.88 (m, 1H), 4.25-3.87 (m, 3H), 3.44-3.13 (m, 4H), 3.02-2.81 (m, 3H), 1.99 (s, 4H), 1.49 (s, 9H), 1.18-1.08 (m, 3H).

### Example 107. Synthesis of tert-butyl (3S)-4-(7-(3,3-difluorocyclohexyl)-5-(pyrrolidin-1-yl)-7H-pyrrolo[2,3-d]pyrimidin-4-yl)-3-methylpiperazine-1-carboxylate (Compound 401)

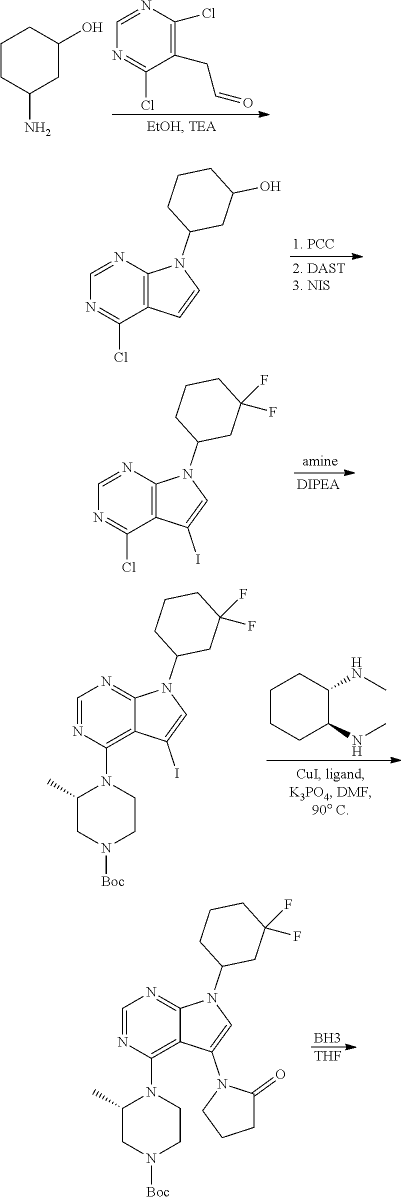

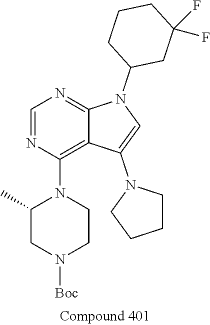 [SMILES: C[C@H]1CN(C(=O)OC(C)(C)C)CCN1c1ncnc2c1c(N1CCCC1)cn2C1CCCC(F)(F)C1]

### Step 1. 3-(4-Chloro-7H-pyrrolo[2,3-d]pyrimidin-7-yl)cyclohexan-1-ol

To a solution of 3-aminocyclohexan-1-ol (1.0 g, 8.7 mmol) in EtOH (10 mL) were added 2-(4,6-dichloropyrimidin-5-yl)acetaldehyde (1.6 g, 8.7 mmol) and TEA (2.9 mL, 21 mmol). The resulting mixture was heated at 80° C. for 3 h under N₂. After cooling down to room temperature, the solvent was removed and the residue was purified by flash column chromatography to afford 3-(4-chloro-7H-pyrrolo[2,3-d]pyrimidin-7-yl)cyclohexan-1-ol (1.5 g, 69%) as a solid. LC/MS ESI (m/z): 252.4 (M+H)⁺.

### Step 2. 3-(4-Chloro-7H-pyrrolo[2,3-d]pyrimidin-7-yl)cyclohexan-1-one

To a solution of 3-(4-chloro-7H-pyrrolo[2,3-d]pyrimidin-7-yl)cyclohexan-1-ol (2.0 g, 7.9 mmol) in DCM (20 mL) was added PCC (2.6 g, 12 mmol). The resulting mixture was stirred at room temperature overnight. The solvent was removed, and the residue was purified by flash column chromatography to afford 3-(4-chloro-7H-pyrrolo[2,3-d]pyrimidin-7-yl)cyclohexan-1-one (1.4 g, 70%) as a solid. LC/MS ESI (m/z): 250.4 (M+H)⁺.

### Step 3. 4-Chloro-7-(3,3-difluorocyclohexyl)-7H-pyrrolo[2,3-d]pyrimidine

To a solution of 3-(4-chloro-7H-pyrrolo[2,3-d]pyrimidin-7-yl)cyclohexan-1-one (1.4 g, 5.6 mmol) in DCM (20 mL) was added DAST (1.4 g, 8.4 mmol) at 0° C. The resulting mixture was allowed to warm to room temperature overnight. The solvent was removed, and the residue was purified by flash column chromatography to afford 4-chloro-7-(3,3-difluorocyclohexyl)-7H-pyrrolo[2,3-d]pyrimidine (1.3 g, 85%) as a solid. LC/MS ESI (m/z): 272.4 (M+H)⁺.

### Step 4. 4-Chloro-7-(3,3-difluorocyclohexyl)-5-iodo-7H-pyrrolo[2,3-d]pyrimidine

To a solution of 4-chloro-7-(3,3-difluorocyclohexyl)-7H-pyrrolo[2,3-d]pyrimidine (600 mg, 2.2 mmol) in DMF (6 mL) was added iodo(sulfanylidene)amine (460 mg, 2.6 mmol). The resulting mixture was heated to 55° C. overnight. After cooling down to room temperature, the solvent was removed and the residue purified by flash column chromatography to afford 4-chloro-7-(3,3-difluorocyclohexyl)-5-iodo-7H-pyrrolo[2,3-d]pyrimidine (350 mg, 40%) as a solid. LC/MS ESI (m/z): 398.3 (M+H)⁺.

### Step 5. tert-Butyl (3S)-4-(7-(3,3-difluorocyclohexyl)-5-iodo-7H-pyrrolo[2,3-d]pyrimidin-4-yl)-3-methylpiperazine-1-carboxylate

To a solution of 4-chloro-7-(3,3-difluorocyclohexyl)-5-iodo-7H-pyrrolo[2,3-d]pyrimidine (300 mg, 0.76 mmol) in DIPEA (10 mL, 55 mmol) was added tert-butyl (3S)-3-methylpiperazine-1-carboxylate (600 mg, 3.0 mmol). The resulting mixture was heated at 150° C. for 3 h under N₂. After cooling down to room temperature, the solvent was removed and the residue was purified by flash column chromatography to afford tert-butyl (3S)-4-(7-(3,3-difluorocyclohexyl)-5-iodo-7H-pyrrolo[2,3-d]pyrimidin-4-yl)-3-methylpiperazine-1-carboxylate (370 mg, 87%) as a solid. LC/MS ESI (m/z): 562.5 (M+H)⁺.

### Step 6. tert-Butyl (3S)-4-(7-(3,3-difluorocyclohexyl)-5-(2-oxopyrrolidin-1-yl)-7H-pyrrolo[2,3-d]pyrimidin-4-yl)-3-methylpiperazine-1-carboxylate

To a solution of tert-butyl (3S)-4-[7-(3,3-difluorocyclohexyl)-5-iodo-7H-pyrrolo[2,3-d]pyrimidin-4-yl]-3-methylpiperazine-1-carboxylate (250 mg, 0.40 mmol) in DMF (5 mL) were added pyrrolidin-2-one (0.14 mL, 1.8 mmol), trans-N,N-dimethyl-cyclohexane-1,2-diamine (130 mg, 0.90 mmol), CuI (85 mg, 0.40 mmol) and K₃PO₄ (95 mg, 0.40 mmol). The resulting mixture was heated at 90° C. overnight. After cooling down to room temperature, the reaction was partitioned between EtOAc and water, the organic layer separated, the aqueous layer extracted with EtOAc twice, the combined organic layers washed with brine, dried over Na₂SO₄, filtered and concentrated. The residue was purified by flash column chromatography to afford tert-butyl (3S)-4-(7-(3,3-difluorocyclohexyl)-5-(2-oxopyrrolidin-1-yl)-7H-pyrrolo[2,3-d]pyrimidin-4-yl)-3-methylpiperazine-1-carboxylate (90 mg, 39%) as a solid. LC/MS ESI (m/z): 519.7 (M+H)⁺.

### Step 7. tert-Butyl (3S)-4-(7-(3,3-difluorocyclohexyl)-5-(pyrrolidin-1-yl)-7H-pyrrolo[2,3-d]pyrimidin-4-yl)-3-methylpiperazine-1-carboxylate

To a solution of tert-butyl (3S)-4-(7-(3,3-difluorocyclohexyl)-5-(2-oxopyrrolidin-1-yl)-7H-pyrrolo[2,3-d]pyrimidin-4-yl)-3-methylpiperazine-1-carboxylate (50 mg, 0.090 mmol) in THE (4 mL) maintained at 0° C. was added borane (2.0 mL, 1.0M in THF). The resulting mixture was allowed to warm to rt over 3 h. The reaction was then quenched with MeOH while cooling in an ice water bath, and then diluted with ethyl acetate. The organic phase was washed with brine and dried with anhydrous sodium sulfate, concentrated, and the residue was purified by flash column chromatography to afford tert-butyl (3S)-4-(7-(3,3-difluorocyclohexyl)-5-(pyrrolidin-1-yl)-7H-pyrrolo[2,3-d]pyrimidin-4-yl)-3-methylpiperazine-1-carboxylate (15 mg, 31%) as a solid. LC/MS ESI (m/z): 505.3 (M+H)⁺. ¹H NMR (400 MHz, CDCl₃) δ 8.31 (s, 1H), 6.44 (s, 1H), 5.03-4.86 (m, 2H), 4.27-3.82 (m, 3H), 3.37-2.81 (m, 7H), 2.54-2.44 (m, 1H), 2.35-2.15 (m, 2H), 2.12-2.05 (m, 1H), 1.98-1.89 (m, 5H), 1.85-1.72 (m, 3H), 1.49 (s, 9H), 1.16-1.04 (m, 3H).

### Example 108. Synthesis of tert-butyl-(S)-4-(7-((1S,2R,4R)-bicyclo[2.2.1]heptan-2-yl)-5-(pyrrolidin-1-yl)-7H-pyrrolo[2,3-d]pyrimidin-4-yl)-3-methylpiperazine-1-carboxylate (Compound 406)

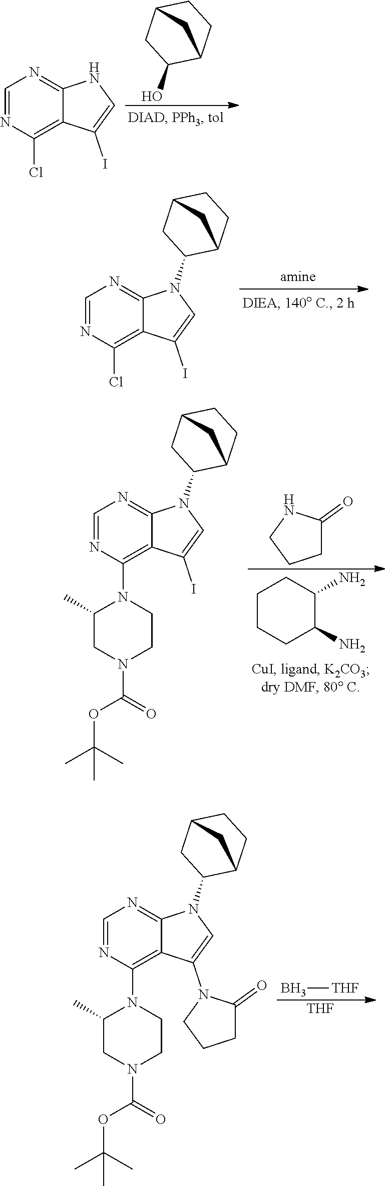

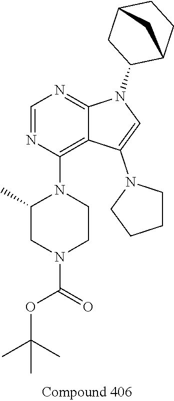 [SMILES: C[C@H]1CN(C(=O)OC(C)(C)C)CCN1c1ncnc2c1c(N1CCCC1)cn2[C@@H]1C[C@@H]2CC[C@H]1C2]

### Step 1. 7-((1S,2R,4R)-Bicyclo[2.2.1]heptan-2-yl)-4-chloro-5-iodo-7H-pyrrolo[2,3-d]pyrimidine

To a 0° C. solution of PPh₃ (4.1 g, 16 mmol) in dry toluene (20 mL) was added DIAD (3.1 mL, 16 mmol) dropwise, followed by 4-chloro-5-iodo-7H-pyrrolo[2,3-d]pyrimidine (2.5 g, 8.9 mmol) in portions. The resulting mixture was stirred at ice bath temperature for 30 minutes. (1S,2S,4R)-Bicyclo[2.2.1]heptan-2-ol (0.50 g, 4.5 mmol) was then added and further stirred for 18 h and allowed to warm to rt. The reaction mixture was quenched by addition of water and EtOAc. The organic layers were separated and the aqueous layer was extracted by EtOAc twice. The combined organic layers were washed with brine, dried over anhydrous Na₂SO₄, filtered and concentrated. The residue was purified by column chromatography on silica gel to get 7-[(1S,2R,4R)-bicyclo[2.2.1]heptan-2-yl]-4-chloro-5-iodo-7H-pyrrolo[2,3-d]pyrimidine (0.20 g, 12%) as a white solid. LC/MS (ESI) (m/z): 374 (M+H)⁺

### Step 2. tert-Butyl-(S)-4-(7-((1S,2R,4R)-bicyclo[2.2.1]heptan-2-yl)-5-iodo-7H-pyrrolo[2,3-d]pyrimidin-4-yl)-3-methylpiperazine-1-carboxylate

To a solution of 7-[(1S,2R,4R)-bicyclo[2.2.1]heptan-2-yl]-4-chloro-5-iodo-7H-pyrrolo[2,3-d]pyrimidine (200 mg, 0.53 mmol) in DIEA (0.35 mL, 2.1 mmol) was added tert-butyl-(3S)-3-methylpiperazine-1-carboxylate (210 mg, 1.1 mmol). The resulting mixture was stirred at 140° C. for 3 h. After removal of solvent, the residue was purified by column chromatography (silica gel, 0˜10% MeOH in DCM) to give tert-butyl-(3S)-4-{7-[(1S,2R,4R)-bicyclo[2.2.1]heptan-2-yl]-5-iodo-7H-pyrrolo[2,3-d]pyrimidin-4-yl}-3-methylpiperazine-1-carboxylate (100 mg, 35%) as a white solid. LC/MS (ESI) (m/z): 538 (M+H)⁺

### Step 3. tert-Butyl-(S)-4-(7-((1S,2R,4R)-bicyclo[2.2.1]heptan-2-yl)-5-(2-oxopyrrolidin-1-yl)-7H-pyrrolo[2,3-d]pyrimidin-4-yl)-3-methylpiperazine-1-carboxylate

A suspension of tert-butyl-(3S)-4-{7-[(1S,2R,4R)-bicyclo[2.2.1]heptan-2-yl]-5-iodo-7H-pyrrolo[2,3-d]pyrimidin-4-yl}-3-methylpiperazine-1-carboxylate (100 mg, 0.18 mmol), pyrrolidin-2-one (0.11 mL, 1.5 mmol), K₃PO₄ (320 mg, 1.5 mmol), CuI (35 mg, 0.18 mmol) and trans-cyclohexane-1,2-diamine (24 mg, 0.21 mmol) in DMF (5 mL) was stirred at 80° C. under N₂ atmosphere for 18 h. The mixture was cooled to room temperature and concentrated. The residue was purified by column chromatography (silica gel, 0˜10% MeOH in DCM) to give tert-butyl-(3S)-4-{7-[(1S,2R,4R)-bicyclo[2.2.1]heptan-2-yl]-5-(2-oxopyrrolidin-1-yl)-7H-pyrrolo[2,3-d]pyrimidin-4-yl}-3-methylpiperazine-1-carboxylate (80 mg, 87%) as a white solid. LC/MS (ESI) (m/z): 495 (M+H)⁺

### Step 4. Trans-N¹-(2-(4-(2-fluorophenyl)piperazin-1-yl)cyclohexyl)-N⁴,N⁴-dimethylbenzene-1,4-disulfonamide

To a 0° C. solution of tert-butyl-(3S)-4-{7-[(1S,2R,4R)-bicyclo[2.2.1]heptan-2-yl]-5-(2-oxopyrrolidin-1-yl)-7H-pyrrolo[2,3-d]pyrimidin-4-yl}-3-methylpiperazine-1-carboxylate (80 mg, 0.16 mmol) in dry THE (3.0 mL) was added BH₃/THF (0.65 mL, 1.0M in THF) dropwise, and the resulting mixture stirred for 18 h. The reaction was quenched by addition of saturated NaHCO₃ and ethyl acetate. The organic layers were separated and the aqueous layer was extracted with EtOAc twice. The combined organic layers were washed with brine, dried over anhydrous Na₂SO₄, filtered and concentrated. The residue was purified by column chromatography (silica gel) and prep-HPLC to give tert-butyl-(S)-4-(7-((1S,2R,4R)-bicyclo[2.2.1]heptan-2-yl)-5-(pyrrolidin-1-yl)-7H-pyrrolo[2,3-d]pyrimidin-4-yl)-3-methylpiperazine-1-carboxylate (13 mg, 17%) as a yellow solid. LC/MS (ESI) (m/z): 481 (M+H)^{+ 1}H NMR (400 MHz, CDCl₃) δ 8.33 (d, J=4.1 Hz, 1H), 6.58 (dd, J=10.7, 4.2 Hz, 1H), 5.14 (dt, J=9.2, 4.4 Hz, 0.8H), 4.99 (dd, J=25.8, 10.5 Hz, 1H), 4.74 (td, J=8.3, 5.0 Hz, 0.2H), 4.32-3.78 (m, 3H), 3.44-2.81 (m, 7H), 2.68 (s, 1H), 2.49-2.36 (m, 1H), 2.25-2.11 (m, 1H), 2.07-1.87 (m, 4H), 1.76-1.57 (m, 4H), 1.52-1.31 (m, 12H), 1.18-1.01 (m, 3H).

### Example 109. Synthesis of tert-butyl (S)-4-(5-(azetidin-1-yl)-7-(4-cyanopyridin-2-yl)-7H-pyrrolo[2,3-d]pyrimidin-4-yl)-3-methylpiperazine-1-carboxylate (Compound 407)

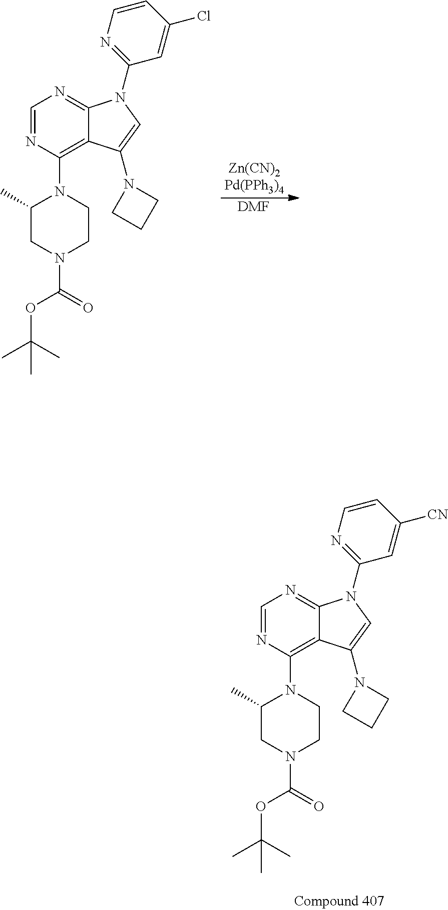

The title compound was prepared by an analogous procedure as described for compound 388, using tert-butyl (S)-4-(5-(azetidin-1-yl)-7-(4-chloropyridin-2-yl)-7H-pyrrolo[2,3-d]pyrimidin-4-yl)-3-methylpiperazine-1-carboxylate as the reactant, as described below.

To Zn(CN)₂ (31 mg, 0.26 mmol) and Pd(PPh₃)₄ (31 mg, 0.027 mmol) was added a solution of tert-butyl (S)-4-(5-(azetidin-1-yl)-7-(4-chloropyridin-2-yl)-7H-pyrrolo[2,3-d]pyrimidin-4-yl)-3-methylpiperazine-1-carboxylate (26 mg, 0.054 mmol) in dry DMF (2 mL). The mixture was stirred at 120° C. under N₂ for 3 h. The reaction was diluted with EtOAc, washed with 5% LiCl (aq.) and brine and concentrated. The residue was purified by prep-TLC (petroleum ether/EtOAc=4:1, v/v) followed by prep-HPLC to provide tert-butyl (S)-4-(5-(azetidin-1-yl)-7-(4-cyanopyridin-2-yl)-7H-pyrrolo[2,3-d]pyrimidin-4-yl)-3-methylpiperazine-1-carboxylate (17 mg, 65%) as a yellow solid. LC/MS ESI (m/z): 475 (M+H)⁺. ¹H NMR (400 MHz, CDCl₃) δ 9.34 (s, 1H), 8.57 (d, J=5.0 Hz, 1H), 8.44 (s, 1H), 7.46 (s, 1H), 7.29 (d, J=5.0 Hz, 1H), 4.96 (br. s, 1H), 4.01 (d, J=82.7 Hz, 3H), 3.82 (dd, J=13.5, 6.6 Hz, 2H), 3.66 (q, J=6.9 Hz, 2H), 3.51 (t, J=11.6 Hz, 1H), 3.40 (br. s, 1H), 2.96 (br. s, 1H), 2.29 (m, J=7.0 Hz, 2H), 1.50 (s, 9H), 1.16 (d, J=6.7 Hz, 3H).

### Example 110. Synthesis of tert-butyl (1S,6R)-5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate (Compound 408 and Compound 409)

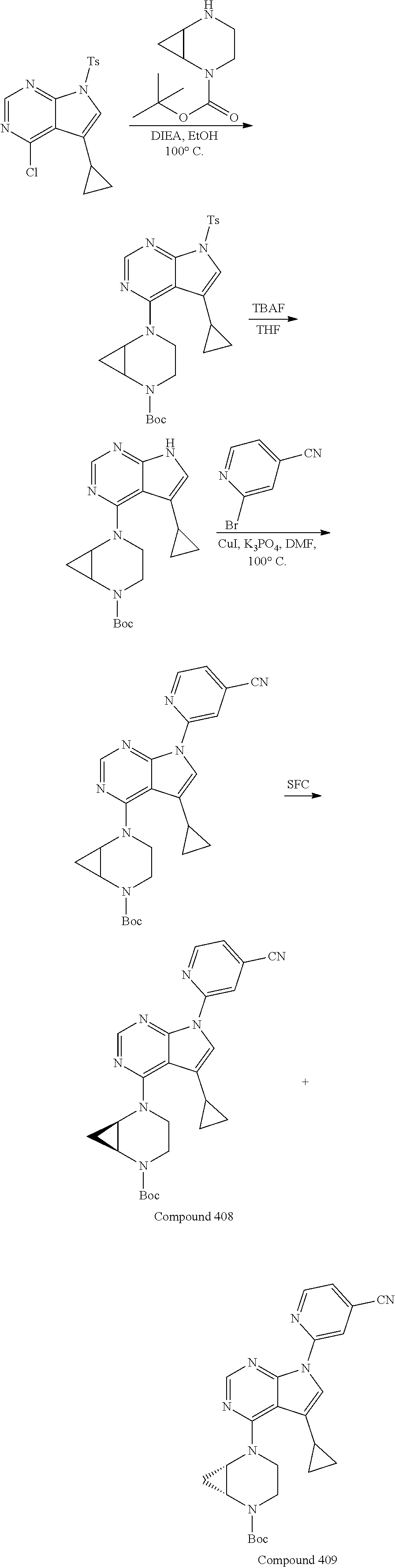

### Step 1. tert-Butyl 5-(5-cyclopropyl-7-tosyl-7H-pyrrolo[2,3-d]pyrimidin-4-yl)-2,5-diazabicyclo[4.1.0]heptane-2-carboxylate

To the solution of 4-chloro-5-cyclopropyl-7-tosyl-7H-pyrrolo[2,3-d]pyrimidine (170 mg, 0.87 mmol) in EtOH (5 mL) were added DIEA (0.14 mL, 0.87 mmol), and tert-butyl 2,5-diazabicyclo[4.1.0]heptane-2-carboxylate (140 mg, 0.72 mmol) in that order. The resulting reaction mixture was stirred at 100° C. under N₂ overnight. After being cooled to room temperature, the solvent was removed, and the residue was purified by flash column chromatography (silica gel, 0˜20%, ethyl acetate in petroleum ether) to afford tert-butyl 5-(5-cyclopropyl-7-tosyl-7H-pyrrolo[2,3-d]pyrimidin-4-yl)-2,5-diazabicyclo[4.1.0]heptane-2-carboxylate (140 mg, 45%) as white solid. LC/MS ESI (m/z): 510 (M+H)⁺.

### Step 2. tert-Butyl 5-(5-cyclopropyl-7H-pyrrolo[2,3-d]pyrimidin-4-yl)-2,5-diazabicyclo[4.1.0]heptane-2-carboxylate

To the solution of tert-butyl 5-(5-cyclopropyl-7-tosyl-7H-pyrrolo[2,3-d]pyrimidin-4-yl)-2,5-diazabicyclo[4.1.0]heptane-2-carboxylate (140 mg, 0.40 mmol) in THE (2 mL) was added TBAF (1.5 mL, 1.0M in THF). The resulting reaction mixture was stirred at rt under N₂ overnight. The reaction mixture was quenched with ice water, extracted with EtOAc twice, the combined organic layers dried over Na₂SO₄, filtered, and concentrated under reduced pressure. The crude product was purified by flash column chromatography (silica gel, 0˜50%, ethyl acetate in petroleum ether) to afford tert-butyl 5-(5-cyclopropyl-7H-pyrrolo[2,3-d]pyrimidin-4-yl)-2,5-diazabicyclo[4.1.0]heptane-2-carboxylate (90 mg, 64%) as a white solid. LC/MS ESI (m/z): 356 (M+H)⁺.

### Step 3. tert-Butyl 5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate

To the solution of tert-butyl 5-(5-cyclopropyl-7H-pyrrolo[2,3-d]pyrimidin-4-yl)-2,5-diazabicyclo[4.1.0]heptane-2-carboxylate (90 mg, 0.25 mmol) in DMF (2 mL) was added CuI (24 mg, 0.13 mmol), K₃PO₄ (160 mg, 0.75 mmol), trans-cyclohexane-1,2-diamine (9.0 mg, 0.080 mmol) and 2-bromoisonicotinonitrile (93 mg, 0.51 mmol) consecutively. The resulting reaction mixture was stirred at 100° C. under N₂ overnight. After being cooled to room temperature, the reaction was diluted with water, and extracted with EtOAc twice. The combined organic layers were dried over Na₂SO₄, filtered, and concentrated under reduced pressure. The crude product was purified by flash column chromatography (silica gel, 0-20%, ethyl acetate in petroleum ether) and prep-HPLC to afford tert-butyl 5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate (66 mg, 57%) as white solid. LC/MS ESI (m/z): 458 (M+H)⁺. Separation of isomers: 44 mg of tert-butyl 5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate was separated by SFC (Preparative separation method, Instrument: Waters Thar 80 preparative SFC, Column: ChiralCel OJ, 250×21.2 mm I.D., 5 μm, Mobile phase: A for CO₂ and B for MEOH+0.1% NH₃/H₂O, Gradient: B 40%, Flow rate: 50 mL/min, Back pressure: 100 bar, Column temperature: 35° C., Wavelength: 254 nm, Cycle-time: 7 min, Elution time: 1.5 h) to give two isomers:

Peak 1: shorter retention time, assigned as compound 408 (tert-butyl (1S,6R)-5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate, 22 mg). LC/MS ESI (m/z): 458 (M+H)⁺. ¹H NMR (400 MHz, CDCl₃) δ 9.33 (d, J=3.2 Hz, 1H), 8.60-8.57 (m, 1H), 8.46-8.39 (m, 1H), 7.85-7.81 (m, 1H), 7.36-7.32 (m, 1H), 4.55-4.46 (m, 1H), 3.84-3.74 (m, 1H), 3.67-3.39 (m, 3H), 3.10-2.98 (m, 1H), 2.07-1.99 (m, 1H), 1.48 (s, 9H), 1.22-1.12 (m, 1H), 1.00-0.74 (m, 4H), 0.50-0.44 (m, 1H).

Peak 2: longer retention time, assigned as compound 409 (tert-butyl (1R,6S)-5-(7-(4-cyanopyridin-2-yl)-5-cyclopropyl-7H-pyrrolo[2,3-d]pyrimidin-4-yl)-2,5-diazabicyclo[4.1.0]heptane-2-carboxylate, 22 mg): LC/MS ESI (m/z): 458 (M+H)⁺. ¹H NMR (400 MHz, CDCl₃) δ 9.37-9.29 (m, 1H), 8.59 (d, J=5.0 Hz, 1H), 8.45-8.40 (m, 1H), 7.85-7.81 (m, 1H), 7.34 (dd, J=5.0, 1.3 Hz, 1H), 4.55-4.46 (m, 1H), 3.84-3.75 (m, 1H), 3.67-3.60 (m, 0.5H), 3.56-3.46 (m, 1H), 3.45-3.36 (m, 1.5H), 3.10-2.98 (m, 1H), 2.07-2.00 (m, 1H), 1.48 (s, 9H), 1.21-1.13 (m, 1H), 0.99-0.90 (m, 2H), 0.82-0.68 (m, 2H), 0.50-0.44 (m, 1H).

### Example 111. Synthesis of tert-butyl (3S)-4-(7-(1-cyanopiperidin-3-yl)-5-cyclopropyl-7H-pyrrolo[2,3-d]pyrimidin-4-yl)-3-methylpiperazine-1-carboxylate (Compound 410)

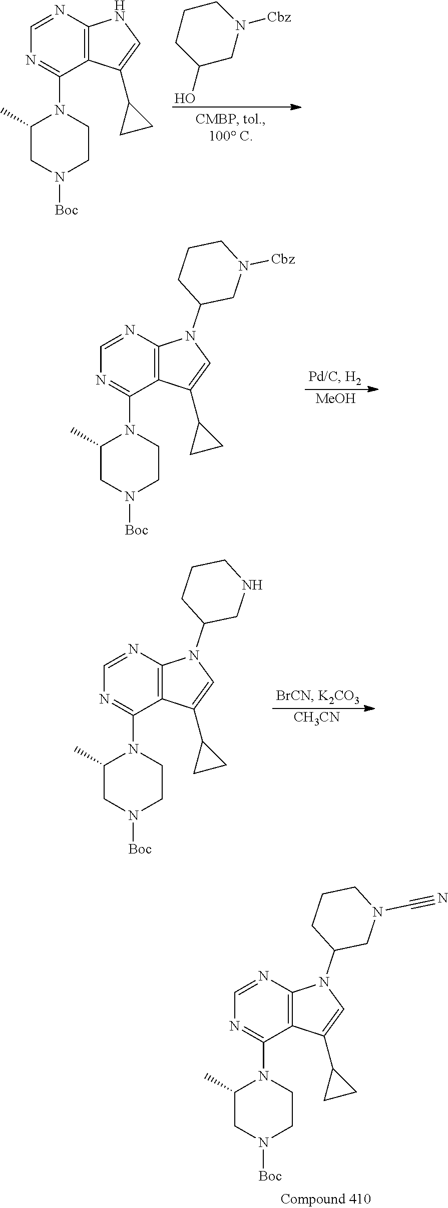

### Step 1. tert-Butyl (3S)-4-(7-(1-((benzyloxy)carbonyl)piperidin-3-yl)-5-cyclopropyl-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (36 mg, 0.10 mmol, prepared following the procedure of compound 260, step 4) and benzyl 3-hydroxypiperidine-1-carboxylate (47 mg, 0.20 mmol) in toluene (1 mL) were added CMBP (48 mg, 0.20 mmol) under N₂ atmosphere. The reaction mixture was stirred at 100° C. overnight under N₂ atmosphere. After cooling to room temperature, the solvent was removed under reduced pressure. the residue was partitioned between EtOAc and water, organic layers separated, aqueous layer extracted with EtOAc twice, the combined organic layers washed with brine, dried over Na₂SO₄, filtered and concentrated. The residue was purified by flash column chromatography (silica gel, 0˜50%, ethyl acetate in petroleum ether) to afford tert-butyl (3S)-4-(7-(1-((benzyloxy)carbonyl)piperidin-3-yl)-5-cyclopropyl-7H-pyrrolo[2,3-d]pyrimidin-4-yl)-3-methylpiperazine-1-carboxylate (35 mg, 60% yield) as a withe solid. LC/MS ESI (m/z): 575 (M+H)⁺.

### Step 2. tert-Butyl (3S)-4-(5-cyclopropyl-7-(piperidin-3-yl)-7H-pyrrolo[2,3-d]pyrimidin-4-yl)-3-methylpiperazine-1-carboxylate

To a solution of tert-butyl (3S)-4-(7-(1-((benzyloxy)carbonyl)piperidin-3-yl)-5-cyclopropyl-7H-pyrrolo[2,3-d]pyrimidin-4-yl)-3-methylpiperazine-1-carboxylate (35 mg, 0.060 mmol) in MeOH (1 mL) was added Pd/C (40 mg, wetted with ca. 55% water) under a N₂ atmosphere. The mixture was stirred at room temperature for 2 h under a H₂ atmosphere. After purging with N₂, the solid was removed by filtration. The solvent was removed under reduced pressure to afford crude tert-butyl (3S)-4-(5-cyclopropyl-7-(piperidin-3-yl)-7H-pyrrolo[2,3-d]pyrimidin-4-yl)-3-methylpiperazine-1-carboxylate as a colorless oil. LC/MS ESI (m/z): 441 (M+H)⁺.

### Step 3. tert-Butyl (3S)-4-(7-(1-cyanopiperidin-3-yl)-5-cyclopropyl-7H-pyrrolo[2,3-d]pyrimidin-4-yl)-3-methylpiperazine-1-carboxylate

To a solution of tert-butyl (3S)-4-(5-cyclopropyl-7-(piperidin-3-yl)-7H-pyrrolo[2,3-d]pyrimidin-4-yl)-3-methylpiperazine-1-carboxylate in CH₃CN (1 mL) were added K₂CO₃ (140 mg, 1.0 mmol) and BrCN (110 mg, 1.0 mmol). The resulting mixture was stirred at room temperature for 1 h under N₂ atmosphere. The reaction was partitioned between EtOAc and water, organic layers separated, aqueous layer extracted with EtOAc twice, the combined organic layers washed with brine, filtered and concentrated. The residue was purified by prep-HPLC to afford tert-butyl (3S)-4-(7-(1-cyanopiperidin-3-yl)-5-cyclopropyl-7H-pyrrolo[2,3-d]pyrimidin-4-yl)-3-methylpiperazine-1-carboxylate (4.2 mg, 15% yield, over two steps) as a white solid. LC/MS ESI (m/z): 466 (M+H)⁺. ¹H NMR (400 MHz, CDCl₃) δ 8.36 (s, 1H), 6.66 (s, 1H), 4.89-4.64 (m, 2H), 4.18-3.73 (m, 3H), 3.64 (dt, J=10.5, 5.2 Hz, 1H), 3.58-3.41 (m, 2H), 3.39-3.20 (m, 2H), 3.19-2.96 (m, 2H), 2.20-2.09 (m, 1H), 2.07-1.98 (m, 1H), 1.97-1.87 (m, 3H), 1.49 (s, 9H), 1.21 (d, J=6.0 Hz, 3H), 1.01-0.91 (m, 2H), 0.77-0.67 (m, 1H), 0.67-0.56 (m, 1H).

### Example 112. Synthesis of tert-butyl 5-(7-(4-cyanopyridin-2-yl)-5-(2-fluorophenyl)-7H-pyrrolo[2,3-d]pyrimidin-4-yl)-2,5-diazabicyclo[4.1.0]heptane-2-carboxylate (Compound

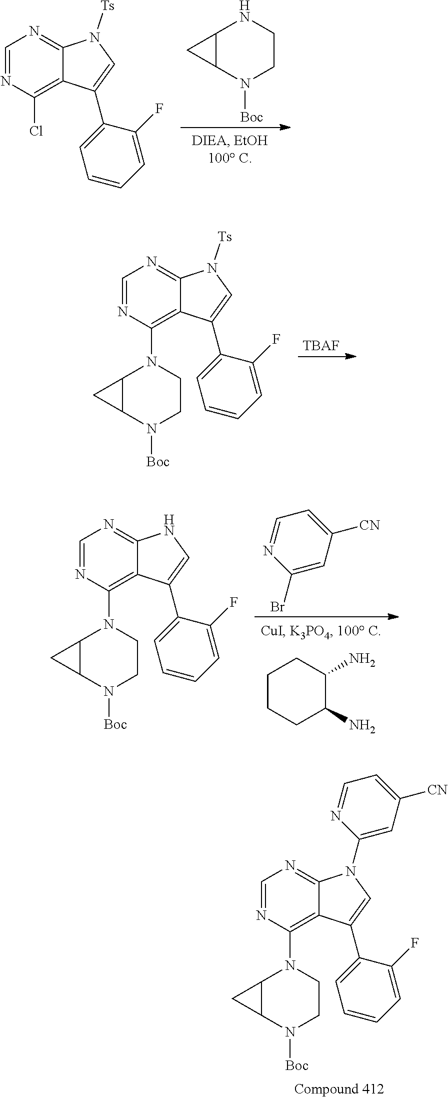

### Step 1. tert-Butyl 5-(5-(2-fluorophenyl)-7-tosyl-7H-pyrrolo[2,3-d]pyrimidin-4-yl)-2,5-diazabicyclo[4.1.0]heptane-2-carboxylate

To the solution of 4-chloro-5-(2-fluorophenyl)-7-tosyl-7H-pyrrolo[2,3-d]pyrimidine (280 mg, 0.70 mmol, Suzuki conditions: Pd₂(dba)₃, xantphos, K₃PO₄, DMF, 60° C.) in EtOH (10 mL) was added DIEA (0.35 mL, 2.1 mmol) and tert-butyl 2,5-diazabicyclo[4.1.0]heptane-2-carboxylate (210 mg, 1.0 mmol) respectively. The resulting reaction mixture was stirred at 100° C. under N₂ overnight. After being cooled down to room temperature, solvent was concentrated under reduced pressure. The crude product was purified by flash column chromatography (silica gel, 0-30%, ethyl acetate in petroleum ether) to afford tert-butyl 5-(5-(2-fluorophenyl)-7-tosyl-7H-pyrrolo[2,3-d]pyrimidin-4-yl)-2,5-diazabicyclo[4.1.0]heptane-2-carboxylate (270 mg, 95%) as white solid. LC/MS ESI (m/z): 564 (M+H)⁺.

### Step 2. tert-Butyl 5-(5-(2-fluorophenyl)-7H-pyrrolo[2,3-d]pyrimidin-4-yl)-2,5-diazabicyclo[4.1.0]heptane-2-carboxylate

To the solution of tert-butyl 5-(5-(2-fluorophenyl)-7-tosyl-7H-pyrrolo[2,3-d]pyrimidin-4-yl)-2,5-diazabicyclo[4.1.0]heptane-2-carboxylate (270 mg, 0.66 mmol) in THE (4 mL) was added TBAF (2.8 mL). The resulting reaction mixture was stirred at rt under N₂ overnight. The reaction was quenched with ice water, then it was extracted with ethyl acetate and the organic layers washed with brine. The combined organic layers were dried over Na₂SO₄, filtered, and concentrated under reduced pressure. The crude product was purified by flash column chromatography (silica gel, 0-50%, ethyl acetate in petroleum ether) to obtain tert-butyl 5-(5-(2-fluorophenyl)-7H-pyrrolo[2,3-d]pyrimidin-4-yl)-2,5-diazabicyclo[4.1.0]heptane-2-carboxylate (160 mg, 57%) as a white solid. LC/MS ESI (m/z): 410 (M+H)⁺.

### Step 3. tert-Butyl 5-(7-(4-cyanopyridin-2-yl)-5-(2-fluorophenyl)-7H-pyrrolo[2,3-d]pyrimidin-4-yl)-2,5-diazabicyclo[4.1.0]heptane-2-carboxylate

To a solution of tert-butyl 5-(5-(2-fluorophenyl)-7H-pyrrolo[2,3-d]pyrimidin-4-yl)-2,5-diazabicyclo[4.1.0]heptane-2-carboxylate (160 mg, 0.38 mmol) in DMF (3 mL) was added CuI (36 mg, 0.19 mmol), K₃PO₄ (240 mg, 1.1 mmol), trans-cyclohexane-1,2-diamine (0.014 mL, 0.11 mmol) and 2-bromoisonicotinonitrile (140 mg, 0.76 mmol) and the resulting reaction mixture was stirred at 100° C. under N₂ overnight. After being cooled down to room temperature, the reaction mixture was poured into ice water, was extracted with ethyl acetate twice. The combined organic layers were dried over Na₂SO₄, filtered, and concentrated under reduced pressure. The crude product was purified by flash column chromatography (silica gel, 0˜20%, ethyl acetate in petroleum ether) to afford tert-butyl 5-(7-(4-cyanopyridin-2-yl)-5-(2-fluorophenyl)-7H-pyrrolo[2,3-d]pyrimidin-4-yl)-2,5-diazabicyclo[4.1.0]heptane-2-carboxylate (170 mg, 88%) as white solid. LC/MS ESI (m/z): 512 (M+H)⁺. ¹H NMR (400 MHz, CDCl₃) δ 9.42 (s, 1H), 8.61 (d, J=5.0 Hz, 1H), 8.48 (s, 1H), 8.18 (s, 1H), 7.48 (t, J=7.5 Hz, 1H), 7.40-7.38 (m, 1H), 7.37-7.31 (m, 1H), 7.22-7.12 (m, 2H), 4.48-4.39 (m, 1H), 3.44-3.28 (m, 1H), 3.28-3.17 (m, 2H), 2.66-2.57 (m, 1H), 2.41-2.33 (m, 1H), 1.43-1.38 (m, 9H), 0.63-0.52 (m, 1H), 0.20-0.13 (m, 1H).

### Example 113. Synthesis of tert-butyl (S)-4-(7-(5-cyano-1-methyl-1H-pyrazol-3-yl)-5-cyclopropyl-7H-pyrrolo[2,3-d]pyrimidin-4-yl)-3-methylpiperazine-1-carboxylate (Compound 413)

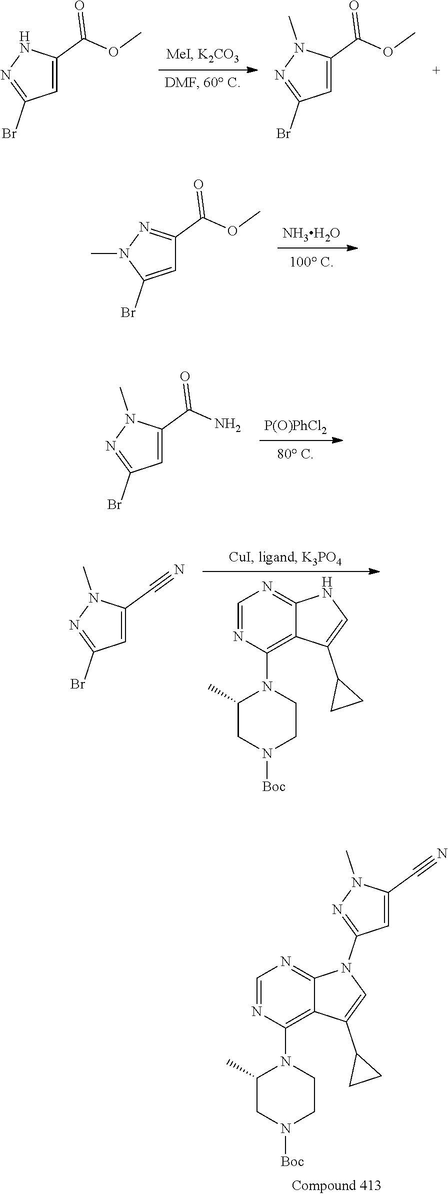

### Step 1. Methyl 3-bromo-1-methyl-1H-pyrazole-5-carboxylate

To a solution of methyl 3-bromo-1H-pyrazole-5-carboxylate (620 mg, 3.0 mmol) in DMF (6 mL) was added K₂CO₃ (620 mg, 4.5 mmol) and MeI (2.1 g, 15 mmol). The mixture was stirred at 60° C. for 2 h. The reaction mixture was diluted with water and extracted with EtOAc. The combined organic layers were washed with brine, dried over Na₂SO₄, filtered, and concentrated under reduced pressure to give a residue. The residue was purified by column chromatography (silica gel, 0-40% ethyl acetate in petroleum ether) and concentrated to afford two compounds:

Methyl 3-bromo-1-methyl-1H-pyrazole-5-carboxylate (420 mg, 63%) as a white solid. LC/MS ESI (m/z): 219/221 (Br) (M+H)⁺. ¹H NMR (400 MHz, CDCl₃) δ 6.81 (s, 1H), 4.15 (s, 3H), 3.88 (s, 3H).

Methyl 5-bromo-1-methyl-1H-pyrazole-3-carboxylate (190 mg, 29%) as a white solid. LC/MS ESI (m/z): 219/221 (Br) (M+H)⁺. ¹H NMR (400 MHz, CDCl₃) δ 6.83 (s, 1H), 3.96 (s, 3H), 3.92 (s, 3H).

### Step 2. 3-Bromo-1-methyl-1H-pyrazole-5-carboxamide

Methyl 3-bromo-1-methyl-1H-pyrazole-5-carboxylate (500 mg, 2.3 mmol) was dissolved in MeOH (2 mL) and NH₃/H₂O (4 mL). The reaction mixture was heated at 100° C. for 2 h. The reaction was cooled to rt, diluted with water and ethyl acetate and layers were separated. The organic layers were dried over Na₂SO₄, filtered and concentrated to afford 3-bromo-1-methyl-1H-pyrazole-5-carboxamide as a white solid (290 mg, 63% yield). LC/MS ESI (m/z): 204/206 (Br) (M+H)⁺.

### Step 3. 3-Bromo-1-methyl-1H-pyrazole-5-carbonitrile

3-Bromo-1-methyl-1H-pyrazole-5-carboxamide (280 mg, 1.4 mmol) was dissolved in PhP(O)Cl₂ (3 mL) and stirred at 80° C. overnight. After cooling in an ice bath, the reaction was quenched with aqueous NaHCO₃, diluted with brine and layers were separated. The organic layers were dried over Na₂SO₄, filtered, concentrated and purified by flash column chromatography (silica gel, 0˜20% ethyl acetate in petroleum ether) to afford 3-bromo-1-methyl-1H-pyrazole-5-carbonitrile as a white solid (230 mg, 89% yield). LC/MS ESI (m/z): none. ¹H NMR (400 MHz, CDCl₃) δ 6.77 (s, 1H), 4.04 (s, 3H).

### Step 5. tert-Butyl (S)-4-(7-(5-cyano-1-methyl-1H-pyrazol-3-yl)-5-cyclopropyl-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (90 mg, 0.25 mmol, prepared following the procedure of compound 260, step 4) and 3-bromo-1-methyl-1H-pyrazole-5-carbonitrile (70 mg, 0.38 mmol) in DMF (1 mL) was added CuI (24 mg, 0.13 mmol), K₃PO₄ (210 mg, 1.0 mmol) and trans-N,N′-dimethylcyclohexane-1,2-diamine (18 mg, 0.13 mmol) and the resulting mixture was stirred at 100° C. overnight under a N₂ atmosphere. After being cooled down to room temperature, the reaction was partitioned between EtOAc and water, organic layers separated, aqueous layer extracted with EtOAc twice, the combined organic layers were washed with brine, dried over Na₂SO₄ filter and concentrated. The residue was purified by flash column chromatography (silica gel, 0˜40%, ethyl acetate in petroleum ether) to give crude product which was further purified by prep-HPLC to afford tert-butyl (S)-4-(7-(5-cyano-1-methyl-1H-pyrazol-3-yl)-5-cyclopropyl-7H-pyrrolo[2,3-d]pyrimidin-4-yl)-3-methylpiperazine-1-carboxylate (13 mg, 11% yield) as a white solid. LC/MS ESI (m/z): 463 (M+H)⁺. ¹H NMR (400 MHz, CDCl₃) δ 8.44 (s, 1H), 7.64 (s, 1H), 7.28 (s, 1H), 4.79-4.66 (m, 1H), 4.34-3.66 (m, 6H), 3.53 (t, J=11.3 Hz, 1H), 3.36-3.03 (m, 2H), 2.06-1.95 (m, 1H), 1.49 (s, 9H), 1.22 (d, J=6.4 Hz, 3H), 1.07-0.93 (m, 2H), 0.87-0.79 (m, 1H), 0.77-0.68 (m, 1H).

### Example 114. Synthesis of tert-butyl (S)-4-(7-(3-cyanocyclobutyl)-5-cyclopropyl-7H-pyrrolo[2,3-d]pyrimidin-4-yl)-3-methylpiperazine-1-carboxylate (Compound 414)

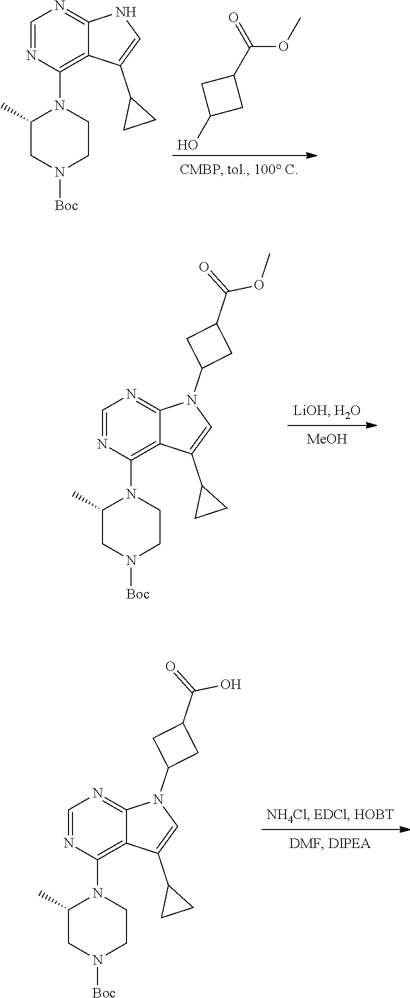

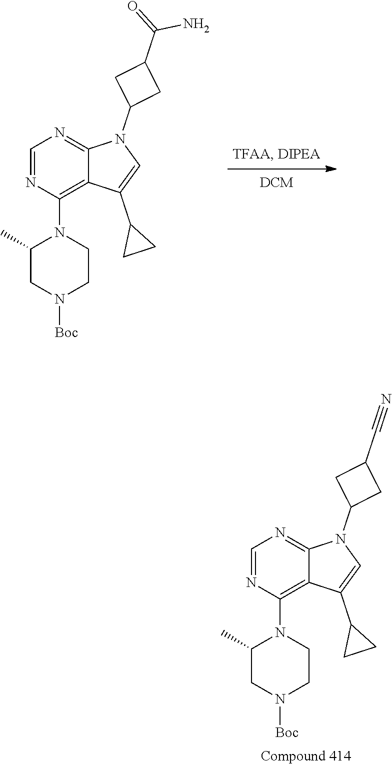

### Step 1. tert-Butyl (S)-4-(5-cyclopropyl-7-(3-(methoxycarbonyl)cyclobutyl)-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (360 mg, 1.0 mmol, prepared following the procedure of compound 260, step 4) and methyl 3-hydroxycyclobutane-1-carboxylate (260 mg, 2.0 mmol) in dry toluene (5 mL) were added CMBP (480 mg, 2.0 mmol) under N₂ atmosphere. The resulting mixture was stirred at 100° C. under N₂ atmosphere overnight. After being cooled down to room temperature, the reaction was partitioned between EtOAc and water, organic layer separated, aqueous layer extracted with EtOAc twice, the combined organic layers washed with brine, dried over Na₂SO₄, filtered and concentrated. The residue was purified by flash column chromatography (silica gel, 0-40%, ethyl acetate in petroleum ether) to afford tert-butyl (S)-4-(5-cyclopropyl-7-(3-(methoxycarbonyl)cyclobutyl)-7H-pyrrolo[2,3-d]pyrimidin-4-yl)-3-methylpiperazine-1-carboxylate (330 mg, 71%) as a white solid. LC/MS ESI (m/z): 470 (M+H)⁺.

### Step 2. (S)-3-(4-(4-(tert-Butoxycarbonyl)-2-methylpiperazin-1-yl)-5-cyclopropyl-7H-pyrrolo[2,3-d]pyrimidin-7-yl)cyclobutane-1-carboxylic acid

To a solution of tert-butyl (S)-4-(5-cyclopropyl-7-(3-(methoxycarbonyl)cyclobutyl)-7H-pyrrolo[2,3-d]pyrimidin-4-yl)-3-methylpiperazine-1-carboxylate (300 mg, 0.64 mmol) in H₂O (5 mL) and THE (5 mL) was added LiOH (330 mg, 8.0 mmol) and the reaction was stirred for 3 h at rt. The reaction was partitioned between EtOAc and water, aqueous layer separated, organic layer was extracted with NaHCO₃ twice. The combined aqueous layers were neutralized with HCl (1.0 M) and stirred with EtOAc. The organic layers were separated, aqueous layer extracted with EtOAc twice, the combined organic layers washed with brine, dried over Na₂SO₄, filtered and concentrated to afford (S)-3-(4-(4-(tert-butoxycarbonyl)-2-methylpiperazin-1-yl)-5-cyclopropyl-7H-pyrrolo[2,3-d]pyrimidin-7-yl)cyclobutane-1-carboxylic acid as a white solid (140 mg, 47% yield). LC/MS ESI (m/z): 456 (M+H)⁺.

### Step 3. tert-Butyl (S)-4-(7-(3-carbamoylcyclobutyl)-5-cyclopropyl-7H-pyrrolo[2,3-d]pyrimidin-4-yl)-3-methylpiperazine-1-carboxylate

To a solution of (S)-3-(4-(4-(tert-butoxycarbonyl)-2-methylpiperazin-1-yl)-5-cyclopropyl-7H-pyrrolo[2,3-d]pyrimidin-7-yl)cyclobutane-1-carboxylic acid (130 mg, 0.30 mmol) in DMF (2 mL) and DIPEA (0.40 mL) was added NH₄Cl (60 mg, 1.1 mmol), HOBT (110 mg, 0.84 mmol) and EDCI (160 mg, 0.84 mol) and stirred at room temperature overnight. The reaction was quenched with NaHCO₃ aqueous, diluted with brine and layers were separated. The organic layers were washed with brine, dried over Na₂SO₄, filtered and concentrated to afford crude tert-butyl (S)-4-(7-(3-carbamoylcyclobutyl)-5-cyclopropyl-7H-pyrrolo[2,3-d]pyrimidin-4-yl)-3-methylpiperazine-1-carboxylate as an oil (150 mg, contained trapped DMF). LC/MS ESI (m/z): 455 (M+H)⁺.

### Step 4. tert-Butyl (S)-4-(7-(3-cyanocyclobutyl)-5-cyclopropyl-7H-pyrrolo[2,3-d]pyrimidin-4-yl)-3-methylpiperazine-1-carboxylate

To a solution of tert-butyl (S)-4-(7-(3-carbamoylcyclobutyl)-5-cyclopropyl-7H-pyrrolo[2,3-d]pyrimidin-4-yl)-3-methylpiperazine-1-carboxylate (90 mg, contained trapped DMF) in DCM (4 mL) maintained in an ice bath and DIPEA (520 mg, 0.66 mL, 4.0 mmol) was added TFAA (420 mg, 0.27 mL, 2.0 mmol) and stirred at 0° C. for 2 h. The reaction was quenched with NaHCO₃ aqueous, diluted with brine and layers were separated. The organic layers were washed with brine, dried over Na₂SO₄, filtered and concentrated. The residue was purified by flash column chromatography (silica gel, 0˜30%, ethyl acetate in petroleum ether) to afford a crude product which was further purified by prep-HPLC to afford tert-butyl (S)-4-(7-(3-cyanocyclobutyl)-5-cyclopropyl-7H-pyrrolo[2,3-d]pyrimidin-4-yl)-3-methylpiperazine-1-carboxylate as a white solid (19 mg, 21%). LC/MS ESI (m/z): 437 (M+H)⁺. ¹H NMR (400 MHz, CDCl₃) δ 8.34 (s, 1H), 6.65 (s, 1H), 5.51-5.33 (m, 1H), 4.81-4.61 (m, 1H), 4.20-3.71 (m, 3H), 3.49 (t, J=11.1 Hz, 1H), 3.40-3.20 (m, 2H), 3.18-2.99 (m, 3H), 2.94-2.82 (m, 2H), 2.02-1.87 (m, 1H), 1.48 (s, 9H), 1.19 (d, J=6.3 Hz, 3H), 1.02-0.90 (m, 2H), 0.78-0.67 (m, 1H), 0.66-0.55 (m, 1H).

### Example 115. Synthesis of tert-butyl (S)-4-(7-(4-cyanopyridin-2-yl)-5-((R)-2-methylmorpholino)-7H-pyrrolo[2,3-d]pyrimidin-4-yl)-3-methylpiperazine-1-carboxylate (Compound 417)

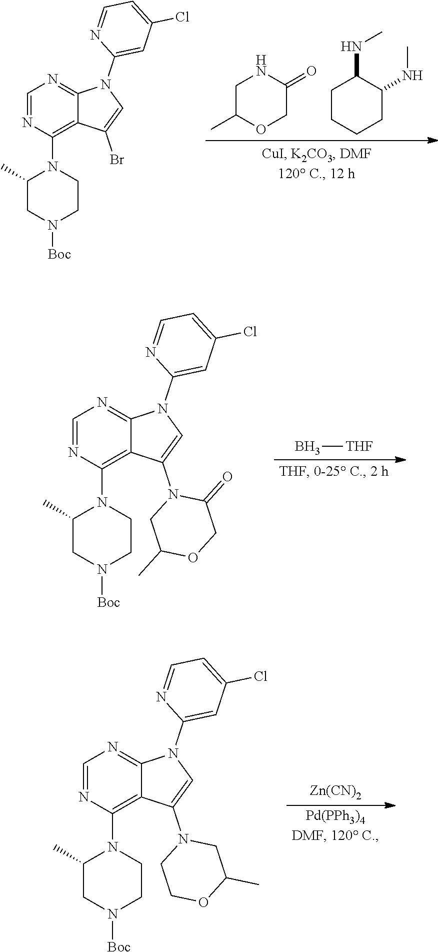

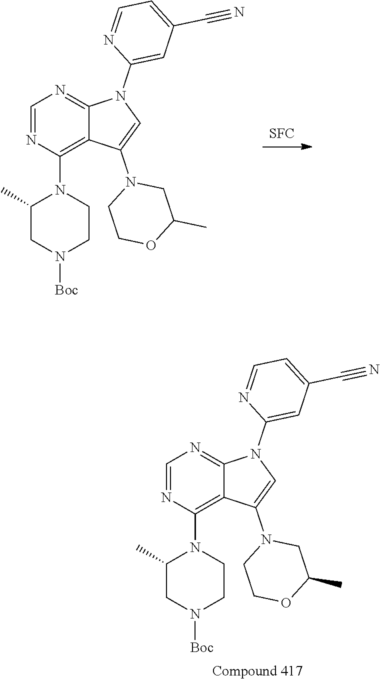 [SMILES: CC1CN(c2cn(-c3cc(C#N)ccn3)c3ncnc(N4CCN(C(=O)OC(C)(C)C)C[C@@H]4C)c23)CCO1 ; "SFC" ; C[C@@H]1CN(c2cn(-c3cc(C#N)ccn3)c3ncnc(N4CCN(C(=O)OC(C)(C)C)C[C@@H]4C)c23)CCO1]

### Step 1. tert-Butyl (S)-4-(5-bromo-7-(4-chloropyridin-2-yl)-7H-pyrrolo[2,3-d]pyrimidin-4-yl)-3-methylpiperazine-1-carboxylate

To a solution of tert-butyl (S)-4-(5-bromo-7-(4-chloropyridin-2-yl)-7H-pyrrolo[2,3-d]pyrimidin-4-yl)-3-methylpiperazine-1-carboxylate (100 mg, 0.20 mmol, prepared following the procedure outlined in compound 388) in DMF (5.0 mL) was added CuI (19 mg, 0.10 mmol), 6-methylmorpholin-3-one (45 mg, 0.40 mmol) and K₂CO₃ (54 mg, 0.40 mmol) at 25° C. The mixture was degassed with N₂ three times. The mixture was then heated to 120° C. and stirred at 120° C. for 12 hrs. The mixture was cooled to 25° C. and poured into H₂O (20 mL). The mixture was extracted with EtOAc twice, organic layers washed with brine, dried over Na₂SO₄, filtered and concentrated in vacuo. The crude product was purified by column chromatography (silica gel, 20-25% petroleum ether/ethyl acetate) to afford tert-butyl (3S)-4-(7-(4-chloropyridin-2-yl)-5-(2-methyl-5-oxomorpholino)-7H-pyrrolo[2,3-d]pyrimidin-4-yl)-3-methylpiperazine-1-carboxylate (60 mg, 55%) as a white solid. LC/MS ESI (m/z): 542 (M+H)⁺.

### Step 2. tert-Butyl (3S)-4-(7-(4-chloropyridin-2-yl)-5-(2-methylmorpholino)-7H-pyrrolo[2,3-d]pyrimidin-4-yl)-3-methylpiperazine-1-carboxylate

To a solution of tert-butyl (3S)-4-(7-(4-chloropyridin-2-yl)-5-(2-methyl-5-oxomorpholino)-7H-pyrrolo[2,3-d]pyrimidin-4-yl)-3-methylpiperazine-1-carboxylate (60 mg, 0.11 mmol) in THE (5 mL) was added BH₃-THF (1.0 mL, 1.0M in THF) at 0° C. under N₂. The mixture was warmed to 25° C. and then stirred for 2 hrs. The reaction was quenched by addition of MeOH (10.0 mL) at 0° C. and concentrated in vacuo. The crude product was purified by column chromatography (silica gel, 50:1-40:1 petroleum ether/ethyl acetate) to afford tert-butyl (3S)-4-(7-(4-chloropyridin-2-yl)-5-(2-methylmorpholino)-7H-pyrrolo[2,3-d]pyrimidin-4-yl)-3-methylpiperazine-1-carboxylate (40 mg, 68%) as a white solid. LC/MS ESI (m/z): 528 (M+H)⁺.

### Step 3. tert-Butyl (3S)-4-(7-(4-cyanopyridin-2-yl)-5-(2-methylmorpholino)-7H-pyrrolo[2,3-d]pyrimidin-4-yl)-3-methylpiperazine-1-carboxylate

To a solution of tert-butyl (3S)-4-(7-(4-chloropyridin-2-yl)-5-(2-methylmorpholino)-7H-pyrrolo[2,3-d]pyrimidin-4-yl)-3-methylpiperazine-1-carboxylate (40 mg, 0.076 mmol) in DMF (3 mL) was added Zn(CN)₂ (53 mg, 0.45 mmol) and Pd(PPh₃)₄ (44 mg, 0.040 mmol) at 25° C. The mixture was degassed with N₂ three times and then heated to 120° C. for 3 hrs. The mixture was poured into H₂O (10 mL) and extracted with EtOAc twice. The combined organic layers were washed with brine, dried over Na₂SO₄, filtered and concentrated in vacuo. The crude product was purified by column chromatography (silica gel, 50:1-30:1 petroleum ether/ethyl acetate) and Prep-HPLC to afford tert-butyl (3S)-4-(7-(4-cyanopyridin-2-yl)-5-(2-methylmorpholino)-7H-pyrrolo[2,3-d]pyrimidin-4-yl)-3-methylpiperazine-1-carboxylate (10 mg, 26%) as a yellow solid. LC/MS ESI (m/z): 519 (M+H)⁺.

### Step 4. tert-Butyl (S)-4-(7-(4-cyanopyridin-2-yl)-5-((R)-2-methylmorpholino)-7H-pyrrolo[2,3-d]pyrimidin-4-yl)-3-methylpiperazine-1-carboxylate

Racemic tert-butyl (3S)-4-(7-(4-cyanopyridin-2-yl)-5-(2-methylmorpholino)-7H-pyrrolo[2,3-d]pyrimidin-4-yl)-3-methylpiperazine-1-carboxylate (10 mg, 0.019 mmol) was separated by chiral Prep-HPLC (IA-H 4.6*250 mm IPA+0.05% DEA 40% 8 min). The longer retention time peak was labeled tert-butyl (S)-4-(7-(4-cyanopyridin-2-yl)-5-((R)-2-methylmorpholino)-7H-pyrrolo[2,3-d]pyrimidin-4-yl)-3-methylpiperazine-1-carboxylate (2.0 mg, 0.004 mmol) and was obtained as a white solid. LC/MS ESI (m/z): 519 (M+H)⁺. ¹H NMR (400 MHz, CDCl₃) δ 9.30 (s, 1H), 8.52 (d, J=4.8 Hz, 1H), 8.39 (s, 1H), 7.57 (s, 1H), 7.25 (d, J=5.0 Hz, 1H), 4.98 (s, 1H), 4.29-4.04 (m, 2H), 3.93-3.69 (m, 4H), 3.43-3.30 (m, 2H), 3.28-3.11 (m, 2H), 2.80 (m, 1H), 2.55-2.33 (m, 2H), 1.43 (s, 9H), 1.10-1.05 (m, 3H), 0.83-0.76 (m, 3H)

### Example 116. Synthesis of tert-butyl (R)-4-(3-cyclopropyl-1-(3,5-difluorophenyl)-1H-pyrrolo[3,2-c]pyridin-4-yl)-2-methylpiperazine-1-carboxylate (Compound 418)

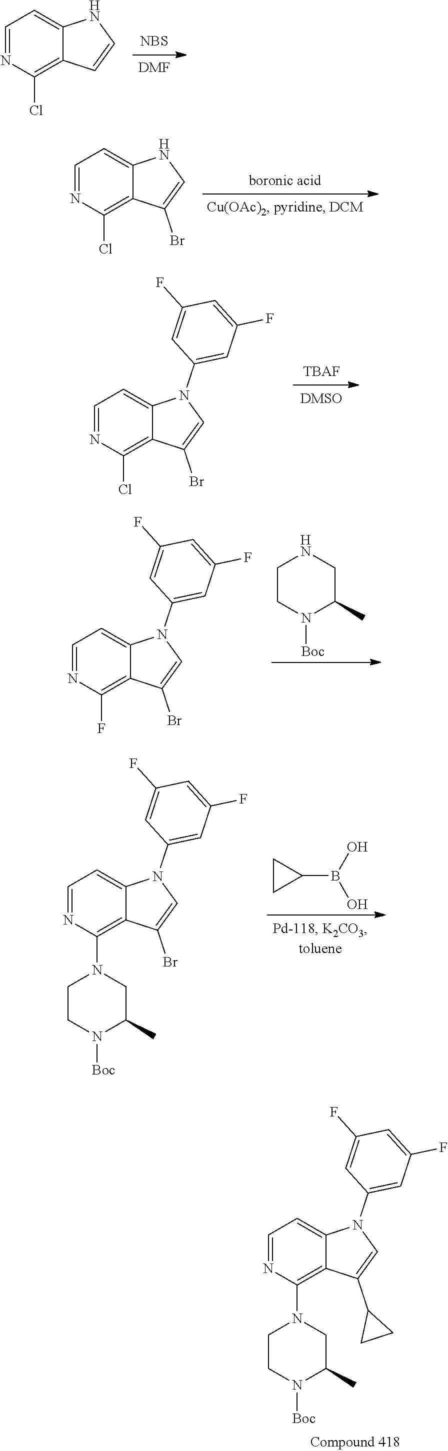

### Step 1. 3-Bromo-4-chloro-1H-pyrrolo[3,2-c]pyridine

To a solution of 4-chloro-1H-pyrrolo[3,2-c]pyridine (1.0 g, 6.6 mmol) in DMF (15 mL) at 0° C. was added NBS (1.7 g, 9.8 mmol) in portions. After the addition, the mixture was stirred at room temperature for 12 hrs. The reaction was quenched with saturated Na₂SO₃ solution at 0° C., and then was extracted with EtOAc twice. The combined organic layers were washed with brine, dried over Na₂SO₄ and concentrated to give the 3-bromo-4-chloro-1H-pyrrolo[3,2-c]pyridine (1.4 g, 94%) as a light yellow solid which was used directly in next step without further purification. LC/MS ESI (m/z): 231, 233 (M+H)⁺.

### Step 2. 3-Bromo-4-chloro-1-(3,5-difluorophenyl)-1H-pyrrolo[3,2-c]pyridine

A mixture of 3-bromo-4-chloro-1H-pyrrolo[3,2-c]pyridine (1.4 g, 6.0 mmol), (3,5-difluorophenyl)boronic acid (2.9 g, 18 mmol), Cu(OAc)₂ (2.8 g, 15 mmol) and pyridine (3.0 mL, 37 mmol) in DCM (50 mL) was stirred at room temperature for 48 hrs under an oxygen atmosphere. The reaction mixture was treated with ammonia, filtered, the filtrate extracted with DCM twice. The combined organic layers were washed with brine, dried over Na₂SO₄ and concentrated. The residue was purified by column chromatography (silica gel, 0˜30% ethyl acetate in petroleum ether, v/v) to give 3-bromo-4-chloro-1-(3,5-difluorophenyl)-1H-pyrrolo[3,2-c]pyridine (560 mg, 27%) as a solid. LC/MS ESI (m/z): 343, 345 (M+H)⁺.

### Step 3. 3-Bromo-1-(3,5-difluorophenyl)-4-fluoro-1H-pyrrolo[3,2-c]pyridine

To a solution of 3-bromo-4-chloro-1-(3,5-difluorophenyl)-1H-pyrrolo[3,2-c]pyridine (100 mg, 0.30 mmol) in DMSO (2 mL) was added TBAF (4.0 mL, 1.0 M in THF). The resulting mixture was heated at 150° C. for 2 days. After being cooled down to room temperature, the reaction was quenched with ice water, extracted with EtOAc twice, the combined organic layers washed with brine, dried over Na₂SO₄, filtered and concentrated. The residue was purified by flash column chromatography (silica gel, 0˜30%, ethyl acetate in petroleum ether) to afford 3-bromo-1-(3,5-difluorophenyl)-4-fluoro-1H-pyrrolo[3,2-c]pyridine (50 mg, 53%) as a yellow solid. LC/MS ESI (m/z): 327, 329 (M+H)⁺.

### Step 4. tert-Butyl (R)-4-(3-bromo-1-(3,5-difluorophenyl)-1H-pyrrolo[3,2-c]pyridin-4-yl)-2-methylpiperazine-1-carboxylate

A mixture of 3-bromo-1-(3,5-difluorophenyl)-4-fluoro-1H-pyrrolo[3,2-c]pyridine (50 mg, 0.15 mmol) and tert-butyl (R)-2-methylpiperazine-1-carboxylate (310 mg, 1.5 mmol). was heated at 150° C. for 3 h. After cooling to room temperature, the reaction mixture was purified by flash column chromatography (silica gel, 0˜35%, ethyl acetate in petroleum ether) to afford tert-butyl (R)-4-(3-bromo-1-(3,5-difluorophenyl)-1H-pyrrolo[3,2-c]pyridin-4-yl)-2-methylpiperazine-1-carboxylate (30 mg, 38%) as a solid. LC/MS ESI (m/z): 507, 509 (M+H)⁺.

### Step 5. tert-Butyl (R)-4-(3-cyclopropyl-1-(3,5-difluorophenyl)-1H-pyrrolo[3,2-c]pyridin-4-yl)-2-methylpiperazine-1-carboxylate

To a solution of tert-butyl (R)-4-(3-bromo-1-(3,5-difluorophenyl)-1H-pyrrolo[3,2-c]pyridin-4-yl)-2-methylpiperazine-1-carboxylate (30 mg, 0.060 mmol) in toluene (15 mL) were added cyclopropylboronic acid (51 mg, 0.60 mmol), K₂CO₃ (160 mg, 1.2 mmol) and Pd-118 (19 mg, 0.030 mmol). The resulting mixture was heated to 80° C. overnight. After cooling to room temperature, the reaction mixture was filtered. The filtrate was concentrated and purified by flash column chromatography (silica gel, 0˜20%, ethyl acetate in petroleum ether) to give crude product which was further purified by prep-HPLC to afford tert-butyl (R)-4-(3-cyclopropyl-1-(3,5-difluorophenyl)-1H-pyrrolo[3,2-c]pyridin-4-yl)-2-methylpiperazine-1-carboxylate (6.8 mg, 24%) as a white solid. LC/MS ESI (m/z): 469 (M+H)⁺. ¹H NMR (400 MHz, CDCl₃) δ 8.02 (d, J=6.0 Hz, 1H), 7.11 (d, J=6.0 Hz, 1H), 6.98 (dd, J=9.9, 4.4 Hz, 2H), 6.87-6.80 (m, 2H), 4.49-4.42 (m, 1H), 4.10-4.03 (m, 1H), 4.00-3.94 (m, 1H), 3.61-3.54 (m, 1H), 3.44-3.30 (m, 2H), 2.91-2.79 (m, 1H), 2.40-2.33 (m, 1H), 1.50 (s, 9H), 1.31 (d, J=6.7 Hz, 3H), 1.09-1.03 (m, 2H), 0.91-0.86 (m, 1H), 0.65-0.58 (m, 1H).

The following compound was prepared analogous to the synthesis of compound 418 from the corresponding amine and aryl halide.

| Cmpd No. | Chemical Name | LCMS and ¹H NMR |
|---|---|---|
| 156 | tert-butyl 4-(1-(4-chlorophenyl)- | LC/MS ESI (m/z): 453 (M + H)⁺. ¹H NMR (400 MHz, |
| | 3-cyclopropyl-1H-pyrrolo[3,2- | CDCl3) δ 7.96 (d, J = 5.9 Hz, 1H), 7.50-7.45 (m, 2H), |
| | c]pyridin-4-yl)piperazine-1- | 7.37-7.32 (m, 2H), 7.00 (d, J = 5.9 Hz, 1H), 6.79 (s, 1H), |
| | carboxylate | 3.68-3.60 (m, 4H), 3.45-3.37 (m, 4H), 2.43-2.35 (m, |
| | | 1H), 1.50 (s, 9H), 1.05-0.99 (m, 2H), 0.74-0.69 (m, 2H). |

### Example 117. Synthesis of tert-butyl (S)-4-(7-(4-cyanopyridin-2-yl)-5-((R)-3-methylmorpholino)-7H-pyrrolo[2,3-d]pyrimidin-4-yl)-3-methylpiperazine-1-carboxylate (Compound 419)

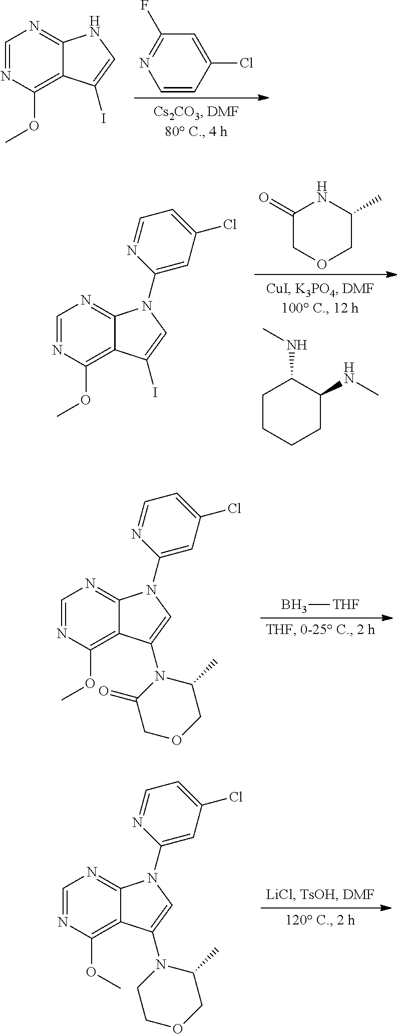

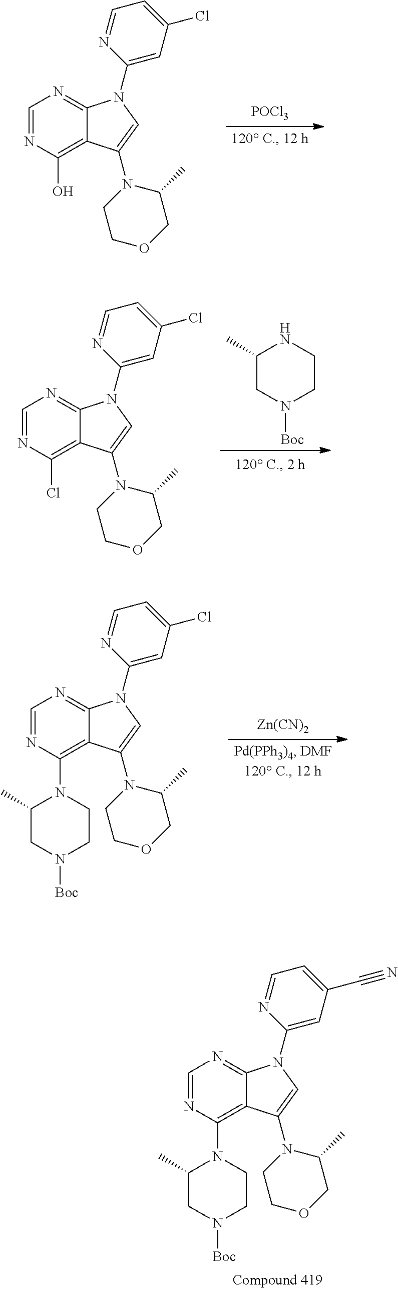

### Step 1. 7-(4-Chloropyridin-2-yl)-5-iodo-4-methoxy-7H-pyrrolo[2,3-d]pyrimidine

To a solution of 5-iodo-4-methoxy-7H-pyrrolo[2,3-d]pyrimidine (1.0 g, 3.6 mmol) in DMF (10 mL) was added CS₂CO₃ (2.4 g, 7.3 mmol) and 4-chloro-2-fluoropyridine (0.96 g, 7.3 mmol) at 25° C. The mixture was heated to 80° C. and stirred for 4 hrs. The mixture was cooled to 25° C. and poured into H₂O (50 mL) extracted with EtOAc (50 mL×4). The combined organic phase was washed with brine, dried over Na₂SO₄, filtered, and concentrated. The crude product was purified by silica gel column chromatography (SiO₂, petroleum ether:EtOAc 30:1) to give 7-(4-chloropyridin-2-yl)-5-iodo-4-methoxy-7H-pyrrolo[2,3-d]pyrimidine (1.2 g, 3.1 mmol) as a white solid. LC/MS ESI (m/z): 387 (M+H)⁺.

### Step 2. (R)-4-(7-(4-Chloropyridin-2-yl)-4-methoxy-7H-pyrrolo[2,3-d]pyrimidin-5-yl)-5-methylmorpholin-3-one

To a solution of 7-(4-chloropyridin-2-yl)-5-iodo-4-methoxy-7H-pyrrolo[2,3-d]pyrimidine (1.0 g, 2.6 mmol) in DMF (10 mL) was added (6R)-6-methylpiperidin-2-one (0.59 g, 5.2 mmol), K₃PO₄ (1.1 g, 5.2 mmol), CuI (0.088 mL, 2.6 mmol) and trans-N,N-dimethyl-1,2-cyclohexanediamine (0.74 g, 5.2 mmol) at 25° C. The mixture was degassed with N₂ 3 times. The mixture was heated to 120° C. and stirred for 12 hrs. The mixture was cooled to 25° C. and poured into H₂O (50 mL). The mixture was extracted with EtOAc (50 mL×4). The combined organic phase was washed with brine, dried over Na₂SO₄, filtered, and concentrated. The crude product was purified by silica gel column (SiO₂, petroleum ether:EtOAc=10:1) to provide (R)-4-(7-(4-chloropyridin-2-yl)-4-methoxy-7H-pyrrolo[2,3-d]pyrimidin-5-yl)-5-methylmorpholin-3-one (50 mg, 5.0%) as a white solid. LC/MS ESI (m/z): 374 (M+H)⁺.

### Step 3. (R)-4-(7-(4-Chloropyridin-2-yl)-4-methoxy-7H-pyrrolo[2,3-d]pyrimidin-5-yl)-3-methylmorpholine

To a solution of (R)-4-(7-(4-chloropyridin-2-yl)-4-methoxy-7H-pyrrolo[2,3-d]pyrimidin-5-yl)-5-methylmorpholin-3-one (50 mg, 0.13 mmol) in THE (2 mL) was added BH₃-THF (1.0 mL, 1.0M in THF) at 0° C. The mixture was warmed to 25° C. and stirred for 2 hrs. The reaction was quenched by addition of MeOH (5 mL) at 0° C. The mixture was concentrated, and the product was purified by silica gel column chromatography (SiO₂, petroleum ether:EtOAc=50:1 to 30:1) to provide (R)-4-(7-(4-chloropyridin-2-yl)-4-methoxy-7H-pyrrolo[2,3-d]pyrimidin-5-yl)-3-methylmorpholine (40 mg, 85%) as a white solid. LC/MS ESI (m/z): 360 (M+H)⁺.

### Step 4. (R)-7-(4-Chloropyridin-2-yl)-5-(3-methylmorpholino)-7H-pyrrolo[2,3-d]pyrimidin-4-ol

To a solution of (R)-4-(7-(4-chloropyridin-2-yl)-4-methoxy-7H-pyrrolo[2,3-d]pyrimidin-5-yl)-3-methylmorpholine (40 mg, 0.11 mmol) in DMF (5 mL) was added LiCl (4.7 mg, 0.11 mmol) and p-toluenesulfonic acid (0.18 mg, 1.1 mmol) at 25° C. The mixture was degassed with N₂ 3 times. The mixture was heated to 120° C. then cooled to 25° C. and poured into H₂O (30 mL). The mixture was extracted with EtOAc (50 mL×4). The combined organic phase was washed with brine, dried over Na₂SO₄, filtered, and concentrated. The product (R)-7-(4-chloropyridin-2-yl)-5-(3-methylmorpholino)-7H-pyrrolo[2,3-d]pyrimidin-4-ol (60 mg) was carried directly to the next step without further purification. LC/MS ESI (m/z): 346 (M+H)⁺.

### Step 5. (R)-4-(4-Chloro-7-(4-chloropyridin-2-yl)-7H-pyrrolo[2,3-d]pyrimidin-5-yl)-3-methylmorpholine

To a solution of (R)-7-(4-chloropyridin-2-yl)-5-(3-methylmorpholino)-7H-pyrrolo[2,3-d]pyrimidin-4-ol (0.11 mmol, theoretical) in POCl₃ (2.0 mL, 22 mmol) was heated to 120° C. and stirred for 12 hrs. The mixture was concentrated under vacuo to remove POCl₃. The residue was diluted with EtOAc (10 mL) and washed with H₂O (10 m1×3). The combined organic phase was washed with brine, dried over Na₂SO₄, filtered, and concentrated. Purification by silica gel column chromatography (SiO₂, petroleum ether:EtOAc=50:1 to 30:1) provided (R)-4-(4-chloro-7-(4-chloropyridin-2-yl)-7H-pyrrolo[2,3-d]pyrimidin-5-yl)-3-methyl morpholine (30 mg, 75%, over 2 steps) as a white solid. LC/MS ESI (m/z): 364 (M+H)⁺.

### Step 6. tert-Butyl (S)-4-(7-(4-chloropyridin-2-yl)-5-((R)-3-methylmorpholino)-7H-pyrrolo[2,3-d]pyrimidin-4-yl)-3-methylpiperazine-1-carboxylate

To a solution of (R)-4-(4-chloro-7-(4-chloropyridin-2-yl)-7H-pyrrolo[2,3-d]pyrimidin-5-yl)-3-methylmorpholine (30 mg, 0.082 mmol) in DIEA (1.0 mL) was added tert-butyl (3S)-3-methylpiperazine-1-carboxylate (36 mg, 0.33 mmol) at 25° C. The mixture was heated 120° C. and stirred for 2 hrs. The mixture was concentrated under vacuo. The crude product was purified by silica gel column chromatography (SiO₂, petroleum ether:EtOAc=50:1 to 30:1) to provide tert-butyl (S)-4-(7-(4-chloropyridin-2-yl)-5-((R)-3-methylmorpholino)-7H-pyrrolo[2,3-d]pyrimidin-4-yl)-3-methylpiperazine-1-carboxylate (15 mg, 34%) as a white solid. LC/MS ESI (m/z): 528 (M+H)⁺.

### Step 7. tert-Butyl (S)-4-(7-(4-cyanopyridin-2-yl)-5-((R)-3-methylmorpholino)-7H-pyrrolo[2,3-d]pyrimidin-4-yl)-3-methylpiperazine-1-carboxylate

To a solution of tert-butyl (S)-4-(7-(4-chloropyridin-2-yl)-5-((R)-3-methylmorpholino)-7H-pyrrolo[2,3-d]pyrimidin-4-yl)-3-methylpiperazine-1-carboxylate (15 mg, 0.028 mmol) in DMF (5 mL) was added Zn(CN)₂ (20 mg, 0.17 mmol) and Pd(PPh₃)₄ (33 mg, 0.028 mmol) at 25° C. The mixture was degassed with N₂ 3 times. The mixture was heated to 120° C. and stirred for 3 hrs. The mixture was cooled to 25° C. and poured into H₂O (10 mL). The mixture was extracted with EtOAc (50 mL×4). The combined organic phase was washed with brine, dried over Na₂SO₄, filtered, and concentrated. The product was purified by silica gel column (SiO₂, petroleum ether:EtOAc=50:1 to 30:1) followed by Prep-HPLC (0.1% formic acid) to provide tert-butyl (S)-4-(7-(4-cyanopyridin-2-yl)-5-((R)-3-methylmorpholino)-7H-pyrrolo[2,3-d]pyrimidin-4-yl)-3-methylpiperazine-1-carboxylate (1.0 mg, 6.9%) as a white solid. LC/MS ESI (m/z): 519 (M+H)⁺. ¹H NMR (400 MHz, CDCl₃) δ 9.38 (s, 1H), 8.59 (d, J=4.9 Hz, 1H), 8.43 (s, 1H), 7.88 (s, 1H), 7.34 (d, J=5.0 Hz, 1H), 5.29-4.54 (m, 2H), 4.35-4.04 (m, 1H), 3.99-3.85 (m, 3H), 3.84-3.74 (m, 1H), 3.50-3.39 (m, 1H), 3.36-3.27 (m, 1H), 3.23-3.03 (m, 4H), 2.93 (s, 1H), 1.50 (s, 9H), 1.26 (d, J=6.3 Hz, 3H), 0.98 (d, J=6.2 Hz, 3H).

The following compound was prepared an analogous procedure to the synthesis of compound 419 from the corresponding acid anhydride.

| Cmpd No. | Chemical Name | LCMS and ¹H NMR |
|---|---|---|
| 416 | tert-butyl (R)-4-(7-(4- | LC/MS ESI (m/z): 489.1 (M + H)⁺. ¹H NMR (400 MHz, |
| | cyanopyridin-2-yl)-5- | CDCl₃) δ 9.36 (s, 1H), 8.57 (d, J = 5.0 Hz, 1H), 8.43 (s, |
| | (pyrrolidin-1-yl)-7H- | 1H), 7.56 (s, 1H), 7.30-7.28 (m, 1H), 4.80-4.72 (m, 1H), |
| | pyrrolo[2,3-d]pyrimidin-4-yl)-2- | 4.44-4.32 (m, 2H), 3.96-3.87 (m, 1H), 3.37-3.20 (m, |
| | methylpiperazine-1-carboxylate | 4H), 3.02-2.92 (m, 1H), 2.91-2.85 (m, 2H), 2.01-1.94 |
| | | (m, 4H), 1.49 (s, 9H), 1.07 (d, J = 6.7 Hz, 3H). |

### Example 118. Synthesis of tert-butyl (S)-4-(7-(4-cyanopyridin-2-yl)-5-(diethylamino)-7H-pyrrolo[2,3-d]pyrimidin-4-yl)-3-methylpiperazine-1-carboxylate (Compound 424) and 1,1,1-trifluoro-2-methylpropan-2-yl (S)-4-(7-(4-cyanopyridin-2-yl)-5-(diethylamino)-7H-pyrrolo[2,3-d]pyrimidin-4-yl)-3-methylpiperazine-1-carboxylate (Compound 422)

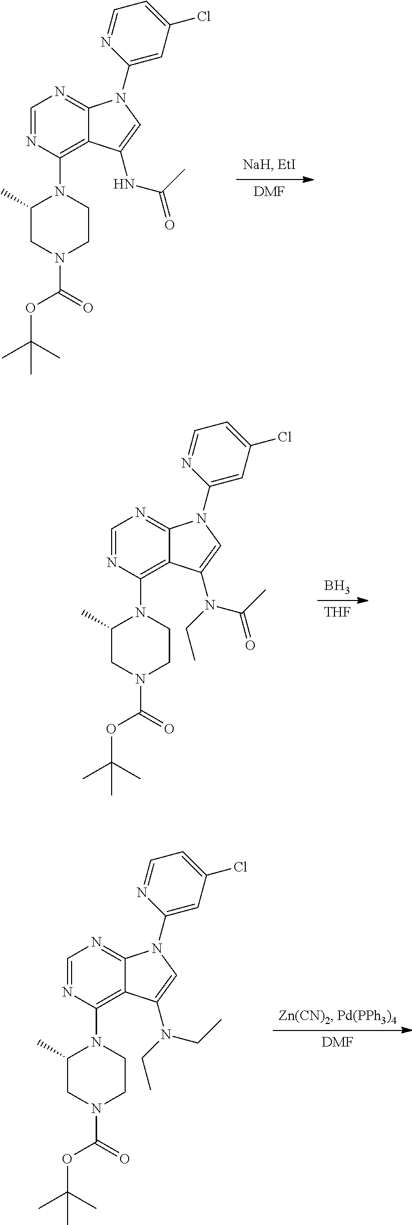

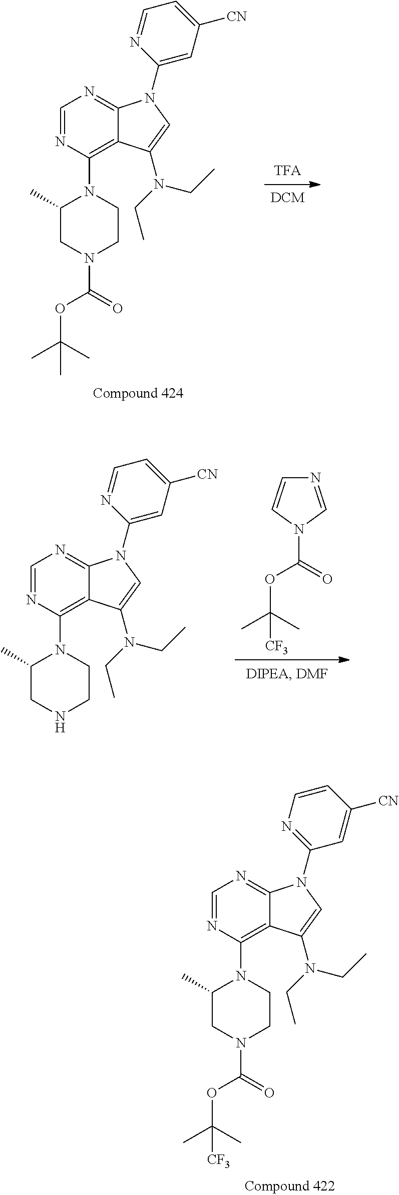

### Step 1. tert-Butyl (S)-4-(7-(4-chloropyridin-2-yl)-5-(N-ethylacetamido)-7H-pyrrolo[2,3-d]pyrimidin-4-yl)-3-methylpiperazine-1-carboxylate

At 0° C., to tert-butyl (S)-4-(5-acetamido-7-(4-chloropyridin-2-yl)-7H-pyrrolo[2,3-d]pyrimidin-4-yl)-3-methylpiperazine-1-carboxylate (400 mg, 0.82 mmol) in dry DMF (5 mL) under N₂ was added NaH (130 mg, 60% in mineral oil, 3.3 mmol). The mixture was stirred at rt for 15 min. Then CH₃CH₂I (260 mg, 1.7 mmol) was added. The mixture was stirred for an additional 20 min and was quenched with ice and extracted with EtOAc. The organic layer was washed with 5% LiCl (aq) and brine, dried over Na₂SO₄, and concentrated. The residue was purified by flash column chromatography (silica gel, 0˜33% EtOAc in petroleum ether) to provide tert-butyl (S)-4-(7-(4-chloropyridin-2-yl)-5-(N-ethylacetamido)-7H-pyrrolo[2,3-d]pyrimidin-4-yl)-3-methylpiperazine-1-carboxylate (280 mg, 66%) as a yellow foam. LC/MS ESI (m/z): 514 (M+H)⁺.

### Step 2. tert-Butyl (S)-4-(7-(4-chloropyridin-2-yl)-5-(diethylamino)-7H-pyrrolo[2,3-d]pyrimidin-4-yl)-3-methylpiperazine-1-carboxylate

At 0° C., to tert-butyl (S)-4-(7-(4-chloropyridin-2-yl)-5-(N-ethylacetamido)-7H-pyrrolo[2,3-d]pyrimidin-4-yl)-3-methylpiperazine-1-carboxylate (280 mg, 0.55 mmol) in dry THE (4 mL) was added BH₃-THF (8.0 mL, 1.0 M in THF) dropwise under N₂. The mixture was stirred at 0° C. for 2 h. Then it was carefully quenched with MeOH and concentrated. The residue was partitioned between EtOAc and brine. The organic layer was dried over Na₂SO₄ and purified by flash column chromatography (silica gel, 0˜13% EtOAc in petroleum ether) to provide tert-butyl (S)-4-(7-(4-chloropyridin-2-yl)-5-(diethylamino)-7H-pyrrolo[2,3-d]pyrimidin-4-yl)-3-methylpiperazine-1-carboxylate (160 mg, 56%) as a light yellow oil. LC/MS ESI (m/z): 500 (M+H)⁺.

### Step 3. tert-Butyl (S)-4-(7-(4-cyanopyridin-2-yl)-5-(diethylamino)-7H-pyrrolo[2,3-d]pyrimidin-4-yl)-3-methylpiperazine-1-carboxylate

In a sealed tube were added Zn(CN)₂ (90 mg, 0.77 mmol) and Pd(PPh₃)₄ (90 mg, 0.078 mmol). Then tert-butyl (S)-4-(7-(4-chloropyridin-2-yl)-5-(diethylamino)-7H-pyrrolo[2,3-d]pyrimidin-4-yl)-3-methylpiperazine-1-carboxylate (160 mg, 0.31 mmol) in dry DMF (3 mL) was added. The tube was purged with N₂ and sealed. The mixture was stirred at 120° C. for 2.5 h and was allowed to cool to rt. The mixture was partitioned between EtOAc and brine. The organic layer was washed with 5% LiCl (aq) and brine, dried over Na₂SO₄, and purified by flash column chromatography (silica gel, 0˜14% EtOAc in petroleum ether). Further purification by prep-HPLC provided tert-butyl (S)-4-(7-(4-cyanopyridin-2-yl)-5-(diethylamino)-7H-pyrrolo[2,3-d]pyrimidin-4-yl)-3-methylpiperazine-1-carboxylate (140 mg, 92%) as a yellow foam. 35 mg of this product was further purified by prep-HPLC and prep-TLC to provide 12 mg of the pure compound for submission. LC/MS ESI (m/z): 491 (M+H)⁺. ¹H NMR (400 MHz, CDCl₃) δ 9.38 (s, 1H), 8.57 (d, J=5.0 Hz, 1H), 8.43 (s, 1H), 7.62 (s, 1H), 7.29 (dd, J=5.0, 1.1 Hz, 1H), 5.13 (d, J=27.8 Hz, 1H), 4.45-3.80 (m, 3H), 3.38 (t, J=12.1 Hz, 1H), 3.30-3.10 (m, 5H), 3.01 (d, J=10.5 Hz, 1H), 1.50 (s, 9H), 1.16 (d, J=5.1 Hz, 3H), 1.02 (t, J=7.0 Hz, 6H).

### Step 4. (S)-2-(5-(Diethylamino)-4-(2-methylpiperazin-1-yl)-7H-pyrrolo[2,3-d]pyrimidin-7-yl)isonicotinonitrile

To tert-butyl (S)-4-(7-(4-cyanopyridin-2-yl)-5-(diethylamino)-7H-pyrrolo[2,3-d]pyrimidin-4-yl)-3-methylpiperazine-1-carboxylate (110 mg, 0.22 mmol) in DCM (8 mL) was added TFA (1.0 mL) dropwise at rt. The mixture was stirred at rt for 3 h and was diluted with DCM and quenched with NaHCO₃ (aq.). The organic layer was separated, washed with brine, dried over Na₂SO₄, filtered, and concentrated to give (S)-2-(5-(diethylamino)-4-(2-methylpiperazin-1-yl)-7H-pyrrolo[2,3-d]pyrimidin-7-yl)isonicotinonitrile which was used directly in the next step. LC/MS ESI (m/z): 391 (M+H)⁺.

### Step 5. 1,1,1-Trifluoro-2-methylpropan-2-yl (S)-4-(7-(4-cyanopyridin-2-yl)-5-(diethylamino)-7H-pyrrolo[2,3-d]pyrimidin-4-yl)-3-methylpiperazine-1-carboxylate

To (S)-2-(5-(diethylamino)-4-(2-methylpiperazin-1-yl)-7H-pyrrolo[2,3-d]pyrimidin-7-yl)isonicotinonitrile (50 mg, 0.13 mmol) and 1,1,1-trifluoro-2-methylpropan-2-yl 1H-imidazole-1-carboxylate (85 mg, 0.38 mmol) in DMF (2 mL) was added DIPEA (66 mg, 0.51 mmol). The mixture was stirred at 80° C. under N₂ for 24 h. The mixture was partitioned between EtOAc and brine. The organic layer was washed with 5% LiCl (aq) and brine, dried over Na₂SO₄, and concentrated. Purification by flash column chromatography (silica gel, 0-14% EtOAc in petroleum ether) followed by prep-HPLC provided 1,1,1-trifluoro-2-methylpropan-2-yl (S)-4-(7-(4-cyanopyridin-2-yl)-5-(diethylamino)-7H-pyrrolo[2,3-d]pyrimidin-4-yl)-3-methylpiperazine-1-carboxylate (34 mg, 48%) as a yellow solid. LC/MS ESI (m/z): 545 (M+H)⁺. ¹H NMR (400 MHz, CDCl₃) δ 9.38 (s, 1H), 8.58 (d, J=4.5 Hz, 1H), 8.44 (s, 1H), 7.63 (s, 1H), 7.30 (dd, J=5.0, 1.1 Hz, 1H), 5.15 (d, J=30.0 Hz, 1H), 4.51-4.23 (m, 1H), 4.09 (dd, J=67.8, 12.8 Hz, 1H), 3.88 (dd, J=40.2, 13.3 Hz, 1H), 3.45-3.26 (m, 2H), 3.24-2.99 (m, 5H), 1.72 (s, 6H), 1.16 (d, J=6.6 Hz, 3H), 1.02 (t, J=7.0 Hz, 6H).

### Example 119. Synthesis of tert-butyl (S)-4-(7-(4-cyanopyridin-2-yl)-5-(dimethylamino)-7H-pyrrolo[2,3-d]pyrimidin-4-yl)-3-methylpiperazine-1-carboxylate (Compound 426)

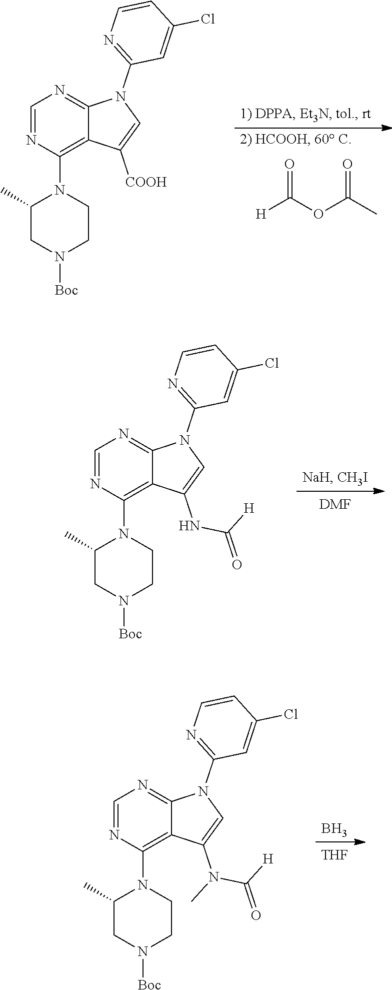

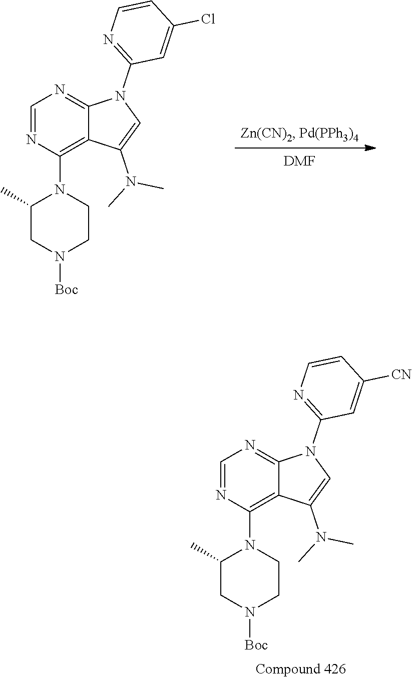

### Step 1. tert-Butyl (S)-4-(7-(4-chloropyridin-2-yl)-5-formamido-7H-pyrrolo[2,3-d]pyrimidin-4-yl)-3-methylpiperazine-1-carboxylate

The mixed anhydride was prepared by stirring a mixture of Ac₂O (2 mL) and HCOOH (0.9 mL) at 60° C. under N₂ for 2 h. To (S)-4-(4-(tert-butoxycarbonyl)-2-methylpiperazin-1-yl)-7-(4-chloropyridin-2-yl)-7H-pyrrolo[2,3-d]pyrimidine-5-carboxylic acid (400 mg, 0.85 mmol, prepared following the procedure outlined in compound 427 in toluene (9 mL) were added DPPA (300 mg, 1.1 mmol) and Et₃N (170 mg, 1.7 mmol). The mixture was stirred at rt under N₂ for 3 h. Then 1.5 mL of HCOOH and 1.6 mL of the mixed anhydride were added, and the mixture was heated to 60° C. and stirred for 1 h. NaHCO₃ (aq.) was added and the mixture was extracted with EtOAc, dried over Na₂SO₄ and concentrated. The residue was purified by flash column chromatography (silica gel, 0˜40% EtOAc in petroleum ether) to give tert-butyl (S)-4-(7-(4-chloropyridin-2-yl)-5-formamido-7H-pyrrolo[2,3-d]pyrimidin-4-yl)-3-methylpiperazine-1-carboxylate (280 mg, 70%) as a light yellow foam. LC/MS ESI (m/z): 472 (M+H)⁺.

### Step 2. tert-Butyl (S)-4-(7-(4-chloropyridin-2-yl)-5-(N-methylformamido)-7H-pyrrolo[2,3-d]pyrimidin-4-yl)-3-methylpiperazine-1-carboxylate

tert-Butyl (S)-4-(7-(4-chloropyridin-2-yl)-5-formamido-7H-pyrrolo[2,3-d]pyrimidin-4-yl)-3-methylpiperazine-1-carboxylate (280 mg, 0.59 mmol) in DMF (5 mL) was cooled in an ice bath under N₂ for 10 min. Then NaH (95 mg, 60% in mineral oil, 2.4 mmol) was added. The mixture was stirred at rt for 15 min. Then it was cooled in the ice bath and CH₃I (170 mg, 1.2 mmol) was added. After 30 min, the mixture was carefully quenched with ice. The mixture was extracted with EtOAc, washed with 5% LiCl (aq.) and brine, dried over Na₂SO₄, and concentrated to dryness to provide tert-butyl (S)-4-(7-(4-chloropyridin-2-yl)-5-(N-methylformamido)-7H-pyrrolo[2,3-d]pyrimidin-4-yl)-3-methylpiperazine-1-carboxylate (280 mg, 97%), which was used directly in the next step. LC/MS ESI (m/z): 486 (M+H)⁺.

### Step 3. tert-Butyl (S)-4-(7-(4-chloropyridin-2-yl)-5-(dimethylamino)-7H-pyrrolo[2,3-d]pyrimidin-4-yl)-3-methylpiperazine-1-carboxylate

Tert-butyl (S)-4-(7-(4-chloropyridin-2-yl)-5-(N-methylformamido)-7H-pyrrolo[2,3-d]pyrimidin-4-yl)-3-methylpiperazine-1-carboxylate (280 mg, 0.58 mmol) in dry THE (6 mL) was cooled in an ice-bath for 15 min. Then BH₃-Me₂S (3.0 mL, 2.0 M in THF) was added dropwise. The mixture was stirred at this temperature for 1.5 h and was carefully quenched with MeOH. The mixture was partitioned between EtOAc and H₂O. The organic layer was dried over Na₂SO₄ and purified by flash column chromatography (silica gel, 0˜14% EtOAc in petroleum ether) to provide tert-butyl (S)-4-(7-(4-chloropyridin-2-yl)-5-(dimethylamino)-7H-pyrrolo[2,3-d]pyrimidin-4-yl)-3-methylpiperazine-1-carboxylate (160 mg, 57%) as a light yellow foam. LC/MS ESI (m/z): 472 (M+H)⁺.

### Step 4. tert-Butyl (S)-4-(7-(4-cyanopyridin-2-yl)-5-(dimethylamino)-7H-pyrrolo[2,3-d]pyrimidin-4-yl)-3-methylpiperazine-1-carboxylate

In a sealed tube, to Zn(CN)₂ (95 mg, 0.81 mmol) and Pd(PPh₃)₄ (95 mg, 0.082 mmol) was added a solution of tert-butyl (S)-4-(7-(4-chloropyridin-2-yl)-5-(dimethylamino)-7H-pyrrolo[2,3-d]pyrimidin-4-yl)-3-methylpiperazine-1-carboxylate (160 mg, 0.33 mmol) in DMF (3 mL). The mixture was purged with N₂ and stirred at 120° C. for 3 h. The mixture was partitioned between EtOAc and brine. The organic layer was washed with LiCl (5% aq.) and brine and concentrated. Purification by flash column chromatography (silica gel, 0˜20% EtOAc in petroleum ether) provided tert-butyl (S)-4-(7-(4-cyanopyridin-2-yl)-5-(dimethylamino)-7H-pyrrolo[2,3-d]pyrimidin-4-yl)-3-methylpiperazine-1-carboxylate (150 mg, 95%) as a yellow foam. 60 mg of this product was further purified by prep-HPLC to give 40 mg of the pure product for submission. LC/MS ESI (m/z): 463 (M+H)⁺. ¹H NMR (400 MHz, CDCl₃) δ 9.36 (s, 1H), 8.58 (d, J=5.0 Hz, 1H), 8.44 (s, 1H), 7.57 (s, 1H), 7.30 (dd, J=5.0, 1.2 Hz, 1H), 5.04 (s, 1H), 4.35-3.84 (m, 3H), 3.42 (td, J=13.0, 3.2 Hz, 1H), 3.29 (s, 1H), 3.12-2.93 (m, 1H), 2.77 (s, 6H), 1.49 (s, 9H), 1.17 (d, J=6.6 Hz, 3H).

The following compound was prepared by an analogous synthetic procedure to that described for compound 426, except using (S)-4-(4-(tert-butoxycarbonyl)-2-methylpiperazin-1-yl)-7-(3-chlorophenyl)-7H-pyrrolo[2,3-d]pyrimidine-5-carboxylic acid as the starting material.

| Cmpd No. | Chemical Name | LCMS and ¹H NMR |
|---|---|---|
| 415 | tert-butyl (S)-4-(7-(3- | LC/MS ESI (m/z): 471 (M + H)⁺. ¹H NMR (400 MHz, |
| | chlorophenyl)-5- | CDCl₃) δ 8.38 (s, 1H), 7.72 (d, J = 1.7 Hz, 1H), 7.66 (dd, J |
| | (dimethylamino)-7H- | = 8.2, 0.8 Hz, 1H), 7.42 (t, J = 8.1 Hz, 1H), 7.29 (d, J = 0.8 |
| | pyrrolo[2,3-d]pyrimidin-4-yl)-3- | Hz, 1H), 6.71 (s, 1H), 5.05 (s, 1H), 4.36-3.84 (m, 3H), |
| | methylpiperazine-1-carboxylate | 3.42 (td, J = 13.2, 2.6 Hz, 1H), 3.17 (d, J = 112.4 Hz, 2H), |
| | | 2.76 (s, 6H), 1.50 (s, 9H), 1.17 (d, J = 6.6 Hz, 3H). |

### Example 120. tert-Butyl (R)-4-(7-(4-cyanopyridin-2-yl)-5-(dimethylamino)-7H-pyrrolo[2,3-d]pyrimidin-4-yl)-2-methylpiperazine-1-carboxylate (Compound 427)

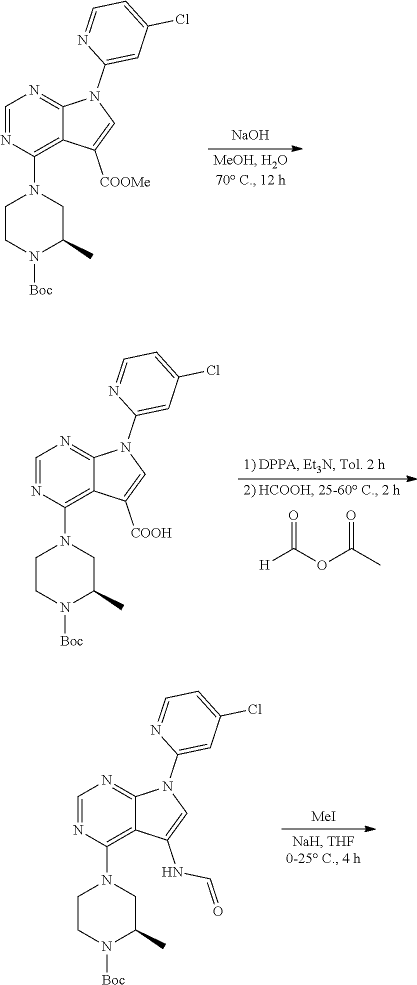

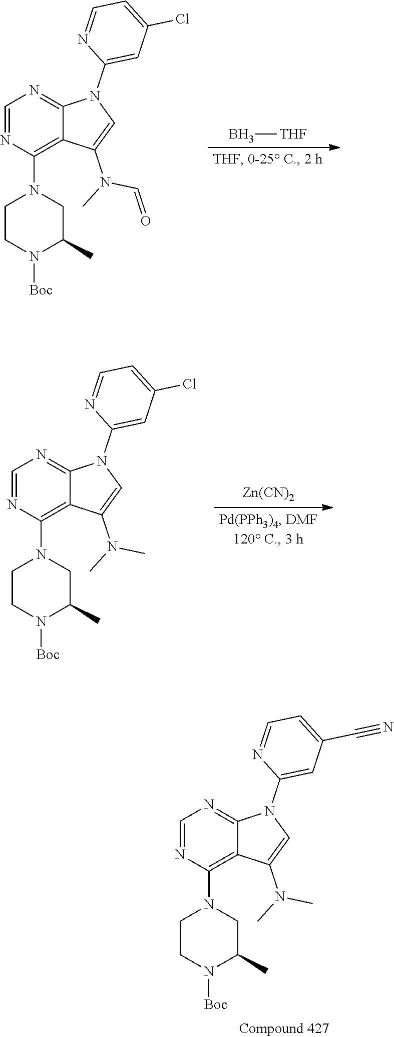

### Step 1. (R)-4-(4-(tert-Butoxycarbonyl)-3-methylpiperazin-1-yl)-7-(4-chloropyridin-2-yl)-7H-pyrrolo[2,3-d]pyrimidine-5-carboxylic acid

To a solution of methyl (R)-4-(4-(tert-butoxycarbonyl)-3-methylpiperazin-1-yl)-7-(4-chloropyridin-2-yl)-7H-pyrrolo[2,3-d]pyrimidine-5-carboxylate (2.0 g, 4.1 mmol, prepared following the procedure outlined in compound 443) in H₂O (4.0 mL) and MeOH (16 mL) was added NaOH (0.66 g, 16 mmol) at 25° C. The mixture was heated to 70° C. and stirred for 12 hrs. The mixture was cooled to 25° C. and poured into H₂O (40 mL) and adjusted to pH=7 with NaOH (2N). The mixture was extracted with EtOAc (50 mL×4). The combined organic phase was washed with brine, dried over Na₂SO₄, filtered, and concentrated. The crude product was purified by silica gel column chromatography (SiO₂, petroleum ether:EtOAc=50:1 to 30:1) to provide (R)-4-(4-(tert-butoxycarbonyl)-3-methylpiperazin-1-yl)-7-(4-chloropyridin-2-yl)-7H-pyrrolo[2,3-d]pyrimidine-5-carboxylic acid (800 mg, 41%). LC/MS ESI (m/z): 471 (M−H)⁻.

### Step 2. tert-Butyl(R)-4-(7-(4-cyanopyridin-2-yl)-5-(dimethylamino)-7H-pyrrolo[2,3-d]pyrimidin-4-yl)-2-methylpiperazine-1-carboxylate

To a solution of (R)-4-(4-(tert-butoxycarbonyl)-3-methylpiperazin-1-yl)-7-(4-chloropyridin-2-yl)-7H-pyrrolo[2,3-d]pyrimidine-5-carboxylic acid (200 mg, 0.42 mmol) in toluene (3 mL) was added Et₃N (86 mg, 0.85 mmol) and DPPA (0.18 mL, 0.85 mmol) at 25° C. The mixture was stirred at 25° C. for 2 hrs. Then HCOOH (39 mg, 0.85 mmol) was added to the mixture at 25° C. Formyl acetate (75 mg, 0.85 mmol) was added to the mixture at 25° C. The mixture was heated to 60° C. and stirred for 2 hrs then cooled to 25° C. The mixture was poured into H₂O (100 mL) and extracted with EtOAc (50 mL×4). The combined organic phase was washed with brine, dried over Na₂SO₄, filtered, and concentrated. The crude product was purified by silica gel column (SiO₂, petroleum ether:EtOAc=100:1 to 10:1) to give tert-butyl (R)-4-(7-(4-cyanopyridin-2-yl)-5-(dimethylamino)-7H-pyrrolo[2,3-d]pyrimidin-4-yl)-2-methylpiperazine-1-carboxylate (200 mg, 0.42 mmol). LC/MS ESI (m/z): 472 (M+H)⁺.

### Step 3. tert-Butyl (R)-4-(7-(4-chloropyridin-2-yl)-5-(N-methylformamido)-7H-pyrrolo[2,3-d]pyrimidin-4-yl)-2-methylpiperazine-1-carboxylate

To a solution of tert-butyl (R)-4-(7-(4-chloropyridin-2-yl)-5-formamido-7H-pyrrolo[2,3-d]pyrimidin-4-yl)-2-methylpiperazine-1-carboxylate (100 mg, 0.21 mmol) in THE (2 mL) was added NaH (17 mg, 0.42 mmol) at 0° C. The mixture was stirred at 0° C. for 1 hr and then MeI (60 mg, 0.42 mmol) was added to the mixture at 0° C. The mixture was warmed to 25° C. and stirred at 25° C. for 3 hrs. The mixture was quenched by addition sat. NH₄Cl (5.0 mL) and poured into H₂O (100 mL) and extracted with EtOAc (50.0 mL×4). The combined organic phase was washed with brine, dried over Na₂SO₄, filtered, and concentrated. The crude product was purified by silica gel column (SiO₂, petroleum ether:EtOAc=50:1 to 30:1) to give tert-butyl (R)-4-(7-(4-chloropyridin-2-yl)-5-(N-methylformamido)-7H-pyrrolo[2,3-d]pyrimidin-4-yl)-2-methylpiperazine-1-carboxylate (60.0 mg, 59%) as a white solid. LC/MS ESI (m/z): 486 (M+H)⁺.

### Step 4. tert-Butyl (R)-4-(7-(4-chloropyridin-2-yl)-5-(dimethylamino)-7H-pyrrolo[2,3-d]pyrimidin-4-yl)-2-methylpiperazine-1-carboxylate

To a solution of tert-butyl (R)-4-(7-(4-chloropyridin-2-yl)-5-(N-methylformamido)-7H-pyrrolo[2,3-d]pyrimidin-4-yl)-2-methylpiperazine-1-carboxylate (50 mg, 0.10 mmol) in THF (5 mL) was added BH₃-THF (0.51 mL, 1.0M in THF) at 0° C. The mixture was stirred at 25° C. for 2 hrs and then quenched by addition of MeOH (5.0 m1). The mixture was concentrated, and the crude product was purified by silica gel column (SiO₂, petroleum ether:EtOAc=50:1 to 40:1) to give tert-butyl (R)-4-(7-(4-chloropyridin-2-yl)-5-(dimethylamino)-7H-pyrrolo[2,3-d]pyrimidin-4-yl)-2-methylpiperazine-1-carboxylate (30 mg, 64%) as a white solid. LC/MS ESI (m/z): 472 (M+H)⁺.

### Step 5. tert-Butyl (R)-4-(7-(4-cyanopyridin-2-yl)-5-(dimethylamino)-7H-pyrrolo[2,3-d]pyrimidin-4-yl)-2-methylpiperazine-1-carboxylate

To a solution of tert-butyl (R)-4-(7-(4-chloropyridin-2-yl)-5-(dimethylamino)-7H-pyrrolo[2,3-d]pyrimidin-4-yl)-2-methylpiperazine-1-carboxylate (30 mg, 0.064 mmol) in DMF (5 mL) was added Zn(CN)₂ (45 mg, 0.38 mmol) and Pd(PPh₃)₄ (37 mg, 0.032 mmol) at 25° C. The mixture was purged with N₂ 3 times. The mixture was heated to 120° C. and stirred for 3 hrs. The mixture was cooled to 25° C., poured into H₂O (100 mL), and extracted with EtOAc (50 mL×4). The combined organic phase was washed with brine, dried over Na₂SO₄, filtered, and concentrated. The product was purified by silica gel column (SiO₂, petroleum ether:EtOAc=50:1 to 30:1) to give tert-butyl (R)-4-(7-(4-cyanopyridin-2-yl)-5-(dimethyl amino)-7H-pyrrolo[2,3-d]pyrimidin-4-yl)-2-methylpiperazine-1-carboxylate (10 mg, 340%) as a yellow solid. LC/MS ESI (m/z): 463 (M+H)⁺. H NMR (400 MHz, CDCl₃) δ 9.36 (s, 1H), 8.58 (d, J=5.0 Hz, 1H), 8.43 (s, 1H), 7.58 (s, 1H), 7.30 (dd, J=5.0, 1.2 Hz, 1H), 4.81 (d, J=12.1 Hz, 1H), 4.45 (d, J=13.4 Hz, 1H), 4.38 (s, 1H), 3.93 (d, J=13.2 Hz, 1H), 3.44-3.322 (5, 1H), 3.26 (dd, J=13.2, 3.6 Hz, 1H), 2.99 (td, J=12.4, 3.2 Hz, 1H), 2.78 (s, 6H), 1.49 (s, 9H), 1.11 (d, J=6.8 Hz, 3H).

The following compounds were prepared by an analogous procedure to the synthesis of compound 427 from the corresponding acid anhydrides and the appropriate coupling partners/amines in different steps of the synthesis.

| Cmpd No. | Chemical Name | LCMS and ¹H NMR |
|---|---|---|
| 425 | tert-butyl (R)-4-(7-(4- | LC/MS ESI (m/z): 491 (M + H)⁺. 1H NMR (400 MHz, |
| | cyanopyridin-2-yl)-5- | CDCl₃) δ 9.38 (s, 1H), 8.56 (d, J = 5.0 Hz, 1H), 8.42 (s, |
| | (diethylamino)-7H-pyrrolo[2,3- | 1H), 7.61 (s, 1H), 7.28 (dd, J = 5.0, 1.1 Hz, 1H), 7.28 (dd, |
| | d]pyrimidin-4-yl)-2- | J = 5.0, 1.1 Hz, 1H), 4.87 (d, J = 12.7 Hz, 1H), 4.49 (d, J = |
| | methylpiperazine-1-carboxylate | 13.3 Hz, 1H), 4.35 (s, 1H), 3.90 (d, J = 13.3 Hz, 1H), 3.35 |
| | | (td, J = 13.0, 3.1 Hz, 1H), 3.29-3.18 (m, 3H), 3.18-3.06 |
| | | (m, 2H), 2.95 (td, J = 12.3, 3.3 Hz, 1H), 1.48 (s, 9H), 1.11 |
| | | (d, J = 6.8 Hz, 3H), 1.02 (s, 6H). |
| 441 | tert-butyl 4-(7-(4-cyanopyridin- | LC/MS ESI (m/z): 477 (M + H)⁺. ¹H NMR (400 MHz, |
| | 2-yl)-5-(diethylamino)-7H- | CDCl₃) δ 9.37 (s, 1H), 8.58 (d, J = 5.0 Hz, 1H), 8.45 (s, |
| | pyrrolo[2,3-d]pyrimidin-4- | 1H), 7.63 (s, 1H), 7.30 (dd, J = 5.0, 1.0 Hz, 1H), 3.85 (s, |
| | yl)piperazine-1-carboxylate | 4H), 3.61-3.56 (m, 4H), 3.19 (q, J = 7.0 Hz, 4H), 1.50 (s, |
| | | 9H), 1.03 (t, J = 7.0 Hz, 6H). |
| 430 | tert-butyl (R)-4-(7-(3- | LC/MS ESI (m/z): 471.6 (M + H)⁺. ¹H NMR (400 MHz, |
| | chlorophenyl)-5- | CDCl₃) δ 8.37 (s, 1H), 7.73 (s, 1H), 7.69-7.62 (m, 1H), |
| | (dimethylamino)-7H- | 7.47-7.37 (m, 1H), 7.30-7.27 (m, 1H), 6.73 (s, 1H), 4.88- |
| | pyrrolo[2,3-d]pyrimidin-4-yl)-2- | 4.79 (m, 1H), 4.45-4.35 (m, 2H), 3.99-3.89 (m, 1H), |
| | methylpiperazine-1-carboxylate | 3.48-3.33 (m, 1H), 3.30-3.16 (m, 1H), 3.04-2.91 (m, |
| | | 1H), 2.77 (s, 6H), 1.50 (s, 9H), 1.15 (d, J = 6.7 Hz, 3H). |

### Example 121. Synthesis of tert-butyl 4-(7-(4-cyanopyridin-2-yl)-5-cyclopropyl-7H-pyrrolo[2,3-d]pyrimidin-4-yl)-4,7-diazaspiro[2.5]octane-7-carboxylate (Compound 429)

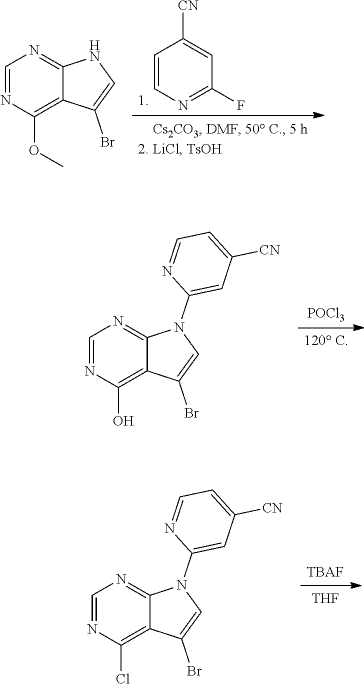

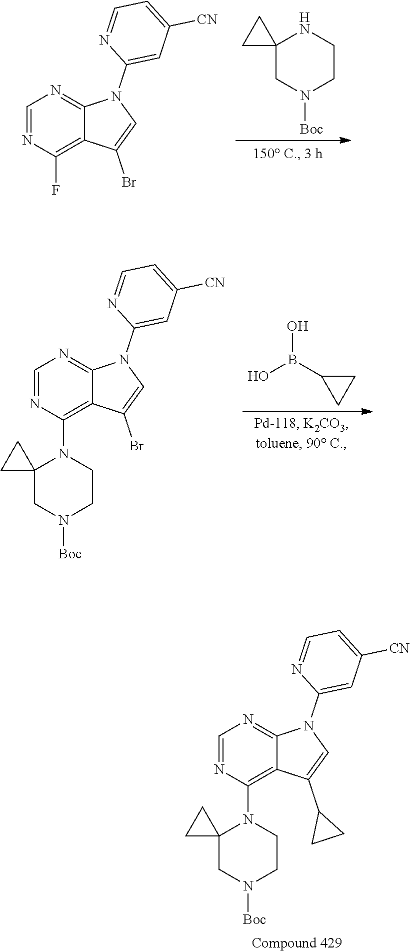

### Step 1. 2-(5-Bromo-4-methoxy-7H-pyrrolo[2,3-d]pyrimidin-7-yl)isonicotinonitrile

To a solution of 5-bromo-4-methoxy-7H-pyrrolo[2,3-d]pyrimidine (5.0 g, 22 mmol) in DMF (25 mL) were added 2-fluoroisonicotinonitrile (5.4 g, 44 mmol) and Cs₂CO₃ (36 g, 110 mmol). Then the resulting mixture was stirred at 50° C. for 5 h. The reaction was quenched with water, extracted with EtOAc twice, the combined organic layers were washed with brine, dried over Na₂SO₄, filtered, and concentrated. The residue was purified by flash column chromatography (silica gel, 0˜50%, ethyl acetate in petroleum ether) to afford 2-(5-bromo-4-methoxy-7H-pyrrolo[2,3-d]pyrimidin-7-yl)isonicotinonitrile (6.0 g, 83%) as a yellow solid. LC/MS ESI (m/z): 330, 332 (M+H)⁺.

### Step 2. 2-(5-Bromo-4-hydroxy-7H-pyrrolo[2,3-d]pyrimidin-7-yl)isonicotinonitrile

To a solution of 2-(5-bromo-4-methoxy-7H-pyrrolo[2,3-d]pyrimidin-7-yl)isonicotinonitrile (6.0 g, 18 mmol) in DMF (20 mL) were added 4-methylbenzenesulfonic acid (31 g, 180 mmol) and LiOH (7.7 g, 180 mmol). Then the resulting mixture was stirred at 110° C. for 2 h. The reaction was quenched with water, extracted with EtOAc twice, the combined organic layers were washed with brine, dried over Na₂SO₄, filtered and concentrated. The residue was purified by flash column chromatography (silica gel, 0˜50%, ethyl acetate in petroleum ether) to afford 2-(5-bromo-4-hydroxy-7H-pyrrolo[2,3-d]pyrimidin-7-yl)isonicotinonitrile (5.5 g, 96%) as a yellow solid. LC/MS ESI (m/z): 316, 318 (M+H)⁺.

### Step 3. 2-(5-Bromo-4-chloro-7H-pyrrolo[2,3-d]pyrimidin-7-yl)isonicotinonitrile

To a solution of POCl₃ (20 mL) was added 2-(5-bromo-4-hydroxy-7H-pyrrolo[2,3-d]pyrimidin-7-yl)isonicotinonitrile (5.5 g, 17 mmol). The resulting mixture was stirred at 120° C. under N₂ atmosphere overnight. The solvent was removed, and the residue poured into water and extracted with EtOAc twice. The combined organic layers were dried over Na₂SO₄, filtered, and concentrated. The residue was purified by flash chromatography (silica gel, 0-30% EtOAc in petroleum ether) to afford 2-(5-bromo-4-chloro-7H-pyrrolo[2,3-d]pyrimidin-7-yl) isonicotinonitrile (5.0 g, 86%) as a light yellow solid. LC/MS ESI (m/z): 334,336 (M+H)⁺.

### Step 4. 2-(5-Bromo-4-fluoro-7H-pyrrolo[2,3-d]pyrimidin-7-yl)isonicotinonitrile

To a solution of 2-(5-bromo-4-chloro-7H-pyrrolo[2,3-d]pyrimidin-7-yl)isonicotinonitrile (500 mg, 1.5 mmol) in THE (5 mL) were added TBAF (5.0 ml, 1.0M in THF) at 0° C. The resulting mixture was stirred at room temp overnight, then quenched with water, and extracted with EtOAc twice. The combined organic layers were dried over Na₂SO₄, filtered, and concentrated. The residue was purified by flash column chromatography (silica gel, 0-50%, ethyl acetate in petroleum ether) to afford 2-(5-bromo-4-fluoro-7H-pyrrolo[2,3-d]pyrimidin-7-yl)isonicotinonitrile (100 mg, 25%) as a light yellow solid. LC/MS ESI (m/z): 318,320 (M+H)⁺.

### Step 5. tert-Butyl 4-(5-bromo-7-(4-cyanopyridin-2-yl)-7H-pyrrolo[2,3-d]pyrimidin-4-yl)-4,7-diazaspiro[2.5]octane-7-carboxylate

To a solution of 2-(5-bromo-4-fluoro-7H-pyrrolo[2,3-d]pyrimidin-7-yl) isonicotinonitrile (100 mg, 031 mmol) was added tert-butyl 4,7-diazaspiro[2.5]octane-7-carboxylate (270 mg, 1.3 mmol). The resulting mixture was stirred at 150° C. under N₂ atmosphere for 3 hours. After cooling to room temperature, the mixture was diluted with EtOAc, filtered, and concentrated. The residue was purified by flash column chromatography (silica gel, 0˜50%, ethyl acetate in petroleum ether) to afford tert-butyl 4-(5-bromo-7-(4-cyanopyridin-2-yl)-7H-pyrrolo[2,3-d]pyrimidin-4-yl)-4,7-diazaspiro[2.5]octane-7-carboxylate (80 mg, 50%) as a yellow solid. LC/MS ESI (m/z): 510, 512 (M+H)⁺.

### Step 6. tert-Butyl 4-(7-(4-cyanopyridin-2-yl)-5-cyclopropyl-7H-pyrrolo[2,3-d]pyrimidin-4-yl)-4,7-diazaspiro[2.5]octane-7-carboxylate

To a solution of tert-butyl 4-(5-bromo-7-(4-cyanopyridin-2-yl)-7H-pyrrolo[2,3-d]pyrimidin-4-yl)-4,7-diazaspiro[2.5]octane-7-carboxylate (80 mg, 0.16 mmol) in toluene (5 mL) were added cyclopropylboronic acid (27 mg, 0.31 mmol), K₂CO₃ (430 mg, 3.1 mmol) and Pd-118 (4.0 mg, 0.020 mmol). The resulting mixture was heated to 90° C. overnight. After cooling to room temperature, the solvent was removed. The residue was purified by flash column chromatography (silica gel, 0˜50%, ethyl acetate in petroleum ether) to give the product (80 mg) which was further purified by prep-HPLC to afford tert-butyl 4-(7-(4-cyanopyridin-2-yl)-5-cyclopropyl-7H-pyrrolo[2,3-d]pyrimidin-4-yl)-4,7-diazaspiro[2.5]octane-7-carboxylate (35 mg, 40%) as a white solid. LC/MS ESI (m/z): 472 (M+H)⁺. ¹H NMR (400 MHz, CDCl₃) δ 9.25 (s, 1H), 8.54-8.48 (m, 2H), 7.69 (s, 1H), 7.28-7.26 (m, 1H), 3.91-3.79 (m, 2H), 3.69-3.58 (m, 2H), 3.49-3.21 (m, 2H), 1.92-1.84 (m, 1H), 1.41 (s, 9H), 0.97-0.88 (m, 4H), 0.75-0.64 (m, 4H).

The following compounds were prepared by an analogous procedures to the synthesis of compound 429 from the corresponding amine (tert-butyl 3,3-dimethylpiperazine-1-carboxylate)

| Cmpd No. | Chemical Name | LCMS and ¹H NMR |
|---|---|---|
| 421 | tert-butyl 4-(7-(4-cyanopyridin- | LC/MS ESI (m/z): 474 (M + H)⁺ ¹H NMR(400 MHz, |
| | 2-yl)-5-cyclopropyl-7H- | CD₃OD) δ 9.22 (s, 1H), 8.65 (d,J = 5.1 Hz, 1H), 8.57 (s, |
| | pyrrolo[2,3-d]pyrimidin-4-yl)- | 1H), 7.86 (s, 1H), 7.53 (d,J = 4.9 Hz, 1H), 3.73-3.64 (m, |
| | 3,3-dimethylpiperazine-1- | 2H), 3.61-3.55 (m, 2H), 3.43-3.37 (m, 2H), 2.29-2.21 |
| | carboxylate | (m, 1H), 1.55 (s, 6H), 1.50 (s, 9H), 1.09-1.03 (m, 2H), |
| | | 0.81-0.75 (m, 2H). |

### Example 122. tert-butyl (S)-4-(7-(6-cyanopyrimidin-4-yl)-5-cyclopropyl-7H-pyrrolo[2,3-d]pyrimidin-4-yl)-3-methylpiperazine-1-carboxylate (Compound 431)

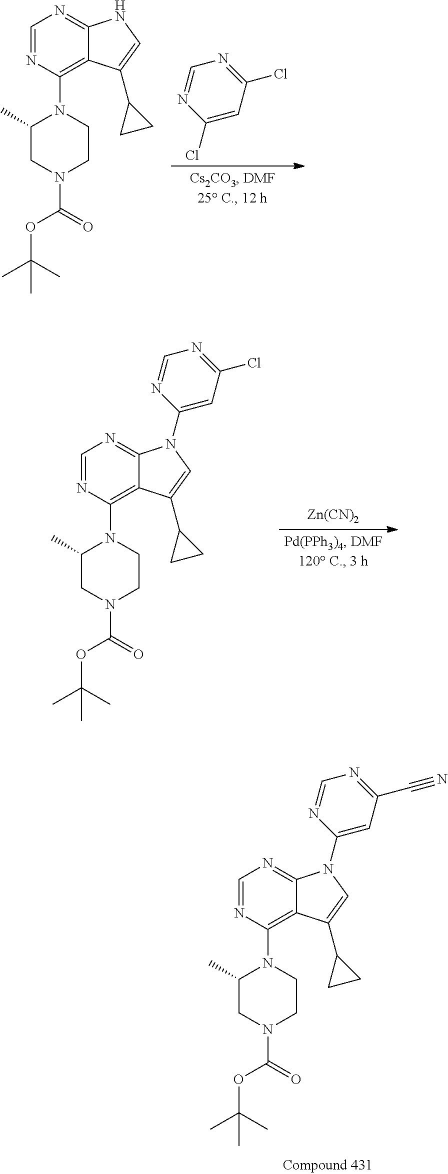

### Step 1. tert-Butyl (S)-4-(7-(6-chloropyrimidin-4-yl)-5-cyclopropyl-7H-pyrrolo[2,3-d]pyrimidin-4-yl)-3-methylpiperazine-1-carboxylate

To a solution of tert-butyl (3S)-4-{5-cyclopropyl-7H-pyrrolo[2,3-d]pyrimidin-4-yl}-3-methylpiperazine-1-carboxylate (100 mg, 0.28 mmol, prepared following the procedure outlined in compound 259) in DMF (5 mL) were added 4,6-dichloropyrimidine (0.051 mL, 0.56 mmol) and Cs₂CO₃ (180 mg, 0.56 mmol) at 25° C. The mixture was stirred at 25° C. for 12 hr, then poured into H₂O (500 mL) and extracted with EtOAc twice. The combined organic phases were washed with brine, dried over Na₂SO₄, filtered, and concentrated. The crude product was purified by silica gel column (SiO₂, petroleum ether:EtOAc=40:1 to 30:1) to give tert-butyl (S)-4-(7-(6-chloropyrimidin-4-yl)-5-cyclopropyl-7H-pyrrolo[2,3-d]pyrimidin-4-yl)-3-methylpiperazine-1-carboxylate (110 mg, 82%) as a yellow solid. LC/MS ESI (m/z): 470 (M+H)⁺.

### Step 2. tert-Butyl (S)-4-(7-(6-cyanopyrimidin-4-yl)-5-cyclopropyl-7H-pyrrolo[2,3-d]pyrimidin-4-yl)-3-methylpiperazine-1-carboxylate

To a solution of tert-butyl (S)-4-(7-(6-chloropyrimidin-4-yl)-5-cyclopropyl-7H-pyrrolo[2,3-d]pyrimidin-4-yl)-3-methylpiperazine-1-carboxylate (20 mg, 0.040 mmol) in DMF (5 mL) were added Zn(CN)₂ (28 mg, 0.24 mmol) and Pd(PPh₃)₄ (23 mg, 0.020 mmol) at 25° C. The mixture was degassed with N₂ 3 times followed by stirring at 120° C. for 3 hrs. The mixture was cooled to 25° C., poured into H₂O (100 mL), and extracted with EtOAc twice. The combined organic phases were washed with brine, dried over Na₂SO₄, filtered, and concentrated. The residue was purified by Prep-HPLC (0.1% formic acid). tert-butyl (S)-4-(7-(6-cyanopyrimidin-4-yl)-5-cyclopropyl-7H-pyrrolo[2,3-d]pyrimidin-4-yl)-3-methylpiperazine-1-carboxylate (11 mg, 55%) to provide as a yellow solid. LC/MS ESI (m/z): 461 (M+H)⁺. ¹H NMR (400 MHz, CDCl₃) δ 9.53 (d, J=1.0 Hz, 1H), 9.04 (d, J=0.9 Hz, 1H), 8.51 (s, 1H), 7.77 (s, 1H), 4.71 (s, 1H), 4.26-3.73 (m, 3H), 3.54 (t, J=11.4 Hz, 1H), 3.39-2.93 (m, 2H), 2.04-1.94 (m, 1H), 1.50 (s, 9H), 1.24 (d, J=6.4 Hz, 3H), 1.06 (d, J=8.1 Hz, 2H), 0.91-0.75 (m, 2H).

The following compound was prepared by an analogous procedure to the synthesis of compound 431 from the corresponding boronic acid.

| Cmpd No. | Chemical Name | LCMS and ¹H NMR |
|---|---|---|
| 437 | tert-butyl (S)-4-(7-(6- | LC/MS ESI (m/z): 515 (M + H)⁺. ¹H NMR (400 MHz, |
| | cyanopyrimidin-4-yl)-5-(2- | CDCl3) δ 9.64 (s, 1H), 9.10 (s, 1H), 8.57 (s, 1H), 8.28 (s, |
| | fluorophenyl)-7H-pyrrolo[2,3- | 1H), 7.51-7.35 (m, 2H), 7.27-7.17 (m, 2H), 4.45-4.04 |
| | d]pyrimidin-4-yl)-3- | (m, 1H), 3.90-3.63 (m, 1H), 3.56 (d, J = 13.1 Hz, 1H), |
| | methylpiperazine-1-carboxylate | 3.45 (d, J = 9.8 Hz, 1H), 3.06 (t, J = 11.4 Hz, 1H), 2.98- |
| | | 2.47 (m, 2H), 1.43 (s, 9H), 1.15-0.91 (m, 3H) |

### Example 123. tert-Butyl (S)-4-(7-(4-cyanopyrimidin-2-yl)-5-cyclopropyl-7H-pyrrolo[2,3-d]pyrimidin-4-yl)-3-methylpiperazine-1-carboxylate (Compound 432)

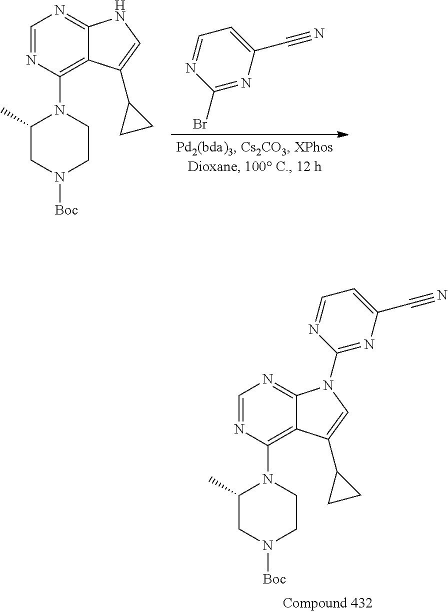 [SMILES: C[C@H]1CN(C(=O)OC(C)(C)C)CCN1c1ncnc2[nH]cc(C3CC3)c12 ; N#Cc1ccnc(Br)n1 ; "Pd2(bda)3, Cs2CO3, XPhosDioxane, 100° C., 12 h" ; C[C@H]1CN(C(=O)OC(C)(C)C)CCN1c1ncnc2c1c(C1CC1)cn2-c1nccc(C#N)n1]

### Step 1. tert-Butyl (S)-4-(7-(4-cyanopyrimidin-2-yl)-5-cyclopropyl-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (100 mg, 0.28 mmol, prepared following the procedure outlined in compound 259) in dioxane (5.0 mL) were added 2-bromopyrimidine-4-carbonitrile (77 mg, 0.42 mmol), Pd₂(dba)₃ (130 mg, 0.14 mmol), X-Phos (210 mg, 0.28 mmol) and Cs₂CO₃ (180 mg, 0.56 mmol) at 25° C. The mixture was degassed with N₂ 3 times. The mixture was stirred at 100° C. for 12 hrs. The mixture was cooled to 25° C., poured into H₂O (100 mL), and extracted with EtOAc twice. The combined organic phases were washed with brine, dried over Na₂SO₄, filtered, and concentrated under vacuo. The crude product was purified by Prep-HPLC (0.1% FA condition) to give tert-butyl (S)-4-(7-(4-cyanopyrimidin-2-yl)-5-cyclopropyl-7H-pyrrolo[2,3-d]pyrimidin-4-yl)-3-methylpiperazine-1-carboxylate (2.0 mg, 1.4%) as a yellow solid. LC/MS ESI (m/z): 461 (M+H)⁺. ¹H NMR (400 MHz, CDCl₃) δ 9.30 (d, J=5.9 Hz, 1H), 8.78 (d, J=5.8 Hz, 1H), 8.49 (s, 1H), 7.76 (s, 1H), 4.70 (s, 1H), 4.21-3.77 (m, 3H), 3.54 (t, J=11.5 Hz, 1H), 3.20 (d, J=77.9 Hz, 2H), 2.03-1.95 (m, 1H), 1.50 (s, 9H), 1.24 (d, J=6.7 Hz, 3H), 1.08 (d, J=8.1 Hz, 2H), 0.86 (d, J=27.0 Hz, 2H).

The following compound was prepared by the procedure similar to the synthesis of compound 432 from the corresponding aryl compound.

| Cmpd No. | Chemical Name | LCMS and ¹H NMR |
|---|---|---|
| 438 | tert-butyl (S)-4-(7-(4- | LC/MS ESI (m/z): 515 (M + H)⁺. ¹H NMR (400 MHz, |
| | cyanopyrimidin-2-yl)-5-(2- | CDCl₃) δ 9.17 (d, J = 4.8 Hz, 1H), 8.70 (s, 1H), 8.15 (s, |
| | fluorophenyl)-7H-pyrrolo[2,3- | 1H), 7.53 (d, J = 4.8 Hz, 1H), 7.47 (t, J = 7.0 Hz, 1H), 7.44- |
| | d]pyrimidin-4-yl)-3- | 7.36 (m, 1H), 7.26-7.19 (m, 2H), 4.30-4.01 (m, 1H), |
| | methylpiperazine-1-carboxylate | 3.90-3.61 (m, 1H), 3.52 (d, J = 13.1 Hz, 1H), 3.44 (d, J = |
| | | 13.1 Hz, 1H), 3.07 (t, J = 11.5 Hz, 1H), 2.91-2.48 (m, |
| | | 2H), 1.43 (s, 9H), 1.05-0.93 (m, 3H) |

### Example 124. Synthesis of tert-butyl 4-(7-(4-cyanopyridin-2-yl)-5-(2-fluorophenyl)-7H-pyrrolo[2,3-d]pyrimidin-4-yl)-4,7-diazaspiro[2.5]octane-7-carboxylate (Compound 435)

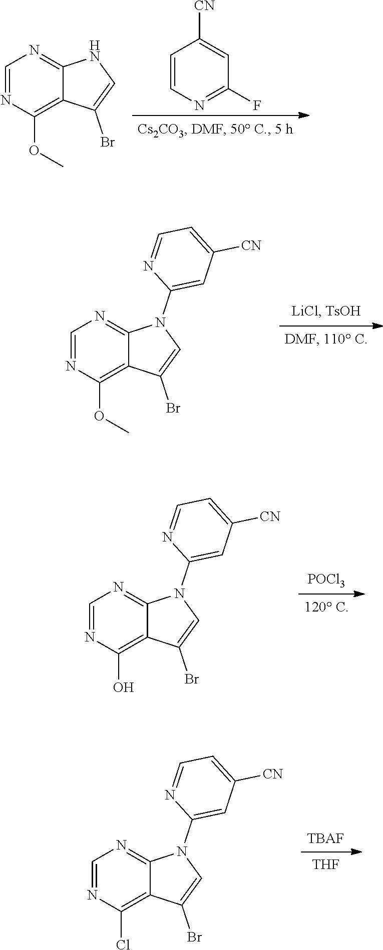

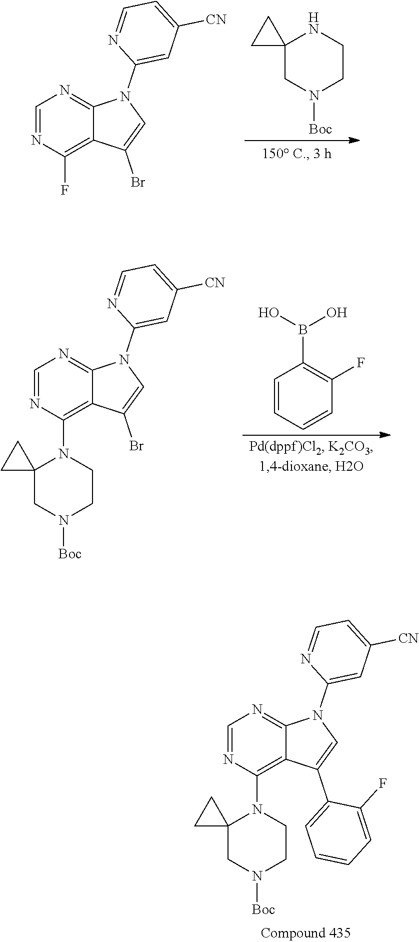

### Step 1. 2-(5-Bromo-4-methoxy-7H-pyrrolo[2,3-d]pyrimidin-7-yl)isonicotinonitrile

To a solution of 5-bromo-4-methoxy-7H-pyrrolo[2,3-d]pyrimidine (5.0 g, 22 mmol, prepared from MeONa and aryl bromide) in DMF (25 mL) were added 2-fluoroisonicotinonitrile (5.4 g, 44 mmol) and Cs₂CO₃ (36 g, 110 mmol). Then the resulting mixture was stirred at 50° C. for 5 h. The reaction was quenched with water, extracted with EtOAc twice, the combined organic layers were washed with brine, dried over Na₂SO₄, filtered and concentrated. The residue was purified by flash column chromatography (silica gel, 0˜50%, ethyl acetate in petroleum ether) to afford 2-(5-bromo-4-methoxy-7H-pyrrolo[2,3-d]pyrimidin-7-yl)isonicotinonitrile (6.0 g, 83%) as a yellow solid. LC/MS ESI (m/z): 330, 332 (M+H)⁺.

### Step 2. 2-(5-Bromo-4-hydroxy-7H-pyrrolo[2,3-d]pyrimidin-7-yl)isonicotinonitrile

To a solution of 2-(5-bromo-4-methoxy-7H-pyrrolo[2,3-d]pyrimidin-7-yl)isonicotinonitrile (6.0 g, 18 mmol) in DMF (20 mL) were added 4-methylbenzenesulfonic acid (31 g, 180 mmol) and LiOH (7.7 g, 180 mmol). Then the resulting mixture was stirred at 110° C. for 2 h. The reaction was quenched with water, extracted with EtOAc twice, the combined organic layers were washed with brine, dried over Na₂SO₄, filtered and concentrated. The residue was purified by flash column chromatography (silica gel, 0-50%, ethyl acetate in petroleum ether) to afford 2-(5-bromo-4-hydroxy-7H-pyrrolo[2,3-d]pyrimidin-7-yl)isonicotinonitrile (5.5 g, 96%) as a yellow solid. LC/MS ESI (m/z): 316, 318 (M+H)⁺.

### Step 3. 2-(5-Bromo-4-chloro-7H-pyrrolo[2,3-d]pyrimidin-7-yl)isonicotinonitrile

To a solution of POCl₃ (20 mL) was added 2-(5-bromo-4-hydroxy-7H-pyrrolo[2,3-d]pyrimidin-7-yl)isonicotinonitrile (5.5 g, 17 mmol). The resulting mixture was stirred at 120° C. under a N₂ atmosphere overnight. The solvent was removed, and the residue poured into water and extracted with EtOAc twice. The combined organic layers were dried over Na₂SO₄, filtered, and concentrated. The residue was purified by flash chromatography (silica gel, 0˜30% EtOAc in petroleum ether) to afford 2-(5-bromo-4-chloro-7H-pyrrolo[2,3-d]pyrimidin-7-yl)isonicotinonitrile (5.0 g, 86%) as a light yellow solid. LC/MS ESI (m/z): 334,336 (M+H)⁺.

### Step 4. 2-(5-Bromo-4-fluoro-7H-pyrrolo[2,3-d]pyrimidin-7-yl)isonicotinonitrile

To a solution of 2-(5-bromo-4-chloro-7H-pyrrolo[2,3-d]pyrimidin-7-yl)isonicotinonitrile (500 mg, 1.5 mmol) in THE (5 mL) were added TBAF (5.0 ml, 1.0M in THF) at 0° C. The resulting mixture was stirred at rt overnight. The reaction was quenched with water, extracted with EtOAc twice, the combined organic layers were dried over Na₂SO₄, filtered and concentrated. The residue was purified by flash column chromatography (silica gel, 0˜50%, ethyl acetate in petroleum ether) to afford 2-(5-bromo-4-fluoro-7H-pyrrolo[2,3-d]pyrimidin-7-yl)isonicotinonitrile (100 mg, 25%) as a light yellow solid. LC/MS ESI (m/z): 318, 320 (M+H)⁺.

### Step 5. tert-Butyl 4-(5-bromo-7-(4-cyanopyridin-2-yl)-7H-pyrrolo[2,3-d]pyrimidin-4-yl)-4,7-diazaspiro[2.5]octane-7-carboxylate

The mixture of 2-(5-bromo-4-fluoro-7H-pyrrolo[2,3-d]pyrimidin-7-yl) isonicotinonitrile (100 mg, 0.31 mmol) and tert-butyl 4,7-diazaspiro[2.5]octane-7-carboxylate (270 mg, 1.3 mmol) was stirred at 150° C. under N₂ atmosphere for 3 hours. After cooling to room temperature, the reaction was purified by flash column chromatography (silica gel, 0˜50%, ethyl acetate in petroleum ether) to afford tert-butyl 4-(5-bromo-7-(4-cyanopyridin-2-yl)-7H-pyrrolo[2,3-d]pyrimidin-4-yl)-4,7-diazaspiro[2.5]octane-7-carboxylate (80 mg, 50%) as a yellow solid. LC/MS ESI (m/z): 510, 512 (M+H)⁺.

### Step 6. tert-Butyl 4-(7-(4-cyanopyridin-2-yl)-5-(2-fluorophenyl)-7H-pyrrolo[2,3-d]pyrimidin-4-yl)-4,7-diazaspiro[2.5]octane-7-carboxylate

To a solution of tert-butyl 4-(5-bromo-7-(4-cyanopyridin-2-yl)-7H-pyrrolo[2,3-d]pyrimidin-4-yl)-4,7-diazaspiro[2.5]octane-7-carboxylate (80 mg, 0.16 mmol) in 1.4-dioxane (5 mL) and H₂O (1 m1) were added (2-fluorophenyl)boronic acid (44 mg, 0.31 mmol), K₂CO₃ (65 mg, 0.47 mmol) and Pd(dppf)Cl₂ (4.0 mg, 0.020 mmol). The resulting mixture was heated to 90° C. overnight. After being cooled down to room temperature, solvent was removed and the residue was purified by flash column chromatography (silica gel, 0˜50%, ethyl acetate in petroleum ether) to give product (100 mg). Further purification by prep-HPLC provided tert-butyl 4-(7-(4-cyanopyridin-2-yl)-5-(2-fluorophenyl)-7H-pyrrolo[2,3-d]pyrimidin-4-yl)-4,7-diazaspiro[2.5]octane-7-carboxylate (70 mg, 80%) as a white solid. LC/MS ESI (m/z): 526 (M+H)⁺. ¹H NMR (400 MHz, CDCl3) δ 9.33 (s, 1H), 8.60-8.54 (m, 2H), 8.23 (s, 1H), 7.35-7.27 (m, 3H), 7.15-7.09 (m, 2H), 3.55-3.37 (m, 2H), 3.32-2.94 (m, 2H), 2.93-2.82 (m, 2H), 1.34 (s, 9H), 0.89-0.76 (m, 4H).

The following compounds were prepared by analogous procedures to the synthesis of compound 435 from the corresponding amine (tert-butyl 3,3-dimethylpiperazine-1-carboxylate)

| Cmpd No. | Chemical Name | LCMS and ¹H NMR |
|---|---|---|
| 420 | tert-butyl 4-(7-(4-cyanopyridin- | LC/MS ESI (m/z): 528 (M + H)⁺ ¹H NMR(400 MHz, |
| | 2-yl)-5-(2-fluorophenyl)-7H- | MeOD) δ 9.31 (s, 1H), 8.70 (d,J = 5.0 Hz, 1H), 8.66 (s, |
| | pyrrolo[2,3-d]pyrimidin-4-yl)- | 1H), 8.30 (s, 1H), 7.61 (d,J = 5.0 Hz, 1H), 7.58-7.46 (m, |
| | 3,3-dimethylpiperazine-1- | 2H), 7.37-7.26 (m, 2H), 3.19-3.14 (m, 2H), 2.99-2.95 |
| | carboxylate | (m, 2H), 2.51-2.39 (m, 2H), 1.52 (s, 6H), 1.42 (s, 9H). |

### Example 125. Synthesis of 1,1,1-trifluoro-2-methylpropan-2-yl(S)-4-(7-(4-cyanopyridin-2-yl)-5-cyclobutyl-7H-pyrrolo[2,3-d]pyrimidin-4-yl)-3-methylpiperazine-1-carboxylate (Compound 436)

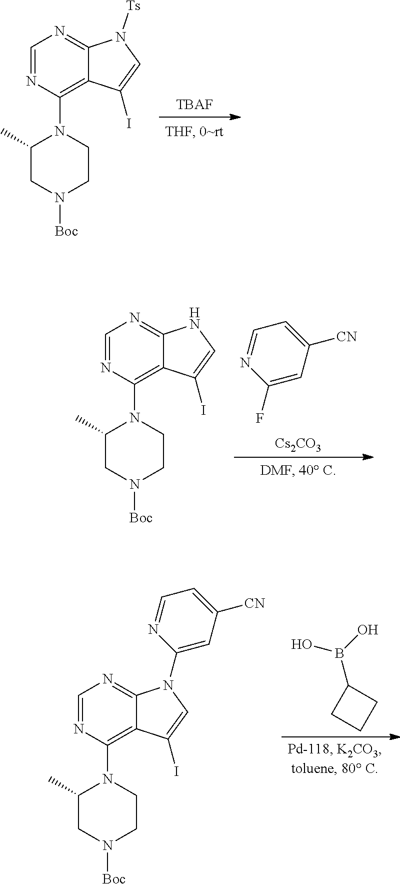

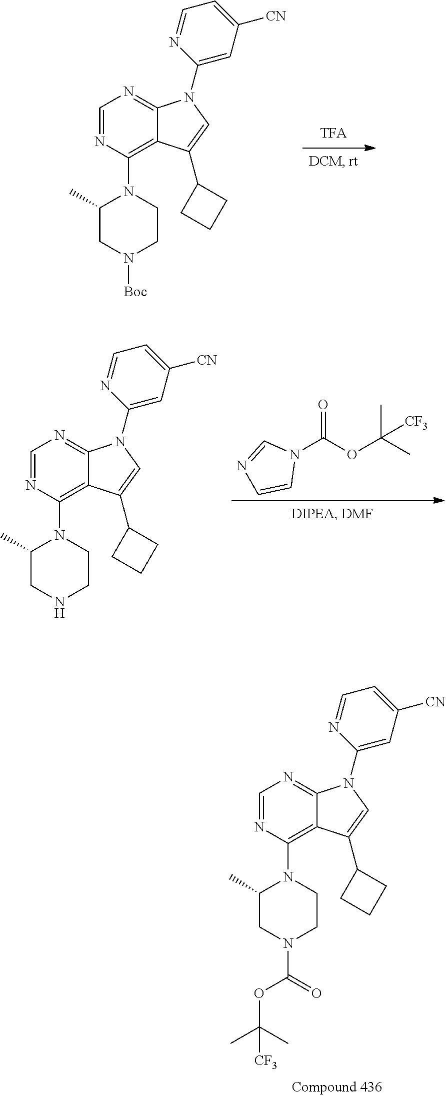

### Step 1. tert-Butyl (S)-4-(5-iodo-7H-pyrrolo[2,3-d]pyrimidin-4-yl)-3-methylpiperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (55 g, 110 mmol, prepared following the procedure outlined in compound 268) in THF (400 mL) was added TBAF (440 mL, 440 mmol, 1.0M in THF). The resulting mixture was stirred at 0° C. for 1.5 h. The reaction was quenched with water and extracted with EtOAc twice. The combined organic layers were washed with brine, dried over Na₂SO₄, filtered, and concentrated. The residue was purified by flash column chromatography (silica gel, 0˜10%, methanol in dichloromethane) to afford tert-butyl (S)-4-(5-iodo-7H-pyrrolo[2,3-d]pyrimidin-4-yl)-3-methylpiperazine-1-carboxylate (37 g, 91%) as a white solid. LC/MS ESI (m/z): 444 (M+H)⁺

### Step 2. tert-Butyl (S)-4-(7-(4-cyanopyridin-2-yl)-5-iodo-7H-pyrrolo[2,3-d]pyrimidin-4-yl)-3-methylpiperazine-1-carboxylate

To a solution of tert-butyl 4-{5-cyclopropyl-7H-pyrrolo[2,3-d]pyrimidin-4-yl}piperidine-1-carboxylate (4.5 g, 10 mmol) and 2-fluoropyridine-4-carbonitrile (1.8 g, 15 mmol) in DMF (60 mL) was added Cs₂CO₃ (16 g, 50 mmol) and the resulting mixture was stirred at 40° C. for 18 h. After being cooled down to room temperature, the reaction mixture was filtered. The filtrate was diluted with ice water and extracted with EtOAc twice. The combined organic layers were washed with brine, dried over Na₂SO₄, filtered, and concentrated. The residue was purified by column chromatography on silica gel (0˜80% ethyl acetate in petroleum ether) to give tert-butyl (S)-4-(7-(4-cyanopyridin-2-yl)-5-iodo-7H-pyrrolo[2,3-d]pyrimidin-4-yl)-3-methylpiperazine-1-carboxylate (3.6, 66%). LC/MS(ESI) m/z: 546 (M+H)⁺

### Step 3. tert-Butyl(S)-4-(7-(4-cyanopyridin-2-yl)-5-cyclobutyl-7H-pyrrolo[2,3-d]pyrimidin-4-yl)-3-methylpiperazine-1-carboxylate

A suspension of tert-butyl (3S)-4-[7-(4-cyanopyridin-2-yl)-5-iodo-7H-pyrrolo[2,3-d]pyrimidin-4-yl]-3-methylpiperazine-1-carboxylate (550 mg, 1.0 mmol), cyclobutylboronic acid (0.19 mL, 2.0 mmol), K₂CO₃ (2.7 g, 19 mmol) and Pd-118 (66 mg, 0.10 mmol) in toluene (50 mL) was stirred at 80° C. under a N₂ atmosphere for 18 h. After being cooled down to room temperature the solvent was removed. The residue was purified by flash column chromatography on silica gel (0˜80% ethyl acetate in petroleum ether) and further purified by prep-HPLC to give tert-butyl (S)-4-(7-(4-cyanopyridin-2-yl)-5-cyclobutyl-7H-pyrrolo[2,3-d]pyrimidin-4-yl)-3-methylpiperazine-1-carboxylate (50 mg, 10%). LC/MS (ESI) (m/z): 474 (M+H)⁺. ¹H NMR (400 MHz, CDCl3) δ 9.33 (s, 1H), 8.66-8.58 (m, 1H), 8.54 (s, 1H), 8.12 (s, 1H), 7.40-7.32 (m, 1H), 4.36-4.17 (m, 1H), 4.07-3.89 (m, 1H), 3.80-3.63 (m, 2H), 3.58-3.43 (m, 3H), 3.29-3.12 (m, 1H), 2.53-2.38 (m, 2H), 2.29-2.02 (m, 3H), 2.00-1.92 (m, 1H), 1.50 (s, 9H), 1.20 (d, J=4.8 Hz, 3H). This reaction was repeated to make another batch for the next step.

### Step 4. (S)-2-(5-Cyclobutyl-4-(2-methylpiperazin-1-yl)-7H-pyrrolo[2,3-d]pyrimidin-7-yl)isonicotinonitrile

At 0° C., to a solution of tert-butyl (S)-4-(7-(4-cyanopyridin-2-yl)-5-cyclobutyl-7H-pyrrolo[2,3-d]pyrimidin-4-yl)-3-methylpiperazine-1-carboxylate (100 mg, 0.21 mmol) in DCM (2 mL) was added TFA (0.31 mL, 4.2 mmol) and the reaction was stirred for 3 h. After removal of solvent, the residue used directly in next step without further purification. LC/MS(ESI) m/z: 374 (M+H)⁺

### Step 5. 1,1,1-Trifluoro-2-methylpropan-2-yl (S)-4-(7-(4-cyanopyridin-2-yl)-5-cyclobutyl-7H-pyrrolo[2,3-d]pyrimidin-4-yl)-3-methylpiperazine-1-carboxylate

To a solution of 2-{5-cyclobutyl-4-[(2S)-2-methylpiperazin-1-yl]-7H-pyrrolo[2,3-d]pyrimidin-7-yl}pyridine-4-carbonitrile (35 mg, 0.090 mmol) and DIEA (0.090 mL, 0.56 mmol) in DMF (2 mL) was added a solution of 1,1,1-trifluoro-2-methylpropan-2-yl 1H-imidazole-1-carboxylate (42 mg, 0.19 mmol, prepared following the procedure outlined in compound 243). The resulting mixture was stirred at 75° C. for 18 h. After being cooled down to room temperature, the reaction mixture was partitioned between EtOAc, and water and the organic layer was separated. The aqueous layer was extracted with EtOAc twice and the combined organic layers dried over anhydrous Na₂SO₄, filtered, and concentrated. The residue was purified by column chromatography on silica gel and prep-HPLC to give 1,1,1-trifluoro-2-methylpropan-2-yl(S)-4-(7-(4-cyanopyridin-2-yl)-5-cyclobutyl-7H-pyrrolo[2,3-d]pyrimidin-4-yl)-3-methylpiperazine-1-carboxylate (7.5 mg, 15%) as a white solid. LC/MS (ESI) (m/z): 528 (M+H)^{+ 1}H NMR (400 MHz, CDCl₃) δ 9.34 (s, 1H), 8.61 (d, J=5.0 Hz, 1H), 8.52 (s, 1H), 8.12 (s, 1H), 7.39-7.31 (m, 1H), 4.24 (s, 1H), 3.96 (dd, J=65.1, 13.4 Hz, 1H), 3.71 (dd, J=20.3, 11.4 Hz, 2H), 3.51 (d, J=12.6 Hz, 3H), 3.37-3.14 (m, 1H), 2.45 (ddd, J=14.5, 9.9, 4.9 Hz, 2H), 2.29-1.91 (m, 4H), 1.72 (d, J=3.9 Hz, 6H), 1.19 (d, J=6.2 Hz, 3H).

### Example 126. Synthesis of tert-butyl (S)-4-(7-(5-cyano-1H-imidazol-2-yl)-5-cyclopropyl-7H-pyrrolo[2,3-d]pyrimidin-4-yl)-3-methylpiperazine-1-carboxylate (Compound 440)

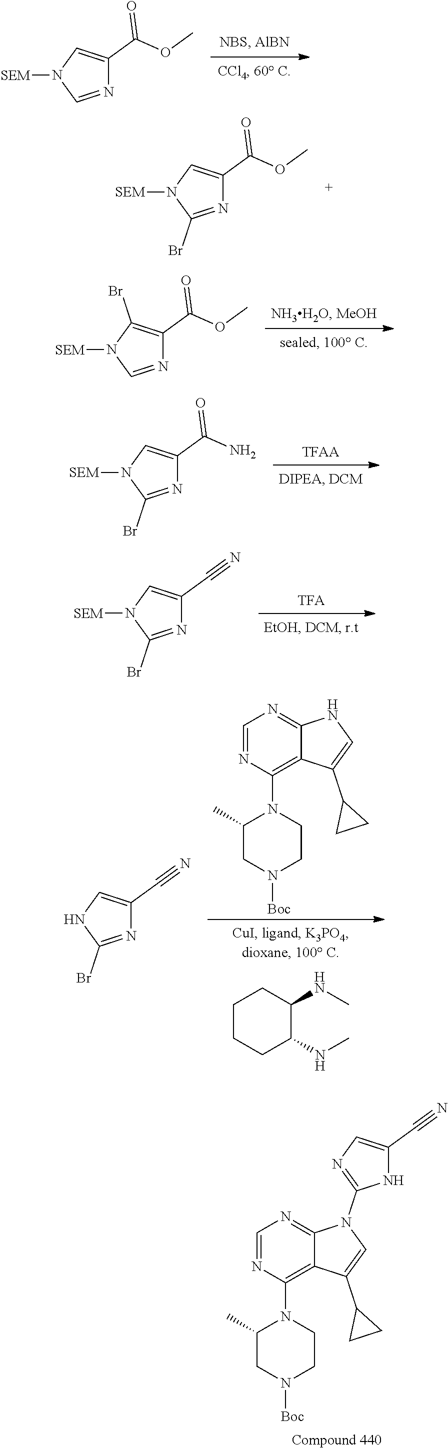

### Step 1. Methyl 2-bromo-1-((2-(trimethylsilyl)ethoxy)methyl)-1H-imidazole-4-carboxylate

To a solution of methyl 1-((2-(trimethylsilyl)ethoxy)methyl)-1H-imidazole-4-carboxylate (260 mg, 1.0 mmol) in carbon tetrachloride (10 mL) was added NBS (200 mg, 1.1 mmol) and AIBN (16 mg, 0.10 mmol). The reaction was heated at 60° C. for 3 hours then diluted with sat. NaHCO₃ and the layers were separated. The organics were concentrated and purified by flash column chromatography (silica gel, 0-60% ethyl acetate in petroleum ether) to afford methyl 2-bromo-1-((2-(trimethylsilyl)ethoxy)methyl)-1H-imidazole-4-carboxylate as a white solid (200 mg, 61% yield). LC/MS ESI (m/z): 335/337 (Br) (M+H)⁺. ¹H NMR (400 MHz, CDCl₃) δ 7.77 (s, 1H), 5.30 (s, 2H), 3.90 (s, 3H), 3.59-3.51 (m, 2H), 0.96-0.89 (m, 2H), −0.01 (s, 9H).

Methyl 5-bromo-1-((2-(trimethylsilyl)ethoxy)methyl)-1H-imidazole-4-carboxylate was also obtained as colorless oil (64 mg, 19%). LC/MS ESI (m/z) 335/337 (Br) (M+H)⁺. ¹H NMR (400 MHz, CDCl₃) δ 7.69 (s, 1H), 5.74 (s, 2H), 3.86 (s, 3H), 3.63-3.56 (m, 2H), 0.94-0.88 (m, 2H), −0.03 (s, 9H).

### Step 2. 2-Bromo-1-((2-(trimethylsilyl)ethoxy)methyl)-1H-imidazole-4-carboxamide

Methyl 2-bromo-1-((2-(trimethylsilyl)ethoxy)methyl)-1H-imidazole-4-carboxylate (1.3 g, 3.9 mmol) was dissolved in MeOH (2 mL) and NH₃/H₂O (4 mL) and the reaction mixture was heated at 100° C. for 2 h. The reaction was diluted with water and the layers were separated. The organics were concentrated to afford 2-bromo-1-((2-(trimethylsilyl)ethoxy)methyl)-1H-imidazole-4-carboxamide as a white solid (950 mg, 77% yield). LC/MS ESI (m/z): 320/322 (Br) (M+H)⁺.

### Step 3. 2-Bromo-1-((2-(trimethylsilyl)ethoxy)methyl)-1H-imidazole-4-carbonitrile

To a solution of 2-bromo-1-((2-(trimethylsilyl)ethoxy)methyl)-1H-imidazole-4-carboxamide (900 mg, 2.8 mmol) was in DCM (20 mL) and DIPEA (7 mL) was added TFAA (2 mL) and the mixture was stirred at 0° C. for 3 h. The reaction was diluted with brine and the layers were separated. The organics were concentrated to afford 2-bromo-1-((2-(trimethylsilyl)ethoxy)methyl)-1H-imidazole-4-carbonitrile as a yellow oil that was carried forward to the next step without further purification. LC/MS ESI (m/z): 302/304 (Br) (M+H)⁺.

### Step 4. 2-Bromo-1H-imidazole-5-carbonitrile

To a mixture of 2-bromo-1-((2-(trimethylsilyl)ethoxy) methyl)-1H-imidazole-4-carbonitrile (obtained from the previous step) was in DCM (5 mL) and EtOH (0.5 mL) was added TFA (10 mL). The mixture was stirred at 0° C. for 3 h, then diluted with brine and layers were separated. The organics were concentrated, and the residue was purified by flash column chromatography to afford 2-bromo-1H-imidazole-5-carbonitrile as a white solid (400 mg, 83%, over two steps). LC/MS ESI (m/z): 172/174 (Br) (M+H)⁺. ¹H NMR (400 MHz, MeOD) δ 7.90 (s, 1H), 2.67 (s, 1H).

### Step 5. tert-Butyl (S)-4-(7-(5-cyano-1H-imidazol-2-yl)-5-cyclopropyl-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (71 mg, 0.20 mmol, prepared following the procedure of compound 260, step 4) and 2-bromothiazole-5-carbonitrile (34 mg, 0.20 mmol) in dioxane (1 mL) were added CuI (19 mg, 0.10 mmol), K₃PO₄ (210 mg, 1.0 mmol) and trans-N,N′-dimethylcyclohexane-1,2-diamine (14 mg, 0.10 mmol). The resulting mixture was stirred at 100° C. under N₂ for 2 h. After being cooled to room temperature, the reaction was partitioned between EtOAc and water and the organic layer was separated. The aqueous layer was extracted with EtOAc twice and the combined organic layers were washed with brine, filtered, and concentrated. The residue was purified by flash column chromatography (silica gel, 0˜10%, MeOH in DCM) to give copper complexes with product as a purple solid. The complexes were partitioned between DCM and EDTANa₂ (aqueous) until organic phase was colorless. The organic layer was concentrated and further purified by prep-HPLC to afford tert-butyl (S)-4-(7-(5-cyano-1H-imidazol-2-yl)-5-cyclopropyl-7H-pyrrolo[2,3-d]pyrimidin-4-yl)-3-methylpiperazine-1-carboxylate (6.2 mg, 14% yield) as a white solid. LC/MS ESI (m/z): 449 (M+H)⁺. ¹H NMR (400 MHz, CDCl₃) δ 12.26 (br, 1H), 8.39 (s, 1H), 7.54 (s, 1H), 7.48 (s, 1H), 5.00-4.70 (m, 1H), 4.34-3.72 (m, 3H), 3.53 (t, J=11.3 Hz, 1H), 3.36-2.88 (m, 2H), 1.96-1.89 (m, 1H), 1.49 (s, 9H), 1.26 (d, J=6.7 Hz, 3H), 1.09-0.98 (m, 2H), 0.86-0.75 (m, 2H).

### Example 127. Synthesis of tert-butyl (S)-4-(7-(4-cyanopyridin-2-yl)-5-(cyclopropyl(methyl)amino)-7H-pyrrolo[2,3-d]pyrimidin-4-yl)-3-methylpiperazine-1-carboxylate (Compound 442)

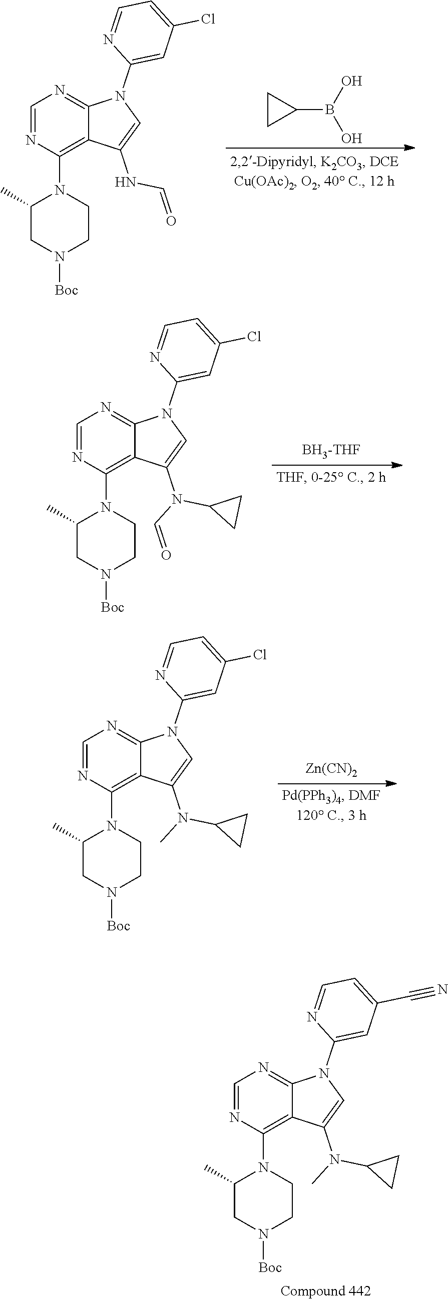

### Step 1. tert-Butyl (S)-4-(7-(4-chloropyridin-2-yl)-5-(N-cyclopropylformamido)-7H-pyrrolo[2,3-d]pyrimidin-4-yl)-3-methylpiperazine-1-carboxylate

To a solution of tert-butyl (S)-4-(7-(4-chloropyridin-2-yl)-5-formamido-7H-pyrrolo[2,3-d]pyrimidin-4-yl)-3-methylpiperazine-1-carboxylate (100 mg, 0.21 mmol, prepared following the procedure outlined in compound 427) in DCE (40 mL) were added cyclopropylboronic acid (36 mg, 0.42 mmol), K₂CO₃ (88 mg, 0.64 mmol), 2,2′-dipyridyl (330 mg, 0.21 mmol) and Cu(OAc)₂ (38 mg, 0.21 mmol) at 25° C. The mixture was degassed with O₂ 3 times and stirred at 40° C. under O₂ overnight. The mixture was cooled to 25° C., then poured into H₂O (100 mL) and extracted with EtOAc twice. The combined organic phases were washed with brine, dried over Na₂SO₄, filtered, and concentrated. The residue was purified by silica gel column (SiO₂, petroleum ether:EtOAc=50:1 to 30:1) to provide tert-butyl (S)-4-(7-(4-chloropyridin-2-yl)-5-(N-cyclopropylformamido)-7H-pyrrolo[2,3-d]pyrimidin-4-yl)-3-methylpiperazine-1-carboxylate (50 mg, 47%) as a white solid. LC/MS ESI (m/z): 512 (M+H)⁺.

### Step 2. tert-Butyl (S)-4-(7-(4-chloropyridin-2-yl)-5-(cyclopropyl(methyl)amino)-7H-pyrrolo[2,3-d]pyrimidin-4-yl)-3-methylpiperazine-1-carboxylate

To a solution of tert-butyl (S)-4-(7-(4-chloropyridin-2-yl)-5-(N-cyclopropylformamido)-7H-pyrrolo[2,3-d]pyrimidin-4-yl)-3-methylpiperazine-1-carboxylate (50 mg, 0.098 mmol) in THE (4 mL) was added BH₃-THF (0.98 mL, 1.0M in THF) at 0° C. The mixture was warmed to 25° C. and stirred for 2 hrs. The mixture was quenched by addition of MeOH (5 mL) at 0° C. and concentrated. The residue was purified by silica gel column (SiO₂, petroleum ether:EtOAc=50:1 to 40:1) to provide tert-butyl (S)-4-(7-(4-chloropyridin-2-yl)-5-(cyclopropyl(methyl)amino)-7H-pyrrolo[2,3-d]pyrimidin-4-yl)-3-methylpiperazine-1-carboxylate (20 mg, 41%) as a white solid. LC/MS ESI (m/z): 498 (M+H)⁺.

### Step 3. tert-Butyl (S)-4-(7-(4-cyanopyridin-2-yl)-5-(cyclopropyl(methyl)amino)-7H-pyrrolo[2,3-d]pyrimidin-4-yl)-3-methylpiperazine-1-carboxylate

To a solution of tert-butyl (S)-4-(7-(4-chloropyridin-2-yl)-5-(cyclopropyl(methyl)amino)-7H-pyrrolo[2,3-d]pyrimidin-4-yl)-3-methylpiperazine-1-carboxylate (20 mg, 0.040 mmol) in DMF (5 mL) were added Zn(CN)₂ (28 mg, 0.24 mmol) and Pd(PPh₃)₄ (23 mg, 0.020 mmol) at 25° C. The mixture was purged with N₂ 3 times. The mixture was stirred at 120° C. for 3 hrs then cooled to 25° C. The mixture was poured into H₂O (20 mL) and extracted with EtOAc twice. The combined organic phase was washed with brine, dried over Na₂SO₄, filtered, and concentrated. The residue was purified by Prep-HPLC (0.1% formic acid) to provide tert-butyl (S)-4-(7-(4-cyanopyridin-2-yl)-5-(cyclopropyl(methyl)amino)-7H-pyrrolo[2,3-d]pyrimidin-4-yl)-3-methylpiperazine-1-carboxylate (11 mg, 55%) as a yellow solid. LC/MS ESI (m/z): 489 (M+H)⁺. ¹H NMR (400 MHz, CDCl₃) δ 9.30-9.24 (m, 1H), 8.52 (d, J=5.0 Hz, 1H), 8.36 (s, 1H), 7.73 (s, 1H), 7.24 (dd, J=5.0, 1.2 Hz, 1H), 4.78 (s, 1H), 4.19-3.67 (m, 3H), 3.28 (t, J=11.7 Hz, 1H), 3.22-2.83 (m, 2H), 2.77 (s, 3H), 2.27-2.19 (m, 1H), 1.42 (s, 9H), 1.05 (s, 3H), 0.72 (d, J=6.4 Hz, 2H), 0.51-0.37 (m, 2H).

### Example 128. Synthesis of tert-butyl (S)-4-(7-(4-cyanopyridin-2-yl)-5-(isopropyl(methyl)amino)-7H-pyrrolo[2,3-d]pyrimidin-4-yl)-3-methylpiperazine-1-carboxylate (Compound 443)

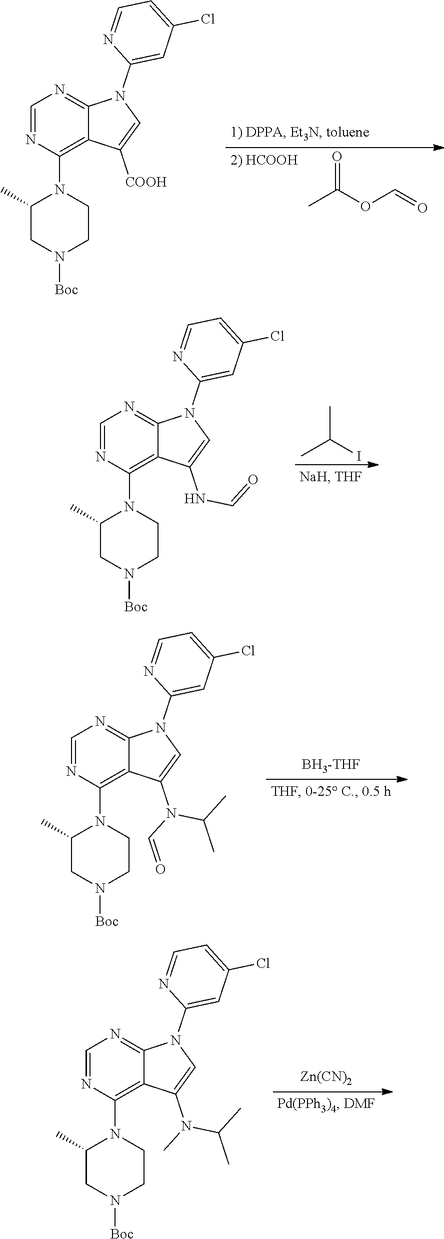

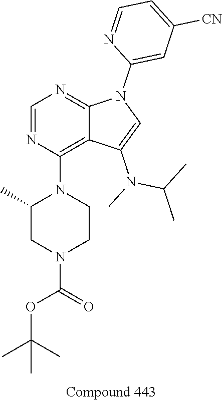 [SMILES: CC(C)N(C)c1cn(-c2cc(C#N)ccn2)c2ncnc(N3CCN(C(=O)OC(C)(C)C)C[C@@H]3C)c12]

### Step 1. tert-Butyl (S)-4-(7-(4-chloropyridin-2-yl)-5-formamido-7H-pyrrolo[2,3-d]pyrimidin-4-yl)-3-methylpiperazine-1-carboxylate

To a solution of (S)-4-(4-(tert-butoxycarbonyl)-2-methylpiperazin-1-yl)-7-(4-chloropyridin-2-yl)-7H-pyrrolo[2,3-d]pyrimidine-5-carboxylic acid (200 mg, 0.42 mmol, prepared following the procedure outlined in compound 366) in toluene (5 mL) were added TEA (0.20 mL, 1.3 mmol) and DPPA (230 mg, 0.84 mmol). The resulting mixture was stirred at room temperature for 2 hr. A second mixture of HCOOH (0.75 mL) and acetic formic anhydride (2.0 mL) was stirred at 60° C. for 2 hr to afford the mixed anhydride. The mixed anhydride was added into the first reaction and the resulting mixture was heated to 60° C. for 2 hr. After being cooled down to room temperature, the reaction was quenched with NaHCO₃ and extracted with EtOAc twice. The combined organic layers were washed with brine, dried over Na₂SO₄, filtered, and concentrated. The residue was purified by flash column chromatography (silica gel, ethyl acetate in petroleum ether) to afford tert-butyl (S)-4-(7-(4-chloropyridin-2-yl)-5-formamido-7H-pyrrolo[2,3-d]pyrimidin-4-yl)-3-methylpiperazine-1-carboxylate (170 mg, 83%) as a light yellow solid. LC/MS ESI (m/z): 472 (M+H)⁺.

### Step 2. tert-Butyl (S)-4-(7-(4-chloropyridin-2-yl)-5-(N-isopropylformamido)-7H-pyrrolo[2,3-d]pyrimidin-4-yl)-3-methylpiperazine-1-carboxylate

At 0° C., to a solution of NaH (27 mg, 0.68 mmol) in THE (5 mL) was added tert-butyl (S)-4-(7-(4-chloropyridin-2-yl)-5-formamido-7H-pyrrolo[2,3-d]pyrimidin-4-yl)-3-methylpiperazine-1-carboxylate (160 mg, 0.34 mmol) and the mixture stirred at 0° C. for 10 min. 2-Iodopropane (86 mg, 0.50 mmol) was added, and the resulting mixture was heated to 50° C. overnight under N₂. After being cooled down to room temperature, the reaction was quenched with ice water and extracted with EtOAc twice. The combined organic layers were washed with brine, dried over Na₂SO₄, filtered, and concentrated. The residue was purified by flash column chromatography (silica gel, ethyl acetate in petroleum ether) to afford tert-butyl (S)-4-(7-(4-chloropyridin-2-yl)-5-(N-isopropylformamido)-7H-pyrrolo[2,3-d]pyrimidin-4-yl)-3-methylpiperazine-1-carboxylate (130 mg, 74%) as a light yellow solid. LC/MS ESI (m/z): 514 (M+H)⁺.

### Step 3. tert-Butyl (S)-4-(7-(4-chloropyridin-2-yl)-5-(isopropyl(methyl)amino)-7H-pyrrolo[2,3-d]pyrimidin-4-yl)-3-methylpiperazine-1-carboxylate

At 0° C., to a solution of tert-butyl (S)-4-(7-(4-chloropyridin-2-yl)-5-(N-isopropylformamido)-7H-pyrrolo[2,3-d]pyrimidin-4-yl)-3-methylpiperazine-1-carboxylate (130 mg, 0.25 mmol) in THE (2 mL) was added BH₃-THF (1.0 mL, 1.0 M). After stirring at room temperature for 1 hr, the reaction was quenched with ice water and extracted with EtOAc twice. The combined organic layers were washed with brine, dried over Na₂SO₄, filtered, and concentrated. The residue was purified by flash column chromatography (silica gel, ethyl acetate in petroleum ether) to afford tert-butyl (S)-4-(7-(4-chloropyridin-2-yl)-5-(isopropyl(methyl)amino)-7H-pyrrolo[2,3-d]pyrimidin-4-yl)-3-methylpiperazine-1-carboxylate (72 mg, 57%) as a colorless oil. LC/MS ESI (m/z): 500 (M+H)⁺.

### Step 4. tert-Butyl (S)-4-(7-(4-cyanopyridin-2-yl)-5-(isopropyl(methyl)amino)-7H-pyrrolo[2,3-d]pyrimidin-4-yl)-3-methylpiperazine-1-carboxylate

To the solution of tert-butyl (S)-4-(7-(4-chloropyridin-2-yl)-5-(isopropyl(methyl)amino)-7H-pyrrolo[2,3-d]pyrimidin-4-yl)-3-methylpiperazine-1-carboxylate (72 mg, 0.14 mmol) in DMF (5 mL) were added Zn(CN)₂ (98 mg, 0.84 mmol) and Pd(PPh₃)₄ (80 mg, 0.070 mmol) respectively. The resulting reaction mixture was stirred at 120° C. under N₂ overnight. After being cooled down to r.t., the reaction mixture was concentrated under reduced pressure. The residue was purified by flash column chromatography (silica gel, 0-30%, ethyl acetate in petroleum ether) and prep-HPLC (C18, MeCN in H₂O) to afford tert-butyl (S)-4-(7-(4-cyanopyridin-2-yl)-5-(isopropyl(methyl)amino)-7H-pyrrolo[2,3-d]pyrimidin-4-yl)-3-methylpiperazine-1-carboxylate (19 mg, 27%) as a yellow solid. LC/MS ESI (m/z): 491 (M+H)⁺. ¹H NMR (400 MHz, CDCl 3) δ 9.38 (s, 1H), 8.58 (d, J=5.0 Hz, 1H), 8.42 (s, 1H), 7.61 (s, 1H), 7.29 (dd, J=5.0, 1.0 Hz, 1H), 5.09 (s, 1H), 4.35-3.85 (m, 3H), 3.72-3.64 (m, 1H), 3.47-3.27 (m, 2H), 3.03-2.83 (m, 1H), 2.66 (s, 3H), 1.49 (s, 9H), 1.22 (d, J=6.5 Hz, 3H), 1.16 (d, J=5.3 Hz, 3H), 0.95 (d, J=6.4 Hz, 3H).

### Example 129. Synthesis of tert-butyl (S)-4-(7-(5-cyano-1-methyl-1H-pyrrol-3-yl)-5-cyclopropyl-7H-pyrrolo[2,3-d]pyrimidin-4-yl)-3-methylpiperazine-1-carboxylate (Compound 445)

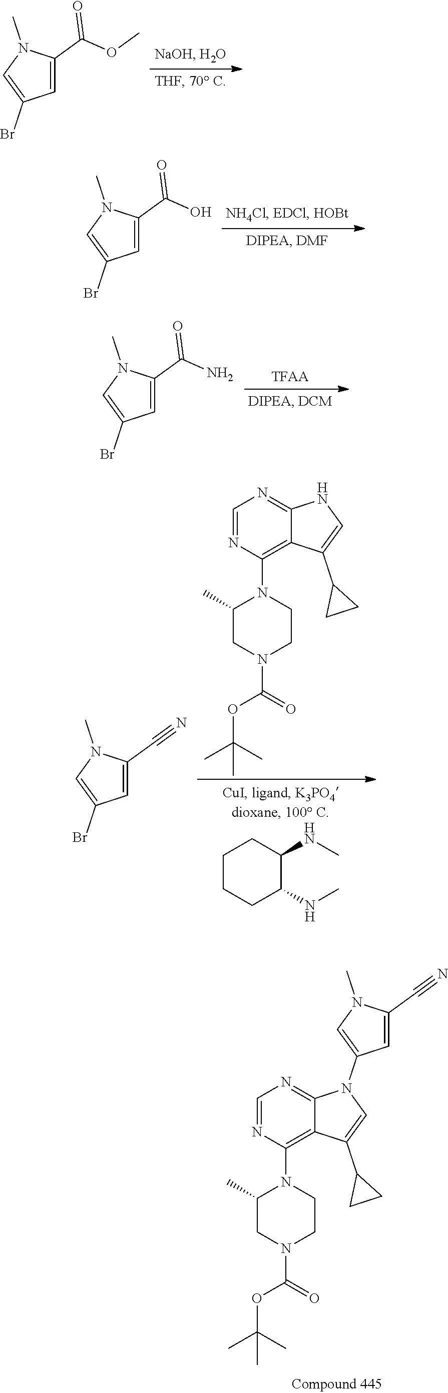

### Step 1. 4-Bromo-1-methyl-1H-pyrrole-2-carboxylic acid

A solution of Methyl 4-bromo-1-methyl-1H-pyrrole-2-carboxylate (440 mg, 2.0 mmol) in H₂O (2 mL) and THF (2 mL) was added to NaOH (320 mg, 8.0 mmol) and the reaction mixture was heated at 70° C. for 4 h. The reaction was quenched with HCl (1.0 M), diluted with water, and the layers were separated. The organics were concentrated to afford 4-bromo-1-methyl-1H-pyrrole-2-carboxylic acid as a white solid (390 mg, 96%). LC/MS ESI (m/z): 202/204 (Br) (M−H)⁺.

### Step 2. 4-Bromo-1-methyl-1H-pyrrole-2-carboxamide

To a solution of 4-bromo-1-methyl-1H-pyrrole-2-carboxylic acid (450 mg, 2.2 mmol) in DMF (10 mL) and DIPEA (3 mL) was added NH₄Cl (470 mg, 8.8 mmol), HOBt (890 mg, 6.6 mmol) and EDCI (1.3 g, 6.6 mol). The reaction mixture was stirred at rt overnight. The reaction was quenched with aqueous NaHCO₃, diluted with brine, and the layers were separated. The organics were concentrated to afford crude 4-bromo-1-methyl-1H-pyrrole-2-carboxamide as an oil (1.6 g) that was carried on to the next step without further purification. LC/MS ESI (m/z): 203/205 (Br) (M+H)⁺.

### Step 3. 4-Bromo-1-methyl-1H-pyrrole-2-carbonitrile

To a solution of 4-bromo-1-methyl-1H-pyrrole-2-carboxamide (1.5 g from the previous step) in DCM (5 mL) and TEA (1.3 mL) was added TFAA (0.56 mL) at 0° C. and the mixture was stirred at 0° C. for 2 h. The reaction was quenched with NaHCO₃, diluted with brine, and the layers were separated, and the organics were concentrated. Purification by flash column chromatography (silica gel, 0˜30%, ethyl acetate in petroleum ether) provided 4-bromo-1-methyl-1H-pyrrole-2-carbonitrile as a yellow solid (330 mg, 80% yield over two steps). LC/MS ESI (m/z): 297/299 (Br) (M−H+TFA)

### Step 4. tert-Butyl (S)-4-(7-(5-cyano-1-methyl-1H-pyrrol-3-yl)-5-cyclopropyl-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (89 mg, 0.25 mmol, prepared following the procedure of compound 260, step 4) and 4-bromo-1-methyl-1H-pyrrole-2-carbonitrile (92 mg, 0.50 mmol) in DMF (1 mL) were added CuI (47 mg, 0.25 mmol), trans-N,N′-dimethylcyclohexane-1,2-diamine (35 mg, 0.25 mmol) and K₃PO₄ (530 mg, 2.5 mmol). The resulting mixture was stirred at 120° C. under N₂ atmosphere overnight. After being cooled to room temperature, the reaction was partitioned between EtOAc, and water and the organic layer was separated. The aqueous layer was extracted with EtOAc twice and the combined organic layers were washed with brine, dried over Na₂SO₄, filtered, and concentrated. The residue was purified by flash column chromatography (silica gel, 0˜40%, ethyl acetate in petroleum ether) to give crude product which was further purified by prep-HPLC to afford tert-butyl (S)-4-(7-(5-cyano-1-methyl-1H-pyrrol-3-yl)-5-cyclopropyl-7H-pyrrolo[2,3-d]pyrimidin-4-yl)-3-methylpiperazine-1-carboxylate (13 mg, 11% yield) as a white solid. LC/MS ESI (m/z): 462 (M+H)⁺. ¹H NMR (400 MHz, CDCl₃) δ 8.42 (s, 1H), 7.49 (s, 1H), 6.95 (s, 1H), 6.81 (s, 1H), 4.93-4.56 (m, 1H), 4.25-3.73 (m, 6H), 3.62-3.45 (m, 1H), 3.42-3.02 (m, 2H), 2.04-1.98 (m, 1H), 1.50 (s, 9H), 1.22 (d, J=6.3 Hz, 3H), 1.09-0.92 (m, 2H), 0.83-0.73 (m, 1H), 0.73-0.62 (m, 1H).

### Example 130. Synthesis of tert-butyl (S)-4-(7-(5-cyanopyridazin-3-yl)-5-(2-fluorophenyl)-7H-pyrrolo[2,3-d]pyrimidin-4-yl)-3-methylpiperazine-1-carboxylate (Compound 447)

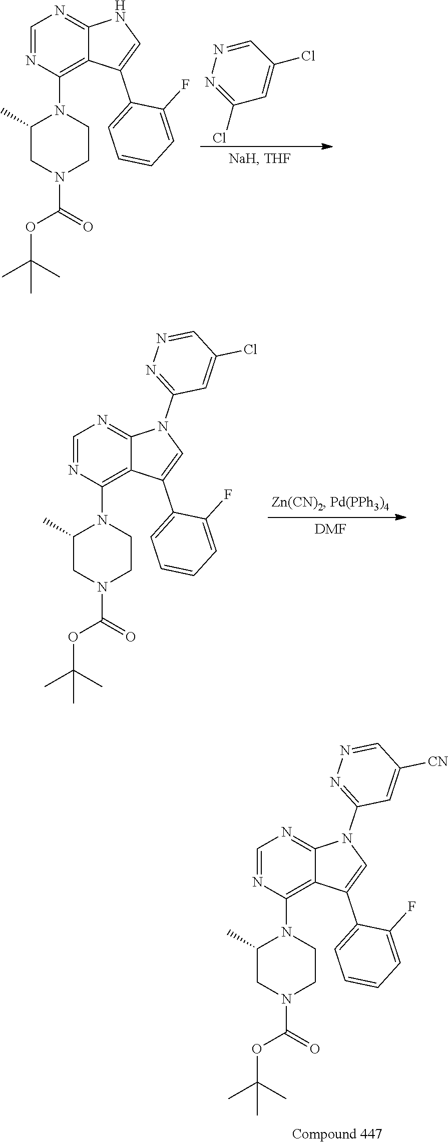

### Step 1. tert-Butyl (S)-4-(7-(5-chloropyridazin-3-yl)-5-(2-fluorophenyl)-7H-pyrrolo[2,3-d]pyrimidin-4-yl)-3-methylpiperazine-1-carboxylate

To a solution of tert-butyl (S)-4-(5-(2-fluorophenyl)-7H-pyrrolo[2,3-d]pyrimidin-4-yl)-3-methylpiperazine-1-carboxylate (82 mg, 0.20 mmol, prepared following the procedure of compound 268, step 4) in THE (1 mL) was added NaH (32 mg, 0.80 mmol, 60% wt.) at 0° C. and the reaction mixture was stirred for 20 min under N₂. Then 3,5-dichloropyridazine (60 mg, 0.40 mmol) was added and the mixture was stirred at room temperature under N₂ overnight. The mixture was quenched with NH₄Cl aqueous, diluted with EtOAc, washed with brine, dried over Na₂SO₄, filtered and concentrated. The residue was purified by flash column chromatography (silica gel, 0˜40% EtOAc in petroleum ether) to afford tert-butyl (S)-4-(7-(5-chloropyridazin-3-yl)-5-(2-fluorophenyl)-7H-pyrrolo[2,3-d]pyrimidin-4-yl)-3-methylpiperazine-1-carboxylate (36 mg, 34% yield) as a white solid. LC/MS ESI (m/z): 524/526 (Cl) (M+H)⁺. And tert-butyl (S)-4-(7-(6-chloropyridazin-4-yl)-5-(2-fluorophenyl)-7H-pyrrolo[2,3-d]pyrimidin-4-yl)-3-methylpiperazine-1-carboxylate (36 mg, 34%) as a white solid. LC/MS ESI (m/z): 524/526 (Cl) (M+H)⁺. The configuration was determined by NMR analysis.

### Step 2. tert-Butyl (S)-4-(7-(5-cyanopyridazin-3-yl)-5-(2-fluorophenyl)-7H-pyrrolo[2,3-d]pyrimidin-4-yl)-3-methylpiperazine-1-carboxylate

To a solution of tert-butyl (S)-4-(7-(5-chloropyridazin-3-yl)-5-(2-fluorophenyl)-7H-pyrrolo[2,3-d]pyrimidin-4-yl)-3-methylpiperazine-1-carboxylate (24 mg, 0.046 mmol) in DMF (1 mL) was added Zn(CN)₂ (32 mg, 0.28 mmol) and Pd(PPh₃)₄ (53 mg, 0.046 mmol). The mixture was stirred at 120° C. under N₂ overnight then diluted with EtOAc. The mixture was washed with brine, dried over Na₂SO₄, filtered, and concentrated. The residue was purified by flash column chromatography (silica gel, 0˜40% EtOAc in petroleum ether) to provide the product which was further purified by prep-HPLC to afford tert-butyl (S)-4-(7-(5-cyanopyridazin-3-yl)-5-(2-fluorophenyl)-7H-pyrrolo[2,3-d]pyrimidin-4-yl)-3-methylpiperazine-1-carboxylate (4.4 mg, 1900 yield) as a yellow solid. LC/MS ESI (m/z): 515 (M+H)⁺. ¹H NMR (400 MHz, CDCl₃) δ 9.78 (s, 1H), 9.22 (s, 1H), 8.54 (s, 1H), 8.47 (s, 1H), 7.47 (t, J=7.2 Hz, 1H), 7.40 (dd, J=13.3, 6.1 Hz, 1H), 7.26-7.16 (m, 2H), 4.43-4.11 (m, 1H), 3.91-3.63 (m, 1H), 3.57 (d, J=12.8 Hz, 1H), 3.48 (d, J=12.6 Hz, 1H), 3.08 (t, J=11.3 Hz, 1H), 2.95-2.51 (m, 2H), 1.43 (s, 9H), 1.03 (s, 3H). ¹⁹F NMR (377. Hz, CDCl₃) δ −114.54 (s).

The following compounds were prepared by analogous procedures to the synthesis of compound 447. The starting material of step 1 were prepared from the corresponding 2-fluorophenyl- or cyclopropyl-boronic acid.

| Cmpd No. | Chemical Name | LCMS and ¹H NMR |
|---|---|---|
| 448 | tert-butyl (S)-4-(7-(6- | LC/MS ESI (m/z): 515 (M + H)⁺. ¹H NMR (400 MHz, |
| | cyanopyridazin-4-yl)-5-(2- | CDCl₃) δ 9.99 (d, J = 2.5 Hz, 1H), 9.03 (d, J = 2.5 Hz, 1H), |
| | fluorophenyl)-7H-pyrrolo[2,3- | 8.56 (s, 1H), 7.58 (s, 1H), 7.50-7.36 (m, 2H), 7.34-7.27 |
| | d]pyrimidin-4-yl)-3- | (m, 1H), 7.26-7.20 (m, 1H), 4.42-4.14 (m, 1H), 3.90- |
| | methylpiperazine-1-carboxylate | 3.66 (m, 1H), 3.59 (d, J = 13.2 Hz, 1H), 3.55-3.35 (m, |
| | | 1H), 3.06 (t, J = 11.7 Hz, 1H), 2.97-2.50 (m, 2H), 1.43 (s, |
| | | 9H), 1.14-0.93 (m, 3H). ¹⁹F NMR (377 MHz, CDCl₃) δ - |
| | | 114.69 (s). |
| 439 | tert-butyl (S)-4-(7-(5- | LC/MS ESI (m/z): 515 (M + H)⁺. ¹H NMR (400 MHz, |
| | cyanopyridazin-3-yl)-5- | CDCl₃) δ 9.70 (d, J = 1.7 Hz, 1H), 9.15 (d, J = 1.7 Hz, 1H), |
| | cyclopropyl-7H-pyrrolo[2,3- | 8.48 (s, 1H), 8.00 (s, 1H), 4.89-4.59 (m, 1H), 4.29-3.76 |
| | d]pyrimidin-4-yl)-3- | (m, 3H), 3.55 (t, J = 11.9 Hz, 1H), 3.44-2.98 (m, 2H), |
| | methylpiperazine-1-carboxylate | 2.08-1.95 (m, 1H), 1.50 (s, 9H), 1.25 (d, J = 6.5 Hz, 3H), |
| | | 1.12-1.01 (m, 2H), 0.93-0.86 (m, 1H), 0.85-0.77 (m, 1H). |

### Example 131. Synthesis of tert-butyl (S)-4-(1-(4-cyanopyridin-2-yl)-3-(2-fluorophenyl)-1H-pyrrolo[3,2-c]pyridin-4-yl)-3-methylpiperazine-1-carboxylate (Compound 449)

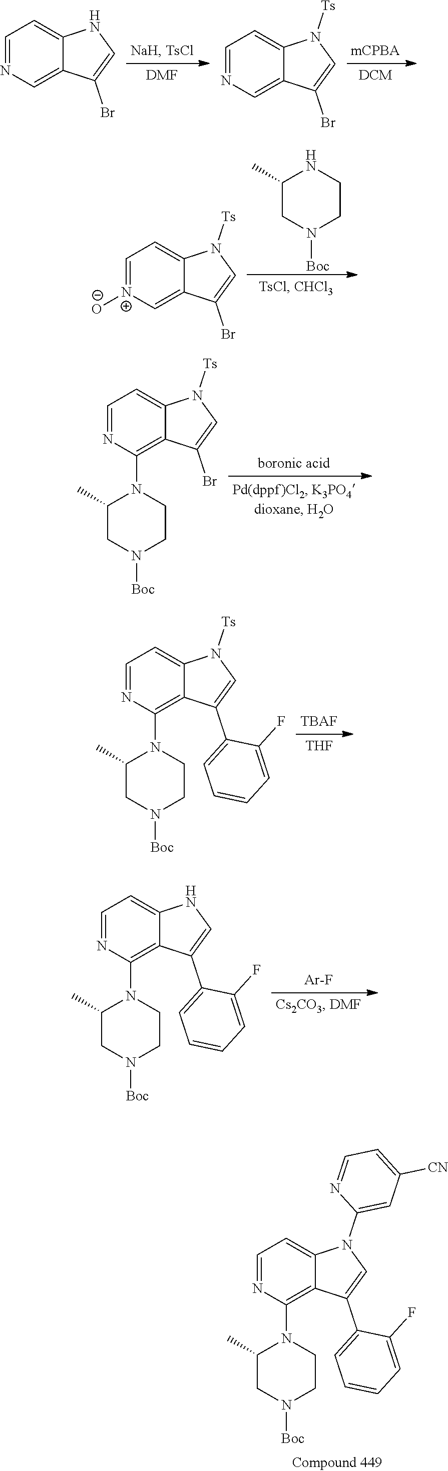

### Step 1. 3-Bromo-1-tosyl-1H-pyrrolo[3,2-c]pyridine

At 0° C., to a solution of NaH (1.2 g, 30 mmol) in DMF (50 mL) was added 3-bromo-1H-pyrrolo[3,2-c]pyridine (5.0 g, 25 mmol) and the mixture stirred at 0° C. for 15 min. TsCl (5.3 g, 28 mmol) was added and the resulting mixture was stirred at room temperature overnight under N₂. The mixture was diluted with water and filtered. The filter cake was washed with water and dried under vacuum to provide 3-bromo-1-tosyl-1H-pyrrolo[3,2-c]pyridine (7.5 g, 84%) as a white solid. LC/MS ESI (m/z): 351 (M+H)⁺.

### Step 2. 3-Bromo-1-tosyl-1H-pyrrolo[3,2-c]pyridine 5-oxide

At 0° C., to a solution of 3-bromo-1 3-bromo-1-tosyl-1H-pyrrolo[3,2-c]pyridine (7.0 g, 20 mmol) in DCM (70 mL) was added 3-chloroperoxybenzoic acid (5.2 g, 30 mmol) in portions. The resulting mixture was stirred at room temperature overnight. The reaction was quenched with NaHCO₃ (aq.), extracted with DCM twice, the combined organic layers were washed with brine, dried over Na₂SO₄, filtered, and concentrated. The residue was purified by flash column chromatography (silica gel, 0-20%, MeOH in DCM) to provide 3-bromo-1-tosyl-1H-pyrrolo[3,2-c]pyridine 5-oxide (4.0 g, 54%) as a light yellow solid. LC/MS ESI (m/z): 367, 369 (M+H)⁺

### Step 3. tert-Butyl (S)-4-(3-bromo-1-tosyl-1H-pyrrolo[3,2-c]pyridin-4-yl)-3-methylpiperazine-1-carboxylate

To a solution of 3-bromo-1-tosyl-1H-pyrrolo[3,2-c]pyridine 5-oxide (300 mg, 0.80 mmol) in CHCl₃ (5 mL) were added tert-butyl (S)-3-methylpiperazine-1-carboxylate (390 mg, 1.9 mmol) and the mixture stirred at 0° C. for 10 min. TsCl (180 mg, 0.96 mmol) was added and the resulting mixture was stirred at room temperature overnight under N₂. The reaction was quenched with NaHCO₃ (aq.), extracted with DCM twice, the combined organic layers were washed with brine, dried over Na₂SO₄, filtered, and concentrated. The residue was purified by flash column chromatography (silica gel, 0-40%, ethyl acetate in petroleum ether) to provide tert-butyl (S)-4-(3-bromo-1-tosyl-1H-pyrrolo[3,2-c]pyridin-4-yl)-3-methylpiperazine-1-carboxylate (47 mg, 10%) as a light yellow solid. LC/MS ESI (m/z): 549, 551 (M+H)⁺

### Step 4. tert-Butyl (S)-4-(3-(2-fluorophenyl)-1-tosyl-1H-pyrrolo[3,2-c]pyridin-4-yl)-3-methylpiperazine-1-carboxylate

To a solution of tert-butyl (S)-4-(3-bromo-1-tosyl-1H-pyrrolo[3,2-c]pyridin-4-yl)-3-methylpiperazine-1-carboxylate (62 mg, 0.10 mmol) in dioxane (5 mL) and H₂O (1 mL) were added (2-fluorophenyl)boronic acid (16 mg, 0.11 mmol), K₃PO₄ (43 mg, 0.20 mmol) and Pd(dppf)Cl₂ (8.0 mg, 0.010 mmol). The resulting mixture was heated to 90° C. overnight. After being cooled to room temperature, the solvent was removed and the residue was purified by flash column chromatography (silica gel, 0˜40%, ethyl acetate in petroleum ether) to afford tert-butyl (S)-4-(3-(2-fluorophenyl)-1-tosyl-1H-pyrrolo[3,2-c]pyridin-4-yl)-3-methylpiperazine-1-carboxylate (51 mg, 80%) as a white solid. LC/MS ESI (m/z): 565 (M+H)⁺.

### Step 5. tert-Butyl (S)-4-(3-(2-fluorophenyl)-1H-pyrrolo[3,2-c]pyridin-4-yl)-3-methylpiperazine-1-carboxylate

To a solution of tert-butyl (S)-4-(3-(2-fluorophenyl)-1-tosyl-1H-pyrrolo[3,2-c]pyridin-4-yl)-3-methylpiperazine-1-carboxylate (50 mg, 0.090 mmol) in THE (1 mL) was added TBAF (1.0 mL, 1.0M in THF). The resulting mixture was stirred at room temperature overnight under N₂. The reaction was quenched with water, extracted with EtOAc twice, the combined organic layers were washed with brine, dried over Na₂SO₄, filtered, and concentrated. The residue was purified by flash column chromatography (silica gel, 0-60%, ethyl acetate in petroleum ether) to provide tert-butyl (S)-4-(3-(2-fluorophenyl)-1H-pyrrolo[3,2-c]pyridin-4-yl)-3-methylpiperazine-1-carboxylate (35 mg, 96%) as a yellow solid. LC/MS ESI (m/z): 411 (M+H)⁺.

### Step 6. tert-Butyl (S)-4-(1-(4-cyanopyridin-2-yl)-3-(2-fluorophenyl)-1H-pyrrolo[3,2-c]pyridin-4-yl)-3-methylpiperazine-1-carboxylate

To a solution of tert-butyl (S)-4-(3-(2-fluorophenyl)-1H-pyrrolo[3,2-c]pyridin-4-yl)-3-methylpiperazine-1-carboxylate (35 mg, 0.080 mmol) in DMF (5 mL) were added 2-fluoroisonicotinonitrile (20 mg, 0.16 mmol), and Cs₂CO₃ (1.2 g, 8.3 mmol). The resulting mixture was heated to 50° C. overnight. After being cooled to room temperature, the solvent was removed and the residue was purified by flash column chromatography (silica gel, ethyl acetate in petroleum ether) to give the product which was further purified by prep-HPLC to afford tert-butyl (S)-4-(1-(4-cyanopyridin-2-yl)-3-(2-fluorophenyl)-1H-pyrrolo[3,2-c]pyridin-4-yl)-3-methylpiperazine-1-carboxylate (51 mg, 80%) as a white solid. LC/MS ESI (m/z): 513 (M+H)⁺. H NMR (400 MHz, CDCl₃) δ 8.77 (d, J=4.9 Hz, 1H), 8.23 (d, J=5.9 Hz, 1H), 7.88 (d, J=5.8 Hz, 1H), 7.71 (d, J=13.5 Hz, 2H), 7.53-7.44 (m, 2H), 7.42-7.35 (m, 1H), 7.24-7.17 (m, 2H), 3.47 (s, 1H), 3.12-2.86 (m, 6H), 1.42 (s, 9H), 0.91 (d, J=4.7 Hz, 3H).

The following compound was prepared by an analogous procedure to the synthesis of compound 449 from the corresponding boronic acid (step 4, cyclopropylboronic acid (1.5 eq), K₂CO₃ (20 eq), Pd-118 (0.05 eq), toluene, 90° C., overnight).

| Cmpd No. | Chemical Name | LCMS and ¹H NMR |
|---|---|---|
| 446 | tert-butyl (S)-4-(1-(4- | LC/MS ESI (m/z): 459 (M + H)⁺. ¹H NMR (400 MHz, |
| | cyanopyridin-2-yl)-3- | CDCl₃) δ 8.71 (d, J = 5.0 Hz, 1H), 8.17 (d, J = 5.8 Hz, 1H), |
| | cyclopropyl-1H-pyrrolo[3,2- | 7.84 (d, J = 4.7 Hz, 1H), 7.61 (s, 1H), 7.38 (d, J = 4.2 Hz, |
| | c]pyridin-4-yl)-3- | 1H), 7.18 (s, 1H), 3.99-3.84 (m, 1H), 3.76 (d, J = 11.9 Hz, |
| | methylpiperazine-1-carboxylate | 1H), 3.67-3.55 (m, 2H), 3.52-3.41 (m, 1H), 3.34-3.22 |
| | | (m, 1H), 3.18-3.06 (m, 1H), 2.62-2.42 (m, 1H), 1.49 (s, |
| | | 9H), 1.07-1.00 (m, 5H), 0.87-0.82 (m, 1H), 0.66-0.60 |
| | | (m, 1H). |

### Example 132. Synthesis of tert-butyl (S)-4-(1-(4-cyanopyridin-2-yl)-3-(2-fluorophenyl)-1H-pyrrolo[3,2-c]pyridin-4-yl)-3-methylpiperazine-1-carboxylate (Compound 450)

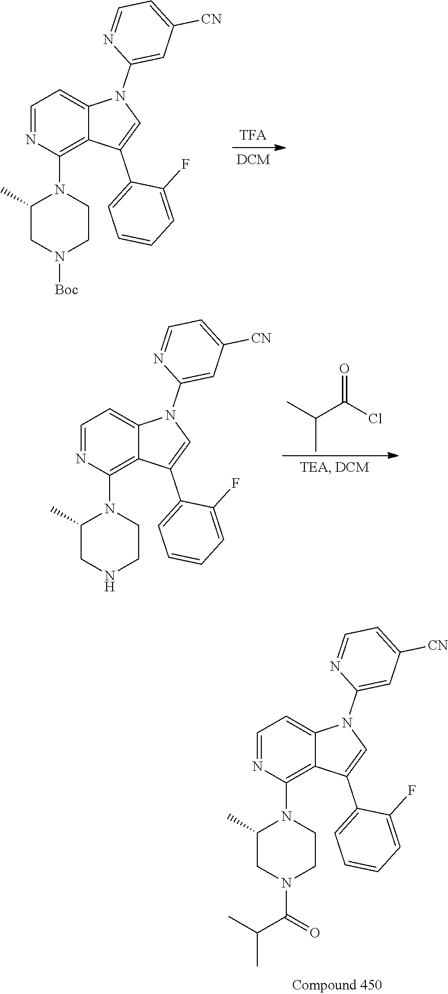

### Step 1. (S)-2-(3-(2-Fluorophenyl)-4-(2-methylpiperazin-1-yl)-1H-pyrrolo[3,2-c]pyridin-1-yl)isonicotinonitrile

At 0° C., to a solution of tert-butyl (S)-4-(1-(4-cyanopyridin-2-yl)-3-(2-fluorophenyl)-1H-pyrrolo[3,2-c]pyridin-4-yl)-3-methylpiperazine-1-carboxylate (130 mg, 0.24 mmol, prepared following the procedure of compound 449) in DCM (2 mL) was added TFA (1 mL). The resulting mixture was stirred at the same temperature for 1.5 h. The reaction was quenched with NaHCO₃ (aq.), the organic layer was separated, and the aqueous layer was extracted with DCM twice. The combined organic layers were washed with brine, dried over Na₂SO₄, filtered, and concentrated. The residue was used in the next step directly. (S)-2-(3-(2-fluorophenyl)-4-(2-methylpiperazin-1-yl)-1H-pyrrolo[3,2-c]pyridin-1-yl)isonicotinonitrile, LC/MS ESI (m/z): 413 (M+H)⁺.

### Step 2. (S)-2-(3-(2-Fluorophenyl)-4-(4-isobutyryl-2-methylpiperazin-1-yl)-1H-pyrrolo[3,2-c]pyridin-1-yl)isonicotinonitrile

At 0° C., to a solution of (S)-2-(3-(2-fluorophenyl)-4-(2-methylpiperazin-1-yl)-1H-pyrrolo[3,2-c]pyridin-1-yl)isonicotinonitrile (98 mg, 0.24 mmol) in DCM (3 mL) were added dropwise TEA (0.060 mL, 0.48 mmol) and isobutyryl chloride (0.040 mL, 0.36 mmol). The resulting mixture was stirred at room temperature for 1.5 h. The reaction was quenched with ice water and extracted with DCM twice. The combined organic layers were washed with brine, dried over Na₂SO₄, filtered, and concentrated. The residue was purified by flash column chromatography (silica gel, ethyl acetate in petroleum ether) to give the product which was further purified by prep-HPLC to afford (S)-2-(3-(2-fluorophenyl)-4-(4-isobutyryl-2-methylpiperazin-1-yl)-1H-pyrrolo[3,2-c]pyridin-1-yl)isonicotinonitrile (43 mg, 36%) as a white solid. LC/MS ESI (m/z): 483 (M+H)⁺. ¹H NMR (400 MHz, CDCl₃) δ 8.77 (d, J=4.9 Hz, 1H), 8.25-8.20 (m, 1H), 7.90 (s, 1H), 7.72 (d, J=11.9 Hz, 2H), 7.48 (dd, J=21.6, 5.9 Hz, 2H), 7.42-7.36 (m, 1H), 7.20 (dd, J=17.9, 8.3 Hz, 2H), 3.57-2.71 (m, 8H), 1.13-1.06 (m, 6H), 1.03 (d, J=6.1 Hz, 1.5H), 0.83 (d, J=6.3 Hz, 1.5H).

### Example 133. Synthesis of tert-butyl (S)-4-(5-(2-fluorophenyl)-7-(pyridazin-4-yl)-7H-pyrrolo[2,3-d]pyrimidin-4-yl)-3-methylpiperazine-1-carboxylate (Compound 451)

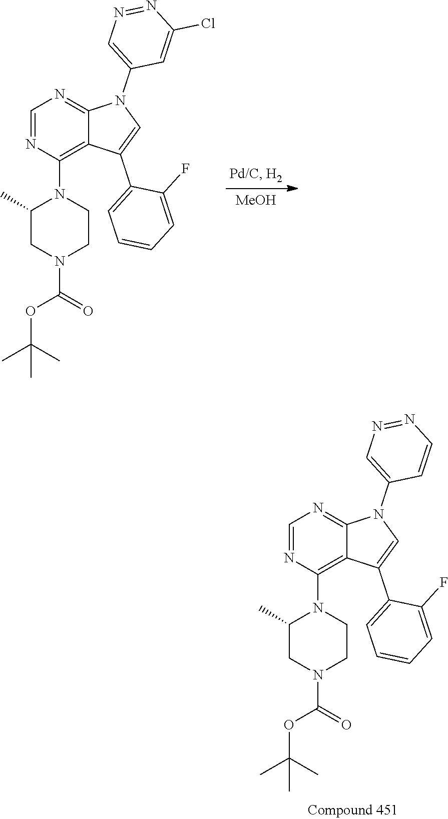 [SMILES: "MeOH" ; C[C@H]1CN(C(=O)OC(C)(C)C)CCN1c1ncnc2c1c(-c1ccccc1F)cn2-c1cnnc(Cl)c1 ; "Pd/C, H2" ; C[C@H]1CN(C(=O)OC(C)(C)C)CCN1c1ncnc2c1c(-c1ccccc1F)cn2-c1ccnnc1]

### Step 1. tert-Butyl (S)-4-(5-(2-fluorophenyl)-7-(pyridazin-4-yl)-7H-pyrrolo[2,3-d]pyrimidin-4-yl)-3-methylpiperazine-1-carboxylate

To a solution of tert-butyl (S)-4-(7-(6-chloropyridazin-4-yl)-5-(2-fluorophenyl)-7H-pyrrolo[2,3-d]pyrimidin-4-yl)-3-methylpiperazine-1-carboxylate (10 mg, 0.019 mmol, prepared following the procedure of compound 447, step 2) in MeOH (1 mL) was added Pd/C (10 mg, with ca. 55% water) under N₂. The mixture was stirred at room temperature under a H₂ atmosphere for 3 h and the solid was removed by filtration. The filtrate was concentrated to afford the product which was further purified by prep-HPLC to afford tert-butyl (S)-4-(5-(2-fluorophenyl)-7-(pyridazin-4-yl)-7H-pyrrolo[2,3-d]pyrimidin-4-yl)-3-methylpiperazine-1-carboxylate (1.9 mg, 20% yield) as a white solid. LC/MS ESI (m/z): 490 (M+H)⁺. ¹H NMR (400 MHz, CDCl₃) δ 9.85 (d, J=2.3 Hz, 1H), 9.29 (d, J=5.8 Hz, 1H), 8.55 (s, 1H), 8.37 (dd, J=5.8, 2.7 Hz, 1H), 7.57 (s, 1H), 7.43 (dt, J=19.3, 6.6 Hz, 2H), 7.30-7.28 (m, 1H), 7.24-7.21 (m, 1H), 4.38-4.13 (m, 1H), 3.89-3.66 (m, 1H), 3.60-3.39 (m, 2H), 3.08 (t, J=11.9 Hz, 1H), 2.91-2.58 (m, 2H), 1.43 (s, 9H), 1.08-0.96 (m, 3H).

### Example 134. Synthesis of tert-butyl (2R,5S)-4-(7-(4-chloropyridin-2-yl)-5-(2-fluorophenyl)-7H-pyrrolo[2,3-d]pyrimidin-4-yl)-2,5-dimethylpiperazine-1-carboxylate (Compound 454)

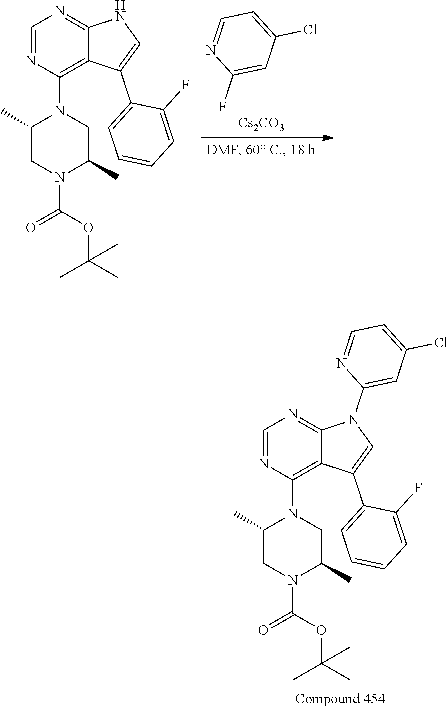

### Step 1. tert-Butyl(2R,5S)-4-(5-(2-fluorophenyl)-7-(4-fluoropyridin-2-yl)-7H-pyrrolo[2,3-d]pyrimidin-4-yl)-2,5-dimethylpiperazine-1-carboxylate

To a solution of tert-butyl(2R,5S)-4-[5-(2-fluorophenyl)-7H-pyrrolo[2,3-d]pyrimidin-4-yl]-2,5-dimethylpiperazine-1-carboxylate (340 mg, 0.80 mmol, prepared following the procedure outlined in compound 452) and 4-chloro-2-fluoropyridine (210 mg, 1.6 mmol) in DMF (5 mL) was added Cs₂CO₃ (1000 mg, 3.2 mmol). The resulting mixture was stirred at 60° C. for 18 h. After being cooled to room temperature, the reaction was partitioned between EtOAc and water, the organic layer was separated, and the aqueous layer was extracted with EtOAc twice. The combined organic phases were washed with brine, dried over anhydrous Na₂SO₄, filtered, and concentrated. The residue was purified by column chromatography on silica gel (0˜50% ethyl acetate in petroleum ether) to give tert-butyl(2R,5S)-4-[7-(4-chloropyridin-2-yl)-5-(2-fluorophenyl)-7H-pyrrolo[2,3-d]pyrimidin-4-yl]-2,5-dimethylpiperazine-1-carboxylate (370 mg, 86%) as a white solid. LC/MS (ESI) (m/z): 537 (M+H)^{+ 1}H NMR (400 MHz, CDCl₃) δ 9.06 (d, J=1.5 Hz, 1H), 8.57 (s, 1H), 8.37 (d, J=5.3 Hz, 1H), 8.27 (s, 1H), 7.49 (td, J=7.5, 1.5 Hz, 1H), 7.37 (dd, J=13.1, 5.8 Hz, 1H), 7.27-7.14 (m, 3H), 4.34-4.07 (m, 2H), 3.44-3.18 (m, 3H), 2.89 (dd, J=28.9, 18.9 Hz, 1H), 1.43 (s, 9H), 1.07 (d, J=6.8 Hz, 3H), 0.96 (d, J=6.6 Hz, 3H).

### Example 135. Synthesis of tert-butyl (S)-4-(7-(5-cyano-1-methyl-1H-pyrrol-2-yl)-5-cyclopropyl-7H-pyrrolo[2,3-d]pyrimidin-4-yl)-3-methylpiperazine-1-carboxylate (Compound 456)

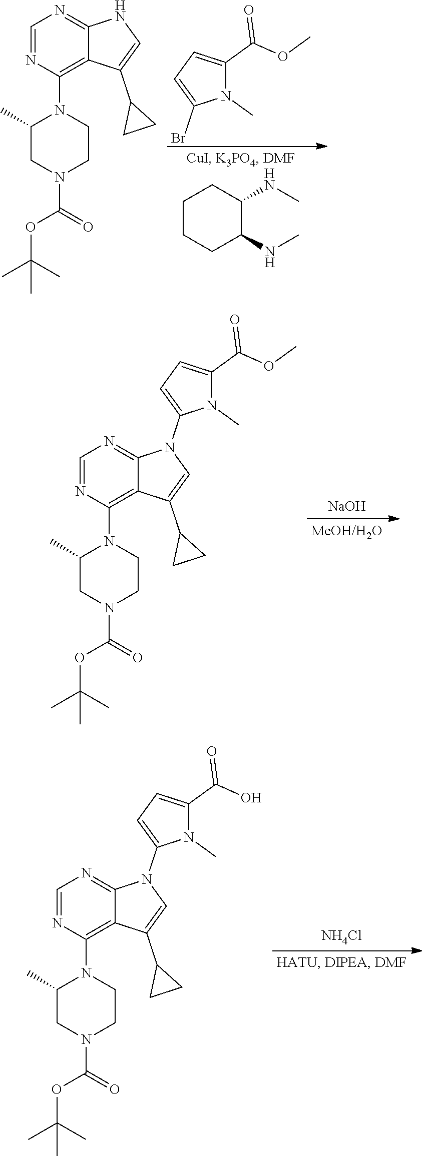

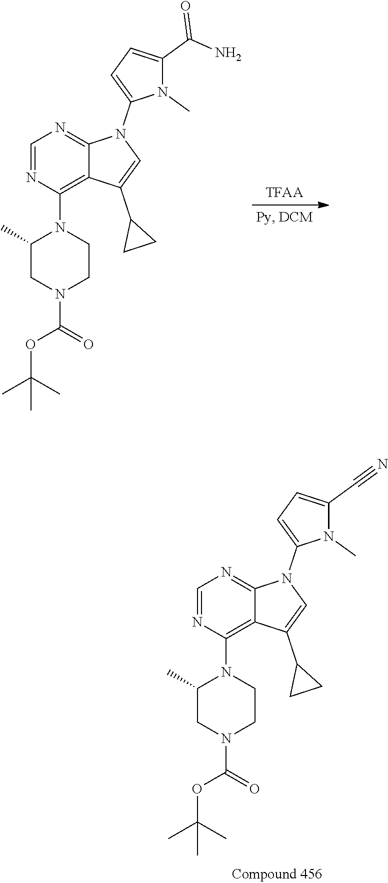 [SMILES: C[C@H]1CN(C(=O)OC(C)(C)C)CCN1c1ncnc2c1c(C1CC1)cn2-c1ccc(C(N)=O)n1C ; "TFAA" ; "Py, DCM" ; C[C@H]1CN(C(=O)OC(C)(C)C)CCN1c1ncnc2c1c(C1CC1)cn2-c1ccc(C#N)n1C]

### Step 1. tert-Butyl (S)-4-(5-cyclopropyl-7-(5-(methoxycarbonyl)-1-methyl-1H-pyrrol-2-yl)-7H-pyrrolo[2,3-d]pyrimidin-4-yl)-3-methylpiperazine-1-carboxylate

To a solution of tert-butyl (S)-4-(5-cyclopropyl-7H-pyrrolo[2,3-d]pyrimidin-4-yl)-3-methylpiperazine-1-carboxylate (200 mg, 0.50 mmol, prepared following a similar procedure outlined in compound 259) in dry DMF was added methyl 5-bromo-1-methyl-1H-pyrrole-2-carboxylate (220 mg, 1.0 mmol), K₃PO₄ (320 mg, 1.5 mmol) and CuI (48 mg, 0.25 mmol). trans-1,2-Cyclohexanediamine (72 mg, 0.50 mmol) was added, and the mixture was stirred at 120° C. under N₂ overnight. The mixture was cooled to rt, diluted with EtOAc, washed with H₂O, brine, dried over Na₂SO₄, filtered, and concentrated. The residue was purified by flash column chromatography (silica gel, 0˜40% EtOAc in petroleum ether) to provide tert-butyl (S)-4-(5-cyclopropyl-7-(5-(methoxycarbonyl)-1-methyl-1H-pyrrol-2-yl)-7H-pyrrolo[2,3-d]pyrimidin-4-yl)-3-methylpiperazine-1-carboxylate (220 mg, 88%) as a yellow foam. LC/MS ESI (m/z): 495 (M+H)⁺.

### Step 2. (S)-5-(4-(4-(tert-Butoxycarbonyl)-2-methylpiperazin-1-yl)-5-cyclopropyl-7H-pyrrolo[2,3-d]pyrimidin-7-yl)-1-methyl-1H-pyrrole-2-carboxylic acid

To the solution of tert-butyl (S)-4-(5-cyclopropyl-7-(5-(methoxycarbonyl)-1-methyl-1H-pyrrol-2-yl)-7H-pyrrolo[2,3-d]pyrimidin-4-yl)-3-methylpiperazine-1-carboxylate (220 mg, 0.45 mmol) in MeOH (10 mL) and H₂O (10 mL) was added NaOH (89 mg, 2.2 mmol). The resulting reaction mixture was stirred at 90° C. overnight. The reaction was quenched with 1 N HCl to PH 4 and extracted with EtOAc twice. The combined organic layers were dried over Na₂SO₄, filtered and concentrated to afford (S)-5-(4-(4-(tert-butoxycarbonyl)-2-methylpiperazin-1-yl)-5-cyclopropyl-7H-pyrrolo[2,3-d]pyrimidin-7-yl)-1-methyl-1H-pyrrole-2-carboxylic acid (180 mg, 84%) as a light yellow solid. LC/MS ESI (m/z): 481 (M+H)⁺.

### Step 3. tert-Butyl (S)-4-(7-(5-carbamoyl-1-methyl-1H-pyrrol-2-yl)-5-cyclopropyl-7H-pyrrolo[2,3-d]pyrimidin-4-yl)-3-methylpiperazine-1-carboxylate

To a solution of (S)-5-(4-(4-(tert-butoxycarbonyl)-2-methylpiperazin-1-yl)-5-cyclopropyl-7H-pyrrolo[2,3-d]pyrimidin-7-yl)-1-methyl-1H-pyrrole-2-carboxylic acid (180 mg, 0.38 mmol) in DMF (10 mL) were added HATU (210 mg, 0.56 mmol), DIPEA (190 mg, 1.5 mmol) and NH₄Cl (40 mg, 0.75 mmol) respectively. The resulting reaction mixture was stirred at r.t. under N₂ overnight. The reaction mixture was partitioned between EtOAc and water and the organic layer was separated. The aqueous layer was extracted with EtOAc twice and the combined organic layers were washed with brine, dried over Na₂SO₄, filtered, and concentrated under reduced pressure to afford tert-butyl (S)-4-(7-(5-carbamoyl-1-methyl-1H-pyrrol-2-yl)-5-cyclopropyl-7H-pyrrolo[2,3-d]pyrimidin-4-yl)-3-methylpiperazine-1-carboxylate (120 mg, 67%) as yellow solid. LC/MS ESI (m/z): 480 (M+H)⁺.

### Step 4. tert-Butyl (S)-4-(7-(5-cyano-1-methyl-1H-pyrrol-2-yl)-5-cyclopropyl-7H-pyrrolo[2,3-d]pyrimidin-4-yl)-3-methylpiperazine-1-carboxylate

A mixture of tert-butyl (S)-4-(7-(5-carbamoyl-1-methyl-1H-pyrrol-2-yl)-5-cyclopropyl-7H-pyrrolo[2,3-d]pyrimidin-4-yl)-3-methylpiperazine-1-carboxylate (120 mg, 0.25 mmol) and pyridine (400 mg, 5.0 mmol) in DCM (10 mL) was stirred at 0° C. Then TFAA (260 mg, 1.3 mmol) was dropwise at 0° C., the mixture stirred at 0° C. for 0.5 h, and extracted with H₂O and DCM. The organic layers were washed with brine, dried over Na₂SO₄, filtered, and concentrated under reduced pressure. Purification by flash column chromatography (silica gel, 0˜30% EtOAc in petroleum ether) followed by prep-HPLC provided tert-butyl (S)-4-(7-(5-cyano-1-methyl-1H-pyrrol-2-yl)-5-cyclopropyl-7H-pyrrolo[2,3-d]pyrimidin-4-yl)-3-methylpiperazine-1-carboxylate (75 mg, 65%) as a light yellow solid. LC/MS ESI (m/z): 462 (M+H)⁺. ¹H NMR (400 MHz, CDCl₃) δ 8.32 (s, 1H), 6.79 (d, J=4.1 Hz, 1H), 6.60 (s, 1H), 6.12 (d, J=4.1 Hz, 1H), 4.83-4.68 (m, 1H), 4.11-3.77 (m, 3H), 3.54-3.43 (m, 4H), 3.29-2.97 (m, 2H), 1.96-1.89 (m, 1H), 1.43 (s, 9H), 1.20 (d, J=6.2 Hz, 3H), 0.98-0.91 (m, 2H), 0.72-0.62 (m, 2H).

The following compounds were prepared by procedures analogous to the synthesis of compound 456 from the corresponding aryl bromide.

| Cmpd No. | Chemical Name | LCMS and ¹H NMR |
|---|---|---|
| 453 | tert-butyl (S)-4-(5-cyclopropyl- | LC/MS ESI (m/z): 453 (M + H)⁺ ¹H NMR(400 MHz, |
| | 7-(4-fluoropyridin-2-yl)-7H- | CDCl3) δ 8.71-8.59 (m, 1H), 8.48-8.39 (m, 1H), 8.37- |
| | pyrrolo[2,3-d]pyrimidin-4-yl)-3- | 8.27 (m, 1H), 7.82-7.66 (m, 1H), 6.88-6.78 (m, 1H), |
| | methylpiperazine-1-carboxylate | 4.66-4.59 (m, 1H), 4.09-3.75 (m, 3H), 3.53-3.44 (m, |
| | | 1H), 3.32-3.22 (m, 1H), 3.15-3.01 (m, 1H), 2.01-1.93 |
| | | (m, 1H), 1.44-1.42 (m, 9H), 1.18-1.14 (m, 3H), 0.97- |
| | | 0.93 (m, 2H), 0.83-0.78 (m, 1H), 0.72-0.66 (m, 1H). |

### Example 136. Synthesis of tert-butyl(S)-4-(7-(4-chloropyridin-2-yl)-5-cyclopropyl-7H-pyrrolo[2,3-d]pyrimidin-4-yl)-3-methylpiperazine-1-carboxylate (Compound 457)

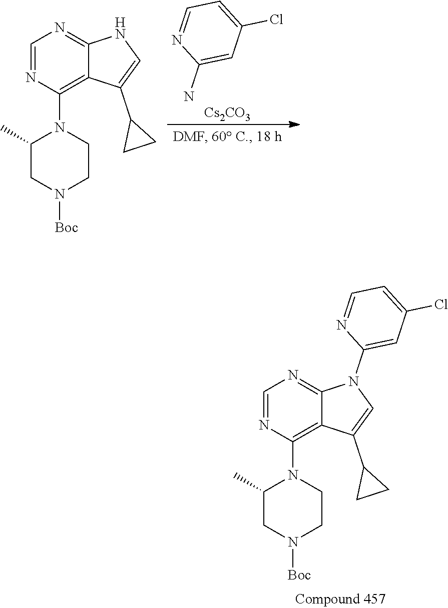

### Step 1. tert-Butyl(S)-4-(7-(4-chloropyridin-2-yl)-5-cyclopropyl-7H-pyrrolo[2,3-d]pyrimidin-4-yl)-3-methylpiperazine-1-carboxylate

To a solution of tert-butyl(3S)-4-{5-cyclopropyl-7H-pyrrolo[2,3-d]pyrimidin-4-yl}-3-methylpiperazine-1-carboxylate (110 mg, 0.30 mmol, prepared following the procedure outlined in compound 259) and 4-chloro-2-fluoropyridine (99 mg, 0.75 mmol) in DMF (3 mL) was added Cs₂CO₃ (490 mg, 1.5 mmol). The resulting mixture was stirred at 60° C. for 18 h. After being cooled to room temperature, the reaction was partitioned between EtOAc and water, the organic layer was separated, and the aqueous layer was extracted with EtOAc twice. The combined organic phases were washed with brine, dried over anhydrous Na₂SO₄, filtered, and concentrated. The residue was purified by column chromatography on silica gel (0˜50% ethyl acetate in petroleum ether) to give tert-butyl (S)-4-(7-(4-chloropyridin-2-yl)-5-cyclopropyl-7H-pyrrolo[2,3-d]pyrimidin-4-yl)-3-methylpiperazine-1-carboxylate (60 mg, 42%) as a white solid. LC/MS (ESI) (m/z): 469 (M+H)^{+ 1}H NMR (400 MHz, CDCl₃) δ 8.96 (d, J=1.4 Hz, 1H), 8.49 (s, 1H), 8.32 (d, J=5.3 Hz, 1H), 7.74 (s, 1H), 7.14 (dd, J=5.3, 1.7 Hz, 1H), 4.68 (s, 1H), 4.18-3.75 (m, 3H), 3.53 (t, J=11.3 Hz, 1H), 3.41-3.02 (m, 2H), 2.03 (d, J=5.9 Hz, 1H), 1.49 (s, 9H), 1.21 (d, J=6.4 Hz, 3H), 1.01 (dd, J=8.0, 1.6 Hz, 2H), 0.91-0.73 (m, 2H).

### Example 137. Synthesis of tert-butyl (2R,5S)-4-(7-(4-cyanopyrimidin-2-yl)-5-(2-fluorophenyl)-7H-pyrrolo[2,3-d]pyrimidin-4-yl)-2,5-dimethylpiperazine-1-carboxylate (Compound 460)

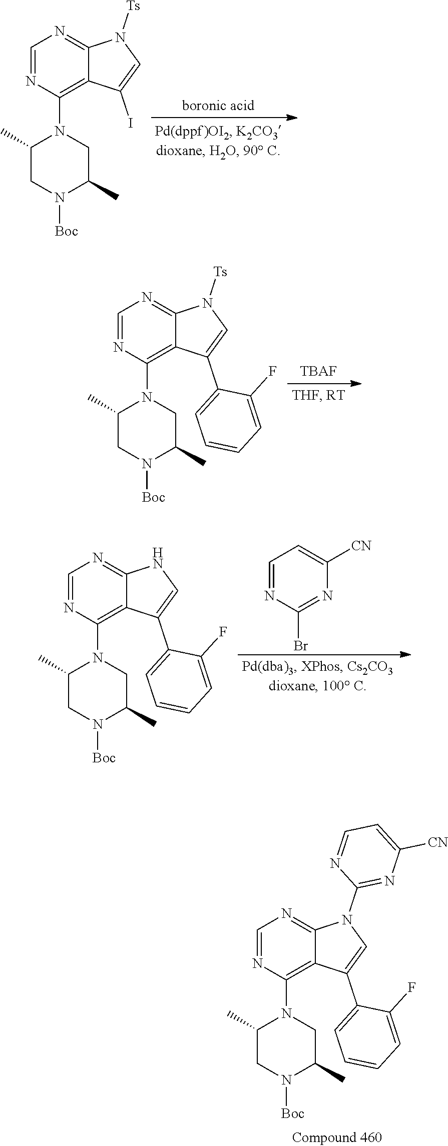

### Step 1. tert-Butyl (2R,5S)-4-(5-(2-fluorophenyl)-7-tosyl-7H-pyrrolo[2,3-d]pyrimidin-4-yl)-2,5-dimethylpiperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (300 mg, 0.49 mmol, prepared following the procedure outlined in compound 252) in dioxane (6 mL) and H₂O (1 mL) were added (2-fluorophenyl)boronic acid (140 mg, 0.98 mmol), K₂CO₃ (200 g, 1.5 mmol) and Pd(dppf)Cl₂ (37 mg, 0.050 mmol). The resulting mixture was heated to 90° C. overnight under N₂. After being cooled to room temperature, the solvent was removed and the residue was purified by flash column chromatography (silica gel, 0˜40%, ethyl acetate in petroleum ether) to afford tert-butyl (2R,5S)-4-(5-(2-fluorophenyl)-7-tosyl-7H-pyrrolo[2,3-d]pyrimidin-4-yl)-2,5-dimethylpiperazine-1-carboxylate (200 mg, 70%) as a solid. LC/MS ESI (m/z): 580 (M+H)⁺.

### Step 2. tert-Butyl (2R,5S)-4-(5-(2-fluorophenyl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7-tosyl-7H-pyrrolo[2,3-d]pyrimidin-4-yl)-2,5-dimethylpiperazine-1-carboxylate (100 mg, 0.17 mmol) in THE (5 mL) was added TBAF (3.0 mL, 1.0 M in THF) and the resulting mixture was stirred at room temperature overnight. The reaction was quenched with water and extracted with EtOAc twice. The combined organic layers were washed with brine, dried over Na₂SO₄, filtered, and concentrated to afford tert-butyl (2R,5S)-4-(5-(2-fluorophenyl)-7-tosyl-7H-pyrrolo[2,3-d]pyrimidin-4-yl)-2,5-dimethylpiperazine-1-carboxylate (70 mg, 95%) as a solid. LC/MS ESI (m/z): 426 (M+H)⁺.

### Step 3. tert-Butyl (2R,5S)-4-(7-(4-cyanopyrimidin-2-yl)-5-(2-fluorophenyl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7-tosyl-7H-pyrrolo[2,3-d]pyrimidin-4-yl)-2,5-dimethylpiperazine-1-carboxylate (50 mg, 0.13 mmol) in dioxane (5 mL) were added 2-bromopyrimidine-4-carbonitrile (35 mg, 0.19 mmol), Pd(dba)₃ (64 mg, 0.70 mmol) and X-Phos (75 mg, 0.13 mmol) and Cs₂CO₃ (85 mg, 0.26 mmol). The resulting mixture was heated to 100° C. overnight under N₂. The mixture was cooled, concentrated, and the residue was purified by flash column chromatography (silica gel, 0-10%, MeOH in DCM) to afford tert-butyl (2R,5S)-4-(7-(4-cyanopyrimidin-2-yl)-5-(2-fluorophenyl)-7H-pyrrolo[2,3-d]pyrimidin-4-yl)-2,5-dimethylpiperazine-1-carboxylate (20 mg, 30%). 20 mg of which was further purified by prep-HPLC to afford 10 mg of white solid. LC/MS ESI (m/z): 529 (M+H)⁺. ¹H NMR (400 MHz, CD₃OD) δ 9.13 (d, J=4.9 Hz, 1H), 8.53 (s, 1H), 8.20 (s, 1H), 7.84 (d, J=4.9 Hz, 1H), 7.60-7.55 (m, 1H), 7.53-7.47 (m, 1H), 7.35 (t, J=7.6 Hz, 1H), 7.29 (t, J=9.1 Hz, 1H), 4.24-4.10 (m, 2H), 3.42-3.35 (m, 2H), 3.24-3.19 (m, 1H), 2.82 (s, 1H), 1.43 (s, 9H), 1.06 (d, J=6.8 Hz, 3H), 0.95 (d, J=6.7 Hz, 3H).

The following compounds were prepared by analogous procedures to the synthesis of compound 460 from the corresponding aryl halide.

| Cmpd No. | Chemical Name | LCMS and ¹H NMR |
|---|---|---|
| 459 | tert-butyl 4-(7-(4- | ¹H NMR (400 MHz, CD₃OD) δ 9.13 (d, J = 4.8 Hz, 1H), |
| | cyanopyrimidin-2-yl)-5-(2- | 8.55 (s, 1H), 8.21 (s, 1H), 7.84 (d, J = 4.9 Hz, 1H), 7.60- |
| | fluorophenyl)-7H-pyrrolo[2,3- | 7.54 (m, 1H), 7.52-7.45 (m, 1H), 7.37-7.27 (m, 2H), |
| | d]pyrimidin-4-yl)piperazine-1- | 3.28-3.25 (m, 4H), 3.16 (s, 4H), 1.42 (s, 9H). |
| | carboxylate | |

### Example 138. Synthesis of tert-butyl (2R,5S)-4-(7-(3,5-difluorophenyl)-5-(3-methylpyrazin-2-yl)-7H-pyrrolo[2,3-d]pyrimidin-4-yl)-2,5-dimethylpiperazine-1-carboxylate (Compound 462)

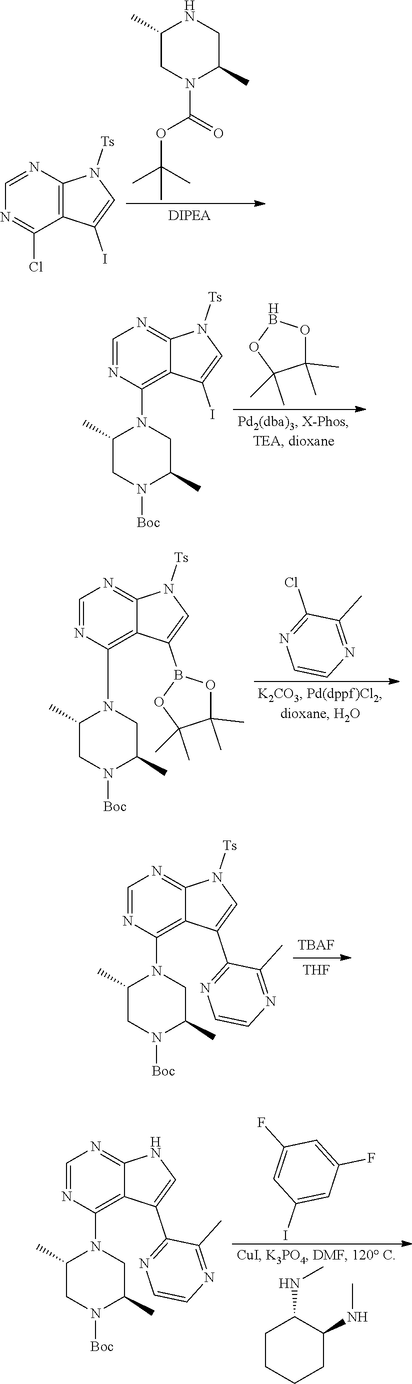

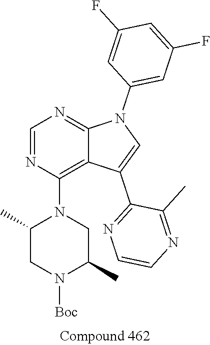 [SMILES: Cc1nccnc1-c1cn(-c2cc(F)cc(F)c2)c2ncnc(N3C[C@@H](C)N(C(=O)OC(C)(C)C)C[C@@H]3C)c12]

### Step 1. tert-Butyl (2R,5S)-4-(5-iodo-7-tosyl-7H-pyrrolo[2,3-d]pyrimidin-4-yl)-2,5-dimethylpiperazine-1-carboxylate

To a solution of 4-chloro-5-iodo-7-tosyl-7H-pyrrolo[2,3-d]pyrimidine (12 g, 28 mmol) in DIPEA (15 mL) was added tert-butyl (2R,5S)-2,5-dimethylpiperazine-1-carboxylate (9.0 g, 42 mmol). The resulting mixture was heated to 150° C. for 3 h under N₂. After being cooled to room temperature, the solvent was removed and the residue was purified by flash column chromatography (silica gel, 0-30%, ethyl acetate in petroleum ether) to afford tert-butyl (2R,5S)-4-(5-iodo-7-tosyl-7H-pyrrolo[2,3-d]pyrimidin-4-yl)-2,5-dimethyl piperazine-1-carboxylate (16 g, 94%) as a light yellow solid. LC/MS ESI (m/z): 612 (M+H)⁺.

### Step 2. tert-Butyl (2R,5S)-2,5-dimethyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (2.0 g, 3.2 mmol) in dioxane (20 mL) were added 4,4,5,5-tetramethyl-1,3,2-dioxaborolane (1.7 g, 13 mmol), TEA (2.2 mL, 16 mmol), X-Phos (0.31 g, 0.65 mmol) and Pd₂(dba)₃ (0.30 g, 0.32 mmol). The resulting mixture was stirred at 95° C. overnight then cooled to room temperature. The reaction was quenched with water and extracted with DCM twice. The combined organic layers were washed with brine, dried over Na₂SO₄, filtered, and concentrated to afford tert-butyl (2R,5S)-2,5-dimethyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate which was used in directly in the next step without further purification. LC/MS ESI (m/z): 612 (M+H)⁺.

### Step 3. tert-Butyl (2R,5S)-2,5-dimethyl-4-(5-(3-methylpyrazin-2-yl)-7-tosyl-7H-pyrrolo[2,3-d]pyrimidin-4-yl)piperazine-1-carboxylate

To a solution of tert-butyl (2R,5S)-2,5-dimethyl-4-(5-(4,4,5,5-tetramethyl-1,3,2-dioxaborolan-2-yl)-7-tosyl-7H-pyrrolo[2,3-d]pyrimidin-4-yl)piperazine-1-carboxylate (2.0 g, 3.2 mmol) in dioxane (20 mL) and H₂O (4 mL) were added 2-chloro-3-methylpyrazine (0.92 g, 7.2 mmol), K₂CO₃ (2.5 g, 18 mmol) and Pd(dppf)Cl₂ (0.26 g, 0.36 mmol). The resulting mixture was heated to 90° C. overnight. The mixture was cooled to room temperature and the solvent was removed. The residue was purified by flash column chromatography (silica gel, 0˜50%, ethyl acetate in petroleum ether) to afford tert-butyl (2R,5S)-2,5-dimethyl-4-(5-(3-methylpyrazin-2-yl)-7-tosyl-7H-pyrrolo[2,3-d]pyrimidin-4-yl)piperazine-1-carboxylate (1.3 g, 62%) as a yellow solid. LC/MS ESI (m/z): 578 (M+H)⁺.

### Step 4. tert-Butyl (2R,5S)-2,5-dimethyl-4-(5-(3-methylpyrazin-2-yl)-7H-pyrrolo[2,3-d]pyrimidin-4-yl)piperazine-1-carboxylate

To a solution of tert-butyl (2R,5S)-2,5-dimethyl-4-(5-(3-methylpyrazin-2-yl)-7-tosyl-7H-pyrrolo[2,3-d]pyrimidin-4-yl)piperazine-1-carboxylate (1.3 g, 2.2 mmol) in THE (10 mL) was added TBAF (10 mL, 1.0M in THF). The resulting mixture was stirred at room temperature for 2 hr. The reaction was quenched with water and extracted with EtOAc twice. The combined organic layers were washed with NH₄Cl (aq.), dried over Na₂SO₄, filtered and, concentrated to afford crude tert-butyl (2R,5S)-2,5-dimethyl-4-(5-(3-methylpyrazin-2-yl)-7H-pyrrolo[2,3-d]pyrimidin-4-yl)piperazine-1-carboxylate (950 mg, 99%) as a brown solid. LC/MS ESI (m/z): 424 (M+H)⁺.

### Step 5. tert-Butyl (R)-4-(7-(3,5-difluorophenyl)-5-(pyridin-2-yl)-7H-pyrrolo[2,3-d]pyrimidin-4-yl)-2-methylpiperazine-1-carboxylate

To a solution of tert-butyl (2R,5S)-2,5-dimethyl-4-(5-(3-methylpyrazin-2-yl)-7H-pyrrolo[2,3-d]pyrimidin-4-yl)piperazine-1-carboxylate (480 mg, 1.1 mmol) in DMF (5 mL) were added 1,3-difluoro-5-iodobenzene (310 mg, 1.3 mmol), trans-N,N′-dimethylcyclohexane-1,2-diamine (71 mg, 0.50 mmol), CuI (210 mg, 1.1 mmol) and K₃PO₄ (700 mg, 3.3 mmol). The resulting mixture was heated to 120° C. overnight then cooled to room temperature. The reaction was quenched with water and extracted with EtOAc twice. The combined organic layers were washed with brine, dried over Na₂SO₄, filtered, and concentrated. The residue was purified by flash column chromatography (silica gel, 0-30%, ethyl acetate in petroleum ether) to give the product which was further purified by prep-HPLC to afford tert-butyl (2R,5S)-4-(7-(3,5-difluorophenyl)-5-(3-methylpyrazin-2-yl)-7H-pyrrolo[2,3-d]pyrimidin-4-yl)-2,5-dimethylpiperazine-1-carboxylate (780 mg, 84%) as a white solid. LC/MS ESI (m/z): 536 (M+H)⁺. H NMR (400 MHz, CDCl₃) δ 8.57-8.51 (m, 3H), 7.49-7.43 (m, 3H), 6.84 (t, J=8.7 Hz, 1H), 4.09 (d, J=58.8 Hz, 2H), 3.31 (dd, J=24.7, 13.9 Hz, 2H), 3.09 (s, 1H), 2.95-2.66 (m, 1H), 2.53 (s, 3H), 1.43 (s, 9H), 1.02-0.97 (m, 6H).

The following compound was prepared by an analogous procedure to the synthesis of compound 462 from the corresponding amine and aryl halide.

| Cmpd | | |
|---|---|---|
| No. | Chemical Name | LCMS and ¹H NMR |
| 319 | tert-butyl (S)-4-(7-(4- | LC/MS ESI (m/z): 512.2 (M + H)⁺. ¹H NMR (400 MHz, |
| | cyanopyridin-2-yl)-5-(3- | CD₃OD) δ 9.32-9.30 (m, 1H), 8.71 (dd, J = 5.0, 0.7 Hz, |
| | methylpyrazin-2-yl)-7H- | 1H), 8.61-8.58 (m, 3H), 8.43 (s, 1H), 7.63 (dd, J = 5.0, 1.3 |
| | pyrrolo[2,3-d]pyrimidin-4-yl)-3- | Hz, 1H), 4.03 (br, 1H), 3.72 (d, J = 12.9 Hz, 1H), 3.49 (dd, |
| | methylpiperazine-1-carboxylate | J = 23.0, 13.2 Hz, 2H), 3.09-2.99 (m, 1H), 2.81-2.56 (m, |
| | | 2H), 2.54 (s, 3H), 1.42 (s, 9H), 0.94 (d, J = 6.5 Hz, 3H). |

### Example 139. Synthesis of tert-butyl (S)-4-(7-(5-cyano-4-methylthiazol-2-yl)-5-(2-fluorophenyl)-7H-pyrrolo[2,3-d]pyrimidin-4-yl)-3-methylpiperazine-1-carboxylate (Compound 463)

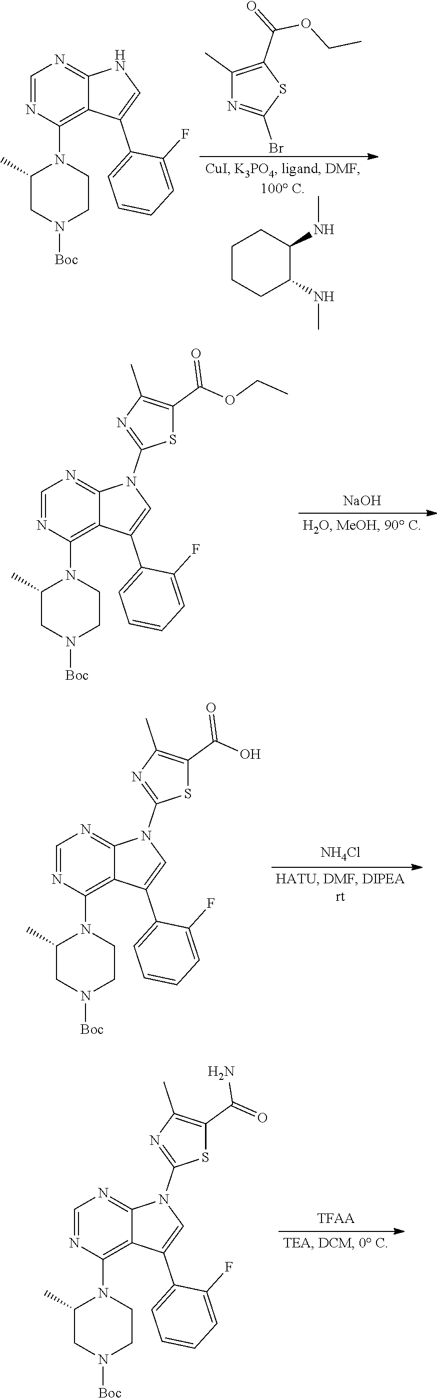

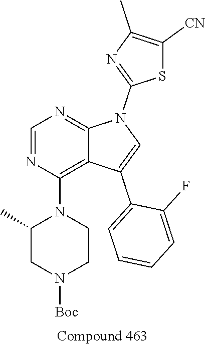 [SMILES: Cc1nc(-n2cc(-c3ccccc3F)c3c(N4CCN(C(=O)OC(C)(C)C)C[C@@H]4C)ncnc32)sc1C#N]

### Step 1. Ethyl (S)-2-(4-(4-(tert-butoxycarbonyl)-2-methylpiperazin-1-yl)-5-(2-fluorophenyl)-7H-pyrrolo[2,3-d]pyrimidin-7-yl)-4-methylthiazole-5-carboxylate

To a solution of tert-butyl (S)-4-(5-(2-fluorophenyl)-7H-pyrrolo[2,3-d]pyrimidin-4-yl)-3-methylpiperazine-1-carboxylate (500 mg, 1.2 mmol, prepared following the procedure outlined in compound 268) in DMF (5 mL) were added ethyl 2-bromo-4-methylthiazole-5-carboxylate (610 mg, 2.4 mmol), trans-N,N′-dimethylcyclohexane-1,2-diamine (350 mg, 2.4 mmol), CuI (230 mg, 1.2 mmol) and K₃PO₄ (780 mg, 3.7 mmol). The resulting mixture was heated to 100° C. overnight. After being cooling to room temperature, the reaction was partitioned between EtOAc and water and the organic layer was separated. The aqueous layer was extracted with EtOAc twice, the combined organic layers were washed with brine, dried over Na₂SO₄, filtered. and concentrated. The residue was purified by flash column chromatography (silica gel, 0˜15%, ethyl acetate in petroleum ether) to afford ethyl (S)-2-(4-(4-(tert-butoxycarbonyl)-2-methylpiperazin-1-yl)-5-(2-fluorophenyl)-7H-pyrrolo[2,3-d]pyrimidin-7-yl)-4-methylthiazole-5-carboxylate (600 mg, 84%) as a solid. LC/MS ESI (m/z): 581 (M+H)⁺.

### Step 2. (S)-2-(4-(4-(tert-Butoxycarbonyl)-2-methylpiperazin-1-yl)-5-(2-fluorophenyl)-7H-pyrrolo[2,3-d]pyrimidin-7-yl)-4-methylthiazole-5-carboxylic acid

To a solution of ethyl (S)-2-(4-(4-(tert-butoxycarbonyl)-2-methylpiperazin-1-yl)-5-(2-fluorophenyl)-7H-pyrrolo[2,3-d]pyrimidin-7-yl)-4-methylthiazole-5-carboxylate (450 mg, 0.78 mmol) in MeOH (5 mL) and H₂O (5 mL) was added NaOH (160 mg, 3.9 mmol). The resulting mixture was heated to 45° C. overnight. The reaction was cooled, adjusted to acidic pH, and filtered to afford (S)-2-(4-(4-(tert-butoxycarbonyl)-2-methylpiperazin-1-yl)-5-(2-fluorophenyl)-7H-pyrrolo[2,3-d]pyrimidin-7-yl)-4-methylthiazole-5-carboxylic acid (250 mg, 58%) as a solid. LC/MS ESI (m/z): 553 (M+H)⁺.

### Step 3. tert-Butyl (S)-4-(7-(5-carbamoyl-4-methylthiazol-2-yl)-5-(2-fluorophenyl)-7H-pyrrolo[2,3-d]pyrimidin-4-yl)-3-methylpiperazine-1-carboxylate

To a solution of (S)-2-(4-(4-(tert-butoxycarbonyl)-2-methylpiperazin-1-yl)-5-(2-fluorophenyl)-7H-pyrrolo[2,3-d]pyrimidin-7-yl)-4-methylthiazole-5-carboxylic acid (70 mg, 0.13 mmol) in DMF (5 mL) were added NH₄Cl (14 mg, 0.26 mmol), DIPEA (50 mg, 0.39 mmol) and HATU (98 mg, 0.26 mmol). The resulting mixture was stirred at room temperature overnight under N₂. The reaction was partitioned between EtOAc and water and the organic layer was separated. The aqueous layer was extracted with EtOAc twice, the combined organic layers were washed with brine, dried over Na₂SO₄, filtered, and concentrated. The residue was purified by flash column chromatography (silica gel, 0-10%, MeOH in DCM) to afford tert-butyl (S)-4-(7-(5-carbamoyl-4-methylthiazol-2-yl)-5-(2-fluorophenyl)-7H-pyrrolo[2,3-d]pyrimidin-4-yl)-3-methylpiperazine-1-carboxylate (50 mg, 71%). as a solid. LC/MS ESI (m/z): 552 (M+H)⁺.

### Step 4. tert-Butyl (S)-4-(7-(5-cyano-4-methylthiazol-2-yl)-5-(2-fluorophenyl)-7H-pyrrolo[2,3-d]pyrimidin-4-yl)-3-methylpiperazine-1-carboxylate

To a solution of tert-butyl (S)-4-(7-(5-carbamoyl-4-methylthiazol-2-yl)-5-(2-fluorophenyl)-7H-pyrrolo[2,3-d]pyrimidin-4-yl)-3-methylpiperazine-1-carboxylate (50 mg, 0.090 mmol) in DCM (3 mL) were added TEA (91 mg, 0.90 mmol) and TFAA (71 mg, 0.34 mmol) and the resulting mixture was stirred at 0° C. 1 h under N₂. Solvent was removed and the residue was purified by prep-HPLC to afford tert-butyl (S)-4-(7-(5-cyano-4-methylthiazol-2-yl)-5-(2-fluorophenyl)-7H-pyrrolo[2,3-d]pyrimidin-4-yl)-3-methylpiperazine-1-carboxylate (30 mg, 63%) of white solid. LC/MS ESI (m/z): 534 (M+H)⁺. ¹H NMR (400 MHz, CDCl₃) δ 8.57 (s, 1H), 8.06 (s, 1H), 7.47-7.34 (m, 3H), 7.21-7.19 (m, 1H), 4.46-4.17 (m, 1H), 3.85-3.69 (m, 1H), 3.63-3.56 (m, 1H), 3.55-3.46 (m, 1H), 3.07 (t, J=12.4 Hz, 1H), 2.96-2.69 (m, 2H), 2.60 (s, 3H), 1.43 (s, 9H), 1.04 (d, 3H).

### Example 140. Synthesis of tert-butyl (S)-4-(7-(4-cyanopyridin-2-yl)-5-(2-hydroxypropan-2-yl)-7H-pyrrolo[2,3-d]pyrimidin-4-yl)-3-methylpiperazine-1-carboxylate (Compound 484)

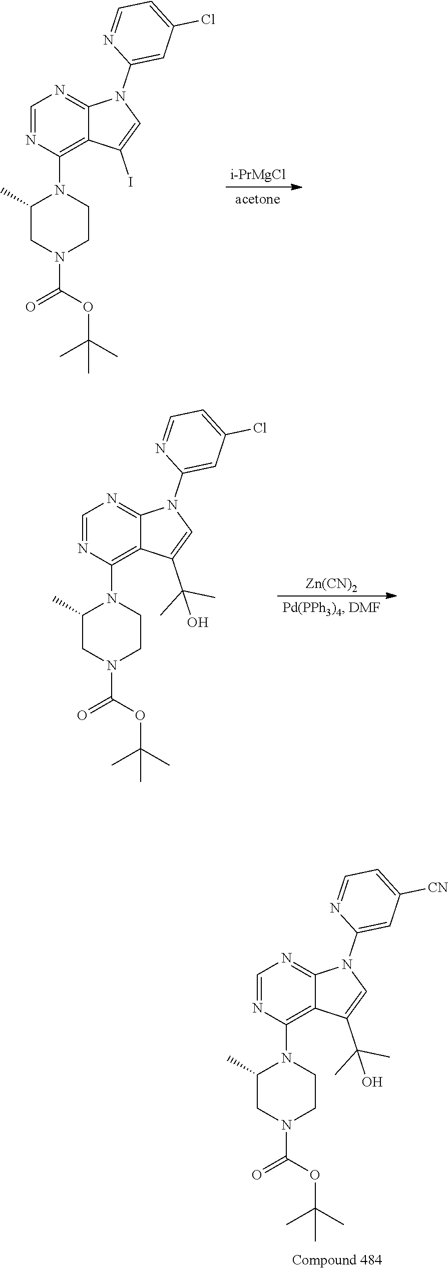

### Step 1. tert-Butyl (S)-4-(7-(4-chloropyridin-2-yl)-5-(2-hydroxypropan-2-yl)-7H-pyrrolo[2,3-d]pyrimidin-4-yl)-3-methylpiperazine-1-carboxylate

A solution of i-PrMgCl (3.6 mL, 1.0M in THF) was added dropwise to a solution of tert-butyl (S)-4-(7-(4-chloropyridin-2-yl)-5-iodo-7H-pyrrolo[2,3-d]pyrimidin-4-yl)-3-methylpiperazine-1-carboxylate (1.0 g, 1.8 mmol, prepared following the procedure outlined in Compound 486 and acetone (5 mL) in THE (20 mL) at −78° C. The reaction was allowed to warm to room temperature and stirred for 1 h. The reaction mixture was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0-30%, ethyl acetate in petroleum ether) to afford tert-butyl (S)-4-(7-(4-chloropyridin-2-yl)-5-(2-hydroxypropan-2-yl)-7H-pyrrolo[2,3-d]pyrimidin-4-yl)-3-methylpiperazine-1-carboxylate (30 mg, 3.0%) as an off white solid. LC/MS ESI (m/z): 487 (M+H)⁺.

### Step 2. tert-Butyl (S)-4-(7-(4-cyanopyridin-2-yl)-5-(2-hydroxypropan-2-yl)-7H-pyrrolo[2,3-d]pyrimidin-4-yl)-3-methylpiperazine-1-carboxylate

To a solution of tert-butyl (S)-4-(7-(4-chloropyridin-2-yl)-5-(2-hydroxypropan-2-yl)-7H-pyrrolo[2,3-d]pyrimidin-4-yl)-3-methylpiperazine-1-carboxylate (30 mg, 0.060 mmol) in DMF (5 mL) were added Zn(CN)₂ (72 mg, 0.62 mmol) and Pd(PPh₃)₄ (360 mg, 0.31 mmol). The resulting mixture was stirred at 120° C. overnight under a N₂ atmosphere. The reaction was quenched with ice water and extracted twice with EtOAc. The combined organic layers were washed with brine, dried over Na₂SO₄, filtered and concentrated. The residue was purified by flash column chromatography (silica gel, 0-20%, ethyl acetate in petroleum ether) to give crude product which was further purified by prep-HPLC to afford tert-butyl (S)-4-(7-(4-cyanopyridin-2-yl)-5-(2-hydroxypropan-2-yl)-7H-pyrrolo[2,3-d]pyrimidin-4-yl)-3-methylpiperazine-1-carboxylate (3.7 mg, 13%) as a white solid. LC/MS ESI (m/z): 478 (M+H)⁺. ¹H NMR (400 MHz, CDCl₃) δ 9.36 (s, 1H), 8.91 (s, 1H), 8.65 (d, J=5.0 Hz, 1H), 8.21 (s, 1H), 7.72 (s, 1H), 7.43 (d, J=5.1 Hz, 1H), 4.25-4.09 (m, 2H), 3.69-3.62 (m, 1H), 3.22-3.07 (m, 3H), 2.91-2.81 (m, 1H), 1.75 (s, 3H), 1.64 (s, 3H), 1.50 (s, 9H), 0.90 (d, J=6.2 Hz, 3H).

### Example 141. Synthesis of tert-butyl (S)-4-(7-(5-cyanothiazol-2-yl)-5-(pyrrolidin-1-yl)-7H-pyrrolo[2,3-d]pyrimidin-4-yl)-3-methylpiperazine-1-carboxylate (Compound 485)

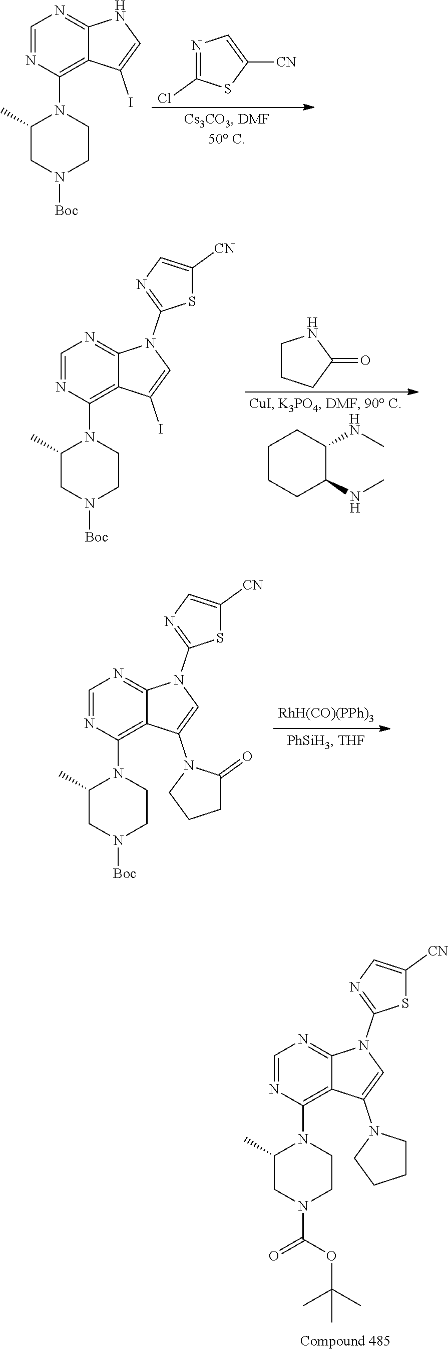

### Step 1. tert-Butyl (S)-4-(7-(5-cyanothiazol-2-yl)-5-iodo-7H-pyrrolo[2,3-d]pyrimidin-4-yl)-3-methylpiperazine-1-carboxylate

To a solution of tert-butyl (S)-4-(5-iodo-7H-pyrrolo[2,3-d]pyrimidin-4-yl)-3-methylpiperazine-1-carboxylate (600 mg, 1.4 mmol, prepared following the procedure outlined in Compound 514 in DMF (15 mL) were added 2-chlorothiazole-5-carbonitrile (390 mg, 2.7 mmol) and Cs₂CO₃ (2.2 g, 6.8 mmol). The reaction was stirred at room temperature for 3 h. The resulting mixture was filtered and the filtrate was partitioned between EtOAc and water. The aqueous layer was extracted twice with EtOAc. The combined organic layers were washed with brine, dried over Na₂SO₄, filtered and concentrated. The residue was purified by flash chromatography (silica gel, 0-30% ethyl acetate in petroleum ether) to afford tert-butyl (S)-4-(7-(5-cyanothiazol-2-yl)-5-iodo-7H-pyrrolo[2,3-d]pyrimidin-4-yl)-3-methylpiperazine-1-carboxylate (700 mg, 94%). LC/MS ESI (m/z): 552 (M+H)⁺.

### Step 2. tert-Butyl (S)-4-(7-(5-cyanothiazol-2-yl)-5-(2-oxopyrrolidin-1-yl)-7H-pyrrolo[2,3-d]pyrimidin-4-yl)-3-methylpiperazine-1-carboxylate

A mixture of tert-butyl (S)-4-(7-(5-cyanothiazol-2-yl)-5-iodo-7H-pyrrolo[2,3-d]pyrimidin-4-yl)-3-methylpiperazine-1-carboxylate (600 mg, 1.1 mmol), pyrrolidin-2-one (190 mg, 2.2 mmol), CuI (100 mg, 0.55 mmol), K₃PO₄ (690 mg, 3.3 mmol) and trans-N¹,N²-dimethylcyclohexane-1,2-diamine (160 mg, 1.1 mmol) and DMF (15 mL) was stirred at 90° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0-30%, ethyl acetate in petroleum ether) to afford tert-butyl (S)-4-(7-(5-cyanothiazol-2-yl)-5-(2-oxopyrrolidin-1-yl)-7H-pyrrolo[2,3-d]pyrimidin-4-yl)-3-methylpiperazine-1-carboxylate (130 mg, 23%) as a solid. LC/MS ESI (m/z): 509 (M+H)⁺.

### Step 3. tert-Butyl (S)-4-(7-(5-cyanothiazol-2-yl)-5-(pyrrolidin-1-yl)-7H-pyrrolo[2,3-d]pyrimidin-4-yl)-3-methylpiperazine-1-carboxylate

To a solution of tert-butyl (S)-4-(7-(5-cyanothiazol-2-yl)-5-(2-oxopyrrolidin-1-yl)-7H-pyrrolo[2,3-d]pyrimidin-4-yl)-3-methylpiperazine-1-carboxylate (40 mg, 0.080 mmol) in THF (5 mL) at 0° C. were added RhH(CO)(PPh)₃ (36 mg, 0.040 mmol) and PhSiH₃ (0.090 m1, 0.24 mmol). The resulting mixture was stirred at room temperature for 1 h. The reaction was quenched with water and extracted twice with EtOAc. The combined organic layers were washed with brine, dried over Na₂SO₄, filtered and concentrated. The residue was purified by flash column chromatography (silica gel, ethyl acetate in petroleum ether) to give crude product which was further purified by prep-HPLC to afford tert-butyl (S)-4-(7-(5-cyanothiazol-2-yl)-5-(pyrrolidin-1-yl)-7H-pyrrolo[2,3-d]pyrimidin-4-yl)-3-methylpiperazine-1-carboxylate (15 mg, 53%) as a white solid. LC/MS ESI (m/z): 495 (M+H)⁺. ¹H NMR (400 MHz, CDCl₃) δ 8.42 (s, 1H), 7.96 (s, 1H), 7.28 (s, 1H), 4.95-4.87 (m, 1H), 4.20-3.82 (m, 3H), 3.35-3.27 (m, 1H), 3.18-3.04 (m, 3H), 2.92-2.80 (m, 3H), 1.94-1.89 (m, 4H), 1.42 (s, 9H), 1.08 (d, J=6.3 Hz, 3H).

### Example 142. Synthesis of tert-butyl (S)-4-(7-(4-cyanopyridin-2-yl)-5-(ethyl(2-methoxyethyl)amino)-7H-pyrrolo[2,3-d]pyrimidin-4-yl)-3-methylpiperazine-1-carboxylate (Compound 486)

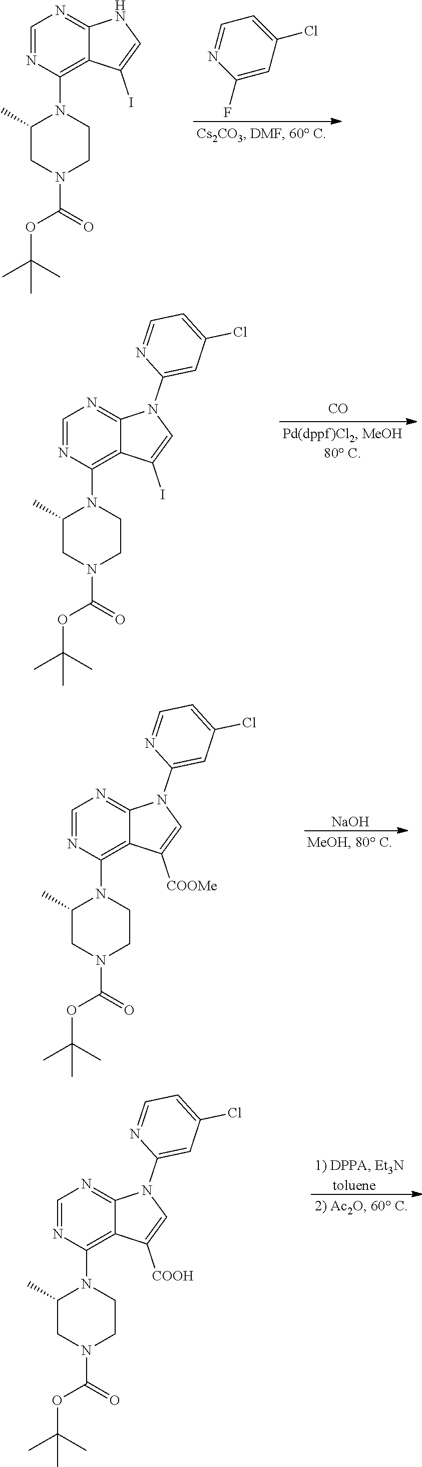

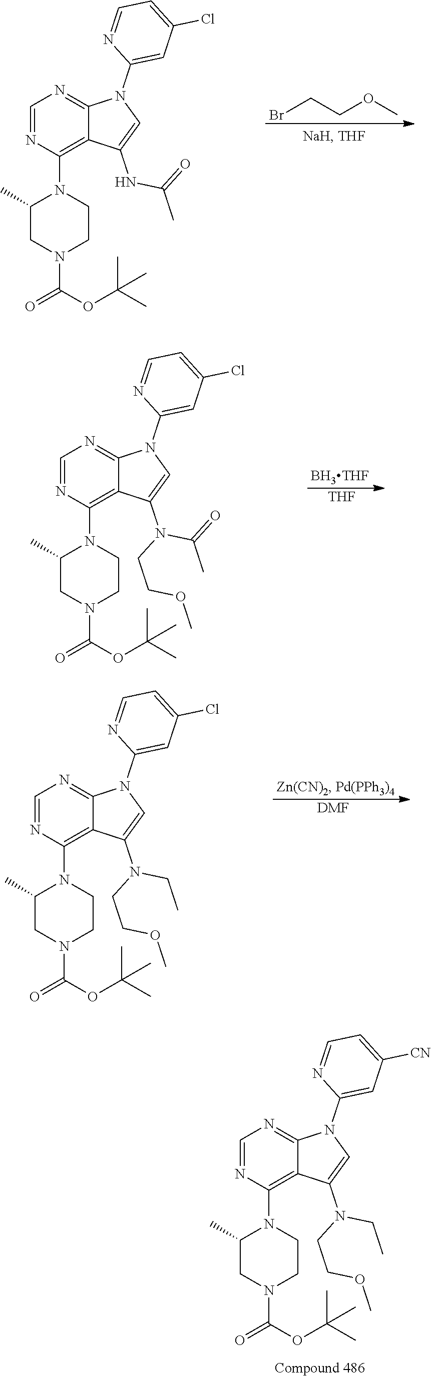

### Step 1. tert-Butyl (S)-4-(7-(4-chloropyridin-2-yl)-5-iodo-7H-pyrrolo[2,3-d]pyrimidin-4-yl)-3-methylpiperazine-1-carboxylate

To a solution of tert-butyl (S)-4-(5-iodo-7H-pyrrolo[2,3-d]pyrimidin-4-yl)-3-methylpiperazine-1-carboxylate (550 mg, 1.2 mmol) and 4-chloro-2-fluoropyridine (180 mg, 1.4 mmol) in DMF (10 mL) was added Cs₂CO₃ (780 mg, 2.4 mmol). The resulting mixture was stirred at 60° C. for 18 h. After cooling to room temperature, the reaction was partitioned between EtOAc and water. The aqueous layer was extracted twice with EtOAc. The combined organic layers were washed with brine, dried over anhydrous Na₂SO₄, filtered and concentrated. The residue was purified by column chromatography on silica gel (0˜30% EtOAc in petroleum ether) to give tert-butyl (S)-4-(7-(4-chloropyridin-2-yl)-5-iodo-7H-pyrrolo[2,3-d]pyrimidin-4-yl)-3-methylpiperazine-1-carboxylate (530 mg, 79%) as a white solid. LC/MS ESI (m/z): 555 (M+H)⁺.

### Step 2. Methyl (S)-4-(4-(tert-butoxycarbonyl)-2-methylpiperazin-1-yl)-7-(4-chloropyridin-2-yl)-7H-pyrrolo[2,3-d]pyrimidine-5-carboxylate

To a solution of tert-butyl (S)-4-(7-(4-chloropyridin-2-yl)-5-iodo-7H-pyrrolo[2,3-d]pyrimidin-4-yl)-3-methylpiperazine-1-carboxylate (530 mg, 0.94 mmol) in MeOH (6 mL) was added Pd(dppf)Cl₂ (69 mg, 0.094 mmol) at 25° C. The mixture was degassed by purging three times with CO gas. The reaction was stirred at 80° C. under CO for 8 h. The mixture was poured into H₂O (20 mL) and extracted twice with EtOAc. The combined organic phases were washed with brine, dried over Na₂SO₄, filtered and concentrated in vacuo. The residue was purified by flash chromatography (silica gel, 0-35% ethyl acetate in petroleum ether) to afford methyl (S)-4-(4-(tert-butoxycarbonyl)-2-methylpiperazin-1-yl)-7-(4-chloropyridin-2-yl)-7H-pyrrolo[2,3-d]pyrimidine-5-carboxylate (360 mg, 79%) as a brown solid. LC/MS ESI (m/z): 487 (M+H)⁺.

### Step 3. (S)-4-(4-(tert-Butoxycarbonyl)-2-methylpiperazin-1-yl)-7-(4-chloropyridin-2-yl)-7H-pyrrolo[2,3-d]pyrimidine-5-carboxylic acid

To a solution of methyl (S)-4-(4-(tert-butoxycarbonyl)-2-methylpiperazin-1-yl)-7-(4-chloropyridin-2-yl)-7H-pyrrolo[2,3-d]pyrimidine-5-carboxylate (360 mg, 0.74 mmol) in MeOH (5 mL) was added NaOH (5.0 mL, 2.0 N) at room temperature. The reaction was stirred at 80° C. for 12 h. The mixture was adjusted to pH 2 with HCl (1 N) and extracted twice with DCM. The combined organic layers were dried over Na₂SO₄, filtered and concentrated. The residue was used in next step directly.

### Step 4. tert-Butyl (S)-4-(5-acetamido-7-(4-chloropyridin-2-yl)-7H-pyrrolo[2,3-d]pyrimidin-4-yl)-3-methylpiperazine-1-carboxylate

To a solution of (S)-4-(4-(tert-butoxycarbonyl)-2-methylpiperazin-1-yl)-7-(4-chloropyridin-2-yl)-7H-pyrrolo[2,3-d]pyrimidine-5-carboxylic acid (0.74 mmol) in toluene (5 mL) were added Et₃N (320 mg, 3.2 mmol) and DPPA (0.45 mL, 2.1 mmol) at room temperature. The mixture was stirred for 2 h and then treated with acetic anhydride (320 mg, 3.2 mmol). The reaction was heated at 60° C. for 2 h. After cooling to room temperature, the mixture was poured into ice water and extracted twice with EtOAc. The combined organic phases were washed with brine, dried over Na₂SO₄, filtered and concentrated in vacuo. The crude product was purified by silica gel column (silica gel, 0-50%, ethyl acetate in petroleum ether) to afford tert-butyl (S)-4-(5-acetamido-7-(4-chloropyridin-2-yl)-7H-pyrrolo[2,3-d]pyrimidin-4-yl)-3-methylpiperazine-1-carboxylate (350 mg, 67%) as a yellow oil. LC/MS ESI (m/z): 486 (M+H)⁺.

### Step 5. tert-Butyl (S)-4-(7-(4-chloropyridin-2-yl)-5-(N-(2-methoxyethyl)acetamido)-7H-pyrrolo[2,3-d]pyrimidin-4-yl)-3-methylpiperazine-1-carboxylate

To a suspension of NaH (55 mg, 1.3 mmol, 60 wt %) in THE (10 mL) at 0° C. was added a solution of tert-butyl (S)-4-(5-acetamido-7-(4-chloropyridin-2-yl)-7H-pyrrolo[2,3-d]pyrimidin-4-yl)-3-methylpiperazine-1-carboxylate (320 mg, 0.66 mmol) in THE (5 mL) dropwise. After stirring at 0° C. for 15 min, 1-bromo-2-methoxyethane (130 mg, 0.75 mmol) was added. The resulting mixture was stirred at room temperature overnight under N₂. The reaction was quenched with ice water and extracted twice with EtOAc. The combined organic layers were washed with brine, dried over Na₂SO₄, filtered and concentrated. The residue was purified by flash column chromatography (silica gel, 0-30% ethyl acetate in petroleum ether) to afford tert-butyl (S)-4-(7-(4-chloropyridin-2-yl)-5-(N-(2-methoxyethyl)acetamido)-7H-pyrrolo[2,3-d]pyrimidin-4-yl)-3-methylpiperazine-1-carboxylate (160 mg, 45%) as a light yellow solid. LC/MS ESI (m/z): 544 (M+H)⁺.

### Step 6. tert-Butyl (S)-4-(7-(4-chloropyridin-2-yl)-5-(ethyl(2-methoxyethyl)amino)-7H-pyrrolo[2,3-d]pyrimidin-4-yl)-3-methylpiperazine-1-carboxylate

To a solution of tert-butyl (S)-4-(7-(4-chloropyridin-2-yl)-5-(N-(2-methoxyethyl)acetamido)-7H-pyrrolo[2,3-d]pyrimidin-4-yl)-3-methylpiperazine-1-carboxylate (160 mg, 0.30 mmol) in THE (2 mL) at 0° C. was added BH₃-THF (2.0 mL, 1.0 M) dropwise. After stirring at room temperature for 2 h, the reaction was quenched with ice water and extracted twice with EtOAc. The combined organic layers were washed with brine, dried over Na₂SO₄, filtered and concentrated. The residue was purified by flash column chromatography (silica gel, 0-40% ethyl acetate in petroleum ether) to afford tert-butyl (S)-4-(7-(4-chloropyridin-2-yl)-5-(ethyl(2-methoxyethyl)amino)-7H-pyrrolo[2,3-d]pyrimidin-4-yl)-3-methylpiperazine-1-carboxylate (87 mg, 55%) as a colorless oil. LC/MS ESI (m/z): 530 (M+H)⁺.

### Step 7. tert-Butyl (S)-4-(7-(4-cyanopyridin-2-yl)-5-(ethyl(2-methoxyethyl)amino)-7H-pyrrolo[2,3-d]pyrimidin-4-yl)-3-methylpiperazine-1-carboxylate

To the solution of tert-butyl (S)-4-(7-(4-chloropyridin-2-yl)-5-(ethyl(2-methoxyethyl)amino)-7H-pyrrolo[2,3-d]pyrimidin-4-yl)-3-methylpiperazine-1-carboxylate (87 mg, 0.16 mmol) in DMF (5 mL) were added Zn(CN)₂ (110 mg, 0.96 mmol) and Pd(PPh₃)₄ (92 mg, 0.080 mmol). The reaction was stirred at 120° C. under N₂ overnight. After cooling to room temperature, the reaction mixture was concentrated under reduced pressure. The residue was purified by flash column chromatography (silica gel, 0-40% ethyl acetate in petroleum ether) to give crude product which was further purified by prep-HPLC to and to afford tert-butyl (S)-4-(7-(4-cyanopyridin-2-yl)-5-(ethyl(2-methoxyethyl)amino)-7H-pyrrolo[2,3-d]pyrimidin-4-yl)-3-methylpiperazine-1-carboxylate (5.3 mg, 6.0%) as a yellow solid. LC/MS ESI (m/z): 521 (M+H)⁺. ¹H NMR (400 MHz, CD₃OD) δ 9.26 (s, 1H), 8.65 (dd, J=5.0, 0.7 Hz, 1H), 8.39 (s, 1H), 7.77 (s, 1H), 7.50 (dd, J=5.0, 1.3 Hz, 1H), 5.23 (s, 1H), 4.29 (s, 1H), 4.12 (d, J=12.5 Hz, 1H), 3.91 (d, J=13.2 Hz, 1H), 3.57-3.51 (m, 2H), 3.43-3.37 (m, 2H), 3.34-3.31 (m, 6H), 3.27-3.22 (m, 1H), 3.12 (s, 1H), 1.49 (s, 9H), 1.10 (d, J=6.4 Hz, 3H), 0.98 (t, J=7.0 Hz, 3H).

The following compound was prepared by a procedure similar to the synthesis of compound 486 from the corresponding amine (the Curtius rearrangement was run with acetic formic anhydride instead with acetic anhydride).

| Cmpd | | |
|---|---|---|
| No. | Chemical Name | LCMS and ¹H NMR |
| 499 | tert-butyl (2R,5S)-4-(7-(4- | LC/MS ESI (m/z): 505 (M + H)⁺. ¹H NMR (400 MHz, |
| | cyanopyridin-2-yl)-5- | CDCl₃) δ 9.37 (s, 1H), 8.58 (d, J = 5.0 Hz, 1H), 8.44 (s, |
| | (methyl(propyl)amino)-7H- | 1H), 7.58 (s, 1H), 7.29 (dd, J = 5.0, 1.3 Hz, 1H), 5.24 (m, |
| | pyrrolo[2,3-d]pyrimidin-4-yl)- | 1H), 4.52-4.27 (m, 1H), 4.02 (m, 1H), 3.78-3.57 (m, |
| | 2,5-dimethylpiperazine-1- | 3H), 3.19-3.11 (m, 1H), 2.89 (m, 1H), 2.76 (s, 3H), 1.73- |
| | carboxylate | 1.61 (m, 2H), 1.49 (s, 9H), 1.10 (d, J = 6.0 Hz, 6H), 0.90 (t, |
| | | J = 7.3 Hz, 3H). |

### Example 143. Synthesis of tert-butyl (S)-4-(7-(4-cyanopyridin-2-yl)-5-propoxy-7H-pyrrolo[2,3-d]pyrimidin-4-yl)-3-methylpiperazine-1-carboxylate (Compound 487)

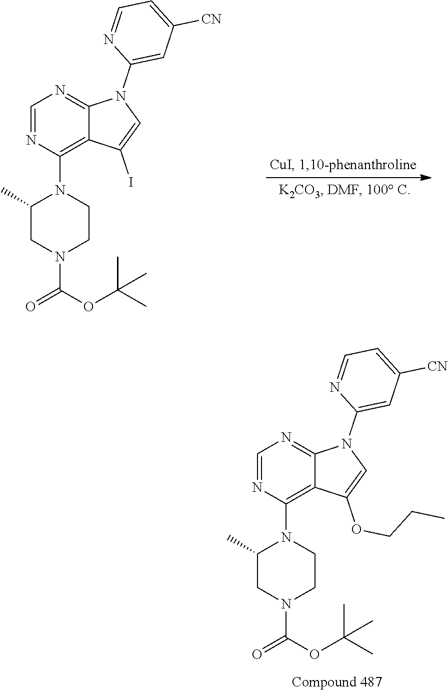 [SMILES: C[C@H]1CN(C(=O)OC(C)(C)C)CCN1c1ncnc2c1c(I)cn2-c1cc(C#N)ccn1 ; CCCOc1cn(-c2cc(C#N)ccn2)c2ncnc(N3CCN(C(=O)OC(C)(C)C)C[C@@H]3C)c12]

To the solution of tert-butyl (S)-4-(7-(4-cyanopyridin-2-yl)-5-iodo-7H-pyrrolo[2,3-d]pyrimidin-4-yl)-3-methylpiperazine-1-carboxylate (100 mg, 0.18 mmol, prepared following the procedure outlined in Compound 514 in propan-1-ol (1 mL) were added CuI (35 mg, 0.18 mmol), 1,10-phenanthroline (65 mg, 0.36 mmol) and Cs₂CO₃ (180 mg, 0.54 mmol) respectively. The resulting reaction mixtures were stirred at 100° C. under N2 overnight. This reaction was repeated at the same scale and the combined reaction mixtures were worked up together. After cooling to room temperature, solvent was removed and the residue was purified by column chromatography on silica gel (0-20% ethyl acetate in petroleum ether) and prep-HPLC to obtain tert-butyl (S)-4-(7-(4-cyanopyridin-2-yl)-5-iodo-7H-pyrrolo[2,3-d]pyrimidin-4-yl)-3-methylpiperazine-1-carboxylate (5.5 mg, 3.1%) as a white solid. LC/MS(ESI) m/z: 478 (M+H)⁺. ¹H NMR (400 MHz, CDCl₃) δ 9.38-9.36 (m, 1H), 8.55 (dd, J=5.0, 0.7 Hz, 1H), 8.40 (s, 1H), 7.54 (s, 1H), 7.28 (dd, J=5.0, 1.3 Hz, 1H), 5.00-4.84 (m, 1H), 4.51-4.12 (m, 2H), 4.01 (t, J=6.6 Hz, 2H), 3.97-3.83 (m, 1H), 3.39 (t, J=14.0 Hz, 1H), 3.22-3.00 (m, 2H), 1.92-1.87 (m, 2H), 1.50 (s, 9H), 1.29 (d, J=6.6 Hz, 3H), 1.08 (t, J=7.4 Hz, 3H).

### Example 144. Synthesis of tert-butyl (S)-4-(5-(((allyloxy)carbonyl)(2-methoxyethyl)amino)-7-(4-cyanopyridin-2-yl)-7H-pyrrolo[2,3-d]pyrimidin-4-yl)-3-methylpiperazine-1-carboxylate (Compound 488)

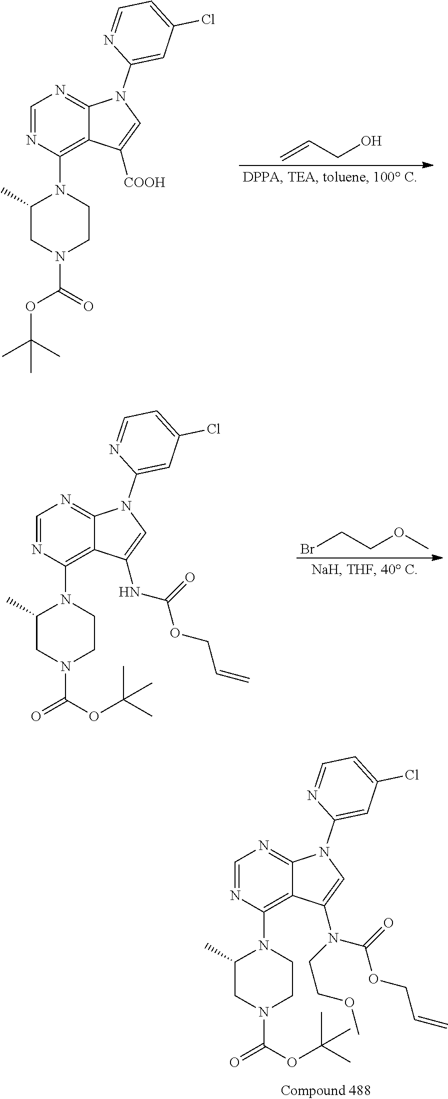

### Step 11. tert-Butyl (S)-4-(5-(((allyloxy)carbonyl)amino)-7-(4-chloropyridin-2-yl)-7H-pyrrolo[2,3-d]pyrimidin-4-yl)-3-methylpiperazine-1-carboxylate

To a solution of (S)-4-(4-(tert-butoxycarbonyl)-2-methylpiperazin-1-yl)-7-(4-chloropyridin-2-yl)-7H-pyrrolo[2,3-d]pyrimidine-5-carboxylic acid (200 mg, 0.40 mmol, prepared following the procedure outlined in Compound 486 in toluene (5 mL) were added Et₃N (0.16 mL, 1.2 mmol), prop-2-en-1-ol (0.040 mL, 0.80 mmol) and DPPA (0.16 mL, 0.80 mmol). The mixture was stirred at 100° C. overnight. After cooling to room temperature, the reaction was partitioned between EtOAc and water. The aqueous layer was extracted twice with EtOAc. The combined organic phases were washed with brine, dried over anhydrous Na₂SO₄, filtered and concentrated. The residue was purified by column chromatography on silica gel (0˜45% ethyl acetate in petroleum ether) to give the tert-butyl (S)-4-(5-(((allyloxy)carbonyl)amino)-7-(4-chloropyridin-2-yl)-7H-pyrrolo[2,3-d]pyrimidin-4-yl)-3-methylpiperazine-1-carboxylate (140 mg, 67%) as a yellow oil. LC/MS ESI (m/z): 528 (M+H)⁺.

### Step 2. tert-Butyl (S)-4-(5-(((allyloxy) carbonyl)(2-methoxyethyl)amino)-7-(4-chloropyridin-2-yl)-7H-pyrrolo[2,3-d]pyrimidin-4-yl)-3-methylpiperazine-1-carboxylate

To a suspension of NaH (24 mg, 0.52 mmol, 60 wt %) in THE (5 mL) at 0° C. was added tert-butyl (S)-4-(5-(((allyloxy)carbonyl)amino)-7-(4-chloropyridin-2-yl)-7H-pyrrolo[2,3-d]pyrimidin-4-yl)-3-methylpiperazine-1-carboxylate (140 mg, 0.26 mmol). After stirring at 0° C. for 15 min, 1-bromo-2-methoxyethane (71 mg, 0.52 mmol) was added. The resulting mixture was stirred at 40° C. overnight under N₂. The reaction was quenched with ice water and extracted twice with EtOAc. The combined organic layers were washed with brine, dried over Na₂SO₄, filtered and concentrated. The residue was purified by flash column chromatography (silica gel, 0˜20% ethyl acetate in petroleum ether) to afford tert-butyl (S)-4-(5-(((allyloxy) carbonyl)(2-methoxyethyl)amino)-7-(4-chloropyridin-2-yl)-7H-pyrrolo[2,3-d]pyrimidin-4-yl)-3-methylpiperazine-1-carboxylate (110 mg, 82%) as a light yellow solid. LC/MS ESI (m/z): 587 (M+H)⁺. ¹H NMR (400 MHz, CDCl₃) δ 9.00 (s, 1H), 8.52-8.48 (m, 1H), 8.35 (d, J=5.3 Hz, 1H), 8.13 (d, J=10.5 Hz, 1H), 7.20 (dd, J=5.3, 1.6 Hz, 1H), 5.79 (s, 1H), 5.32-4.96 (m, 2H), 4.77-4.11 (m, 5H), 3.87-3.47 (m, 5H), 3.43-3.27 (m, 5H), 3.08-2.91 (m, 1H), 1.49 (s, 9H), 1.23-1.15 (m, 3H).

The following compound was prepared by a procedure similar to the synthesis of compound 488 from the corresponding amine and alkyl halide.

| Cmpd | | |
|---|---|---|
| No. | Chemical Name | LCMS and ¹H NMR |
| 500 | tert-butyl (2R,5S)-4-(5- | LC/MS ESI (m/z): 584 (M + H)⁺. ¹H NMR (400 MHz, |
| | (((allyloxy)carbonyl)(propyl)amino)- | CDCl₃) δ 9.03 (br. s, 1H), 8.51-8.45 (m, 1H), 8.36 (d, J = |
| | 7-(4-chloropyridin-2-yl)-7H- | 5.3 Hz, 1H), 8.10-7.97 (m, 1H), 7.22-7.19 (m, 1H), 5.87 |
| | pyrrolo[2,3-d]pyrimidin-4-yl)- | (m, 1H), 5.45-5.13 (m, 2H), 4.77-4.38 (m, 4H), 4.05 (m, |
| | 2,5-dimethylpiperazine-1- | 1H), 3.72-3.49 (m, 4H), 3.09 (m, 1H), 1.60-1.51 (m, |
| | carboxylate | 2H), 1.49 (s, 9H), 1.19-1.14 (m, 3H), 1.11-1.03 (m, 3H), |
| | | 0.87 (t, J = 7.4 Hz, 3H). |

### Example 145. Synthesis of tert-butyl (2R,5S)-4-(5-(azetidin-1-yl)-7-(4-cyanopyridin-2-yl)-7H-pyrrolo[2,3-d]pyrimidin-4-yl)-2,5-dimethylpiperazine-1-carboxylate (Compound 489)

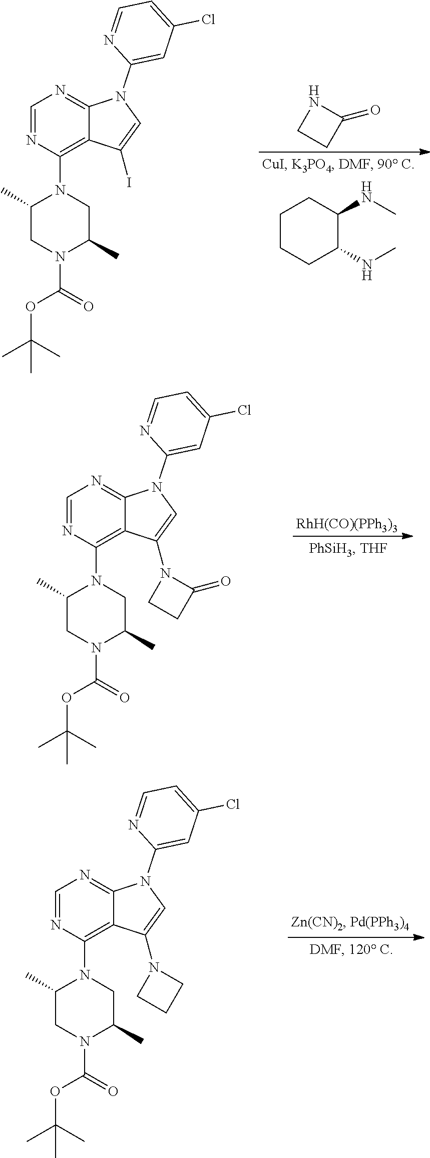

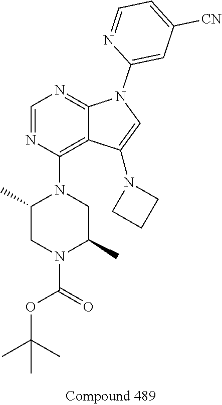 [SMILES: C[C@@H]1CN(c2ncnc3c2c(N2CCC2)cn3-c2cc(C#N)ccn2)[C@@H](C)CN1C(=O)OC(C)(C)C]

### Step 1. tert-Butyl (2R,5S)-4-(7-(4-chloropyridin-2-yl)-5-(2-oxoazetidin-1-yl)-7H-pyrrolo[2,3-d]pyrimidin-4-yl)-2,5-dimethylpiperazine-1-carboxylate

To a solution of tert-butyl (2R,5S)-4-(7-(4-chloropyridin-2-yl)-5-iodo-7H-pyrrolo[2,3-d]pyrimidin-4-yl)-2,5-dimethylpiperazine-1-carboxylate (1.0 g, 1.7 mmol, prepared following the procedure outlined in Compound 521 in DMF (25 mL) were added azetidin-2-one (310 mg, 4.2 mmol), CuI (170 mg, 0.80 mmol), trans-N¹,N²-dimethylcyclohexane-1,2-diamine (250 mg, 1.7 mmol) and K₃PO₄ (1.2 g, 5.1 mmol). The resulting mixture was heated to 90° C. overnight. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0˜80%, ethyl acetate in petroleum ether) to afford tert-butyl (2R,5S)-4-(7-(4-chloropyridin-2-yl)-5-(2-oxoazetidin-1-yl)-7H-pyrrolo[2,3-d]pyrimidin-4-yl)-2,5-dimethylpiperazine-1-carboxylate (500 mg, 57%) as a yellow solid. LC/MS ESI (m/z): 512 (M+H)⁺.

### Step 2. tert-Butyl (2R,5S)-4-(5-(azetidin-1-yl)-7-(4-chloropyridin-2-yl)-7H-pyrrolo[2,3-d]pyrimidin-4-yl)-2,5-dimethylpiperazine-1-carboxylate

To a solution of tert-butyl (2R,5S)-4-(7-(4-chloropyridin-2-yl)-5-(2-oxoazetidin-1-yl)-7H-pyrrolo[2,3-d]pyrimidin-4-yl)-2,5-dimethylpiperazine-1-carboxylate (350 mg, 0.68 mmol) in THE (10 mL) at 0° C. was added RhH(CO)(PPh)₃ (180 mg, 0.20 mmol), followed by PhSiH₃ (0.30 ml, 2.7 mmol) dropwise. The resulting mixture was stirred at room temperature for 1 h. The reaction was quenched with water and extracted twice with EtOAc. The combined organic layers were washed with brine, dried over Na₂SO₄, filtered and concentrated. The residue was purified by flash column chromatography (silica gel, 0˜40% ethyl acetate in petroleum ether) to give the tert-butyl (2R,5S)-4-(5-(azetidin-1-yl)-7-(4-chloropyridin-2-yl)-7H-pyrrolo[2,3-d]pyrimidin-4-yl)-2,5-dimethylpiperazine-1-carboxylate (200 mg, 59%) as a yellow solid. LC/MS ESI (m/z): 498 (M+H)⁺.

### Step 3. tert-Butyl (2R,5S)-4-(5-(azetidin-1-yl)-7-(4-cyanopyridin-2-yl)-7H-pyrrolo[2,3-d]pyrimidin-4-yl)-2,5-dimethylpiperazine-1-carboxylate

To the solution of tert-butyl (2R,5S)-4-(5-(azetidin-1-yl)-7-(4-chloropyridin-2-yl)-7H-pyrrolo[2,3-d]pyrimidin-4-yl)-2,5-dimethylpiperazine-1-carboxylate (200 mg, 0.40 mmol) in DMF (10 mL) were added Zn(CN)₂ (190 mg, 1.6 mmol) and Pd(PPh₃)₄ (230 mg, 0.20 mmol) respectively. The resulting mixture was stirred at 120° C. under N₂ overnight. After cooling to room temperature, the reaction mixture was concentrated under reduced pressure. The residue was purified by flash column chromatography (silica gel, 0˜40% ethyl acetate in petroleum ether) to give crude product which was further purified by prep-HPLC to afford tert-butyl (2R,5S)-4-(5-(azetidin-1-yl)-7-(4-cyanopyridin-2-yl)-7H-pyrrolo[2,3-d]pyrimidin-4-yl)-2,5-dimethylpiperazine-1-carboxylate (120 mg, 61%) as a yellow solid. LC/MS ESI (m/z): 489 (M+H)⁺. ¹H NMR (400 MHz, CDCl₃) δ 9.35 (s, 1H), 8.57 (dd, J=5.0, 0.8 Hz, 1H), 8.42 (s, 1H), 7.45 (s, 1H), 7.29 (dd, J=5.0, 1.3 Hz, 1H), 5.11 (br. s, 1H), 4.36 (m, 1H), 4.02 (d, J=13.9 Hz, 1H), 3.86 (q, J=7.0 Hz, 2H), 3.80-3.71 (m, 2H), 3.66-3.57 (m, 3H), 2.33-2.26 (m, 2H), 1.49 (s, 9H), 1.16 (d, J=6.8 Hz, 3H), 0.99 (d, J=6.8 Hz, 3H).

### Example 146. tert-Butyl (2R,5S)-4-(7-(3-chlorophenyl)-5-(trifluoromethyl)-7H-pyrrolo[2,3-d]pyrimidin-4-yl)-2,5-dimethylpiperazine-1-carboxylate (Compound 490)

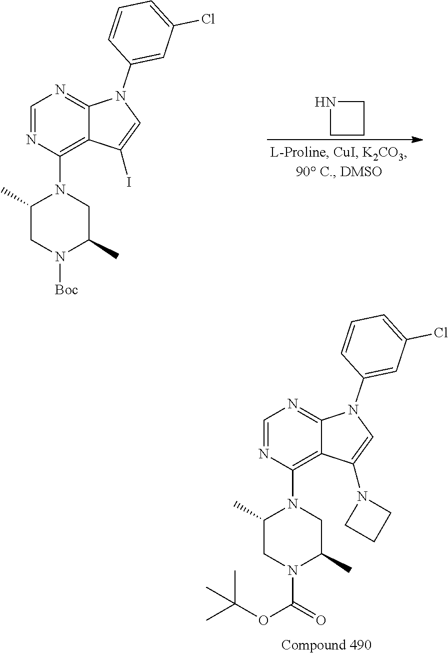

To a solution of tert-butyl (2R,5S)-4-(7-(3-chlorophenyl)-5-iodo-7H-pyrrolo[2,3-d]pyrimidin-4-yl)-2,5-dimethylpiperazine-1-carboxylate (200 mg, 0.35 mmol, prepared following the procedure outlined in Compound 491 in DMSO (3 mL) were added azetidine (40 mg, 0.70 mmol), L-Proline (40 mg, 0.37 mmol), CuI (67 mg, 0.35 mmol) and K₂CO₃ (150 mg, 1.1 mmol). The resulting mixture was heated to 90° C. overnight. After cooling to room temperature. The reaction was quenched with water and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford tert-butyl (2R,5S)-4-(5-(azetidin-1-yl)-7-(3-chlorophenyl)-7H-pyrrolo[2,3-d]pyrimidin-4-yl)-2,5-dimethylpiperazine-1-carboxylate (13 mg, 7.4%) as a white solid. LC/MS ESI (m/z): 497 (M+H)⁺. ¹H NMR (400 MHz, CDCl₃) δ 8.36 (s, 1H), 7.78-7.65 (m, 2H), 7.42 (t, J=8.1 Hz, 1H), 7.28 (s, 1H), 6.61 (s, 1H), 5.12 (s, 1H), 4.43 (s, 1H), 4.00 (d, J 13.4 Hz, 1H), 3.84-3.70 (m, 4H), 3.67-3.56 (m, 3H), 2.33-2.24 (m, 2H), 1.49 (s, 9H), 1.14 (d, J=6.7 Hz, 3H), 1.03 (d, J=6.8 Hz, 3H).

### Example 147. tert-Butyl (2R,5S)-4-(7-(3-chlorophenyl)-5-(trifluoromethyl)-7H-pyrrolo[2,3-d]pyrimidin-4-yl)-2,5-dimethylpiperazine-1-carboxylate (Compound 491)

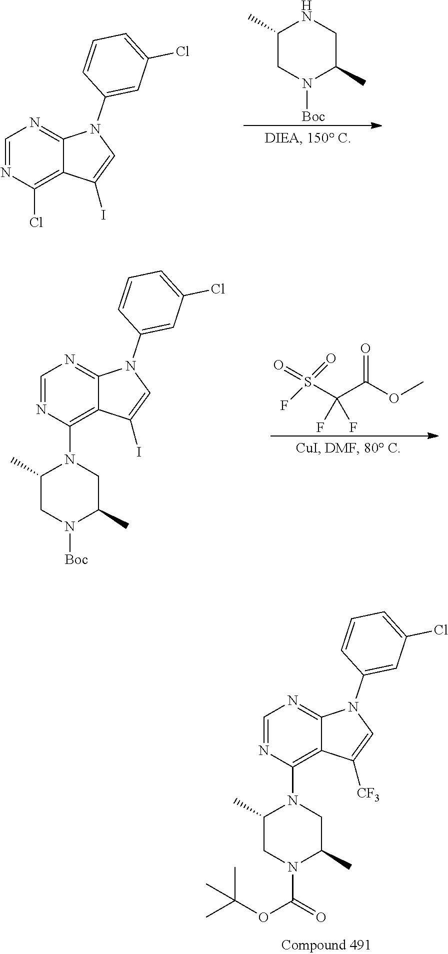

### Step 1. tert-Butyl (2R,5S)-4-(7-(3-chlorophenyl)-5-iodo-7H-pyrrolo[2,3-d]pyrimidin-4-yl)-2,5-dimethylpiperazine-1-carboxylate

To a solution of 4-chloro-7-(3-chlorophenyl)-5-iodo-7H-pyrrolo[2,3-d]pyrimidine (5.0 g, 13 mmol, prepared following the procedure outlined in Compound 522 in DIPEA (60 mL) was added tert-butyl (2R,5S)-2,5-dimethylpiperazine-1-carboxylate (5.5 g, 26 mmol). The resulting mixture was heated to 150° C. for 1 h. After cooling to room temperature, solvent was removed and the residue was purified by flash column chromatography (silica gel, 0˜30%, ethyl acetate in petroleum ether) to afford tert-butyl (2R,5S)-4-(7-(3-chlorophenyl)-5-iodo-7H-pyrrolo[2,3-d]pyrimidin-4-yl)-2,5-dimethylpiperazine-1-carboxylate (7.0 g, 96%) as a white solid. LC/MS ESI (m/z): 568 (M+H)⁺.

### Step 2. tert-Butyl (2R,5S)-4-(7-(3-chlorophenyl)-5-(trifluoromethyl)-7H-pyrrolo[2,3-d]pyrimidin-4-yl)-2,5-dimethylpiperazine-1-carboxylate

To a solution of tert-butyl (2R,5S)-4-(7-(3-chlorophenyl)-5-iodo-7H-pyrrolo[2,3-d]pyrimidin-4-yl)-2,5-dimethylpiperazine-1-carboxylate (1.0 g, 1.8 mmol) in DMF (15 mL) were added CuI (340 mg, 1.8 mmol) and methyl 2,2-difluoro-2-(fluorosulfonyl)acetate (690 mg, 3.6 mmol). The resulting mixture was stirred at 80° C. overnight. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to give tert-butyl (2R,5S)-4-(7-(3-chlorophenyl)-5-(trifluoromethyl)-7H-pyrrolo[2,3-d]pyrimidin-4-yl)-2,5-dimethylpiperazine-1-carboxylate (600 mg, 67%) as a white solid. LC/MS ESI (m/z): 510 (M+H)⁺. ¹H NMR (400 MHz, CDCl₃) δ 8.51 (s, 1H), 7.72-7.68 (m, 2H), 7.58 (d, J=7.2 Hz, 1H), 7.49 (t, J=7.5 Hz, 1H), 7.42 (d, J=7.1 Hz, 1H), 4.63-4.54 (m, 1H), 4.48-4.26 (m, 1H), 3.80-3.71 (m, 2H), 3.64 (d, J=13.6 Hz, 1H), 3.49 (d, J=13.3 Hz, 1H), 1.50 (s, 9H), 1.17-1.10 (m, 6H).

### Example 148. Synthesis of tert-butyl (S)-4-(5-(azetidin-1-yl)-7-(3-chlorophenyl)-7H-pyrrolo[2,3-d]pyrimidin-4-yl)-3-methylpiperazine-1-carboxylate (Compound 493)

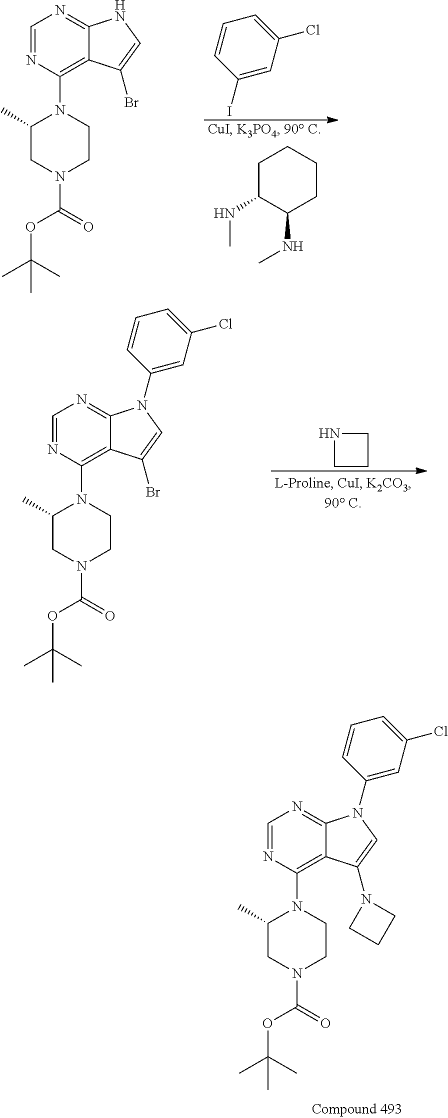

### Step 1. tert-Butyl (S)-4-(5-bromo-7-(3-chlorophenyl)-7H-pyrrolo[2,3-d]pyrimidin-4-yl)-3-methylpiperazine-1-carboxylate

To a solution of tert-butyl (S)-4-(5-bromo-7H-pyrrolo[2,3-d]pyrimidin-4-yl)-3-methylpiperazine-1-carboxylate (500 mg, 1.3 mmol, prepared following the procedure outlined in Compound 498 in DMF (10 mL) were added 1-chloro-3-iodobenzene (600 mg, 2.5 mmol), CuI (120 mg, 0.63 mmol), trans-N¹,N²-dimethylcyclohexane-1,2-diamine (180 mg, 1.3 mmol) and K₃PO₄ (800 mg, 3.8 mmol). The resulting mixture was heated to 90° C. overnight. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0˜15%, ethyl acetate in petroleum ether) to afford tert-butyl (S)-4-(5-bromo-7-(3-chlorophenyl)-7H-pyrrolo[2,3-d]pyrimidin-4-yl)-3-methylpiperazine-1-carboxylate (400 mg, 62%) as a white solid. LC/MS ESI (m/z): 506, 508 (M+H)⁺.

### Step 2. tert-Butyl (S)-4-(5-(azetidin-1-yl)-7-(3-chlorophenyl)-7H-pyrrolo[2,3-d]pyrimidin-4-yl)-3-methylpiperazine-1-carboxylate

To a solution of tert-butyl (S)-4-(5-bromo-7-(3-chlorophenyl)-7H-pyrrolo[2,3-d]pyrimidin-4-yl)-3-methylpiperazine-1-carboxylate (400 mg, 0.79 mmol) in DMSO (10 mL) were added azetidine (91 mg, 1.6 mmol), CuI (150 mg, 0.79 mmol), L-Proline (46 mg, 0.40 mmol) and K₂CO₃ (330 mg, 2.4 mmol). The resulting mixture was heated to 90° C. overnight. After cooling to room temperature. The reaction was quenched with water and extracted twice with EtOAc. The combined organic layers were washed with brine, dried over Na₂SO₄, filtered and concentrated. The residue was purified by flash column chromatography (silica gel, ethyl acetate in petroleum ether) to give crude product which was further purified by prep-HPLC to afford tert-butyl (S)-4-(5-(azetidin-1-yl)-7-(3-chlorophenyl)-7H-pyrrolo[2,3-d]pyrimidin-4-yl)-3-methylpiperazine-1-carboxylate (100 mg, 38%) as a white solid. LC/MS ESI (m/z): 483 (M+H)⁺. ¹H NMR (400 MHz, CDCl₃) δ 8.30 (s, 1H), 7.67 (s, 1H), 7.61 (d, J=8.2 Hz, 1H), 7.37-7.32 (m, 1H), 7.21 (s, 1H), 6.54 (s, 1H), 4.94-4.85 (m, 1H), 4.12-3.82 (m, 3H), 3.74-3.68 (m, 2H), 3.58-3.52 (m, 2H), 3.48-3.31 (m, 2H), 2.99-2.84 (m, 1H), 2.25-2.18 (m, 2H), 1.43 (s, 9H), 1.09 (d, J=6.7 Hz, 3H).

### Example 149. Synthesis of tert-butyl (2R,5S)-4-(7-(3-fluorophenyl)-5-(trifluoromethyl)-7H-pyrrolo[2,3-d]pyrimidin-4-yl)-2,5-dimethylpiperazine-1-carboxylate (Compound 496)

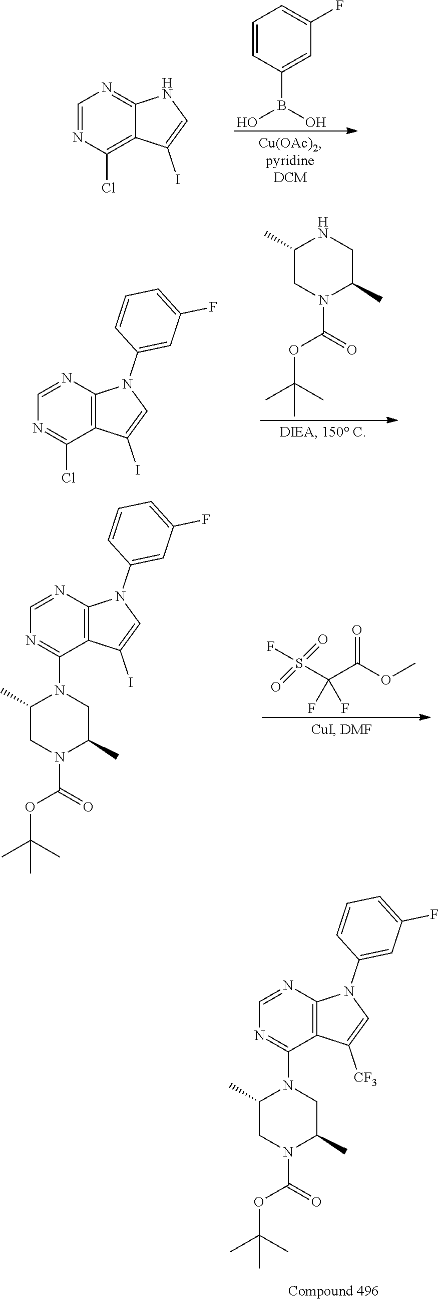

### Step 1. 4-Chloro-7-(3-fluorophenyl)-5-iodo-7H-pyrrolo[2,3-d]pyrimidine

To a solution of 4-chloro-5-iodo-7H-pyrrolo[2,3-d]pyrimidine (5.0 g, 18 mmol) in DCM (80 mL) were added (3-fluorophenyl)boronic acid (5.0 g, 36 mmol), 4 A molecular sieves (2.5 g), Cu(OAc)₂ (8.1 g, 90 mmol) and pyridine (17 g, 45 mmol). The resulting mixture was stirred at 35° C. under O₂ atmosphere overnight. The reaction was quenched with aqueous NH₄OH (30 mL) at 0° C. in an ice water bath, filtered and extracted twice with DCM. The combined organic layers were dried over Na₂SO₄, filtered and concentrated. The residue was purified by flash chromatography (silica gel, EtOAc in petroleum ether) to afford 4-chloro-7-(3-fluorophenyl)-5-iodo-7H-pyrrolo[2,3-d]pyrimidine (3.0 g, 45%) as a light yellow solid. LC/MS ESI (m/z): 374 (M+H)⁺.

### Step 2. tert-Butyl (2R,5S)-4-(7-(3-fluorophenyl)-5-iodo-7H-pyrrolo[2,3-d]pyrimidin-4-yl)-2,5-dimethylpiperazine-1-carboxylate

A mixture of 4-chloro-7-(3-fluorophenyl)-5-iodo-7H-pyrrolo[2,3-d]pyrimidine (400 mg, 1.1 mmol), tert-butyl (2R,5S)-2,5-dimethylpiperazine-1-carboxylate (340 mg, 1.6 mmol) in DIPEA (8 mL) was heated at 150° C. for 2 h. The reaction mixture was concentrated to a residue and purified by column chromatography on silica gel to give the tert-butyl (2R,5S)-4-(7-(3-fluorophenyl)-5-iodo-7H-pyrrolo[2,3-d]pyrimidin-4-yl)-2,5-dimethylpiperazine-1-carboxylate (470 mg, 79%) as a solid. LC/MS ESI (m/z): 552 (M+H)⁺.

### Step 3. tert-Butyl (2R,5S)-4-(7-(3-fluorophenyl)-5-(trifluoromethyl)-7H-pyrrolo[2,3-d]pyrimidin-4-yl)-2,5-dimethylpiperazine-1-carboxylate

A mixture of tert-butyl (2R,5S)-4-(7-(3-fluorophenyl)-5-iodo-7H-pyrrolo[2,3-d]pyrimidin-4-yl)-2,5-dimethylpiperazine-1-carboxylate (100 mg, 0.18 mmol), CuI (35 mg, 0.18 mmol) and methyl 2,2-difluoro-2-(fluorosulfonyl)acetate (140 mg, 0.73 mmol) in DMF (5 mL) was stirred at 80° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by column chromatography on silica gel to give the crude product, which was purified by prep-HPLC to give the tert-butyl (2R,5S)-4-(7-(3-fluorophenyl)-5-(trifluoromethyl)-7H-pyrrolo[2,3-d]pyrimidin-4-yl)-2,5-dimethylpiperazine-1-carboxylate (50 mg, 56%) as a white solid. LC/MS ESI (m/z): 494 (M+H)⁺. ¹H NMR (400 MHz, CDCl₃) δ 8.52 (s, 1H), 7.73 (s, 1H), 7.55-7.44 (m, 3H), 7.19-7.12 (m, 1H), 4.64-4.53 (m, 1H), 4.51-4.25 (m, 1H), 3.80-3.62 (m, 3H), 3.52-3.47 (m, 1H), 1.50 (s, 9H), 1.17-1.11 (m, 6H).

The following compound was prepared by a procedure similar to the synthesis of compound 496 from the corresponding aryl halide.

| Cmpd | | |
|---|---|---|
| No. | Chemical Name | LCMS and ¹H NMR |
| 492 | tert-butyl (2R,5S)-4-(7-(3,5- | LC/MS ESI (m/z): 512 (M + H)⁺. ¹H NMR (400 MHz, |
| | difluorophenyl)-5- | CDCl₃) δ 8.45 (s, 1H), 7.64 (s, 1H), 7.30-7.25 (m, 2H), |
| | (trifluoromethyl)-7H- | 6.85-6.79 (m, 1H), 4.56-4.48 (m, 1H), 4.44-4.21 (m, |
| | pyrrolo[2,3-d]pyrimidin-4-yl)- | 1H), 3.74-3.64 (m, 2H), 3.59-3.53 (m, 1H), 3.44-3.39 |
| | 2,5-dimethylpiperazine-1- | (m, 1H), 1.42 (s, 9H), 1.08 (d, J = 6.6 Hz, 3H), 1.05 (d, J = |
| | carboxylate | 6.8 Hz, 3H). |

### Example 150. Synthesis of tert-butyl (2R,5S)-4-(5-(azetidin-1-yl)-7-(3,5-difluorophenyl)-7H-pyrrolo[2,3-d]pyrimidin-4-yl)-2,5-dimethylpiperazine-1-carboxylate (Compound 497)

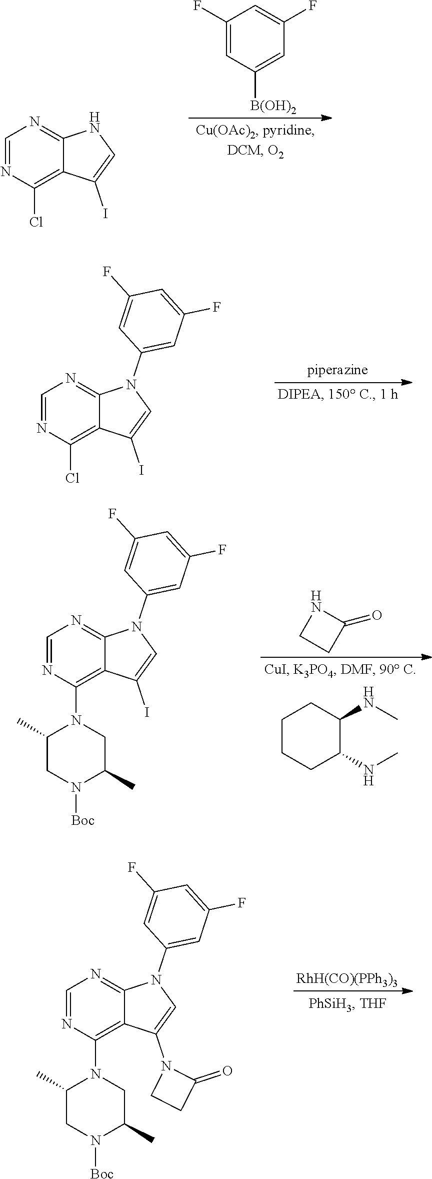

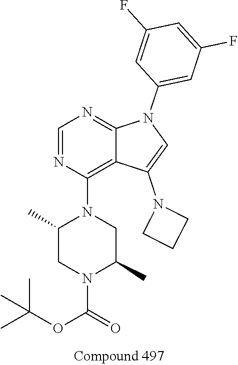 [SMILES: C[C@@H]1CN(c2ncnc3c2c(N2CCC2)cn3-c2cc(F)cc(F)c2)[C@@H](C)CN1C(=O)OC(C)(C)C]

### Step 1. 4-Chloro-7-(3,5-difluorophenyl)-5-iodo-7H-pyrrolo[2,3-d]pyrimidine

To a solution of 4-chloro-5-iodo-7H-pyrrolo[2,3-d]pyrimidine (10 g, 36 mmol) in DCM (150 mL) were added (3,5-difluorophenyl)boronic acid (11 g, 72 mmol), 4 A molecular sieves (5 g), Cu(OAc)₂ (16 g, 90 mmol) and pyridine (17 g, 210 mmol). The resulting mixture was stirred at 35° C. under O₂ atmosphere overnight. The reaction was quenched with aqueous NH₄OH (60 mL) at 0° C. in an ice water bath, filtered and extracted twice with DCM. The combined organic layers were dried over Na₂SO₄, filtered and concentrated. The residue was purified by flash chromatography (silica gel, EtOAc in petroleum ether) to afford 4-chloro-7-(3,5-difluorophenyl)-5-iodo-7H-pyrrolo[2,3-d]pyrimidine (6.7 g, 48%) as a light yellow solid. LC/MS ESI (m/z): 392 (M+H)⁺.

### Step 2. tert-Butyl (2R,5S)-4-(7-(3,5-difluorophenyl)-5-iodo-7H-pyrrolo[2,3-d]pyrimidin-4-yl)-2,5-dimethylpiperazine-1-carboxylate

To the solution of 4-chloro-7-(3,5-difluorophenyl)-5-iodo-7H-pyrrolo[2,3-d]pyrimidine (3.0 g, 7.6 mmol) in DIPEA (20 m1) was added tert-butyl (2R,5S)-2,5-dimethylpiperazine-1-carboxylate (3.2 g, 15 mmol), The resulting reaction mixture was stirred at 150° C. under N₂ for 1 h. After removal of solvent, the crude product was purified by flash column chromatography (silica gel, ethyl acetate in petroleum ether) to afford tert-butyl (2R,5S)-4-(7-(3,5-difluorophenyl)-5-iodo-7H-pyrrolo[2,3-d]pyrimidin-4-yl)-2,5-dimethylpiperazine-1-carboxylate (3.7 g, 86%) as a solid. LC/MS ESI (m/z): 570 (M+H)⁺.

### Step 3. tert-Butyl (2R,5S)-4-(7-(3,5-difluorophenyl)-5-(2-oxoazetidin-1-yl)-7H-pyrrolo[2,3-d]pyrimidin-4-yl)-2,5-dimethylpiperazine-1-carboxylate

To a solution of tert-butyl (2R,5S)-4-(7-(3,5-difluorophenyl)-5-iodo-7H-pyrrolo[2,3-d]pyrimidin-4-yl)-2,5-dimethylpiperazine-1-carboxylate (1.0 g, 1.7 mmol) in DMF (10 mL) were added azetidin-2-one (310 mg, 4.2 mmol), CuI (170 mg, 0.80 mmol), trans-N¹,N²-dimethylcyclohexane-1,2-diamine (250 mg, 1.7 mmol) and K₃PO₄ (1.2 g, 5.1 mmol). The resulting mixture was heated to 90° C. overnight. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0-65%, ethyl acetate in petroleum ether) to afford tert-butyl (2R,5S)-4-(7-(3,5-difluorophenyl)-5-(2-oxoazetidin-1-yl)-7H-pyrrolo[2,3-d]pyrimidin-4-yl)-2,5-dimethylpiperazine-1-carboxylate (420 mg, 48%) as a light yellow solid. LC/MS ESI (m/z): 513 (M+H)⁺.

### Step 4. tert-Butyl (2R,5S)-4-(5-(azetidin-1-yl)-7-(3,5-difluorophenyl)-7H-pyrrolo[2,3-d]pyrimidin-4-yl)-2,5-dimethylpiperazine-1-carboxylate

To a solution of tert-butyl (2R,5S)-4-(7-(3,5-difluorophenyl)-5-(2-oxoazetidin-1-yl)-7H-pyrrolo[2,3-d]pyrimidin-4-yl)-2,5-dimethylpiperazine-1-carboxylate (370 mg, 0.71 mmol) in THE (6 mL) at 0° C. was added RhH(CO)(PPh)₃ (340 mg, 0.30 mmol), followed PhSiH₃ (0.40 ml, 3.5 mmol) dropwise. After stirring at room temperature for 1 h, the reaction was quenched with water and extracted twice with EtOAc. The combined organic layers were washed with brine, dried over Na₂SO₄, filtered and concentrated. The residue was purified by flash column chromatography (silica gel, ethyl acetate in petroleum ether) to give a crude product, which was further purified by prep-HPLC to afford tert-butyl (2R,5S)-4-(5-(azetidin-1-yl)-7-(3,5-difluorophenyl)-7H-pyrrolo[2,3-d]pyrimidin-4-yl)-2,5-dimethylpiperazine-1-carboxylate (75 mg, 21%) as a white solid. LC/MS ESI (m/z): 499 (M+H)⁺. ¹H NMR (400 MHz, CDCl₃) δ 8.37 (s, 1H), 7.47-7.40 (m, 2H), 6.77-6.67 (m, 1H), 6.58 (s, 1H), 5.11 (s, 1H), 4.37 (m, 1H), 4.00 (d, J=13.6 Hz, 1H), 3.85-3.71 (m, 4H), 3.67-3.55 (m, 3H), 2.30 (m, 2H), 1.49 (s, 9H), 1.15 (d, J=6.7 Hz, 3H), 1.01 (d, J=6.8 Hz, 3H).

### Example 151. Synthesis of tert-butyl (S)-4-(5-(azetidin-1-yl)-7-(3,5-difluorophenyl)-7H-pyrrolo[2,3-d]pyrimidin-4-yl)-3-methylpiperazine-1-carboxylate (Compound 498)

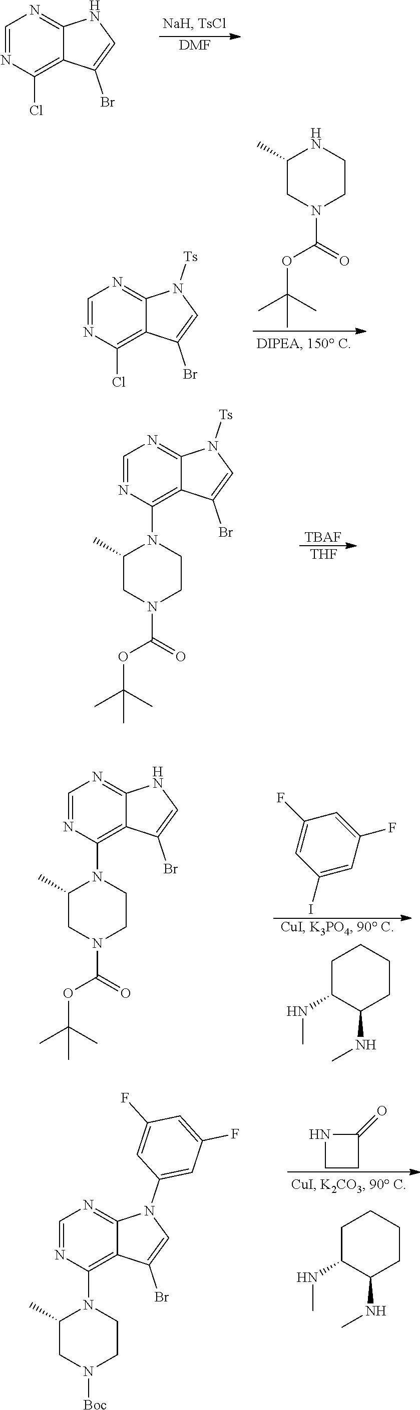

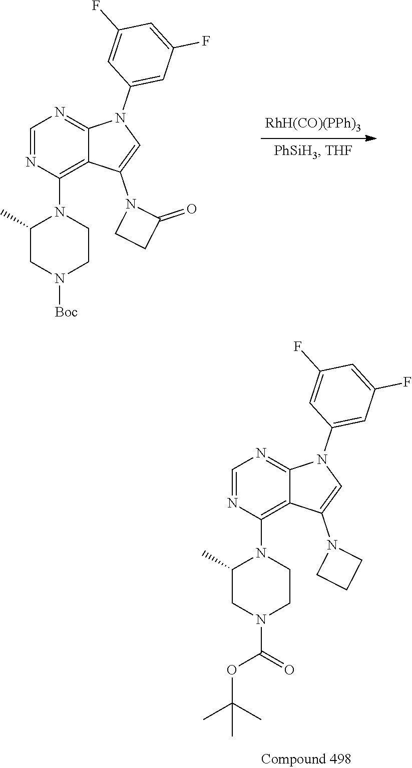 [SMILES: C[C@H]1CN(C(=O)OC(C)(C)C)CCN1c1ncnc2c1c(N1CCC1=O)cn2-c1cc(F)cc(F)c1 ; C[C@H]1CN(C(=O)OC(C)(C)C)CCN1c1ncnc2c1c(N1CCC1)cn2-c1cc(F)cc(F)c1]

### Step 1. 5-Bromo-4-chloro-7-tosyl-7H-pyrrolo[2,3-d]pyrimidine

To a suspension of NaH (5.0 g, 130 mmol, 60 wt %) in anhydrous DMF (125 mL) at 0° C. was added 5-bromo-4-chloro-7H-pyrrolo[2,3-d]pyrimidine (25 g, 110 mmol) in portions. The resulting mixture was stirred at 0° C. for 30 minutes and then treated with 4-methylbenzenesulfonyl chloride (25 g, 130 mmol) in portions. The reaction was stirred at room temperature overnight and then quenched by pouring into ice water. The precipitate was collected by filtration and dried in vacuo to provide 5-bromo-4-chloro-7-tosyl-7H-pyrrolo[2,3-d]pyrimidine (27 g, 65%) as a white solid. LC/MS ESI (m/z): 386, 388 (M+H)⁺.

### Step 2. tert-Butyl (S)-4-(5-bromo-7-tosyl-7H-pyrrolo[2,3-d]pyrimidin-4-yl)-3-methylpiperazine-1-carboxylate

To a solution of 5-bromo-4-chloro-7-tosyl-7H-pyrrolo[2,3-d]pyrimidine (17 g, 43 mmol) in DIPEA (30 mL) was added tert-butyl (S)-3-methylpiperazine-1-carboxylate (22 g, 110 mmol). The resulting mixture was stirred at 150° C. for 2.5 h. After cooling to room temperature, solvent was removed and the residue was purified by flash column chromatography (silica gel, 0˜20%, ethyl acetate in petroleum ether) to afford tert-butyl (S)-4-(5-bromo-7-tosyl-7H-pyrrolo[2,3-d]pyrimidin-4-yl)-3-methylpiperazine-1-carboxylate (20 g, 86%) as a white solid. LC/MS ESI (m/z): 550, 552 (M+H)⁺.

### Step 3. tert-Butyl (S)-4-(5-bromo-7H-pyrrolo[2,3-d]pyrimidin-4-yl)-3-methylpiperazine-1-carboxylate

To a solution of tert-butyl (S)-4-(5-bromo-7-tosyl-7H-pyrrolo[2,3-d]pyrimidin-4-yl)-3-methylpiperazine-1-carboxylate (15 g, 27 mmol) in THE (30 mL) was added TBAF (64 mL, 1.0M in THF). The resulting mixture was stirred at room temperature for 4 h. The reaction was quenched with water and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford tert-butyl (S)-4-(5-bromo-7H-pyrrolo[2,3-d]pyrimidin-4-yl)-3-methylpiperazine-1-carboxylate (9.3 g, 86%) as a white solid. LC/MS ESI (m/z): 396, 398 (M+H)⁺.

### Step 4. tert-Butyl (S)-4-(5-bromo-7-(3,5-difluorophenyl)-7H-pyrrolo[2,3-d]pyrimidin-4-yl)-3-methylpiperazine-1-carboxylate

To a solution of tert-butyl (S)-4-(5-bromo-7H-pyrrolo[2,3-d]pyrimidin-4-yl)-3-methylpiperazine-1-carboxylate (500 mg, 1.3 mmol) in DMF (10 mL) were added 1,3-difluoro-5-iodobenzene (610 mg, 2.5 mmol), CuI (120 mg, 0.63 mmol), trans-N¹,N²-dimethylcyclohexane-1,2-diamine (180 mg, 1.3 mmol) and K₃PO₄ (800 mg, 3.8 mmol). The resulting mixture was heated to 90° C. overnight. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0-15%, ethyl acetate in petroleum ether) to afford tert-butyl (S)-4-(5-bromo-7-(3,5-difluorophenyl)-7H-pyrrolo[2,3-d]pyrimidin-4-yl)-3-methylpiperazine-1-carboxylate (400 mg, 62%) as a white solid. LC/MS ESI (m/z): 508, 510 (M+H)⁺.

### Step 5. tert-Butyl (S)-4-(7-(3,5-difluorophenyl)-5-(2-oxoazetidin-1-yl)-7H-pyrrolo[2,3-d]pyrimidin-4-yl)-3-methylpiperazine-1-carboxylate

To a solution of tert-butyl (S)-4-(5-bromo-7-(3,5-difluorophenyl)-7H-pyrrolo[2,3-d]pyrimidin-4-yl)-3-methylpiperazine-1-carboxylate (400 mg, 0.79 mmol) in DMF (10 mL) were added azetidin-2-one (110 mg, 1.6 mmol), CuI (75 mg, 0.40 mmol), trans-N¹,N²-dimethylcyclohexane-1,2-diamine (110 mg, 0.79 mmol) and K₃PO₄ (500 mg, 2.4 mmol). The resulting mixture was heated to 90° C. overnight. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0-15%, ethyl acetate in petroleum ether) to afford tert-butyl (S)-4-(7-(3,5-difluorophenyl)-5-(2-oxoazetidin-1-yl)-7H-pyrrolo[2,3-d]pyrimidin-4-yl)-3-methylpiperazine-1-carboxylate (280 mg, 72%) as a white solid. LC/MS ESI (m/z): 499 (M+H)⁺.

### Step 6. tert-Butyl (S)-4-(5-(azetidin-1-yl)-7-(3,5-difluorophenyl)-7H-pyrrolo[2,3-d]pyrimidin-4-yl)-3-methylpiperazine-1-carboxylate

To a solution of tert-butyl (S)-4-(7-(3,5-difluorophenyl)-5-(2-oxoazetidin-1-yl)-7H-pyrrolo[2,3-d]pyrimidin-4-yl)-3-methylpiperazine-1-carboxylate (280 mg, 0.56 mmol) in THE (5 mL) were added RhH(CO)(PPh)₃ (260 mg, 0.28 mmol) and PhSiH₃ (0.34 ml, 2.8 mmol) at 0° C. The resulting mixture was stirred at room temperature for 1 h. The reaction was quenched with water and extracted twice with EtOAc. The combined organic layers were washed with brine, dried over Na₂SO₄, filtered and concentrated. The residue was purified by flash column chromatography (silica gel, ethyl acetate in petroleum ether) to give crude product which was further purified by prep-HPLC to afford tert-butyl (S)-4-(5-(azetidin-1-yl)-7-(3,5-difluorophenyl)-7H-pyrrolo[2,3-d]pyrimidin-4-yl)-3-methylpiperazine-1-carboxylate (110 mg, 39%) as a white solid. LC/MS ESI (m/z): 485 (M+H)⁺. ¹H NMR (400 MHz, CDCl₃) δ 8.38 (s, 1H), 7.42 (d, J=7.5 Hz, 2H), 6.75-6.69 (m, 1H), 6.59 (s, 1H), 5.00-4.90 (m, 1H), 4.20-3.89 (m, 3H), 3.81-3.75 (m, 2H), 3.66-3.60 (m, 2H), 3.55-3.38 (m, 2H), 3.05-2.90 (m, 1H), 2.32-2.25 (m, 2H), 1.49 (s, 9H), 1.16 (d, J=6.5 Hz, 3H).

### Example 152. Synthesis of tert-butyl (2S,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate (Compound 501)

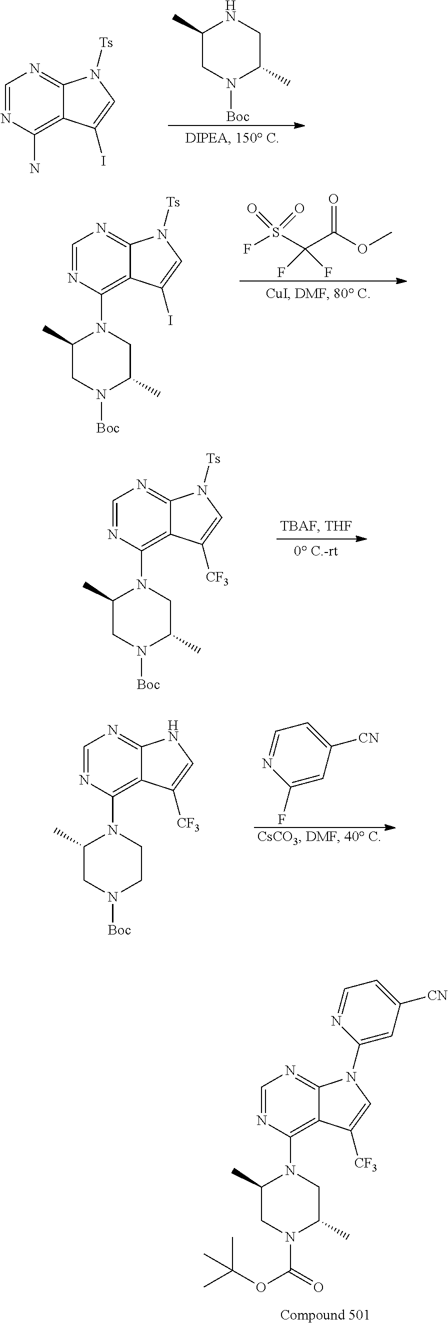

### Step 1. tert-Butyl (2S,5R)-4-(5-iodo-7-tosyl-7H-pyrrolo[2,3-d]pyrimidin-4-yl)-2,5-dimethylpiperazine-1-carboxylate

To the solution of 4-chloro-5-iodo-7-tosyl-7H-pyrrolo[2,3-d]pyrimidine (1.0 g, 2.3 mmol, prepared following the procedure outlined in Compound 536 in DIPEA (5 mL) was added tert-butyl (2S,5R)-2,5-dimethylpiperazine-1-carboxylate (1.0 mL, 4.6 mmol). The resulting reaction mixture was stirred at 150° C. under N₂ for 40 min. After cooling to room temperature, solvent was removed and the residue was purified by flash column chromatography (silica gel, 0-30%, ethyl acetate in petroleum ether) to afford to afford tert-butyl (2S,5R)-4-(5-iodo-7-tosyl-7H-pyrrolo[2,3-d]pyrimidin-4-yl)-2,5-dimethylpiperazine-1-carboxylate (1.3 g, 92%) as a white solid. LC/MS ESI (m/z): 612 (M+H)⁺.

### Step 2. tert-Butyl (2S,5R)-2,5-dimethyl-4-(7-tosyl-5-(trifluoromethyl)-7H-pyrrolo[2,3-d]pyrimidin-4-yl)piperazine-1-carboxylate

To the solution of tert-butyl (2S,5R)-4-(5-iodo-7-tosyl-7H-pyrrolo[2,3-d]pyrimidin-4-yl)-2,5-dimethylpiperazine-1-carboxylate (500 mg, 0.82 mmol) in DMF (5 mL) were added CuI (630 mg, 0.82 mmol) and methyl 2,2-difluoro-2-(fluorosulfonyl)acetate (0.41 mL, 3.3 mmol) respectively. The resulting reaction mixture was stirred at 80° C. under N₂ overnight. After cooling to room temperature, the reaction was partitioned between EtOAc and water. The aqueous layer was extracted twice with EtOAc. The combined organic layers were washed with brine, dried over Na₂SO₄, filtered, and concentrated under reduced pressure. The residue was purified by flash column chromatography (silica gel, 0-20%, ethyl acetate in petroleum ether) to obtain tert-butyl (2S,5R)-2,5-dimethyl-4-(7-tosyl-5-(trifluoromethyl)-7H-pyrrolo[2,3-d]pyrimidin-4-yl)piperazine-1-carboxylate (250 mg, 55%) as a white solid. LC/MS ESI (m/z): 554 (M+H)⁺.

### Step 3. tert-Butyl (2S,5R)-2,5-dimethyl-4-(5-(trifluoromethyl)-7H-pyrrolo[2,3-d]pyrimidin-4-yl)piperazine-1-carboxylate

To a solution of tert-butyl (2S,5R)-2,5-dimethyl-4-(7-tosyl-5-(trifluoromethyl)-7H-pyrrolo[2,3-d]pyrimidin-4-yl)piperazine-1-carboxylate (250 mg, 0.45 mmol) in THE (7 mL) at 0° C. was added TBAF (2.0 mL, 1.0 M in THF). After stirring at room temperature under N₂ for 1.5 h, the reaction mixture was quenched with ice water and extracted twice with EtOAc. The combined organic layers were washed with aqueous saturated NH₄Cl solution, dried over Na₂SO₄, filtered, and concentrated under reduced pressure. The residue was purified by flash column chromatography (silica gel, 0˜50%, ethyl acetate in petroleum ether) to obtain tert-butyl (2S,5R)-2,5-dimethyl-4-(5-(trifluoromethyl)-7H-pyrrolo[2,3-d]pyrimidin-4-yl)piperazine-1-carboxylate (170 mg, 91%) as a white solid. LC/MS ESI (m/z): 400 (M+H)⁺.

### Step 4. tert-Butyl (2S,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate

To a solution of tert-butyl (2S,5R)-2,5-dimethyl-4-(5-(trifluoromethyl)-7H-pyrrolo[2,3-d]pyrimidin-4-yl)piperazine-1-carboxylate (170 mg, 0.41 mmol) in DMF (3 mL) were added Cs₂CO₃ (270 mg, 0.83 mmol) and 2-fluoroisonicotinonitrile (100 mg, 0.83 mmol) respectively. The resulting mixture was stirred at 40° C. under N2 overnight. After cooling to room temperature, the reaction was partitioned between EtOAc and water. The aqueous layer was extracted twice with EtOAc. The combined organic layers were washed with brine, dried over Na₂SO₄, filtered, and concentrated under reduced pressure. The residue was purified by prep-HPLC to obtain tert-butyl (2S,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate (130 mg, 60%) as a white solid. LC/MS(ESI) m/z: 502 (M+H)⁺. ¹H NMR (400 MHz, CDCl₃) δ 9.28-9.27 (m, 1H), 8.61-8.59 (m, 1H), 8.57 (s, 1H), 8.50 (s, 1H), 7.41 (dd, J=5.0, 1.3 Hz, 1H), 4.55-4.48 (m, 1H), 4.39-4.23 (m, 1H), 3.74-3.69 (m, 1H), 3.69-3.64 (m, 1H), 3.59-3.54 (m, 1H), 3.43-3.39 (m, 1H), 1.42 (s, 9H), 1.09 (d, J=6.6 Hz, 3H), 1.03 (d, J=6.8 Hz, 3H).

The following compounds were prepared by procedures similar to the synthesis of compound 501 from the corresponding amines.

| Cmpd | | |
|---|---|---|
| No. | Chemical Name | LCMS and ¹H NMR |
| 502 | tert-butyl (25,5S)-4-(7-(4- | LC/MS ESI (m/z): 502 (M + H)⁺. ¹H NMR (400 MHz, |
| | cyanopyridin-2-yl)-5- | CDCl₃) δ 9.35 (s, 1H), 8.68-8.62 (m, 3H), 7.50-7.47 (m, |
| | (trifluoromethyl)-7H- | 1H), 4.50-4.41 (m, 1H), 4.11-3.97 (m, 2H), 3.68-3.61 |
| | pyrrolo[2,3-d]pyrimidin-4-yl)- | (m, 1H), 3.37-3.31 (m, 1H), 3.10-3.03 (m, 1H), 1.47 (s, |
| | 2,5-dimethylpiperazine-1- | 9H), 1.21 (d, J = 6.3 Hz, 3H), 1.10 (d, J = 6.2 Hz, 3H). |
| | carboxylate | |
| 503 | tert-butyl (2R,5R)-4-(7-(4- | LC/MS ESI (m/z): 502 (M + H)⁺. ¹H NMR (400 MHz, |
| | cyanopyridin-2-yl)-5- | CDCl₃) δ 9.35 (s, 1H), 8.68-8.65 (m, 2H), 8.63 (s, 1H), |
| | (trifluoromethyl)-7H- | 7.48 (dd, J = 5.0, 1.3 Hz, 1H), 4.50-4.41 (m, 1H), 4.12- |
| | pyrrolo[2,3-d]pyrimidin-4-yl)- | 3.97 (m, 2H), 3.65 (dd, J = 13.2, 5.4 Hz, 1H), 3.34 (dd, J = |
| | 2,5-dimethylpiperazine-1- | 13.2, 8.2 Hz, 1H), 3.06 (dd, J = 13.9, 11.5 Hz, 1H), 1.49- |
| | carboxylate | 1.46 (m, 9H), 1.21 (d, J = 6.3 Hz, 3H), 1.10 (d, J = 6.2 Hz, |
| | | 3H). |

### Example 153. Synthesis of tert-butyl (2R,5S)-4-(7-(4-cyanopyridin-2-yl)-5-(2-oxa-6-azaspiro[3.3]heptan-6-yl)-7H-pyrrolo[2,3-d]pyrimidin-4-yl)-2,5-dimethylpiperazine-1-carboxylate (Compound 504)

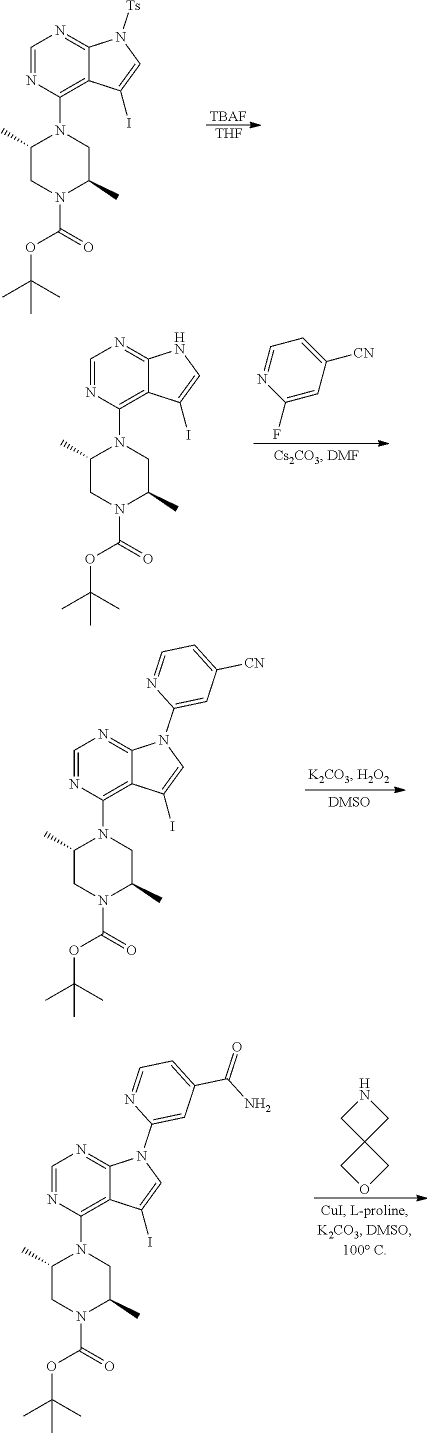

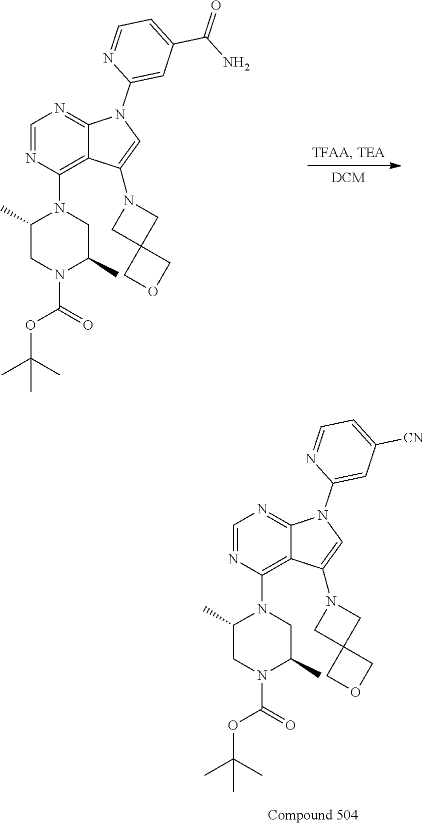 [SMILES: C[C@@H]1CN(c2ncnc3c2c(N2CC4(COC4)C2)cn3-c2cc(C(N)=O)ccn2)[C@@H](C)CN1C(=O)OC(C)(C)C ; C[C@@H]1CN(c2ncnc3c2c(N2CC4(COC4)C2)cn3-c2cc(C#N)ccn2)[C@@H](C)CN1C(=O)OC(C)(C)C]

### Step 1. tert-Butyl (2R,5S)-4-(5-iodo-7H-pyrrolo[2,3-d]pyrimidin-4-yl)-2,5-dimethylpiperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (10 g, 16 mmol, prepared following the procedure outlined in Compound 536 in THE (50 mL) was added TBAF (19 mL, 1.0 M in THF). After stirring at room temperature for 2 h, the reaction mixture was quenched with water and extracted twice with EtOAc. The combined organic layers were washed with brine, dried over Na₂SO₄, filtered and concentrated. The residue was purified by flash column chromatography (silica gel, 0-40%, ethyl acetate in petroleum ether) to afford tert-butyl (2R,5S)-4-(5-iodo-7H-pyrrolo[2,3-d]pyrimidin-4-yl)-2,5-dimethylpiperazine-1-carboxylate (6.0 g, 80%) as a yellow solid. LC/MS ESI (m/z): 458 (M+H)⁺.

### Step 2. tert-Butyl (2R,5S)-4-(7-(4-cyanopyridin-2-yl)-5-iodo-7H-pyrrolo[2,3-d]pyrimidin-4-yl)-2,5-dimethylpiperazine-1-carboxylate

To a solution of tert-butyl (2R,5S)-4-(5-iodo-7H-pyrrolo[2,3-d]pyrimidin-4-yl)-2,5-dimethylpiperazine-1-carboxylate (6.0 g, 15 mmol) in DMF (100 mL) were added 2-fluoroisonicotinonitrile (3.2 g, 31 mmol) and Cs₂CO₃ (8.6 g, 31 mmol). The resulting mixture was heated to 40° C. for 2 h. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to afford tert-butyl (2R,5S)-4-(7-(4-cyanopyridin-2-yl)-5-iodo-7H-pyrrolo[2,3-d]pyrimidin-4-yl)-2,5-dimethylpiperazine-1-carboxylate (2.7 g, 35%) as a yellow solid. LC/MS ESI (m/z): 560 (M+H)⁺.

### Step 3. tert-Butyl (2R,5S)-4-(7-(4-carbamoylpyridin-2-yl)-5-iodo-7H-pyrrolo[2,3-d]pyrimidin-4-yl)-2,5-dimethylpiperazine-1-carboxylate

To a solution of tert-butyl (2R,5S)-4-(7-(4-cyanopyridin-2-yl)-5-iodo-7H-pyrrolo[2,3-d]pyrimidin-4-yl)-2,5-dimethylpiperazine-1-carboxylate (1.0 g, 1.8 mmol) and K₂CO₃ (0.99 g, 7.2 mmol) in DMSO (10 mL) at 0° C. was added H₂O₂ (1.0 mL). After stirring at room temperature for 1.5 h under N₂, the reaction was quenched with Na₂S₂O₃ (aq.) and poured into water. The resultant solids were collected by filtration and dried in vacuo to afford tert-butyl (2R,5S)-4-(7-(4-carbamoylpyridin-2-yl)-5-iodo-7H-pyrrolo[2,3-d]pyrimidin-4-yl)-2,5-dimethylpiperazine-1-carboxylate (0.88 g, 85%) as a white solid. LC/MS ESI (m/z): 578 (M+H)⁺.

### Step 4. tert-Butyl (2R,5S)-4-(7-(4-carbamoylpyridin-2-yl)-5-(2-oxa-6-azaspiro[3.3]heptan-6-yl)-7H-pyrrolo[2,3-d]pyrimidin-4-yl)-2,5-dimethylpiperazine-1-carboxylate

To a solution of tert-butyl (2R,5S)-4-(7-(4-carbamoylpyridin-2-yl)-5-iodo-7H-pyrrolo[2,3-d]pyrimidin-4-yl)-2,5-dimethylpiperazine-1-carboxylate (400 mg, 0.69 mmol) in DMSO (10 mL) were added CuI (66 mg, 0.34 mmol), L-proline (80 mg, 0.69 mmol), K₂CO₃ (570 mg, 4.2 mmol) and 2-oxa-6-azaspiro[3.3]heptane (270 mg, 2.8 mmol). The resulting mixture was stirred at 100° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0-20%, methanol in dichloromethane) to afford crude tert-butyl (2R,5S)-4-(7-(4-carbamoylpyridin-2-yl)-5-(2-oxa-6-azaspiro[3.3]heptan-6-yl)-7H-pyrrolo[2,3-d]pyrimidin-4-yl)-2,5-dimethylpiperazine-1-carboxylate (200 mg, 52%) as a brown oil. LC/MS ESI (m/z): 549 (M+H)⁺.

### Step 5. tert-Butyl (2R,5S)-4-(7-(4-cyanopyridin-2-yl)-5-(2-oxa-6-azaspiro[3.3]heptan-6-yl)-7H-pyrrolo[2,3-d]pyrimidin-4-yl)-2,5-dimethylpiperazine-1-carboxylate

To a solution of tert-butyl (2R,5S)-4-(7-(4-carbamoylpyridin-2-yl)-5-(2-oxa-6-azaspiro[3.3]heptan-6-yl)-7H-pyrrolo[2,3-d]pyrimidin-4-yl)-2,5-dimethylpiperazine-1-carboxylate (60 mg, 0.11 mmol) and TEA (1.0 mL, 81 mmol) in DCM (5 mL) at 0° C. was added TFAA (0.30 mL, 0.27 mmol) dropwise. After stirring at 0° C. for 20 min, the reaction was quenched with water and extracted twice with DCM. The combined organic layers were washed with brine, dried over Na₂SO₄, filtered and concentrated. The residue was purified by flash column chromatography (silica gel, 0-30%, ethyl acetate in petroleum ether) to give crude product which was further purified by prep-HPLC to afford tert-butyl (2R,5S)-4-(7-(4-cyanopyridin-2-yl)-5-(2-oxa-6-azaspiro[3.3]heptan-6-yl)-7H-pyrrolo[2,3-d]pyrimidin-4-yl)-2,5-dimethylpiperazine-1-carboxylate (16 mg, 28%) as a yellow solid. LC/MS ESI (m/z): 531 (M+H)⁺. ¹H NMR (400 MHz, CDCl₃) δ 9.27 (s, 1H), 8.50 (d, J=5.0 Hz, 1H), 8.36 (s, 1H), 7.38 (s, 1H), 7.23 (dd, J=5.0, 1.3 Hz, 1H), 5.01-4.93 (m, 1H), 4.77 (s, 4H), 4.39-4.14 (m, 1H), 3.93 (d, J=7.5 Hz, 2H), 3.91-3.84 (m, 1H), 3.71 (d, J=7.4 Hz, 2H), 3.69-3.48 (m, 3H), 1.42 (s, 9H), 1.12 (d, J=6.4 Hz, 3H), 0.91 (d, J=6.4 Hz, 3H).

### Example 154. Synthesis of tert-butyl (2R,5S)-4-(7-(4-chloropyridin-2-yl)-5-(trifluoromethyl)-7H-pyrrolo[2,3-d]pyrimidin-4-yl)-2,5-dimethylpiperazine-1-carboxylate (Compound 506)

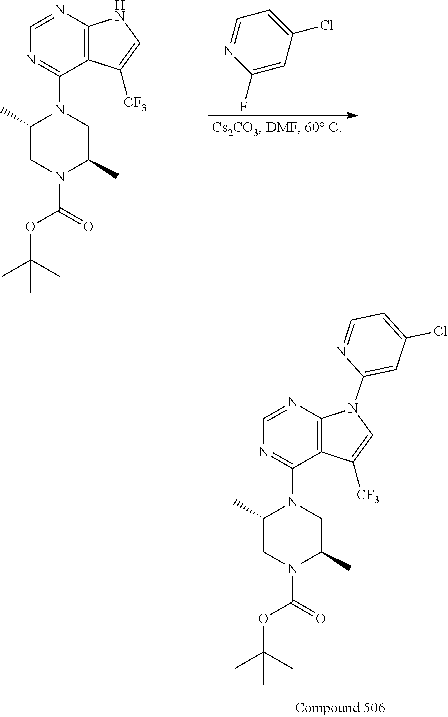 [SMILES: C[C@@H]1CN(c2ncnc3[nH]cc(C(F)(F)F)c23)[C@@H](C)CN1C(=O)OC(C)(C)C ; Fc1cc(Cl)ccn1 ; C[C@@H]1CN(c2ncnc3c2c(C(F)(F)F)cn3-c2cc(Cl)ccn2)[C@@H](C)CN1C(=O)OC(C)(C)C]

To a solution of tert-butyl (2R,5S)-2,5-dimethyl-4-(5-(trifluoromethyl)-7H-pyrrolo[2,3-d]pyrimidin-4-yl)piperazine-1-carboxylate (300 mg, 0.75 mmol, prepared following the procedure outlined in Compound 536 in DMF (5 mL) were added 4-chloro-2-fluoropyridine (200 mg, 1.5 mmol) and Cs₂CO₃ (2.5 g, 7.5 mmol). After stirring at 60° C. under N₂ atmosphere overnight, the reaction was cooled to room temperature and partitioned between EtOAc and water. The aqueous layer was extracted twice with EtOAc. The combined organic layers were washed with brine, dried and concentrated. The residue was purified by flash column chromatography to give crude product, which was further purified by prep-HPLC to afford tert-butyl (2R,5S)-4-(7-(4-chloropyridin-2-yl)-5-(trifluoromethyl)-7H-pyrrolo[2,3-d]pyrimidin-4-yl)-2,5-dimethylpiperazine-1-carboxylate (280 mg, 73%) as a white solid. LC/MS ESI (m/z): 511 (M+H)⁺. ¹H NMR (400 MHz, CDCl₃) δ 8.92 (d, J=1.7 Hz, 1H), 8.57-8.54 (m, 1H), 8.50 (s, 1H), 8.33 (d, J=5.3 Hz, 1H), 7.21 (dd, J=5.3, 1.8 Hz, 1H), 4.53-4.45 (m, 1H), 4.40-4.19 (m, 1H), 3.73-3.64 (m, 2H), 3.59-3.54 (m, 1H), 3.42-3.37 (m, 1H), 1.42 (s, 9H), 1.08-1.03 (m, 6H).

### Example 155. Synthesis of tert-butyl (2R,5S)-4-(7-(4-cyanopyridin-2-yl)-5-propoxy-7H-pyrrolo[2,3-d]pyrimidin-4-yl)-2,5-dimethylpiperazine-1-carboxylate (Compound 507)

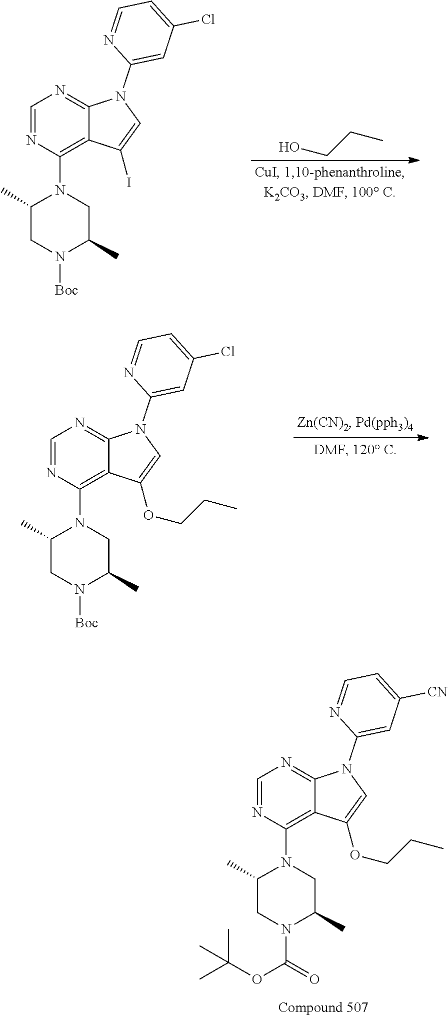

### Step 1. tert-Butyl (2R,5S)-4-(7-(4-chloropyridin-2-yl)-5-propoxy-7H-pyrrolo[2,3-d]pyrimidin-4-yl)-2,5-dimethylpiperazine-1-carboxylate

To a solution of tert-butyl (2R,5S)-4-(7-(4-chloropyridin-2-yl)-5-iodo-7H-pyrrolo[2,3-d]pyrimidin-4-yl)-2,5-dimethylpiperazine-1-carboxylate (20 mg, 0.036 mmol, prepared following the procedure outlined in Compound 521 in propan-1-ol (0.5 mL) were added CuI (7.0 mg, 0.021 mmol), 1,10-phenanthroline (0.007 mL, 0.044 mmol) and K₂CO₃ (12 mg, 0.087 mmol) respectively. The resulting reaction mixture was stirred at 100° C. under N₂ overnight. The reaction was repeated at the same scale four additional times. After cooling to room temperature, the reaction mixtures from the five reactions were combined, solvent was removed and the residue was purified by column chromatography on silica gel (0-20% ethyl acetate in petroleum ether) to afford tert-butyl (2R,5S)-4-(7-(4-chloropyridin-2-yl)-5-propoxy-7H-pyrrolo[2,3-d]pyrimidin-4-yl)-2,5-dimethylpiperazine-1-carboxylate (7.0 mg, 8.0%) as a white solid. LC/MS(ESI) (m/z): 501 (M+H)⁺.

### Step 2. tert-Butyl (2R,5S)-4-(7-(4-cyanopyridin-2-yl)-5-propoxy-7H-pyrrolo[2,3-d]pyrimidin-4-yl)-2,5-dimethylpiperazine-1-carboxylate

To a solution of tert-butyl (2R,5S)-4-(7-(4-chloropyridin-2-yl)-5-propoxy-7H-pyrrolo[2,3-d]pyrimidin-4-yl)-2,5-dimethylpiperazine-1-carboxylate (7.0 mg, 0.014 mmol) in DMF (2 mL) were added Pd(PPh₃)₄ (28 mg, 0.024 mmol) and Zn(CN)₂ (4.0 mg, 0.034 mmol) respectively. After stirring at 120° C. under N2 overnight, the reaction mixture was filtered and the filtrate was concentrated under reduced pressure. The residue was purified by prep-HPLC to afford tert-butyl (2R,5S)-4-(7-(4-cyanopyridin-2-yl)-5-propoxy-7H-pyrrolo[2,3-d]pyrimidin-4-yl)-2,5-dimethylpiperazine-1-carboxylate (4.0 mg, 57%) as a white solid. LC/MS(ESI) (m/z): 492 (M+H)⁺. ¹H NMR (400 MHz, CDCl₃) δ 9.38 (s, 1H), 8.57-8.54 (m, 1H), 8.39 (s, 1H), 7.54 (s, 1H), 7.30-7.29 (m, 0.4H, rotamer), 7.28-7.28 (m, 0.6H, rotamer), 5.08-5.01 (m, 1H), 4.43-4.22 (m, 2H), 4.06-3.99 (m, 2H), 3.85-3.79 (m, 0.5H, rotamer), 3.75-3.69 (m, 0.5H, rotamer), 3.60-3.54 (m, 1H), 3.48-3.38 (m, 1H), 1.91 (dd, J=14.0, 6.8 Hz, 2H), 1.50 (s, 9H), 1.26-1.25 (m, 3H), 1.18 (d, J=6.7 Hz, 3H), 1.08 (t, J=7.4 Hz, 3H).

The following compound was prepared by a procedure similar to the synthesis of compound 507 from the corresponding alcohol.

| Cmpd | | |
|---|---|---|
| No. | Chemical Name | LCMS and ¹H NMR |
| 508 | tert-butyl (2R,5S)-4-(7-(4- | LC/MS ESI (m/z): 504 (M + H)⁺. ¹H NMR (400 MHz, |
| | cyanopyridin-2-yl)-5- | CDCl₃) δ 9.36 (s, 1H), 8.55 (d, J = 5.0 Hz, 1H), 8.38 (s, |
| | cyclobutoxy-7H-pyrrolo[2,3- | 1H), 7.42 (s, 1H), 7.30-7.28 (m, 1H), 4.99 (s, 1H), 4.70- |
| | d]pyrimidin-4-yl)-2,5- | 4.63 (m, 1H), 4.56-4.47 (m, 0.5H, rotamer), 4.42-4.27 |
| | dimethylpiperazine-1- | (m, 1.5H, rotamer), 3.86-3.71 (m, 1H), 3.61-3.55 (m, |
| | carboxylate | 1H), 3.48-3.38 (m, 1H), 2.59-2.51 (m, 2H), 2.26-2.20 |
| | | (m, 2H), 1.96-1.88 (m, 1H), 1.78-1.71 (m, 1H), 1.51 (s, |
| | | 9H), 1.27-1.25 (m, 3H), 1.22-1.18 (m, 3H). |

### Example 156. Synthesis of tert-butyl (2R,5S)-4-(7-(6-cyanopyrimidin-4-yl)-5-(trifluoromethyl)-7H-pyrrolo[2,3-d]pyrimidin-4-yl)-2,5-dimethylpiperazine-1-carboxylate (Compound 509)

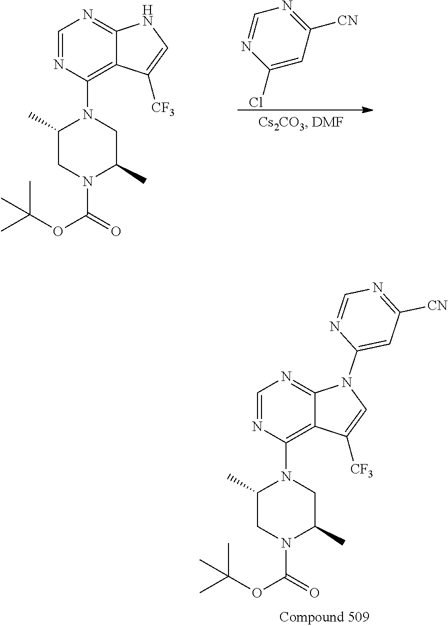 [SMILES: C[C@@H]1CN(c2ncnc3[nH]cc(C(F)(F)F)c23)[C@@H](C)CN1C(=O)OC(C)(C)C ; N#Cc1cc(Cl)ncn1 ; C[C@@H]1CN(c2ncnc3c2c(C(F)(F)F)cn3-c2cc(C#N)ncn2)[C@@H](C)CN1C(=O)OC(C)(C)C]

A mixture of tert-butyl (2R,5S)-2,5-dimethyl-4-(5-(trifluoromethyl)-7H-pyrrolo[2,3-d]pyrimidin-4-yl)piperazine-1-carboxylate (200 mg, 0.50 mmol, prepared following the procedure outlined in Compound 536, 6-chloropyrimidine-4-carbonitrile (140 mg, 1.0 mmol) and Cs₂CO₃ (330 mg, 1.0 mmol) in DMF (10 mL) was stirred at room temperature for 2 h. The reaction mixture was partitioned between EtOAc and water. The aqueous layer was extracted twice with EtOAc. The combined organic phases were washed with brine, dried over Na₂SO₄, filtered and concentrated in vacuo. The residue was purified by flash column chromatography (silica gel, 0˜30%, ethyl acetate in petroleum ether) to afford tert-butyl (2R,5S)-4-(7-(6-cyanopyrimidin-4-yl)-5-(trifluoromethyl)-7H-pyrrolo[2,3-d]pyrimidin-4-yl)-2,5-dimethylpiperazine-1-carboxylate (130 mg, 51%) as a white solid. LC/MS ESI (m/z): 503 (M+H)⁺. ¹H NMR (400 MHz, CDCl₃) δ 9.60 (d, J=1.2 Hz, 1H), 9.18 (d, J=1.2 Hz, 1H), 8.68 (d, J=0.8 Hz, 1H), 8.59 (s, 1H), 4.61 (br. s, 1H), 4.38 (br. s, 1H), 3.83-3.71 (m, 2H), 3.65-3.59 (m, 1H), 3.48 (d, J=13.4 Hz, 1H), 1.49 (s, 9H), 1.18 (d, J=6.6 Hz, 3H), 1.08 (d, J=6.8 Hz, 3H).

### Example 157. Synthesis of tert-butyl (2R,5S)-4-(7-(4-cyanopyrimidin-2-yl)-5-(trifluoromethyl)-7H-pyrrolo[2,3-d]pyrimidin-4-yl)-2,5-dimethylpiperazine-1-carboxylate (Compound 510)

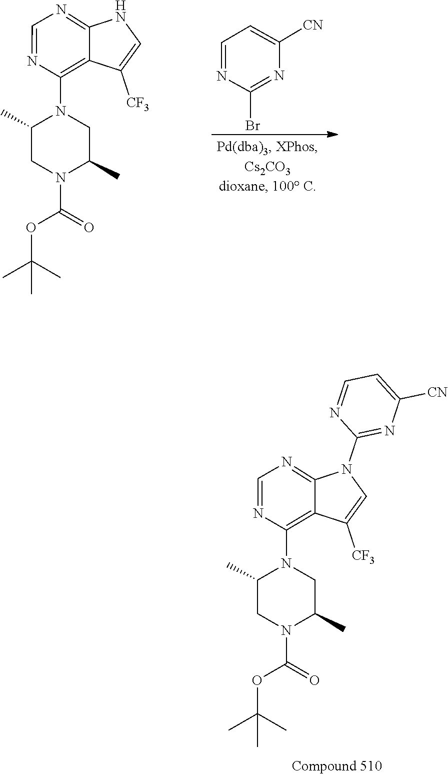 [SMILES: C[C@@H]1CN(c2ncnc3[nH]cc(C(F)(F)F)c23)[C@@H](C)CN1C(=O)OC(C)(C)C ; N#Cc1ccnc(Br)n1 ; C[C@@H]1CN(c2ncnc3c2c(C(F)(F)F)cn3-c2nccc(C#N)n2)[C@@H](C)CN1C(=O)OC(C)(C)C]

To a solution of tert-butyl (2R,5S)-2,5-dimethyl-4-(5-(trifluoromethyl)-7H-pyrrolo[2,3-d]pyrimidin-4-yl)piperazine-1-carboxylate (300 mg, 0.75 mmol, prepared following the procedure outlined in Compound 536 in dioxane (10 mL) were added 2-bromopyrimidine-4-carbonitrile (280 mg, 1.5 mmol), Pd₂(dba)₃ (230 mg, 0.25 mmol), X-Phos (290 mg, 0.50 mmol) and Cs₂CO₃ (490 mg, 1.5 mmol). The resulting mixture was heated to 100° C. overnight under N₂. After cooling to room temperature, solvent was removed and the residue was purified by flash column chromatography (silica gel, 0-10%, MeOH in DCM) and prep-HPLC to afford tert-butyl (2R,5S)-4-(7-(4-cyanopyrimidin-2-yl)-5-(trifluoromethyl)-7H-pyrrolo[2,3-d]pyrimidin-4-yl)-2,5-dimethylpiperazine-1-carboxylate (180 mg, 46%). LC/MS ESI (m/z): 503 (M+H)⁺. ¹H NMR (400 MHz, CD₃OD) δ 9.18 (d, J=4.9 Hz, 1H), 8.66-8.63 (m, 1H), 8.56 (s, 1H), 7.93 (d, J=4.9 Hz, 1H), 4.66-4.58 (m, 1H), 4.39-4.31 (m, 1H), 3.84-3.73 (m, 2H), 3.68-3.61 (m, 1H), 3.52 (d, J=12.8 Hz, 1H), 1.50 (s, 9H), 1.16 (d, J=6.6 Hz, 3H), 1.10 (d, J=6.8 Hz, 3H).

### Example 158. Synthesis of pentan-3-yl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (Compound 512)

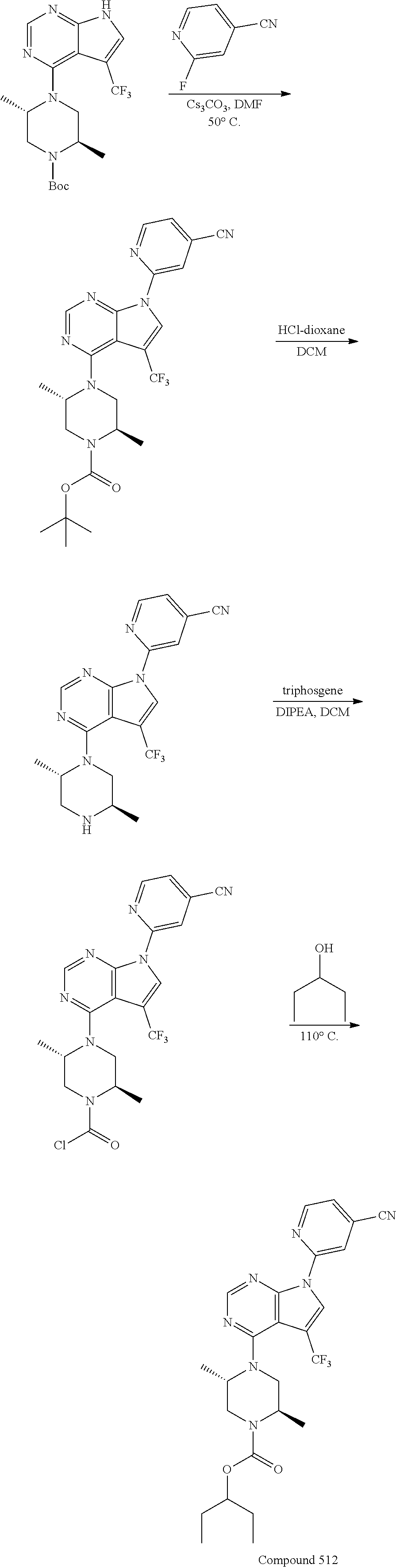

### Step 11. tert-Butyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate

To a solution of tert-butyl (2R,5S)-2,5-dimethyl-4-(5-(trifluoromethyl)-7H-pyrrolo[2,3-d]pyrimidin-4-yl)piperazine-1-carboxylate (6.0 g, 15 mmol, prepared following the procedure outlined in Compound 536 in DMF (70 mL) at 25° C. were added Cs₂CO₃ (9.8 g, 30 mmol) and 2-fluoroisonicotinonitrile (9.2 g, 75 mmol). The reaction was heated to 50° C. and stirred for 1 h. After cooling to room temperature, the resulting mixture was poured into H₂O (300 m1) and extracted twice with EtOAc (200 mL). The combined organic phases were washed twice with brine (200 mL), dried over Na₂SO₄, filtered, and concentrated in vacuo. The crude product was purified by silica gel column (0˜20 ethyl acetate in petroleum ether) to afford tert-butyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (6.0 g, 80%) as a white solid. LC/MS ESI (m/z): 502 (M+H)⁺. ¹H NMR (400 MHz, CDCl₃) 9.27 (d, J=0.9 Hz, 1H), 8.60 (d, J=5.0 Hz, 1H), 8.57 (s, 1H), 8.50 (s, 1H), 7.43-7.39 (m, 1H), 4.52 (s, 1H), 4.31 (s, 1H), 3.76-3.63 (m, 2H), 3.59-3.52 (m, 1H), 3.41 (d, J=13.3 Hz, 1H), 1.42 (s, 9H), 1.09 (d, J=6.6 Hz, 3H), 1.03 (d, J=6.8 Hz, 3H).

### Step 2. 2-(4-((2S,5R)-2,5-Dimethylpiperazin-1-yl)-5-(trifluoromethyl)-7H-pyrrolo[2,3-d]pyrimidin-7-yl)isonicotinonitrile

To a solution of tert-butyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (3.0 g, 6.0 mmol) in DCM (15 mL) was added HCl (10 mL, 4.0 M in dioxane). The resulting mixture was stirred at room temperature for 3 h. The reaction was treated with sat. NaHCO₃ (aq.) and extracted twice with DCM. The combined organic layers were dried over Na₂SO₄, filtered and concentrated to afford 2-(4-((2S,5R)-2,5-dimethylpiperazin-1-yl)-5-(trifluoromethyl)-7H-pyrrolo[2,3-d]pyrimidin-7-yl)isonicotinonitrile (2.0 g, 83%) as a white solid, which was used in the next step directly. LC/MS ESI (m/z): 402 (M+H)⁺.

### Step 3. (2R,5S)-4-(7-(4-Cyanopyridin-2-yl)-5-(trifluoromethyl)-7H-pyrrolo[2,3-d]pyrimidin-4-yl)-2,5-dimethylpiperazine-1-carbonyl chloride

To a solution of 2-(4-((2S,5R)-2,5-dimethylpiperazin-1-yl)-5-(trifluoromethyl)-7H-pyrrolo[2,3-d]pyrimidin-7-yl)isonicotinonitrile (300 mg, 0.75 mmol) in DCM (10 mL) were added DIPEA (190 mg, 1.5 mmol), followed by triphosgene (300 mg, 0.75 mmol) in portions. The resulting mixture was stirred at room temperature for 1 h. The reaction was quenched with ice water and extracted twice with DCM. The combined organic layers were washed with brine, dried over Na₂SO₄, filtered, and concentrated to afford (2R,5S)-4-(7-(4-cyanopyridin-2-yl)-5-(trifluoromethyl)-7H-pyrrolo[2,3-d]pyrimidin-4-yl)-2,5-dimethylpiperazine-1-carbonyl chloride, which was used in the next step directly. LC/MS ESI (m/z): 464.0 (M+H)⁺.

### Step 4. Pentan-3-yl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate

A mixture of (2R,5S)-4-(7-(4-cyanopyridin-2-yl)-5-(trifluoromethyl)-7H-pyrrolo[2,3-d]pyrimidin-4-yl)-2,5-dimethylpiperazine-1-carbonyl chloride (340 mg, 0.75 mmol) and pentan-3-ol (5 mL) was stirred at 110° C. for 3 h under N₂. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography to give crude product which was further purified by prep-HPLC to afford pentan-3-yl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (250 mg, 65%) as a white solid. LC/MS ESI (m/z): 516 (M+H)⁺. ¹H NMR (400 MHz, CDCl₃) δ 9.31-9.23 (m, 1H), 8.61-8.56 (m, 2H), 8.53-8.49 (m, 1H), 7.43-7.39 (m, 1H), 4.69-4.62 (m, 1H), 4.56-4.48 (m, 1H), 4.45-4.29 (m, 1H), 3.77-3.69 (m, 2H), 3.64-3.58 (m, 1H), 3.45-3.40 (m, 1H), 1.58-1.51 (m, 4H), 1.08 (dd, J=9.2, 6.9 Hz, 6H), 0.87-0.81 (m, 6H).

The following compound was prepared by a procedure similar to the synthesis of compound 512 from the corresponding alcohol.

| Cmpd | | |
|---|---|---|
| No. | Chemical Name | LCMS and ¹H NMR |
| 516 | cyclobutyl (2R,5S)-4-(7-(4- | LC/MS ESI (m/z): 500 (M + H)⁺. ¹H NMR (400 MHz, |
| | cyanopyridin-2-yl)-5- | CDCl₃) δ 9.27 (s, 1H), 8.61-8.57 (m, 2H), 8.51 (s, 1H), |
| | (trifluoromethyl)-7H- | 7.41 (dd, J = 5.0, 1.3 Hz, 1H), 4.96-4.88 (m, 1H), 4.56- |
| | pyrrolo[2,3-d]pyrimidin-4-yl)- | 4.47 (m, 1H), 4.44-4.27 (m, 1H), 3.75-3.68 (m, 2H), |
| | 2,5-dimethylpiperazine-1- | 3.64-3.56 (m, 1H), 3.44-3.38 (m, 1H), 2.33-2.26 (m, |
| | carboxylate | 2H), 2.08-1.96 (m, 2H), 1.75-1.67 (m, 1H), 1.59-1.53 |
| | | (m, 1H), 1.07 (dd, J = 13.2, 6.7 Hz, 6H). |

### Example 159. Synthesis of tert-butyl (S)-4-(7-(4-cyanopyridin-2-yl)-5-(difluoromethyl)-7H-pyrrolo[2,3-d]pyrimidin-4-yl)-3-methylpiperazine-1-carboxylate (Compound 514)

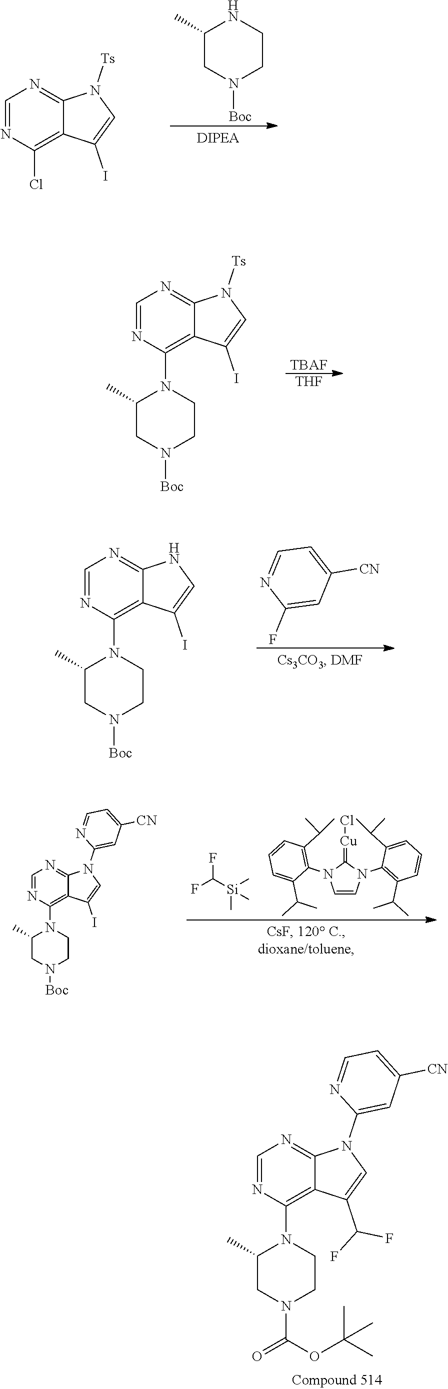

### Step 1. tert-Butyl (S)-4-(5-iodo-7-tosyl-7H-pyrrolo[2,3-d]pyrimidin-4-yl)-3-methylpiperazine-1-carboxylate

To a solution of 4-chloro-5-iodo-7-tosyl-7H-pyrrolo[2,3-d]pyrimidine (90 g, 0.21 mol) in DIPEA (600 mL) was added tert-butyl (S)-3-methylpiperazine-1-carboxylate (81 g, 0.42 mol). The resulting mixture was heated to 140° C. for 3 h. After cooling to room temperature, solvent was removed and the residue was purified by flash column chromatography (silica gel, 0˜30%, ethyl acetate in petroleum ether) to afford tert-butyl (S)-4-(5-iodo-7-tosyl-7H-pyrrolo[2,3-d]pyrimidin-4-yl)-3-methylpiperazine-1-carboxylate (100 g, 73%) as a white solid. LC/MS ESI (m/z): 598 (M+H)⁺.

### Step 2. tert-Butyl (S)-4-(5-iodo-7H-pyrrolo[2,3-d]pyrimidin-4-yl)-3-methylpiperazine-1-carboxylate

To a solution of tert-butyl (S)-4-(5-iodo-7-tosyl-7H-pyrrolo[2,3-d]pyrimidin-4-yl)-3-methylpiperazine-1-carboxylate (55 g, 110 mmol) in THE (400 mL) at 0° C. was added TBAF (440 mL, 1.0M in THF). After stirring at 0° C. for 1.5 h, the reaction was quenched with ice water and extracted twice with EtOAc. The combined organic layers were washed with brine, dried over Na₂SO₄, filtered and concentrated. The residue was purified by flash column chromatography (silica gel, 0˜10%, methanol in dichloromethane) to afford tert-butyl (S)-4-(5-iodo-7H-pyrrolo[2,3-d]pyrimidin-4-yl)-3-methylpiperazine-1-carboxylate (37 g, 91%) as a white solid. LC/MS ESI (m/z): 444 (M+H)⁺.

### Step 3. tert-Butyl (S)-4-(7-(4-cyanopyridin-2-yl)-5-iodo-7H-pyrrolo[2,3-d]pyrimidin-4-yl)-3-methylpiperazine-1-carboxylate

To a solution of tert-butyl 4-{5-cyclopropyl-7H-pyrrolo[2,3-d]pyrimidin-4-yl}piperidine-1-carboxylate (4.5 g, 10 mmol) and 2-fluoropyridine-4-carbonitrile (1.8 g, 15 mmol) in DMF (60 mL) was added Cs₂CO₃ (16 g, 50 mmol). The resulting mixture was stirred at 40° C. for 18 h. After cooling to room temperature, the reaction mixture was filtered. The filtrate was diluted with ice water and extracted twice with EtOAc. The combined organic layers were washed with brine, dried over Na₂SO₄, filtered and concentrated. The residue was purified by column chromatography on silica gel (0˜40% ethyl acetate in petroleum ether) to give tert-butyl (S)-4-(7-(4-cyanopyridin-2-yl)-5-iodo-7H-pyrrolo[2,3-d]pyrimidin-4-yl)-3-methylpiperazine-1-carboxylate (3.6 g, 6.6 mmol, 66%). LC/MS(ESI) m/z: 546 (M+H)⁺.

### Step 4. tert-Butyl (S)-4-(7-(4-cyanopyridin-2-yl)-5-(difluoromethyl)-7H-pyrrolo[2,3-d]pyrimidin-4-yl)-3-methylpiperazine-1-carboxylate

To a solution of tert-butyl (S)-4-(7-(4-cyanopyridin-2-yl)-5-iodo-7H-pyrrolo[2,3-d]pyrimidin-4-yl)-3-methylpiperazine-1-carboxylatetert-butyl 4-(5-cyclopropyl-7H-pyrrolo[2,3-d]pyrimidin-4-yl)piperazine-1-carboxylate (200 mg, 0.37 mmol) in dioxane/toluene (10 mL, 3:1 v/v) were added CsF (170 mg, 1.1 mmol), (difluoromethyl)trimethylsilane (920 mg, 7.5 mmol) and (1,3-bis(2,6-diisopropylphenyl)-1,3-dihydro-2H-imidazol-2-ylidene)copper(III) chloride (180 mg, 0.37 mmol). The resulting mixture was heated to 120° C. overnight. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to give crude product which was further purified by prep-HPLC to afford tert-butyl (S)-4-(7-(4-cyanopyridin-2-yl)-5-(difluoromethyl)-7H-pyrrolo[2,3-d]pyrimidin-4-yl)-3-methylpiperazine-1-carboxylate (15 mg, 8.6%) as a white solid. LC/MS ESI (m/z): 470 (M+H)⁺. ¹H NMR (400 MHz, CDCl₃) δ 9.34 (s, 1H), 8.67-8.53 (m, 3H), 7.48-7.42 (m, 1H), 7.00 (t, J=55.5 Hz, 1H), 4.38-4.29 (m, 1H), 3.97 (m, 1H), 3.75 (d, J=10.8 Hz, 1H), 3.63-3.47 (m, 2H), 3.44-3.38 (m, 1H), 3.22 (s, 1H), 1.50 (s, 9H), 1.23 (d, J=6.6 Hz, 3H).

The following compounds were prepared by procedures similar to the synthesis of compound 514 from the corresponding aryl halides.

| Cmpd | | |
|---|---|---|
| No. | Chemical Name | LCMS and ¹H NMR |
| 511 | tert-butyl (R)-4-(7-(4- | LC/MS ESI (m/z): 470 (M + H)⁺. ¹H NMR (400 MHz, |
| | cyanopyridin-2-yl)-5- | CDCl₃) δ 9.34 (s, 1H), 8.67-8.64 (m, 1H), 8.58 (s, 1H), |
| | (difluoromethyl)-7H- | 8.53 (t, J = 2.0 Hz, 1H), 7.48-7.44 (m, 1H), 6.93 (d, J = |
| | pyrrolo[2,3-d]pyrimidin-4-yl)-2- | 55.5 Hz, 1H), 4.41 (s, 1H), 4.08 (d, J = 12.8 Hz, 1H), 3.96 |
| | methylpiperazine-1-carboxylate | (d, J = 13.5 Hz, 1H), 3.88 (d, J = 12.9 Hz, 1H), 3.46-3.38 |
| | | (m, 2H), 3.15-3.08 (m, 1H), 1.50 (s, 9H), 1.21 (d, J = 6.8 |
| | | Hz, 3H). |
| 520 | tert-butyl (2R,5S)-4-(7-(4- | LC/MS ESI (m/z): 484 (M + H)⁺. ¹H NMR (400 MHz, |
| | cyanopyridin-2-yl)-5- | CDCl₃) δ 9.34 (s, 1H), 8.65 (d, J = 5.0 Hz, 1H), 8.56 (s, |
| | (difluoromethyl)-7H- | 1H), 8.51 (t, J = 1.9 Hz, 1H), 7.45 (dd, J = 5.0, 1.3 Hz, 1H), |
| | pyrrolo[2,3-d]pyrimidin-4-yl)- | 6.96 (t, J = 55.6 Hz, 1H), 4.62-4.53 (m, 1H), 4.52-4.28 |
| | 2,5-dimethylpiperazine-1- | (m, 1H), 3.84-3.72 (m, 2H), 3.61 (dd, J = 13.5, 3.6 Hz, |
| | carboxylate | 1H), 3.52 (d, J = 13.0 Hz, 1H), 1.49 (s, 9H), 1.18 (d, J = 6.6 |
| | | Hz, 3H), 1.13 (d, J = 6.8 Hz, 3H). |

### Example 160. Synthesis of cyclopropyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (Compound 515)

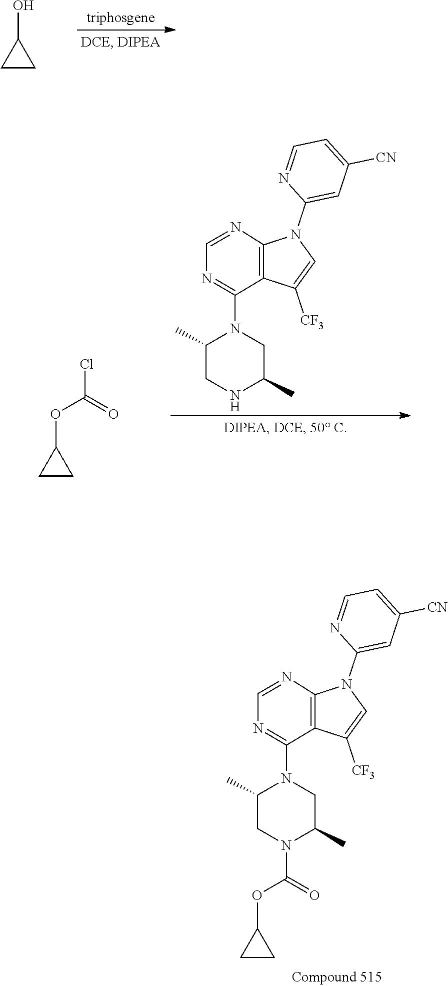

### Step 11. Cyclopropyl carbonochloridate

To a solution cyclopropanol (40 mg, 0.70 mmol) in DCE (8 mL) were added DIPEA (44 mg, 1.1 mmol) and triphosgene (68 mg, 0.24 mmol). The reaction was stirred at room temperature for 3 h and the resulting mixture was used in the next step directly.

### Step 2. Cyclopropyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate

To a solution of cyclopropyl carbonochloridate (0.70 mmol) in DCE (8 mL) were added DIPEA (44 mg, 1.1 mmol) and 2-(4-((2S,5R)-2,5-dimethylpiperazin-1-yl)-5-(trifluoromethyl)-7H-pyrrolo[2,3-d]pyrimidin-7-yl)isonicotinonitrile (200 mg, 0.50 mmol, prepared following the procedure outlined in Compound 512. The resulting mixture was stirred at 50° C. for 3 h under N₂. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography to give crude product which was further purified by prep-HPLC to afford cyclopropyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (170 mg, 66%) as a white solid. LC/MS ESI (m/z): 486 (M+H)⁺. ¹H NMR (400 MHz, CDCl₃) δ 9.27 (s, 1H), 8.61-8.57 (m, 2H), 8.51 (s, 1H), 7.42 (dd, J=5.0, 1.3 Hz, 1H), 4.65-4.27 (m, 2H), 4.09-4.04 (m, 1H), 3.73-3.57 (m, 3H), 3.44-3.38 (m, 1H), 1.10-1.03 (m, 6H), 0.67-0.61 (m, 4H).

The following compound was prepared by a procedure similar to the synthesis of Compound 515 from the corresponding aryl halogens.

| Cmpd | | |
|---|---|---|
| No. | Chemical Name | LCMS and ¹H NMR |
| 519 | 1-methylcyclopropyl (2R,5S)-4- | LC/MS ESI (m/z): 500 (M + H)⁺. ¹H NMR (400 MHz, |
| | (7-(4-cyanopyridin-2-yl)-5- | CDCl₃) δ 9.27 (s, 1H), 8.61-8.56 (m, 2H), 8.51 (s, 1H), |
| | (trifluoromethyl)-7H- | 7.41 (dd, J = 5.0, 1.2 Hz, 1H), 4.60-4.22 (m, 2H), 3.72- |
| | pyrrolo[2,3-d]pyrimidin-4-yl)- | 3.55 (m, 3H), 3.43-3.37 (m, 1H), 1.51 (s, 3H), 1.05 (dd, |
| | 2,5-dimethylpiperazine-1- | J = 14.1, 6.6 Hz, 6H), 0.85-0.80 (m, 2H), 0.62-0.56 (m, |
| | carboxylate | 2H). |

### Example 161. Synthesis of tert-butyl (2R,5S)-4-(7-(4-cyano-6-methylpyridin-2-yl)-5-(trifluoromethyl)-7H-pyrrolo[2,3-d]pyrimidin-4-yl)-2,5-dimethylpiperazine-1-carboxylate (Compound 517)

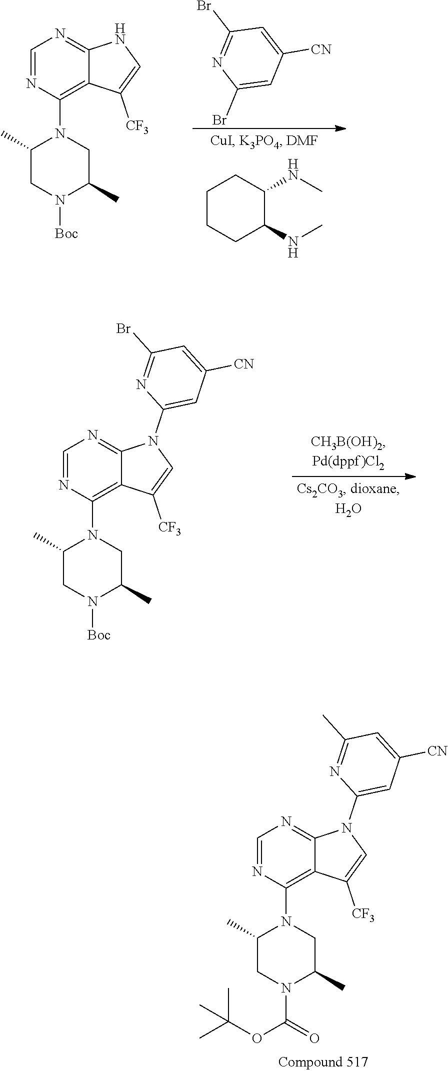

### Step 1. tert-Butyl (2R,5S)-4-(7-(6-bromo-4-cyanopyridin-2-yl)-5-(trifluoromethyl)-7H-pyrrolo[2,3-d]pyrimidin-4-yl)-2,5-dimethylpiperazine-1-carboxylate

To a solution of tert-butyl (2R,5S)-2,5-dimethyl-4-(5-(trifluoromethyl)-7H-pyrrolo[2,3-d]pyrimidin-4-yl)piperazine-1-carboxylate (300 mg, 0.75 mmol, prepared following the procedure outlined in Compound 536 in DMF (5 mL) were added 2,6-dibromoisonicotinonitrile (240 mg, 0.90 mmol), trans-N¹,N²-dimethylcyclohexane-1,2-diamine (28 mg, 0.20 mmol), CuI (140 mg, 0.75 mmol) and K₃PO₄ (320 mg, 1.5 mmol). The resulting mixture was heated to 90° C. for 1 h. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to give tert-butyl (2R,5S)-4-(7-(6-bromo-4-cyanopyridin-2-yl)-5-(trifluoromethyl)-7H-pyrrolo[2,3-d]pyrimidin-4-yl)-2,5-dimethylpiperazine-1-carboxylate (370 mg, 85%) as a yellow oil. LC/MS ESI (m/z): 580, 582 (M+H)⁺.

### Step 2. tert-Butyl (2R,5S)-4-(7-(4-cyano-6-methylpyridin-2-yl)-5-(trifluoromethyl)-7H-pyrrolo[2,3-d]pyrimidin-4-yl)-2,5-dimethylpiperazine-1-carboxylate

To a solution of tert-butyl (2R,5S)-4-(7-(6-bromo-4-cyanopyridin-2-yl)-5-(trifluoromethyl)-7H-pyrrolo[2,3-d]pyrimidin-4-yl)-2,5-dimethylpiperazine-1-carboxylate (160 mg, 0.30 mmol) in dioxane (5 mL) and H₂O (1 mL) were added methylboronic acid (140 mg, 2.4 mmol), Cs₂CO₃ (290 mg, 0.90 mmol) and Pd(dppf)Cl₂ (22 mg, 0.030 mmol). The resulting mixture was heated to 100° C. overnight. After cooling to room temperature, solvent was removed and the residue was purified by flash column chromatography (silica gel, ethyl acetate in petroleum ether) to give crude product which was further purified by prep-HPLC to afford tert-butyl (2R,5S)-4-(7-(4-cyano-6-methylpyridin-2-yl)-5-(trifluoromethyl)-7H-pyrrolo[2,3-d]pyrimidin-4-yl)-2,5-dimethylpiperazine-1-carboxylate (40 mg, 26%) as a white solid. LC/MS ESI (m/z): 516 (M+H)⁺. ¹H NMR (400 MHz, CDCl₃) δ 9.11 (s, 1H), 8.65 (s, 1H), 8.57 (s, 1H), 7.34 (s, 1H), 4.57 (s, 1H), 4.38 (s, 1H), 3.81-3.71 (m, 2H), 3.64 (dd, J=13.4, 3.5 Hz, 1H), 3.47 (d, J=13.3 Hz, 1H), 2.67 (s, 3H), 1.49 (s, 9H), 1.15 (d, J=6.6 Hz, 3H), 1.11 (d, J=6.8 Hz, 3H).

### Example 162. Synthesis of tert-butyl (2R,5S)-4-(7-(4-chloropyridin-2-yl)-5-(difluoromethoxy)-7H-pyrrolo[2,3-d]pyrimidin-4-yl)-2,5-dimethylpiperazine-1-carboxylate (Compound 521)

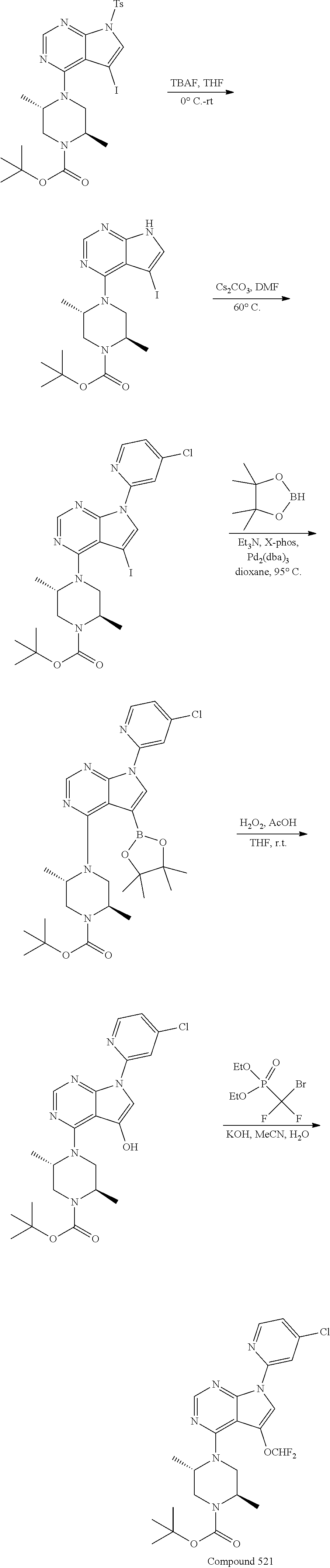

### Step 1. tert-Butyl (2R,5S)-4-(5-iodo-7H-pyrrolo[2,3-d]pyrimidin-4-yl)-2,5-dimethylpiperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (3.5 g, 5.7 mmol) in THE (20 mL) at 0° C. was added TBAF (23 mL, 1.0M in THF). After stirring at room temperature under N₂ for 1 h, the reaction mixture was quenched with ice water and extracted twice with EtOAc. The combined organic layers were washed with aqueous saturated NH₄Cl, dried over Na₂SO₄, filtered, and concentrated under reduced pressure. The residue was purified by flash column chromatography (silica gel, 0˜50%, ethyl acetate in petroleum ether) to obtain tert-butyl (2R,5S)-4-(5-iodo-7H-pyrrolo[2,3-d]pyrimidin-4-yl)-2,5-dimethylpiperazine-1-carboxylate (1.5 g, 57%) as a white solid. LC/MS ESI (m/z): 458 (M+H)⁺.

### Step 2. tert-Butyl (2R,5S)-4-(7-(4-chloropyridin-2-yl)-5-iodo-7H-pyrrolo[2,3-d]pyrimidin-4-yl)-2,5-dimethylpiperazine-1-carboxylate

To the solution of tert-butyl (2R,5S)-4-(5-iodo-7H-pyrrolo[2,3-d]pyrimidin-4-yl)-2,5-dimethylpiperazine-1-carboxylate (500 mg, 1.1 mmol) in DMF (5 mL) were added Cs₂CO₃ (2.0 g, 6.4 mmol) and 4-chloro-2-fluoropyridine (0.32 mL, 3.2 mmol) respectively. The resulting reaction mixture was stirred at 60° C. under N2 overnight. After cooling to room temperature, the reaction mixture was filtered and the filtrate was partitioned between EtOAc and water. The aqueous layer was extracted twice with EtOAc. The combined organic layers were washed with brine, dried over Na₂SO₄, filtered, and concentrated under reduced pressure. The residue was purified by flash column chromatography (silica gel, 0˜20%, ethyl acetate in petroleum ether) to obtain tert-butyl (2R,5S)-4-(7-(4-chloropyridin-2-yl)-5-iodo-7H-pyrrolo[2,3-d]pyrimidin-4-yl)-2,5-dimethylpiperazine-1-carboxylate (330 mg, 53%) as a white solid. LC/MS ESI (m/z): 569 (M+H)⁺.

### Step 3. tert-Butyl (2R,5S)-4-(7-(4-chloropyridin-2-yl)-5-(4,4,5,5-tetramethyl-1,3,2-dioxaborolan-2-yl)-7H-pyrrolo[2,3-d]pyrimidin-4-yl)-2,5-dimethylpiperazine-1-carboxylate

To the solution of tert-butyl (2R,5S)-4-(7-(4-chloropyridin-2-yl)-5-iodo-7H-pyrrolo[2,3-d]pyrimidin-4-yl)-2,5-dimethylpiperazine-1-carboxylate (420 mg, 0.74 mmol) in dioxane (5 mL) were added X-Phos (35 mg, 0.74 mmol), Pd₂(dba)₃ (68 mg, 0.74 mmol), TEA (0.51 mL, 3.7 mmol) and 4,4,5,5-tetramethyl-1,3,2-dioxaborolane (0.43 mL, 3.0 mmol) respectively. The resultant reaction mixture was stirred at 95° C. under N2 overnight. After cooling to room temperature, the reaction mixture was quenched with ice water and extracted twice with EtOAc. The combined organic layers were washed with saturated NaCl, dried over Na₂SO₄, filtered, and concentrated under reduced pressure. The crude product was purified by column chromatography on silica gel (0˜20% ethyl acetate in petroleum ether) to obtain tert-butyl (2R,5S)-4-(7-(4-chloropyridin-2-yl)-5-(4,4,5,5-tetramethyl-1,3,2-dioxaborolan-2-yl)-7H-pyrrolo[2,3-d]pyrimidin-4-yl)-2,5-dimethylpiperazine-1-carboxylate (270 mg, 64%) as a white solid. LC/MS(ESI) m/z: 569 (M+H)⁺.

### Step 4. tert-Butyl (2R,5S)-4-(7-(4-chloropyridin-2-yl)-5-hydroxy-7H-pyrrolo[2,3-d]pyrimidin-4-yl)-2,5-dimethylpiperazine-1-carboxylate

To the solution of tert-butyl (2R,5S)-4-(7-(4-chloropyridin-2-yl)-5-(4,4,5,5-tetramethyl-1,3,2-dioxaborolan-2-yl)-7H-pyrrolo[2,3-d]pyrimidin-4-yl)-2,5-dimethylpiperazine-1-carboxylate (50 mg, 0.088 mmol) in THE (3 mL) were added AcOH (0.50 mL, 8.7 mmol) and H₂O₂ (0.5 mL) respectively. After stirring at room temperature overnight, the reaction mixture was quenched with ice water and extracted twice with EtOAc. The combined organic layers were dried over Na₂SO₄, filtered, and concentrated under reduced pressure to afford tert-butyl (2R,5S)-4-(7-(4-chloropyridin-2-yl)-5-hydroxy-7H-pyrrolo[2,3-d]pyrimidin-4-yl)-2,5-dimethylpiperazine-1-carboxylate (11 mg, 28%) as a yellow solid. LC/MS(ESI) m/z: 459 (M+H)⁺.

### Step 5. tert-Butyl (2R,5S)-4-(7-(4-chloropyridin-2-yl)-5-(difluoromethoxy)-7H-pyrrolo[2,3-d]pyrimidin-4-yl)-2,5-dimethylpiperazine-1-carboxylate

To a solution of tert-butyl (2R,5S)-4-(7-(4-chloropyridin-2-yl)-5-hydroxy-7H-pyrrolo[2,3-d]pyrimidin-4-yl)-2,5-dimethylpiperazine-1-carboxylate (100 mg, 0.22 mmol) in MeCN (5 mL) were added a solution of KOH (250 mg, 4.5 mmol) in H₂O (5 mL) and diethyl (bromodifluoromethyl)phosphonate (70 mg, 0.26 mmol) respectively. The resulting reaction mixture was stirred at room temperature for 1 h and then concentrated under reduced pressure. The crude product was purified by column chromatography on silica gel (0˜20% ethyl acetate in petroleum ether) and prep-HPLC to obtain tert-butyl (2R,5S)-4-(7-(4-chloropyridin-2-yl)-5-(difluoromethoxy)-7H-pyrrolo[2,3-d]pyrimidin-4-yl)-2,5-dimethylpiperazine-1-carboxylate (6.6 mg, 5.9%) as a white solid. LC/MS(ESI) (m/z): 509 (M+H)⁺. ¹H NMR (400 MHz, CDCl₃) δ 9.00 (d, J=1.6 Hz, 1H), 8.44 (s, 1H), 8.35 (d, J=5.3 Hz, 1H), 7.95 (s, 1H), 7.19 (dd, J=5.3, 1.8 Hz, 1H), 6.52 (t, J=73.8 Hz, 1H), 4.86-4.76 (m, 1H), 4.56-4.48 (m, 0.5H, rotamer), 4.36-4.27 (m, 0.5H, rotamer), 4.11-4.05 (m, 1H), 3.84-3.73 (m, 1H), 3.66-3.59 (m, 1H), 3.50-3.41 (m, 1H), 1.50 (s, 9H), 1.27-1.25 (m, 3H), 1.19-1.15 (m, 3H).

### Example 163. Synthesis of tert-butyl (S)-4-(7-(3-chlorophenyl)-5-(cyclopropyl(methyl)amino)-7H-pyrrolo[2,3-d]pyrimidin-4-yl)-3-methylpiperazine-1-carboxylate (Compound 522)

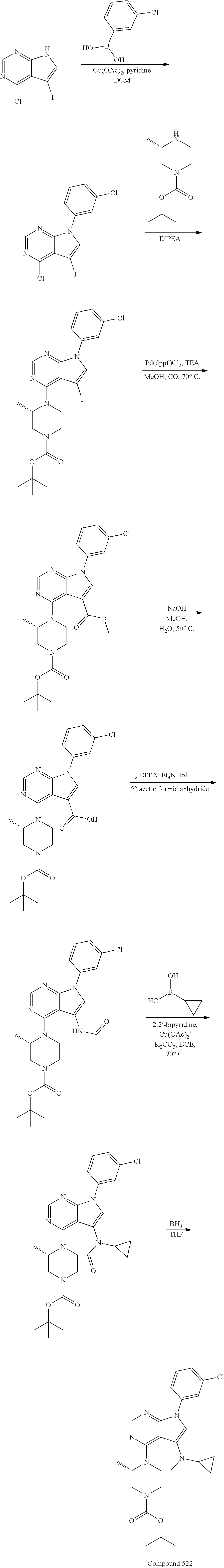

### Step 1. 4-Chloro-7-(3-chlorophenyl)-5-iodo-7H-pyrrolo[2,3-d]pyrimidine

To a mixture of 4-chloro-5-iodo-7H-pyrrolo[2,3-d]pyrimidine (10 g, 36 mmol), (3-chlorophenyl)boronic acid (11 g, 72 mmol) and Cu(OAc)₂ (10 g, 55 mmol) in DCM (300 mL) were added pyridine (18 mL, 220 mmol) and 4 Å MS. The mixture was stirred under O₂ at room temperature for 2 days. It was then treated with aqueous NH₄OH (25%) and filtered through Celite. The filtrate was washed with water, dried over Na₂SO₄ and concentrated to dryness. The crude solid product was triturated with EtOAc/petroleum ether (1:5, v/v) to give 4-chloro-7-(3-chlorophenyl)-5-iodo-7H-pyrrolo[2,3-d]pyrimidine (12 g, 75% purity, 64%) as a brown solid. LC/MS ESI (m/z): 390 (M+H)⁺.

### Step 2. tert-Butyl (S)-4-(7-(3-chlorophenyl)-5-iodo-7H-pyrrolo[2,3-d]pyrimidin-4-yl)-3-methylpiperazine-1-carboxylate

A mixture of 4-chloro-7-(3-chlorophenyl)-5-iodo-7H-pyrrolo[2,3-d]pyrimidine (3.0 g, ˜75% purity, 5.8 mmol) and tert-butyl (S)-3-methylpiperazine-1-carboxylate (3.0 g, 15 mmol) in DIPEA (6.5 mL, 39 mmol) was stirred at 140° C. for 2.5 h. DIPEA was removed by rotary evaporation. The residue was purified by flash column chromatography (silica gel, 0˜16% EtOAc in petroleum ether) to provide tert-butyl (S)-4-(7-(3-chlorophenyl)-5-iodo-7H-pyrrolo[2,3-d]pyrimidin-4-yl)-3-methylpiperazine-1-carboxylate (2.6 g, 82%) as a light brown foam. LC/MS ESI (m/z): 554 (M+H)⁺.

### Step 3. Methyl (S)-4-(4-(tert-butoxycarbonyl)-2-methylpiperazin-1-yl)-7-(3-chlorophenyl)-7H-pyrrolo[2,3-d]pyrimidine-5-carboxylate

To a solution of tert-butyl (S)-4-(7-(3-chlorophenyl)-5-iodo-7H-pyrrolo[2,3-d]pyrimidin-4-yl)-3-methylpiperazine-1-carboxylate (400 mg, 0.72 mmol) in MeOH (10 mL) were added TEA (0.20 mL, 1.4 mmol) and Pd(dppf)Cl₂ (53 mg, 0.070 mmol). The resulting mixture was stirred under CO at 70° C. overnight. After cooling to room temperature, the reaction mixture was filtered and concentrated in vacuo to give the crude product which was purified by flash column chromatography (silica gel, 0˜30% EtOAc in petroleum ether) to afford methyl (S)-4-(4-(tert-butoxycarbonyl)-2-methylpiperazin-1-yl)-7-(3-chlorophenyl)-7H-pyrrolo[2,3-d]pyrimidine-5-carboxylate (270 mg, 77%) as a light yellow solid. LC/MS (ESI) (m/z): 486 (M+H)⁺.

### Step 4. (S)-4-(4-(tert-Butoxycarbonyl)-2-methylpiperazin-1-yl)-7-(3-chlorophenyl)-7H-pyrrolo[2,3-d]pyrimidine-5-carboxylic acid

To a solution of methyl (S)-4-(4-(tert-butoxycarbonyl)-2-methylpiperazin-1-yl)-7-(3-chlorophenyl)-7H-pyrrolo[2,3-d]pyrimidine-5-carboxylate (270 mg, 0.60 mmol) in MeOH (8 m1) was added a solution of NaOH (90 mg, 2.2 mmol) in H₂O (2 mL). The resulting mixture was stirred at 50° C. overnight. After cooling to room temperature, the reaction was quenched with 1N HCl to pH 4-5 and extracted twice with DCM/IPA (85:15 v/v). The combined organic layers were dried over anhydrous Na₂SO₄, filtered and concentrated to afford (S)-4-(4-(tert-butoxycarbonyl)-2-methylpiperazin-1-yl)-7-(3-chlorophenyl)-7H-pyrrolo[2,3-d]pyrimidine-5-carboxylic acid (200 mg, 76%) as a white solid which was used in the next step directly. LC/MS (ESI) (m/z): 472 (M+H)⁺.

### Step 5. tert-Butyl (S)-4-(5-(azidocarbonyl)-7-(3-chlorophenyl)-7H-pyrrolo[2,3-d]pyrimidin-4-yl)-3-methylpiperazine-1-carboxylate (S)-4-(4-(tert-butoxycarbonyl)-2-methylpiperazin-1-yl)-7-(3-chlorophenyl)-7H-pyrrolo[2,3-d]pyrimidine-5-carboxylate

To a solution of (S)-4-(4-(tert-butoxycarbonyl)-2-methylpiperazin-1-yl)-7-(3-chlorophenyl)-7H-pyrrolo[2,3-d]pyrimidine-5-carboxylic acid (200 mg, 0.42 mmol) in toluene (10 mL) were added TEA (0.20 mL, 1.3 mmol) and DPPA (230 mg, 0.84 mmol). The resulting mixture was stirred at room temperature for 2 h and then treated with acetic formic anhydride (3 mL, prepared from acetic anhydride and formic acid). The reaction mixture was stirred at 60° C. for 2 h. After cooling to room temperature, the reaction was quenched with NaHCO₃ (aq.) and extracted with EtOAc. The organic layer was dried over Na₂SO₄ and concentrated. The residue was purified by flash column chromatography (silica gel, 0˜60% EtOAc in petroleum ether) to afford tert-butyl (S)-4-(7-(3-chlorophenyl)-5-(N-formylformamido)-7H-pyrrolo[2,3-d]pyrimidin-4-yl)-3-methylpiperazine-1-carboxylate (70 mg, 37%) as a light yellow solid. LC/MS (ESI) (m/z): 471 (M+H)⁺.

### Step 6. tert-Butyl (S)-4-(7-(3-chlorophenyl)-5-(N-formylformamido)-7H-pyrrolo[2,3-d]pyrimidin-4-yl)-3-methylpiperazine-1-carboxylate

To a solution of tert-butyl (S)-4-(7-(3-chlorophenyl)-5-formamido-7H-pyrrolo[2,3-d]pyrimidin-4-yl)-3-methylpiperazine-1-carboxylate (70 mg, 0.15 mmol) in DCE (10 mL) were added cyclopropylboronic acid (39 mg, 0.45 mmol), Cu(OAc)₂ (55 mg, 0.30 mmol), 2,2′-bipyridine (47 mg, 0.30 mmol) and K₂CO₃ (83 mg, 0.60 mmol). The resulting mixture was stirred at 70° C. overnight. After cooling to room temperature, the mixture was diluted with H₂O and extracted twice with EtOAc. The combined organic layers were washed with brine, dried over anhydrous Na₂SO₄, filtered and concentrated. The residue was purified by column chromatography on silica gel (0-70% EtOAc in petroleum ether) to afford tert-butyl (S)-4-(7-(3-chlorophenyl)-5-(N-cyclopropylformamido)-7H-pyrrolo[2,3-d]pyrimidin-4-yl)-3-methylpiperazine-1-carboxylate (36 mg, 47%) as yellow solid. LC/MS (ESI) (m/z): 511 (M+H)⁺.

### Step 7. tert-Butyl (S)-4-(7-(3-chlorophenyl)-5-(cyclopropyl(methyl)amino)-7H-pyrrolo[2,3-d]pyrimidin-4-yl)-3-methylpiperazine-1-carboxylate

To a solution of tert-butyl (S)-4-(7-(3-chlorophenyl)-5-(N-cyclopropylformamido)-7H-pyrrolo[2,3-d]pyrimidin-4-yl)-3-methylpiperazine-1-carboxylate (36 mg, 0.070 mmol) in THF (5 mml) at 0° C. was added BH₃-THF (1.4 mL, 1.0 M in THF). After stirring at 0° C. for 0.5 h under N₂, the reaction was quenched with methanol and partitioned between EtOAc and water. The aqueous layer was extracted twice with EtOAc. The combined organic layers were washed with brine, dried over anhydrous Na₂SO₄, filtered and concentrated in vacuo to give the crude product which was purified by HPLC to obtain the desired product tert-butyl (S)-4-(7-(3-chlorophenyl)-5-(cyclopropyl(methyl)amino)-7H-pyrrolo[2,3-d]pyrimidin-4-yl)-3-methylpiperazine-1-carboxylate (15 mg, 44%) as white solid. LC/MS (ESI) (m/z): 497 (M+H)⁺. ¹H NMR (400 MHz, CD₃OD) δ 8.23 (s, 1H), 7.85 (t, J=2.0 Hz, 1H), 7.68-7.64 (m, 1H), 7.50 (t, J=7.7 Hz, 1H), 7.38-7.31 (m, 1H), 7.26 (s, 1H), 4.95 (s, 1H), 4.08 (br. d, J=12.6 Hz, 2H), 3.87 (d, J=13.1 Hz, 1H), 3.42-3.31 (m, 2H), 3.15-3.00 (m, 1H), 2.87 (s, 3H), 2.38-2.29 (m, 1H), 1.49 (s, 9H), 1.09 (s, 3H), 0.79-0.70 (m, 2H), 0.60-0.42 (m, 2H).

The following compound was prepared by a procedure similar to the synthesis of Compound 522 from the corresponding boronic acid.

| Cmpd | | |
|---|---|---|
| No. | Chemical Name | LCMS and ¹H NMR |
| 526 | tert-butyl (S)-4-(7-(3- | LC/MS ESI (m/z): 488 (M + H)⁺. ¹H NMR (400 MHz, |
| | cyanophenyl)-5- | CDCl₃) δ 8.30 (s, 1H), 8.04-7.98 (m, 2H), 7.56-7.49 (m, |
| | (cyclopropyl(methyl)amino)-7H- | 2H), 6.91-6.89 (m, 1H), 4.87-4.73 (m, 1H), 4.12-3.74 |
| | pyrrolo[2,3-d]pyrimidin-4-yl)-3- | (m, 3H), 3.32-3.15 (m, 2H), 3.01-2.88 (m, 1H), 2.78 (s, |
| | methylpiperazine-1-carboxylate | 3H), 2.19-2.14 (m, 1H), 1.42 (s, 9H), 1.07 (br, 3H), 0.73- |
| | | 0.67 (m, 2H), 0.51-0.41 (m, 2H). |

### Example 164. Synthesis of 2-(methyl-d3)propan-2-yl-1,1,1,3,3,3-d6 (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (Compound 538)

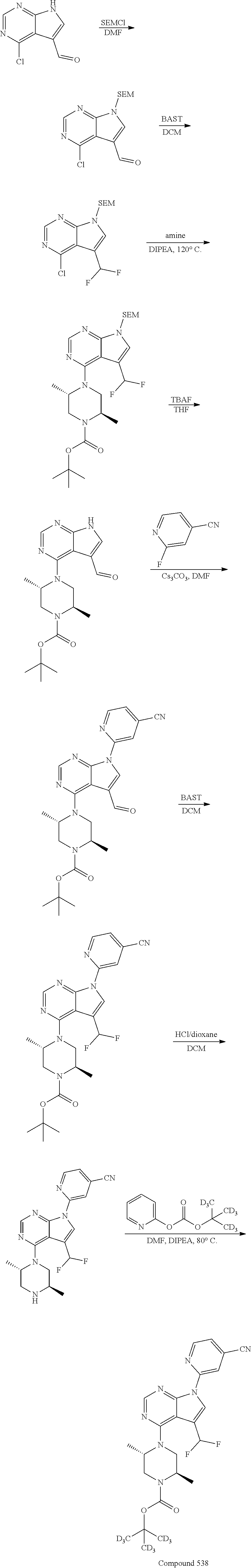

### Step 1. 4-Chloro-7-((2-(trimethylsilyl)ethoxy)methyl)-7H-pyrrolo[2,3-d]pyrimidine-5-carbaldehyde

To a solution of 4-chloro-7H-pyrrolo[2,3-d]pyrimidine-5-carbaldehyde (2.0 g, 11 mmol) in THE (50 mL) at 0° C. was added NaH (790 mg, 33 mmol, 60 wt %) in portions. The resulting mixture was stirred at the same temperature for 20 min and then treated with SEMCl (2.2 g, 13 mmol) dropwise. After stirring at 0° C. for 0.5 h, the reaction mixture was quenched with aqueous NH₄Cl and extracted twice with EtOAc. The combined organic layers were washed with brine, dried over Na₂SO₄, filtered and concentrated. The crude product was purified by flash column chromatography (SiO₂, 0˜40% EtOAc in petroleum ether) to afford 4-chloro-7-((2-(trimethylsilyl)ethoxy)methyl)-7H-pyrrolo[2,3-d]pyrimidine-5-carbaldehyde (2.1 g, 61%) as a white solid. LC/MS ESI (m/z): 312 (M+H)⁺.

### Step 2. 4-Chloro-5-(difluoromethyl)-7-((2-(trimethylsilyl)ethoxy)methyl)-7H-pyrrolo[2,3-d]pyrimidine

To a solution of 4-chloro-7-((2-(trimethylsilyl)ethoxy)methyl)-7H-pyrrolo[2,3-d]pyrimidine-5-carbaldehyde (1.0 g, 3.3 mmol) in DCM (10 mL) at 0° C. was added bis(2-methoxyethyl)aminosulfur trifluoride (BAST) (1.2 mL, 6.6 mmol) dropwise. After stirring at room temperature overnight, the reaction was quenched with aqueous NaHCO₃ and extracted twice with EtOAc. The combined organic layers were washed with brine, dried over Na₂SO₄, filtered and concentrated. The crude product was purified by flash column chromatography (SiO₂, 0˜20% EtOAc in petroleum ether) to afford 4-chloro-5-(difluoromethyl)-7-((2-(trimethylsilyl)ethoxy)methyl)-7H-pyrrolo[2,3-d]pyrimidine (750 mg, 68%) as a white solid. LC/MS ESI (m/z): 334 (M+H)⁺.

### Step 3. tert-Butyl (2R,5S)-4-(5-(difluoromethyl)-7-((2-(trimethylsilyl)ethoxy)methyl)-7H-pyrrolo[2,3-d]pyrimidin-4-yl)-2,5-dimethylpiperazine-1-carboxylate

To a solution of 4-chloro-5-(difluoromethyl)-7-((2-(trimethylsilyl)ethoxy)methyl)-7H-pyrrolo[2,3-d]pyrimidine (750 mg, 2.2 mmol) in DIPEA (5 mL) was added tert-butyl (2R,5S)-2,5-dimethylpiperazine-1-carboxylate (960 mg, 4.5 mmol). The resulting mixture was stirred at 120° C. overnight. After cooling to room temperature, the reaction was concentrated and purified by flash column chromatography (silica gel, 0˜20%, ethyl acetate in petroleum ether) to afford tert-butyl (2R,5S)-4-(5-(difluoromethyl)-7-((2-(trimethylsilyl)ethoxy)methyl)-7H-pyrrolo[2,3-d]pyrimidin-4-yl)-2,5-dimethylpiperazine-1-carboxylate (1.1 g, 95%) as a yellow oil. LC/MS ESI (m/z): 512 (M+H)⁺.

### Step 4. tert-Butyl (2R,5S)-4-(5-formyl-7H-pyrrolo[2,3-d]pyrimidin-4-yl)-2,5-dimethylpiperazine-1-carboxylate

To a solution of tert-butyl (2R,5S)-4-(5-(difluoromethyl)-7-((2-(trimethylsilyl)ethoxy)methyl)-7H-pyrrolo[2,3-d]pyrimidin-4-yl)-2,5-dimethylpiperazine-1-carboxylate (1.1 g, 2.2 mmol) in THF (15 mL) was added TBAF (13 mL, 1.0 M in THF). The resulting mixture was stirred at 60° C. overnight. LCMS showed that the CF₂ group had been hydrolyzed to an aldehyde. After cooling to room temperature, the reaction was partitioned between EtOAc and water. The aqueous layer was extracted twice with EtOAc. The combined organic layers were washed with brine, dried over Na₂SO₄, filtered and concentrated, the residue was purified by flash column chromatography (silica gel, 0˜90%, ethyl acetate in petroleum ether) to afford tert-butyl (2R,5S)-4-(5-formyl-7H-pyrrolo[2,3-d]pyrimidin-4-yl)-2,5-dimethylpiperazine-1-carboxylate (0.76 g, 98%) as a yellow solid. LC/MS ESI (m/z): 360 (M+H)⁺.

### Step 5. tert-Butyl (2R,5S)-4-(7-(4-cyanopyridin-2-yl)-5-formyl-7H-pyrrolo[2,3-d]pyrimidin-4-yl)-2,5-dimethylpiperazine-1-carboxylate

To a solution of tert-butyl (2R,5S)-4-(5-formyl-7H-pyrrolo[2,3-d]pyrimidin-4-yl)-2,5-dimethylpiperazine-1-carboxylate (760 mg, 2.1 mmol) in DMF (10 mL) were added 2-fluoroisonicotinonitrile (520 mg, 4.2 mmol) and Cs₂CO₃ (2.1 g, 6.3 mmol). The resulting mixture was stirred at 50° C. overnight. After cooling to room temperature, the reaction was partitioned between EtOAc and water. The aqueous layer was extracted twice with EtOAc. The combined organic layers were washed with brine, dried over Na₂SO₄, filtered and concentrated, the residue was purified by flash column chromatography (silica gel, 0˜30%, ethyl acetate in petroleum ether) to afford tert-butyl (2R,5S)-4-(7-(4-cyanopyridin-2-yl)-5-formyl-7H-pyrrolo[2,3-d]pyrimidin-4-yl)-2,5-dimethylpiperazine-1-carboxylate (720 mg, 73%) as a yellow solid. LC/MS ESI (m/z): 462 (M+H)⁺.

### Step 6. tert-Butyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate

To a solution of tert-butyl (2R,5S)-4-(7-(4-cyanopyridin-2-yl)-5-formyl-7H-pyrrolo[2,3-d]pyrimidin-4-yl)-2,5-dimethylpiperazine-1-carboxylate (720 mg, 1.6 mmol) in DCM (5 mL) at 0° C. was added BAST (2.8 mL, 16 mmol) dropwise. After stirring at room temperature overnight, the reaction was quenched with NaHCO₃ (aq.). The layers were separated and the aqueous layer was extracted twice with DCM. The combined organic layers were washed with brine, dried over Na₂SO₄, concentrated and purified by flash column chromatography (silica gel, 0-20%, ethyl acetate in petroleum ether) to afford tert-butyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (440 mg, 58%) as a white solid. LC/MS ESI (m/z): 484 (M+H)⁺.

### Step 7. 2-(5-(Difluoromethyl)-4-((2S,5R)-2,5-dimethylpiperazin-1-yl)-7H-pyrrolo[2,3-d]pyrimidin-7-yl)isonicotinonitrile

To a solution of tert-butyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (280 mg, 0.57 mmol) in DCM (10 mL) was added HCl (5.0 mL, 4.0 M in dioxane). After stirring at room temperature for 2 h, the reaction was quenched with NaHCO₃ (aq.). The layers were separated, and the aqueous layer was extracted twice with DCM. The combined organic layers were washed with brine, dried over Na₂SO₄, concentrated and purified by flash column chromatography (silica gel, 0˜10%, methanol in dichloromethane) to afford 2-(5-(difluoromethyl)-4-((2S,5R)-2,5-dimethylpiperazin-1-yl)-7H-pyrrolo[2,3-d]pyrimidin-7-yl)isonicotinonitrile (150 mg, 67%) as a yellow solid. LC/MS ESI (m/z): 384 (M+H)⁺.

### Step 8. 2-(Methyl-d3)propan-2-yl-1,1,1,3,3,3-d6 (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate

To a solution of 2-(methyl-d₃)propan-1,1,1,3,3,3-d₆-2-ol (1.0 g, 12 mmol) in DCM (12 mL) were added DMAP (440 mg, 3.6 mmol) and di(pyridin-2-yl) carbonate (2.59 g, 12 mmol). The resulting mixture was stirred at room temperature for 48 h and used in the next step directly.

To a solution of 2-(5-(difluoromethyl)-4-((2S,5R)-2,5-dimethylpiperazin-1-yl)-7H-pyrrolo[2,3-d]pyrimidin-7-yl)isonicotinonitrile (45 mg, 0.11 mmol) in DMF (2 mL) were added 2-(methyl-d₃)propan-2-yl-1,1,1,3,3,3-d₆ pyridin-2-yl carbonate (0.11 mL, 1.0 M in DCM) and DIPEA (0.50 mL, 0.11 mmol). The resulting mixture was stirred at 80° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by prep-HPLC to afford 2-(methyl-d₃)propan-2-yl-1,1,1,3,3,3-d₆ (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (28 mg, 48%) as a white solid. LC/MS ESI (m/z): 493 (M+H)⁺. ¹H NMR (400 MHz, CDCl₃) δ 9.27 (s, 1H), 8.58 (d, J=4.9 Hz, 1H), 8.50 (s, 1H), 8.44 (s, 1H), 7.38 (d, J=4.9 Hz, 1H), 6.89 (t, J=55.6 Hz, 1H), 4.54-4.46 (m, 1H), 4.40-4.22 (m, 1H), 3.76-3.65 (m, 2H), 3.54 (dd, J=13.5, 3.3 Hz, 1H), 3.45 (d, J=13.1 Hz, 1H), 1.11 (d, J=6.6 Hz, 3H), 1.06 (d, J=6.8 Hz, 3H).

### Example 165. Synthesis of tert-butyl (2R,5S)-4-(7-(4-cyanopyridin-2-yl)-5-(difluoromethoxy)-7H-pyrrolo[2,3-d]pyrimidin-4-yl)-2,5-dimethylpiperazine-1-carboxylate (Compound 523)

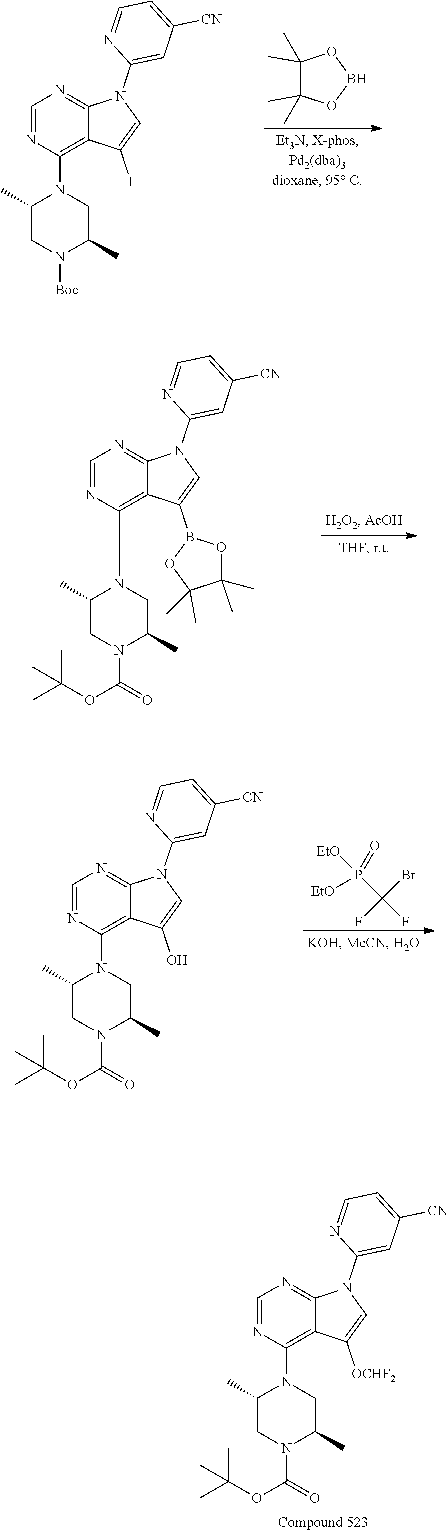

### Step 1. tert-Butyl (2R,5S)-4-(7-(4-cyanopyridin-2-yl)-5-(4,4,5,5-tetramethyl-1,3,2-dioxaborolan-2-yl)-7H-pyrrolo[2,3-d]pyrimidin-4-yl)-2,5-dimethylpiperazine-1-carboxylate

To a solution of tert-butyl (2R,5S)-4-(7-(4-cyanopyridin-2-yl)-5-iodo-7H-pyrrolo[2,3-d]pyrimidin-4-yl)-2,5-dimethylpiperazine-1-carboxylate (950 mg, 1.7 mmol) in dioxane (10 mL) were added Pd₂(dba)₃ (160 mg, 0.17 mmol), X-Phos (81 mg, 0.17 mmol), TEA (1.2 mL, 8.6 mmol) and 4,4,5,5-tetramethyl-1,3,2-dioxaborolane (880 mg, 6.9 mmol) respectively. The resulting reaction mixture was stirred at 95° C. under N2 overnight. After cooling to room temperature, the reaction was quenched with ice water, and extracted with EtOAc. The combined organic layers were dried over Na₂SO₄, filtered, and concentrated. The crude product was purified by column chromatography on silica gel (0-20% ethyl acetate in petroleum ether) to obtain tert-butyl (2R,5S)-4-(7-(4-cyanopyridin-2-yl)-5-(4,4,5,5-tetramethyl-1,3,2-dioxaborolan-2-yl)-7H-pyrrolo[2,3-d]pyrimidin-4-yl)-2,5-dimethylpiperazine-1-carboxylate (700 mg, 70%) as a white solid. LC/MS(ESI) m/z: 560 (M+H)⁺.

### Step 2. tert-Butyl (2R,5S)-4-(7-(4-cyanopyridin-2-yl)-5-hydroxy-7H-pyrrolo[2,3-d]pyrimidin-4-yl)-2,5-dimethylpiperazine-1-carboxylate

To a solution of tert-butyl (2R,5S)-4-(7-(4-cyanopyridin-2-yl)-5-(4,4,5,5-tetramethyl-1,3,2-dioxaborolan-2-yl)-7H-pyrrolo[2,3-d]pyrimidin-4-yl)-2,5-dimethylpiperazine-1-carboxylate (700 mg, 1.2 mmol) in THE (30 mL) were added AcOH (20 mL, 350 mmol) and H₂O₂ (20 mL) respectively. The resulting reaction mixture was stirred at room temperature for 5 h. The reaction was quenched with ice water and extracted with EtOAc. The organic layer was dried over Na₂SO₄, filtered, and concentrated under reduced pressure to afford tert-butyl (2R,5S)-4-(7-(4-cyanopyridin-2-yl)-5-hydroxy-7H-pyrrolo[2,3-d]pyrimidin-4-yl)-2,5-dimethylpiperazine-1-carboxylate (580 mg) as a white solid. LC/MS(ESI) (m/z): 450 (M+H)⁺.

### Step 3. tert-Butyl (2R,5S)-4-(7-(4-cyanopyridin-2-yl)-5-(difluoromethoxy)-7H-pyrrolo[2,3-d]pyrimidin-4-yl)-2,5-dimethylpiperazine-1-carboxylate

To a solution of tert-butyl (2R,5S)-4-(7-(4-cyanopyridin-2-yl)-5-hydroxy-7H-pyrrolo[2,3-d]pyrimidin-4-yl)-2,5-dimethylpiperazine-1-carboxylate (100 mg, 0.22 mmol) in MeCN (5 mL) were added a solution of KOH (250 mg, 0.44 mmol) in H₂O (5 mL) and diethyl (bromodifluoromethyl)phosphonate (70 mg, 0.24 mmol) respectively. The reaction was stirred at room temperature for 2 h after which the resulting mixture was concentrated under reduced pressure. The crude product was purified by column chromatography on silica gel (0˜20% ethyl acetate in petroleum ether) and prep-HPLC to obtain tert-butyl (2R,5S)-4-(7-(4-cyanopyridin-2-yl)-5-(difluoromethoxy)-7H-pyrrolo[2,3-d]pyrimidin-4-yl)-2,5-dimethylpiperazine-1-carboxylate (6.6 mg, 6.0%) as a white solid. LC/MS(ESI) (m/z): 500 (M+H)⁺. ¹H NMR (400 MHz, CDCl₃) δ 9.35 (s, 1H), 8.60 (d, J=5.0 Hz, 1H), 8.44 (s, 1H), 7.96 (s, 1H), 7.39-7.36 (m, 1H), 6.63 (t, J=73.6 Hz, 1H), 4.86-4.77 (m, 1H), 4.57-4.48 (m, 0.5H, rotomer), 4.37-4.25 (m, 0.5H, rotomer), 4.14-4.07 (m, 1H), 3.85-3.73 (m, 1H), 3.66-3.59 (m, 1H), 3.49-3.39 (m, 1H), 1.50 (s, 9H), 1.27-1.24 (m, 3H), 1.18-1.14 (m, 3H).

### Example 166. Synthesis of tert-butyl (2R,5S)-4-(7-(4-cyanopyridin-2-yl)-5-(trifluoromethoxy)-7H-pyrrolo[2,3-d]pyrimidin-4-yl)-2,5-dimethylpiperazine-1-carboxylate (Compound 524)

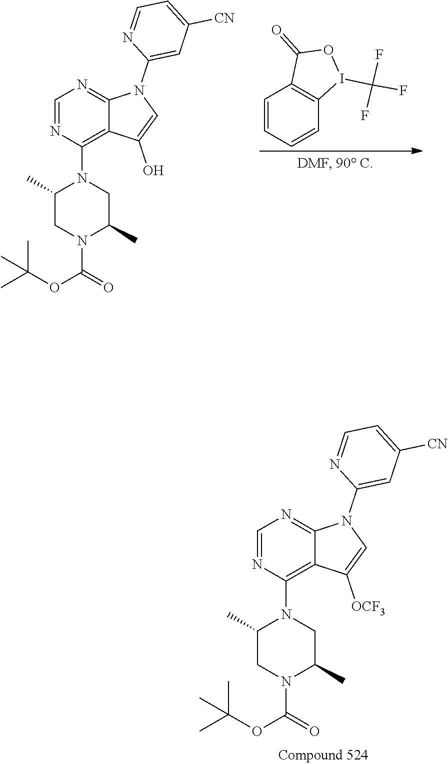 [SMILES: C[C@@H]1CN(c2ncnc3c2c(O)cn3-c2cc(C#N)ccn2)[C@@H](C)CN1C(=O)OC(C)(C)C ; O=C1OI(C(F)(F)F)c2ccccc21 ; C[C@@H]1CN(c2ncnc3c2c(OC(F)(F)F)cn3-c2cc(C#N)ccn2)[C@@H](C)CN1C(=O)OC(C)(C)C]

To a solution of tert-butyl (2R,5S)-4-(7-(4-cyanopyridin-2-yl)-5-hydroxy-7H-pyrrolo[2,3-d]pyrimidin-4-yl)-2,5-dimethylpiperazine-1-carboxylate (100 mg, 0.11 mmol) in DMF (5 mL) was added 1-(trifluoromethyl)-1λ³-benzo[d][1,2]iodaoxol-3 (1H)-one (230 mg, 0.36 mmol). The resulting reaction mixture was stirred at 90° C. under N₂ for 30 min. This procedure was repeated at the same scale and both reactions were worked up together. After cooling to room temperature, the solvent was removed. The residue was purified by column chromatography on silica gel (0-20% ethyl acetate in petroleum ether) and prep-HPLC to obtain tert-butyl (2R,5S)-4-(7-(4-cyanopyridin-2-yl)-5-(trifluoromethoxy)-7H-pyrrolo[2,3-d]pyrimidin-4-yl)-2,5-dimethylpiperazine-1-carboxylate (4.5 mg, 2.0%) as a white solid. LC/MS(ESI) (m/z): 518 (M+H)⁺. ¹H NMR (400 MHz, CDCl₃) δ 9.29 (s, 1H), 8.55 (d, J=5.0 Hz, 1H), 8.39 (s, 1H), 8.07-8.04 (m, 1H), 7.34 (dd, J=5.0, 1.0 Hz, 1H), 4.77-4.69 (m, 1H), 4.50-4.40 (m, 0.5H, rotomer), 4.30-4.19 (m, 0.5H, rotomer), 3.96-3.90 (m, 1H), 3.78-3.67 (m, 1H), 3.62-3.56 (m, 1H), 3.44-3.34 (m, 1H), 1.43 (s, 9H), 1.21-1.19 (m, 3H), 1.09-1.05 (m, 3H).

### Example 167. Synthesis of tert-butyl (2R,5S)-4-(5-(tert-butyl)-7-(4-cyanopyridin-2-yl)-7H-pyrrolo[2,3-d]pyrimidin-4-yl)-2,5-dimethylpiperazine-1-carboxylate (Compound 525)

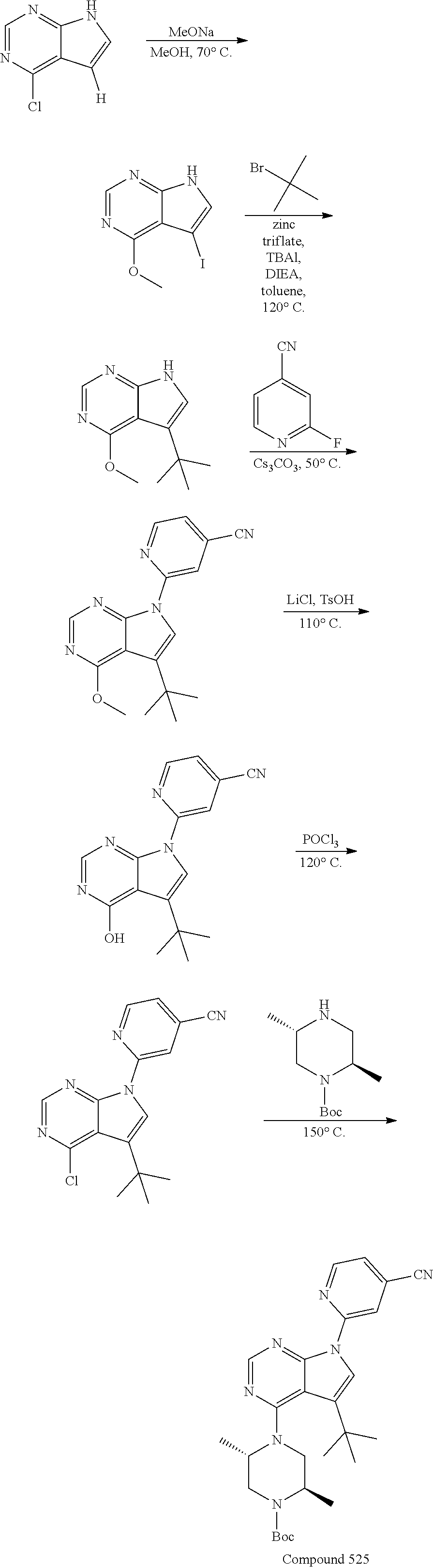

### Step 1. 4-Methoxy-7H-pyrrolo[2,3-d]pyrimidine

To a solution of 4-chloro-7H-pyrrolo[2,3-d]pyrimidine (55 g, 360 mmol) in MeOH (20 mL) was added sodium methanolate (360 mL, 5.0M in methanol). The resulting mixture was stirred at 70° C. overnight. After cooling to room temperature, the reaction mixture was filtered. The filter cake was washed with water and dried under vacuum to afford 4-methoxy-7H-pyrrolo[2,3-d]pyrimidine (50 g, 85%) as a light yellow solid. LC/MS ESI (m/z): 150 (M+H)⁺.

### Step 2. 5-(tert-Butyl)-4-methoxy-7H-pyrrolo[2,3-d]pyrimidine

To a solution of 4-methoxy-7H-pyrrolo[2,3-d]pyrimidine (25 g, 170 mmol) in toluene (100 m1) were added 2-bromo-2-methylpropane (46 g, 340 mmol), zinc triflate (61 g, 170 mmol), TBAI (62 g, 170 mmol) and DIEA (65 g, 500 mmol). The resulting mixture was stirred at 120° C. under N₂ for 1.5 h. After cooling to room temperature, the reaction was quenched with water and extracted twice with EtOAc. The residue was purified by flash column chromatography (silica gel, 0-50%, ethyl acetate in petroleum ether) to afford 5-(tert-butyl)-4-methoxy-7H-pyrrolo[2,3-d]pyrimidine (480 mg, 1.5%) as a white solid. LC/MS ESI (m/z): 206 (M+H)⁺.

### Step 3. 2-(5-(tert-Butyl)-4-methoxy-7H-pyrrolo[2,3-d]pyrimidin-7-yl)isonicotinonitrile

To a solution of 5-(tert-butyl)-4-methoxy-7H-pyrrolo[2,3-d]pyrimidine (45 mg, 0.22 mmol) in DMF (5 mL) were added 2-fluoroisonicotinonitrile (54 mg, 0.44 mmol) and Cs₂CO₃ (360 mg, 1.1 mmol). The resulting mixture was stirred at 50° C. for 2 h. The reaction was quenched with water and extracted twice with EtOAc. The combined organic layers were washed with brine, dried over Na₂SO₄, filtered, and concentrated. The residue was purified by flash column chromatography (silica gel, 0-50%, ethyl acetate in petroleum ether) to afford 2-(5-(tert-butyl)-4-methoxy-7H-pyrrolo[2,3-d]pyrimidin-7-yl)isonicotinonitrile (50 mg, 75%) as a white solid. LC/MS ESI (m/z): 308 (M+H)⁺.

### Step 4. 2-(5-(tert-Butyl)-4-hydroxy-7H-pyrrolo[2,3-d]pyrimidin-7-yl)isonicotinonitrile

To a solution of 2-(5-(tert-butyl)-4-methoxy-7H-pyrrolo[2,3-d]pyrimidin-7-yl)isonicotinonitrile (50 mg, 0.17 mmol) in DMF (5 mL) were added 4-methylbenzenesulfonic acid (280 mg, 1.6 mmol) and LiCl (69 mg, 1.6 mmol). The resulting mixture was stirred at 110° C. for 2 h. After cooling to room temperature, the reaction was quenched with water and extracted twice with EtOAc. The combined organic layers were washed with brine, dried over Na₂SO₄, filtered, and concentrated. The residue was purified by flash column chromatography (silica gel, 0˜100%, ethyl acetate in petroleum ether) to afford 2-(5-(tert-butyl)-4-hydroxy-7H-pyrrolo[2,3-d]pyrimidin-7-yl)isonicotinonitrile (40 mg, 82%) as a yellow solid. LC/MS ESI (m/z): 294 (M+H)⁺.

### Step 5. 2-(5-(tert-Butyl)-4-chloro-7H-pyrrolo[2,3-d]pyrimidin-7-yl)isonicotinonitrile

A solution of 2-(5-(tert-butyl)-4-hydroxy-7H-pyrrolo[2,3-d]pyrimidin-7-yl)isonicotinonitrile (40 mg, 0.14 mmol) in POCl₃ (5 mL) was stirred at 120° C. under N₂ overnight. The solvent was removed. The residue was diluted with EtOAc and washed with saturated NaHCO₃. The aqueous layer was extracted twice with EtOAc. The combined organic layers were dried over Na₂SO₄, filtered and concentrated. The residue was purified by flash column chromatography (silica gel, 0˜30% EtOAc in petroleum ether) to afford 2-(5-(tert-butyl)-4-chloro-7H-pyrrolo[2,3-d]pyrimidin-7-yl)isonicotinonitrile (22 mg, 45%) as a light yellow solid. LC/MS ESI (m/z): 312 (M+H)⁺.

### Step 6. tert-Butyl (2R,5S)-4-(5-(tert-butyl)-7-(4-cyanopyridin-2-yl)-7H-pyrrolo[2,3-d]pyrimidin-4-yl)-2,5-dimethylpiperazine-1-carboxylate

The mixture of 2-(5-(tert-butyl)-4-chloro-7H-pyrrolo[2,3-d]pyrimidin-7-yl)isonicotinonitrile (22 mg, 0.07 mmol) and tert-butyl (2R,5S)-2,5-dimethylpiperazine-1-carboxylate (61 mg, 0.29 mmol) was stirred at 150° C. under N₂ for 3 h. After cooling to room temperature, the reaction mixture was purified by flash column chromatography (silica gel, 0˜50%, ethyl acetate in petroleum ether) to give a crude product (30 mg), which was further purified by prep-HPLC to afford tert-butyl (2R,5S)-4-(5-(tert-butyl)-7-(4-cyanopyridin-2-yl)-7H-pyrrolo[2,3-d]pyrimidin-4-yl)-2,5-dimethylpiperazine-1-carboxylate (15 mg, 50%) as a white solid. LC/MS ESI (m/z): 490 (M+H)⁺. ¹H NMR (400 MHz, CDCl₃) δ 9.35 (s, 1H), 8.62 (dd, J=5.0, 0.7 Hz, 1H), 8.56 (s, 1H), 8.12 (s, 1H), 7.36 (dd, J=5.0, 1.3 Hz, 1H), 4.37-4.28 (m, 2H), 3.83-3.77 (m, 1H), 3.74-3.66 (m, 2H), 3.22-3.16 (m, 1H), 1.54 (s, 9H), 1.49 (s, 9H), 1.12 (d, J=6.8 Hz, 3H), 1.03 (d, J=6.6 Hz, 3H).

### Example 168. Synthesis of tert-butyl (S)-4-(7-(cis-3-cyanocyclohexyl)-5-cyclopropyl-7H-pyrrolo[2,3-d]pyrimidin-4-yl)-3-methylpiperazine-1-carboxylate (Compound 527)

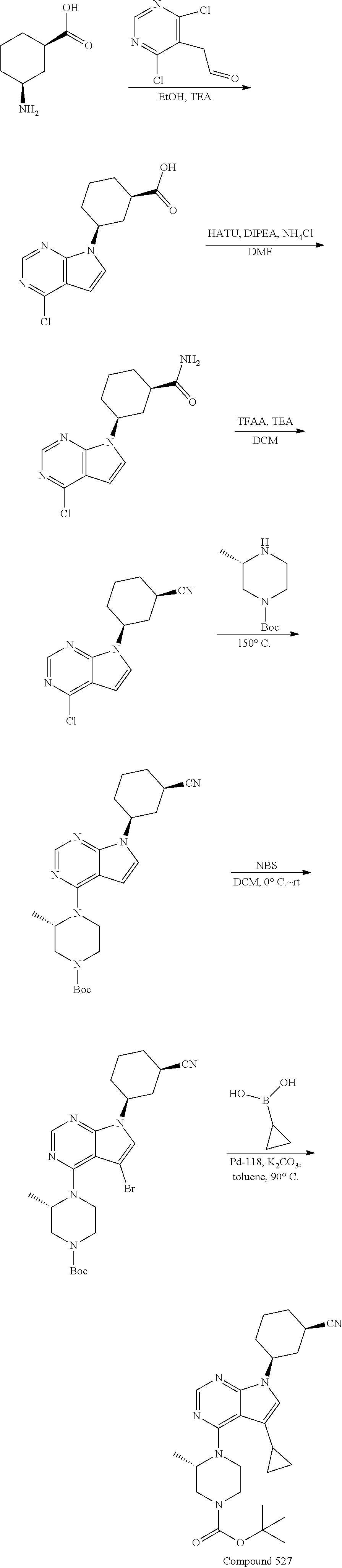

### Step 1. cis-3-(4-Chloro-7H-pyrrolo[2,3-d]pyrimidin-7-yl)cyclohexane-1-carboxylic acid

To a solution of cis-3-aminocyclohexane-1-carboxylic acid (850 mg, 5.9 mmol) in EtOH (10 mL) were added 2-(4,6-dichloropyrimidin-5-yl)acetaldehyde (1.6 g, 8.7 mmol) and TEA (2.9 mL, 21 mmol). The resulting mixture was heated to 80° C. for 3 h under N₂. After cooling to room temperature, the solvent was removed and the residue was purified by flash silica gel column chromatography to afford cis-3-(4-chloro-7H-pyrrolo[2,3-d]pyrimidin-7-yl)cyclohexane-1-carboxylic acid (1.5 g, 95%) as a solid. LC/MS ESI (m/z): 280 (M+H)⁺.

### Step 2. cis-3-(4-Chloro-7H-pyrrolo[2,3-d]pyrimidin-7-yl)cyclohexane-1-carboxamide

To a solution of cis-3-(4-chloro-7H-pyrrolo[2,3-d]pyrimidin-7-yl)cyclohexane-1-carboxylic acid (1.5 g, 5.3 mmol) in DMF (4 mL) were added DIPEA (1.0 g, 12 mmol), ammonium chloride (510 mg, 9.6 mmol) and HATU (3.6 g, 9.6 mmol) respectively. The resulting reaction mixture was stirred at room temperature under N₂ overnight. The reaction mixture was partitioned between EtOAc and water. The organic layer was separated, and the aqueous layer was extracted twice with EtOAc. The combined organic layers were washed with brine, dried over Na₂SO₄, filtered, and concentrated under reduced pressure. The residue was purified by flash column chromatography to afford cis-3-(4-chloro-7H-pyrrolo[2,3-d]pyrimidin-7-yl)cyclohexane-1-carboxamide (960 mg, 59%) as a white solid. LC/MS ESI (m/z): 279 (M+H)⁺.

### Step 3. cis-3-(4-Chloro-7H-pyrrolo[2,3-d]pyrimidin-7-yl)cyclohexane-1-carbonitrile

To a solution of cis-3-(4-chloro-7H-pyrrolo[2,3-d]pyrimidin-7-yl)cyclohexane-1-carboxamide (960 mg, 3.4 mmol) in DCM (10 mL) at 0° C. was added TEA (1.9 mL, 14 mmol) followed by TFAA (0.90 mL, 6.8 mmol) dropwise. The resulting mixture was stirred at the same temperature for 2 h. The reaction was quenched with NaHCO₃ (aq.) and extracted twice with DCM. The combined organic layers were washed with brine, dried over Na₂SO₄, filtered, and concentrated under reduced pressure. The residue was purified by flash column chromatography to afford cis-3-(4-chloro-7H-pyrrolo[2,3-d]pyrimidin-7-yl)cyclohexane-1-carbonitrile (700 mg, 80%) as a solid. LC/MS ESI (m/z): 261 (M+H)⁺.

### Step 4. tert-Butyl (S)-4-(7-(cis-3-cyanocyclohexyl)-7H-pyrrolo[2,3-d]pyrimidin-4-yl)-3-methylpiperazine-1-carboxylate

The mixture of cis-3-(4-chloro-7H-pyrrolo[2,3-d]pyrimidin-7-yl)cyclohexane-1-carbonitrile (680 mg, 2.60 mmol) and tert-butyl (S)-3-methylpiperazine-1-carboxylate (2.6 g, 13 mmol) was stirred at 150° C. for 2 h. After cooling to room temperature, the reaction mixture was purified by column chromatography on silica gel (0˜100% EtOAc in petroleum ether) to give tert-butyl (S)-4-(7-(cis-3-cyanocyclohexyl)-7H-pyrrolo[2,3-d]pyrimidin-4-yl)-3-methylpiperazine-1-carboxylate (800 mg, 72%) as a white solid. LC/MS ESI (m/z): 425 (M+H)⁺.

### Step 5. tert-Butyl (S)-4-(5-bromo-7-(cis-3-cyanocyclohexyl)-7H-pyrrolo[2,3-d]pyrimidin-4-yl)-3-methylpiperazine-1-carboxylate

To a solution of tert-butyl (S)-4-(7-(cis-3-cyanocyclohexyl)-7H-pyrrolo[2,3-d]pyrimidin-4-yl)-3-methylpiperazine-1-carboxylate (800 mg, 1.9 mmol) in DCM (50 mL) at 0° C. was added NBS (350 mg, 2.0 mmol) slowly. The resulting mixture was stirred at room temperature for 2 h. The reaction mixture was partitioned between water and DCM. The organic layer was separated, and the aqueous layer was extracted twice with DCM. The combined organic phases were washed with brine, dried over anhydrous Na₂SO₄, filtered, and concentrated. The residue was purified by column chromatography on silica gel (0˜100% EtOAc in petroleum ether) to give tert-butyl (S)-4-(5-bromo-7-(cis-3-cyanocyclohexyl)-7H-pyrrolo[2,3-d]pyrimidin-4-yl)-3-methylpiperazine-1-carboxylate (650 mg, 50%) as a white solid. LC/MS ESI (m/z): 503, 505 (M+H)⁺.

### Step 6. tert-Butyl (S)-4-(7-(cis-3-cyanocyclohexyl)-5-cyclopropyl-7H-pyrrolo[2,3-d]pyrimidin-4-yl)-3-methylpiperazine-1-carboxylate

A suspension of tert-butyl (S)-4-(5-bromo-7-(cis-3-cyanocyclohexyl)-7H-pyrrolo[2,3-d]pyrimidin-4-yl)-3-methylpiperazine-1-carboxylate (100 mg, 0.20 mmol), cyclopropylboronic acid (85 mg, 0.99 mmol), K₂CO₃ (2.7 g, 19 mmol) and Pd(dtbpf)Cl₂ (97 mg, 0.15 mmol) in toluene (2 mL) was stirred at 90° C. under N₂ for 18 h. After cooling to room temperature, the solvent was removed and the residue was purified by column chromatography on silica gel (0˜50% EtOAc in petroleum ether) and prep-HPLC to give tert-butyl (S)-4-(7-(cis-3-cyanocyclohexyl)-5-cyclopropyl-7H-pyrrolo[2,3-d]pyrimidin-4-yl)-3-methylpiperazine-1-carboxylate (15 mg, 16%) as a white solid. LC/MS ESI (m/z): 465 (M+H)⁺. ¹H NMR (400 MHz, CDCl₃) δ 8.35 (s, 1H), 6.67 (s, 1H), 4.82-4.57 (m, 2H), 4.19-3.74 (m, 3H), 3.59-3.45 (m, 1H), 3.40-3.05 (m, 2H), 2.81-2.66 (m, 1H), 2.41 (t, J=12.3 Hz, 1H), 2.20 (d, J=11.2 Hz, 1H), 2.11-1.84 (m, 4H), 1.80-1.53 (m, 3H), 1.49 (s, 9H), 1.21 (d, J=6.5 Hz, 3H), 0.96 (dd, J=8.1, 1.8 Hz, 2H), 0.79-0.57 (m, 2H).

### Example 169. Synthesis of tert-butyl 4-(7-(4-cyanopyridin-2-yl)-5-(trifluoromethyl)-7H-pyrrolo[2,3-d]pyrimidin-4-yl)-3,3-dimethylpiperazine-1-carboxylate (Compound 528)

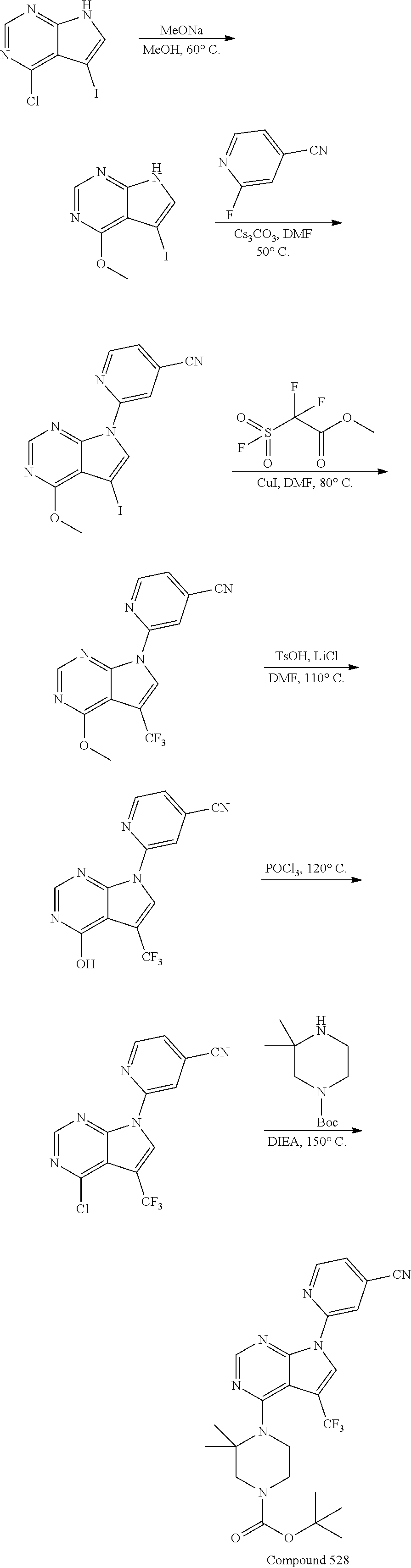

### Step 1. 5-Iodo-4-methoxy-7H-pyrrolo[2,3-d]pyrimidine

To a solution of 4-chloro-5-iodo-7H-pyrrolo[2,3-d]pyrimidine (4.0 g, 14 mmol) in THF (20 mL) was added MeONa (8.0 mL, 4.5 M in MeOH). The resulting mixture was stirred at 60° C. overnight. ⅓ of the solvent was removed and the reaction mixture was poured into ice water. The precipitate was filtered and washed with water and dried under vacuum to afford 5-iodo-4-methoxy-7H-pyrrolo[2,3-d]pyrimidine (3.0 g, 77%) as a white solid. LC/MS ESI (m/z). 276 (M+H).

### Step 2. 2-(5-Iodo-4-methoxy-7H-pyrrolo[2,3-d]pyrimidin-7-yl)isonicotinonitrile

To a solution of 5-iodo-4-methoxy-7H-pyrrolo[2,3-d]pyrimidine (3.0 g, 11 mmol) in DMF (20 mL) were added 2-fluoroisonicotinonitrile (2.7 g, 22 mmol) and Cs₂CO₃ (14 g, 44 mmol). The resulting mixture was stirred at 50° C. overnight. After cooling to room temperature, the reaction mixture was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0-30%, ethyl acetate in petroleum ether) to afford 2-(5-iodo-4-methoxy-7H-pyrrolo[2,3-d]pyrimidin-7-yl)isonicotinonitrile (3.5 g, 85%) as a white-yellow solid. LC/MS ESI (m/z): 378 (M+H)⁺.

### Step 3. 2-(4-Methoxy-5-(trifluoromethyl)-7H-pyrrolo[2,3-d]pyrimidin-7-yl)isonicotinonitrile

To a solution of 2-(5-iodo-4-methoxy-7H-pyrrolo[2,3-d]pyrimidin-7-yl)isonicotinonitrile (3.5 g, 9.3 mmol) in DMF (30 mL) were added CuI (1.8 g, 9.3 mmol) and methyl 2,2-difluoro-2-(fluorosulfonyl)acetate (7.1 g, 37 mmol). The resulting mixture was heated to 80° C. overnight under N₂. After cooling to room temperature, the reaction mixture was partitioned between EtOAc and water. The organic layer was separated, and the aqueous layer was extracted with EtOAc twice. The combined organic layers were washed with brine, dried over Na₂SO₄, filtered, and concentrated. The residue was purified by flash column chromatography (silica gel, 0˜30%, ethyl acetate in petroleum ether) to afford 2-(4-methoxy-5-(trifluoromethyl)-7H-pyrrolo[2,3-d]pyrimidin-7-yl)isonicotinonitrile (1.5 g, 51%) as a light yellow solid. LC/MS ESI (m/z): 320 (M+H)⁺.

### Step 4. 2-(4-Hydroxy-5-(trifluoromethyl)-7H-pyrrolo[2,3-d]pyrimidin-7-yl)isonicotinonitrile

To a solution of 2-(4-methoxy-5-(trifluoromethyl)-7H-pyrrolo[2,3-d]pyrimidin-7-yl)isonicotinonitrile (1.5 g, 4.7 mmol) in DMF (20 mL) were added TsOH (8.1 g, 47 mmol) and LiCl (2.0 g, 47 mmol). The resulting mixture was stirred at 110° C. for 2 h. The reaction was quenched with ice water and extracted with EtOAc twice. The combined organic layers were washed with brine, dried over Na₂SO₄, filtered, and concentrated. The residue was purified by flash column chromatography (silica gel, 0˜60%, ethyl acetate in petroleum ether) to afford 2-(4-hydroxy-5-(trifluoromethyl)-7H-pyrrolo[2,3-d]pyrimidin-7-yl)isonicotinonitrile (0.90 g, 63%) as a light yellow solid. LC/MS ESI (m/z): 306 (M+H)⁺.

### Step 5. 2-(4-Chloro-5-(trifluoromethyl)-7H-pyrrolo[2,3-d]pyrimidin-7-yl)isonicotinonitrile

A solution of 2-(4-hydroxy-5-(trifluoromethyl)-7H-pyrrolo[2,3-d]pyrimidin-7-yl)isonicotinonitrile (0.90 g, 3.0 mmol) in POCl₃ (4 mL) was heated to 120° C. overnight. After cooling to room temperature, the reaction was concentrated. The residue was dissolved in DCM, washed with NaHCO₃ (aq.). The aqueous layer was extracted with DCM twice. The combined organic layers were washed with brine, dried over Na₂SO₄, filtered, and concentrated. The residue was purified by flash column chromatography (silica gel, 0˜40%, ethyl acetate in petroleum ether) to afford 2-(4-chloro-5-(trifluoromethyl)-7H-pyrrolo[2,3-d]pyrimidin-7-yl)isonicotinonitrile (0.70 g, 74%) as a white-yellow solid. LC/MS ESI (m/z): 324 (M+H)⁺.

### Step 6. tert-Butyl 4-(7-(4-cyanopyridin-2-yl)-5-(trifluoromethyl)-7H-pyrrolo[2,3-d]pyrimidin-4-yl)-3,3-dimethylpiperazine-1-carboxylate

To a solution of 2-(4-chloro-5-(trifluoromethyl)-7H-pyrrolo[2,3-d]pyrimidin-7-yl)isonicotinonitrile (300 mg, 0.93 mmol) in DIPEA (5 mL) was added tert-butyl 3,3-dimethylpiperazine-1-carboxylate (2.0 g, 9.3 mmol). The resulting mixture was heated to 150° C. for two days. After cooling to room temperature, the solvent was removed and the residue was purified by flash column chromatography (silica gel, 0˜20%, ethyl acetate in petroleum ether) to give the product. Further purification by prep-HPLC to afford tert-butyl 4-(7-(4-cyanopyridin-2-yl)-5-(trifluoromethyl)-7H-pyrrolo[2,3-d]pyrimidin-4-yl)-3,3-dimethylpiperazine-1-carboxylate (62 mg, 13%) as a white solid. LC/MS ESI (m/z): 502 (M+H)⁺. ¹H NMR (400 MHz, CDCl₃) δ 9.34 (s, 1H), 8.76 (s, 1H), 8.70-8.66 (m, 2H), 7.49 (dd, J=5.0, 1.3 Hz, 1H), 3.68-3.60 (m, 2H), 3.38 (s, 2H), 3.30-3.26 (m, 2H), 1.50 (s, 9H), 1.46 (s, 6H).

The following compound was prepared in a similar fashion to Compound 528 using the corresponding amine.

| Cmpd | | |
|---|---|---|
| No. | Chemical Name | LCMS and ¹H NMR |
| 530 | tert-butyl 4-(7-(4-cyanopyridin- | LC/MS ESI (m/z): 500 (M + H)⁺. ¹H NMR (400 MHz, |
| | 2-yl)-5-(trifluoromethyl)-7H- | CDCl₃) δ 9.33 (s, 1H), 8.68-8.61 (m, 3H), 7.48 (dd, J = |
| | pyrrolo[2,3-d]pyrimidin-4-yl)- | 5.0, 1.3 Hz, 1H), 3.74-3.64 (m, 4H), 3.51-3.30 (m, 2H), |
| | 4,7-diazaspiro[2.5]octane-7- | 1.48 (s, 9H), 0.99-0.91 (m, 2H), 0.80-0.73 (m, 2H). |
| | carboxylate | |

### Example 170. Synthesis of tert-butyl (S)-4-(7-(cis-3-cyanocyclohexyl)-5-(pyrrolidin-1-yl)-7H-pyrrolo[2,3-d]pyrimidin-4-yl)-3-methylpiperazine-1-carboxylate (Compound 529)

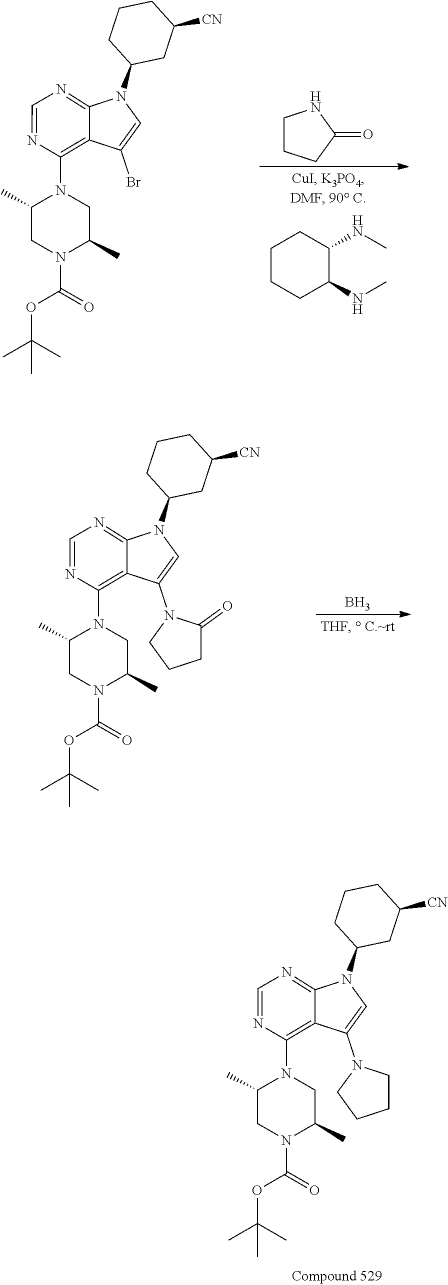

### Step 1. tert-Butyl (S)-4-(7-(cis-3-cyanocyclohexyl)-5-(2-oxopyrrolidin-1-yl)-7H-pyrrolo[2,3-d]pyrimidin-4-yl)-3-methylpiperazine-1-carboxylate

A suspension of tert-butyl (S)-4-(5-bromo-7-(cis-3-cyanocyclohexyl)-7H-pyrrolo[2,3-d]pyrimidin-4-yl)-3-methylpiperazine-1-carboxylate (500 mg, 0.99 mmol), pyrrolidin-2-one (0.23 mL, 3.0 mmol), K₃PO₄ (630 mg, 3.0 mmol), CuI (95 mg, 0.49 mmol) and trans-N¹,N²-dimethylcyclohexane-1,2-diamine (210 mg, 1.5 mmol) in DMF (10 mL) was stirred at 90° C. under N₂ for 18 h. After cooling to room temperature, the reaction mixture was partitioned between EtOAc and water. The organic layer was separated and the aqueous layer was extracted twice with EtOAc. The combined organic layers were washed with brine, dried over anhydrous Na₂SO₄, filtered and concentrated. The residue was purified by column chromatography on silica gel (0˜20% MeOH in DCM) to give tert-butyl (S)-4-(7-(cis-3-cyanocyclohexyl)-5-(2-oxopyrrolidin-1-yl)-7H-pyrrolo[2,3-d]pyrimidin-4-yl)-3-methylpiperazine-1-carboxylate (300 mg, 60%) as a white solid. LC/MS ESI (m/z): 508 (M+H)⁺

### Step 2. tert-Butyl (S)-4-(7-(cis-3-cyanocyclohexyl)-5-(pyrrolidin-1-yl)-7H-pyrrolo[2,3-d]pyrimidin-4-yl)-3-methylpiperazine-1-carboxylate

To a solution of tert-butyl (S)-4-(7-(cis-3-cyanocyclohexyl)-5-(2-oxopyrrolidin-1-yl)-7H-pyrrolo[2,3-d]pyrimidin-4-yl)-3-methylpiperazine-1-carboxylate (100 mg, 0.19 mmol) in dry THF (2 mL) at 0° C. was added BH₃ (0.79 mL, 2.0 M in THF) dropwise. After stirring at room temperature for 3 h, the reaction was quenched with sat. NaHCO₃ and extracted twice with EtOAc. The combined organic phases were washed with brine, dried over anhydrous Na₂SO₄, filtered, and concentrated. The residue was purified by column chromatography on silica gel (0˜20% MeOH in DCM) to give tert-butyl (S)-4-(7-(cis-3-cyanocyclohexyl)-5-(pyrrolidin-1-yl)-7H-pyrrolo[2,3-d]pyrimidin-4-yl)-3-methylpiperazine-1-carboxylate (15 mg, 15%) as a white solid. LC/MS ESI (m/z): 494 (M+H)⁺. ¹H NMR (400 MHz, CDCl₃) δ 8.29 (s, 1H), 6.45 (s, 1H), 5.07-4.87 (m, 1H), 4.75-4.62 (m, 1H), 4.31-3.80 (m, 3H), 3.44-3.17 (m, 2H), 3.14-2.90 (m, 3H), 2.88-2.67 (m, 3H), 2.43 (d, J=12.4 Hz, 1H), 2.20 (d, J=11.7 Hz, 1H), 2.12-1.88 (m, 7H), 1.66-1.53 (m, 3H), 1.48 (s, 9H), 1.11 (d, J=5.4 Hz, 3H).

### Example 171. Synthesis of tert-butyl (2R,5S)-4-(7-(4-cyanopyridin-2-yl)-6-methyl-5-(trifluoromethyl)-7H-pyrrolo[2,3-d]pyrimidin-4-yl)-2,5-dimethylpiperazine-1-carboxylate (Compound 531)

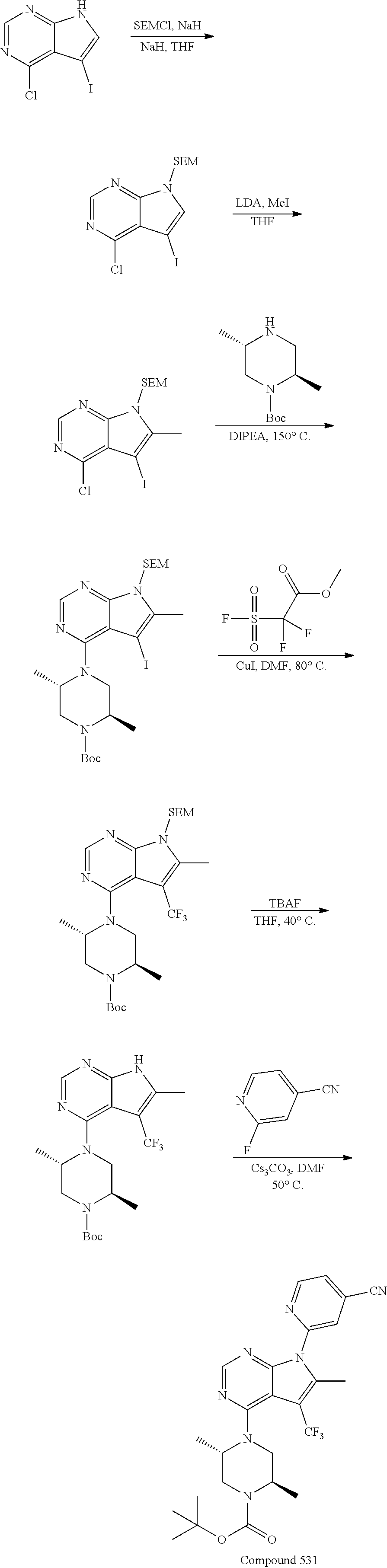

### Step 1. 4-Chloro-5-iodo-7-((2-(trimethylsilyl)ethoxy)methyl)-7H-pyrrolo[2,3-d]pyrimidine

To a suspension of NaH (430 mg, 11 mol, 60 wt %) in THF (20 mL) at 0° C. was added 4-chloro-5-iodo-7H-pyrrolo[2,3-d]pyrimidine (2.0 g, 7.2 mmol) in portions. The resulting mixture was stirred at 0° C. for 20 min after which 2-(trimethylsilyl)ethoxymethyl chloride (1.4 g, 8.6 mmol) was added dropwise. After stirring at room temperature for 3 h, the reaction was quenched with ice water and extracted twice with EtOAc. The combined organic layers were washed with brine, dried over Na₂SO₄, filtered, and concentrated. The residue was purified by flash chromatography (silica gel, 0-30% ethyl acetate in petroleum ether) to afford 4-chloro-5-iodo-7-((2-(trimethylsilyl)ethoxy)methyl)-7H-pyrrolo[2,3-d]pyrimidine (2.0 g, 68%). LC/MS ESI (m/z): 410 (M+H)⁺.

### Step 2. 4-Chloro-5-iodo-6-methyl-7-((2-(trimethylsilyl)ethoxy)methyl)-7H-pyrrolo[2,3-d]pyrimidine

To a solution of 4-chloro-5-iodo-7-((2-(trimethylsilyl)ethoxy)methyl)-7H-pyrrolo[2,3-d]pyrimidine (600 mg, 1.5 mmol) in THE (6 mL) at −70° C. was added LDA (1.5 mL, 2.0M in THF) dropwise. The resulting mixture was stirred at −70° C. for 30 min after which iodomethane (310 mg, 2.2 mmol) was added dropwise. After stirring for 30 min, the reaction was quenched with water and extracted twice with EtOAc. The combined organic layers were washed with brine, dried over Na₂SO₄, filtered, and concentrated. The residue was purified by flash column chromatography (silica gel, 0-30%, ethyl acetate in petroleum ether) to afford 4-chloro-5-iodo-6-methyl-7-((2-(trimethylsilyl)ethoxy)methyl)-7H-pyrrolo[2,3-d]pyrimidine (600 mg, 96%) as a solid. LC/MS ESI (m/z): 424 (M+H)⁺.

### Step 3. tert-Butyl (2R,5S)-4-(5-iodo-6-methyl-7-((2-(trimethylsilyl)ethoxy)methyl)-7H-pyrrolo[2,3-d]pyrimidin-4-yl)-2,5-dimethylpiperazine-1-carboxylate

To a solution of 4-chloro-5-iodo-6-methyl-7-((2-(trimethylsilyl)ethoxy)methyl)-7H-pyrrolo[2,3-d]pyrimidine (600 mg, 1.4 mmol) in DIPEA (5 mL) was added tert-butyl (2R,5S)-2,5-dimethylpiperazine-1-carboxylate (1.5 g, 7.1 mmol). The resulting mixture was stirred at 150° C. for 2 h. After cooling to room temperature, the reaction mixture was purified by flash column chromatography (silica gel, ethyl acetate in petroleum ether) to afford tert-butyl (2R,5S)-4-(5-iodo-6-methyl-7-((2-(trimethylsilyl)ethoxy)methyl)-7H-pyrrolo[2,3-d]pyrimidin-4-yl)-2,5-dimethylpiperazine-1-carboxylate (560 mg, 65%) as a white solid. LC/MS ESI (m/z): 602 (M+H)⁺.

### Step 4. tert-Butyl (2R,5S)-2,5-dimethyl-4-(6-methyl-5-(trifluoromethyl)-7-((2-(trimethylsilyl)ethoxy)methyl)-7H-pyrrolo[2,3-d]pyrimidin-4-yl)piperazine-1-carboxylate

To a solution of tert-butyl (2R,5S)-4-(5-iodo-6-methyl-7-((2-(trimethylsilyl)ethoxy)methyl)-7H-pyrrolo[2,3-d]pyrimidin-4-yl)-2,5-dimethyl piperazine-1-carboxylate (560 mg, 0.93 mmol) in DMF (8 mL) were added CuI (180 mg, 0.93 mmol) and methyl 2,2-difluoro-2-(fluorosulfonyl)acetate (720 mg, 3.7 mmol). The resulting mixture was stirred at 80° C. overnight. After cooling to room temperature, the reaction was partitioned between EtOAc and water. The aqueous layer was extracted twice with EtOAc. The combined organic layers were washed with brine, dried over Na₂SO₄, filtered, and concentrated. The residue was purified by flash column chromatography (silica gel, ethyl acetate in petroleum ether) to afford tert-butyl (2R,5S)-2,5-dimethyl-4-(6-methyl-5-(trifluoromethyl)-7-((2-(trimethylsilyl)ethoxy)methyl)-7H-pyrrolo[2,3-d]pyrimidin-4-yl)piperazine-1-carboxylate (310 mg, 62%) as a light yellow solid. LC/MS ESI (m/z): 544 (M+H)⁺.

### Step 5. tert-Butyl (2R,5S)-2,5-dimethyl-4-(6-methyl-5-(trifluoromethyl)-7H-pyrrolo[2,3-d]pyrimidin-4-yl)piperazine-1-carboxylate

To a solution of tert-butyl (2R,5S)-2,5-dimethyl-4-(6-methyl-5-(trifluoromethyl)-7-((2-(trimethylsilyl)ethoxy)methyl)-7H-pyrrolo[2,3-d]pyrimidin-4-yl)piperazine-1-carboxylate (310 mg, 0.57 mmol) in THE (2 mL) was added TBAF (3.4 mL, 1.0M in THF). The resulting mixture was stirred at 50° C. overnight. After cooling to room temperature, the solvent was removed and the residue was dissolved in DCM. The organics were washed twice with saturated NH₄C1, dried over Na₂SO₄, filtered, and concentrated. The residue was purified by flash column chromatography (silica gel, 0˜50% ethyl acetate in petroleum ether) to afford tert-butyl (2R,5S)-2,5-dimethyl-4-(6-methyl-5-(trifluoromethyl)-7H-pyrrolo[2,3-d]pyrimidin-4-yl)piperazine-1-carboxylate (110 mg, 47%) as an oil. LC/MS ESI (m/z): 414 (M+H)⁺.

### Step 6. tert-Butyl (2R,5S)-4-(7-(4-cyanopyridin-2-yl)-6-methyl-5-(trifluoromethyl)-7H-pyrrolo[2,3-d]pyrimidin-4-yl)-2,5-dimethylpiperazine-1-carboxylate

To a solution of tert-butyl (2R,5S)-2,5-dimethyl-4-(6-methyl-5-(trifluoromethyl)-7H-pyrrolo[2,3-d]pyrimidin-4-yl)piperazine-1-carboxylate (80 mg, 0.19 mmol) in DMF (8 mL) were added Cs₂CO₃ (310 mg, 0.95 mmol) and 2-fluoroisonicotinonitrile (46 mg, 0.38 mmol). The resulting mixture was stirred at 50° C. overnight. After cooling to room temperature, the reaction was partitioned between EtOAc and water. The aqueous layer was extracted twice with EtOAc. The combined organic layers were washed with brine, dried over Na₂SO₄, filtered, and concentrated. The residue was purified by flash column chromatography (silica gel, ethyl acetate in petroleum ether) and prep-HPLC to afford tert-butyl (2R,5S)-4-(7-(4-cyanopyridin-2-yl)-6-methyl-5-(trifluoromethyl)-7H-pyrrolo[2,3-d]pyrimidin-4-yl)-2,5-dimethylpiperazine-1-carboxylate (2.8 mg, 2.8%) as a white solid. LC/MS ESI (m/z): 516 (M+H)⁺. ¹H NMR (400 MHz, CDCl₃) δ 8.83 (d, J=5.0 Hz, 1H), 8.38 (s, 1H), 8.02 (s, 1H), 7.64 (d, J=5.0 Hz, 1H), 4.59-4.42 (m, 1H), 4.41-4.19 (m, 1H), 3.82-3.68 (m, 2H), 3.66-3.60 (m, 1H), 3.44 (d, J=13.4 Hz, 1H), 2.58 (s, 3H), 1.49 (s, 9H), 1.10 (dd, J=12.9, 6.7 Hz, 6H).

### Example 172. Synthesis of tert-butyl (2R,5S)-4-(5-(tert-butyl)-7-(4-cyanopyrimidin-2-yl)-7H-pyrrolo[2,3-d]pyrimidin-4-yl)-2,5-dimethylpiperazine-1-carboxylate (Compound 532)

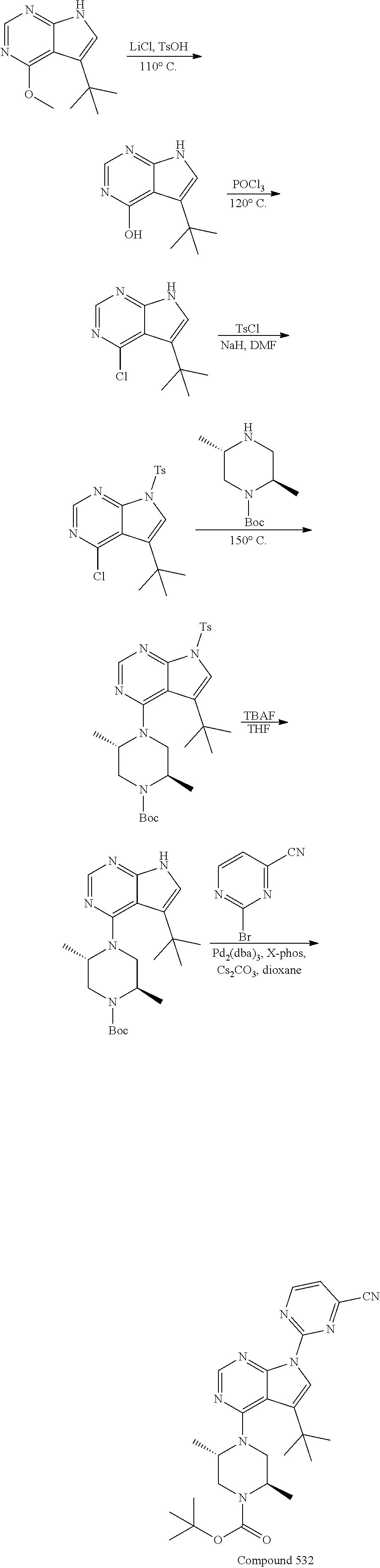

### Step 1. 5-(tert-Butyl)-7H-pyrrolo[2,3-d]pyrimidin-4-ol

To a solution of 5-(tert-butyl)-4-methoxy-7H-pyrrolo[2,3-d]pyrimidine (150 mg, 0.73 mmol) in DMF (5 mL) were added 4-methylbenzenesulfonic acid (1.3 g, 7.3 mmol) and LiCl (310 mg, 7.3 mmol). The resulting mixture was stirred at 110° C. for 2 h. After cooling to room temperature, the reaction was quenched with water and extracted twice with EtOAc. The combined organic layers were washed with brine, dried over Na₂SO₄, filtered, and concentrated. The residue was purified by flash column chromatography (silica gel, 0˜50%, ethyl acetate in petroleum ether) to afford 5-(tert-butyl)-7H-pyrrolo[2,3-d]pyrimidin-4-ol (130 mg, 82%) as a yellow solid. LC/MS ESI (m/z): 192 (M+H)⁺.

### Step 2. 5-(tert-Butyl)-4-chloro-7H-pyrrolo[2,3-d]pyrimidine

A mixture of POCl₃ (15 mL) and 5-(tert-butyl)-7H-pyrrolo[2,3-d]pyrimidin-4-ol (130 mg, 0.68 mmol) was stirred at 120° C. under N₂ atmosphere overnight. After cooling to room temperature, the solvent was removed and the residue was diluted with EtOAc. The mixture was washed with saturated NaHCO₃ and the aqueous layer was extracted twice with EtOAc. The combined organic layers were dried over Na₂SO₄, filtered, and concentrated. The residue was purified by flash column chromatography (silica gel, 0˜30% EtOAc in petroleum ether) to afford 5-(tert-butyl)-4-chloro-7H-pyrrolo[2,3-d]pyrimidine (90 mg, 63%) as a light yellow solid. LC/MS ESI (m/z): 210 (M+H)⁺.

### Step 3. 5-(tert-Butyl)-4-chloro-7-tosyl-7H-pyrrolo[2,3-d]pyrimidine

To a solution of 5-(tert-butyl)-4-chloro-7H-pyrrolo[2,3-d]pyrimidine (90 mg, 0.43 mmol) in DMF (5 mL) were added NaH (21 mg, 0.86 mmol, 60 wt %) and 4-methylbenzenesulfonyl chloride (98 mg, 0.52 mmol) at 0° C. After stirring at room temperature for 2 h, the reaction was quenched with water and extracted twice with EtOAc. The combined organic layers were washed with brine, dried over Na₂SO₄, filtered, and concentrated. The residue was purified by flash column chromatography (silica gel, 0˜25%, ethyl acetate in petroleum ether) to afford 5-(tert-butyl)-4-chloro-7-tosyl-7H-pyrrolo[2,3-d]pyrimidine (120 mg, 82%) as a yellow solid. LC/MS ESI (m/z): 364 (M+H)⁺.

### Step 4. tert-Butyl (2R,5S)-4-(5-(tert-butyl)-7-tosyl-7H-pyrrolo[2,3-d]pyrimidin-4-yl)-2,5-dimethylpiperazine-1-carboxylate

A mixture of 5-(tert-butyl)-4-chloro-7-tosyl-7H-pyrrolo[2,3-d]pyrimidine (120 mg, 0.33 mmol) and tert-butyl (2R,5S)-2,5-dimethylpiperazine-1-carboxylate (350 mg, 1.7 mmol) was stirred at 150° C. under N₂ atmosphere for 3 hours. After cooling to room temperature, the reaction mixture was purified by flash column chromatography (silica gel, 0˜30%, ethyl acetate in petroleum ether) to afford tert-butyl (2R,5S)-4-(5-(tert-butyl)-7-tosyl-7H-pyrrolo[2,3-d]pyrimidin-4-yl)-2,5-dimethylpiperazine-1-carboxylate (150 mg, 82%) as a yellow solid. LC/MS ESI (m/z): 542 (M+H)⁺.

### Step 5. tert-Butyl (2R,5S)-4-(5-(tert-butyl)-7H-pyrrolo[2,3-d]pyrimidin-4-yl)-2,5-dimethylpiperazine-1-carboxylate

To a solution of tert-butyl (2R,5S)-4-(5-(tert-butyl)-7-tosyl-7H-pyrrolo[2,3-d]pyrimidin-4-yl)-2,5-dimethylpiperazine-1-carboxylate (150 mg, 0.28 mmol) in THE (1 mL) was added TBAF (5.0 ml, 1.0M in THF). The resulting mixture was stirred at rt under N₂ atmosphere for 2 hours. The reaction was quenched with water and extracted twice with EtOAc. The combined organic layers were washed with brine, dried over Na₂SO₄, filtered, and concentrated. The residue was purified by flash column chromatography (silica gel, 0˜50%, ethyl acetate in petroleum ether) to afford tert-butyl (2R,5S)-4-(5-(tert-butyl)-7H-pyrrolo[2,3-d]pyrimidin-4-yl)-2,5-dimethylpiperazine-1-carboxylate (100 mg, 92%) as a yellow solid. LC/MS ESI (m/z): 388 (M+H)⁺.

### Step 6. tert-Butyl (2R,5S)-4-(5-(tert-butyl)-7-(4-cyanopyrimidin-2-yl)-7H-pyrrolo[2,3-d]pyrimidin-4-yl)-2,5-dimethylpiperazine-1-carboxylate

To a solution of tert-butyl (2R,5S)-4-(5-(tert-butyl)-7H-pyrrolo[2,3-d]pyrimidin-4-yl)-2,5-dimethylpiperazine-1-carboxylate (55 mg, 0.15 mmol) in dioxane (5 mL) were added 2-bromopyrimidine-4-carbonitrile (72 mg, 0.45 mmol), Pd₂(dba)₃ (60 mg, 0.07 mmol), X-Phos (75 mg, 0.15 mmol) and Cs₂CO₃ (130 mg, 0.45 mmol). The resulting mixture was stirred at 100° C. overnight. After cooling to room temperature, the reaction mixture was partitioned between EtOAc and water. The aqueous layer was extracted twice with EtOAc. The combined organic layers were washed with brine, dried over Na₂SO₄, filtered, and concentrated. The residue was purified by flash column chromatography (silica gel, ethyl acetate in petroleum ether) to give the product which was further purified by prep-HPLC to afford tert-butyl (2R,5S)-4-(5-(tert-butyl)-7-(4-cyanopyrimidin-2-yl)-7H-pyrrolo[2,3-d]pyrimidin-4-yl)-2,5-dimethylpiperazine-1-carboxylate (20 mg, 30%) as a white solid. LC/MS ESI (m/z): 491 (M+H)⁺. ¹H NMR (400 MHz, CDCl₃) δ 9.02 (d, J=4.8 Hz, 1H), 8.59 (s, 1H), 7.89 (s, 1H), 7.38 (d, J=4.8 Hz, 1H), 4.30-4.22 (m, 2H), 3.77-3.70 (m, 1H), 3.67-3.58 (m, 2H), 3.14-3.08 (m, 1H), 1.48 (s, 9H), 1.41 (s, 9H), 1.02 (d, J=6.8 Hz, 3H), 0.97 (d, J=6.7 Hz, 3H).

### Example 173. Synthesis of tert-butyl (2R,5S)-4-(5-(tert-butyl)-7-(6-cyanopyrimidin-4-yl)-7H-pyrrolo[2,3-d]pyrimidin-4-yl)-2,5-dimethylpiperazine-1-carboxylate (Compound 533)

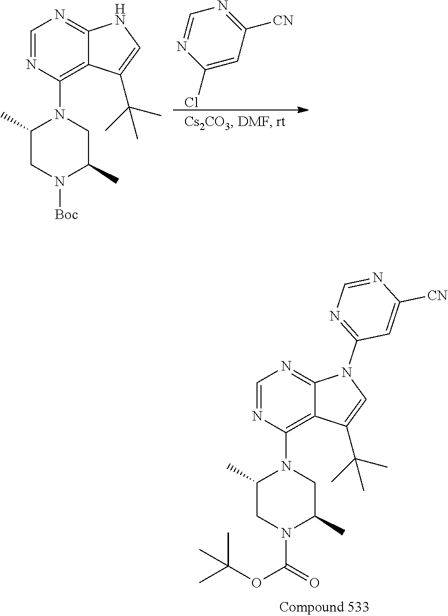

To a solution of tert-butyl (2R,5S)-4-(5-(tert-butyl)-7H-pyrrolo[2,3-d]pyrimidin-4-yl)-2,5-dimethylpiperazine-1-carboxylate (55 mg, 0.15 mmol) in DMF (5 mL) were added Cs₂CO₃ (140 mg, 0.43 mmol) and 6-chloropyrimidine-4-carbonitrile (40 mg, 0.28 mmol). After stirring at room temperature for 3 h, the reaction was filtered. The filtrate was poured into water and extracted twice with EtOAc. The combined organic layers were washed with brine, dried over Na₂SO₄, filtered, and concentrated. The residue was purified by flash column chromatography (silica gel, ethyl acetate in petroleum ether) to give the product which was further purified by prep-HPLC to afford tert-butyl (2R,5S)-4-(5-(tert-butyl)-7-(6-cyanopyrimidin-4-yl)-7H-pyrrolo[2,3-d]pyrimidin-4-yl)-2,5-dimethylpiperazine-1-carboxylate (30 mg, 42%) as a white solid. LC/MS ESI (m/z): 491 (M+H)⁺. ¹H NMR (400 MHz, CDCl₃) δ 9.48 (s, 1H), 9.01 (s, 1H), 8.49 (s, 1H), 8.06 (s, 1H), 4.32-4.23 (m, 2H), 3.75-3.70 (m, 1H), 3.64-3.59 (m, 2H), 3.15-3.10 (m, 1H), 1.46 (s, 9H), 1.42 (s, 9H), 1.00 (t, J=6.6 Hz, 6H).

### Example 174. Synthesis of 2-(methyl-d3)propan-2-yl-1,1,1,3,3,3-d6 (2S,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate (Compound 534)

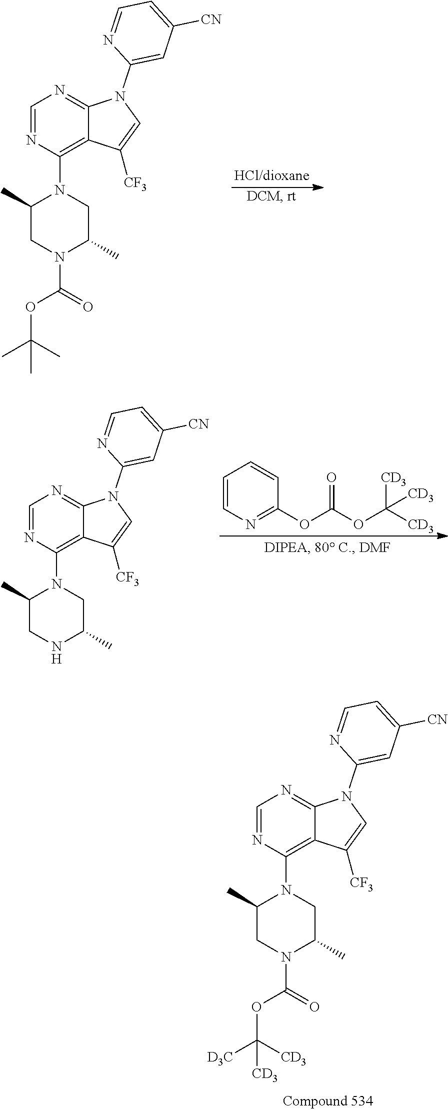 [SMILES: C[C@@H]1CN(C(=O)OC(C)(C)C)[C@@H](C)CN1c1ncnc2c1c(C(F)(F)F)cn2-c1cc(C#N)ccn1 ; "DCM, rt" ; C[C@@H]1CN[C@@H](C)CN1c1ncnc2c1c(C(F)(F)F)cn2-c1cc(C#N)ccn1 ; [2H]C([2H])([2H])C(OC(=O)Oc1ccccn1)(C([2H])([2H])[2H])C([2H])([2H])[2H] ; [2H]C([2H])([2H])C(OC(=O)N1C[C@@H](C)N(c2ncnc3c2c(C(F)(F)F)cn3-c2cc(C#N)ccn2)C[C@@H]1C)(C([2H])([2H])[2H])C([2H])([2H])[2H]]

### Step 1. 2-(4-((2R,5S)-2,5-Dimethylpiperazin-1-yl)-5-(trifluoromethyl)-7H-pyrrolo[2,3-d]pyrimidin-7-yl)isonicotinonitrile

To a solution of tert-butyl (2S,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate (140 mg, 0.28 mmol) in DCM (4 mL) was added HCl (0.50 mL, 4.0 M in dioxane). The resulting mixture was stirred at room temperature for 4 hours. The reaction mixture was basicified with saturated NaHCO₃ and extracted twice with DCM. The combined organic layers were dried over Na₂SO₄, filtered, and concentrated. The residue was purified by flash chromatography (0˜10% MeOH in DCM) to afford 2-(4-((2R,5S)-2,5-dimethylpiperazin-1-yl)-5-(trifluoromethyl)-7H-pyrrolo[2,3-d]pyrimidin-7-yl)isonicotinonitrile (90 mg, 80%) as a white solid. LC/MS ESI (m/z): 402 (M+H)⁺.

### Step 2. 2-(Methyl-d₃)propan-2-yl-1,1,1,3,3,3-d₆ (2S,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate

A mixture of 2-(methyl-d₃)propan-1,1,1,3,3,3-d₆-2-ol (1.0 g, 12 mmol), di(pyridin-2-yl) carbonate (2.6 g, 12 mmol) and DMAP (440 mg, 3.6 mmol) in DCM (30 mL) was stirred at room temperature for 42 h. The reaction mixture was used directly without further manipulation. To a solution of 2-(4-((2R,5S)-2,5-dimethylpiperazin-1-yl)-5-(trifluoromethyl)-7H-pyrrolo[2,3-d]pyrimidin-7-yl)isonicotinonitrile (60 mg, 0.15 mmol) in DMF (5 mL) were added 2-(methyl-d₃)propan-2-yl-1,1,1,3,3,3-d₆ pyridin-2-yl carbonate (0.50 mL, 0.40 M in DCM) and DIPEA (58 mg, 0.45 mmol). The resulting mixture was heated to 80° C. for 1 h. After cooling to room temperature, the reaction was partitioned between EtOAc and water. The aqueous layer was extracted twice with EtOAc. The combined organic layers were washed with brine, dried over Na₂SO₄, filtered, and concentrated. The residue was purified by flash column chromatography (silica gel, 0-25%, ethyl acetate in petroleum ether) and prep-HPLC to afford 2-(methyl-d₃)propan-2-yl-1,1,1,3,3,3-d₆ (2S,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate (50 mg, 64%) as a solid. A sample of 50 mg was further purified by prep-HPLC to afford 30 mg of a white solid. LC/MS ESI (m/z): 511 (M+H)⁺. ¹H NMR (400 MHz, CDCl₃) δ 9.30-9.24 (m, 1H), 8.62-8.56 (m, 2H), 8.50 (s, 1H), 7.41 (dd, J=5.0, 1.3 Hz, 1H), 4.57-4.47 (m, 1H), 4.43-4.17 (m, 1H), 3.75-3.63 (m, 2H), 3.60-3.53 (m, 1H), 3.41 (d, J=13.3 Hz, 1H), 1.09 (d, J=6.6 Hz, 3H), 1.03 (d, J=6.8 Hz, 3H).

The following compound was prepared by a procedure similar to the synthesis of Compound 534 using tert-butyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate.

| Cmpd | | |
|---|---|---|
| No. | Chemical Name | LCMS and ¹H NMR |
| 518 | 2-(methyl-d₃)propan-2-yl- | LC/MS ESI (m/z): 511 (M + H)⁺. ¹H NMR (400 MHz, |
| | 1,1,1,3,3,3-d₆ (2R,5S)-4-(7-(4- | CDCl₃) δ 9.28 (s, 1H), 8.61-8.56 (m, 2H), 8.50 (s, 1H), |
| | cyanopyridin-2-yl)-5- | 7.41 (dd, J = 5.0, 1.3 Hz, 1H), 4.56-4.47 (m, 1H), 4.41- |
| | (trifluoromethyl)-7H- | 4.14 (m, 1H), 3.75-3.64 (m, 2H), 3.59-3.54 (m, 1H), |
| | pyrrolo[2,3-d]pyrimidin-4-yl)- | 3.44-3.39 (m, 1H), 1.09 (d, J = 6.6 Hz, 3H), 1.03 (d, J = |
| | 2,5-dimethylpiperazine-1- | 6.8 Hz, 3H). |
| | carboxylate | |

### Example 175. Synthesis of 1-methylcyclobutyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (Compound 535)

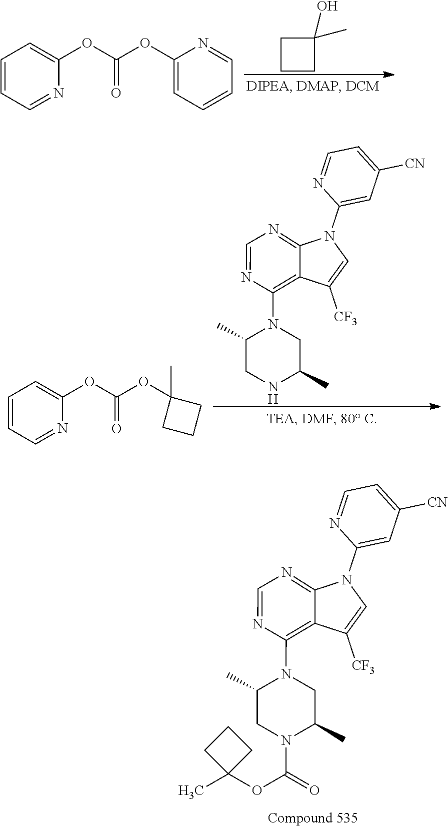

### Step 1. 1-Methylcyclobutyl pyridin-2-yl carbonate

A mixture of 1-methylcyclobutan-1-ol (100 mg, 1.2 mmol), di(pyridin-2-yl) carbonate (240 g, 1.1 mmol), and DMAP (40 mg, 0.33 mmol) in DCM (5 mL) was stirred at room temperature overnight. The reaction mixture quenched with saturated NH₄Cl and extracted twice with DCM. The combined organic layers were washed with brine, dried over Na₂SO₄, filtered and concentrated. The residue was purified by flash chromatography (silica gel, 0-35% ethyl acetate in petroleum ether) to afford 1-methylcyclobutyl pyridin-2-yl carbonate (100 mg, 43%).

### Step 2. 1-Methylcyclobutyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate

A mixture of 1-methylcyclobutyl pyridin-2-yl carbonate (120 mg, 0.56 mmol), 2-(4-((2S,5R)-2,5-dimethylpiperazin-1-yl)-5-(trifluoromethyl)-7H-pyrrolo[2,3-d]pyrimidin-7-yl)isonicotinonitrile (150 mg, 0.37 mmol), TEA (0.10 mL, 0.74 mmol) and DMF (5 mL) was heated to 80° C. overnight. After cooling to room temperature, the reaction mixture was partitioned between EtOAc and water. The aqueous layer was extracted twice with EtOAc and the combined organic layers were washed with brine, dried over Na₂SO₄, filtered, and concentrated. The residue was purified by flash column chromatography (silica gel, 0-25%, ethyl acetate in petroleum ether) and prep-HPLC to afford 1-methylcyclobutyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (60 mg, 31%). LC/MS ESI (m/z): 514 (M+H)⁺. ¹H NMR (400 MHz, CDCl₃) δ 9.27 (s, 1H), 8.64-8.55 (m, 2H), 8.51 (s, 1H), 7.41 (dd, J=5.0, 1.2 Hz, 1H), 4.52 (br. s, 1H), 4.32 (br. s, 1H), 3.77-3.61 (m, 2H), 3.60-3.54 (m, J=12.4 Hz, 1H), 3.41 (d, J=13.4 Hz, 1H), 2.32-2.22 (m, 2H), 2.13-2.04 (m, 2H), 1.79-1.69 (m, 1H), 1.64-1.56 (m, 1H), 1.51 (s, 3H), 1.09 (d, J=6.6 Hz, 3H), 1.05 (d, J=6.8 Hz, 3H).

### Example 176. Synthesis of tert-butyl (2R,5S)-2,5-dimethyl-4-(5-(trifluoromethyl)-7-(4-(trifluoromethyl)pyridin-2-yl)-7H-pyrrolo[2,3-d]pyrimidin-4-yl)piperazine-1-carboxylate (Compound 536)

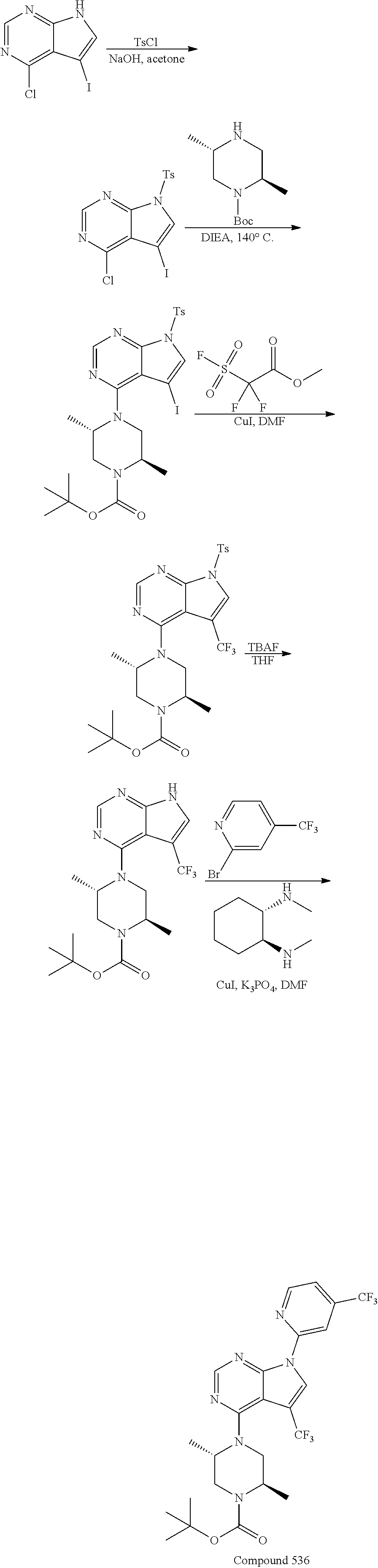

### Step 1. 4-Chloro-5-iodo-7-tosyl-7H-pyrrolo[2,3-d]pyrimidine

To a solution of 4-chloro-5-iodo-7H-pyrrolo[2,3-d]pyrimidine (200 g, 0.71 mol) and 4-methylbenzene-1-sulfonyl chloride (180 g, 0.93 mol) in acetone (2 L) at 0° C. was added dropwise 2.0M NaOH (0.53 L). The reaction was allowed to warm up to room temperature and stirred for 3 hours. The resulting precipitate was collected by filtration, washed with water twice, and dried under vacuum to provide 4-chloro-5-iodo-7-tosyl-7H-pyrrolo[2,3-d]pyrimidine (300 g, 95%) as an off-white solid. LC/MS ESI (m/z): 434 (M+H)⁺.

### Step 2. tert-Butyl (2R,5S)-4-(5-iodo-7-tosyl-7H-pyrrolo[2,3-d]pyrimidin-4-yl)-2,5-dimethylpiperazine-1-carboxylate

To a solution of 4-chloro-5-iodo-7-tosyl-7H-pyrrolo[2,3-d]pyrimidine (20 g, 46 mmol) in DIEA (100 mL) was added tert-butyl (2R,5S)-2,5-dimethylpiperazine-1-carboxylate (20 g, 92 mmol). The resulting mixture was heated to 140° C. for 1 h. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜10%, ethyl acetate in petroleum ether) to afford tert-butyl (2R,5S)-4-(5-iodo-7-tosyl-7H-pyrrolo[2,3-d]pyrimidin-4-yl)-2,5-dimethylpiperazine-1-carboxylate (26 g, 92%) as a yellow solid. LC/MS ESI (m/z): 612 (M+H)⁺.

### Step 3. tert-Butyl (2R,5S)-2,5-dimethyl-4-(7-tosyl-5-(trifluoromethyl)-7H-pyrrolo[2,3-d]pyrimidin-4-yl)piperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (7.0 g, 11 mmol) in DMF (120 mL) were added methyl 2,2-difluoro-2-(fluorosulfonyl)acetate (4.3 mL, 34 mmol) and CuI (2.2 g, 11 mmol). The resulting mixture was heated to 80° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to afford tert-butyl (2R,5S)-2,5-dimethyl-4-(7-tosyl-5-(trifluoromethyl)-7H-pyrrolo[2,3-d]pyrimidin-4-yl)piperazine-1-carboxylate (4.8 g, 75%) as a yellow solid. LC/MS ESI (m/z): 554 (M+H)⁺.

### Step 4. tert-Butyl (2R,5S)-2,5-dimethyl-4-(5-(trifluoromethyl)-7H-pyrrolo[2,3-d]pyrimidin-4-yl)piperazine-1-carboxylate

To a solution of tert-butyl (2R,5S)-2,5-dimethyl-4-(7-tosyl-5-(trifluoromethyl)-7H-pyrrolo[2,3-d]pyrimidin-4-yl)piperazine-1-carboxylate (4.8 g, 8.6 mmol) in THE (50 mL) was added TBAF (52 mL, 1.0 M in THF). After stirring at room temperature for 1 h, the reaction was quenched with water and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford tert-butyl (2R,5S)-2,5-dimethyl-4-(5-(trifluoromethyl)-7H-pyrrolo[2,3-d]pyrimidin-4-yl)piperazine-1-carboxylate (3.2 g, 91%) as a yellow solid. LC/MS ESI (m/z): 400 (M+H)⁺.

### Step 5. tert-Butyl (2R,5S)-2,5-dimethyl-4-(5-(trifluoromethyl)-7-(4-(trifluoromethyl)pyridin-2-yl)-7H-pyrrolo[2,3-d]pyrimidin-4-yl)piperazine-1-carboxylate

To a solution of tert-butyl (2R,5S)-2,5-dimethyl-4-(5-(trifluoromethyl)-7H-pyrrolo[2,3-d]pyrimidin-4-yl)piperazine-1-carboxylate (150 mg, 0.37 mmol) in DMF (5 mL) were added 2-bromo-4-(trifluoromethyl)pyridine (0.09 mL, 0.75 mmol), CuI (72 mg, 0.37 mmol), trans-N¹,N²-dimethylcyclohexane-1,2-diamine (110 mg, 0.75 mmol) and K₃PO₄ (160 mg, 0.75 mmol). The resulting mixture was stirred at 100° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜20%, ethyl acetate in petroleum ether) to give the product which was further purified by prep-HPLC to afford tert-butyl (2R,5S)-2,5-dimethyl-4-(5-(trifluoromethyl)-7-(4-(trifluoromethyl)pyridin-2-yl)-7H-pyrrolo[2,3-d]pyrimidin-4-yl)piperazine-1-carboxylate (33 mg, 16%) as a white solid. LC/MS ESI (m/z): 545 (M+H)⁺. ¹H NMR (400 MHz, CDCl₃) δ 9.27 (s, 1H), 8.68 (m, 2H), 8.58 (s, 1H), 7.49 (d, J=5.1 Hz, 1H), 4.61-4.53 (m, 1H), 4.47-4.26 (m, 1H), 3.82-3.71 (m, 2H), 3.64 (dd, J=13.4, 3.5 Hz, 1H), 3.48 (d, J=13.3 Hz, 1H), 1.50 (s, 9H), 1.15 (d, J=6.6 Hz, 3H), 1.11 (d, J=6.8 Hz, 3H).

The following compounds were prepared by a procedure analogous to the synthesis of Compound 536 from the corresponding aryl halides.

| Cmpd | | |
|---|---|---|
| No. | Chemical Name | LCMS and ¹H NMR |
| 505 | tert-butyl (2R,5S)-4-(7-(4- | LC/MS ESI (m/z): 495 (M + H)⁺. ¹H NMR (400 MHz, |
| | fluoropyridin-2-yl)-5- | CDCl₃) δ 8.69 (dd, J = 10.6, 2.2 Hz, 1H), 8.60-8.57 (m, |
| | (trifluoromethyl)-7H- | 1H), 8.49 (s, 1H), 8.40 (dd, J = 8.5, 5.6 Hz, 1H), 6.97- |
| | pyrrolo[2,3-d]pyrimidin-4-yl)- | 6.93 (m, 1H), 4.54-4.45 (m, 1H), 4.40-4.17 (m, 1H), |
| | 2,5-dimethylpiperazine-1- | 3.73-3.63 (m, 2H), 3.59-3.54 (m, 1H), 3.42-3.37 (m, |
| | carboxylate | 1H), 1.42 (s, 9H), 1.08-1.03 (m, 6H). |
| 513 | tert-butyl (2R,5S)-4-(7-(3- | LC/MS ESI (m/z): 501 (M + H)⁺. ¹H NMR (400 MHz, |
| | cyanophenyl)-5- | CD₃OD) δ 8.46 (s, 1H), 8.27 (m, 2H), 8.12-8.04 (m, 1H), |
| | (trifluoromethyl)-7H- | 7.86-7.70 (m, 2H), 4.64-4.54 (m, 1H), 4.42-4.29 (m, |
| | pyrrolo[2,3-d]pyrimidin-4-yl)- | 1H), 3.82-3.70 (m, 2H), 3.67-3.59 (m, 1H), 3.56-3.48 |
| | 2,5-dimethylpiperazine-1- | (m, 1H), 1.50 (s, 9H), 1.13 (t, J = 6.9 Hz, 6H). |
| | carboxylate | |

### Example 177. Synthesis of tert-butyl (2R,5S)-2,5-dimethyl-4-(5-methyl-7-(4-(trifluoromethyl)pyridin-2-yl)-7H-pyrrolo[2,3-d]pyrimidin-4-yl)piperazine-1-carboxylate (Compound 537)

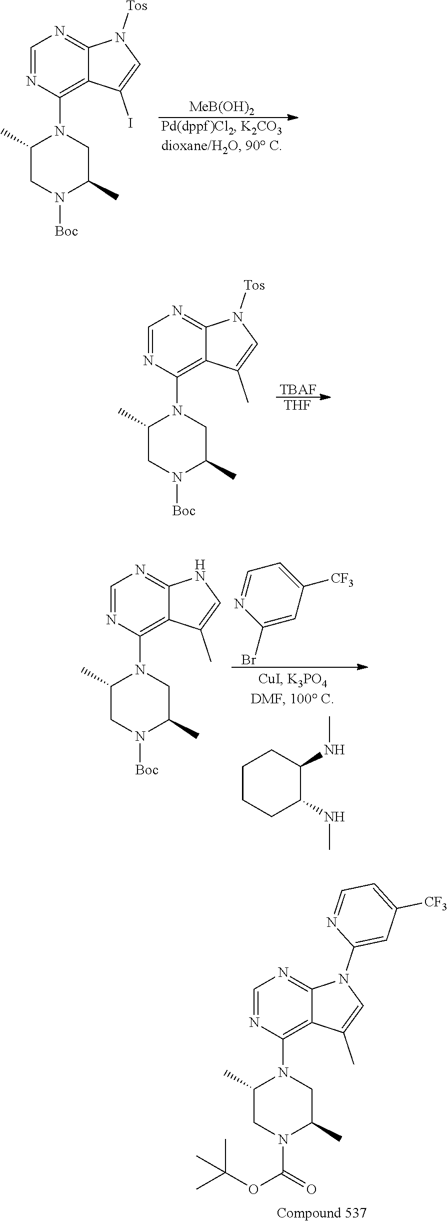

### Step 1. tert-Butyl (2R,5S)-2,5-dimethyl-4-(5-methyl-7-tosyl-7H-pyrrolo[2,3-d]pyrimidin-4-yl)piperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (610 mg, 1.0 mmol) in dioxane (10 mL) and H₂O (1 mL) were added methylboronic acid (600 mg, 10 mmol), K₂CO₃ (410 mg, 3.0 mmol) and Pd(dppf)Cl₂ (73 mg, 0.10 mmol). After stirring at 90° C. overnight under N₂, the reaction mixture was filtered. The filtrate was concentrated and purified by flash column chromatography (silica gel, 0-20%, EtOAc in petroleum ether) to afford tert-butyl (2R,5S)-2,5-dimethyl-4-(5-methyl-7-tosyl-7H-pyrrolo[2,3-d]pyrimidin-4-yl)piperazine-1-carboxylate (300 mg, 60%) as a white-yellow solid. LC/MS ESI (m/z): 500 (M+H)⁺.

### Step 2. tert-Butyl (2R,5S)-2,5-dimethyl-4-(5-methyl-7H-pyrrolo[2,3-d]pyrimidin-4-yl)piperazine-1-carboxylate

To a solution of tert-butyl (2R,5S)-2,5-dimethyl-4-(5-methyl-7-tosyl-7H-pyrrolo[2,3-d]pyrimidin-4-yl)piperazine-1-carboxylate (300 mg, 0.60 mmol) in THE (3 mL) was added TBAF (5.0 mL, 1.0M in THF). After stirring at room temperature overnight under N₂, the reaction was quenched with water and extracted twice with DCM. The combined organic layers were washed with brine and dried over Na₂SO₄, concentrated, and purified by flash column chromatography (silica gel, 0-80%, EtOAc in petroleum ether) to afford tert-butyl (2R,5S)-2,5-dimethyl-4-(5-methyl-7H-pyrrolo[2,3-d]pyrimidin-4-yl)piperazine-1-carboxylate (190 mg, 92%) as a white-yellow solid. LC/MS ESI (m/z): 346 (M+H)⁺.

### Step 3. tert-Butyl (2R,5S)-2,5-dimethyl-4-(5-methyl-7-(4-(trifluoromethyl)pyridin-2-yl)-7H-pyrrolo[2,3-d]pyrimidin-4-yl)piperazine-1-carboxylate

To a solution of tert-butyl (2R,5S)-2,5-dimethyl-4-(5-methyl-7H-pyrrolo[2,3-d]pyrimidin-4-yl)piperazine-1-carboxylate (100 mg, 0.29 mmol) in DMF (10 mL) were added 2-bromo-4-(trifluoromethyl)pyridine (130 mg, 0.58 mmol), trans-N¹,N²-dimethylcyclohexane-1,2-diamine (82 mg, 0.58 mmol), CuI (55 mg, 0.58 mmol) and K₃PO₄ (120 mg, 0.58 mmol). After stirring at 100° C. overnight under N₂, the reaction was cooled to room temperature and partitioned between EtOAc and water. The aqueous layer was extracted twice with EtOAc. The combined organic layers were washed with brine and dried over Na₂SO₄, concentrated and purified by flash column chromatography (silica gel, 0˜20%, EtOAc in petroleum ether) to give the product which was further purified by prep-HPLC to afford tert-butyl (2R,5S)-2,5-dimethyl-4-(5-methyl-7-(4-(trifluoromethyl)pyridin-2-yl)-7H-pyrrolo[2,3-d]pyrimidin-4-yl)piperazine-1-carboxylate (140 mg, 95%) as a white solid. LC/MS ESI (m/z): 491 (M+H)⁺. ¹H NMR (400 MHz, CDCl₃) δ 9.24 (s, 1H), 8.61 (d, J=5.1 Hz, 1H), 8.51 (s, 1H), 7.99 (d, J=1.0 Hz, 1H), 7.36 (d, J=4.6 Hz, 1H), 4.52-4.33 (m, 2H), 3.81-3.73 (m, 2H), 3.67-3.59 (m, 1H), 3.53-3.47 (m, 1H), 2.46 (d, J=0.9 Hz, 3H), 1.50 (s, 9H), 1.16 (t, J=7.2 Hz, 6H).

### Example 178. Synthesis of tert-butyl (S)-4-(7-(3-chlorophenyl)-5-(trifluoromethyl)-7H-pyrrolo[2,3-d]pyrimidin-4-yl)-3-methylpiperazine-1-carboxylate (Compound 494)

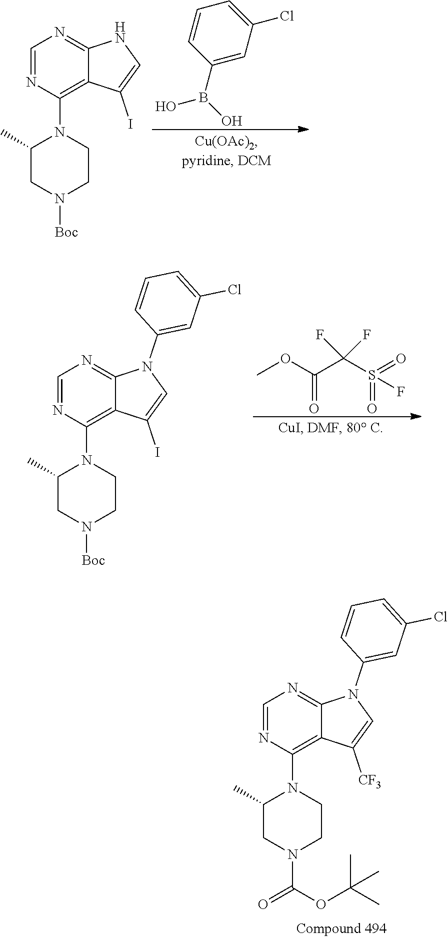

### Step 1. tert-Butyl (S)-4-(7-(3-chlorophenyl)-5-iodo-7H-pyrrolo[2,3-d]pyrimidin-4-yl)-3-methylpiperazine-1-carboxylate

To a solution of tert-butyl (S)-4-(5-iodo-7H-pyrrolo[2,3-d]pyrimidin-4-yl)-3-methylpiperazine-1-carboxylate (300 mg, 0.67 mmol) in DCM (15 mL) were added (3-chlorophenyl)boronic acid (210 mg, 1.4 mmol), 4 A molecular sieves (500 mg), Cu(OAc)₂ (370 mg, 2.0 mmol) and pyridine (0.33 mL, 4.1 mmol). The resulting mixture was stirred at 40° C. under O₂ atmosphere overnight. The reaction was quenched with aqueous NH₄OH (1 mL) at 0° C. and filtered. The filtrate was extracted twice with DCM. The combined organic layers were dried over Na₂SO₄, filtered, concentrated, and the residue was purified by flash column chromatography (silica gel, 0-20%, ethyl acetate in petroleum ether) to afford tert-butyl (S)-4-(7-(3-chlorophenyl)-5-iodo-7H-pyrrolo[2,3-d]pyrimidin-4-yl)-3-methylpiperazine-1-carboxylate (70 mg, 18%) as a white solid. LC/MS ESI (m/z): 554 (M+H)⁺.

### Step 2. tert-Butyl (S)-4-(7-(3-chlorophenyl)-5-(trifluoromethyl)-7H-pyrrolo[2,3-d]pyrimidin-4-yl)-3-methylpiperazine-1-carboxylate

To a solution of tert-butyl (S)-4-(7-(3-chlorophenyl)-5-iodo-7H-pyrrolo[2,3-d]pyrimidin-4-yl)-3-methylpiperazine-1-carboxylate (70 mg, 0.12 mmol) in DMF (5 mL) were added methyl 2,2-difluoro-2-(fluorosulfonyl)acetate (0.06 mL, 0.50 mmol) and CuI (24 mg, 0.12 mmol). The resulting mixture was heated to 80° C. overnight. After cooling to room temperature, the reaction was partitioned between EtOAc and water. The aqueous layer was extracted twice with EtOAc. The combined organic layers were washed with brine, dried over Na₂SO₄, filtered, and concentrated. The residue was purified by flash column chromatography (silica gel, 0˜10%, ethyl acetate in petroleum ether) to give the product which was further purified by prep-HPLC to afford tert-butyl (S)-4-(7-(3-chlorophenyl)-5-(trifluoromethyl)-7H-pyrrolo[2,3-d]pyrimidin-4-yl)-3-methylpiperazine-1-carboxylate (30 mg, 48%) as a white solid. LC/MS ESI (m/z): 496 (M+H)⁺. ¹H NMR (400 MHz, CDCl₃) δ 8.48 (s, 1H), 7.65 (s, 1H), 7.63 (t, J=1.9 Hz, 1H), 7.52-7.49 (m, 1H), 7.44-7.39 (m, 1H), 7.37-7.33 (m, 1H), 4.33-4.27 (m, 1H), 3.99-3.78 (m, 1H), 3.66 (d, J=11.8 Hz, 1H), 3.53-3.34 (m, 3H), 3.20-3.04 (m, 1H), 1.43 (s, 9H), 1.13 (d, J=6.6 Hz, 3H).

The following compound was prepared by a procedure analogous to the synthesis of Compound 494 from the corresponding boric acid.

| Cmpd | | |
|---|---|---|
| No. | Chemical Name | LCMS and ¹H NMR |
| 495 | tert-butyl (S)-4-(7-(3,5- | LC/MS ESI (m/z): 498 (M + H)⁺. ¹H NMR (400 MHz, |
| | difluorophenyl)-5- | CDCl₃) δ 8.55 (s, 1H), 7.72 (s, 1H), 7.37-7.29 (m, 2H), |
| | (trifluoromethyl)-7H- | 6.96-6.81 (m, 1H), 4.45-4.31 (m, 1H), 4.12-3.84 (m, |
| | pyrrolo[2,3-d]pyrimidin-4-yl)-3- | 1H), 3.74 (d, J = 13.3 Hz, 1H), 3.62-3.37 (m, 3H), 3.29- |
| | methylpiperazine-1-carboxylate | 3.02 (m, 1H), 1.50 (s, 9H), 1.21 (d, J = 6.6 Hz, 3H). |

### Example 179. Synthesis of tert-butyl 4-(7-(4-cyanopyridin-2-yl)-5-(pyrrolidin-1-yl)-7H-pyrrolo[2,3-d]pyrimidin-4-yl)-3,3-dimethylpiperazine-1-carboxylate (Compound 601)

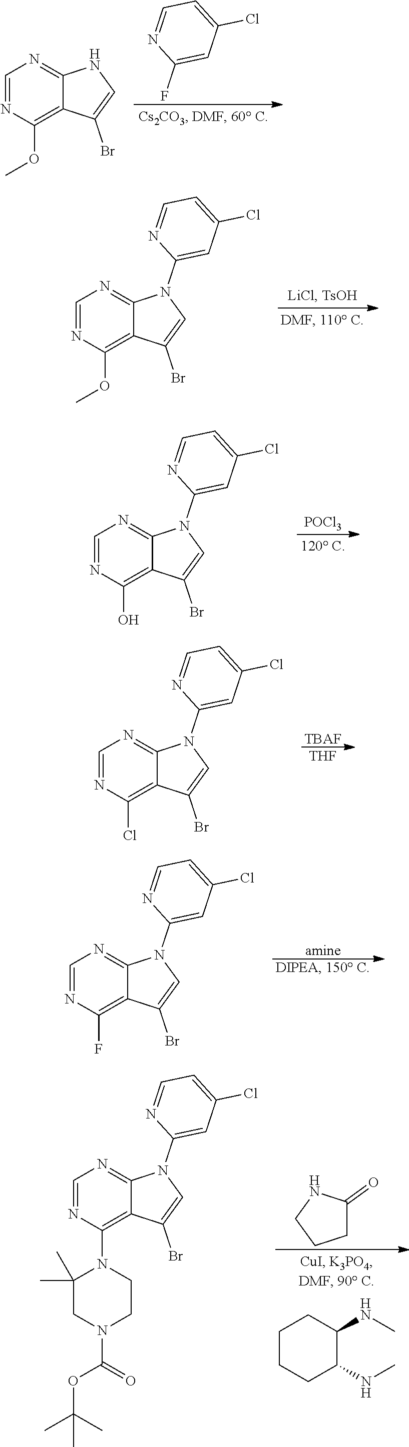

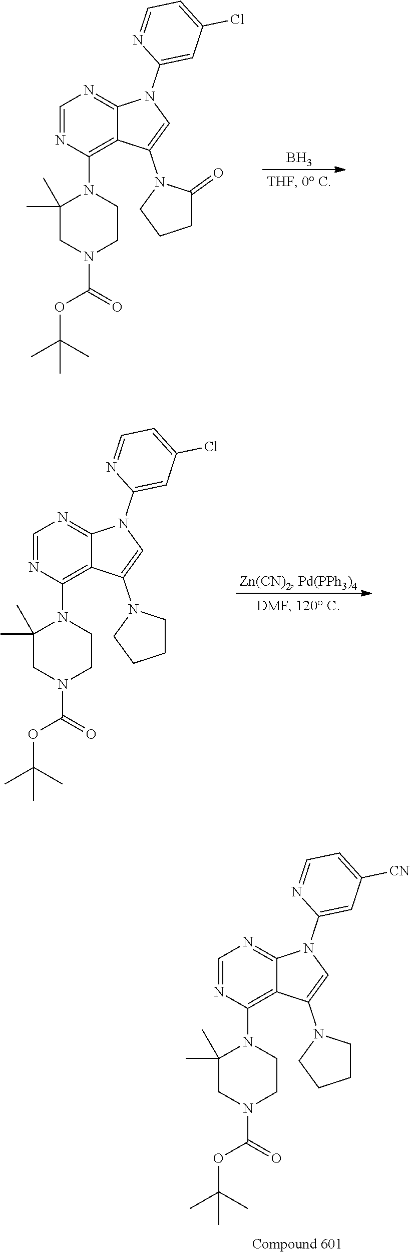

### Step 1. 5-Bromo-7-(4-chloropyridin-2-yl)-4-methoxy-7H-pyrrolo[2,3-d]pyrimidine

To a solution of 5-bromo-4-methoxy-7H-pyrrolo[2,3-d]pyrimidine (3.0 g, 13 mmol) in DMF (15 mL) were added 4-chloro-2-fluoropyridine (3.5 g, 26 mmol) and Cs₂CO₃ (6.5 g, 20 mmol). The resulting mixture was stirred at 60° C. for 12 h. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to afford 5-bromo-7-(4-chloropyridin-2-yl)-4-methoxy-7H-pyrrolo[2,3-d]pyrimidine (2.6 g, 50%) as a yellow solid. LC/MS ESI (m/z): 339, 401 (M+H)⁺.

### Step 2. 5-Bromo-7-(4-chloropyridin-2-yl)-7H-pyrrolo[2,3-d]pyrimidin-4-ol

To a solution of 5-bromo-7-(4-chloropyridin-2-yl)-4-methoxy-7H-pyrrolo[2,3-d]pyrimidine (2.6 g, 6.5 mmol) in DMF (8 mL) were added 4-methylbenzenesulfonic acid (11 g, 65 mmol) and LiCl (2.8 g, 65 mmol). The resulting mixture was stirred at 110° C. for 4 h. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography to afford 5-bromo-7-(4-chloropyridin-2-yl)-7H-pyrrolo[2,3-d]pyrimidin-4-ol (1.7 g, 80%) as a yellow solid. LC/MS ESI (m/z): 325, 327 (M+H)⁺.

### Step 3. 5-Bromo-4-chloro-7-(4-chloropyridin-2-yl)-7H-pyrrolo[2,3-d]pyrimidine

A mixture of 5-bromo-7-(4-chloropyridin-2-yl)-7H-pyrrolo[2,3-d]pyrimidin-4-ol (1.7 g, 5.2 mmol) and POCl₃ (10 mL) was stirred at 120° C. under N₂ overnight. After cooling to room temperature, solvents were removed. The residue was dissolved in DCM and washed with NaHCO₃ (aq.). The organic layer was dried over Na₂SO₄, filtered and concentrated. The residue was purified by flash column chromatography (silica gel, 0-30% ethyl acetate in petroleum ether) to afford 5-bromo-4-chloro-7-(4-chloropyridin-2-yl)-7H-pyrrolo[2,3-d]pyrimidine (1.2 g, 67%) as a light yellow solid. LC/MS ESI (m/z): 343, 345 (M+H)⁺.

### Step 4. 5-Bromo-7-(4-chloropyridin-2-yl)-4-fluoro-7H-pyrrolo[2,3-d]pyrimidine

To a solution of 5-bromo-4-chloro-7-(4-chloropyridin-2-yl)-7H-pyrrolo[2,3-d]pyrimidine (800 mg, 2.3 mmol) in THE (1 mL) at 0° C. was added TBAF (10 ml, 1.0 M in THF). The resulting mixture was stirred room temperature for 1 h. The reaction was quenched with ice water and extracted with EtOAc. The organic layer was dried over Na₂SO₄, filtered and concentrated. The residue was purified by flash column chromatography (silica gel, 0˜30% ethyl acetate in petroleum ether) to afford 5-bromo-7-(4-chloropyridin-2-yl)-4-fluoro-7H-pyrrolo[2,3-d]pyrimidine (500 mg, 66%) as a light yellow solid. LC/MS ESI (m/z): 327, 329 (M+H)⁺.

### Step 5. tert-Butyl 4-(5-bromo-7-(4-cyanopyridin-2-yl)-7H-pyrrolo[2,3-d]pyrimidin-4-yl)-4,7-diazaspiro[2.5]octane-7-carboxylate

To a solution of 5-bromo-7-(4-chloropyridin-2-yl)-4-fluoro-7H-pyrrolo[2,3-d]pyrimidine (230 mg, 0.70 mmol) in DIEA (0.5 mL) was added tert-butyl 3,3-dimethylpiperazine-1-carboxylate (1.5 g, 7.0 mmol). The resulting mixture was stirred at 150° C. under N₂ for 5 h. After cooling to room temperature, the reaction mixture was purified by flash column chromatography (silica gel, 0˜40% ethyl acetate in petroleum ether) to afford tert-butyl 4-(5-bromo-7-(4-chloropyridin-2-yl)-7H-pyrrolo[2,3-d]pyrimidin-4-yl)-3,3-dimethylpiperazine-1-carboxylate (120 mg, 33%) as a yellow solid. LC/MS ESI (m/z): 521, 523 (M+H)⁺.

### Step 6. tert-Butyl 4-(7-(4-chloropyridin-2-yl)-5-(2-oxopyrrolidin-1-yl)-7H-pyrrolo[2,3-d]pyrimidin-4-yl)-3,3-dimethylpiperazine-1-carboxylate

To a solution of tert-butyl 4-(5-bromo-7-(4-chloropyridin-2-yl)-7H-pyrrolo[2,3-d]pyrimidin-4-yl)-3,3-dimethylpiperazine-1-carboxylate (120 mg, 0.23 mmol) in DMF (5 mL) were added pyrrolidin-2-one (29 mg, 0.34 mmol), CuI (44 mg, 0.23 mmol), K₃PO₄ (200 mg, 0.92 mmol) and trans-N¹,N²-dimethylcyclohexane-1,2-diamine (32 mg, 0.23 mmol). The resulting mixture was heated to 90° C. for 2 h. After cooling to room temperature, the reaction mixture was partitioned between EtOAc and water. The aqueous layer was extracted twice with EtOAc. The combined organic layers were dried over Na₂SO₄, filtered and concentrated. The residue was purified by flash column chromatography (silica gel, 0˜70% ethyl acetate in petroleum ether) to give tert-butyl 4-(7-(4-chloropyridin-2-yl)-5-(2-oxopyrrolidin-1-yl)-7H-pyrrolo[2,3-d]pyrimidin-4-yl)-3,3-dimethylpiperazine-1-carboxylate (70 mg, 62%). LC/MS ESI (m/z): 526 (M+H)⁺.

### Step 7. tert-Butyl 4-(7-(4-chloropyridin-2-yl)-5-(pyrrolidin-1-yl)-7H-pyrrolo[2,3-d]pyrimidin-4-yl)-3,3-dimethylpiperazine-1-carboxylate

To a solution of tert-butyl 4-(7-(4-chloropyridin-2-yl)-5-(2-oxopyrrolidin-1-yl)-7H-pyrrolo[2,3-d]pyrimidin-4-yl)-3,3-dimethylpiperazine-1-carboxylate (50 mg, 0.095 mmol) in THF (1 mL) at 0° C. was added BH₃ (2.0 mL, 1.0 M in THF) dropwise. The resulting mixture was stirred at room temperature overnight. The reaction was quenched with saturated NaHCO₃ (aq.) and extracted twice with EtOAc. The combined organic phases were washed with brine, dried over anhydrous Na₂SO₄, filtered and concentrated. The residue was purified by column chromatography on silica gel (0˜50% ethyl acetate in petroleum ether) to give tert-butyl 4-(7-(4-chloropyridin-2-yl)-5-(pyrrolidin-1-yl)-7H-pyrrolo[2,3-d]pyrimidin-4-yl)-3,3-dimethylpiperazine-1-carboxylate (20 mg, 41%) as a yellow solid. LC/MS ESI (m/z): 512 (M+H)⁺.

### Step 8. tert-Butyl 4-(7-(4-cyanopyridin-2-yl)-5-(pyrrolidin-1-yl)-7H-pyrrolo[2,3-d]pyrimidin-4-yl)-3,3-dimethylpiperazine-1-carboxylate

To a solution of tert-butyl 4-(7-(4-chloropyridin-2-yl)-5-(pyrrolidin-1-yl)-7H-pyrrolo[2,3-d]pyrimidin-4-yl)-3,3-dimethylpiperazine-1-carboxylate (20 mg, 0.039 mmol) in DMF (5 mL) were added Zn(CN)₂ (23 mg, 0.20 mmol) and Pd(PPh₃)₄ (45 mg, 0.039 mmol). The resulting mixture was heated to 120° C. overnight. After cooling to room temperature, solvents were removed and the residue was purified by flash column chromatography (silica gel, 0˜60% ethyl acetate in petroleum ether) and prep-HPLC to afford tert-butyl 4-(7-(4-cyanopyridin-2-yl)-5-(pyrrolidin-1-yl)-7H-pyrrolo[2,3-d]pyrimidin-4-yl)-3,3-dimethylpiperazine-1-carboxylate (5.0 mg, 26%) as a yellow solid. LC/MS ESI (m/z): 503 (M+H)⁺. ¹H NMR (400 MHz, CDCl₃) δ 9.28 (s, 1H), 8.49 (d, J=5.0 Hz, 1H), 8.43 (s, 1H), 7.52 (s, 1H), 7.21 (dd, J=5.1, 0.9 Hz, 1H), 3.53 (s, 2H), 3.48 (d, J=5.2 Hz, 2H), 3.29 (s, 2H), 3.09 (s, 4H), 1.91 (s, 4H), 1.53 (s, 6H), 1.43 (s, 9H).

### Example 180. Synthesis of 2-(methyl-d₃)propan-2-yl-1,1,1,3,3,3-d₆ (2R,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate (Compound 602)

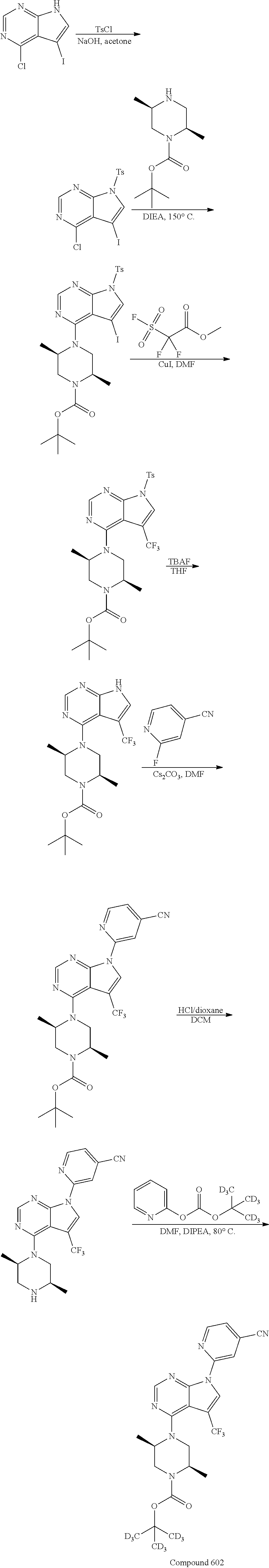

### Step 11. 4-Chloro-5-iodo-7-tosyl-7H-pyrrolo[2,3-d]pyrimidine

To a solution of 4-chloro-5-iodo-7H-pyrrolo[2,3-d]pyrimidine (200 g, 0.71 mol) and 4-methylbenzene-1-sulfonyl chloride (180 g, 0.93 mol) in acetone (2 L) at 0° C. was added dropwise 2.0 M NaOH (0.53 L). The reaction was allowed to warm to room temperature and stirred for 3 hours. The precipitate was collected by filtration, washed twice with water and dried in vacuo to afford 4-chloro-5-iodo-7-tosyl-7H-pyrrolo[2,3-d]pyrimidine (300 g, 95%) as an off white solid. LC/MS ESI (m/z): 434 (M+H)⁺.

### Step 2. tert-Butyl (2R,5R)-4-(5-iodo-7-tosyl-7H-pyrrolo[2,3-d]pyrimidin-4-yl)-2,5-dimethylpiperazine-1-carboxylate

To a solution of 4-chloro-5-iodo-7-tosyl-7H-pyrrolo[2,3-d]pyrimidine (340 mg, 0.77 mmol) in DIPEA (0.41 mL, 2.3 mmol) was added tert-butyl (2R,5R)-2,5-dimethylpiperazine-1-carboxylate (250 mg, 1.2 mmol). The resulting mixture was stirred at 150° C. for 2 h. After cooling to room temperature, the reaction was concentrated and purified by flash column chromatography (silica gel, 0˜30%, ethyl acetate in petroleum ether) to afford tert-butyl (2R,5R)-4-(5-iodo-7-tosyl-7H-pyrrolo[2,3-d]pyrimidin-4-yl)-2,5-dimethylpiperazine-1-carboxylate (260 mg, 54%) as a yellow solid. LC/MS ESI (m/z): 612 (M+H)⁺.

### Step 3. tert-Butyl (2R,5R)-2,5-dimethyl-4-(7-tosyl-5-(trifluoromethyl)-7H-pyrrolo[2,3-d]pyrimidin-4-yl)piperazine-1-carboxylate

To a solution of tert-butyl (2R,5R)-4-(5-iodo-7-tosyl-7H-pyrrolo[2,3-d]pyrimidin-4-yl)-2,5-dimethylpiperazine-1-carboxylate (260 mg, 0.42 mmol) in DMF (5 mL) were added methyl 2,2-difluoro-2-(fluorosulfonyl)acetate (0.16 mL, 1.30 mmol) and CuI (81 mg, 0.42 mmol). The resulting mixture was heated at 80° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to afford tert-butyl (2R,5R)-2,5-dimethyl-4-(7-tosyl-5-(trifluoromethyl)-7H-pyrrolo[2,3-d]pyrimidin-4-yl)piperazine-1-carboxylate (130 mg, 55%) as a yellow oil. LC/MS ESI (m/z): 554 (M+H)⁺.

### Step 4. tert-Butyl (2R,5R)-2,5-dimethyl-4-(5-(trifluoromethyl)-7H-pyrrolo[2,3-d]pyrimidin-4-yl)piperazine-1-carboxylate

To a solution of tert-butyl (2R,5R)-2,5-dimethyl-4-(7-tosyl-5-(trifluoromethyl)-7H-pyrrolo[2,3-d]pyrimidin-4-yl)piperazine-1-carboxylate (130 mg, 0.23 mmol) in THE (2 mL) was added TBAF (1.4 mL, 1.0 M in THF). The resulting mixture was stirred at room temperature for 1 h. The reaction was quenched with water and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford tert-butyl (2R,5R)-2,5-dimethyl-4-(5-(trifluoromethyl)-7H-pyrrolo[2,3-d]pyrimidin-4-yl)piperazine-1-carboxylate (90 mg, 95%) as a yellow oil. LC/MS ESI (m/z): 400 (M+H)⁺.

### Step 5. tert-Butyl (2R,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate

To a solution of tert-butyl (2R,5R)-2,5-dimethyl-4-(5-(trifluoromethyl)-7H-pyrrolo[2,3-d]pyrimidin-4-yl)piperazine-1-carboxylate (90 mg, 0.22 mmol) in DMF (5 mL) were added 2-fluoroisonicotinonitrile (55 mg, 0.45 mmol) and Cs₂CO₃ (220.2 mg, 0.67 mmol). The resulting mixture was heated to 50° C. for 1 h. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜20%, ethyl acetate in petroleum ether) to afford tert-butyl (2R,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate (80 mg, 70%) as a yellow oil. LC/MS ESI (m/z): 502 (M+H)⁺.

### Step 6. 2-(4-((2R,5R)-2,5-Dimethylpiperazin-1-yl)-5-(trifluoromethyl)-7H-pyrrolo[2,3-d]pyrimidin-7-yl)isonicotinonitrile

To a solution of tert-butyl (2R,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate (80 mg, 0.16 mmol) in DCM (2 mL) was added HCl/dioxane (1.0 mL, 4.0M). The resulting mixture was stirred at room temperature for 2 h. After removal of solvent, the residue was diluted with DCM and washed with NaHCO₃ (aq.). The organic layer was extracted twice with DCM. The combined organic layers were washed with brine, dried over Na₂SO₄, filtered and concentrated. The residue was used directly in the next step. LC/MS ESI (m/z): 402 (M+H)⁺.

### Step 7. 2-(Methyl-d₃)propan-2-yl-1,1,1,3,3,3-d₆ (2R,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate

To a solution of 2-(4-((2R,5R)-2,5-dimethylpiperazin-1-yl)-5-(trifluoromethyl)-7H-pyrrolo[2,3-d]pyrimidin-7-yl)isonicotinonitrile (60 mg, 0.15 mmol) in DMF (2 mL) were added 2-(methyl-d₃)propan-2-yl-1,1,1,3,3,3-d₆ pyridin-2-yl carbonate (3.0 eq., prepared following the procedure outlined for compound 625) and DIPEA (1 mL). The resulting mixture was heated to 80° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜20%, ethyl acetate in petroleum ether) to give crude product which was further purified by prep-HPLC to afford 2-(methyl-d₃)propan-2-yl-1,1,1,3,3,3-d₆ (2R,5R)-4-(7-(4-cyanopyridin-2-yl)-5-(trifluoromethyl)-7H-pyrrolo[2,3-d]pyrimidin-4-yl)-2,5-dimethylpiperazine-1-carboxylate (37 mg, 48%) as a white solid. LC/MS ESI (m/z): 511 (M+H)⁺. ¹H NMR (400 MHz, CDCl₃) δ 9.27 (s, 1H), 8.61-8.58 (m, 2H), 8.56 (s, 1H), 7.41 (dd, J=5.0, 1.2 Hz, 1H), 4.44-4.35 (m, 1H), 4.04-3.88 (m, 2H), 3.58 (dd, J=13.2, 5.4 Hz, 1H), 3.27 (dd, J=13.2, 8.2 Hz, 1H), 2.99 (dd, J=13.9, 11.5 Hz, 1H), 1.14 (d, J=6.3 Hz, 3H), 1.02 (d, J=6.2 Hz, 3H).

The following compound was prepared by a procedure analogous to the synthesis of compound 602 from the corresponding chiral amine.

| Cmpd | | |
|---|---|---|
| No. | Chemical Name | LCMS and ¹H NMR |
| 603 | 2-(methyl-d3)propan-2-yl- | LC/MS ESI (m/z): 511 (M + H)⁺. ¹H NMR (400 MHz, |
| | 1,1,1,3,3,3-d6 (2S,5S)-4-(7-(4- | CDCl₃) δ 9.28 (s, 1H), 8.61-8.57 (m, 2H), 8.56 (s, 1H), |
| | cyanopyridin-2-yl)-5- | 7.43-7.40 (m, 1H), 4.43-4.35 (m, 1H), 4.04-3.90 (m, |
| | (trifluoromethyl)-7H- | 2H), 3.61-3.55 (m, 1H), 3.30-3.23 (m, 1H), 3.03-2.95 |
| | pyrrolo[2,3-d]pyrimidin-4-yl)- | (m, 1H), 1.14 (d, J = 6.3 Hz, 3H), 1.02 (d, J = 6.2 Hz, 3H). |
| | 2,5-dimethylpiperazine-1- | |
| | carboxylate | |

### Example 181. Synthesis of 1-(trifluoromethyl)cyclobutyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (Compound 604)

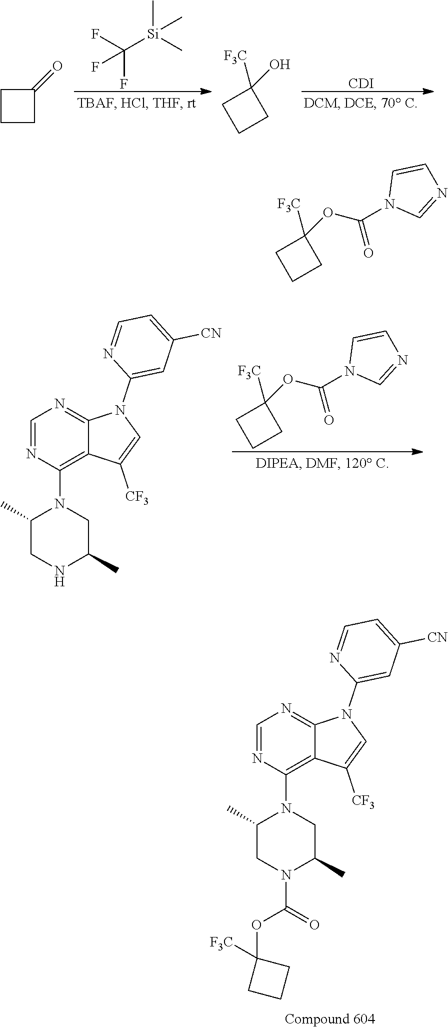

### Step 1. 1-(Trifluoromethyl)cyclobutan-1-ol

To a solution of cyclobutanone (500 mg, 7.1 mmol) in THF (5 mL) were added trimethyl(trifluoromethyl)silane (1200 mg, 8.6 mmol), and TBAF (0.10 mL, 1.0 M in THF). After stirring at room temperature for 2 h, the mixture was treated with 6 M HCl (1.6 mL) and stirred for 1 h. The reaction was dried over Na₂SO₄ and filtered. The filtrate was used directly in the next step.

### Step 2. 1-(Trifluoromethyl)cyclobutyl 1H-imidazole-1-carboxylate

The solution of 1-(trifluoromethyl)cyclobutan-1-ol (filtrate) was treated with of DCE (20 mL) and CDI (1300 mg, 8.0 mmol). The resulting mixture was heated to 70° C. overnight. After cooling to room temperature, solvent was removed and the residue was purified by flash column chromatography (silica gel, 0-30% ethyl acetate in petroleum ether) to afford 1-(trifluoromethyl)cyclobutyl 1H-imidazole-1-carboxylate (200 mg) as a solid. LCMS ESI (m/z): 235 (M+H)⁺.

### Step 3. 1-(Trifluoromethyl)cyclobutyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate

To a solution of 1-(trifluoromethyl)cyclobutyl 1H-imidazole-1-carboxylate (70 mg, 0.30 mmol) in DMF (5 mL) were added 2-(4-((2S,5R)-2,5-dimethylpiperazin-1-yl)-5-(trifluoromethyl)-7H-pyrrolo[2,3-d]pyrimidin-7-yl)isonicotinonitrile (40 mg, 0.10 mmol, prepared following a similar procedure outlined for compound 602) and DIPEA (200 mg, 1.6 mmol). The resulting mixture was heated to 120° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜35% ethyl acetate in petroleum ether) to give crude product, which was further purified by prep-HPLC to afford 1-(trifluoromethyl)cyclobutyl (2R,5S)-4-(7-(4-cyanopyridin-2-yl)-5-(trifluoromethyl)-7H-pyrrolo[2,3-d]pyrimidin-4-yl)-2,5-dimethylpiperazine-1-carboxylate (15 mg, 27%) as a white solid. LC/MS ESI (m/z): 568 (M+H)⁺. ¹H NMR (400 MHz, CDCl₃) δ 9.27 (s, 1H), 8.63-8.57 (m, 2H), 8.52 (s, 1H), 7.42 (dd, J=5.0, 1.2 Hz, 1H), 4.61-4.20 (m, 2H), 3.76-3.58 (m, 3H), 3.47-3.37 (m, 1H), 2.87-2.71 (m, 2H), 2.57-2.44 (m, 2H), 1.97-1.91 (m, 1H), 1.86-1.77 (m, 1H), 1.09 (s, 6H).

### Example 182. Synthesis of 1-methylcyclobutyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (Compound 605)

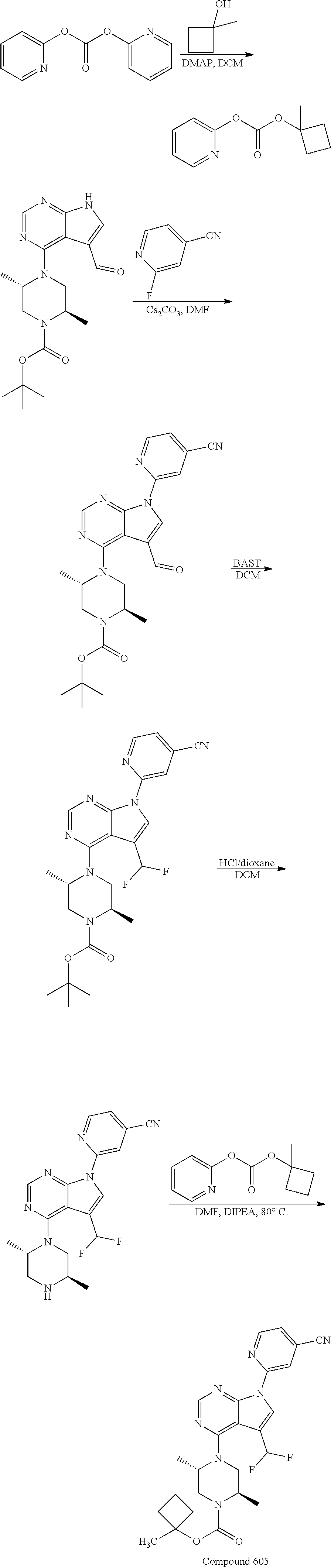

### Step 11. tert-Butyl (2R,5S)-4-(7-(4-cyanopyridin-2-yl)-5-formyl-7H-pyrrolo[2,3-d]pyrimidin-4-yl)-2,5-dimethylpiperazine-1-carboxylate

To a solution of tert-butyl (2R,5S)-4-(5-formyl-7H-pyrrolo[2,3-d]pyrimidin-4-yl)-2,5-dimethylpiperazine-1-carboxylate (760 mg, 2.1 mmol, prepared following the procedure outlined for compound 635) in DMF (10 mL) were added 2-fluoroisonicotinonitrile (520 mg, 4.2 mmol) and Cs₂CO₃ (2100 mg, 6.3 mmol). The resulting mixture was stirred at 50° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0-30%, ethyl acetate in petroleum ether) to afford tert-butyl (2R,5S)-4-(7-(4-cyanopyridin-2-yl)-5-formyl-7H-pyrrolo[2,3-d]pyrimidin-4-yl)-2,5-dimethylpiperazine-1-carboxylate (720 mg, 73%) as a yellow solid. LC/MS ESI (m/z): 462 (M+H)⁺.

### Step 2. tert-Butyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate

To a solution of tert-butyl (2R,5S)-4-(7-(4-cyanopyridin-2-yl)-5-formyl-7H-pyrrolo[2,3-d]pyrimidin-4-yl)-2,5-dimethylpiperazine-1-carboxylate (720 mg, 1.6 mmol) in DCM (5 mL) at 0° C. was added BAST (2.8 mL, 16 mmol) dropwise. After stirring at room temperature overnight, the reaction was quenched with NaHCO₃ (aq.). The aqueous layer was extracted twice with DCM. The combined organic layers were washed with brine, dried over Na₂SO₄, concentrated and purified by flash column chromatography (silica gel, 0˜20%, ethyl acetate in petroleum ether) to afford tert-butyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (440 mg, 58%) as a white solid. LC/MS ESI (m/z): 484 (M+H)⁺.

### Step 3. 2-(5-(Difluoromethyl)-4-((2S,5R)-2,5-dimethylpiperazin-1-yl)-7H-pyrrolo[2,3-d]pyrimidin-7-yl)isonicotinonitrile

To a solution of tert-butyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (280 mg, 0.57 mmol) in DCM (10 mL) at 0° C. was added HCl/dioxane (5.0 mL, 4.0M). After stirring at room temperature for 2 h, the reaction was quenched with NaHCO₃ (aq.). The aqueous layer was extracted twice with DCM. The combined organic layers were washed with brine, dried over Na₂SO₄, concentrated, and purified by flash column chromatography (silica gel, 0˜10%, methanol in dichloromethane) to afford 2-(5-(difluoromethyl)-4-((2S,5R)-2,5-dimethylpiperazin-1-yl)-7H-pyrrolo[2,3-d]pyrimidin-7-yl)isonicotinonitrile (150 mg, 67%) as a yellow solid. LC/MS ESI (m/z): 384 (M+H)⁺.

### Step 4. 1-Methylcyclobutyl pyridin-2-yl carbonate

To a solution of 1-methylcyclobutan-1-ol (100 mg, 1.2 mmol) in DCM (5 mL) were added di(pyridin-2-yl) carbonate (250 mg, 1.2 mmol) and DMAP (43 mg, 0.34 mmol). After stirring at room temperature overnight, the solvents were removed and the residue was used directly in the next step.

### Step 5. 1-Methylcyclobutyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate

To a solution of 2-(5-(difluoromethyl)-4-((2S,5R)-2,5-dimethylpiperazin-1-yl)-7H-pyrrolo[2,3-d]pyrimidin-7-yl)isonicotinonitrile (45 mg, 0.11 mmol) in DMF (2 mL) were added 1-methylcyclobutyl pyridin-2-yl carbonate (1.0 mL, 0.23 mmol) and DIPEA (0.50 mL, 0.11 mmol). The resulting mixture was stirred at 80° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by prep-HPLC to afford 1-methylcyclobutyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (17 mg, 29%) as a white solid. LC/MS ESI (m/z): 496 (M+H)⁺. ¹H NMR (400 MHz, CDCl₃) δ 9.27 (s, 1H), 8.58 (d, J=4.9 Hz, 1H), 8.50 (s, 1H), 8.45 (s, 1H), 7.38 (d, J=4.9 Hz, 1H), 6.89 (t, J=55.6 Hz, 1H), 4.56-4.47 (m, 1H), 4.41-4.25 (m, 1H), 3.76-3.66 (m, 2H), 3.56 (m, 1H), 3.46 (d, J=13.1 Hz, 1H), 2.27 (m, 2H), 2.15-2.04 (m, 2H), 1.80-1.70 (m, 1H), 1.64-1.55 (m, 1H), 1.52 (s, 3H), 1.12 (d, J=6.6 Hz, 3H), 1.07 (d, J=6.8 Hz, 3H).

The following compounds were prepared by procedures similar to the synthesis of compound 605 from the corresponding chiral amine and alcohol (Aryl groups of compound 607 and compound 608 were introduced via Ullmann coupling conditions).

| Cmpd | | |
|---|---|---|
| No. | Chemical Name | LCMS and ¹H NMR |
| 606 | 2-(methyl-d₃)propan-2-yl- | LC/MS ESI (m/z): 493 (M + H)⁺. ¹H NMR (400 MHz, |
| | 1,1,1,3,3,3-d₆ (2S,5R)-4-(7-(4- | CDCl₃) δ 9.34 (s, 1H), 8.65 (dd, J = 5.0, 0.6 Hz, 1H), 8.57 |
| | cyanopyridin-2-yl)-5- | (s, 1H), 8.52 (d, J = 2.0 Hz, 1H), 7.45 (dd, J = 5.0, 1.4 Hz, |
| | (difluoromethyl)-7H- | 1H), 6.96 (t, J = 55.6 Hz, 1H), 4.62-4.55 (m, 1H), 4.40 (s, |
| | pyrrolo[2,3-d]pyrimidin-4-yl)- | 1H), 3.82-3.75 (m, 2H), 3.61 (dd, J = 13.4, 3.5 Hz, 1H), |
| | 2,5-dimethylpiperazine-1- | 3.53 (d, J = 13.2 Hz, 1H), 1.19 (d, J = 6.6 Hz, 3H), 1.13 (d, |
| | carboxylate | J = 6.8 Hz, 3H). |
| 607 | 1-methylcyclopropyl (2R,5S)-4- | LC/MS ESI (m/z): 492 (M + H)⁺. ¹H NMR (400 MHz, |
| | (5-(difluoromethyl)-7-(3,5- | CD₃OD) δ 8.42 (s, 1H), 8.01 (s, 1H), 7.61-7.52 (m, 2H), |
| | difluorophenyl)-7H-pyrrolo[2,3- | 7.31-7.01 (m, 2H), 4.69-4.61 (m, 1H), 4.45-4.23 (m, |
| | d]pyrimidin-4-yl)-2,5- | 1H), 3.85-3.71 (m, 2H), 3.68-3.57 (m, 2H), 1.55 (s, 3H), |
| | dimethylpiperazine-1- | 1.16-1.10 (m, 6H), 0.89 (t, J = 5.4 Hz, 2H), 0.67 (t, J = 6.1 |
| | carboxylate | Hz, 2H). |
| 608 | 1-methylcyclopropyl (2R,5S)-4- | LC/MS ESI (m/z): 490 (M + H)⁺. ¹H NMR (400 MHz, |
| | (7-(3-chlorophenyl)-5- | CDCl₃) δ 8.44 (s, 1H), 7.67-7.61 (m, 1H), 7.56-7.50 (m, |
| | (difluoromethyl)-7H- | 2H), 7.44-7.38 (m, 1H), 7.35-7.31 (m, 1H), 6.90 (t, J = |
| | pyrrolo[2,3-d]pyrimidin-4-yl)- | 55.9 Hz, 1H), 4.54-4.21 (m, 2H), 3.77-3.62 (m, 2H), |
| | 2,5-dimethylpiperazine-1- | 3.59-3.51 (m, 1H), 3.49-3.42 (m, 1H), 1.51 (s, 3H), 1.12- |
| | carboxylate | 1.06 (m, 6H), 0.85-0.80 (m, 2H), 0.65-0.55 (m, 2H). |
| 609 | tert-buty1 4-(7-(4-cyanopyridin- | LC/MS ESI (m/z): 456(M + H)⁺. ¹H NMR (400 MHz, |
| | 2-yl)-5-(difluoromethyl)-7H- | CDCl3) δ 9.33 (s, 1H), 8.66 (d, J = 4.9 Hz, 1H), 8.60 (s, |
| | pyrrolo[2,3-d]pyrimidin-4- | 1H), 8.55 (t, J = 1.9 Hz, 1H), 7.46 (dd, J = 5.0, 1.2 Hz, 1H), |
| | yl)piperazine-1-carboxylate | 6.99 (t, J = 55.4 Hz, 1H), 3.73-3.52 (m, 8H), 1.50 (s, 9H). |
| 610 | tert-butyl (2R,5S)-4-(7-(4- | LC/MS ESI (m/z): 487 (M + H)⁺. ¹H NMR (400 MHz, |
| | cyanopyridin-2-yl)-5- | CD3OD) δ 9.21 (s, 1H), 8.72 (d, J = 5.0 Hz, 1H), 8.53 (s, |
| | (difluoromethyl)-7H- | 2H), 7.64 (d, J = 5.0 Hz, 1H), 7.18 (t, J = 56 Hz, 1H), 4.68- |
| | pyrrolo[2,3-d]pyrimidin-4-yl)-5- | 4.59 (m, 1H), 4.35 (s, 1H), 3.82 (dd, J = 13.4, 4.0 Hz, |
| | methyl-2-(methyl-d3)piperazine- | 1H), 3.76 (dd, J = 13.5, 2.4 Hz, 1H), 3.68-3.53 (m, 2H), |
| | 1-carboxylate | 1.50 (s, 9H), 1.17 (d, J = 6.6 Hz, 3H). |
| 611 | 1-methylcyclopropyl (2R,5S)-4- | LC/MS ESI (m/z): 475 (M + H)⁺. ¹H NMR (400 MHz, |
| | (5-(difluoromethyl)-7-(4- | CDCl3) δ 8.75 (dd, J = 10.7, 2.2 Hz, 1H), 8.56 (s, 1H), 8.53 |
| | fluoropyridin-2-yl)-7H- | (t, J = 2.1 Hz, 1H), 8.48-8.43 (m, 1H), 7.00 (m, 1H), 6.95 |
| | pyrrolo[2,3-d]pyrimidin-4-yl)- | (t, J = 56 Hz, 1H), 4.59-4.51 (m, 1H), 4.50-4.06 (m, |
| | 2,5-dimethylpiperazine-1- | 1H), 3.80-3.70 (m, 2H), 3.62 (dd, J = 13.4, 3.6 Hz, 1H), |
| | carboxylate | 3.51 (d, J = 12 Hz, 1H), 1.58 (s, 3H), 1.14 (m, 6H), 0.93- |
| | | 0.86 (m, 2H), 0.70-0.62 (m, 2H). |

### Example 183. Synthesis of tert-butyl (S)-4-(1-(4-cyanopyridin-2-yl)-3-(1-methylcyclopropyl)-1H-pyrrolo[3,2-c]pyridin-4-yl)-3-methylpiperazine-1-carboxylate (Compound 612)

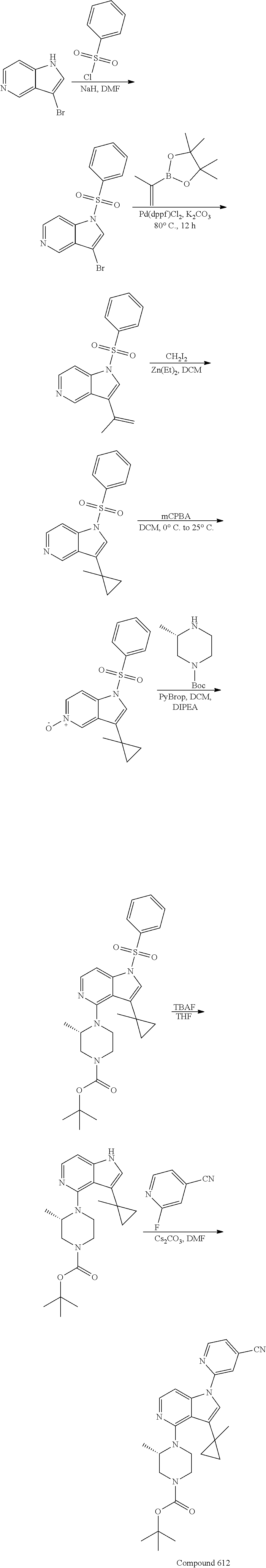

### Step 1. 3-Bromo-1-(phenylsulfonyl)-1H-pyrrolo[3,2-c]pyridine

To a suspension of NaH (610 mg, 15 mmol, 60 wt %) in anhydrous DMF (10 mL) at 0° C. was added 3-bromo-1H-pyrrolo[3,2-c]pyridine (2.0 g, 10 mmol) in portions, followed by benzenesulfonyl chloride (2.0 g, 11 mmol) dropwise. After stirring at room temperature for 2 h, the reaction was poured into ice water and extracted twice with EtOAc. The combined organic layers were washed with brine, dried over Na₂SO₄, filtered and concentrated. The residue was purified by flash column chromatography (silica gel, 0-40% ethyl acetate in petroleum ether) to afford 3-bromo-1-(phenylsulfonyl)-1H-pyrrolo[3,2-c]pyridine (3.0 g, 89%) as a solid. LC/MS ESI (m/z): 337, 339 (M+H)⁺.

### Step 2. 1-(Phenylsulfonyl)-3-(prop-1-en-2-yl)-1H-pyrrolo[3,2-c]pyridine

To a solution of 3-bromo-1-(phenylsulfonyl)-1H-pyrrolo[3,2-c]pyridine (3.0 g, 8.9 mmol) in dioxane (30 mL) and water (1 mL) were added 4,4,5,5-tetramethyl-2-(prop-1-en-2-yl)-1,3,2-dioxaborolane (2.2 g, 13 mmol), K₂CO₃ (3.7 g, 27 mmol) and Pd(dppf)Cl₂ (650 mg, 0.80 mmol), the resulting mixture was heated to 80° C. overnight. After cooling to room temperature, solvents were removed and the residue was purified by flash column chromatography (silica gel, 0-40% ethyl acetate in petroleum ether) to afford 1-(phenylsulfonyl)-3-(prop-1-en-2-yl)-1H-pyrrolo[3,2-c]pyridine (2.0 g, 76%) as a solid. LC/MS ESI (m/z): 299 (M+H)⁺.

### Step 3. 1-(Phenylsulfonyl)-3-(prop-1-en-2-yl)-1H-pyrrolo[3,2-c]pyridine

To a solution of Et₂Zn (17 mL, 1.0 M in toluene) in DCM (8 mL) at 0° C. was added dropwise a solution of CH₂I₂ (0.72 mL, 8.5 mmol) in DCM (2 mL). After 20 min, a solution of 1-(phenylsulfonyl)-3-(prop-1-en-2-yl)-1H-pyrrolo[3,2-c]pyridine (500 mg, 1.7 mmol) in DCM (5 mL) was added dropwise. The resulting mixture was stirred at room temperature overnight. The reaction was quenched with NH₄Cl (aq.) and extracted twice with DCM. The combined organic layers were washed with brine, dried over Na₂SO₄, filtered and concentrated. The residue was purified by flash column chromatography (silica gel, 0-30%, ethyl acetate in petroleum ether) to afford 3-(1-methylcyclopropyl)-1-(phenylsulfonyl)-1H-pyrrolo[3,2-c]pyridine (310 mg, 60%) as a solid. LC/MS ESI (m/z): 313 (M+H)⁺.

### Step 4. 3-(1-Methylcyclopropyl)-1-(phenylsulfonyl)-1H-pyrrolo[3,2-c]pyridine 5-oxide

To a solution of 3-(1-methylcyclopropyl)-1-(phenylsulfonyl)-1H-pyrrolo[3,2-c]pyridine (1.2 g, 3.9 mmol) in DCM (15 m1) at 0° C. was added 3-chlorobenzoperoxoic acid (1.3 g, 7.7 mmol) in portions. The resulting mixture was stirred at room temperature under N₂ overnight. The reaction was diluted with H₂O and extracted twice with DCM. The combined organic layers were washed with brine, dried over Na₂SO₄, filtered and concentrated. The residue was purified by flash column chromatography (silica gel, 0˜15% MeOH in DCM) to afford 3-(1-methylcyclopropyl)-1-(phenylsulfonyl)-1H-pyrrolo[3,2-c]pyridine 5-oxide (950 mg, 75%) as a yellow solid. LC/MS ESI (m/z): 329 (M+H)⁺.

### Step 5. tert-Butyl (S)-3-methyl-4-(3-(1-methylcyclopropyl)-1-(phenylsulfonyl)-1H-pyrrolo[3,2-c]pyridin-4-yl)piperazine-1-carboxylate

To a solution of 3-(1-methylcyclopropyl)-1-(phenylsulfonyl)-1H-pyrrolo[3,2-c]pyridine 5-oxide (330 mg, 1.0 mmol, tert-butyl (S)-3-methylpiperazine-1-carboxylate (1.0 g, 5.0 mmol) and DIPEA (480 mg, 3.8 mmol) in DCM (10 mL) was added PyBroP (610 mg, 1.3 mmol) in portions. The resulting mixture was stirred at room temperature under N₂ overnight. The reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (S)-3-methyl-4-(3-(1-methylcyclopropyl)-1-(phenylsulfonyl)-1H-pyrrolo[3,2-c]pyridin-4-yl)piperazine-1-carboxylate (120 mg, 24% yield) as a withe solid. LC/MS ESI (m/z): 511 (M+H)⁺.

### Step 6. tert-Butyl (S)-3-methyl-4-(3-(1-methylcyclopropyl)-1-(phenylsulfonyl)-1H-pyrrolo[3,2-c]pyridin-4-yl)piperazine-1-carboxylate

To a solution of tert-butyl (S)-3-methyl-4-(3-(1-methylcyclopropyl)-1-(phenylsulfonyl)-1H-pyrrolo[3,2-c]pyridin-4-yl)piperazine-1-carboxylate (120 mg, 0.24 mmol) in THE (1 mL) was added TBAF (2.4 mL, 1.0 M in THF). After stirring at room temperature under N₂ for 2 h, the reaction was partitioned between EtOAc and brine. The aqueous layer was extracted twice with EtOAc. The combined organic layers were washed with brine, dried over Na₂SO₄, filtered and concentrated to afford crude tert-butyl (S)-3-methyl-4-(3-(1-methylcyclopropyl)-1H-pyrrolo[3,2-c]pyridin-4-yl)piperazine-1-carboxylate (340 mg that contained TBAF-related impurities) as light yellow oil. LC/MS ESI (m/z): 371 (M+H)⁺.

### Step 7. tert-Butyl (S)-4-(1-(4-cyanopyridin-2-yl)-3-(1-methylcyclopropyl)-1H-pyrrolo[3,2-c]pyridin-4-yl)-3-methylpiperazine-1-carboxylate

To a solution of tert-butyl (S)-3-methyl-4-(3-(1-methylcyclopropyl)-1H-pyrrolo[3,2-c]pyridin-4-yl)piperazine-1-carboxylate (340 mg that contained TBAF-related impurities, ˜0.20 mmol) and 2-fluoroisonicotinonitrile (120 mg, 1.0 mmol) in DMF (1 mL) was added Cs₂CO₃ (330 mg, 1.0 mmol). After stirring at room temperature under N₂ overnight, the reaction was partitioned between EtOAc and NH₄Cl (aq.). The aqueous layer was extracted twice with EtOAc. The combined organic layers were washed with brine, dried over Na₂SO₄, filtered and concentrated. The residue was purified by flash column chromatography (silica gel, 0˜40% ethyl acetate in petroleum ether) and prep-HPLC to afford tert-butyl (S)-4-(1-(4-cyanopyridin-2-yl)-3-(1-methylcyclopropyl)-1H-pyrrolo[3,2-c]pyridin-4-yl)-3-methylpiperazine-1-carboxylate (62 mg, 66% yield) as a white solid. LC/MS ESI (m/z): 473. (M+H)⁺. ¹H NMR (400 MHz, CDCl₃) δ 8.72 (d, J=5.0 Hz, 1H), 8.22 (d, J=5.8 Hz, 1H), 7.89 (d, J=5.8 Hz, 1H), 7.64 (s, 1H), 7.42 (s, 1H), 7.39 (d, J=5.0 Hz, 1H), 3.88 (s, 2H), 3.77 (s, 1H), 3.54 (t, J=9.8 Hz, 1H), 3.41 (s, 1H), 3.17 (dd, J=12.2, 8.1 Hz, 1H), 3.03 (t, J=9.0 Hz, 1H), 1.65 (s, 3H), 1.50 (s, 9H), 1.17-1.09 (m, 1H), 0.99 (d, J=6.1 Hz, 3H), 0.90-0.84 (m, 1H), 0.75 (m, 2H).

### Example 184. Synthesis of 1-methylcyclopropyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (Compound 613)

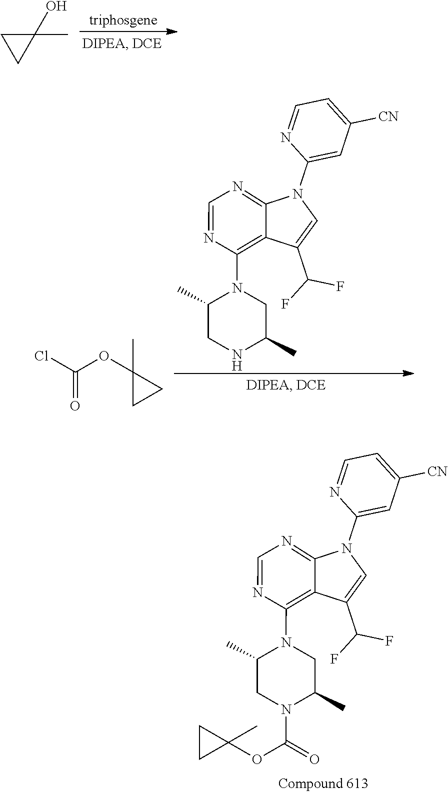

To a solution of 1-methylcyclopropan-1-ol (100 mg, 1.4 mmol) and DIPEA (0.10 mL) in DCE (2 mL) at 0° C. was added triphosgene (70 mg, 0.23 mmol). After stirring at room temperature for 30 min, 2-(5-(difluoromethyl)-4-((2S,5R)-2,5-dimethylpiperazin-1-yl)-7H-pyrrolo[2,3-d]pyrimidin-7-yl)isonicotinonitrile (30 mg, 0.078 mmol, prepared following the procedure outlined for compound 605) was added. After 3 h, the reaction was partitioned between DCM and water. The aqueous layer was extracted twice with DCM. The combined organic layers were washed with brine, dried over Na₂SO₄, filtered and concentrated. The residue was purified by flash column chromatography (silica gel, 0-30%, ethyl acetate in petroleum ether) to give crude product which was further purified by prep-HPLC to afford 1-methylcyclopropyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (12 mg, 15%) as a white solid. LC/MS ESI (m/z): 482 (M+H)⁺. ¹H NMR (400 MHz, CDCl₃) δ 9.27 (s, 1H), 8.58 (d, J=5.0 Hz, 1H), 8.50 (s, 1H), 8.45 (s, 1H), 7.38 (d, J=5.0 Hz, 1H), 6.88 (t, J=55.6 Hz, 1H), 4.57-4.46 (m, 1H), 4.44-4.14 (m, 1H), 3.76-3.62 (m, 2H), 3.55 (dd, J=13.4, 3.6 Hz, 1H), 3.45 (d, J=13.2 Hz, 1H), 1.51 (s, 3H), 1.10 (d, J=6.6 Hz, 3H), 1.05 (d, J=6.8 Hz, 3H), 0.85-0.80 (m, 2H), 0.62-0.56 (m, 2H).

### Example 185. Synthesis of 1-(trifluoromethyl)cyclobutyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (Compound 614)

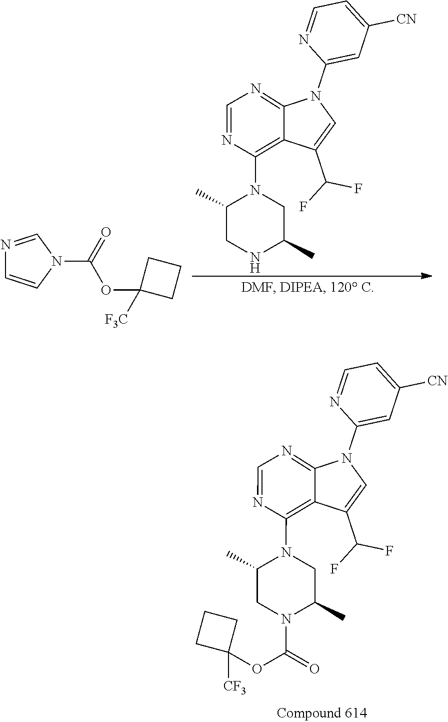 [SMILES: O=C(OC1(C(F)(F)F)CCC1)n1ccnc1 ; C[C@@H]1CN(c2ncnc3c2c(C(F)F)cn3-c2cc(C#N)ccn2)[C@@H](C)CN1 ; C[C@@H]1CN(c2ncnc3c2c(C(F)F)cn3-c2cc(C#N)ccn2)[C@@H](C)CN1C(=O)OC1(C(F)(F)F)CCC1]

To a solution of 2-(5-(difluoromethyl)-4-((2S,5R)-2,5-dimethylpiperazin-1-yl)-7H-pyrrolo[2,3-d]pyrimidin-7-yl)isonicotinonitrile (20 mg, 0.052 mmol, prepared following the procedure outlined for compound 605) in DMF (2 mL) were added 1-(trifluoromethyl)cyclobutyl 1H-imidazole-1-carboxylate (18 mg, 0.078 mmol, prepared following the procedure outlined for compound 604) and DIPEA (0.10 mL, 0.052 mmol). The resulting mixture was stirred at 120° C. overnight. After cooling to room temperature, the reaction was partitioned between EtOAc and water. The aqueous layer was extracted twice with EtOAc twice. The combined organic layers were washed with brine, dried over Na₂SO₄, filtered and concentrated. The residue was purified by flash column chromatography (silica gel, 0-30% ethyl acetate in petroleum ether) to give crude product which was further purified by prep-HPLC to afford 1-(trifluoromethyl)cyclobutyl (2R,5S)-4-(7-(4-cyanopyridin-2-yl)-5-(difluoromethyl)-7H-pyrrolo[2,3-d]pyrimidin-4-yl)-2,5-dimethylpiperazine-1-carboxylate (10 mg, 36%) as a white solid. LC/MS ESI (m/z): 550 (M+H)⁺. ¹H NMR (400 MHz, CDCl₃) δ 9.27 (s, 1H), 8.59 (d, J=5.0 Hz, 1H), 8.51 (s, 1H), 8.45 (s, 1H), 7.39 (d, J=5.0 Hz, 1H), 6.88 (t, J=55.6 Hz, 1H), 4.60-4.45 (m, 1H), 4.44-4.20 (m, 1H), 3.78-3.57 (m, 3H), 3.48 (d, J=13.4 Hz, 1H), 2.88-2.69 (m, 2H), 2.56-2.40 (m, 2H), 2.01-1.87 (m, 1H), 1.87-1.76 (m, 1H), 1.13-1.08 (m, 6H).

### Example 186. Synthesis of tert-butyl (3S)-4-(7-(4-cyanopyridin-2-yl)-5-(2-methylcyclopropyl)-7H-pyrrolo[2,3-d]pyrimidin-4-yl)-3-methylpiperazine-1-carboxylate (Compound 615)

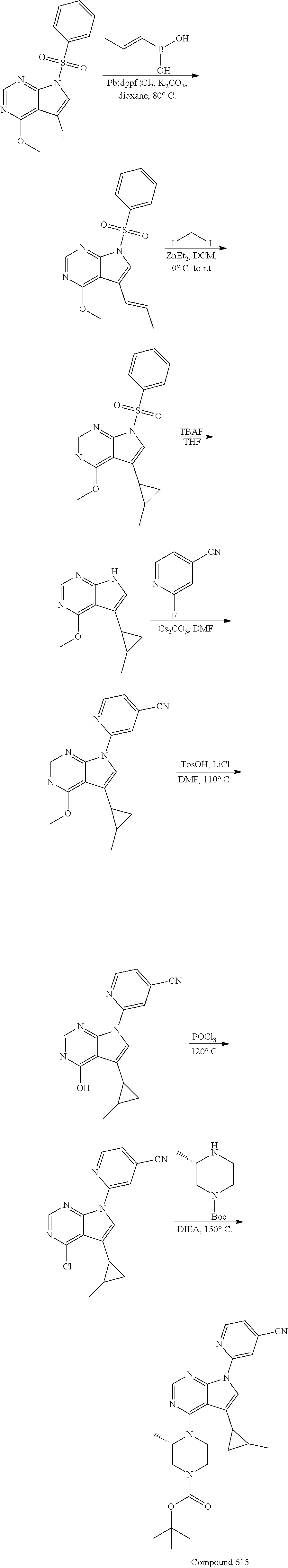

### Step 1. (E)-4-Methoxy-7-(phenylsulfonyl)-5-(prop-1-en-1-yl)-7H-pyrrolo[2,3-d]pyrimidine

To a solution of 5-iodo-4-methoxy-7-(phenylsulfonyl)-7H-pyrrolo[2,3-d]pyrimidine (690 mg, 1.8 mmol) in dioxane (10 mL) and H₂O (1 mL) were added (E)-prop-1-en-1-ylboronic acid (200 mg, 2.3 mmol), K₂CO₃ (990 mg, 7.2 mmol) and Pd(dppf)Cl₂ (130 mg, 0.18 mmol). The resulting mixture was heated to 80° C. overnight. After cooling to room temperature, solvents were removed and the residue was purified by flash column chromatography (silica gel, 0-20%, ethyl acetate in petroleum ether) to afford (E)-4-methoxy-7-(phenylsulfonyl)-5-(prop-1-en-1-yl)-7H-pyrrolo[2,3-d]pyrimidine (300 mg, 49%) as a yellow solid. LC/MS ESI (m/z): 330 (M+H)⁺.

### Step 2. 4-Methoxy-5-(2-methylcyclopropyl)-7-(phenylsulfonyl)-7H-pyrrolo[2,3-d]pyrimidine

To a solution of Et₂Zn (9.2 mL, 1.0 M in toluene) in DCM (2 mL) at 0° C. was added CH₂I₂ (1.2 g, 4.6 mmol) dropwise. After 30 min, a solution of (E)-4-methoxy-7-(phenylsulfonyl)-5-(prop-1-en-1-yl)-7H-pyrrolo[2,3-d]pyrimidine (300 mg, 0.91 mmol) in DCM (10 mL) was added dropwise. The resulting mixture was stirred 0° C. for 1 h and then warmed to room temperature overnight. The reaction was quenched with NH₄Cl (aq.) and extracted twice with DCM. The combined organic layers were washed with brine, dried over Na₂SO₄, filtered and concentrated. The residue was purified by flash column chromatography (silica gel, 0˜20% ethyl acetate in petroleum ether) to afford 4-methoxy-5-(2-methylcyclopropyl)-7-(phenylsulfonyl)-7H-pyrrolo[2,3-d]pyrimidine (150 mg, 48%) as a yellow solid. LC/MS ESI (m/z): 344 (M+H)⁺.

### Step 3. 4-Methoxy-5-(2-methylcyclopropyl)-7H-pyrrolo[2,3-d]pyrimidine

To a solution of 4-methoxy-5-(2-methylcyclopropyl)-7-(phenylsulfonyl)-7H-pyrrolo[2,3-d]pyrimidine (150 mg, 0.44 mmol) in THE (3 mL) was added TBAF (3.9 mL, 1.0 M in THF). After stirring at 50° C. for 1 h, the reaction was diluted with water and extracted twice with EtOAc. The combined organic layers were washed with brine, dried over Na₂SO₄, filtered and concentrated. The residue was purified by flash column chromatography (silica gel, 0˜60%, ethyl acetate in petroleum ether) to give 4-methoxy-5-(2-methylcyclopropyl)-7H-pyrrolo[2,3-d]pyrimidine (80 mg, 90%) as a yellow solid. LC/MS ESI (m/z): 204 (M+H)⁺.

### Step 4. 2-(4-Methoxy-5-(2-methylcyclopropyl)-7H-pyrrolo[2,3-d]pyrimidin-7-yl)isonicotinonitrile

To a solution of 4-methoxy-5-(2-methylcyclopropyl)-7H-pyrrolo[2,3-d]pyrimidine (40 mg, 0.20 mmol) in DMF (10 mL) were added 2-fluoroisonicotinonitrile (49 mg, 0.40 mmol) and Cs₂CO₃ (650 mg, 2.0 mmol). After stirring at 50° C. for 1 h, the reaction was quenched with ice water and extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to give 2-(4-methoxy-5-(2-methylcyclopropyl)-7H-pyrrolo[2,3-d]pyrimidin-7-yl)isonicotinonitrile (50 mg, 82%) as a yellow solid. LC/MS ESI (m/z): 306 (M+H)⁺.

### Step 5. 2-(4-Hydroxy-5-(2-methylcyclopropyl)-7H-pyrrolo[2,3-d]pyrimidin-7-yl)isonicotinonitrile

To a solution of 2-(4-methoxy-5-(2-methylcyclopropyl)-7H-pyrrolo[2,3-d]pyrimidin-7-yl)isonicotinonitrile (50 mg, 0.16 mmol) in DMF (2 mL) were added p-toluenesulfonic acid (280 mg, 1.6 mmol) and LiCl (67 mg, 1.6 mmol). The resulting mixture was heated to 110° C. for 2 h. After cooling to room temperature, the reaction was diluted with water and extracted twice with EtOAc. The combined organic layers were washed with brine, dried over Na₂SO₄, filtered, and concentrated to afford 2-(4-hydroxy-5-(2-methylcyclopropyl)-7H-pyrrolo[2,3-d]pyrimidin-7-yl)isonicotinonitrile (40 mg, 85%) as a yellow solid. LC/MS ESI (m/z): 292 (M+H)⁺.

### Step 6. 2-(4-Chloro-5-(2-methylcyclopropyl)-7H-pyrrolo[2,3-d]pyrimidin-7-yl)isonicotinonitrile

A mixture of 2-(4-hydroxy-5-(2-methylcyclopropyl)-7H-pyrrolo[2,3-d]pyrimidin-7-yl)isonicotinonitrile (40 mg, 0.14 mmol) and POCl₃ (5 mL) was heated to 120° C. for 2 h. After cooling to room temperature, solvent was removed. The residue was dissolved in DCM, washed with NaHCO₃ (aq.), dried over Na₂SO₄, filtered and concentrated. The residue was used directly in the next step. LC/MS ESI (m/z): 310 (M+H)⁺.

### Step 7. tert-Butyl (3S)-4-(7-(4-cyanopyridin-2-yl)-5-(2-methylcyclopropyl)-7H-pyrrolo[2,3-d]pyrimidin-4-yl)-3-methylpiperazine-1-carboxylate

To a solution of 2-(4-chloro-5-(2-methylcyclopropyl)-7H-pyrrolo[2,3-d]pyrimidin-7-yl)isonicotinonitrile (25 mg, 0.080 mmol) in DIPEA (1 mL) was added tert-butyl (S)-3-methylpiperazine-1-carboxylate (320 mg, 1.6 mmol). The resulting mixture was heated to 150° C. for 6 h. After cooling to room temperature, solvent was removed and the residue was purified by flash column chromatography (silica gel, 0˜20%, ethyl acetate in petroleum ether) to give crude product which was further purified by prep-HPLC to afford tert-butyl (3S)-4-(7-(4-cyanopyridin-2-yl)-5-(2-methylcyclopropyl)-7H-pyrrolo[2,3-d]pyrimidin-4-yl)-3-methylpiperazine-1-carboxylate (3.6 mg, 9.0%) as a white solid. LC/MS ESI (m/z): 474 (M+H)⁺. ¹H NMR (400 MHz, CDCl₃) δ 9.25 (s, 1H), 8.52 (dd, J=8.7, 5.0 Hz, 1H), 8.43 (s, 1H), 7.89-7.62 (m, 1H), 7.28 (t, J=6.0 Hz, 1H), 4.85-4.56 (m, 1H), 4.14-3.87 (m, 2H), 3.82-3.72 (m, 1H), 3.54-3.29 (m, 2H), 3.14-2.97 (m, 1H), 2.00-1.90 (m, 1H), 1.69-1.56 (m, 2H), 1.43 (s, 9H), 1.42-1.39 (m, 1H), 1.15-1.09 (m, 3H), 0.82-0.69 (m, 3H).

The following compound was prepared by procedures analogous to the synthesis of compound 615 using the corresponding 3-iodobenzonitrile using standard Ullmann coupling conditions.

| Cmpd | | |
|---|---|---|
| No. | Chemical Name | LCMS and ¹H NMR |
| 616 | tert-butyl (3S)-4-(7-(3- | LC/MS ESI (m/z): 473 (M + H)⁺. ¹H NMR (400 MHz, |
| | cyanophenyl)-5-(2- | CDCl₃) δ 8.44 (s, 1H), 8.05-7.97 (m, 2H), 7.63-7.58 (m, |
| | methylcyclopropyl)-7H- | 2H), 7.09-6.85 (m, 1H), 4.94-4.68 (m, 1H), 4.20-3.95 |
| | pyrrolo[2,3-d]pyrimidin-4-yl)-3- | (m, 2H), 3.90-3.79 (m, 1H), 3.62-3.37 (m, 2H), 3.22- |
| | methylpiperazine-1-carboxylate | 3.03 (m, 1H), 1.80-1.71 (m, 1H), 1.68-1.61 (m, 2H), |
| | | 1.51 (s, 9H), 1.44-1.42 (m, 1H), 1.26 (d, J = 4.9 Hz, 3H), |
| | | 0.91-0.77 (m, 3H). |

### Example 187. Synthesis of tert-butyl (2R,5S)-4-(7-(4-cyanopyrimidin-2-yl)-5-(2,2,2-trifluoroethyl)-7H-pyrrolo[2,3-d]pyrimidin-4-yl)-2,5-dimethylpiperazine-1-carboxylate (Compound 617)

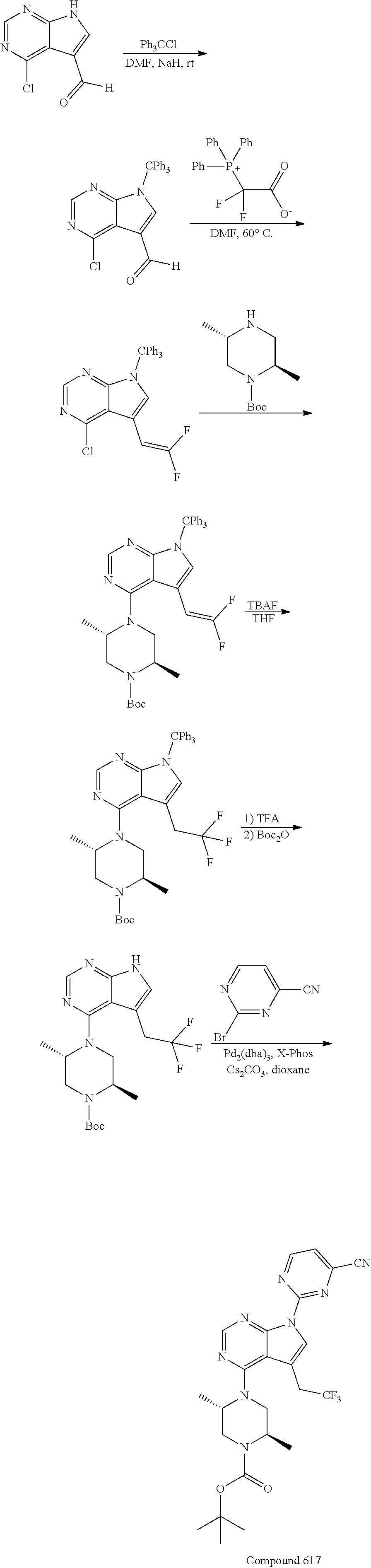

### Step 1. 4-Chloro-7-trityl-7H-pyrrolo[2,3-d]pyrimidine-5-carbaldehyde

To a suspension of NaH (1.2 g, 30 mmol, 60 wt %) in anhydrous DMF (15 mL) at 0° C. was added 4-chloro-7H-pyrrolo[2,3-d]pyrimidine-5-carbaldehyde (1.1 g, 6.1 mmol) in portions, followed by trityl chloride (3.4 g, 12 mmol) in portions. The resulting mixture was stirred at room temperature for 3 h. The reaction was poured into ice water and extracted twice with EtOAc. The combined organic layers were washed with brine, dried over Na₂SO₄, filtered and concentrated. The residue was purified by flash column chromatography (silica gel, 0-30%, ethyl acetate in petroleum ether) to afford 4-chloro-7-trityl-7H-pyrrolo[2,3-d]pyrimidine-5-carbaldehyde (1.9 g, 74%) as a light yellow solid. LC/MS ESI (m/z): 424 (M+H)⁺.

### Step 2. 4-Chloro-5-(2,2-difluorovinyl)-7-trityl-7H-pyrrolo[2,3-d]pyrimidine

To a solution of 4-chloro-7-trityl-7H-pyrrolo[2,3-d]pyrimidine-5-carbaldehyde (1.1 g, 2.6 mmol) in DMF (15 m1) was added 2,2-difluoro-2-(triphenylphosphonio)acetate (1.9 g, 5.2 mmol). The resulting mixture was stirred at 60° C. under N₂ for 4 h. The reaction was quenched with H₂O and extracted twice with EtOAc. The combined organic layers were washed with brine, dried over Na₂SO₄, filtered and concentrated. The residue was purified by flash column chromatography (silica gel, 0-50%, ethyl acetate in petroleum ether) to afford 4-chloro-5-(2,2-difluorovinyl)-7-trityl-7H-pyrrolo[2,3-d]pyrimidine (800 mg, 67%) as a yellow solid. LC/MS ESI (m/z): 458 (M+H)⁺.

### Step 3. tert-Butyl (2R,5S)-4-(5-(2,2-difluorovinyl)-7-trityl-7H-pyrrolo[2,3-d]pyrimidin-4-yl)-2,5-dimethylpiperazine-1-carboxylate

A mixture of 4-chloro-5-(2,2-difluorovinyl)-7-trityl-7H-pyrrolo[2,3-d]pyrimidine (800 mg, 1.8 mmol) and tert-butyl (2R,5S)-2,5-dimethylpiperazine-1-carboxylate (1.9 g, 8.7 mmol) was stirred at 150° C. for 3 h. After cooling to room temperature, the reaction was purified by flash column chromatography (silica gel, 0˜50%, ethyl acetate in petroleum ether) to afford tert-butyl (2R,5S)-4-(5-(2,2-difluorovinyl)-7-trityl-7H-pyrrolo[2,3-d]pyrimidin-4-yl)-2,5-dimethylpiperazine-1-carboxylate (310 mg, 28%) as a white solid. LC/MS ESI (m/z): 636 (M+H)⁺.

### Step 4. tert-Butyl (2R,5S)-2,5-dimethyl-4-(5-(2,2,2-trifluoroethyl)-7-trityl-7H-pyrrolo[2,3-d]pyrimidin-4-yl)piperazine-1-carboxylate

To a solution of tert-butyl (2R,5S)-4-(5-(2,2-difluorovinyl)-7-trityl-7H-pyrrolo[2,3-d]pyrimidin-4-yl)-2,5-dimethylpiperazine-1-carboxylate (310 mg, 0.49 mmol) in THE (2 mL) was added TBAF (5.0 mL, 1.0M in THF). After stirring at room temperature overnight, the reaction was quenched with water and extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-2,5-dimethyl-4-(5-(2,2,2-trifluoroethyl)-7-trityl-7H-pyrrolo[2,3-d]pyrimidin-4-yl)piperazine-1-carboxylate (210 mg, 66%) as a yellow solid. LC/MS ESI (m/z): 656 (M+H)⁺.

### Step 5. tert-Butyl (2R,5S)-2,5-dimethyl-4-(5-(2,2,2-trifluoroethyl)-7H-pyrrolo[2,3-d]pyrimidin-4-yl)piperazine-1-carboxylate

To a solution of tert-butyl (2R,5S)-2,5-dimethyl-4-(5-(2,2,2-trifluoroethyl)-7-trityl-7H-pyrrolo[2,3-d]pyrimidin-4-yl)piperazine-1-carboxylate (210 mg, 0.32 mmol) in DCM (5 mL) was added TFA (1.0 m1). The resulting mixture was stirred at room temperature for 3 h. The reaction was quenched with NaHCO₃ (aq.) and extracted twice with DCM. The combined organic layers were dried over Na₂SO₄, filtered and concentrated to afford 4-((2S,5R)-2,5-dimethylpiperazin-1-yl)-5-(2,2,2-trifluoroethyl)-7H-pyrrolo[2,3-d]pyrimidine (85 mg, 85%) as a light yellow solid. LC/MS ESI (m/z): 314 (M+H)⁺.

To a solution of 4-((2S,5R)-2,5-dimethylpiperazin-1-yl)-5-(2,2,2-trifluoroethyl)-7H-pyrrolo[2,3-d]pyrimidine (85 mg, 0.27 mmol) in DCM (5 m1) were added di-tert-butyl dicarbonate (120 mg, 0.54 mmol) and TEA (82 mg, 0.81 mmol). After stirring at room temperature for 3 h, the reaction was quenched with water and extracted twice with DCM. The combined organic layers were washed with brine, dried over Na₂SO₄, filtered and concentrated. The residue was purified by flash column chromatography (silica gel, 0˜70%, ethyl acetate in petroleum ether) to afford tert-butyl (2R,5S)-2,5-dimethyl-4-(5-(2,2,2-trifluoroethyl)-7H-pyrrolo[2,3-d]pyrimidin-4-yl)piperazine-1-carboxylate (80 mg, 71%) as a white solid. LC/MS ESI (m/z): 414 (M+H)⁺.

### Step 6. tert-Butyl (2R,5S)-4-(7-(4-cyanopyrimidin-2-yl)-5-(2,2,2-trifluoroethyl)-7H-pyrrolo[2,3-d]pyrimidin-4-yl)-2,5-dimethylpiperazine-1-carboxylate

To a solution of tert-butyl (2R,5S)-2,5-dimethyl-4-(5-(2,2,2-trifluoroethyl)-7H-pyrrolo[2,3-d]pyrimidin-4-yl)piperazine-1-carboxylate (40 mg, 0.10 mmol) in dioxane (10 mL) were added 2-bromopyrimidine-4-carbonitrile (36 mg, 0.19 mmol), Pd₂(dba)₃ (44 mg, 0.05 mmol), X-Phos (56 mg, 0.09 mmol) and Cs₂CO₃ (95 mg, 0.29 mmol). The resulting mixture was stirred at 100° C. overnight. After cooling to room temperature, the reaction was partitioned between EtOAc and water. The aqueous layer was extracted twice with EtOAc. The combined organic layers were dried over Na₂SO₄, filtered and concentrated. The residue was purified by flash chromatography (silica gel, 0˜50% ethyl acetate in petroleum ether) to give crude product which was further purified by prep-HPLC to afford tert-butyl (2R,5S)-4-(7-(4-cyanopyrimidin-2-yl)-5-(2,2,2-trifluoroethyl)-7H-pyrrolo[2,3-d]pyrimidin-4-yl)-2,5-dimethylpiperazine-1-carboxylate (5.0 mg, 11%) as a white solid. LC/MS ESI (m/z): 517 (M+H)⁺. ¹H NMR (400 MHz, CDCl₃) δ 9.08 (d, J=4.8 Hz, 1H), 8.63 (s, 1H), 8.05 (s, 1H), 7.47 (d, J=4.8 Hz, 1H), 4.35-4.23 (m, 2H), 3.81-3.69 (m, 2H), 3.64-3.54 (m, 3H), 3.28-3.22 (m, 1H), 1.42 (s, 9H), 1.08 (d, J=6.6 Hz, 3H), 1.04 (d, J=6.8 Hz, 3H).

### Example 188. Synthesis of tert-butyl (2R,5S)-4-(7-(6-cyanopyrimidin-4-yl)-5-(2,2,2-trifluoroethyl)-7H-pyrrolo[2,3-d]pyrimidin-4-yl)-2,5-dimethylpiperazine-1-carboxylate (Compound 618)

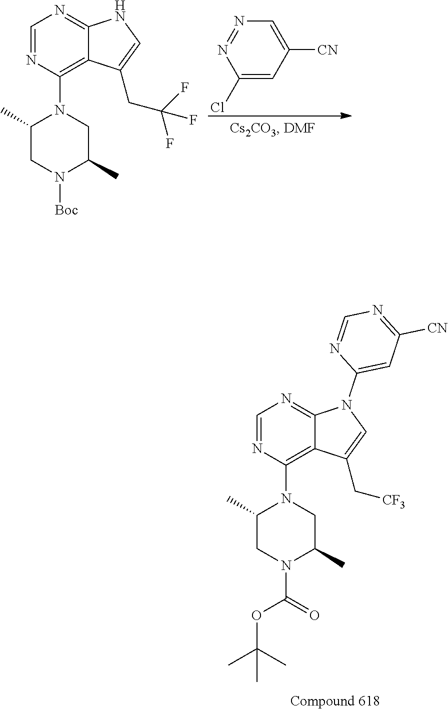

To a solution of tert-butyl (2R,5S)-2,5-dimethyl-4-(5-(2,2,2-trifluoroethyl)-7H-pyrrolo[2,3-d]pyrimidin-4-yl)piperazine-1-carboxylate (40 mg, 0.10 mmol, prepared following the procedure outlined for compound 617) in DMF (5 mL) were added 6-chloropyrimidine-4-carbonitrile (27 mg, 0.19 mmol) and Cs₂CO₃ (95 mg, 0.29 mmol). After stirring at room temperature overnight, the reaction was quenched with H₂O (50 mL) and extracted twice with EtOAc. The combined organic layers were dried over Na₂SO₄, filtered and concentrated. The residue was purified by flash column chromatography (silica gel, 0-50% ethyl acetate in petroleum ether) to give crude product which was further purified by prep-HPLC to afford tert-butyl (2R,5S)-4-(7-(6-cyanopyrimidin-4-yl)-5-(2,2,2-trifluoroethyl)-7H-pyrrolo[2,3-d]pyrimidin-4-yl)-2,5-dimethylpiperazine-1-carboxylate (16 mg, 30%) as a white solid. LC/MS ESI (m/z): 517 (M+H)⁺. ¹H NMR (400 MHz, CDCl₃) δ 9.50 (s, 1H), 9.05 (s, 1H), 8.52 (s, 1H), 8.20 (s, 1H), 4.36-4.27 (m, 2H), 3.79-3.69 (m, 2H), 3.63-3.53 (m, 3H), 3.30-3.25 (m, 1H), 1.43 (s, 9H), 1.10 (d, J=6.6 Hz, 3H), 1.03 (d, J=6.8 Hz, 3H).

The following compound was prepared by a procedure analogous to the synthesis of compound 618 from the corresponding aryl halide.

| Cmpd | | |
|---|---|---|
| No. | Chemical Name | LCMS and ¹H NMR |
| 619 | tert-butyl (2R,5S)-4-(7-(4- | LC/MS ESI (m/z): 516 (M + H)⁺. ¹H NMR (400 MHz, |
| | cyanopyridin-2-yl)-5-(2,2,2- | CDCl₃) δ 9.28 (s, 1H), 8.56 (d, J = 4.9 Hz, 1H), 8.50 (s, |
| | trifluoroethyl)-7H-pyrrolo[2,3- | 1H), 8.18 (s, 1H), 7.34 (d, J = 4.9 Hz, 1H), 4.28 (s, 2H), |
| | d]pyrimidin-4-yl)-2,5- | 3.79-3.69 (m, 2H), 3.65-3.54 (m, 3H), 3.27 (d, J = 12.8 |
| | dimethylpiperazine-1- | Hz, 1H), 1.43 (s, 9H), 1.06 (t, J = 7.3 Hz, 6H). |
| | carboxylate | |

### Example 189. Synthesis of tert-butyl (2R,5S)-4-(1-(4-cyanopyridin-2-yl)-3-(trifluoromethyl)-1H-pyrrolo[3,2-c]pyridin-4-yl)-2,5-dimethylpiperazine-1-carboxylate (Compound 620)

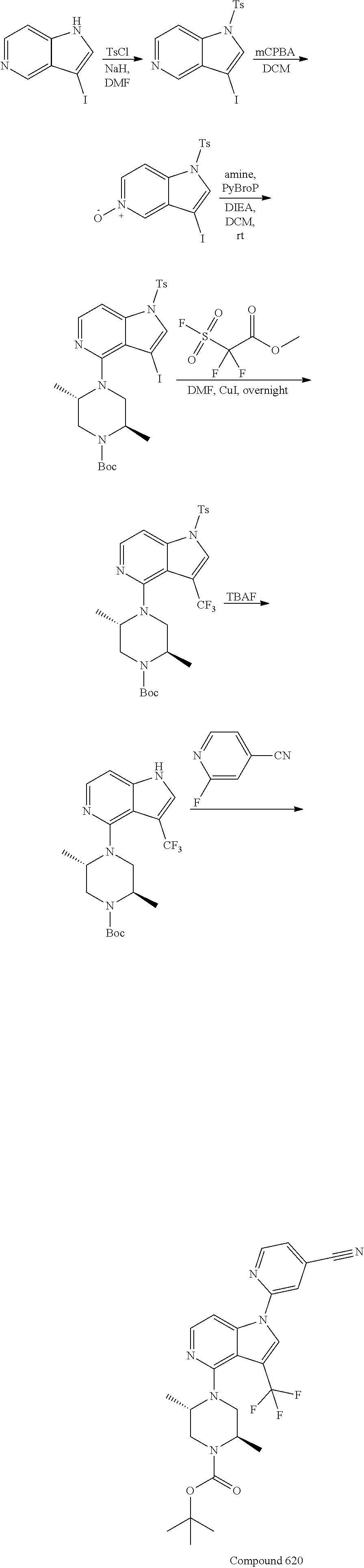

### Step 1. 3-Iodo-1-tosyl-1H-pyrrolo[3,2-c]pyridine

To a suspension of NaH (740 mg, 18 mmol, 60 wt %) in anhydrous DMF (10 mL) at 0° C. was added 3-iodo-1H-pyrrolo[3,2-c]pyridine (1.5 g, 6.2 mmol) in portions, followed by 4-methylbenzenesulfonyl chloride (1.8 g, 9.2 mmol) in portions. After 10 min, the reaction was poured into ice water and extracted twice with EtOAc. The combined organic layers were washed with brine, dried over Na₂SO₄, filtered and concentrated. The residue was purified by flash column chromatography (silica gel, 0˜30% ethyl acetate in petroleum ether) to afford 3-iodo-1-tosyl-1H-pyrrolo[3,2-c]pyridine (2.2 g, 89%) as a light yellow solid. LC/MS ESI (m/z): 399 (M+H)⁺.

### Step 2. 3-Iodo-1-tosyl-1H-pyrrolo[3,2-c]pyridine 5-oxide

To a solution of 3-iodo-1-tosyl-1H-pyrrolo[3,2-c]pyridine (2.2 g, 5.5 mmol) in DCM (15 m1) at 0° C. was added 3-chloroperoxybenzoic acid (1.9 g, 11 mmol) in portions. After stirring at room temperature under N₂ overnight, the reaction was quenched with H₂O and extracted twice with DCM. The combined organic layers were washed with brine, dried over Na₂SO₄, filtered and concentrated. The residue was purified by flash column chromatography (silica gel, 0˜15% MeOH in DCM) to afford 3-iodo-1-tosyl-1H-pyrrolo[3,2-c]pyridine 5-oxide (1.8 g, 78%) as a yellow solid. LC/MS ESI (m/z): 415 (M+H)⁺.

### Step 3. tert-Butyl (2R,5S)-4-(3-iodo-1-tosyl-1H-pyrrolo[3,2-c]pyridin-4-yl)-2,5-dimethylpiperazine-1-carboxylate

To a solution of 3-iodo-1-tosyl-1H-pyrrolo[3,2-c]pyridine 5-oxide (1.1 g, 2.7 mmol) in DCM (10 mL) were added tert-butyl (2R,5S)-2,5-dimethylpiperazine-1-carboxylate (2.9 g, 13 mmol), PyBroP (1.6 g, 3.5 mmol), DIPEA (1.4 g, 11 mmol). After stirring at room temperature overnight, the reaction was quenched with water and extracted twice with DCM. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-4-(3-iodo-1-tosyl-1H-pyrrolo[3,2-c]pyridin-4-yl)-2,5-dimethylpiperazine-1-carboxylate (600 mg, 40%) as a white solid. LC/MS ESI (m/z): 611 (M+H)⁺.

### Step 4. tert-Butyl (2R,5S)-2,5-dimethyl-4-(1-tosyl-3-(trifluoromethyl)-1H-pyrrolo[3,2-c]pyridin-4-yl)piperazine-1-carboxylate

To a solution of tert-butyl (2R,5S)-4-(3-iodo-1-tosyl-1H-pyrrolo[3,2-c]pyridin-4-yl)-2,5-dimethylpiperazine-1-carboxylate (400 mg, 0.66 mmol) in DMF (10 mL) were added methyl 2,2-difluoro-2-(fluorosulfonyl)acetate (380 mg, 2.0 mmol) and CuI (130 mg, 0.66 mmol). The resulting mixture was stirred at 80° C. overnight. After cooling to room temperature, the reaction was quenched with water and extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-2,5-dimethyl-4-(1-tosyl-3-(trifluoromethyl)-1H-pyrrolo[3,2-c]pyridin-4-yl)piperazine-1-carboxylate (310 mg, 86%) as a yellow solid. LC/MS ESI (m/z): 553 (M+H)⁺.

### Step 5. tert-Butyl (2R,5S)-2,5-dimethyl-4-(3-(trifluoromethyl)-1H-pyrrolo[3,2-c]pyridin-4-yl)piperazine-1-carboxylate

To a solution of tert-butyl (2R,5S)-2,5-dimethyl-4-(1-tosyl-3-(trifluoromethyl)-1H-pyrrolo[3,2-c]pyridin-4-yl)piperazine-1-carboxylate (310 mg, 0.56 mmol) in THE (10 mL) was added TBAF (10 mL, 1.0 M in THF). After stirring at room temperature overnight, the reaction was quenched with H₂O (10 mL) and extracted twice with EtOAc. The combined organic layers were dried over Na₂SO₄, filtered and concentrated, the residue was purified by flash chromatography (silica gel, 0˜70% ethyl acetate in petroleum ether) to afford tert-butyl (2R,5S)-2,5-dimethyl-4-(3-(trifluoromethyl)-1H-pyrrolo[3,2-c]pyridin-4-yl)piperazine-1-carboxylate (200 mg, 89%) as a light yellow solid. LC/MS ESI (m/z): 399 (M+H)⁺.

### Step 6. tert-Butyl (2R,5S)-4-(1-(4-cyanopyridin-2-yl)-3-(trifluoromethyl)-1H-pyrrolo[3,2-c]pyridin-4-yl)-2,5-dimethylpiperazine-1-carboxylate

To a solution of tert-butyl (2R,5S)-2,5-dimethyl-4-(3-(trifluoromethyl)-1H-pyrrolo[3,2-c]pyridin-4-yl)piperazine-1-carboxylate (100 mg, 0.25 mmol) in DMF (8 mL) were added 2-fluoroisonicotinonitrile (92 mg, 0.76 mmol) and Cs₂CO₃ (410 mg, 1.3 mmol). The resulting mixture was heated to 50° C. overnight. After cooling to room temperature, the reaction was quenched with H₂O (50 mL) and extracted twice with EtOAc. The combined organic layers were dried over Na₂SO₄, filtered and concentrated. The residue was purified by flash column chromatography (silica gel, 0˜50% ethyl acetate in petroleum ether) to give crude product which was further purified by prep-HPLC to afford tert-butyl (2R,5S)-4-(1-(4-cyanopyridin-2-yl)-3-(trifluoromethyl)-1H-pyrrolo[3,2-c]pyridin-4-yl)-2,5-dimethylpiperazine-1-carboxylate (35 mg, 28%) as a white solid. LC/MS ESI (m/z): 501 (M+H)⁺. ¹H NMR (400 MHz, CDCl₃) δ 8.74 (d, J=5.0 Hz, 1H), 8.16 (d, J=5.9 Hz, 1H), 8.03 (s, 1H), 7.69 (s, 1H), 7.60 (d, J=5.9 Hz, 1H), 7.49 (dd, J=5.0, 1.1 Hz, 1H), 4.42-4.34 (m, 1H), 3.93-3.87 (m, 1H), 3.74-3.69 (m, 1H), 3.67-3.58 (m, 2H), 2.96-2.91 (m, 1H), 1.42 (s, 9H), 1.22 (d, J=6.8 Hz, 3H), 0.87 (d, J=6.6 Hz, 3H).

### Example 190. Synthesis of 2-(methyl-d3)propan-2-yl-1,1,1,3,3,3-d6 (2R,5S)-4-(1-(4-cyanopyridin-2-yl)-3-(trifluoromethyl)-1H-pyrrolo[3,2-c]pyridin-4-yl)-2,5-dimethylpiperazine-1-carboxylate (Compound 623)

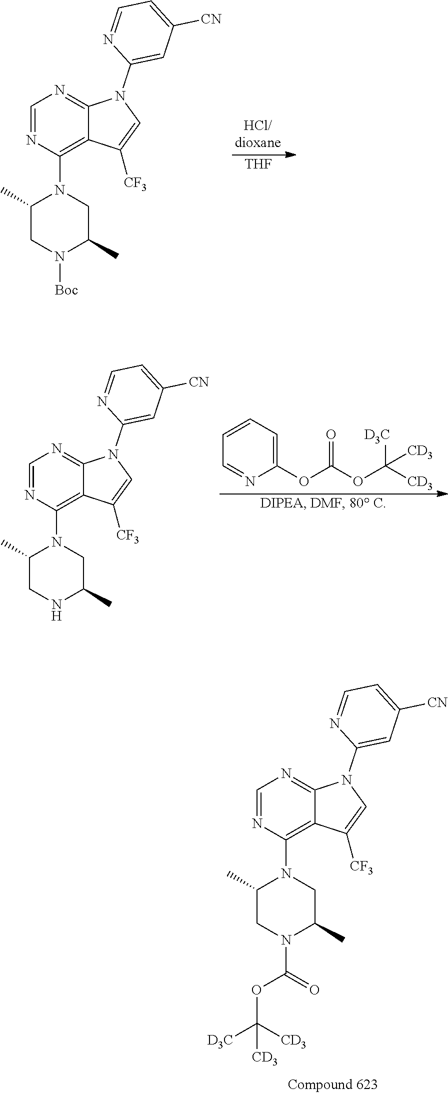

### Step 1. 2-(4-((2S,5R)-2,5-Dimethylpiperazin-1-yl)-3-(trifluoromethyl)-1H-pyrrolo[3,2-c]pyridin-1-yl)isonicotinonitrile

To a solution of tert-butyl (2R,5S)-4-(1-(4-cyanopyridin-2-yl)-3-(trifluoromethyl)-1H-pyrrolo[3,2-c]pyridin-4-yl)-2,5-dimethylpiperazine-1-carboxylate (100 mg, 0.20 mmol, prepared following the procedure outlined for compound 620) in THE (5 mL) was added HCl in dioxane (5.0 ml, 4.0M). After stirring at room temperature overnight, the reaction was quenched with NaHCO₃ (aq.) and extracted twice with EtOAc. The combined organic layers were washed with brine, dried over Na₂SO₄, filtered and concentrated. The residue was purified by flash column chromatography (silica gel, 0-80% ethyl acetate in petroleum ether) to afford 2-(4-((2S,5R)-2,5-dimethylpiperazin-1-yl)-3-(trifluoromethyl)-1H-pyrrolo[3,2-c]pyridin-1-yl)isonicotinonitrile (60 mg, 75%) as a light yellow solid. LC/MS ESI (m/z): 401 (M+H)⁺.

### Step 2. 2-(Methyl-d3)propan-2-yl-1,1,1,3,3,3-d6 (2R,5S)-4-(1-(4-cyanopyridin-2-yl)-3-(trifluoromethyl)-1H-pyrrolo[3,2-c]pyridin-4-yl)-2,5-dimethylpiperazine-1-carboxylate

To a solution of 2-(methyl-d₃)propan-1,1,1,3,3,3-d₆-2-ol (3.0 g, 36 mmol) in DCM (20 mL) were added DMAP (1.3 g, 11 mmol) and di(pyridin-2-yl) carbonate (7.8 g, 36 mmol), the reaction was stirred at 35° C. for 7 days and the resulting reaction solution was used to the next step directly. To a solution of 2-(4-((2S,5R)-2,5-dimethylpiperazin-1-yl)-3-(trifluoromethyl)-1H-pyrrolo[3,2-c]pyridin-1-yl)isonicotinonitrile (60 mg, 0.15 mmol) in DMF (8 mL) was added DIPEA (58 mg, 0.45 mmol), followed by 2-(methyl-d₃)propan-2-yl-1,1,1,3,3,3-d₆ pyridin-2-yl carbonate (0.5 mL of the solution described above). After stirring at 80° C. overnight, DCM was removed, the residue diluted with water and extracted twice with EtOAc. The combined organic layers were washed with brine, dried over Na₂SO₄, filtered and concentrated. The residue was purified by flash column chromatography (silica gel, 0˜5% ethyl acetate in petroleum ether) to give crude product which was further purified by prep-HPLC to afford 2-(methyl-d₃)propan-2-yl-1,1,1,3,3,3-d₆ (2R,5S)-4-(1-(4-cyanopyridin-2-yl)-3-(trifluoromethyl)-1H-pyrrolo[3,2-c]pyridin-4-yl)-2,5-dimethylpiperazine-1-carboxylate (55 mg, 72% yield) as a white solid. LC/MS ESI (m/z): 510 (M+H)⁺. ¹H NMR (400 MHz, CDCl₃) δ 8.74 (dd, J=5.0, 0.7 Hz, 1H), 8.16 (d, J=5.9 Hz, 1H), 8.03 (s, 1H), 7.69 (s, 1H), 7.60 (d, J=5.9 Hz, 1H), 7.48 (dd, J=5.0, 1.2 Hz, 1H), 4.40-4.29 (m, 1H), 3.93-3.86 (m, 1H), 3.74-3.60 (m, 3H), 2.94 (d, J=12.0 Hz, 1H), 1.22 (d, J=6.8 Hz, 3H), 0.87 (d, J=6.6 Hz, 3H).

### Example 191. Synthesis of tert-butyl (2R,5S)-4-(1-(4-cyanopyridin-2-yl)-3-(difluoromethyl)-1H-pyrrolo[3,2-c]pyridin-4-yl)-2,5-dimethylpiperazine-1-carboxylate (Compound 624)

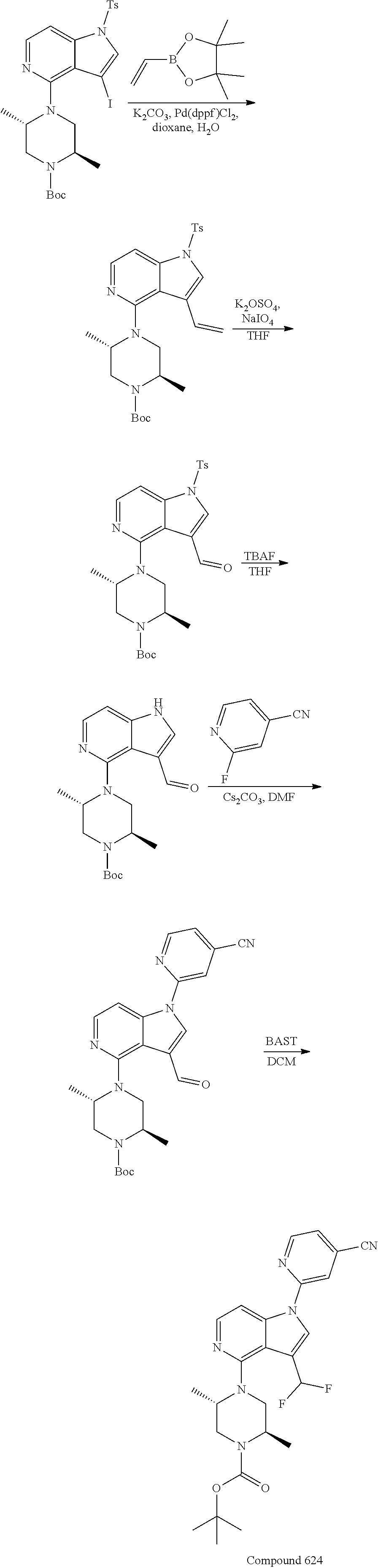

### Step 1. tert-Butyl (2R,5S)-2,5-dimethyl-4-(I-tosyl-3-vinyl-1H-pyrrolo[3,2-c]pyridin-4-yl)piperazine-1-carboxylate

To a solution of tert-butyl (2R,5S)-4-(3-iodo-1-tosyl-1H-pyrrolo[3,2-c]pyridin-4-yl)-2,5-dimethylpiperazine-1-carboxylate (330 mg, 0.54 mmol, prepared following the procedure outlined for compound 620) in dioxane (10 mL) and H₂O (2 mL) were added 4,4,5,5-tetramethyl-2-vinyl-1,3,2-dioxaborolane (0.18 mL, 1.1 mmol), K₂CO₃ (220 mg, 1.6 mmol) and Pd(dppf)Cl₂ (40 mg, 0.054 mmol), the resulting mixture was heated to 90° C. overnight. After cooling to room temperature, the reaction was concentrated, the residue was purified by flash column chromatography (silica gel, 0-20%, ethyl acetate in petroleum ether) to afford tert-butyl (2R,5S)-2,5-dimethyl-4-(1-tosyl-3-vinyl-1H-pyrrolo[3,2-c]pyridin-4-yl)piperazine-1-carboxylate (260 mg, 94%) as a white solid. LC/MS ESI (m/z): 511 (M+H)⁺.

### Step 2. tert-Butyl (2R,5S)-4-(3-formyl-1-tosyl-1H-pyrrolo[3,2-c]pyridin-4-yl)-2,5-dimethylpiperazine-1-carboxylate

To a solution of tert-butyl (2R,5S)-2,5-dimethyl-4-(1-tosyl-3-vinyl-1H-pyrrolo[3,2-c]pyridin-4-yl)piperazine-1-carboxylate (260 mg, 0.51 mmol) in THE (5 mL) were added K₂OsO₄ (19 mg, 0.051 mmol) and NaIO₄ (540 mg, 2.5 mmol). After stirring at room temperature overnight, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜30%, ethyl acetate in petroleum ether) to afford tert-butyl (2R,5S)-4-(3-formyl-1-tosyl-1H-pyrrolo[3,2-c]pyridin-4-yl)-2,5-dimethylpiperazine-1-carboxylate (100 mg, 38%) as a brown oil. LC/MS ESI (m/z): 513 (M+H)⁺.

### Step 3. tert-Butyl (2R,5S)-4-(3-formyl-1H-pyrrolo[3,2-c]pyridin-4-yl)-2,5-dimethylpiperazine-1-carboxylate

To a solution of tert-butyl (2R,5S)-4-(3-formyl-1-tosyl-1H-pyrrolo[3,2-c]pyridin-4-yl)-2,5-dimethylpiperazine-1-carboxylate (100 mg, 0.19 mmol) in THE (2 mL) was added TBAF (1.2 mL, 1.0 M in THF). After 1 h, the reaction was partitioned between EtOAc and water. The aqueous layer was extracted twice with EtOAc. The combined organic layers were washed with brine, dried over Na₂SO₄, filtered, and concentrated to afford tert-butyl (2R,5S)-4-(3-formyl-1H-pyrrolo[3,2-c]pyridin-4-yl)-2,5-dimethylpiperazine-1-carboxylate (65 mg, 93%) as a yellow oil. LC/MS ESI (m/z): 359 (M+H)⁺.

### Step 4. tert-Butyl (2R,5S)-4-(1-(4-cyanopyridin-2-yl)-3-formyl-1H-pyrrolo[3,2-c]pyridin-4-yl)-2,5-dimethylpiperazine-1-carboxylate

To a solution of tert-butyl (2R,5S)-4-(3-formyl-1H-pyrrolo[3,2-c]pyridin-4-yl)-2,5-dimethylpiperazine-1-carboxylate (65 mg, 0.18 mmol) in DMF (3 mL) were added 2-fluoroisonicotinonitrile (44 mg, 0.36 mmol) and Cs₂CO₃ (180 mg, 0.54 mmol). The resulting mixture was stirred at 50° C. for 1 h. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0-50%, ethyl acetate in petroleum ether) to afford tert-butyl (2R,5S)-4-(1-(4-cyanopyridin-2-yl)-3-formyl-1H-pyrrolo[3,2-c]pyridin-4-yl)-2,5-dimethylpiperazine-1-carboxylate (50 mg, 60%) as a brown oil. LC/MS ESI (m/z): 461 (M+H)⁺.

### Step 5. tert-Butyl (2R,5S)-4-(1-(4-cyanopyridin-2-yl)-3-(difluoromethyl)-1H-pyrrolo[3,2-c]pyridin-4-yl)-2,5-dimethylpiperazine-1-carboxylate

To a solution of tert-butyl (2R,5S)-4-(1-(4-cyanopyridin-2-yl)-3-formyl-1H-pyrrolo[3,2-c]pyridin-4-yl)-2,5-dimethylpiperazine-1-carboxylate (50 mg, 0.11 mmol) in DCM (2 mL) at 0° C. was added BAST (0.10 mL, 0.54 mmol). After stirring at room temperature over 3 days, the reaction was quenched with NaHCO₃ (aq.). The layers were separated, and the aqueous layer was extracted twice with DCM. The combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford tert-butyl (2R,5S)-4-(1-(4-cyanopyridin-2-yl)-3-(difluoromethyl)-1H-pyrrolo[3,2-c]pyridin-4-yl)-2,5-dimethylpiperazine-1-carboxylate (21 mg, 40%) as a white solid. LC/MS ESI (m/z): 483 (M+H)⁺. ¹H NMR (400 MHz, CDCl₃) δ 8.73 (d, J=5.0 Hz, 1H), 8.14 (d, J=5.9 Hz, 1H), 7.99-7.92 (m, 1H), 7.72 (d, J=5.7 Hz, 1H), 7.68 (s, 1H), 7.46 (d, J=5.1 Hz, 1H), 7.21 (t, J=55.4 Hz, 1H), 4.49-4.29 (m, 1H), 3.92-3.80 (m, 1H), 3.75 (d, J=13.0 Hz, 2H), 3.56 (dd, J=13.6, 2.6 Hz, 1H), 2.96 (d, J=12.1 Hz, 1H), 1.43 (s, 9H), 1.23 (d, J=6.8 Hz, 3H), 0.92 (d, J=6.1 Hz, 3H).

### Example 192. Synthesis of tert-butyl (2R,5S)-4-(7-cyclohexyl-5-(trifluoromethyl)-7H-pyrrolo[2,3-d]pyrimidin-4-yl)-2,5-dimethylpiperazine-1-carboxylate (Compound 625)

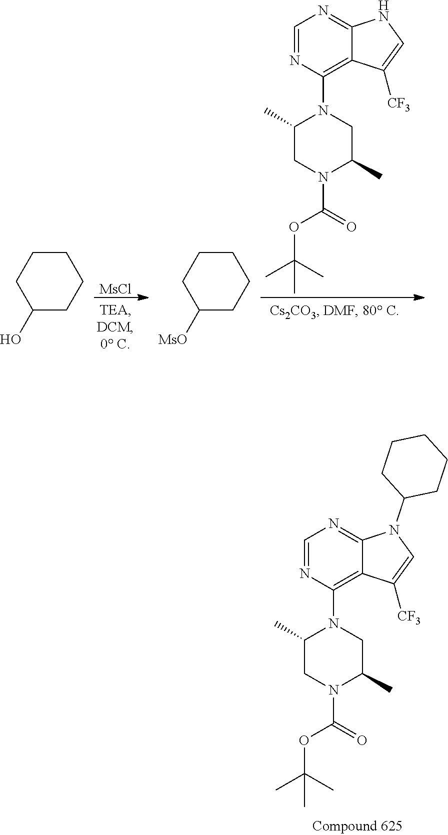

### Step 1. Cyclohexyl methanesulfonate

To a solution of cyclohexanol (300 mg, 3.0 mmol) and TEA (1.2 mL, 9.0 mmol) in DCM (5 mL) at 0° C. was added MsCl (0.46 mL, 6.0 mmol) dropwise. After 1 h, the reaction was partitioned between DCM and water. The aqueous layer was extracted twice with DCM. The combined organic layers were washed with brine, dried over Na₂SO₄, filtered, and concentrated to afford cyclohexyl methanesulfonate (500 mg, 93%) as a yellow oil.

### Step 2. tert-Butyl (2R,5S)-4-(7-cyclohexyl-5-(trifluoromethyl)-7H-pyrrolo[2,3-d]pyrimidin-4-yl)-2,5-dimethylpiperazine-1-carboxylate

To a solution of tert-butyl (2R,5S)-2,5-dimethyl-4-(5-(trifluoromethyl)-7H-pyrrolo[2,3-d]pyrimidin-4-yl)piperazine-1-carboxylate (50 mg, 0.12 mmol, prepared following the procedure outlined for compound 659) in DMF (3 mL) were added cyclohexyl methanesulfonate (45 mg, 0.25 mmol) and Cs₂CO₃ (120 mg, 0.37 mmol), the resulting mixture was stirred at 80° C. overnight. After cooling to room temperature, the reaction was partitioned between EtOAc and water. The aqueous layer was extracted twice with EtOAc. The combined organic layers were washed with brine, dried over Na₂SO₄, filtered and concentrated, the residue was purified by flash chromatography (silica gel, 0˜50%, ethyl acetate in petroleum ether) to give crude product which was further purified by prep-HPLC to afford tert-butyl (2R,5S)-4-(7-cyclohexyl-5-(trifluoromethyl)-7H-pyrrolo[2,3-d]pyrimidin-4-yl)-2,5-dimethylpiperazine-1-carboxylate (9.2 mg, 15%) as a white solid. LC/MS ESI (m/z): 482 (M+H)⁺. ¹H NMR (400 MHz, CDCl₃) δ 8.40 (s, 1H), 7.47 (s, 1H), 4.71-4.62 (m, 1H), 4.46-4.39 (m, 1H), 4.37-4.13 (m, 1H), 3.69-3.61 (m, 2H), 3.55 (dd, J=13.3, 3.1 Hz, 1H), 3.38 (d, J=13.3 Hz, 1H), 2.05 (m, 2H), 1.91-1.82 (m, 2H), 1.73 (d, J=13.2 Hz, 1H), 1.66-1.55 (m, 2H), 1.48-1.44 (m, 1H), 1.42 (s, 9H), 1.28-1.17 (m, 2H), 1.04 (m, 6H).

The following compound was prepared by the procedure analogous to the synthesis of compound 625 from the corresponding alcohol.

| Cmpd | | |
|---|---|---|
| No. | Chemical Name | LCMS and ¹H NMR |
| 626 | tert-butyl (2R,5S)-4-(7- | LC/MS ESI (m/z): 468 (M + H)⁺. ¹H NMR(400 MHz, |
| | cyclopentyl-5-(trifluoromethyl)- | CDCl₃) δ 8.40 (s, 1H), 7.45 (s, 1H), 5.14 (t, J = 7.2 Hz, 1H), |
| | 7H-pyrrolo[2,3-d]pyrimidin-4- | 4.43 (s, 1H), 4.30 (s, 1H), 3.64 (dd, J = 11.4, 7.3 Hz, 2H), |
| | yl)-2,5-dimethylpiperazine-1- | 3.55 (dd, J = 13.3, 3.4 Hz, 1H), 3.38 (d, J = 13.2 Hz, 1H), |
| | carboxylate | 2.25-2.15 (m, 2H), 1.86-1.72 (m, 6H), 1.42 (s, 9H), 1.04 |
| | | (m, 6H). |

### Example 193. Synthesis of tert-butyl (2R,5S)-4-(7-cyclohexyl-5-(difluoromethyl)-7H-pyrrolo[2,3-d]pyrimidin-4-yl)-2,5-dimethylpiperazine-1-carboxylate (Compound 627)

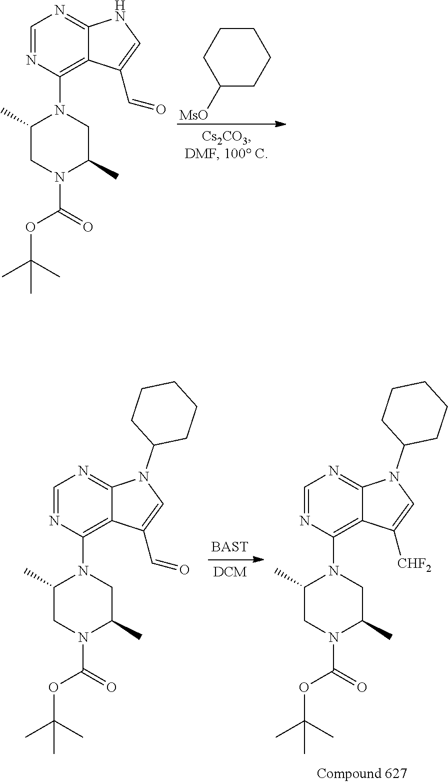

### Step 1. tert-Butyl (2R,5S)-4-(7-cyclohexyl-5-formyl-7H-pyrrolo[2,3-d]pyrimidin-4-yl)-2,5-dimethylpiperazine-1-carboxylate

To a solution of tert-butyl (2R,5S)-4-(5-formyl-7H-pyrrolo[2,3-d]pyrimidin-4-yl)-2,5-dimethylpiperazine-1-carboxylate (50 mg, 0.14 mmol, prepared following the procedure outlined for compound 630) in DMF (3 mL) were added cyclohexyl methanesulfonate (50 mg, 0.28 mmol, prepared following the procedure outlined for compound 625) and Cs₂CO₃ (140 mg, 0.41 mmol). The resulting mixture was stirred at 100° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford tert-butyl (2R,5S)-4-(7-cyclohexyl-5-formyl-7H-pyrrolo[2,3-d]pyrimidin-4-yl)-2,5-dimethylpiperazine-1-carboxylate (20 mg, 32%) as a yellow oil. LC/MS ESI (m/z): 442 (M+H)⁺.

### Step 2. tert-Butyl (2R,5S)-4-(7-cyclohexyl-5-(difluoromethyl)-7H-pyrrolo[2,3-d]pyrimidin-4-yl)-2,5-dimethylpiperazine-1-carboxylate

To a solution of tert-butyl (2R,5S)-4-(7-cyclohexyl-5-formyl-7H-pyrrolo[2,3-d]pyrimidin-4-yl)-2,5-dimethylpiperazine-1-carboxylate (20 mg, 0.045 mmol) in DCM (2 mL) at 0° C. was added BAST (0.08 mL, 0.45 mmol). After stirring at 30° C. overnight, the reaction was quenched with NaHCO₃ (aq.). The layers were separated, and the aqueous layer was extracted twice with DCM. The combined organic layers were washed with brine, dried over Na₂SO₄, concentrated, and purified by prep-HPLC to afford tert-butyl (2R,5S)-4-(7-cyclohexyl-5-(difluoromethyl)-7H-pyrrolo[2,3-d]pyrimidin-4-yl)-2,5-dimethylpiperazine-1-carboxylate (5.7 mg, 27%) as a white solid. LC/MS ESI (m/z): 464 (M+H)⁺. ¹H NMR (400 MHz, CDCl₃) δ 8.40 (s, 1H), 7.40-7.33 (m, 1H), 6.86 (t, J=56.2 Hz, 1H), 4.69-4.59 (m, 1H), 4.45-4.39 (m, 1H), 4.37-4.16 (m, 1H), 3.72-3.61 (m, 2H), 3.52 (dd, J=13.3, 3.4 Hz, 1H), 3.41 (d, J=13.0 Hz, 1H), 2.07 (d, J=12.2 Hz, 1H), 2.00 (d, J=12.1 Hz, 1H), 1.91-1.80 (m, 2H), 1.73 (d, J=13.1 Hz, 1H), 1.66-1.59 (m, 1H), 1.50-1.44 (m, 2H), 1.42 (s, 9H), 1.27-1.14 (m, 2H), 1.06 (m, 6H).

The following compound was prepared by the procedure analogous to the synthesis of compound 625 from the corresponding alcohol.

| Cmpd | | |
|---|---|---|
| No. | Chemical Name | LCMS and ¹H NMR |
| 628 | tert-butyl (2R,5S)-4-(7- | LC/MS ESI (m/z): 450 (M + H)⁺. ¹H NMR(400 MHz, |
| | cyclopentyl-5-(difluoromethyl)- | CDCl3) δ 8.39 (s, 1H), 7.36 (s, 1H), 6.86 (t, J = 56.2 Hz, |
| | 7H-pyrrolo[2,3-d]pyrimidin-4- | 1H), 5.18-5.10 (m, 1H), 4.47-4.39 (m, 1H), 4.31 (s, 1H), |
| | yl)-2,5-dimethylpiperazine-1- | 3.72-3.63 (m, 2H), 3.52 (dd, J = 13.3, 3.5 Hz, 1H), 3.42 |
| | carboxylate | (d, J = 12.9 Hz, 1H), 2.26-2.12 (m, 2H), 1.90-1.69 (m, |
| | | 6H), 1.42 (s, 9H), 1.09-1.04 (m, 6H). |

### Example 194. Synthesis of tert-butyl (2R,5S)-4-(7-(3-cyanocyclohexyl)-5-(trifluoromethyl)-7H-pyrrolo[2,3-d]pyrimidin-4-yl)-2,5-dimethylpiperazine-1-carboxylate (Compound 629)

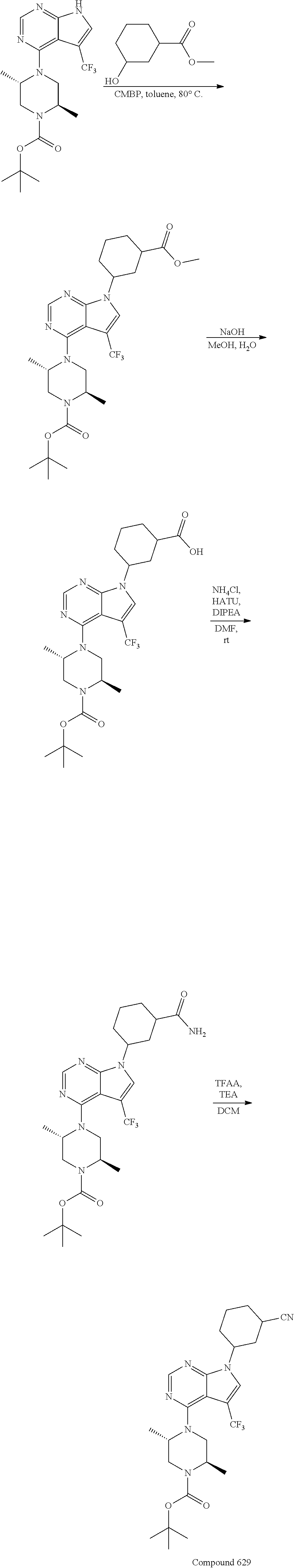

### Step 1. tert-Butyl (2R,5S)-4-(7-(3-(methoxycarbonyl)cyclohexyl)-5-(trifluoromethyl)-7H-pyrrolo[2,3-d]pyrimidin-4-yl)-2,5-dimethylpiperazine-1-carboxylate

To a solution of tert-butyl (2R,5S)-2,5-dimethyl-4-(5-(trifluoromethyl)-7H-pyrrolo[2,3-d]pyrimidin-4-yl)piperazine-1-carboxylate (100 mg, 0.25 mmol, prepared following the procedure outlined for compound 659) in toluene (3 mL) were added methyl 3-hydroxycyclohexane-1-carboxylate (120 mg, 0.75 mmol) and (cyanomethylene)tributylphosphorane (CMBP. 0.14 mL, 0.50 mmol). The resulting mixture was stirred at 80° C. overnight. After cooling to room temperature, the reaction was concentrated and the residue was purified by flash column chromatography (silica gel, 0˜30%, ethyl acetate in petroleum ether) to afford tert-butyl (2R,5S)-4-(7-(3-(methoxycarbonyl)cyclohexyl)-5-(trifluoromethyl)-7H-pyrrolo[2,3-d]pyrimidin-4-yl)-2,5-dimethylpiperazine-1-carboxylate (120 mg, 88%) as a yellow oil. LC/MS ESI (m/z): 540 (M+H)⁺.

### Step 2. 3-(4-((2S,5R)-4-(tert-Butoxycarbonyl)-2,5-dimethylpiperazin-1-yl)-5-(trifluoromethyl)-7H-pyrrolo[2,3-d]pyrimidin-7-yl)cyclohexane-1-carboxylic acid

To a solution of tert-butyl (2R,5S)-4-(7-(3-(methoxycarbonyl)cyclohexyl)-5-(trifluoromethyl)-7H-pyrrolo[2,3-d]pyrimidin-4-yl)-2,5-dimethylpiperazine-1-carboxylate (120 mg, 0.22 mmol) in MeOH (5 mL) and H₂O (1 mL) was added NaOH (18 mg, 0.44 mmol). The resulting mixture was stirred at 40° C. overnight. After cooling to room temperature, MeOH was removed. The residue was acidified with 1 M HCl to pH 6 and extracted twice with DCM. The combined organic layers were washed with brine, dried over Na₂SO₄, filtered and concentrated. The residue was purified by flash column chromatography (silica gel, 0˜50%, ethyl acetate in petroleum ether) to afford 3-(4-((2S,5R)-4-(tert-butoxycarbonyl)-2,5-dimethylpiperazin-1-yl)-5-(trifluoromethyl)-7H-pyrrolo[2,3-d]pyrimidin-7-yl)cyclohexane-1-carboxylic acid (40 mg, 34%) as a yellow oil. LC/MS ESI (m/z): 526 (M+H)⁺.

### Step 3. tert-Butyl (2R,5S)-4-(7-(3-carbamoylcyclohexyl)-5-(trifluoromethyl)-7H-pyrrolo[2,3-d]pyrimidin-4-yl)-2,5-dimethylpiperazine-1-carboxylate

To a solution of 3-(4-((2S,5R)-4-(tert-butoxycarbonyl)-2,5-dimethylpiperazin-1-yl)-5-(trifluoromethyl)-7H-pyrrolo[2,3-d]pyrimidin-7-yl)cyclohexane-1-carboxylic acid (40 mg, 0.076 mmol) in DMF (2 mL) were added NH₄Cl (8.1 mg, 0.15 mmol), HATU (29 mg, 0.076 mmol) and DIPEA (0.04 mL, 0.22 mmol). After stirring at room temperature overnight, the reaction was partitioned between EtOAc and water. The aqueous layer was extracted twice with EtOAc. The combined organic layers were washed with brine, dried over Na₂SO₄, filtered, and concentrated to afford tert-butyl (2R,5S)-4-(7-(3-carbamoylcyclohexyl)-5-(trifluoromethyl)-7H-pyrrolo[2,3-d]pyrimidin-4-yl)-2,5-dimethylpiperazine-1-carboxylate (35 mg, 87%) as a yellow oil. LC/MS ESI (m/z): 525 (M+H)⁺.

### Step 4. tert-Butyl (2R,5S)-4-(7-(3-cyanocyclohexyl)-5-(trifluoromethyl)-7H-pyrrolo[2,3-d]pyrimidin-4-yl)-2,5-dimethylpiperazine-1-carboxylate

To a solution of tert-butyl (2R,5S)-4-(7-(3-carbamoylcyclohexyl)-5-(trifluoromethyl)-7H-pyrrolo[2,3-d]pyrimidin-4-yl)-2,5-dimethylpiperazine-1-carboxylate (35 mg, 0.067 mmol) and TEA (0.08 mL, 0.60 mmol) in DCM (2 mL) at 0° C. was added TFAA (35 mg, 0.16 mmol). After stirring at 0° C. for 20 min, the reaction was quenched with water. The layers were separated, and the aqueous layer was extracted twice with DCM. The combined organic layers were washed with brine, dried over Na₂SO₄, concentrated, and purified by prep-HPLC to afford tert-butyl (2R,5S)-4-(7-(3-cyanocyclohexyl)-5-(trifluoromethyl)-7H-pyrrolo[2,3-d]pyrimidin-4-yl)-2,5-dimethylpiperazine-1-carboxylate (21 mg, 600%) as a white solid. LC/MS ESI (m/z): 507 (M+H)⁺. ¹H NMR (400 MHz, CDCl₃) δ 8.43-8.33 (m, 1H), 7.41 (d, J=8.9 Hz, 111), 4.97-4.62 (m, 1H), 4.51-4.21 (m, 2H), 3.72-3.59 (m, 21H), 3.54 (dd, J=13.3, 3.1 Hz, 11H), 3.46-3.30 (m, 1H), 3.20-2.62 (m, 1H), 2.53-2.27 (m, 1H), 2.18-1.99 (m, 3H), 1.94-1.69 (m, 2H), 1.65-1.53 (m, 2H), 1.42 (s, 9H), 1.10-0.96 (m, 6.).

The following compounds were prepared by procedures analogous to the synthesis of compound 629 from the corresponding alcohols (compound 631 and compound 632 were made from tetrahydro-2H-pyran-3-ol and separated via SFC chiral separation at the last step).

| Cmpd | | |
|---|---|---|
| No. | Chemical Name | LCMS and ¹H NMR |
| 630 | tert-butyl (2R,5S)-2,5- | LC/MS ESI (m/z): 484 (M + H)⁺. ¹H NMR (400 MHz, |
| | dimethyl-4-(7-(tetrahydro-2H- | CDCl₃) δ 8.39 (s, 1H), 7.47 (s, 1H), 4.98-4.87 (m, 1H), |
| | pyran-4-yl)-5- | 4.45 (s, 1H), 4.40-4.18 (m, 1H), 4.11-4.04 (m, 2H), 3.69- |
| | (trifluoromethyl)-7H- | 3.52 (m, 5H), 3.39 (d, J = 13.3 Hz, 1H), 2.06-1.90 (m, |
| | pyrrolo[2,3-d]pyrimidin-4- | 4H), 1.42 (s, 9H), 1.04 (m, 6H). |
| | yl)piperazine-1-carboxylate | |
| 631 | tert-butyl (2R,5S)-2,5- | LC/MS ESI (m/z): 484 (M + H)⁺. ¹H NMR (400 MHz, |
| | dimethyl-4-(7-((S)-tetrahydro- | CDCl₃) δ 8.39 (s, 1H), 7.73 (s, 1H), 4.89-4.77 (m, 1H), |
| | 2H-pyran-3-yl)-5- | 4.45 (s, 1H), 4.38-4.18 (m, 1H), 3.99 (dd, J = 11.5, 3.5 |
| | (trifluoromethyl)-7H- | Hz, 1H), 3.82-3.62 (m, 5H), 3.55 (dd, J = 13.3, 3.4 Hz, |
| | pyrrolo[2,3-d]pyrimidin-4- | 1H), 3.39 (d, J = 13.3 Hz, 1H), 2.16-1.98 (m, 2H), 1.82- |
| | yl)piperazine-1-carboxylate | 1.66 (m, 2H), 1.42 (s, 9H), 1.04 (m, 6H). |
| 632 | tert-butyl (2R,5S)-2,5- | LC/MS ESI (m/z): 484 (M + H)⁺. ¹H NMR (400 MHz, |
| | dimethyl-4-(7-((R)-tetrahydro- | CDCl₃) δ 8.39 (s, 1H), 7.74 (s, 1H), 4.88-4.81 (m, 1H), |
| | 2H-pyran-3-yl)-5- | 4.44 (s, 1H), 4.39-4.18 (m, 1H), 4.04 (dd, J = 11.5, 3.4 |
| | (trifluoromethyl)-7H- | Hz, 1H), 3.83-3.76 (m, 2H), 3.71-3.62 (m, 3H), 3.55 |
| | pyrrolo[2,3-d]pyrimidin-4- | (dd, J = 13.3, 3.4 Hz, 1H), 3.38 (d, J = 13.2 Hz, 1H), 2.13- |
| | yl)piperazine-1-carboxylate | 1.98 (m, 2H), 1.77-1.64 (m, 2H), 1.42 (s, 9H), 1.04 (m, |
| | | 6H). |

### Example 195. Synthesis of tert-butyl (2R,5S)-4-(7-(3-cyanocyclohexyl)-5-(difluoromethyl)-7H-pyrrolo[2,3-d]pyrimidin-4-yl)-2,5-dimethylpiperazine-1-carboxylate (Compound 633)

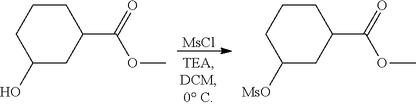

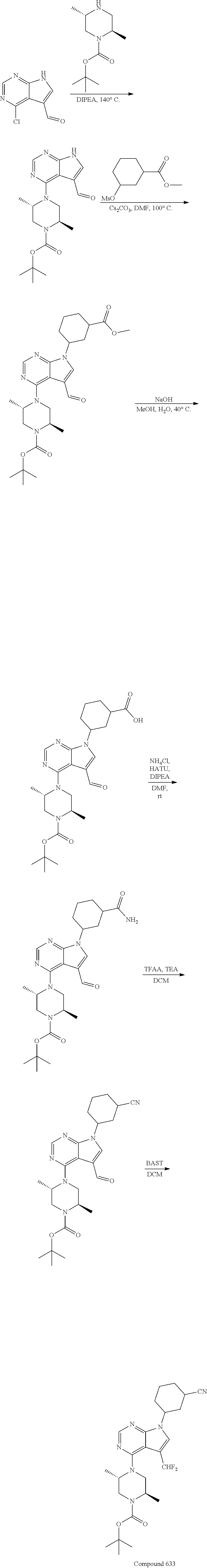

### Step 1. Methyl 3-((methylsulfonyl)oxy)cyclohexane-1-carboxylate

To a solution of methyl 3-hydroxycyclohexane-1-carboxylate (200 mg, 1.3 mmol) and TEA (0.53 mL, 3.8 mmol) in DCM (3 mL) at 0° C. was added MsCl (0.20 mL, 2.5 mmol). After stirring at room temperature for 2 h, the reaction was quenched with ice water. The aqueous layer was extracted twice with DCM. The combined organic layers were washed with brine, dried over Na₂SO₄, and concentrated to afford methyl 3-((methylsulfonyl)oxy)cyclohexane-1-carboxylate (290 mg, 97%) as a yellow oil.

### Step 2. tert-Butyl (2R,5S)-4-(5-formyl-7H-pyrrolo[2,3-d]pyrimidin-4-yl)-2,5-dimethylpiperazine-1-carboxylate

To a solution of 4-chloro-7H-pyrrolo[2,3-d]pyrimidine-5-carbaldehyde (1.0 g, 5.5 mmol) in DIPEA (5 mL) was added tert-butyl (2R,5S)-2,5-dimethylpiperazine-1-carboxylate (2.4 g, 11 mmol). The resulting mixture was stirred at 140° C. for 1 h. After cooling to room temperature, the reaction was concentrated and the residue was purified by flash column chromatography (silica gel, 0˜90%, ethyl acetate in petroleum ether) to afford tert-butyl (2R,5S)-4-(5-formyl-7H-pyrrolo[2,3-d]pyrimidin-4-yl)-2,5-dimethylpiperazine-1-carboxylate (1.3 g, 67%) as a yellow solid. LC/MS ESI (m/z): 360 (M+H)⁺.

### Step 3. tert-Butyl (2R,5S)-4-(5-formyl-7-(3-(methoxycarbonyl)cyclohexyl)-7H-pyrrolo[2,3-d]pyrimidin-4-yl)-2,5-dimethylpiperazine-1-carboxylate

To a solution of tert-butyl (2R,5S)-4-(5-formyl-7H-pyrrolo[2,3-d]pyrimidin-4-yl)-2,5-dimethylpiperazine-1-carboxylate (200 mg, 0.55 mmol) in DMF (5 mL) were added methyl 3-((methylsulfonyl)oxy)cyclohexane-1-carboxylate (260 mg, 1.1 mmol) and Cs₂CO₃ (540 mg, 1.7 mmol). The resulting mixture was stirred at 100° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford crude tert-butyl (2R,5S)-4-(5-formyl-7-(3-(methoxycarbonyl)cyclohexyl)-7H-pyrrolo[2,3-d]pyrimidin-4-yl)-2,5-dimethylpiperazine-1-carboxylate (130 mg, 46%) as a yellow oil. LC/MS ESI (m/z): 500 (M+H)⁺.

### Step 4. 3-(4-((2S,5R)-4-(tert-Butoxycarbonyl)-2,5-dimethylpiperazin-1-yl)-5-formyl-7H-pyrrolo[2,3-d]pyrimidin-7-yl)cyclohexane-1-carboxylic acid

To a solution of tert-butyl (2R,5S)-4-(5-formyl-7-(3-(methoxycarbonyl)cyclohexyl)-7H-pyrrolo[2,3-d]pyrimidin-4-yl)-2,5-dimethylpiperazine-1-carboxylate (130 mg, 0.26 mmol) in MeOH (5 mL) and H₂O (1 mL) was added NaOH (21 mg, 0.52 mmol). The resulting mixture was stirred at 40° C. overnight. After cooling to room temperature, methanol was removed. The residue was acidified by 1 N HCl to pH 6 and the mixture was extracted twice with DCM. The combined organic layers were washed with brine, dried over Na₂SO₄, filtered, and concentrated to afford crude 3-(4-((2S,5R)-4-(tert-butoxycarbonyl)-2,5-dimethylpiperazin-1-yl)-5-formyl-7H-pyrrolo[2,3-d]pyrimidin-7-yl)cyclohexane-1-carboxylic acid (60 mg, 47%) as a yellow oil. LC/MS ESI (m/z): 486 (M+H)⁺.

### Step 5. tert-Butyl (2R,5S)-4-(7-(3-carbamoylcyclohexyl)-5-formyl-7H-pyrrolo[2,3-d]pyrimidin-4-yl)-2,5-dimethylpiperazine-1-carboxylate

To a solution of 3-(4-((2S,5R)-4-(tert-butoxycarbonyl)-2,5-dimethylpiperazin-1-yl)-5-formyl-7H-pyrrolo[2,3-d]pyrimidin-7-yl)cyclohexane-1-carboxylic acid (60 mg, 0.12 mmol) in DMF (3 mL) were added NH₄Cl (13 mg, 0.24 mmol), HATU (47 mg, 0.12 mmol) and DIPEA (0.07 mL, 0.37 mmol). After stirring at room temperature overnight, the reaction was partitioned between EtOAc and water. The aqueous layer was extracted twice with EtOAc twice and the combined organic layers were washed with brine, dried over Na₂SO₄, filtered, and concentrated to afford tert-butyl (2R,5S)-4-(7-(3-carbamoylcyclohexyl)-5-formyl-7H-pyrrolo[2,3-d]pyrimidin-4-yl)-2,5-dimethylpiperazine-1-carboxylate (55 mg, 91%) as a yellow oil. LC/MS ESI (m/z): 485 (M+H)⁺.

### Step 6. tert-Butyl (2R,5S)-4-(7-(3-cyanocyclohexyl)-5-formyl-7H-pyrrolo[2,3-d]pyrimidin-4-yl)-2,5-dimethylpiperazine-1-carboxylate

To a solution of tert-butyl (2R,5S)-4-(7-(3-carbamoylcyclohexyl)-5-formyl-7H-pyrrolo[2,3-d]pyrimidin-4-yl)-2,5-dimethylpiperazine-1-carboxylate (55 mg, 0.11 mmol) and TEA (0.14 mL, 1.0 mmol) in DCM (3 mL) at 0° C. was added TFAA (60 mg, 0.28 mmol). After stirring at 0° C. for 30 min, the reaction was quenched with water. The layers were separated, and the aqueous layer was extracted twice with DCM. The combined organic layers were washed with brine, dried over Na₂SO₄, concentrated, and purified by flash column chromatography (silica gel, ethyl acetate in petroleum ether) to afford tert-butyl (2R,5S)-4-(7-(3-cyanocyclohexyl)-5-formyl-7H-pyrrolo[2,3-d]pyrimidin-4-yl)-2,5-dimethylpiperazine-1-carboxylate (50 mg, 94%) as a yellow oil. LC/MS ESI (m/z): 467 (M+H)⁺.

### Step 7. tert-Butyl (2R,5S)-4-(7-(3-cyanocyclohexyl)-5-(difluoromethyl)-7H-pyrrolo[2,3-d]pyrimidin-4-yl)-2,5-dimethylpiperazine-1-carboxylate

To a solution of tert-butyl (2R,5S)-4-(7-(3-cyanocyclohexyl)-5-formyl-7H-pyrrolo[2,3-d]pyrimidin-4-yl)-2,5-dimethylpiperazine-1-carboxylate (50 mg, 0.11 mmol) in DCM (3 mL) at 0° C. was added BAST (0.10 mL, 0.53 mmol). After stirring at 30° C. for three days, the reaction was partitioned between DCM and NaHCO₃ (aq.). The aqueous layer was extracted twice with DCM. The combined organic layers were washed with brine, dried over Na₂SO₄, filtered and concentrated. The residue was purified by prep-HPLC to afford tert-butyl (2R,5S)-4-(7-(3-cyanocyclohexyl)-5-(difluoromethyl)-7H-pyrrolo[2,3-d]pyrimidin-4-yl)-2,5-dimethylpiperazine-1-carboxylate (5.5 mg, 10%) as a yellow solid. LC/MS ESI (m/z): 489 (M+H)⁺. ¹H NMR (400 MHz, CDCl₃) δ 8.40-8.34 (m, 1H), 7.31 (d, J=7.9 Hz, 1H), 6.85 (t, J=56.1 Hz, 1H), 4.92-4.59 (m, 1H), 4.51-4.24 (m, 2H), 3.74-3.62 (m, 2H), 3.58-3.13 (m, 3H), 2.76-2.19 (m, 2H), 2.16-2.00 (m, 2H), 1.96-1.77 (m, 2H), 1.74-1.54 (m, 2H), 1.42 (s, 9H), 1.10-1.01 (m, 6H).

The following compound was prepared by a procedure analogous to the synthesis of compound 633 from 3-hydroxycyclopentane-1-carbonitrile.

| Cmpd | | |
|---|---|---|
| No. | Chemical Name | LCMS and ¹H NMR |
| 634 | tert-butyl (2R,5S)-4-(7-(3- | LC/MS ESI (m/z): 475 (M + H)⁺. ¹H NMR (400 MHz, |
| | cyanocyclopentyl)-5- | CDCl₃) δ 8.43 (s, 1H), 7.35-7.27 (m, 1H), 6.90 (t, J = 56.1 |
| | (difluoromethyl)-7H- | Hz, 1H), 5.31-5.19 (m, 1H), 4.58-4.30 (m, 2H), 3.78- |
| | pyrrolo[2,3-d]pyrimidin-4-yl)- | 3.70 (m, 2H), 3.62-3.54 (m, 1H), 3.49 (d, J = 13.0 Hz, |
| | 2,5-dimethylpiperazine-1- | 1H), 3.40-3.30 (m, 1H), 2.65-2.34 (m, 4H), 2.22-2.04 |
| | carboxylate | (m, 2H), 1.49 (s, 9H), 1.13 (m, 6H). |

### Example 196. Synthesis of tert-butyl (2R,5S)-4-(5-(azetidin-1-yl)-7-(3-fluorophenyl)-7H-pyrrolo[2,3-d]pyrimidin-4-yl)-2,5-dimethylpiperazine-1-carboxylate (Compound 635)

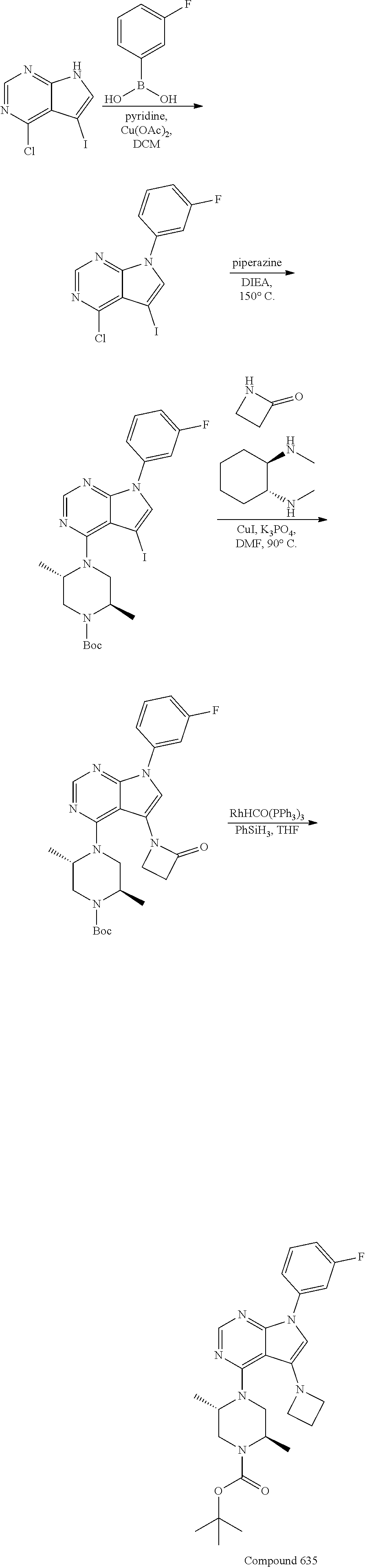

### Step 1. 4-Chloro-7-(3-fluorophenyl)-5-iodo-7H-pyrrolo[2,3-d]pyrimidine

To a solution of 4-chloro-5-iodo-7H-pyrrolo[2,3-d]pyrimidine (3.0 g, 11 mmol) in DCM (50 mL) were added (3-fluorophenyl)boronic acid (2.2 g, 16 mmol), Cu(OAc)₂ (5.3 g, 27 mmol) and pyridine (1.7 mL, 21 mmol). The resulting mixture was stirred at room temperature under O₂ atmosphere overnight. Aqueous ammonia (80 mL) was added, and the reaction was filtered. The filtrate was partitioned between DCM and water. The aqueous phase was extracted twice with DCM. The combined organic layers were washed with brine, dried over Na₂SO₄, filtered and concentrated. The residue was purified by flash column chromatography (silica gel, 0˜30% ethyl acetate in petroleum ether) to afford 4-chloro-7-(3-fluorophenyl)-5-iodo-7H-pyrrolo[2,3-d]pyrimidine (2.6 g, 66%) as a white solid. LC/MS ESI (m/z): 374 (M+H)⁺.

### Step 2. tert-Butyl (2R,5S)-4-(7-(3-fluorophenyl)-5-iodo-7H-pyrrolo[2,3-d]pyrimidin-4-yl)-2,5-dimethylpiperazine-1-carboxylate

A mixture of 4-chloro-7-(3-fluorophenyl)-5-iodo-7H-pyrrolo[2,3-d]pyrimidine (2.6 g, 6.9 mmol), tert-butyl (2R,5S)-2,5-dimethylpiperazine-1-carboxylate (2.2 g, 10 mmol) and DIPEA (15 mL) was stirred at 150° C. for 1 h. After cooling to room temperature, the solvent was removed and the residue was purified by flash column chromatography (silica gel, 0˜30% ethyl acetate in petroleum ether) to afford tert-butyl (2R,5S)-4-(7-(3-fluorophenyl)-5-iodo-7H-pyrrolo[2,3-d]pyrimidin-4-yl)-2,5-dimethylpiperazine-1-carboxylate (2.7 g, 72%) as a white solid. LC/MS ESI (m/z): 552 (M+H)⁺.

### Step 3. tert-Butyl (2R,5S)-4-(7-(3-fluorophenyl)-5-(2-oxoazetidin-1-yl)-7H-pyrrolo[2,3-d]pyrimidin-4-yl)-2,5-dimethylpiperazine-1-carboxylate

To a solution of tert-butyl (2R,5S)-4-(7-(3-fluorophenyl)-5-iodo-7H-pyrrolo[2,3-d]pyrimidin-4-yl)-2,5-dimethylpiperazine-1-carboxylate (800 mg, 1.4 mmol) in DMF (15 mL) were added azetidin-2-one (400 mg, 5.6 mmol), trans-N,N′-dimethylcyclohexane-1,2-diamine (200 mg, 1.4 mmol), CuI (130 mg, 0.70 mmol) and K₃PO₄ (590 mg, 2.8 mmol). The resulting mixture was heated to 90° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0˜100%, ethyl acetate in petroleum ether) to afford tert-butyl (2R,5S)-4-(7-(3-fluorophenyl)-5-(2-oxoazetidin-1-yl)-7H-pyrrolo[2,3-d]pyrimidin-4-yl)-2,5-dimethylpiperazine-1-carboxylate (500 mg, 72%) as a white solid. LC/MS ESI (m/z): 495 (M+H)⁺.

### Step 4. tert-Butyl (2R,5S)-4-(5-(azetidin-1-yl)-7-(3-fluorophenyl)-7H-pyrrolo[2,3-d]pyrimidin-4-yl)-2,5-dimethylpiperazine-1-carboxylate

To a solution of tert-butyl (2R,5S)-4-(7-(3-fluorophenyl)-5-(2-oxoazetidin-1-yl)-7H-pyrrolo[2,3-d]pyrimidin-4-yl)-2,5-dimethylpiperazine-1-carboxylate (500 mg, 1.0 mmol) in THE (8 mL) at 0° C. were added RhHCO(PPh₃)₃ (92 mg, 0.10 mmol) and PhSiH₃ (0.60 mL, 5.0 mmol). After stirring at room temperature for 1 h under N₂, the reaction was partitioned between EtOAc and water. The aqueous layer was extracted twice with EtOAc, and the combined organic layers were washed with brine, dried over Na₂SO₄, filtered and concentrated. The residue was purified by flash column chromatography (silica gel, 0˜35% ethyl acetate in petroleum ether) to give crude product which was further purified by prep-HPLC to afford tert-butyl (2R,5S)-4-(5-(azetidin-1-yl)-7-(3-fluorophenyl)-7H-pyrrolo[2,3-d]pyrimidin-4-yl)-2,5-dimethylpiperazine-1-carboxylate (470 mg, 98%) as a white solid. LC/MS ESI (m/z): 481 (M+H)⁺. ¹H NMR (400 MHz, CDCl₃) δ 8.36 (s, 1H), 7.57-7.53 (m, 2H), 7.46-7.41 (m, 1H), 7.02-6.96 (m, 1H), 6.61 (s, 1H), 5.12 (s, 1H), 4.42 (s, 1H), 4.00 (d, J=13.5 Hz, 1H), 3.83-3.57 (m, 7H), 2.32-2.26 (m, 2H), 1.49 (s, 9H), 1.14 (d, J=6.7 Hz, 3H), 1.03 (d, J=6.8 Hz, 3H).

The following compound was prepared by the procedure analogous to the synthesis of compound 635 from the corresponding chiral amine.

| Cmpd | | |
|---|---|---|
| No. | Chemical Name | LCMS and ¹H NMR |
| 636 | tert-butyl (S)-4-(5-(azetidin-1- | LC/MS ESI (m/z): 467 (M + H)⁺. ¹H NMR (400 MHz, |
| | yl)-7-(3-fluorophenyl)-7H- | CDCl₃) δ 8.31 (s, 1H), 7.50-7.43 (m, 2H), 7.41-7.33 (m, |
| | pyrrolo[2,3-d]pyrimidin-4-yl)-3- | 1H), 6.95-6.89 (m, 1H), 6.55 (s, 1H), 4.95-4.83 (m, 1H), |
| | methylpiperazine-1-carboxylate | 4.13-3.79 (m, 3H), 3.74-3.67 (m, 2H), 3.56 (q, J = 6.8 |
| | | Hz, 2H), 3.50-3.30 (m, 2H), 3.03-2.84 (m, 1H), 2.26- |
| | | 2.17 (m, 2H), 1.43 (s, 9H), 1.09 (d, J = 6.6 Hz, 3H). |

### Example 197. Synthesis of tert-butyl (S)-4-(3-(azetidin-1-yl)-1-(3-fluorophenyl)-1H-pyrrolo[3,2-c]pyridin-4-yl)-3-methylpiperazine-1-carboxylate (Compound 637)

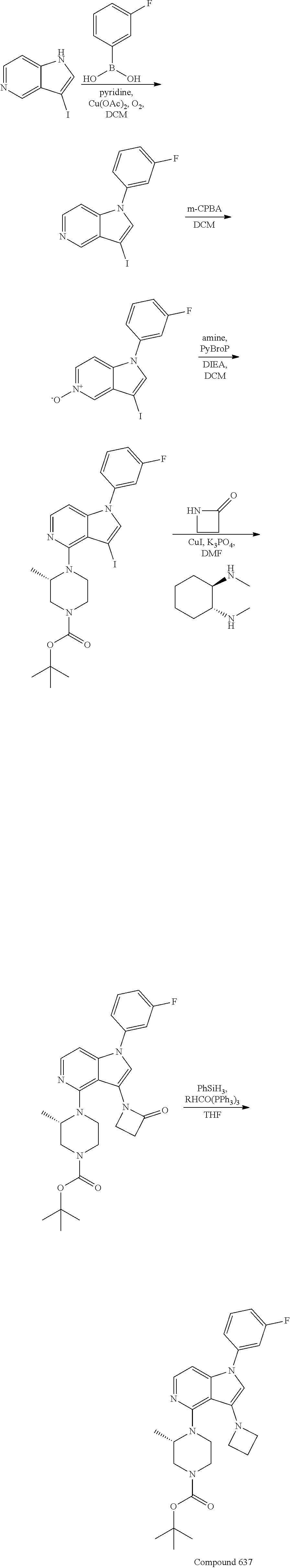

### Step 1. 1-(3-Fluorophenyl)-3-iodo-1H-pyrrolo[3,2-c]pyridine

A mixture of 3-iodo-1H-pyrrolo[3,2-c]pyridine (2.9 g, 12 mmol), (3-fluorophenyl)boronic acid (4.9 g, 35 mmol), Cu(OAc)₂ (5.8 g, 30 mmol) and pyridine (5.7 mL, 71 mmol) in DCM (50 mL) was stirred at room temperature for 48 h under oxygen atmosphere. The reaction mixture was treated with aqueous ammonia and was filtered. The filtrate was extracted twice with DCM. The combined organic layers were washed with brine, dried over Na₂SO₄, filtered and concentrated. The residue was purified by column chromatography on silica gel (0˜30% ethyl acetate in petroleum ether) to give 1-(3-fluorophenyl)-3-iodo-1H-pyrrolo[3,2-c]pyridine (1.5 g, 37%) as a solid. LC/MS ESI (m/z): 339 (M+H)⁺.

### Step 2. 1-(3-Fluorophenyl)-3-iodo-1H-pyrrolo[3,2-c]pyridine 5-oxide

To a solution of 1-(3-fluorophenyl)-3-iodo-1H-pyrrolo[3,2-c]pyridine (1.5 g, 4.4 mmol) in DCM (20 mL) at 0° C. was added 3-chloroperoxybenzoic acid (1.1 g, 6.6 mmol) in portions. The resulting mixture was stirred at room temperature overnight. The reaction was quenched with NaHCO₃ (aq.) and extracted twice with DCM. The combined organic layers were washed with brine, dried over Na₂SO₄, filtered and concentrated. The residue was purified by flash column chromatography (silica gel, 0-20% MeOH in DCM) to provide 1-(3-fluorophenyl)-3-iodo-1H-pyrrolo[3,2-c]pyridine 5-oxide (920 mg, 61%) as a light yellow solid. LC/MS ESI (m/z): 355 (M+H)⁺.

### Step 3. tert-Butyl (S)-4-(1-(3-fluorophenyl)-3-iodo-1H-pyrrolo[3,2-c]pyridin-4-yl)-3-methylpiperazine-1-carboxylate

To a solution of 1-(3-fluorophenyl)-3-iodo-1H-pyrrolo[3,2-c]pyridine 5-oxide (450 mg, 1.3 mmol) in DCM (10 mL) were added tert-butyl (S)-3-methylpiperazine-1-carboxylate (1.3 g, 6.4 mmol), DIPEA (0.90 mL, 5.1 mmol) and PyBroP (1.2 g, 2.5 mmol). After stirring at room temperature for 3 days, the reaction was partitioned between DCM and water. The aqueous layer was extracted twice with DCM. The combined organic layers were washed with brine, dried over Na₂SO₄, filtered and concentrated. The residue was purified by flash column chromatography (silica gel, 0˜20%, ethyl acetate in petroleum ether) to afford tert-butyl (S)-4-(1-(3-fluorophenyl)-3-iodo-1H-pyrrolo[3,2-c]pyridin-4-yl)-3-methylpiperazine-1-carboxylate (250 mg, 36%) as a yellow solid. LC/MS ESI (m/z): 537 (M+H)⁺.

### Step 4. tert-Butyl (S)-4-(1-(3-fluorophenyl)-3-(2-oxoazetidin-1-yl)-1H-pyrrolo[3,2-c]pyridin-4-yl)-3-methylpiperazine-1-carboxylate

To a solution of tert-butyl (S)-4-(1-(3-fluorophenyl)-3-iodo-1H-pyrrolo[3,2-c]pyridin-4-yl)-3-methylpiperazine-1-carboxylate (150 mg, 0.28 mmol) in DMF (5 mL) were added azetidin-2-one (80 mg, 1.1 mmol), CuI (27 mg, 0.14 mmol), trans-N¹,N²-dimethylcyclohexane-1,2-diamine (0.05 mL, 0.28 mmol) and K₃PO₄ (180 mg, 0.84 mmol). The resulting mixture was stirred at 90° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford tert-butyl (S)-4-(1-(3-fluorophenyl)-3-(2-oxoazetidin-1-yl)-1H-pyrrolo[3,2-c]pyridin-4-yl)-3-methylpiperazine-1-carboxylate (120 mg, 89%) as a yellow oil. LC/MS ESI (m/z): 480 (M+H)⁺.

### Step 5. tert-Butyl (S)-4-(3-(azetidin-1-yl)-1-(3-fluorophenyl)-1H-pyrrolo[3,2-c]pyridin-4-yl)-3-methylpiperazine-1-carboxylate

To a solution of tert-butyl (S)-4-(1-(3-fluorophenyl)-3-(2-oxoazetidin-1-yl)-1H-pyrrolo[3,2-c]pyridin-4-yl)-3-methylpiperazine-1-carboxylate (120 mg, 0.25 mmol) in THE (5 mL) at 0° C. were added RhHCO(PPh₃)₃ (23 mg, 0.025 mmol) and PhSiH₃ (0.15 mL, 1.3 mmol). After stirring at room temperature for 1 h under N₂, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford tert-butyl (S)-4-(3-(azetidin-1-yl)-1-(3-fluorophenyl)-1H-pyrrolo[3,2-c]pyridin-4-yl)-3-methylpiperazine-1-carboxylate (80 mg, 68%) as a white solid. LC/MS ESI (m/z): 466 (M+H)⁺. ¹H NMR (400 MHz, CDCl₃) δ 7.93 (d, J=5.9 Hz, 1H), 7.46 (m, 1H), 7.25 (d, J=4.2 Hz, 1H), 7.18 (d, J=9.7 Hz, 1H), 7.08-6.99 (m, 2H), 6.60 (s, 1H), 4.35 (s, 1H), 3.97 (m, 3H), 3.78-3.50 (m, 5H), 3.34 (m, 2H), 2.26 (p, J=7.0 Hz, 2H), 1.49 (s, 9H), 1.03 (d, J=6.1 Hz, 3H).

The following compound was prepared by a procedure analogous to the synthesis of compound 637 from the corresponding chiral amine.

| Cmpd | | |
|---|---|---|
| No. | Chemical Name | LCMS and ¹H NMR |
| 638 | tert-butyl (2R,5S)-4-(3- | LC/MS ESI (m/z): 480 (M + H)⁺. ¹H NMR (400 MHz, |
| | (azetidin-1-yl)-1-(3- | CDCl₃) δ 7.82 (d, J = 6.0 Hz, 1H), 7.42-7.36 (m, 1H), |
| | fluorophenyl)-1H-pyrrolo[3,2- | 7.21-7.17 (m, 1H), 7.14-7.10 (m, 1H), 7.01-6.95 (m, |
| | c]pyridin-4-yl)-2,5- | 1H), 6.90 (d, J = 6.0 Hz, 1H), 6.55 (s, 1H), 4.80 (s, 1H), |
| | dimethylpiperazine-1- | 4.35 (s, 1H), 3.89 (q, J = 6.9 Hz, 2H), 3.76-3.61 (m, 3H), |
| | carboxylate | 3.53 (q, J = 6.8 Hz, 2H), 3.41 (d, J = 13.5 Hz, 1H), 2.25- |
| | | 2.18 (m, 2H), 1.43 (s, 9H), 1.10 (d, J = 6.8 Hz, 3H), 0.91 (d, |
| | | J = 6.7 Hz, 3H). |

### Example 198. Synthesis of tert-butyl (S)-4-(5-cyclobutyl-7-(3-fluorophenyl)-7H-pyrrolo[2,3-d]pyrimidin-4-yl)-3-methylpiperazine-1-carboxylate (Compound 641)

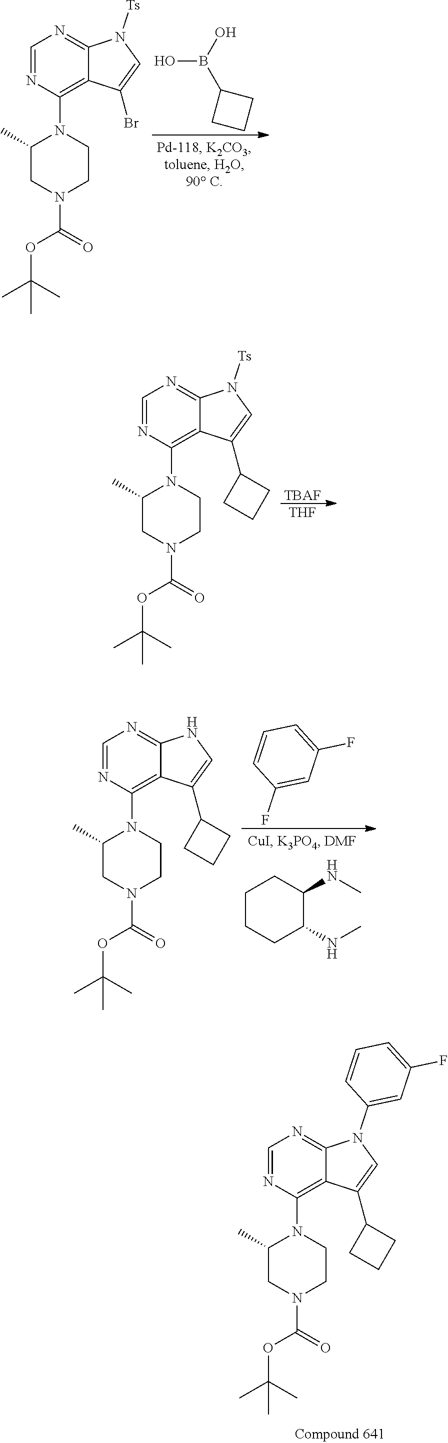

### Step 1. tert-Butyl (S)-4-(5-cyclobutyl-7-tosyl-7H-pyrrolo[2,3-d]pyrimidin-4-yl)-3-methylpiperazine-1-carboxylate

To a solution of tert-butyl (S)-4-(5-bromo-7-tosyl-7H-pyrrolo[2,3-d]pyrimidin-4-yl)-3-methylpiperazine-1-carboxylate (200 mg, 0.36 mmol, prepared following a similar procedure outlined for compound 666) in toluene (10 mL) and H₂O (0.1 mL) were added cyclobutylboronic acid (72 mg, 0.72 mmol), K₂CO₃ (250 mg, 1.8 mmol) and Pd(dptf)Cl₂ (32 mg, 0.04 mmol). The resulting mixture was stirred at 90° C. overnight. After cooling to room temperature, the reaction was concentrated and the residue was purified by flash column chromatography (silica gel, 0˜30%, ethyl acetate in petroleum ether) to afford tert-butyl (S)-4-(5-cyclobutyl-7-tosyl-7H-pyrrolo[2,3-d]pyrimidin-4-yl)-3-methylpiperazine-1-carboxylate (80 mg, 42%) as a solid. LC/MS ESI (m/z): 526 (M+H)⁺.

### Step 2. tert-Butyl (S)-4-(5-cyclobutyl-7H-pyrrolo[2,3-d]pyrimidin-4-yl)-3-methylpiperazine-1-carboxylate

To a solution of tert-butyl (S)-4-(5-cyclobutyl-7-tosyl-7H-pyrrolo[2,3-d]pyrimidin-4-yl)-3-methylpiperazine-1-carboxylate (80 mg, 0.15 mmol) in THE (2 mL) was added TBAF (1.0 mL, 1.0 M in THF). After stirring at room temperature under N₂ for 2 h, the reaction was partitioned between EtOAc and brine. The aqueous layer was extracted twice with EtOAc. The combined organic layers were washed with brine, dried over Na₂SO₄, filtered and concentrated. The residue was used directly in the next step. LC/MS ESI (m/z): 372 (M+H)⁺.

### Step 3. tert-Butyl (S)-4-(5-cyclobutyl-7-(3-fluorophenyl)-7H-pyrrolo[2,3-d]pyrimidin-4-yl)-3-methylpiperazine-1-carboxylate

To a solution of tert-butyl (S)-4-(5-cyclobutyl-7H-pyrrolo[2,3-d]pyrimidin-4-yl)-3-methylpiperazine-1-carboxylate (50 mg, 0.13 mmol) in DMF (3 mL) were added 1-fluoro-3-iodobenzene (45 mg, 0.20 mmol), trans-N, N-dimethylcyclohexane-1,2-diamine (14 mg, 0.10 mmol), CuI (19 mg, 0.10 mmol) and K₃PO₄ (43 mg, 0.20 mmol). The resulting mixture was heated to 100° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0-50% ethyl acetate in petroleum ether) to afford tert-butyl (S)-4-(5-cyclobutyl-7-(3-fluorophenyl)-7H-pyrrolo[2,3-d]pyrimidin-4-yl)-3-methylpiperazine-1-carboxylate (25 mg, 41%) as a white solid. LC/MS ESI (m/z): 466 (M+H)⁺. ¹H NMR (400 MHz, CDCl₃) δ 8.41 (s, 1H), 7.49-7.37 (m, 3H), 7.17 (s, 1H), 6.98 (t, J=8.1 Hz, 1H), 4.17 (s, 1H), 3.94 (s, 1H), 3.76-3.59 (m, 2H), 3.43 (s, 3H), 3.16 (s, 1H), 2.44-2.32 (m, 2H), 2.12-1.86 (m, 4H), 1.43 (s, 9H), 1.12 (d, J=5.5 Hz, 3H).

The following compounds were prepared by procedures analogous to the synthesis of compound 641 from the corresponding chiral amines, boronic acids and aryl halides.

| Cmpd | | |
|---|---|---|
| No. | Chemical Name | LCMS and ¹H NMR |
| 642 | tert-butyl (2R,5S)-4-(5- | LC/MS ESI (m/z): 479 (M + H)⁺. ¹H NMR (400 MHz, |
| | cyclopropyl-7-(4- | CDCl₃) δ 8.47 (s, 1H), 8.41 (d, J = 2.0 Hz, 1H), 8.26 (d, J = |
| | methoxypyridin-2-yl)-7H- | 5.8 Hz, 1H), 7.77 (s, 1H), 6.72 (dd, J = 5.8, 2.3 Hz, 1H), |
| | pyrrolo[2,3-d]pyrimidin-4-yl)- | 4.87 (s, 1H), 4.42 (br. s, 1H), 3.96 (s, 3H), 3.81-3.67 (m, |
| | 2,5-dimethylpiperazine-1- | 3H), 3.58 (d, J = 12.4 Hz, 1H), 2.09-1.96 (m, 1H), 1.50 (s, |
| | carboxylate | 9H), 1.16 (m, 6H), 1.03-0.94 (m, 3H), 0.75-0.62 (m, |
| | | 1H). |
| 643 | tert-butyl (2R,5S)-4-(5- | LC/MS ESI (m/z): 449 (M + H)⁺. ¹H NMR (400 MHz, |
| | cyclopropyl-7-(pyridin-2-yl)- | CDCl₃) δ 8.71 (d, J = 8.4 Hz, 1H), 8.47 (m, 2H), 7.88- |
| | 7H-pyrrolo[2,3-d]pyrimidin-4- | 7.82 (m, 1H), 7.77 (s, 1H), 7.17 (m, 1H), 4.93-4.83 (m, |
| | yl)-2,5-dimethylpiperazine-1- | 1H), 4.42 (br. s, 1H), 3.75 (m, 3H), 3.59 (d, J = 11.4 Hz, |
| | carboxylate | 1H), 2.02 (m, 1H), 1.50 (s, 9H), 1.19 (d, J = 6.8 Hz, 3H), |
| | | 1.14 (d, J = 6.7 Hz, 3H), 1.04-0.94 (m, 3H), 0.74-0.67 |
| | | (m, 1H). |
| 644 | tert-butyl (2R,5S)-4-(5- | LC/MS ESI (m/z): 463 (M + H)⁺. ¹H NMR (400 MHz, |
| | cyclopropyl-7-(4-methylpyridin- | CDCl₃) δ 8.51 (s, 1H), 8.48 (s, 1H), 8.32 (d, J = 5.0 Hz, |
| | 2-yl)-7H-pyrrolo[2,3- | 1H), 7.74 (d, J = 0.8 Hz, 1H), 7.00 (dd, J = 5.0, 0.6 Hz, |
| | d]pyrimidin-4-yl)-2,5- | 1H), 4.87 (s, 1H), 4.42 (s, 1H), 3.74 (m, 3H), 3.58 (d, J = |
| | dimethylpiperazine-1- | 11.7 Hz, 1H), 2.48 (s, 3H), 2.02 (m, 1H), 1.50 (s, 9H), 1.19 |
| | carboxylate | (d, J = 6.8 Hz, 3H), 1.14 (d, J = 6.7 Hz, 3H), 1.03-0.94 |
| | | (m, 3H), 0.69 (m, 1H). |
| 645 | tert-butyl (2R,5S)-4-(5- | LC/MS ESI (m/z): 530 (M + H)⁺. ¹H NMR (400 MHz, |
| | cyclobutyl-7-(3- | CDCl₃) δ 8.43 (s, 1H), 8.00 (d, J = 8.0 Hz, 1H), 7.95 (s, |
| | (trifluoromethyl)phenyl)-7H- | 1H), 7.67-7.59 (m, 2H), 7.24 (s, 1H), 4.55-4.30 (m, 2H), |
| | pyrrolo[2,3-d]pyrimidin-4-yl)- | 3.80-3.65 (m, 4H), 3.47 (d, J = 13.0 Hz, 1H), 2.51-2.41 |
| | 2,5-dimethylpiperazine-1- | (m, 2H), 2.21-1.94 (m, 4H), 1.50 (s, 9H), 1.14 (d, J = 6.7 |
| | carboxylate | Hz, 6H). |
| 646 | tert-butyl (2R,5S)-4-(5- | LC/MS ESI (m/z): 480 (M + H)⁺. ¹H NMR (400 MHz, |
| | cyclobutyl-7-(3-fluorophenyl)- | CDCl₃) δ 8.37 (s, 1H), 7.49-7.36 (m, 3H), 7.15 (s, 1H), |
| | 7H-pyrrolo[2,3-d]pyrimidin-4- | 7.01-6.94 (m, 1H), 4.49-4.19 (m, 2H), 3.73-3.55 (m, |
| | yl)-2,5-dimethylpiperazine-1- | 4H), 3.38 (d, J = 13.2 Hz, 1H), 2.45-2.32 (m, 2H), 2.16- |
| | carboxylate | 1.85 (m, 4H), 1.43 (s, 9H), 1.10-1.03 (m, 6H). |
| 647 | tert-butyl (2R,5S)-4-(7-(3- | LC/MS ESI (m/z): 496 (M + H)⁺. ¹H NMR(400 MHz, |
| | chlorophenyl)-5-cyclobutyl-7H- | CDCl₃) δ 8.43 (s, 1H), 7.74 (t, J = 2.0 Hz, 1H), 7.66 (dd, J = |
| | pyrrolo[2,3-d]pyrimidin-4-yl)- | 8.1, 1.0 Hz, 1H), 7.44 (t, J = 8.1 Hz, 1H), 7.32 (dd, J = |
| | 2,5-dimethylpiperazine-1- | 8.0, 0.9 Hz, 1H), 7.21 (s, 1H), 4.50-4.30 (m, 2H), 3.79- |
| | carboxylate | 3.64 (m, 4H), 3.45 (d, J = 13.2 Hz, 1H), 2.49-2.40 (m, |
| | | 2H), 2.20-1.94 (m, 4H), 1.50 (s, 9H), 1.13 (m, 6H). |
| 648 | tert-butyl (2R,5S)-4-(5- | LC/MS ESI (m/z): 498 (M + H)⁺. ¹H NMR(400 MHz, |
| | cyclobutyl-7-(3,5- | CDCl₃) δ 8.44 (s, 1H), 7.41 (d, J = 6.5 Hz, 2H), 7.20 (s, |
| | difluorophenyl)-7H-pyrrolo[2,3- | 1H), 6.78 (t, J = 8.6 Hz, 1H), 4.50-4.30 (m, 2H), 3.79- |
| | d]pyrimidin-4-yl)-2,5- | 3.63 (m, 4H), 3.45 (d, J = 13.1 Hz, 1H), 2.51-2.40 (m, |
| | dimethylpiperazine-1- | 2H), 2.19-1.94 (m, 4H), 1.50 (s, 9H), 1.13 (m, 6H). |
| | carboxylate | |

### Example 199. Synthesis of tert-butyl (2R,5S)-4-(5-(2-fluorophenyl)-7-(4-(trifluoromethyl)pyridin-2-yl)-7H-pyrrolo[2,3-d]pyrimidin-4-yl)-2,5-dimethylpiperazine-1-carboxylate (Compound 649)

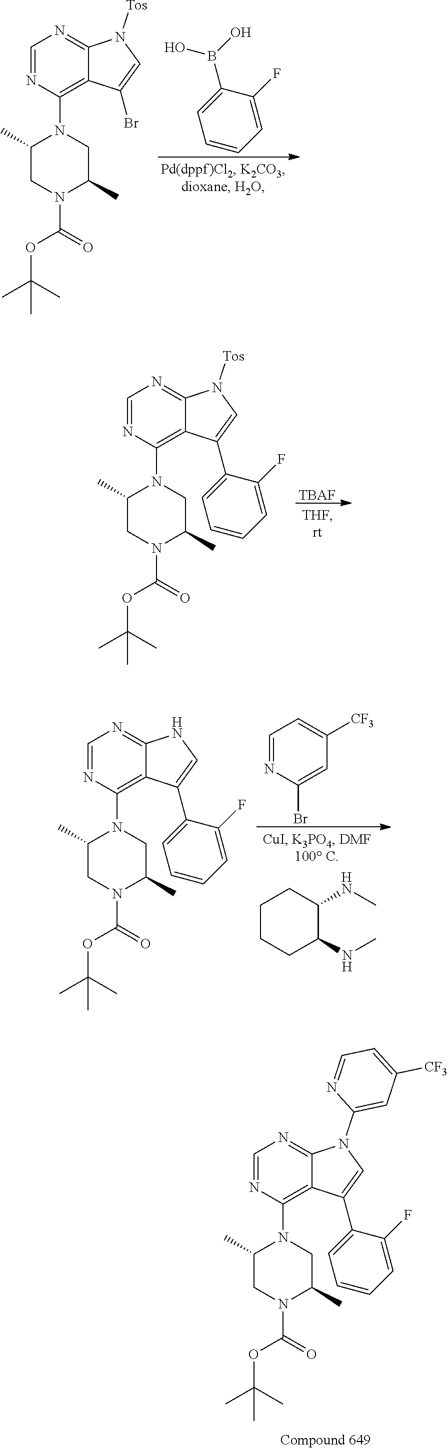

### Step 1. tert-Butyl (2R,5S)-4-(5-(2-fluorophenyl)-7-tosyl-7H-pyrrolo[2,3-d]pyrimidin-4-yl)-2,5-dimethylpiperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (6.0 g, 9.8 mmol) in dioxane (60 mL) and H₂O (10 mL) were added (2-fluorophenyl)boronic acid (1.7 g, 12 mmol), K₂CO₃ (4.1 g, 29 mmol) and Pd(dppf)Cl₂ (0.72 g, 0.98 mmol). The resulting mixture was stirred at 90° C. overnight. After cooling to room temperature, the reaction was concentrated and the residue was purified by flash column chromatography (silica gel, 0˜30%, ethyl acetate in petroleum ether) to afford tert-butyl (2R,5S)-4-(5-(2-fluorophenyl)-7-tosyl-7H-pyrrolo[2,3-d]pyrimidin-4-yl)-2,5-dimethylpiperazine-1-carboxylate (5.5 g, 96%) as a yellow solid. LC/MS ESI (m/z): 580 (M+H)⁺.

### Step 2. tert-Butyl (2R,5S)-4-(5-(2-fluorophenyl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7-tosyl-7H-pyrrolo[2,3-d]pyrimidin-4-yl)-2,5-dimethylpiperazine-1-carboxylate (500 mg, 0.86 mmol) in THE (5 mL) was added TBAF (5.0 mL, 1.0 M in THF). After stirring at room temperature overnight, the reaction was diluted with water and extracted twice with EtOAc. The combined organic layers were washed four times with brine, dried over Na₂SO₄, filtered, and concentrated to afford tert-butyl (2R,5S)-4-(5-(2-fluorophenyl)-7H-pyrrolo[2,3-d]pyrimidin-4-yl)-2,5-dimethylpiperazine-1-carboxylate (350 mg, 95%) as a solid. LC/MS ESI (m/z): 426 (M+H)⁺.

### Step 3. tert-Butyl (2R,5S)-4-(5-(2-fluorophenyl)-7-(4-(trifluoromethyl)pyridin-2-yl)-7H-pyrrolo[2,3-d]pyrimidin-4-yl)-2,5-dimethylpiperazine-1-carboxylate

To a solution of tert-butyl (2R,5S)-4-(5-(2-fluorophenyl)-7H-pyrrolo[2,3-d]pyrimidin-4-yl)-2,5-dimethylpiperazine-1-carboxylate (300 mg, 0.71 mmol) in DMF (10 mL) were added trans-N¹,N²-dimethylcyclohexane-1,2-diamine (100 mg, 0.71 mmol), 2-bromo-4-(trifluoromethyl)pyridine (400 mg, 1.8 mmol), CuI (140 mg, 0.71 mmol) and K₃PO₄ (450 mg, 2.1 mmol). The resulting mixture was heated to 100° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜35% ethyl acetate in petroleum ether) to afford tert-butyl (2R,5S)-4-(5-(2-fluorophenyl)-7-(4-(trifluoromethyl)pyridin-2-yl)-7H-pyrrolo[2,3-d]pyrimidin-4-yl)-2,5-dimethylpiperazine-1-carboxylate (350 mg, 86%) as a solid. 50 mg of which was further purified by prep-HPLC to afford 30 mg of a white solid. LC/MS ESI (m/z): 571 (M+H)⁺. ¹H NMR (400 MHz, CDCl₃) δ 9.27 (s, 1H), 8.57 (d, J=5.1 Hz, 1H), 8.51 (s, 1H), 8.25 (s, 1H), 7.43 (td, J=7.6, 1.8 Hz, 1H), 7.37-7.27 (m, 2H), 7.19-7.11 (m, 2H), 4.30-3.95 (m, 2H), 3.33-3.13 (m, 3H), 2.93-2.64 (m, 1H), 1.36 (s, 9H), 0.99 (d, J=6.8 Hz, 3H), 0.89 (d, J=6.7 Hz, 3H).

The following compounds were prepared by procedures analogous to the synthesis of compound 649 from the corresponding aryl halides and amines.

| Cmpd | | |
|---|---|---|
| No. | Chemical Name | LCMS and ¹H NMR |
| 650 | tert-butyl (2R,5S)-4-(7-(5- | LC/MS ESI (m/z): 528 (M + H)⁺. ¹H NMR (400 MHz, |
| | cyanopyridin-3-yl)-5-(2- | CDCl₃) δ 9.16 (d, J = 2.5 Hz, 1H), 8.77 (d, J = 1.7 Hz, 1H), |
| | fluorophenyl)-7H-pyrrolo[2,3- | 8.65 (t, J = 2.1 Hz, 1H), 8.44 (s, 1H), 7.44-7.37 (m, 2H), |
| | d]pyrimidin-4-yl)-2,5- | 7.37-7.30 (m, 1H), 7.23-7.20 (m, 1H), 7.18-7.13 (m, |
| | dimethylpiperazine-1- | 1H), 4.27-3.98 (m, 2H), 3.34-3.11 (m, 3H), 3.01-2.68 |
| | carboxylate | (m, 1H), 1.36 (s, 9H), 0.99 (d, J = 6.8 Hz, 3H), 0.93 (d, J = |
| | | 6.6 Hz, 3H). |
| 651 | tert-butyl (S)-4-(5-(2- | LC/MS ESI (m/z): 557 (M + H)⁺. ¹H NMR (400 MHz, |
| | fluorophenyl)-7-(4- | CDCl₃) δ 9.26 (s, 1H), 8.56 (d, J = 5.1 Hz, 1H), 8.51 (s, |
| | (trifluoromethyl)pyridin-2-yl)- | 1H), 8.23 (s, 1H), 7.44-7.38 (m, 1H), 7.34 (d, J = 4.6 Hz, |
| | 7H-pyrrolo[2,3-d]pyrimidin-4- | 1H), 7.32-7.26 (m, 1H), 7.14 (m, 2H), 4.13 (m, 1H), 3.71 |
| | yl)-3-methylpiperazine-1- | (m, 1H), 3.47-3.35 (m, 2H), 3.07-2.96 (m, 1H), 2.77 (m, |
| | carboxylate | 2H), 1.36 (s, 9H), 0.93 (d, J = 4.6 Hz, 3H). |

### Example 200. Synthesis of 1-methylcyclopropyl (2R,5S)-4-(5-(2-fluorophenyl)-7-(4-(trifluoromethyl)pyridin-2-yl)-7H-pyrrolo[2,3-d]pyrimidin-4-yl)-2,5-dimethylpiperazine-1-carboxylate (Compound 652)

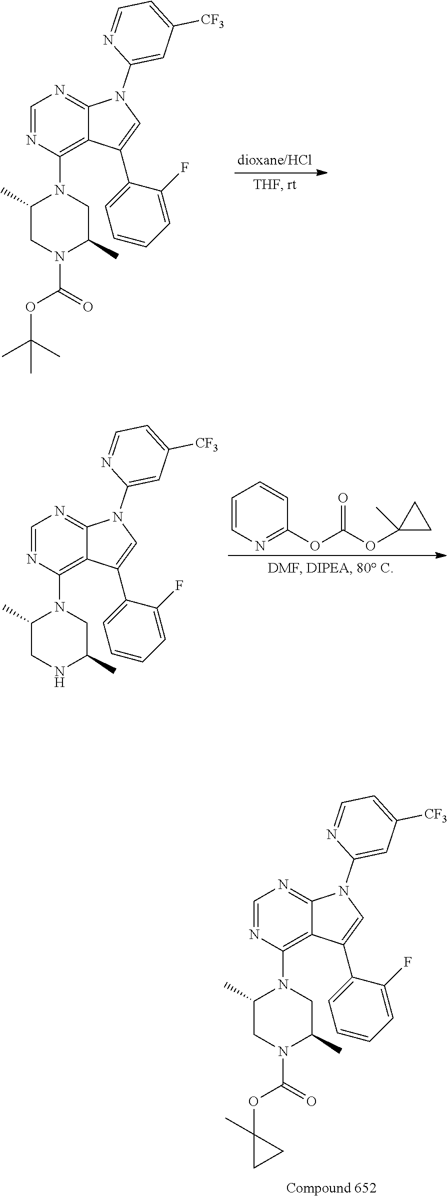 [SMILES: C[C@@H]1CN(c2ncnc3c2c(-c2ccccc2F)cn3-c2cc(C(F)(F)F)ccn2)[C@@H](C)CN1C(=O)OC(C)(C)C ; C[C@@H]1CN(c2ncnc3c2c(-c2ccccc2F)cn3-c2cc(C(F)(F)F)ccn2)[C@@H](C)CN1 ; CC1(OC(=O)Oc2ccccn2)CC1 ; C[C@@H]1CN(c2ncnc3c2c(-c2ccccc2F)cn3-c2cc(C(F)(F)F)ccn2)[C@@H](C)CN1C(=O)OC1(C)CC1]

### Step 1. 4-((2S,5R)-2,5-Dimethylpiperazin-1-yl)-5-(2-fluorophenyl)-7-(4-(trifluoromethyl)pyridin-2-yl)-7H-pyrrolo[2,3-d]pyrimidine

To a solution of tert-butyl (2R,5S)-4-(5-(2-fluorophenyl)-7-(4-(trifluoromethyl)pyridin-2-yl)-7H-pyrrolo[2,3-d]pyrimidin-4-yl)-2,5-dimethylpiperazine-1-carboxylate (100 mg, 0.18 mmol, prepared following the procedure outlined for compound 649) in DCM (4 mL) was added HCl (0.8 mL, 4.0 M in dioxane). After stirring at room temperature for 3 h, the reaction mixture was basified with NaHCO₃ (aq.) to pH 8 and extracted twice with DCM. The combined organic layers were dried over Na₂SO₄, filtered and concentrated. The residue was purified by flash column chromatography (0˜10% MeOH in DCM) to afford 4-((2S,5R)-2,5-dimethylpiperazin-1-yl)-5-(2-fluorophenyl)-7-(4-(trifluoromethyl)pyridin-2-yl)-7H-pyrrolo[2,3-d]pyrimidine (60 mg, 73%) as a white solid. LC/MS ESI (m/z): 471 (M+H)⁺.

### Step 2. 1-Methylcyclopropyl (2R,5S)-4-(5-(2-fluorophenyl)-7-(4-(trifluoromethyl)pyridin-2-yl)-7H-pyrrolo[2,3-d]pyrimidin-4-yl)-2,5-dimethylpiperazine-1-carboxylate

To a solution of 2-(4-((2R,5S)-2,5-dimethylpiperazin-1-yl)-5-(trifluoromethyl)-7H-pyrrolo[2,3-d]pyrimidin-7-yl)isonicotinonitrile (50 mg, 0.11 mmol) in DMF (5 mL) were added 1-methylcyclopropyl pyridin-2-yl carbonate (3.0 eq., prepared following a similar procedure outlined for compound 623) and DIPEA (43 mg, 0.33 mmol). The resulting mixture was heated to 80° C. for 3 h. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜80%, ethyl acetate in petroleum ether) to give crude product, which was further purified by prep-HPLC to afford 1-methylcyclopropyl (2R,5S)-4-(5-(2-fluorophenyl)-7-(4-(trifluoromethyl)pyridin-2-yl)-7H-pyrrolo[2,3-d]pyrimidin-4-yl)-2,5-dimethylpiperazine-1-carboxylate (10 mg, 17%) as a white solid. LC/MS ESI (m/z): 569 (M+H)⁺. ¹H NMR (400 MHz, CDCl₃) δ 9.26 (s, 1H), 8.57 (d, J=5.1 Hz, 1H), 8.51 (s, 1H), 8.25 (s, 1H), 7.46-7.37 (m, 1H), 7.37-7.26 (m, 2H), 7.19-7.10 (m, 2H), 4.30-3.92 (m, 2H), 3.35-3.11 (m, 3H), 2.94-2.61 (m, 1H), 1.44 (s, 3H), 1.00 (d, J=6.8 Hz, 3H), 0.87 (d, J=6.7 Hz, 3H), 0.79-0.71 (m, 2H), 0.59-0.48 (m, 2H).

The following compound was prepared by a procedure analogous to the synthesis of compound 652 from the corresponding aryl halide.

| Cmpd | | |
|---|---|---|
| No | Chemical Name | LCMS and ¹H NMR |
| 653 | 1-methylcyclopropyl (2R,5S)-4- | LC/MS ESI (m/z): 519 (M + H)⁺. ¹H NMR(400 MHz, |
| | (5-(2-fluorophenyl)-7-(4- | CDCl₃) δ 8.74 (dd, J = 10.9, 2.2 Hz, 1H), 8.49 (s, 1H), 8.36 |
| | fluoropyridin-2-yl)-7H- | (dd, J = 8.7, 5.7 Hz, 1H), 8.23 (s, 1H), 7.44-7.39 (m, 1H), |
| | pyrrolo[2,3-d]pyrimidin-4-yl)- | 7.33-7.27 (m, 1H), 7.19-7.10 (m, 2H), 6.91-6.87 (m, |
| | 2,5-dimethylpiperazine-1- | 1H), 4.27-3.96 (m, 2H), 3.32-3.13 (m, 3H), 2.85-2.64 |
| | carboxylate | (m, 1H), 1.44 (s, 3H), 1.00 (d, J = 6.8 Hz, 3H), 0.86 (d, J = |
| | | 6.6 Hz, 3H), 0.78-0.71 (m, 2H), 0.57-0.49 (m, 2H). |

### Example 201. Synthesis of 1-methylcyclopropyl (2R,5S)-4-(7-(3-chlorophenyl)-5-cyclopropyl-7H-pyrrolo[2,3-d]pyrimidin-4-yl)-2,5-dimethylpiperazine-1-carboxylate (Compound 654)

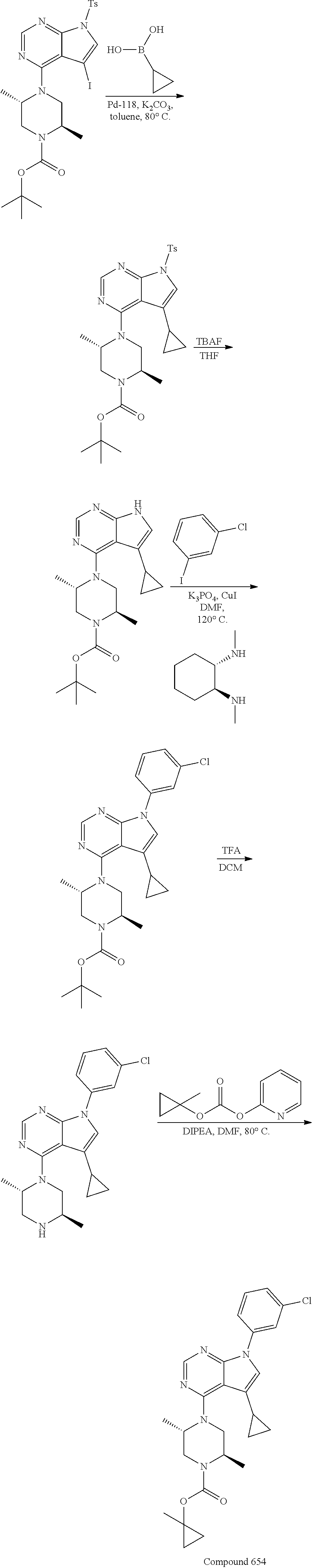

### Step 1. tert-Butyl (2R,5S)-4-(5-cyclopropyl-7-tosyl-7H-pyrrolo[2,3-d]pyrimidin-4-yl)-2,5-dimethylpiperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (2.0 g, 3.3 mmol, prepared following the procedure outlined for compound 666) in toluene (25 mL) were added cyclopropylboronic acid (570 mg, 6.6 mmol), K₂CO₃ (6.8 g, 50 mmol) and PdCl₂(dtbpf) (190 mg, 0.30 mmol). The resulting mixture was heated to 80° C. overnight. After cooling to room temperature, solvent was removed and the residue was purified by flash column chromatography (silica gel, 0-40% ethyl acetate in petroleum ether) to afford tert-butyl (2R,5S)-4-(5-cyclopropyl-7-tosyl-7H-pyrrolo[2,3-d]pyrimidin-4-yl)-2,5-dimethylpiperazine-1-carboxylate (1.1 g, 65%) as a white solid. LC/MS ESI (m/z): 526 (M+H)⁺.

### Step 2. tert-Butyl (2R,5S)-4-(5-cyclopropyl-7H-pyrrolo[2,3-d]pyrimidin-4-yl)-2,5-dimethylpiperazine-1-carboxylate

To a solution of tert-butyl (2R,5S)-4-(5-cyclopropyl-7-tosyl-7H-pyrrolo[2,3-d]pyrimidin-4-yl)-2,5-dimethylpiperazine-1-carboxylate (1.1 g, 2.1 mmol) in THE (20 mL) was added TBAF (11 mL, 1.0 M in THF). After stirring at room temperature for 5 h, the reaction was diluted with water and extracted twice with EtOAc. The combined organic layers were washed with brine, dried over Na₂SO₄, filtered and concentrated. The residue was purified by flash column chromatography (silica gel, 0˜70% ethyl acetate in petroleum ether) to afford tert-butyl (2R,5S)-4-(5-cyclopropyl-7H-pyrrolo[2,3-d]pyrimidin-4-yl)-2,5-dimethylpiperazine-1-carboxylate (600 mg, 77%) as a white solid. LC/MS ESI (m/z): 372 (M+H)⁺.

### Step 3. tert-Butyl (2R,5S)-4-(7-(3-chlorophenyl)-5-cyclopropyl-7H-pyrrolo[2,3-d]pyrimidin-4-yl)-2,5-dimethylpiperazine-1-carboxylate

To a solution of tert-butyl (2R,5S)-4-(5-cyclopropyl-7H-pyrrolo[2,3-d]pyrimidin-4-yl)-2,5-dimethylpiperazine-1-carboxylate (300 mg, 0.80 mmol) in DMF (5 mL) were added 1-chloro-3-iodobenzene (360 mg, 1.5 mmol), trans-N,N′-dimethylcyclohexane-1,2-diamine (57 mg, 0.40 mmol), CuI (76 mg, 0.40 mmol) and K₃PO₄ (490 mg, 2.3 mmol). The resulting mixture was heated to 120° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0-30% ethyl acetate in petroleum ether) to afford tert-butyl (2R,5S)-4-(7-(3-chlorophenyl)-5-cyclopropyl-7H-pyrrolo[2,3-d]pyrimidin-4-yl)-2,5-dimethylpiperazine-1-carboxylate (330 mg, 85%) as a white solid. LC/MS ESI (m/z): 482 (M+H)⁺.

### Step 4. 7-(3-Chlorophenyl)-5-cyclopropyl-4-((2S,5R)-2,5-dimethylpiperazin-1-yl)-7H-pyrrolo[2,3-d]pyrimidine

To a solution of tert-butyl (2R,5S)-4-(7-(3-chlorophenyl)-5-cyclopropyl-7H-pyrrolo[2,3-d]pyrimidin-4-yl)-2,5-dimethylpiperazine-1-carboxylate (330 mg, 0.69 mmol) in DCM (5 m1) was added TFA (2.0 m1). The resulting mixture was stirred at room temperature overnight. After removal of solvents, the residue was diluted with DCM, and washed with NaHCO₃ (aq.). The aqueous layer was extracted twice with DCM. The combined organic layers were washed with brine, dried over Na₂SO₄, filtered and concentrated. The residue was purified by flash column chromatography (silica gel, 0-80% ethyl acetate in petroleum ether) to afford 7-(3-chlorophenyl)-5-cyclopropyl-4-((2S,5R)-2,5-dimethylpiperazin-1-yl)-7H-pyrrolo[2,3-d]pyrimidine (200 mg, 77%) as a white solid. LC/MS ESI (m/z): 382 (M+H)⁺.

### Step 5. 1-Methylcyclopropyl (2R,5S)-4-(7-(3-chlorophenyl)-5-cyclopropyl-7H-pyrrolo[2,3-d]pyrimidin-4-yl)-2,5-dimethylpiperazine-1-carboxylate

To a solution of 7-(3-chlorophenyl)-5-cyclopropyl-4-((2S,5R)-2,5-dimethylpiperazin-1-yl)-7H-pyrrolo[2,3-d]pyrimidine (100 mg, 0.26 mmol) in DMF (10 mL) were added 1-methylcyclopropyl pyridin-4-yl carbonate (3.0 eq., prepared following a similar procedure outlined for compound 623) and DIEA (100 mg, 0.78 mmol). After stirring at 80° C. overnight, the reaction was diluted with H₂O (50 mL) and extracted twice with EtOAc. The combined organic layers were dried over Na₂SO₄, filtered and concentrated. The residue was purified by flash column chromatography (silica gel, 0˜30% ethyl acetate in petroleum ether) to give crude product which was further purified by prep-HPLC to afford 1-methylcyclopropyl (2R,5S)-4-(7-(3-chlorophenyl)-5-cyclopropyl-7H-pyrrolo[2,3-d]pyrimidin-4-yl)-2,5-dimethylpiperazine-1-carboxylate (14 mg, 11%) as a white solid. LC/MS ESI (m/z): 480 (M+H)⁺. ¹H NMR (400 MHz, CD₃OD) δ 8.28 (s, 1H), 7.82 (s, 1H), 7.62 (d, J=8.1 Hz, 1H), 7.49 (t, J=7.3 Hz, 1H), 7.37 (d, J=8.0 Hz, 1H), 7.26 (s, 1H), 4.99-4.90 (m, 1H), 4.43 (s, 1H), 3.79 (s, 3H), 3.65 (m, 1H), 2.05 (s, 1H), 1.55 (s, 3H), 1.20-1.11 (m, 6H), 1.04 (d, J=8.0 Hz, 2H), 0.97-0.86 (m, 3H), 0.67 (m, 3H).

The following compounds were prepared by a procedure similar to the synthesis of compound 654 from the corresponding aryl halide.

| Cmpd | | |
|---|---|---|
| No. | Chemical Name | LCMS and ¹H NMR |
| 655 | 1-methylcyclopropyl (2R,5S)-4- | LC/MS ESI (m/z): 482 (M + H)⁺. ¹H NMR (400 MHz, |
| | (5-cyclopropyl-7-(3,5- | CDCl₃) δ 8.44 (s, 1H), 7.35 (d, J = 7.4 Hz, 2H), 7.26 (s, |
| | difluorophenyl)-7H-pyrrolo[2,3- | 1H), 6.77 (t, J = 8.5 Hz, 1H), 4.90 (s, 1H), 4.40 (m, 1H), |
| | d\|pyrimidin-4-yl)-2,5- | 3.75 (s, 3H), 3.58 (d, J = 11.3 Hz, 1H), 2.00 (s, 1H), 1.58 (S, |
| | dimethylpiperazine-1- | 3H), 1.20-1.12 (m, 6H), 1.03 (d, J = 8.0 Hz, 2H), 0.88 (m, |
| | carboxylate | 3H), 0.65 (m, 3H). |
| 656 | tert-butyl (R)-4-(5-cyclopropyl- | LC/MS ESI (m/z): 452 (M + H)⁺. ¹H NMR (400 MHz, |
| | 7-(3-fluorophenyl)-7H- | CD₃OD) δ 8.29-8.24 (m, 1H), 7.61-7.54 (m, 1H), 7.52- |
| | pyrrolo[2,3-d]pyrimidin-4-yl)-2- | 7.45 (m, 2H), 7.27-7.21 (m, 1H), 7.13-7.03 (m, 1H), |
| | methylpiperazine-1-carboxylate | 4.54-4.45 (m, 1H), 4.40 (s, 1H), 4.15-4.06 (m, 1H), 3.97- |
| | | 3.89 (m, 1H), 3.49-3.32 (m, 2H), 3.13-3.02 (m, 1H), |
| | | 2.11-2.00 (m, 1H), 1.48 (s, 9H), 1.23-1.17 (m, 3H), 1.07- |
| | | 0.99 (m, 2H), 0.95-0.86 (m, 1H), 0.72-0.64 (m, 1H). |
| 657 | tert-butyl (2R,5S)-4-(5- | LC/MS ESI (m/z): 466 (M + H)⁺. ¹HN MR(400 MHz, |
| | cyclopropyl-7-(3-fluorophenyl)- | CDCl₃) δ 8.36 (s, 1H), 7.43-7.36 (m, 3H), 6.98-6.93 (m, |
| | 7H-pyrrolo[2,3-d]pyrimidin-4- | 1H), 6.87 (s, 1H), 4.88-4.80 (m, 1H), 4.49-4.23 (m, 1H), |
| | yl)-2,5-dimethylpiperazine-1- | 3.75-3.66 (m, 3H), 3.56-3.47 (m, 1H), 1.99-1.93 (m, |
| | carboxylate | 1H), 1.43 (s, 9H), 1.10 (m, 6H), 0.98-0.93 (m, 2H), 0.81- |
| | | 0.77 (m, 1H), 0.61-0.55 (m, 1H). |
| 658 | 1-methylcyclopropyl (2R,5S)-4- | LC/MS ESI (m/z): 464 (M + H)⁺. ¹H NMR (400 MHz, |
| | (5-cyclopropyl-7-(3- | CD₃OD) δ 8.29 (s, 1H), 7.62-7.57 (m, 1H), 7.54-7.49 |
| | fluorophenyl)-7H-pyrrolo[2,3- | (m, 2H), 7.27 (s, 1H), 7.14-7.07 (m, 1H), 4.94 (s, 1H), |
| | d]pyrimidin-4-yl)-2,5- | 4.49-4.27 (m, 1H), 3.78 (m, 3H), 3.65 (d, J = 11.5 Hz, |
| | dimethylpiperazine-1- | 1H), 2.08-2.02 (m, 1H), 1.55 (s, 3H), 1.18 (d, J = 6.8 Hz, |
| | carboxylate | 3H), 1.13 (d, J = 6.6 Hz, 3H), 1.08-1.02 (m, 2H), 0.96- |
| | | 0.86 (m, 3H), 0.70-0.64 (m, 3H). |

### Example 202. Synthesis of 1-methylcyclopropyl (2R,5S)-4-(7-(4-fluoropyridin-2-yl)-5-(trifluoromethyl)-7H-pyrrolo[2,3-d]pyrimidin-4-yl)-2,5-dimethylpiperazine-1-carboxylate (Compound 659)

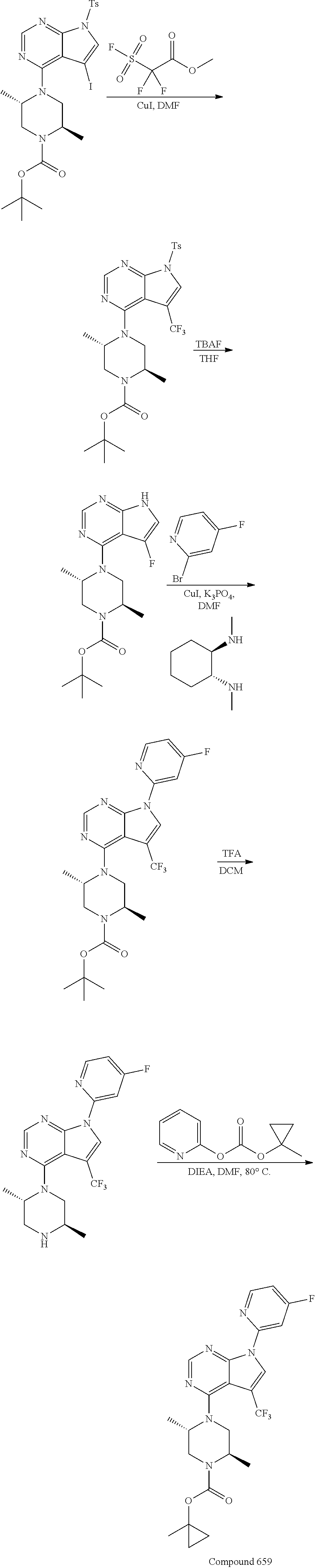

### Step 1. tert-Butyl (2R,5S)-2,5-dimethyl-4-(7-tosyl-5-(trifluoromethyl)-7H-pyrrolo[2,3-d]pyrimidin-4-yl)piperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (26 g, 43 mmol, prepared following the procedure outlined for compound 666) in DMF (50 mL) were added methyl 2,2-difluoro-2-(fluorosulfonyl)acetate (24 g, 130 mmol) and CuI (8.0 g, 43 mmol). The resulting mixture was heated to 80° C. overnight. After cooling to room temperature, the reaction was partitioned between EtOAc and water. The aqueous layer was extracted twice with EtOAc and the combined organic layers were washed with brine, dried over Na₂SO₄, filtered and concentrated. The residue was purified by flash column chromatography (silica gel, 0-30%, ethyl acetate in petroleum ether) to afford tert-butyl (2R,5S)-2,5-dimethyl-4-(7-tosyl-5-(trifluoromethyl)-7H-pyrrolo[2,3-d]pyrimidin-4-yl)piperazine-1-carboxylate (13 g, 55%) as a yellow oil. LC/MS ESI (m/z): 554 (M+H)⁺.

### Step 2. tert-Butyl (2R,5S)-2,5-dimethyl-4-(5-(trifluoromethyl)-7H-pyrrolo[2,3-d]pyrimidin-4-yl)piperazine-1-carboxylate

To a solution of tert-butyl (2R,5S)-2,5-dimethyl-4-(7-tosyl-5-(trifluoromethyl)-7H-pyrrolo[2,3-d]pyrimidin-4-yl)piperazine-1-carboxylate (12 g, 22 mmol) in THE (10 mL) was added TBAF (42 mL, 1.0 M in THF). After stirring at room temperature for 2 h, the reaction was diluted with water and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford tert-butyl (2R,5S)-2,5-dimethyl-4-(5-(trifluoromethyl)-7H-pyrrolo[2,3-d]pyrimidin-4-yl)piperazine-1-carboxylate (7.5 g, 87%) as a yellow oil. LC/MS ESI (m/z): 400 (M+H)⁺.

### Step 3. tert-Butyl (2R,5S)-4-(7-(4-fluoropyridin-2-yl)-5-(trifluoromethyl)-7H-pyrrolo[2,3-d]pyrimidin-4-yl)-2,5-dimethylpiperazine-1-carboxylate

To a solution of tert-butyl (2R,5S)-2,5-dimethyl-4-(5-(trifluoromethyl)-7H-pyrrolo[2,3-d]pyrimidin-4-yl)piperazine-1-carboxylate (300 mg, 0.75 mmol) in DMF (10 mL) were added 2-bromo-4-fluoropyridine (160 mg, 0.90 mmol), CuI (70 mg, 0.37 mmol), trans-N¹,N²-dimethylcyclohexane-1,2-diamine (110 mg, 0.75 mmol) and K₃PO₄ (480 mg, 2.3 mmol). The resulting mixture was stirred at 120° C. overnight. After cooling to room temperature, the reaction was partitioned between EtOAc and water and the aqueous layer was extracted twice with EtOAc. The combined organic layers were washed with brine, dried over Na₂SO₄, filtered and concentrated. The residue was purified by flash column chromatography (silica gel, 0˜30% ethyl acetate in petroleum ether) to afford tert-butyl (2R,5S)-4-(7-(4-fluoropyridin-2-yl)-5-(trifluoromethyl)-7H-pyrrolo[2,3-d]pyrimidin-4-yl)-2,5-dimethylpiperazine-1-carboxylate (85 mg, 23%) as a colorless oil. LC/MS ESI (m/z): 495 (M+H)⁺.

### Step 4. 4-((2S,5R)-2,5-Dimethylpiperazin-1-yl)-7-(4-fluoropyridin-2-yl)-5-(trifluoromethyl)-7H-pyrrolo[2,3-d]pyrimidine

To a solution of tert-butyl (2R,5S)-4-(7-(4-fluoropyridin-2-yl)-5-(trifluoromethyl)-7H-pyrrolo[2,3-d]pyrimidin-4-yl)-2,5-dimethylpiperazine-1-carboxylate (85 mg, 0.17 mmol) in DCM (2 mL) was added TFA (1.0 mL). The resulting mixture was stirred at room temperature for 2 h. After removal of solvent, the residue was diluted with DCM, washed with NaHCO₃ (aq.). The organic layer was washed with brine, dried over Na₂SO₄, filtered and concentrated. The residue was used in the next step directly. LC/MS ESI (m/z): 395 (M+H)⁺.

### Step 5. 1-Methylcyclopropyl (2R,5S)-4-(7-(4-fluoropyridin-2-yl)-5-(trifluoromethyl)-7H-pyrrolo[2,3-d]pyrimidin-4-yl)-2,5-dimethylpiperazine-1-carboxylate

To a solution of 4-((2S,5R)-2,5-dimethylpiperazin-1-yl)-7-(4-fluoropyridin-2-yl)-5-(trifluoromethyl)-7H-pyrrolo[2,3-d]pyrimidine (67 mg, 0.17 mmol) in DMF (2 mL) were added 1-methylcyclopropyl pyridin-2-yl carbonate (3.0 eq., prepared following a similar procedure outlined for compound 623) and DIPEA (0.30 mL). The resulting mixture was heated to 80° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜20% ethyl acetate in petroleum ether) to give crude product which was further purified by prep-HPLC to afford 1-methylcyclopropyl (2R,5S)-4-(7-(4-fluoropyridin-2-yl)-5-(trifluoromethyl)-7H-pyrrolo[2,3-d]pyrimidin-4-yl)-2,5-dimethylpiperazine-1-carboxylate (15 mg, 18%) as a white solid. LC/MS ESI (m/z): 493 (M+H)⁺. ¹H NMR (400 MHz, CD₃OD) δ 8.78-8.66 (m, 2H), 8.62-8.48 (m, 2H), 7.27-7.17 (m, 1H), 4.57 (s, 1H), 4.34 (s, 1H), 3.82-3.63 (m, 3H), 3.49 (d, J=13.1 Hz, 1H), 1.55 (s, 3H), 1.15-1.08 (m, 6H), 0.89 (m, 2H), 0.71-0.59 (m, 2H).

The following compounds were prepared by procedures analogous to the synthesis of compound 659 from the corresponding aryl halides.

| Cmpd | | |
|---|---|---|
| No. | Chemical Name | LCMS and ¹H NMR |
| 660 | 1-methylcyclopropyl (S)-4-(7-(3- | LC/MS ESI (m/z): 494 (M + H)⁺. ¹H NMR (400 MHz, |
| | chlorophenyl)-5- | CD₃OD) δ 8.50 (s, 1H), 8.23 (s, 1H), 7.88 (s, 1H), 7.68 (d, J = |
| | (trifluoromethyl)-7H- | 8.8 Hz, 1H), 7.56 (t, J = 8.0 Hz, 1H), 7.52-7.46 (m, 1H), |
| | pyrrolo[2,3-d]pyrimidin-4-yl)-3- | 4.32 (s, 1H), 3.88 (m, 1H), 3.65 (s, 1H), 3.49 (m, 3H), 3.29- |
| | methylpiperazine-1-carboxylate | 3.17 (m, 1H), 1.55 (s, 3H), 1.15 (d, J = 6.5 Hz, 3H), 0.93- |
| | | 0.86 (m, 2H), 0.67 (m, 2H). |
| 661 | 1-methylcyclopropyl (2R,5S)-4- | LC/MS ESI (m/z): 510 (M + H)⁺. ¹H NMR (400 MHz, |
| | (7-(3,5-difluorophenyl)-5- | CD₃OD) δ 8.47 (s, 1H), 8.28 (s, 1H), 7.61-7.55 (m, 2H), |
| | (trifluoromethyl)-7H- | 7.13-7.05 (m, 1H), 4.59 (s, 1H), 4.34 (s, 1H), 3.81-3.63 |
| | pyrrolo[2,3-d]pyrimidin-4-yl)- | (m, 3H), 3.51 (d, J = 13.8 Hz, 1H), 1.55 (s, 3H), 1.14-1.09 |
| | 2,5-dimethylpiperazine-1- | (m, 6H), 0.89 (s, 2H), 0.71-0.63 (m, 2H). |
| | carboxylate | |
| 662 | tert-butyl (2R,5S)-4-(7-(5- | LC/MS ESI (m/z): 502 (M + H)⁺. ¹H NMR (400 MHz, |
| | cyanopyridin-3-yl)-5- | CDCl₃) δ 9.08 (d, J = 2.5 Hz, 1H), 8.85 (d, J = 1.7 Hz, 1H), |
| | (trifluoromethyl)-7H- | 8.55-8.49 (m, 1H), 8.43 (s, 1H), 7.71 (s, 1H), 4.61-4.52 |
| | pyrrolo[2,3-d]pyrimidin-4-yl)- | (m, 1H), 4.46-4.15 (m, 1H), 3.76-3.64 (m, 2H), 3.60- |
| | 2,5-dimethylpiperazine-1- | 3.53 (m, 1H), 3.48-3.42 (m, 1H), 1.43 (s, 9H), 1.11 (d, J = |
| | carboxylate | 6.6 Hz, 3H), 1.03 (d, J = 6.8 Hz, 3H). |
| 663 | tert-butyl (2R,5S)-2,5-dimethyl- | LC/MS ESI (m/z): 477 (M + H)⁺. ¹H NMR (400 MHz, |
| | 4-(7-(pyridin-2-yl)-5- | CDCl3) δ 8.74 (d, J = 8.3 Hz, 1H), 8.61 (s, 1H), 8.56 (s, |
| | (trifluoromethyl)-7H- | 1H), 8.54-8.50 (m, 1H), 7.97-7.88 (m, 1H), 7.31-7.27 |
| | pyrrolo[2,3-d]pyrimidin-4- | (m, 1H), 4.70-4.25 (m, 2H), 3.82-3.62 (m, 3H), 3.47 (d, |
| | yl)piperazine-1-carboxylate | J = 13.5 Hz, 1H), 1.49 (s, 9H), 1.19-1.09 (m, 6H). |
| 664 | tert-butyl (2R,5S)-4-(7-(4- | LC/MS ESI (m/z): 507 (M + H)⁺. ¹H NMR (400 MHz, |
| | methoxypyridin-2-yl)-5- | CDCl3) δ 8.61 (s, 1H), 8.55 (s, 1H), 8.40 (d, J = 2.0 Hz, |
| | (trifluoromethyl)-7H- | 1H), 8.31 (d, J = 5.7 Hz, 1H), 6.85-6.78 (m, 1H), 4.59- |
| | pyrrolo[2,3-d]pyrimidin-4-yl)- | 4.35 (m, 2H), 3.98 (s, 3H), 3.80-3.63 (m, 3H), 3.49-3.42 |
| | 2,5-dimethylpiperazine-1- | (m, 1H), 1.49 (s, 9H), 1.17-1.09 (m, 6H). |
| | carboxylate | |
| 665 | tert-butyl (2R,5S)-2,5-dimethyl- | LC/MS ESI (m/z): 491 (M + H)⁺. ¹H NMR (400 MHz, |
| | 4-(7-(4-methylpyridin-2-yl)-5- | CDCl3) δ 8.59-8.54 (m, 2H), 8.51 (s, 1H), 8.37 (d, J = 5.0 |
| | (trifluoromethyl)-7H- | Hz, 1H), 7.11 (d, J = 5.0 Hz, 1H), 4.65-4.29 (m, 2H), 3.80- |
| | pyrrolo[2,3-d]pyrimidin-4- | 3.69 (m, 2H), 3.67-3.61 (m, 1H), 3.46 (d, J = 13.2 Hz, |
| | yl)piperazine-1-carboxylate | 1H), 2.51 (s, 3H), 1.49 (s, 9H), 1.16-1.10 (m, 6H). |

### Example 203. Synthesis of tert-butyl (2R,5S)-4-(7-(3-fluorophenyl)-5-methyl-7H-pyrrolo[2,3-d]pyrimidin-4-yl)-2,5-dimethylpiperazine-1-carboxylate (Compound 666)

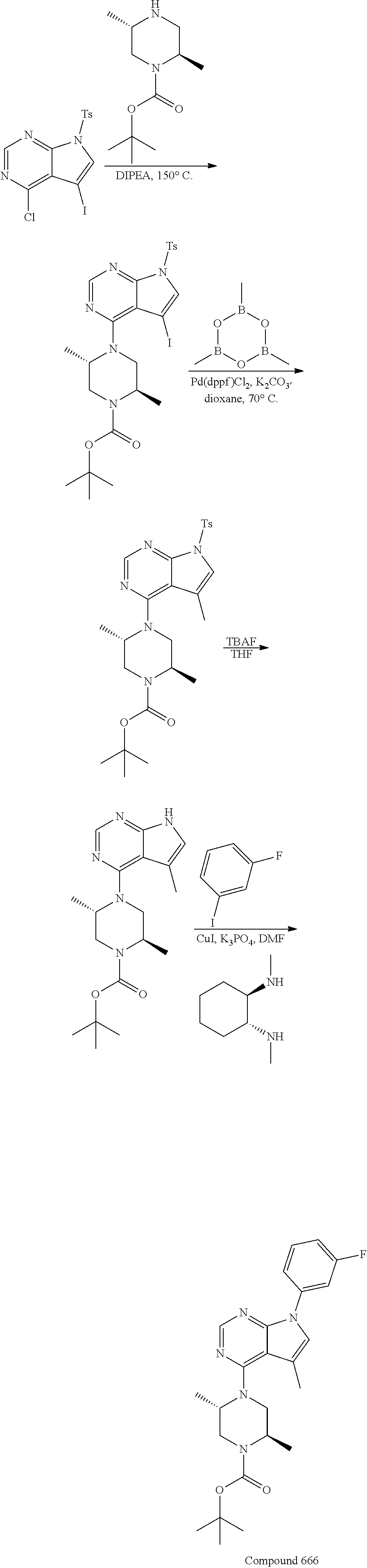

### Step 1. tert-Butyl (2R,5S)-4-(5-iodo-7-tosyl-7H-pyrrolo[2,3-d]pyrimidin-4-yl)-2,5-dimethylpiperazine-1-carboxylate

To a solution of 4-chloro-5-iodo-7-tosyl-7H-pyrrolo[2,3-d]pyrimidine (10 g, 23 mmol, prepared following the procedure outlined for compound 602) in DIPEA (100 mL) was added tert-butyl (2R,5S)-2,5-dimethylpiperazine-1-carboxylate (9.9 g, 46 mmol). The resulting mixture was stirred at 150° C. for 2 h. After cooling to room temperature, the reaction was concentrated and purified by flash column chromatography (silica gel, 0˜30% ethyl acetate in petroleum ether) to afford tert-butyl (2R,5S)-4-(5-iodo-7-tosyl-7H-pyrrolo[2,3-d]pyrimidin-4-yl)-2,5-dimethylpiperazine-1-carboxylate (13 g, 92%) as a yellow solid. LC/MS ESI (m/z): 612 (M+H)⁺.

### Step 2. tert-Butyl (2R,5S)-2,5-dimethyl-4-(5-methyl-7-tosyl-7H-pyrrolo[2,3-d]pyrimidin-4-yl)piperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (4.0 g, 6.7 mmol) in dioxane (50 mL) and H₂O (10 mL) were added 2,4,6-trimethyl-1,3,5,2,4,6-trioxatriborinane (1.7 g, 13 mmol), K₂CO₃ (2.8 g, 20 mmol) and Pd(dppf)Cl₂ (490 mg, 0.67 mmol). The resulting mixture was heated to 70° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0-50% ethyl acetate in petroleum ether) to afford tert-butyl (2R,5S)-2,5-dimethyl-4-(5-methyl-7-tosyl-7H-pyrrolo[2,3-d]pyrimidin-4-yl)piperazine-1-carboxylate (2.0 g, 61%) as a white solid. LC/MS ESI (m/z): 500 (M+H)⁺.

### Step 3. tert-Butyl (2R,5S)-2,5-dimethyl-4-(5-methyl-7H-pyrrolo[2,3-d]pyrimidin-4-yl)piperazine-1-carboxylate

To a solution of tert-butyl (2R,5S)-2,5-dimethyl-4-(5-methyl-7-tosyl-7H-pyrrolo[2,3-d]pyrimidin-4-yl)piperazine-1-carboxylate (2.0 g, 4.0 mmol) in THE (20 mL) was added TBAF (24 mL, 1.0 M in THF). After 5 h, the reaction was quenched with water and extracted twice with EtOAc. The combined organic layers were washed with brine, dried over Na₂SO₄, filtered and concentrated. The residue was purified by flash column chromatography (silica gel, 0˜80% ethyl acetate in petroleum ether) to afford tert-butyl (2R,5S)-2,5-dimethyl-4-(5-methyl-7H-pyrrolo[2,3-d]pyrimidin-4-yl)piperazine-1-carboxylate (1.2 g, 87%) as a white solid. LC/MS ESI (m/z): 346 (M+H)⁺.

### Step 4. tert-Butyl (2R,5S)-4-(7-(3-fluorophenyl)-5-methyl-7H-pyrrolo[2,3-d]pyrimidin-4-yl)-2,5-dimethylpiperazine-1-carboxylate

To a solution of tert-butyl (2R,5S)-2,5-dimethyl-4-(5-methyl-7H-pyrrolo[2,3-d]pyrimidin-4-yl)piperazine-1-carboxylate (50 mg, 0.15 mmol) in DMF (5 mL) were added 1-fluoro-3-iodobenzene (67 mg, 0.30 mmol), trans-N,N′-dimethylcyclohexane-1,2-diamine (11 mg, 0.08 mmol), CuI (14 mg, 0.08 mmol) and K₃PO₄ (95 mg, 0.45 mmol). The resulting mixture was heated to 120° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to give crude product which was further purified by prep-HPLC to afford tert-butyl (2R,5S)-4-(7-(3-fluorophenyl)-5-methyl-7H-pyrrolo[2,3-d]pyrimidin-4-yl)-2,5-dimethylpiperazine-1-carboxylate (45 mg, 710) as a white solid. LC/MS ESI (m/z): 440 (M+H)⁺. ¹H NMR (400 MHz, CD₃OD) δ 8.29 (s, 1H), 7.62 (d, J=10.3 Hz, 1H), 7.52 (m, 2H), 7.39 (s, 1H), 7.11 (m, 1H), 4.54 (s, 1H), 4.38 (s, 1H), 3.77 (9, 2H), 3.65 (d, J=12.4 Hz, 1H), 3.56 (d, J=13.2 Hz, 1H), 2.49 (s, 3H), 1.50 (s, 9H), 1.19-1.13 (m, 6H).

The following compounds were prepared by procedures analogous to the synthesis of compound 666 from the corresponding aryl halides and amines.

| Cmpd | | |
|---|---|---|
| No. | Chemical Name | LCMS and ¹H NMR |
| 667 | tert-butyl (2R,5S)-2,5-dimethyl- | LC/MS ESI (m/z): 490 (M + H)⁺. ¹H NMR (400 MHz, |
| | 4-(5-methyl-7-(3- | CD₃OD) δ 8.31 (s, 1H), 8.14 (s, 1H), 7.99 (d, J = 8.0 Hz, |
| | (trifluoromethyl)phenyl)-7H- | 1H), 7.75-7.64 (m, 2H), 7.45 (s, 1H), 4.55 (s, 1H), 4.39 (s, |
| | pyrrolo[2,3-d]pyrimidin-4- | 1H), 3.79 (d, J = 13.5 Hz, 2H), 3.65 (d, J = 13.5 Hz, 1H), |
| | yl)piperazine-1-carboxylate | 3.57 (d, J = 13.4 Hz, 1H), 2.50 (s, 3H), 1.50 (s, 9H), 1.17 (t, |
| | | J = 7.5 Hz, 6H). |
| 668 | tert-butyl (2R,5S)-2,5-dimethyl- | LC/MS ESI (m/z): 422 (M + H)⁺. ¹H NMR (400 MHz, |
| | 4-(5-methyl-7-phenyl-7H- | CD₃OD) δ 8.25 (s, 1H), 7.65 (d, J = 8.2 Hz, 2H), 7.53 (t, J = |
| | pyrrolo[2,3-d]pyrimidin-4- | 7.7 Hz, 2H), 7.41-7.33 (m, 2H), 4.58-4.51 (m, 1H), |
| | yl)piperazine-1-carboxylate | 4.42-4.34 (m, 1H), 3.79 (d, J = 13.1 Hz, 2H), 3.66 (d, J = |
| | | 11.8 Hz, 1H), 3.56 (d, J = 13.0 Hz, 1H), 2.50 (s, 3H), 1.50 |
| | | (s, 9H), 1.20-1.14 (m, 6H). |
| 669 | tert-butyl (2R,5S)-4-(7-(3- | LC/MS ESI (m/z): 447 (M + H)⁺. ¹H NMR (400 MHz, |
| | cyanophenyl)-5-methyl-7H- | CD₃OD) δ 8.32 (s, 1H), 8.23 (s, 1H), 8.10-8.05 (m, 1H), |
| | pyrrolo[2,3-d]pyrimidin-4-yl)- | 7.73-7.67 (m, 2H), 7.45 (s, 1H), 4.59-4.51 (m, 1H), 4.38 |
| | 2,5-dimethylpiperazine-1- | (s, 1H), 3.82-3.76 (m, 2H), 3.65 (d, J = 12.2 Hz, 1H), 3.57 |
| | carboxylate | (d, J = 13.2 Hz, 1H), 2.50 (s, 3H), 1.50 (s, 9H), 1.16 (t, J = |
| | | 6.3 Hz, 6H). |
| 670 | tert-butyl (2R,5S)-4-(7-(3,5- | LC/MS ESI (m/z): 458 (M + H)⁺. ¹H NMR (400 MHz, |
| | difluorophenyl)-5-methyl-7H- | CD₃OD) δ 8.33 (s, 1H), 7.56 (d, J = 8.4 Hz, 2H), 7.44 (s, |
| | pyrrolo[2,3-d]pyrimidin-4-yl)- | 1H), 6.97-6.90 (m, 1H), 4.53 (s, 1H), 4.38 (s, 1H), 3.78 (d, |
| | 2,5-dimethylpiperazine-1- | J = 13.4 Hz, 2H), 3.64 (d, J = 12.6 Hz, 1H), 3.55 (d, J = |
| | carboxylate | 13.2 Hz, 1H), 2.48 (s, 3H), 1.50 (s, 9H), 1.16 (t, J = 6.9 Hz, |
| | | 6H). |
| 671 | tert-butyl (2R,5S)-4-(7-(3- | LC/MS ESI (m/z): 456 (M + H)⁺. ¹H NMR (400 MHz, |
| | chlorophenyl)-5-methyl-7H- | CD₃OD) δ 8.29 (s, 1H), 7.84 (d, J = 1.5 Hz, 1H), 7.65 (d, J = |
| | pyrrolo[2,3-d]pyrimidin-4-yl)- | 8.1 Hz, 1H), 7.50 (t, J = 7.9 Hz, 1H), 7.37 (m, 2H), 4.55 |
| | 2,5-dimethylpiperazine-1- | (s, 1H), 4.38 (s, 1H), 3.79 (d, J = 13.5 Hz, 2H), 3.65 (d, J = |
| | carboxylate | 12.5 Hz, 1H), 3.56 (d, J = 13.3 Hz, 1H), 2.49 (s, 3H), 1.50 |
| | | (s, 9H), 1.19-1.14 (m, 6H). |
| 672 | tert-butyl (2R,5S)-4-(7-(5- | LC/MS ESI (m/z): 441 (M + H)⁺. ¹H NMR (400 MHz, |
| | fluoropyridin-3-yl)-5-methyl- | CD₃OD) δ 8.93 (s, 1H), 8.45 (s, 1H), 8.34 (s, 1H), 8.30- |
| | 7H-pyrrolo[2,3-d]pyrimidin-4- | 8.25 (m, 1H), 7.51 (s, 1H), 4.56 (s, 1H), 4.38 (s, 1H), 3.83- |
| | yl)-2,5-dimethylpiperazine-1- | 3.76 (m, 2H), 3.68-3.55 (m, 2H), 2.50 (s, 3H), 1.50 (s, |
| | carboxylate | 9H), 1.16 (d, J = 6.7 Hz, 6H). |
| 673 | tert-butyl (2R,5S)-2,5-dimethyl- | LC/MS ESI (m/z): 423 (M + H)⁺. ¹H NMR (400 MHz, |
| | 4-(5-methyl-7-(pyridin-2-yl)- | CD₃OD) δ 8.61 (d, J = 8.3 Hz, 1H), 8.48 (d, J = 4.8 Hz, |
| | 7H-pyrrolo[2,3-d]pyrimidin-4- | 1H), 8.37 (s, 1H), 7.98-7.89 (m, 2H), 7.34-7.23 (m, 1H), |
| | yl)piperazine-1-carboxylate | 4.51 (s, 1H), 4.39 (s, 1H), 3.78 (d, J = 13.2 Hz, 2H), 3.66 |
| | | (d, J = 12.2 Hz, 1H), 3.52 (d, J = 13.3 Hz, 1H), 2.49 (s, |
| | | 3H), 1.50 (s, 9H), 1.18 (d, J = 6.7 Hz, 3H), 1.14 (d, J = 6.7 |
| | | Hz, 3H). |
| 674 | tert-butyl (2R,5S)-2,5-dimethyl- | LC/MS ESI (m/z): 424 (M + H)⁺. ¹H NMR (400 MHz, |
| | 4-(5-methyl-7-(pyrimidin-5-yl)- | CD₃OD) δ 9.33 (s, 2H), 9.10 (s, 1H), 8.35 (s, 1H), 7.54 (s, |
| | 7H-pyrrolo[2,3-d]pyrimidin-4- | 1H), 4.57 (s, 1H), 4.38 (s, 1H), 3.94-3.76 (m, 2H), 3.62 |
| | yl)piperazine-1-carboxylate | (m, 2H), 2.51 (s, 3H), 1.50 (s, 9H), 1.18-1.14 (m, 6H). |
| 675 | tert-butyl (2R,5S)-4-(7-(5- | LC/MS ESI (m/z): 448 (M + H)⁺. ¹H NMR (400 MHz, |
| | cyanopyridin-3-yl)-5-methyl- | CD₃OD) δ 9.34 (d, J = 2.4 Hz, 1H), 8.84 (d, J = 1.3 Hz, |
| | 7H-pyrrolo[2,3-d]pyrimidin-4- | 1H), 8.76 (d, J = 1.8 Hz, 1H), 8.36 (s, 1H), 7.55 (s, 1H), |
| | yl)-2,5-dimethylpiperazine-1- | 4.60-4.54 (m, 1H), 4.38 (s, 1H), 3.83-3.76 (m, 2H), 3.61 |
| | carboxylate | (m, 2H), 2.50 (s, 3H), 1.50 (s, 9H), 1.18-1.14 (m, 6H). |
| 676 | tert-butyl (2R,5S)-4-(7-(4- | LC/MS ESI (m/z): 440 (M + H)⁺. ¹H NMR (400 MHz, |
| | fluorophenyl)-5-methyl-7H- | CD₃OD) δ 8.25 (s, 1H), 7.69-7.64 (m, 2H), 7.32-7.24 |
| | pyrrolo[2,3-d]pyrimidin-4-yl)- | (m, 3H), 4.58-4.52 (m, 1H), 4.42-4.34 (m, 1H), 3.79 (d, |
| | 2,5-dimethylpiperazine-1- | J = 13.2 Hz, 2H), 3.68-3.54 (m, 2H), 2.49 (s, 3H), 1.50 (s, |
| | carboxylate | 9H), 1.19-1.13 (m, 6H). |
| 677 | tert-butyl (2R,5S)-4-(7-(3- | LC/MS ESI (m/z): 474 (M + H)⁺. ¹H NMR (400 MHz, |
| | chloro-5-fluorophenyl)-5- | CD₃OD) δ 8.33 (s, 1H), 7.77 (s, 1H), 7.66 (d, J = 10.1Hz, |
| | methyl-7H-pyrrolo[2,3- | 1H), 7.44 (s, 1H), 7.19 (d, J = 8.4 Hz, 1H), 4.57-4.50 (m, |
| | d]pyrimidin-4-yl)-2,5- | 1H), 4.38 (s, 1H), 3.81-3.75 (m, 2H), 3.64 (d, J = 12.2 Hz, |
| | dimethylpiperazine-1- | 1H), 3.55 (d, J = 13.3 Hz, 1H), 2.48 (s, 3H), 1.50 (s, 9H), |
| | carboxylate | 1.15 (m, 6H). |
| 678 | tert-butyl (R)-4-(7-(4- | LC/MS ESI (m/z): 427 (M + H)⁺. ¹H NMR (400 MHz, |
| | fluoropyridin-2-yl)-5-methyl- | CD₃OD) δ 8.65 (d, J = 10.8 Hz, 1H), 8.42 (m, 2H), 7.98 (s, |
| | 7H-pyrrolo[2,3-d]pyrimidin-4- | 1H), 7.07 (s, 1H), 4.43-4.36 (m, 1H), 4.04 (d, J = 12.8 Hz, |
| | yl)-2-methylpiperazine-1- | 1H), 3.94 (d, J = 13.2 Hz, 1H), 3.83 (d, J = 12.9 Hz, 1H), |
| | carboxylate | 3.48-3.41 (m, 1H), 3.37-3.32 (m, 1H), 3.09-3.01 (m, |
| | | 1H), 2.47 (s, 3H), 1.49 (s, 9H), 1.22 (d, J = 6.8 Hz, 3H). |
| 679 | tert-butyl (R)-2-methyl-4-(5- | LC/MS ESI (m/z): 477 (M + H)⁺. ¹H NMR (400 MHz, |
| | methyl-7-(4- | CD₃OD) δ 9.21 (s, 1H), 8.67 (d, J = 5.1 Hz, 1H), 8.43 (s, |
| | (trifluoromethyl)pyridin-2-yl)- | 1H), 8.04 (s, 1H), 7.50 (d, J = 5.1 Hz, 1H), 4.46-4.32 (m, |
| | 7H-pyrrolo[2,3-d]pyrimidin-4- | 1H), 4.06 (d, J = 12.9 Hz, 1H), 3.95 (d, J = 13.4 Hz, 1H), |
| | yl)piperazine-1-carboxylatee | 3.85 (d, J = 12.9 Hz, 1H), 3.52-3.38 (m, 1H), 3.38-3.32 |
| | | (m, 1H), 3.11-2.98 (m, 1H), 2.48 (s, 3H), 1.49 (s, 9H), |
| | | 1.23 (d, J = 6.7 Hz, 3H). |
| 680 | tert-butyl (R)-4-(7-(4- | LC/MS ESI (m/z): 434 (M + H)⁺. ¹H NMR (400 MHz, |
| | cyanopyridin-2-yl)-5-methyl- | CDCl₃) δ 9.32 (s, 1H), 8.59 (d, J = 5.0 Hz, 1H), 8.51 (s, |
| | 7H-pyrrolo[2,3-d]pyrimidin-4- | 1H), 7.97 (s, 1H), 7.35 (d, J = 5.0 Hz, 1H), 4.41 (s, 1H), |
| | yl)-2-methylpiperazine-1- | 4.04-3.95 (m, 2H), 3.84 (d, J = 12.7 Hz, 1H), 3.47-3.39 |
| | carboxylate | (m, 1H), 3.37-3.32 (m, 1H), 3.12-3.05 (m, 1H), 2.47 (s, |
| | | 3H), 1.50 (s, 9H), 1.23 (d, J = 6.8 Hz, 3H). |
| 681 | tert-butyl (S)-4-(7-(4- | LC/MS ESI (m/z): 427 (M + H)⁺. ¹H NMR (400 MHz, |
| | fluoropyridin-2-yl)-5-methyl- | CDCl₃) δ 8.71 (dd, J = 11.2, 2.4 Hz, 1H), 8.50 (s, 1H), 8.40 |
| | 7H-pyrrolo[2,3-d]pyrimidin-4- | (dd, J = 8.6, 5.6 Hz, 1H), 7.97 (d, J = 1.2 Hz, 1H), 6.92- |
| | yl)-3-methylpiperazine-1- | 6.85 (m, 1H), 4.29 (m, 1H), 4.07-3.76 (m, 2H), 3.62 (m, |
| | carboxylate | 1H), 3.50 (m, 1H), 3.30 (m, 2H), 2.44 (d, J = 0.8 Hz, 3H), |
| | | 1.50 (s, 9H), 1.22 (d, J = 6.6 Hz, 3H). |
| 682 | tert-butyl (S)-3-methyl-4-(5- | LC/MS ESI (m/z): 477 (M + H)⁺. ¹H NMR (400 MHz, |
| | methyl-7-(4- | CDCl₃) δ 9.24 (s, 1H), 8.60 (d, J = 5.2 Hz, 1H), 8.52 (s, |
| | (trifluoromethyl)pyridin-2-yl)- | 1H), 7.99 (d, J = 1.0 Hz, 1H), 7.36 (dd, J = 5.2, 0.8 Hz, |
| | 7H-pyrrolo[2,3-d]pyrimidin-4- | 1H), 4.29 (m, 1H), 4.08-3.75 (m, 2H), 3.63 (d, J = 12.6 |
| | yl)piperazine-1-carboxylate | Hz, 1H), 3.50 (m, 1H), 3.30 (m, 2H), 2.45 (d, J = 0.8 Hz, |
| | | 3H), 1.50 (s, 9H), 1.23 (d, J = 6.6 Hz, 3H) |
| 683 | tert-butyl (S)-4-(7-(3- | LC/MS ESI (m/z): 426 (M + H)⁺. ¹H NMR (400 MHz, |
| | fluorophenyl)-5-methyl-7H- | CDCl₃) δ 8.46 (s, 1H), 7.52-7.48 (m, 2H), 7.47-7.42 (m, |
| | pyrrolo[2,3-d]pyrimidin-4-yl)-3- | 1H), 7.11 (s, 1H), 7.06-7.00 (m, 1H), 4.31 (m, 1H), 3.93 |
| | methylpiperazine-1-carboxylate | (m, 2H), 3.65 (d, J = 13.0 Hz, 1H), 3.51 (m, 1H), 3.30 (m, |
| | | 2H), 2.46 (s, 3H), 1.50 (s, 9H), 1.24 (d, J = 6.6 Hz, 3H) |
| 684 | tert-butyl (S)-3-methyl-4-(5- | LC/MS ESI (m/z): 476 (M + H)⁺. ¹H NMR (400 MHz, |
| | methyl-7-(3- | CDCl₃) δ 8.46 (s, 1H), 7.98 (d, J = 8.0 Hz, 1H), 7.92 (s, |
| | (trifluoromethyl)phenyl)-7H- | 1H), 7.64 (t, J = 7.8 Hz, 1H), 7.58 (d, J = 7.8 Hz, 1H), 7.15 |
| | pyrrolo[2,3-d]pyrimidin-4- | (s, 1H), 4.38-4.28 (m, 1H), 3.94 (m, 2H), 3.67 (d, J = 12.6 |
| | yl)piperazine-1-carboxylate | Hz, 1H), 3.51 (m, 1H), 3.29 (m, 2H), 2.47 (s, 3H), 1.50 (s, |
| | | 9H), 1.24 (d, J = 6.6 Hz, 3H). |
| 685 | tert-butyl | LC/MS ESI (m/z): 408 (M + H)⁺. ¹H NMR (400 MHz, |
| | (S)-3-methyl-4-(5-methyl-7- | CDCl₃) δ 8.46 (s, 1H), 7.65 (dd, J = 8.5, 1.0 Hz, 2H), 7.50 |
| | phenyl-7H-pyrrolo[2,3- | (t, J = 7.8 Hz, 2H), 7.34 (t, J = 7.4 Hz, 1H), 7.12 (d, J = 0.8 |
| | d]pyrimidin-4-yl)piperazine-1- | Hz, 1H), 4.32 (m, 1H), 3.92 (m, 2H), 3.65 (d, J = 13.2 Hz, |
| | carboxylate | 1H), 3.51 (m, 1H), 3.31 (m, 2H), 2.47 (s, 3H), 1.50 (s, 9H), |
| | | 1.23 (d, J = 6.6 Hz, 3H). |
| 686 | tert-butyl | LC/MS ESI (m/z): 433 (M + H)⁺. ¹H NMR (400 MHz, |
| | (S)-4-(7-(3-cyanophenyl)-5- | CDCl₃) δ 8.45 (s, 1H), 8.07 (d, J = 1.0 Hz, 1H), 8.01 (m, |
| | methyl-7H-pyrrolo[2,3- | 1H), 7.63-7.58 (m, 2H), 7.12 (d, J = 0.8 Hz, 1H), 4.34 (m, |
| | d]pyrimidin-4-yl)-3- | 1H), 3.95 (m, 2H), 3.68 (d, J = 13.0 Hz, 1H), 3.52 (m, 1H), |
| | methylpiperazine-1-carboxylate | 3.28 (m, 2H), 2.46 (s, 3H), 1.50 (s, 9H), 1.25 (d, J = 6.6 Hz, |
| | | 3H). |
| 687 | tert-butyl | LC/MS ESI (m/z): 444 (M + H)⁺. ¹H NMR(400 MHz, |
| | (S)-4-(7-(3,5-difluorophenyl)-5- | CDCl₃) δ 8.46 (s, 1H), 7.38 (dd, J = 8.4, 2.2 Hz, 2H), 7.10 |
| | methyl-7H-pyrrolo[2,3- | (d, J = 1.0 Hz, 1H), 6.77 (m, 1H), 4.37-4.28 (m, 1H), 3.94 |
| | d]pyrimidin-4-yl)-3- | (m, 2H), 3.65 (d, J = 13.0 Hz, 1H), 3.51 (m, 1H), 3.29 (m, |
| | methylpiperazine-1-carboxylate | 2H), 2.45 (s, 3H), 1.50 (s, 9H), 1.24 (d, J = 6.6 Hz, 3H). |
| 688 | tert-butyl | LC/MS ESI (m/z): 442 (M + H)⁺. ¹H NMR (400 MHz, |
| | (S)-4-(7-(3-chlorophenyl)-5- | CDCl₃) δ 8.46 (s, 1H), 7.71 (t, J = 2.0 Hz, 1H), 7.65-7.60 |
| | methyl-7H-pyrrolo[2,3- | (m, 1H), 7.43 (t, J = 8.2Hz, 1H), 7.32-7.28 (m, 1H), 7.10 |
| | d]pyrimidin-4-yl)-3- | (s, 1H), 4.32 (m, 1H), 4.11-3.76 (m, 2H), 3.65 (d, J =12.8 |
| | methylpiperazine-1-carboxylate | Hz, 1H), 3.51 (m, 1H), 3.29 (m, 2H), 2.45 (s, 3H), 1.50 (s, |
| | | 9H), 1.24 (d, J = 6.6 Hz, 3H). |
| 689 | tert-butyl | LC/MS ESI (m/z): 427 (M + H)⁺. ¹H NMR (400 MHz, |
| | (S)-4-(7-(5-fluoropyridin-3-yl)- | CDCl₃) δ 8.75 (s, 1H), 8.46 (s, 1H), 8.44 (d, J = 2.5 Hz, |
| | 5-methyl-7H-pyrrolo[2,3- | 1H), 8.14 (dt, J = 9.8, 2.4 Hz, 1H), 7.16 (d, J = 1.0 Hz, 1H), |
| | d]pyrimidin-4-yl)-3- | 4.38-4.31 (m, 1H), 3.95 (m, 2H), 3.68 (d, J = 13.6 Hz, |
| | methylpiperazine-1-carboxylate | 1H), 3.51 (m, 1H), 3.28 (m, 2H), 2.47 (s, 3H), 1.50 (s, 9H), |
| | | 1.25 (d, J = 6.6 Hz, 3H). |
| 690 | tert-butyl | LC/MS ESI (m/z): 409 (M + H)⁺. ¹H NMR (400 MHz, |
| | (S)-3-methyl-4-(5-methyl-7- | CDCl₃) δ 8.71 (d, J=8.3 Hz, 1H), 8.50 (s, 1H), 8.49-8.45 |
| | (pyridin-2-yl)-7H-pyrrolo[2,3- | (m, 1H), 7.94 (d, J = 1.1 Hz, 1H), 7.86 (m, 1H), 7.17 (m, |
| | d]pyrimidin-4-yl)piperazine-1- | 1H), 4.38-4.29 (m, 1H), 4.20-3.70 (m, 2H), 3.60 (m, |
| | carboxylate | 2H), 3.50 (m, 1H), 3.31 (m, 1H), 2.46 (d, J = 0.9 Hz, 3H), |
| | | 1.50 (s, 9H), 1.22 (d, J = 6.6 Hz, 3H). |
| 691 | tert-butyl | LC/MS ESI (m/z): 410 (M + H)⁺. ¹H NMR (400 MHz, |
| | (S)-3-methyl-4-(5-methyl-7- | CDCl₃) δ 9.22 (s, 2H), 9.16 (s, 1H), 8.45 (s, 1H), 7.13 (d, J = |
| | (pyrimidin-5-yl)-7H-pyrrolo[2,3- | 1.0 Hz, 1H), 4.40-4.33 (m, 1H), 3.96 (m, 2H), 3.70 (d, J = |
| | d]pyrimidin-4-yl)piperazine-1- | 13.0 Hz, 1H), 3.52 (m, 1H), 3.27 (m, 2H), 2.47 (d, J = 0.8 |
| | carboxylate | Hz, 3H), 1.50 (s, 9H), 1.26 (d, J = 6.6 Hz, 3H). |
| 692 | tert-butyl | LC/MS ESI (m/z): 426 (M + H)⁺. ¹H NMR (400 MHz, |
| | (S)-4-(7-(4-fluorophenyl)-5- | CDCl₃) δ 8.44 (s, 1H), 7.62-7.58 (m, 2H), 7.19 (t, J = 8.6 |
| | methyl-7H-pyrrolo[2,3- | Hz, 2H), 7.06 (d, J = 0.8 Hz, 1H), 4.33 (m, 1H), 4.20-3.70 |
| | d]pyrimidin-4-yl)-3- | (m, 2H), 3.66 (d, J = 13.0 Hz, 1H), 3.51 (m, 1H), 3.30 (m, |
| | methylpiperazine-1-carboxylate | 2H), 2.46 (s, 3H), 1.50 (s, 9H), 1.24 (d, J = 6.6 Hz, 3H). |
| 693 | tert-butyl | LC/MS ESI (m/z): 460 (M + H)⁺. ¹H NMR (400 MHz, |
| | (S)-4-(7-(3-chloro-5- | CDCl₃) δ 8.46 (s, 1H), 7.59-7.55 (m, 1H), 7.55-7.49 (m, |
| | fluorophenyl)-5-methyl-7H- | 1H), 7.09 (d, J = 1.0 Hz, 1H), 7.04 (dt, J = 8.2, 2.2 Hz, 1H), |
| | pyrrolo[2,3-d]pyrimidin-4-yl)-3- | 4.32 (m, 1H), 3.94 (m, 2H), 3.65 (d, J = 13.0 Hz, 1H), 3.50 |
| | methylpiperazine-1-carboxylate | (m, 1H), 3.28 (m, 2H), 2.44 (d, J = 0.8 Hz, 3H), 1.50 (s, |
| | | 9H), 1.24 (d, J = 6.6 Hz, 3H). |
| 694 | tert-butyl | LC/MS ESI (m/z): 434 (M + H)⁺. ¹H NMR (400 MHz, |
| | (S)-4-(7-(5-cyanopyridin-3-yl)- | CDCl₃) δ 9.16 (d, J = 2.4 Hz, 1H), 8.79 (d, J = 1.8 Hz, 1H), |
| | 5-methyl-7H-pyrrolo[2,3- | 8.68-8.65 (m, 1H), 8.45 (s, 1H), 7.16 (d, J = 1.0 Hz, 1H), |
| | d]pyrimidin-4-yl)-3- | 4.41-4.33 (m, 1H), 3.97 (m, 2H), 3.71 (d, J = 13.6 Hz, |
| | methylpiperazine-1-carboxylate | 1H), 3.52 (m, 1H), 3.26 (m, 2H), 2.47 (s, 3H), 1.50 (s, 9H), |
| | | 1.26 (d, J = 6.6 Hz, 3H). |

### Example 204. Synthesis of tert-butyl (2R,5S)-4-(5-(difluoromethyl)-7-(3-fluorophenyl)-7H-pyrrolo[2,3-d]pyrimidin-4-yl)-2,5-dimethylpiperazine-1-carboxylate (Compound 695)

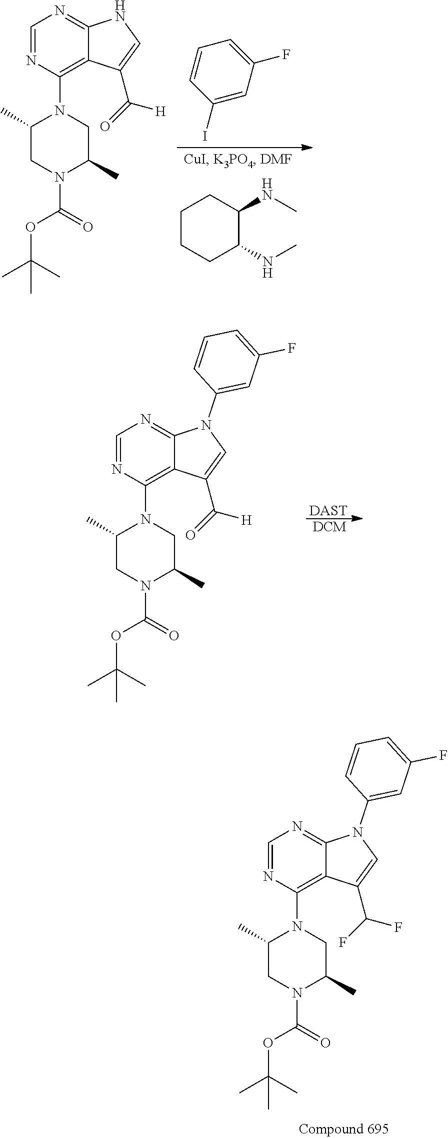

### Step 1. tert-Butyl (2R,5S)-4-(7-(3-fluorophenyl)-5-formyl-7H-pyrrolo[2,3-d]pyrimidin-4-yl)-2,5-dimethylpiperazine-1-carboxylate

To a solution of tert-butyl (2R,5S)-4-(5-formyl-7H-pyrrolo[2,3-d]pyrimidin-4-yl)-2,5-dimethylpiperazine-1-carboxylate (400 mg, 1.1 mmol, prepared following the procedure outlined for compound 633) in DMF (5 mL) were added 1-fluoro-3-iodobenzene (490 mg, 2.2 mmol), trans-N,N′-dimethylcyclohexane-1,2-diamine (78 mg, 0.55 mmol), CuI (105 mg, 0.55 mmol) and K₃PO₄ (1.4 g, 6.7 mmol). The resulting mixture was heated to 120° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-4-(7-(3-fluorophenyl)-5-formyl-7H-pyrrolo[2,3-d]pyrimidin-4-yl)-2,5-dimethylpiperazine-1-carboxylate (280 mg, 56%) as a white solid. LC/MS ESI (m/z): 454 (M+H)⁺

### Step 2. tert-Butyl (2R,5S)-4-(5-(difluoromethyl)-7-(3-fluorophenyl)-7H-pyrrolo[2,3-d]pyrimidin-4-yl)-2,5-dimethylpiperazine-1-carboxylate

To a solution of tert-butyl (2R,5S)-4-(7-(3-fluorophenyl)-5-formyl-7H-pyrrolo[2,3-d]pyrimidin-4-yl)-2,5-dimethylpiperazine-1-carboxylate (280 mg, 0.61 mmol) in DCM (10 mL) at 0° C. was added BAST (680 mg, 3.1 mmol) dropwise. The resulting mixture was stirred at 40° C. overnight. After cooling to room temperature, the reaction was partitioned between DCM and NaHCO₃ (aq.). The aqueous layer was extracted twice with DCM. The combined organic layers were washed with brine, dried over Na₂SO₄, filtered and concentrated. the residue was purified by prep-HPLC to afford tert-butyl (2R,5S)-4-(5-(difluoromethyl)-7-(3-fluorophenyl)-7H-pyrrolo[2,3-d]pyrimidin-4-yl)-2,5-dimethylpiperazine-1-carboxylate (140 mg, 48%) as a white solid. LC/MS ESI (m/z): 476 (M+H)⁺. ¹H NMR (400 MHz, CD₃OD) δ 8.39 (s, 1H), 7.95 (d, J=1.6 Hz, 1H), 7.65-7.61 (m, 1H), 7.59-7.53 (m, 2H), 7.31-7.01 (m, 2H), 4.69-4.62 (m, 1H), 4.37 (s, 1H), 3.84-3.73 (m, 2H), 3.62 (m, 2H), 1.50 (s, 9H), 1.18-1.12 (m, 6H).

### Example 205. Synthesis of tert-butyl (2R,5S)-4-(5-ethyl-7-(4-(trifluoromethyl)pyridin-2-yl)-7H-pyrrolo[2,3-d]pyrimidin-4-yl)-2,5-dimethylpiperazine-1-carboxylate (Compound 696)

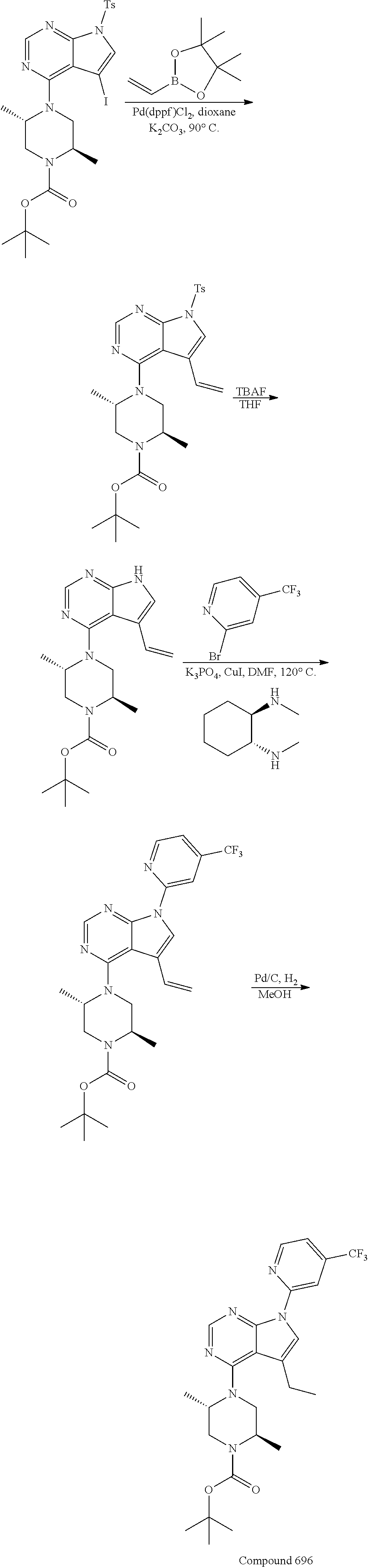

### Step 1. tert-Butyl (2R,5S)-2,5-dimethyl-4-(7-tosyl-5-vinyl-7H-pyrrolo[2,3-d]pyrimidin-4-yl)piperazine-1-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (1.5 g, 2.5 mmol, prepared following the procedure outlined for compound 666) in dioxane (15 mL) and H₂O (3 mL) were added 4,4,5,5-tetramethyl-2-vinyl-1,3,2-dioxaborolane (770 mg, 5.0 mmol), K₂CO₃ (1.0 g, 7.5 mmol) and Pd(dppf)Cl₂ (220 mg, 0.30 mmol). The resulting mixture was heated to 90° C. overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-2,5-dimethyl-4-(7-tosyl-5-vinyl-7H-pyrrolo[2,3-d]pyrimidin-4-yl)piperazine-1-carboxylate (900 mg, 72%) as a white solid. LC/MS ESI (m/z): 512 (M+H)⁺.

### Step 2. tert-Butyl (2R,5S)-2,5-dimethyl-4-(5-vinyl-7H-pyrrolo[2,3-d]pyrimidin-4-yl)piperazine-1-carboxylate

To a solution of tert-butyl (2R,5S)-2,5-dimethyl-4-(7-tosyl-5-vinyl-7H-pyrrolo[2,3-d]pyrimidin-4-yl)piperazine-1-carboxylate (900 mg, 1.8 mmol) in THE (20 mL) was added TBAF (11 mL, 1.0 M in THF). After 5 h, the reaction was diluted with water and extracted twice with EtOAc. The combined organic layers were washed with brine, dried over Na₂SO₄, filtered and concentrated. The residue was purified by flash column chromatography (silica gel, 0˜80% ethyl acetate in petroleum ether) to afford tert-butyl (2R,5S)-2,5-dimethyl-4-(5-vinyl-7H-pyrrolo[2,3-d]pyrimidin-4-yl)piperazine-1-carboxylate (540 mg, 86%) as a white solid. LC/MS ESI (m/z): 358 (M+H)⁺.

### Step 3. tert-Butyl (2R,5S)-2,5-dimethyl-4-(7-(4-(trifluoromethyl)pyridin-2-yl)-5-vinyl-7H-pyrrolo[2,3-d]pyrimidin-4-yl)piperazine-1-carboxylate

To a solution of tert-butyl (2R,5S)-2,5-dimethyl-4-(5-vinyl-7H-pyrrolo[2,3-d]pyrimidin-4-yl)piperazine-1-carboxylate (270 mg, 0.75 mmol) in DMF (5 mL) were added 2-bromo-4-(trifluoromethyl)pyridine (340 mg, 1.5 mmol), trans-N,N-dimethylcyclohexane-1,2-diamine (53 mg, 0.38 mmol), CuI (71 mg, 0.38 mmol) and K₃PO₄ (490 mg, 2.3 mmol). The resulting mixture was heated to 120° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (2R,5S)-2,5-dimethyl-4-(7-(4-(trifluoromethyl)pyridin-2-yl)-5-vinyl-7H-pyrrolo[2,3-d]pyrimidin-4-yl)piperazine-1-carboxylate (160 mg, 42%) as a white solid. LC/MS ESI (m/z): 503 (M+H)⁺.

### Step 4. tert-Butyl (2R,5S)-2,5-dimethyl-4-(7-(4-(trifluoromethyl)pyridin-2-yl)-5-vinyl-7H-pyrrolo[2,3-d]pyrimidin-4-yl)piperazine-1-carboxylate

To a solution of tert-butyl (2R,5S)-2,5-dimethyl-4-(7-(4-(trifluoromethyl)pyridin-2-yl)-5-vinyl-7H-pyrrolo[2,3-d]pyrimidin-4-yl)piperazine-1-carboxylate (160 mg, 0.32 mmol) in MeOH (10 mL) was added Pd/C (100 mg, 10% on carbon, wetted with ca. 5500 water), the resulting mixture was stirred under H₂ (˜0.1 MPa) overnight. The reaction mixture was then filtered and concentrated. The residue was purified by flash column chromatography (silica gel, 0-50% ethyl acetate in petroleum ether) to afford tert-butyl (2R,5S)-4-(5-ethyl-7-(4-(trifluoromethyl)pyridin-2-yl)-7H-pyrrolo[2,3-d]pyrimidin-4-yl)-2,5-dimethylpiperazine-1-carboxylate (42 mg, 260) as a white solid. LC/MS ESI (m/z): 505 (M+H). ¹H NMR (400 MHz, CD₃OD) δ 9.24 (s, 1H), 8.70 (d, J=5.1 Hz, 1H), 8.47 (s, 1H), 8.10 (s, 1H), 7.54-7.51 (m, 1H), 4.54-4.47 (m, 1H), 4.37 (s, 1H), 3.85-3.76 (m, 2H), 3.70-3.63 (m, 1H), 3.51-3.47 (m, 1H), 2.91 (q, J=7.4 Hz, 2H), 1.50 (s, 9H), 1.38 (t, J=7.4 Hz, 3H), 1.14 (m, 6H).

The following compounds were prepared by procedures analogous to the synthesis of compound 696 from the corresponding aryl halides and boronic pinacol esters.

| Cmpd | | |
|---|---|---|
| No. | Chemical Name | LCMS and ¹H NMR |
| 697 | tert-butyl (2R,5S)-4-(5-ethyl-7- | LC/MS ESI (m/z): 455 (M + H)⁺. ¹H NMR (400 MHz, |
| | (4-fluoropyridin-2-yl)-7H- | CD₃OD) δ 8.70-8.64 (m, 1H), 8.49-8.39 (m, 2H), 8.02 |
| | pyrrolo[2,3-d]pyrimidin-4-yl)- | (s, 1H), 7.10-7.04 (m, 1H), 4.52-4.45 (m, 1H), 4.41-4.33 |
| | 2,5-dimethylpiperazine-1- | (m, 1H), 3.83-3.74 (m, 2H), 3.70-3.61 (m, 1H), |
| | carboxylate | 3.49-3.44 (m, 1H), 2.89 (q, J = 7.7 Hz, 2H), 1.50 (s, 9H), |
| | | 1.36 (t, J = 7.4 Hz, 3H), 1.17-1.11 (m, 6H). |
| 698 | tert-butyl (2R,5S)-4-(7-(4- | LC/MS ESI (m/z): 469 (M + H)⁺. ¹H NMR (400 MHz, |
| | fluoropyridin-2-yl)-5-isopropyl- | CD₃OD) δ 8.70 (dd, J = 11.3, 2.3 Hz, 1H), 8.52-8.41 (m, |
| | 7H-pyrrolo[2,3-d]pyrimidin-4- | 2H), 8.05 (s, 1H), 7.14-7.04 (m, 1H), 4.50-4.32 (m, 2H), |
| | yl)-2,5-dimethylpiperazine-1- | 3.86-3.74 (m, 2H), 3.72-3.61 (m, 1H), 3.45 (m, 1H), |
| | carboxylate | 3.30-3.20 (m, 1H), 1.53-1.43 (m, 12H), 1.29 (d, J = 6.7 |
| | | Hz, 3H), 1.15 (d, J = 6.8 Hz, 3H), 1.10 (d, J = 6.6 Hz, 3H). |
| 669 | tert-butyl (2R,5S)-4-(5- | LC/MS ESI (m/z): 519 (M + H)⁺. ¹H NMR (400 MHz, |
| | isopropyl-7-(4- | CD₃OD) δ 9.26 (s, 1H), 8.71 (d, J = 5.1 Hz, 1H), 8.48 (s, |
| | (trifluoromethyl)pyridin-2-yl)- | 1H), 8.10 (s, 1H), 7.53 (d, J = 5.1 Hz, 1H), 4.50-4.32 (m, |
| | 7H-pyrrolo[2,3-d]pyrimidin-4- | 2H), 3.86-3.75 (m, 2H), 3.72-3.62 (m, 1H), 3.45 (m, |
| | yl)-2,5-dimethylpiperazine-1- | 1H), 3.30-3.22 (m, 1H), 1.53-1.47 (m, 12H), 1.30 (d, J = |
| | carboxylate | 6.7 Hz, 3H), 1.17-1.10 (m, 6H). |

### Example 206. Synthesis of tert-butyl (2R,5S)-4-(7-(4-cyanopyridin-2-yl)-5-isopropyl-7H-pyrrolo[2,3-d]pyrimidin-4-yl)-2,5-dimethylpiperazine-1-carboxylate (Compound 700)

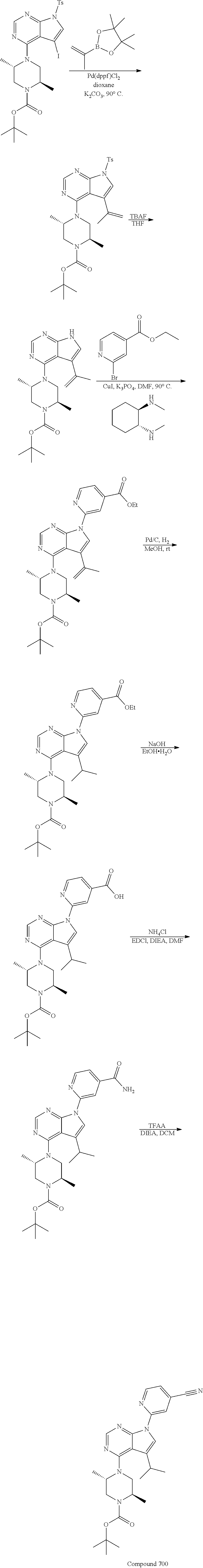

### Step 1. tert-Butyl (2R,5S)-2,5-dimethyl-4-(5-(prop-1-en-2-yl)-7-tosyl-7H-pyrrolo[2,3-d]pyrimidin-4-yl)piperazine-1-carboxylate

The mixture of tert-butyl (2R,5S)-4-(5-iodo-7-tosyl-7H-pyrrolo[2,3-d]pyrimidin-4-yl)-2,5-dimethylpiperazine-1-carboxylate (1.2 g, 2.0 mmol, prepared following the procedure outlined for compound 666), 4,4,5,5-tetramethyl-2-(prop-1-en-2-yl)-1,3,2-dioxaborolane (500 mg, 2.9 mmol), K₂CO₃ (810 mg, 5.9 mmol) and Pd(dppf)Cl₂ (160 mg, 0.20 mmol) in dioxane (20 mL) and water (1 mL) was heated to 90° C. overnight. After cooling to room temperature, the solvent was removed and the residue was purified by flash column chromatography (silica gel, 0˜30% ethyl acetate in petroleum ether) to afford tert-butyl (2R,5S)-2,5-dimethyl-4-(5-(prop-1-en-2-yl)-7-tosyl-7H-pyrrolo[2,3-d]pyrimidin-4-yl)piperazine-1-carboxylate (620 mg, 60%) as a white solid. LC/MS ESI (m/z): 526 (M+H)⁺.

### Step 2. tert-Butyl (2R,5S)-2,5-dimethyl-4-(5-(prop-1-en-2-yl)-7H-pyrrolo[2,3-d]pyrimidin-4-yl)piperazine-1-carboxylate

To a solution of tert-butyl (2R,5S)-2,5-dimethyl-4-(5-(prop-1-en-2-yl)-7-tosyl-7H-pyrrolo[2,3-d]pyrimidin-4-yl)piperazine-1-carboxylate (470 mg, 0.89 mmol) in THE (2 mL) at 0° C. was added TBAF (5.0 mL, 1.0M in THF). After 4 h, the reaction was partitioned between EtOAc and sat. NH₄Cl (aq.). The aqueous layer was extracted with EtOAc. The combined organic layers were washed twice with sat. NH₄Cl (aq.), dried over Na₂SO₄, filtered and concentrated. The residue was purified by flash column chromatography (silica gel, 0˜60% ethyl acetate in petroleum ether) to afford tert-butyl (2R,5S)-2,5-dimethyl-4-(5-(prop-1-en-2-yl)-7H-pyrrolo[2,3-d]pyrimidin-4-yl)piperazine-1-carboxylate (170 mg, 51%). LC/MS ESI (m/z): 372 (M+H)⁺.

### Step 3. tert-Butyl (2R,5S)-4-(7-(4-(ethoxycarbonyl)pyridin-2-yl)-5-(prop-1-en-2-yl)-7H-pyrrolo[2,3-d]pyrimidin-4-yl)-2,5-dimethylpiperazine-1-carboxylate

To a solution of tert-butyl (2R,5S)-2,5-dimethyl-4-(5-(prop-1-en-2-yl)-7H-pyrrolo[2,3-d]pyrimidin-4-yl)piperazine-1-carboxylate (170 mg, 0.46 mmol) in DMF (5 mL) were added ethyl 2-bromoisonicotinate (160 mg, 0.69 mmol), CuI (44 mg, 0.23 mmol), K₃PO₄ (390 mg, 1.8 mmol) and trans-N¹,N²-dimethylcyclohexane-1,2-diamine (33 mg, 0.23 mmol). The resulting mixture was heated to 90° C. for 4 h. After cooling to room temperature, the reaction mixture was partitioned between EtOAc and water. The aqueous layer was extracted twice with EtOAc. The combined organic layers were washed with brine and dried over Na₂SO₄, filtered and concentrated. The residue was purified by flash column chromatography (silica gel, 0˜40% ethyl acetate in petroleum ether) to afford tert-butyl (2R,5S)-4-(7-(4-(ethoxycarbonyl)pyridin-2-yl)-5-(prop-1-en-2-yl)-7H-pyrrolo[2,3-d]pyrimidin-4-yl)-2,5-dimethylpiperazine-1-carboxylate (180 mg, 75%) as a solid. LC/MS ESI (m/z): 521 (M+H)⁺.

### Step 4. tert-Butyl (2R,5S)-4-(7-(4-(ethoxycarbonyl)pyridin-2-yl)-5-isopropyl-7H-pyrrolo[2,3-d]pyrimidin-4-yl)-2,5-dimethylpiperazine-1-carboxylate

To a solution of tert-butyl (2R,5S)-4-(7-(4-(ethoxycarbonyl)pyridin-2-yl)-5-(prop-1-en-2-yl)-7H-pyrrolo[2,3-d]pyrimidin-4-yl)-2,5-dimethylpiperazine-1-carboxylate (180 mg, 0.35 mmol) in MeOH (15 mL) was added palladium (100 mg, 10% on carbon, wetted with ca. 55% water). The resulting mixture was stirred under H₂ atmosphere (˜0.1 MPa) overnight. The reaction mixture was then filtered and concentrated. The residue was used in the next step directly. LC/MS ESI (m/z): 523 (M+H)⁺.

### Step 5. 2-(4-((2S,5R)-4-(tert-Butoxycarbonyl)-2,5-dimethylpiperazin-1-yl)-5-isopropyl-7H-pyrrolo[2,3-d]pyrimidin-7-yl)isonicotinic acid

To a solution of tert-butyl (2R,5S)-4-(7-(4-(ethoxycarbonyl)pyridin-2-yl)-5-isopropyl-7H-pyrrolo[2,3-d]pyrimidin-4-yl)-2,5-dimethylpiperazine-1-carboxylate (180 mg, 0.34 mmol) in MeOH (5 mL) and water (4 mL) was added NaOH (27 mg, 0.68 mmol). After 2 h, the resulting mixture was acidified by 1 N HCl to pH 6 and extracted twice with EtOAc. The combined organic layers were dried over Na₂SO₄, filtered and concentrated. The residue was used in the next step directly. LC/MS ESI (m/z): 495 (M+H)⁺.

### Step 6. tert-Butyl (2R,5S)-4-(7-(4-carbamoylpyridin-2-yl)-5-isopropyl-7H-pyrrolo[2,3-d]pyrimidin-4-yl)-2,5-dimethylpiperazine-1-carboxylate

To a solution of 2-(4-((2S,5R)-4-(tert-butoxycarbonyl)-2,5-dimethylpiperazin-1-yl)-5-isopropyl-7H-pyrrolo[2,3-d]pyrimidin-7-yl)isonicotinic acid (140 mg, 0.28 mmol) in DMF (5 mL) were added NH₄Cl (75 mg, 1.4 mmol), EDCI (81 mg, 0.42 mmol) and DIEA (0.39 mL, 2.2 mmol). The resulting mixture was stirred at room temperature overnight. The reaction was partitioned between EtOAc and water. The aqueous layer was extracted twice with EtOAc. The combined organic layers were washed brine, dried over Na₂SO₄, filtered and concentrated. The residue was purified by flash column chromatography (silica gel, 0˜100% ethyl acetate in petroleum ether) to afford tert-butyl (2R,5S)-4-(7-(4-carbamoylpyridin-2-yl)-5-isopropyl-7H-pyrrolo[2,3-d]pyrimidin-4-yl)-2,5-dimethylpiperazine-1-carboxylate (110 mg, 79%). LC/MS ESI (m/z): 494 (M+H)⁺.

### Step 7. tert-Butyl (2R,5S)-4-(7-(4-cyanopyridin-2-yl)-5-isopropyl-7H-pyrrolo[2,3-d]pyrimidin-4-yl)-2,5-dimethylpiperazine-1-carboxylate

To a solution of tert-butyl (2R,5S)-4-(7-(4-carbamoylpyridin-2-yl)-5-isopropyl-7H-pyrrolo[2,3-d]pyrimidin-4-yl)-2,5-dimethylpiperazine-1-carboxylate (110 mg, 0.22 mmol) in DCM (5 mL) at 0° C. were added DIEA (0.08 mL, 0.45 mmol), followed by TFAA (70 mg, 0.33 mmol). After 3 h, the reaction was quenched with ice water. The layers were separated, and the aqueous layer was extracted twice with DCM. The combined organic layers were washed with brine, dried over Na₂SO₄, filtered and concentrated. Purification by prep-HPLC afforded tert-butyl (2R,5S)-4-(7-(4-cyanopyridin-2-yl)-5-isopropyl-7H-pyrrolo[2,3-d]pyrimidin-4-yl)-2,5-dimethylpiperazine-1-carboxylate (41 mg, 39%) as a white solid. LC/MS ESI (m/z): 476 (M+H)⁺. ¹H NMR (400 MHz, CDCl₃) δ 9.27 (d, J=1.0 Hz, 1H), 8.54 (dd, J=5.0, 0.8 Hz, 1H), 8.45 (m, 1H), 7.91 (m, 1H), 7.28 (dd, J=5.0, 1.3 Hz, 1H), 4.46-4.21 (m, 2H), 3.76-3.66 (m, 2H), 3.58 (m, 1H), 3.36 (m, 1H), 3.19-3.02 (m, 1H), 1.43 (s, 9H), 1.39 (d, J=6.6 Hz, 3H), 1.21 (d, J=6.7 Hz, 3H), 1.10-1.04 (m, 6H).

### Example 207. Synthesis of tert-butyl (2R,5S)-4-(1-(4-fluoropyridin-2-yl)-3-methyl-1H-pyrrolo[3,2-c]pyridin-4-yl)-2,5-dimethylpiperazine-1-carboxylate (Compound 701)

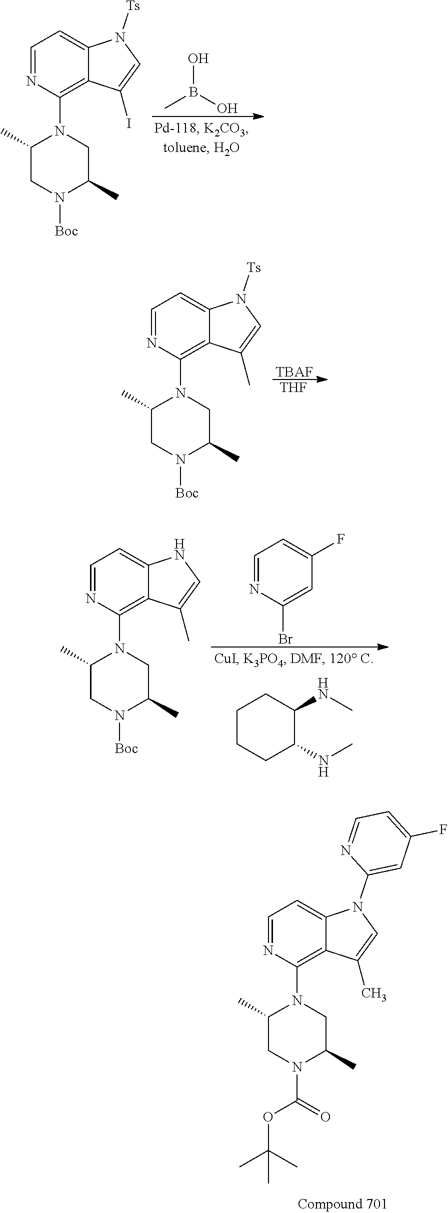

### Step 1. tert-Butyl (2R,5S)-2,5-dimethyl-4-(3-methyl-1-tosyl-1H-pyrrolo[3,2-c]pyridin-4-yl)piperazine-1-carboxylate

To a mixture of tert-butyl (2R,5S)-4-[3-iodo-1-(4-methylbenzenesulfonyl)-1H-pyrrolo[3,2-c]pyridin-4-yl]-2,5-dimethylpiperazine-1-carboxylate (2.4 g, 3.9 mmol, prepared following the procedure outlined for compound 620) in toluene (50 mL) and water (3 mL) were added methylboronic acid (0.47 g, 7.9 mmol) and K₂CO₃ (2.7 g, 20 mmol) and PdCl₂(dtbpf) (0.32 g, 0.39 mmol). The resulting mixture was stirred at 100° C. for 3 h. After cooling to room temperature, the reaction mixture was partitioned between NH₄Cl (aq.) and EtOAc, and the aqueous layer was extracted twice with EtOAc. The combined organic layers were washed with brine, dried over Na₂SO₄, filtered and concentrated. The residue was purified by flash column chromatography (silica gel, 0-40% ethyl acetate in petroleum ether) to afford tert-butyl (2R,5S)-2,5-dimethyl-4-(3-methyl-1-tosyl-1H-pyrrolo[3,2-c]pyridin-4-yl)piperazine-1-carboxylate (1.3 g, 66%) as a white solid. LC/MS ESI (m/z): 499 (M+H)⁺.

### Step 2. tert-Butyl (2R,5S)-2,5-dimethyl-4-(3-methyl-1H-pyrrolo[3,2-c]pyridin-4-yl)piperazine-1-carboxylate

To a solution of tert-butyl (2R,5S)-2,5-dimethyl-4-(3-methyl-1-tosyl-1H-pyrrolo[3,2-c]pyridin-4-yl)piperazine-1-carboxylate (1.3 g, 2.6 mmol) in THE (2 mL) was added TBAF (3.9 mL, 1.0 M in THF) at room temperature. The mixture was stirred at room temperature overnight. The reaction was poured into water and extracted twice with EtOAc. The combined organic layers were washed with brine, dried over Na₂SO₄, filtered and concentrated. The residue was purified by flash column chromatography (silica gel, 0-80% ethyl acetate in petroleum ether) to afford tert-butyl (2R,5S)-2,5-dimethyl-4-(3-methyl-1H-pyrrolo[3,2-c]pyridin-4-yl)piperazine-1-carboxylate (700 mg, 78%) as a white solid. LC/MS ESI (m/z): 345 (M+H)⁺.

### Step 3. tert-Butyl (2R,5S)-4-(1-(4-fluoropyridin-2-yl)-3-methyl-1H-pyrrolo[3,2-c]pyridin-4-yl)-2,5-dimethylpiperazine-1-carboxylate

A mixture of tert-butyl (2R,5S)-2,5-dimethyl-4-(3-methyl-1H-pyrrolo[3,2-c]pyridin-4-yl)piperazine-1-carboxylate (100 mg, 0.32 mmol), 2-bromo-4-fluoropyridine (62 mg, 0.35 mmol), CuI (60 mg, 0.30 mmol) and K₃PO₄ (200 mg, 0.95 mmol) and trans-N¹,N²-dimethylcyclohexane-1,2-diamine (14 mg, 0.10 mmol) in DMF (2 mL) was stirred at 80° C. for 3 h. After cooling to room temperature, the reaction was poured into water and extracted twice with EtOAc. The combined organic layers were washed with brine, dried over Na₂SO₄, filtered and concentrated. The residue was purified by flash column chromatography (silica gel, 0˜40% ethyl acetate in petroleum ether) to afford tert-butyl (2R,5S)-4-(1-(4-fluoropyridin-2-yl)-3-methyl-1H-pyrrolo[3,2-c]pyridin-4-yl)-2,5-dimethylpiperazine-1-carboxylate (17 mg, 13%) as a yellow solid. LC/MS ESI (m/z): 440 (M+H)⁺. ¹H NMR (400 MHz, DMSO-d6) δ 8.60 (dd, J=9.2, 5.7 Hz, 1H), 8.07-8.02 (m, 1H), 7.96 (d, J=5.9 Hz, 1H), 7.87 (s, 1H), 7.72 (d, J=10.1 Hz, 1H), 7.31-7.24 (d, 1H), 4.37-4.31 (d, 1H), 3.94-3.88 (m, 1H), 3.69 (d, J=12.9 Hz, 1H), 3.61-3.54 (m, 2H), 2.98 (d, J=12.1 Hz, 1H), 2.52 (s, 3H), 1.43 (s, 9H), 1.22 (d, J=6.7 Hz, 3H), 0.83 (d, J=6.3 Hz, 3H).

The following compounds were prepared by procedures analogous to the synthesis of compound 701 from corresponding chiral amines and boronic acids.

| Cmpd | | |
|---|---|---|
| No. | Chemical Name | LCMS and ¹H NMR |
| 702 | tert-butyl (2R,5S)-2,5-dimethyl- | LC/MS ESI (m/z): 490 (M + H)⁺. ¹H NMR (400 MHz, |
| | 4-(3-methyl-1-(4- | DMSO-d6) δ 8.83 (d, J = 5.1 Hz, 1H), 8.09 (s, 1H), 8.06-8.01 |
| | (trifluoromethyl)pyridin-2-yl)- | (m, 2H), 7.99 (d, J = 5.9 Hz, 1H), 7.67 (d, J = 5.1 Hz, |
| | 1H-pyrrolo[3,2-c]pyridin-4- | 1H), 4.37-4.29 (m, 1H), 3.96-3.87 (m, 1H), 3.69 (d, J = |
| | yl)piperazine-1-carboxylate | 11.7 Hz, 1H), 3.63-3.51 (m, 2H), 2.98 (d, J = 12.3 Hz, |
| | | 1H), 2.52 (s, 3H), 1.44 (s, 9H), 1.22 (d, J = 6.7 Hz, 3H), |
| | | 0.83 (d, J = 6.5 Hz, 3H). |
| 703 | tert-butyl (S)-4-(3-(2- | LC/MS ESI (m/z): 505 (M + H)⁺. ¹H NMR (400 MHz, |
| | fluorophenyl)-1-(3- | CDCl₃) δ 8.11 (d, J = 5.9 Hz, 1H), 7.57-7.48 (m, 2H), |
| | fluorophenyl)-1H-pyrrolo[3,2- | 7.40-7.30 (m, 3H), 7.25-7.23 (m, 1H), 7.23-7.09 (m, |
| | c]pyridin-4-yl)-3- | 4H), 3.58-3.44 (m, 1H), 3.16-2.82 (m, 6H), 1.42 (s, 9H), |
| | methylpiperazine-1-carboxylate | 0.93 (d, J = 4.1 Hz, 3H). |
| 704 | tert-butyl (R)-4-(3-cyclopropyl- | LC/MS ESI (m/z): 451 (M + H)⁺. ¹H NMR (400 MHz, |
| | 1-(3-fluorophenyl)-1H- | CDCl₃) δ 7.91 (d, J = 5.9 Hz, 1H), 7.43-7.36 (m, 1H), |
| | pyrrolo[3,2-c]pyridin-4-yl)-2- | 7.17-7.12 (m, 1H), 7.10-7.05 (m, 1H), 7.03-6.95 (m, |
| | methylpiperazine-1-carboxylate | 2H), 6.77 (s, 1H), 4.43-4.32 (m, 1H), 4.01-3.85 (m, 2H), |
| | | 3.50-3.44 (m, 1H), 3.36-3.27 (m, 1H), 3.25-3.17 (m, |
| | | 1H), 2.78-2.68 (m, 1H), 2.37-2.29 (m, 1H), 1.43 (s, 9H), |
| | | 1.25 (d, J = 8.0 Hz 3H), 1.01-0.93 (m, 2H), 0.83-0.78 |
| | | (m, 1H), 0.57-0.49 (m, 1H). |

### Example 208. Synthesis of tert-butyl (2R,5S)-4-(1-(4-cyanopyridin-2-yl)-3-methyl-11H-pyrrolo[3,2-c]pyridin-4-yl)-2,5-dimethylpiperazine-1-carboxylate (Compound 705)

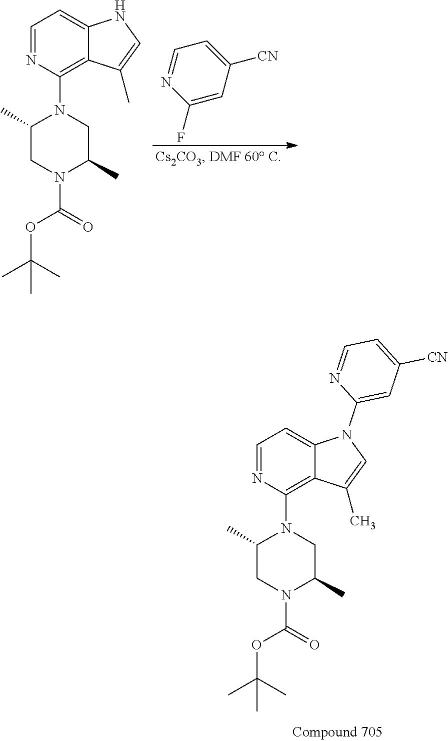

To a solution of tert-butyl (2R,5S)-2,5-dimethyl-4-(3-methyl-1H-pyrrolo[3,2-c]pyridin-4-yl)piperazine-1-carboxylate (100 mg, 0.29 mmol, prepared following the procedure outlined for compound 701) in DMF (6 mL) were added 2-fluoropyridine-4-carbonitrile (97 mg, 0.79 mmol) and Cs₂CO₃ (520 mg, 1.6 mmol). The resulting mixture was stirred at 60° C. overnight. After cooling to room temperature, the reaction was poured into water and EtOAc. The aqueous layer was extracted twice with EtOAc. The combined organic layers were washed with brine, dried over Na₂SO₄, filtered and concentrated. The residue was purified by flash column chromatography (silica gel, 0-40% ethyl acetate in petroleum ether) to afford tert-butyl (2R,5S)-4-(1-(4-cyanopyridin-2-yl)-3-methyl-1H-pyrrolo[3,2-c]pyridin-4-yl)-2,5-dimethylpiperazine-1-carboxylate (80 mg, 60%) as a light yellow solid. LC/MS ESI (m/z): 447 (M+H)⁺. ¹H NMR (400 MHz, DMSO-d6) δ 8.79 (d, J=5.0 Hz, 1H), 8.27 (s, 1H), 8.03 (d, J=5.9 Hz, 1H), 7.98 (d, J=5.9 Hz, 1H), 7.92 (s, 1H), 7.74 (dd, J=5.0, 1.1 Hz, 1H), 4.38-4.29 (m, 1H), 3.95-3.87 (m, 1H), 3.68 (d, J=11.5 Hz, 1H), 3.62-3.52 (m, 2H), 2.98 (d, J=12.5 Hz, 1H), 2.53 (s, 3H), 1.43 (s, 9H), 1.22 (d, J=6.7 Hz, 3H), 0.83 (d, J=6.5 Hz, 3H).

### Example 209. Synthesis of tert-butyl (S)-4-(3-cyclobutyl-1-(3-fluorophenyl)-1H-pyrrolo[3,2-c]pyridin-4-yl)-3-methylpiperazine-1-carboxylate (Compound 706)

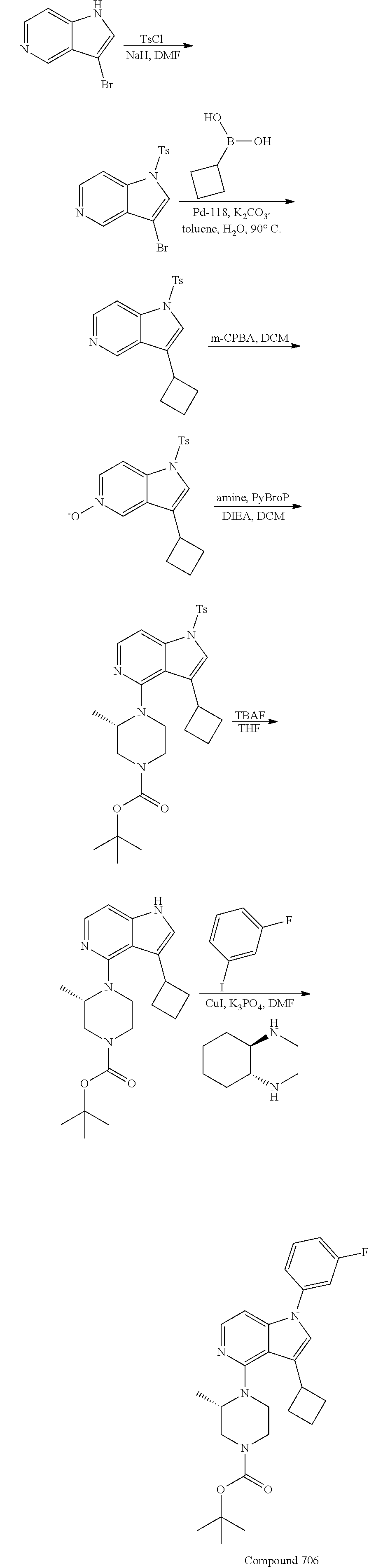

### Step 1. 3-Bromo-1-tosyl-1H-pyrrolo[3,2-c]pyridine

To a suspension of NaH (120 mg, 3.1 mmol, 60 wt % in mineral oil) in anhydrous DMF (10 mL) at 0° C. were added 3-bromo-1H-pyrrolo[3,2-c]pyridine (400 mg, 2.0 mmol) in portions, followed by 4-methylbenzenesulfonyl chloride (580 mg, 3.1 mmol) in portions. After 40 min, the reaction was poured into ice water and extracted twice with EtOAc. The combined organic layers were washed with brine, dried over Na₂SO₄, filtered and concentrated. The residue was purified by flash column chromatography (silica gel, 0-40% ethyl acetate in petroleum ether) to afford 3-bromo-1-tosyl-1H-pyrrolo[3,2-c]pyridine (650 mg, 91%) as a light yellow solid. LC/MS ESI (m/z): 351, 353 (M+H)⁺.

### Step 2. 3-Bromo-1-tosyl-1H-pyrrolo[3,2-c]pyridine

To a solution of 3-bromo-1-tosyl-1H-pyrrolo[3,2-c]pyridine (600 mg, 1.7 mmol) in toluene (10 mL) and H₂O (1 mL) were added cyclobutylboronic acid (340 mg, 3.4 mmol), K₂CO₃ (1.1 g, 8.5 mmol) and Pd(dptf)Cl₂ (110 mg, 0.17 mmol). The resulting mixture was stirred at 90° C. overnight. After cooling to room temperature, the reaction was concentrated and the residue was purified by flash column chromatography (silica gel, 0˜30%, ethyl acetate in petroleum ether) to afford 3-cyclobutyl-1-tosyl-1H-pyrrolo[3,2-c]pyridine (220 mg, 39%) as a yellow solid. LC/MS ESI (m/z): 327 (M+H)⁺.

### Step 3. 3-Cyclobutyl-1-tosyl-1H-pyrrolo[3,2-c]pyridine 5-oxide

To a solution of 3-cyclobutyl-1-tosyl-1H-pyrrolo[3,2-c]pyridine (200 mg, 0.61 mmol) in DCM (10 m1) at 0° C. was added 3-chlorobenzoperoxoic acid (210 mg, 1.2 mmol) in portions. The resulting mixture was stirred at room temperature under N₂ overnight. The reaction was quenched with H₂O and extracted twice with DCM. The combined organic layers were washed with brine, dried over Na₂SO₄, filtered and concentrated. The residue was purified by flash column chromatography (silica gel, 0˜20% MeOH in DCM) to afford 3-cyclobutyl-1-tosyl-1H-pyrrolo[3,2-c]pyridine 5-oxide (140 mg, 67%) as a yellow solid. LC/MS ESI (m/z): 343 (M+H)⁺.

### Step 4. tert-Butyl (S)-4-(3-cyclobutyl-1-tosyl-1H-pyrrolo[3,2-c]pyridin-4-yl)-3-methylpiperazine-1-carboxylate

To a solution of 3-cyclobutyl-1-tosyl-1H-pyrrolo[3,2-c]pyridine 5-oxide (140 mg, 0.41 mmol), tert-butyl (S)-3-methylpiperazine-1-carboxylate (410 mg, 2.0 mmol) and DIPEA (200 mg, 1.5 mmol) in DCM (10 mL) was added PyBrOP (290 mg, 0.60 mmol). The resulting mixture was stirred at room temperature under N₂ overnight. The reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜40% ethyl acetate in petroleum ether) to afford tert-butyl (S)-4-(3-cyclobutyl-1-tosyl-1H-pyrrolo[3,2-c]pyridin-4-yl)-3-methylpiperazine-1-carboxylate (60 mg, 28% yield) as a withe solid. LC/MS ESI (m/z): 525 (M+H)⁺.

### Step 5. tert-Butyl (S)-4-(3-cyclobutyl-1H-pyrrolo[3,2-c]pyridin-4-yl)-3-methylpiperazine-1-carboxylate

To a solution of tert-butyl (S)-4-(3-cyclobutyl-1-tosyl-1H-pyrrolo[3,2-c]pyridin-4-yl)-3-methylpiperazine-1-carboxylate (60 mg, 0.12 mmol) in THE (5 mL) was added TBAF (1.0 mL, 1.0 M in THF). After 2 h, the reaction was partitioned between EtOAc and brine. The aqueous layer was extracted twice with EtOAc. The combined organic layers were washed with brine, dried over Na₂SO₄, filtered and concentrated. The residue was used in the next step directly. LC/MS ESI (m/z): 371 (M+H)⁺.

### Step 6. tert-Butyl (S)-4-(3-cyclobutyl-1-(3-fluorophenyl)-1H-pyrrolo[3,2-c]pyridin-4-yl)-3-methylpiperazine-1-carboxylate

To a solution of tert-butyl (S)-4-(3-cyclobutyl-1H-pyrrolo[3,2-c]pyridin-4-yl)-3-methylpiperazine-1-carboxylate (25 mg, 0.07 mmol) in DMF (5 mL) were added 1-fluoro-3-iodobenzene (45 mg, 0.20 mmol), trans-N,N′-dimethylcyclohexane-1,2-diamine (5.0 mg, 0.04 mmol), CuI (7.0 mg, 0.04 mmol) and K₃PO₄ (43 mg, 0.20 mmol). The resulting mixture was heated to 120° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0˜50% ethyl acetate in petroleum ether) to afford tert-butyl (S)-4-(3-cyclobutyl-1-(3-fluorophenyl)-1H-pyrrolo[3,2-c]pyridin-4-yl)-3-methylpiperazine-1-carboxylate (9.6 mg, 31%) as a white solid. LC/MS ESI (m/z): 465 (M+H)⁺. ¹H NMR (400 MHz, CDCl₃) δ 8.06 (d, J=5.8 Hz, 1H), 7.52-7.46 (m, 1H), 7.30-7.27 (m, 1H), 7.23-7.16 (m, 3H), 7.12-7.06 (m, 1H), 4.09-4.01 (m, 1H), 3.91 (s, 1H), 3.81 (s, 1H), 3.68-3.61 (m, 1H), 3.51 (s, 1H), 3.27 (d, J=11.2 Hz, 1H), 3.19-3.11 (m, 1H), 3.03-2.95 (m, 1H), 2.61-2.53 (m, 1H), 2.46-2.38 (m, 1H), 2.22-2.14 (m, 1H), 2.10-2.01 (m, 2H), 1.93 (m, 1H), 1.50 (s, 9H), 0.96 (d, J=6.2 Hz, 3H).

### Example 210. Synthesis of tert-butyl (2R,5S)-4-(7-(4-cyanopyridin-2-yl)-5-(trifluoromethyl)-7H-pyrrolo[2,3-d]pyrimidin-4-yl)-5-methyl-2-(methyl-d₃)piperazine-1-carboxylate (Compound 707)

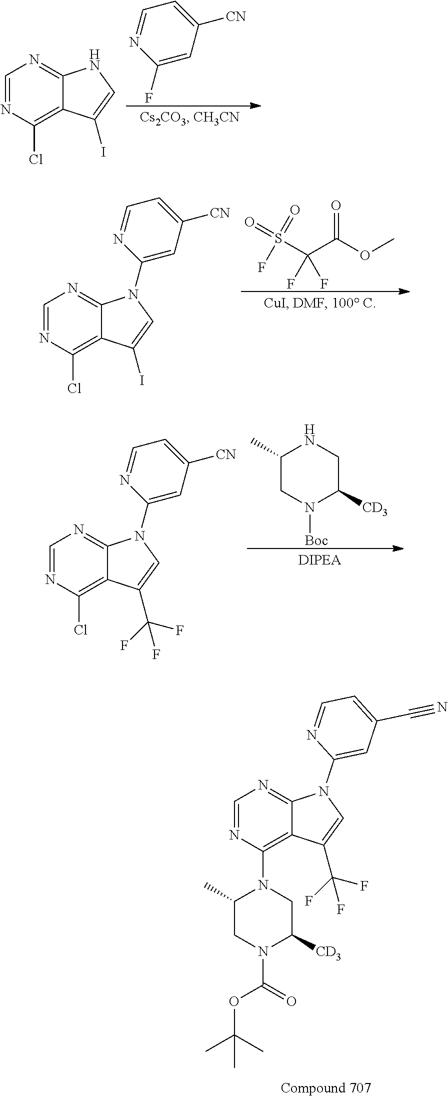

### Step 1. 2-(4-Chloro-5-iodo-7H-pyrrolo[2,3-d]pyrimidin-7-yl)isonicotinonitrile

To a solution of 4-chloro-5-iodo-7H-pyrrolo[2,3-d]pyrimidine (7.0 g, 25 mmol) and 2-fluoroisonicotinonitrile (18 g, 150 mmol) in CH₃CN (70 mL) was added Cs₂CO₃ (12 g, 38 mmol). The resulting mixture was stirred at 25° C. under N₂ for 72 h. The crude product was obtained by filtration and washed with water and CH₃CN to afford 2-(4-chloro-5-iodo-7H-pyrrolo[2,3-d]pyrimidin-7-yl)isonicotinonitrile (9.0 g, 94%) as a light brown solid. LC/MS ESI (m/z): 382 (M+H)⁺.

### Step 2. 2-(4-Chloro-5-(trifluoromethyl)-7H-pyrrolo[2,3-d]pyrimidin-7-yl)isonicotinonitrile

To a mixture of 2-(4-chloro-5-iodo-7H-pyrrolo[2,3-d]pyrimidin-7-yl)isonicotinonitrile (1.4 g, 3.7 mmol) and CuI (720 mg, 3.8 mmol) in DMF (15 mL) was added methyl 2,2-difluoro-2-(fluorosulfonyl)acetate (2.9 g, 15 mmol) under N₂. The resulting mixture was stirred at 100° C. under N₂ overnight. The reaction was partitioned between EtOAc and brine. The aqueous layer was extracted twice with EtOAc. The combined organic layers were washed with brine, dried over Na₂SO₄, filtered and concentrated. The residue was purified through flash column chromatography (silica gel, 0˜50%, ethyl acetate in petroleum ether) to afford 2-(4-chloro-5-(trifluoromethyl)-7H-pyrrolo[2,3-d]pyrimidin-7-yl)isonicotinonitrile (670 mg, 80% purity, 1.7 mmol, 44% yield) as a white solid. LC/MS ESI (m/z): 324 (M+H)⁺.

### Step 3. tert-Butyl (2R,5S)-4-(7-(4-cyanopyridin-2-yl)-5-(trifluoromethyl)-7H-pyrrolo[2,3-d]pyrimidin-4-yl)-5-methyl-2-(methyl-d₃)piperazine-1-carboxylate

To a solution of tert-butyl (2R,5S)-5-methyl-2-(methyl-d₃)piperazine-1-carboxylate (30 mg, 0.13 mmol, prepared following the procedure outlined for compound 709) in DIPEA (2 mL) was added 2-(4-chloro-5-(trifluoromethyl)-7H-pyrrolo[2,3-d]pyrimidin-7-yl)isonicotinonitrile (89 mg, 0.27 mmol). The resulting mixture was stirred at 120° C. for 2 h. After cooling to room temperature, the reaction was concentrated and the residue was purified by flash column chromatography (silica gel, 0˜20%, ethyl acetate in petroleum ether) to afford tert-butyl (2R,5S)-4-(7-(4-cyanopyridin-2-yl)-5-(trifluoromethyl)-7H-pyrrolo[2,3-d]pyrimidin-4-yl)-5-methyl-2-(methyl-d₃)piperazine-1-carboxylate (30 mg, 43%) as a yellow oil. It was purified by prep-HPLC to afford 5.8 mg of title compound as a white solid. LC/MS ESI (m/z): 505 (M+H)⁺. ¹H NMR (400 MHz, CD₃OD) δ 9.23-9.12 (m, 1H), 8.76 (m, 2H), 8.59 (s, 1H), 7.70 (dd, J=5.0, 1.3 Hz, 1H), 4.62-4.54 (m, 1H), 4.34 (s, 1H), 3.80 (dd, J=13.6, 4.1 Hz, 1H), 3.75 (dd, J=13.3, 2.8 Hz, 1H), 3.64 (m, 1H), 3.51 (d, J=13.6 Hz, 1H), 1.50 (s, 9H), 1.14 (d, J=6.6 Hz, 3H).

The following compound was prepared by a procedure analogous to the synthesis of compound 707 from the corresponding amine (EtOH as solvent, DIEA as base, 100° C.).

| Cmpd | | |
|---|---|---|
| No. | Chemical Name | LCMS and ¹H NMR |
| 708 | tert-butyl 4-(7-(4-cyanopyridin- | LC/MS ESI (m/z): 474 (M + H)⁺. ¹H NMR(400 MHz, |
| | 2-yl)-5-(trifluoromethyl)-7H- | CDCl₃) δ 9.34 (s, 1H), 8.68-8.66 (m, 2H), 8.60 (s, 1H), |
| | pyrrolo[2,3-d]pyrimidin-4- | 7.49 (dd, J = 4.9, 0.9 Hz, 1H), 3.62-3.59 (m, 4H), 3.57-3.54 |
| | yl)piperazine-1-carboxylate | (m, 4H), 1.50 (s, 9H). |

### Example 211. Synthesis of tert-butyl (2R,5S)-4-(7-(4-cyanopyridin-2-yl)-5-(2-fluorophenyl)-5H-pyrrolo[3,2-d]pyrimidin-4-yl)-5-methyl-2-(methyl-d₃)piperazine-1-carboxylate (Compound 709)

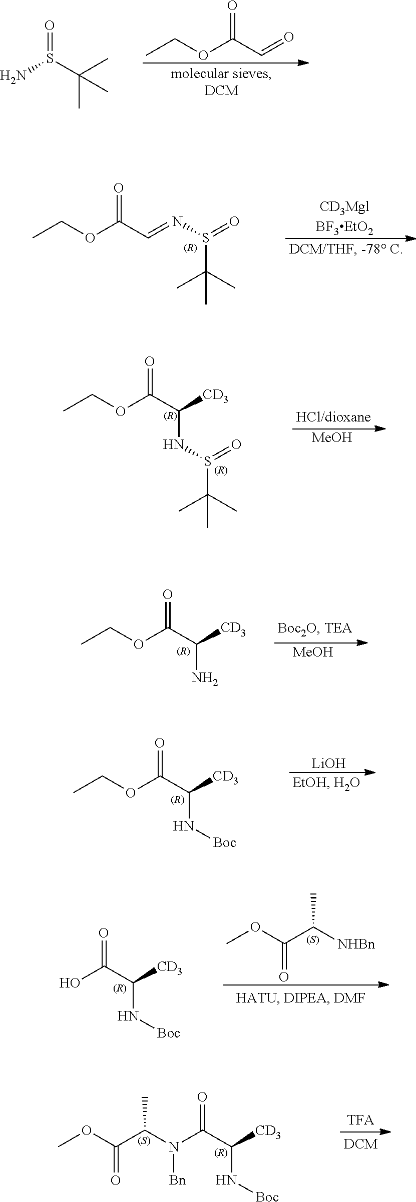

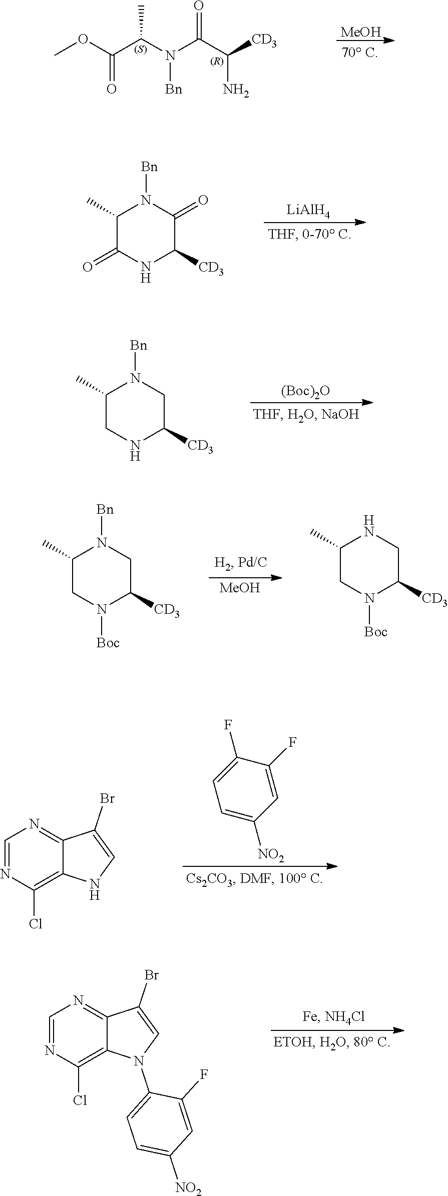

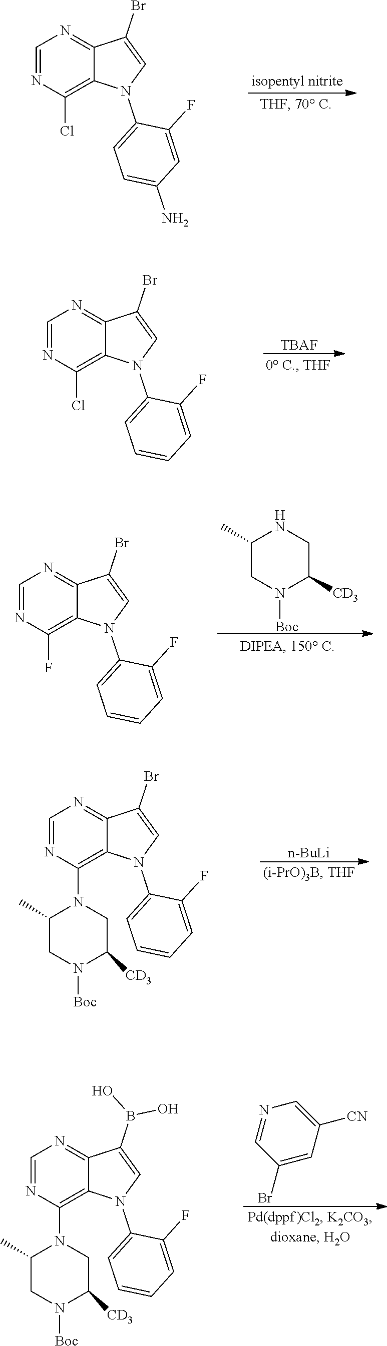

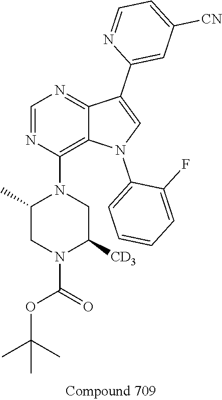 [SMILES: [2H]C([2H])([2H])[C@@H]1CN(c2ncnc3c(-c4cc(C#N)ccn4)cn(-c4ccccc4F)c23)[C@@H](C)CN1C(=O)OC(C)(C)C]

### Step 1. Ethyl (R,E)-2-((tert-butylsulfinyl)imino)acetate

To a solution of ethyl 2-oxoacetate (6.4 g, 63 mmol) in CH₂Cl₂ (300 mL) were added (R)-2-methylpropane-2-sulfinamide (7.6 g, 63 mmol) and 4 A molecular sieves (20 g). After 72 h, the mixture was filtered through a bed of Celite and washed with EtOAc. The filtrate was dried over Na₂SO₄, filtered and concentrated. The residue was purified with flash column chromatography (silica gel, 0˜20% ethyl acetate in petroleum ether) to afford ethyl (R,E)-2-((tert-butylsulfinyl)imino)acetate (7.2 g, 56%) as a colorless oil.

### Step 2. Ethyl ((R)-tert-butylsulfinyl)-D-alaninate-3,3,3-d₃

To a solution of ethyl (R,E)-2-((tert-butylsulfinyl)imino)acetate (6.2 g, 30 mmol) in DCM (400 mL) and THF (120 mL) at −78° C. was added BF₃·Et₂O (7.4 mL, 60 mmol) dropwise under N₂. The resulting mixture was stirred at −78° C. for 5 min and treated with CD₃MgI (1.0 M in Et₂O, 60 mL, 60 mmol) dropwise. After stirring at −78° C. for 10 min, the reaction was quenched with NaHCO₃ (aq.) and partitioned between EtOAc and brine. The aqueous layer was extracted twice with EtOAc. The combined organic layers were washed with brine, dried over Na₂SO₄, filtered and concentrated. The residue was purified by flash column chromatography (silica gel, 0˜50%, ethyl acetate in petroleum ether) to afford ethyl ((R)-tert-butylsulfinyl)-D-alaninate-3,3,3-d₃ (1.4 g, 21% yield, dr=97/3) as a white solid. LC/MS ESI (m/z): 225 (M+H)⁺. The configuration was verified by ¹H NMR (Reference: J. Org. Chem. 1999, 64, 3396-3397).

### Step 3. Ethyl D-alaninate-3,3,3-d₃

To a solution of ethyl ((R)-tert-butylsulfinyl)-D-alaninate-3,3,3-d₃ (1.4 g, 6.2 mmol) in MeOH (30 mL) were added HCl (5.0 mL, 4.0 M in dioxane). The resulting mixture was stirred at room temperature under N₂ for 2 h. The solvent of reaction was removed under vacuum to afford ethyl D-alaninate-3,3,3-d₃, as a white solid (1.0 g crude HCl salt). LC/MS ESI (m/z): 121 (M+H)⁺.

### Step 4. Ethyl (tert-butoxycarbonyl)-D-alaninate-3,3,3-d₃

To a solution of ethyl D-alaninate-3,3,3-d₃ (1.0 g crude HCl salt) in MeOH (30 mL) were added TEA (1.7 mL, 12 mmol) and Boc₂O (1.5 g, 6.9 mmol). The resulting mixture was stirred at room temperature under N₂ for 2 h. The reaction was partitioned between EtOAc and NH₄Cl (aq.). The aqueous layer was extracted twice with EtOAc. The combined organic layers were washed with brine, dried over Na₂SO₄, filtered, and concentrated to afford crude product ethyl (tert-butoxycarbonyl)-D-alaninate-3,3,3-d₃ (1.4 g crude) as a white solid. LC/MS ESI (m/z): 221 (M+H)⁺.

### Step 5. (tert-Butoxycarbonyl)-D-alanine-3,3,3-d₃

To a solution of ethyl (tert-butoxycarbonyl)-D-alaninate-3,3,3-d₃ (1.4 g crude) in EtOH (20 mL) and H₂O (10 mL) in an ice bath was added LiOH (310 mg, 13 mmol). The resulting mixture was stirred at room temperature under N₂ for 1 h. The reaction was partitioned between EtOAc and brine. The aqueous layer was acidified by HCl (1.0 M) to pH 3 and extracted twice with EtOAc. The organic layer was washed with brine, dried over Na₂SO₄, filtered and concentrated to afford (tert-butoxycarbonyl)-D-alanine-3,3,3-d₃ (900 mg, 75% yield over three steps) LC/MS ESI (m/z): 193 (M+H)⁺.

### Step 6. Methyl N-benzyl-N-((tert-butoxycarbonyl)-D-alanyl-3,3,3-d₃)-L-alaninate

To a solution of (tert-butoxycarbonyl)-D-alanine-3,3,3-d₃ (900 mg, 4.7 mmol) and methyl benzyl-L-alaninate hydrochloride (1.2 g, 5.2 mmol) in DMF (20 mL) were added DIPEA (2.4 g, 19 mmol) and HATU (2.7 g, 7.0 mmol). The resulting mixture was stirred at room temperature under N₂ overnight. The reaction was partitioned between EtOAc and aqueous NaHCO₃. The aqueous layer was extracted twice with EtOAc. The combined organic layers were washed with brine, dried over Na₂SO₄, filtered and concentrated. The residue was purified by flash column chromatography (silica gel, 0˜60%, ethyl acetate in petroleum ether) to afford methyl N-benzyl-N-((tert-butoxycarbonyl)-D-alanyl-3,3,3-d₃)-L-alaninate (1.4 g, 81% yield) as a yellow solid. LC/MS ESI (m/z): 368 (M+H)⁺.

### Step 7. Methyl N-(D-alanyl-3,3,3-d₃)—N-benzyl-L-alaninate

To a solution of methyl N-benzyl-N-((tert-butoxycarbonyl)-D-alanyl-3,3,3-d₃)-L-alaninate (1.4 g, 3.8 mmol) in DCM (20 mL) at 0° C. was added TFA (10 mL). The resulting mixture was stirred at room temperature under N₂ for 3 h. The solvent of the reaction was removed under vacuum to afford crude product methyl N-(D-alanyl-3,3,3-d₃)—N-benzyl-L-alaninate (1.5 g crude TFA salt) as a yellow solid. LC/MS ESI (m/z): 268 (M+H)⁺.

### Step 8. (3R,6S)-1-Benzyl-6-methyl-3-(methyl-d₃)piperazine-2,5-dione

A solution of methyl N-(D-alanyl-3,3,3-d₃)—N-benzyl-L-alaninate (1.5 g crude TFA salt) in MeOH (10 mL) was heated at 70° C. under N₂ for 4 h. The reaction mixture was then partitioned between EtOAc and brine aqueous. The aqueous layer was extracted twice with EtOAc. The combined organic layers were washed with brine, dried over Na₂SO₄, filtered, and concentrated to afford (3R,6S)-1-benzyl-6-methyl-3-(methyl-d₃)piperazine-2,5-dione (1.0 g crude) as a yellow solid. LC/MS ESI (m/z): 236 (M+H)⁺.

### Step 9. tert-Butyl (2R,5S)-4-benzyl-5-methyl-2-(methyl-d₃)piperazine-1-carboxylate

A solution of (3R,6S)-1-benzyl-6-methyl-3-(methyl-d₃)piperazine-2,5-dione (1.0 g crude) in THF (10 mL) in an ice bath under N₂ was treated with LiAlH₄ (1.0 M in THF, 20 mL) dropwise. The reaction mixture was stirred at 70° C. for 3 h. The reaction mixture was carefully quenched with 20% NaOH (20 mL) and the resulting mixture was used in the next step directly. LC/MS ESI (m/z): 208 (M+H)⁺. The mixture was taken up in THE-water and was then treated with Boc₂O (1.7 g, 7.6 mmol). After stirring at room temperature overnight, the reaction mixture was filtered. The residue was purified by flash column chromatography (silica gel, 0-10%, ethyl acetate in petroleum ether) to afford tert-butyl (2R,5S)-4-benzyl-5-methyl-2-(methyl-d₃)piperazine-1-carboxylate (990 mg, 85% yield dr=90/10) as a colorless oil. LC/MS ESI (m/z): 308 (M+H)⁺. ¹H NMR (400 MHz, CDCl₃) δ 7.37-7.28 (m, 4H), 7.26-7.21 (m, 1H), 4.17 (d, J=3.9 Hz, 1H), 3.69-3.57 (m, 2H), 3.46 (d, J=13.6 Hz, 1H), 3.30 (dd, J=13.0, 3.7 Hz, 1H), 2.92 (dd, J=10.9, 5.9 Hz, 1H), 2.69 (dd, J=11.7, 4.4 Hz, 1H), 2.19 (dd, J=11.7, 1.9 Hz, 1H), 1.46 (s, 9H), 0.98 (d, J=6.5 Hz, 3H).

### Step 10. tert-Butyl (2R,5S)-5-methyl-2-(methyl-d3)piperazine-1-carboxylate

To a solution of tert-butyl (2R,5S)-4-benzyl-5-methyl-2-(methyl-d₃)piperazine-1-carboxylate (550 mg, 1.7 mmol) in MeOH (15 mL) was added Pd (200 mg, 10% on carbon(wet)). After stirring at room temperature for 2 h under H₂ atmosphere (˜0.1 MPa), the reaction was filtered and the filtrate was concentrated to afford tert-butyl (2R,5S)-5-methyl-2-(methyl-d₃)piperazine-1-carboxylate (320 mg, 82%) as a yellow oil. LC/MS ESI (m/z): 218 (M+H)⁺.

### Step 11. 7-Bromo-4-chloro-5-(2-fluoro-4-nitrophenyl)-5H-pyrrolo[3,2-d]pyrimidine

A solution of 7-bromo-4-chloro-5H-pyrrolo[3,2-d]pyrimidine (2.3 g, 10 mmol) and 1,2-difluoro-4-nitrobenzene (9.5 g, 60 mmol) in CH₃CN (30 mL) was treated with Cs₂CO₃ (4.9 g, 15 mmol). The reaction was stirred at 100° C. under N₂ overnight. The crude product was obtained by filtration and washed with water, CH₃CN and EtOH to afford 7-bromo-4-chloro-5-(2-fluoro-4-nitrophenyl)-5H-pyrrolo[3,2-d]pyrimidine (2.4 g, 70% yield) as a light brown solid. LC/MS ESI (m/z): 371, 373 (M+H)⁺.

### Step 12. 4-(7-Bromo-4-chloro-5H-pyrrolo[3,2-d]pyrimidin-5-yl)-3-fluoroaniline

To a solution of 7-bromo-4-chloro-5-(2-fluoro-4-nitrophenyl)-5H-pyrrolo[3,2-d]pyrimidine (1.1 g, 3.0 mmol) in EtOH (20 mL) and H₂O (2 mL) were added Fe (1.7 g, 30 mmol) and NH₄Cl (2.4 g, 45 mmol). After stirring at 80° C. under N₂ overnight, the reaction filtered. The filtrate was partitioned between EtOAc and NaHCO₃ (aq.). The aqueous layer was extracted twice with EtOAc. The combined organic layers were washed with brine, dried over Na₂SO₄, filtered and concentrated to afford 4-(7-bromo-4-chloro-5H-pyrrolo[3,2-d]pyrimidin-5-yl)-3-fluoroaniline (1.0 g crude) as a yellow solid. LC/MS ESI (m/z): 341, 343 (M+H)⁺.

### Step 13. (3R,6S)-1-Benzyl-6-methyl-3-(methyl-d₃)piperazine-2,5-dione

To a solution of 4-(7-bromo-4-chloro-5H-pyrrolo[3,2-d]pyrimidin-5-yl)-3-fluoroaniline (1.0 g crude) in THE (20 mL) was added isopentyl nitrite (1.4 g, 12 mmol) dropwise. The reaction mixture was stirred at 70° C. under N2 overnight. The solvent was removed under vacuum and the residue was purified by flash column chromatography (silica gel, 0˜10%, ethyl acetate in petroleum ether) to afford 7-bromo-4-chloro-5-(2-fluorophenyl)-5H-pyrrolo[3,2-d]pyrimidine (660 mg, 67% yield) as a white solid. LC/MS ESI (m/z): 326, 328 (M+H)⁺.

### Step 14. 7-Bromo-4-fluoro-5-(2-fluorophenyl)-5H-pyrrolo[3,2-d]pyrimidine

A mixture of TBAF (5.0 mL, 1.0 M in THF) and 7-bromo-4-chloro-5-(2-fluorophenyl)-5H-pyrrolo[3,2-d]pyrimidine (200 mg, 0.61 mmol) was stirred at 0° C. for 2 h. The reaction was then partitioned between DCM and ice water. The aqueous layer was extracted twice with DCM and the combined organic layers were dried over Na₂SO₄, filtered and concentrated. The residue was purified by flash column chromatography (silica gel, 0˜30%, ethyl acetate in petroleum ether) to afford 7-bromo-4-fluoro-5-(2-fluorophenyl)-5H-pyrrolo[3,2-d]pyrimidine (170 mg, 86%) as a white solid. LC/MS ESI (m/z): 310, 312 (M+H)⁺.

### Step 15. tert-Butyl (2R,5S)-4-(7-bromo-5-(2-fluorophenyl)-5H-pyrrolo[3,2-d]pyrimidin-4-yl)-5-methyl-2-(methyl-d₃)piperazine-1-carboxylate

To a solution of 7-bromo-4-fluoro-5-(2-fluorophenyl)-5H-pyrrolo[3,2-d]pyrimidine (86 mg, 0.27 mmol) in DIPEA (2 mL) was added tert-butyl (2R,5S)-5-methyl-2-(methyl-d₃)piperazine-1-carboxylate (60 mg, 0.27 mmol). The resulting mixture was stirred at 150° C. for 2 h. After cooling to room temperature, the reaction was concentrated and the residue was purified by flash column chromatography (silica gel, 0˜40%, ethyl acetate in petroleum ether) to afford tert-butyl (2R,5S)-4-(7-bromo-5-(2-fluorophenyl)-5H-pyrrolo[3,2-d]pyrimidin-4-yl)-5-methyl-2-(methyl-d₃)piperazine-1-carboxylate (80 mg, 57%) as a yellow oil. LC/MS ESI (m/z): 507, 509 (M+H)⁺.

### Step 16. (4-((2S,5R)-4-(tert-Butoxycarbonyl)-2-methyl-5-(methyl-d₃)piperazin-1-yl)-5-(2-fluorophenyl)-5H-pyrrolo[3,2-d]pyrimidin-7-yl)boronic acid

To a solution of tert-butyl (2R,5S)-4-(7-bromo-5-(2-fluorophenyl)-5H-pyrrolo[3,2-d]pyrimidin-4-yl)-5-methyl-2-(methyl-d₃)piperazine-1-carboxylate (40 mg, 0.08 mmol) and (i-PrO)₃B (89 mg, 0.47 mmol) in THE (2 mL) at −78° C. was added n-BuLi (0.11 mL, 2.5 M in hexane). After stirring at −78° C. for 1 h, the reaction was quenched with water and extracted twice with EtOAc. The combined organic layers were washed with brine, dried over Na₂SO₄, filtered and concentrated. The residue was used directly in next steps. LC/MS ESI (m/z): 473 (M+H)⁺.

### Step 17. tert-Butyl (2R,5S)-4-(7-(4-cyanopyridin-2-yl)-5-(2-fluorophenyl)-5H-pyrrolo[3,2-d]pyrimidin-4-yl)-5-methyl-2-(methyl-d₃)piperazine-1-carboxylate

To a solution of (4-((2S,5R)-4-(tert-butoxycarbonyl)-2-methyl-5-(methyl-d₃)piperazin-1-yl)-5-(2-fluorophenyl)-5H-pyrrolo[3,2-d]pyrimidin-7-yl)boronic acid (from step 16 above) in dioxane (5 mL) and H₂O (1 mL) were added 2-bromoisonicotinonitrile (23 mg, 0.12 mmol), K₂CO₃ (35 mg, 0.25 mmol) and Pd(dppf)Cl₂ (6.2 mg, 0.01 mmol). The resulting mixture was stirred at 90° C. overnight. After cooling to room temperature, the reaction was concentrated and the residue was purified by flash column chromatography (silica gel, 0˜40%, ethyl acetate in petroleum ether) to give crude product which was further purified by prep-HPLC to afford tert-butyl (2R,5S)-4-(7-(4-cyanopyridin-2-yl)-5-(2-fluorophenyl)-5H-pyrrolo[3,2-d]pyrimidin-4-yl)-5-methyl-2-(methyl-d₃)piperazine-1-carboxylate (6.7 mg, 15%) as a white solid. LC/MS ESI (m/z): 531 (M+H)⁺. ¹H NMR (400 MHz, CDCl₃) δ 9.08 (s, 1H), 8.68 (s, 1H), 8.65 (d, J=4.9 Hz, 1H), 8.20 (s, 1H), 7.47-7.22 (m, 5H), 4.10 (br. s, 1H), 3.79 (br. s, 1H), 3.34-2.96 (m, 3H), 2.55-2.11 (m, 1H), 1.36 (s, 9H), 0.83 (d, J=6.3 Hz, 3H).

### Example 212. Synthesis of tert-butyl (2R,5S)-4-(7-(4-cyanopyridin-2-yl)-5-cyclobutyl-7H-pyrrolo[2,3-d]pyrimidin-4-yl)-2,5-dimethylpiperazine-1-carboxylate (Compound 710)

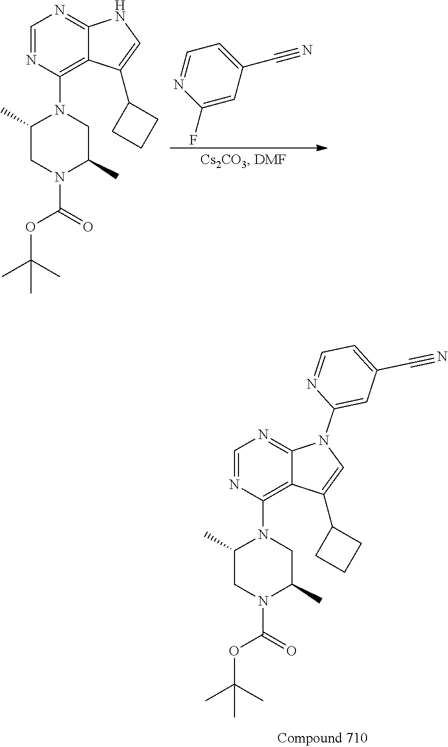

To a solution of tert-butyl (2R,5S)-4-(5-cyclobutyl-7H-pyrrolo[2,3-d]pyrimidin-4-yl)-2,5-dimethylpiperazine-1-carboxylate (500 mg, 1.3 mmol, prepared following a similar procedure outlined for compound 641) in DMF (5.0 mL) were added 2-fluoropyridine-4-carbonitrile (320 mg, 2.6 mmol) and Cs₂CO₃ (430 mg, 1.3 mmol). The mixture was stirred at 50° C. under N₂ overnight. After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by column chromatography (silica gel, 0˜30%, ethyl acetate in petroleum ether) and prep-HPLC to afford tert-butyl (2R,5S)-4-(7-(4-cyanopyridin-2-yl)-5-cyclobutyl-7H-pyrrolo[2,3-d]pyrimidin-4-yl)-2,5-dimethylpiperazine-1-carboxylate (40 mg, 6.0%) as a white solid. LC/MS ESI (m/z): 488 (M+H)⁺. ¹H NMR (400 MHz, DMSO-d6) δ 9.15 (s, 1H), 8.78 (dd, J=5.0, 0.7 Hz, 1H), 8.50 (s, 1H), 8.08 (s, 1H), 7.80 (dd, J=5.0, 1.4 Hz, 1H), 4.45 (br. s, 1H), 4.25 (br. s, 1H), 3.77-3.63 (m, 3H), 3.58 (m, 1H), 3.43 (d, J=12.6 Hz, 1H), 2.47-2.34 (m, 2H), 2.28-2.13 (m, 1H), 2.11-1.96 (m, 2H), 1.93-1.77 (m, 1H), 1.44 (s, 9H), 1.06 (d, J=6.6 Hz, 3H), 1.00 (d, J=6.8 Hz, 3H).

### Example 213. Synthesis of tert-butyl (2R,5S)-4-(7-(4-cyanopyridin-2-yl)-5-cyclopropyl-7H-pyrrolo[2,3-d]pyrimidin-4-yl)-5-methyl-2-(methyl-d₃)piperazine-1-carboxylate Compound 711)

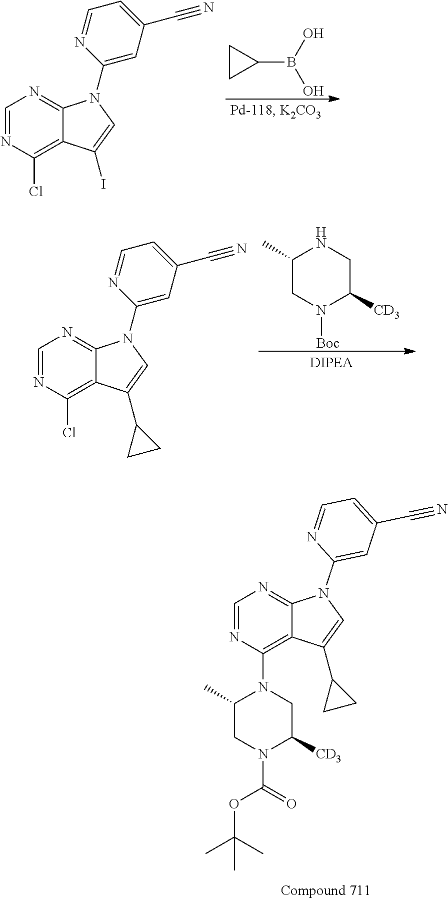

### Step 1. 2-(4-Chloro-5-cyclopropyl-7H-pyrrolo[2,3-d]pyrimidin-7-yl)isonicotinonitrile

To a solution of 2-(4-chloro-5-iodo-7H-pyrrolo[2,3-d]pyrimidin-7-yl)isonicotinonitrile (500 mg, 1.3 mmol, prepared following the procedure outlined for compound 707) in toluene (15 mL) were added cyclopropylboronic acid (230 mg, 2.5 mmol), Pd-118 (86 mg, 0.13 mmol) and K₂CO₃ (3.6 g, 26 mmol). The resulting mixture was stirred at 80° C. overnight. After cooling to room temperature, the reaction was concentrated and the residue was purified by flash column chromatography (silica gel, 0˜20%, ethyl acetate in petroleum ether) to afford 2-(4-chloro-5-cyclopropyl-7H-pyrrolo[2,3-d]pyrimidin-7-yl)isonicotinonitrile (130 mg, 34%) as a yellow solid. LC/MS ESI (m/z): 296 (M+H)⁺.

### Step 2. tert-Butyl (2R,5S)-4-(7-(4-cyanopyridin-2-yl)-5-cyclopropyl-7H-pyrrolo[2,3-d]pyrimidin-4-yl)-5-methyl-2-(methyl-d₃)piperazine-1-carboxylate

To a solution of 2-(4-chloro-5-cyclopropyl-7H-pyrrolo[2,3-d]pyrimidin-7-yl)isonicotinonitrile (61 mg, 0.20 mmol) in DIPEA (3 mL) was added tert-butyl (2R,5S)-5-methyl-2-(methyl-d₃)piperazine-1-carboxylate (30 mg, 0.13 mmol, prepared following the procedure outlined for compound 709). The resulting mixture was stirred at 150° C. for 2 h. After cooling to room temperature, the reaction was concentrated and the residue was purified by flash column chromatography (silica gel, 0˜30%, ethyl acetate in petroleum ether) to afford tert-butyl (2R,5S)-4-(7-(4-cyanopyridin-2-yl)-5-cyclopropyl-7H-pyrrolo[2,3-d]pyrimidin-4-yl)-5-methyl-2-(methyl-d₃)piperazine-1-carboxylate (25 mg, 38%) as a yellow solid. 10 mg was purified by prep-HPLC to afford 2.4 mg of the title compound as a white solid. LC/MS ESI (m/z): 477 (M+H)⁺. ¹H NMR (400 MHz, CDCl₃) δ 9.22 (s, 1H), 8.55 (d, J=4.6 Hz, 1H), 8.46 (s, 1H), 8.36 (s, 1H), 7.35 (dd, J=5.0, 1.2 Hz, 1H), 4.57-4.33 (m, 2H), 3.86-3.78 (m, 1H), 3.69 (dd, J=13.4, 4.0 Hz, 2H), 3.41 (d, J=13.6 Hz, 1H), 2.01-1.86 (m, 1H), 1.43 (s, 9H), 1.20-1.16 (m, 2H), 1.02 (d, J=6.7 Hz, 3H), 0.83-0.76 (m, 2H).

The following compound was prepared by a procedure analogous to the synthesis of compound 711 from the corresponding boronic acid.

| Cmpd | | |
|---|---|---|
| No. | Chemical Name | LCMS and ¹H NMR |
| 712 | tert-butyl (2R,5S)-4-(7-(4- | LC/MS ESI (m/z): 531 (M + H)⁺. ¹H NMR (400 MHz, |
| | cyanopyridin-2-yl)-5-(2- | CDCl₃) δ 9.35 (s, 1H), 8.55 (d, J = 5.0 Hz, 1H), 8.50 (s, |
| | fluorophenyl)-7H-pyrrolo[2,3- | 1H), 8.22 (s, 1H), 7.44-7.39 (m, 1H), 7.35-7.27 (m, 2H), |
| | d]pyrimidin-4-yl)-5-methyl-2- | 7.15 (m, 2H), 4.18-3.99 (m, 2H), 3.33-3.21 (m, 2H), |
| | (methyl-d₃)piperazine-1- | 3.20-3.11 (m, 1H), 2.87-2.68 (m, 1H), 1.36 (s, 9H), 0.90 |
| | carboxylate | (d, J = 6.6 Hz, 3H). |

### Example 214. Synthesis of tert-butyl (2R,5S)-4-(7-((S)-3,3-difluorocyclohexyl)-5-(trifluoromethyl)-7H-pyrrolo[2,3-d]pyrimidin-4-yl)-2,5-dimethylpiperazine-1-carboxylate (Compound 713) and tert-butyl (2R,5S)-4-(7-((R)-3,3-difluorocyclohexyl)-5-(trifluoromethyl)-7H-pyrrolo[2,3-d]pyrimidin-4-yl)-2,5-dimethylpiperazine-1-carboxylate (Compound 714)

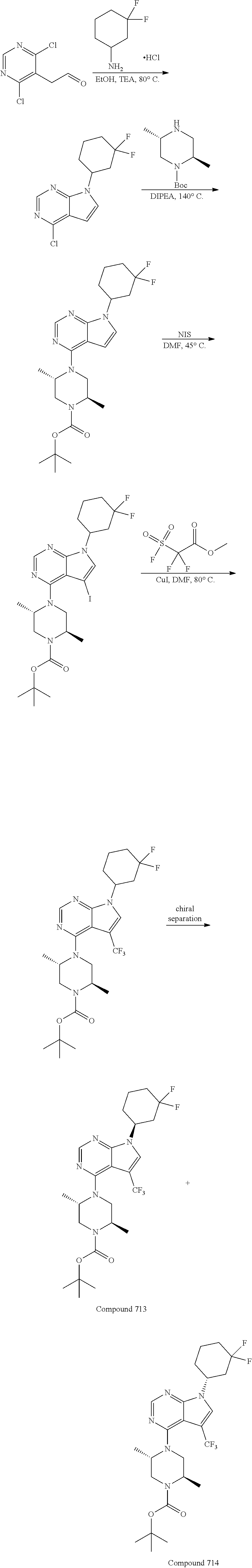

### Step 1. 4-Chloro-7-(3,3-difluorocyclohexyl)-7H-pyrrolo[2,3-d]pyrimidine

To a solution of 2-(4,6-dichloropyrimidin-5-yl)acetaldehyde (1000 mg, 5.2 mmol) in EtOH (15 mL) were added 3,3-difluorocyclohexan-1-amine HCl salt (900 mg, 5.2 mmol) and TEA (2.1 g, 21 mmol). The resulting mixture was heated to 80° C. overnight under N₂. After cooling to room temperature, the solvent was removed and the residue was purified by flash column chromatography (silica gel, 0-20%, ethyl acetate in petroleum ether) to afford 4-chloro-7-(3,3-difluorocyclohexyl)-7H-pyrrolo[2,3-d]pyrimidine (650 mg, 46%) as a solid. LC/MS ESI (m/z): 272 (M+H)⁺.

### Step 2. tert-Butyl (2R,5S)-4-(7-(3,3-difluorocyclohexyl)-7H-pyrrolo[2,3-d]pyrimidin-4-yl)-2,5-dimethylpiperazine-1-carboxylate

To a solution of 4-chloro-7-(3,3-difluorocyclohexyl)-7H-pyrrolo[2,3-d]pyrimidine (450 mg, 1.7 mmol) in DIPEA (2 mL) was added tert-butyl (2R,5S)-2,5-dimethylpiperazine-1-carboxylate (660 mg, 3.3 mmol). The resulting mixture was heated at 140° C. for 1.5 h. After cooling to room temperature, the solvent was removed and the residue was purified by flash column chromatography (silica gel, 0˜25%, ethyl acetate in petroleum ether) to afford tert-butyl (2R,5S)-4-(7-(3,3-difluorocyclohexyl)-7H-pyrrolo[2,3-d]pyrimidin-4-yl)-2,5-dimethylpiperazine-1-carboxylate (700 mg, 94%) as a solid. LC/MS ESI (m/z): 450 (M+H)⁺.

### Step 3. tert-Butyl (2R,5S)-4-(7-(3,3-difluorocyclohexyl)-5-iodo-7H-pyrrolo[2,3-d]pyrimidin-4-yl)-2,5-dimethylpiperazine-1-carboxylate

To a solution of tert-butyl (2R,5S)-4-(7-(3,3-difluorocyclohexyl)-7H-pyrrolo[2,3-d]pyrimidin-4-yl)-2,5-dimethylpiperazine-1-carboxylate (500 mg, 1.1 mmol) in DMF (10 mL) was added NIS (500 mg, 2.2 mmol) in portions. The resulting mixture was heated at 45° C. for 2 h. After cooling to room temperature, the reaction was partitioned between EtOAc and water. The aqueous layer was extracted twice with EtOAc. The combined organic layers were washed with sat. sodium thiosulfate (aq.), dried over Na₂SO₄, filtered and concentrated. The residue was purified by flash column chromatography (silica gel, 0˜20%, ethyl acetate in petroleum ether) to afford tert-butyl (2R,5S)-4-(7-(3,3-difluorocyclohexyl)-5-iodo-7H-pyrrolo[2,3-d]pyrimidin-4-yl)-2,5-dimethylpiperazine-1-carboxylate (580 mg, 90%) as a solid. LC/MS ESI (m/z): 576 (M+H)⁺.

### Step 4. tert-Butyl (2R,5S)-4-(7-(3,3-difluorocyclohexyl)-5-(trifluoromethyl)-7H-pyrrolo[2,3-d]pyrimidin-4-yl)-2,5-dimethylpiperazine-1-carboxylate

To a solution of tert-butyl (2R,5S)-4-(7-(3,3-difluorocyclohexyl)-5-iodo-7H-pyrrolo[2,3-d]pyrimidin-4-yl)-2,5-dimethylpiperazine-1-carboxylate (580 mg, 1.0 mmol) in DMF (20 mL) were added methyl 2,2-difluoro-2-(fluorosulfonyl)acetate (580 mg, 3.0 mmol) and CuI (190 mg, 1.0 mmol). The resulting mixture was heated at 80° C. overnight. After cooling to room temperature, the reaction was filtered and partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0˜20%, ethyl acetate in petroleum ether) to afford tert-butyl (2R,5S)-4-(7-(3,3-difluorocyclohexyl)-5-(trifluoromethyl)-7H-pyrrolo[2,3-d]pyrimidin-4-yl)-2,5-dimethylpiperazine-1-carboxylate (360 mg, 69%) as a solid. LC/MS ESI (m/z): 518 (M+H)⁺.

### Step 5. tert-Butyl (2R,5S)-4-(7-((S)-3,3-difluorocyclohexyl)-5-(trifluoromethyl)-7H-pyrrolo[2,3-d]pyrimidin-4-yl)-2,5-dimethylpiperazine-1-carboxylate and tert-butyl (2R,5S)-4-(7-((R)-3,3-difluorocyclohexyl)-5-(trifluoromethyl)-7H-pyrrolo[2,3-d]pyrimidin-4-yl)-2,5-dimethylpiperazine-1-carboxylate

tert-Butyl (2R,5S)-4-(7-(3,3-difluorocyclohexyl)-5-(trifluoromethyl)-7H-pyrrolo[2,3-d]pyrimidin-4-yl)-2,5-dimethylpiperazine-1-carboxylate (220 mg) was separated by chiral HPLC (column: CHIRALPAK AD-H (ADH0CE-XG136), 0.46 cm I.D.×25 cm L., mobile phase: hexane/IPA=90/10 (V/V)) to afford two isomers.

Peak 1: retention time: 4.7 min, tert-butyl (2R,5S)-4-(7-((S)-3,3-difluorocyclohexyl)-5-(trifluoromethyl)-7H-pyrrolo[2,3-d]pyrimidin-4-yl)-2,5-dimethylpiperazine-1-carboxylate (assigned as compound 713 without additional confirmation, 95 mg) as a white solid. LC/MS ESI (m/z): 518 (M+H)⁺. ¹H NMR (400 MHz, CDCl₃) δ 8.39 (s, 1H), 7.41 (s, 1H), 4.92-4.80 (m, 1H), 4.53-4.16 (m, 2H), 3.69-3.59 (m, 2H), 3.58-3.50 (m, 1H), 3.42-3.34 (m, 1H), 2.55-2.44 (m, 1H), 2.30-2.08 (m, 3H), 1.95-1.67 (m, 4H), 1.42 (s, 9H), 1.08-1.01 (m, 6H)

Peak 2: retention time: 5.502 min, tert-butyl (2R,5S)-4-(7-((R)-3,3-difluorocyclohexyl)-5-(trifluoromethyl)-7H-pyrrolo[2,3-d]pyrimidin-4-yl)-2,5-dimethylpiperazine-1-carboxylate (assigned as compound 714 without additional confirmation, 86 mg) as a white solid. LC/MS ESI (m/z): 518 (M+H)⁺. ¹H NMR (400 MHz, CDCl₃) δ 8.39 (s, 1H), 7.41 (s, 1H), 4.94-4.83 (m, 1H), 4.53-4.17 (m, 2H), 3.69-3.59 (m, 2H), 3.57-3.49 (m, 1H), 3.38 (d, J=13.4 Hz, 1H), 2.59-2.47 (m, 1H), 2.32-2.06 (m, 3H), 1.95-1.87 (m, 1H), 1.83-1.68 (m, 3H), 1.42 (s, 9H), 1.07-1.01 (m, 6H).

### Example 215. Synthesis of tert-butyl (2R,5S)-4-(5-cyclopropyl-7-(4-ethynylpyridin-2-yl)-7H-pyrrolo[2,3-d]pyrimidin-4-yl)-2,5-dimethylpiperazine-1-carboxylate (Compound 715)

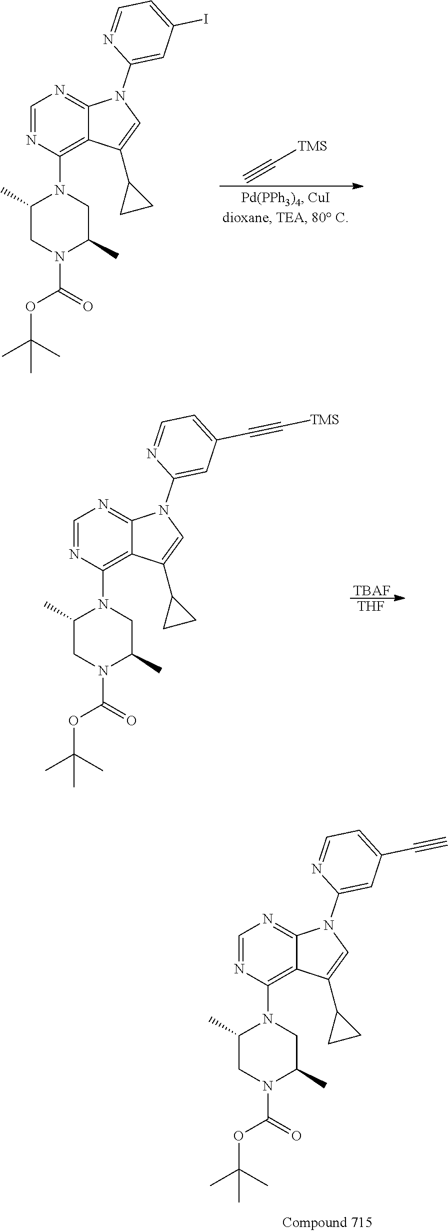

### Step 1. tert-Butyl (2R,5S)-4-(5-cyclopropyl-7-(4-((trimethylsilyl)ethynyl)pyridin-2-yl)-7H-pyrrolo[2,3-d]pyrimidin-4-yl)-2,5-dimethylpiperazine-1-carboxylate

To a solution of tert-butyl (2R,5S)-4-(5-cyclopropyl-7-(4-iodopyridin-2-yl)-7H-pyrrolo[2,3-d]pyrimidin-4-yl)-2,5-dimethylpiperazine-1-carboxylate (100 mg, 0.17 mmol, prepared following the procedure outlined for compound 717) in dioxane (6 m1) were added ethynyltrimethylsilane (83 mg, 0.85 mmol), Pd(PPh₃)₄ (23 mg, 0.02 mmol), CuI (16 mg, 0.09 mmol) and TEA (170 mg, 1.7 mmol). The reaction was stirred at 80° C. under N₂ overnight. After cooling to room temperature, solvent was removed and the residue was purified by flash column chromatography (silica gel, 0-30% ethyl acetate in petroleum ether) to afford tert-butyl (2R,5S)-4-(5-cyclopropyl-7-(4-((trimethylsilyl)ethynyl)pyridin-2-yl)-7H-pyrrolo[2,3-d]pyrimidin-4-yl)-2,5-dimethylpiperazine-1-carboxylate (80 mg, 86%) as a solid. LC/MS ESI (m/z): 545 (M+H)⁺.

### Step 2. tert-Butyl (2R,5S)-4-(5-cyclopropyl-7-(4-ethynylpyridin-2-yl)-7H-pyrrolo[2,3-d]pyrimidin-4-yl)-2,5-dimethylpiperazine-1-carboxylate

To a solution of tert-butyl (2R,5S)-4-(5-cyclopropyl-7-(4-((trimethylsilyl)ethynyl)pyridin-2-yl)-7H-pyrrolo[2,3-d]pyrimidin-4-yl)-2,5-dimethylpiperazine-1-carboxylate (80 mg, 0.15 mmol) in THE (1 mL) was added TBAF (1.0 mL, 1.0 M in THF). After 1 h, the reaction was diluted with EtOAc and washed with sat. NH₄Cl (aq.). The aqueous layer was extracted with EtOAc. The organic layer was washed with sat. NH₄Cl (aq.), dried over Na₂SO₄, filtered and concentrated. The residue was purified by prep-HPLC to afford tert-butyl (2R,5S)-4-(5-cyclopropyl-7-(4-ethynylpyridin-2-yl)-7H-pyrrolo[2,3-d]pyrimidin-4-yl)-2,5-dimethylpiperazine-1-carboxylate (35 mg, 49%) as a white solid. LC/MS ESI (m/z): 473 (M+H)⁺. ¹H NMR (400 MHz, CD₃OD) δ 8.80 (s, 1H), 8.44 (d, J=4.0 Hz, 1H), 8.40 (s, 1H), 7.80 (d, J=0.9 Hz, 1H), 7.29 (dd, J=5.1, 1.3 Hz, 1H), 4.89 (m, 1H), 4.40 (br. s, 1H), 3.98 (s, 1H), 3.83-3.72 (m, 3H), 3.64 (d, J=12.3 Hz, 1H), 2.09-2.02 (m, 1H), 1.50 (s, 9H), 1.16 (m, 6H), 1.09-1.03 (m, 2H), 0.91 (m, 1H), 0.70-0.64 (m, 1H).

The following compound was prepared by a procedure similar to the synthesis of compound 715 from the corresponding aryl iodide (tert-butyl (2R,5S)-4-(7-(4-iodopyridin-2-yl)-5-(trifluoromethyl)-7H-pyrrolo[2,3-d]pyrimidin-4-yl)-2,5-dimethylpiperazine-1-carboxylate), which was prepared following similar procedures as outlined for compound 717.

| Cmpd | | |
|---|---|---|
| No. | Chemical Name | LCMS and ¹H NMR |
| 716 | tert-butyl (2R,5S)-4-(7-(4- | LC/MS ESI (m/z): 501 (M + H)⁺. ¹H NMR (400 MHz, |
| | ethynylpyridin-2-yl)-5- | CDCl₃) δ 8.92 (s, 1H), 8.61 (s, 1H), 8.57 (s, 1H), 8.48 (dd, |
| | (trifluoromethyl)-7H- | J = 5.0, 0.6 Hz, 1H), 7.32 (dd, J = 5.0, 1.3 Hz, 1H), 4.62- |
| | pyrrolo[2,3-d]pyrimidin-4-yl)- | 4.33 (m, 2H), 3.80-3.70 (m, 2H), 3.67-3.61 (m, 1H), |
| | 2,5-dimethylpiperazine-1- | 3.47 (d, J = 12 Hz, 1H), 3.40 (s, 1H), 1.49 (s, 9H), 1.16- |
| | carboxylate | 1.10 (m, 6H). |

### Example 216. Synthesis of tert-butyl (2R,5S)-4-(5-cyclopropyl-7-(4-ethylpyridin-2-yl)-7H-pyrrolo[2,3-d]pyrimidin-4-yl)-2,5-dimethylpiperazine-1-carboxylate (Compound 717)

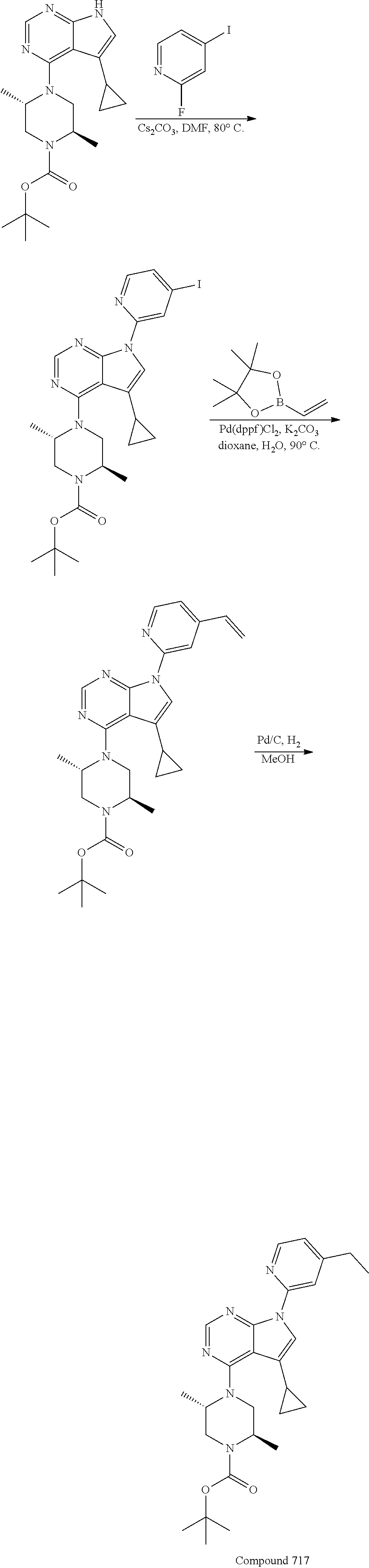

### Step 1. tert-Butyl (2R,5S)-4-(5-cyclopropyl-7-(4-iodopyridin-2-yl)-7H-pyrrolo[2,3-d]pyrimidin-4-yl)-2,5-dimethylpiperazine-1-carboxylate

To a solution of tert-butyl (2R,5S)-4-(5-cyclopropyl-7H-pyrrolo[2,3-d]pyrimidin-4-yl)-2,5-dimethylpiperazine-1-carboxylate (350 mg, 0.94 mmol, prepared following the procedure outlined for compound 654) in DMF (8 mL) were added 2-fluoro-4-iodopyridine (320 mg, 1.4 mmol) and Cs₂CO₃ (620 mg, 1.9 mmol). The resulting mixture was heated at 80° C. overnight. After cooling to room temperature, the reaction was diluted with water and extracted twice with EtOAc. The combined organic layers were washed with brine, dried over Na₂SO₄, filtered and concentrated. The residue was purified by flash column chromatography (silica gel, 0˜30% ethyl acetate in petroleum ether) to afford tert-butyl (2R,5S)-4-(5-cyclopropyl-7-(4-iodopyridin-2-yl)-7H-pyrrolo[2,3-d]pyrimidin-4-yl)-2,5-dimethylpiperazine-1-carboxylate (400 mg, 74%) as a white solid. LC/MS ESI (m/z): 575 (M+H)⁺.

### Step 2. tert-Butyl (2R,5S)-4-(5-cyclopropyl-7-(4-vinylpyridin-2-yl)-7H-pyrrolo[2,3-d]pyrimidin-4-yl)-2,5-dimethylpiperazine-1-carboxylate

To a solution of tert-butyl (2R,5S)-4-(5-cyclopropyl-7-(4-iodopyridin-2-yl)-7H-pyrrolo[2,3-d]pyrimidin-4-yl)-2,5-dimethylpiperazine-1-carboxylate (100 mg, 0.17 mmol) in dioxane (6 m1) and water (2 drops) were added 4,4,5,5-tetramethyl-2-vinyl-1,3,2-dioxaborolane (31 mg, 0.20 mmol), Pd(dppf)Cl₂ (16 mg, 0.02 mmol) and K₂CO₃ (70 mg, 0.51 mmol). The reaction was stirred at 90° C. under N₂ overnight. After cooling to room temperature, the solvent was removed and the residue was purified by flash column chromatography (silica gel, 0˜30% ethyl acetate in petroleum ether) to afford tert-butyl (2R,5S)-4-(5-cyclopropyl-7-(4-vinylpyridin-2-yl)-7H-pyrrolo[2,3-d]pyrimidin-4-yl)-2,5-dimethylpiperazine-1-carboxylate (40 mg, 50%) as a white solid. LC/MS ESI (m/z): 475 (M+H)⁺.

### Step 3. tert-Butyl (2R,5S)-4-(5-cyclopropyl-7-(4-ethylpyridin-2-yl)-7H-pyrrolo[2,3-d]pyrimidin-4-yl)-2,5-dimethylpiperazine-1-carboxylate

To a solution of tert-butyl (2R,5S)-4-(5-cyclopropyl-7-(4-vinylpyridin-2-yl)-7H-pyrrolo[2,3-d]pyrimidin-4-yl)-2,5-dimethylpiperazine-1-carboxylate (40 mg, 0.084 mmol) in MeOH (10 mL) was added palladium (100 mg, 10% on activated carbon (wetted with ca. 55% water)). The resulting mixture was stirred at room temperature under H₂ atmosphere (˜0.1 MPa) overnight. The reaction was filtered, and the filtrate was concentrated. The residue was purified by flash column chromatography (silica gel, 0˜30% ethyl acetate in petroleum ether) to give crude product which was further purified by prep-HPLC to afford tert-butyl (2R,5S)-4-(5-cyclopropyl-7-(4-ethylpyridin-2-yl)-7H-pyrrolo[2,3-d]pyrimidin-4-yl)-2,5-dimethylpiperazine-1-carboxylate (33 mg, 83%) as a white solid. LC/MS ESI (m/z): 477 (M+H)⁺. ¹H NMR (400 MHz, CD₃OD) δ 8.41 (s, 1H), 8.38-8.30 (m, 2H), 7.72 (s, 1H), 7.17 (m, 1H), 4.89 (m, 1H), 4.40 (br. s, 1H), 3.84-3.62 (m, 4H), 2.78 (q, J=7.6 Hz, 2H), 2.10-2.02 (m, 1H), 1.50 (s, 9H), 1.31 (t, J=4.0 Hz, 3H), 1.16 (m, 6H), 1.08-1.02 (m, 2H), 0.91 (s, 1H), 0.71-0.64 (m, 1H).

The following compound was prepared by a procedure analogous to the synthesis of compound 717 from tert-butyl (2R,5S)-2,5-dimethyl-4-(5-(trifluoromethyl)-7H-pyrrolo[2,3-d]pyrimidin-4-yl)piperazine-1-carboxylate which was prepared following the procedures outlined for compound 602.

| Cmpd | | |
|---|---|---|
| No. | Chemical Name | LCMS and ¹H NMR |
| 718 | tert-butyl (2R,5S)-4-(7-(4- | LC/MS ESI (m/z): 505 (M + H)⁺. ¹H NMR (400 MHz, |
| | ethylpyridin-2-yl)-5- | CDCl₃) δ 8.61-8.53 (m, 3H), 8.40 (d, J = 5.1 Hz, 1H), |
| | (trifluoromethyl)-7H- | 7.14 (d, J = 4.6 Hz, 1H), 4.59-4.31 (m, 2H), 3.80-3.70 |
| | pyrrolo[2,3-d]pyrimidin-4-yl)- | (m, 2H), 3.68-3.62 (m, 1H), 3.47 (d, J = 12 Hz, 1H), 2.82 |
| | 2,5-dimethylpiperazine-1- | (q, J = 7.6 Hz, 2H), 1.49 (s, 9H), 1.35 (t, J = 7.6 Hz, 3H), |
| | carboxylate | 1.17-1.10 (m, 6H). |

### Example 217. Fluorescent TRPML Assays

### TRPML1 Assay

### Cell Culture

HEK-293 Trex cells were stably transfected with a construct consisting of the human coding sequence for TRPML1 cloned into the tert-inducible plasmid pCDNA5 T/O. Mutations were introduced into the TRPML1 sequence to facilitate expression on the cell surface (Silvia Vergarajauregui, Rosa Puertollano, Traffic. 2006 March; 7(3): 337-353). Briefly, the cells are cultured in 150 mm round tissue culture dishes containing 20 mL of media. The day before the assay the cells are rinsed with DPBS-Ca—Mg and then treated briefly with Trypsin-EDTA. The Trypsin-EDTA is diluted with growth media, and cells are counted. 38×10\hat{ }6 cells are re-plated into 150 mm round tissue culture dishes in media containing 0.5 ug/mL doxycycline to induce expression of hTRPML1.

### Dye Loading

The day of the experiment cells are lifted from the plates as above and collected by centrifugation. The cells are then suspended in dye loading buffer consisting of Ringer's solution supplemented with 0.1% Pluronic Acid and 1 micromolar Fluo4-AM dye. Cells are loaded for ˜60 minutes in the dark with occasional mixing. The cells are collected by centrifugation, the loading media aspirated, and the cells resuspended in 25 mL Ringer's solution and incubated ˜60 minutes in the dark. The cells are again collected by centrifugation, rinsed in Ringer's Solution and resuspended to 0.2×10≢cells/mL in modified Ringer's solution containing 10 mM calcium.

### Compound Assay Plates

Compounds are dissolved to a concentration of 10 millimolar with DMSO. Compound plates are created by dispensing compounds into 384 well black wall clear bottom plates (Greiner 781091). Positive and negative controls are included on each plate. Typically, different amounts of each compound are tested ranging from 100 nanoMoles (20 micromolar final concentration) decreasing in half-log steps to 31 picoMoles (6 nanomolar final concentration). Each concentration is typically tested in triplicate.

### Assay

50 microliters of dye-loaded cells are dispensed into each well of the compound assay plate created above. The fluorescence in each well is then determined with an excitation wavelength of 480 nM and an emission wavelength of 540 nM using either a Molecular Devices SpectraMax multimode plate reader or a Hamamatsu FDSS/uCell plate imager.

### Analysis and Statistics

The resulting fluorescence for each well is exported as an ascii file and loaded into our LIMS for analysis. The percent activity of each compound at each concentration is determined by comparison to the positive and negative control wells included in each plate.

TRPML2 and TRPML3 Assays Assays for TRPML2 and TRPML3 were performed as above for TRPML1, by substituting the appropriate TRPML2 or TRPML3 subtype for the TRPML1.

EC₅₀ values were calculated using a non-linear regression of Prism. The EC₅₀ determined for each compound using the assay is summarized in Table 3 below. The compound numbers correspond to those shown in Table 1. In the table, “A” indicates an EC₅₀ of less than 100 nM, “B” indicates an EC₅₀ range from 100 nM to 500 nM; “C” indicates an EC₅₀ range from 500 nM to 2 μM; and “D” indicates an EC₅₀ greater than 2 μM.

TABLE 3
Efficacy of exemplary compounds of the invention

| | Human | Human | Human |
|---|---|---|---|
| | TRPML1 | TRPML2 | TRPML3 |
| Cmpd No. | EC₅₀ | EC₅₀ | EC₅₀ |
| 101 | B | B | A |
| 102 | A | B | A |
| 103 | B | C | B |
| 104 | C | D | B |
| 105 | C | D | C |
| 106 | B | C | B |
| 107 | A | B | A |
| 108 | A | B | A |
| 109 | A | A | A |
| 110 | A | A | A |
| 111 | A | B | A |
| 112 | B | B | C |
| 113 | A | C | B |
| 114 | A | A | A |
| 115 | B | C | B |
| 116 | B | C | B |
| 117 | A | B | A |
| 118 | B | D | C |
| 119 | A | B | A |
| 120 | B | B | A |
| 121 | A | B | A |
| 122 | A | D | A |
| 123 | A | B | A |
| 124 | A | C | B |
| 125 | C | — | C |
| 126 | C | — | C |
| 127 | A | B | A |
| 128 | A | B | A |
| 129 | A | B | A |
| 130 | A | B | A |
| 131 | A | B | A |
| 132 | A | D | B |
| 133 | B | D | C |
| 134 | A | B | A |
| 135 | A | B | A |
| 136 | A | A | A |
| 137 | B | C | B |
| 138 | C | C | C |
| 139 | A | A | A |
| 140 | A | A | A |
| 141 | A | B | A |
| 142 | A | C | A |
| 143 | B | D | C |
| 144 | A | C | A |
| 145 | B | A | A |
| 146 | B | B | A |
| 147 | B | B | A |
| 148 | A | B | A |
| 149 | B | C | B |
| 150 | B | C | B |
| 151 | B | B | B |
| 152 | C | D | C |
| 153 | D | — | D |
| 154 | A | A | A |
| 155 | B | C | B |
| 156 | B | B | A |
| 157 | B | B | A |
| 158 | A | A | A |
| 159 | B | B | B |
| 160 | B | B | A |
| 161 | B | C | B |
| 162 | C | C | B |
| 163 | C | C | B |
| 164 | A | B | A |
| 165 | B | C | B |
| 166 | B | C | B |
| 167 | B | C | A |
| 168 | C | D | C |
| 169 | A | C | A |
| 170 | B | C | A |
| 171 | C | D | D |
| 172 | C | D | C |
| 173 | C | D | C |
| 174 | B | C | B |
| 175 | C | D | C |
| 176 | B | C | B |
| 177 | C | D | C |
| 178 | B | D | C |
| 179 | C | D | B |
| 180 | B | C | A |
| 181 | B | C | B |
| 182 | C | D | C |
| 183 | B | C | B |
| 184 | B | C | B |
| 185 | A | C | A |
| 186 | A | B | A |
| 187 | B | B | B |
| 188 | B | C | B |
| 189 | B | — | B |
| 190 | C | C | B |
| 191 | B | C | B |
| 192 | A | B | A |
| 193 | A | B | A |
| 194 | B | B | B |
| 195 | B | B | A |
| 196 | A | B | A |
| 197 | B | B | B |
| 198 | A | B | A |
| 199 | B | B | B |
| 200 | A | B | A |
| 201 | B | C | B |
| 202 | B | C | B |
| 203 | A | B | A |
| 204 | A | B | A |
| 205 | B | C | B |
| 206 | A | C | B |
| 207 | A | B | A |
| 208 | A | B | A |
| 209 | A | B | A |
| 210 | A | A | A |
| 211 | B | C | A |
| 212 | A | B | A |
| 213 | B | C | B |
| 214 | B | C | B |
| 215 | B | C | B |
| 216 | A | C | B |
| 217 | B | C | B |
| 218 | A | C | A |
| 219 | A | B | A |
| 220 | A | B | A |
| 221 | A | B | A |
| 222 | A | C | A |
| 223 | B | C | B |
| 224 | A | B | A |
| 225 | A | B | A |
| 226 | B | C | B |
| 227 | C | — | B |
| 228 | B | C | B |
| 229 | C | C | B |
| 230 | B | B | A |
| 231 | A | B | A |
| 232 | A | C | A |
| 233 | C | C | B |
| 234 | B | C | B |
| 235 | B | B | B |
| 236 | A | B | A |
| 237 | C | C | B |
| 238 | A | C | B |
| 239 | A | C | A |
| 240 | B | C | B |
| 241 | B | C | B |
| 242 | B | B | B |
| 243 | B | C | B |
| 244 | C | C | B |
| 245 | B | C | B |
| 246 | C | C | B |
| 247 | C | C | B |
| 248 | C | D | C |
| 249 | D | D | C |
| 250 | C | D | B |
| 251 | D | D | C |
| 252 | B | D | B |
| 253 | A | B | A |
| 254 | B | D | B |
| 255 | B | C | B |
| 256 | A | C | A |
| 257 | B | C | B |
| 258 | A | C | A |
| 259 | B | C | B |
| 260 | A | A | A |
| 261 | C | D | C |
| 262 | A | C | B |
| 263 | B | C | B |
| 264 | B | C | B |
| 265 | A | B | A |
| 266 | A | C | A |
| 267 | B | B | A |
| 268 | A | A | A |
| 269 | B | C | A |
| 270 | C | D | B |
| 271 | B | B | B |
| 272 | B | C | B |
| 273 | B | C | B |
| 274 | B | C | B |
| 275 | D | D | C |
| 276 | A | — | — |
| 277 | A | — | — |
| 278 | C | — | — |
| 279 | B | — | — |
| 280 | B | — | — |
| 281 | A | — | — |
| 282 | B | — | — |
| 283 | D | — | B |
| 284 | C | — | — |
| 285 | A | — | — |
| 286 | B | — | — |
| 287 | A | — | — |
| 288 | B | — | — |
| 289 | B | — | — |
| 290 | A | — | — |
| 291 | A | — | — |
| 292 | B | — | — |
| 293 | C | — | — |
| 294 | C | — | — |
| 295 | C | — | — |
| 296 | B | — | — |
| 297 | C | — | — |
| 298 | C | — | — |
| 299 | C | D | C |
| 300 | A | C | A |
| 301 | B | C | B |
| 302 | C | D | B |
| 303 | B | C | B |
| 304 | C | C | B |
| 305 | C | D | B |
| 306 | C | C | B |
| 307 | C | C | A |
| 308 | A | A | A |
| 309 | D | D | C |
| 310 | A | A | A |
| 311 | A | A | A |
| 312 | B | C | A |
| 313 | D | D | C |
| 314 | C | D | C |
| 315 | C | — | C |
| 316 | C | — | A |
| 317 | C | D | B |
| 318 | A | C | B |
| 319 | C | C | B |
| 320 | A | B | — |
| 321 | C | D | C |
| 322 | B | C | A |
| 323 | B | D | A |
| 324 | A | C | A |
| 325 | B | B | A |
| 326 | B | D | A |
| 327 | B | C | A |
| 328 | C | D | B |
| 329 | C | D | B |
| 330 | A | B | A |
| 331 | A | A | A |
| 332 | A | C | A |
| 333 | A | A | A |
| 334 | A | B | A |
| 335 | A | C | A |
| 336 | B | D | A |
| 337 | C | D | B |
| 338 | B | D | C |
| 339 | B | D | B |
| 340 | C | D | B |
| 341 | A | A | A |
| 342 | A | C | B |
| 343 | C | D | B |
| 344 | B | D | B |
| 345 | C | D | C |
| 346 | A | A | A |
| 347 | A | A | A |
| 348 | A | A | A |
| 349 | A | A | A |
| 350 | A | A | A |
| 351 | A | C | — |
| 352 | A | A | A |
| 353 | A | A | A |
| 354 | A | A | A |
| 355 | A | A | A |
| 356 | A | — | — |
| 357 | A | — | — |
| 358 | A | — | — |
| 359 | B | — | — |
| 360 | C | — | — |
| 361 | A | — | — |
| 362 | A | B | A |
| 363 | B | C | A |
| 364 | A | B | A |
| 365 | A | B | A |
| 366 | A | B | A |
| 367 | A | B | A |
| 368 | A | C | A |
| 369 | B | C | B |
| 370 | A | B | A |
| 371 | A | C | A |
| 372 | B | — | B |
| 373 | B | — | A |
| 374 | B | C | A |
| 375 | A | B | A |
| 376 | A | B | A |
| 377 | A | B | A |
| 378 | A | A | A |
| 379 | C | D | C |
| 380 | B | — | C |
| 381 | D | D | D |
| 382 | C | — | C |
| 383 | B | — | D |
| 384 | A | B | A |
| 385 | B | — | B |
| 386 | A | — | B |
| 387 | C | B | A |
| 388 | A | A | A |
| 389 | C | B | A |
| 390 | A | A | A |
| 391 | A | B | A |
| 392 | B | — | A |
| 393 | A | — | A |
| 394 | A | — | A |
| 395 | A | — | A |
| 396 | B | — | A |
| 397 | D | — | B |
| 398 | A | B | B |
| 399 | B | C | A |
| 400 | C | D | C |
| 401 | B | D | A |
| 402 | A | C | A |
| 403 | B | C | A |
| 404 | A | A | A |
| 405 | A | A | A |
| 406 | B | C | C |
| 407 | A | A | A |
| 408 | B | B | A |
| 409 | A | B | A |
| 410 | C | D | C |
| 411 | A | A | A |
| 412 | B | B | A |
| 413 | B | B | A |
| 414 | C | D | C |
| 415 | A | B | A |
| 416 | A | A | A |
| 417 | A | B | A |
| 418 | A | B | A |
| 419 | A | B | A |
| 420 | A | A | A |
| 421 | B | A | A |
| 422 | A | B | A |
| 423 | A | A | A |
| 424 | A | B | A |
| 425 | A | B | A |
| 426 | A | A | A |
| 427 | A | A | A |
| 428 | A | A | A |
| 429 | A | A | A |
| 430 | A | B | A |
| 431 | B | C | A |
| 432 | B | C | A |
| 433 | B | C | B |
| 434 | A | A | A |
| 435 | A | A | A |
| 436 | A | B | A |
| 437 | A | A | A |
| 438 | A | C | A |
| 439 | B | C | A |
| 440 | D | D | C |
| 441 | B | D | B |
| 442 | A | C | A |
| 443 | A | A | A |
| 444 | A | B | A |
| 445 | A | B | A |
| 446 | A | B | A |
| 447 | A | A | A |
| 448 | A | B | A |
| 449 | A | B | A |
| 450 | A | — | B |
| 451 | B | D | B |
| 452 | A | B | A |
| 453 | A | B | A |
| 454 | A | A | A |
| 455 | A | B | A |
| 456 | C | C | B |
| 457 | A | A | A |
| 458 | A | B | B |
| 459 | A | — | A |
| 460 | A | B | A |
| 461 | A | B | A |
| 462 | A | B | A |
| 463 | A | A | A |
| 464 | C | — | B |
| 465 | B | C | A |
| 466 | A | C | A |
| 467 | D | — | C |
| 468 | B | D | B |
| 469 | B | C | B |
| 470 | C | — | C |
| 471 | C | C | B |
| 472 | B | C | B |
| 473 | B | C | B |
| 474 | D | D | D |
| 475 | C | D | B |
| 476 | A | A | A |
| 477 | A | A | A |
| 478 | A | A | A |
| 479 | A | A | A |
| 480 | A | C | A |
| 481 | A | A | A |
| 482 | A | B | A |
| 483 | A | A | A |
| 484 | A | B | A |
| 485 | A | C | C |
| 486 | A | B | A |
| 487 | A | A | A |
| 488 | A | C | A |
| 489 | A | A | A |
| 490 | A | A | A |
| 491 | A | B | A |
| 492 | A | B | A |
| 493 | A | A | A |
| 494 | A | B | A |
| 495 | A | B | A |
| 496 | A | B | A |
| 497 | A | A | A |
| 498 | A | A | A |
| 499 | A | B | A |
| 500 | B | C | B |
| 502 | A | B | A |
| 503 | A | D | B |
| 504 | A | B | A |
| 505 | A | D | A |
| 506 | A | C | A |
| 507 | A | A | A |
| 508 | A | — | A |
| 509 | A | A | A |
| 510 | B | D | A |
| 511 | A | A | A |
| 512 | A | B | A |
| 513 | A | C | A |
| 514 | A | A | A |
| 515 | A | — | A |
| 516 | A | A | A |
| 517 | A | B | A |
| 518 | A | A | A |
| 519 | A | B | A |
| 520 | A | B | A |
| 521 | A | C | A |
| 522 | B | B | A |
| 523 | A | A | A |
| 524 | A | A | A |
| 525 | A | A | A |
| 526 | B | B | B |
| 527 | C | D | C |
| 528 | B | A | A |
| 529 | C | D | B |
| 530 | A | A | A |
| 531 | B | — | B |
| 532 | A | C | B |
| 533 | A | A | A |
| 535 | A | A | A |
| 536 | A | A | A |
| 537 | A | A | A |
| 538 | A | B | A |
| 601 | A | B | A |
| 602 | A | C | B |
| 603 | A | C | A |
| 604 | A | B | A |
| 605 | A | B | A |
| 607 | B | — | C |
| 608 | B | D | A |
| 609 | A | C | A |
| 610 | A | C | A |
| 611 | B | — | B |
| 612 | A | C | B |
| 613 | A | — | A |
| 614 | A | B | A |
| 615 | A | A | A |
| 616 | A | A | A |
| 617 | A | D | B |
| 618 | A | B | A |
| 619 | A | C | A |
| 620 | A | B | A |
| 623 | A | C | A |
| 624 | B | D | B |
| 625 | B | D | D |
| 626 | A | — | C |
| 627 | B | D | B |
| 628 | C | — | C |
| 629 | C | D | B |
| 630 | C | — | D |
| 631 | C | — | D |
| 632 | C | — | D |
| 633 | C | D | C |
| 634 | C | — | C |
| 635 | A | B | B |
| 636 | A | A | A |
| 637 | A | B | A |
| 638 | A | B | A |
| 641 | A | A | A |
| 642 | B | C | B |
| 643 | B | C | B |
| 644 | B | C | B |
| 645 | B | B | B |
| 646 | C | D | B |
| 647 | B | C | C |
| 648 | C | C | B |
| 649 | A | A | A |
| 650 | A | A | A |
| 651 | A | A | A |
| 652 | A | B | A |
| 653 | A | A | A |
| 654 | B | D | A |
| 655 | A | A | B |
| 656 | A | B | A |
| 657 | A | A | A |
| 658 | A | C | B |
| 659 | A | D | B |
| 660 | B | C | B |
| 661 | B | C | B |
| 662 | A | B | A |
| 663 | B | D | B |
| 664 | A | C | A |
| 665 | A | C | A |
| 666 | A | B | B |
| 667 | A | B | A |
| 668 | B | C | B |
| 669 | A | C | A |
| 670 | B | C | A |
| 671 | A | B | A |
| 672 | B | C | B |
| 673 | B | D | B |
| 674 | D | — | D |
| 675 | A | B | B |
| 676 | A | B | A |
| 677 | A | B | A |
| 678 | B | B | B |
| 679 | A | A | A |
| 680 | A | A | A |
| 681 | A | A | A |
| 682 | A | A | A |
| 683 | A | B | A |
| 684 | A | A | A |
| 685 | A | C | A |
| 686 | A | A | A |
| 687 | A | B | A |
| 688 | A | A | A |
| 689 | A | B | B |
| 690 | A | C | A |
| 691 | C | — | C |
| 692 | A | A | A |
| 693 | A | B | A |
| 694 | B | — | A |
| 695 | A | B | A |
| 696 | A | A | A |
| 697 | A | A | A |
| 698 | A | B | A |
| 699 | A | B | A |
| 700 | A | B | A |
| 701 | A | D | B |
| 702 | A | C | C |
| 703 | A | A | A |
| 704 | A | A | A |
| 705 | A | C | A |
| 706 | B | C | B |
| 707 | A | A | A |
| 708 | C | C | C |
| 709 | A | A | A |
| 710 | A | B | A |
| 711 | A | C | B |
| 712 | A | A | A |
| 713 | C | — | B |
| 714 | B | C | B |
| 715 | A | A | A |
| 716 | B | C | B |
| 717 | A | B | A |
| 718 | B | C | B |

### EQUIVALENTS

It will be recognized that one or more features of any embodiments disclosed herein may be combined and/or rearranged within the scope of the invention to produce further embodiments that are also within the scope of the invention.

Those skilled in the art will recognize, or be able to ascertain using no more than routine experimentation, many equivalents to the specific embodiments of the invention described herein. Such equivalents are intended to be within the scope of the present invention.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

1. A compound of formula (Ia) 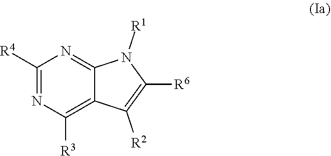
or a pharmaceutically acceptable salt thereof, wherein
R¹ is aryl, heteroaryl, cycloalkyl, or heterocycloalkyl, each R¹ optionally substituted by 1-5 independently selected R⁷;
R² is C₁₋₆ alkyl, optionally substituted by 1-5 independently selected R⁸;
R³ is 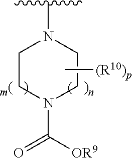
each of R⁴ and R⁶ is independently selected from the group consisting of H, hydroxy, halogen, cyano, C₁₋₆ alkyl, C₁₋₆ haloalkyl, C₁₋₆ alkoxy, C₁₋₆ haloalkoxy, C₁₋₆ alkylthio, and NR^{a}R^{b};
each of R⁷ and R⁸ are independently selected at each occurrence from the group consisting of deuterium, hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl; or
when R¹ or R² is cycloalkyl or heterocycloalkyl, two R⁷ or two R⁸ on the same carbon can be taken together to form oxo, or any two R⁷ or two R⁸ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
R⁹ is C₁₋₆ alkyl, optionally substituted with substituents independently selected from the group consisting of deuterium, halogen, hydroxyl, and C₁₋₆ alkoxy;
each R¹⁰ is selected independently from the group consisting of C₁₋₆ alkyl and C₁₋₆ haloalkyl, optionally substituted with substituents independently selected from the group consisting of deuterium, hydroxyl, and C₁₋₆ alkoxy; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
each R^{a} and R^{b} is independently selected from H, C₁₋₆ alkyl, C(O)—O—C₁₋₆ alkyl, C(O)—O—C₂₋₆ alkenyl, —(CH₂)₀₋₂—C₃₋₇ cycloalkyl, and 3-7 membered heterocycloalkyl, wherein each alkyl, cycloalkyl or heterocycloalkyl is optionally substituted by 1-3 substituents selected from halogen and C₁₋₆ alkoxy; or
R^{a} and R^{b} can be taken together with the nitrogen to which they are attached to form a 4-7 membered ring;
m is 1 or 2;
n is 1, 2, or 3;
p is 0, 1, 2, 3, 4, 5, 6, 7, or 8;
wherein m+n is 2, 3, or 4; and
R¹ and R² are not both aryl.

2. The compound of claim 1 of formula (Ia) 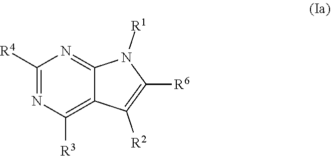
or a pharmaceutically acceptable salt thereof, wherein
R¹ is heteroaryl optionally substituted by 1-5 independently selected R⁷;
R² is C₁₋₆ alkyl optionally substituted by 1-5 independently selected R⁸;
R³ is 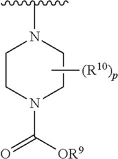
each of R⁴ and R⁶ is H;
each of R⁷ and R⁸ are independently selected at each occurrence from the group consisting of deuterium, hydroxy, halogen, cyano, C₁₋₆ alkyl, C₁₋₆ alkoxy, C₁₋₆ alkylthio, C₃₋₇ cycloalkyl, and NR^{a}R^{b}, wherein each C₁₋₆ alkyl and C₁₋₆ alkoxy are optionally substituted with substituents independently selected from the group consisting of halogen, hydroxyl, and C₁₋₆ alkoxy, and each C₃₋₇ cycloalkyl is optionally substituted with substituents independently selected from the group consisting of halogen, hydroxyl, C₁₋₆ alkoxy and C₁₋₆ alkyl;
R⁹ is C₁₋₆ alkyl, optionally substituted with substituents independently selected from the group consisting of deuterium, halogen, hydroxyl, and C₁₋₆ alkoxy;
each R¹⁰ is selected independently from the group consisting of C₁₋₆ alkyl and C₁₋₆ haloalkyl, optionally substituted with substituents independently selected from the group consisting of deuterium, hydroxyl, and C₁₋₆ alkoxy; or
two R¹⁰ on the same carbon can be taken together to form oxo; or
any two R¹⁰ can be taken together with the atoms to which they are attached to form an edge fused or spiro fused ring of 3-7 members, or a bridge of 1 to 3 carbons or a single bond, wherein the ring or bridge is optionally substituted with 1-3 substituents independently selected from the group consisting of halogen, hydroxyl, C₁₋₆ haloalkyl, and C₁₋₆ alkyl;
each R^{a} and R^{b} is independently selected from H, C₁₋₆ alkyl, C(O)—O—C₁₋₆ alkyl, C(O)—O—C₂₋₆ alkenyl, —(CH₂)₀₋₂—C₃₋₇ cycloalkyl, and 3-7 membered heterocycloalkyl, wherein each alkyl, cycloalkyl or heterocycloalkyl is optionally substituted by 1-3 substituents selected from halogen and C₁₋₆ alkoxy; or
R^{a} and R^{b} can be taken together with the nitrogen to which they are attached to form a 4-7 membered ring; and
p is 0, 1, 2, 3, 4, 5, 6, 7, or 8.

3. The compound of claim 1 of formula (Ia) 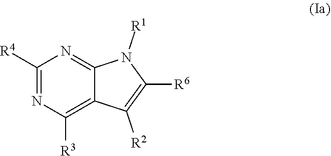
or a pharmaceutically acceptable salt thereof, wherein
R¹ is pyridyl optionally substituted by 1-5 independently selected R⁷;
R² is C₁₋₆ alkyl optionally substituted by 1-5 independently selected R⁸;
R³ is 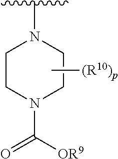
each of R⁴ and R⁶ is H;
each of R⁷ and R⁸ are independently selected at each occurrence from the group consisting of deuterium, hydroxy, halogen, cyano, C₁₋₆ alkyl, and C₁₋₆ alkoxy;
R⁹ is C₁₋₆ alkyl, optionally substituted with substituents independently selected from the group consisting of deuterium, halogen, hydroxyl, and C₁₋₆ alkoxy;
each R¹⁰ is selected independently from the group consisting of C₁₋₆ alkyl, and C₁₋₆ haloalkyl, each optionally substituted with 1-5 deuterium; and
p is 0, 1, 2, 3, 4, 5, 6, 7, or 8.

4. The compound of claim 1, or a pharmaceutically acceptable salt thereof, wherein R¹ is aryl optionally substituted by 1-5 independently selected R⁷.

5. The compound of claim 4, or a pharmaceutically acceptable salt thereof, wherein R¹ is phenyl optionally substituted with 1-3 independently selected R⁷.

6. The compound of claim 4, or a pharmaceutically acceptable salt thereof, wherein each R⁷ is independently selected from H, halogen, cyano, C₁₋₆ alkyl, C₁₋₆ alkoxy, C₁₋₆ haloalkyl, or C₁₋₆ haloalkoxy.

7. The compound of claim 1, or a pharmaceutically acceptable salt thereof, wherein R¹ is heteroaryl, cycloalkyl, or heterocycloalkyl, each R¹ optionally substituted by 1-5 independently selected R⁷.

8-12. (canceled)

13. The compound of claim 1, or a pharmaceutically acceptable salt thereof, wherein R¹ is pyridine, pyrimidine, pyrazine, pyridazine, thiazole, oxazole, pyrrole, imidazole, or pyrazole, pyridyl, tetrahydropyran, azetidine, pyrrolidine, morpholine, or piperidine, optionally substituted by 1-4 independently selected R⁷.

14-16. (canceled)

17. The compound of claim 1, or a pharmaceutically acceptable salt thereof, wherein R¹ is

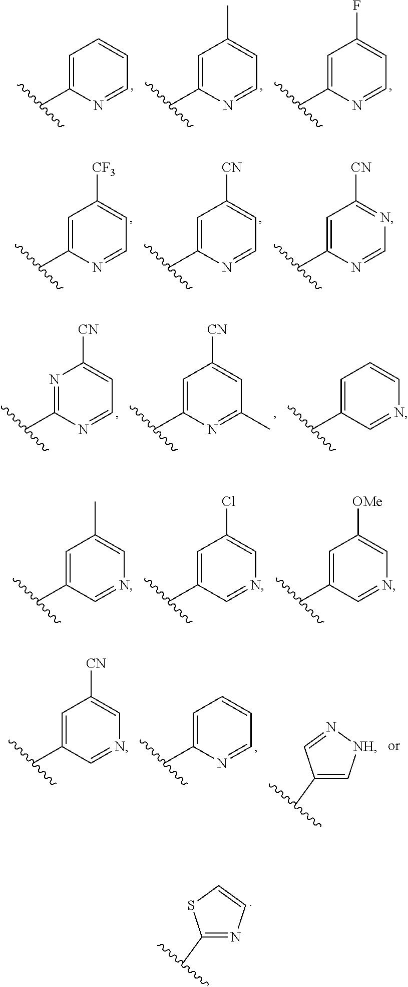

18-24. (canceled)

25. The compound of claim 1, or a pharmaceutically acceptable salt thereof, wherein R² is C₁₋₆ alkyl optionally substituted by 1-5 independently selected halogens.

26. The compound of claim 25, or a pharmaceutically acceptable salt thereof, wherein R² is Me, Et, CHF₂, or CF₃.

27. (canceled)

28. The compound of claim 1, or a pharmaceutically acceptable salt thereof, wherein m is 1 and n is 1.

29-33. (canceled)

34. The compound of claim 1, or a pharmaceutically acceptable salt thereof, wherein R³ is 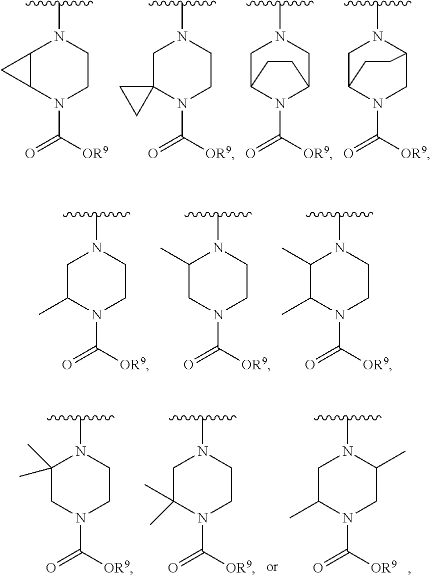
and R³ is optionally additionally substituted by 1-4 R¹⁰.

35-39. (canceled)

40. The compound of claim 1, or a pharmaceutically acceptable salt thereof, wherein R⁴ and R⁶ are H.

41-43. (canceled)

44. The compound of claim 40, or a pharmaceutically acceptable salt thereof, wherein R⁹ is ethyl, isopropyl, or t-butyl; each optionally substituted with 1-5 halogens or 1-9 deuteriums.

45-50. (canceled)

51. A compound of claim 1, or a pharmaceutically acceptable salt thereof, wherein the compound achieves at least 50% of the maximal current obtained with 30 μM ML-SA1 in a patch clamp assay for TRPML1, and has an EC₅₀ less than 1 μM or wherein the compound achieves a maximal current obtained with 30 μM ML-SA1 in a patch clamp assay for TRPML1 which is at least 10 fold the maximal current achieved for any other TRPML.

52. (canceled)

53. A pharmaceutical composition comprising a pharmaceutically acceptable excipient and a compound of claim 1 or a pharmaceutically acceptable salt thereof.

54. A method of modulating TRPML ion channels, the method comprising administering to a patient in need thereof a therapeutically effective amount of a compound of claim 1 or a pharmaceutically acceptable salt thereof.

55. A method of treating a disease or disorder which can be treated by modulation of TRPML ion channels, or by modulation of lysosomes, the method comprising administering to a patient in need thereof a therapeutically effective amount of a compound of claim 1 or a pharmaceutically acceptable salt thereof.

56. (canceled)

57. A method of treating a disorder selected from the group consisting of a ciliopathy, neurodegenerative disease, lysosomal storage disorder, lysosomal transport disorder, glycogen storage disorder, cholesteryl ester storage disease, a muscular disease, a disease related to aging, macular degeneration, and cancer, the method comprising administering to a patient in need thereof a therapeutically effective amount of a compound of claim 1 or a pharmaceutically acceptable salt thereof.

58-70. (canceled)
